(19) United States
(12) Patent Application Publication
(10) Pub. No.: US 2025/0263426 A1
(43) Pub. Date: Aug. 21, 2025

(54) PDE4B INHIBITOR AND USE THEREOF

(71) Applicant: Xizang Haisco Pharmaceutical Co., Ltd., Lhoka, Tibet (CN)

(72) Inventors: Yao LI, Lhoka, Tibet (CN); Lei CHEN, Lhoka, Tibet (CN); Zongjun SHI, Lhoka, Tibet (CN); Lei REN, Lhoka, Tibet (CN); Pengxin GENG, Lhoka, Tibet (CN); Jie WANG, Lhoka, Tibet (CN); Fengkai CHENG, Lhoka, Tibet (CN); Xiao LIN, Lhoka, Tibet (CN); Shuai HUANG, Lhoka, Tibet (CN); Xiaohai ZHANG, Lhoka, Tibet (CN); Shuang YANG, Lhoka, Tibet (CN); Pingming TANG, Lhoka, Tibet (CN); Chen ZHANG, Lhoka, Tibet (CN); Pangke YAN, Lhoka, Tibet (CN)

(21) Appl. No.: 19/102,113

(22) PCT Filed: Aug. 9, 2023
(86) PCT No.: PCT/CN2023/112061

§ 371 Date: Feb. 7, 2025

(30) Foreign Application Priority Data
Aug. 9, 2022 (CN) 202210950666.8
Sep. 2, 2022 (CN) 202211075118.1
Oct. 27, 2022 (CN) 202211323499.0
Nov. 24, 2022 (CN) 202211744316.2
Jan. 12, 2023 (CN) 202310041300.3
Feb. 24, 2023 (CN) 202310161801.5
Mar. 20, 2023 (CN) 202310269232.6
Apr. 7, 2023 (CN) 202310366104.3
Apr. 23, 2023 (CN) 202310441682.9
May 16, 2023 (CN) 202310550529.X
Jun. 7, 2023 (CN) 202310667178.0
Jul. 25, 2023 (CN) 202310911699.6

(51) Int. Cl. C07F 7/08; A61K 31/454; A61K 31/4545; A61K 31/506; A61K 31/519; A61K 31/5383; A61K 31/542; A61K 31/55; A61K 31/695; C07D 401/14; C07D 495/04; C07D 513/04; C07D 519/00

(52) U.S. Cl. CPC C07F 7/0816; A61K 31/454; A61K 31/4545; A61K 31/506; A61K 31/519; A61K 31/5383; A61K 31/542; A61K 31/55; A61K 31/695; C07D 401/14; C07D 495/04; C07D 513/04; C07D 519/00; C07F 7/0807

(57) ABSTRACT

A compound represented by formula I, stereoisomers or pharmaceutically acceptable salts thereof, or a pharmaceutical composition containing same, and the use thereof as a PDE4B inhibitor in the preparation of a drug for treatment of related diseases. Each group in formula (I) is as defined in the description.

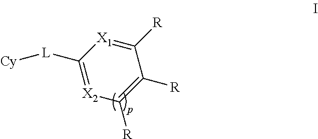

### CROSS-REFERENCE TO RELATED APPLICATION

This application is a 35 U.S.C. § 371 National Stage of International Patent Application No. PCT/CN2023/112061, filed Aug. 9, 2023, designating the United States, which claims priority to and the benefits of Chinese Patent Application No. 202210950666.8, filed Aug. 9, 2022, Chinese Patent Application No. 202211075118.1, filed Sep. 2, 2022, Chinese Patent Application No. 202211323499.0, filed Oct. 27, 2022, Chinese Patent Application No. 202211744316.2, filed Nov. 24, 2022, Chinese Patent Application No. 202310041300.3, filed Jan. 12, 2023, Chinese Patent Application No. 202310161801.5, filed Feb. 24, 2023, Chinese Patent Application No. 202310269232.6, filed Mar. 20, 2023, Chinese Patent Application No. 202310366104.3e, Chinese Patent Application No. 202310441682.9, filed Apr. 23, 2023, Chinese Patent Application No. 202310550529.X, filed May 16, 2023, Chinese Patent Application No. 202310667178.0, filed Jun. 7, 2023, Chinese Patent Application No. 202310911699.6, filed Jul. 25, 2023, the disclosures of which are incorporated herein in their entirety by reference, and priority is claimed to each of the foregoing.

### TECHNICAL FIELD

The present disclosure belongs to the field of medicine and in particular relates to a small molecule compound and a stereoisomer or pharmaceutically acceptable salt thereof, which have a selective inhibitory activity against PDE4B, and the use thereof in the preparation of a drug for treating a related disease.

### BACKGROUND

PDE4 inhibitors produce antidepressant effects in humans and animals by enhancing cAMP signaling in the brain. PDE4 inhibitors also play an important role in the treatment of other central nervous system diseases, including diseases such as Alzheimer's disease, Parkinson's disease, schizophrenia, stroke, and Huntington's chorea. In addition, the research and development of PDE4 inhibitors have also made great progress in the treatment of respiratory diseases such as asthma and chronic obstructive pulmonary disease. The principle of research and development of such drugs stems from the role of PDE4 in inhibiting the function of a range of inflammatory cells and resident cells, which is believed to be associated with the pathogenesis of such diseases. A large number of clinical studies have shown that cyclic adenosine monophosphate (cAMP) can block the proliferation and chemotaxis of inflammatory cells and inhibit the release of inflammatory and cytotoxic mediators in lungs. In addition, PDE4 is especially abundant in immune cells, inflammatory cells, and smooth muscle cells.

PDE4 inhibitors play an anti-inflammatory role mainly by inhibiting the hydrolysis function of PDE4 so as to increase the cAMP level in vivo, thereby inhibiting the release of inflammatory factors and at the same time promoting the production of anti-inflammatory mediators. Roflumilast is used clinically for the treatment of COPD with a significant anti-inflammatory effect and can inhibit the release of inflammatory mediators such as TNF-α, interleukins, and chemokines from monocytes, macrophages, T cells, etc. However, such inhibitors generally have serious side effects such as nausea and vomiting, which limits the clinical application of PDE4 inhibitors. A large number of studies have shown that phosphodiesterase 4 subtype B (PDE4B), which is associated with inflammatory responses in the human body, participates in the release of various inflammatory mediators in vivo, while subtype D is closely related to the production of side effects such as nausea and vomiting, which provides a new idea for finding PDE4 inhibitors with low side effects, i.e., designing PDE4B inhibitors that may have reduced side effects, thereby promoting further clinical application.

Phosphodiesterase 4 is highly selective for cAMP and has four subtypes, namely PDE4A, 4B, 4C, and 4D, with at least 25 splice variants. The protein sequences of the catalytic domains of the four subtypes of PDE4 are highly homologous; therefore, inhibitors that act on the catalytic domain do not exhibit subtype selectivity. Most of the classical PDE4 inhibitors have been reported to act on the catalytic domain. A new mode of action of PDE4 inhibitors has been reported in recent years, in which the inhibitor acts on both the catalytic domain and a regulatory sequence, so that the regulatory sequence can stabilize the closed conformation of the protein, thereby preventing cAMP from entering, thus exerting an inhibitory effect. However, it has been found from research that there are amino acid differences between PDE4B and 4D in this regulatory sequence, so designing inhibitors based on such differences can produce subtype selectivity. Therefore, based on the difference of the two amino acids PDE4B Leu674/PDE4D Gln594 in CR3 (Conserved Region 3) of the downstream regulatory sequence, it is expected to achieve the selectivity to subtype B and reduce the side effects of the inhibitors while maintaining the activity.

Selective PDE4B inhibitors with good activity, high safety and minor side effects are found, which have a good clinical development prospect and can be used for treating COPD or cancers or other proliferative diseases or conditions.

### SUMMARY

The present disclosure provides a small molecule compound with PDE4B inhibitory activity, or a stereoisomer or pharmaceutically acceptable salt thereof, wherein the compound is as represented by formula (I), and has a high activity, relatively low toxic and side effects, excellent pharmacokinetic characteristics, and bioavailability;

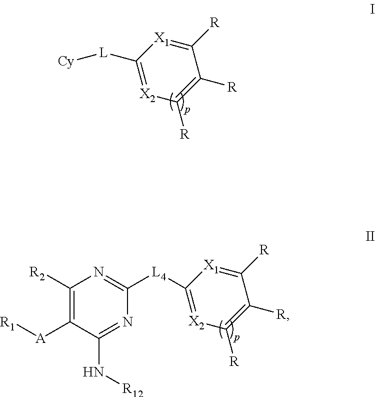

wherein when Cy is selected from Cy1, Cy2, Cy3, Cy8, and Cy10, L is -(L₁)n-(L₂)m-;

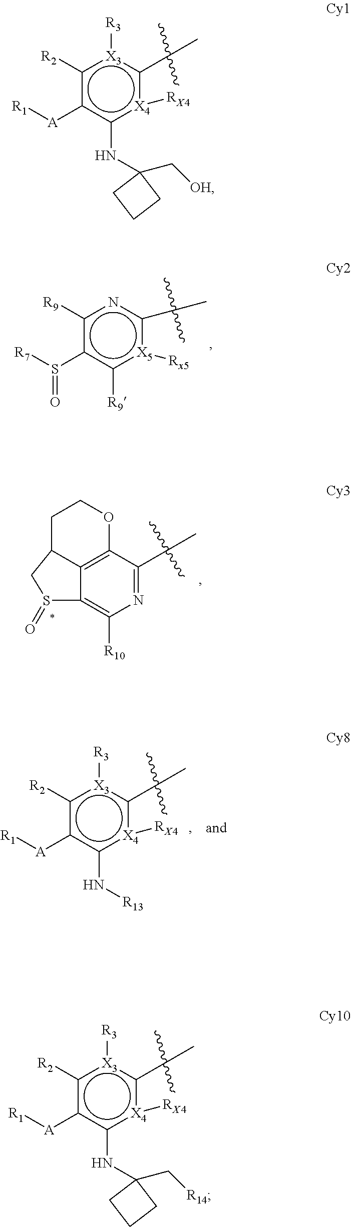

when Cy is selected from Cy4, Cy5, Cy6, and Cy11, L is -L₃-;

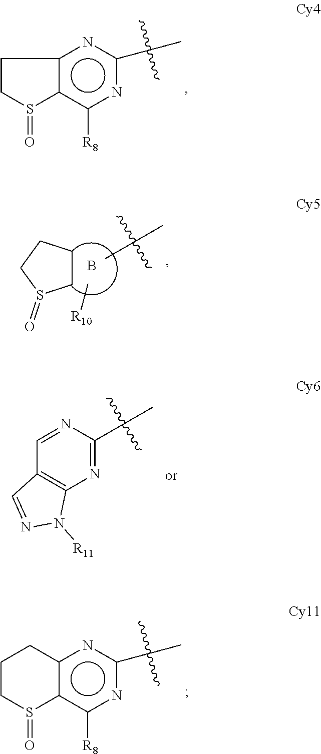

when Cy is selected from Cy7 and Cy9, L is -L₄-;

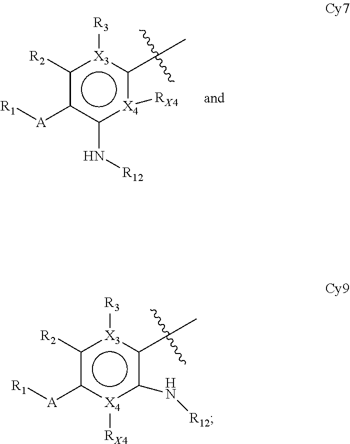

L₁ is —NR₄—, —CR₅═N—, —CR₅=CR₅—, or —CR₅R₅—; in all the solutions of the present disclosure, unless otherwise specially specified, the site of attachment of L₁ is arbitrary, for example, as for L₁(-CR₂=N—) that is connected to L₂, the N atom and C atom thereof can both be used as the site of attachment to L₂;
L₂ is

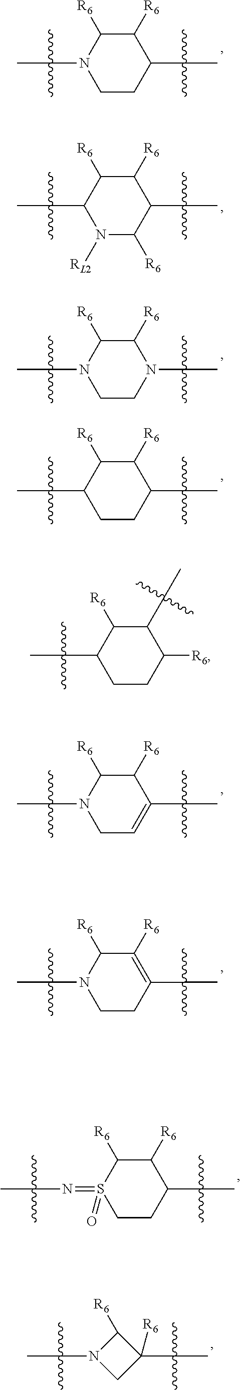

C₁₋₄ alkylene, C₂₋₄ alkenylene, C₂₋₄ alkynylene, CO, O, NR_{L2}, a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 10-membered fused heterocycloalkyl containing 1-3 heteroatoms selected from N, S, and O, a 6- to 10-membered bridged heterocycloalkyl containing 1-3 heteroatoms selected from N, S, and O, or a 7- to 12-membered spiro heterocycloalkyl containing 1-3 heteroatoms selected from N, S, and O, wherein the alkylene, alkenylene, alkynylene, monocyclic heterocycloalkyl, fused heterocycloalkyl, bridged heterocycloalkyl, and spiro heterocycloalkyl are optionally substituted with 1-3 groups selected from halogen, ═O, CN, C₁₋₄ alkyl, C₁₋₄ alkoxy, OH, and NH₂; in some embodiments, each L₂ is

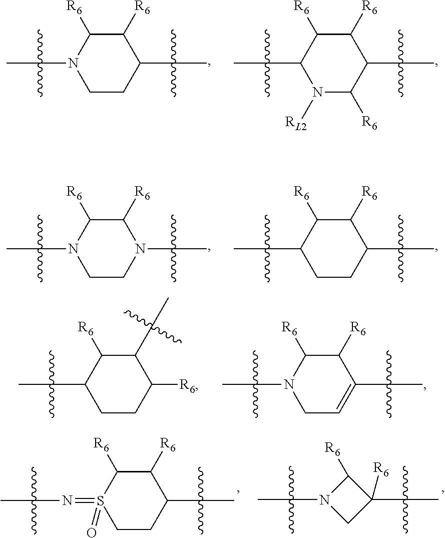

C₁₋₄ alkylene, C₂₋₄ alkenylene, C₂₋₄ alkynylene, CO, O, NR_{L2}, a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 10-membered fused heterocycloalkyl containing 1-3 heteroatoms selected from N, S, and O, a 6- to 10-membered bridged heterocycloalkyl containing 1-3 heteroatoms selected from N, S, and O, or a 7- to 12-membered spiro heterocycloalkyl containing 1-3 heteroatoms selected from N, S, and O, wherein the alkylene, alkenylene, alkynylene, monocyclic heterocycloalkyl, fused heterocycloalkyl, bridged heterocycloalkyl, and spiro heterocycloalkyl are optionally substituted with 1-3 groups selected from halogen, ═O, CN, C₁₋₄ alkyl, C₁₋₄ alkoxy, OH, and NH₂; in some embodiments, each L₂ is independently selected from

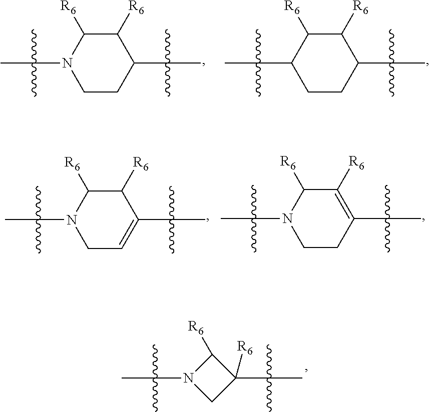

CO, O, NR_{L2}, C₁₋₄ alkylene, C₂₋₄ alkenylene, C₂₋₄ alkynylene,

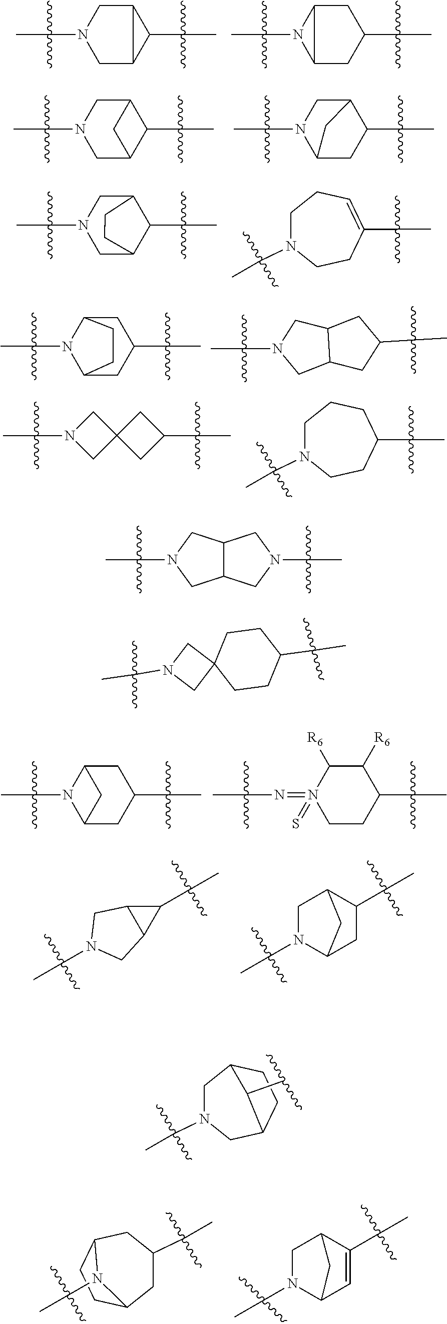

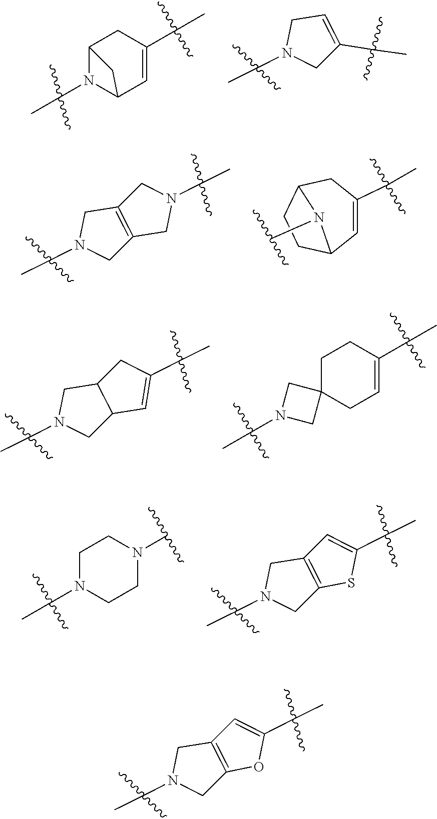

some embodiments, each L₂ is independently selected from

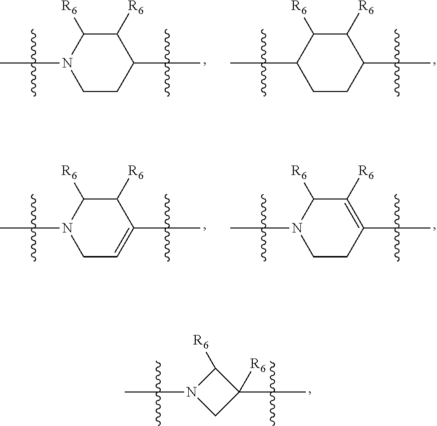

CO, O, NR_{L2}, C₁₋₄ alkylene C₂₋₄ alkenylene, C₂₋₄ alkynylene,

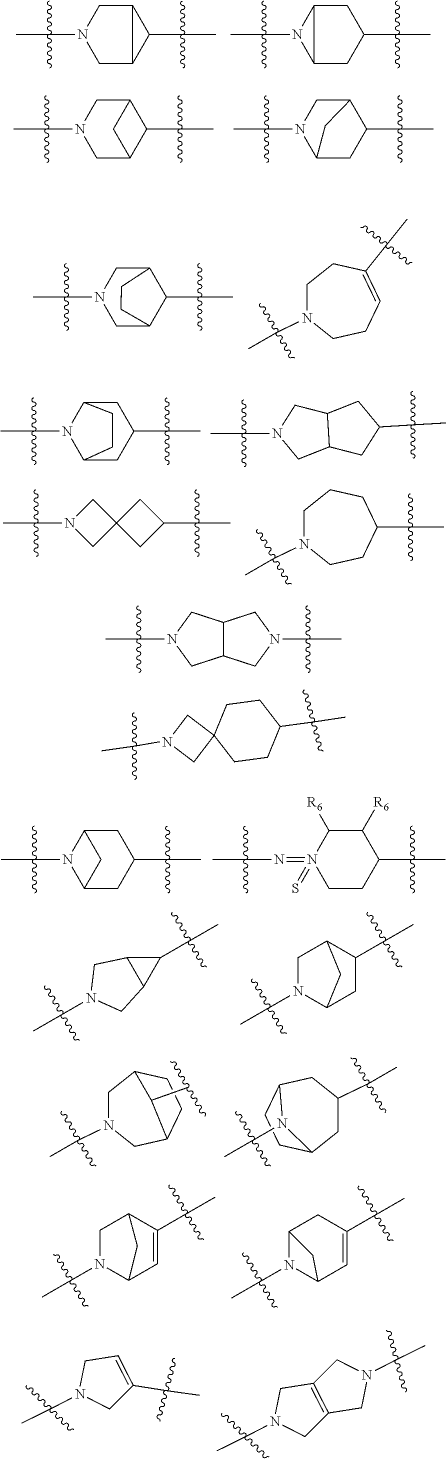

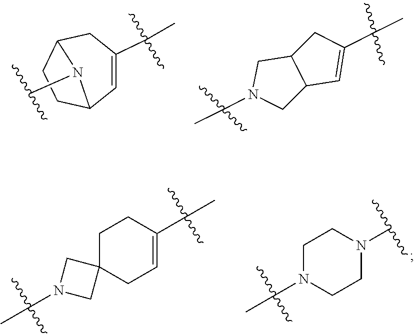 [SMILES: *C1=CC2CCC(C1)N2* ; *C1=CC2CN(*)CC2C1 ; *C1=CCC2(CC1)CN(*)C2 ; *N1CCN(*)CC1]

in some embodiments each L₂ is independently selected from

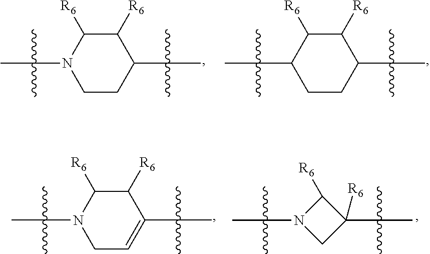

CO, O, NR_{L2}, C₁₋₄ alkylene, C₂₋₄ alkenylene, C₂₋₄ alkynylene,

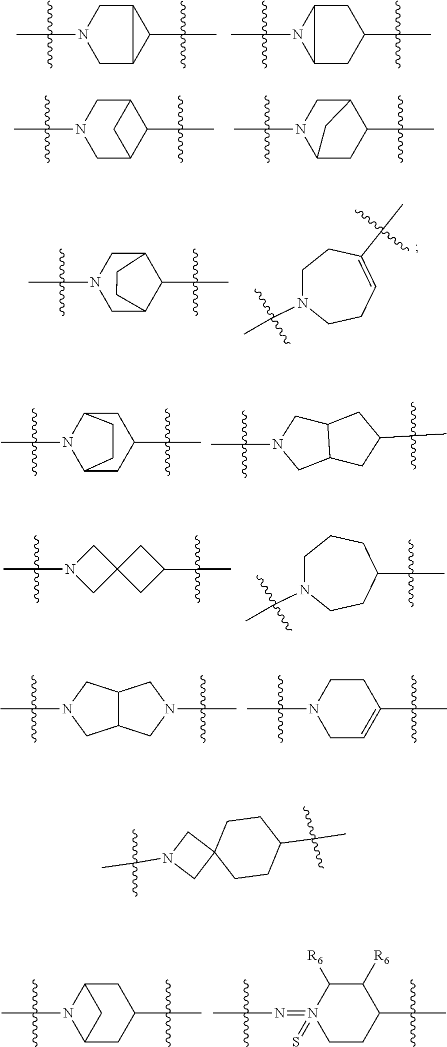

in all the solutions of the present disclosure, unless otherwise specially specified, the sites of attachment of L₂ to the left and right groups are arbitrary, for example, in L₂

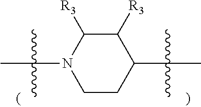

as one end, the N atom and C atom thereof can both be used as the site of attachment to Cy;
R_{L2} is H, C₁₋₄ alkyl, or C₃₋₆ cycloalkyl; in some embodiments, R_{L2} is H, C₁₋₄ alkyl, or C₃₋₆ cycloalkyl; in some embodiments, R_{L2} is H, methyl, ethyl, propyl, or isopropyl;
L₃ is heterocycloalkylene or heterocycloalkylene-(CH₂)ᵣ—, wherein the heterocycloalkylene is a 4- to 10-membered heterocycle containing 1-3 heteroatoms selected from N, S, and O and is optionally substituted with 1-3 R_{L3}; in some embodiments, L₃ is selected from

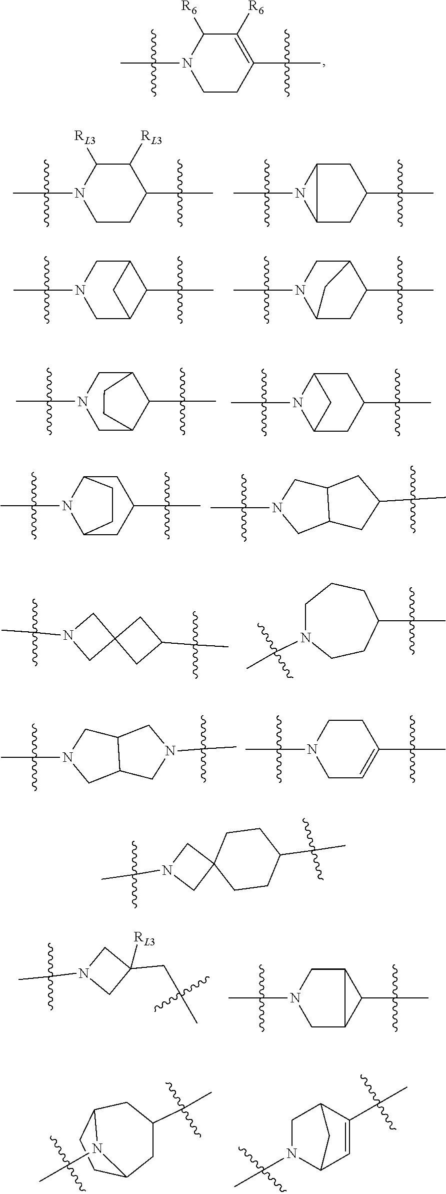

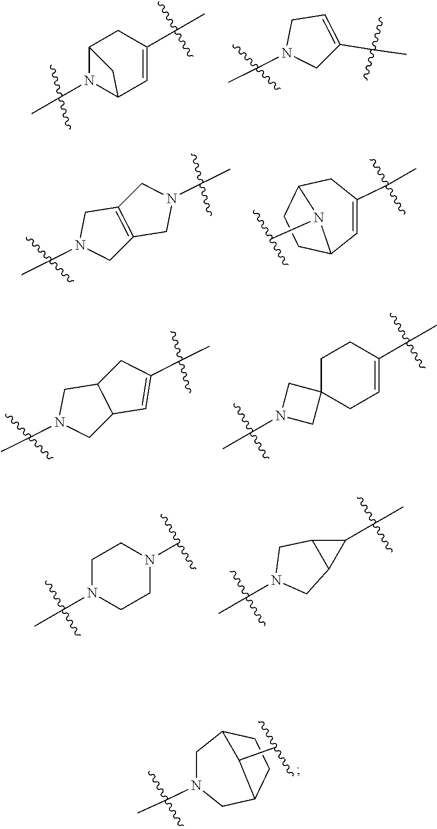

in some embodiments, L₃ is selected from

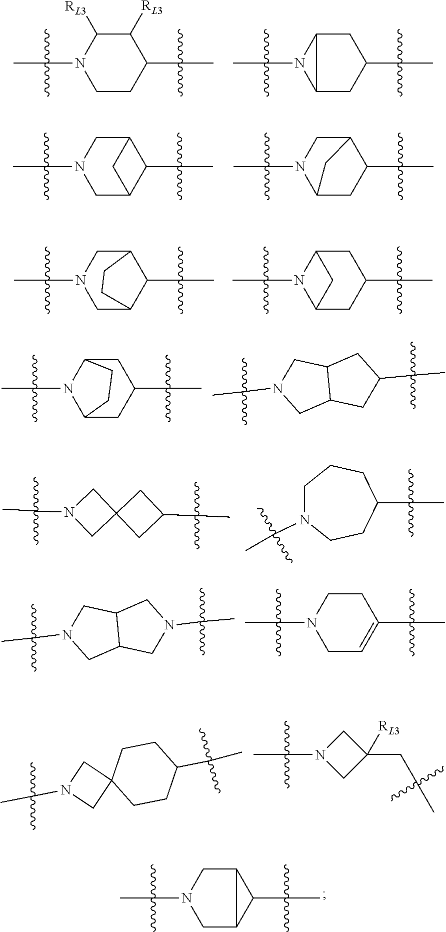

each R_{L3} is independently selected from halogen, ═O, CN, C₁₋₄ alkyl, or C₁₋₄ alkoxy, or R_{L3} and R_{X1}, together with the atom to which they are attached, form C₃₋₆ cycloalkyl or a 5- to 8-membered heterocycloalkyl containing 1-3 heteroatoms selected from N, S, and O; in some embodiments, each R_{L3} is independently selected from halogen, ═O, CN, C₁₋₄ alkyl, or C₁₋₄ alkoxy, or R_{L3} and R_{X1}, together with the atom to which they are attached, form a C₃₋₆ cycloalkyl or a 5- to 6-membered heterocycloalkyl containing 1-3 heteroatoms selected from N, S, and O;
L₄ is

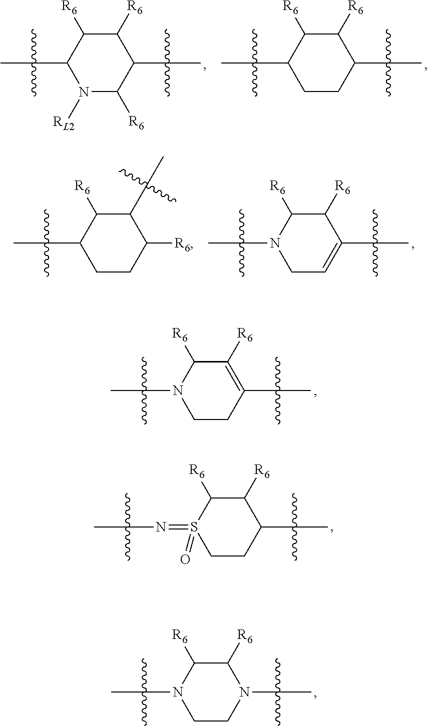

a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 6-membered heterocycloalkyl fused 5- to 6-membered heterocycloalkyl containing 1-3 heteroatoms selected from N, S, and O, a 5- to 6-membered heterocycloalkyl fused 5- to 6-membered cycloalkyl containing 1-3 heteroatoms selected from N, S, and O, or a 7- to 12-membered spiro heterocycloalkyl containing 1-3 heteroatoms selected from N, S, and O, wherein the monocyclic heterocycloalkyl, 5- to 6-membered heterocycloalkyl fused 5- to 6-membered heterocycloalkyl, 5- to 6-membered heterocycloalkyl fused 5- to 6-membered cycloalkyl, and spiro heterocycloalkyl are optionally substituted with 1-3 groups selected from halogen, ═O, CN, C₁₋₄ alkyl, C₁₋₄ alkoxy, OH, and NH₂;
in some embodiments, L₄ is

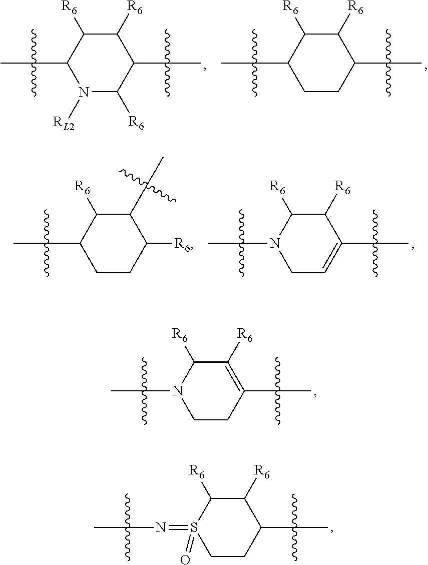

a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 6-membered heterocycloalkyl fused 5- to 6-membered heterocycloalkyl containing 1-3 heteroatoms selected from N, S, and O, a 5- to 6-membered heterocycloalkyl fused 5- to 6-membered cycloalkyl containing 1-3 heteroatoms selected from N, S, and O, or a 7- to 12-membered spiro heterocycloalkyl containing 1-3 heteroatoms selected from N, S, and O, wherein the monocyclic heterocycloalkyl, 5- to 6-membered heterocycloalkyl fused 5- to 6-membered heterocycloalkyl, 5- to 6-membered heterocycloalkyl fused 5- to 6-membered cycloalkyl, and spiro heterocycloalkyl are optionally substituted with 1-3 groups selected from halogen, ═O, CN, C₁₋₄ alkyl, C₁₋₄ alkoxy, OH, and NH₂; in some embodiments, L₄ is

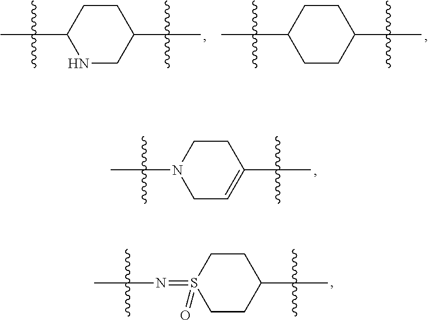 [SMILES: *C1CCC(*)NC1 ; *C1CCC(*)CC1 ; *C1=CCN(*)CC1 ; *N=S1(=O)CCC(*)CC1]

a 5- to 6-membered heterocycloalkyl fused 5- to 6-membered heterocycloalkyl containing 1-3 heteroatoms selected from N, S, and O, or a 5- to 6-membered heterocycloalkyl fused 5- to 6-membered cycloalkyl containing 1-3 heteroatoms selected from N, S, and O, wherein the 5- to 6-membered heterocycloalkyl fused 5- to 6-membered heterocycloalkyl and 5- to 6-membered heterocycloalkyl fused 5- to 6-membered cycloalkyl are optionally substituted with 1-3 groups selected from halogen, ═O, CN, C₁₋₄ alkyl, C₁₋₄ alkoxy, OH, and NH₂; in some embodiments, L₄ is selected from

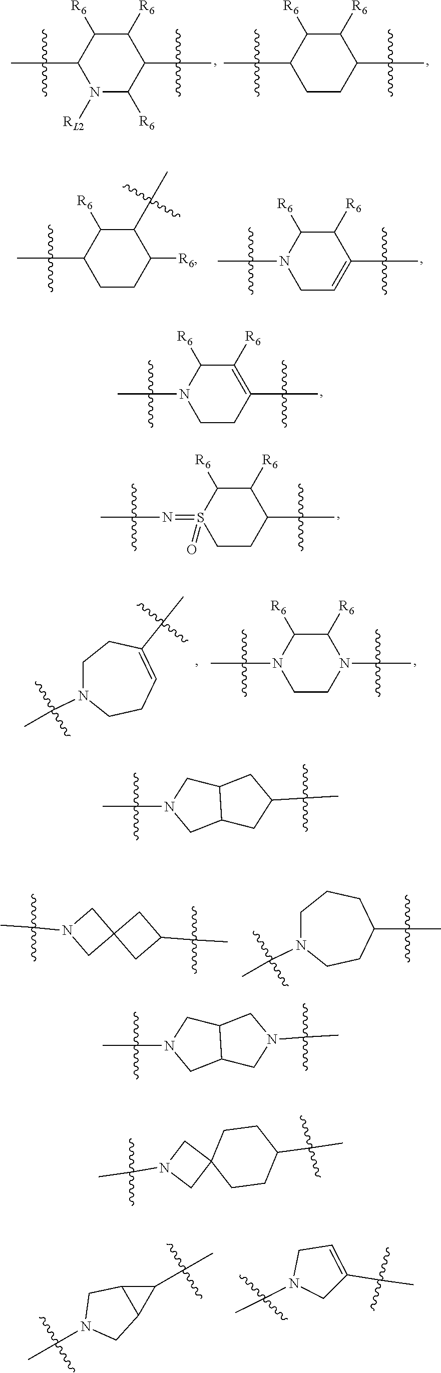

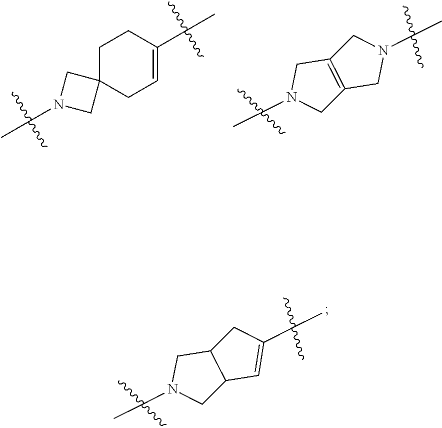 [SMILES: *C1=CCC2(CC1)CN(*)C2 ; *N1CC2=C(C1)CN(*)C2 ; *C1=CC2CN(*)CC2C1]

in some embodiments, L₄ is selected from

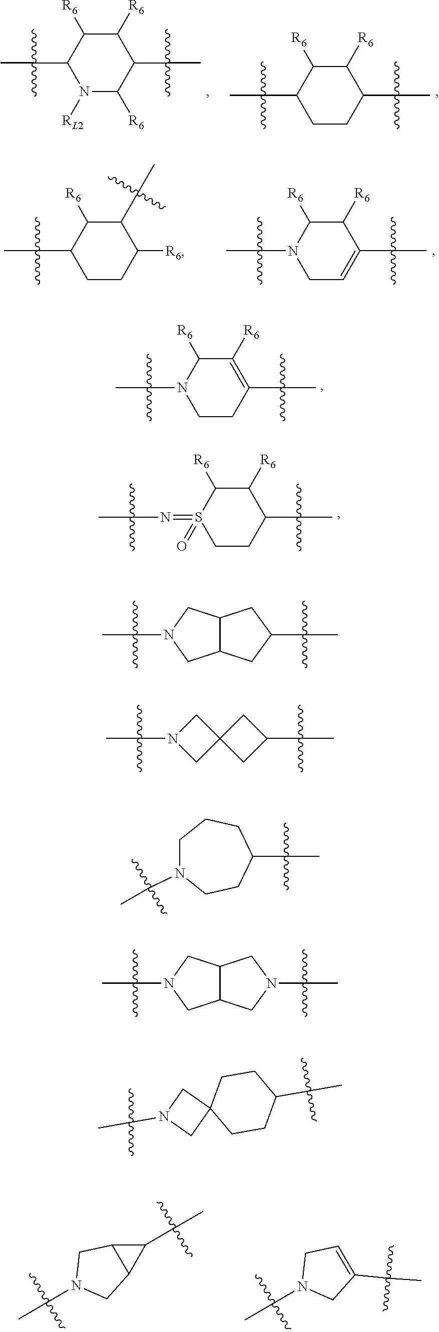

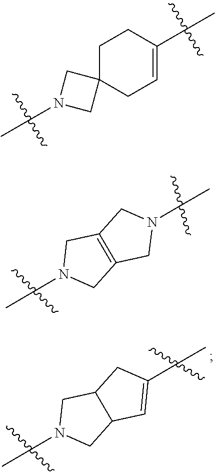 [SMILES: *C1=CCC2(CC1)CN(*)C2 ; *N1CC2=C(C1)CN(*)C2 ; *C1=CC2CN(*)CC2C1]

in some embodiments, L₄ is

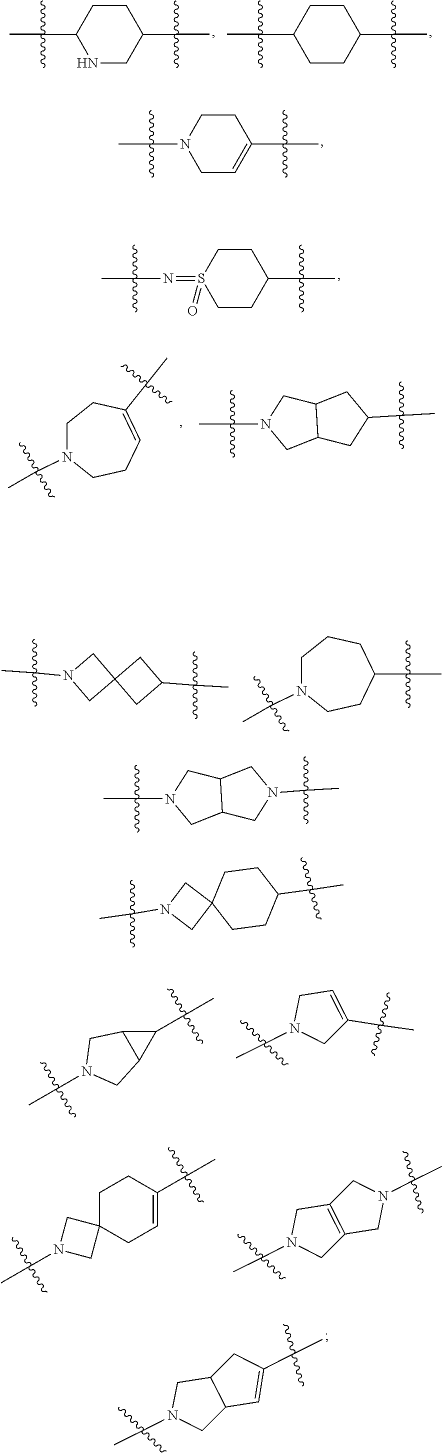

in some embodiments L₄ is

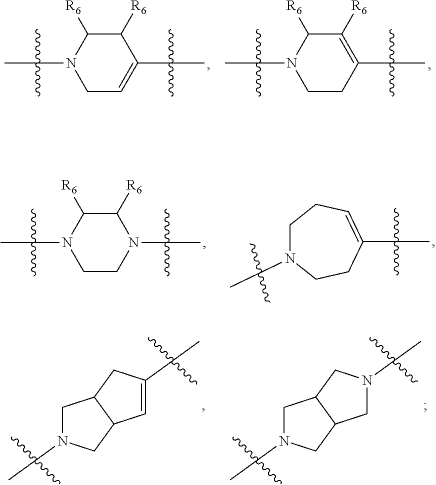

in some embodiments, L₄ is

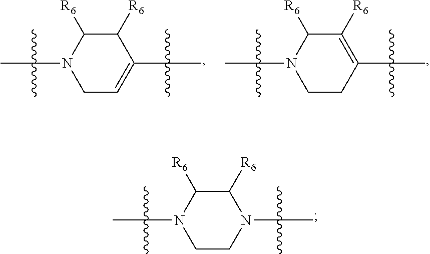

in some embodiments, L₄ is

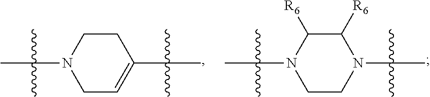

A is —S(O)—, —C(O)—, —B(OH)—, —S—, —S(═N)—CN, or —C(═N)—CN; in some embodiments, A is —S(O)—, —C(O)—, —B(OH)—, —S—, or —S(═N)—CN; in some embodiments, A is —S(O)—; X₁ and X₂ are independently CR_{X1}, O, S, or N; in some embodiments, X₁ and X₂ are independently CR_{X1}, S, or N; in some embodiments, X₁ and X₂ are independently S, or N; in some embodiments, X₁ and X₂ are independently CR_{X1} or N; in some embodiments, X₁ and X₂ are CR_{X1}; in some embodiments, X₁ and X₂ are independently N; X₃, X₄, and X₅ are independently C or N; in some embodiments, X₃ and X₄ are independently N;
ring B is heteroaryl or heterocycloalkyl fused heteroaryl, wherein the heteroaryl is a 5-membered heteroaryl containing 1-3 heteroatoms selected from N, S, and O, and the heterocycloalkyl is a 5- to 6-membered heterocycloalkyl containing 1-3 heteroatoms selected from N, S, and O; in some embodiments, Cy5 is selected from

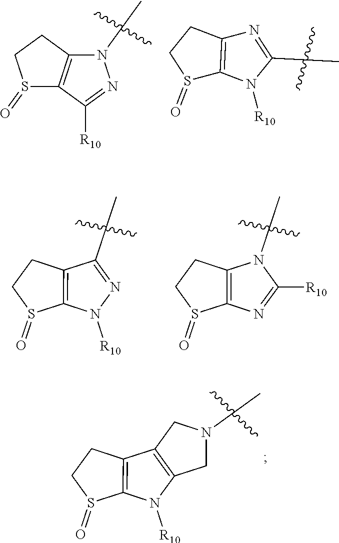

each R_{X1} and R are independently H, halogen, C₁₋₄ alkyl, C₁₋₄ alkoxy, —N(CH₃)₂, —NH(CH₃), C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, O, and Si, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂; in some embodiments, each R_{X1} and R are independently H, halogen, C₁₋₄ alkyl, C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, O, and Si, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, C₃₋₆ cycloalkyl, C₂₋₆ alkynyl, and NH₂; in some embodiments, each R_{X1} and R are independently H, halogen, C₁₋₄ alkyl, C₂₋₄ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₆ cycloalkyl or a 5- to 6-membered heterocycloalkyl containing 1-3 heteroatoms selected from N, S, O, and Si, wherein the cycloalkyl and heterocycloalkyl are optionally substituted with 1-3 groups selected from halogen, ═O, CN, C₁₋₄ alkyl, C₁₋₄ alkoxy, OH, C₃₋₆ cycloalkyl, C₂₋₆ alkynyl, and NH₂; in some embodiments, each R_{X1} and R are independently H, halogen, C₁₋₄ alkyl, C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, O, and Si, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂; in some embodiments, each R_{X1} and R are independently H, halogen, C₁₋₄ alkyl, C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, or C₁₋₄ haloalkyl, or R_{X1} and R are independently 3- to 5-membered cycloalkyl, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, and O, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂; in some embodiments, each R_{X1} and R are independently H, halogen, C₁₋₄ alkyl, C₂₋₄ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, or C₁₋₄ haloalkyl, or two R on adjacent ring atoms, R and R_{X1}, or R_{X1} and R₆, together with the atom to which they are attached, form C₃₋₆ cycloalkyl or a 5- to 6-membered heterocycloalkyl containing 1-3 heteroatoms selected from N, S, and O, wherein the cycloalkyl and heterocycloalkyl are optionally substituted with 1-3 groups selected from halogen, ═O, CN, C₁₋₄ alkyl, C₁₋₄ alkoxy, OH, and NH₂; in some embodiments, each R_{X1} and R are independently H, halogen, C₁₋₄ alkyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, or C₁₋₄ haloalkyl, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, and O, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂; in some embodiments, each R_{X1} and R are independently H, halogen, C₁₋₄ alkyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, or C₁₋₄ haloalkyl, or two R on adjacent ring atoms, R and R_{X1}, or R_{X1} and R₆, together with the atom to which they are attached, form C₃₋₆ cycloalkyl, wherein the cycloalkyl is optionally substituted with 1-3 groups selected from halogen, ═O, CN, C₁₋₄ alkyl, C₁₋₄ alkoxy, OH, and NH₂; in some embodiments, each R_{X1} and R are independently H, halogen, C₁₋₄ alkyl, C₂₋₄ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₆ cycloalkyl or a 5- to 6-membered heterocycloalkyl containing 1-3 heteroatoms selected from N, S, O, and Si, wherein the cycloalkyl and heterocycloalkyl are optionally substituted with 1-3 groups selected from halogen, ═O, CN, C₁₋₄ alkyl, C₁₋₄ alkoxy, OH, and NH₂;
R₁ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₁ and R₂, together with the atom to which they are attached, form a 5- to 10-membered heterocycloalkyl or a 5- to 6-membered heteroaryl containing 1-3 heteroatoms selected from N, S, B, and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂; in some embodiments, R₁ is C₁₋₄ alkyl, or C₁₋₄ haloalkyl, or R₁ and R₂, together with the atom to which they are attached, form a 5- to 7-membered monocyclic heterocycloalkyl, a 6- to 8-membered fused heterocycloalkyl, a 7- to 9-membered spiro heterocycloalkyl, or a 5- to 6-membered heteroaryl containing 1-3 heteroatoms selected from N, S, B, and O, wherein the monocyclic heterocycloalkyl, fused heterocycloalkyl, spiro heterocycloalkyl, and heteroaryl are optionally substituted with 1-3 groups selected from halogen, ═O, CN, C₁₋₄ alkyl, C₁₋₄ alkoxy, OH, and NH₂; in some embodiments, R₁ is C₁₋₄ alkyl, or C₁₋₄ haloalkyl; or R₁ and R₂, together with the atom to which they are attached, form a ring selected from:

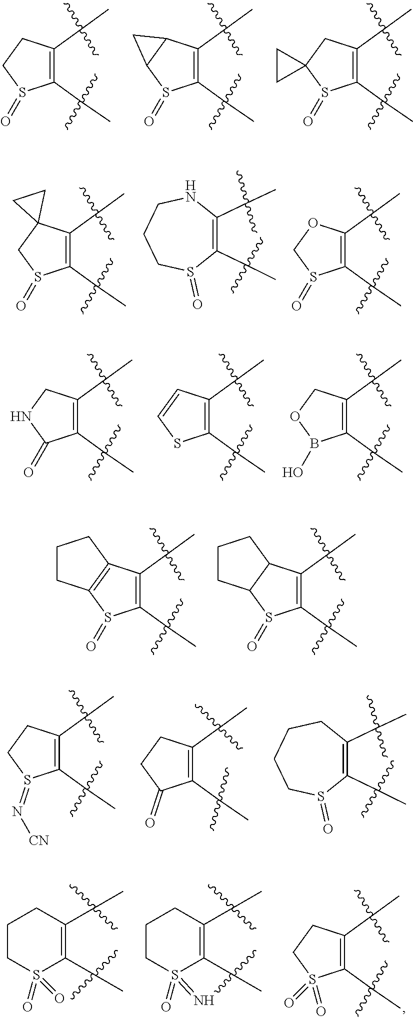

optionally substituted with 1-3 groups selected from halogen, ═O, CN, C₁₋₄ alkyl, C₁₋₄ alkoxy, OH, and NH₂;
or R₁ and R₂, together with the atom to which they are attached, form a ring selected from:

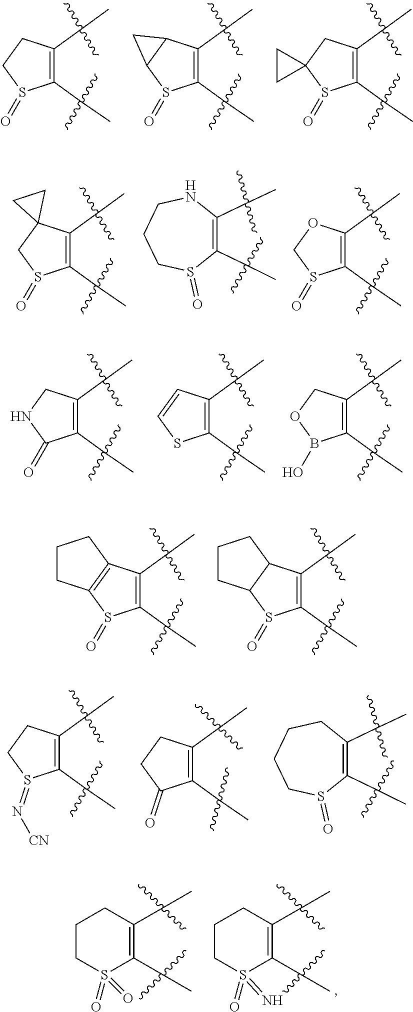

optionally substituted with 1-3 groups selected from halogen, ═O, CN, C₁₋₄ alkyl, C₁₋₄ alkoxy, OH, and NH₂;
or R₁ and R₂, together with the atom to which they are attached, form a ring selected from:

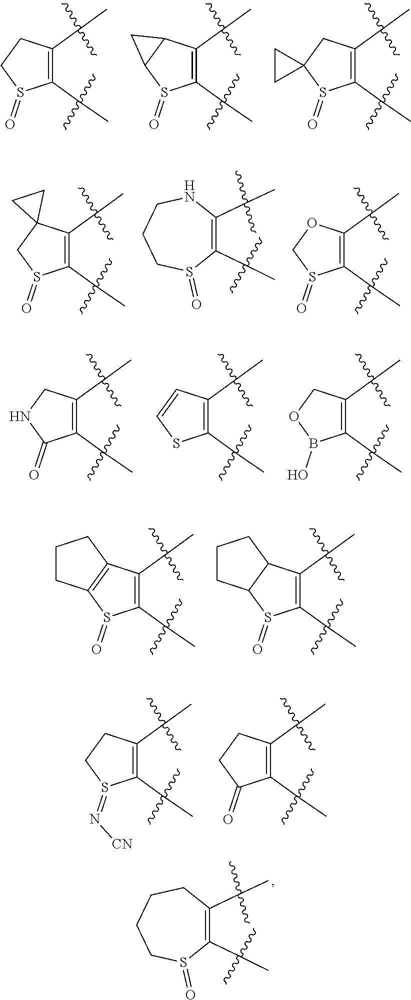

optionally substituted with 1-3 groups selected from halogen, ═O, CN, C₁₋₄ alkyl, C₁₋₄ alkoxy, OH, and NH₂; or R₁ and R₂, together with the atom to which they are attached, form a ring selected from:

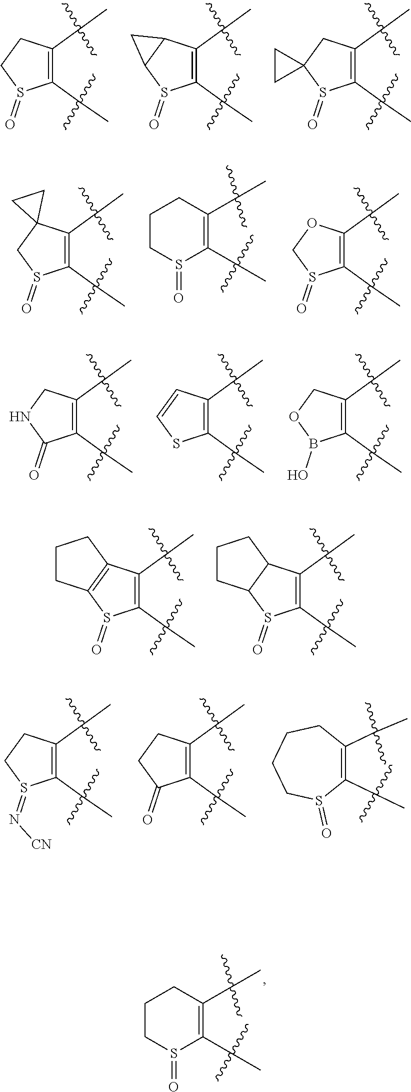

optionally substituted with 1-3 groups selected from halogen, ═O, CN, C₁₋₄ alkyl, C₁₋₄ alkoxy, OH, and NH₂; in some embodiments, R₁ and R₂, together with the atom to which they are attached, form a ring selected from:

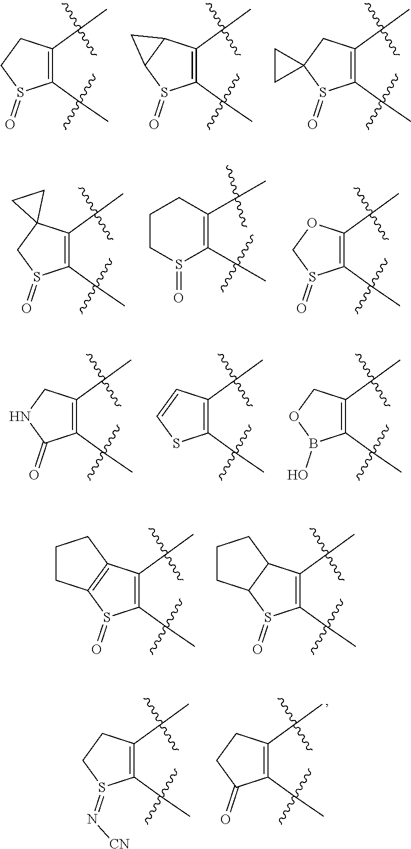

optionally substituted with 1-3 groups selected from halogen, ═O, CN, C₁₋₄ alkyl, C₁₋₄ alkoxy, OH, and NH₂;
R₂ is H, C₁₋₄ alkyl, or C₁₋₄ haloalkyl; in some embodiments, R₂ is H, C₁₋₄ alkyl, or C₁₋₄ haloalkyl; in some embodiments, R₃ and R₂, together with the atom to which they are attached, form a ring selected from:

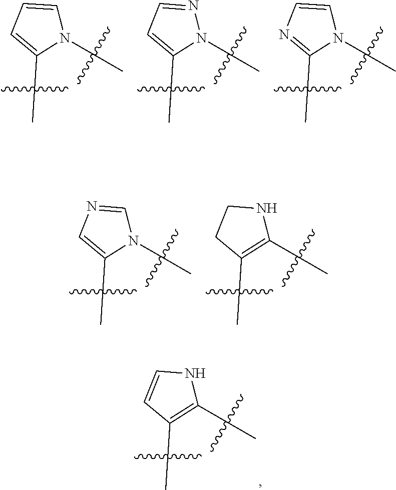 [SMILES: *c1cccn1* ; *c1ccnn1* ; *c1nccn1* ; *c1cncn1* ; *C1=C(*)NCC1 ; *c1cc[nH]c1*]

optionally substituted with 1-3 groups selected from halogen, ═O, CN, C₁₋₄ alkyl, C₁₋₄ alkoxy, OH, and NH₂;
R₃ is H, halogen, C₁₋₄ alkyl, CN, OH, or absent, or R₃ and R₂, R₃ and R₆, R₃ and R₄, or R₃ and R₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂; in some embodiments, R₃ is H, halogen, C₁₋₄ alkyl, CN, OH, or absent; or R₃ and R₂, together with the atom to which they are attached, form a 5- to 6-membered heterocycloalkyl or a 5- to 6-membered heteroaryl containing 1-3 heteroatoms selected from N, S, and O, wherein the heterocycloalkyl and heteroaryl are optionally substituted with 1-3 groups selected from halogen, ═O, CN, C₁₋₄ alkyl, C₁₋₄ alkoxy, OH, and NH₂; or R₃ and R₄ or R₃ and R₅, together with the atom to which they are attached, form a 5- to 6-membered heterocycloalkyl or a 5- to 6-membered heteroaryl containing 1-3 heteroatoms selected from N, S, and O, or phenyl, wherein the heterocycloalkyl, phenyl, and heteroaryl are optionally substituted with 1-3 groups selected from halogen, ═O, CN, C₁₋₄ alkyl, C₁₋₄ alkoxy, OH, and NH₂;
or R₃ and R₆, together with the atom to which they are attached, form C₅₋₇ cycloalkyl, a 5- to 6-membered heterocycloalkyl, or a 5- to 6-membered heteroaryl containing 1-3 heteroatoms selected from N, S, and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
in some embodiments, R₃ is H, halogen, C₁₋₄ alkyl, CN, OH, or absent; or R₃ and R₂, together with the atom to which they are attached, form a ring selected from:

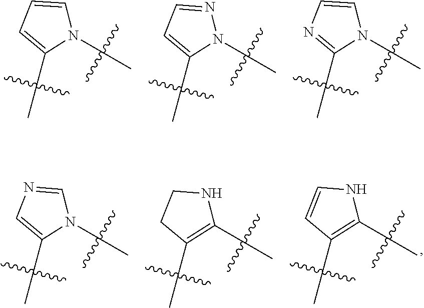 [SMILES: *c1cccn1* ; *c1ccnn1* ; *c1nccn1* ; *c1cncn1* ; *C1=C(*)NCC1 ; *c1cc[nH]c1*]

optionally substituted with 1-3 groups selected from halogen, ═O, CN, C₁₋₄ alkyl, C₁₋₄ alkoxy, OH, and NH₂; or R₃ and R₄ or R₃ and R₅, together with the atom to which they are attached, form a 5- to 6-membered heterocycloalkyl or a 5- to 6-membered heteroaryl containing 1-3 heteroatoms selected from N, S, and O, or phenyl, wherein the heterocycloalkyl, phenyl, and heteroaryl are optionally substituted with 1-3 groups selected from halogen, ═O, CN, C₁₋₄ alkyl, C₁₋₄ alkoxy, OH, and NH₂;
or R₃ and R₆, together with the atom to which they are attached, form C₅₋₆ cycloalkyl, or a 5- to 6-membered heterocycloalkyl containing 1-3 heteroatoms selected from N, S, and O, optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent, or Rₓ₄ and R₄, Rₓ₄ and R₅, or Rₓ₄ and R₆, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂; in some embodiments, Rₓ₄ is H, halogen, C₁₋₄ alkyl, CN, OH, or absent, or Rₓ₄ and R₄ or Rₓ₄ and R₅, together with the atom to which they are attached, form a 5- to 6-membered heteroaryl containing 1-3 heteroatoms selected from N, S, and O, wherein the heteroaryl is optionally substituted with 1-3 groups selected from halogen, ═O, CN, C₁₋₄ alkyl, C₁₋₄ alkoxy, OH, and NH₂; in some embodiments, Rₓ₄ is H, halogen, C₁₋₄ alkyl, CN, OH, or absent; or Rₓ₄ and R₄ or Rₓ₄ and R₅, together with the atom to which they are attached, form imidazolyl, pyrazolyl, pyrrolyl, thienyl, or thiazolyl, optionally substituted with 1-3 groups selected from halogen, ═O, CN, C₁₋₄ alkyl, C₁₋₄ alkoxy, OH, and NH₂;
R₄ is H, C₁₋₄ alkyl, C₁₋₄ haloalkyl, or C₃₋₆ cycloalkyl; in some embodiments, R₄ is H, C₁₋₄ alkyl, or C₁₋₄ haloalkyl; in some embodiments, R₄ is H, methyl, ethyl, propyl, or isopropyl, or halogenated methyl, ethyl, propyl, or isopropyl, with the halogen including F, Cl, Br, and I;
R₅ is H, C₁₋₄ alkyl, C₁₋₄ haloalkyl, C₁₋₄ alkoxy, —NHC₁₋₄ alkyl, or C₃₋₆ cycloalkyl; in some embodiments, R₅ is H, C₁₋₄ alkyl, C₁₋₄ haloalkyl, C₁₋₄ alkoxy, or —NHC₁₋₄ alkyl;
R₆ is H, halogen, ═O, CN, C₁₋₄ alkyl, or C₁₋₄ alkoxy; in some embodiments, R₆ is H, halogen, ═O, CN, methyl, ethyl, propyl, isopropyl, methoxy, ethoxy, propoxy, or isopropoxy;
Rₓ₅ is C₁₋₄ alkyl, CN, OH, or absent; in some embodiments, Rₓ₅ is methyl, ethyl, propyl, isopropyl, CN, OH, or absent;
R₇ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₇ and R₉, together with the atom to which they are attached, form a 5- to 6-membered heterocycloalkyl containing 1-3 heteroatoms selected from N, S, and O, wherein the alkyl, haloalkyl, cycloalkyl, and heterocycloalkyl are optionally substituted with 1-3 groups selected from halogen, CN, C₁₋₄ alkyl, C₁₋₄ alkoxy, OH, and NH₂; in some embodiments, R₇ is C₁₋₄ alkyl, or C₁₋₄ haloalkyl, or R₇ and R₉, together with the atom to which they are attached, form a 5- to 6-membered heterocycloalkyl containing 1-3 heteroatoms selected from N, S, and O, wherein the heterocycloalkyl is optionally substituted with 1-3 groups selected from halogen, CN, C₁₋₄ alkyl, C₁₋₄ alkoxy, OH, and NH₂; in some embodiments, R₇ is C₁₋₄ alkyl, or C₁₋₄ haloalkyl, or R₇ and R₉, together with the atom to which they are attached, form a ring selected from:

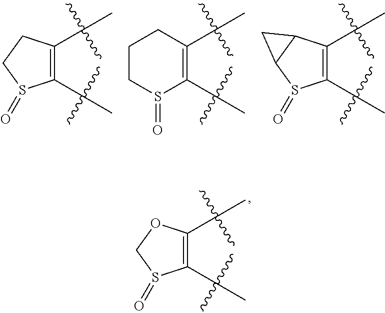

wherein the ring is optionally substituted with 1-3 groups selected from halogen, CN, C₁₋₄ alkyl, C₁₋₄ alkoxy, OH, and NH₂; in the present disclosure, at least one group of R_{9′} and R₇ or R_{9′} and Rₓ₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the cycloalkyl, phenyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′}; in some embodiments, at least one group of R_{9′} and R₇ or R_{9′} and Rₓ₅, together with the atom to which they are attached, form a 5- to 7-membered monocyclic heterocycloalkyl containing 1-3 heteroatoms selected from N, S, and O, a 7- to 10-membered spiro heterocycloalkyl, or a 5- to 6-membered heteroaryl, wherein the monocyclic heterocycloalkyl, spiro heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′}; and in some embodiments, R_{9′} and R₇, together with the atom to which they are attached, form a ring selected from:

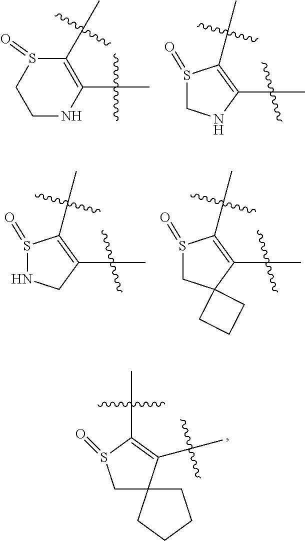 [SMILES: *C1=C(*)S(=O)CCN1 ; *C1=C(*)S(=O)CN1 ; *C1=C(*)S(=O)NC1 ; *C1=C(*)C2(CCC2)CS1=O ; *C1=C(*)C2(CCCC2)CS1=O]

optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
or R_{9′} and Rₓ₅, together with the atom to which they are attached, form

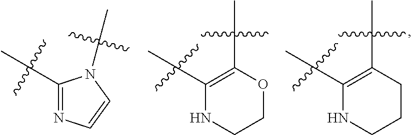 [SMILES: *c1nccn1* ; *C1=C(*)OCCN1 ; *C1=C(*)NCCC1]

optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′}; provided that at least one group of R_{9′} and R₇ or R_{9′} and Rₓ₅ form a ring;
R₈ is R_{8′}, —OR_{8′}, or —(CH₂)ᵣR_{8′};
R_{8′} is C₃₋₆ cycloalkyl, a 4- to 8-membered heterocycloalkyl containing 1-3 heteroatoms selected from N, S, O, Si, and P, a 5- to 6-membered heteroaryl containing 1-3 heteroatoms selected from N, S, and O, or —N═S(═O)(C₁₋₄ alkyl)₂, or —N(C₃₋₆ cycloalkyl)C(O)NH(C₁₋₄ alkyl), wherein the cycloalkyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, and CN; in some embodiments, R_{8′} is C₃₋₆ cycloalkyl, a 4- to 8-membered heterocycloalkyl containing 1-3 heteroatoms selected from N, S, and O, or a 5- to 6-membered heteroaryl containing 1-3 heteroatoms selected from N, S, and O, wherein the cycloalkyl, heterocycloalkyl, and heteroaryl are optionally substituted with 1-3 groups selected from C₁₋₄ alkyl, hydroxy C₁. 4 alkyl, mercapto C₁₋₄ alkyl, halogen, halo C₁₋₄ alkyl, ═O, OH, NH₂, and CN; in some embodiments, R_{8′} is C₃₋₆ cycloalkyl, wherein the cycloalkyl is optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, and CN;
R₉ is H, C₁₋₄ alkyl, or C₁₋₄ haloalkyl; in some embodiments, R₉ is H or C₁₋₄ alkyl; in some embodiments, R₉ is H, methyl, ethyl, propyl, or isopropyl;
each R₁₀ is independently R_{8′}, —OR_{8′}, —NHR_{8′}, or —(CH₂)ᵣR_{8′};
R₁₁ is C₃₋₆ cycloalkyl optionally substituted with 1-3 groups selected from halogen, CN, ═O, OH, C₁₋₄ alkyl, hydroxy C₁₋₄ alkyl, and C₁₋₄ haloalkyl; in some embodiments, the cycloalkyl is optionally substituted with 1-3 F, Cl, Br, I, CN, ═O, OH, methyl, ethyl, propyl, isopropyl, hydroxymethyl, hydroxyethyl, hydroxypropyl, or hydroxyisopropyl, or halogenated methyl, ethyl, propyl, or isopropyl, etc., with the halogen including F, Cl, Br, and I;
R₁₂ is selected from a 4- to 10-membered heterocycloalkylene, a 3- to 10-membered cycloalkylene, a 6- to 10-membered arylene, a 5- to 10-membered heteroarylene, hydroxy-C₁₋₆ alkyl, C₁₋₄ alkyl-CN, —CRaRb=N—O—C₁₋₄ alkyl, —C(NRaRb)=N—CN, —C(C₁₋₄ alkyl)=N—CN, —C(NRaRb)=CRa—NO₂, —C(CH₃)₂—CH₂—O—C(O)NH₂, —C(CH₃)₂—CH₂—NH—C(O)O(C₁₋₄ alkyl), —C(CH₃)₂—CH₂—O—C(O)NHCH₃, —CH(CH₃)—CH₂—O—C(O)NH₂, —CH(CH₃)—CH₂—NH—C(O)O(C₁₋₄ alkyl), —CH(CH₃)—CH₂—O—C(O)NHCH₃, —C(O)—Ra, or —C(O)-(4- to 6-membered heterocycloalkyl), wherein the heterocycloalkylene, cycloalkylene, arylene, and heteroarylene are optionally further substituted with 1-3 groups selected from halogen, CN, ═O, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NRa—C(O)NH₂, —(CH₂)ₜ—NRa—C(═NH)NH₂, —(CH₂)ₜ—NRa—C(O)—O—(C₁₋₄ alkyl), —(CH₂)ₜ—NRa—C(C₁₋₄ alkyl)=N—CN, —(CH₂)ₜ—NRa—C(NRaRb)=N—CN, —(CH₂)ₜ—NRa—C(NRaRb)=CRa—NO₂, —(CH₂)ₜ—C(NRaRb)=N—CN, —(CH₂)ₜ—C(C₁₋₄ alkyl)=N—CN, —(CH₂)ₜ—C(C₁₋₄ alkyl)=N—O—(C₁₋₄ alkyl), ═N—O—C₁₋₄ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃;
in some embodiments, R₁₂ is selected from a 4- to 10-membered heterocycloalkylene, a 3- to 10-membered cycloalkylene, a 6- to 10-membered arylene, a 5- to 10-membered heteroarylene, hydroxy-C₁₋₆ alkyl, —CRaRb=N—O—C₁₋₄ alkyl, —C(NRaRb)=N—CN, —C(C₁₋₄ alkyl)=N—CN, —C(NRaRb)=CRa—NO₂, —C(CH₃)₂—CH₂—O—C(O)NH₂, —C(CH₃)₂—CH₂—NH—C(O)O(C₁₋₄ alkyl), —C(CH₃)₂—CH₂—O—C(O)NHCH₃, —CH(CH₃)—CH₂—O—C(O)NH₂, —CH(CH₃)—CH₂—NH—C(O)O(C₁₋₄ alkyl), —CH(CH₃)—CH₂—O—C(O)NHCH₃, —C(O)—Ra, or —C(O)-(4- to 6-membered heterocycloalkyl), wherein the heterocycloalkylene, cycloalkylene, arylene, and heteroarylene are optionally further substituted with 1-3 groups selected from halogen, CN, ═O, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NRa—C(O)NH₂, —(CH₂)ₜ—NRa—C(═NH)NH₂, —(CH₂)ₜ—NRa—C(O)—O—(C₁₋₄ alkyl), —(CH₂)ₜ—NRa—C(C₁₋₄ alkyl)=N—CN, —(CH₂)ₜ—NRa—C(NRaRb)=N—CN, —(CH₂)ₜ—NRa—C(NRaRb)=CRa—NO₂, —(CH₂)ₜ—C(NRaRb)=N—CN, —(CH₂)ₜ—C(C₁₋₄ alkyl)=N—CN, —(CH₂)ₜ—C(C₁₋₄ alkyl)=N—O—(C₁₋₄ alkyl), ═N—O—C₁₋₄ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃;
in some embodiments, R₁₂ is selected from a 4- to 10-membered heterocycloalkylene, a 3- to 10-membered cycloalkylene, a 6- to 10-membered arylene, a 5- to 10-membered heteroarylene, hydroxy-C₁₋₆ alkyl, —CRaRb=N—O—C₁₋₄ alkyl, —C(NRaRb)=N—CN, —C(C₁₋₄ alkyl)=N—CN, —C(NRaRb)=CRa—NO₂, —C(CH₃)₂—CH₂—O—C(O)NH₂, —CH(CH₃)—CH₂—O—C(O)NH₂, —C(O)—Ra, —CH(CH₃)—CH₂—O—C(O)NHCH₃, or —C(CH₃)₂—CH₂—O—C(O)NHCH₃, wherein the heterocycloalkylene, cycloalkylene, arylene, and heteroarylene are optionally further substituted with 1-3 groups selected from halogen, CN, ═O, OH, —SF₅, C₁₋₄ alkyl, hydroxy C₁₋₄ alkyl, C₁₋₄ haloalkyl, —(CH₂)ₜ—O—C(O)NH₂, —(CH₂)ₜ—NRa—C(O)NH₂, —(CH₂)ₜ—NRa—C(═NH)NH₂, —(CH₂)ₜ—NRa—C(O)—O—(C₁₋₄ alkyl), —(CH₂)ₜ—NRa—C(C₁₋₄ alkyl)=N—CN, —(CH₂)ₜ—NRa—C(NRaRb)=N—CN, —(CH₂)ₜ—NRa—C(NRaRb)=CRa—NO₂, —(CH₂)ₜ—C(NRaRb)=N—CN, —(CH₂)ₜ—C(C₁₋₄ alkyl)=N—CN, —(CH₂)ₜ—C(C₁₋₄ alkyl)=N—O—(C₁₋₄ alkyl), ═N—O—C₁₋₄ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃; in some embodiments, R₁₂ is selected from a 4- to 10-membered heterocycloalkylene, a 3- to 10-membered cycloalkylene, a 6- to 10-membered arylene, a 5- to 10-membered heteroarylene, hydroxy C₁₋₆ alkyl, —CRaRb=N—O—C₁₋₄ alkyl, —C(NRaRb)=N—CN, —C(C₁₋₄ alkyl)=N—CN, —C(NRaRb)=CRa—NO₂, or —C(CH₃)ₜ—CH₂—O—C(O)NHCH₃, wherein the heterocycloalkylene, cycloalkylene, arylene, and heteroarylene are optionally further substituted with 1-3 groups selected from halogen, CN, ═O, OH, —SF₅, C₁₋₄ alkyl, hydroxy C₁₋₄ alkyl, C₁₋₄ haloalkyl, —(CH₂)ₜ—O—C(O)NH₂, —(CH₂)ₜ—NRa—C(O)NH₂, —(CH₂)ₜ—NRa—C(═NH)NH₂, —(CH₂)ₜ—NRa—C(O)—O—(C₁₋₄ alkyl), —(CH₂)ₜ—NRa—C(C₁₋₄ alkyl)=N—CN, —(CH₂)ₜ—NRa—C(NRaRb)=N—CN, —(CH₂)ₜ—NRa—C(NRaRb)=CRa—NO₂, —(CH₂)ₜ—C(NRaRb)=N—CN, —(CH₂)ₜ—C(C₁₋₄ alkyl)=N—CN, —(CH₂)ₜ—C(C₁₋₄ alkyl)=N—O—(C₁₋₄ alkyl), ═N—O—C₁₋₄ alkyl, and a 5- to 6-membered heteroaryl; in some embodiments, R₁₂ is selected from a 4- to 10-membered heterocycloalkylene, a 3- to 10-membered cycloalkylene, a 6- to 10-membered arylene, or a 5- to 10-membered heteroarylene, wherein the heterocycloalkylene, cycloalkylene, arylene, and heteroarylene are optionally further substituted with 1-3 groups selected from halogen, CN, ═O, OH, C₁₋₄ alkyl, hydroxy C₁₋₄ alkyl, and C₁₋₄ haloalkyl; in some embodiments, R₁₂ is selected from a 4- to 10-membered heterocycloalkylene and a 3- to 10-membered cycloalkylene, wherein the heterocycloalkylene and cycloalkylene are optionally further substituted with 1-3 groups selected from halogen, CN, ═O, OH, C₁₋₄ alkyl, hydroxy C₁₋₄ alkyl, and C₁₋₄ haloalkyl; in some embodiments, R₁₂ is selected from a 4- to 6-membered monocyclic heterocycloalkylene, a 6- to 10-membered fused heterocarbocyclylene, a 7- to 8-membered bridged heterocarbocyclylene, a 7- to 10-membered spiro heterocarbocyclylene, a 3- to 6-membered monocyclic cycloalkylene, a 6- to 10-membered fused carbocyclylene, a 7- to 8-membered bridged carbocyclylene, and a 7- to 10-membered spiro carbocyclic ring, wherein the monocyclic heterocycloalkylene, fused heterocarbocyclylene, bridged heterocarbocyclylene, spiro heterocarbocyclylene, monocyclic cycloalkylene, fused carbocyclylene, bridged carbocyclylene, and spiro carbocyclic ring are optionally further substituted with 1-3 groups selected from halogen, CN, ═O, OH, C₁₋₄ alkyl, hydroxy C₁₋₄ alkyl, and C₁₋₄ haloalkyl; in some embodiments, R₁₂ is selected from cyclobutyl, cyclopentyl, cyclohexyl, phenyl, cyclopropyl, adamantyl, tetrahydropyranyl, piperidinyl, oxolanyl, oxanyl,

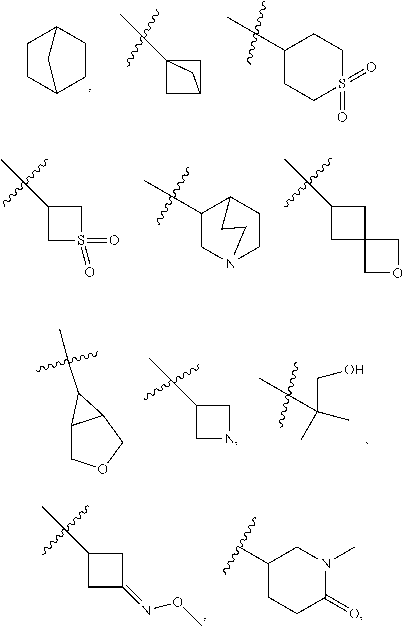

C₁₋₄ alkyl-CN, —C(NHC₁₋₂ alkyl)=N—CN, —C(C₁₋₂ alkyl)=N—CN, —C(NHC₁₋₂ alkyl)=CH—NO₂, —CH(CH₃)—CH₂—NH—C(O)O(CH₃), —C(CH₃)₂—CH₂—NH—C(O)O(CH₃), —CH(CH₃)—CH₂—O—C(O)NH₂, —C(CH₃)₂—CH₂—O—C(O)NH₂, —C(O)—Ra, —CH(CH₃)—CH₂—O—C(O)NHCH₃, or —C(CH₃)₂—CH₂—O—C(O)NHCH₃; wherein the ring is optionally substituted with 1-3 groups selected from halogen, CN, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NH—C(O)NH₂, —(CH₂)ₜ—NH—C(═NH)NH₂, —(CH₂)ₜ—NH—C(O)—O—(C₁₋₂ alkyl), —(CH₂)ₜ—NH—C(C₁₋₂ alkyl)=N—CN, —(CH₂)ₜ—NH—C(NHC₁₋₂ alkyl)=N—CN, —(CH₂)ₜ—NH—C(NHC₁₋₂ alkyl)=CH—NO₂, —(CH₂)ₜ—C(NHC₁₋₂ alkyl)=N—CN, —(CH₂)ₜ—C(C₁₋₂ alkyl)=N—CN, —(CH₂)ₜ—C(C₁₋₂ alkyl)=N—O—(C₁₋₂ alkyl), ═N—O—C₁₋₂ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃; in some embodiments, R₁₂ is selected from cyclobutyl, cyclopentyl, cyclohexyl, phenyl, cyclopropyl, adamantyl, tetrahydropyranyl, piperidinyl, oxolanyl, oxanyl,

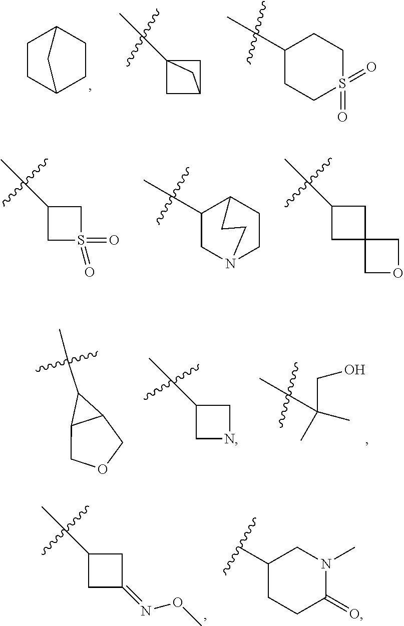

—C(NHC₁₋₂ alkyl)=N—CN, —C(C₁₋₂ alkyl)=N—CN, —C(NHC₁₋₂ alkyl)=CH—NO₂, —C(CH₃)₂—CH₂—NH—C(O)O(CH₃), —C(CH₃)₂—CH₂—O—C(O)NH₂, —CH(CH₃)—CH₂—NH—C(O)O(CH₃), —CH(CH₃)₂—CH₂—O—C(O)NH₂, —C(O)—Ra, —C(CH₃)₂—CH₂—O—C(O)NHCH₃, or —CH(CH₃)—CH₂—O—C(O)NHCH₃; wherein the ring is optionally substituted with 1-3 groups selected from halogen, CN, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NH—C(O)NH₂, —(CH₂)ₜ—NH—C(═NH)NH₂, —(CH₂)ₜ—NH—C(O)—O—(C₁₋₂ alkyl), —(CH₂)ₜ—NH—C(C₁₋₂ alkyl)=N—CN, —(CH₂)ₜ—NH—C(NHC₁₋₂ alkyl)=N—CN, —(CH₂)ₜ—NH—C(NHC₁₋₂ alkyl)=CH—NO₂, —(CH₂)ₜ—C(NHC₁₋₂ alkyl)=N—CN, —(CH₂)ₜ—C(C₁₋₂ alkyl)=N—CN, —(CH₂)ₜ—C(C₁₋₂ alkyl)=N—O—(C₁₋₂ alkyl), ═N—O—C₁₋₂ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃; in some embodiments, R₁₂ is selected from cyclobutyl, cyclopentyl, cyclohexyl, phenyl, cyclopropyl, adamantyl, tetrahydropyranyl, piperidinyl, oxolanyl, oxanyl,

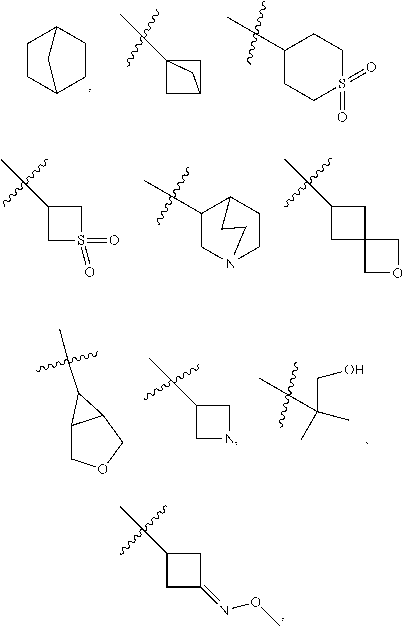

—C(NHC₁₋₂ alkyl)=N—CN, —C(C₁₋₂ alkyl)=N—CN, —C(NHC₁₋₂ alkyl)=CH—NO₂, —C(CH₃)₂—CH₂—NH—C(O)O(CH₃), —C(CH₃)₂—CH₂—O—C(O)NH₂, —CH(CH₃)—CH₂—NH—C(O)O(CH₃), —CH(CH₃)—CH₂—O—C(O)NH₂, —C(O)—Ra, —C(CH₃)₂—CH₂—O—C(O)NHCH₃, or —CH(CH₃)—CH₂—O—C(O)NHCH₃; wherein the ring is optionally substituted with 1-3 groups selected from halogen, CN, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NH—C(O)NH₂, —(CH₂)ₜ—NH—C(═NH)NH₂, —(CH₂)ₜ—NH—C(O)—O—(C₁₋₂ alkyl), —(CH₂)ₜ—NH—C(C₁₋₂ alkyl)=N—CN, —(CH₂)ₜ—NH—C(NHC₁₋₂ alkyl)=N—CN, —(CH₂)ₜ—NH—C(NHC₁₋₂ alkyl)=CH—NO₂, —(CH₂)ₜ—C(NHC₁₋₂ alkyl)=N—CN, —(CH₂)ₜ—C(C₁₋₂ alkyl)=N—CN, —(CH₂)ₜ—C(C₁₋₂ alkyl)=N—O—(C₁₋₂ alkyl), ═N—O—C₁₋₂ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃; in some embodiments, R₁₂ is selected from cyclobutyl, cyclopentyl, cyclohexyl, phenyl, cyclopropyl, adamantyl, tetrahydropyranyl, piperidinyl, oxolanyl, oxanyl,

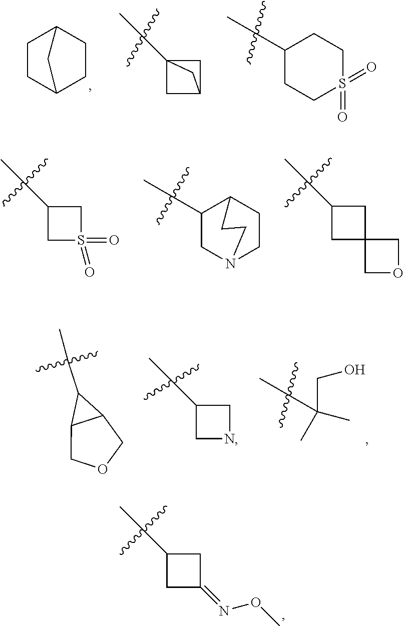

—C(NHC₁₋₂ alkyl)=N—CN, —C(C₁₋₂ alkyl)=N—CN, or —C(NHC₁₋₂ alkyl)=CH—NO₂; the ring is optionally substituted with 1-3 groups selected from halogen, CN, OH, —SF₅, C₁₋₄ alkyl, hydroxy C₁₋₄ alkyl, C₁₋₄ haloalkyl, —(CH₂)ₜ—O—C(O)NH₂, —(CH₂)ₜ—NH—C(O)NH₂, —(CH₂)ₜ—NH—C(═NH)NH₂, —(CH₂)ₜ—NH—C(O)—O—(C₁₋₂ alkyl), —(CH₂)ₜ—NH—C(C₁₋₂ alkyl)=N—CN, —(CH₂)ₜ—NH—C(NHC₁₋₂ alkyl)=N—CN, —(CH₂)ₜ—NH—C(NHC₁₋₂ alkyl)=CH—NO₂, —(CH₂)ₜ—C(NHC₁₋₂ alkyl)=N—CN, —(CH₂)ₜ—C(C₁₋₂ alkyl)=N—CN, —(CH₂)ₜ—C(C₁₋₂ alkyl)=N—O—(C₁₋₂ alkyl), ═N—O—C₁₋₂ alkyl, and a 5- to 6-membered heteroaryl; in some embodiments, R₁₂ is selected from cycloalkyl, cyclohexyl, phenyl, cyclopropyl, adamantyl, tetrahydropyranyl, piperidinyl, oxolanyl,

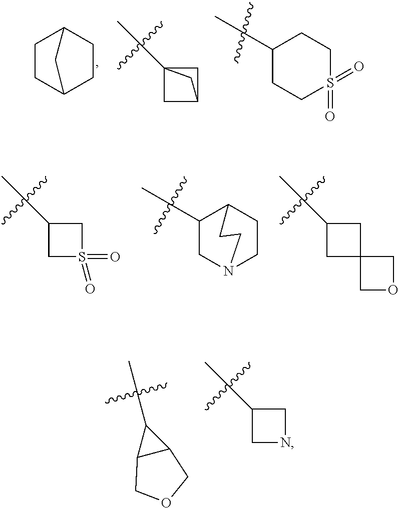

wherein the ring is optionally substituted with 1-3 groups selected from halogen, CN, OH, C₁₋₄ alkyl, hydroxy C₁₋₄ alkyl, and C₁₋₄ haloalkyl; in some embodiments, R₁₂ is selected from —C(CH₃)₂—CH₂—O—C(O)NH₂, —C(CH₃)₂—CH₂—NH—C(O)O(C₁₋₄ alkyl), —C(CH₃)₂—CH₂—O—C(O)NHCH₃, —CH(CH₃)—CH₂—O—C(O)NH₂, —CH(CH₃)—CH₂—NH—C(O)O(C₁₋₄ alkyl), and —CH(CH₃)—CH₂—O—C(O)NHCH₃;
Ra and Rb are each independently selected from H, halogen, C₁₋₄ alkyl, C₁₋₄ haloalkyl, C₁₋₄ alkoxy, halo C₁₋₄ alkoxy, or a 4- to 6-membered heterocycloalkyl containing 1-2 heteroatoms selected from N and O; or Ra and Rb are each independently selected from H, halogen, C₁₋₄ alkyl, C₁₋₄ haloalkyl, C₁₋₄ alkoxy, or halo C₁₋₄ alkoxy; or alternatively, Ra and Rb, together with the atom to which they are attached, form C₃₋₆ cycloalkyl;
R₁₃ is selected from a 7- to 11-membered spiro ring, a 4- to 10-membered fused ring, a 5- to 8-membered bridged heterocycle, a 4- to 6-membered monocyclic heterocycloalkyl containing S(O)₂ group, a 4-membered monocyclic heterocycloalkyl containing 1-3 heteroatoms selected from N, S, and O, and a 7-membered monocyclic heterocycloalkyl containing 1-3 heteroatoms selected from N, S, and O, wherein the spiro ring, fused ring, bridged heterocycle, and monocyclic heterocycloalkyl are optionally substituted with 1-3 groups selected from halogen, CN, ═O, OH, C₁₋₄ alkyl, hydroxy C₁₋₄ alkyl, and C₁₋₄ haloalkyl; in some embodiments, R₁₃ is selected from

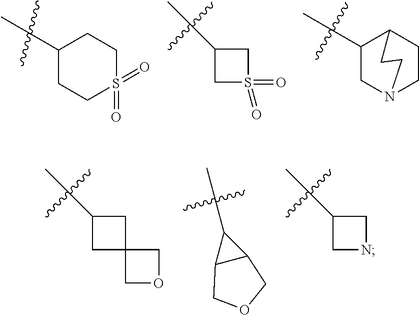

wherein the ring is optionally substituted with 1-3 groups selected from halogen, CN, OH, C₁₋₄ alkyl, hydroxy C₁₋₄ alkyl, and C₁₋₄ haloalkyl; R₁₄ is selected from —(CH₂)ₜ—O—C(O)NH₂, —(CH₂)ₜ—O—C(O)NHCH₃, and a 5- to 6-membered monocyclic heterocycloalkyl containing 1-3 heteroatoms selected from N, S, and O, wherein the monocyclic heterocycloalkyl is optionally substituted with 1-3 groups selected from halogen, CN, ═O, OH, C₁₋₄ alkyl, and C₁₋₄ haloalkyl; in some embodiments, R₁₄ is selected from —(CH₂)ₜ—O—C(O)NH₂ and a 5- to 6-membered monocyclic heterocycloalkyl containing 1-3 heteroatoms selected from N, S, and O; in some embodiments, R₁₄ is selected from —(CH₂)ₜ—O—C(O)NH₂,

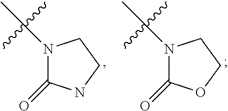 [SMILES: *N1CCNC1=O ; *N1CCOC1=O]

n and m are independently 0, 1, 2, or 3; in some embodiments, n is 0, 1, 2, or 3;
in some embodiments, m is 0, 1, or 2;
r is 1, 2, 3, or 4; in some embodiments, r is 1 or 2;
t is selected from 0, 1, 2, 3, or 4; in some embodiments, t is selected from 0, 1, or 2; and
p is selected from 0 or 1;
provided that
Cy1 is not

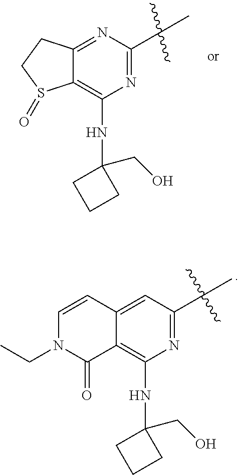 [SMILES: *c1nc2c(c(NC3(CO)CCC3)n1)S(=O)CC2 ; *c1cc2ccn(CC)c(=O)c2c(NC2(CO)CCC2)n1]

The present disclosure provides more specific technical solution 1, which relates to a compound represented by formula I, or a stereoisomer or a pharmaceutically acceptable salt thereof, wherein

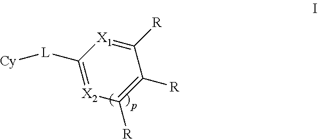

wherein when Cy is selected from Cy1, Cy2, Cy3, Cy8, and Cy10, L is -(L₁)n-(L₂)m-;

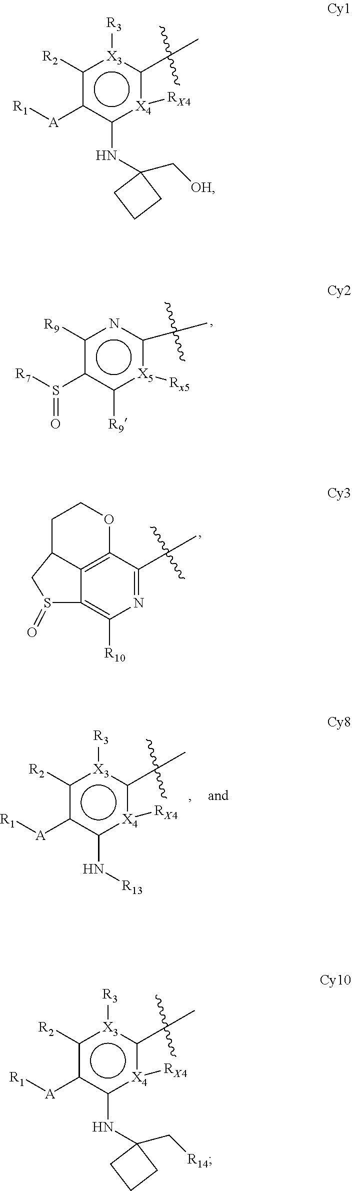

when Cy is selected from Cy4, Cy5, Cy6, and Cy11, L is -L₃-;

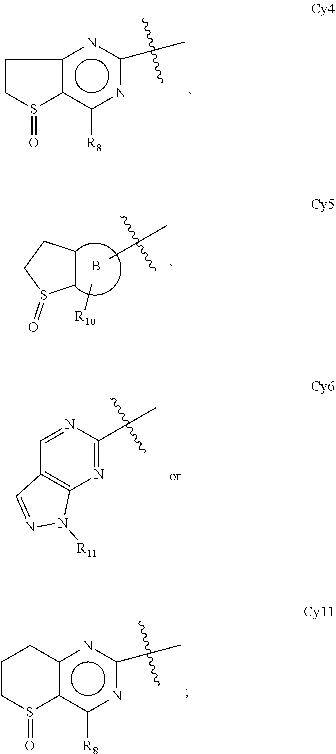

and
when Cy is selected from Cy7 and Cy9, L is -L₄-;

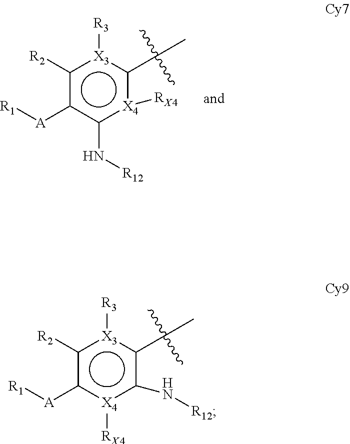

L₁ is —NR₄—, —CR₅=N—, —CR₅=CR₅—, or —CR₅R₅—;
L₂ is

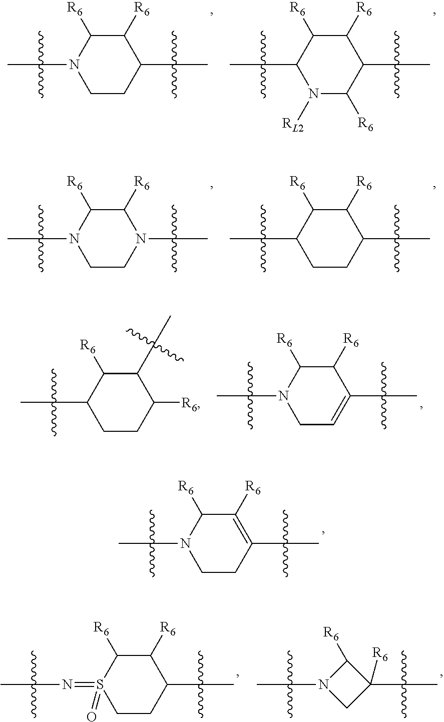

C₁₋₄ alkylene, C₂₋₄ alkenylene, C₂₋₄ alkynylene, CO, O, NR_{L2}, a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 10-membered fused heterocycloalkyl containing 1-3 heteroatoms selected from N, S, and O, a 6- to 10-membered bridged heterocycloalkyl containing 1-3 heteroatoms selected from N, S, and O, or a 7- to 12-membered spiro heterocycloalkyl containing 1-3 heteroatoms selected from N, S, and O, wherein the alkylene, alkenylene, alkynylene, monocyclic heterocycloalkyl, fused heterocycloalkyl, bridged heterocycloalkyl, and spiro heterocycloalkyl are optionally substituted with 1-3 groups selected from halogen, ═O, CN, C₁₋₄ alkyl, C₁₋₄ alkoxy, OH, and NH₂;
R_{L2} is H, C₁₋₄ alkyl, or C₃₋₆ cycloalkyl;
L₃ is heterocycloalkylene or heterocycloalkylene-(CH₂)ᵣ—, wherein the heterocycloalkylene is a 4- to 10-membered heterocycle containing 1-3 heteroatoms selected from N, S, and O and is optionally substituted with 1-3 R_{L3};
each R_{L3} is independently selected from halogen, ═O, CN, C₁₋₄ alkyl, or C₁₋₄ alkoxy, or R_{L3} and R_{X1}, together with the atom to which they are attached, form C₃₋₆ cycloalkyl or a 5- to 8-membered heterocycloalkyl containing 1-3 heteroatoms selected from N, S, and O;
L₄ is

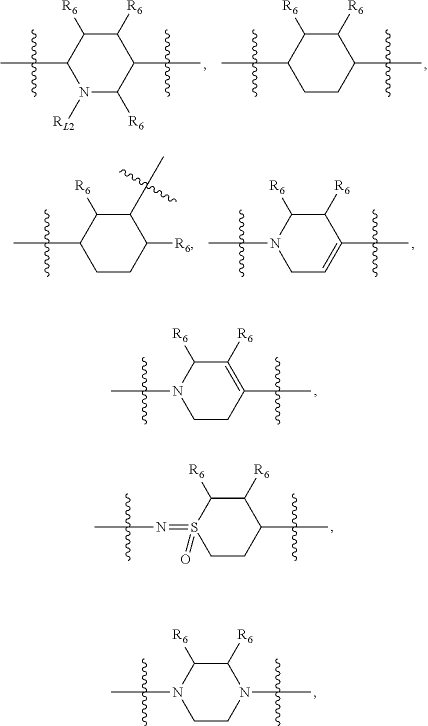

a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 6-membered heterocycloalkyl fused 5- to 6-membered heterocycloalkyl containing 1-3 heteroatoms selected from N, S, and O, a 5- to 6-membered heterocycloalkyl fused 5- to 6-membered cycloalkyl containing 1-3 heteroatoms selected from N, S, and O, or a 7- to 12-membered spiro heterocycloalkyl containing 1-3 heteroatoms selected from N, S, and O, wherein the monocyclic heterocycloalkyl, 5- to 6-membered heterocycloalkyl fused 5- to 6-membered heterocycloalkyl, 5- to 6-membered heterocycloalkyl fused 5- to 6-membered cycloalkyl, and spiro heterocycloalkyl are optionally substituted with 1-3 groups selected from halogen, ═O, CN, C₁₋₄ alkyl, C₁₋₄ alkoxy, OH, and NH₂; in some embodiments, L₄ is

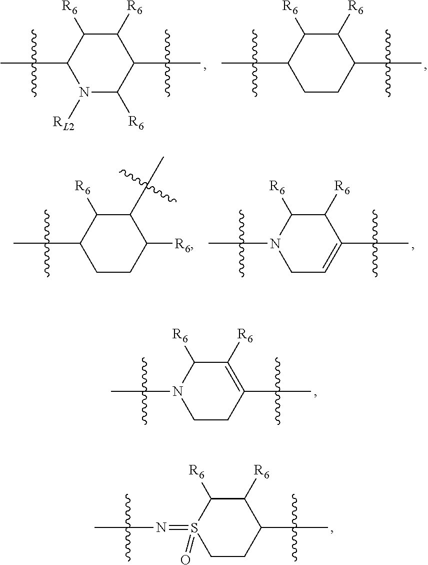

a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 6-membered heterocycloalkyl fused 5- to 6-membered heterocycloalkyl containing 1-3 heteroatoms selected from N, S, and O, a 5- to 6-membered heterocycloalkyl fused 5- to 6-membered cycloalkyl containing 1-3 heteroatoms selected from N, S, and O, or a 7- to 12-membered spiro heterocycloalkyl containing 1-3 heteroatoms selected from N, S, and O, wherein the monocyclic heterocycloalkyl, 5- to 6-membered heterocycloalkyl fused 5- to 6-membered heterocycloalkyl, 5- to 6-membered heterocycloalkyl fused 5- to 6-membered cycloalkyl, and spiro heterocycloalkyl are optionally substituted with 1-3 groups selected from halogen, ═O, CN, C₁₋₄ alkyl, C₁₋₄ alkoxy, OH, and NH₂; A is —S(O)—, —C(O)—, —B(OH)—, —S—, —S(═N)—CN, or —C(═N)—CN;
X₁ and X₂ are independently CR_{X1}, O, S, or N;
X₃, X₄, and X₅ are independently C or N;
ring B is heteroaryl or heterocycloalkyl fused heteroaryl, wherein the heteroaryl is a 5-membered heteroaryl containing 1-3 heteroatoms selected from N, S, and O, and the heterocycloalkyl is a 5- to 6-membered heterocycloalkyl containing 1-3 heteroatoms selected from N, S, and O;
each R_{X1} and R are independently H, halogen, C₁₋₄ alkyl, C₁₋₄ alkoxy, —N(CH₃)₂, —NH(CH₃), C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, O, and Si, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
or each R_{X1} and R are independently H, halogen, C₁₋₄ alkyl, C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, O, and Si, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, C₃₋₆ cycloalkyl, C₂₋₆ alkynyl, and NH₂;
or each R_{X1} and R are independently H, halogen, C₁₋₄ alkyl, C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, O, and Si, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
or each R_{X1} and R are independently H, halogen, C₁₋₄ alkyl, C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, or C₁₋₄ haloalkyl, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, and O, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₁ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₁ and R₂, together with the atom to which they are attached, form a 5- to 10-membered heterocycloalkyl or a 5- to 6-membered heteroaryl containing 1-3 heteroatoms selected from N, S, B, and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
R₂ is H, C₁₋₄ alkyl, or C₁₋₄ haloalkyl;
R₃ is H, halogen, C₁₋₄ alkyl, CN, OH, or absent, or R₃ and R₂, R₃ and R₆, R₃ and R₄, or R₃ and R₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent, or Rₓ₄ and R₄, Rₓ₄ and R₅, or Rₓ₄ and R₆, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₄ is H, C₁₋₄ alkyl, C₁₋₄ haloalkyl, or C₃₋₆ cycloalkyl;
R₅ is H, C₁₋₄ alkyl, C₁₋₄ haloalkyl, C₁₋₄ alkoxy, —NHC₁₋₄ alkyl, or C₃₋₆ cycloalkyl;
R₆ is H, halogen, ═O, CN, C₁₋₄ alkyl, or C₁₋₄ alkoxy;
Rₓ₅ is C₁₋₄ alkyl, CN, OH, or absent;
R₇ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₇ and R₉, together with the atom to which they are attached, form a 5- to 6-membered heterocycloalkyl containing 1-3 heteroatoms selected from N, S, and O, wherein the alkyl, haloalkyl, cycloalkyl, and heterocycloalkyl are optionally substituted with 1-3 groups selected from halogen, CN, C₁₋₄ alkyl, C₁₋₄ alkoxy, OH, and NH₂;
at least one group of R_{9′} and R₇ or R_{9′} and Rₓ₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the cycloalkyl, phenyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
R₈ is R_{8′}, —OR_{8′}, or —(CH₂)ᵣR_{8′};
R_{8′} is C₃₋₆ cycloalkyl, a 4- to 8-membered heterocycloalkyl containing 1-3 heteroatoms selected from N, S, O, Si, and P, a 5- to 6-membered heteroaryl containing 1-3 heteroatoms selected from N, S, and O, —N═S(═O)(C₁₋₄ alkyl)₂, or —N(C₃₋₆ cycloalkyl)C(O)NH(C₁₋₄ alkyl), wherein the cycloalkyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, and CN;
R₉ is H, C₁₋₄ alkyl, or C₁₋₄ haloalkyl;
each R₁₀ is independently R_{8′}, —OR_{8′}, —NHR_{8′}, or —(CH₂)ᵣR_{8′};
R₁₁ is C₃₋₆ cycloalkyl optionally substituted with 1-3 groups selected from halogen, CN, ═O, OH, C₁₋₄ alkyl, hydroxy C₁₋₄ alkyl, and C₁₋₄ haloalkyl;
R₁₂ is selected from a 4- to 10-membered heterocycloalkylene, a 3- to 10-membered cycloalkylene, a 6- to 10-membered arylene, a 5- to 10-membered heteroarylene, hydroxy-C₁₋₆ alkyl, C₁₋₄ alkyl-CN, —CRaRb=N—O—C₁₋₄ alkyl, —C(NRaRb)=N—CN, —C(C₁₋₄ alkyl)=N—CN, —C(NRaRb)=CRa—NO₂, —C(CH₃)₂—CH₂—O—C(O)NH₂, —C(CH₃)₂—CH₂—NH—C(O)O(C₁₋₄ alkyl), —C(CH₃)₂—CH₂—O—C(O)NHCH₃, —CH(CH₃)—CH₂—O—C(O)NH₂, —CH(CH₃)—CH₂—NH—C(O)O(C₁₋₄ alkyl), —CH(CH₃)—CH₂—O—C(O)NHCH₃, —C(O)—Ra, or —C(O)-(4- to 6-membered heterocycloalkyl), wherein the heterocycloalkylene, cycloalkylene, arylene, and heteroarylene are optionally further substituted with 1-3 groups selected from halogen, CN, ═O, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NRa—C(O)NH₂, —(CH₂)ₜ—NRa—C(═NH)NH₂, —(CH₂)ₜ—NRa—C(O)—O—(C₁₋₄ alkyl), —(CH₂)ₜ—NRa—C(C₁₋₄ alkyl)=N—CN, —(CH₂)ₜ—NRa—C(NRaRb)=N—CN, —(CH₂)ₜ—NRa—C(NRaRb)=CRa—NO₂, —(CH₂)ₜ—C(NRaRb)=N—CN, —(CH₂)ₜ—C(C₁₋₄ alkyl)=N—CN, —(CH₂)ₜ—C(C₁₋₄ alkyl)=N—O—(C₁₋₄ alkyl), ═N—O—C₁₋₄ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃;
or R₁₂ is selected from a 4- to 10-membered heterocycloalkylene, a 3- to 10-membered cycloalkylene, a 6- to 10-membered arylene, a 5- to 10-membered heteroarylene, hydroxy-C₁₋₆ alkyl, —CRaRb=N—O—C₁₋₄ alkyl, —C(NRaRb)=N—CN, —C(C₁₋₄ alkyl)=N—CN, —C(NRaRb)=CRa—NO₂, —C(CH₃)ₜ—CH₂—O—C(O)NH₂, —C(CH₃)ₜ—CH₂—NH—C(O)O(C₁₋₄ alkyl), —C(CH₃)ₜ—CH₂—O—C(O)NHCH₃, and —C(O)—Ra, or —C(O)-(4- to 6-membered heterocycloalkyl), wherein the heterocycloalkylene, cycloalkylene, arylene, and heteroarylene are optionally further substituted with 1-3 groups selected from halogen, CN, ═O, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NRa—C(O)NH₂, —(CH₂)ₜ—NRa—C(═NH)NH₂, —(CH₂)ₜ—NRa—C(O)—O—(C₁₋₄ alkyl), —(CH₂)ₜ—NRa—C(C₁₋₄ alkyl)=N—CN, —(CH₂)ₜ—NRa—C(NRaRb)=N—CN, —(CH₂)ₜ—NRa—C(NRaRb)=CRa—NO₂, —(CH₂)ₜ—C(NRaRb)=N—CN, —(CH₂)ₜ—C(C₁₋₄ alkyl)=N—CN, —(CH₂)ₜ—C(C₁₋₄ alkyl)=N—O—(C₁₋₄ alkyl), ═N—O—C₁₋₄ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃0.6 cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃;
Ra and Rb are each independently selected from H, halogen, C₁₋₄ alkyl, C₁₋₄ haloalkyl, C₁₋₄ alkoxy, halo C₁₋₄ alkoxy, or a 4- to 6-membered heterocycloalkyl containing 1-2 heteroatoms selected from N and O;
or alternatively, Ra and Rb, together with the atom to which they are attached, form C₃₋₆ cycloalkyl;
R₁₃ is selected from a 7- to 11-membered spiro ring, a 4- to 10-membered fused ring, a 5- to 8-membered bridged heterocycle, a 4- to 6-membered monocyclic heterocycloalkyl containing S(O)₂ group, a 4-membered monocyclic heterocycloalkyl containing 1-3 heteroatoms selected from N, S, and O, and a 7-membered monocyclic heterocycloalkyl containing 1-3 heteroatoms selected from N, S, and O, wherein the spiro ring, fused ring, bridged heterocycle, and monocyclic heterocycloalkyl are optionally substituted with 1-3 groups selected from halogen, CN, ═O, OH, C₁₋₄ alkyl, hydroxy C₁₋₄ alkyl, and C₁₋₄ haloalkyl;
R₁₄ is selected from —(CH₂)ₜ—O—C(O)NH₂, —(CH₂)ₜ—O—C(O)NHCH₃, and a 5- to 6-membered monocyclic heterocycloalkyl containing 1-3 heteroatoms selected from N, S, and O, wherein the monocyclic heterocycloalkyl is optionally substituted with 1-3 groups selected from halogen, CN, ═O, OH, C₁₋₄ alkyl, and C₁₋₄ haloalkyl;
n and m are independently 0, 1, 2, or 3;
r is selected from 1, 2, 3, or 4;
t is selected from 0, 1, 2, 3, or 4; and
p is selected from 0 or 1;
provided that
Cy1 is not

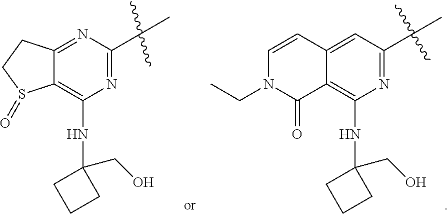 [SMILES: *c1nc2c(c(NC3(CO)CCC3)n1)S(=O)CC2 ; *c1cc2ccn(CC)c(=O)c2c(NC2(CO)CCC2)n1]

The present disclosure provides more specific technical solution 2, which relates to a compound represented by formula I, or a stereoisomer or a pharmaceutically acceptable salt thereof, wherein when Cy is selected from Cy1, Cy2, Cy3, Cy8, and Cy10, L is -(L₁)n-(L₂)m-;

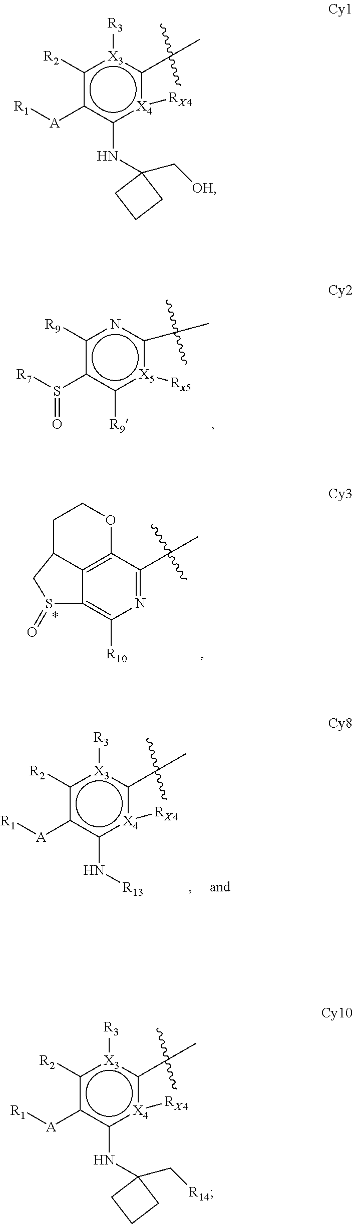

when Cy is selected from Cy4, Cy5, and Cy6, L is -L₃-;

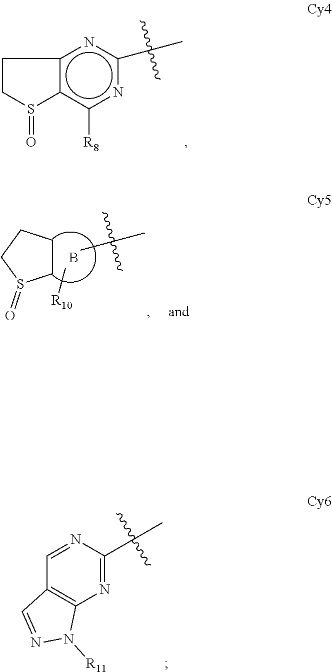

and
when Cy is selected from Cy7 and Cy9, L is -L₄-;

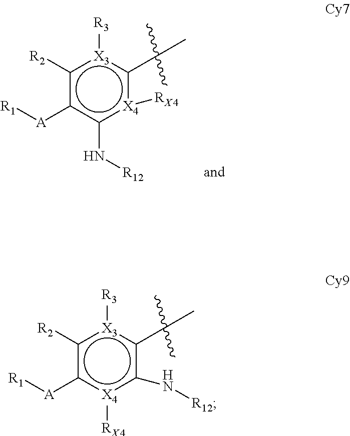

L₁ is —NR₄—, —CR₅=N—, —CR₅=CR₅—, or —CR₅R₅—;

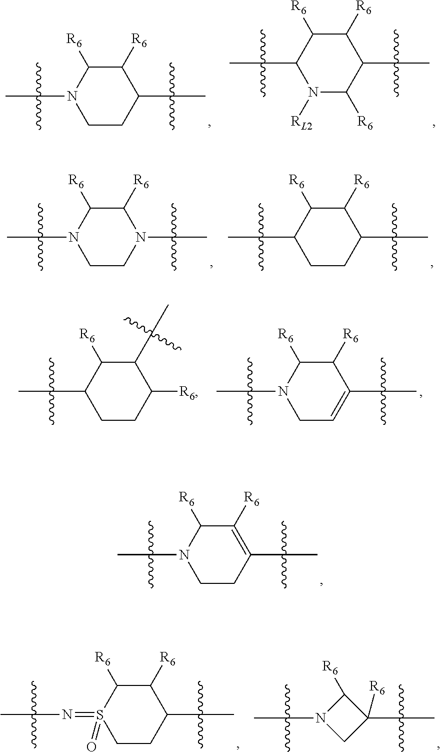

C₁₋₄ alkylene, C₂₋₄ alkenylene, C₂₋₄ alkynylene, CO, O, NR_{L2}, a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 10-membered fused heterocycloalkyl containing 1-3 heteroatoms selected from N, S, and O, a 6- to 10-membered bridged heterocycloalkyl containing 1-3 heteroatoms selected from N, S, and O, or a 7- to 12-membered spiro heterocycloalkyl containing 1-3 heteroatoms selected from N, S, and O, wherein the alkylene, alkenylene, alkynylene, monocyclic heterocycloalkyl, fused heterocycloalkyl, bridged heterocycloalkyl, and spiro heterocycloalkyl are optionally substituted with 1-3 groups selected from halogen, ═O, CN, C₁₋₄ alkyl, C₁₋₄ alkoxy, OH, and NH₂;
R_{L2} is H, C₁₋₄ alkyl, or C₃₋₆ cycloalkyl;
L₃ is heterocycloalkylene or heterocycloalkylene-(CH₂)ᵣ—, wherein the heterocycloalkylene is a 4- to 10-membered heterocycle containing 1-3 heteroatoms selected from N, S, and O and is optionally substituted with 1-3 R_{L3};
each R_{L3} is independently selected from halogen, ═O, CN, C₁₋₄ alkyl, or C₁₋₄ alkoxy, or R_{L3} and R_{X1}, together with the atom to which they are attached, form C₃₋₆ cycloalkyl or a 5- to 8-membered heterocycloalkyl containing 1-3 heteroatoms selected from N, S, and O;
L₄ is

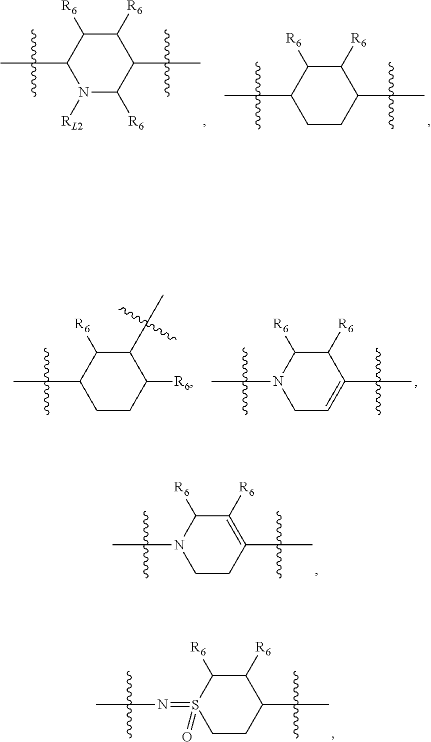

a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 6-membered heterocycloalkyl fused 5- to 6-membered heterocycloalkyl containing 1-3 heteroatoms selected from N, S, and O, a 5- to 6-membered heterocycloalkyl fused 5- to 6-membered cycloalkyl containing 1-3 heteroatoms selected from N, S, and O, or a 7- to 12-membered spiro heterocycloalkyl containing 1-3 heteroatoms selected from N, S, and O, wherein the monocyclic heterocycloalkyl, 5- to 6-membered heterocycloalkyl fused 5- to 6-membered heterocycloalkyl, 5- to 6-membered heterocycloalkyl fused 5- to 6-membered cycloalkyl, and spiro heterocycloalkyl are optionally substituted with 1-3 groups selected from halogen, ═O, CN, C₁₋₄ alkyl, C₁₋₄ alkoxy, OH, and NH₂;
A is —S(O)—, —C(O)—, —B(OH)—, —S—, —S(═N)—CN, or —C(═N)—CN;
X₁ and X₂ are independently CR_{X1}, O, S, or N;
X₃, X₄, and X₅ are independently C or N;
ring B is heteroaryl or heterocycloalkyl fused heteroaryl, wherein the heteroaryl is a 5-membered heteroaryl containing 1-3 heteroatoms selected from N, S, and O, and the heterocycloalkyl is a 5- to 6-membered heterocycloalkyl containing 1-3 heteroatoms selected from N, S, and O;
each R_{X1} and R are independently H, halogen, C₁₋₄ alkyl, C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, C₁₋₄ haloalkyl, or a 3- to 5-membered cycloalkyl, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, and O, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₁ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₁ and R₂, together with the atom to which they are attached, form a 5- to 10-membered heterocycloalkyl or a 5- to 6-membered heteroaryl containing 1-3 heteroatoms selected from N, S, B, and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
R₂ is H, C₁₋₄ alkyl, or C₁₋₄ haloalkyl;
R₃ is H, halogen, C₁₋₄ alkyl, CN, OH, or absent, or R₃ and R₂, R₃ and R₆, R₃ and R₄, or R₃ and R₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent, or Rₓ₄ and R₄, Rₓ₄ and R₅, or Rₓ₄ and R₆, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₄ is H, C₁₋₄ alkyl, C₁₋₄ haloalkyl, or C₃₋₆ cycloalkyl;
R₅ is H, C₁₋₄ alkyl, C₁₋₄ haloalkyl, C₁₋₄ alkoxy, —NHC₁₋₄ alkyl, or C₃₋₆ cycloalkyl;
R₆ is H, halogen, ═O, CN, C₁₋₄ alkyl, or C₁₋₄ alkoxy;
Rₓ₅ is C₁₋₄ alkyl, CN, OH, or absent;
R₇ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₇ and R₉, together with the atom to which they are attached, form a 5- to 6-membered heterocycloalkyl containing 1-3 heteroatoms selected from N, S, and O, wherein the alkyl, haloalkyl, cycloalkyl, and heterocycloalkyl are optionally substituted with 1-3 groups selected from halogen, CN, C₁₋₄ alkyl, C₁₋₄ alkoxy, OH, and NH₂;
at least one group of R_{9′} and R₇ or R_{9′} and Rₓ₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the cycloalkyl, phenyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
R₈ is R_{8′}, —OR_{8′}, or —(CH₂)ᵣR_{8′};
R_{8′} is C₃₋₆ cycloalkyl, a 4- to 8-membered heterocycloalkyl containing 1-3 heteroatoms selected from N, S, O, Si, and P, a 5- to 6-membered heteroaryl containing 1-3 heteroatoms selected from N, S, and O, or —N═S(═O)(C₁₋₄ alkyl)₂, or —N(C₃₋₆ cycloalkyl)C(O)NH(C₁₋₄ alkyl), wherein the cycloalkyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, and CN;
R₉ is H, C₁₋₄ alkyl, or C₁₋₄ haloalkyl;
each R₁₀ is independently R_{8′}, —OR_{8′}, —NHR_{8′}, or —(CH₂)ᵣR_{8′};
R₁₁ is C₃₋₆ cycloalkyl optionally substituted with 1-3 groups selected from halogen, CN, ═O, OH, C₁₋₄ alkyl, hydroxy C₁₋₄ alkyl, and C₁₋₄ haloalkyl;
R₁₂ is selected from a 4- to 10-membered heterocycloalkylene, a 3- to 10-membered cycloalkylene, a 6- to 10-membered arylene, a 5- to 10-membered heteroarylene, hydroxy-C₁₋₆ alkyl, —CRaRb=N—O—C₁₋₄ alkyl, —C(NRaRb)=N—CN, —C(C₁₋₄ alkyl)=N—CN, —C(NRaRb)=CRa—NO₂, —C(CH₃)ₜ—CH₂—O—C(O)NH₂, —C(O)—Ra, or —C(CH₃)ₜ—CH₂—O—C(O)NHCH₃, wherein the heterocycloalkylene, cycloalkylene, arylene, and heteroarylene are optionally further substituted with 1-3 groups selected from halogen, CN, ═O, OH, —SF₅, C₁₋₄ alkyl, hydroxy C₁₋₄ alkyl, C₁₋₄ haloalkyl, —(CH₂)ₜ—O—C(O)NH₂, —(CH₂)ₜ—NRa—C(O)NH₂, —(CH₂)ₜ—NRa—C(═NH)NH₂, —(CH₂)ₜ—NRa—C(O)—O—(C₁₋₄ alkyl), —(CH₂)ₜ—NRa—C(C₁₋₄ alkyl)=N—CN, —(CH₂)ₜ—NRa—C(NRaRb)=N—CN, —(CH₂)ₜ—NRa—C(NRaRb)=CRa-NO₂, —(CH₂)ₜ—C(NRaRb)=N—CN, —(CH₂)ₜ—C(C₁₋₄ alkyl)=N—CN, —(CH₂)ₜ—C(C₁₋₄ alkyl)=N—O—(C₁₋₄ alkyl), ═N—O—C₁₋₄ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃; or R₁₂ is selected from a 4- to 10-membered heterocycloalkylene, a 3- to 10-membered cycloalkylene, a 6- to 10-membered arylene, a 5- to 10-membered heteroarylene, hydroxy C₁₋₆ alkyl, —CRaRb=N—O—C₁₋₄ alkyl, —C(NRaRb)=N—CN, —C(C₁₋₄ alkyl)=N—CN, or —C(NRaRb)=CRa—NO₂, wherein the heterocycloalkylene, cycloalkylene, arylene, and heteroarylene are optionally further substituted with 1-3 groups selected from halogen, CN, ═O, OH, —SF₅, C₁₋₄ alkyl, hydroxy C₁₋₄ alkyl, C₁₋₄ haloalkyl, —(CH₂)ₜ—O—C(O)NH₂, —(CH₂)ₜ—NRa—C(O)NH₂, —(CH₂)ₜ—NRa—C(═NH)NH₂, —(CH₂)ₜ—NRa—C(O)—O—(C₁₋₄ alkyl), —(CH₂)ₜ—NRa—C(C₁₋₄ alkyl)=N—CN, —(CH₂)ₜ—NRa—C(NRaRb)=N—CN, —(CH₂)ₜ—NRa—C(NRaRb)=CRa—NO₂, —(CH₂)ₜ—C(NRaRb)=N—CN, —(CH₂)ₜ—C(C₁₋₄ alkyl)=N—CN, —(CH₂)ₜ—C(C₁₋₄ alkyl)=N—O—(C₁₋₄ alkyl), ═N—O—C₁₋₄ alkyl, and a 5- to 6-membered heteroaryl;
Ra and Rb are each independently selected from H, halogen, C₁₋₄ alkyl, C₁₋₄ haloalkyl, C₁₋₄ alkoxy, halo C₁₋₄ alkoxy, or a 4- to 6-membered heterocycloalkyl containing 1-2 heteroatoms selected from N and O; or alternatively, Ra and Rb, together with the atom to which they are attached, form C₃₋₆ cycloalkyl;
R₁₃ is selected from a 7- to 11-membered spiro ring, a 4- to 10-membered fused ring, a 5- to 8-membered bridged heterocycle, a 4- to 6-membered monocyclic heterocycloalkyl containing S(O)₂ group, a 4-membered monocyclic heterocycloalkyl containing 1-3 heteroatoms selected from N, S, and O, and a 7-membered monocyclic heterocycloalkyl containing 1-3 heteroatoms selected from N, S, and O, wherein the spiro ring, fused ring, bridged heterocycle, and monocyclic heterocycloalkyl are optionally substituted with 1-3 groups selected from halogen, CN, ═O, OH, C₁₋₄ alkyl, hydroxy C₁₋₄ alkyl, and C₁₋₄ haloalkyl;
R₁₄ is selected from —(CH₂)ₜ—O—C(O)NH₂, —(CH₂)ₜ—O—C(O)NHCH₃, and a 5- to 6-membered monocyclic heterocycloalkyl containing 1-3 heteroatoms selected from N, S, and O, wherein the monocyclic heterocycloalkyl is optionally substituted with 1-3 groups selected from halogen, CN, ═O, OH, C₁₋₄ alkyl, and C₁₋₄ haloalkyl;
n and m are independently 0, 1, 2, or 3;
r is selected from 1, 2, 3, or 4;
t is selected from 0, 1, 2, 3, or 4; and
P is selected from 0 or 1;
provided that
Cy1 is not

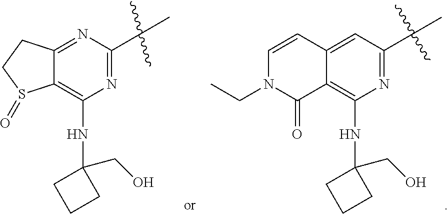 [SMILES: *c1nc2c(c(NC3(CO)CCC3)n1)S(=O)CC2 ; *c1cc2ccn(CC)c(=O)c2c(NC2(CO)CCC2)n1]

The present disclosure provides more specific technical solution 3, which relates to a compound represented by formula I, or a stereoisomer or a pharmaceutically acceptable salt thereof, wherein
when Cy is selected from Cy1, Cy2, Cy3, and Cy8, L is -(L₁)n-(L₂)m-;

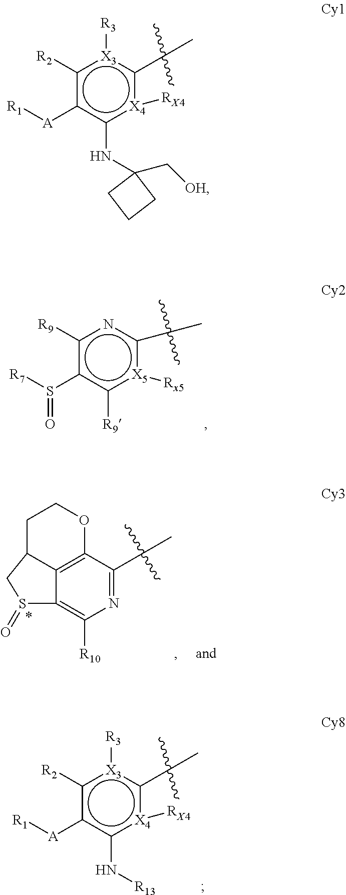

when Cy is selected from Cy4, Cy5, and Cy6, L is -L₃-;

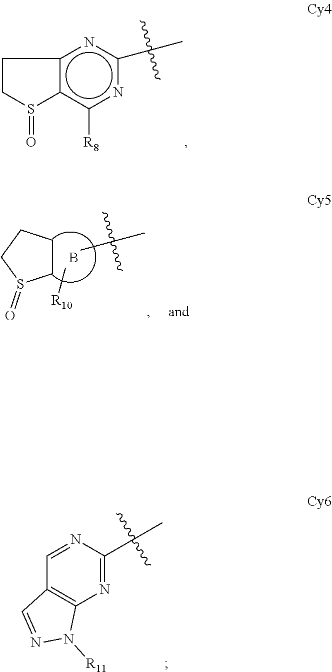

and
when Cy is selected from Cy7 and Cy9, L is -L₄-;

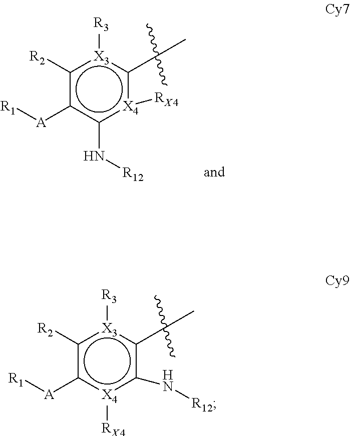

L₁ is —NR₄—, —CR₅=N—, —CR₅=CR₅—, or —CR5R₅—;
L₂ is

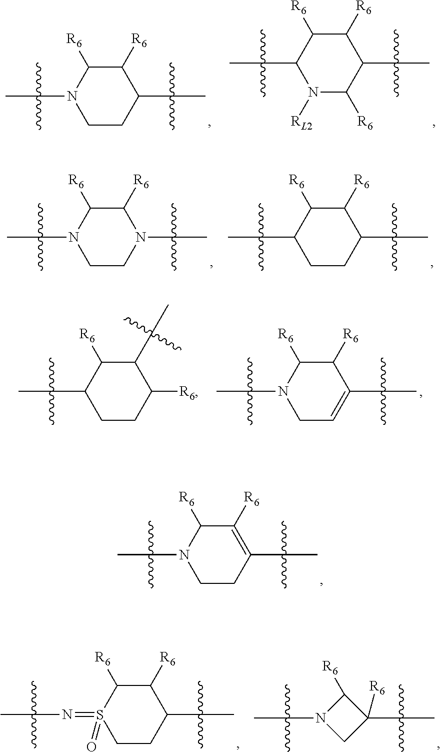

C₁₋₄ alkylene, C₂₋₄ alkenylene, C₂₋₄ alkynylene, CO, O, NR_{L2}, a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 10-membered fused heterocycloalkyl containing 1-3 heteroatoms selected from N, S, and O, a 6- to 10-membered bridged heterocycloalkyl containing 1-3 heteroatoms selected from N, S, and O, or a 7- to 12-membered spiro heterocycloalkyl containing 1-3 heteroatoms selected from N, S, and O, wherein the alkylene, alkenylene, alkynylene, monocyclic heterocycloalkyl, fused heterocycloalkyl, bridged heterocycloalkyl, and spiro heterocycloalkyl are optionally substituted with 1-3 groups selected from halogen, ═O, CN, C₁₋₄ alkyl, C₁₋₄ alkoxy, OH, and NH₂;
R_{L2} is H, C₁₋₄ alkyl, or C₃₋₆ cycloalkyl;
L₃ is heterocycloalkylene or heterocycloalkylene-(CH₂)ᵣ—, wherein the heterocycloalkylene is a 4- to 10-membered heterocycle containing 1-3 heteroatoms selected from N, S, and O and is optionally substituted with 1-3 R_{L3};
each R_{L3} is independently selected from halogen, ═O, CN, C₁₋₄ alkyl, or C₁₋₄ alkoxy, or R_{L3} and R_{X1}, together with the atom to which they are attached, form C₃₋₆ cycloalkyl or a 5- to 8-membered heterocycloalkyl containing 1-3 heteroatoms selected from N, S, and O;
L₄ is

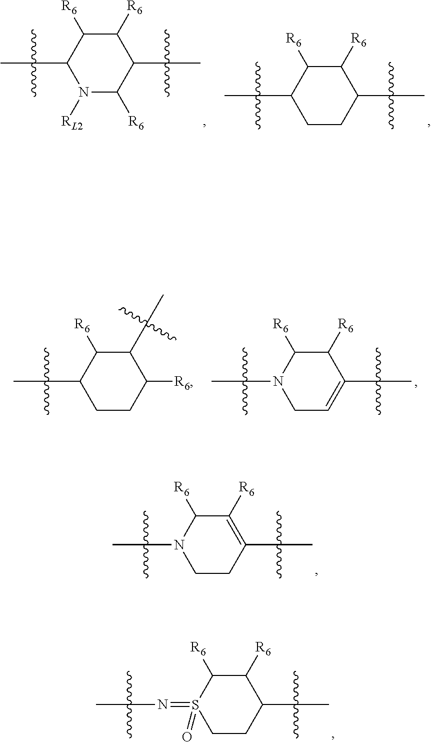

a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 6-membered heterocycloalkyl fused 5- to 6-membered heterocycloalkyl containing 1-3 heteroatoms selected from N, S, and O, a 5- to 6-membered heterocycloalkyl fused 5- to 6-membered cycloalkyl containing 1-3 heteroatoms selected from N, S, and O, or a 7- to 12-membered spiro heterocycloalkyl containing 1-3 heteroatoms selected from N, S, and O, wherein the monocyclic heterocycloalkyl, 5- to 6-membered heterocycloalkyl fused 5- to 6-membered heterocycloalkyl, 5- to 6-membered heterocycloalkyl fused 5- to 6-membered cycloalkyl, and spiro heterocycloalkyl are optionally substituted with 1-3 groups selected from halogen, ═O, CN, C₁₋₄ alkyl, C₁₋₄ alkoxy, OH, and NH₂;
A is —S(O)—, —C(O)—, —B(OH)—, —S—, —S(═N)—CN, or —C(═N)—CN;
X₁ and X₂ are independently CR_{X1} or N;
X₃, X₄, and X₅ are independently C or N;
ring B is heteroaryl or heterocycloalkyl fused heteroaryl, wherein the heteroaryl is a 5-membered heteroaryl containing 1-3 heteroatoms selected from N, S, and O, and the heterocycloalkyl is a 5- to 6-membered heterocycloalkyl containing 1-3 heteroatoms selected from N, S, and O;
each R_{X1} and R are independently H, halogen, C₁₋₄ alkyl, C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, or C₁₋₄ haloalkyl, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, and O, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₁ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₁ and R₂, together with the atom to which they are attached, form a 5- to 10-membered heterocycloalkyl or a 5- to 6-membered heteroaryl containing 1-3 heteroatoms selected from N, S, B, and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
R₂ is H, C₁₋₄ alkyl, or C₁₋₄ haloalkyl;
R₃ is H, halogen, C₁₋₄ alkyl, CN, OH, or absent, or R₃ and R₂, R₃ and R₆, R₃ and R₄, or R₃ and R₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent, or Rₓ₄ and R₄, Rₓ₄ and R₅, or Rₓ₄ and R₆, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₄ is H, C₁₋₄ alkyl, C₁₋₄ haloalkyl, or C₃₋₆ cycloalkyl;
R₅ is H, C₁₋₄ alkyl, C₁₋₄ haloalkyl, C₁₋₄ alkoxy, —NHC₁₋₄ alkyl, or C₃₋₆ cycloalkyl;
R₆ is H, halogen, ═O, CN, C₁₋₄ alkyl, or C₁₋₄ alkoxy;
Rₓ₅ is C₁₋₄ alkyl, CN, OH, or absent;
R₇ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₇ and R₉, together with the atom to which they are attached, form a 5- to 6-membered heterocycloalkyl containing 1-3 heteroatoms selected from N, S, and O, wherein the alkyl, haloalkyl, cycloalkyl, and heterocycloalkyl are optionally substituted with 1-3 groups selected from halogen, CN, C₁₋₄ alkyl, C₁₋₄ alkoxy, OH, and NH₂;
at least one group of R_{9′} and R₇ or R_{9′} and Rₓ₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the cycloalkyl, phenyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
R₈ is R_{8′}, —OR_{8′}, or —(CH₂)ᵣR_{8′};
R_{8′} is C₃₋₆ cycloalkyl, a 4- to 8-membered heterocycloalkyl containing 1-3 heteroatoms selected from N, S, O, Si, and P, a 5- to 6-membered heteroaryl containing 1-3 heteroatoms selected from N, S, and O, or —N═S(═O)(C₁₋₄ alkyl)₂, or —N(C₃₋₆ cycloalkyl)C(O)NH(C₁₋₄ alkyl), wherein the cycloalkyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, and CN;
R₉ is H, C₁₋₄ alkyl, or C₁₋₄ haloalkyl;
each R₁₀ is independently R_{8′}, —OR_{8′}, —NHR_{8′}, or —(CH₂)ᵣR_{8′};
R₁₁ is C₃₋₆ cycloalkyl optionally substituted with 1-3 groups selected from halogen, CN, ═O, OH, C₁₋₄ alkyl, hydroxy C₁₋₄ alkyl, and C₁₋₄ haloalkyl;
R₁₂ is selected from a 4- to 10-membered heterocycloalkylene, a 3- to 10-membered cycloalkylene, a 6- to 10-membered arylene, a 5- to 10-membered heteroarylene, hydroxy C₁₋₆ alkyl, —CRaRb=N—O—C₁₋₄ alkyl, —C(NRaRb)=N—CN, —C(C₁₋₄ alkyl)=N—CN, or —C(NRaRb)=CRa—NO₂, wherein the heterocycloalkylene, cycloalkylene, arylene, and heteroarylene are optionally further substituted with 1-3 groups selected from halogen, CN, ═O, OH, —SF₅, C₁₋₄ alkyl, hydroxy C₁₋₄ alkyl, C₁₋₄ haloalkyl, —(CH₂)ₜ—O—C(O)NH₂, —(CH₂)ₜ—NRa—C(O)NH₂, —(CH₂)ₜ—NRa—C(═NH)NH₂, —(CH₂)ₜ—NRa—C(O)—O—(C₁₋₄ alkyl), —(CH₂)ₜ—NRa—C(C₁₋₄ alkyl)=N—CN, —(CH₂)ₜ—NRa—C(NRaRb)=N—CN, —(CH₂)ₜ—NRa—C(NRaRb)=CRa—NO₂, —(CH₂)ₜ—C(NRaRb)=N—CN, —(CH₂)ₜ—C(C₁₋₄ alkyl)=N—CN, —(CH₂)ₜ—C(C₁₋₄ alkyl)=N—O—(C₁₋₄ alkyl), ═N—O—C₁₋₄ alkyl, and a 5- to 6-membered heteroaryl;
Ra and Rb are each independently selected from H, halogen, C₁₋₄ alkyl, C₁₋₄ haloalkyl, C₁₋₄ alkoxy, or halo C₁₋₄ alkoxy;
or alternatively, Ra and Rb, together with the atom to which they are attached, form C₃₋₆ cycloalkyl;
R₁₃ is selected from a 7- to 11-membered spiro ring, a 4- to 10-membered fused ring, a 5- to 8-membered bridged heterocycle, a 4- to 6-membered monocyclic heterocycloalkyl containing S(O)₂ group, a 4-membered monocyclic heterocycloalkyl containing 1-3 heteroatoms selected from N, S, and O, and a 7-membered monocyclic heterocycloalkyl containing 1-3 heteroatoms selected from N, S, and O, wherein the spiro ring, fused ring, bridged heterocycle, and monocyclic heterocycloalkyl are optionally substituted with 1-3 groups selected from halogen, CN, ═O, OH, C₁₋₄ alkyl, hydroxy C₁₋₄ alkyl, and C₁₋₄ haloalkyl;
n and m are independently 0, 1, 2, or 3;
r is selected from 1, 2, 3, or 4; and
t is selected from 0, 1, 2, 3, or 4;
provided that
Cy1 is not

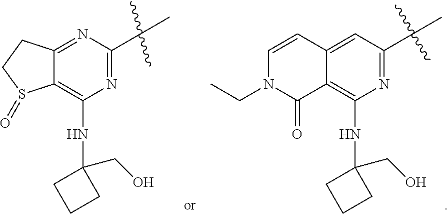 [SMILES: *c1nc2c(c(NC3(CO)CCC3)n1)S(=O)CC2 ; *c1cc2ccn(CC)c(=O)c2c(NC2(CO)CCC2)n1]

The present disclosure provides more specific technical solution 4, which relates to a compound represented by formula I, or a stereoisomer or a pharmaceutically acceptable salt thereof,
wherein when Cy is selected from Cy1, Cy2, Cy3, and Cy8, L is -(L₁)n-(L₂)m-;

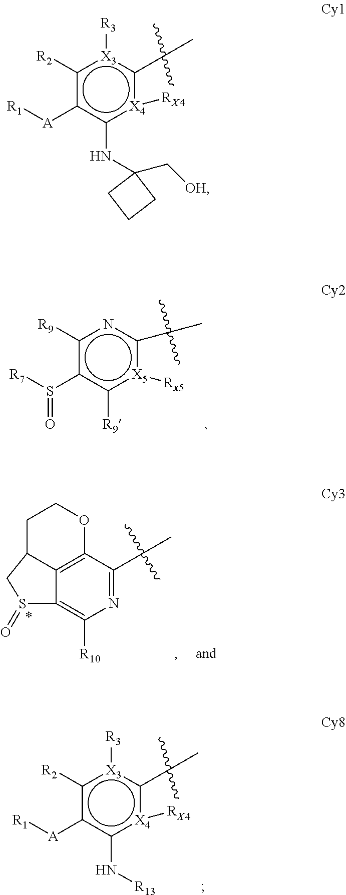

and
when Cy is selected from Cy4, Cy5, and Cy6, L is -L₃-;

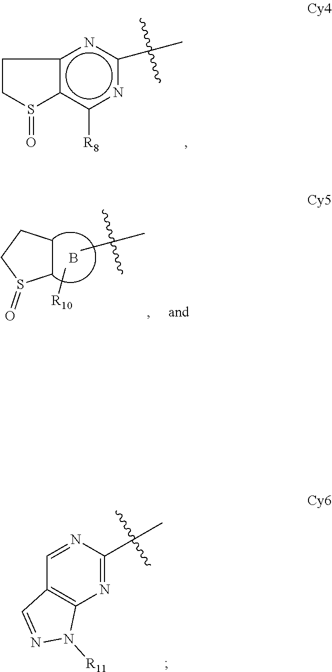

and
when Cy is Cy7, L is -L₄-;

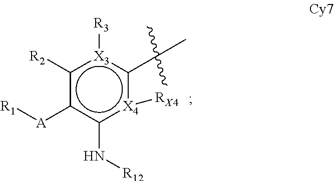

L₁ is —NR₄—, —CR₅=N—, —CR₅=CR₅—, or —CR₅R₅—;
L₂ is

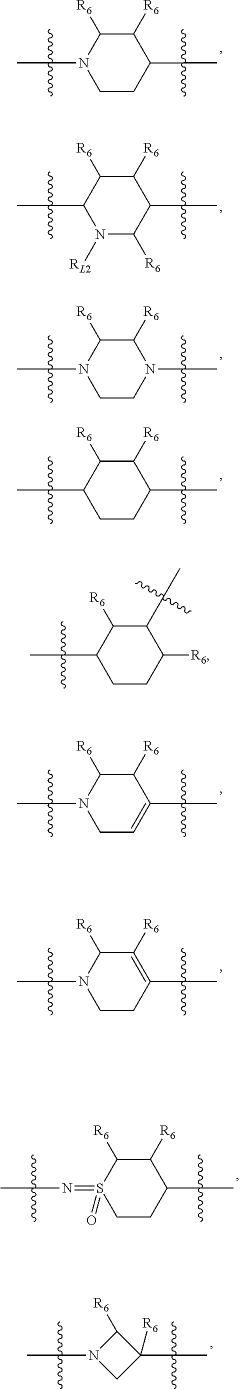

C₁₋₄ alkylene, C₂₋₄ alkenylene, C₂₋₄ alkynylene, CO, O, NR_{L2}, a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 10-membered fused heterocycloalkyl containing 1-3 heteroatoms selected from N, S, and O, a 6- to 10-membered bridged heterocycloalkyl containing 1-3 heteroatoms selected from N, S, and O, or a 7- to 12-membered spiro heterocycloalkyl containing 1-3 heteroatoms selected from N, S, and O, wherein the alkylene, alkenylene, alkynylene, monocyclic heterocycloalkyl, fused heterocycloalkyl, bridged heterocycloalkyl, and spiro heterocycloalkyl are optionally substituted with 1-3 groups selected from halogen, ═O, CN, C₁₋₄ alkyl, C₁₋₄ alkoxy, OH, and NH₂;
R_{L2} is H, C₁₋₄ alkyl, or C₃₋₆ cycloalkyl;
L₃ is heterocycloalkylene or heterocycloalkylene-(CH₂)ᵣ—, wherein the heterocycloalkylene is a 4- to 10-membered heterocycle containing 1-3 heteroatoms selected from N, S, and O and is optionally substituted with 1-3 R_{L3};
each R_{L3} is independently selected from halogen, ═O, CN, C₁₋₄ alkyl, or C₁₋₄ alkoxy, or R_{L3} and R_{X1}, together with the atom to which they are attached, form C₃₋₆ cycloalkyl or a 5- to 8-membered heterocycloalkyl containing 1-3 heteroatoms selected from N, S, and O;
L₄ is

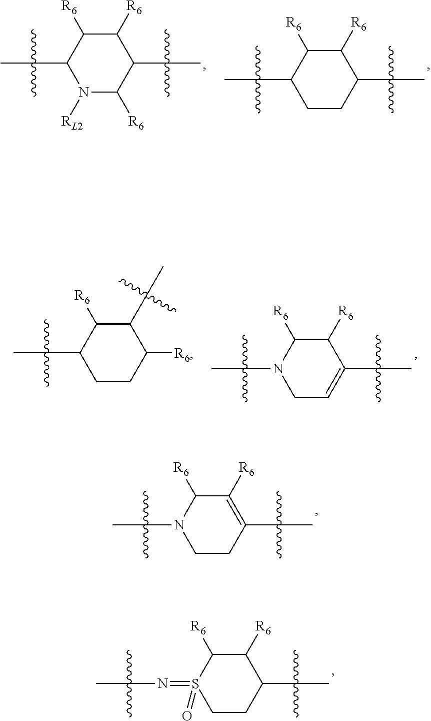

a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 6-membered cyclic heterocycloalkyl fused 5- to 6-membered cyclic heterocycloalkyl containing 1-3 heteroatoms selected from N, S, and O, a 5- to 6-membered cyclic heterocycloalkyl fused 5- to 6-membered cycloalkyl containing 1-3 heteroatoms selected from N, S, and O, or a 7- to 12-membered spiro heterocycloalkyl containing 1-3 heteroatoms selected from N, S, and O, wherein the monocyclic heterocycloalkyl, 5- to 6-membered cyclic heterocycloalkyl fused 5- to 6-membered cyclic heterocycloalkyl, 5- to 6-membered cyclic heterocycloalkyl fused 5- to 6-membered cycloalkyl, and spiro heterocycloalkyl are optionally substituted with 1-3 groups selected from halogen, ═O, CN, C₁₋₄ alkyl, C₁₋₄ alkoxy, OH, and NH₂;
A is —S(O)—, —C(O)—, —B(OH)—, —S—, —S(═N)—CN, or —C(═N)—CN;
X₁ and X₂ are independently CR_{X1} or N;
X₃, X₄, and X₅ are independently C or N;
ring B is heteroaryl or heterocycloalkyl fused heteroaryl, wherein the heteroaryl is a 5-membered heteroaryl containing 1-3 heteroatoms selected from N, S, and O, and the heterocycloalkyl is a 5- to 6-membered heterocycloalkyl containing 1-3 heteroatoms selected from N, S, and O;
each R_{X1} and R are independently H, halogen, C₁₋₄ alkyl, C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, or C₁₋₄ haloalkyl, or R_{X1} and R are independently 3- to 5-membered cycloalkyl, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, and O, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₁ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₁ and R₂, together with the atom to which they are attached, form a 5- to 10-membered heterocycloalkyl or a 5- to 6-membered heteroaryl containing 1-3 heteroatoms selected from N, S, B, and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
R₂ is H, C₁₋₄ alkyl, or C₁₋₄ haloalkyl;
R₃ is H, halogen, C₁₋₄ alkyl, CN, OH, or absent, or R₃ and R₂, R₃ and R₆, R₃ and R₄, or R₃ and R₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent, or Rₓ₄ and R₄, Rₓ₄ and R₅, or Rₓ₄ and R₆, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₄ is H, C₁₋₄ alkyl, C₁₋₄ haloalkyl, or C₃₋₆ cycloalkyl;
R₅ is H, C₁₋₄ alkyl, C₁₋₄ haloalkyl, C₁₋₄ alkoxy, —NHC₁₋₄ alkyl, or C₃₋₆ cycloalkyl;
R₆ is H, halogen, ═O, CN, C₁₋₄ alkyl, or C₁₋₄ alkoxy;
Rₓ₅ is C₁₋₄ alkyl, CN, OH, or absent;
R₇ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₇ and R₉, together with the atom to which they are attached, form a 5- to 6-membered heterocycloalkyl containing 1-3 heteroatoms selected from N, S, and O, wherein the alkyl, haloalkyl, cycloalkyl, and heterocycloalkyl are optionally substituted with 1-3 groups selected from halogen, CN, C₁₋₄ alkyl, C₁₋₄ alkoxy, OH, and NH₂;
at least one group of R_{9′} and R₇ or R_{9′} and Rₓ₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the cycloalkyl, phenyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
R₈ is R_{8′}, —OR_{8′}, or —(CH₂)ᵣR_{8′};
R_{8′} is C₃₋₆ cycloalkyl, a 4- to 8-membered heterocycloalkyl containing 1-3 heteroatoms selected from N, S, and O, or a 5- to 6-membered heteroaryl containing 1-3 heteroatoms selected from N, S, and O, or R_{8′} is —N(C₃₋₆ cycloalkyl)C(O)NH(C₁₋₄ alkyl), wherein the cycloalkyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, and CN;
R₉ is H, C₁₋₄ alkyl, or C₁₋₄ haloalkyl;
each R₁₀ is independently R_{8′}, —OR_{8′}, —NHR_{8′}, or —(CH₂)ᵣR_{8′};
R₁₁ is C₃₋₆ cycloalkyl optionally substituted with 1-3 groups selected from halogen, CN, ═O, OH, C₁₋₄ alkyl, hydroxy C₁₋₄ alkyl, and C₁₋₄ haloalkyl;
R₁₂ is selected from a 4- to 10-membered heterocycloalkylene, a 3- to 10-membered cycloalkylene, a 6- to 10-membered arylene, or a 5- to 10-membered heteroarylene, wherein the heterocycloalkylene, cycloalkylene, arylene, and heteroarylene are optionally further substituted with 1-3 groups selected from halogen, CN, ═O, OH, C₁₋₄ alkyl, hydroxy C₁₋₄ alkyl, and C₁₋₄ haloalkyl;
R₁₃ is selected from a 7- to 11-membered spiro ring, a 4- to 10-membered fused ring, a 5- to 8-membered bridged heterocycle, a 4- to 6-membered monocyclic heterocycloalkyl containing S(O)₂ group, a 4-membered monocyclic heterocycloalkyl containing 1-3 heteroatoms selected from N, S, and O, and a 7-membered monocyclic heterocycloalkyl containing 1-3 heteroatoms selected from N, S, and O, wherein the spiro ring, fused ring, bridged heterocycle, and monocyclic heterocycloalkyl are optionally substituted with 1-3 groups selected from halogen, CN, ═O, OH, C₁₋₄ alkyl, hydroxy C₁₋₄ alkyl, and C₁₋₄ haloalkyl;
n and m are independently 0, 1, 2, or 3; and
r is 1, 2, 3, or 4;
provided that
Cy1 is not

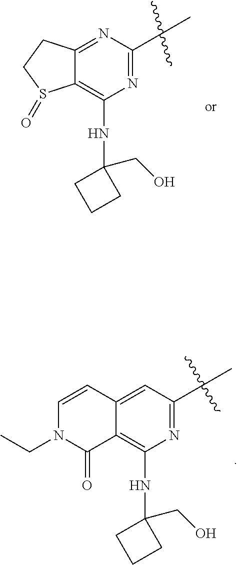 [SMILES: *c1nc2c(c(NC3(CO)CCC3)n1)S(=O)CC2 ; *c1cc2ccn(CC)c(=O)c2c(NC2(CO)CCC2)n1]

The present disclosure provides more specific technical solution 5, which relates to a compound represented by formula I, or a stereoisomer or a pharmaceutically acceptable salt thereof, wherein when Cy is selected from Cy1, Cy2, and Cy3, L is -(L₁)n-(L₂)m-;

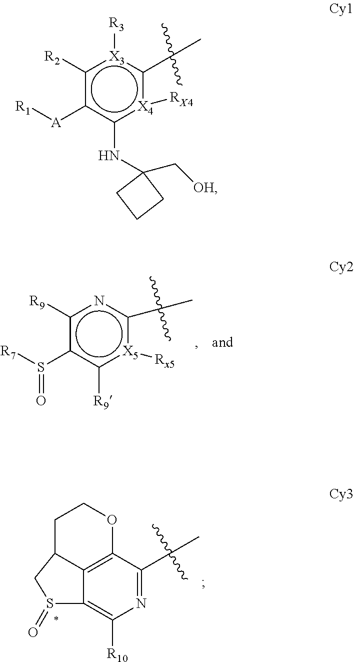

and
when Cy is selected from Cy4, Cy5, and Cy6, L is -L₃-;

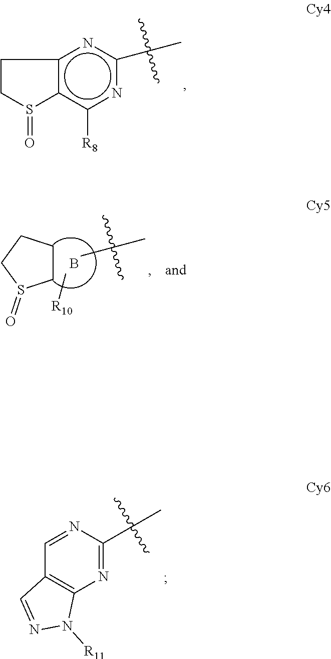

L₁ is —NR₄—, —CR₅=N—, —CR₅=CR₅—, or —CR₅R₅—;
L₂ is

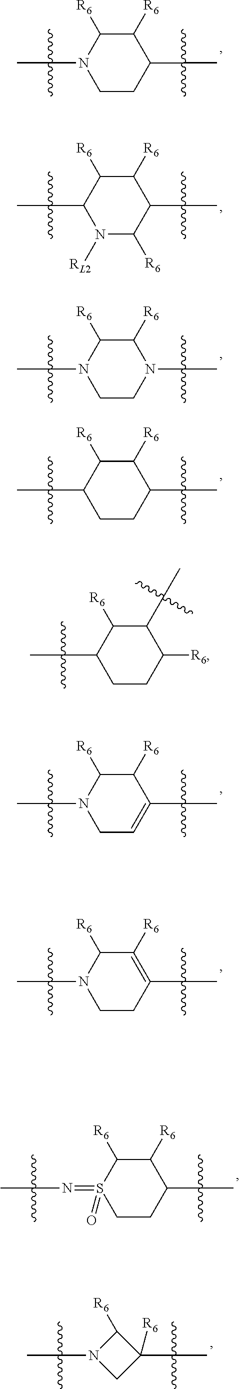

C₁₋₄ alkylene, C₂₋₄ alkenylene, C₂₋₄ alkynylene, CO, O, NR_{L2}, a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 10-membered fused heterocycloalkyl containing 1-3 heteroatoms selected from N, S, and O, a 6- to 10-membered bridged heterocycloalkyl containing 1-3 heteroatoms selected from N, S, and O, or a 7- to 12-membered spiro heterocycloalkyl containing 1-3 heteroatoms selected from N, S, and O, wherein the alkylene, alkenylene, alkynylene, monocyclic heterocycloalkyl, fused heterocycloalkyl, bridged heterocycloalkyl, and spiro heterocycloalkyl are optionally substituted with 1-3 groups selected from halogen, ═O, CN, C₁₋₄ alkyl, C₁₋₄ alkoxy, OH, and NH₂; or L₂ is

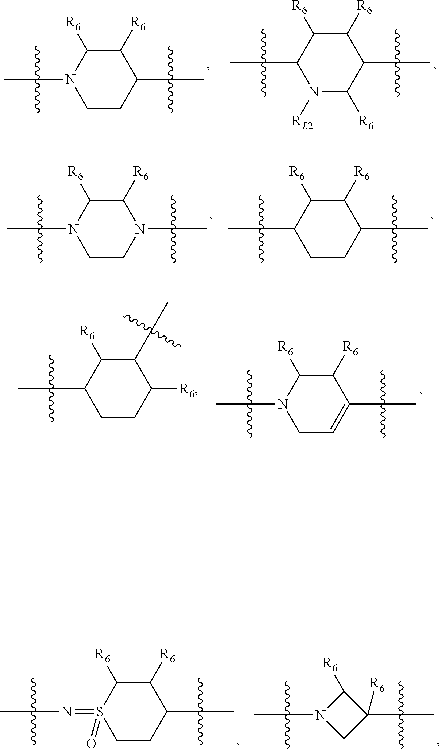

C₁₋₄ alkylene, C₂₋₄ alkenylene, C₂₋₄ alkynylene, CO, O, NR_{L2}, a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 10-membered fused heterocycloalkyl containing 1-3 heteroatoms selected from N, S, and O, a 6- to 10-membered bridged heterocycloalkyl containing 1-3 heteroatoms selected from N, S, and O, or a 7- to 12-membered spiro heterocycloalkyl containing 1-3 heteroatoms selected from N, S, and O, wherein the alkylene, alkenylene, alkynylene, monocyclic heterocycloalkyl, fused heterocycloalkyl, bridged heterocycloalkyl, and spiro heterocycloalkyl are optionally substituted with 1-3 groups selected from halogen, ═O, CN, C₁₋₄ alkyl, C₁₋₄ alkoxy, OH, and NH₂;
R_{L2} is H, C₁₋₄ alkyl, or C₃₋₆ cycloalkyl;
L₃ is heterocycloalkylene or heterocycloalkylene-(CH₂)ᵣ—, wherein the heterocycloalkylene is a 4- to 10-membered heterocycle containing 1-3 heteroatoms selected from N, S, and O and is optionally substituted with 1-3 R_{L3};
each R_{L3} is independently selected from halogen, ═O, CN, C₁₋₄ alkyl, or C₁₋₄ alkoxy, or R_{L3} and R_{X1}, together with the atom to which they are attached, form C₃₋₆ cycloalkyl or a 5- to 8-membered heterocycloalkyl containing 1-3 heteroatoms selected from N, S, and O;
A is —S(O)—, —C(O)—, —B(OH)—, —S—, —S(═N)—CN, or —C(═N)—CN;
X₁ and X₂ are independently CR_{X1} or N;
X₃, X₄, and X₅ are independently C or N;
ring B is heteroaryl or heterocycloalkyl fused heteroaryl, wherein the heteroaryl is a 5-membered heteroaryl containing 1-3 heteroatoms selected from N, S, and O, and the heterocycloalkyl is a 5- to 6-membered heterocycloalkyl containing 1-3 heteroatoms selected from N, S, and O;
each R_{X1} and R are independently H, halogen, C₁₋₄ alkyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, or C₁₋₄ haloalkyl, or C₁₋₄ haloalkyl, or R_{X1} and R are independently 3- to 5-membered cycloalkyl, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, and O, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₁ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₁ and R₂, together with the atom to which they are attached, form a 5- to 10-membered heterocycloalkyl or a 5- to 6-membered heteroaryl containing 1-3 heteroatoms selected from N, S, B, and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
R₂ is H, C₁₋₄ alkyl, or C₁₋₄ haloalkyl;
R₃ is H, halogen, C₁₋₄ alkyl, CN, OH, or absent, or R₃ and R₂, R₃ and R₆, R₃ and R₄, or R₃ and R₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent, or Rₓ₄ and R₄, Rₓ₄ and R₅, or Rₓ₄ and R₆, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₄ is H, C₁₋₄ alkyl, C₁₋₄ haloalkyl, or C₃₋₆ cycloalkyl;
R₅ is H, C₁₋₄ alkyl, C₁₋₄ haloalkyl, C₁₋₄ alkoxy, —NHC₁₋₄ alkyl, or C₃₋₆ cycloalkyl;
R₆ is H, halogen, ═O, CN, C₁₋₄ alkyl, or C₁₋₄ alkoxy;
Rₓ₅ is C₁₋₄ alkyl, CN, OH, or absent;
R₇ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₇ and R₉, together with the atom to which they are attached, form a 5- to 6-membered heterocycloalkyl containing 1-3 heteroatoms selected from N, S, and O, wherein the alkyl, haloalkyl, cycloalkyl, and heterocycloalkyl are optionally substituted with 1-3 groups selected from halogen, CN, C₁₋₄ alkyl, C₁₋₄ alkoxy, OH, and NH₂;
at least one group of R_{9′} and R₇ or R_{9′} and Rₓ₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the cycloalkyl, phenyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
R₈ is R_{8′}, —OR_{8′}, or —(CH₂)ᵣR_{8′};
R_{8′} is C₃₋₆ cycloalkyl, a 4- to 8-membered heterocycloalkyl containing 1-3 heteroatoms selected from N, S, and O, or a 5- to 6-membered heteroaryl containing 1-3 heteroatoms selected from N, S, and O, or R_{8′} is —N(C₃₋₆ cycloalkyl)C(O)NH(C₁₋₄ alkyl), wherein the cycloalkyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, and CN;
R₉ is H, C₁₋₄ alkyl, or C₁₋₄ haloalkyl;
each R₁₀ is independently R_{8′}, —OR_{8′}, —NHR_{8′}, or —(CH₂)ᵣR_{8′};
R₁₁ is C₃₋₆ cycloalkyl optionally substituted with 1-3 groups selected from halogen, CN, ═O, OH, C₁₋₄ alkyl, hydroxy C₁₋₄ alkyl, and C₁₋₄ haloalkyl;
n and m are independently 0, 1, 2, or 3; and
r is 1, 2, 3, or 4;
provided that
Cy1 is not

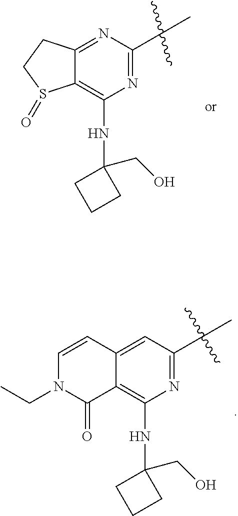 [SMILES: *c1nc2c(c(NC3(CO)CCC3)n1)S(=O)CC2 ; *c1cc2ccn(CC)c(=O)c2c(NC2(CO)CCC2)n1]

Solution 6 of the present disclosure relates to the compounds, or the stereoisomers or pharmaceutically acceptable salts thereof, as described above in solutions 1, 2, 3, 4, and 5, wherein ring B is pyrazolyl, imidazolyl, pyrrolyl, tetrahydropyrrolopyrrolyl, tetrahydropyrroloimidazolyl, or thienopyrrolyl;
R₁ is C₁₋₄ alkyl, or C₁₋₄ haloalkyl, or R₁ and R₂, together with the atom to which they are attached, form a 5- to 7-membered monocyclic heterocycloalkyl, a 6- to 8-membered fused heterocycloalkyl, a 7- to 9-membered spiro heterocycloalkyl, or a 5- to 6-membered heteroaryl containing 1-3 heteroatoms selected from N, S, B, and O, wherein the monocyclic heterocycloalkyl, fused heterocycloalkyl, spiro heterocycloalkyl, and heteroaryl are optionally substituted with 1-3 groups selected from halogen, ═O, CN, C₁₋₄ alkyl, C₁₋₄ alkoxy, OH, and NH₂;
R₂ is H, C₁₋₄ alkyl, or C₁₋₄ haloalkyl;
R₃ is H, halogen, C₁₋₄ alkyl, CN, OH, or absent;
or R₃ and R₂, together with the atom to which they are attached, form a 5- to 6-membered heterocycloalkyl or a 5- to 6-membered heteroaryl containing 1-3 heteroatoms selected from N, S, and O, wherein the heterocycloalkyl and heteroaryl are optionally substituted with 1-3 groups selected from halogen, ═O, CN, C₁₋₄ alkyl, C₁₋₄ alkoxy, OH, and NH₂;
or R₃ and R₄ or R₃ and R₅, together with the atom to which they are attached, form a 5- to 6-membered heterocycloalkyl or a 5- to 6-membered heteroaryl containing 1-3 heteroatoms selected from N, S, and O, or phenyl, wherein the heterocycloalkyl, phenyl, and heteroaryl are optionally substituted with 1-3 groups selected from halogen, ═O, CN, C₁₋₄ alkyl, C₁₋₄ alkoxy, OH, and NH₂;
or R₃ and R₆, together with the atom to which they are attached, form C₅₋₇ cycloalkyl, a 5- to 6-membered heterocycloalkyl, or a 5- to 6-membered heteroaryl containing 1-3 heteroatoms selected from N, S, and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent, or Rₓ₄ and R₄ or Rₓ₄ and R₅, together with the atom to which they are attached, form a 5- to 6-membered heteroaryl containing 1-3 heteroatoms selected from N, S, and O, wherein the heteroaryl is optionally substituted with 1-3 groups selected from halogen, ═O, CN, C₁₋₄ alkyl, C₁₋₄ alkoxy, OH, and NH₂;
R₇ is C₁₋₄ alkyl, or C₁₋₄ haloalkyl, or R₇ and R₉, together with the atom to which they are attached, form a 5- to 6-membered heterocycloalkyl containing 1-3 heteroatoms selected from N, S, and O, wherein the heterocycloalkyl is optionally substituted with 1-3 groups selected from halogen, CN, C₁₋₄ alkyl, C₁₋₄ alkoxy, OH, and NH₂;
at least one group of R_{9′} and R₇ or R_{9′} and Rₓ₅, together with the atom to which they are attached, form a 5- to 7-membered monocyclic heterocycloalkyl, a 7- to 10-membered spiro heterocycloalkyl, or a 5- to 6-membered heteroaryl containing 1-3 heteroatoms selected from N, S, and O, wherein the monocyclic heterocycloalkyl, spiro heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′}; and
r is 1 or 2.

Solution 7 of the present disclosure relates to the compounds as described in solutions 1-6, or stereoisomers or pharmaceutically acceptable salts thereof, wherein each L₂ is independently selected from

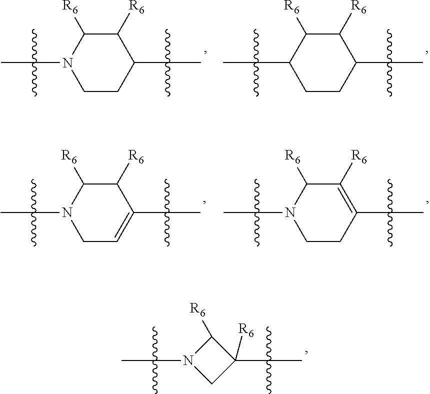

CO, O, NR_{L2}, C₁₋₄ alkylene, C₂₋₄ alkenylene, C₂₋₄ alkynylene,

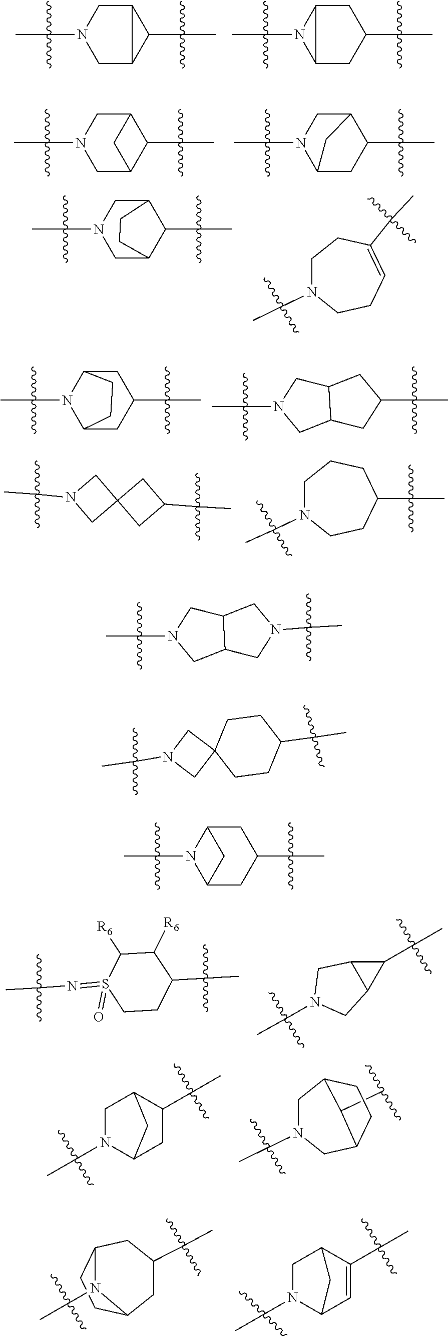

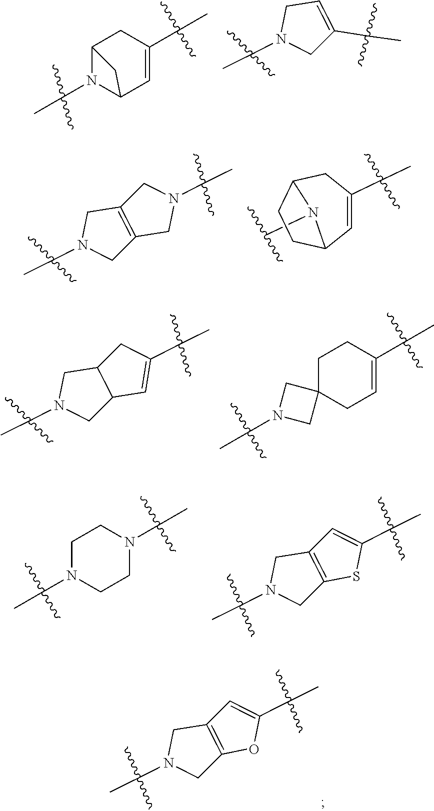

R_{L2} is H, C₁₋₄ alkyl, or C₃₋₆ cycloalkyl;
L₃ is selected from

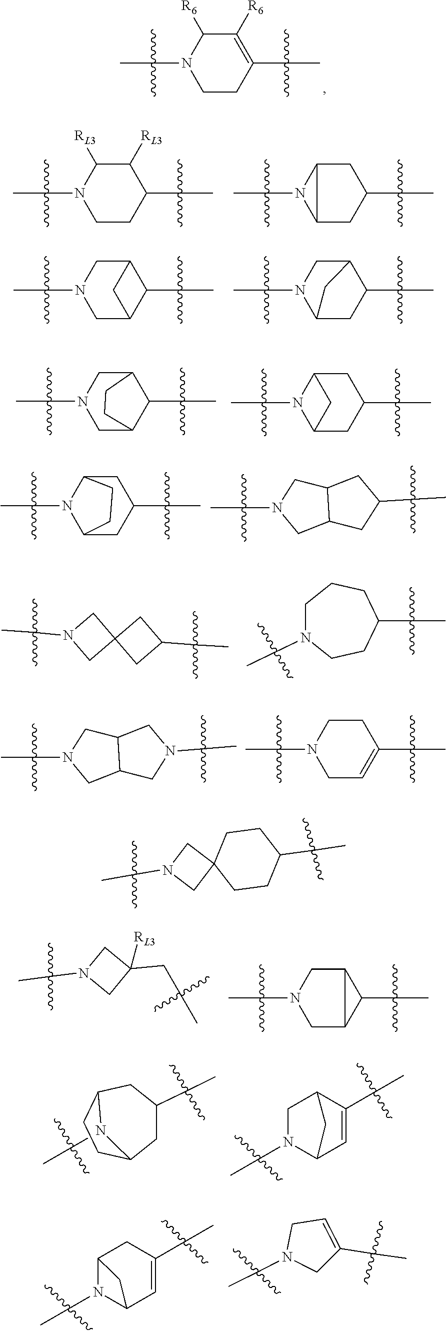

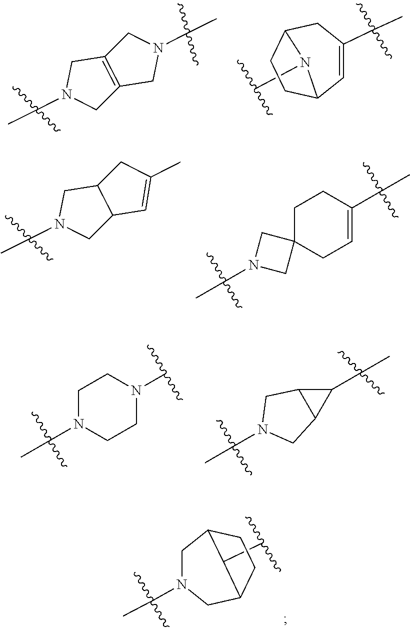

each R_{L3} is independently selected from halogen, ═O, CN, C₁₋₄ alkyl, or C₁₋₄ alkoxy, or R_{L3} and R_{X1}, together with the atom to which they are attached, form C₃₋₆ cycloalkyl or a 5- to 6-membered heterocycloalkyl containing 1-3 heteroatoms selected from N, S, and O;
L₄ is selected from

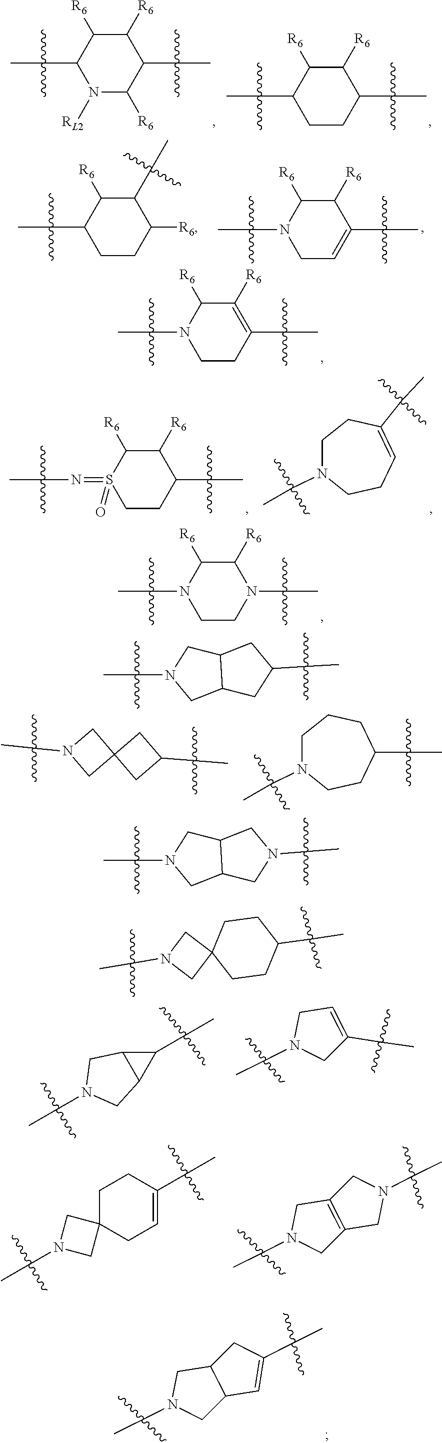

in some embodiments, L₄ is selected from

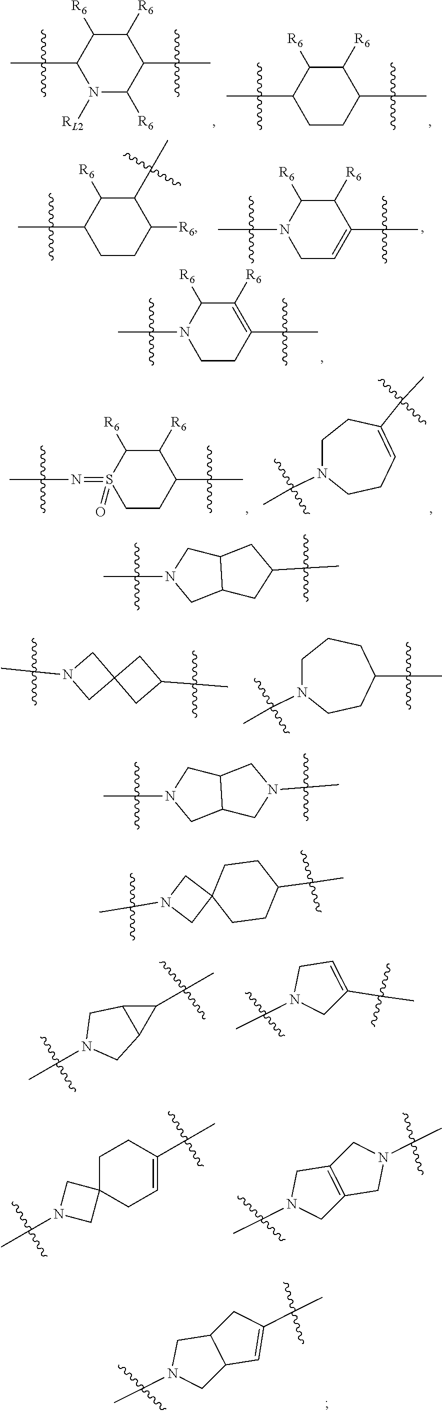

A is —S(O)—, —C(O)—, —B(OH)—, —S—, or —S(═N)—CN;
Cy5 is selected from

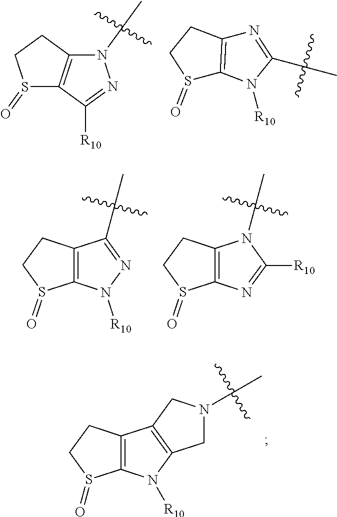

each R_{X1} and R are independently H, halogen, C₁₋₄ alkyl, C₁₋₄ alkoxy, —N(CH₃)₂, —NH(CH₃), C₂₋₄ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₆ cycloalkyl, or a 5- to 6-membered heterocycloalkyl containing 1-3 heteroatoms selected from N, S, O, and Si, wherein the cycloalkyl and heterocycloalkyl are optionally substituted with 1-3 groups selected from halogen, ═O, CN, C₁₋₄ alkyl, C₁₋₄ alkoxy, OH, and NH₂;
or each R_{X1} and R are independently H, halogen, C₁₋₄ alkyl, C₂₋₄ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₆ cycloalkyl or a 5- to 6-membered heterocycloalkyl containing 1-3 heteroatoms selected from N, S, O, and Si, wherein the cycloalkyl and heterocycloalkyl are optionally substituted with 1-3 groups selected from halogen, ═O, CN, C₁₋₄ alkyl, C₁₋₄ alkoxy, OH, C₃₋₆ cycloalkyl, C₂₋₆ alkynyl, and NH₂;
or each R_{X1} and R are independently H, halogen, C₁₋₄ alkyl, C₂₋₄ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₆ cycloalkyl or a 5- to 6-membered heterocycloalkyl containing 1-3 heteroatoms selected from N, S, O, and Si, wherein the cycloalkyl and heterocycloalkyl are optionally substituted with 1-3 groups selected from halogen, ═O, CN, C₁₋₄ alkyl, C₁₋₄ alkoxy, OH, and NH₂;
or each R_{X1} and R are independently H, halogen, C₁₋₄ alkyl, C₂₋₄ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, or C₁₋₄ haloalkyl, or two R on adjacent ring atoms, R and R_{X1}, or R_{X1} and R₆, together with the atom to which they are attached, form C₃₋₆ cycloalkyl or a 5- to 6-membered heterocycloalkyl containing 1-3 heteroatoms selected from N, S, and O, wherein the cycloalkyl and heterocycloalkyl are optionally substituted with 1-3 groups selected from halogen, ═O, CN, C₁₋₄ alkyl, C₁₋₄ alkoxy, OH, and NH₂;
R₁ is C₁₋₄ alkyl or C₁₋₄ haloalkyl;
or R₁ and R₂, together with the atom to which they are attached, form a ring selected from:

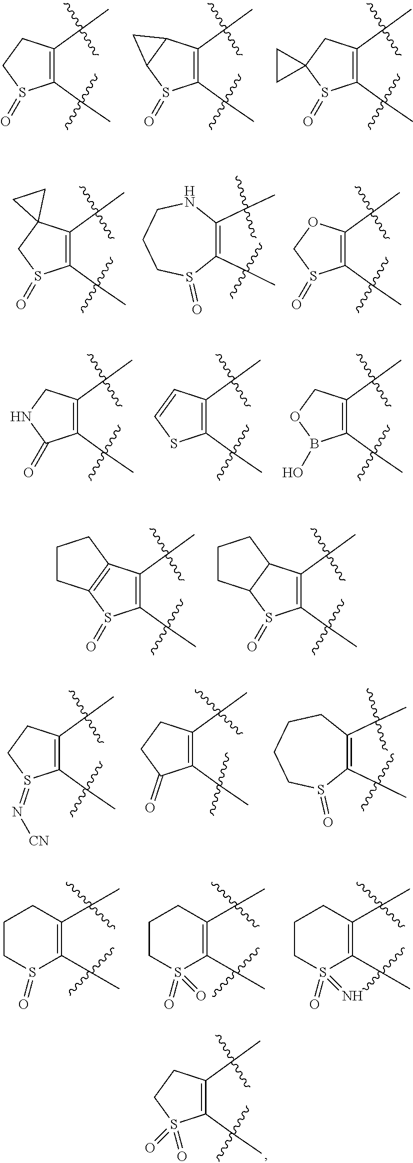

optionally substituted with 1-3 groups selected from halogen, ═O, CN, C₁₋₄ alkyl, C₁₋₄ alkoxy, OH, and NH₂;
R₃ is H, halogen, C₁₋₄ alkyl, CN, OH, or absent;
or R₃ and R₂, together with the atom to which they are attached, form a ring selected from:

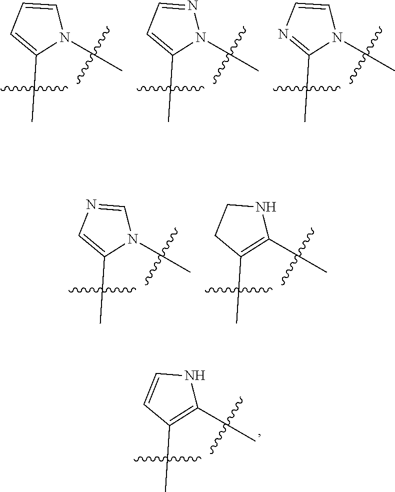 [SMILES: *c1cccn1* ; *c1ccnn1* ; *c1nccn1* ; *c1cncn1* ; *C1=C(*)NCC1 ; *c1cc[nH]c1*]

optionally substituted with 1-3 groups selected from halogen, ═O, CN, C₁₋₄ alkyl, C₁₋₄ alkoxy, OH, and NH₂;
or R₃ and R₄ or R₃ and R₅, together with the atom to which they are attached, form a 5- to 6-membered heterocycloalkyl or a 5- to 6-membered heteroaryl containing 1-3 heteroatoms selected from N, S, and O, or phenyl, wherein the heterocycloalkyl, phenyl, and heteroaryl are optionally substituted with 1-3 groups selected from halogen, ═O, CN, C₁₋₄ alkyl, C₁₋₄ alkoxy, OH, and NH₂;
or R₃ and R₆, together with the atom to which they are attached, form C₅₋₆ cycloalkyl, or a 5- to 6-membered heterocycloalkyl containing 1-3 heteroatoms selected from N, S, and O, optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent; or Rₓ₄ and R₄ or Rₓ₄ and R₅, together with the atom to which they are attached, form imidazolyl, pyrazolyl, pyrrolyl, thienyl, or thiazolyl, optionally substituted with 1-3 groups selected from halogen, ═O, CN, C₁₋₄ alkyl, C₁₋₄ alkoxy, OH, and NH₂;
R₄ is H, C₁₋₄ alkyl, or C₁₋₄ haloalkyl;
R₅ is H, C₁₋₄ alkyl, C₁₋₄ haloalkyl, C₁₋₄ alkoxy, or —NHC₁₋₄ alkyl;
R₆ is H, halogen, ═O, CN, C₁₋₄ alkyl, or C₁₋₄ alkoxy;
R₇ is C₁₋₄ alkyl, or C₁₋₄ haloalkyl, or R₇ and R₉, together with the atom to which they are attached, form a ring selected from:

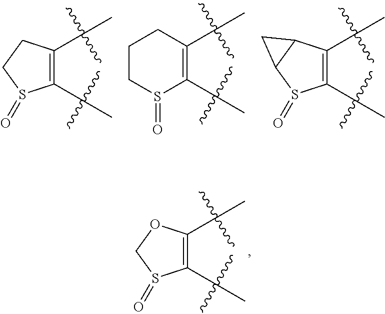 [SMILES: *C1=C(*)S(=O)CC1 ; *C1=C(*)S(=O)CCC1 ; *C1=C(*)S(=O)C2CC12 ; *C1=C(*)S(=O)CO1]

wherein the ring is optionally substituted with 1-3 groups selected from halogen, CN, C₁₋₄ alkyl, C₁₋₄ alkoxy, OH, and NH₂;
or R_{9′} and R₇, together with the atom to which they are attached, form a ring selected from:

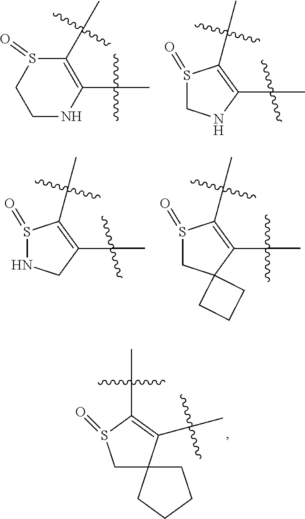 [SMILES: *C1=C(*)S(=O)CCN1 ; *C1=C(*)S(=O)CN1 ; *C1=C(*)S(=O)NC1 ; *C1=C(*)C2(CCC2)CS1=O ; *C1=C(*)C2(CCCC2)CS1=O]

optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
or R_{9′} and Rₓ₅, together with the atom to which they are attached, form

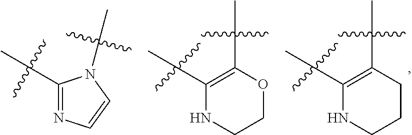 [SMILES: *c1nccn1* ; *C1=C(*)OCCN1 ; *C1=C(*)NCCC1]

optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
provided that at least one group of R_{9′} and R₇ or R_{9′} and Rₓ₅ form a ring;
R₈ is R_{8′}, —OR_{8′}, or —(CH₂)ᵣR_{8′};
R_{8′} is C₃₋₆ cycloalkyl, a 4- to 8-membered heterocycloalkyl containing 1-3 heteroatoms selected from N, S, O, Si, and P, a 5- to 6-membered heteroaryl containing 1-3 heteroatoms selected from N, S, and O, —N═S(═O)(C₁₋₄ alkyl)₂, or —N(C₃₋₆ cycloalkyl)C(O)NH(C₁₋₄ alkyl), wherein the cycloalkyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, and CN;
R₉ is H or C₁₋₄ alkyl;
each R₁₀ is independently R_{8′}, —OR_{8′}, —NHR_{8′}, or —(CH₂)ᵣR_{8′};
R₁₁ is C₃₋₆ cycloalkyl optionally substituted with 1-3 groups selected from halogen, CN, ═O, OH, C₁₋₄ alkyl, hydroxy C₁₋₄ alkyl, and C₁₋₄ haloalkyl;
R₁₂ is selected from cyclobutyl, cyclopentyl, cyclohexyl, phenyl, cyclopropyl, adamantyl, tetrahydropyranyl, piperidinyl, oxolanyl, oxanyl,

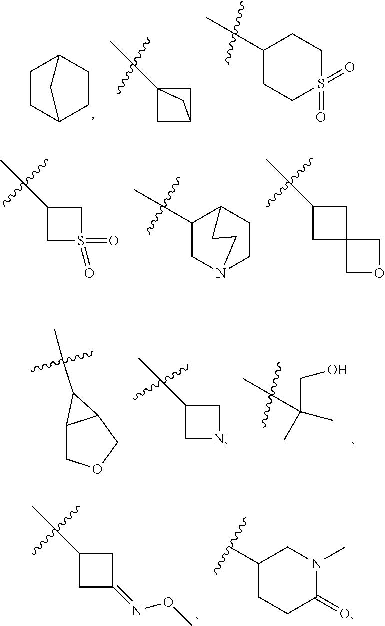

—C(NHC₁₋₂ alkyl)=N—CN, —C(C₁₋₂ alkyl)=N—CN, —C(NHC₁₋₂ alkyl)=CH—NO₂, —C(CH₃)ₜ—CH₂—NH—C(O)O(CH₃), —C(CH₃)ₜ—CH₂—O—C(O)NH₂, —C(O)—Ra, or —C(CH₃)ₜ—CH₂—O—C(O)NHCH₃; wherein the ring is optionally substituted with 1-3 groups selected from halogen, CN, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NH—C(O)NH₂, —(CH₂)ᵣ—NH—C(═NH)NH₂, —(CH₂)ₜ—NH—C(O)—O—(C₁₋₂ alkyl), —(CH₂)ₜ—NH—C(C₁₋₂ alkyl)=N—CN, —(CH₂)ₜ—NH—C(NHC₁₋₂ alkyl)=N—CN, —(CH₂)ₜ—NH—C(NHC₁₋₂ alkyl)=CH—NO₂, —(CH₂)ₜ—C(NHC₁₋₂ alkyl)=N—CN, —(CH₂)ₜ—C(C₁₋₂ alkyl)=N—CN, —(CH₂)ₜ—C(C₁₋₂ alkyl)=N—O—(C₁₋₂ alkyl), =N—O—C₁₋₂ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃;
in some embodiments, R₁₂ is selected from cyclobutyl, cyclopentyl, cyclohexyl, phenyl, cyclopropyl, adamantyl, tetrahydropyranyl, piperidinyl, oxolanyl, oxanyl,

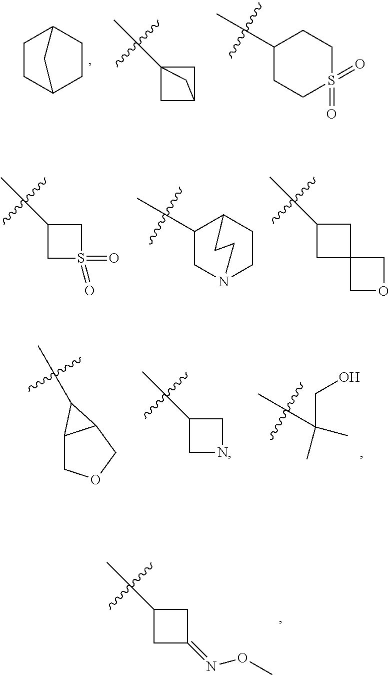

—C(NHC₁₋₂ alkyl)=N—CN, —C(C₁₋₂ alkyl)=N—CN, —C(NHC₁₋₂ alkyl)=CH—NO₂, —C(CH₃)ₜ—CH₂—NH—C(O)O(CH₃), —C(CH₃)ₜ—CH₂—O—C(O)NH₂, —C(O)—Ra, or —C(CH₃)ₜ—CH₂—O—C(O)NHCH₃; wherein the ring is optionally substituted with 1-3 groups selected from halogen, CN, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NH—C(O)NH₂, —(CH₂)ₜ—NH—C(═NH)NH₂, —(CH₂)ₜ—NH—C(O)—O—(C₁₋₂ alkyl), —(CH₂)ₜ—NH—C(C₁₋₂ alkyl)=N—CN, —(CH₂)ₜ—NH—C(NHC₁₋₂ alkyl)=N—CN, —(CH₂)ₜ—NH—C(NHC₁₋₂ alkyl)=CH—NO₂, —(CH₂)ₜ—C(NHC₁₋₂ alkyl)=N—CN, —(CH₂)ₜ—C(C₁₋₂ alkyl)=N—CN, —(CH₂)ₜ—C(C₁₋₂ alkyl)=N—O—(C₁₋₂ alkyl), ═N—O—C₁₋₂ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃;
R₁₃ is selected from

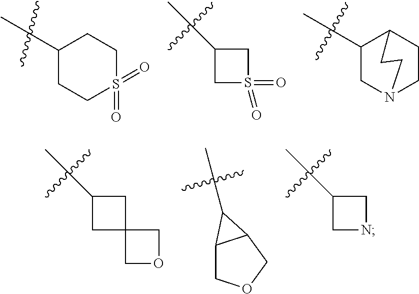

wherein the ring is optionally substituted with 1-3 groups selected from halogen, CN, OH, C₁₋₄ alkyl, hydroxy C₁₋₄ alkyl, and C₁₋₄ haloalkyl;
n is 0, 1, 2, or 3;
m is 0, 1, or 2;
r is 1 or 2; and
t is selected from 0, 1, or 2.

Solution 8 of the present disclosure relates to the compounds as described in solutions 1-6, or stereoisomers or pharmaceutically acceptable salts thereof, wherein each L₂ is independently selected from

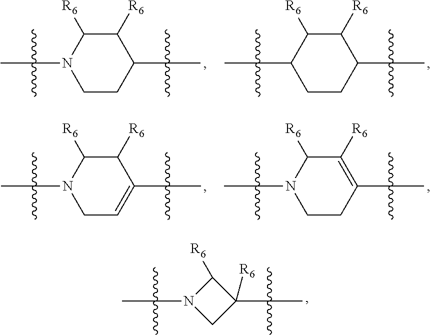

CO, O, NR_{L2}, C₁₋₄ alkylene, C₂₋₄ alkenylene, C₂₋₄ alkynylene,

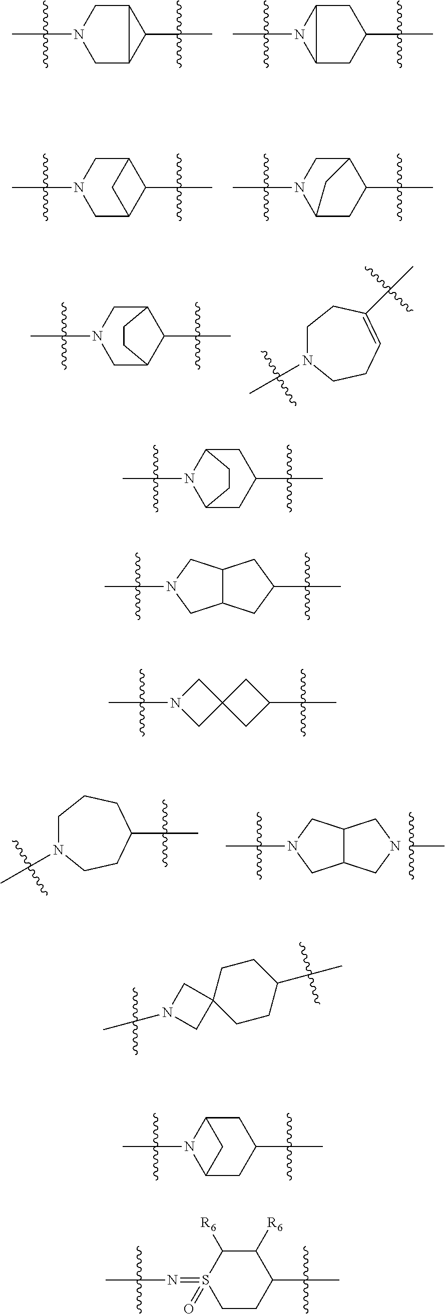

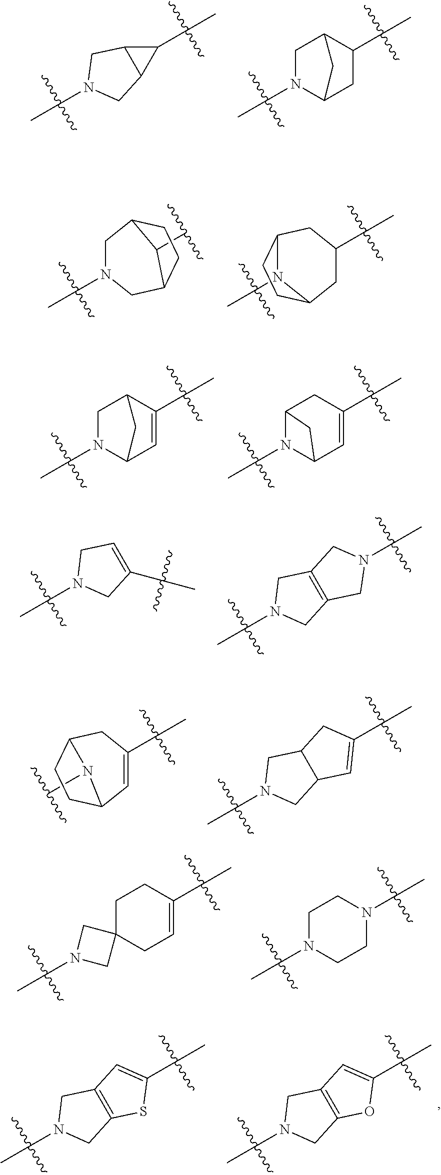

R_{L2} is H, C₁₋₄ alkyl, or C₃₋₆ cycloalkyl;
L₃ is selected from

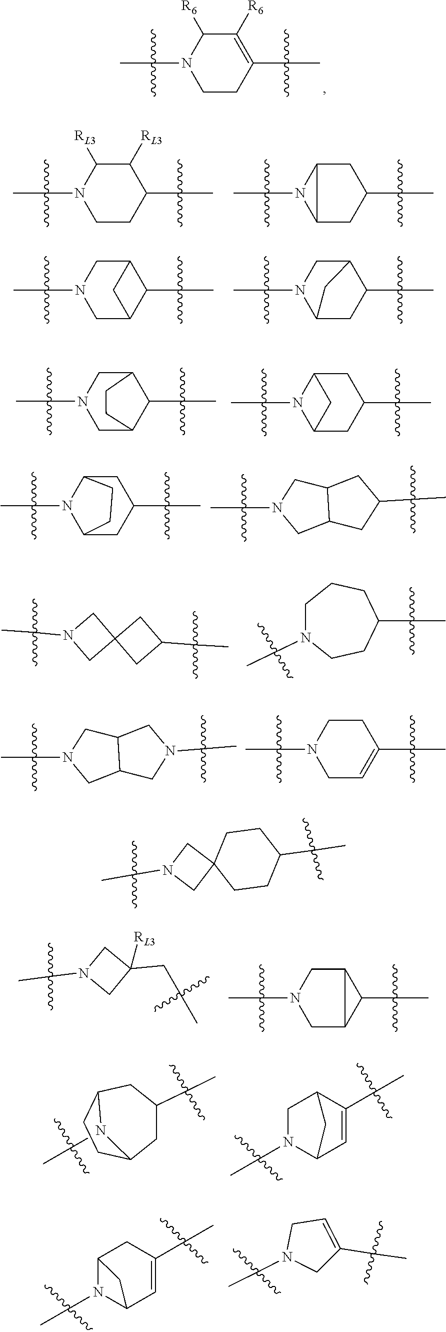

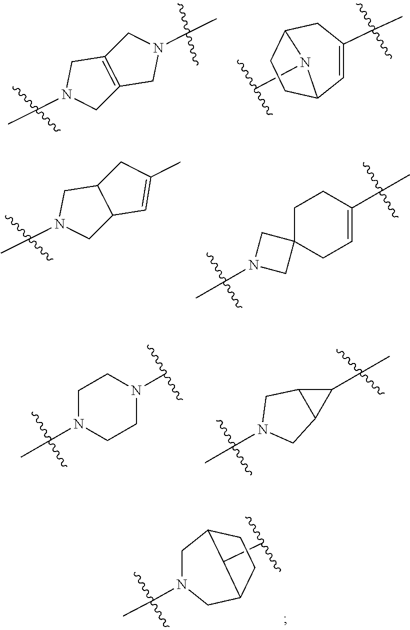

each R_{L3} is independently selected from halogen, ═O, CN, C₁₋₄ alkyl, or C₁₋₄ alkoxy, or R_{L3} and R_{X1}, together with the atom to which they are attached, form C₃₋₆ cycloalkyl or a 5- to 6-membered heterocycloalkyl containing 1-3 heteroatoms selected from N, S, and O;
L₄ is selected from

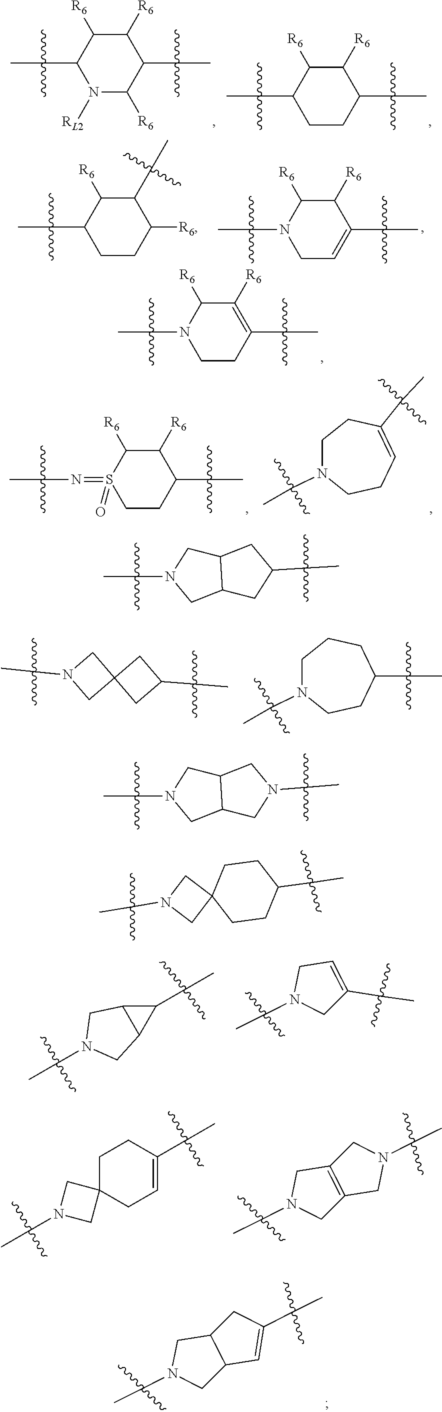

A is —S(O)—, —C(O)—, —B(OH)—, —S—, or —S(═N)—CN;
Cy5 is selected from

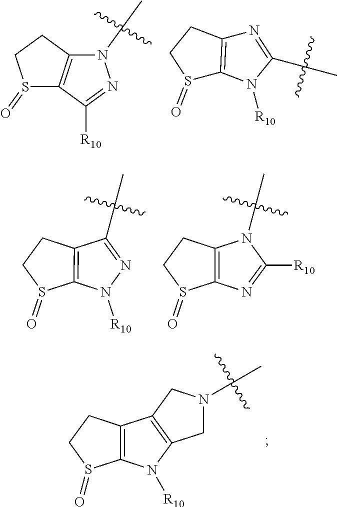

each R_{X1} and R are independently H, halogen, C₁₋₄ alkyl, C₂₋₄ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, or C₁₋₄ haloalkyl, or R_{X1} and R are independently a 3- to 5-membered cycloalkyl, or two R on adjacent ring atoms, R and R_{X1}, or R_{X1} and R₆, together with the atom to which they are attached, form C₃₋₆ cycloalkyl or a 5- to 6-membered heterocycloalkyl containing 1-3 heteroatoms selected from N, S, and O, wherein the cycloalkyl and heterocycloalkyl are optionally substituted with 1-3 groups selected from halogen, ═O, CN, C₁₋₄ alkyl, C₁₋₄ alkoxy, OH, and NH₂;
R₁ is C₁₋₄ alkyl or C₁₋₄ haloalkyl;
or R₁ and R₂, together with the atom to which they are attached, form a ring selected from:

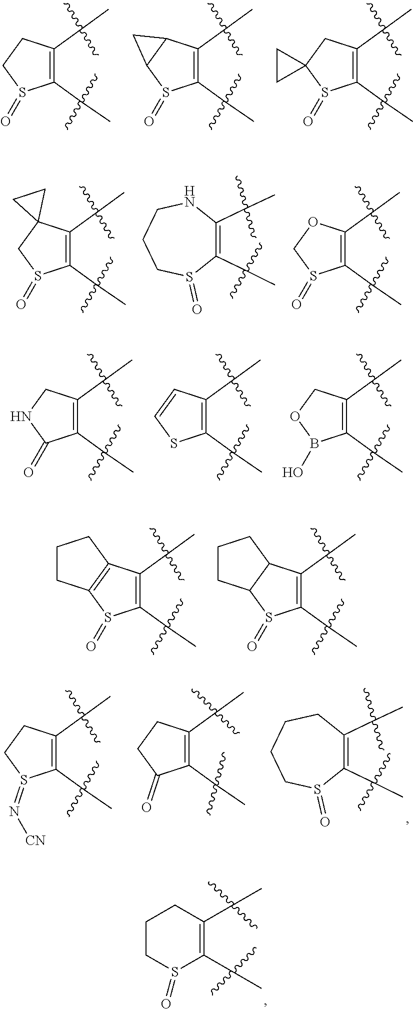

R₁ and R₂, together with the atom to which they are attached, form a ring selected from:

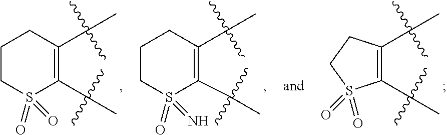 [SMILES: *C1=C(*)S(=O)(=O)CCC1 ; *C1=C(*)S(=N)(=O)CCC1 ; *C1=C(*)S(=O)(=O)CC1]

optionally substituted with 1-3 groups selected from halogen, ═O, CN, C₁₋₄ alkyl, C₁₋₄ alkoxy, OH, and NH₂;
R₃ is H, halogen, C₁₋₄ alkyl, CN, OH, or absent;
or R₃ and R₂, together with the atom to which they are attached, form a ring selected from:

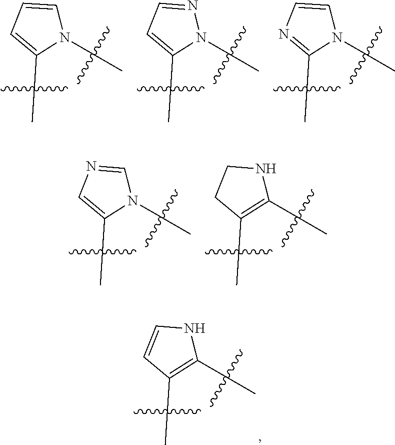 [SMILES: *c1cccn1* ; *c1ccnn1* ; *c1nccn1* ; *c1cncn1* ; *C1=C(*)NCC1 ; *c1cc[nH]c1*]

optionally substituted with 1-3 groups selected from halogen, ═O, CN, C₁₋₄ alkyl, C₁₋₄ alkoxy, OH, and NH₂;
or R₃ and R₄ or R₃ and R₅, together with the atom to which they are attached, form a 5- to 6-membered heterocycloalkyl or a 5- to 6-membered heteroaryl containing 1-3 heteroatoms selected from N, S, and O, or phenyl, wherein the heterocycloalkyl, phenyl, and heteroaryl are optionally substituted with 1-3 groups selected from halogen, ═O, CN, C₁₋₄ alkyl, C₁₋₄ alkoxy, OH, and NH₂;
or R₃ and R₆, together with the atom to which they are attached, form C₅₋₆ cycloalkyl, or a 5- to 6-membered heterocycloalkyl containing 1-3 heteroatoms selected from N, S, and O, optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent; or Rₓ₄ and R₄ or Rₓ₄ and R₅, together with the atom to which they are attached, form imidazolyl, pyrazolyl, pyrrolyl, thienyl, or thiazolyl, optionally substituted with 1-3 groups selected from halogen, ═O, CN, C₁₋₄ alkyl, C₁₋₄ alkoxy, OH, and NH₂;
R₄ is H, C₁₋₄ alkyl, or C₁₋₄ haloalkyl;
R₅ is H, C₁₋₄ alkyl, C₁₋₄ haloalkyl, C₁₋₄ alkoxy, or —NHC₁₋₄ alkyl;
R₆ is H, halogen, ═O, CN, C₁₋₄ alkyl, or C₁₋₄ alkoxy;
R₇ is C₁₋₄ alkyl, or C₁₋₄ haloalkyl, or R₇ and R₉, together with the atom to which they are attached, form a ring selected from:

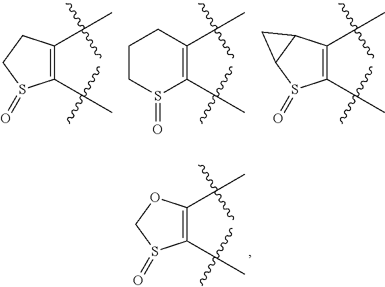

wherein the ring is optionally substituted with 1-3 groups selected from halogen, CN, C₁₋₄ alkyl, C₁₋₄ alkoxy, OH, and NH₂;
or R_{9′} and R₇, together with the atom to which they are attached, form a ring selected from:

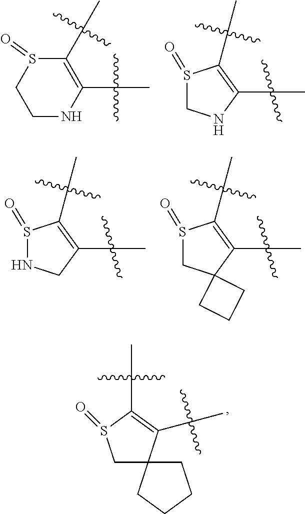 [SMILES: *C1=C(*)S(=O)CCN1 ; *C1=C(*)S(=O)CN1 ; *C1=C(*)S(=O)NC1 ; *C1=C(*)C2(CCC2)CS1=O ; *C1=C(*)C2(CCCC2)CS1=O]

optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
or R_{9′} and Rₓ₅, together with the atom to which they are attached, form

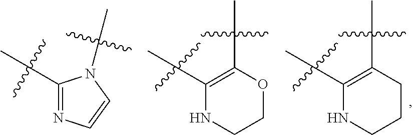 [SMILES: *c1nccn1* ; *C1=C(*)OCCN1 ; *C1=C(*)NCCC1]

optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
provided that at least one group of R_{9′} and R₇ or R_{9′} and Rₓ₅ form a ring;
R₈ is R_{8′}, —OR_{8′}, or —(CH₂)ᵣR_{8′};
R_{8′} is C₃₋₆ cycloalkyl, a 4- to 8-membered heterocycloalkyl containing 1-3 heteroatoms selected from N, S, O, Si, and P, a 5- to 6-membered heteroaryl containing 1-3 heteroatoms selected from N, S, and O, or —N═S(═O)(C₁₋₄ alkyl)₂, or R_{8′} is —N(C₃₋₆ cycloalkyl)C(O)NH(C₁₋₄ alkyl), wherein the cycloalkyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, and CN;
R₉ is H or C₁₋₄ alkyl;
each R₁₀ is independently R_{8′}, —OR_{8′}, —NHR_{8′}, or —(CH₂)ᵣR_{8′};
R₁₁ is C₃₋₆ cycloalkyl optionally substituted with 1-3 groups selected from halogen, CN, ═O, OH, C₁₋₄ alkyl, hydroxy C₁₋₄ alkyl, and C₁₋₄ haloalkyl;
R₁₂ is selected from cyclobutyl, cyclopentyl, cyclohexyl, phenyl, cyclopropyl, adamantyl, tetrahydropyranyl, piperidinyl, oxolanyl, oxanyl,

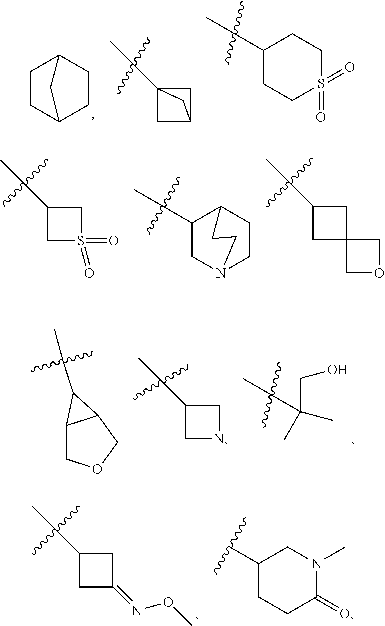

C₁₋₄ alkyl-CN, —C(NHC₁₋₂ alkyl)=N—CN, —C(C₁₋₂ alkyl)=N—CN, —C(NHC₁₋₂ alkyl)=CH—NO₂, —CH(CH₃)—CH₂—NH—C(O)O(CH₃), —C(CH₃)₂—CH₂—NH—C(O)O(CH₃), —CH(CH₃)—CH₂—O—C(O)NH₂, —C(CH₃)₂—CH₂—O—C(O)NH₂, —C(O)—Ra, —CH(CH₃)—CH₂—O—C(O)NHCH₃, or —C(CH₃)₂—CH₂—O—C(O)NHCH₃; wherein the ring is optionally substituted with 1-3 groups selected from halogen, CN, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NH—C(O)NH₂, —(CH₂)ₜ—NH—C(═NH)NH₂, —(CH₂)ₜ—NH—C(O)—O—(C₁₋₂ alkyl), —(CH₂)ₜ—NH—C(C₁₋₂ alkyl)=N—CN, —(CH₂)ₜ—NH—C(NHC₁₋₂ alkyl)=N—CN, —(CH₂)ₜ—NH—C(NHC₁₋₂ alkyl)=CH—NO₂, —(CH₂)ₜ—C(NHC₁₋₂ alkyl)=N—CN, —(CH₂)ₜ—C(C₁₋₂ alkyl)=N—CN, —(CH₂)ₜ—C(C₁₋₂ alkyl)=N—O—(C₁₋₂ alkyl), ═N—O—C₁₋₂ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃;
or R₁₂ is selected from cyclobutyl, cyclopentyl, cyclohexyl, phenyl, cyclopropyl, adamantyl, tetrahydropyranyl, piperidinyl, oxolanyl, oxanyl,

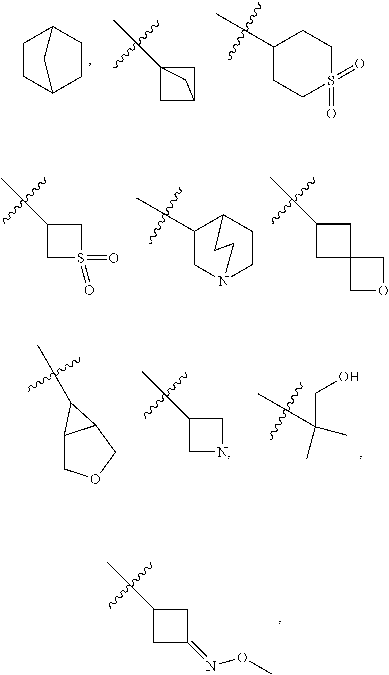

—C(NHC₁₋₂ alkyl)=N—CN, —C(C₁₋₂ alkyl)=N—CN, or —C(NHC₁₋₂ alkyl)=CH—NO₂, or from —C(CH₃)ₜ—CH₂—O—C(O)NH₂ and —C(O)—Ra, or is —C(CH₃)ₜ—CH₂—O—C(O)NHCH₃; the ring is optionally substituted with 1-3 groups selected from halogen, CN, OH, —SF₅, C₁₋₄ alkyl, hydroxy C₁₋₄ alkyl, C₁₋₄ haloalkyl, —(CH₂)ₜ—O—C(O)NH₂, —(CH₂)ₜ—NH—C(O)NH₂, —(CH₂)ₜ—NH—C(═NH)NH₂, —(CH₂)ₜ—NH—C(O)—O—(C₁₋₂ alkyl), —(CH₂)ₜ—NH—C(C₁₋₂ alkyl)=N—CN, —(CH₂)ₜ—NH—C(NHC₁₋₂ alkyl)=N—CN, —(CH₂)ₜ—NH—C(NHC₁₋₂ alkyl)=CH—NO₂, —(CH₂)ₜ—C(NHC₁₋₂ alkyl)=N—CN, —(CH₂)ₜ—C(C₁₋₂ alkyl)=N—CN, —(CH₂)ₜ—C(C₁₋₂ alkyl)=N—O—(C₁₋₂ alkyl), ═N—O—C₁₋₂ alkyl, and a 5- to 6-membered heteroaryl, or substituted with —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, or —(CH₂)ₜ—NRa—C(O)CH₃;
R₁₃ is selected from

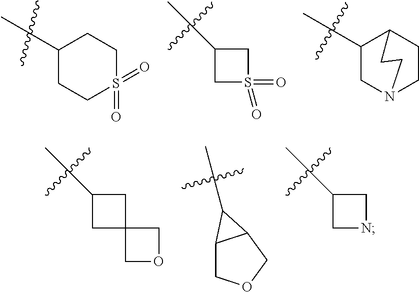

wherein the ring is optionally substituted with 1-3 groups selected from halogen, CN, OH, C₁₋₄ alkyl, hydroxy C₁₋₄ alkyl, and C₁₋₄ haloalkyl;
n is 0, 1, 2, or 3;
m is 0, 1, or 2;
r is 1 or 2; and
t is selected from 0, 1, or 2.

Solution 9 of the present disclosure relates to the compounds as described in solutions 1-6, or stereoisomers or pharmaceutically acceptable salts thereof, wherein each L₂ is independently selected from

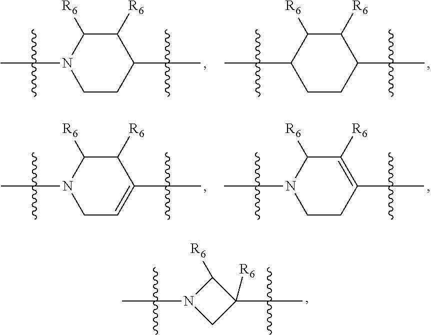

CO, O, NR_{L2}, C₁₋₄ alkylene, C₂₋₄ alkenylene, C₂₋₄ alkynylene,

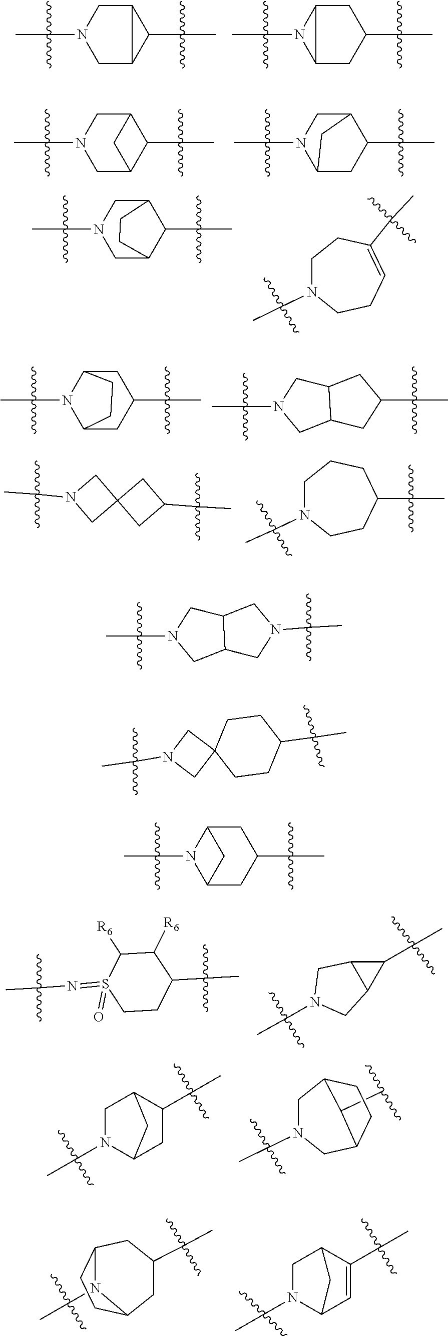

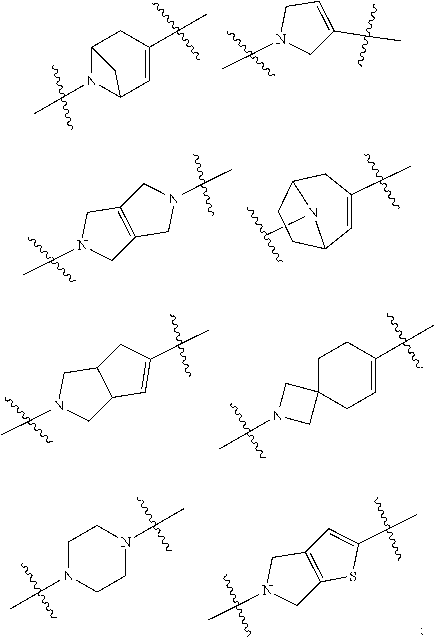

or
each L₂ is independently selected from

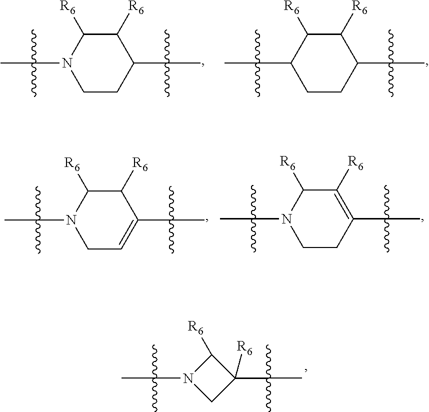

CO, O, NR_{L2}, C₁₋₄ alkylene, C₂₋₄ alkenylene, C₂₋₄ alkynylene,

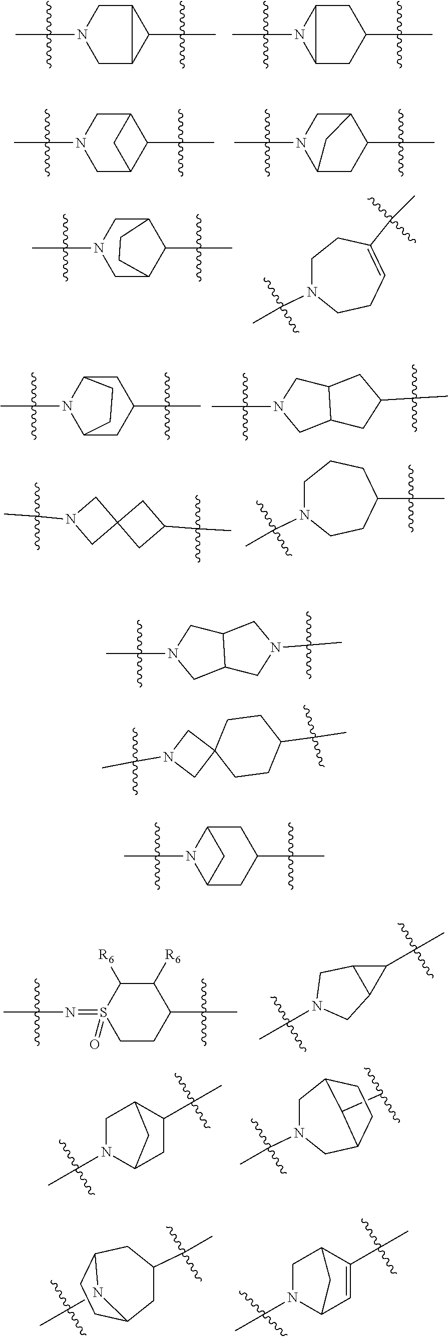

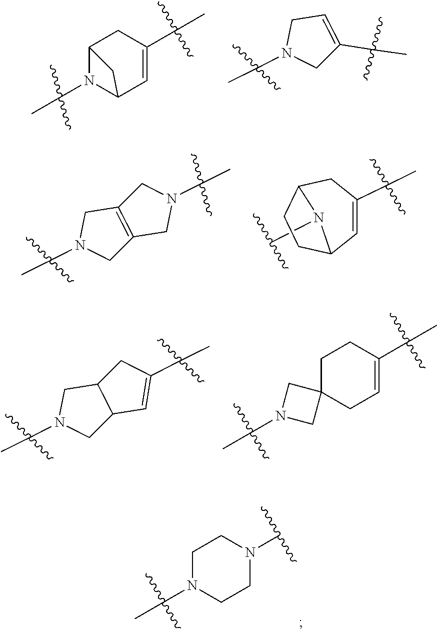

or each L₂ is independently selected from

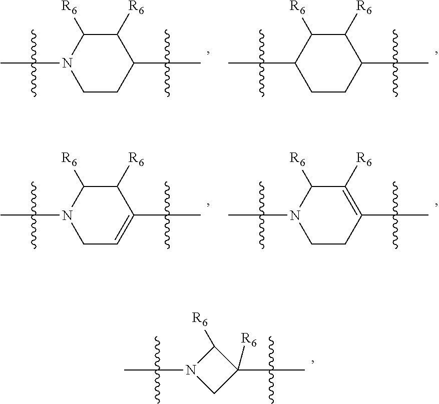

CO, O, NR_{L2}, C₁₋₄ alkylene, C₂₋₄ alkenylene, C₂₋₄ alkynylene,

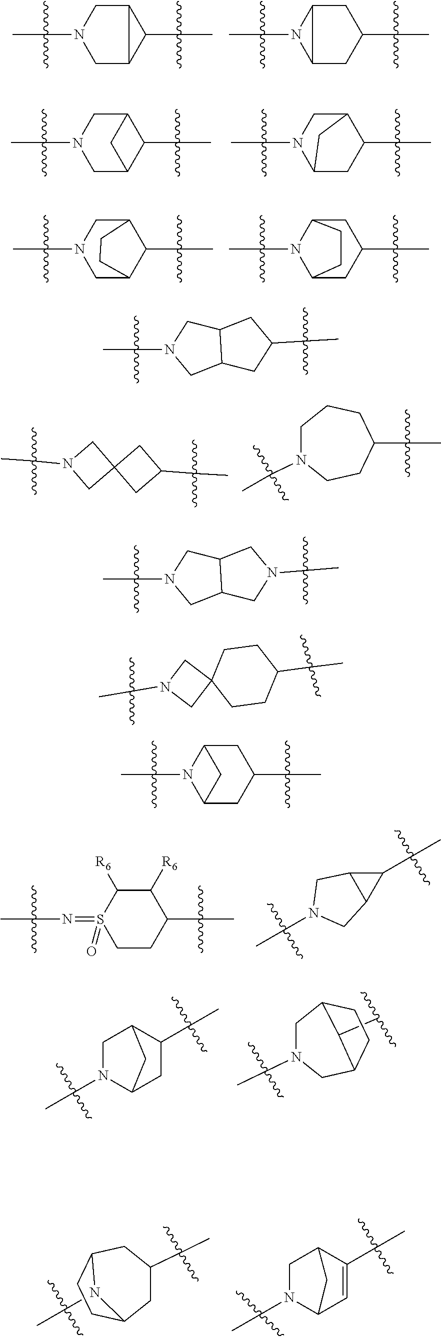

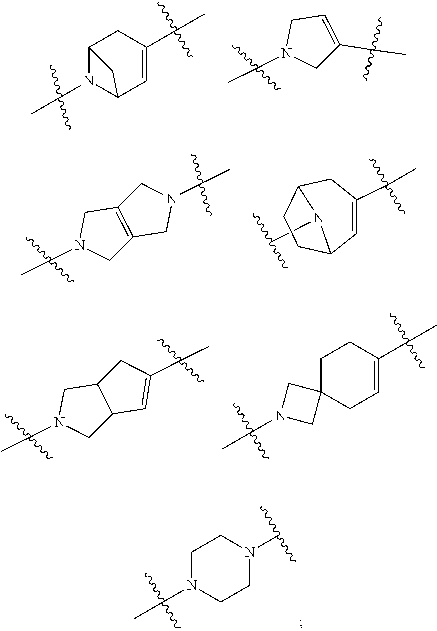

or each L₂ is independently selected from

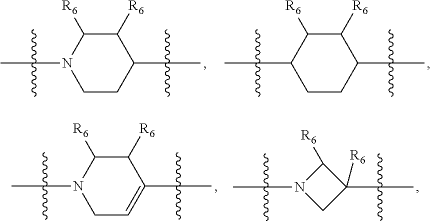

CO, O, NR_{L2}, C₁₋₄ alkylene, C₂₋₄ alkenylene, C₂₋₄ alkynylene,

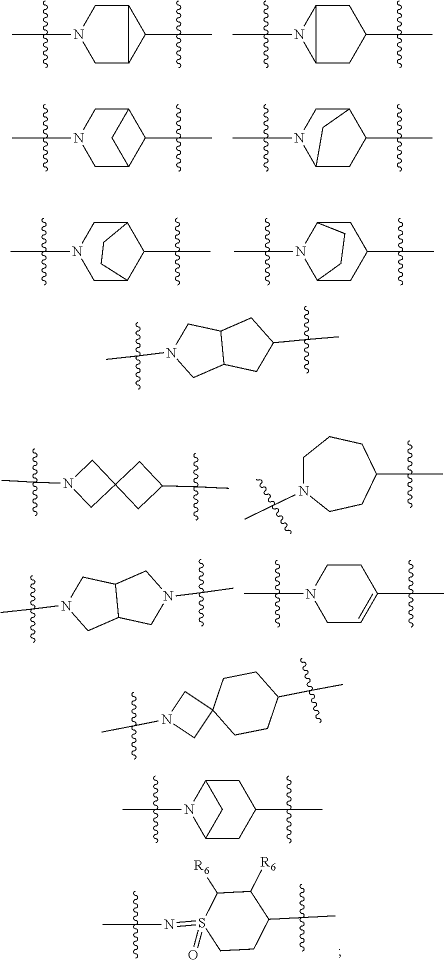

R_{L2} is H, C₁₋₄ alkyl, or C₃₋₆ cycloalkyl;
L₃ is selected from

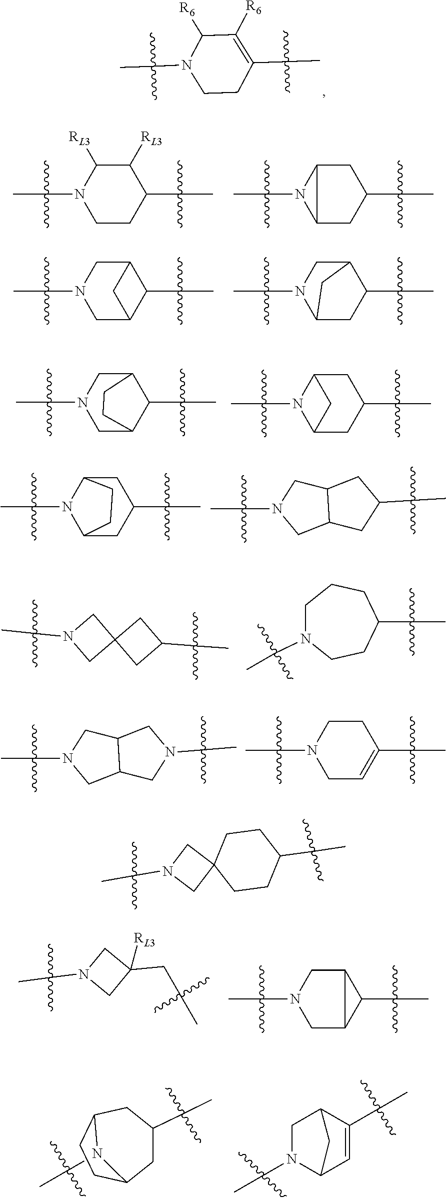

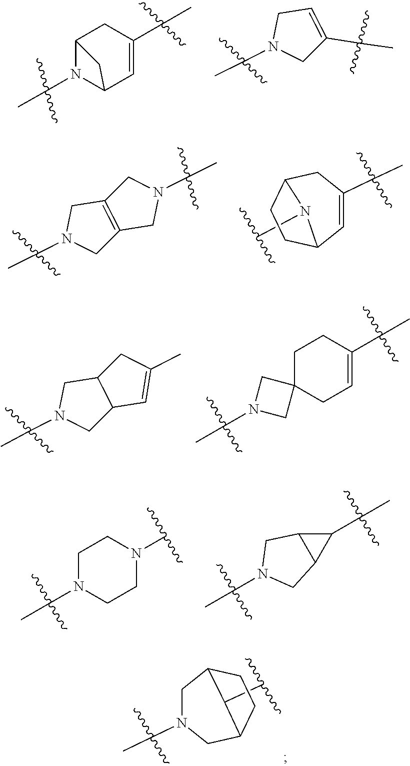

or L₃ is selected from

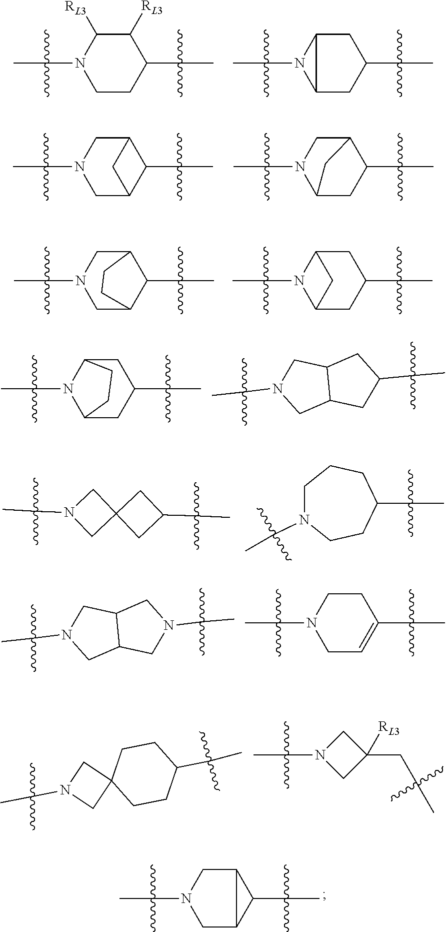

each R_{L3} is independently selected from halogen, ═O, CN, C₁₋₄ alkyl, or C₁₋₄ alkoxy, or R_{L3} and R_{X1}, together with the atom to which they are attached, form a C₃₋₆ cycloalkyl or a 5- to 6-membered heterocycloalkyl containing 1-3 heteroatoms selected from N, S, and O;
A is —S(O)—, —C(O)—, —B(OH)—, —S—, or —S(═N)—CN;
Cy5 is selected from

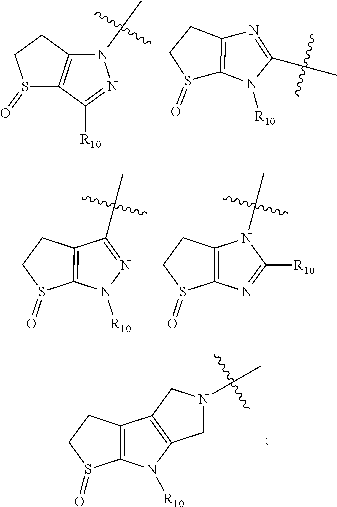

each R_{X1} and R are independently H, halogen, C₁₋₄ alkyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, or C₁₋₄ haloalkyl, or R_{X1} and R are independently 3- to 5-membered cycloalkyl, or two R on adjacent ring atoms, R and R_{X1}, or R_{X1} and R₆, together with the atom to which they are attached, form C₃₋₆ cycloalkyl, wherein the cycloalkyl is optionally substituted with 1-3 groups selected from halogen, ═O, CN, C₁₋₄ alkyl, C₁₋₄ alkoxy, OH, and NH₂;
R₁ is C₁₋₄ alkyl or C₁₋₄ haloalkyl;
or R₁ and R₂, together with the atom to which they are attached, form a ring selected from:

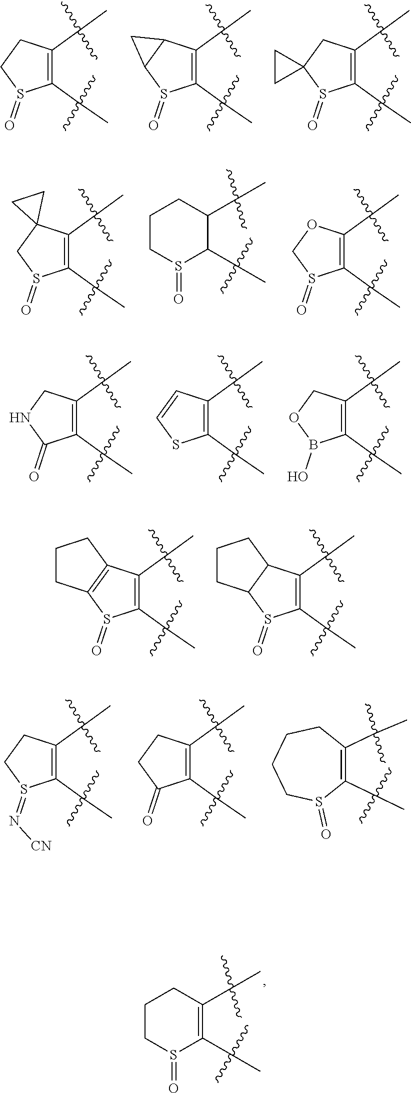

optionally substituted with 1-3 groups selected from halogen, ═O, CN, C₁₋₄ alkyl, C₁₋₄ alkoxy, OH, and NH₂; or R₁ and R₂, together with the atom to which they are attached, form a ring selected from:

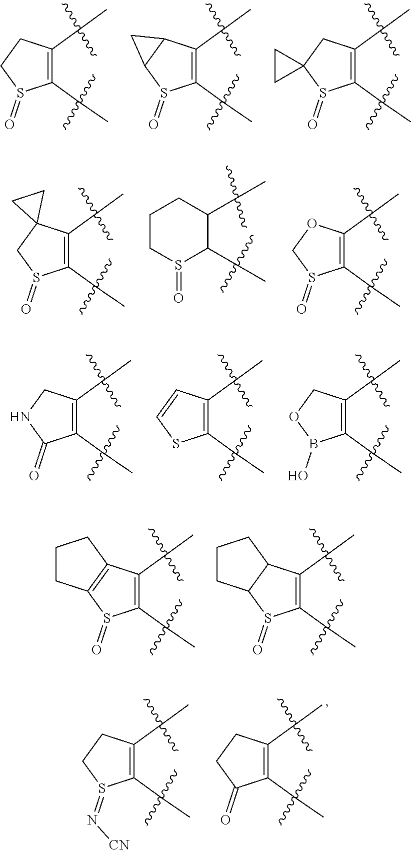

optionally substituted with 1-3 groups selected from halogen, ═O, CN, C₁₋₄ alkyl, C₁₋₄ alkoxy, OH, and NH₂;
R₃ is H, halogen, C₁₋₄ alkyl, CN, OH, or absent;
or R₃ and R₂, together with the atom to which they are attached, form a ring selected from:

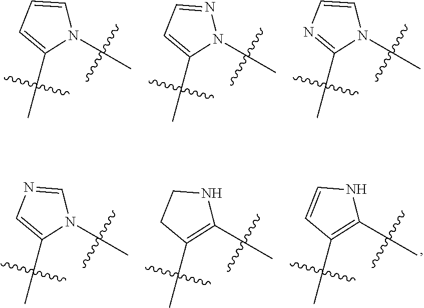 [SMILES: *c1cccn1* ; *c1ccnn1* ; *c1nccn1* ; *c1cncn1* ; *C1=C(*)NCC1 ; *c1cc[nH]c1*]

optionally substituted with 1-3 groups selected from halogen, ═O, CN, C₁₋₄ alkyl, C₁₋₄ alkoxy, OH, and NH₂;
or R₃ and R₄ or R₃ and R₅, together with the atom to which they are attached, form a 5- to 6-membered heterocycloalkyl or a 5- to 6-membered heteroaryl containing 1-3 heteroatoms selected from N, S, and O, or phenyl, wherein the heterocycloalkyl, phenyl, and heteroaryl are optionally substituted with 1-3 groups selected from halogen, ═O, CN, C₁₋₄ alkyl, C₁₋₄ alkoxy, OH, and NH₂;
or R₃ and R₆, together with the atom to which they are attached, form C₅₋₆ cycloalkyl, or a 5- to 6-membered heterocycloalkyl containing 1-3 heteroatoms selected from N, S, and O, optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent; or Rₓ₄ and R₄ or Rₓ₄ and R₅, together with the atom to which they are attached, form imidazolyl, pyrazolyl, pyrrolyl, thienyl, or thiazolyl, optionally substituted with 1-3 groups selected from halogen, ═O, CN, C₁₋₄ alkyl, C₁₋₄ alkoxy, OH, and NH₂;
R₄ is H, C₁₋₄ alkyl, or C₁₋₄ haloalkyl;
R₅ is H, C₁₋₄ alkyl, C₁₋₄ haloalkyl, C₁₋₄ alkoxy, or —NHC₁₋₄ alkyl;
R₆ is H, halogen, ═O, CN, C₁₋₄ alkyl, or C₁₋₄ alkoxy;
R₇ is C₁₋₄ alkyl, or C₁₋₄ haloalkyl, or R₇ and R₉, together with the atom to which they are attached, form a ring selected from:

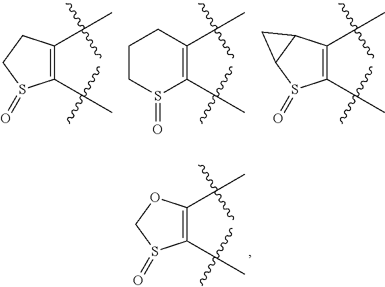

wherein the ring is optionally substituted with 1-3 groups selected from halogen, CN, C₁₋₄ alkyl, C₁₋₄ alkoxy, OH, and NH₂;
or R_{9′} and R₇, together with the atom to which they are attached, form a ring selected from:

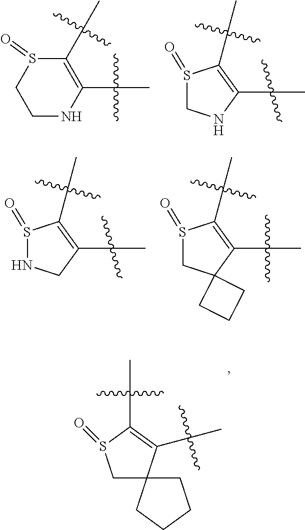 [SMILES: *C1=C(*)S(=O)CCN1 ; *C1=C(*)S(=O)CN1 ; *C1=C(*)S(=O)NC1 ; *C1=C(*)C2(CCC2)CS1=O ; *C1=C(*)C2(CCCC2)CS1=O]

optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
or R_{9′} and Rₓ₅, together with the atom to which they are attached, form

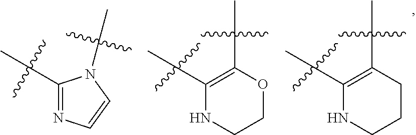 [SMILES: *c1nccn1* ; *C1=C(*)OCCN1 ; *C1=C(*)NCCC1]

optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
provided that at least one group of R_{9′} and R₇ or R_{9′} and Rₓ₅ form a ring;
R₈ is R_{8′}, —OR_{8′}, or —(CH₂)ᵣR_{8′};
R_{8′} is C₃₋₆ cycloalkyl, wherein the cycloalkyl is optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, and CN;
R₉ is H or C₁₋₄ alkyl;
each R₁₀ is independently R_{8′}, —OR_{8′}, —NHR_{8′}, or —(CH₂)ᵣR_{8′};
R₁₁ is C₃₋₆ cycloalkyl optionally substituted with 1-3 groups selected from halogen, CN, ═O, OH, C₁₋₄ alkyl, hydroxy C₁₋₄ alkyl, and C₁₋₄ haloalkyl;
n is 0, 1, 2, or 3;
m is 0, 1, or 2; and
r is 1 or 2.

Solution 10 of the present disclosure relates to the compounds or the stereoisomers or pharmaceutically acceptable salts thereof as described above in solutions 1-6, wherein
each L₂ is independently selected from

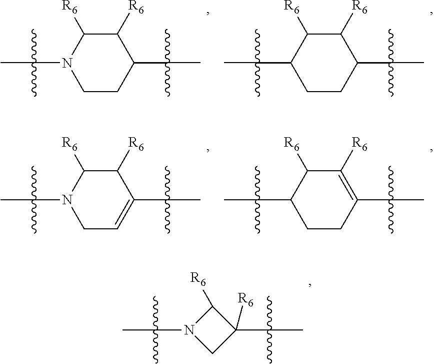

CO, O, NR_{L2}, C₁₋₄ alkylene, C₂₋₄ alkenylene, C₂₋₄ alkynylene,

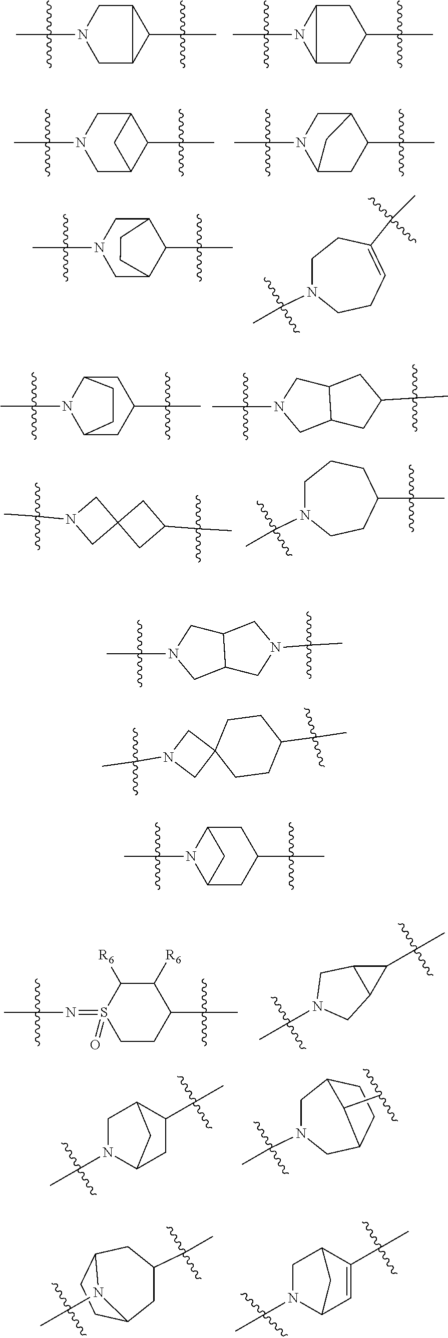

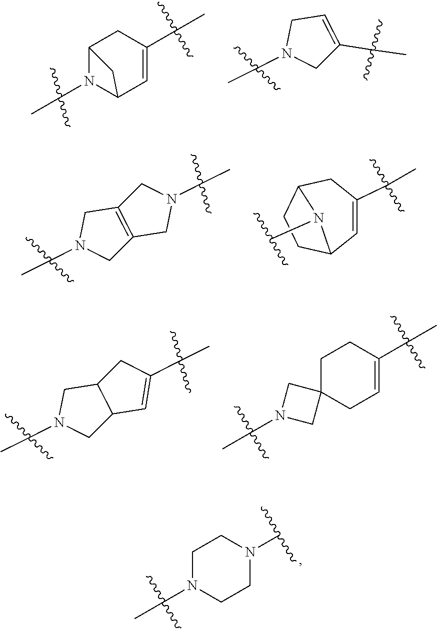

or each L₂ is independently selected from

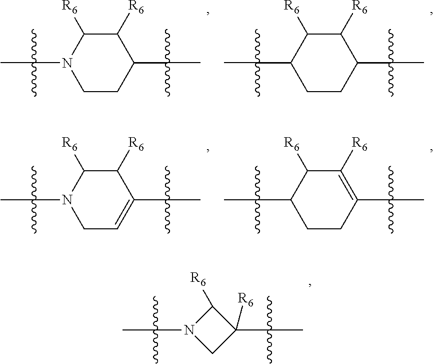

CO, O, NR_{L2}, C₁₋₄ alkylene, C₂₋₄ alkenylene, C₂₋₄ alkynylene,

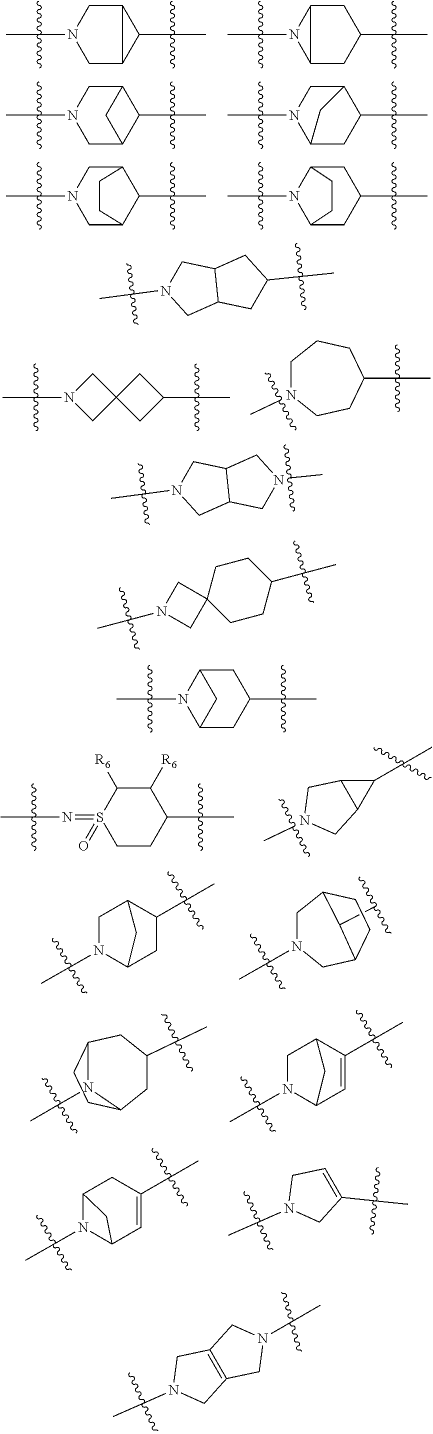

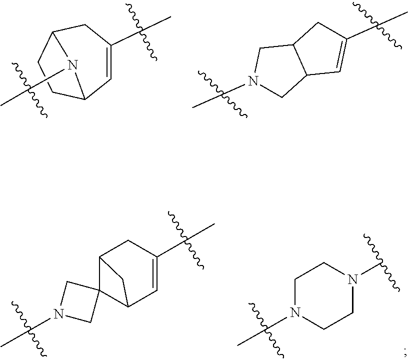 [SMILES: *C1=CC2CCC(C1)N2* ; *C1=CC2CN(*)CC2C1 ; *C1=CC2CC(C1)C21CN(*)C1 ; *N1CCN(*)CC1]

R_{L2} is H, C₁₋₄ alkyl, or C₃₋₆ cycloalkyl;
L₃ is selected from

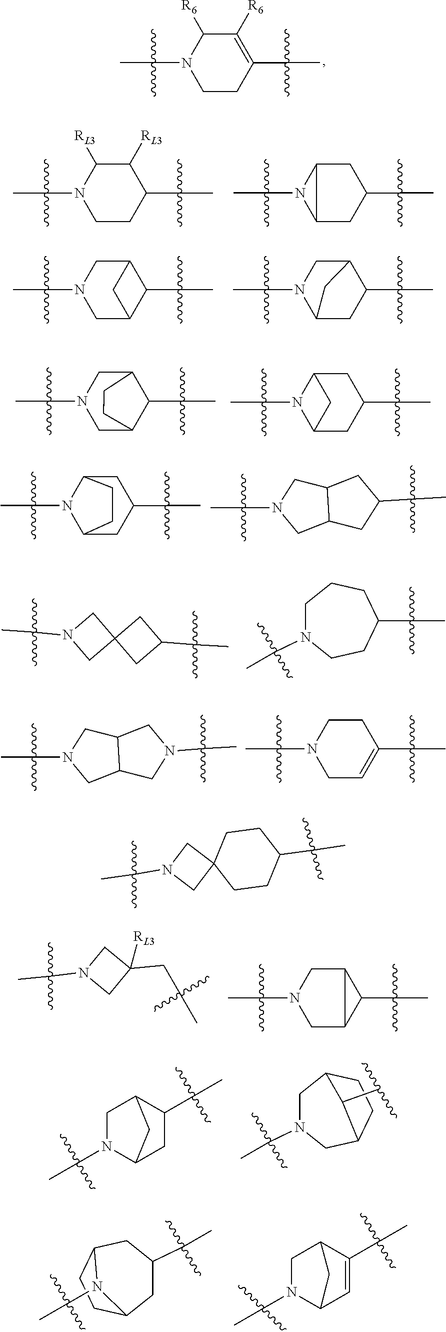

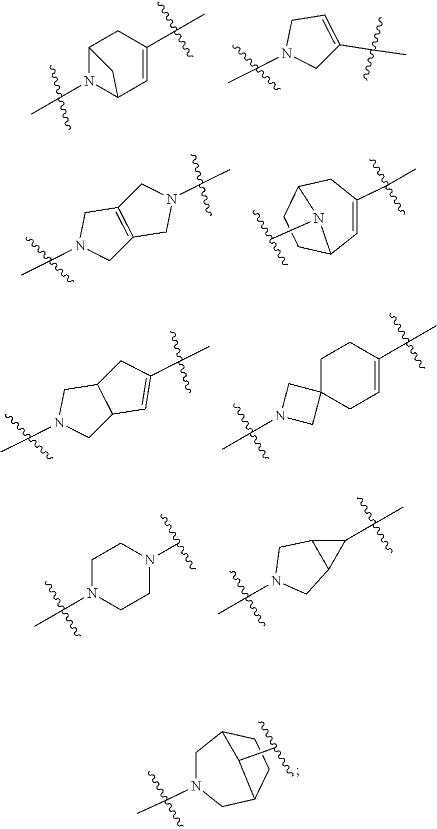

each R_{L3} is independently selected from halogen, ═O, CN, C₁₋₄ alkyl, or C₁₋₄ alkoxy, or R_{L3} and R_{X1}, together with the atom to which they are attached, form C₃₋₆ cycloalkyl or a 5- to 6-membered heterocycloalkyl containing 1-3 heteroatoms selected from N, S, and O;
L₄ is selected from

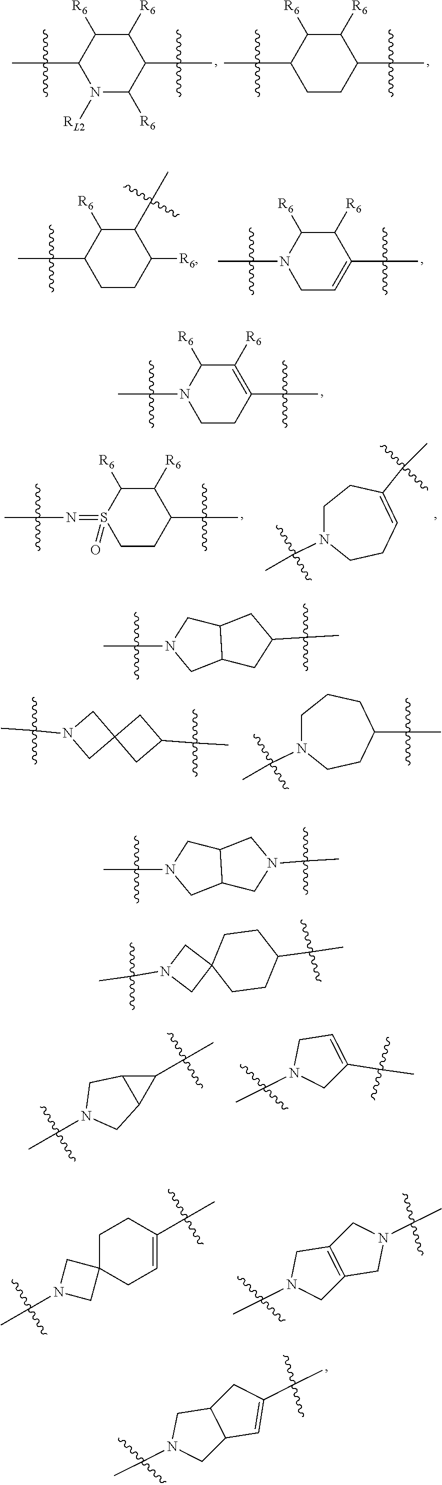

or L₄ is selected from

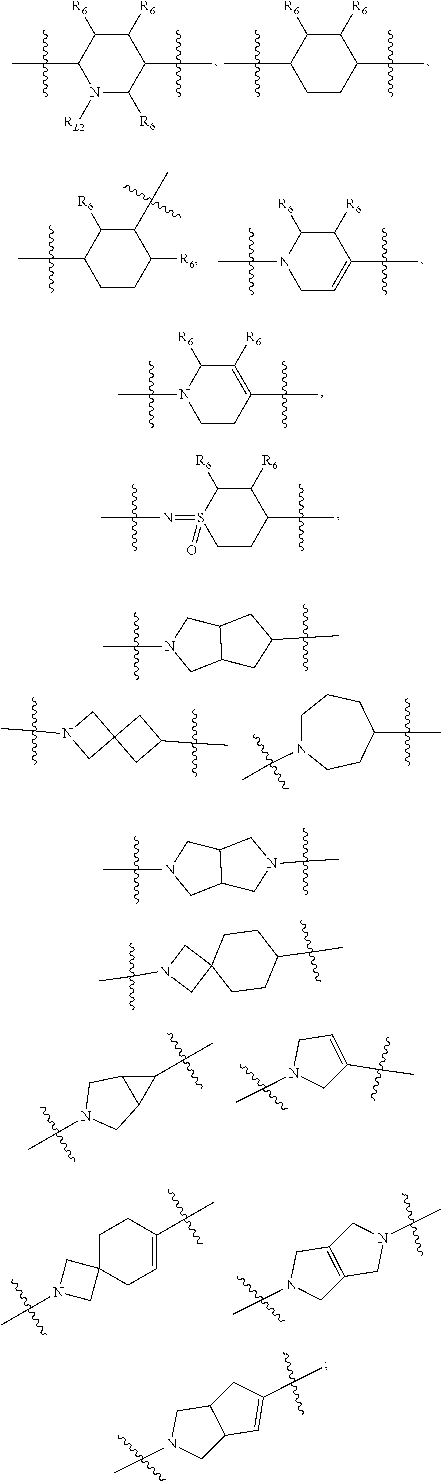

A is —S(O)—, —C(O)—, —B(OH)—, —S—, or —S(═N)—CN;
Cy5 is selected from

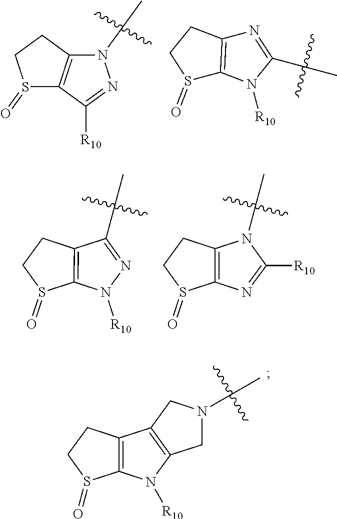

each R_{X1} and R are independently H, halogen, C₁₋₄ alkyl, C₂₋₄ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, or C₁₋₄ haloalkyl, or R_{X1} and R are independently a 3- to 5-membered cycloalkyl, or two R on adjacent ring atoms, R and R_{X1}, or R_{X1} and R₆, together with the atom to which they are attached, form C₃₋₆ cycloalkyl or a 5- to 6-membered heterocycloalkyl containing 1-3 heteroatoms selected from N, S, and O, wherein the cycloalkyl and heterocycloalkyl are optionally substituted with 1-3 groups selected from halogen, ═O, CN, C₁₋₄ alkyl, C₁₋₄ alkoxy, OH, and NH₂;
R₁ is C₁₋₄ alkyl or C₁₋₄ haloalkyl;
or R₁ and R₂, together with the atom to which they are attached, form a ring selected from:

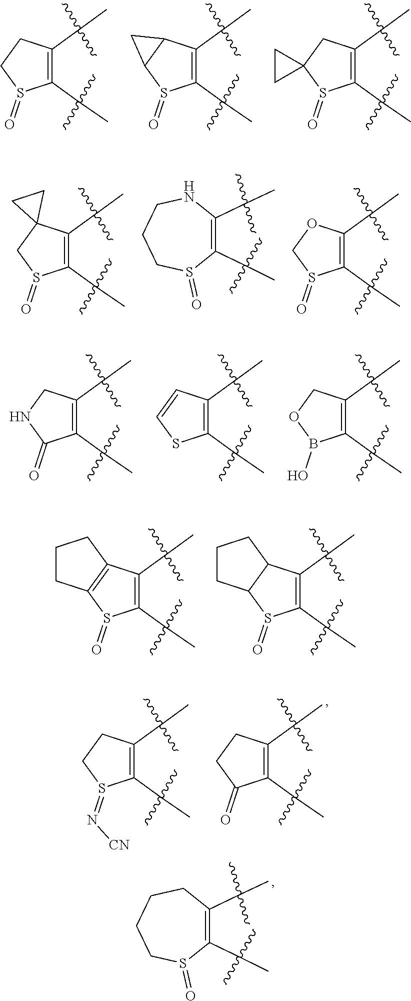

or R₁ and R₂, together with the atom to which they are attached, form

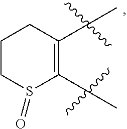 [SMILES: *C1=C(*)S(=O)CCC1]

optionally substituted with 1-3 groups selected from halogen, ═O, CN, C₁₋₄ alkyl, C₁₋₄ alkoxy, OH, and NH₂;
R₃ is H, halogen, C₁₋₄ alkyl, CN, OH, or absent;
or R₃ and R₂, together with the atom to which they are attached, form a ring selected from:

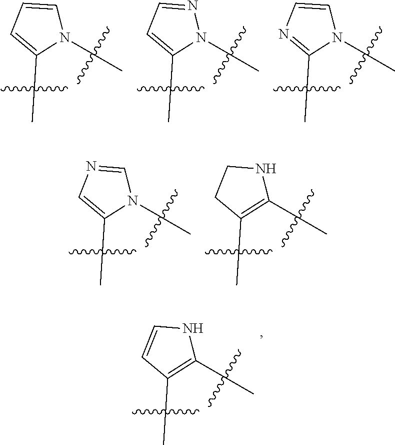 [SMILES: *c1cccn1* ; *c1ccnn1* ; *c1nccn1* ; *c1cncn1* ; *C1=C(*)NCC1 ; *c1cc[nH]c1*]

optionally substituted with 1-3 groups selected from halogen, ═O, CN, C₁₋₄ alkyl, C₁₋₄ alkoxy, OH, and NH₂;
or R₃ and R₄ or R₃ and R₅, together with the atom to which they are attached, form a 5- to 6-membered heterocycloalkyl or a 5- to 6-membered heteroaryl containing 1-3 heteroatoms selected from N, S, and O, or phenyl, wherein the heterocycloalkyl, phenyl, and heteroaryl are optionally substituted with 1-3 groups selected from halogen, ═O, CN, C₁₋₄ alkyl, C₁₋₄ alkoxy, OH, and NH₂;
or R₃ and R₆, together with the atom to which they are attached, form C₅₋₆ cycloalkyl, or a 5- to 6-membered heterocycloalkyl containing 1-3 heteroatoms selected from N, S, and O, optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent; or Rₓ₄ and R₄ or Rₓ₄ and R₅, together with the atom to which they are attached, form imidazolyl, pyrazolyl, pyrrolyl, thienyl, or thiazolyl, optionally substituted with 1-3 groups selected from halogen, ═O, CN, C₁₋₄ alkyl, C₁₋₄ alkoxy, OH, and NH₂;
R₄ is H, C₁₋₄ alkyl, or C₁₋₄ haloalkyl;
R₅ is H, C₁₋₄ alkyl, C₁₋₄ haloalkyl, C₁₋₄ alkoxy, or —NHC₁₋₄ alkyl;
R₆ is H, halogen, ═O, CN, C₁₋₄ alkyl, or C₁₋₄ alkoxy;
R₇ is C₁₋₄ alkyl, or C₁₋₄ haloalkyl, or R₇ and R₉, together with the atom to which they are attached, form a ring selected from:

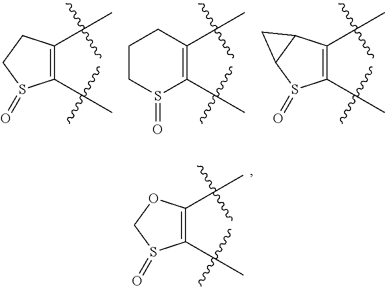

wherein the ring is optionally substituted with 1-3 groups selected from halogen, CN, C₁₋₄ alkyl, C₁₋₄ alkoxy, OH, and NH₂;
or R_{9′} and R₇, together with the atom to which they are attached, form a ring selected from:

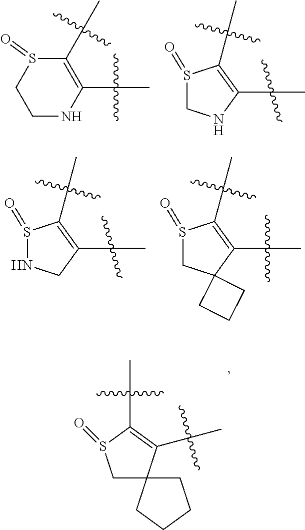 [SMILES: *C1=C(*)S(=O)CCN1 ; *C1=C(*)S(=O)CN1 ; *C1=C(*)S(=O)NC1 ; *C1=C(*)C2(CCC2)CS1=O ; *C1=C(*)C2(CCCC2)CS1=O]

optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
or R_{9′} and Rₓ₅, together with the atom to which they are attached, form

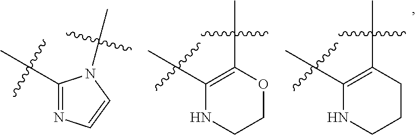 [SMILES: *c1nccn1* ; *C1=C(*)OCCN1 ; *C1=C(*)NCCC1]

optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
provided that at least one group of R_{9′} and R₇ or R_{9′} and Rₓ₅ form a ring;
R₈ is R_{8′}, —OR_{8′}, or —(CH₂)ᵣR_{8′};
R_{8′} is C₃₋₆ cycloalkyl, wherein the cycloalkyl is optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, and CN;
R₉ is H or C₁₋₄ alkyl;
each R₁₀ is independently R_{8′}, —OR_{8′}, —NHR_{8′}, or —(CH₂)ᵣR_{8′};
R₁₁ is C₃₋₆ cycloalkyl optionally substituted with 1-3 groups selected from halogen, CN, ═O, OH, C₁₋₄ alkyl, hydroxy C₁₋₄ alkyl, and C₁₋₄ haloalkyl;
R₁₂ is selected from cycloalkyl, cyclohexyl, phenyl, cyclopropyl, adamantyl, tetrahydropyranyl, piperidinyl, oxolanyl,

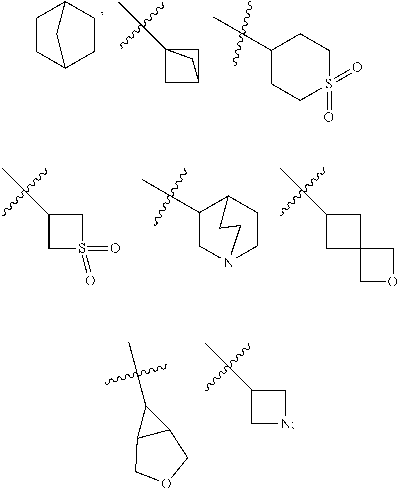

wherein the ring is optionally substituted with 1-3 groups selected from halogen, CN, OH, C₁₋₄ alkyl, hydroxy C₁₋₄ alkyl, and C₁₋₄ haloalkyl;
R₁₃ is selected from

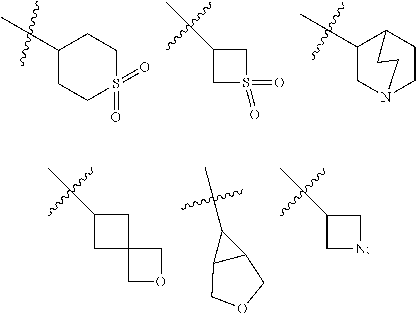

wherein the ring is optionally substituted with 1-3 groups selected from halogen, CN, OH, C₁₋₄ alkyl, hydroxy C₁₋₄ alkyl, and C₁₋₄ haloalkyl;
n is 0, 1, 2, or 3;
m is 0, 1, or 2; and
r is 1 or 2.

Solution 11 of the present disclosure relates to the compounds, or the stereoisomers or pharmaceutically acceptable salts thereof, as described above in solutions 1-7, wherein
Cy1 is selected from

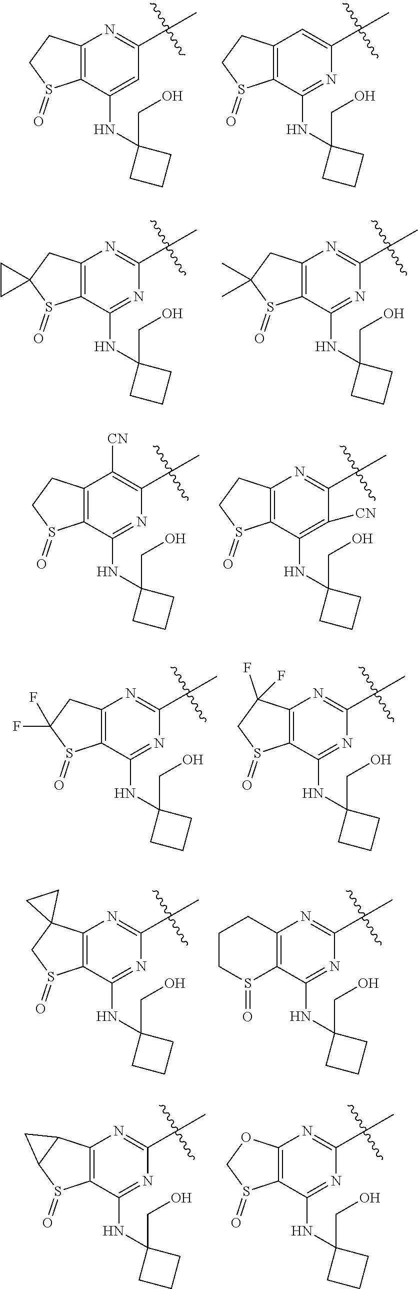

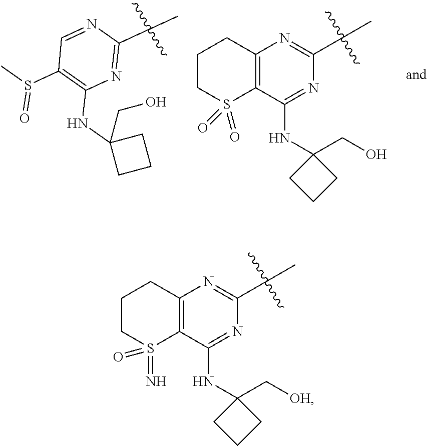 [SMILES: *c1ncc(S(C)=O)c(NC2(CO)CCC2)n1 ; *c1nc2c(c(NC3(CO)CCC3)n1)S(=O)(=O)CCC2 ; *c1nc2c(c(NC3(CO)CCC3)n1)S(=N)(=O)CCC2]

or is

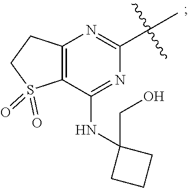 [SMILES: *c1nc2c(c(NC3(CO)CCC3)n1)S(=O)(=O)CC2]

Cy2 is selected from

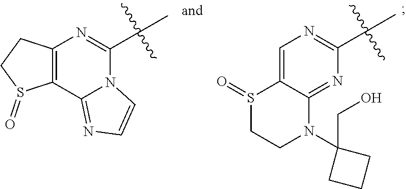 [SMILES: *c1nc2c(c3nccn13)S(=O)CC2 ; *c1ncc2c(n1)N(C1(CO)CCC1)CCS2=O]

Cy4 is selected from

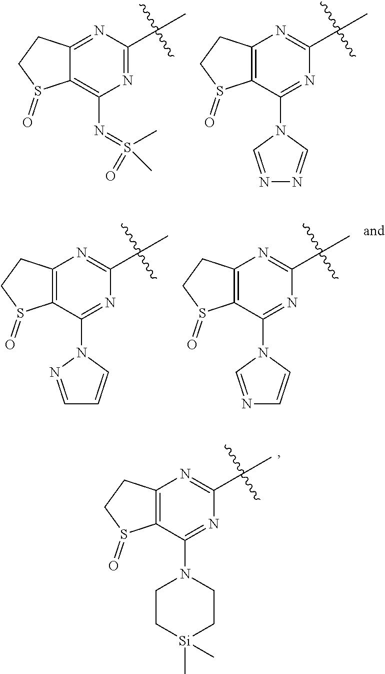 [SMILES: *c1nc2c(c(N=S(C)(C)=O)n1)S(=O)CC2 ; *c1nc2c(c(-n3cnnc3)n1)S(=O)CC2 ; *c1nc2c(c(-n3cccn3)n1)S(=O)CC2 ; *c1nc2c(c(-n3ccnc3)n1)S(=O)CC2 ; *c1nc2c(c(N3CC[Si](C)(C)CC3)n1)S(=O)CC2]

or is

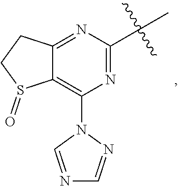 [SMILES: *c1nc2c(c(-n3cncn3)n1)S(=O)CC2]

or is

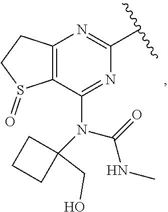 [SMILES: *c1nc2c(c(N(C(=O)NC)C3(CO)CCC3)n1)S(=O)CC2]

or is

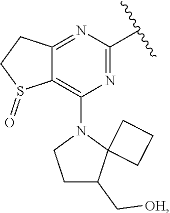 [SMILES: *c1nc2c(c(N3CCC(CO)C34CCC4)n1)S(=O)CC2]

or is

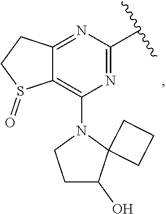 [SMILES: *c1nc2c(c(N3CCC(O)C34CCC4)n1)S(=O)CC2]

or is

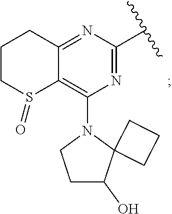 [SMILES: *c1nc2c(c(N3CCC(O)C34CCC4)n1)S(=O)CCC2]

Cy5 is

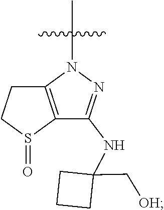 [SMILES: *n1nc(NC2(CO)CCC2)c2c1CCS2=O]

Cy7 is selected from

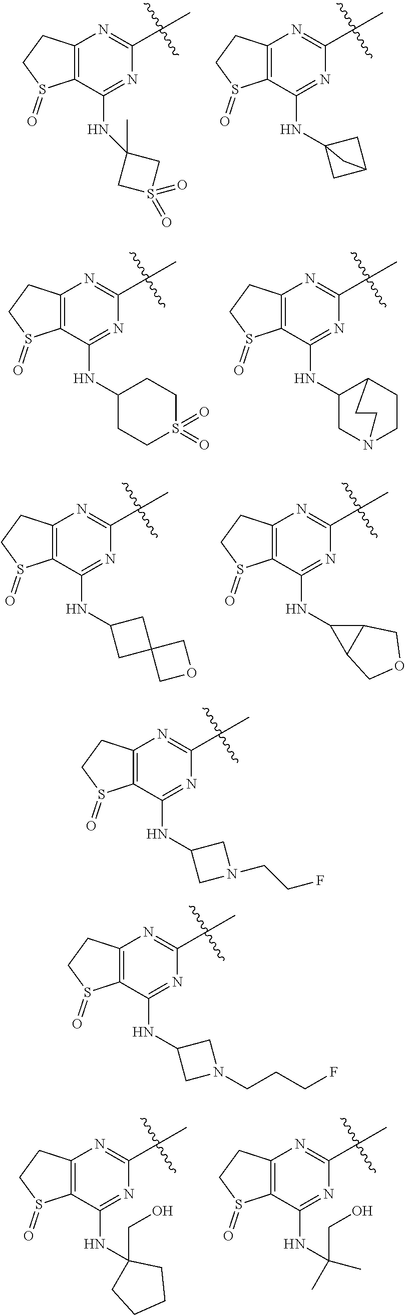

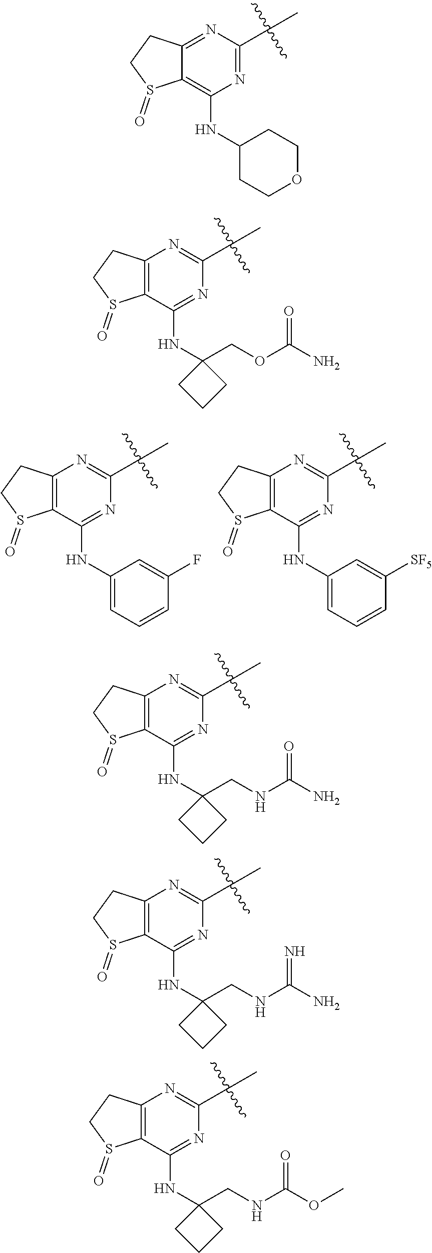

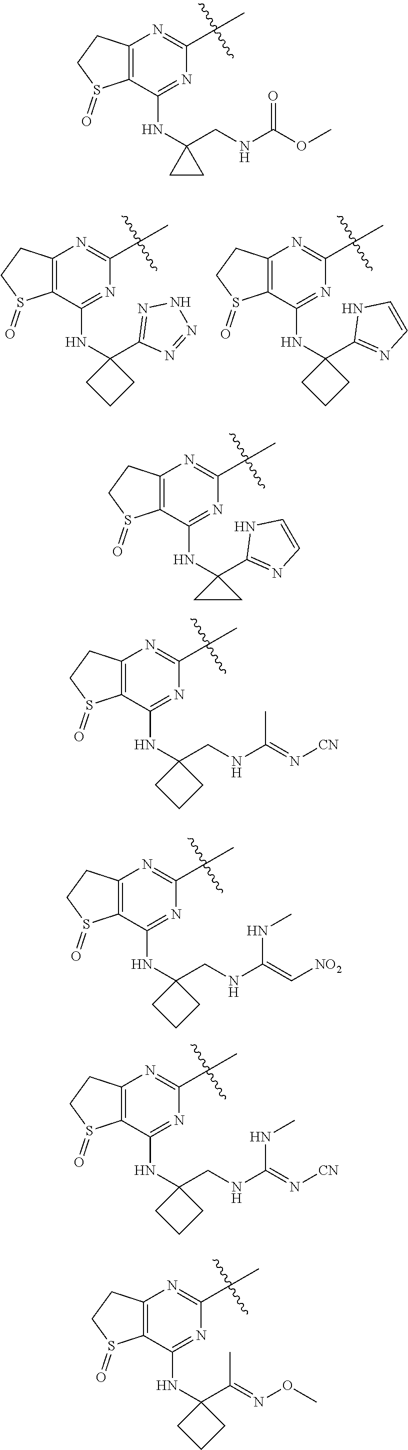

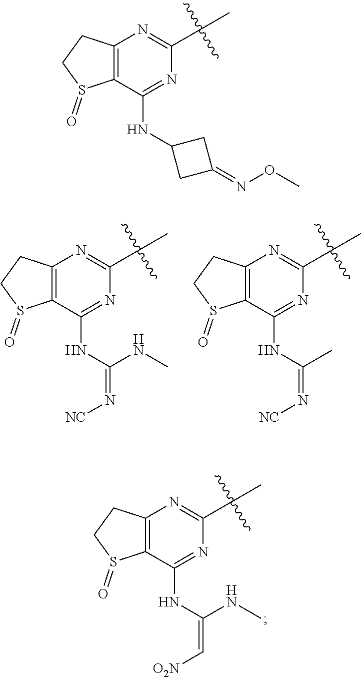

or from

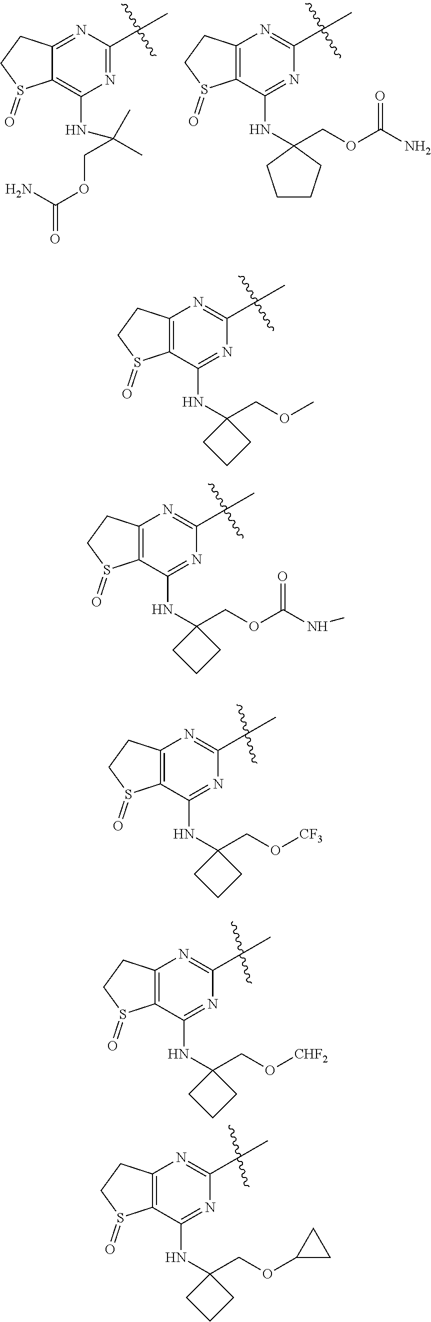

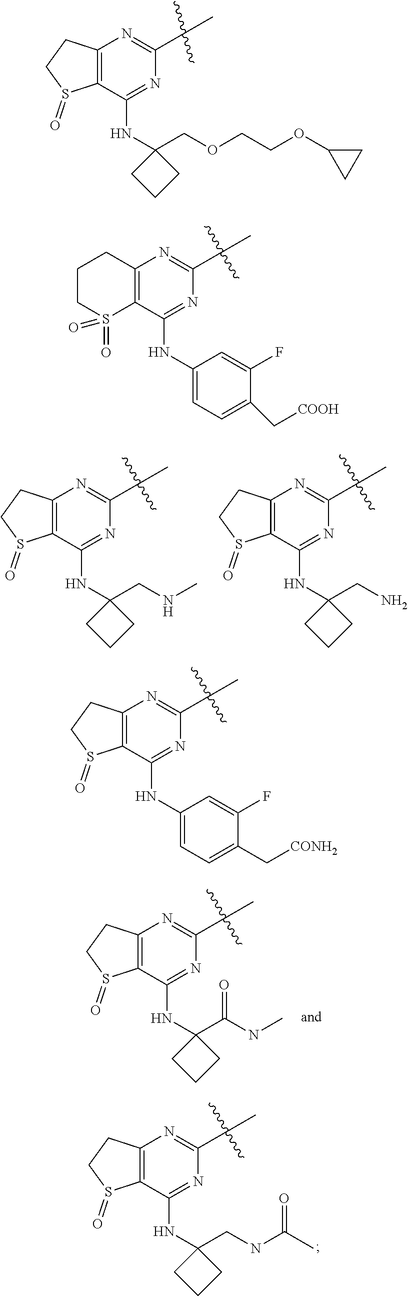

or is

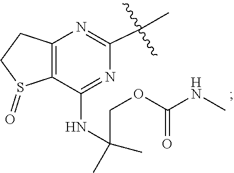 [SMILES: *c1nc2c(c(NC(C)(C)COC(=O)NC)n1)S(=O)CC2]

or is selected from

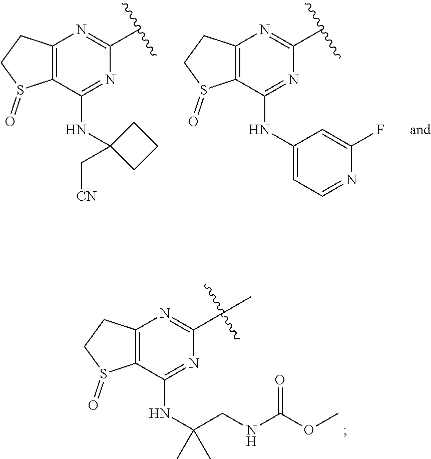

or is

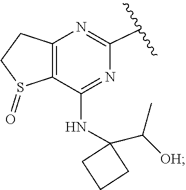 [SMILES: *c1nc2c(c(NC3(C(C)O)CCC3)n1)S(=O)CC2]

or is

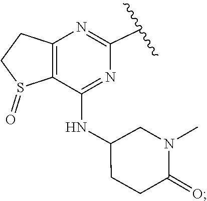 [SMILES: *c1nc2c(c(NC3CCC(=O)N(C)C3)n1)S(=O)CC2]

or is selected from

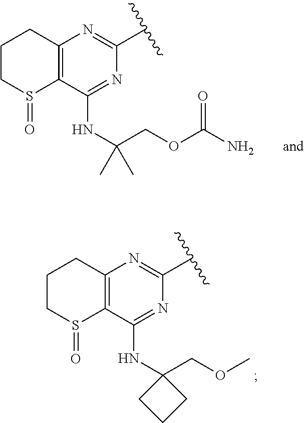 [SMILES: *c1nc2c(c(NC(C)(C)COC(N)=O)n1)S(=O)CCC2 ; *c1nc2c(c(NC3(COC)CCC3)n1)S(=O)CCC2]

or is selected from

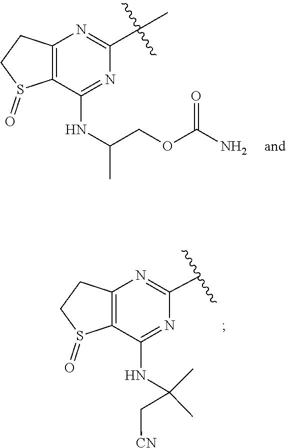 [SMILES: *c1nc2c(c(NC(C)COC(N)=O)n1)S(=O)CC2 ; *c1nc2c(c(NC(C)(C)CC#N)n1)S(=O)CC2]

or is selected from

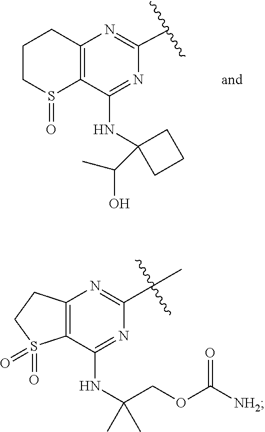 [SMILES: *c1nc2c(c(NC3(C(C)O)CCC3)n1)S(=O)CCC2 ; *c1nc2c(c(NC(C)(C)COC(N)=O)n1)S(=O)(=O)CC2]

and
Cy9 is selected from

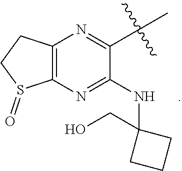 [SMILES: *c1nc2c(nc1NC1(CO)CCC1)S(=O)CC2]

Solution 12 of the present disclosure relates to the compounds, or the stereoisomers or pharmaceutically acceptable salts thereof, as described above in solutions 1-7, wherein Cy1 is selected from

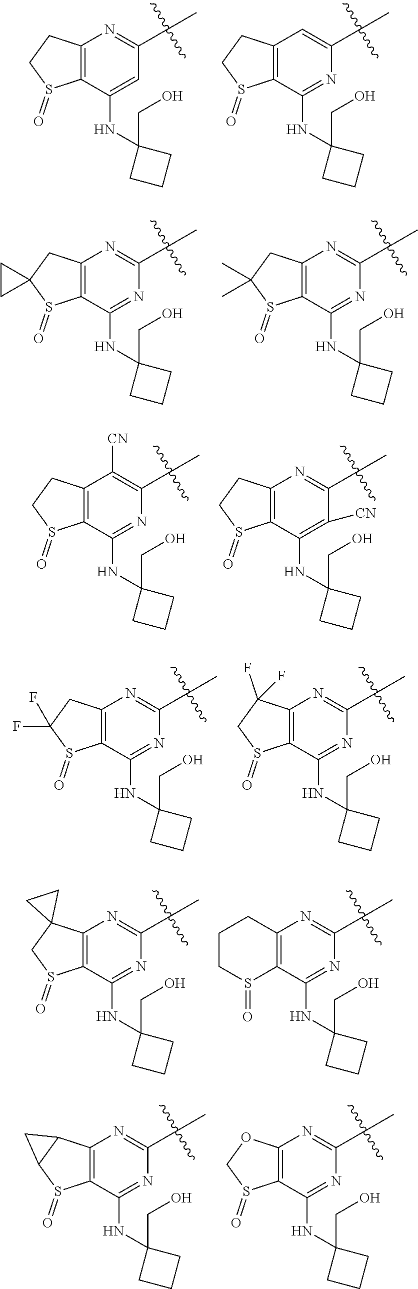

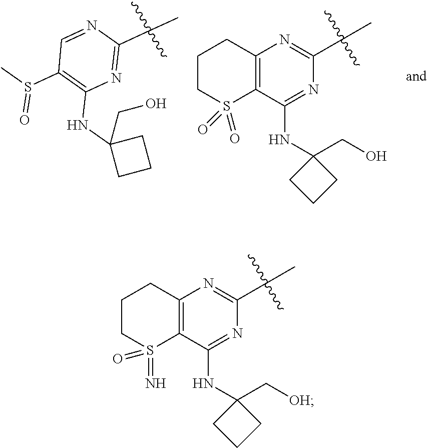 [SMILES: *c1ncc(S(C)=O)c(NC2(CO)CCC2)n1 ; *c1nc2c(c(NC3(CO)CCC3)n1)S(=O)(=O)CCC2 ; *c1nc2c(c(NC3(CO)CCC3)n1)S(=N)(=O)CCC2]

or Cy1 is selected from

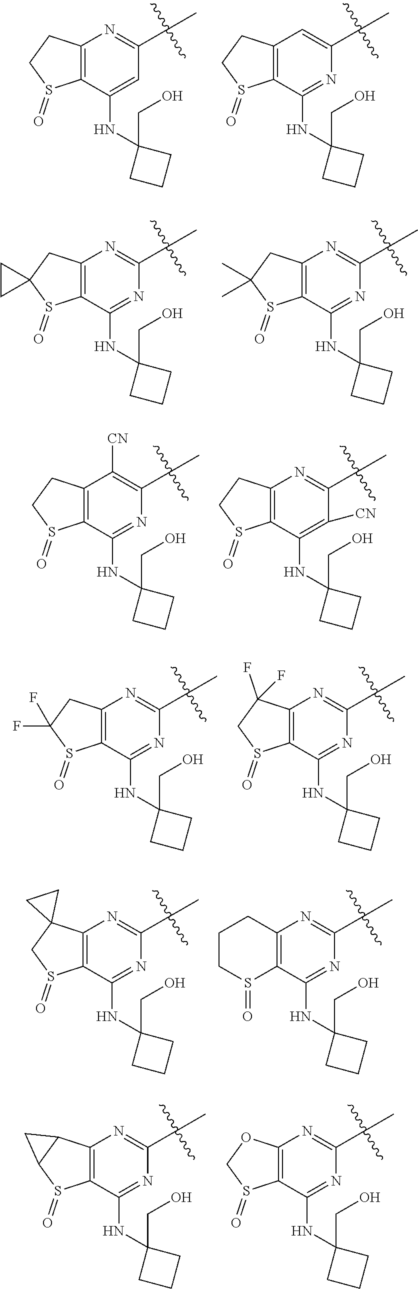

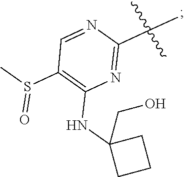 [SMILES: *c1ncc(S(C)=O)c(NC2(CO)CCC2)n1]

or
Cy1 is selected from

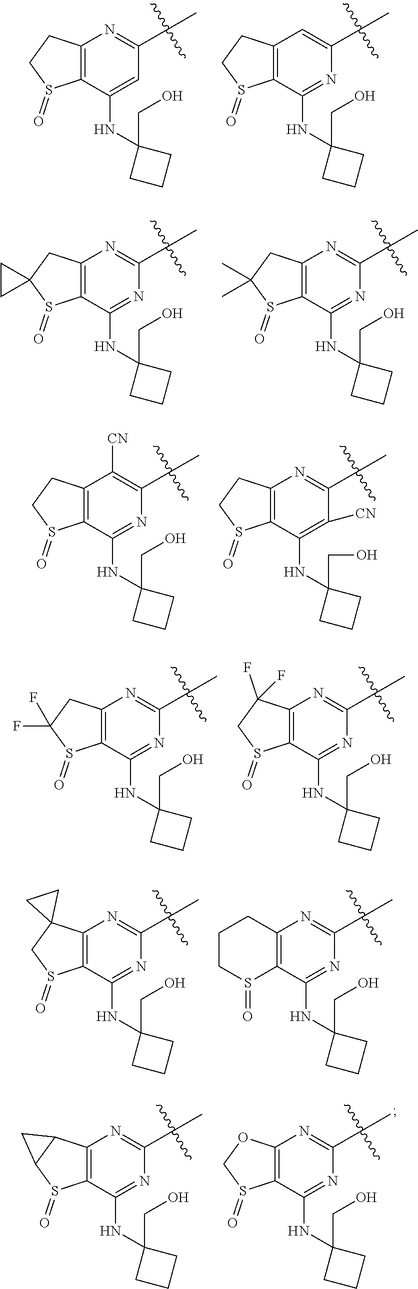

or
Cy1 is

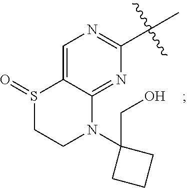 [SMILES: *c1ncc2c(n1)N(C1(CO)CCC1)CCS2=O]

Cy5 is

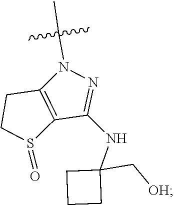 [SMILES: *n1nc(NC2(CO)CCC2)c2c1CCS2=O]

or Cy5 is

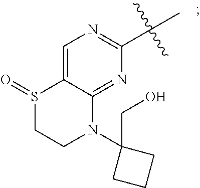 [SMILES: *c1ncc2c(n1)N(C1(CO)CCC1)CCS2=O]

Cy7 is selected from

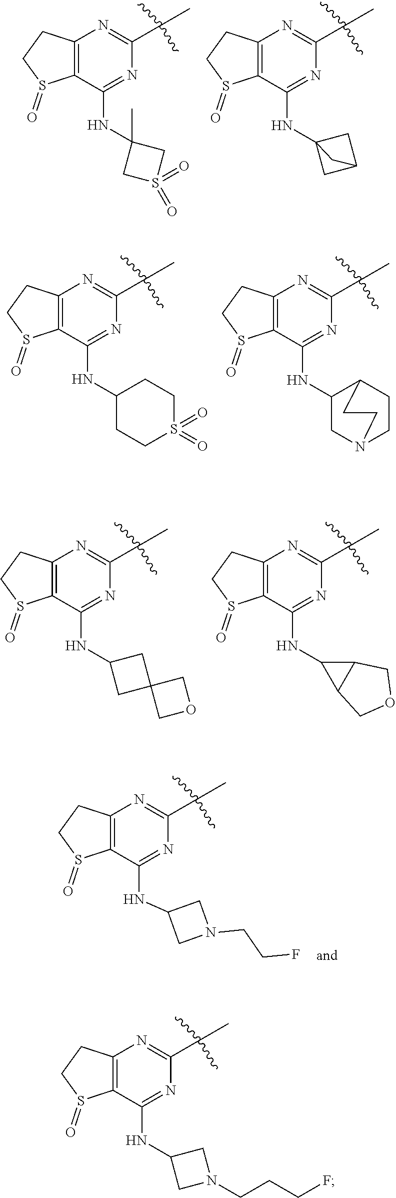

or Cy7 is selected from

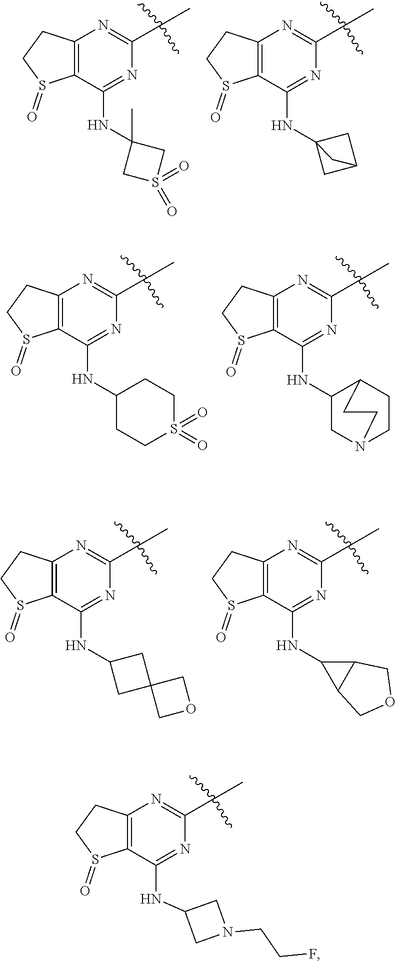

Cy7 is

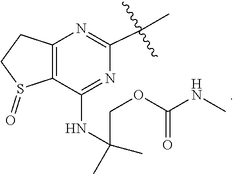 [SMILES: *c1nc2c(c(NC(C)(C)COC(=O)NC)n1)S(=O)CC2]

Solution 13 of the present disclosure relates to the compounds, or the stereoisomers or pharmaceutically acceptable salts thereof, as described above in solutions 1-7, wherein -(L₁)n-(L₂)m- is selected from

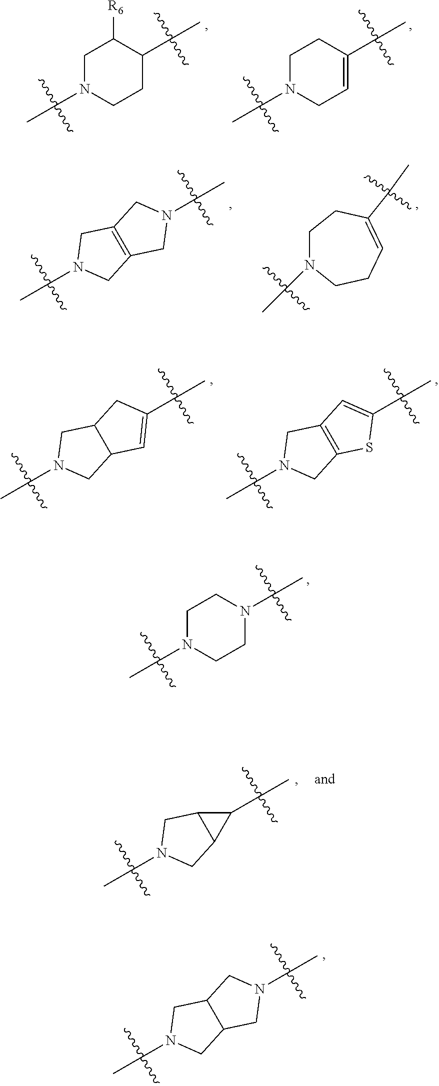

with R₆ being H;
-L₃- is selected from:

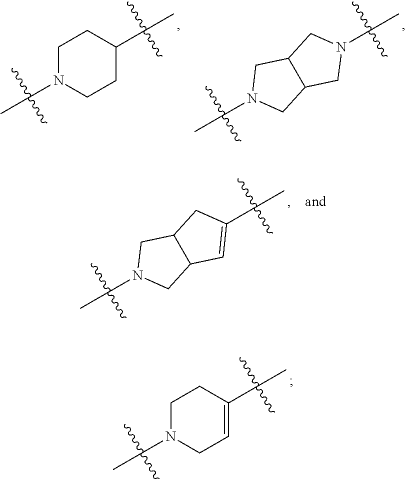 [SMILES: *C1CCN(*)CC1 ; *N1CC2CN(*)CC2C1 ; *C1=CC2CN(*)CC2C1 ; *C1=CCN(*)CC1]

-L₄- is selected from:

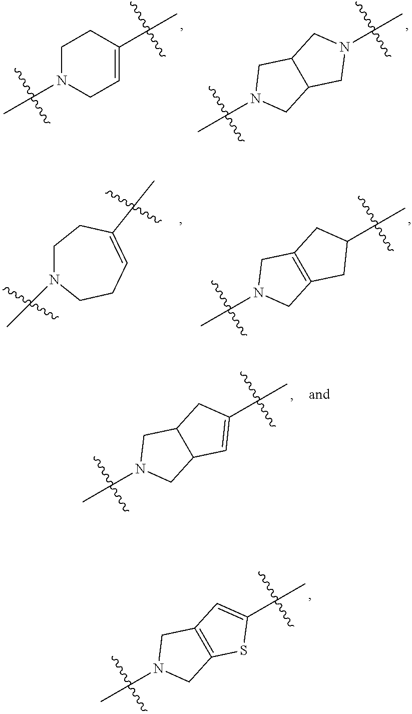 [SMILES: *C1=CCN(*)CC1 ; *N1CC2CN(*)CC2C1 ; *C1=CCCN(*)CC1 ; *C1CC2=C(C1)CN(*)C2 ; *C1=CC2CN(*)CC2C1 ; *c1cc2c(s1)CN(*)C2]

or is

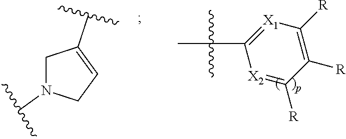

is selected from one of the following structures optionally substituted with 1-3 R_{R}:

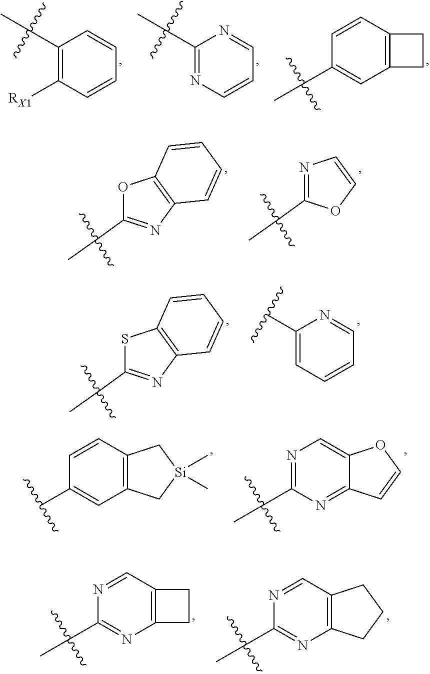

and each R_{R} is independently selected from F, Cl, Br, methyl, isopropyl, ethoxy, methoxy, ethynyl, propynyl, cyclopropyl, cyclobutyl, dimethylamino, and

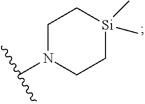 [SMILES: *N1CC[Si](C)(C)CC1]

and in some embodiments,

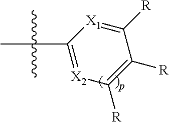

is selected from one of the following structures optionally substituted with 1-3 R_{R}:

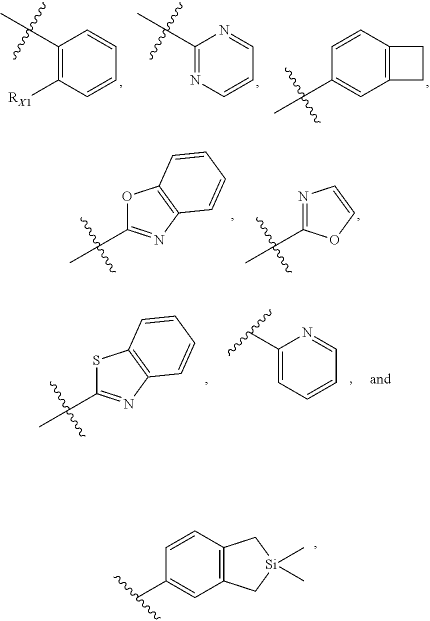

each R_{R} is independently selected from F, Cl, Br, methyl, ethynyl, propynyl, cyclopropyl,

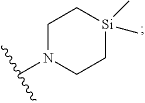 [SMILES: *N1CC[Si](C)(C)CC1]

R_{X1} is H;
in some embodiments,

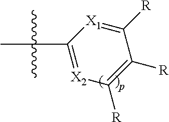

is selected from one of the following structures optionally substituted with 1-3 R_{R}:

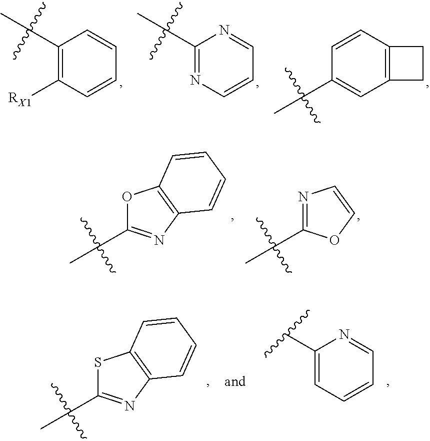

and each R_{R} is independently selected from F, Cl, Br, methyl, ethynyl, propynyl, or cyclopropyl; R_{X1} is H;
in some embodiments,

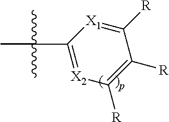

is selected from one of the following structures optionally substituted with 1-3 R_{R}:

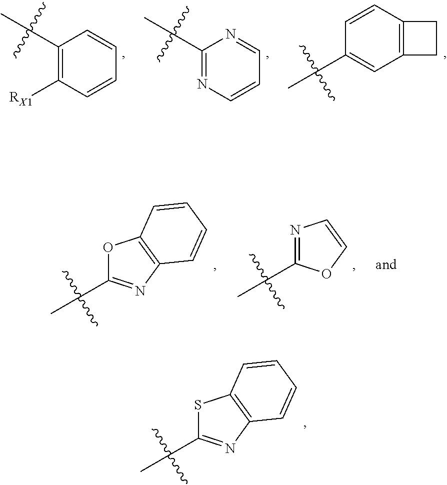

and each R_{R} is independently selected from F, Cl, Br, methyl, ethynyl, propynyl, or cyclopropyl; R_{X1} is H;
or R_{X1} and R₆, together with the atom to which they are attached, form morpholinyl, furyl, pyrrolyl, or thienyl; in some embodiments, R_{X1} and R₆, together with the atom to which they are attached, form morpholinyl, furyl, or thienyl.

Solution 14 of the present disclosure relates to the compounds, or stereoisomers or pharmaceutically acceptable salts thereof, as described above in solutions 1-7, wherein -(L₁)n-(L₂)m- is selected from

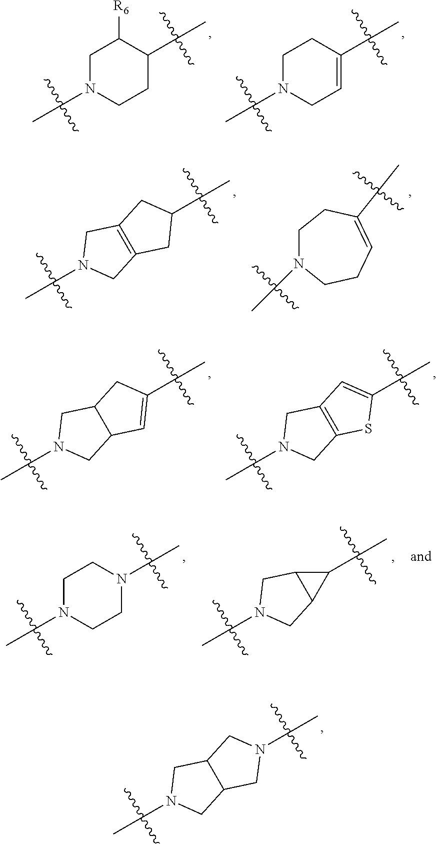

with R₆ being H;
-L₃- is selected from:

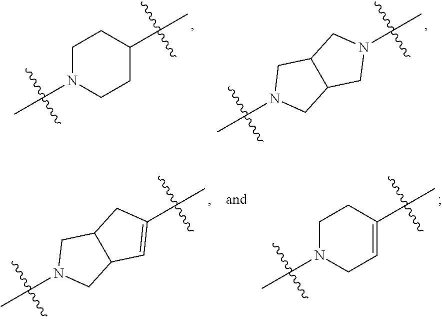 [SMILES: *C1CCN(*)CC1 ; *N1CC2CN(*)CC2C1 ; *C1=CC2CN(*)CC2C1 ; *C1=CCN(*)CC1]

-L₄- is selected from:

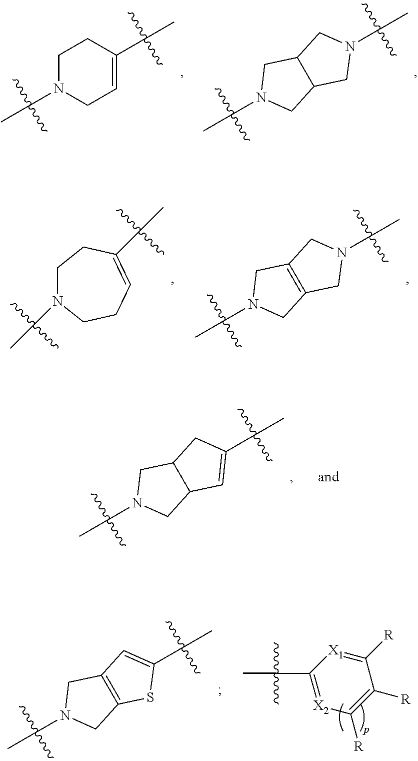

is selected from one of the following structures optionally substituted with 1-3 R_{R}:

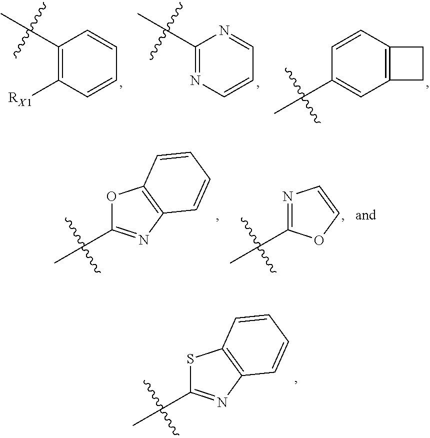

and each R_{R} is independently selected from F, Cl, Br, methyl, ethynyl, or cyclopropyl; R_{X1} is H; and
or R_{X1} and R₆, together with the atom to which they are attached, form morpholinyl, furyl, or thienyl.

Solution 15 of the present disclosure relates to the compounds, or the stereoisomers or pharmaceutically acceptable salts thereof, as described above in solutions 1-5,

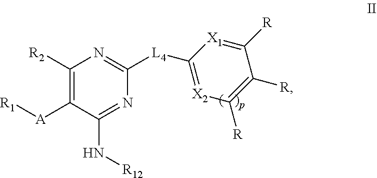

wherein L₄ is

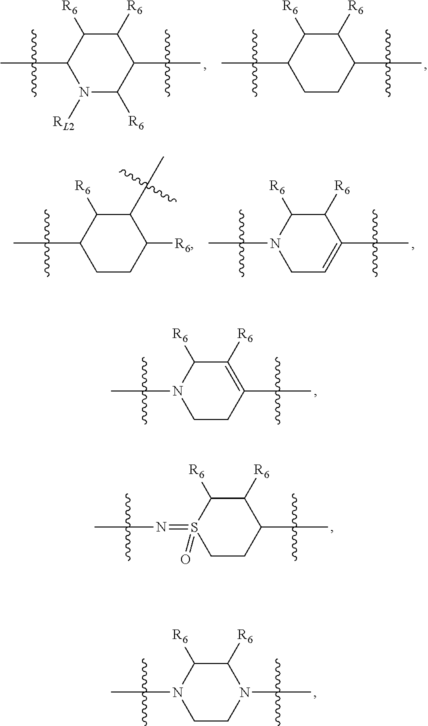

a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 6-membered heterocycloalkyl fused 5- to 6-membered heterocycloalkyl containing 1-3 heteroatoms selected from N, S, and O, a 5- to 6-membered heterocycloalkyl fused 5- to 6-membered cycloalkyl containing 1-3 heteroatoms selected from N, S, and O, or a 7- to 12-membered spiro heterocycloalkyl containing 1-3 heteroatoms selected from N, S, and O, wherein the monocyclic heterocycloalkyl, 5- to 6-membered heterocycloalkyl fused 5- to 6-membered heterocycloalkyl, 5- to 6-membered heterocycloalkyl fused 5- to 6-membered cycloalkyl, and spiro heterocycloalkyl are optionally substituted with 1-3 groups selected from halogen, ═O, CN, C₁₋₄ alkyl, C₁₋₄ alkoxy, OH, and NH₂;
A is —S(O)—, —C(O)—, —B(OH)—, —S—, —S(═N)—CN, or —C(═N)—CN;
X₁ and X₂ are independently CR_{X1}, S, or N;
R_{L2} is H, C₁₋₄ alkyl, or C₃₋₆ cycloalkyl;
R₆ is H, halogen, ═O, CN, C₁₋₄ alkyl, or C₁₋₄ alkoxy;
each R_{X1} and R are independently H, halogen, C₁₋₄ alkyl, C₁₋₄ alkoxy, —N(CH₃)₂, —NH(CH₃), C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, O, and Si, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₁ and R₂, together with the atom to which they are attached, form a 5- to 10-membered heterocycloalkyl or a 5- to 6-membered heteroaryl containing 1-3 heteroatoms selected from N, S, B, and O, wherein the heterocycloalkyl and heteroaryl are optionally substituted with 1-3 groups selected from halogen, ═O, CN, C₁₋₄ alkyl, C₁₋₄ alkoxy, OH, and NH₂;
R₁₂ is selected from a 4- to 10-membered heterocycloalkylene, a 3- to 10-membered cycloalkylene, a 6- to 10-membered arylene, a 5- to 10-membered heteroarylene, hydroxy-C₁₋₆ alkyl, C₁₋₄ alkyl-CN, —CRaRb=N—O—C₁₋₄ alkyl, —C(NRaRb)=N—CN, —C(C₁₋₄ alkyl)=N—CN, —C(NRaRb)=CRa—NO₂, —C(CH₃)₂—CH₂—O—C(O)NH₂, —C(CH₃)₂—CH₂—NH—C(O)O(C₁₋₄ alkyl), —C(CH₃)₂—CH₂—O—C(O)NHCH₃, —CH(CH₃)—CH₂—O—C(O)NH₂, —CH(CH₃)—CH₂—NH—C(O)O(C₁₋₄ alkyl), —CH(CH₃)—CH₂—O—C(O)NHCH₃, —C(O)—Ra, or —C(O)-(4- to 6-membered heterocycloalkyl), wherein the heterocycloalkylene, cycloalkylene, arylene, and heteroarylene are optionally further substituted with 1-3 groups selected from halogen, CN, ═O, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NRa—C(O)NH₂, —(CH₂)ₜ—NRa—C(═NH)NH₂, —(CH₂)ₜ—NRa—C(O)—O—(C₁₋₄ alkyl), —(CH₂)ₜ—NRa—C(C₁₋₄ alkyl)=N—CN, —(CH₂)ₜ—NRa—C(NRaRb)=N—CN, —(CH₂)ₜ—NRa—C(NRaRb)=CRa—NO₂, —(CH₂)ₜ—C(NRaRb)=N—CN, —(CH₂)ₜ—C(C₁₋₄ alkyl)=N—CN, —(CH₂)ₜ—C(C₁₋₄ alkyl)=N—O—(C₁₋₄ alkyl), ═N—O—C₁₋₄ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃;
Ra and Rb are each independently selected from H, halogen, C₁₋₄ alkyl, C₁₋₄ haloalkyl, C₁₋₄ alkoxy, halo C₁₋₄ alkoxy, or a 4- to 6-membered heterocycloalkyl containing 1-2 heteroatoms selected from N and O; and
p is selected from 0 or 1.

Solution 16 of the present disclosure relates to the compound, or the stereoisomer or pharmaceutically acceptable salt thereof, as described above in solution 15,
wherein L₄ is

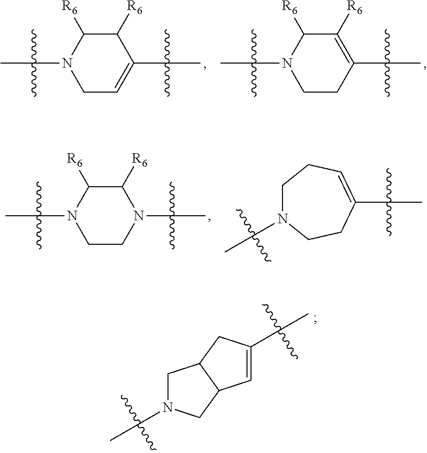

and R₁₂ is selected from —C(CH₃)₂—CH₂—O—C(O)NH₂, —C(CH₃)₂—CH₂—NH—C(O)O(C₁₋₄ alkyl), —C(CH₃)₂—CH₂—O—C(O)NHCH₃, —CH(CH₃)—CH₂—O—C(O)NH₂, —CH(CH₃)—CH₂—NH—C(O)O(C₁₋₄ alkyl), and —CH(CH₃)—CH₂—O—C(O)NHCH₃.

Solution 17 of the present disclosure relates to the compounds as described above in formulas (I) and (II), or stereoisomers or pharmaceutically acceptable salts thereof, wherein
L₄ is

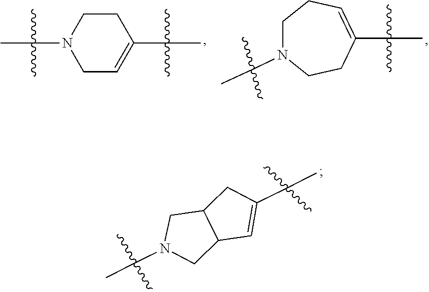 [SMILES: *C1=CCN(*)CC1 ; *C1=CCCN(*)CC1 ; *C1=CC2CN(*)CC2C1]

or L₄ is

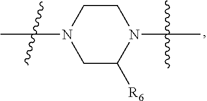

R₆ and R_{X1}, together with the atom to which they are attached, form a 5- to 7-membered heterocycloalkyl containing 1-3 heteroatoms selected from N, S, O, and Si;
A is —S(O)—;
R₁ and R₂, together with the atom to which they are attached, form

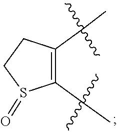 [SMILES: *C1=C(*)S(=O)CC1]

R₁₂ is selected from —C(CH₃)₂—CH₂—O—C(O)NH₂, —C(CH₃)₂—CH₂—NH—C(O)O(C₁₋₂ alkyl), —C(CH₃)₂—CH₂—O—C(O)NHCH₃, —CH(CH₃)—CH₂—O—C(O)NH₂, —CH(CH₃)—CH₂—NH—C(O)O(C₁₋₂ alkyl), and —CH(CH₃)—CH₂—O—C(O)NHCH₃;

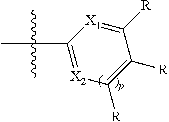

is selected from one of the following structures optionally substituted with 1-3 R_{R}:

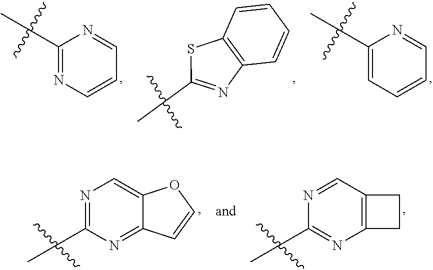 [SMILES: *c1ncccn1 ; *c1nc2ccccc2s1 ; *c1ccccn1 ; *c1ncc2occc2n1 ; *c1ncc2c(n1)CC2]

and each R_{R} is independently selected from F, Cl, methyl, ethyl, isopropyl, ethoxy, methoxy, ethynyl, propynyl, cyclopropyl, cyclobutyl, and dimethylamino.

Solution 18 of the present disclosure relates to the compound as described above in formula (I), or the stereoisomer or pharmaceutically acceptable salt thereof, wherein
Cy is selected from Cy1, Cy2, Cy4, Cy5, Cy7, and Cy9;
when Cy is selected from Cy1 and Cy2, L is -(L₁)n-(L₂)m-; when Cy is selected from Cy4 and Cy5, L is -L₃-; and when Cy is selected from Cy7 and Cy9, L is -L₄-;
Cy1 is selected from

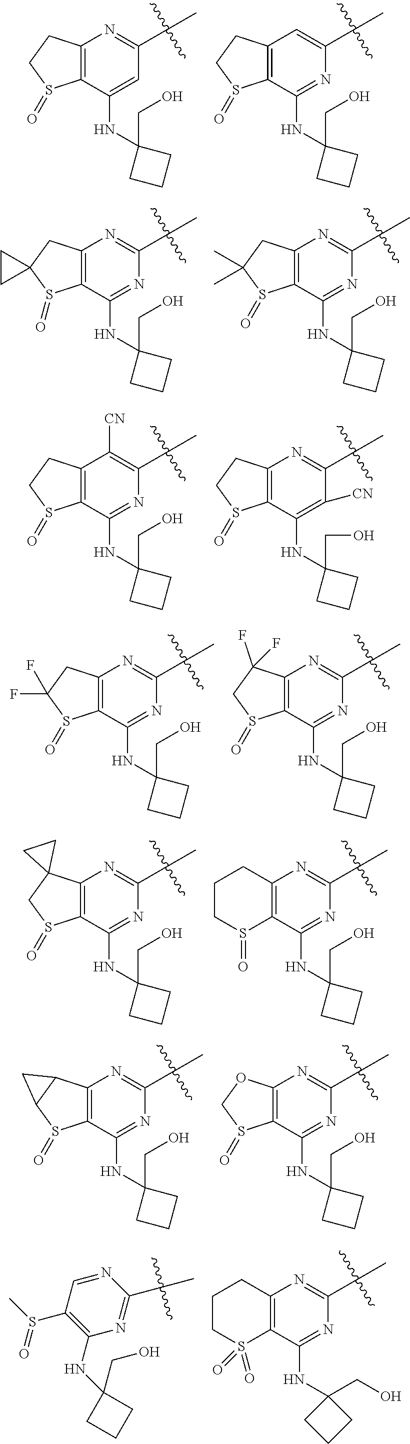

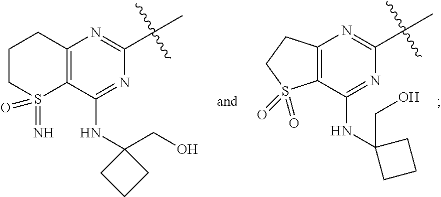 [SMILES: *c1nc2c(c(NC3(CO)CCC3)n1)S(=N)(=O)CCC2 ; *c1nc2c(c(NC3(CO)CCC3)n1)S(=O)(=O)CC2]

Cy2 is selected from

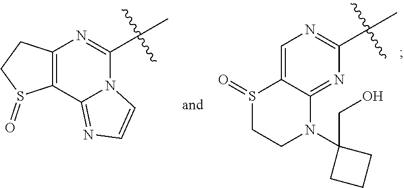 [SMILES: *c1nc2c(c3nccn13)S(=O)CC2 ; *c1ncc2c(n1)N(C1(CO)CCC1)CCS2=O]

Cy4 is selected from

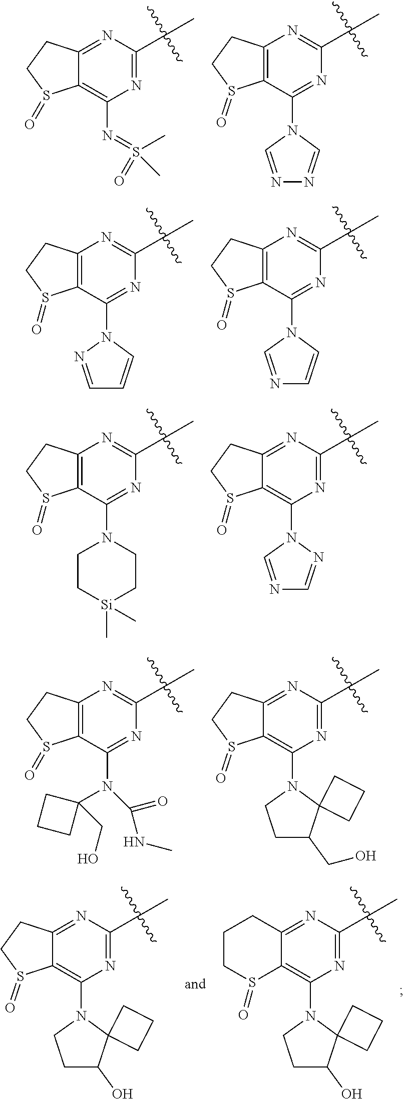

Cy5 is

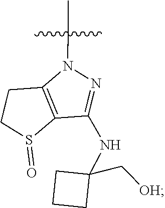 [SMILES: *n1nc(NC2(CO)CCC2)c2c1CCS2=O]

Cy7 is selected from

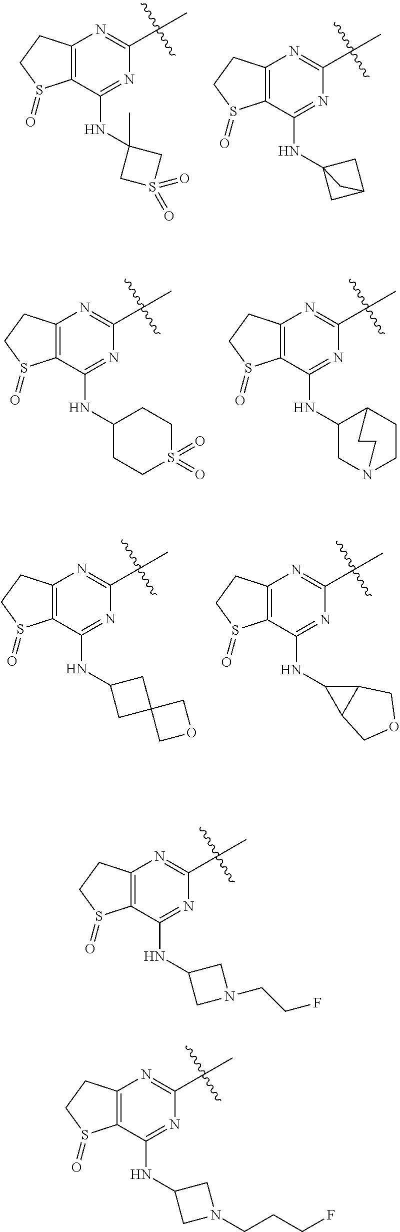

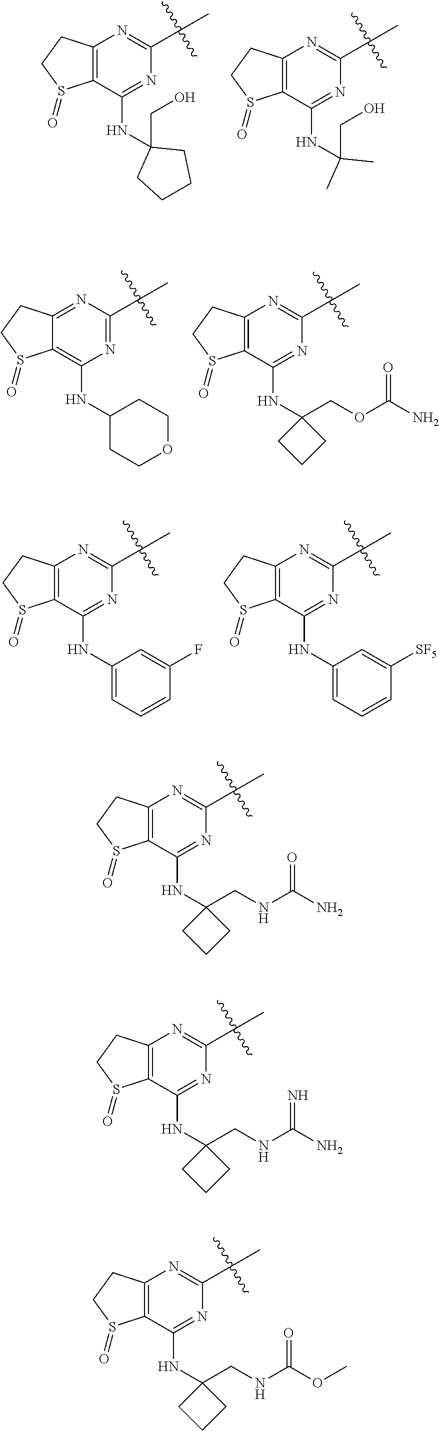

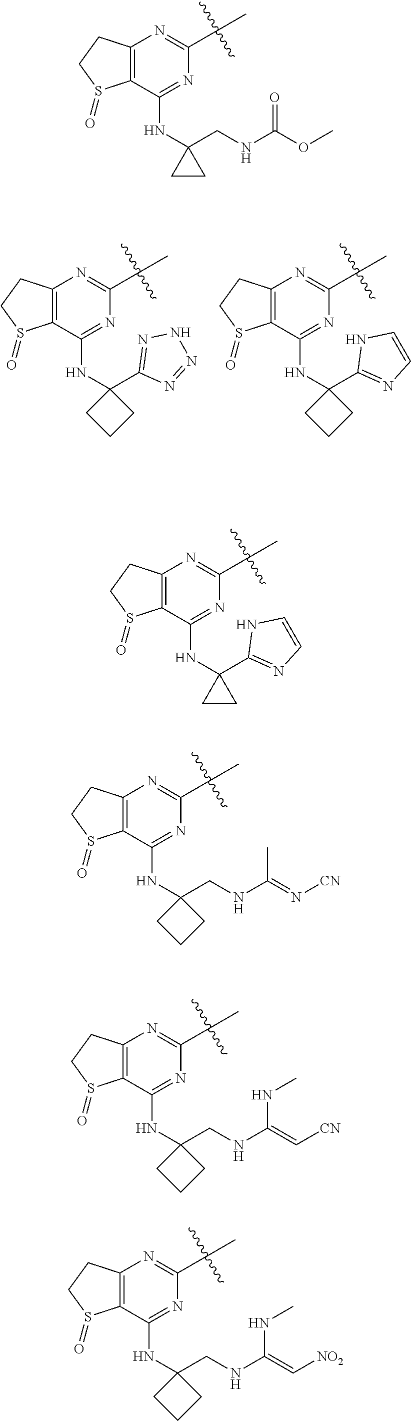

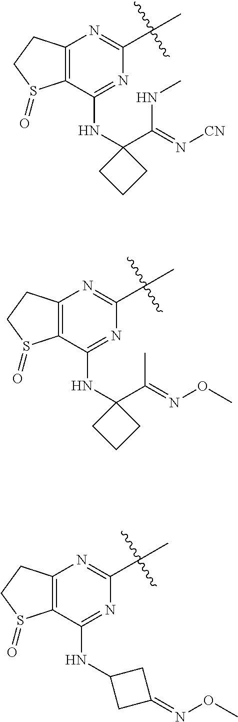 [SMILES: *c1nc2c(c(NC3(/C(=N/C#N)NC)CCC3)n1)S(=O)CC2 ; *c1nc2c(c(NC3(/C(C)=N/OC)CCC3)n1)S(=O)CC2 ; *c1nc2c(c(NC3CC(=NOC)C3)n1)S(=O)CC2]

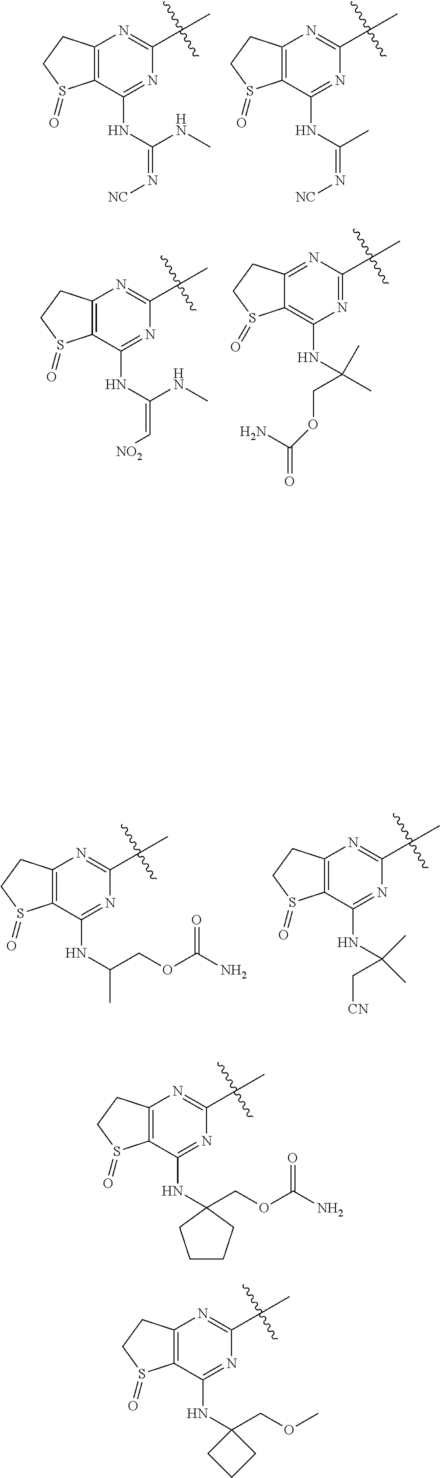

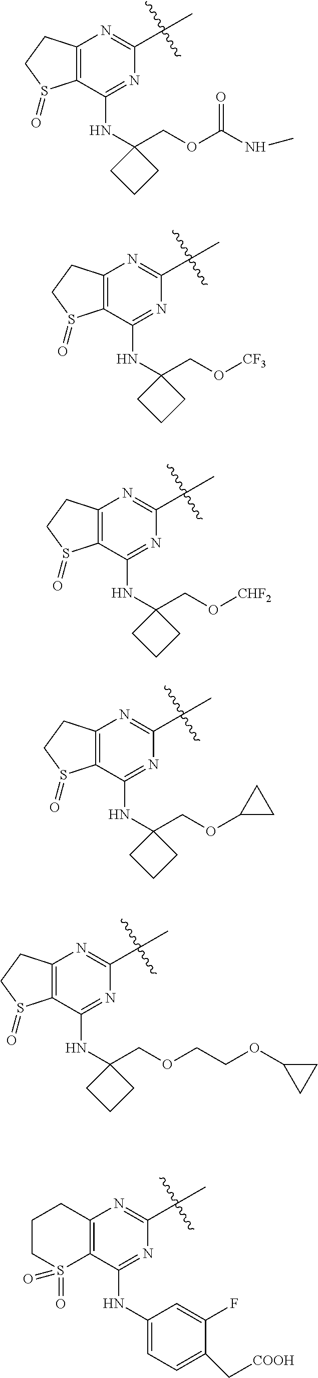 [SMILES: *c1nc2c(c(NC3(COC(=O)NC)CCC3)n1)S(=O)CC2 ; *c1nc2c(c(NC3(COC(F)(F)F)CCC3)n1)S(=O)CC2 ; *c1nc2c(c(NC3(COC(F)F)CCC3)n1)S(=O)CC2 ; *c1nc2c(c(NC3(COC4CC4)CCC3)n1)S(=O)CC2 ; *c1nc2c(c(NC3(COCCOC4CC4)CCC3)n1)S(=O)CC2 ; *c1nc2c(c(Nc3ccc(CC(=O)O)c(F)c3)n1)S(=O)(=O)CCC2]

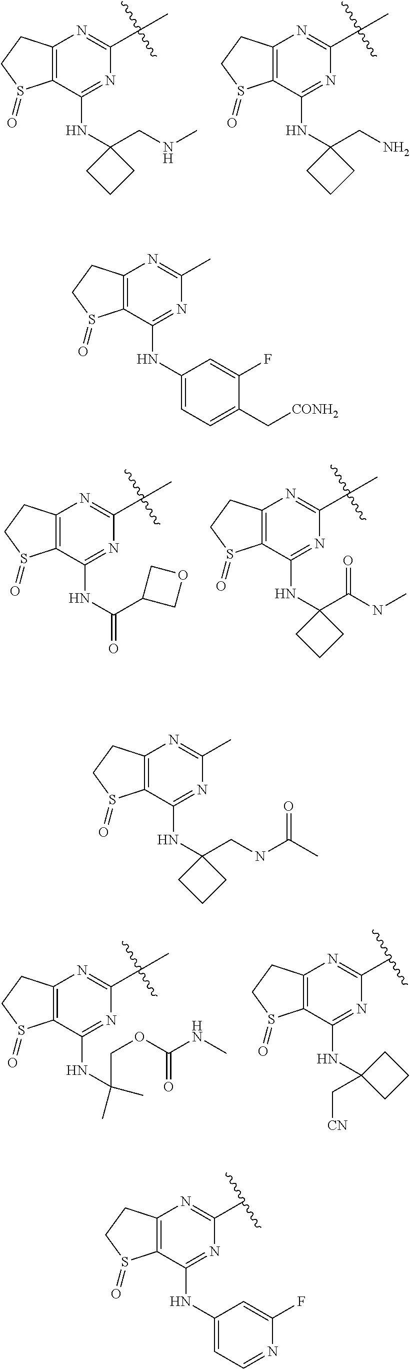

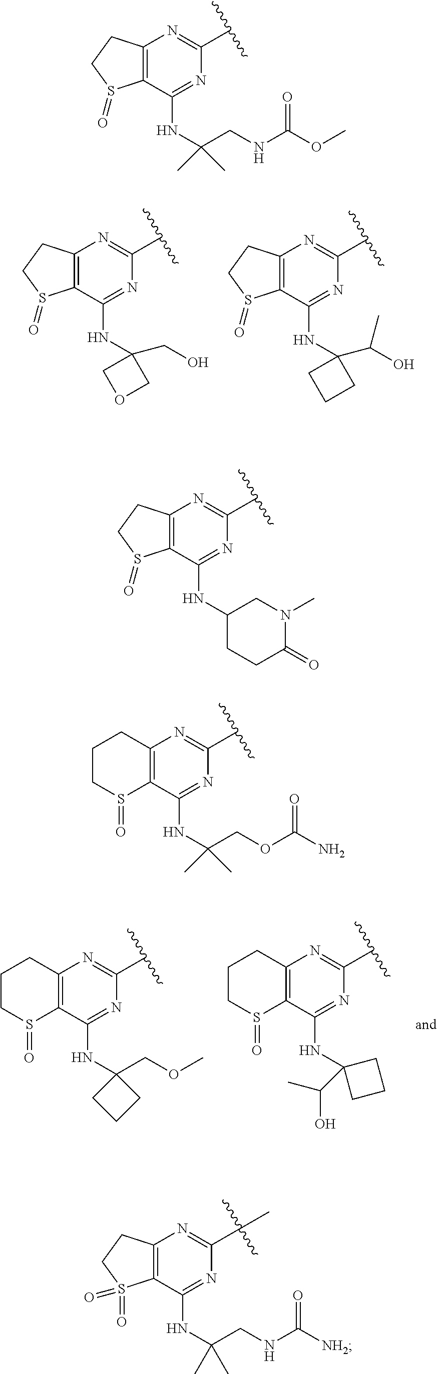

Cy9 is

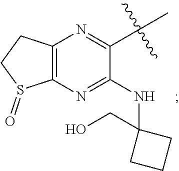 [SMILES: *c1nc2c(nc1NC1(CO)CCC1)S(=O)CC2]

-(L₁)n-(L₂)m- is selected from:

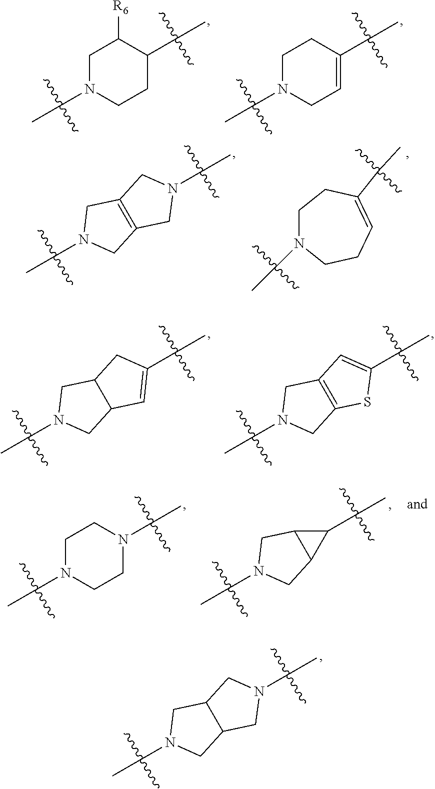

with R₆ being H;
-L₃- is selected from:

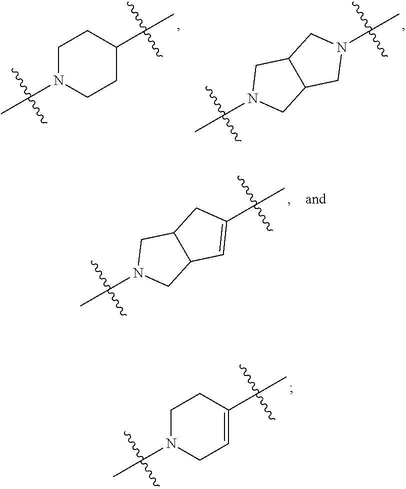 [SMILES: *C1CCN(*)CC1 ; *N1CC2CN(*)CC2C1 ; *C1=CC2CN(*)CC2C1 ; *C1=CCN(*)CC1]

-L₄- is selected from:

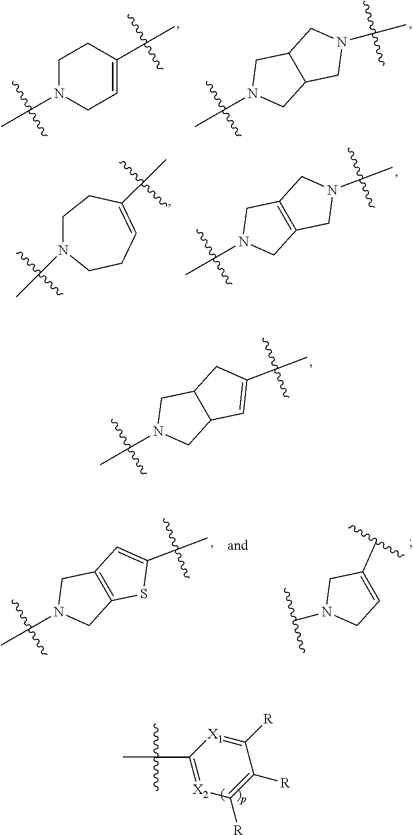

is selected from one of the following structures optionally substituted with 1-3 R_{R}:

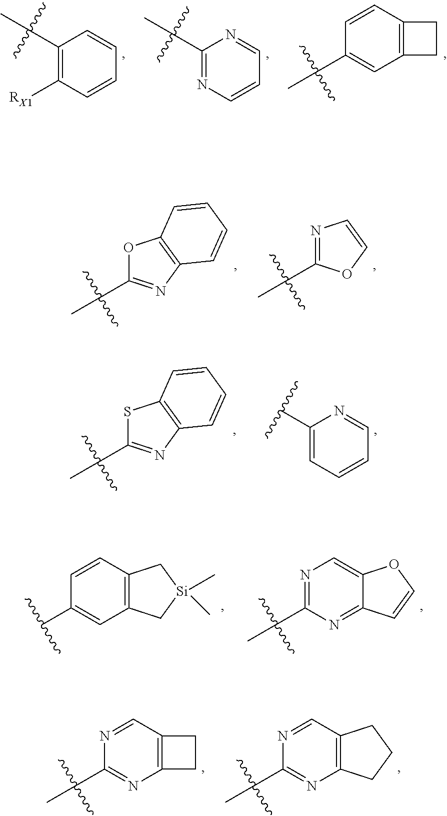

and each R_{R} is independently selected from F, Cl, Br, methyl, isopropyl, ethoxy, methoxy, ethynyl, propynyl, cyclopropyl, cyclobutyl, dimethylamino, and

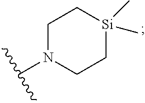 [SMILES: *N1CC[Si](C)(C)CC1]

and R_{X1} is H;
or R_{X1} and R₆, together with the atom to which they are attached, form morpholinyl, furyl, pyrrolyl, or thienyl.

Solution 19 of the present disclosure relates to the compound as described above in formula (I), or the stereoisomer or pharmaceutically acceptable salt thereof, wherein
Cy is selected from Cy1 and Cy7;
when Cy is selected from Cy1, L is -(L₁)n-(L₂)m-; and when Cy is Cy7, L is -L₄-;
Cy1 is selected from

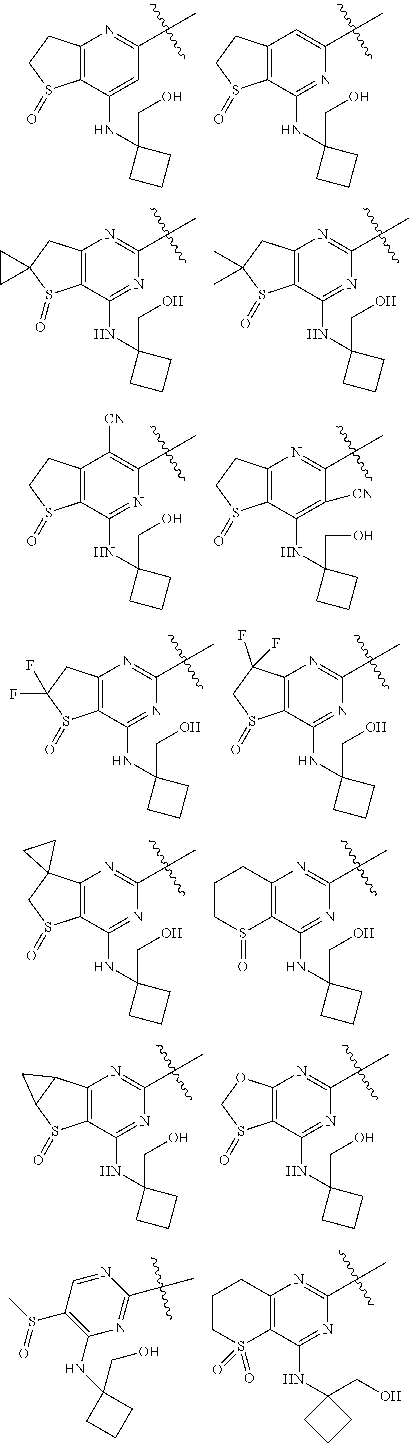

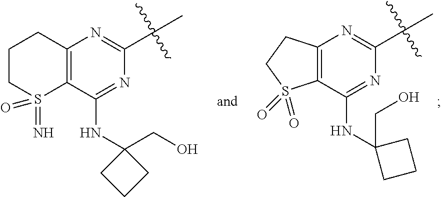 [SMILES: *c1nc2c(c(NC3(CO)CCC3)n1)S(=N)(=O)CCC2 ; *c1nc2c(c(NC3(CO)CCC3)n1)S(=O)(=O)CC2]

Cy7 is selected from

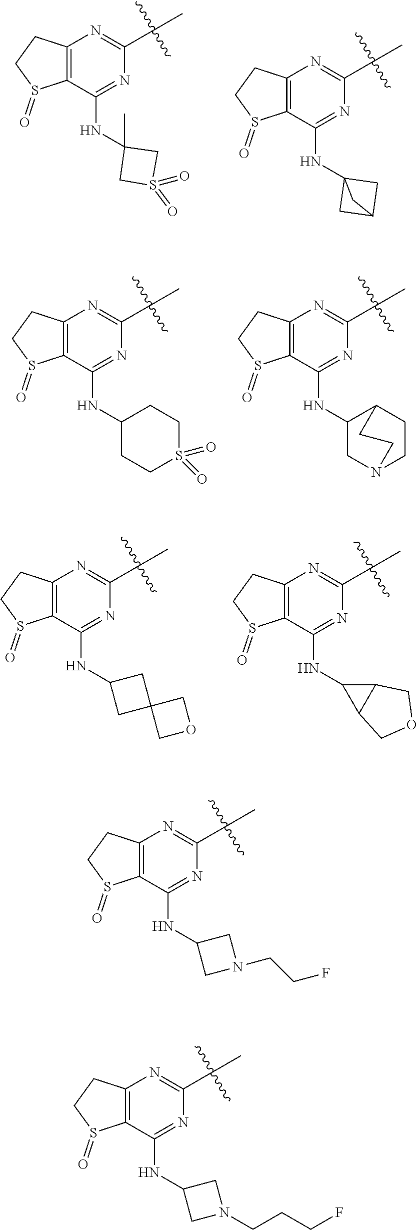

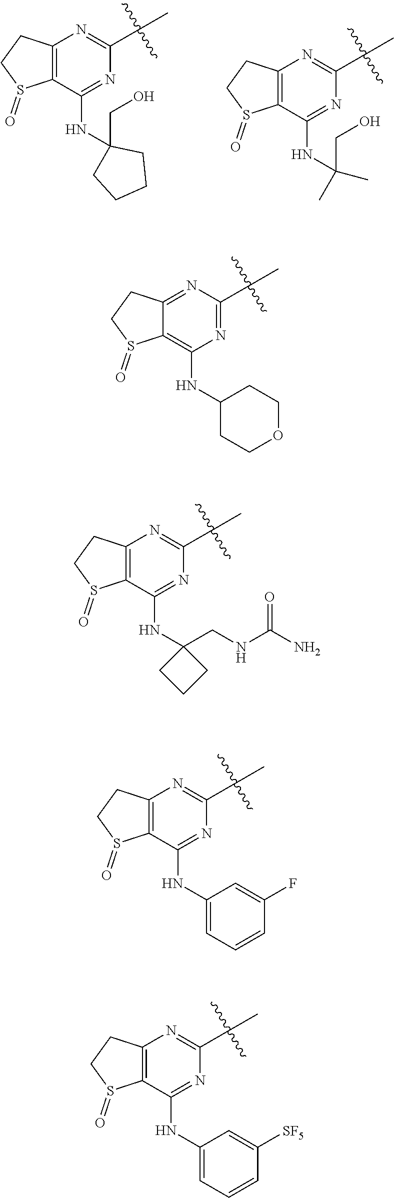

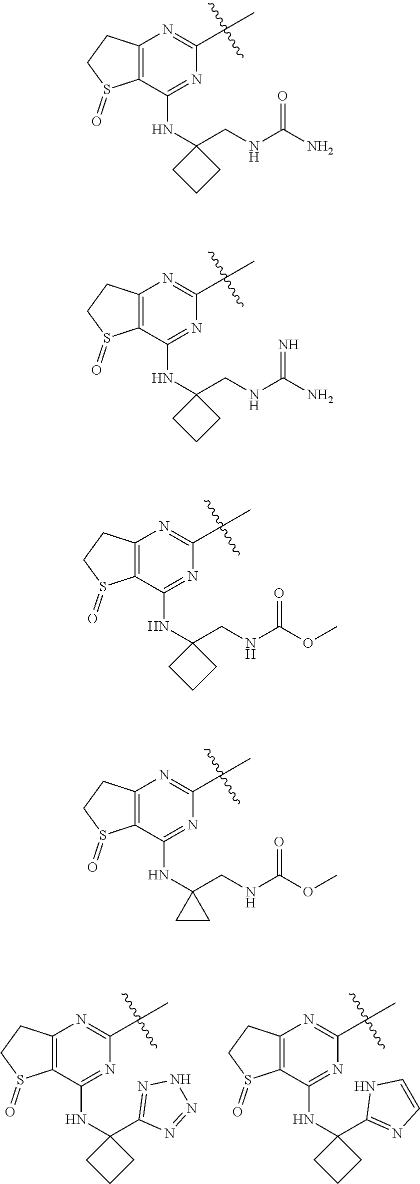

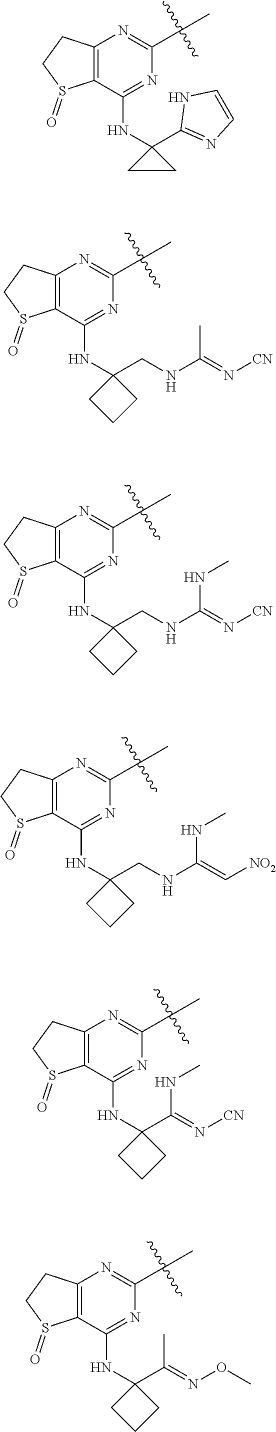 [SMILES: *c1nc2c(c(NC3(c4ncc[nH]4)CC3)n1)S(=O)CC2 ; *c1nc2c(c(NC3(CN/C(C)=N/C#N)CCC3)n1)S(=O)CC2 ; *c1nc2c(c(NC3(CN/C(=N/C#N)NC)CCC3)n1)S(=O)CC2 ; *c1nc2c(c(NC3(CN/C(=C/[N+](=O)[O-])NC)CCC3)n1)S(=O)CC2 ; *c1nc2c(c(NC3(/C(=N/C#N)NC)CCC3)n1)S(=O)CC2 ; *c1nc2c(c(NC3(/C(C)=N/OC)CCC3)n1)S(=O)CC2]

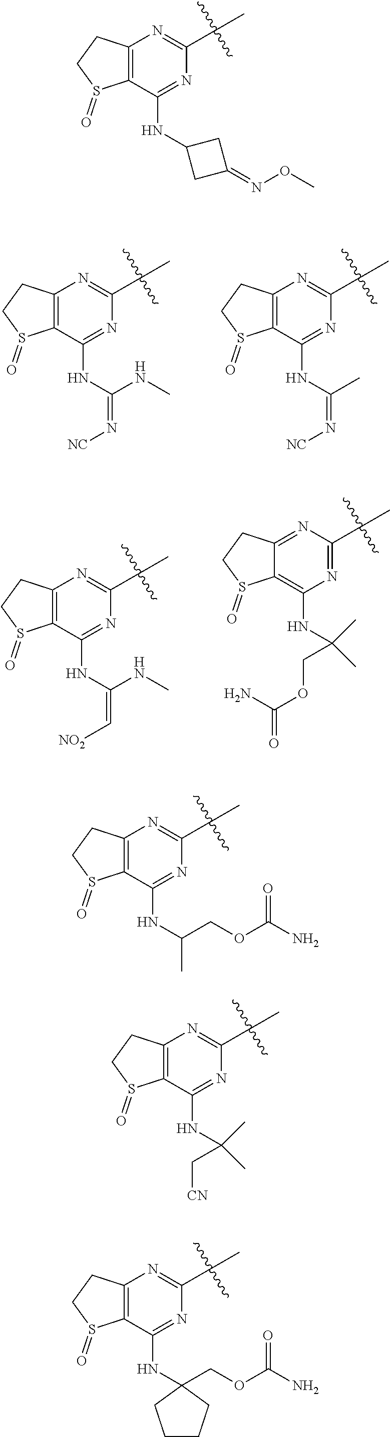

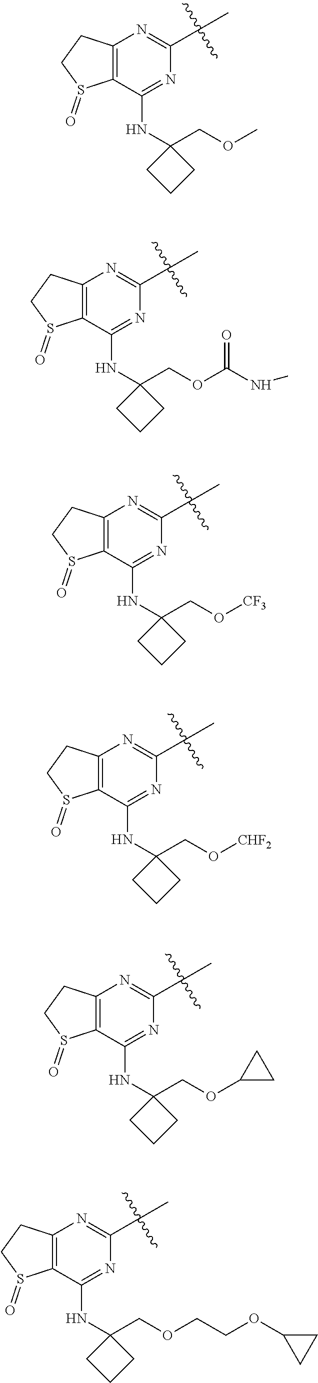 [SMILES: *c1nc2c(c(NC3(COC)CCC3)n1)S(=O)CC2 ; *c1nc2c(c(NC3(COC(=O)NC)CCC3)n1)S(=O)CC2 ; *c1nc2c(c(NC3(COC(F)(F)F)CCC3)n1)S(=O)CC2 ; *c1nc2c(c(NC3(COC(F)F)CCC3)n1)S(=O)CC2 ; *c1nc2c(c(NC3(COC4CC4)CCC3)n1)S(=O)CC2 ; *c1nc2c(c(NC3(COCCOC4CC4)CCC3)n1)S(=O)CC2]

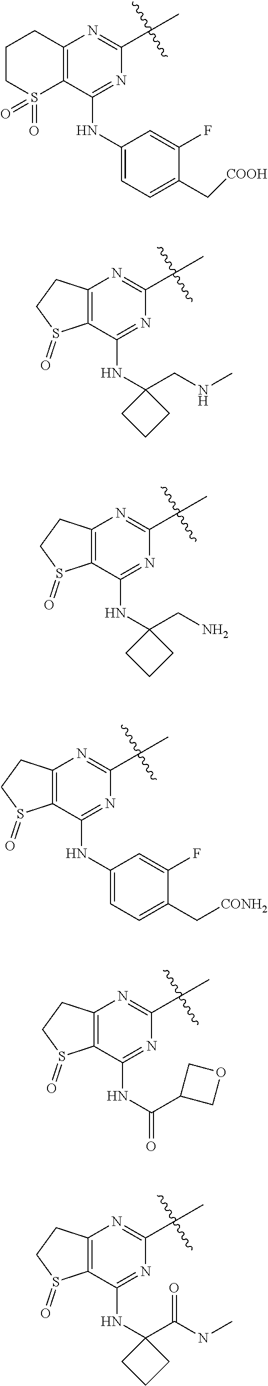 [SMILES: *c1nc2c(c(Nc3ccc(CC(=O)O)c(F)c3)n1)S(=O)(=O)CCC2 ; *c1nc2c(c(NC3(CNC)CCC3)n1)S(=O)CC2 ; *c1nc2c(c(NC3(CN)CCC3)n1)S(=O)CC2 ; *c1nc2c(c(Nc3ccc(CC(N)=O)c(F)c3)n1)S(=O)CC2 ; *c1nc2c(c(NC(=O)C3COC3)n1)S(=O)CC2 ; *c1nc2c(c(NC3(C(=O)NC)CCC3)n1)S(=O)CC2]

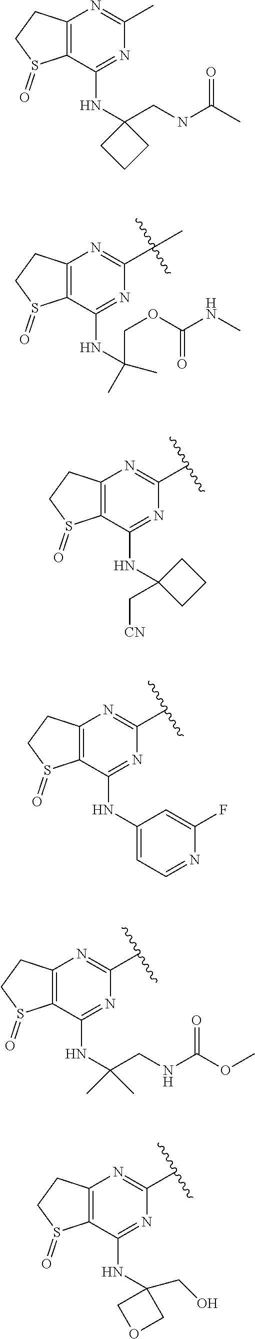 [SMILES: CC(=O)NCC1(Nc2nc(C)nc3c2S(=O)CC3)CCC1 ; *c1nc2c(c(NC(C)(C)COC(=O)NC)n1)S(=O)CC2 ; *c1nc2c(c(NC3(CC#N)CCC3)n1)S(=O)CC2 ; *c1nc2c(c(Nc3ccnc(F)c3)n1)S(=O)CC2 ; *c1nc2c(c(NC(C)(C)CNC(=O)OC)n1)S(=O)CC2 ; *c1nc2c(c(NC3(CO)COC3)n1)S(=O)CC2]

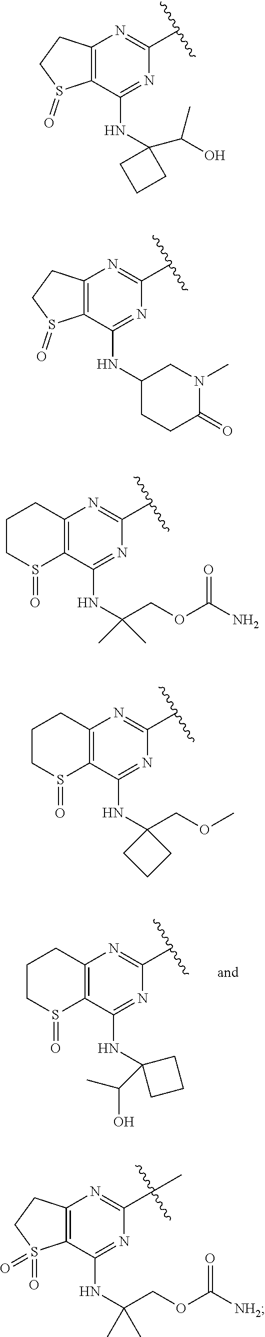 [SMILES: *c1nc2c(c(NC3(C(C)O)CCC3)n1)S(=O)CC2 ; *c1nc2c(c(NC3CCC(=O)N(C)C3)n1)S(=O)CC2 ; *c1nc2c(c(NC(C)(C)COC(N)=O)n1)S(=O)CCC2 ; *c1nc2c(c(NC3(COC)CCC3)n1)S(=O)CCC2 ; *c1nc2c(c(NC3(C(C)O)CCC3)n1)S(=O)CCC2 ; *c1nc2c(c(NC(C)(C)COC(N)=O)n1)S(=O)(=O)CC2]

-(L₁)n-(L₂)m- is selected from:

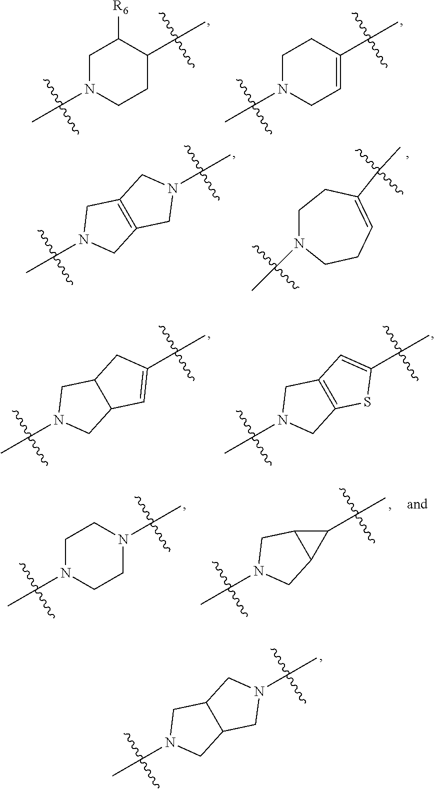

with R₆ being H;
-L₄- is selected from:

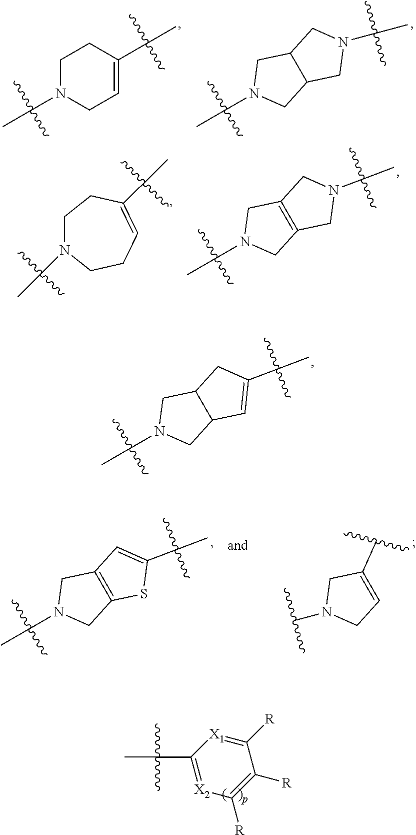

is selected from one of the following structures optionally substituted with 1-3 R_{R}:

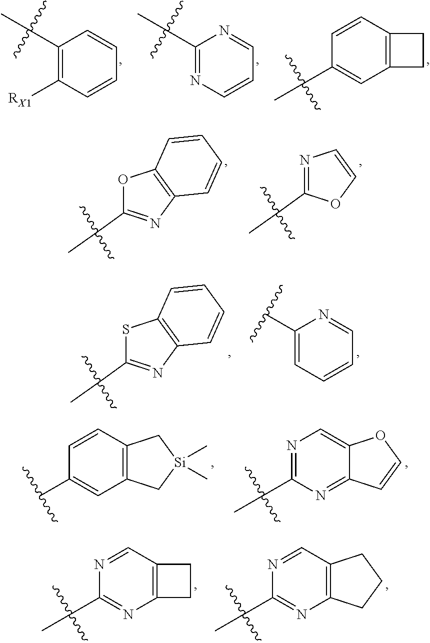

and each R_{R} is independently selected from F, Cl, Br, methyl, isopropyl, ethoxy, methoxy, ethynyl, propynyl, cyclopropyl, cyclobutyl, dimethylamino, and

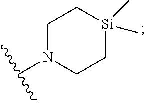 [SMILES: *N1CC[Si](C)(C)CC1]

and R_{X1} is H;
or R_{X1} and R₆, together with the atom to which they are attached, form morpholinyl, furyl, pyrrolyl, or thienyl.

Solution 20 of the present disclosure relates to a compound selected from one of the structures in Table 1, or stereoisomers or pharmaceutically acceptable salts thereof:

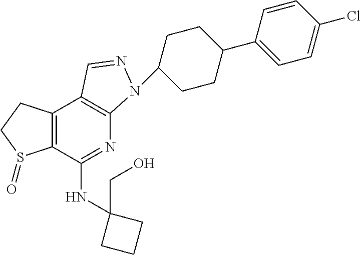 [SMILES: O=S1CCc2c1c(NC1(CO)CCC1)nc1c2cnn1C1CCC(c2ccc(Cl)cc2)CC1]

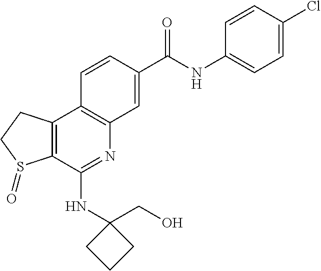 [SMILES: O=C(Nc1ccc(Cl)cc1)c1ccc2c3c(c(NC4(CO)CCC4)nc2c1)S(=O)CC3]

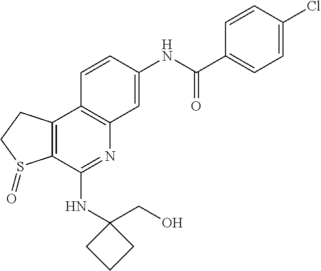 [SMILES: O=C(Nc1ccc2c3c(c(NC4(CO)CCC4)nc2c1)S(=O)CC3)c1ccc(Cl)cc1]

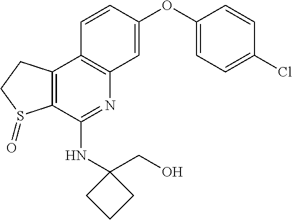 [SMILES: O=S1CCc2c1c(NC1(CO)CCC1)nc1cc(Oc3ccc(Cl)cc3)ccc21]

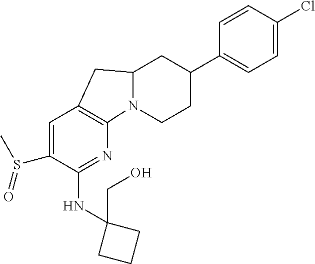 [SMILES: CS(=O)c1cc2c(nc1NC1(CO)CCC1)N1CCC(c3ccc(Cl)cc3)CC1C2]

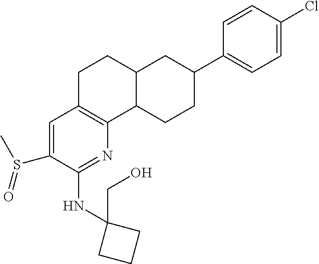 [SMILES: CS(=O)c1cc2c(nc1NC1(CO)CCC1)C1CCC(c3ccc(Cl)cc3)CC1CC2]

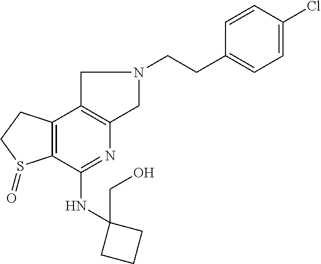 [SMILES: O=S1CCc2c3c(nc(NC4(CO)CCC4)c21)CN(CCc1ccc(Cl)cc1)C3]

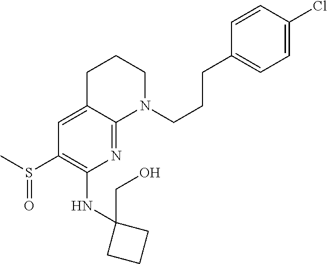 [SMILES: CS(=O)c1cc2c(nc1NC1(CO)CCC1)N(CCCc1ccc(Cl)cc1)CCC2]

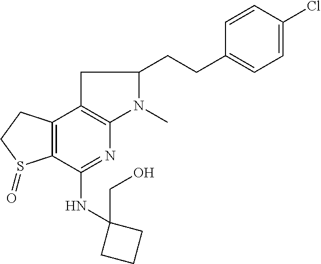 [SMILES: CN1c2nc(NC3(CO)CCC3)c3c(c2CC1CCc1ccc(Cl)cc1)CCS3=O]

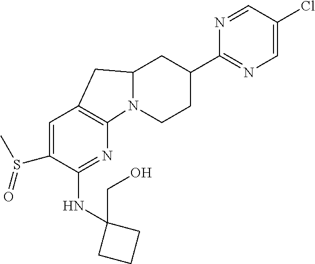 [SMILES: CS(=O)c1cc2c(nc1NC1(CO)CCC1)N1CCC(c3ncc(Cl)cn3)CC1C2]

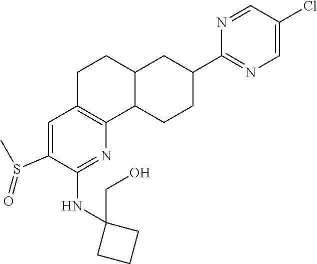 [SMILES: CS(=O)c1cc2c(nc1NC1(CO)CCC1)C1CCC(c3ncc(Cl)cn3)CC1CC2]

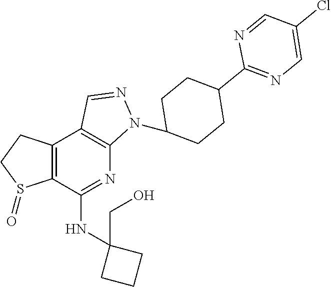 [SMILES: O=S1CCc2c1c(NC1(CO)CCC1)nc1c2cnn1C1CCC(c2ncc(Cl)cn2)CC1]

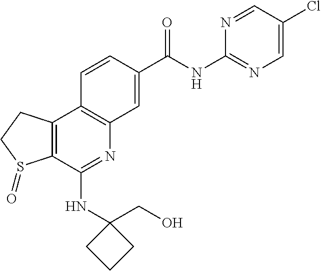 [SMILES: O=C(Nc1ncc(Cl)cn1)c1ccc2c3c(c(NC4(CO)CCC4)nc2c1)S(=O)CC3]

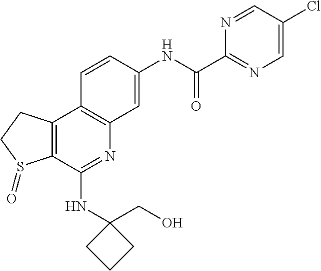 [SMILES: O=C(Nc1ccc2c3c(c(NC4(CO)CCC4)nc2c1)S(=O)CC3)c1ncc(Cl)cn1]

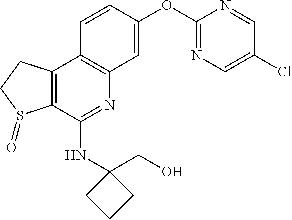 [SMILES: O=S1CCc2c1c(NC1(CO)CCC1)nc1cc(Oc3ncc(Cl)cn3)ccc21]

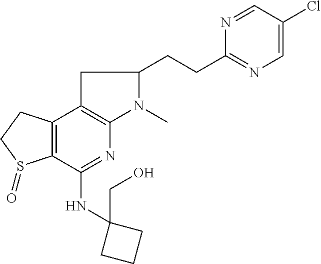 [SMILES: CN1c2nc(NC3(CO)CCC3)c3c(c2CC1CCc1ncc(Cl)cn1)CCS3=O]

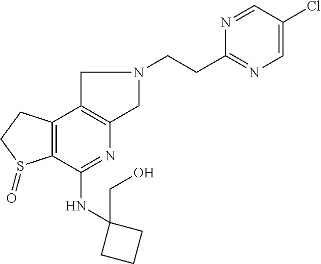 [SMILES: O=S1CCc2c3c(nc(NC4(CO)CCC4)c21)CN(CCc1ncc(Cl)cn1)C3]

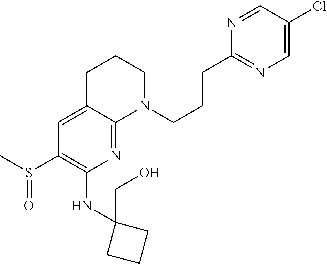 [SMILES: CS(=O)c1cc2c(nc1NC1(CO)CCC1)N(CCCc1ncc(Cl)cn1)CCC2]

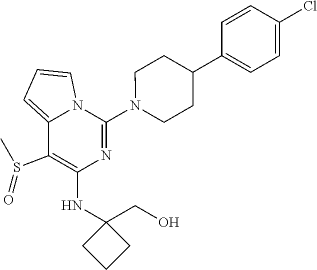 [SMILES: CS(=O)c1c(NC2(CO)CCC2)nc(N2CCC(c3ccc(Cl)cc3)CC2)n2cccc12]

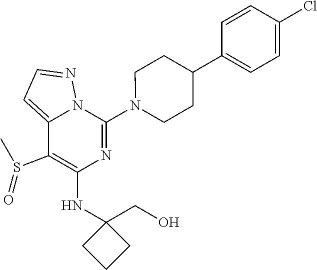 [SMILES: CS(=O)c1c(NC2(CO)CCC2)nc(N2CCC(c3ccc(Cl)cc3)CC2)n2nccc12]

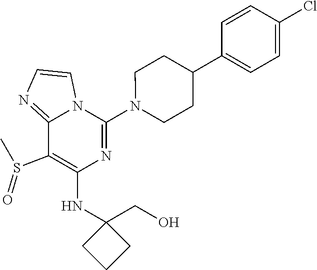 [SMILES: CS(=O)c1c(NC2(CO)CCC2)nc(N2CCC(c3ccc(Cl)cc3)CC2)n2ccnc12]

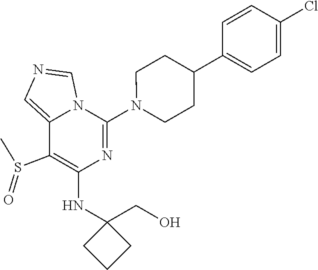 [SMILES: CS(=O)c1c(NC2(CO)CCC2)nc(N2CCC(c3ccc(Cl)cc3)CC2)n2cncc12]

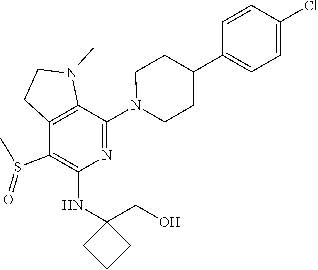 [SMILES: CN1CCc2c1c(N1CCC(c3ccc(Cl)cc3)CC1)nc(NC1(CO)CCC1)c2S(C)=O]

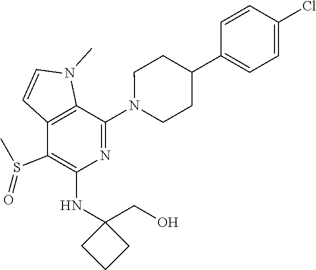 [SMILES: Cn1ccc2c(S(C)=O)c(NC3(CO)CCC3)nc(N3CCC(c4ccc(Cl)cc4)CC3)c21]

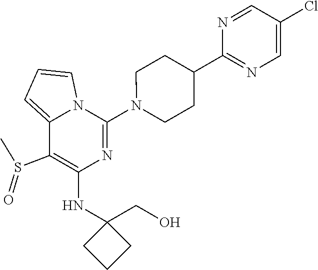 [SMILES: CS(=O)c1c(NC2(CO)CCC2)nc(N2CCC(c3ncc(Cl)cn3)CC2)n2cccc12]

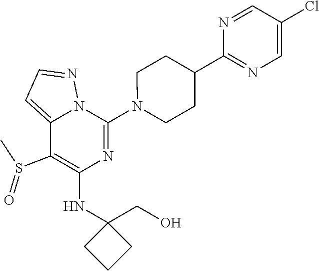 [SMILES: CS(=O)c1c(NC2(CO)CCC2)nc(N2CCC(c3ncc(Cl)cn3)CC2)n2nccc12]

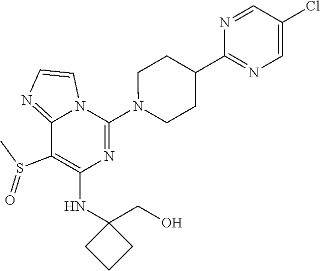 [SMILES: CS(=O)c1c(NC2(CO)CCC2)nc(N2CCC(c3ncc(Cl)cn3)CC2)n2ccnc12]

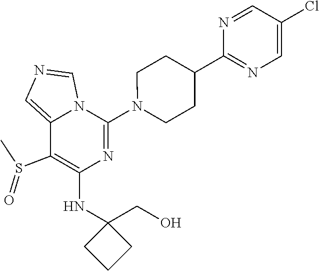 [SMILES: CS(=O)c1c(NC2(CO)CCC2)nc(N2CCC(c3ncc(Cl)cn3)CC2)n2cncc12]

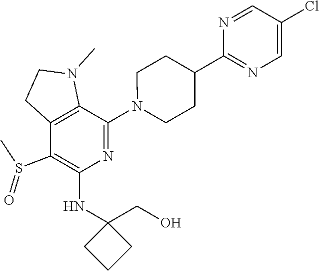 [SMILES: CN1CCc2c1c(N1CCC(c3ncc(Cl)cn3)CC1)nc(NC1(CO)CCC1)c2S(C)=O]

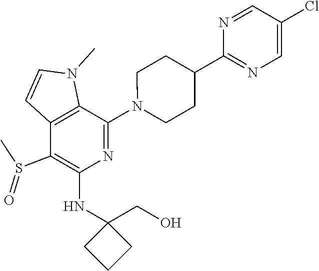 [SMILES: Cn1ccc2c(S(C)=O)c(NC3(CO)CCC3)nc(N3CCC(c4ncc(Cl)cn4)CC3)c21]

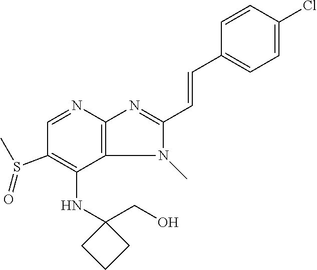 [SMILES: Cn1c(/C=C/c2ccc(Cl)cc2)nc2ncc(S(C)=O)c(NC3(CO)CCC3)c21]

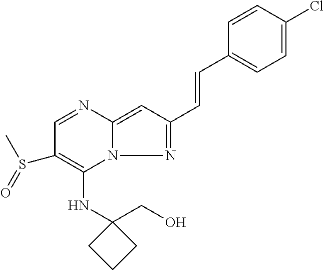 [SMILES: CS(=O)c1cnc2cc(/C=C/c3ccc(Cl)cc3)nn2c1NC1(CO)CCC1]

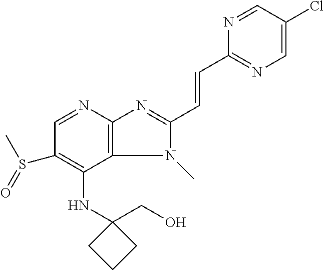 [SMILES: Cn1c(/C=C/c2ncc(Cl)cn2)nc2ncc(S(C)=O)c(NC3(CO)CCC3)c21]

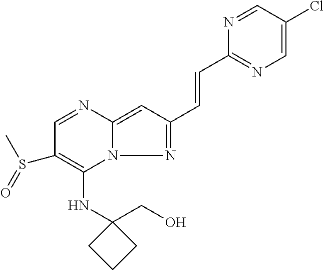 [SMILES: CS(=O)c1cnc2cc(/C=C/c3ncc(Cl)cn3)nn2c1NC1(CO)CCC1]

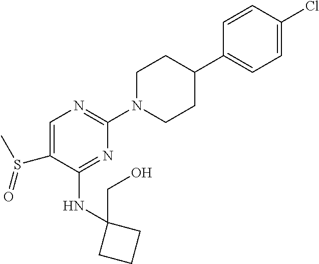 [SMILES: CS(=O)c1cnc(N2CCC(c3ccc(Cl)cc3)CC2)nc1NC1(CO)CCC1]

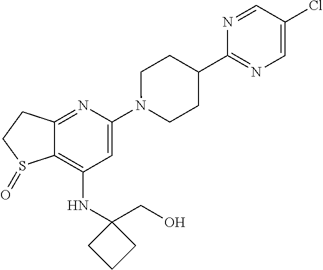 [SMILES: O=S1CCc2nc(N3CCC(c4ncc(Cl)cn4)CC3)cc(NC3(CO)CCC3)c21]

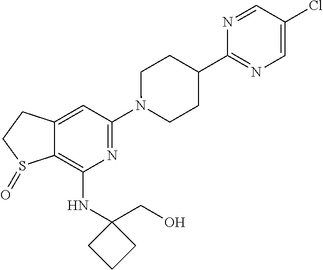 [SMILES: O=S1CCc2cc(N3CCC(c4ncc(Cl)cn4)CC3)nc(NC3(CO)CCC3)c21]

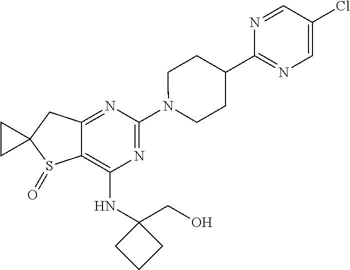 [SMILES: O=S1c2c(nc(N3CCC(c4ncc(Cl)cn4)CC3)nc2NC2(CO)CCC2)CC12CC2]

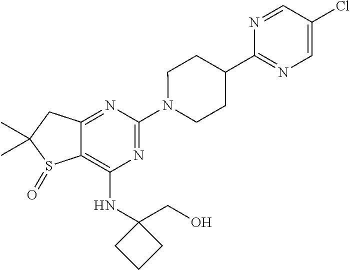 [SMILES: CC1(C)Cc2nc(N3CCC(c4ncc(Cl)cn4)CC3)nc(NC3(CO)CCC3)c2S1=O]

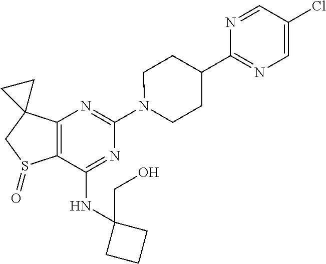 [SMILES: O=S1CC2(CC2)c2nc(N3CCC(c4ncc(Cl)cn4)CC3)nc(NC3(CO)CCC3)c21]

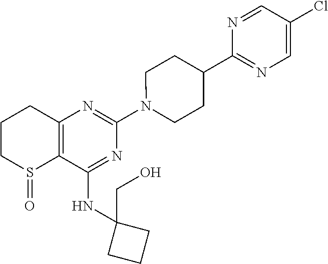 [SMILES: O=S1CCCc2nc(N3CCC(c4ncc(Cl)cn4)CC3)nc(NC3(CO)CCC3)c21]

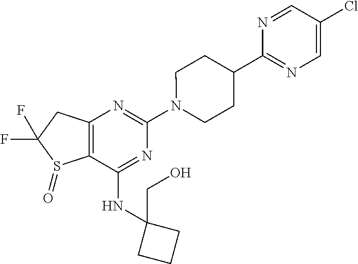 [SMILES: O=S1c2c(nc(N3CCC(c4ncc(Cl)cn4)CC3)nc2NC2(CO)CCC2)CC1(F)F]

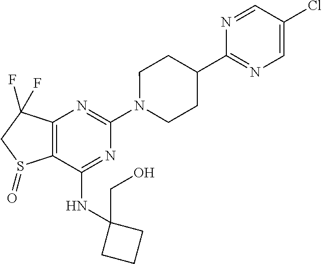 [SMILES: O=S1CC(F)(F)c2nc(N3CCC(c4ncc(Cl)cn4)CC3)nc(NC3(CO)CCC3)c21]

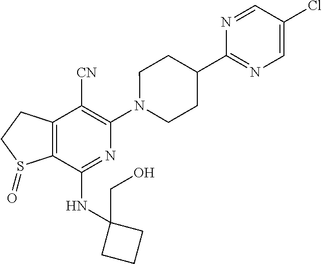 [SMILES: N#Cc1c(N2CCC(c3ncc(Cl)cn3)CC2)nc(NC2(CO)CCC2)c2c1CCS2=O]

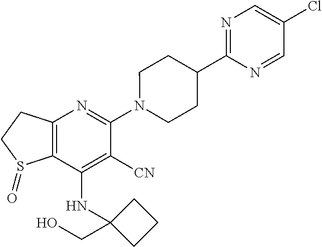 [SMILES: N#Cc1c(N2CCC(c3ncc(Cl)cn3)CC2)nc2c(c1NC1(CO)CCC1)S(=O)CC2]

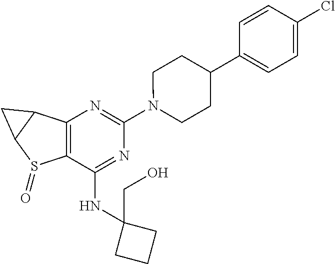 [SMILES: O=S1c2c(NC3(CO)CCC3)nc(N3CCC(c4ccc(Cl)cc4)CC3)nc2C2CC21]

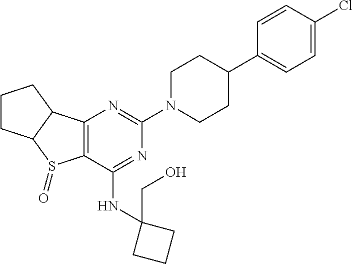 [SMILES: O=S1c2c(NC3(CO)CCC3)nc(N3CCC(c4ccc(Cl)cc4)CC3)nc2C2CCCC21]

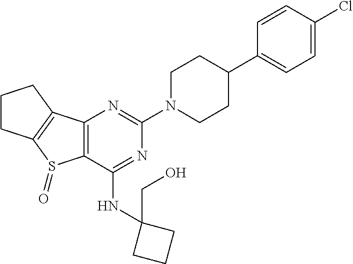 [SMILES: O=S1C2=C(CCC2)c2nc(N3CCC(c4ccc(Cl)cc4)CC3)nc(NC3(CO)CCC3)c21]

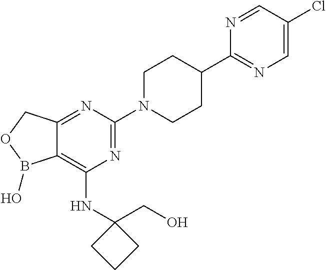 [SMILES: OCC1(Nc2nc(N3CCC(c4ncc(Cl)cn4)CC3)nc3c2B(O)OC3)CCC1]

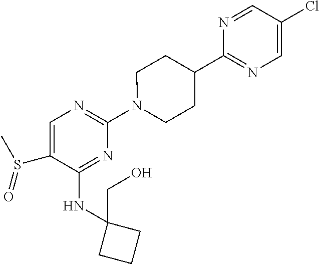 [SMILES: CS(=O)c1cnc(N2CCC(c3ncc(Cl)cn3)CC2)nc1NC1(CO)CCC1]

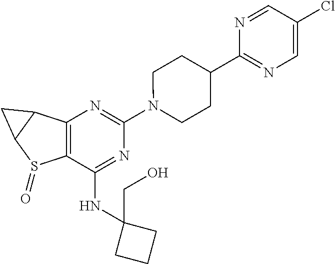 [SMILES: O=S1c2c(NC3(CO)CCC3)nc(N3CCC(c4ncc(Cl)cn4)CC3)nc2C2CC21]

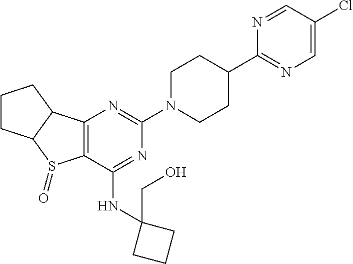 [SMILES: O=S1c2c(NC3(CO)CCC3)nc(N3CCC(c4ncc(Cl)cn4)CC3)nc2C2CCCC21]

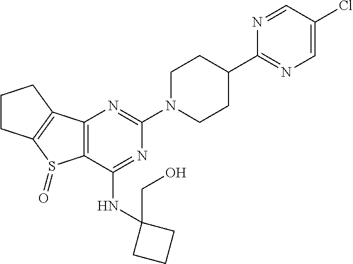 [SMILES: O=S1C2=C(CCC2)c2nc(N3CCC(c4ncc(Cl)cn4)CC3)nc(NC3(CO)CCC3)c21]

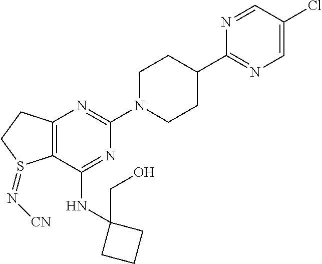 [SMILES: N#C/N=S1/CCc2nc(N3CCC(c4ncc(Cl)cn4)CC3)nc(NC3(CO)CCC3)c21]

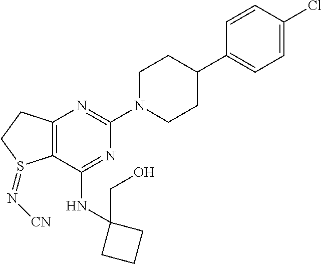 [SMILES: N#C/N=S1/CCc2nc(N3CCC(c4ccc(Cl)cc4)CC3)nc(NC3(CO)CCC3)c21]

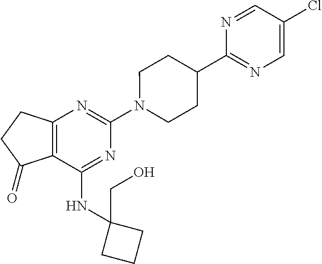 [SMILES: O=C1CCc2nc(N3CCC(c4ncc(Cl)cn4)CC3)nc(NC3(CO)CCC3)c21]

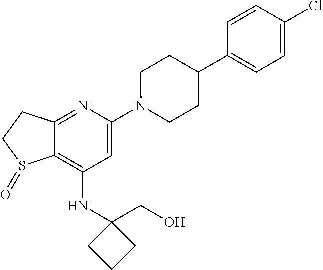 [SMILES: O=S1CCc2nc(N3CCC(c4ccc(Cl)cc4)CC3)cc(NC3(CO)CCC3)c21]

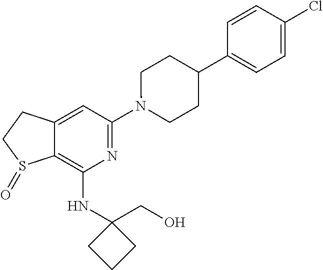 [SMILES: O=S1CCc2cc(N3CCC(c4ccc(Cl)cc4)CC3)nc(NC3(CO)CCC3)c21]

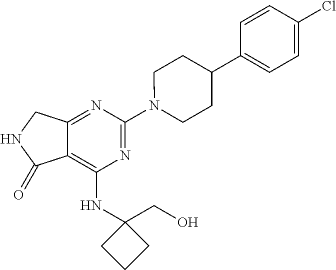 [SMILES: O=C1NCc2nc(N3CCC(c4ccc(Cl)cc4)CC3)nc(NC3(CO)CCC3)c21]

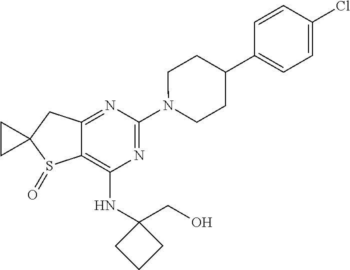 [SMILES: O=S1c2c(nc(N3CCC(c4ccc(Cl)cc4)CC3)nc2NC2(CO)CCC2)CC12CC2]

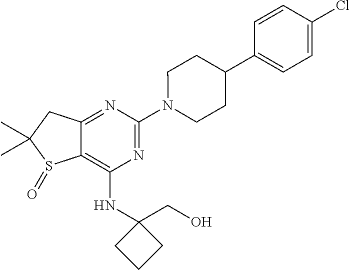 [SMILES: CC1(C)Cc2nc(N3CCC(c4ccc(Cl)cc4)CC3)nc(NC3(CO)CCC3)c2S1=O]

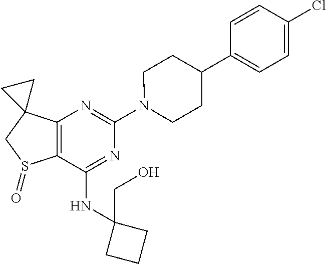 [SMILES: O=S1CC2(CC2)c2nc(N3CCC(c4ccc(Cl)cc4)CC3)nc(NC3(CO)CCC3)c21]

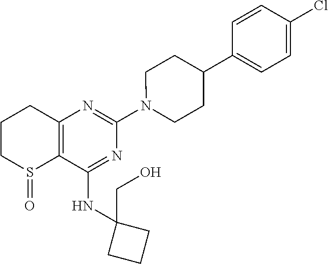 [SMILES: O=S1CCCc2nc(N3CCC(c4ccc(Cl)cc4)CC3)nc(NC3(CO)CCC3)c21]

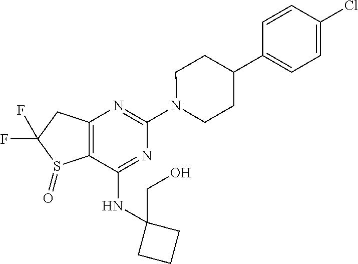 [SMILES: O=S1c2c(nc(N3CCC(c4ccc(Cl)cc4)CC3)nc2NC2(CO)CCC2)CC1(F)F]

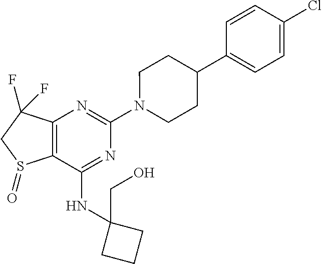 [SMILES: O=S1CC(F)(F)c2nc(N3CCC(c4ccc(Cl)cc4)CC3)nc(NC3(CO)CCC3)c21]

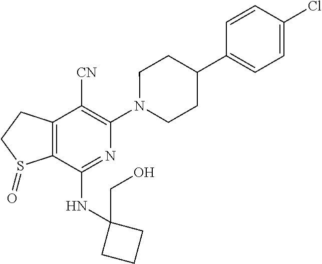 [SMILES: N#Cc1c(N2CCC(c3ccc(Cl)cc3)CC2)nc(NC2(CO)CCC2)c2c1CCS2=O]

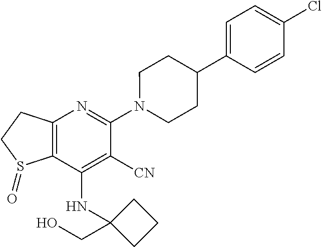 [SMILES: N#Cc1c(N2CCC(c3ccc(Cl)cc3)CC2)nc2c(c1NC1(CO)CCC1)S(=O)CC2]

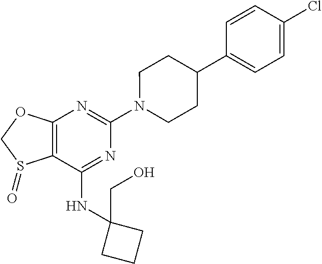 [SMILES: O=S1COc2nc(N3CCC(c4ccc(Cl)cc4)CC3)nc(NC3(CO)CCC3)c21]

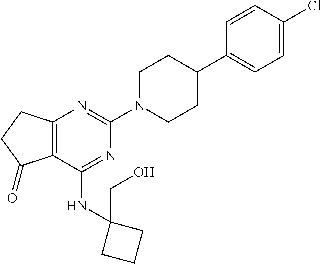 [SMILES: O=C1CCc2nc(N3CCC(c4ccc(Cl)cc4)CC3)nc(NC3(CO)CCC3)c21]

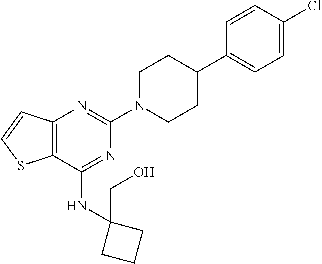 [SMILES: OCC1(Nc2nc(N3CCC(c4ccc(Cl)cc4)CC3)nc3ccsc23)CCC1]

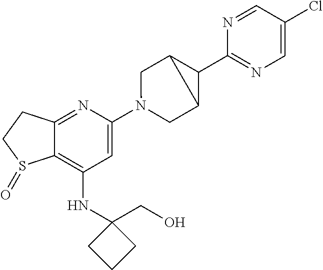 [SMILES: O=S1CCc2nc(N3CC4C(C3)C4c3ncc(Cl)cn3)cc(NC3(CO)CCC3)c21]

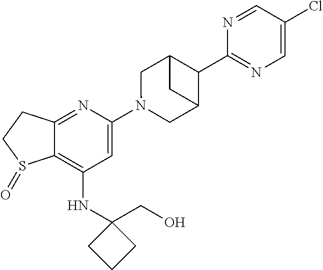 [SMILES: O=S1CCc2nc(N3CC4CC(C3)C4c3ncc(Cl)cn3)cc(NC3(CO)CCC3)c21]

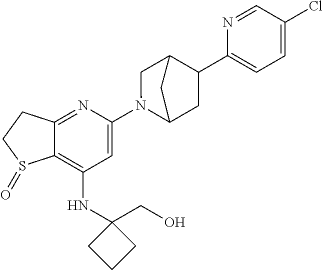 [SMILES: O=S1CCc2nc(N3CC4CC3CC4c3ccc(Cl)cn3)cc(NC3(CO)CCC3)c21]

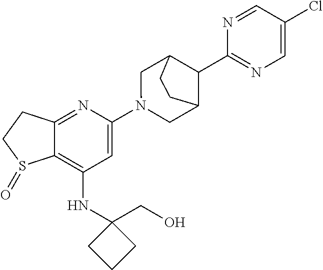 [SMILES: O=S1CCc2nc(N3CC4CCC(C3)C4c3ncc(Cl)cn3)cc(NC3(CO)CCC3)c21]

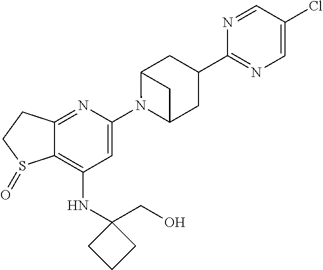 [SMILES: O=S1CCc2nc(N3C4CC(c5ncc(Cl)cn5)CC3C4)cc(NC3(CO)CCC3)c21]

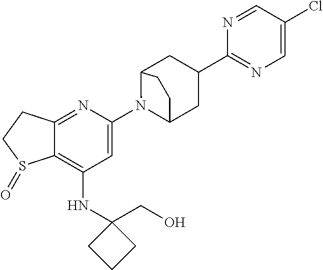 [SMILES: O=S1CCc2nc(N3C4CCC3CC(c3ncc(Cl)cn3)C4)cc(NC3(CO)CCC3)c21]

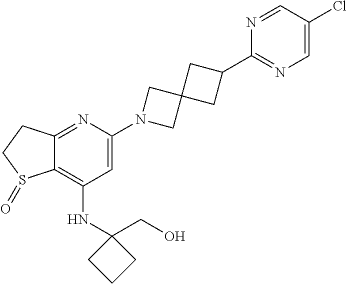 [SMILES: O=S1CCc2nc(N3CC4(CC(c5ncc(Cl)cn5)C4)C3)cc(NC3(CO)CCC3)c21]

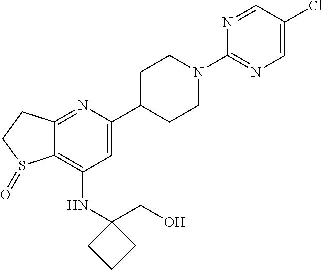 [SMILES: O=S1CCc2nc(C3CCN(c4ncc(Cl)cn4)CC3)cc(NC3(CO)CCC3)c21]

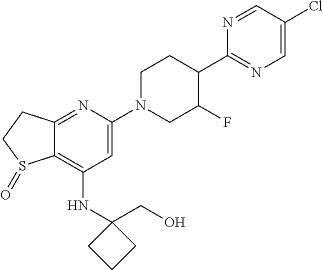 [SMILES: O=S1CCc2nc(N3CCC(c4ncc(Cl)cn4)C(F)C3)cc(NC3(CO)CCC3)c21]

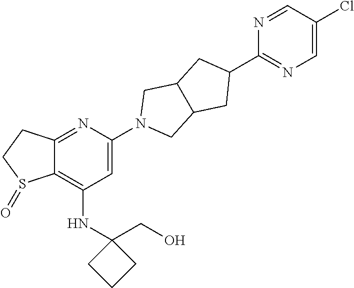 [SMILES: O=S1CCc2nc(N3CC4CC(c5ncc(Cl)cn5)CC4C3)cc(NC3(CO)CCC3)c21]

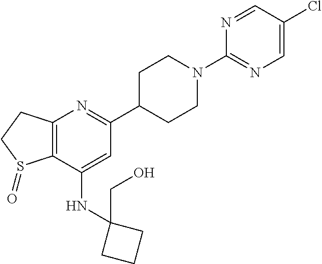 [SMILES: O=S1CCc2nc(C3CCN(c4ncc(Cl)cn4)CC3)cc(NC3(CO)CCC3)c21]

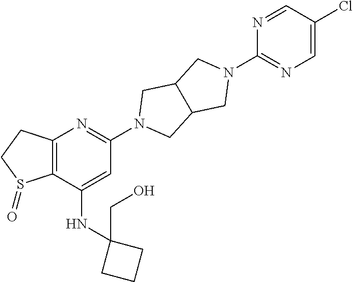 [SMILES: O=S1CCc2nc(N3CC4CN(c5ncc(Cl)cn5)CC4C3)cc(NC3(CO)CCC3)c21]

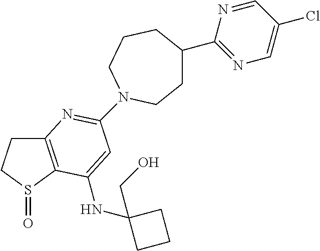 [SMILES: O=S1CCc2nc(N3CCCC(c4ncc(Cl)cn4)CC3)cc(NC3(CO)CCC3)c21]

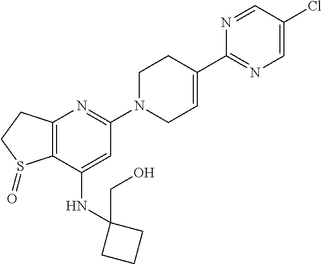 [SMILES: O=S1CCc2nc(N3CC=C(c4ncc(Cl)cn4)CC3)cc(NC3(CO)CCC3)c21]

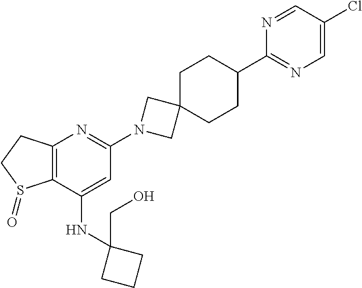 [SMILES: O=S1CCc2nc(N3CC4(CCC(c5ncc(Cl)cn5)CC4)C3)cc(NC3(CO)CCC3)c21]

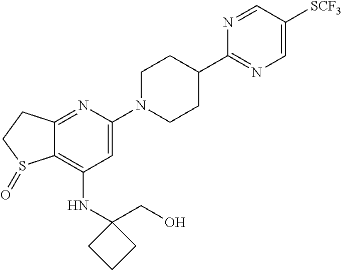

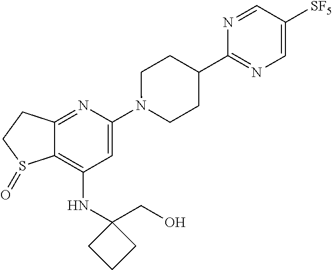

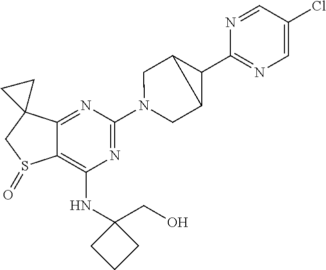 [SMILES: O=S1CC2(CC2)c2nc(N3CC4C(C3)C4c3ncc(Cl)cn3)nc(NC3(CO)CCC3)c21]

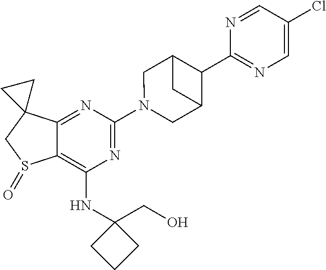 [SMILES: O=S1CC2(CC2)c2nc(N3CC4CC(C3)C4c3ncc(Cl)cn3)nc(NC3(CO)CCC3)c21]

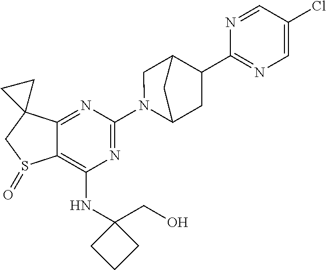 [SMILES: O=S1CC2(CC2)c2nc(N3CC4CC3CC4c3ncc(Cl)cn3)nc(NC3(CO)CCC3)c21]

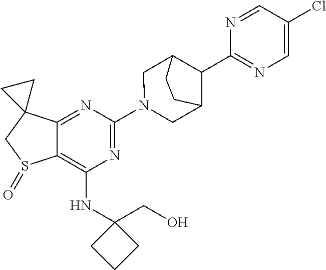 [SMILES: O=S1CC2(CC2)c2nc(N3CC4CCC(C3)C4c3ncc(Cl)cn3)nc(NC3(CO)CCC3)c21]

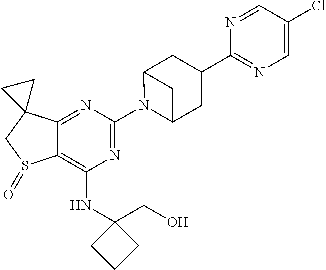 [SMILES: O=S1CC2(CC2)c2nc(N3C4CC(c5ncc(Cl)cn5)CC3C4)nc(NC3(CO)CCC3)c21]

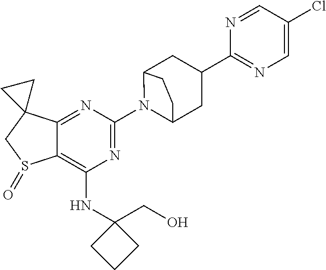 [SMILES: O=S1CC2(CC2)c2nc(N3C4CCC3CC(c3ncc(Cl)cn3)C4)nc(NC3(CO)CCC3)c21]

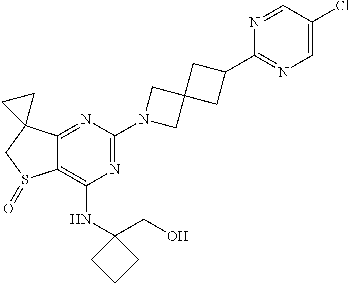 [SMILES: O=S1CC2(CC2)c2nc(N3CC4(CC(c5ncc(Cl)cn5)C4)C3)nc(NC3(CO)CCC3)c21]

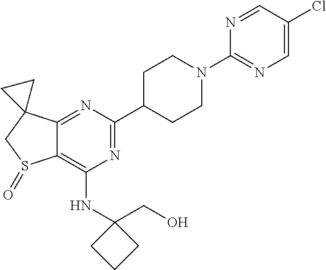 [SMILES: O=S1CC2(CC2)c2nc(C3CCN(c4ncc(Cl)cn4)CC3)nc(NC3(CO)CCC3)c21]

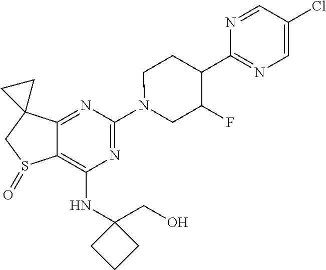 [SMILES: O=S1CC2(CC2)c2nc(N3CCC(c4ncc(Cl)cn4)C(F)C3)nc(NC3(CO)CCC3)c21]

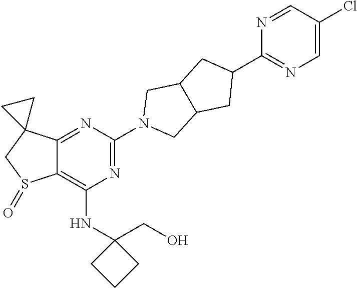 [SMILES: O=S1CC2(CC2)c2nc(N3CC4CC(c5ncc(Cl)cn5)CC4C3)nc(NC3(CO)CCC3)c21]

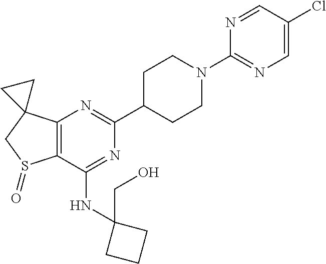 [SMILES: O=S1CC2(CC2)c2nc(C3CCN(c4ncc(Cl)cn4)CC3)nc(NC3(CO)CCC3)c21]

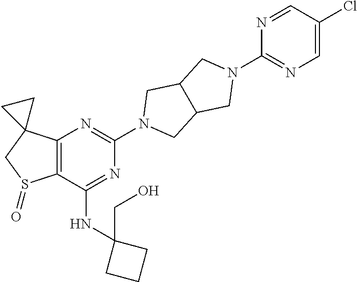 [SMILES: O=S1CC2(CC2)c2nc(N3CC4CN(c5ncc(Cl)cn5)CC4C3)nc(NC3(CO)CCC3)c21]

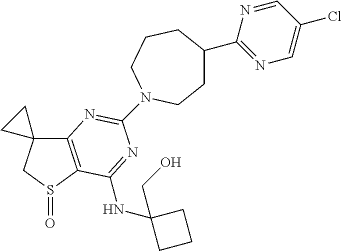 [SMILES: O=S1CC2(CC2)c2nc(N3CCCC(c4ncc(Cl)cn4)CC3)nc(NC3(CO)CCC3)c21]

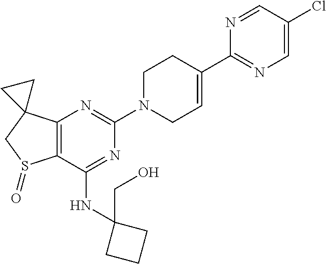 [SMILES: O=S1CC2(CC2)c2nc(N3CC=C(c4ncc(Cl)cn4)CC3)nc(NC3(CO)CCC3)c21]

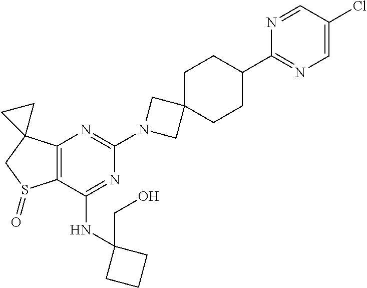 [SMILES: O=S1CC2(CC2)c2nc(N3CC4(CCC(c5ncc(Cl)cn5)CC4)C3)nc(NC3(CO)CCC3)c21]

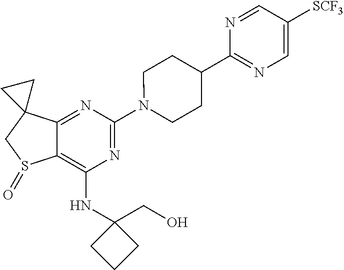

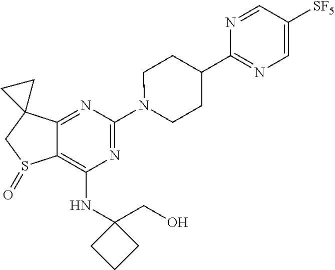

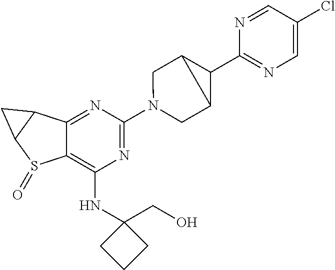 [SMILES: O=S1c2c(NC3(CO)CCC3)nc(N3CC4C(C3)C4c3ncc(Cl)cn3)nc2C2CC21]

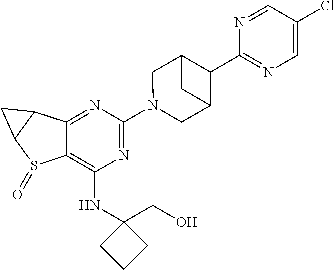 [SMILES: O=S1c2c(NC3(CO)CCC3)nc(N3CC4CC(C3)C4c3ncc(Cl)cn3)nc2C2CC21]

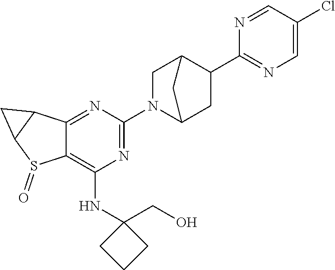 [SMILES: O=S1c2c(NC3(CO)CCC3)nc(N3CC4CC3CC4c3ncc(Cl)cn3)nc2C2CC21]

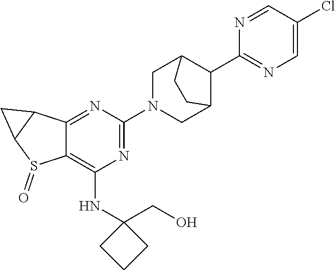 [SMILES: O=S1c2c(NC3(CO)CCC3)nc(N3CC4CCC(C3)C4c3ncc(Cl)cn3)nc2C2CC21]

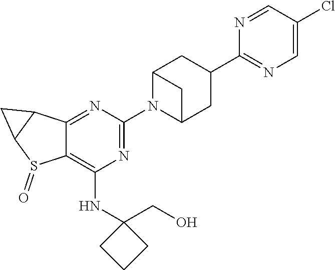 [SMILES: O=S1c2c(NC3(CO)CCC3)nc(N3C4CC(c5ncc(Cl)cn5)CC3C4)nc2C2CC21]

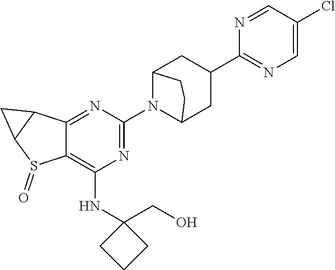 [SMILES: O=S1c2c(NC3(CO)CCC3)nc(N3C4CCC3CC(c3ncc(Cl)cn3)C4)nc2C2CC21]

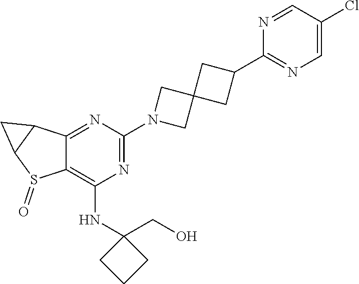 [SMILES: O=S1c2c(NC3(CO)CCC3)nc(N3CC4(CC(c5ncc(Cl)cn5)C4)C3)nc2C2CC21]

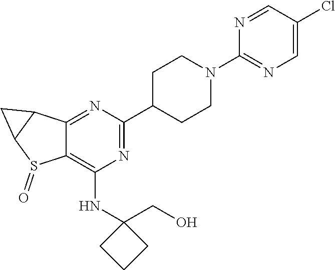 [SMILES: O=S1c2c(NC3(CO)CCC3)nc(C3CCN(c4ncc(Cl)cn4)CC3)nc2C2CC21]

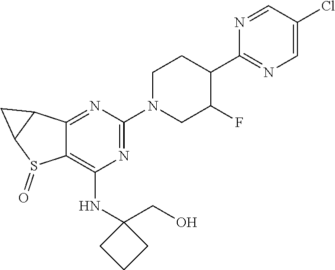 [SMILES: O=S1c2c(NC3(CO)CCC3)nc(N3CCC(c4ncc(Cl)cn4)C(F)C3)nc2C2CC21]

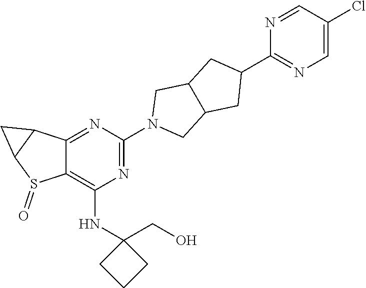 [SMILES: O=S1c2c(NC3(CO)CCC3)nc(N3CC4CC(c5ncc(Cl)cn5)CC4C3)nc2C2CC21]

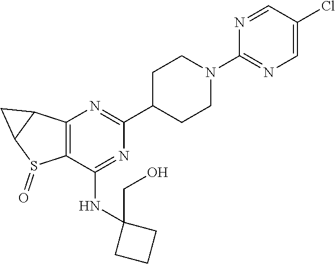 [SMILES: O=S1c2c(NC3(CO)CCC3)nc(C3CCN(c4ncc(Cl)cn4)CC3)nc2C2CC21]

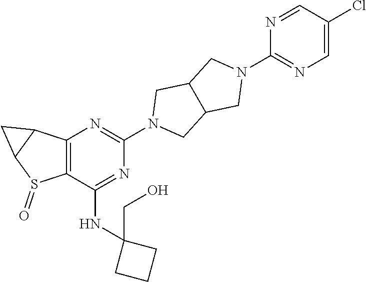 [SMILES: O=S1c2c(NC3(CO)CCC3)nc(N3CC4CN(c5ncc(Cl)cn5)CC4C3)nc2C2CC21]

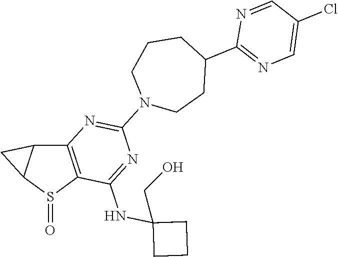 [SMILES: O=S1c2c(NC3(CO)CCC3)nc(N3CCCC(c4ncc(Cl)cn4)CC3)nc2C2CC21]

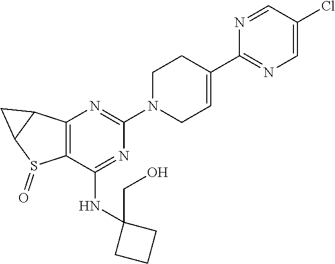 [SMILES: O=S1c2c(NC3(CO)CCC3)nc(N3CC=C(c4ncc(Cl)cn4)CC3)nc2C2CC21]

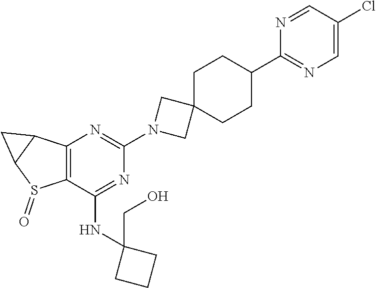 [SMILES: O=S1c2c(NC3(CO)CCC3)nc(N3CC4(CCC(c5ncc(Cl)cn5)CC4)C3)nc2C2CC21]

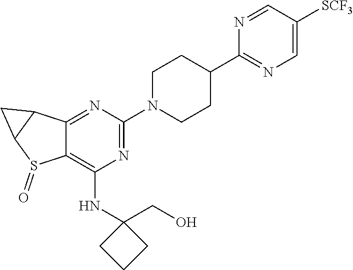

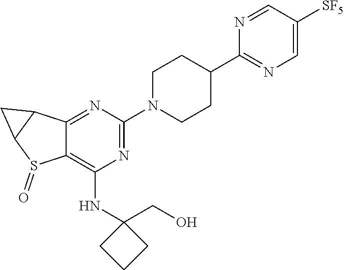

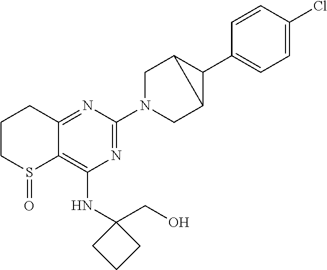 [SMILES: O=S1CCCc2nc(N3CC4C(C3)C4c3ccc(Cl)cc3)nc(NC3(CO)CCC3)c21]

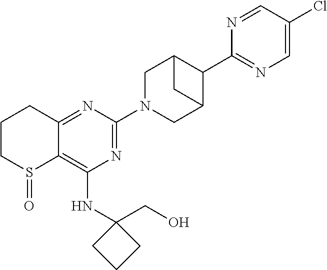 [SMILES: O=S1CCCc2nc(N3CC4CC(C3)C4c3ncc(Cl)cn3)nc(NC3(CO)CCC3)c21]

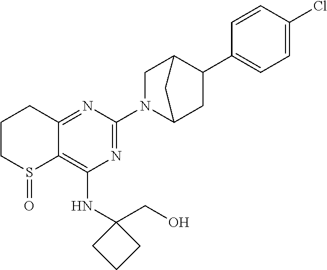 [SMILES: O=S1CCCc2nc(N3CC4CC3CC4c3ccc(Cl)cc3)nc(NC3(CO)CCC3)c21]

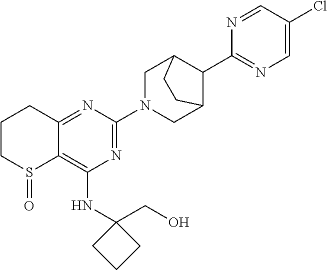 [SMILES: O=S1CCCc2nc(N3CC4CCC(C3)C4c3ncc(Cl)cn3)nc(NC3(CO)CCC3)c21]

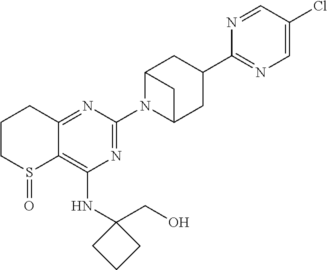 [SMILES: O=S1CCCc2nc(N3C4CC(c5ncc(Cl)cn5)CC3C4)nc(NC3(CO)CCC3)c21]

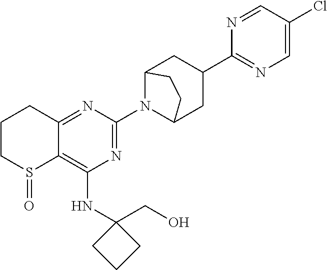 [SMILES: O=S1CCCc2nc(N3C4CCC3CC(c3ncc(Cl)cn3)C4)nc(NC3(CO)CCC3)c21]

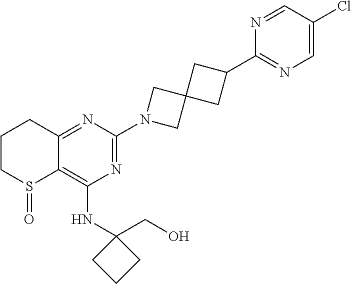 [SMILES: O=S1CCCc2nc(N3CC4(CC(c5ncc(Cl)cn5)C4)C3)nc(NC3(CO)CCC3)c21]

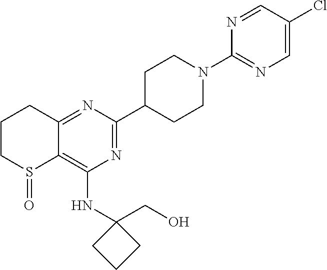 [SMILES: O=S1CCCc2nc(C3CCN(c4ncc(Cl)cn4)CC3)nc(NC3(CO)CCC3)c21]

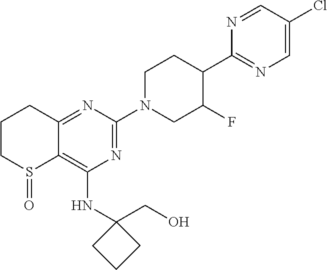 [SMILES: O=S1CCCc2nc(N3CCC(c4ncc(Cl)cn4)C(F)C3)nc(NC3(CO)CCC3)c21]

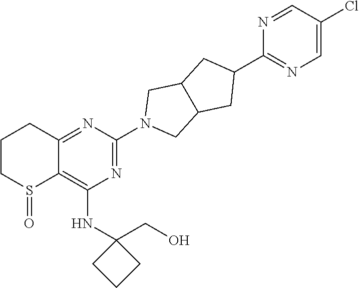 [SMILES: O=S1CCCc2nc(N3CC4CC(c5ncc(Cl)cn5)CC4C3)nc(NC3(CO)CCC3)c21]

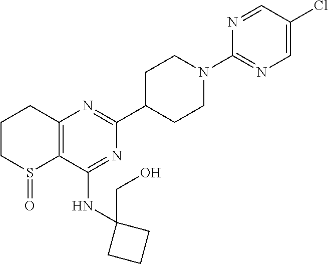 [SMILES: O=S1CCCc2nc(C3CCN(c4ncc(Cl)cn4)CC3)nc(NC3(CO)CCC3)c21]

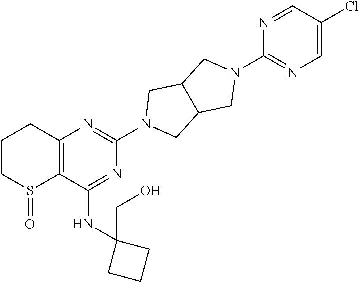 [SMILES: O=S1CCCc2nc(N3CC4CN(c5ncc(Cl)cn5)CC4C3)nc(NC3(CO)CCC3)c21]

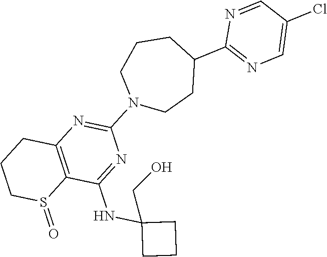 [SMILES: O=S1CCCc2nc(N3CCCC(c4ncc(Cl)cn4)CC3)nc(NC3(CO)CCC3)c21]

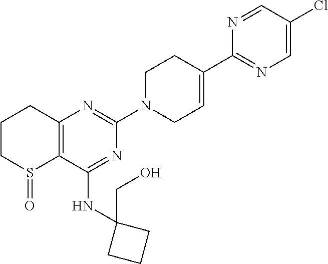 [SMILES: O=S1CCCc2nc(N3CC=C(c4ncc(Cl)cn4)CC3)nc(NC3(CO)CCC3)c21]

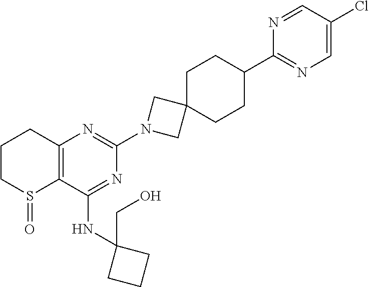 [SMILES: O=S1CCCc2nc(N3CC4(CCC(c5ncc(Cl)cn5)CC4)C3)nc(NC3(CO)CCC3)c21]

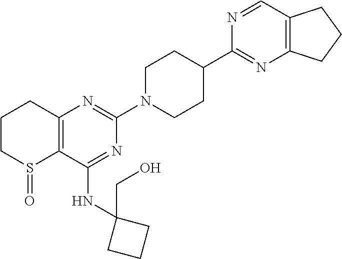 [SMILES: O=S1CCCc2nc(N3CCC(c4ncc5c(n4)CCC5)CC3)nc(NC3(CO)CCC3)c21]

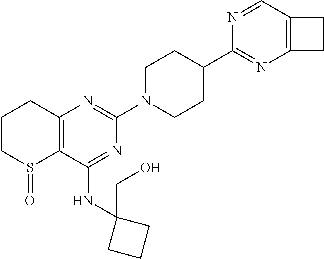 [SMILES: O=S1CCCc2nc(N3CCC(c4ncc5c(n4)CC5)CC3)nc(NC3(CO)CCC3)c21]

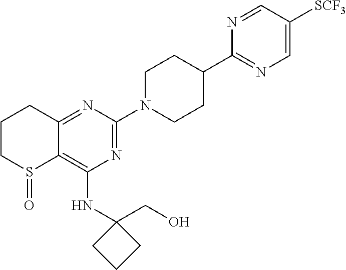

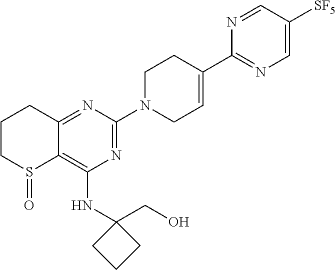

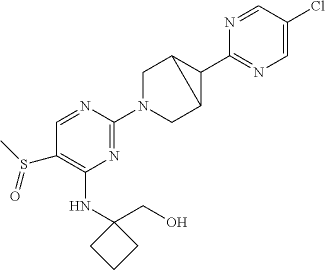 [SMILES: CS(=O)c1cnc(N2CC3C(C2)C3c2ncc(Cl)cn2)nc1NC1(CO)CCC1]

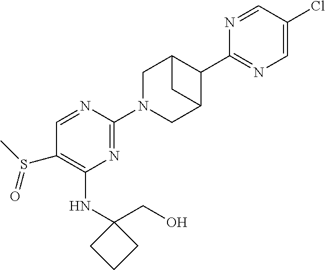 [SMILES: CS(=O)c1cnc(N2CC3CC(C2)C3c2ncc(Cl)cn2)nc1NC1(CO)CCC1]

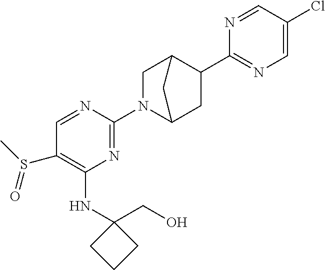 [SMILES: CS(=O)c1cnc(N2CC3CC2CC3c2ncc(Cl)cn2)nc1NC1(CO)CCC1]

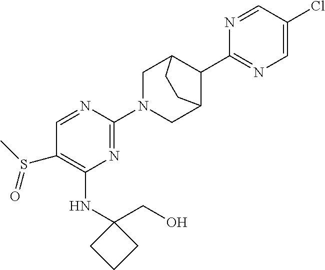 [SMILES: CS(=O)c1cnc(N2CC3CCC(C2)C3c2ncc(Cl)cn2)nc1NC1(CO)CCC1]

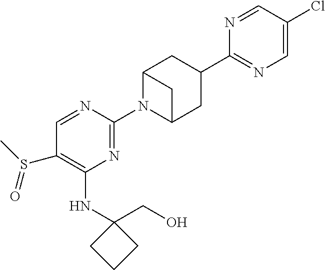 [SMILES: CS(=O)c1cnc(N2C3CC(c4ncc(Cl)cn4)CC2C3)nc1NC1(CO)CCC1]

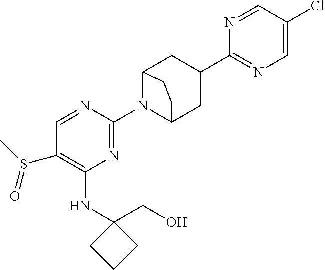 [SMILES: CS(=O)c1cnc(N2C3CCC2CC(c2ncc(Cl)cn2)C3)nc1NC1(CO)CCC1]

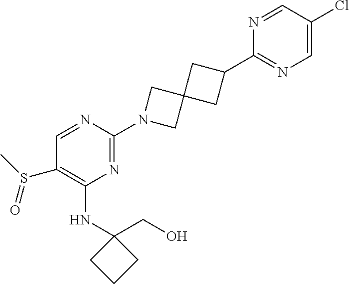 [SMILES: CS(=O)c1cnc(N2CC3(CC(c4ncc(Cl)cn4)C3)C2)nc1NC1(CO)CCC1]

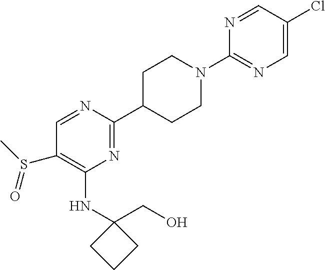 [SMILES: CS(=O)c1cnc(C2CCN(c3ncc(Cl)cn3)CC2)nc1NC1(CO)CCC1]

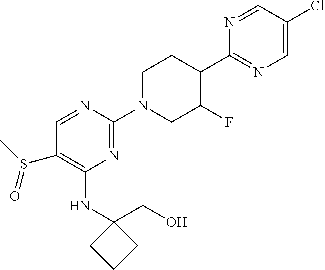 [SMILES: CS(=O)c1cnc(N2CCC(c3ncc(Cl)cn3)C(F)C2)nc1NC1(CO)CCC1]

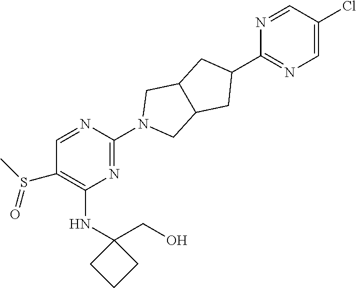 [SMILES: CS(=O)c1cnc(N2CC3CC(c4ncc(Cl)cn4)CC3C2)nc1NC1(CO)CCC1]

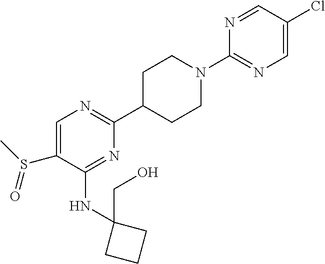 [SMILES: CS(=O)c1cnc(C2CCN(c3ncc(Cl)cn3)CC2)nc1NC1(CO)CCC1]

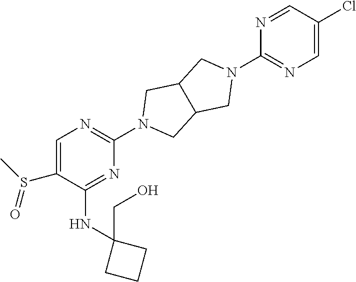 [SMILES: CS(=O)c1cnc(N2CC3CN(c4ncc(Cl)cn4)CC3C2)nc1NC1(CO)CCC1]

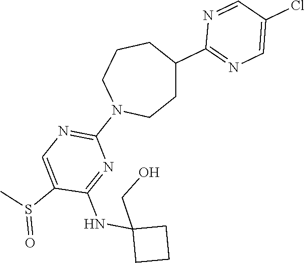 [SMILES: CS(=O)c1cnc(N2CCCC(c3ncc(Cl)cn3)CC2)nc1NC1(CO)CCC1]

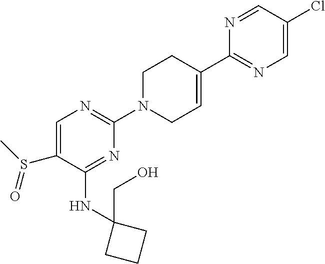 [SMILES: CS(=O)c1cnc(N2CC=C(c3ncc(Cl)cn3)CC2)nc1NC1(CO)CCC1]

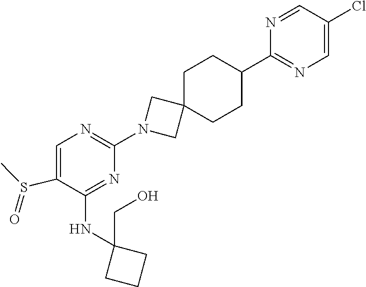 [SMILES: CS(=O)c1cnc(N2CC3(CCC(c4ncc(Cl)cn4)CC3)C2)nc1NC1(CO)CCC1]

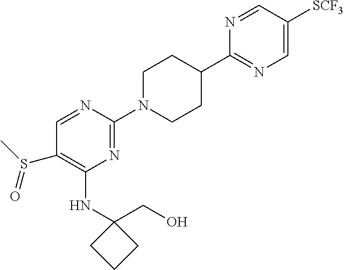

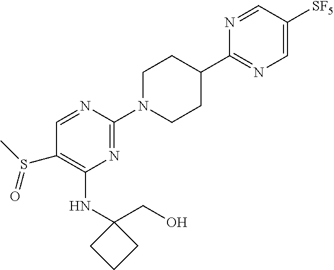

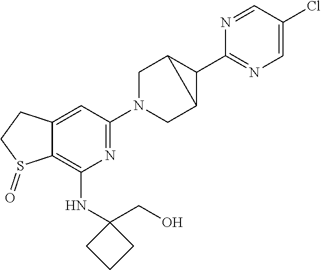 [SMILES: O=S1CCc2cc(N3CC4C(C3)C4c3ncc(Cl)cn3)nc(NC3(CO)CCC3)c21]

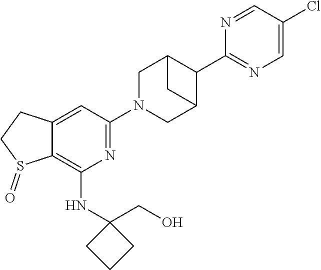 [SMILES: O=S1CCc2cc(N3CC4CC(C3)C4c3ncc(Cl)cn3)nc(NC3(CO)CCC3)c21]

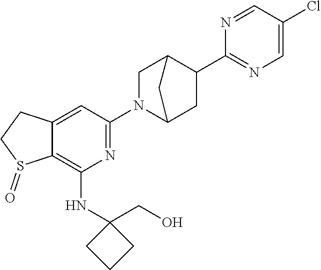 [SMILES: O=S1CCc2cc(N3CC4CC3CC4c3ncc(Cl)cn3)nc(NC3(CO)CCC3)c21]

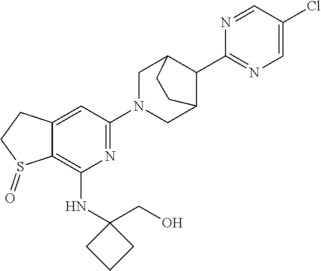 [SMILES: O=S1CCc2cc(N3CC4CCC(C3)C4c3ncc(Cl)cn3)nc(NC3(CO)CCC3)c21]

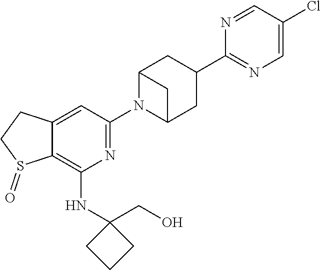 [SMILES: O=S1CCc2cc(N3C4CC(c5ncc(Cl)cn5)CC3C4)nc(NC3(CO)CCC3)c21]

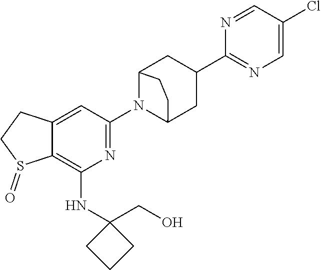 [SMILES: O=S1CCc2cc(N3C4CCC3CC(c3ncc(Cl)cn3)C4)nc(NC3(CO)CCC3)c21]

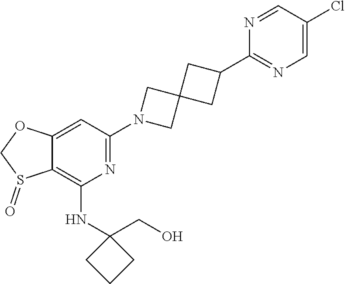 [SMILES: O=S1COc2cc(N3CC4(CC(c5ncc(Cl)cn5)C4)C3)nc(NC3(CO)CCC3)c21]

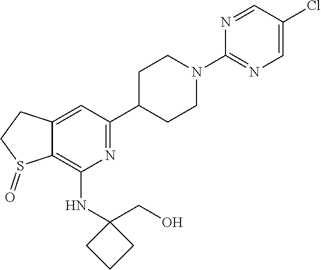 [SMILES: O=S1CCc2cc(C3CCN(c4ncc(Cl)cn4)CC3)nc(NC3(CO)CCC3)c21]

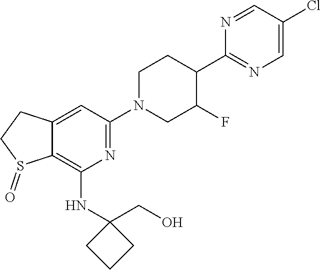 [SMILES: O=S1CCc2cc(N3CCC(c4ncc(Cl)cn4)C(F)C3)nc(NC3(CO)CCC3)c21]

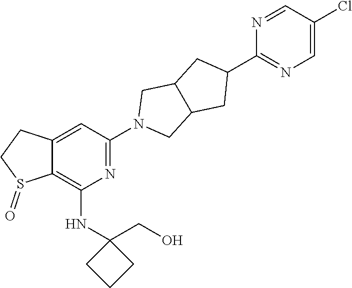 [SMILES: O=S1CCc2cc(N3CC4CC(c5ncc(Cl)cn5)CC4C3)nc(NC3(CO)CCC3)c21]

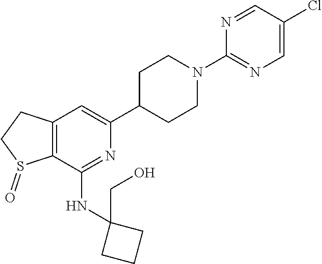 [SMILES: O=S1CCc2cc(C3CCN(c4ncc(Cl)cn4)CC3)nc(NC3(CO)CCC3)c21]

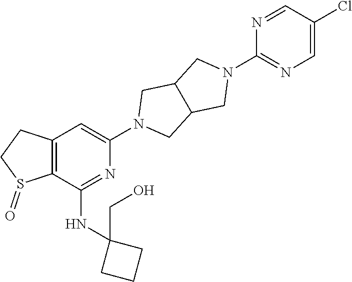 [SMILES: O=S1CCc2cc(N3CC4CN(c5ncc(Cl)cn5)CC4C3)nc(NC3(CO)CCC3)c21]

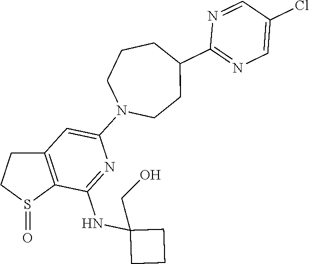 [SMILES: O=S1CCc2cc(N3CCCC(c4ncc(Cl)cn4)CC3)nc(NC3(CO)CCC3)c21]

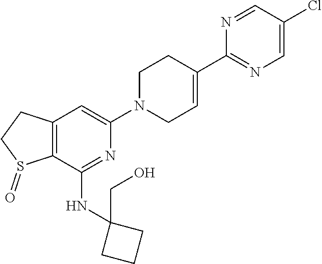 [SMILES: O=S1CCc2cc(N3CC=C(c4ncc(Cl)cn4)CC3)nc(NC3(CO)CCC3)c21]

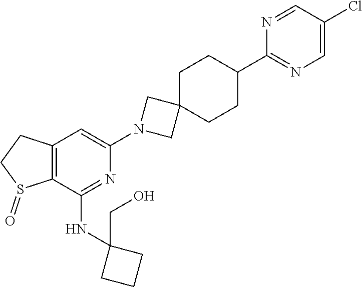 [SMILES: O=S1CCc2cc(N3CC4(CCC(c5ncc(Cl)cn5)CC4)C3)nc(NC3(CO)CCC3)c21]

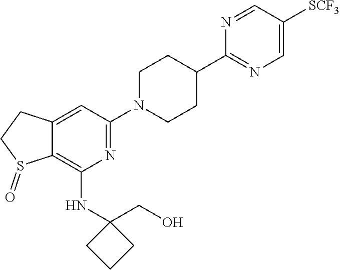

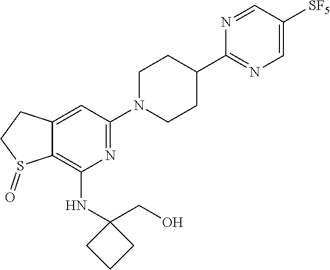

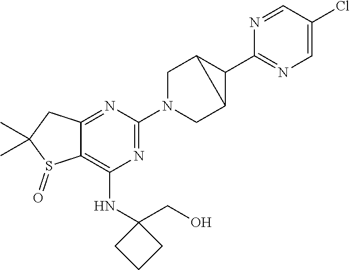 [SMILES: CC1(C)Cc2nc(N3CC4C(C3)C4c3ncc(Cl)cn3)nc(NC3(CO)CCC3)c2S1=O]

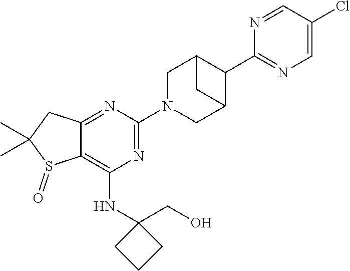 [SMILES: CC1(C)Cc2nc(N3CC4CC(C3)C4c3ncc(Cl)cn3)nc(NC3(CO)CCC3)c2S1=O]

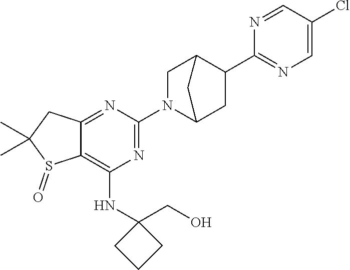 [SMILES: CC1(C)Cc2nc(N3CC4CC3CC4c3ncc(Cl)cn3)nc(NC3(CO)CCC3)c2S1=O]

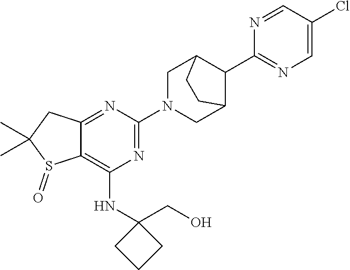 [SMILES: CC1(C)Cc2nc(N3CC4CCC(C3)C4c3ncc(Cl)cn3)nc(NC3(CO)CCC3)c2S1=O]

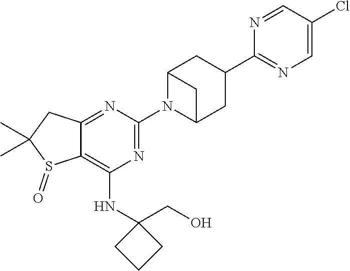 [SMILES: CC1(C)Cc2nc(N3C4CC(c5ncc(Cl)cn5)CC3C4)nc(NC3(CO)CCC3)c2S1=O]

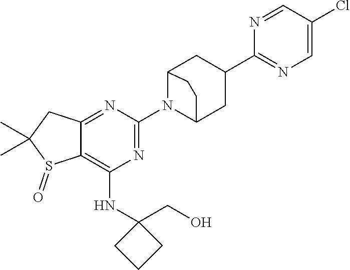 [SMILES: CC1(C)Cc2nc(N3C4CCC3CC(c3ncc(Cl)cn3)C4)nc(NC3(CO)CCC3)c2S1=O]

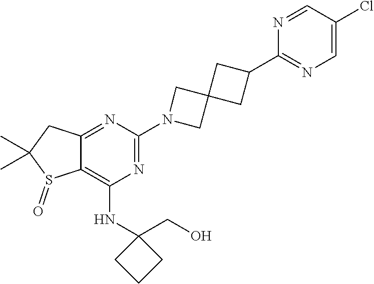 [SMILES: CC1(C)Cc2nc(N3CC4(CC(c5ncc(Cl)cn5)C4)C3)nc(NC3(CO)CCC3)c2S1=O]

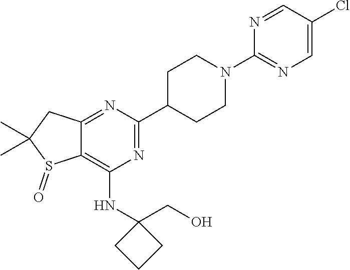 [SMILES: CC1(C)Cc2nc(C3CCN(c4ncc(Cl)cn4)CC3)nc(NC3(CO)CCC3)c2S1=O]

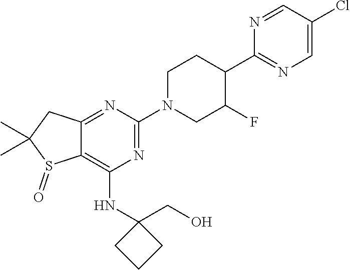 [SMILES: CC1(C)Cc2nc(N3CCC(c4ncc(Cl)cn4)C(F)C3)nc(NC3(CO)CCC3)c2S1=O]

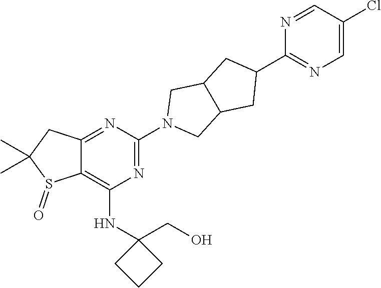 [SMILES: CC1(C)Cc2nc(N3CC4CC(c5ncc(Cl)cn5)CC4C3)nc(NC3(CO)CCC3)c2S1=O]

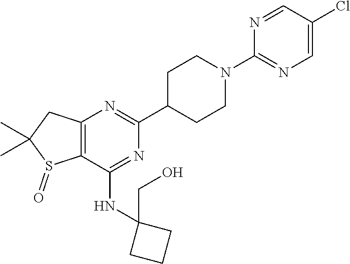 [SMILES: CC1(C)Cc2nc(C3CCN(c4ncc(Cl)cn4)CC3)nc(NC3(CO)CCC3)c2S1=O]

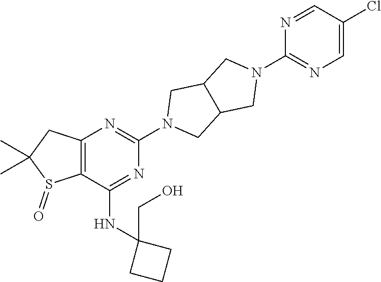 [SMILES: CC1(C)Cc2nc(N3CC4CN(c5ncc(Cl)cn5)CC4C3)nc(NC3(CO)CCC3)c2S1=O]

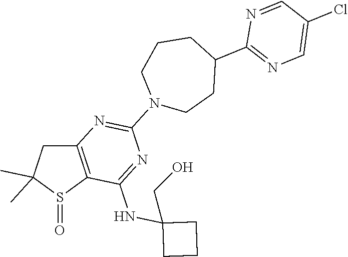 [SMILES: CC1(C)Cc2nc(N3CCCC(c4ncc(Cl)cn4)CC3)nc(NC3(CO)CCC3)c2S1=O]

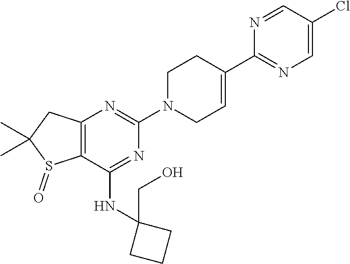 [SMILES: CC1(C)Cc2nc(N3CC=C(c4ncc(Cl)cn4)CC3)nc(NC3(CO)CCC3)c2S1=O]

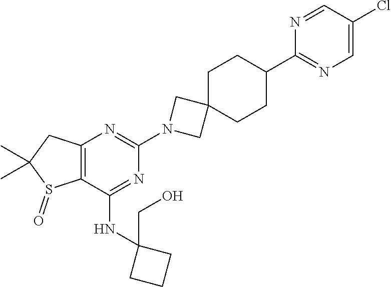 [SMILES: CC1(C)Cc2nc(N3CC4(CCC(c5ncc(Cl)cn5)CC4)C3)nc(NC3(CO)CCC3)c2S1=O]

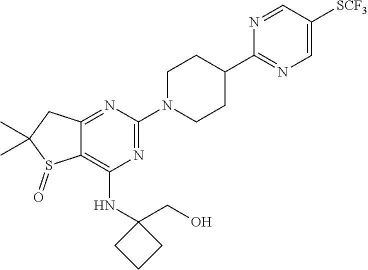

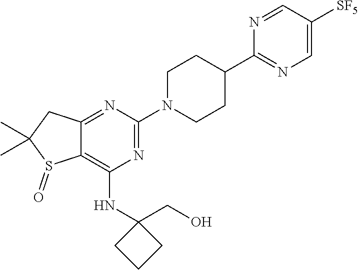

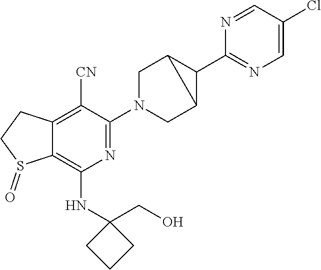 [SMILES: N#Cc1c(N2CC3C(C2)C3c2ncc(Cl)cn2)nc(NC2(CO)CCC2)c2c1CCS2=O]

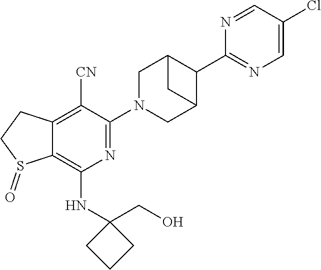 [SMILES: N#Cc1c(N2CC3CC(C2)C3c2ncc(Cl)cn2)nc(NC2(CO)CCC2)c2c1CCS2=O]

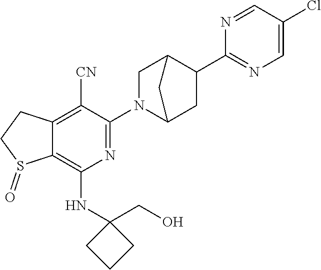 [SMILES: N#Cc1c(N2CC3CC2CC3c2ncc(Cl)cn2)nc(NC2(CO)CCC2)c2c1CCS2=O]

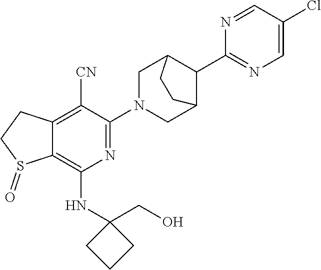 [SMILES: N#Cc1c(N2CC3CCC(C2)C3c2ncc(Cl)cn2)nc(NC2(CO)CCC2)c2c1CCS2=O]

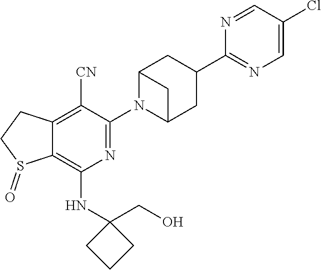 [SMILES: N#Cc1c(N2C3CC(c4ncc(Cl)cn4)CC2C3)nc(NC2(CO)CCC2)c2c1CCS2=O]

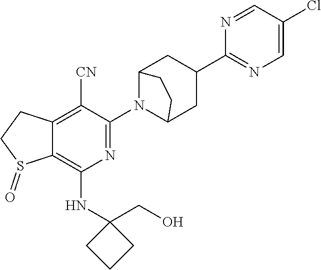 [SMILES: N#Cc1c(N2C3CCC2CC(c2ncc(Cl)cn2)C3)nc(NC2(CO)CCC2)c2c1CCS2=O]

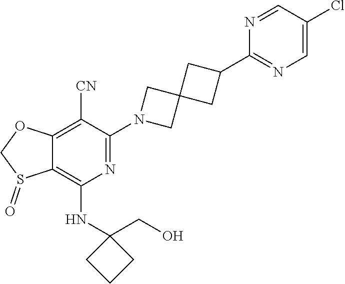 [SMILES: N#Cc1c(N2CC3(CC(c4ncc(Cl)cn4)C3)C2)nc(NC2(CO)CCC2)c2c1OCS2=O]

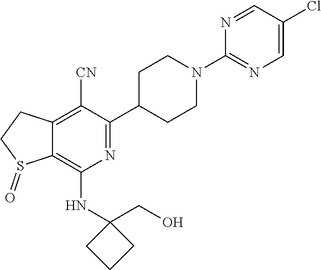 [SMILES: N#Cc1c(C2CCN(c3ncc(Cl)cn3)CC2)nc(NC2(CO)CCC2)c2c1CCS2=O]

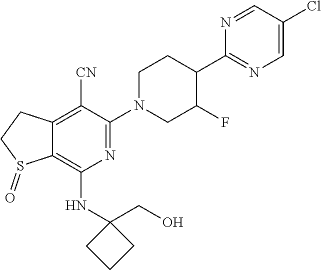 [SMILES: N#Cc1c(N2CCC(c3ncc(Cl)cn3)C(F)C2)nc(NC2(CO)CCC2)c2c1CCS2=O]

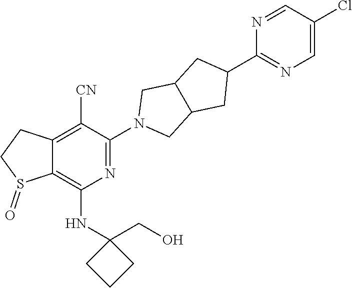 [SMILES: N#Cc1c(N2CC3CC(c4ncc(Cl)cn4)CC3C2)nc(NC2(CO)CCC2)c2c1CCS2=O]

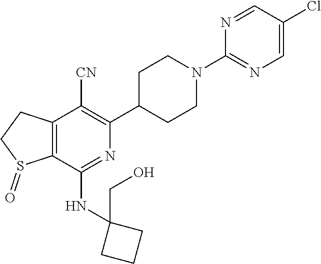 [SMILES: N#Cc1c(C2CCN(c3ncc(Cl)cn3)CC2)nc(NC2(CO)CCC2)c2c1CCS2=O]

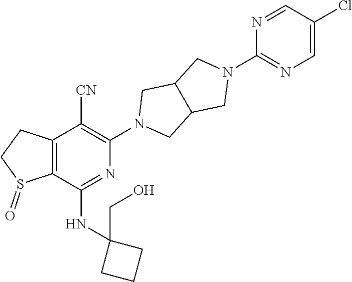 [SMILES: N#Cc1c(N2CC3CN(c4ncc(Cl)cn4)CC3C2)nc(NC2(CO)CCC2)c2c1CCS2=O]

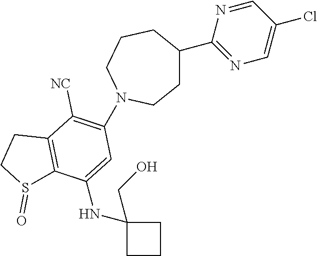 [SMILES: N#Cc1c(N2CCCC(c3ncc(Cl)cn3)CC2)cc(NC2(CO)CCC2)c2c1CCS2=O]

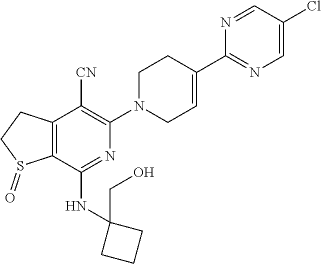 [SMILES: N#Cc1c(N2CC=C(c3ncc(Cl)cn3)CC2)nc(NC2(CO)CCC2)c2c1CCS2=O]

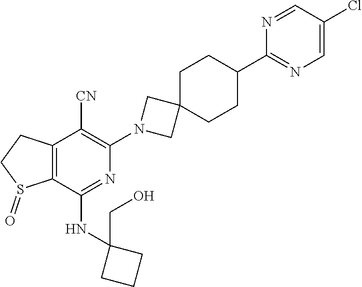 [SMILES: N#Cc1c(N2CC3(CCC(c4ncc(Cl)cn4)CC3)C2)nc(NC2(CO)CCC2)c2c1CCS2=O]

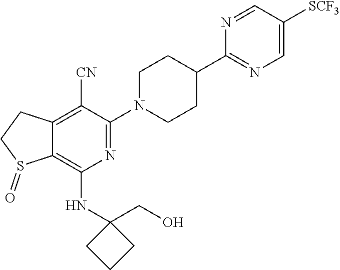

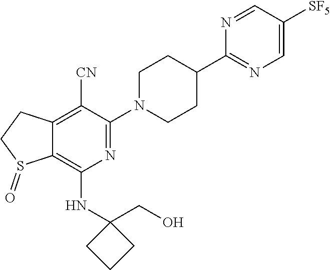

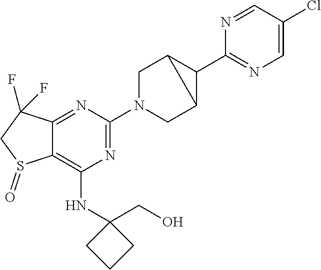 [SMILES: O=S1CC(F)(F)c2nc(N3CC4C(C3)C4c3ncc(Cl)cn3)nc(NC3(CO)CCC3)c21]

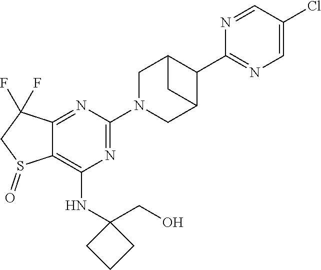 [SMILES: O=S1CC(F)(F)c2nc(N3CC4CC(C3)C4c3ncc(Cl)cn3)nc(NC3(CO)CCC3)c21]

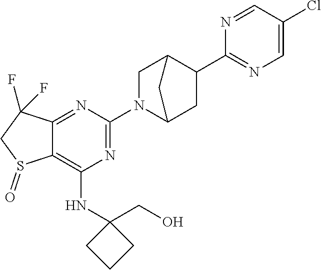 [SMILES: O=S1CC(F)(F)c2nc(N3CC4CC3CC4c3ncc(Cl)cn3)nc(NC3(CO)CCC3)c21]

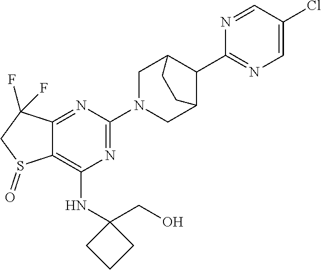 [SMILES: O=S1CC(F)(F)c2nc(N3CC4CCC(C3)C4c3ncc(Cl)cn3)nc(NC3(CO)CCC3)c21]

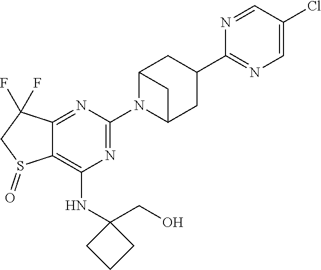 [SMILES: O=S1CC(F)(F)c2nc(N3C4CC(c5ncc(Cl)cn5)CC3C4)nc(NC3(CO)CCC3)c21]

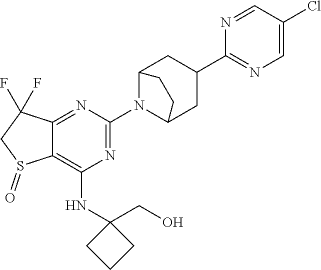 [SMILES: O=S1CC(F)(F)c2nc(N3C4CCC3CC(c3ncc(Cl)cn3)C4)nc(NC3(CO)CCC3)c21]

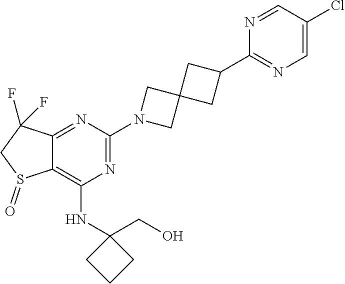 [SMILES: O=S1CC(F)(F)c2nc(N3CC4(CC(c5ncc(Cl)cn5)C4)C3)nc(NC3(CO)CCC3)c21]

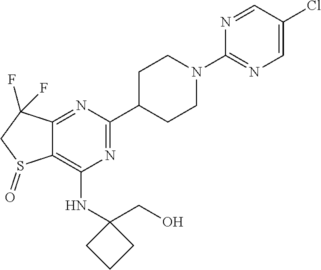 [SMILES: O=S1CC(F)(F)c2nc(C3CCN(c4ncc(Cl)cn4)CC3)nc(NC3(CO)CCC3)c21]

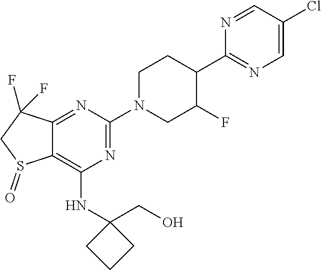 [SMILES: O=S1CC(F)(F)c2nc(N3CCC(c4ncc(Cl)cn4)C(F)C3)nc(NC3(CO)CCC3)c21]

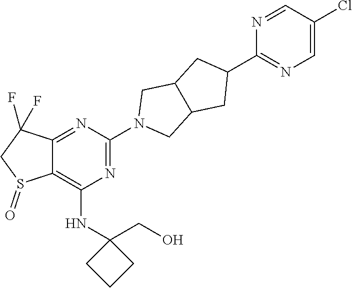 [SMILES: O=S1CC(F)(F)c2nc(N3CC4CC(c5ncc(Cl)cn5)CC4C3)nc(NC3(CO)CCC3)c21]

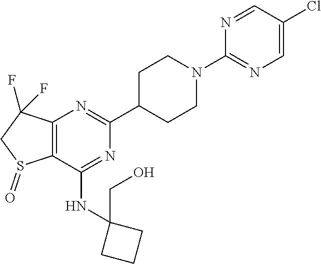 [SMILES: O=S1CC(F)(F)c2nc(C3CCN(c4ncc(Cl)cn4)CC3)nc(NC3(CO)CCC3)c21]

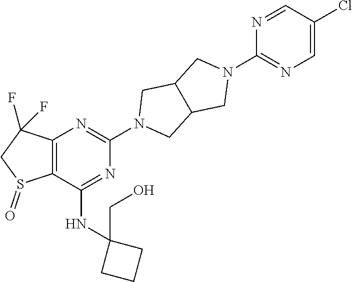 [SMILES: O=S1CC(F)(F)c2nc(N3CC4CN(c5ncc(Cl)cn5)CC4C3)nc(NC3(CO)CCC3)c21]

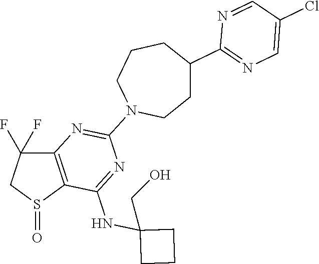 [SMILES: O=S1CC(F)(F)c2nc(N3CCCC(c4ncc(Cl)cn4)CC3)nc(NC3(CO)CCC3)c21]

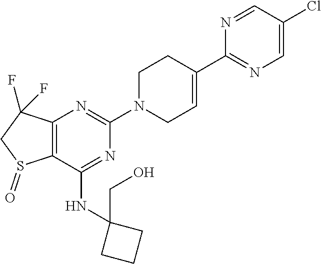 [SMILES: O=S1CC(F)(F)c2nc(N3CC=C(c4ncc(Cl)cn4)CC3)nc(NC3(CO)CCC3)c21]

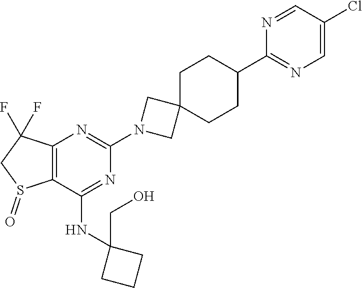 [SMILES: O=S1CC(F)(F)c2nc(N3CC4(CCC(c5ncc(Cl)cn5)CC4)C3)nc(NC3(CO)CCC3)c21]

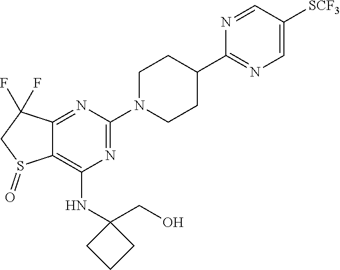

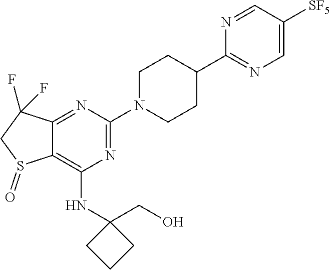

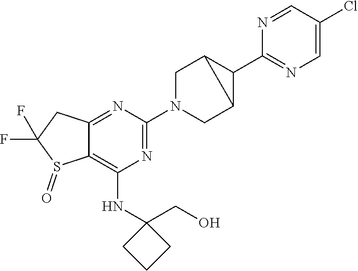 [SMILES: O=S1c2c(nc(N3CC4C(C3)C4c3ncc(Cl)cn3)nc2NC2(CO)CCC2)CC1(F)F]

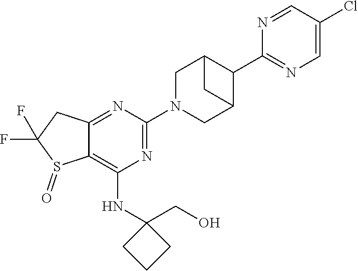 [SMILES: O=S1c2c(nc(N3CC4CC(C3)C4c3ncc(Cl)cn3)nc2NC2(CO)CCC2)CC1(F)F]

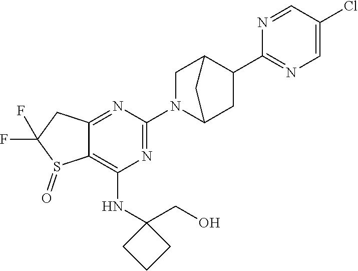 [SMILES: O=S1c2c(nc(N3CC4CC3CC4c3ncc(Cl)cn3)nc2NC2(CO)CCC2)CC1(F)F]

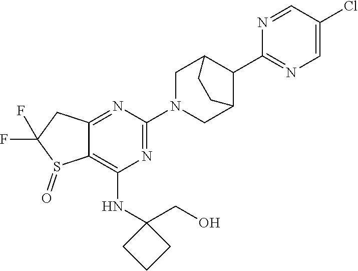 [SMILES: O=S1c2c(nc(N3CC4CCC(C3)C4c3ncc(Cl)cn3)nc2NC2(CO)CCC2)CC1(F)F]

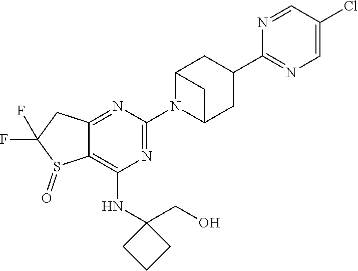 [SMILES: O=S1c2c(nc(N3C4CC(c5ncc(Cl)cn5)CC3C4)nc2NC2(CO)CCC2)CC1(F)F]

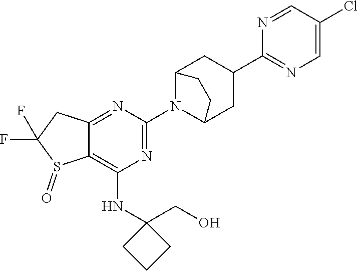 [SMILES: O=S1c2c(nc(N3C4CCC3CC(c3ncc(Cl)cn3)C4)nc2NC2(CO)CCC2)CC1(F)F]

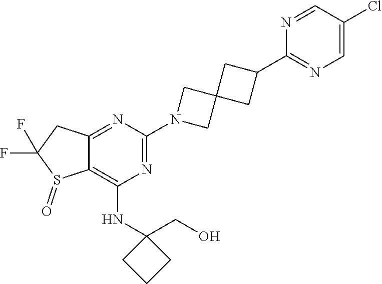 [SMILES: O=S1c2c(nc(N3CC4(CC(c5ncc(Cl)cn5)C4)C3)nc2NC2(CO)CCC2)CC1(F)F]

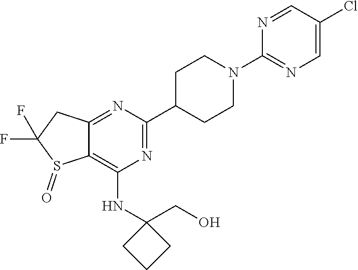 [SMILES: O=S1c2c(nc(C3CCN(c4ncc(Cl)cn4)CC3)nc2NC2(CO)CCC2)CC1(F)F]

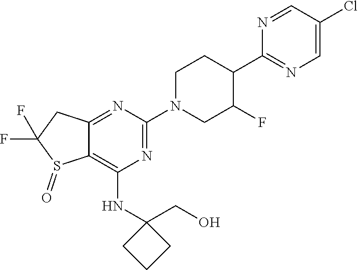 [SMILES: O=S1c2c(nc(N3CCC(c4ncc(Cl)cn4)C(F)C3)nc2NC2(CO)CCC2)CC1(F)F]

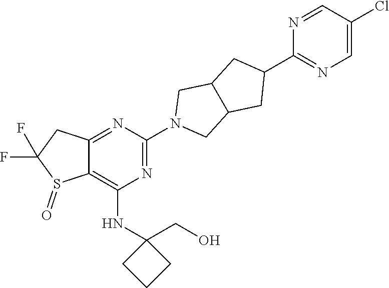 [SMILES: O=S1c2c(nc(N3CC4CC(c5ncc(Cl)cn5)CC4C3)nc2NC2(CO)CCC2)CC1(F)F]

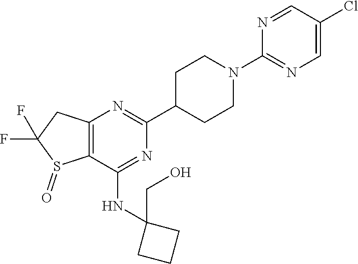 [SMILES: O=S1c2c(nc(C3CCN(c4ncc(Cl)cn4)CC3)nc2NC2(CO)CCC2)CC1(F)F]

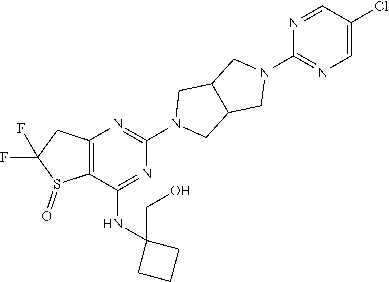 [SMILES: O=S1c2c(nc(N3CC4CN(c5ncc(Cl)cn5)CC4C3)nc2NC2(CO)CCC2)CC1(F)F]

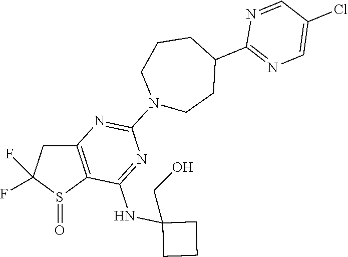 [SMILES: O=S1c2c(nc(N3CCCC(c4ncc(Cl)cn4)CC3)nc2NC2(CO)CCC2)CC1(F)F]

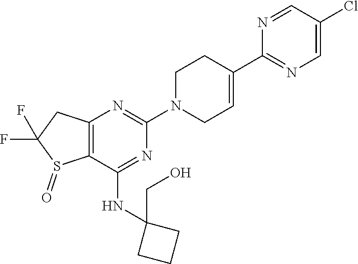 [SMILES: O=S1c2c(nc(N3CC=C(c4ncc(Cl)cn4)CC3)nc2NC2(CO)CCC2)CC1(F)F]

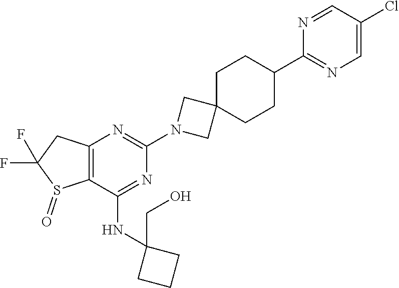 [SMILES: O=S1c2c(nc(N3CC4(CCC(c5ncc(Cl)cn5)CC4)C3)nc2NC2(CO)CCC2)CC1(F)F]

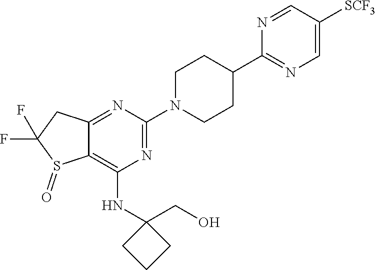

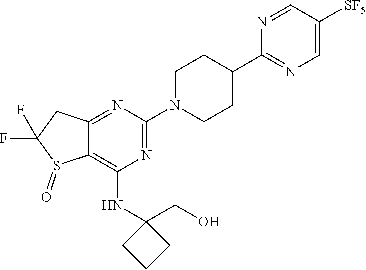

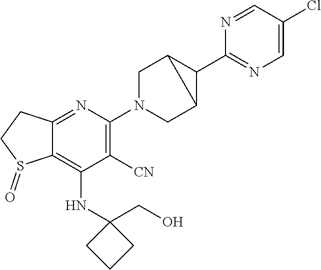 [SMILES: N#Cc1c(N2CC3C(C2)C3c2ncc(Cl)cn2)nc2c(c1NC1(CO)CCC1)S(=O)CC2]

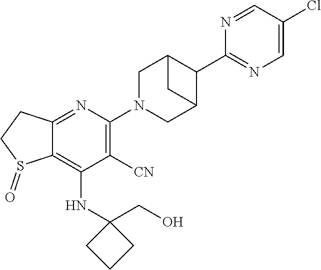 [SMILES: N#Cc1c(N2CC3CC(C2)C3c2ncc(Cl)cn2)nc2c(c1NC1(CO)CCC1)S(=O)CC2]

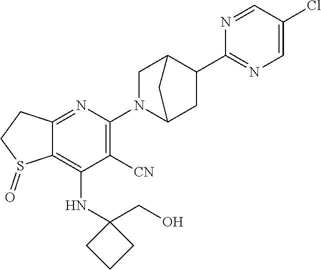 [SMILES: N#Cc1c(N2CC3CC2CC3c2ncc(Cl)cn2)nc2c(c1NC1(CO)CCC1)S(=O)CC2]

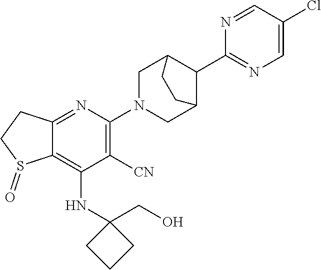 [SMILES: N#Cc1c(N2CC3CCC(C2)C3c2ncc(Cl)cn2)nc2c(c1NC1(CO)CCC1)S(=O)CC2]

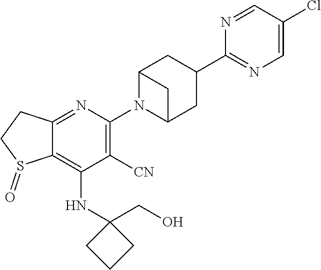 [SMILES: N#Cc1c(N2C3CC(c4ncc(Cl)cn4)CC2C3)nc2c(c1NC1(CO)CCC1)S(=O)CC2]

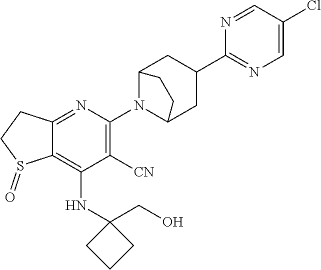 [SMILES: N#Cc1c(N2C3CCC2CC(c2ncc(Cl)cn2)C3)nc2c(c1NC1(CO)CCC1)S(=O)CC2]

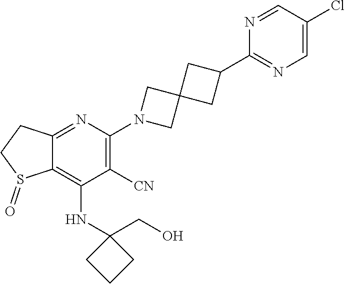 [SMILES: N#Cc1c(N2CC3(CC(c4ncc(Cl)cn4)C3)C2)nc2c(c1NC1(CO)CCC1)S(=O)CC2]

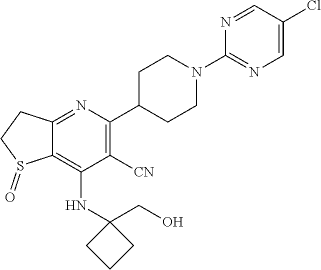 [SMILES: N#Cc1c(C2CCN(c3ncc(Cl)cn3)CC2)nc2c(c1NC1(CO)CCC1)S(=O)CC2]

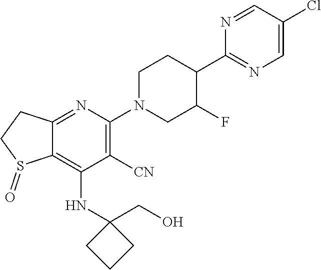 [SMILES: N#Cc1c(N2CCC(c3ncc(Cl)cn3)C(F)C2)nc2c(c1NC1(CO)CCC1)S(=O)CC2]

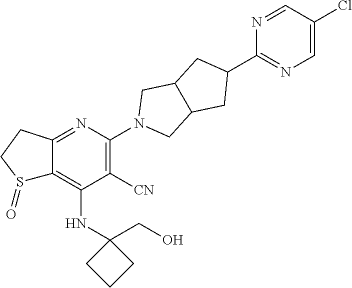 [SMILES: N#Cc1c(N2CC3CC(c4ncc(Cl)cn4)CC3C2)nc2c(c1NC1(CO)CCC1)S(=O)CC2]

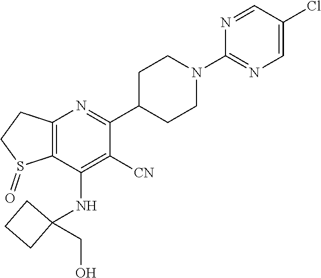 [SMILES: N#Cc1c(C2CCN(c3ncc(Cl)cn3)CC2)nc2c(c1NC1(CO)CCC1)S(=O)CC2]

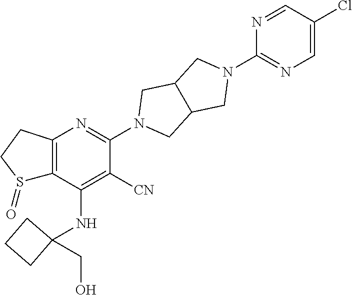 [SMILES: N#Cc1c(N2CC3CN(c4ncc(Cl)cn4)CC3C2)nc2c(c1NC1(CO)CCC1)S(=O)CC2]

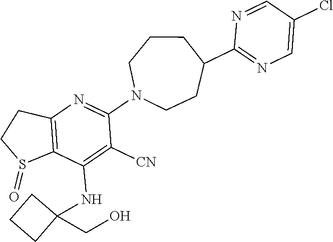 [SMILES: N#Cc1c(N2CCCC(c3ncc(Cl)cn3)CC2)nc2c(c1NC1(CO)CCC1)S(=O)CC2]

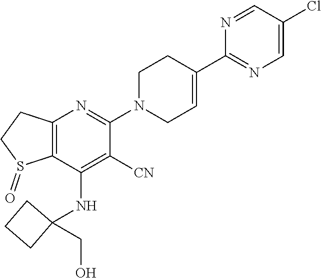 [SMILES: N#Cc1c(N2CC=C(c3ncc(Cl)cn3)CC2)nc2c(c1NC1(CO)CCC1)S(=O)CC2]

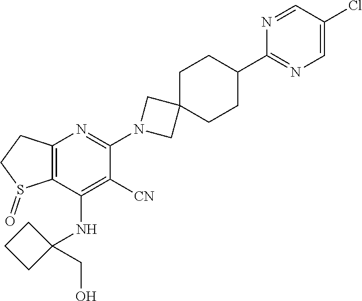 [SMILES: N#Cc1c(N2CC3(CCC(c4ncc(Cl)cn4)CC3)C2)nc2c(c1NC1(CO)CCC1)S(=O)CC2]

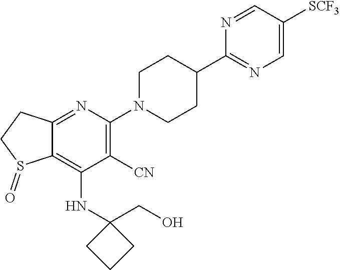

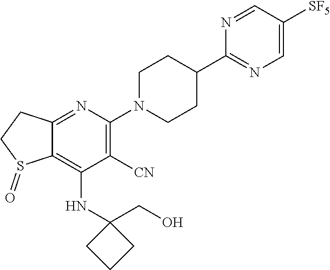

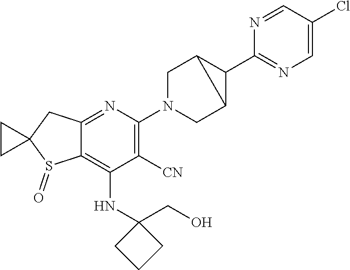 [SMILES: N#Cc1c(N2CC3C(C2)C3c2ncc(Cl)cn2)nc2c(c1NC1(CO)CCC1)S(=O)C1(CC1)C2]

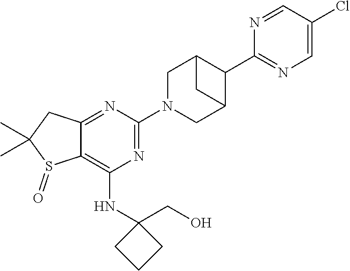 [SMILES: CC1(C)Cc2nc(N3CC4CC(C3)C4c3ncc(Cl)cn3)nc(NC3(CO)CCC3)c2S1=O]

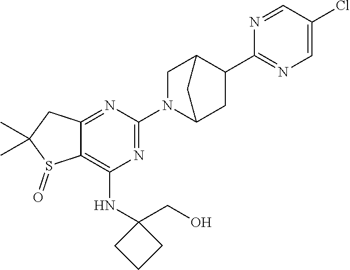 [SMILES: CC1(C)Cc2nc(N3CC4CC3CC4c3ncc(Cl)cn3)nc(NC3(CO)CCC3)c2S1=O]

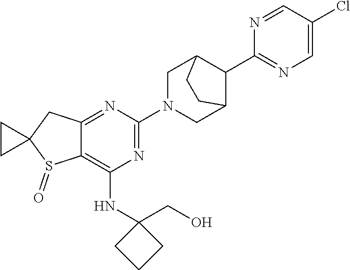 [SMILES: O=S1c2c(nc(N3CC4CCC(C3)C4c3ncc(Cl)cn3)nc2NC2(CO)CCC2)CC12CC2]

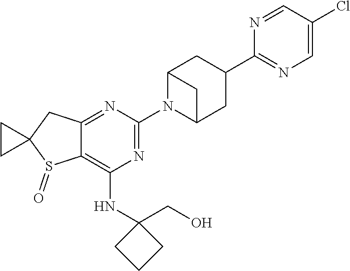 [SMILES: O=S1c2c(nc(N3C4CC(c5ncc(Cl)cn5)CC3C4)nc2NC2(CO)CCC2)CC12CC2]

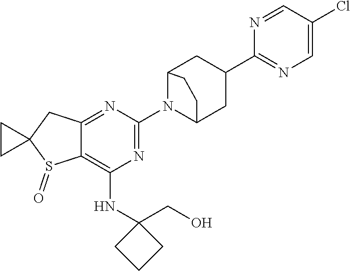 [SMILES: O=S1c2c(nc(N3C4CCC3CC(c3ncc(Cl)cn3)C4)nc2NC2(CO)CCC2)CC12CC2]

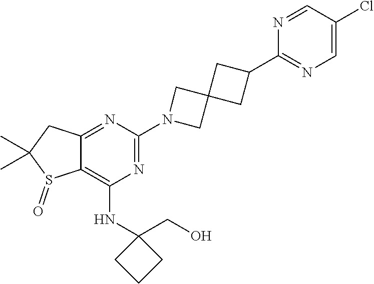 [SMILES: CC1(C)Cc2nc(N3CC4(CC(c5ncc(Cl)cn5)C4)C3)nc(NC3(CO)CCC3)c2S1=O]

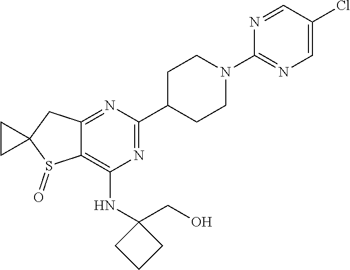 [SMILES: O=S1c2c(nc(C3CCN(c4ncc(Cl)cn4)CC3)nc2NC2(CO)CCC2)CC12CC2]

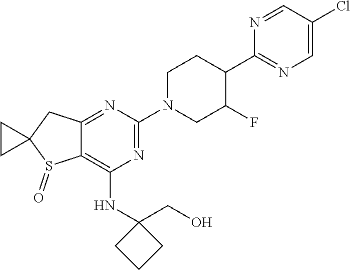 [SMILES: O=S1c2c(nc(N3CCC(c4ncc(Cl)cn4)C(F)C3)nc2NC2(CO)CCC2)CC12CC2]

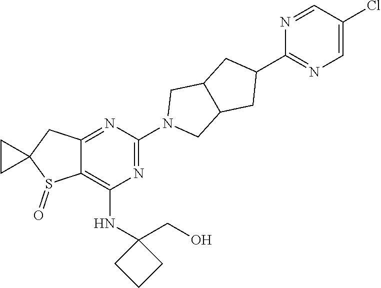 [SMILES: O=S1c2c(nc(N3CC4CC(c5ncc(Cl)cn5)CC4C3)nc2NC2(CO)CCC2)CC12CC2]

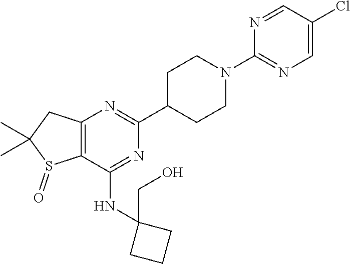 [SMILES: CC1(C)Cc2nc(C3CCN(c4ncc(Cl)cn4)CC3)nc(NC3(CO)CCC3)c2S1=O]

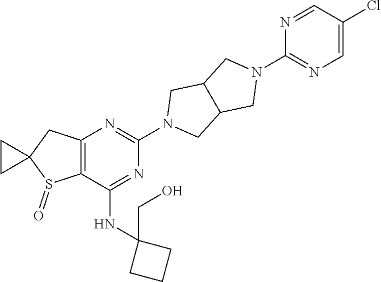 [SMILES: O=S1c2c(nc(N3CC4CN(c5ncc(Cl)cn5)CC4C3)nc2NC2(CO)CCC2)CC12CC2]

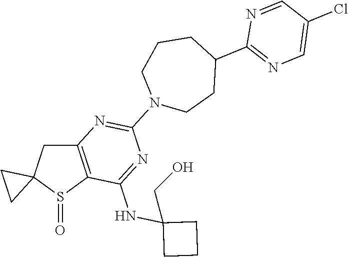 [SMILES: O=S1c2c(nc(N3CCCC(c4ncc(Cl)cn4)CC3)nc2NC2(CO)CCC2)CC12CC2]

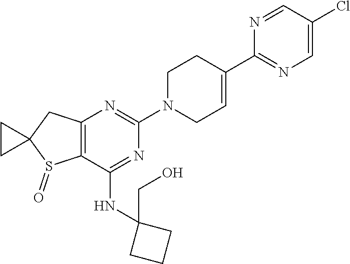 [SMILES: O=S1c2c(nc(N3CC=C(c4ncc(Cl)cn4)CC3)nc2NC2(CO)CCC2)CC12CC2]

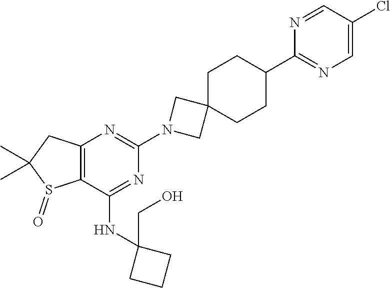 [SMILES: CC1(C)Cc2nc(N3CC4(CCC(c5ncc(Cl)cn5)CC4)C3)nc(NC3(CO)CCC3)c2S1=O]

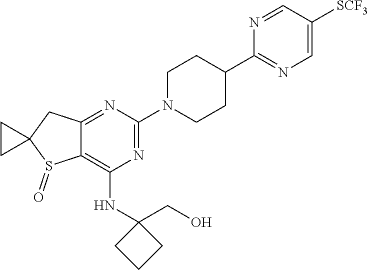

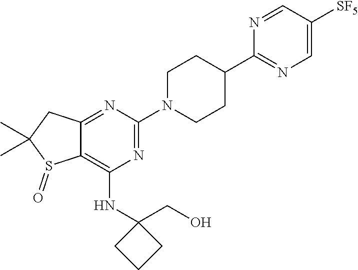

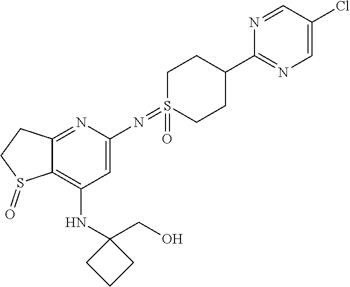 [SMILES: O=S1CCc2nc(N=S3(=O)CCC(c4ncc(Cl)cn4)CC3)cc(NC3(CO)CCC3)c21]

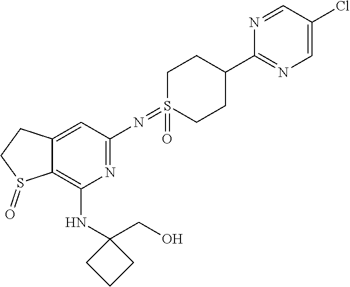 [SMILES: O=S1CCc2cc(N=S3(=O)CCC(c4ncc(Cl)cn4)CC3)nc(NC3(CO)CCC3)c21]

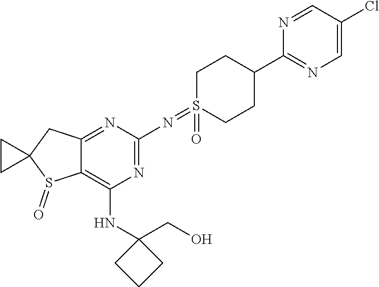 [SMILES: O=S1c2c(nc(N=S3(=O)CCC(c4ncc(Cl)cn4)CC3)nc2NC2(CO)CCC2)CC12CC2]

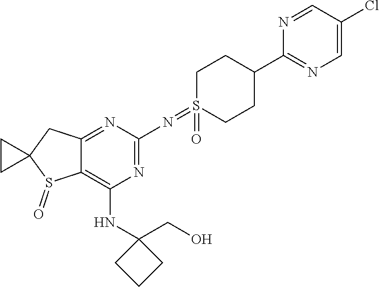 [SMILES: O=S1c2c(nc(N=S3(=O)CCC(c4ncc(Cl)cn4)CC3)nc2NC2(CO)CCC2)CC12CC2]

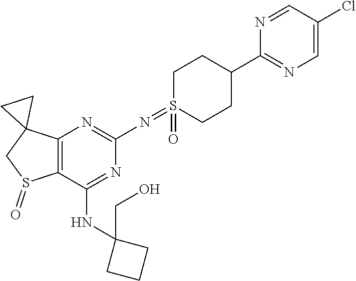 [SMILES: O=S1CC2(CC2)c2nc(N=S3(=O)CCC(c4ncc(Cl)cn4)CC3)nc(NC3(CO)CCC3)c21]

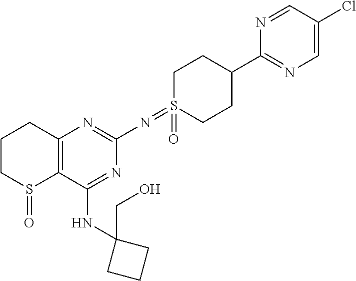 [SMILES: O=S1CCCc2nc(N=S3(=O)CCC(c4ncc(Cl)cn4)CC3)nc(NC3(CO)CCC3)c21]

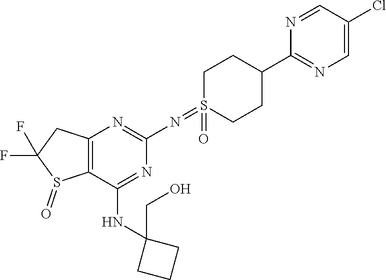 [SMILES: O=S1c2c(nc(N=S3(=O)CCC(c4ncc(Cl)cn4)CC3)nc2NC2(CO)CCC2)CC1(F)F]

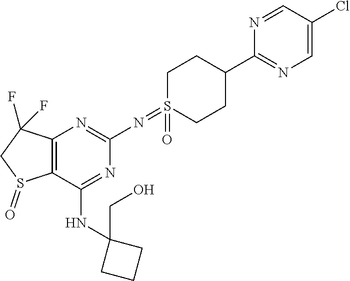 [SMILES: O=S1CC(F)(F)c2nc(N=S3(=O)CCC(c4ncc(Cl)cn4)CC3)nc(NC3(CO)CCC3)c21]

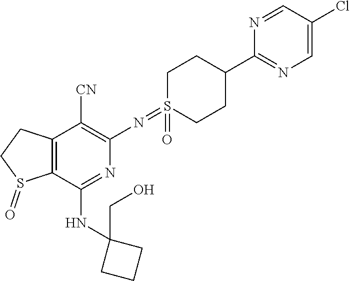 [SMILES: N#Cc1c(N=S2(=O)CCC(c3ncc(Cl)cn3)CC2)nc(NC2(CO)CCC2)c2c1CCS2=O]

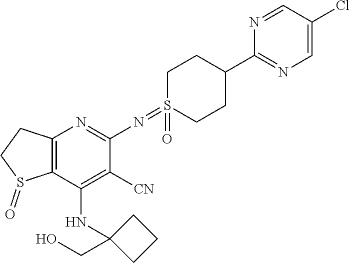 [SMILES: N#Cc1c(N=S2(=O)CCC(c3ncc(Cl)cn3)CC2)nc2c(c1NC1(CO)CCC1)S(=O)CC2]

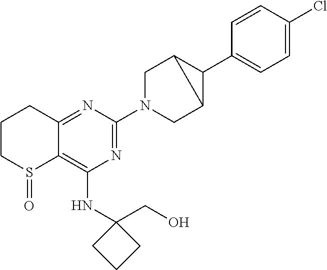 [SMILES: O=S1CCCc2nc(N3CC4C(C3)C4c3ccc(Cl)cc3)nc(NC3(CO)CCC3)c21]

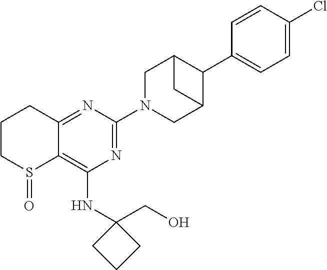 [SMILES: O=S1CCCc2nc(N3CC4CC(C3)C4c3ccc(Cl)cc3)nc(NC3(CO)CCC3)c21]

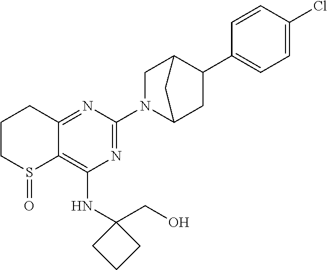 [SMILES: O=S1CCCc2nc(N3CC4CC3CC4c3ccc(Cl)cc3)nc(NC3(CO)CCC3)c21]

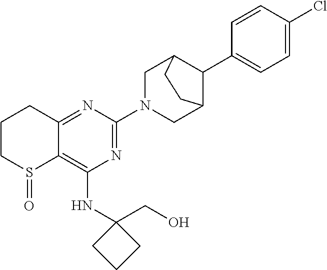 [SMILES: O=S1CCCc2nc(N3CC4CCC(C3)C4c3ccc(Cl)cc3)nc(NC3(CO)CCC3)c21]

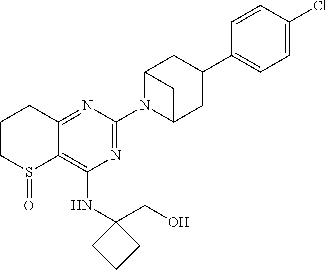 [SMILES: O=S1CCCc2nc(N3C4CC(c5ccc(Cl)cc5)CC3C4)nc(NC3(CO)CCC3)c21]

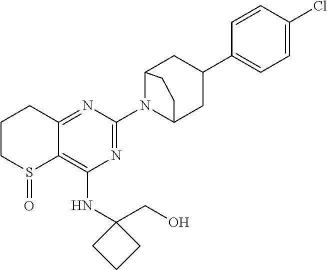 [SMILES: O=S1CCCc2nc(N3C4CCC3CC(c3ccc(Cl)cc3)C4)nc(NC3(CO)CCC3)c21]

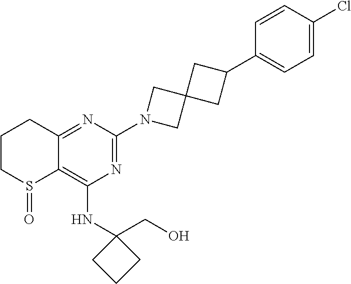 [SMILES: O=S1CCCc2nc(N3CC4(CC(c5ccc(Cl)cc5)C4)C3)nc(NC3(CO)CCC3)c21]

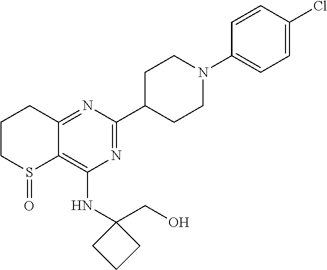 [SMILES: O=S1CCCc2nc(C3CCN(c4ccc(Cl)cc4)CC3)nc(NC3(CO)CCC3)c21]

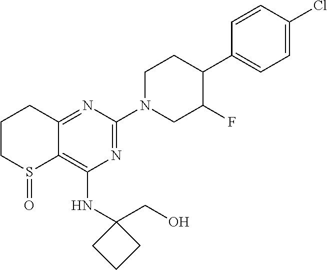 [SMILES: O=S1CCCc2nc(N3CCC(c4ccc(Cl)cc4)C(F)C3)nc(NC3(CO)CCC3)c21]

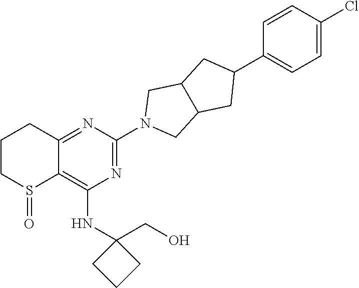 [SMILES: O=S1CCCc2nc(N3CC4CC(c5ccc(Cl)cc5)CC4C3)nc(NC3(CO)CCC3)c21]

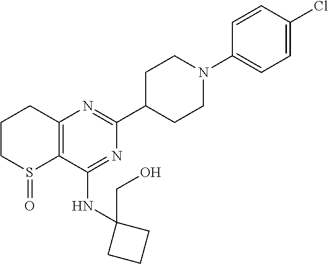 [SMILES: O=S1CCCc2nc(C3CCN(c4ccc(Cl)cc4)CC3)nc(NC3(CO)CCC3)c21]

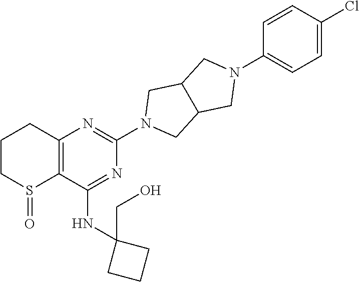 [SMILES: O=S1CCCc2nc(N3CC4CN(c5ccc(Cl)cc5)CC4C3)nc(NC3(CO)CCC3)c21]

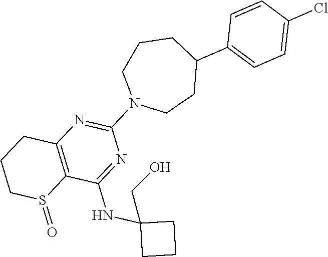 [SMILES: O=S1CCCc2nc(N3CCCC(c4ccc(Cl)cc4)CC3)nc(NC3(CO)CCC3)c21]

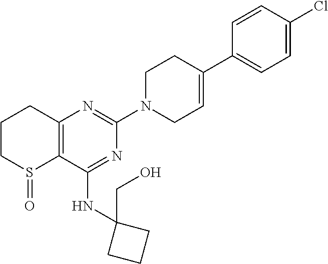 [SMILES: O=S1CCCc2nc(N3CC=C(c4ccc(Cl)cc4)CC3)nc(NC3(CO)CCC3)c21]

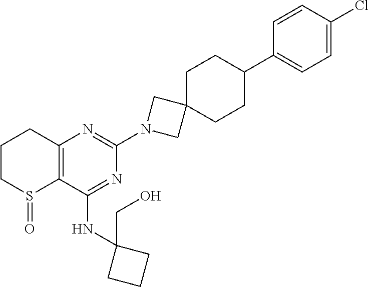 [SMILES: O=S1CCCc2nc(N3CC4(CCC(c5ccc(Cl)cc5)CC4)C3)nc(NC3(CO)CCC3)c21]

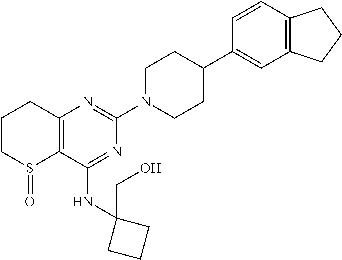 [SMILES: O=S1CCCc2nc(N3CCC(c4ccc5c(c4)CCC5)CC3)nc(NC3(CO)CCC3)c21]

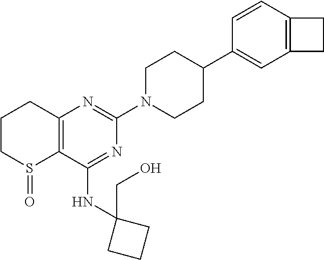 [SMILES: O=S1CCCc2nc(N3CCC(c4ccc5c(c4)CC5)CC3)nc(NC3(CO)CCC3)c21]

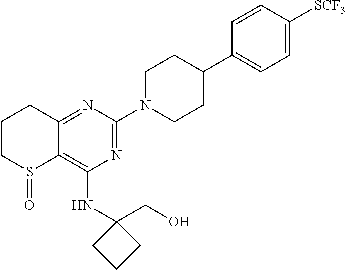

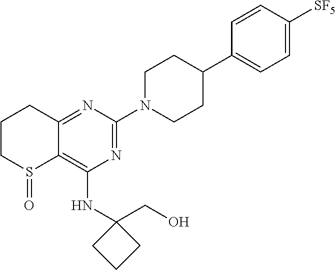

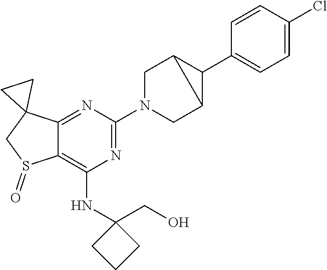 [SMILES: O=S1CC2(CC2)c2nc(N3CC4C(C3)C4c3ccc(Cl)cc3)nc(NC3(CO)CCC3)c21]

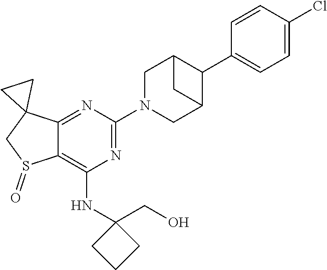 [SMILES: O=S1CC2(CC2)c2nc(N3CC4CC(C3)C4c3ccc(Cl)cc3)nc(NC3(CO)CCC3)c21]

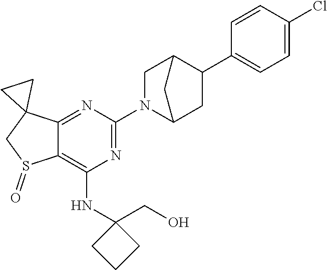 [SMILES: O=S1CC2(CC2)c2nc(N3CC4CC3CC4c3ccc(Cl)cc3)nc(NC3(CO)CCC3)c21]

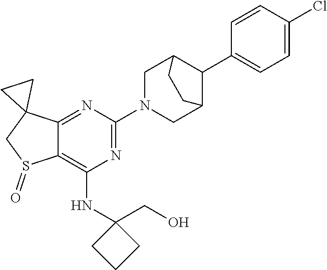 [SMILES: O=S1CC2(CC2)c2nc(N3CC4CCC(C3)C4c3ccc(Cl)cc3)nc(NC3(CO)CCC3)c21]

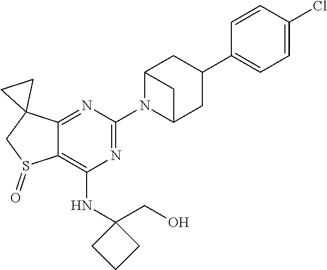 [SMILES: O=S1CC2(CC2)c2nc(N3C4CC(c5ccc(Cl)cc5)CC3C4)nc(NC3(CO)CCC3)c21]

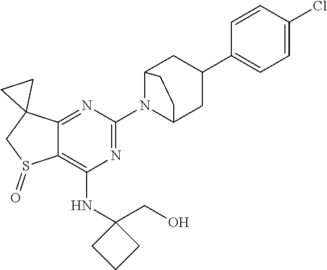 [SMILES: O=S1CC2(CC2)c2nc(N3C4CCC3CC(c3ccc(Cl)cc3)C4)nc(NC3(CO)CCC3)c21]

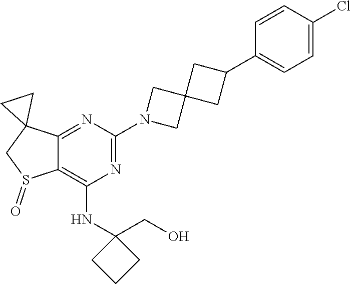 [SMILES: O=S1CC2(CC2)c2nc(N3CC4(CC(c5ccc(Cl)cc5)C4)C3)nc(NC3(CO)CCC3)c21]

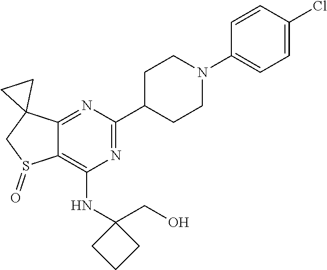 [SMILES: O=S1CC2(CC2)c2nc(C3CCN(c4ccc(Cl)cc4)CC3)nc(NC3(CO)CCC3)c21]

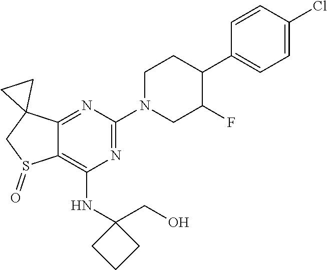 [SMILES: O=S1CC2(CC2)c2nc(N3CCC(c4ccc(Cl)cc4)C(F)C3)nc(NC3(CO)CCC3)c21]

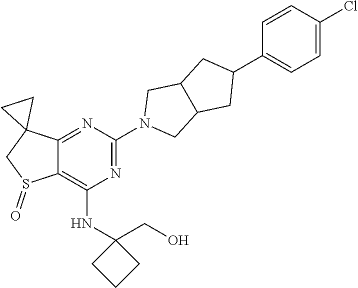 [SMILES: O=S1CC2(CC2)c2nc(N3CC4CC(c5ccc(Cl)cc5)CC4C3)nc(NC3(CO)CCC3)c21]

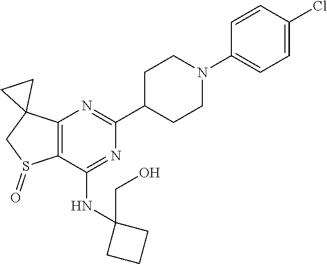 [SMILES: O=S1CC2(CC2)c2nc(C3CCN(c4ccc(Cl)cc4)CC3)nc(NC3(CO)CCC3)c21]

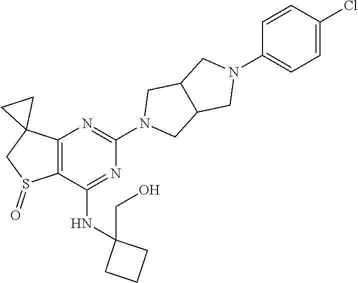 [SMILES: O=S1CC2(CC2)c2nc(N3CC4CN(c5ccc(Cl)cc5)CC4C3)nc(NC3(CO)CCC3)c21]

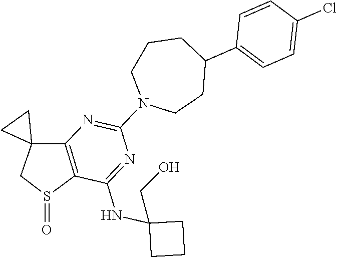 [SMILES: O=S1CC2(CC2)c2nc(N3CCCC(c4ccc(Cl)cc4)CC3)nc(NC3(CO)CCC3)c21]

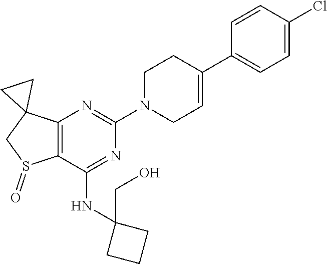 [SMILES: O=S1CC2(CC2)c2nc(N3CC=C(c4ccc(Cl)cc4)CC3)nc(NC3(CO)CCC3)c21]

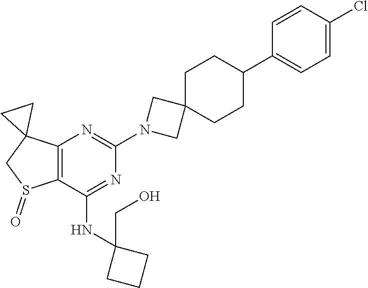 [SMILES: O=S1CC2(CC2)c2nc(N3CC4(CCC(c5ccc(Cl)cc5)CC4)C3)nc(NC3(CO)CCC3)c21]

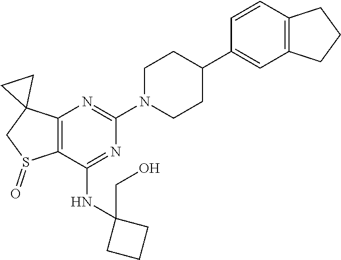 [SMILES: O=S1CC2(CC2)c2nc(N3CCC(c4ccc5c(c4)CCC5)CC3)nc(NC3(CO)CCC3)c21]

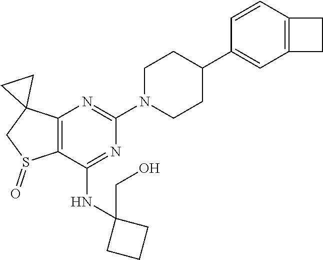 [SMILES: O=S1CC2(CC2)c2nc(N3CCC(c4ccc5c(c4)CC5)CC3)nc(NC3(CO)CCC3)c21]

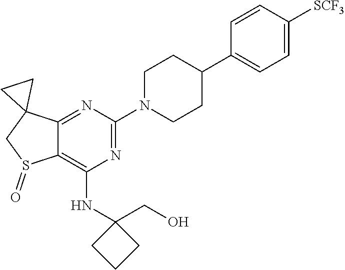

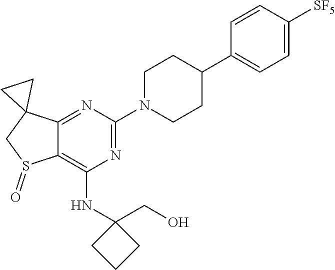

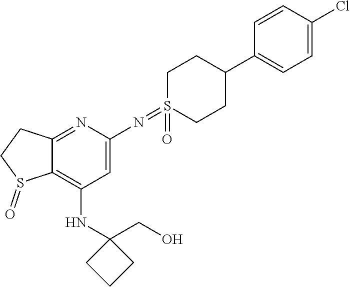 [SMILES: O=S1CCc2nc(N=S3(=O)CCC(c4ccc(Cl)cc4)CC3)cc(NC3(CO)CCC3)c21]

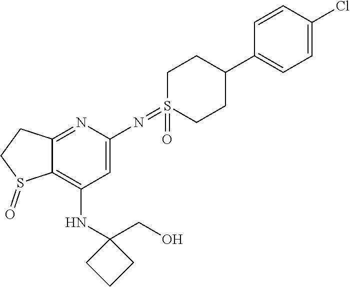 [SMILES: O=S1CCc2nc(N=S3(=O)CCC(c4ccc(Cl)cc4)CC3)cc(NC3(CO)CCC3)c21]

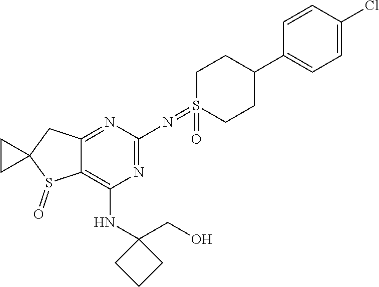 [SMILES: O=S1c2c(nc(N=S3(=O)CCC(c4ccc(Cl)cc4)CC3)nc2NC2(CO)CCC2)CC12CC2]

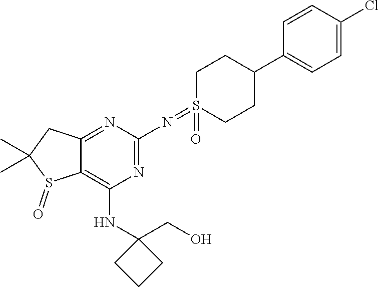 [SMILES: CC1(C)Cc2nc(N=S3(=O)CCC(c4ccc(Cl)cc4)CC3)nc(NC3(CO)CCC3)c2S1=O]

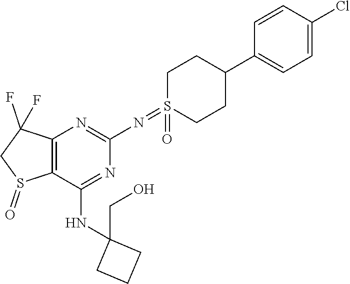 [SMILES: O=S1CC(F)(F)c2nc(N=S3(=O)CCC(c4ccc(Cl)cc4)CC3)nc(NC3(CO)CCC3)c21]

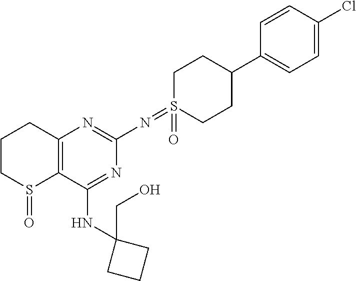 [SMILES: O=S1CCCc2nc(N=S3(=O)CCC(c4ccc(Cl)cc4)CC3)nc(NC3(CO)CCC3)c21]

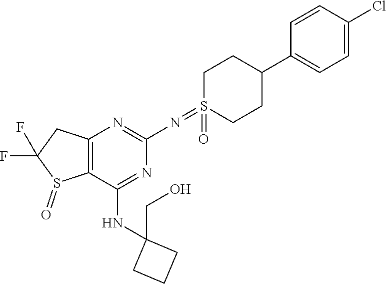 [SMILES: O=S1c2c(nc(N=S3(=O)CCC(c4ccc(Cl)cc4)CC3)nc2NC2(CO)CCC2)CC1(F)F]

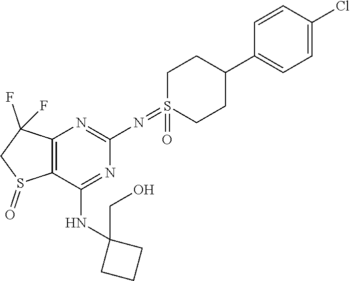 [SMILES: O=S1CC(F)(F)c2nc(N=S3(=O)CCC(c4ccc(Cl)cc4)CC3)nc(NC3(CO)CCC3)c21]

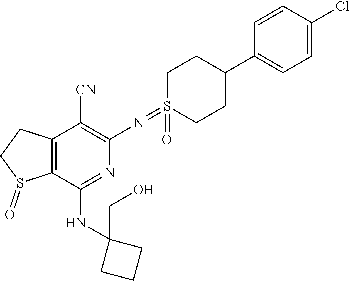 [SMILES: N#Cc1c(N=S2(=O)CCC(c3ccc(Cl)cc3)CC2)nc(NC2(CO)CCC2)c2c1CCS2=O]

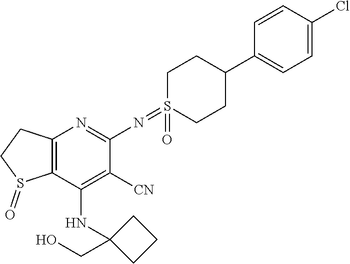 [SMILES: N#Cc1c(N=S2(=O)CCC(c3ccc(Cl)cc3)CC2)nc2c(c1NC1(CO)CCC1)S(=O)CC2]

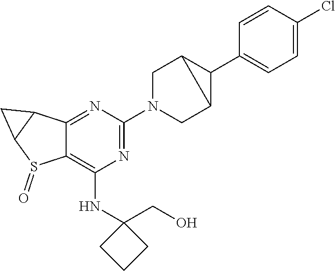 [SMILES: O=S1c2c(NC3(CO)CCC3)nc(N3CC4C(C3)C4c3ccc(Cl)cc3)nc2C2CC21]

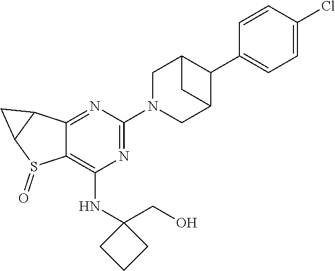 [SMILES: O=S1c2c(NC3(CO)CCC3)nc(N3CC4CC(C3)C4c3ccc(Cl)cc3)nc2C2CC21]

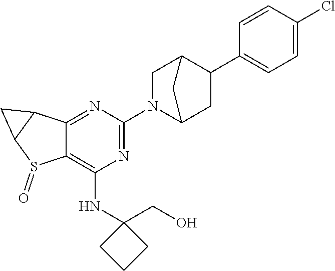 [SMILES: O=S1c2c(NC3(CO)CCC3)nc(N3CC4CC3CC4c3ccc(Cl)cc3)nc2C2CC21]

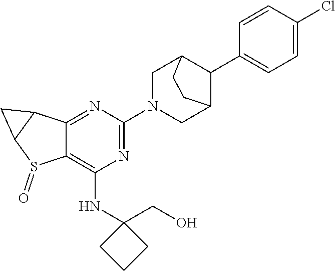 [SMILES: O=S1c2c(NC3(CO)CCC3)nc(N3CC4CCC(C3)C4c3ccc(Cl)cc3)nc2C2CC21]

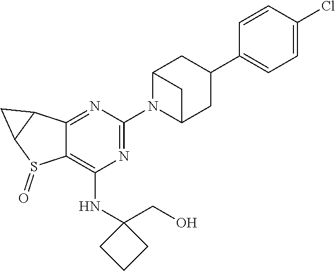 [SMILES: O=S1c2c(NC3(CO)CCC3)nc(N3C4CC(c5ccc(Cl)cc5)CC3C4)nc2C2CC21]

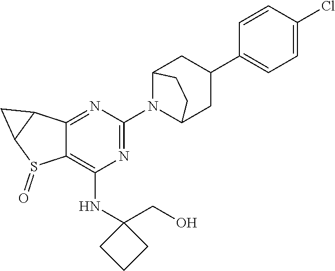 [SMILES: O=S1c2c(NC3(CO)CCC3)nc(N3C4CCC3CC(c3ccc(Cl)cc3)C4)nc2C2CC21]

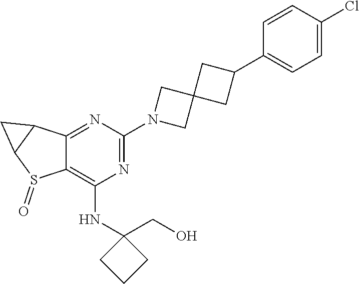 [SMILES: O=S1c2c(NC3(CO)CCC3)nc(N3CC4(CC(c5ccc(Cl)cc5)C4)C3)nc2C2CC21]

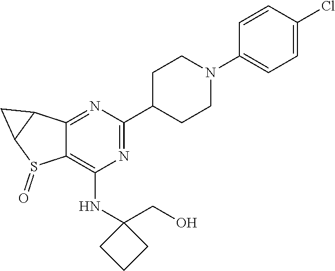 [SMILES: O=S1c2c(NC3(CO)CCC3)nc(C3CCN(c4ccc(Cl)cc4)CC3)nc2C2CC21]

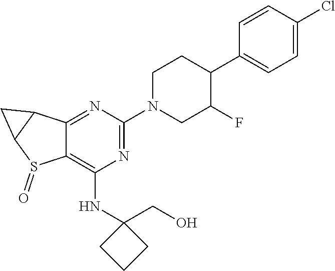 [SMILES: O=S1c2c(NC3(CO)CCC3)nc(N3CCC(c4ccc(Cl)cc4)C(F)C3)nc2C2CC21]

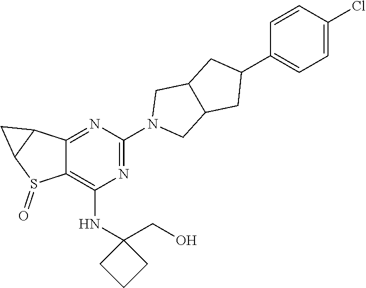 [SMILES: O=S1c2c(NC3(CO)CCC3)nc(N3CC4CC(c5ccc(Cl)cc5)CC4C3)nc2C2CC21]

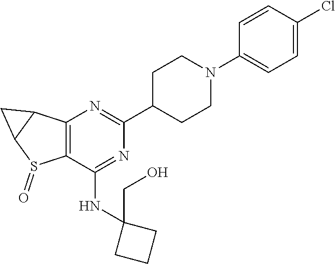 [SMILES: O=S1c2c(NC3(CO)CCC3)nc(C3CCN(c4ccc(Cl)cc4)CC3)nc2C2CC21]

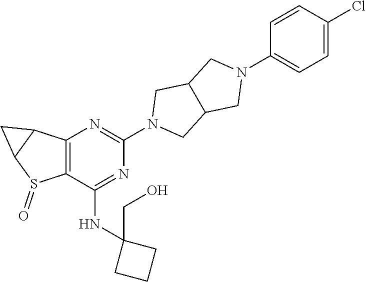 [SMILES: O=S1c2c(NC3(CO)CCC3)nc(N3CC4CN(c5ccc(Cl)cc5)CC4C3)nc2C2CC21]

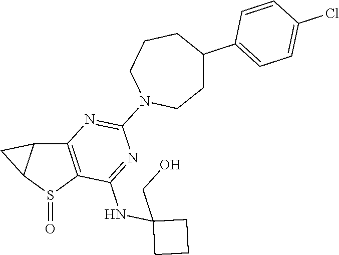 [SMILES: O=S1c2c(NC3(CO)CCC3)nc(N3CCCC(c4ccc(Cl)cc4)CC3)nc2C2CC21]

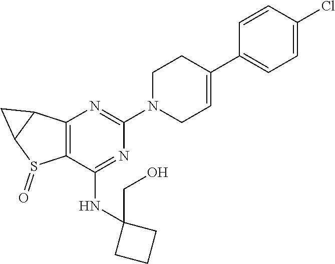 [SMILES: O=S1c2c(NC3(CO)CCC3)nc(N3CC=C(c4ccc(Cl)cc4)CC3)nc2C2CC21]

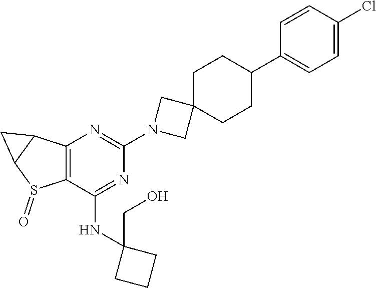 [SMILES: O=S1c2c(NC3(CO)CCC3)nc(N3CC4(CCC(c5ccc(Cl)cc5)CC4)C3)nc2C2CC21]

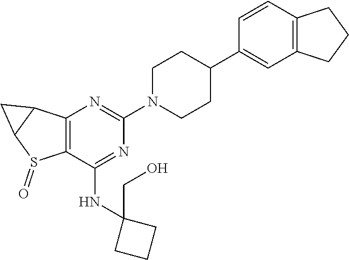 [SMILES: O=S1c2c(NC3(CO)CCC3)nc(N3CCC(c4ccc5c(c4)CCC5)CC3)nc2C2CC21]

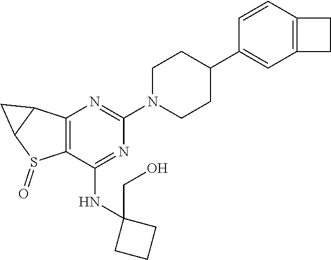 [SMILES: O=S1c2c(NC3(CO)CCC3)nc(N3CCC(c4ccc5c(c4)CC5)CC3)nc2C2CC21]

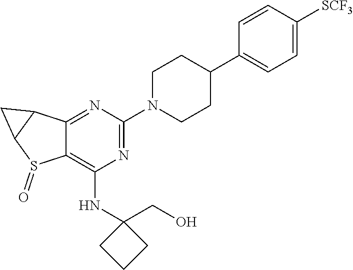

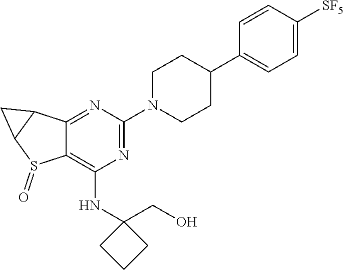

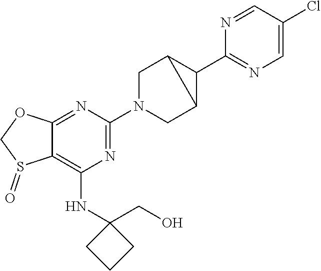 [SMILES: O=S1COc2nc(N3CC4C(C3)C4c3ncc(Cl)cn3)nc(NC3(CO)CCC3)c21]

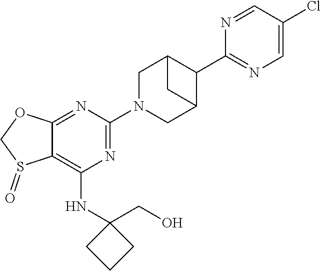 [SMILES: O=S1COc2nc(N3CC4CC(C3)C4c3ncc(Cl)cn3)nc(NC3(CO)CCC3)c21]

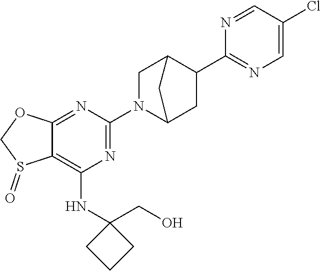 [SMILES: O=S1COc2nc(N3CC4CC3CC4c3ncc(Cl)cn3)nc(NC3(CO)CCC3)c21]

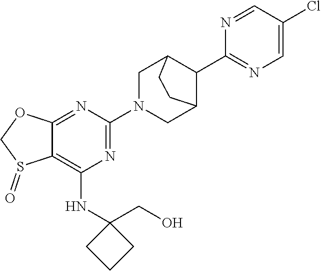 [SMILES: O=S1COc2nc(N3CC4CCC(C3)C4c3ncc(Cl)cn3)nc(NC3(CO)CCC3)c21]

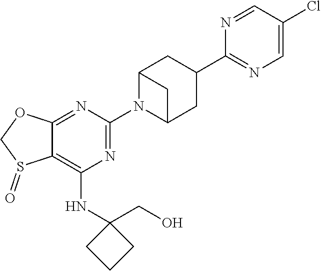 [SMILES: O=S1COc2nc(N3C4CC(c5ncc(Cl)cn5)CC3C4)nc(NC3(CO)CCC3)c21]

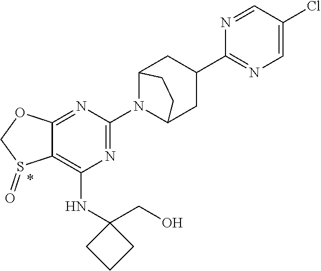 [SMILES: O=S1COc2nc(N3C4CCC3CC(c3ncc(Cl)cn3)C4)nc(NC3(CO)CCC3)c21]

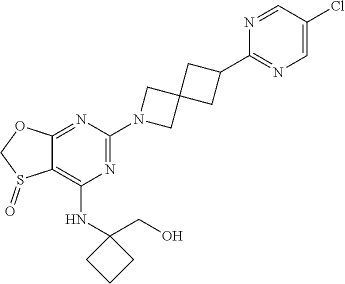 [SMILES: O=S1COc2nc(N3CC4(CC(c5ncc(Cl)cn5)C4)C3)nc(NC3(CO)CCC3)c21]

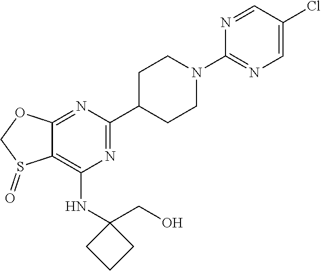 [SMILES: O=S1COc2nc(C3CCN(c4ncc(Cl)cn4)CC3)nc(NC3(CO)CCC3)c21]

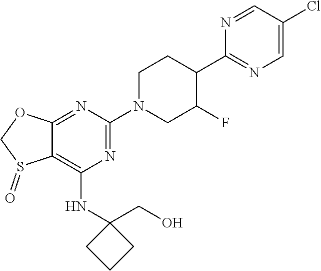 [SMILES: O=S1COc2nc(N3CCC(c4ncc(Cl)cn4)C(F)C3)nc(NC3(CO)CCC3)c21]

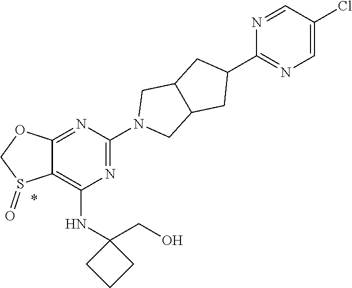 [SMILES: O=S1COc2nc(N3CC4CC(c5ncc(Cl)cn5)CC4C3)nc(NC3(CO)CCC3)c21]

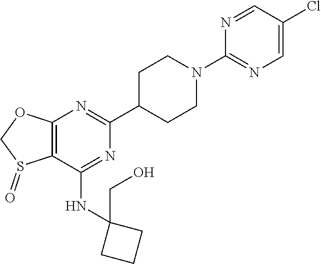 [SMILES: O=S1COc2nc(C3CCN(c4ncc(Cl)cn4)CC3)nc(NC3(CO)CCC3)c21]

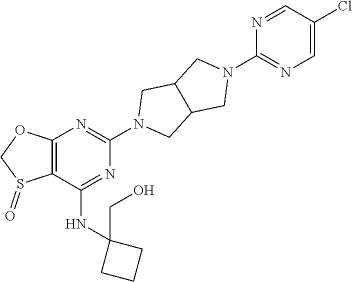 [SMILES: O=S1COc2nc(N3CC4CN(c5ncc(Cl)cn5)CC4C3)nc(NC3(CO)CCC3)c21]

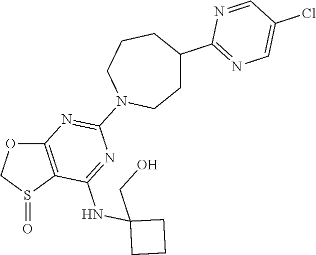 [SMILES: O=S1COc2nc(N3CCCC(c4ncc(Cl)cn4)CC3)nc(NC3(CO)CCC3)c21]

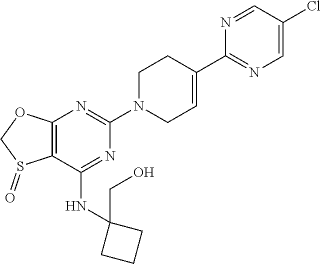 [SMILES: O=S1COc2nc(N3CC=C(c4ncc(Cl)cn4)CC3)nc(NC3(CO)CCC3)c21]

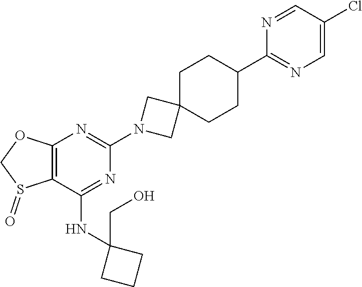 [SMILES: O=S1COc2nc(N3CC4(CCC(c5ncc(Cl)cn5)CC4)C3)nc(NC3(CO)CCC3)c21]

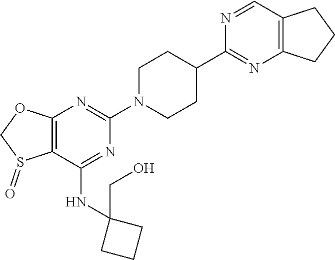 [SMILES: O=S1COc2nc(N3CCC(c4ncc5c(n4)CCC5)CC3)nc(NC3(CO)CCC3)c21]

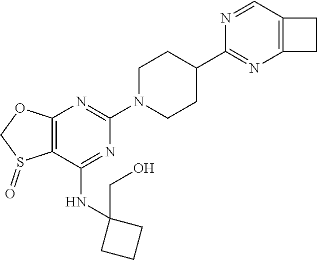 [SMILES: O=S1COc2nc(N3CCC(c4ncc5c(n4)CC5)CC3)nc(NC3(CO)CCC3)c21]

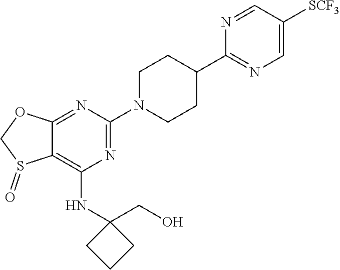

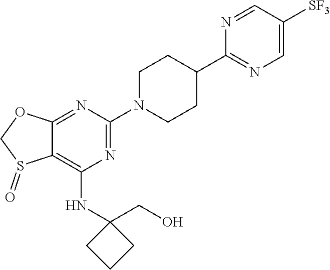

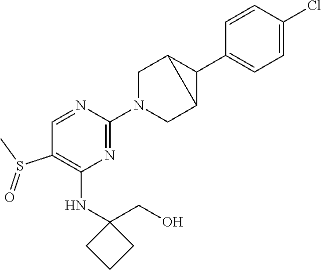 [SMILES: CS(=O)c1cnc(N2CC3C(C2)C3c2ccc(Cl)cc2)nc1NC1(CO)CCC1]

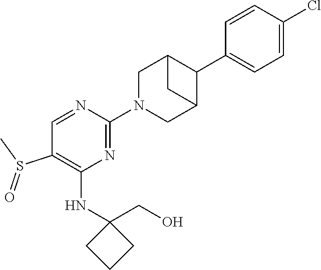 [SMILES: CS(=O)c1cnc(N2CC3CC(C2)C3c2ccc(Cl)cc2)nc1NC1(CO)CCC1]

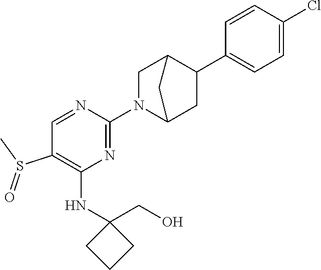 [SMILES: CS(=O)c1cnc(N2CC3CC2CC3c2ccc(Cl)cc2)nc1NC1(CO)CCC1]

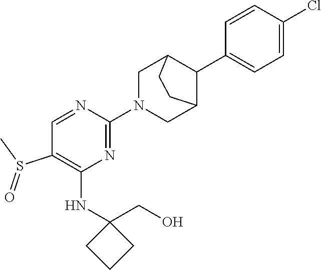 [SMILES: CS(=O)c1cnc(N2CC3CCC(C2)C3c2ccc(Cl)cc2)nc1NC1(CO)CCC1]

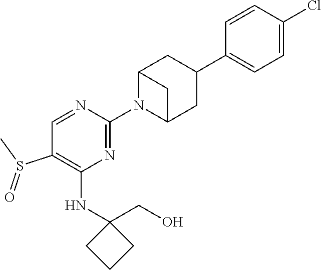 [SMILES: CS(=O)c1cnc(N2C3CC(c4ccc(Cl)cc4)CC2C3)nc1NC1(CO)CCC1]

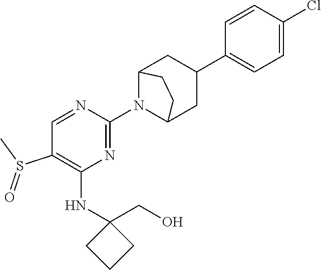 [SMILES: CS(=O)c1cnc(N2C3CCC2CC(c2ccc(Cl)cc2)C3)nc1NC1(CO)CCC1]

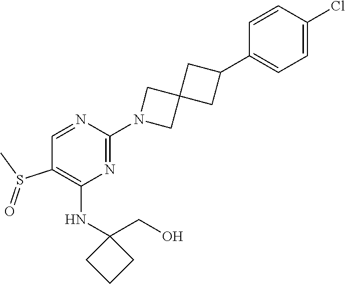 [SMILES: CS(=O)c1cnc(N2CC3(CC(c4ccc(Cl)cc4)C3)C2)nc1NC1(CO)CCC1]

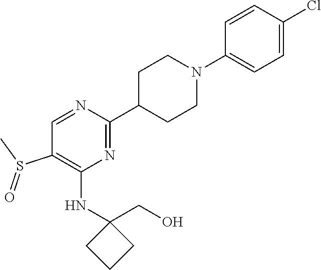 [SMILES: CS(=O)c1cnc(C2CCN(c3ccc(Cl)cc3)CC2)nc1NC1(CO)CCC1]

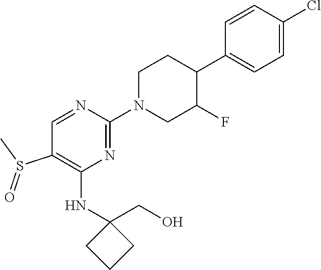 [SMILES: CS(=O)c1cnc(N2CCC(c3ccc(Cl)cc3)C(F)C2)nc1NC1(CO)CCC1]

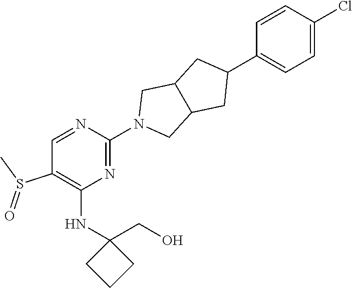 [SMILES: CS(=O)c1cnc(N2CC3CC(c4ccc(Cl)cc4)CC3C2)nc1NC1(CO)CCC1]

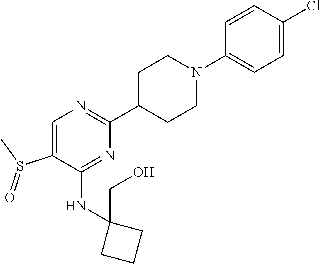 [SMILES: CS(=O)c1cnc(C2CCN(c3ccc(Cl)cc3)CC2)nc1NC1(CO)CCC1]

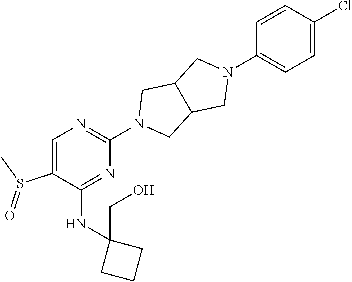 [SMILES: CS(=O)c1cnc(N2CC3CN(c4ccc(Cl)cc4)CC3C2)nc1NC1(CO)CCC1]

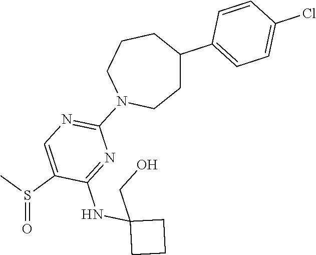 [SMILES: CS(=O)c1cnc(N2CCCC(c3ccc(Cl)cc3)CC2)nc1NC1(CO)CCC1]

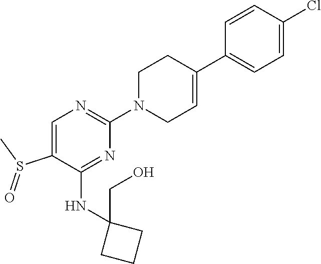 [SMILES: CS(=O)c1cnc(N2CC=C(c3ccc(Cl)cc3)CC2)nc1NC1(CO)CCC1]

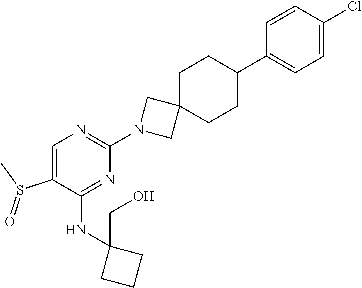 [SMILES: CS(=O)c1cnc(N2CC3(CCC(c4ccc(Cl)cc4)CC3)C2)nc1NC1(CO)CCC1]

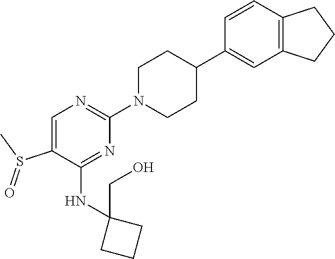 [SMILES: CS(=O)c1cnc(N2CCC(c3ccc4c(c3)CCC4)CC2)nc1NC1(CO)CCC1]

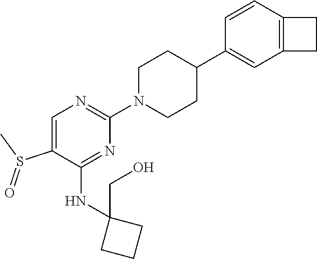 [SMILES: CS(=O)c1cnc(N2CCC(c3ccc4c(c3)CC4)CC2)nc1NC1(CO)CCC1]

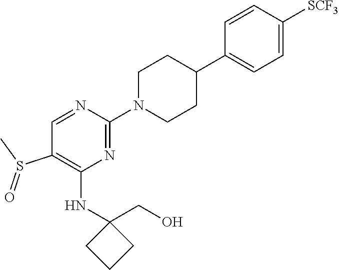

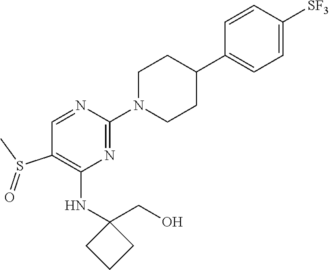

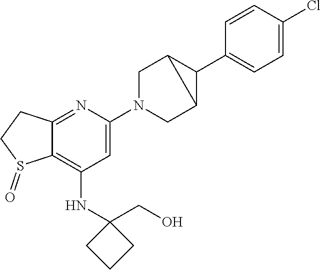 [SMILES: O=S1CCc2nc(N3CC4C(C3)C4c3ccc(Cl)cc3)cc(NC3(CO)CCC3)c21]

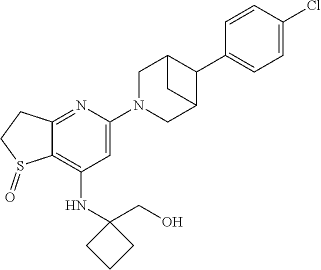 [SMILES: O=S1CCc2nc(N3CC4CC(C3)C4c3ccc(Cl)cc3)cc(NC3(CO)CCC3)c21]

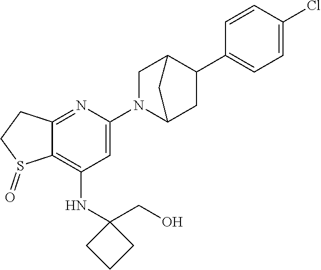 [SMILES: O=S1CCc2nc(N3CC4CC3CC4c3ccc(Cl)cc3)cc(NC3(CO)CCC3)c21]

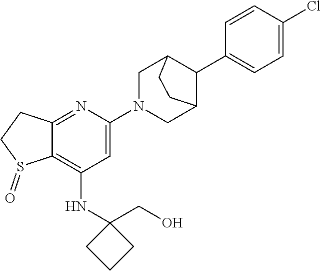 [SMILES: O=S1CCc2nc(N3CC4CCC(C3)C4c3ccc(Cl)cc3)cc(NC3(CO)CCC3)c21]

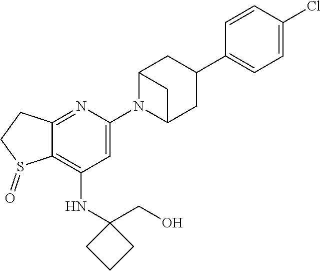 [SMILES: O=S1CCc2nc(N3C4CC(c5ccc(Cl)cc5)CC3C4)cc(NC3(CO)CCC3)c21]

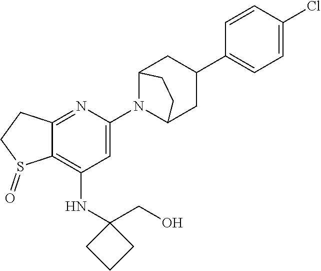 [SMILES: O=S1CCc2nc(N3C4CCC3CC(c3ccc(Cl)cc3)C4)cc(NC3(CO)CCC3)c21]

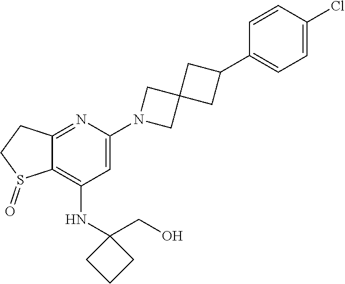 [SMILES: O=S1CCc2nc(N3CC4(CC(c5ccc(Cl)cc5)C4)C3)cc(NC3(CO)CCC3)c21]

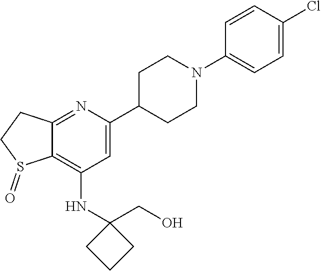 [SMILES: O=S1CCc2nc(C3CCN(c4ccc(Cl)cc4)CC3)cc(NC3(CO)CCC3)c21]

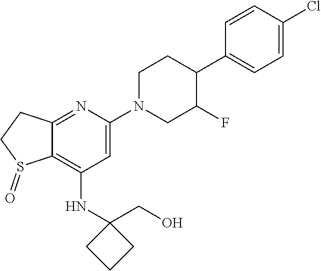 [SMILES: O=S1CCc2nc(N3CCC(c4ccc(Cl)cc4)C(F)C3)cc(NC3(CO)CCC3)c21]

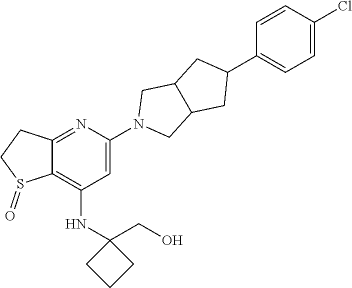 [SMILES: O=S1CCc2nc(N3CC4CC(c5ccc(Cl)cc5)CC4C3)cc(NC3(CO)CCC3)c21]

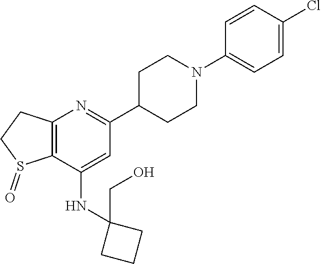 [SMILES: O=S1CCc2nc(C3CCN(c4ccc(Cl)cc4)CC3)cc(NC3(CO)CCC3)c21]

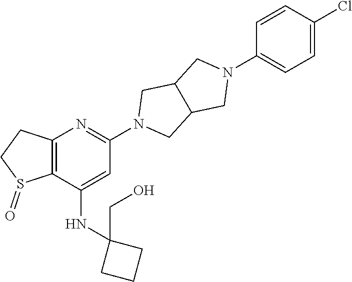 [SMILES: O=S1CCc2nc(N3CC4CN(c5ccc(Cl)cc5)CC4C3)cc(NC3(CO)CCC3)c21]

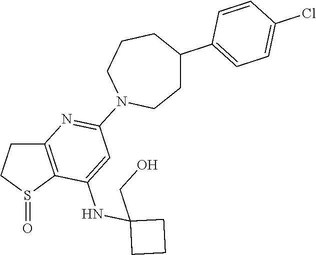 [SMILES: O=S1CCc2nc(N3CCCC(c4ccc(Cl)cc4)CC3)cc(NC3(CO)CCC3)c21]

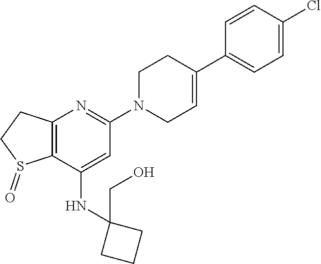 [SMILES: O=S1CCc2nc(N3CC=C(c4ccc(Cl)cc4)CC3)cc(NC3(CO)CCC3)c21]

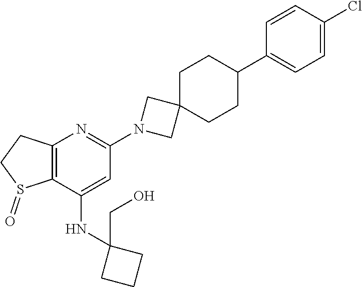 [SMILES: O=S1CCc2nc(N3CC4(CCC(c5ccc(Cl)cc5)CC4)C3)cc(NC3(CO)CCC3)c21]

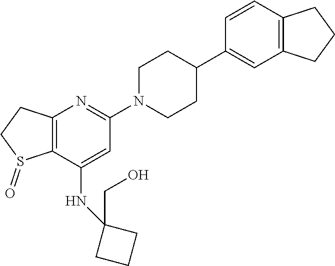 [SMILES: O=S1CCc2nc(N3CCC(c4ccc5c(c4)CCC5)CC3)cc(NC3(CO)CCC3)c21]

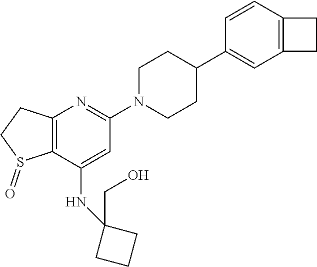 [SMILES: O=S1CCc2nc(N3CCC(c4ccc5c(c4)CC5)CC3)cc(NC3(CO)CCC3)c21]

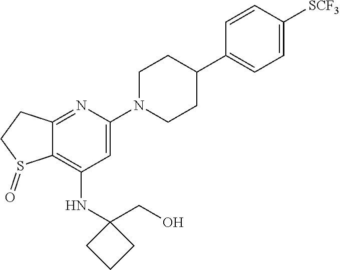

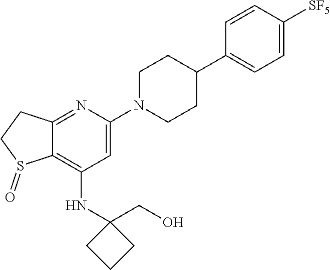

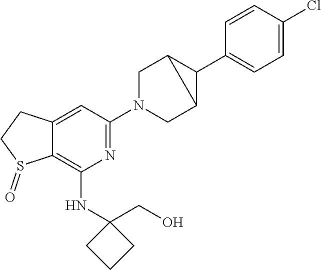 [SMILES: O=S1CCc2cc(N3CC4C(C3)C4c3ccc(Cl)cc3)nc(NC3(CO)CCC3)c21]

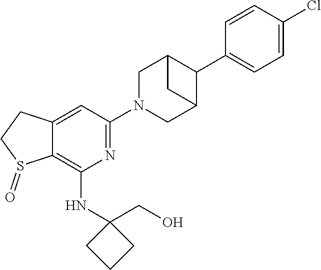 [SMILES: O=S1CCc2cc(N3CC4CC(C3)C4c3ccc(Cl)cc3)nc(NC3(CO)CCC3)c21]

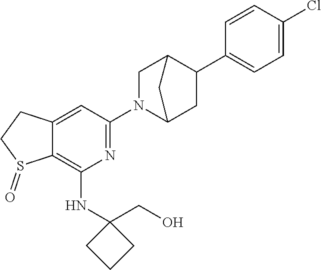 [SMILES: O=S1CCc2cc(N3CC4CC3CC4c3ccc(Cl)cc3)nc(NC3(CO)CCC3)c21]

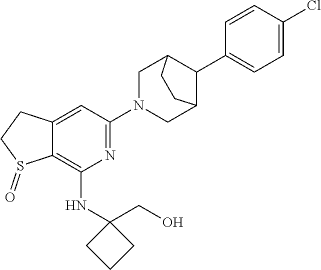 [SMILES: O=S1CCc2cc(N3CC4CCC(C3)C4c3ccc(Cl)cc3)nc(NC3(CO)CCC3)c21]

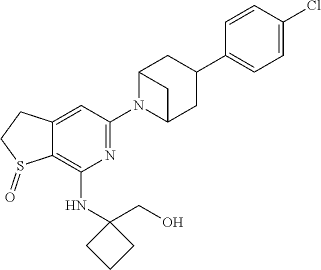 [SMILES: O=S1CCc2cc(N3C4CC(c5ccc(Cl)cc5)CC3C4)nc(NC3(CO)CCC3)c21]

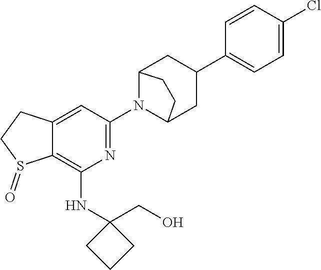 [SMILES: O=S1CCc2cc(N3C4CCC3CC(c3ccc(Cl)cc3)C4)nc(NC3(CO)CCC3)c21]

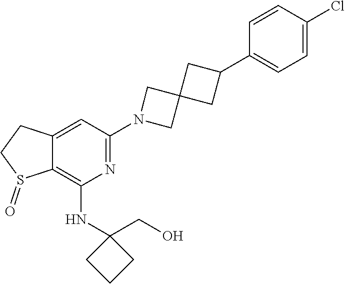 [SMILES: O=S1CCc2cc(N3CC4(CC(c5ccc(Cl)cc5)C4)C3)nc(NC3(CO)CCC3)c21]

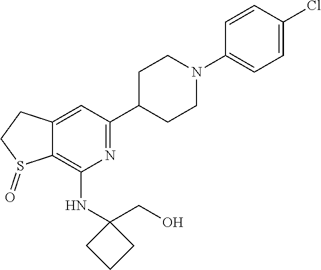 [SMILES: O=S1CCc2cc(C3CCN(c4ccc(Cl)cc4)CC3)nc(NC3(CO)CCC3)c21]

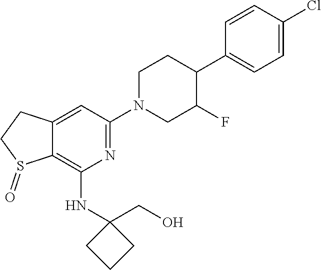 [SMILES: O=S1CCc2cc(N3CCC(c4ccc(Cl)cc4)C(F)C3)nc(NC3(CO)CCC3)c21]

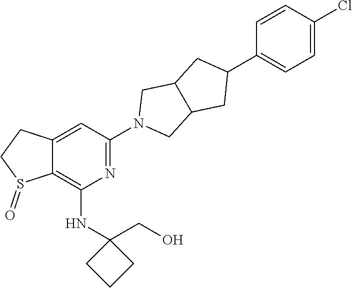 [SMILES: O=S1CCc2cc(N3CC4CC(c5ccc(Cl)cc5)CC4C3)nc(NC3(CO)CCC3)c21]

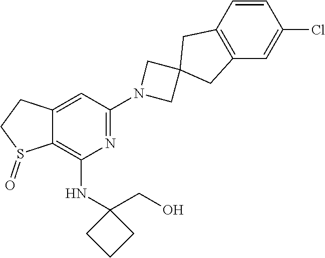 [SMILES: O=S1CCc2cc(N3CC4(Cc5ccc(Cl)cc5C4)C3)nc(NC3(CO)CCC3)c21]

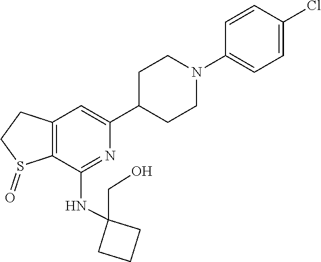 [SMILES: O=S1CCc2cc(C3CCN(c4ccc(Cl)cc4)CC3)nc(NC3(CO)CCC3)c21]

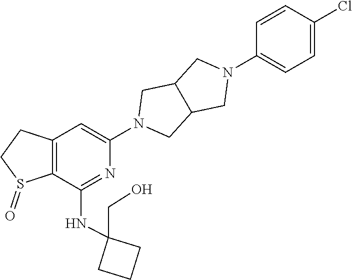 [SMILES: O=S1CCc2cc(N3CC4CN(c5ccc(Cl)cc5)CC4C3)nc(NC3(CO)CCC3)c21]

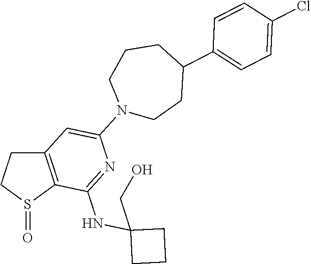 [SMILES: O=S1CCc2cc(N3CCCC(c4ccc(Cl)cc4)CC3)nc(NC3(CO)CCC3)c21]

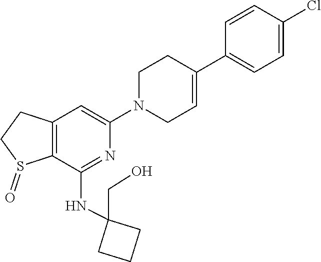 [SMILES: O=S1CCc2cc(N3CC=C(c4ccc(Cl)cc4)CC3)nc(NC3(CO)CCC3)c21]

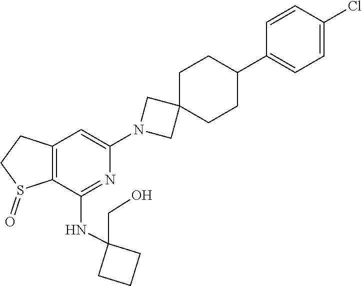 [SMILES: O=S1CCc2cc(N3CC4(CCC(c5ccc(Cl)cc5)CC4)C3)nc(NC3(CO)CCC3)c21]

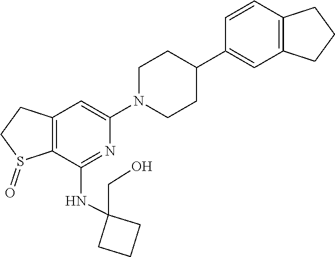 [SMILES: O=S1CCc2cc(N3CCC(c4ccc5c(c4)CCC5)CC3)nc(NC3(CO)CCC3)c21]

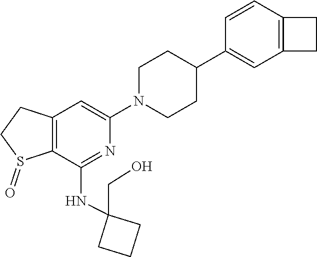 [SMILES: O=S1CCc2cc(N3CCC(c4ccc5c(c4)CC5)CC3)nc(NC3(CO)CCC3)c21]

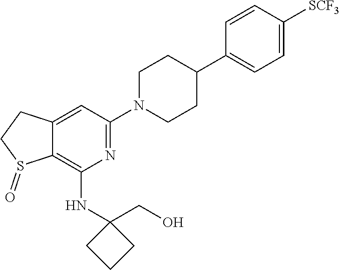

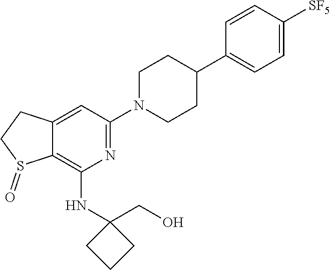

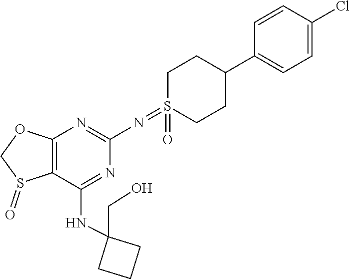 [SMILES: O=S1COc2nc(N=S3(=O)CCC(c4ccc(Cl)cc4)CC3)nc(NC3(CO)CCC3)c21]

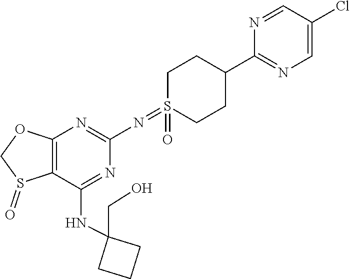 [SMILES: O=S1COc2nc(N=S3(=O)CCC(c4ncc(Cl)cn4)CC3)nc(NC3(CO)CCC3)c21]

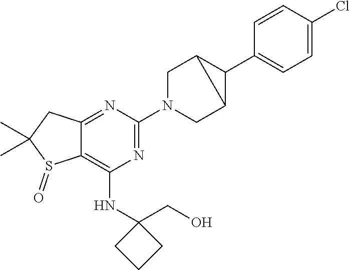 [SMILES: CC1(C)Cc2nc(N3CC4C(C3)C4c3ccc(Cl)cc3)nc(NC3(CO)CCC3)c2S1=O]

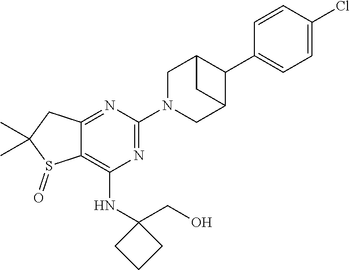 [SMILES: CC1(C)Cc2nc(N3CC4CC(C3)C4c3ccc(Cl)cc3)nc(NC3(CO)CCC3)c2S1=O]

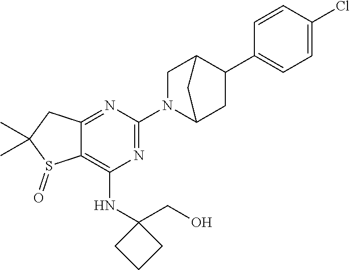 [SMILES: CC1(C)Cc2nc(N3CC4CC3CC4c3ccc(Cl)cc3)nc(NC3(CO)CCC3)c2S1=O]

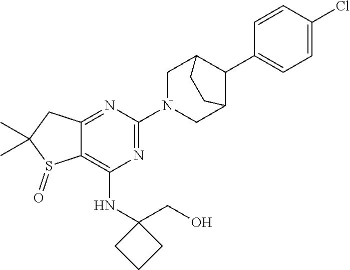 [SMILES: CC1(C)Cc2nc(N3CC4CCC(C3)C4c3ccc(Cl)cc3)nc(NC3(CO)CCC3)c2S1=O]

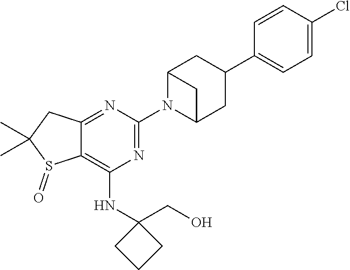 [SMILES: CC1(C)Cc2nc(N3C4CC(c5ccc(Cl)cc5)CC3C4)nc(NC3(CO)CCC3)c2S1=O]

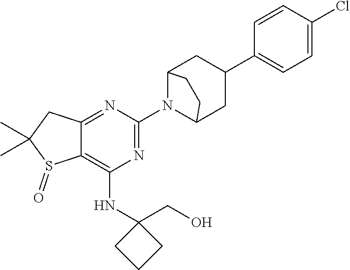 [SMILES: CC1(C)Cc2nc(N3C4CCC3CC(c3ccc(Cl)cc3)C4)nc(NC3(CO)CCC3)c2S1=O]

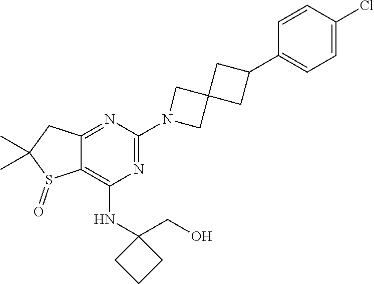 [SMILES: CC1(C)Cc2nc(N3CC4(CC(c5ccc(Cl)cc5)C4)C3)nc(NC3(CO)CCC3)c2S1=O]

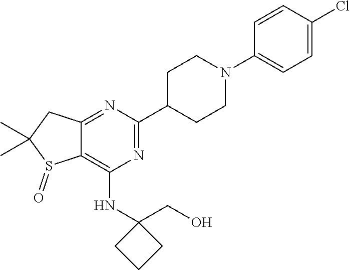 [SMILES: CC1(C)Cc2nc(C3CCN(c4ccc(Cl)cc4)CC3)nc(NC3(CO)CCC3)c2S1=O]

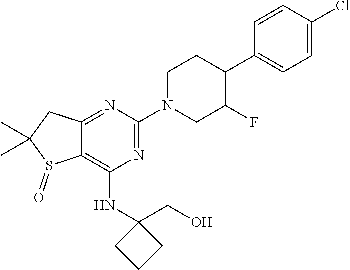 [SMILES: CC1(C)Cc2nc(N3CCC(c4ccc(Cl)cc4)C(F)C3)nc(NC3(CO)CCC3)c2S1=O]

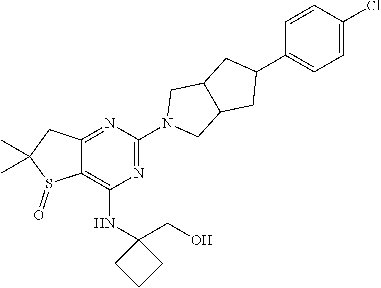 [SMILES: CC1(C)Cc2nc(N3CC4CC(c5ccc(Cl)cc5)CC4C3)nc(NC3(CO)CCC3)c2S1=O]

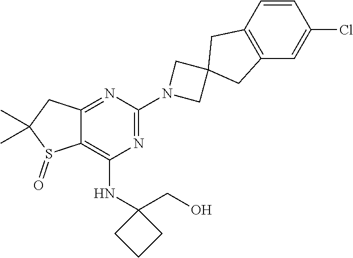 [SMILES: CC1(C)Cc2nc(N3CC4(Cc5ccc(Cl)cc5C4)C3)nc(NC3(CO)CCC3)c2S1=O]

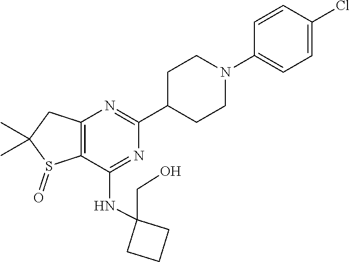 [SMILES: CC1(C)Cc2nc(C3CCN(c4ccc(Cl)cc4)CC3)nc(NC3(CO)CCC3)c2S1=O]

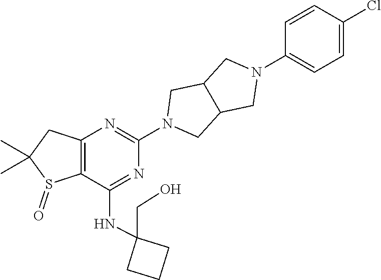 [SMILES: CC1(C)Cc2nc(N3CC4CN(c5ccc(Cl)cc5)CC4C3)nc(NC3(CO)CCC3)c2S1=O]

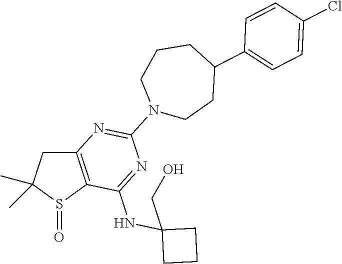 [SMILES: CC1(C)Cc2nc(N3CCCC(c4ccc(Cl)cc4)CC3)nc(NC3(CO)CCC3)c2S1=O]

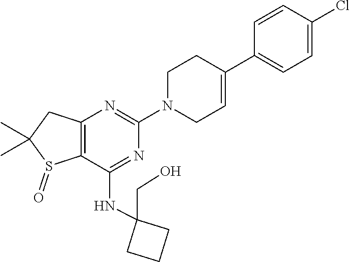 [SMILES: CC1(C)Cc2nc(N3CC=C(c4ccc(Cl)cc4)CC3)nc(NC3(CO)CCC3)c2S1=O]

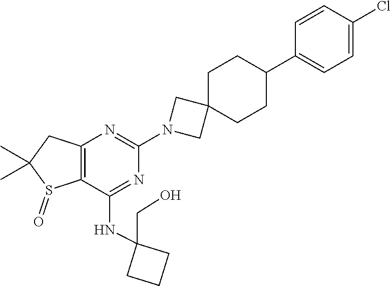 [SMILES: CC1(C)Cc2nc(N3CC4(CCC(c5ccc(Cl)cc5)CC4)C3)nc(NC3(CO)CCC3)c2S1=O]

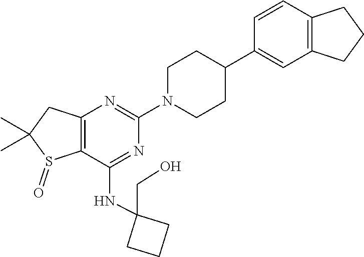 [SMILES: CC1(C)Cc2nc(N3CCC(c4ccc5c(c4)CCC5)CC3)nc(NC3(CO)CCC3)c2S1=O]

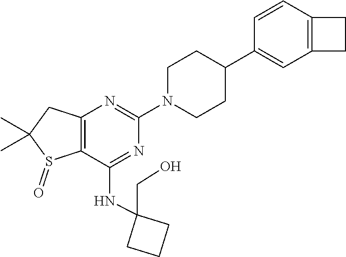 [SMILES: CC1(C)Cc2nc(N3CCC(c4ccc5c(c4)CC5)CC3)nc(NC3(CO)CCC3)c2S1=O]

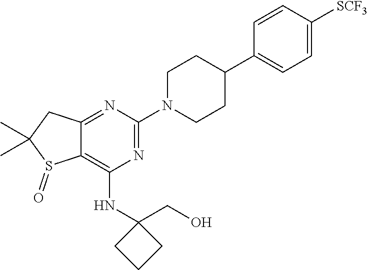

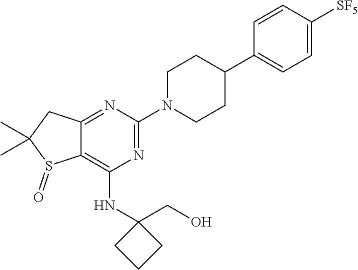

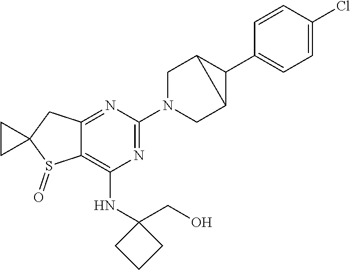 [SMILES: O=S1c2c(nc(N3CC4C(C3)C4c3ccc(Cl)cc3)nc2NC2(CO)CCC2)CC12CC2]

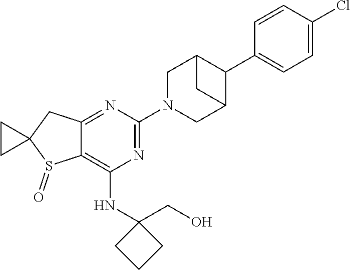 [SMILES: O=S1c2c(nc(N3CC4CC(C3)C4c3ccc(Cl)cc3)nc2NC2(CO)CCC2)CC12CC2]

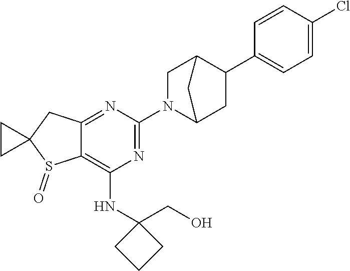 [SMILES: O=S1c2c(nc(N3CC4CC3CC4c3ccc(Cl)cc3)nc2NC2(CO)CCC2)CC12CC2]

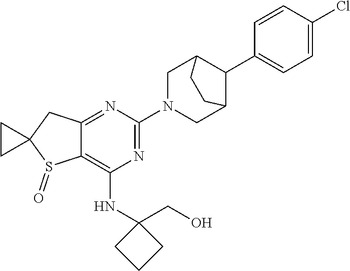 [SMILES: O=S1c2c(nc(N3CC4CCC(C3)C4c3ccc(Cl)cc3)nc2NC2(CO)CCC2)CC12CC2]

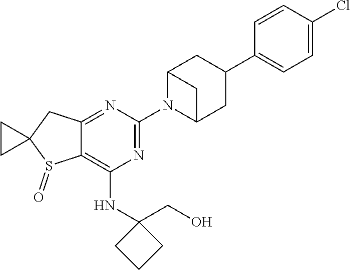 [SMILES: O=S1c2c(nc(N3C4CC(c5ccc(Cl)cc5)CC3C4)nc2NC2(CO)CCC2)CC12CC2]

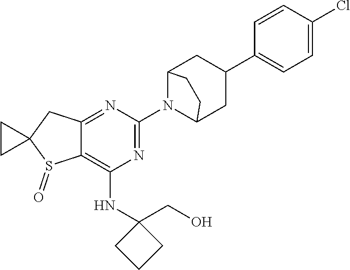 [SMILES: O=S1c2c(nc(N3C4CCC3CC(c3ccc(Cl)cc3)C4)nc2NC2(CO)CCC2)CC12CC2]

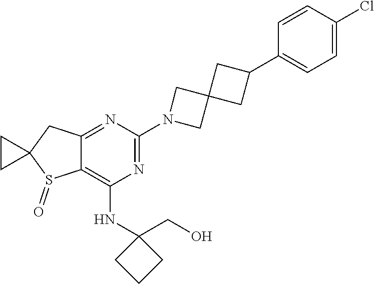 [SMILES: O=S1c2c(nc(N3CC4(CC(c5ccc(Cl)cc5)C4)C3)nc2NC2(CO)CCC2)CC12CC2]

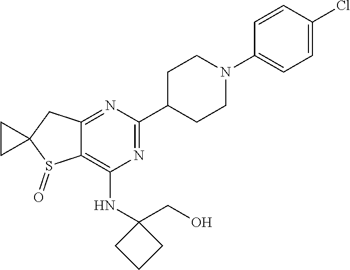 [SMILES: O=S1c2c(nc(C3CCN(c4ccc(Cl)cc4)CC3)nc2NC2(CO)CCC2)CC12CC2]

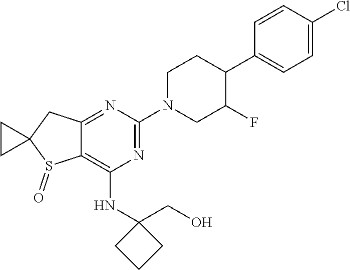 [SMILES: O=S1c2c(nc(N3CCC(c4ccc(Cl)cc4)C(F)C3)nc2NC2(CO)CCC2)CC12CC2]

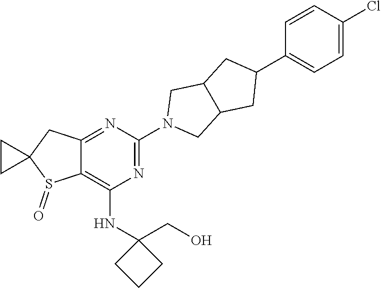 [SMILES: O=S1c2c(nc(N3CC4CC(c5ccc(Cl)cc5)CC4C3)nc2NC2(CO)CCC2)CC12CC2]

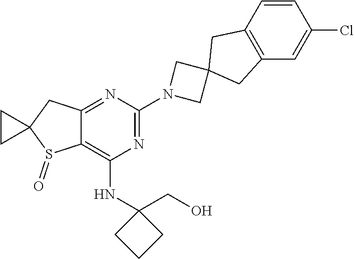 [SMILES: O=S1c2c(nc(N3CC4(Cc5ccc(Cl)cc5C4)C3)nc2NC2(CO)CCC2)CC12CC2]

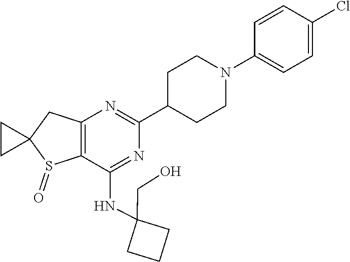 [SMILES: O=S1c2c(nc(C3CCN(c4ccc(Cl)cc4)CC3)nc2NC2(CO)CCC2)CC12CC2]

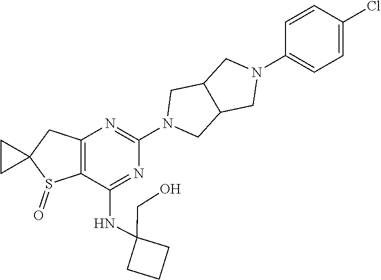 [SMILES: O=S1c2c(nc(N3CC4CN(c5ccc(Cl)cc5)CC4C3)nc2NC2(CO)CCC2)CC12CC2]

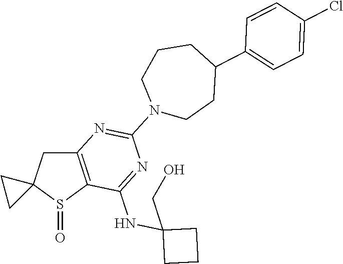 [SMILES: O=S1c2c(nc(N3CCCC(c4ccc(Cl)cc4)CC3)nc2NC2(CO)CCC2)CC12CC2]

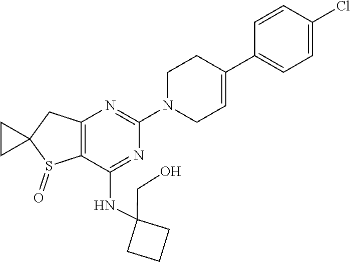 [SMILES: O=S1c2c(nc(N3CC=C(c4ccc(Cl)cc4)CC3)nc2NC2(CO)CCC2)CC12CC2]

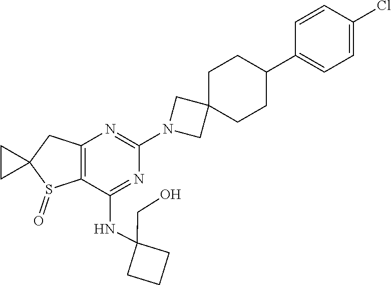 [SMILES: O=S1c2c(nc(N3CC4(CCC(c5ccc(Cl)cc5)CC4)C3)nc2NC2(CO)CCC2)CC12CC2]

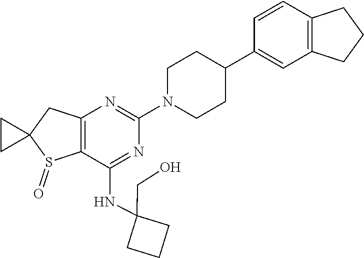 [SMILES: O=S1c2c(nc(N3CCC(c4ccc5c(c4)CCC5)CC3)nc2NC2(CO)CCC2)CC12CC2]

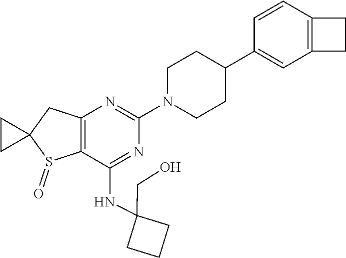 [SMILES: O=S1c2c(nc(N3CCC(c4ccc5c(c4)CC5)CC3)nc2NC2(CO)CCC2)CC12CC2]

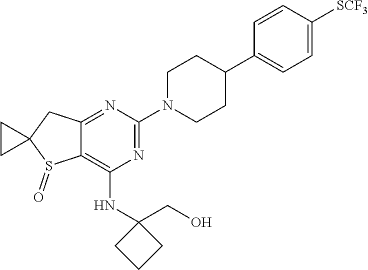

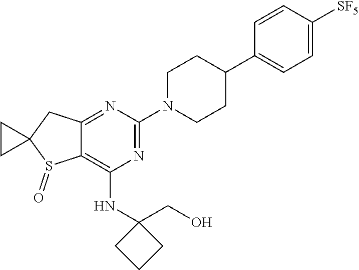

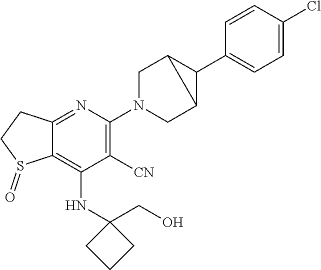 [SMILES: N#Cc1c(N2CC3C(C2)C3c2ccc(Cl)cc2)nc2c(c1NC1(CO)CCC1)S(=O)CC2]

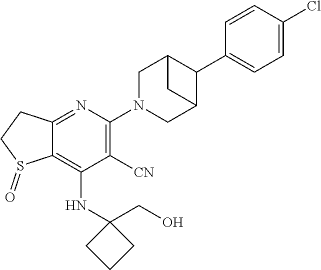 [SMILES: N#Cc1c(N2CC3CC(C2)C3c2ccc(Cl)cc2)nc2c(c1NC1(CO)CCC1)S(=O)CC2]

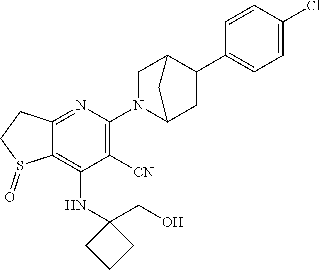 [SMILES: N#Cc1c(N2CC3CC2CC3c2ccc(Cl)cc2)nc2c(c1NC1(CO)CCC1)S(=O)CC2]

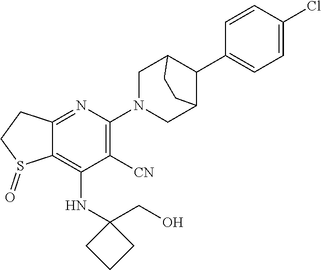 [SMILES: N#Cc1c(N2CC3CCC(C2)C3c2ccc(Cl)cc2)nc2c(c1NC1(CO)CCC1)S(=O)CC2]

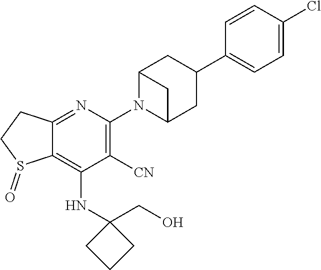 [SMILES: N#Cc1c(N2C3CC(c4ccc(Cl)cc4)CC2C3)nc2c(c1NC1(CO)CCC1)S(=O)CC2]

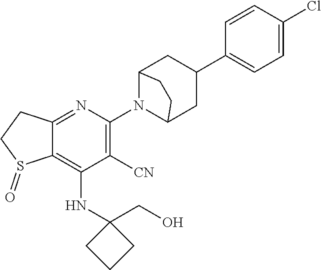 [SMILES: N#Cc1c(N2C3CCC2CC(c2ccc(Cl)cc2)C3)nc2c(c1NC1(CO)CCC1)S(=O)CC2]

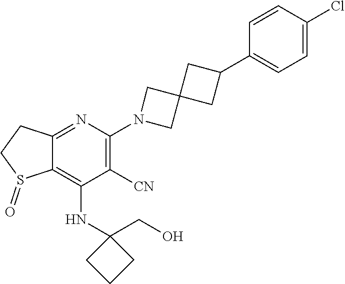 [SMILES: N#Cc1c(N2CC3(CC(c4ccc(Cl)cc4)C3)C2)nc2c(c1NC1(CO)CCC1)S(=O)CC2]

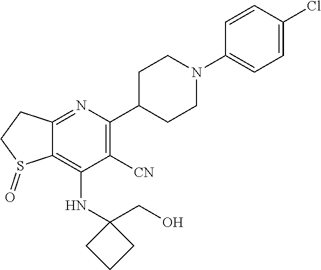 [SMILES: N#Cc1c(C2CCN(c3ccc(Cl)cc3)CC2)nc2c(c1NC1(CO)CCC1)S(=O)CC2]

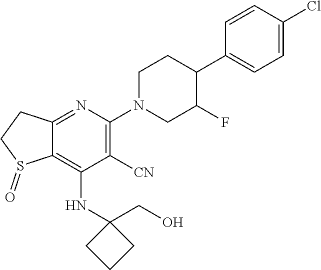 [SMILES: N#Cc1c(N2CCC(c3ccc(Cl)cc3)C(F)C2)nc2c(c1NC1(CO)CCC1)S(=O)CC2]

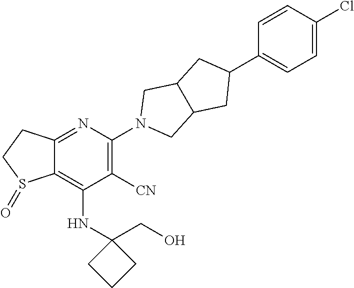 [SMILES: N#Cc1c(N2CC3CC(c4ccc(Cl)cc4)CC3C2)nc2c(c1NC1(CO)CCC1)S(=O)CC2]

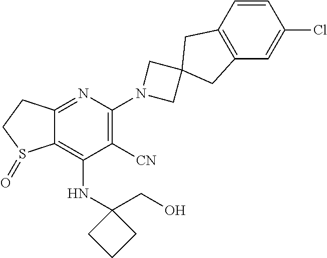 [SMILES: N#Cc1c(N2CC3(Cc4ccc(Cl)cc4C3)C2)nc2c(c1NC1(CO)CCC1)S(=O)CC2]

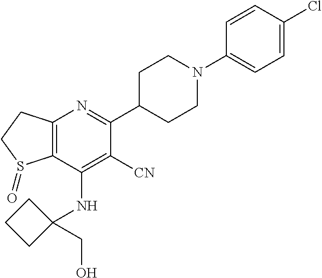 [SMILES: N#Cc1c(C2CCN(c3ccc(Cl)cc3)CC2)nc2c(c1NC1(CO)CCC1)S(=O)CC2]

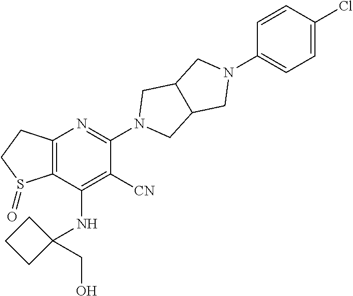 [SMILES: N#Cc1c(N2CC3CN(c4ccc(Cl)cc4)CC3C2)nc2c(c1NC1(CO)CCC1)S(=O)CC2]

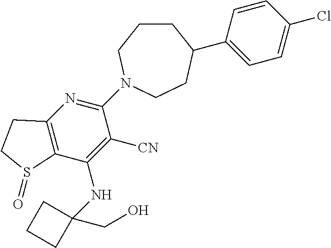 [SMILES: N#Cc1c(N2CCCC(c3ccc(Cl)cc3)CC2)nc2c(c1NC1(CO)CCC1)S(=O)CC2]

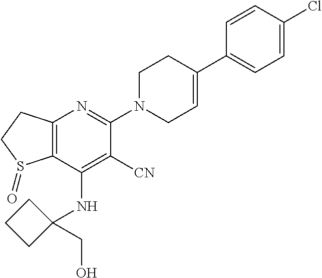 [SMILES: N#Cc1c(N2CC=C(c3ccc(Cl)cc3)CC2)nc2c(c1NC1(CO)CCC1)S(=O)CC2]

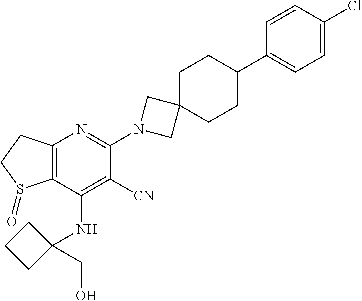 [SMILES: N#Cc1c(N2CC3(CCC(c4ccc(Cl)cc4)CC3)C2)nc2c(c1NC1(CO)CCC1)S(=O)CC2]

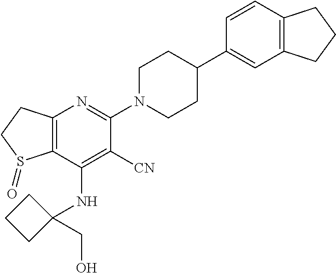 [SMILES: N#Cc1c(N2CCC(c3ccc4c(c3)CCC4)CC2)nc2c(c1NC1(CO)CCC1)S(=O)CC2]

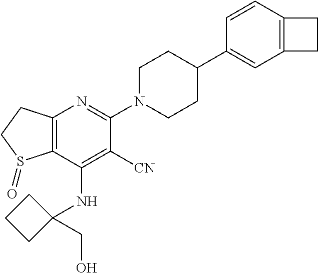 [SMILES: N#Cc1c(N2CCC(c3ccc4c(c3)CC4)CC2)nc2c(c1NC1(CO)CCC1)S(=O)CC2]

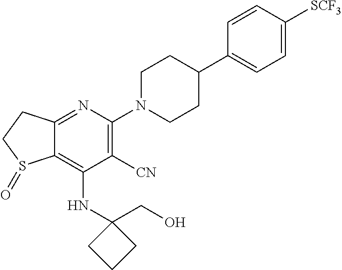

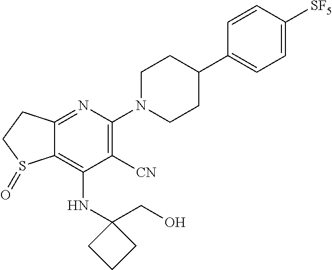

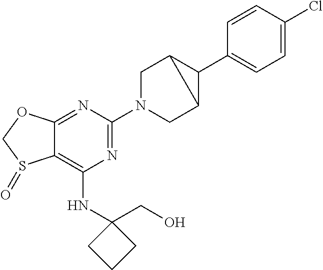 [SMILES: O=S1COc2nc(N3CC4C(C3)C4c3ccc(Cl)cc3)nc(NC3(CO)CCC3)c21]

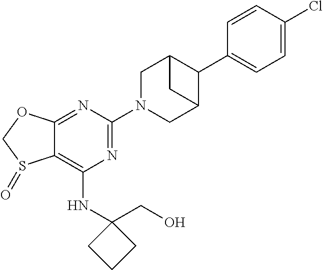 [SMILES: O=S1COc2nc(N3CC4CC(C3)C4c3ccc(Cl)cc3)nc(NC3(CO)CCC3)c21]

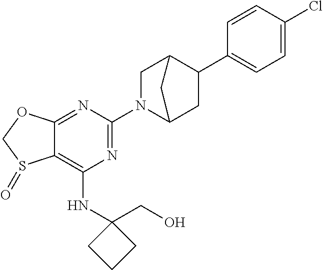 [SMILES: O=S1COc2nc(N3CC4CC3CC4c3ccc(Cl)cc3)nc(NC3(CO)CCC3)c21]

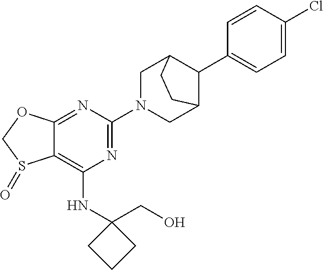 [SMILES: O=S1COc2nc(N3CC4CCC(C3)C4c3ccc(Cl)cc3)nc(NC3(CO)CCC3)c21]

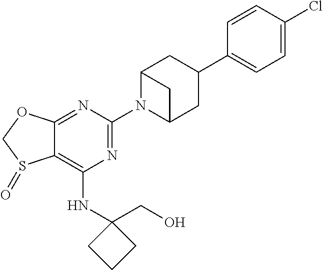 [SMILES: O=S1COc2nc(N3C4CC(c5ccc(Cl)cc5)CC3C4)nc(NC3(CO)CCC3)c21]

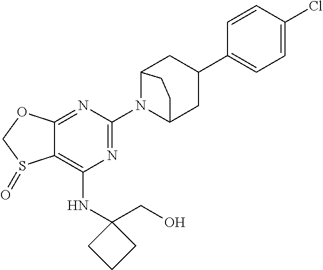 [SMILES: O=S1COc2nc(N3C4CCC3CC(c3ccc(Cl)cc3)C4)nc(NC3(CO)CCC3)c21]

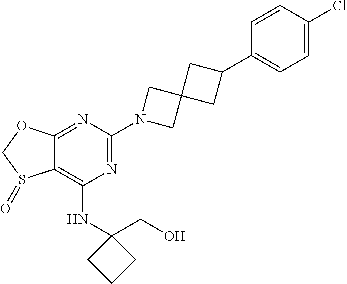 [SMILES: O=S1COc2nc(N3CC4(CC(c5ccc(Cl)cc5)C4)C3)nc(NC3(CO)CCC3)c21]

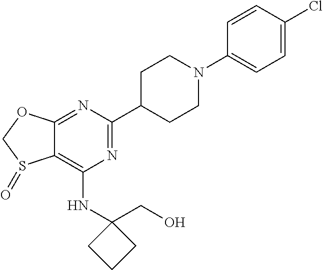 [SMILES: O=S1COc2nc(C3CCN(c4ccc(Cl)cc4)CC3)nc(NC3(CO)CCC3)c21]

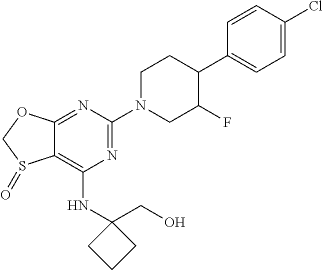 [SMILES: O=S1COc2nc(N3CCC(c4ccc(Cl)cc4)C(F)C3)nc(NC3(CO)CCC3)c21]

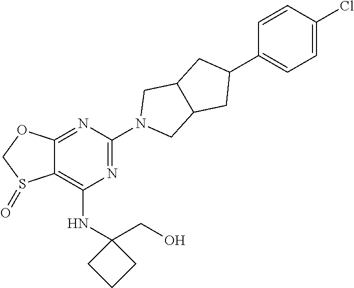 [SMILES: O=S1COc2nc(N3CC4CC(c5ccc(Cl)cc5)CC4C3)nc(NC3(CO)CCC3)c21]

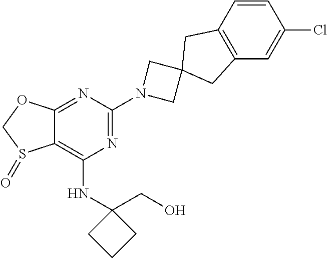 [SMILES: O=S1COc2nc(N3CC4(Cc5ccc(Cl)cc5C4)C3)nc(NC3(CO)CCC3)c21]

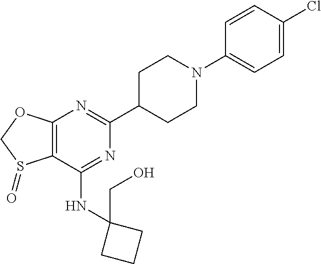 [SMILES: O=S1COc2nc(C3CCN(c4ccc(Cl)cc4)CC3)nc(NC3(CO)CCC3)c21]

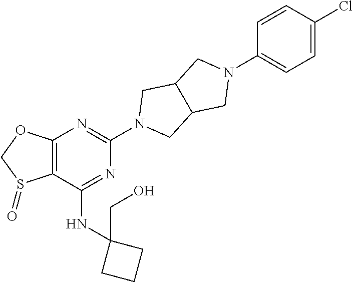 [SMILES: O=S1COc2nc(N3CC4CN(c5ccc(Cl)cc5)CC4C3)nc(NC3(CO)CCC3)c21]

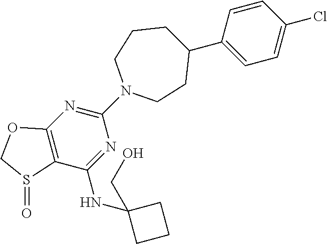 [SMILES: O=S1COc2nc(N3CCCC(c4ccc(Cl)cc4)CC3)nc(NC3(CO)CCC3)c21]

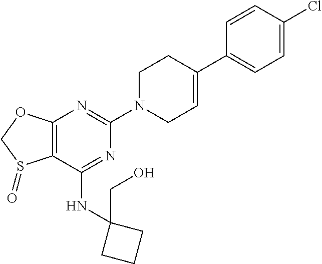 [SMILES: O=S1COc2nc(N3CC=C(c4ccc(Cl)cc4)CC3)nc(NC3(CO)CCC3)c21]

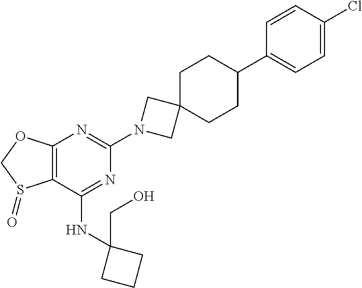 [SMILES: O=S1COc2nc(N3CC4(CCC(c5ccc(Cl)cc5)CC4)C3)nc(NC3(CO)CCC3)c21]

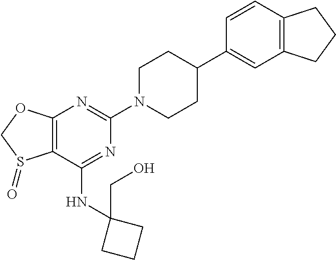 [SMILES: O=S1COc2nc(N3CCC(c4ccc5c(c4)CCC5)CC3)nc(NC3(CO)CCC3)c21]

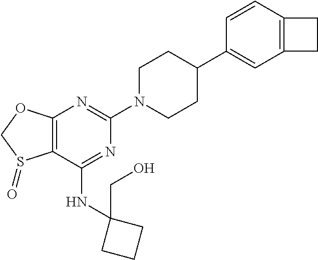 [SMILES: O=S1COc2nc(N3CCC(c4ccc5c(c4)CC5)CC3)nc(NC3(CO)CCC3)c21]

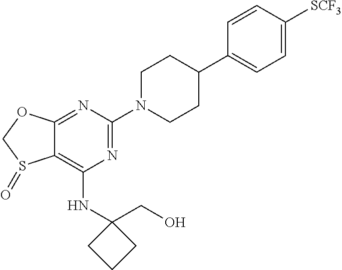

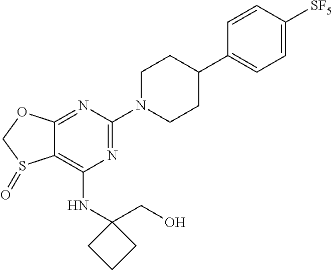

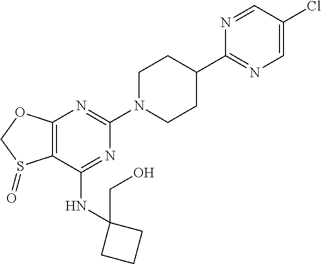 [SMILES: O=S1COc2nc(N3CCC(c4ncc(Cl)cn4)CC3)nc(NC3(CO)CCC3)c21]

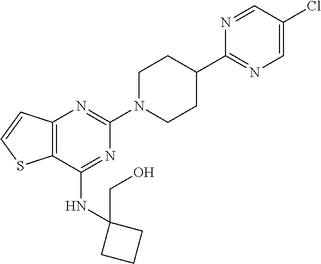 [SMILES: OCC1(Nc2nc(N3CCC(c4ncc(Cl)cn4)CC3)nc3ccsc23)CCC1]

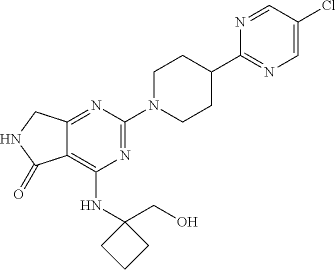 [SMILES: O=C1NCc2nc(N3CCC(c4ncc(Cl)cn4)CC3)nc(NC3(CO)CCC3)c21]

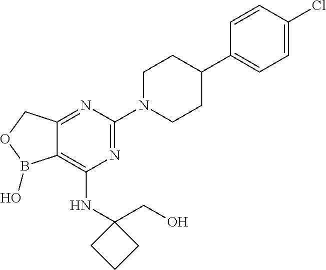 [SMILES: OCC1(Nc2nc(N3CCC(c4ccc(Cl)cc4)CC3)nc3c2B(O)OC3)CCC1]

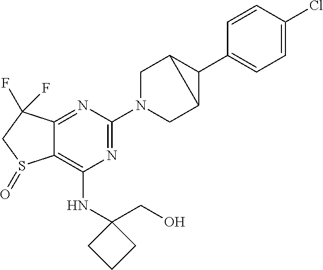 [SMILES: O=S1CC(F)(F)c2nc(N3CC4C(C3)C4c3ccc(Cl)cc3)nc(NC3(CO)CCC3)c21]

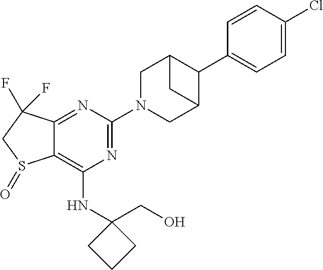 [SMILES: O=S1CC(F)(F)c2nc(N3CC4CC(C3)C4c3ccc(Cl)cc3)nc(NC3(CO)CCC3)c21]

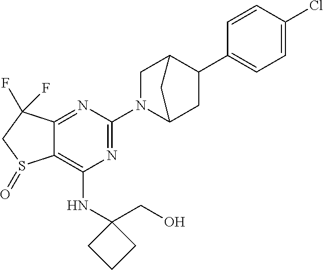 [SMILES: O=S1CC(F)(F)c2nc(N3CC4CC3CC4c3ccc(Cl)cc3)nc(NC3(CO)CCC3)c21]

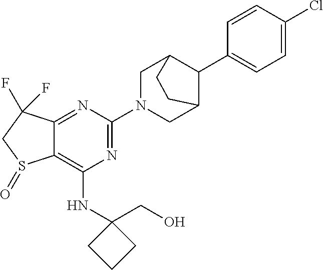 [SMILES: O=S1CC(F)(F)c2nc(N3CC4CCC(C3)C4c3ccc(Cl)cc3)nc(NC3(CO)CCC3)c21]

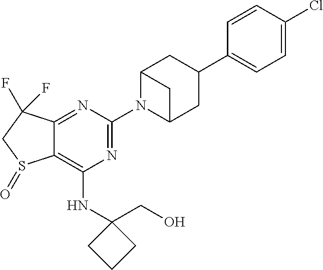 [SMILES: O=S1CC(F)(F)c2nc(N3C4CC(c5ccc(Cl)cc5)CC3C4)nc(NC3(CO)CCC3)c21]

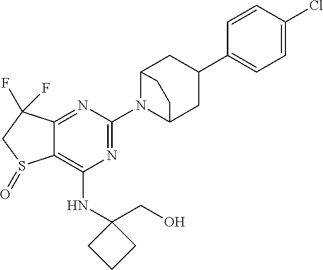 [SMILES: O=S1CC(F)(F)c2nc(N3C4CCC3CC(c3ccc(Cl)cc3)C4)nc(NC3(CO)CCC3)c21]

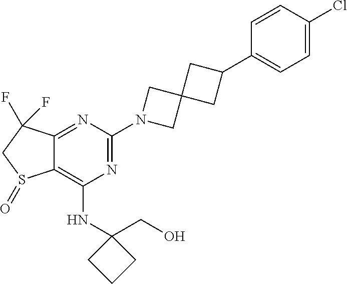 [SMILES: O=S1CC(F)(F)c2nc(N3CC4(CC(c5ccc(Cl)cc5)C4)C3)nc(NC3(CO)CCC3)c21]

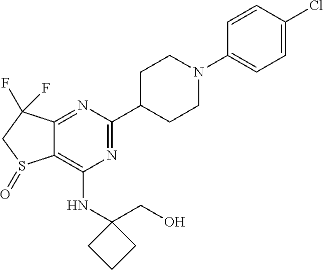 [SMILES: O=S1CC(F)(F)c2nc(C3CCN(c4ccc(Cl)cc4)CC3)nc(NC3(CO)CCC3)c21]

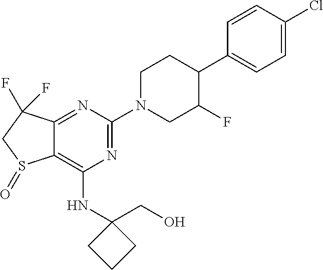 [SMILES: O=S1CC(F)(F)c2nc(N3CCC(c4ccc(Cl)cc4)C(F)C3)nc(NC3(CO)CCC3)c21]

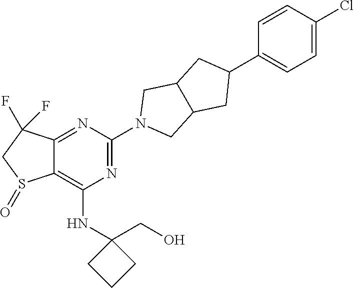 [SMILES: O=S1CC(F)(F)c2nc(N3CC4CC(c5ccc(Cl)cc5)CC4C3)nc(NC3(CO)CCC3)c21]

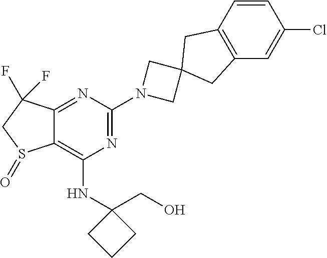 [SMILES: O=S1CC(F)(F)c2nc(N3CC4(Cc5ccc(Cl)cc5C4)C3)nc(NC3(CO)CCC3)c21]

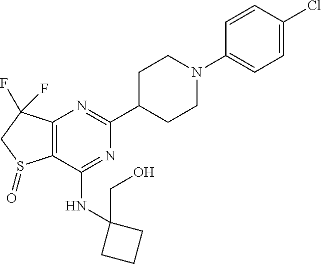 [SMILES: O=S1CC(F)(F)c2nc(C3CCN(c4ccc(Cl)cc4)CC3)nc(NC3(CO)CCC3)c21]

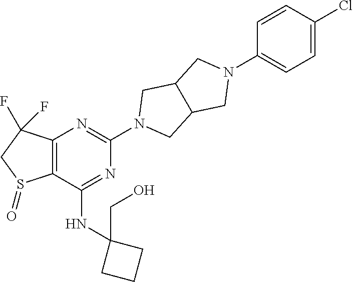 [SMILES: O=S1CC(F)(F)c2nc(N3CC4CN(c5ccc(Cl)cc5)CC4C3)nc(NC3(CO)CCC3)c21]

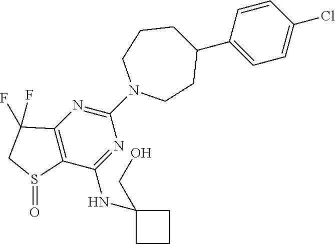 [SMILES: O=S1CC(F)(F)c2nc(N3CCCC(c4ccc(Cl)cc4)CC3)nc(NC3(CO)CCC3)c21]

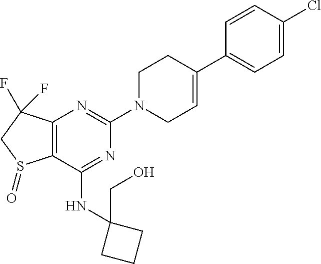 [SMILES: O=S1CC(F)(F)c2nc(N3CC=C(c4ccc(Cl)cc4)CC3)nc(NC3(CO)CCC3)c21]

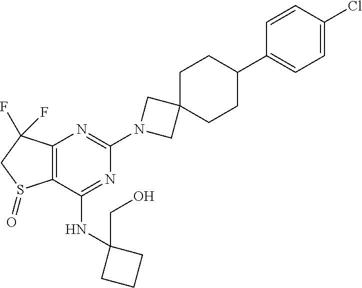 [SMILES: O=S1CC(F)(F)c2nc(N3CC4(CCC(c5ccc(Cl)cc5)CC4)C3)nc(NC3(CO)CCC3)c21]

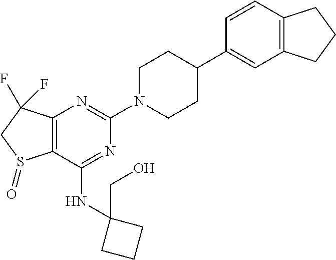 [SMILES: O=S1CC(F)(F)c2nc(N3CCC(c4ccc5c(c4)CCC5)CC3)nc(NC3(CO)CCC3)c21]

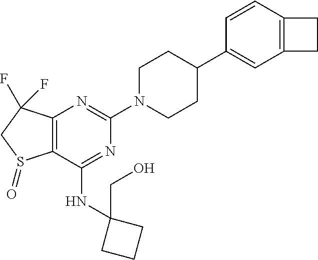 [SMILES: O=S1CC(F)(F)c2nc(N3CCC(c4ccc5c(c4)CC5)CC3)nc(NC3(CO)CCC3)c21]

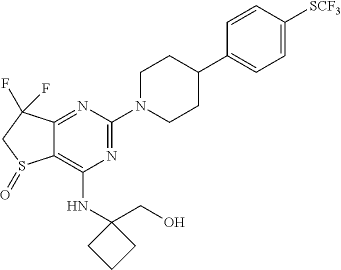

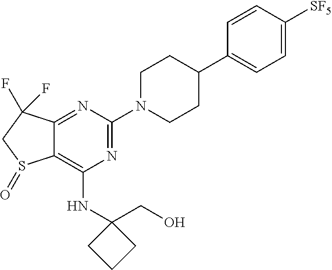

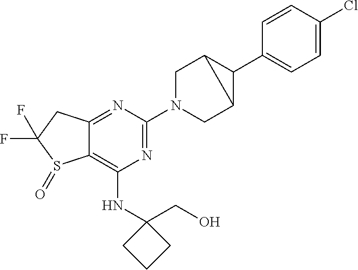 [SMILES: O=S1c2c(nc(N3CC4C(C3)C4c3ccc(Cl)cc3)nc2NC2(CO)CCC2)CC1(F)F]

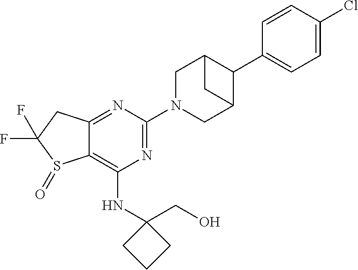 [SMILES: O=S1c2c(nc(N3CC4CC(C3)C4c3ccc(Cl)cc3)nc2NC2(CO)CCC2)CC1(F)F]

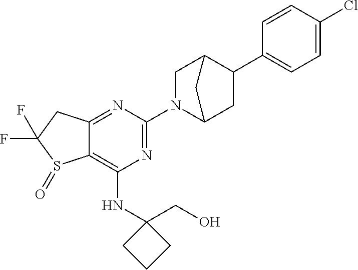 [SMILES: O=S1c2c(nc(N3CC4CC3CC4c3ccc(Cl)cc3)nc2NC2(CO)CCC2)CC1(F)F]

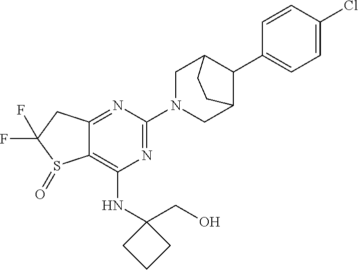 [SMILES: O=S1c2c(nc(N3CC4CCC(C3)C4c3ccc(Cl)cc3)nc2NC2(CO)CCC2)CC1(F)F]

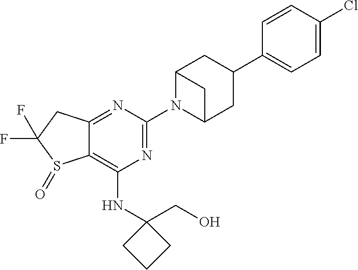 [SMILES: O=S1c2c(nc(N3C4CC(c5ccc(Cl)cc5)CC3C4)nc2NC2(CO)CCC2)CC1(F)F]

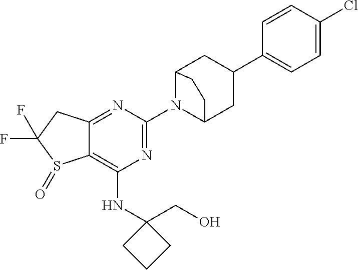 [SMILES: O=S1c2c(nc(N3C4CCC3CC(c3ccc(Cl)cc3)C4)nc2NC2(CO)CCC2)CC1(F)F]

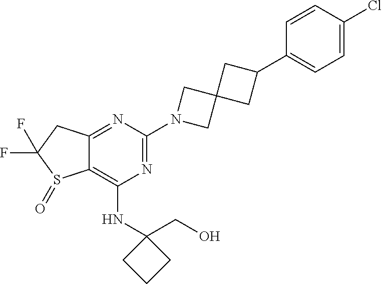 [SMILES: O=S1c2c(nc(N3CC4(CC(c5ccc(Cl)cc5)C4)C3)nc2NC2(CO)CCC2)CC1(F)F]

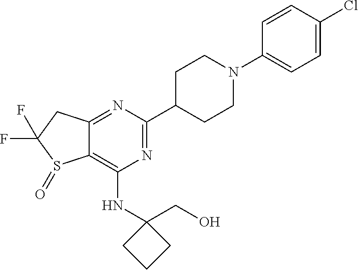 [SMILES: O=S1c2c(nc(C3CCN(c4ccc(Cl)cc4)CC3)nc2NC2(CO)CCC2)CC1(F)F]

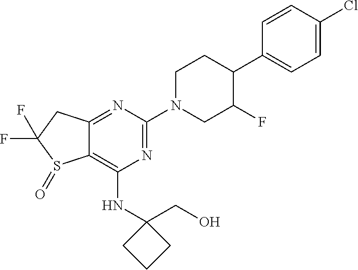 [SMILES: O=S1c2c(nc(N3CCC(c4ccc(Cl)cc4)C(F)C3)nc2NC2(CO)CCC2)CC1(F)F]

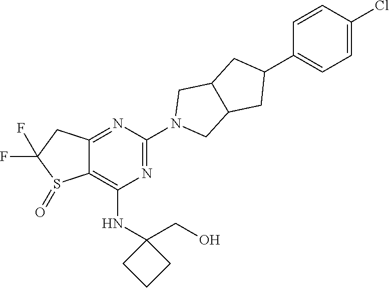 [SMILES: O=S1c2c(nc(N3CC4CC(c5ccc(Cl)cc5)CC4C3)nc2NC2(CO)CCC2)CC1(F)F]

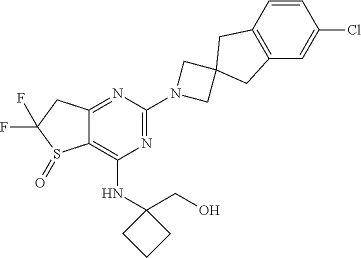 [SMILES: O=S1c2c(nc(N3CC4(Cc5ccc(Cl)cc5C4)C3)nc2NC2(CO)CCC2)CC1(F)F]

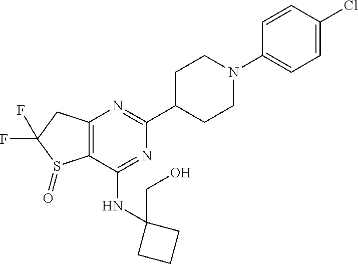 [SMILES: O=S1c2c(nc(C3CCN(c4ccc(Cl)cc4)CC3)nc2NC2(CO)CCC2)CC1(F)F]

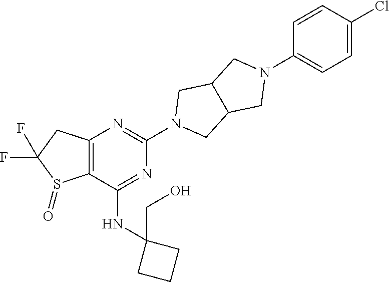 [SMILES: O=S1c2c(nc(N3CC4CN(c5ccc(Cl)cc5)CC4C3)nc2NC2(CO)CCC2)CC1(F)F]

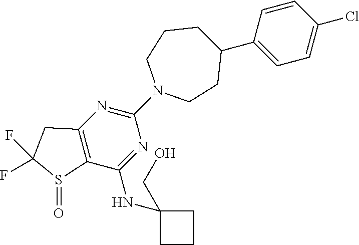 [SMILES: O=S1c2c(nc(N3CCCC(c4ccc(Cl)cc4)CC3)nc2NC2(CO)CCC2)CC1(F)F]

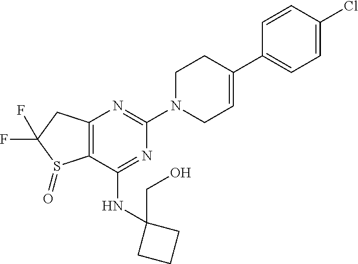 [SMILES: O=S1c2c(nc(N3CC=C(c4ccc(Cl)cc4)CC3)nc2NC2(CO)CCC2)CC1(F)F]

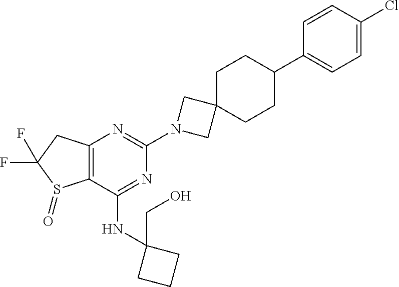 [SMILES: O=S1c2c(nc(N3CC4(CCC(c5ccc(Cl)cc5)CC4)C3)nc2NC2(CO)CCC2)CC1(F)F]

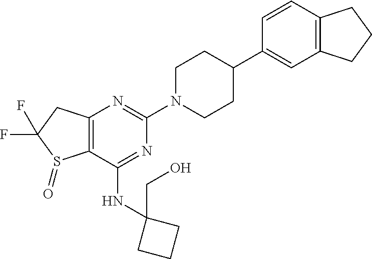 [SMILES: O=S1c2c(nc(N3CCC(c4ccc5c(c4)CCC5)CC3)nc2NC2(CO)CCC2)CC1(F)F]

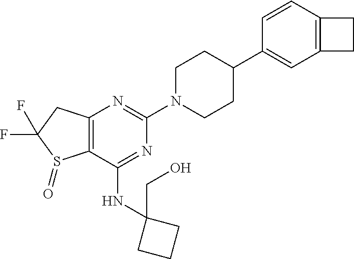 [SMILES: O=S1c2c(nc(N3CCC(c4ccc5c(c4)CC5)CC3)nc2NC2(CO)CCC2)CC1(F)F]

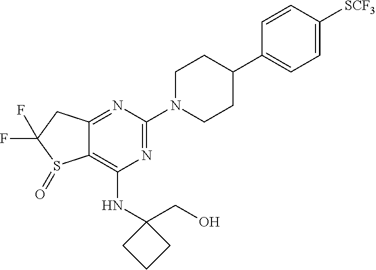

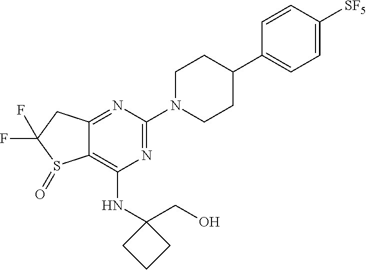

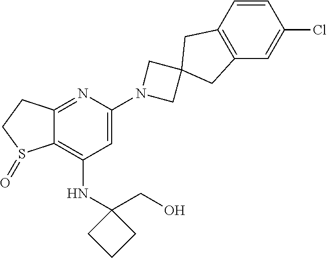 [SMILES: O=S1CCc2nc(N3CC4(Cc5ccc(Cl)cc5C4)C3)cc(NC3(CO)CCC3)c21]

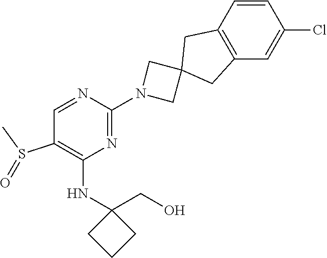 [SMILES: CS(=O)c1cnc(N2CC3(Cc4ccc(Cl)cc4C3)C2)nc1NC1(CO)CCC1]

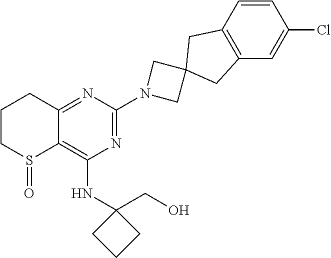 [SMILES: O=S1CCCc2nc(N3CC4(Cc5ccc(Cl)cc5C4)C3)nc(NC3(CO)CCC3)c21]

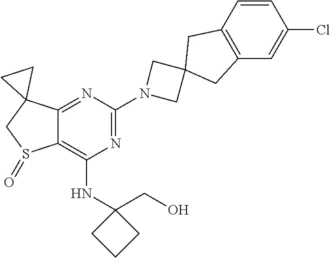 [SMILES: O=S1CC2(CC2)c2nc(N3CC4(Cc5ccc(Cl)cc5C4)C3)nc(NC3(CO)CCC3)c21]

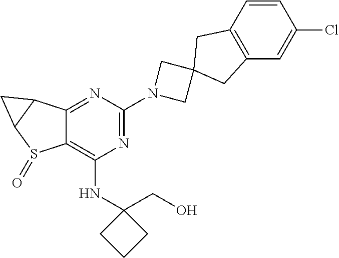 [SMILES: O=S1c2c(NC3(CO)CCC3)nc(N3CC4(Cc5ccc(Cl)cc5C4)C3)nc2C2CC21]

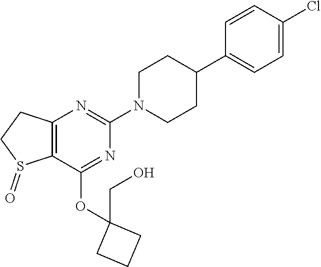 [SMILES: O=S1CCc2nc(N3CCC(c4ccc(Cl)cc4)CC3)nc(OC3(CO)CCC3)c21]

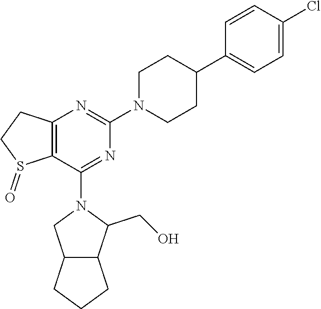 [SMILES: O=S1CCc2nc(N3CCC(c4ccc(Cl)cc4)CC3)nc(N3CC4CCCC4C3CO)c21]

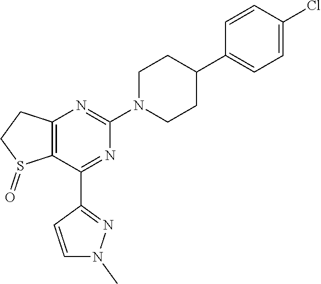 [SMILES: Cn1ccc(-c2nc(N3CCC(c4ccc(Cl)cc4)CC3)nc3c2S(=O)CC3)n1]

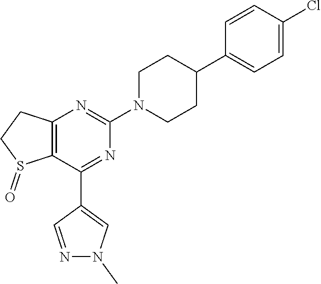 [SMILES: Cn1cc(-c2nc(N3CCC(c4ccc(Cl)cc4)CC3)nc3c2S(=O)CC3)cn1]

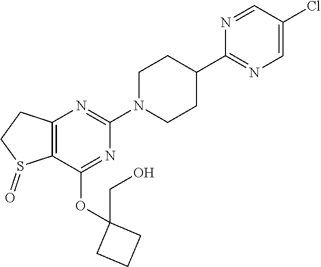 [SMILES: O=S1CCc2nc(N3CCC(c4ncc(Cl)cn4)CC3)nc(OC3(CO)CCC3)c21]

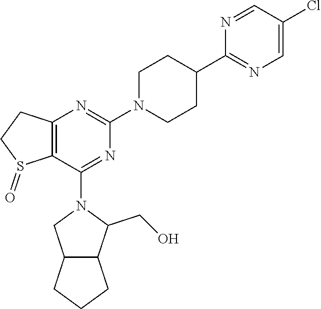 [SMILES: O=S1CCc2nc(N3CCC(c4ncc(Cl)cn4)CC3)nc(N3CC4CCCC4C3CO)c21]

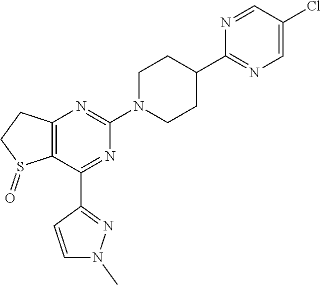 [SMILES: Cn1ccc(-c2nc(N3CCC(c4ncc(Cl)cn4)CC3)nc3c2S(=O)CC3)n1]

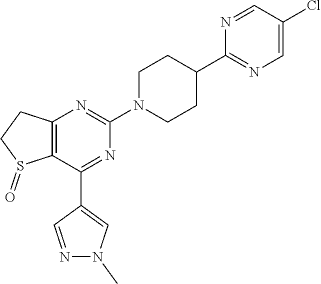 [SMILES: Cn1cc(-c2nc(N3CCC(c4ncc(Cl)cn4)CC3)nc3c2S(=O)CC3)cn1]

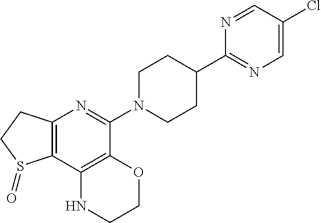 [SMILES: O=S1CCc2nc(N3CCC(c4ncc(Cl)cn4)CC3)c3c(c21)NCCO3]

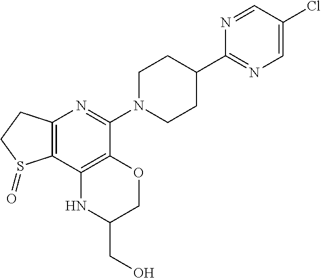 [SMILES: O=S1CCc2nc(N3CCC(c4ncc(Cl)cn4)CC3)c3c(c21)NC(CO)CO3]

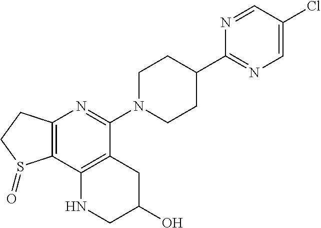 [SMILES: O=S1CCc2nc(N3CCC(c4ncc(Cl)cn4)CC3)c3c(c21)NCC(O)C3]

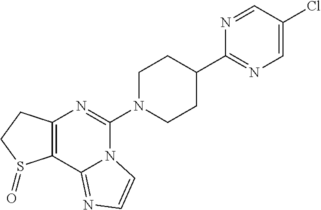 [SMILES: O=S1CCc2nc(N3CCC(c4ncc(Cl)cn4)CC3)n3ccnc3c21]

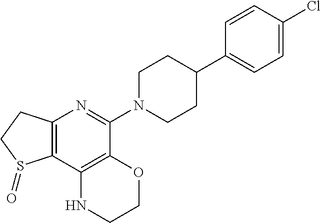 [SMILES: O=S1CCc2nc(N3CCC(c4ccc(Cl)cc4)CC3)c3c(c21)NCCO3]

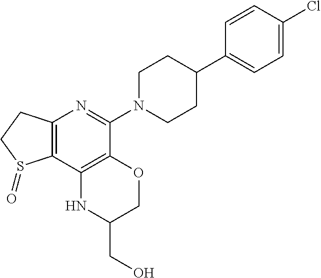 [SMILES: O=S1CCc2nc(N3CCC(c4ccc(Cl)cc4)CC3)c3c(c21)NC(CO)CO3]

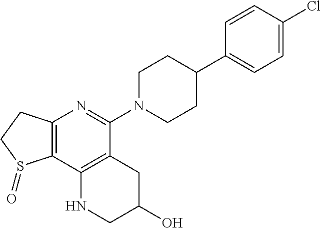 [SMILES: O=S1CCc2nc(N3CCC(c4ccc(Cl)cc4)CC3)c3c(c21)NCC(O)C3]

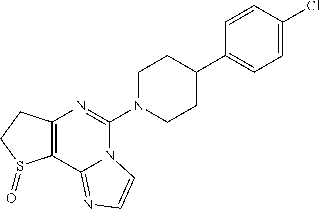 [SMILES: O=S1CCc2nc(N3CCC(c4ccc(Cl)cc4)CC3)n3ccnc3c21]

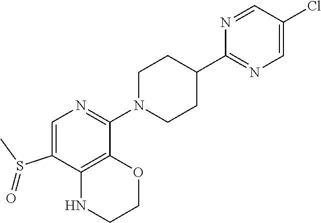 [SMILES: CS(=O)c1cnc(N2CCC(c3ncc(Cl)cn3)CC2)c2c1NCCO2]

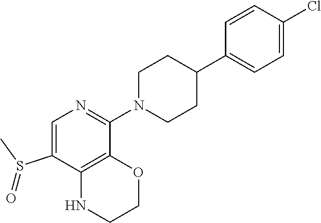 [SMILES: CS(=O)c1cnc(N2CCC(c3ccc(Cl)cc3)CC2)c2c1NCCO2]

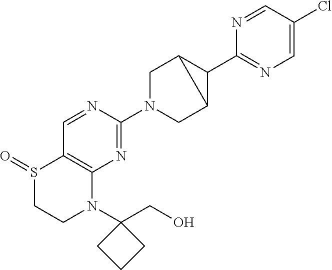 [SMILES: O=S1CCN(C2(CO)CCC2)c2nc(N3CC4C(C3)C4c3ncc(Cl)cn3)ncc21]

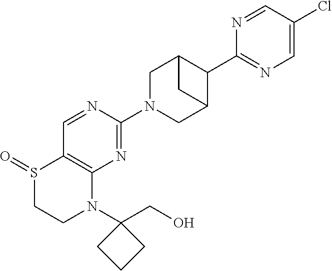 [SMILES: O=S1CCN(C2(CO)CCC2)c2nc(N3CC4CC(C3)C4c3ncc(Cl)cn3)ncc21]

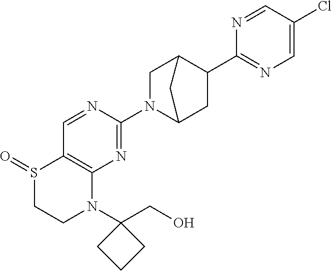 [SMILES: O=S1CCN(C2(CO)CCC2)c2nc(N3CC4CC3CC4c3ncc(Cl)cn3)ncc21]

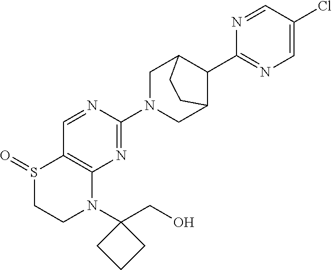 [SMILES: O=S1CCN(C2(CO)CCC2)c2nc(N3CC4CCC(C3)C4c3ncc(Cl)cn3)ncc21]

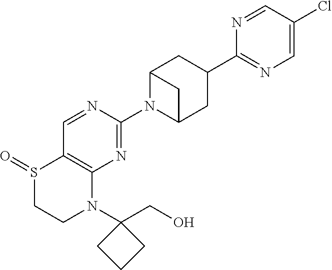 [SMILES: O=S1CCN(C2(CO)CCC2)c2nc(N3C4CC(c5ncc(Cl)cn5)CC3C4)ncc21]

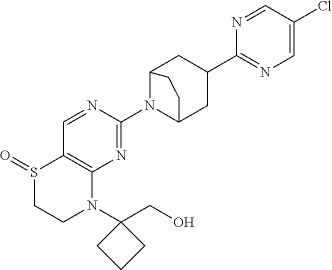 [SMILES: O=S1CCN(C2(CO)CCC2)c2nc(N3C4CCC3CC(c3ncc(Cl)cn3)C4)ncc21]

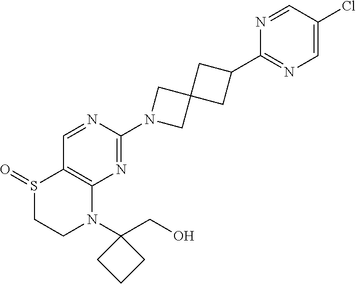 [SMILES: O=S1CCN(C2(CO)CCC2)c2nc(N3CC4(CC(c5ncc(Cl)cn5)C4)C3)ncc21]

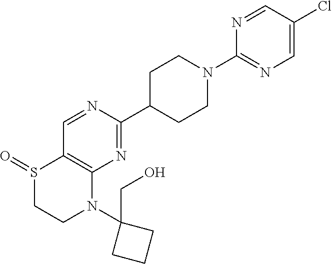 [SMILES: O=S1CCN(C2(CO)CCC2)c2nc(C3CCN(c4ncc(Cl)cn4)CC3)ncc21]

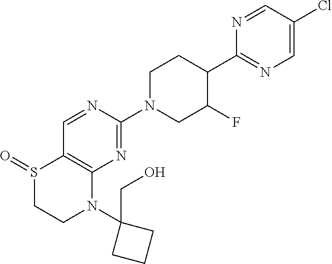 [SMILES: O=S1CCN(C2(CO)CCC2)c2nc(N3CCC(c4ncc(Cl)cn4)C(F)C3)ncc21]

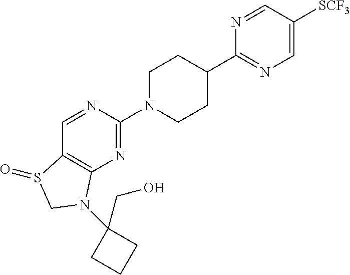

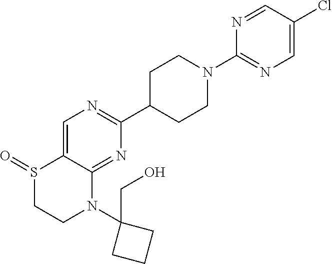 [SMILES: O=S1CCN(C2(CO)CCC2)c2nc(C3CCN(c4ncc(Cl)cn4)CC3)ncc21]

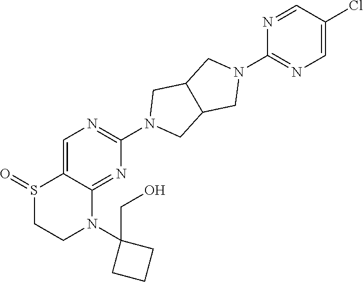 [SMILES: O=S1CCN(C2(CO)CCC2)c2nc(N3CC4CN(c5ncc(Cl)cn5)CC4C3)ncc21]

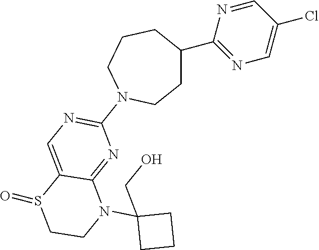 [SMILES: O=S1CCN(C2(CO)CCC2)c2nc(N3CCCC(c4ncc(Cl)cn4)CC3)ncc21]

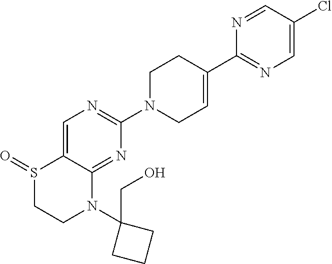 [SMILES: O=S1CCN(C2(CO)CCC2)c2nc(N3CC=C(c4ncc(Cl)cn4)CC3)ncc21]

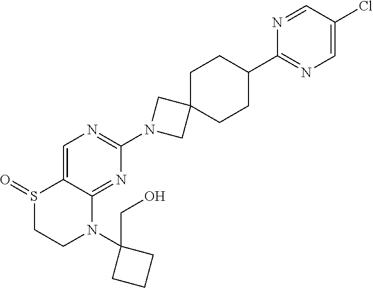 [SMILES: O=S1CCN(C2(CO)CCC2)c2nc(N3CC4(CCC(c5ncc(Cl)cn5)CC4)C3)ncc21]

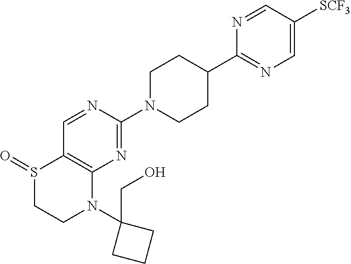

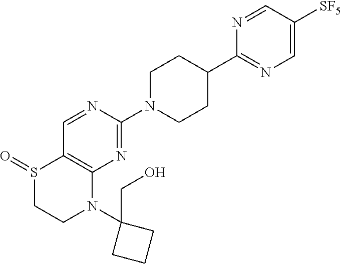

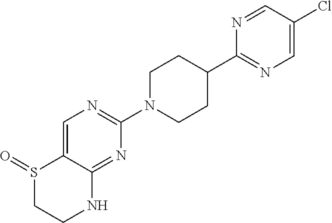 [SMILES: O=S1CCNc2nc(N3CCC(c4ncc(Cl)cn4)CC3)ncc21]

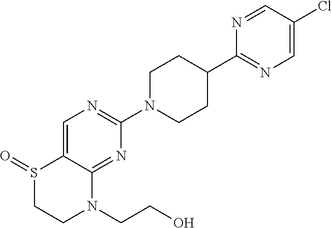 [SMILES: O=S1CCN(CCO)c2nc(N3CCC(c4ncc(Cl)cn4)CC3)ncc21]

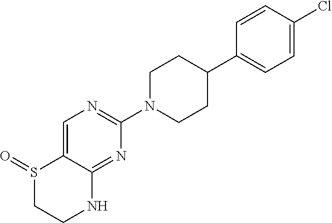 [SMILES: O=S1CCNc2nc(N3CCC(c4ccc(Cl)cc4)CC3)ncc21]

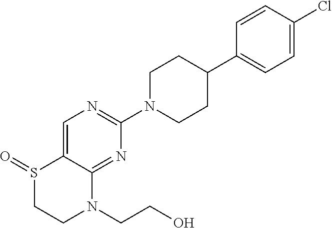 [SMILES: O=S1CCN(CCO)c2nc(N3CCC(c4ccc(Cl)cc4)CC3)ncc21]

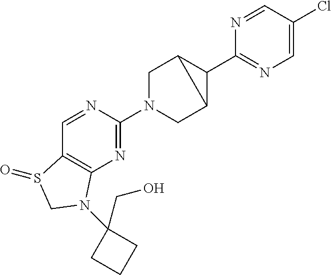 [SMILES: O=S1CN(C2(CO)CCC2)c2nc(N3CC4C(C3)C4c3ncc(Cl)cn3)ncc21]

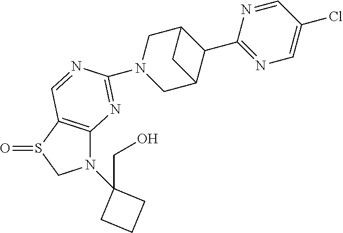 [SMILES: O=S1CN(C2(CO)CCC2)c2nc(N3CC4CC(C3)C4c3ncc(Cl)cn3)ncc21]

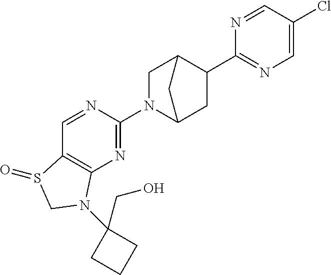 [SMILES: O=S1CN(C2(CO)CCC2)c2nc(N3CC4CC3CC4c3ncc(Cl)cn3)ncc21]

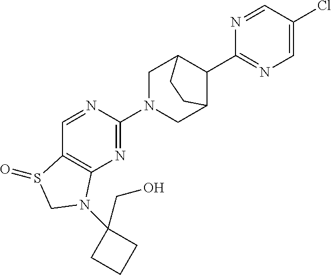 [SMILES: O=S1CN(C2(CO)CCC2)c2nc(N3CC4CCC(C3)C4c3ncc(Cl)cn3)ncc21]

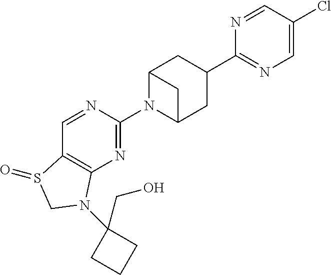 [SMILES: O=S1CN(C2(CO)CCC2)c2nc(N3C4CC(c5ncc(Cl)cn5)CC3C4)ncc21]

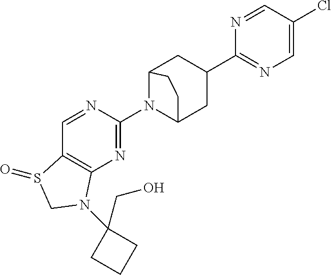 [SMILES: O=S1CN(C2(CO)CCC2)c2nc(N3C4CCC3CC(c3ncc(Cl)cn3)C4)ncc21]

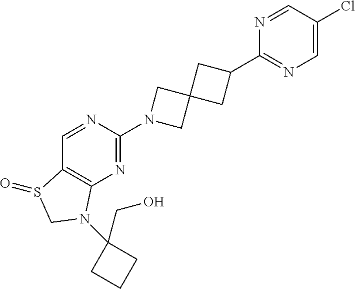 [SMILES: O=S1CN(C2(CO)CCC2)c2nc(N3CC4(CC(c5ncc(Cl)cn5)C4)C3)ncc21]

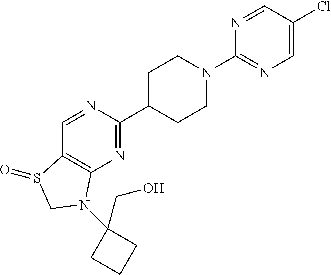 [SMILES: O=S1CN(C2(CO)CCC2)c2nc(C3CCN(c4ncc(Cl)cn4)CC3)ncc21]

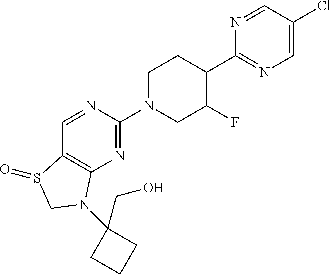 [SMILES: O=S1CN(C2(CO)CCC2)c2nc(N3CCC(c4ncc(Cl)cn4)C(F)C3)ncc21]

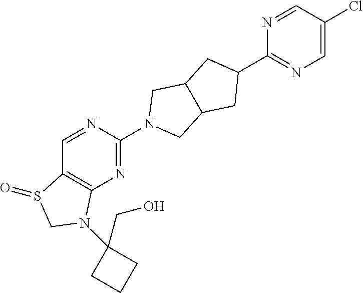 [SMILES: O=S1CN(C2(CO)CCC2)c2nc(N3CC4CC(c5ncc(Cl)cn5)CC4C3)ncc21]

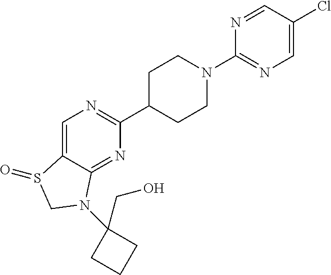 [SMILES: O=S1CN(C2(CO)CCC2)c2nc(C3CCN(c4ncc(Cl)cn4)CC3)ncc21]

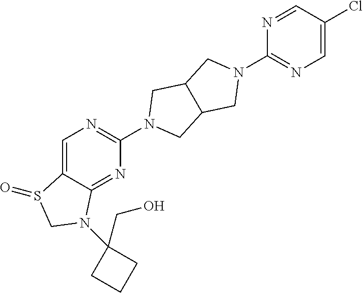 [SMILES: O=S1CN(C2(CO)CCC2)c2nc(N3CC4CN(c5ncc(Cl)cn5)CC4C3)ncc21]

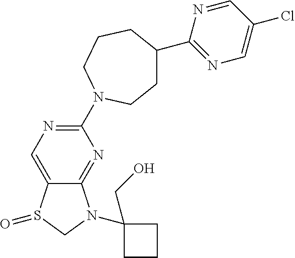 [SMILES: O=S1CN(C2(CO)CCC2)c2nc(N3CCCC(c4ncc(Cl)cn4)CC3)ncc21]

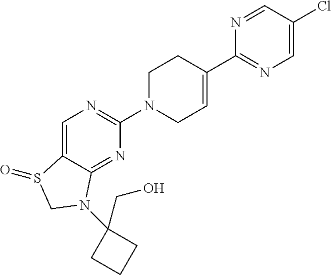 [SMILES: O=S1CN(C2(CO)CCC2)c2nc(N3CC=C(c4ncc(Cl)cn4)CC3)ncc21]

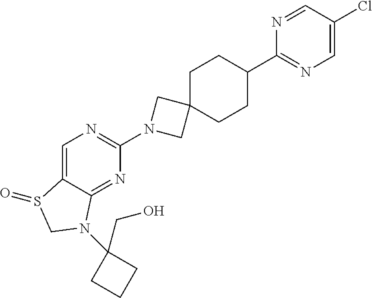 [SMILES: O=S1CN(C2(CO)CCC2)c2nc(N3CC4(CCC(c5ncc(Cl)cn5)CC4)C3)ncc21]

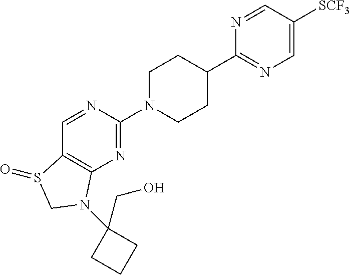

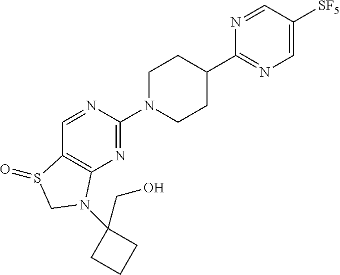

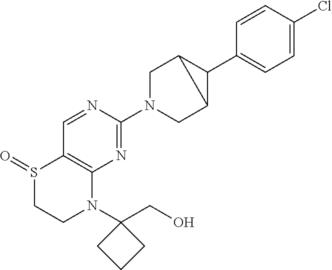 [SMILES: O=S1CCN(C2(CO)CCC2)c2nc(N3CC4C(C3)C4c3ccc(Cl)cc3)ncc21]

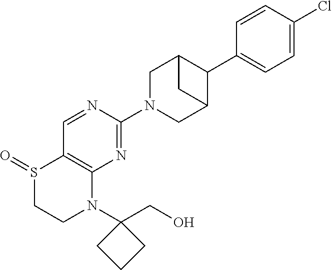 [SMILES: O=S1CCN(C2(CO)CCC2)c2nc(N3CC4CC(C3)C4c3ccc(Cl)cc3)ncc21]

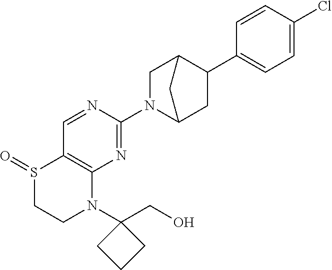 [SMILES: O=S1CCN(C2(CO)CCC2)c2nc(N3CC4CC3CC4c3ccc(Cl)cc3)ncc21]

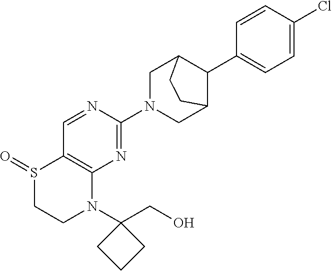 [SMILES: O=S1CCN(C2(CO)CCC2)c2nc(N3CC4CCC(C3)C4c3ccc(Cl)cc3)ncc21]

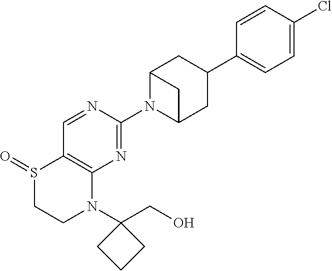 [SMILES: O=S1CCN(C2(CO)CCC2)c2nc(N3C4CC(c5ccc(Cl)cc5)CC3C4)ncc21]

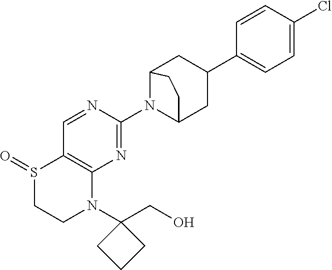 [SMILES: O=S1CCN(C2(CO)CCC2)c2nc(N3C4CCC3CC(c3ccc(Cl)cc3)C4)ncc21]

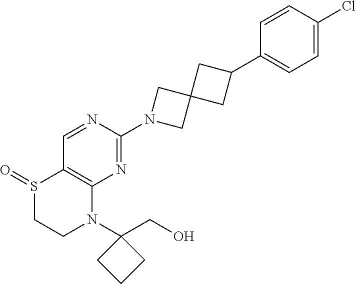 [SMILES: O=S1CCN(C2(CO)CCC2)c2nc(N3CC4(CC(c5ccc(Cl)cc5)C4)C3)ncc21]

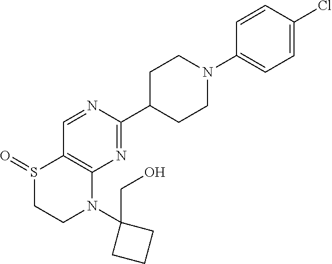 [SMILES: O=S1CCN(C2(CO)CCC2)c2nc(C3CCN(c4ccc(Cl)cc4)CC3)ncc21]

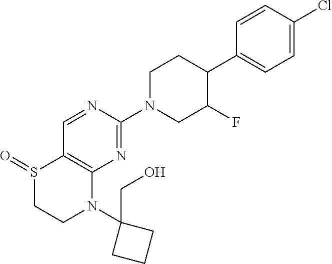 [SMILES: O=S1CCN(C2(CO)CCC2)c2nc(N3CCC(c4ccc(Cl)cc4)C(F)C3)ncc21]

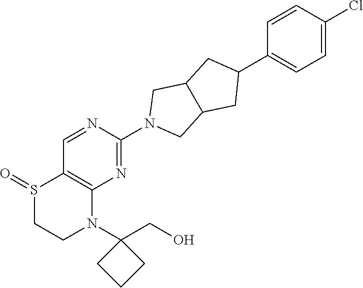 [SMILES: O=S1CCN(C2(CO)CCC2)c2nc(N3CC4CC(c5ccc(Cl)cc5)CC4C3)ncc21]

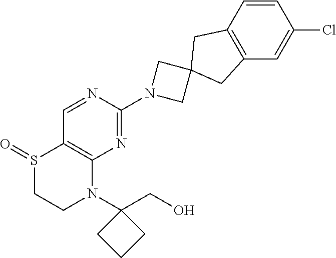 [SMILES: O=S1CCN(C2(CO)CCC2)c2nc(N3CC4(Cc5ccc(Cl)cc5C4)C3)ncc21]

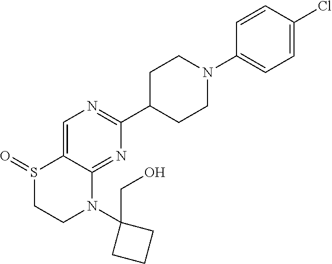 [SMILES: O=S1CCN(C2(CO)CCC2)c2nc(C3CCN(c4ccc(Cl)cc4)CC3)ncc21]

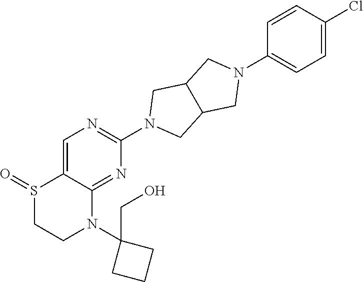 [SMILES: O=S1CCN(C2(CO)CCC2)c2nc(N3CC4CN(c5ccc(Cl)cc5)CC4C3)ncc21]

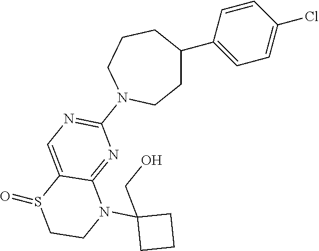 [SMILES: O=S1CCN(C2(CO)CCC2)c2nc(N3CCCC(c4ccc(Cl)cc4)CC3)ncc21]

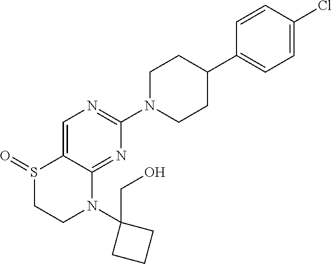 [SMILES: O=S1CCN(C2(CO)CCC2)c2nc(N3CCC(c4ccc(Cl)cc4)CC3)ncc21]

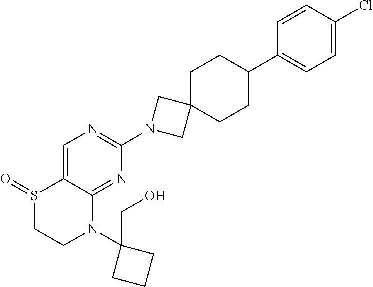 [SMILES: O=S1CCN(C2(CO)CCC2)c2nc(N3CC4(CCC(c5ccc(Cl)cc5)CC4)C3)ncc21]

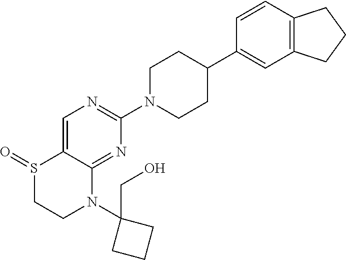 [SMILES: O=S1CCN(C2(CO)CCC2)c2nc(N3CCC(c4ccc5c(c4)CCC5)CC3)ncc21]

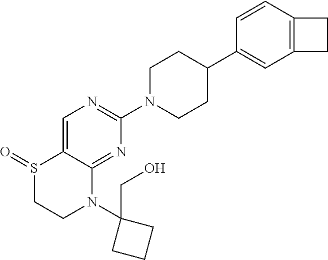 [SMILES: O=S1CCN(C2(CO)CCC2)c2nc(N3CCC(c4ccc5c(c4)CC5)CC3)ncc21]

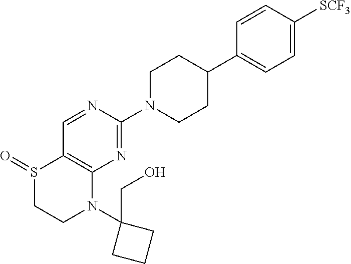

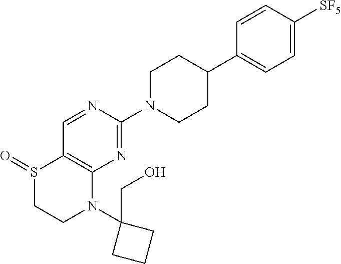

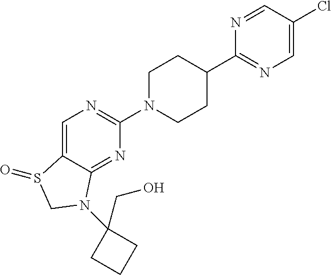 [SMILES: O=S1CN(C2(CO)CCC2)c2nc(N3CCC(c4ncc(Cl)cn4)CC3)ncc21]

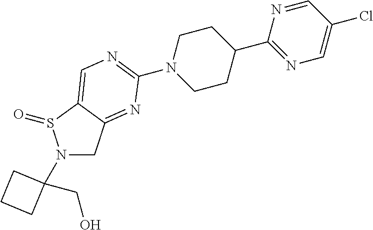 [SMILES: O=S1c2cnc(N3CCC(c4ncc(Cl)cn4)CC3)nc2CN1C1(CO)CCC1]

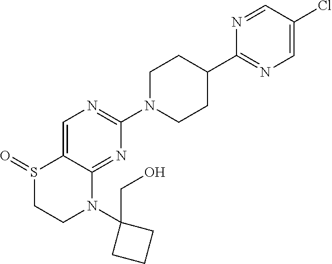 [SMILES: O=S1CCN(C2(CO)CCC2)c2nc(N3CCC(c4ncc(Cl)cn4)CC3)ncc21]

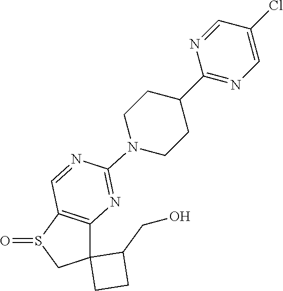 [SMILES: O=S1CC2(CCC2CO)c2nc(N3CCC(c4ncc(Cl)cn4)CC3)ncc21]

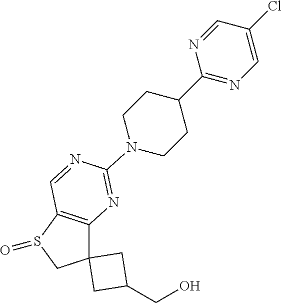 [SMILES: O=S1CC2(CC(CO)C2)c2nc(N3CCC(c4ncc(Cl)cn4)CC3)ncc21]

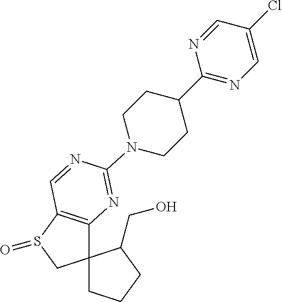 [SMILES: O=S1CC2(CCCC2CO)c2nc(N3CCC(c4ncc(Cl)cn4)CC3)ncc21]

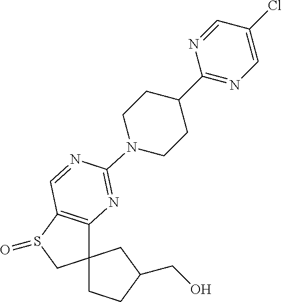 [SMILES: O=S1CC2(CCC(CO)C2)c2nc(N3CCC(c4ncc(Cl)cn4)CC3)ncc21]

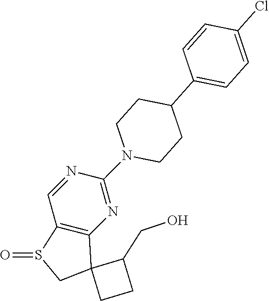 [SMILES: O=S1CC2(CCC2CO)c2nc(N3CCC(c4ccc(Cl)cc4)CC3)ncc21]

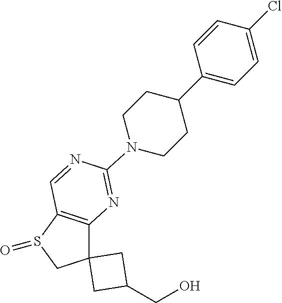 [SMILES: O=S1CC2(CC(CO)C2)c2nc(N3CCC(c4ccc(Cl)cc4)CC3)ncc21]

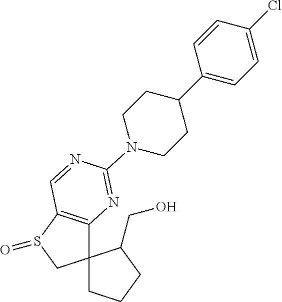 [SMILES: O=S1CC2(CCCC2CO)c2nc(N3CCC(c4ccc(Cl)cc4)CC3)ncc21]

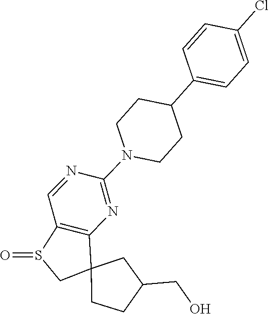 [SMILES: O=S1CC2(CCC(CO)C2)c2nc(N3CCC(c4ccc(Cl)cc4)CC3)ncc21]

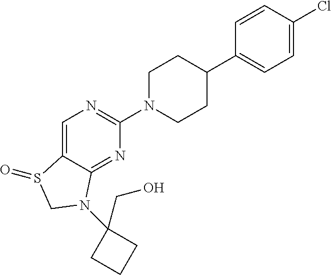 [SMILES: O=S1CN(C2(CO)CCC2)c2nc(N3CCC(c4ccc(Cl)cc4)CC3)ncc21]

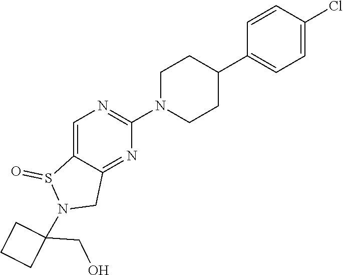 [SMILES: O=S1c2cnc(N3CCC(c4ccc(Cl)cc4)CC3)nc2CN1C1(CO)CCC1]

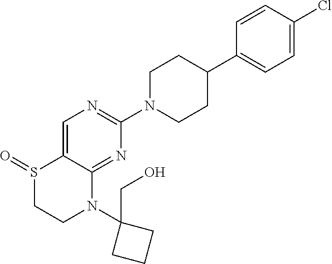 [SMILES: O=S1CCN(C2(CO)CCC2)c2nc(N3CCC(c4ccc(Cl)cc4)CC3)ncc21]

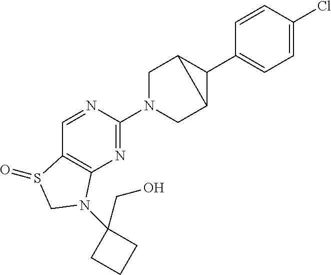 [SMILES: O=S1CN(C2(CO)CCC2)c2nc(N3CC4C(C3)C4c3ccc(Cl)cc3)ncc21]

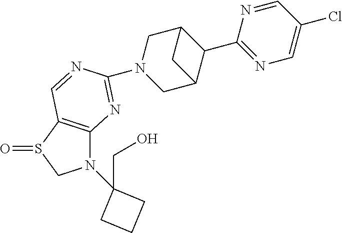 [SMILES: O=S1CN(C2(CO)CCC2)c2nc(N3CC4CC(C3)C4c3ncc(Cl)cn3)ncc21]

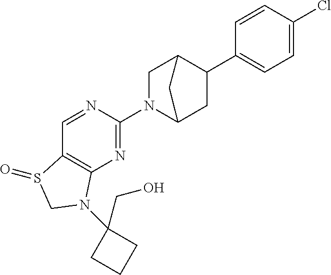 [SMILES: O=S1CN(C2(CO)CCC2)c2nc(N3CC4CC3CC4c3ccc(Cl)cc3)ncc21]

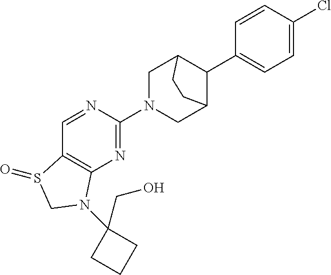 [SMILES: O=S1CN(C2(CO)CCC2)c2nc(N3CC4CCC(C3)C4c3ccc(Cl)cc3)ncc21]

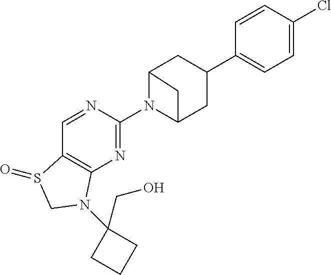 [SMILES: O=S1CN(C2(CO)CCC2)c2nc(N3C4CC(c5ccc(Cl)cc5)CC3C4)ncc21]

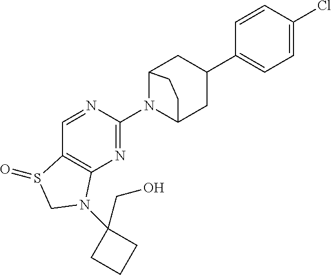 [SMILES: O=S1CN(C2(CO)CCC2)c2nc(N3C4CCC3CC(c3ccc(Cl)cc3)C4)ncc21]

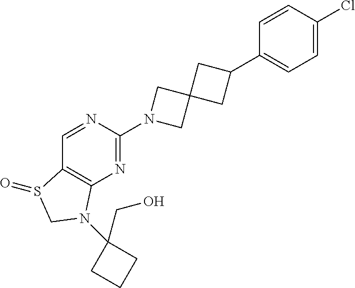 [SMILES: O=S1CN(C2(CO)CCC2)c2nc(N3CC4(CC(c5ccc(Cl)cc5)C4)C3)ncc21]

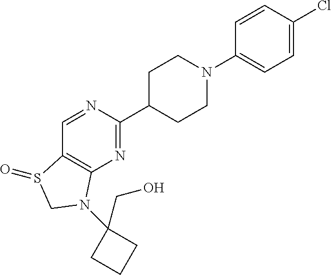 [SMILES: O=S1CN(C2(CO)CCC2)c2nc(C3CCN(c4ccc(Cl)cc4)CC3)ncc21]

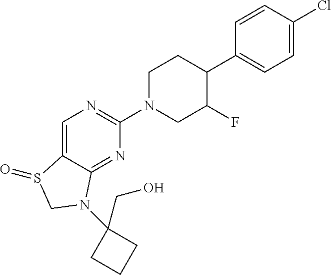 [SMILES: O=S1CN(C2(CO)CCC2)c2nc(N3CCC(c4ccc(Cl)cc4)C(F)C3)ncc21]

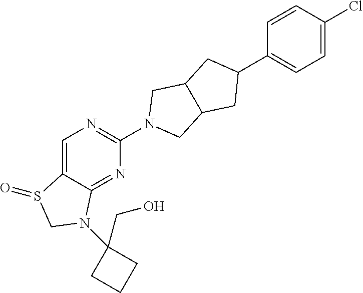 [SMILES: O=S1CN(C2(CO)CCC2)c2nc(N3CC4CC(c5ccc(Cl)cc5)CC4C3)ncc21]

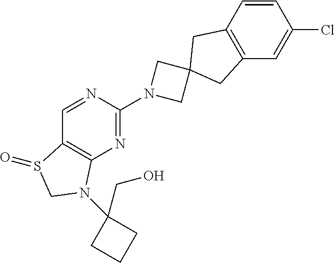 [SMILES: O=S1CN(C2(CO)CCC2)c2nc(N3CC4(Cc5ccc(Cl)cc5C4)C3)ncc21]

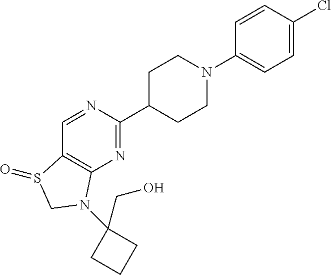 [SMILES: O=S1CN(C2(CO)CCC2)c2nc(C3CCN(c4ccc(Cl)cc4)CC3)ncc21]

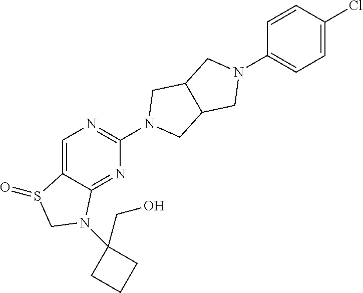 [SMILES: O=S1CN(C2(CO)CCC2)c2nc(N3CC4CN(c5ccc(Cl)cc5)CC4C3)ncc21]

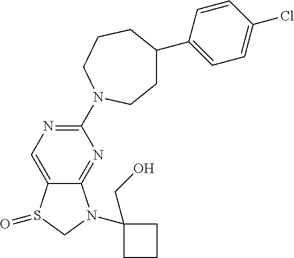 [SMILES: O=S1CN(C2(CO)CCC2)c2nc(N3CCCC(c4ccc(Cl)cc4)CC3)ncc21]

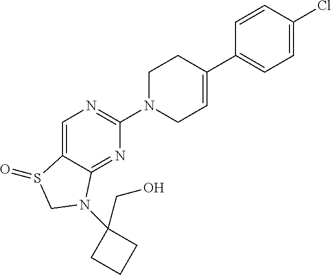 [SMILES: O=S1CN(C2(CO)CCC2)c2nc(N3CC=C(c4ccc(Cl)cc4)CC3)ncc21]

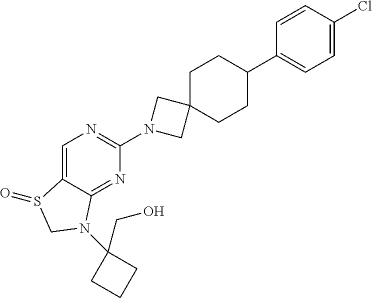 [SMILES: O=S1CN(C2(CO)CCC2)c2nc(N3CC4(CCC(c5ccc(Cl)cc5)CC4)C3)ncc21]

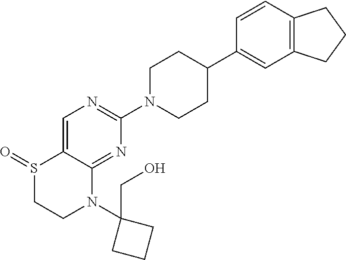 [SMILES: O=S1CCN(C2(CO)CCC2)c2nc(N3CCC(c4ccc5c(c4)CCC5)CC3)ncc21]

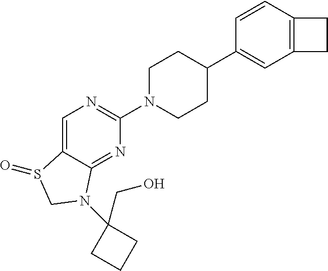 [SMILES: O=S1CN(C2(CO)CCC2)c2nc(N3CCC(c4ccc5c(c4)CC5)CC3)ncc21]

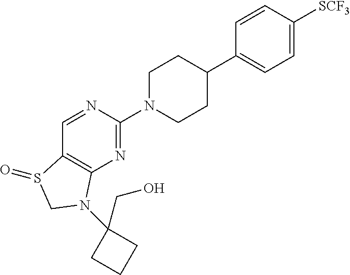

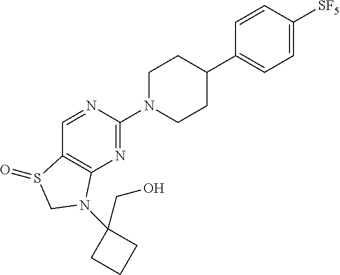

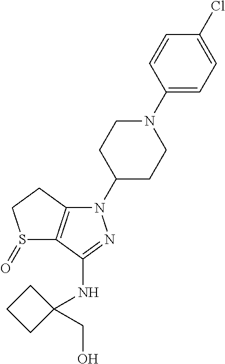 [SMILES: O=S1CCc2c1c(NC1(CO)CCC1)nn2C1CCN(c2ccc(Cl)cc2)CC1]

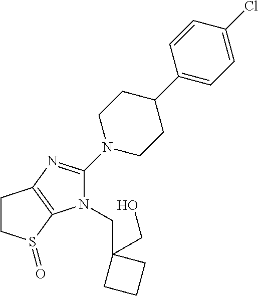 [SMILES: O=S1CCc2nc(N3CCC(c4ccc(Cl)cc4)CC3)n(CC3(CO)CCC3)c21]

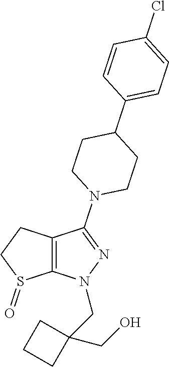 [SMILES: O=S1CCc2c(N3CCC(c4ccc(Cl)cc4)CC3)nn(CC3(CO)CCC3)c21]

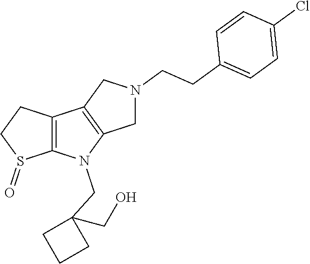 [SMILES: O=S1CCc2c3c(n(CC4(CO)CCC4)c21)CN(CCc1ccc(Cl)cc1)C3]

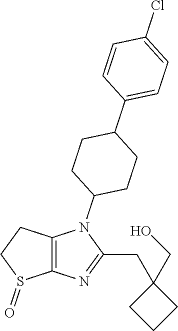 [SMILES: O=S1CCc2c1nc(CC1(CO)CCC1)n2C1CCC(c2ccc(Cl)cc2)CC1]

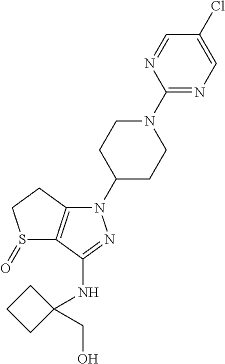 [SMILES: O=S1CCc2c1c(NC1(CO)CCC1)nn2C1CCN(c2ncc(Cl)cn2)CC1]

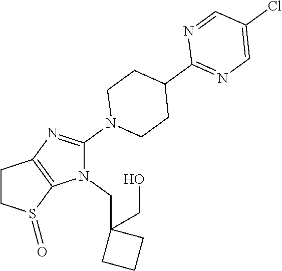 [SMILES: O=S1CCc2nc(N3CCC(c4ncc(Cl)cn4)CC3)n(CC3(CO)CCC3)c21]

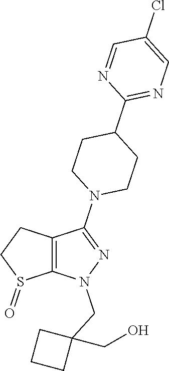 [SMILES: O=S1CCc2c(N3CCC(c4ncc(Cl)cn4)CC3)nn(CC3(CO)CCC3)c21]

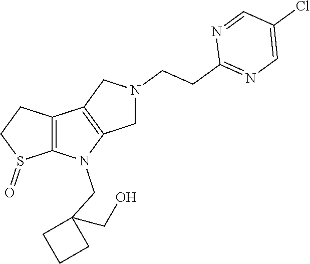 [SMILES: O=S1CCc2c3c(n(CC4(CO)CCC4)c21)CN(CCc1ncc(Cl)cn1)C3]

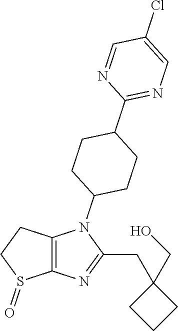 [SMILES: O=S1CCc2c1nc(CC1(CO)CCC1)n2C1CCC(c2ncc(Cl)cn2)CC1]

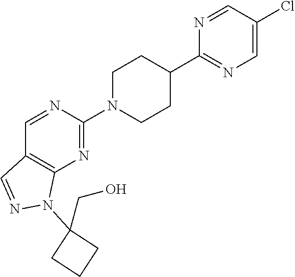 [SMILES: OCC1(n2ncc3cnc(N4CCC(c5ncc(Cl)cn5)CC4)nc32)CCC1]

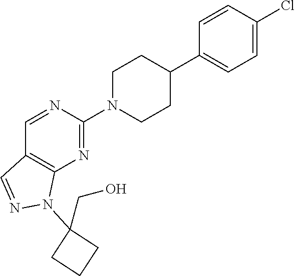 [SMILES: OCC1(n2ncc3cnc(N4CCC(c5ccc(Cl)cc5)CC4)nc32)CCC1]

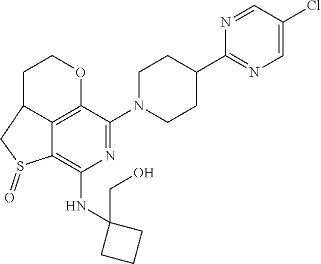 [SMILES: O=S1CC2CCOc3c(N4CCC(c5ncc(Cl)cn5)CC4)nc(NC4(CO)CCC4)c1c32]

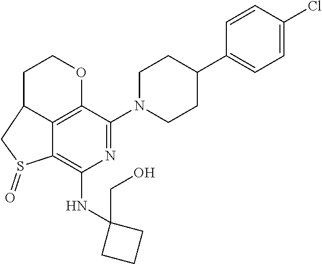 [SMILES: O=S1CC2CCOc3c(N4CCC(c5ccc(Cl)cc5)CC4)nc(NC4(CO)CCC4)c1c32]

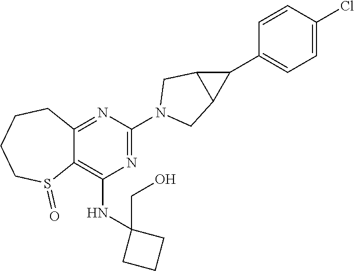 [SMILES: O=S1CCCCc2nc(N3CC4C(C3)C4c3ccc(Cl)cc3)nc(NC3(CO)CCC3)c21]

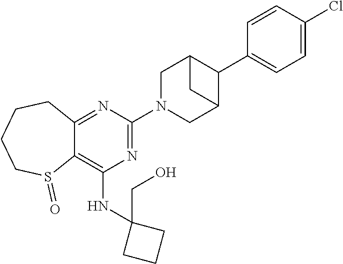 [SMILES: O=S1CCCCc2nc(N3CC4CC(C3)C4c3ccc(Cl)cc3)nc(NC3(CO)CCC3)c21]

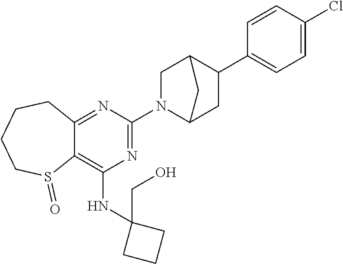 [SMILES: O=S1CCCCc2nc(N3CC4CC3CC4c3ccc(Cl)cc3)nc(NC3(CO)CCC3)c21]

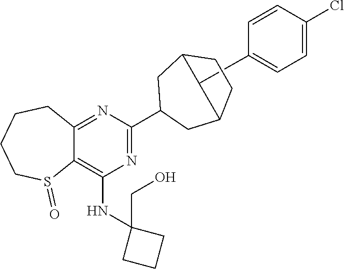 [SMILES: O=S1CCCCc2nc(C3CC4CCC(C3)C4c3ccc(Cl)cc3)nc(NC3(CO)CCC3)c21]

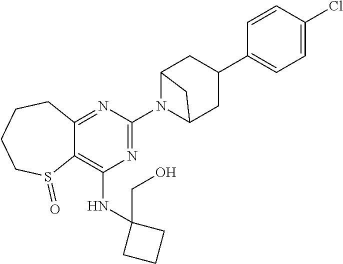 [SMILES: O=S1CCCCc2nc(N3C4CC(c5ccc(Cl)cc5)CC3C4)nc(NC3(CO)CCC3)c21]

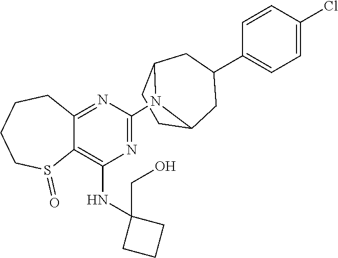 [SMILES: O=S1CCCCc2nc(N3C4CCC3CC(c3ccc(Cl)cc3)C4)nc(NC3(CO)CCC3)c21]

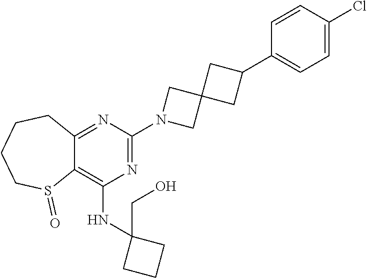 [SMILES: O=S1CCCCc2nc(N3CC4(CC(c5ccc(Cl)cc5)C4)C3)nc(NC3(CO)CCC3)c21]

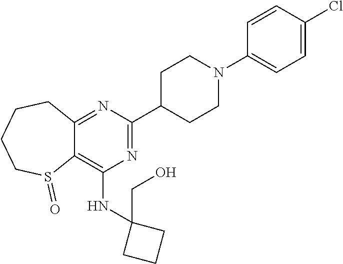 [SMILES: O=S1CCCCc2nc(C3CCN(c4ccc(Cl)cc4)CC3)nc(NC3(CO)CCC3)c21]

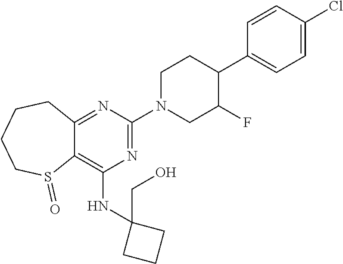 [SMILES: O=S1CCCCc2nc(N3CCC(c4ccc(Cl)cc4)C(F)C3)nc(NC3(CO)CCC3)c21]

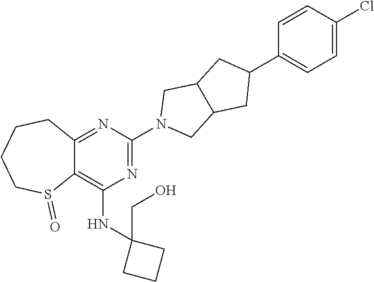 [SMILES: O=S1CCCCc2nc(N3CC4CC(c5ccc(Cl)cc5)CC4C3)nc(NC3(CO)CCC3)c21]

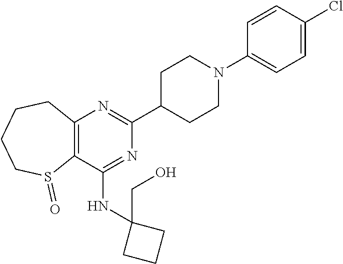 [SMILES: O=S1CCCCc2nc(C3CCN(c4ccc(Cl)cc4)CC3)nc(NC3(CO)CCC3)c21]

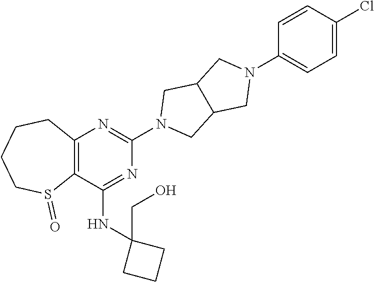 [SMILES: O=S1CCCCc2nc(N3CC4CN(c5ccc(Cl)cc5)CC4C3)nc(NC3(CO)CCC3)c21]

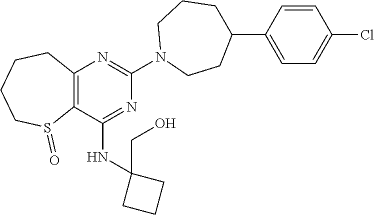 [SMILES: O=S1CCCCc2nc(N3CCCC(c4ccc(Cl)cc4)CC3)nc(NC3(CO)CCC3)c21]

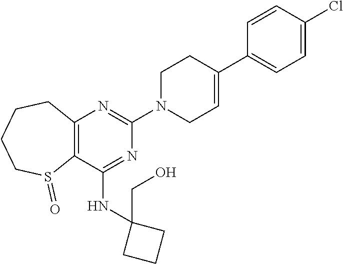 [SMILES: O=S1CCCCc2nc(N3CC=C(c4ccc(Cl)cc4)CC3)nc(NC3(CO)CCC3)c21]

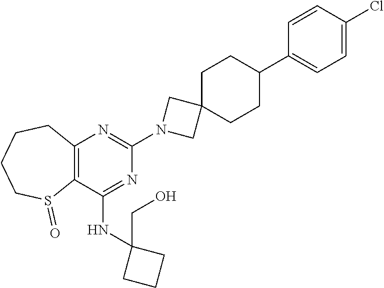 [SMILES: O=S1CCCCc2nc(N3CC4(CCC(c5ccc(Cl)cc5)CC4)C3)nc(NC3(CO)CCC3)c21]

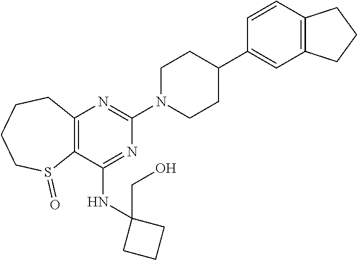 [SMILES: O=S1CCCCc2nc(N3CCC(c4ccc5c(c4)CCC5)CC3)nc(NC3(CO)CCC3)c21]

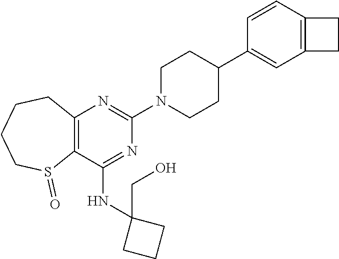 [SMILES: O=S1CCCCc2nc(N3CCC(c4ccc5c(c4)CC5)CC3)nc(NC3(CO)CCC3)c21]

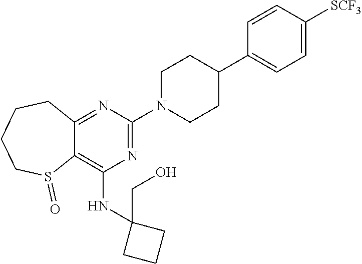

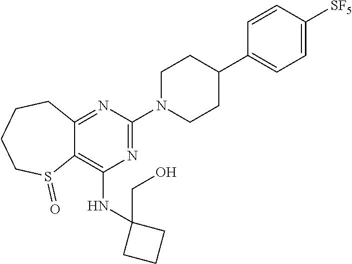

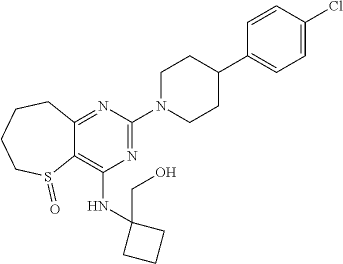 [SMILES: O=S1CCCCc2nc(N3CCC(c4ccc(Cl)cc4)CC3)nc(NC3(CO)CCC3)c21]

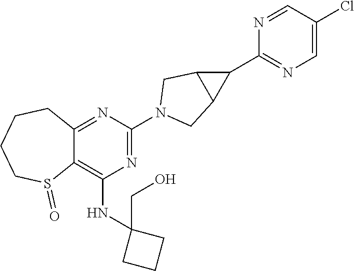 [SMILES: O=S1CCCCc2nc(N3CC4C(C3)C4c3ncc(Cl)cn3)nc(NC3(CO)CCC3)c21]

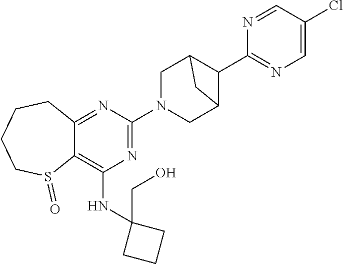 [SMILES: O=S1CCCCc2nc(N3CC4CC(C3)C4c3ncc(Cl)cn3)nc(NC3(CO)CCC3)c21]

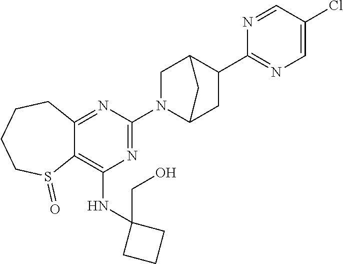 [SMILES: O=S1CCCCc2nc(N3CC4CC3CC4c3ncc(Cl)cn3)nc(NC3(CO)CCC3)c21]

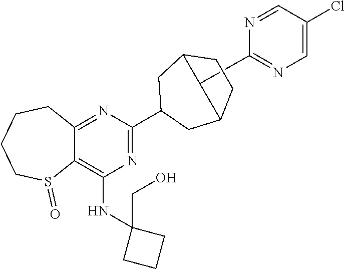 [SMILES: O=S1CCCCc2nc(C3CC4CCC(C3)C4c3ncc(Cl)cn3)nc(NC3(CO)CCC3)c21]

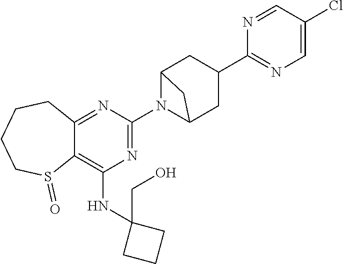 [SMILES: O=S1CCCCc2nc(N3C4CC(c5ncc(Cl)cn5)CC3C4)nc(NC3(CO)CCC3)c21]

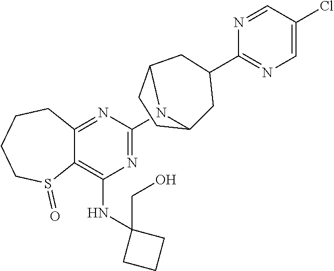 [SMILES: O=S1CCCCc2nc(N3C4CCC3CC(c3ncc(Cl)cn3)C4)nc(NC3(CO)CCC3)c21]

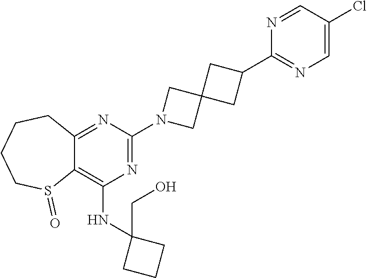 [SMILES: O=S1CCCCc2nc(N3CC4(CC(c5ncc(Cl)cn5)C4)C3)nc(NC3(CO)CCC3)c21]

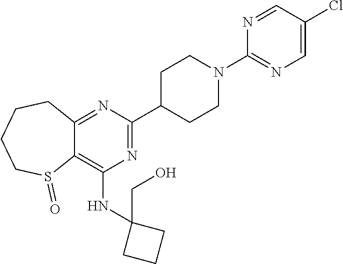 [SMILES: O=S1CCCCc2nc(C3CCN(c4ncc(Cl)cn4)CC3)nc(NC3(CO)CCC3)c21]

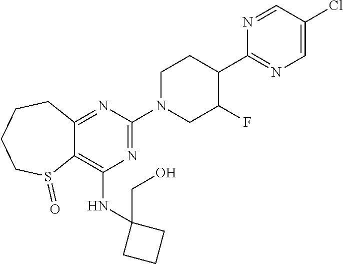 [SMILES: O=S1CCCCc2nc(N3CCC(c4ncc(Cl)cn4)C(F)C3)nc(NC3(CO)CCC3)c21]

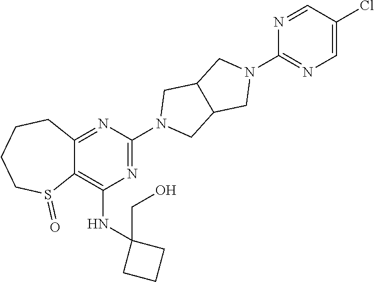 [SMILES: O=S1CCCCc2nc(N3CC4CN(c5ncc(Cl)cn5)CC4C3)nc(NC3(CO)CCC3)c21]

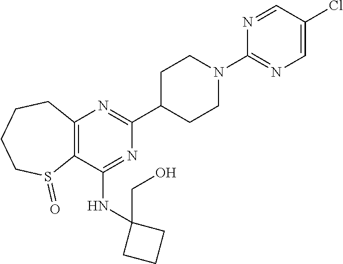 [SMILES: O=S1CCCCc2nc(C3CCN(c4ncc(Cl)cn4)CC3)nc(NC3(CO)CCC3)c21]

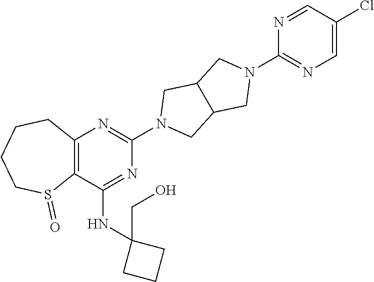 [SMILES: O=S1CCCCc2nc(N3CC4CN(c5ncc(Cl)cn5)CC4C3)nc(NC3(CO)CCC3)c21]

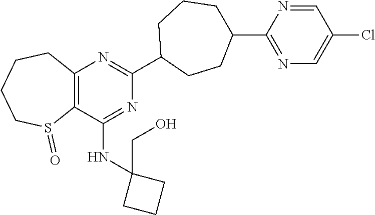 [SMILES: O=S1CCCCc2nc(C3CCCC(c4ncc(Cl)cn4)CC3)nc(NC3(CO)CCC3)c21]

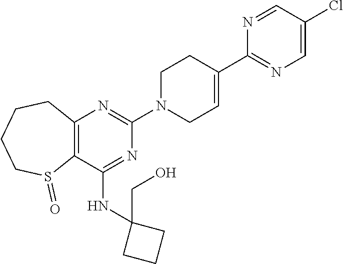 [SMILES: O=S1CCCCc2nc(N3CC=C(c4ncc(Cl)cn4)CC3)nc(NC3(CO)CCC3)c21]

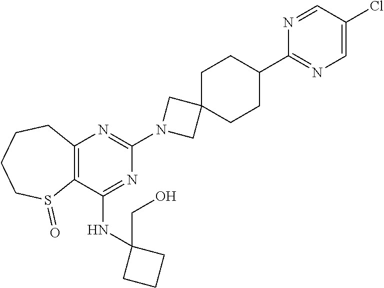 [SMILES: O=S1CCCCc2nc(N3CC4(CCC(c5ncc(Cl)cn5)CC4)C3)nc(NC3(CO)CCC3)c21]

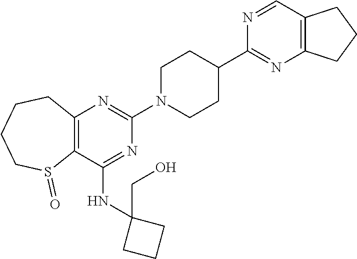 [SMILES: O=S1CCCCc2nc(N3CCC(c4ncc5c(n4)CCC5)CC3)nc(NC3(CO)CCC3)c21]

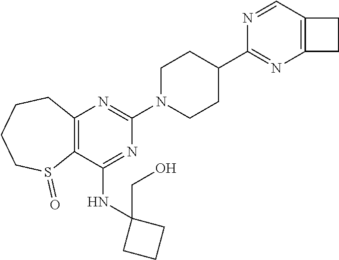 [SMILES: O=S1CCCCc2nc(N3CCC(c4ncc5c(n4)CC5)CC3)nc(NC3(CO)CCC3)c21]

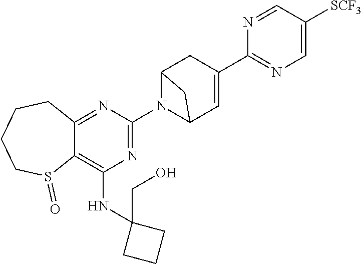

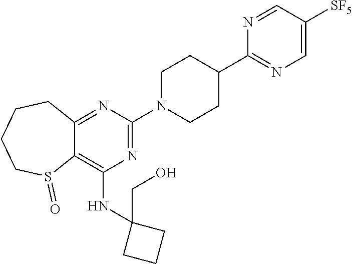

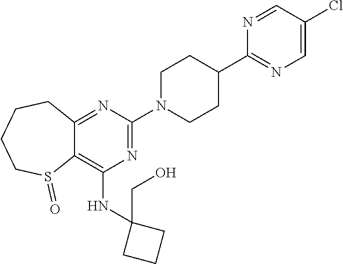 [SMILES: O=S1CCCCc2nc(N3CCC(c4ncc(Cl)cn4)CC3)nc(NC3(CO)CCC3)c21]

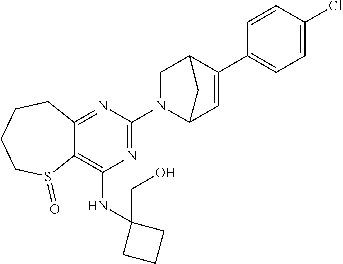 [SMILES: O=S1CCCCc2nc(N3CC4CC3C=C4c3ccc(Cl)cc3)nc(NC3(CO)CCC3)c21]

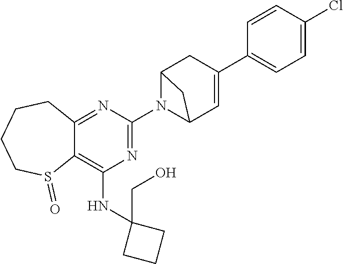 [SMILES: O=S1CCCCc2nc(N3C4C=C(c5ccc(Cl)cc5)CC3C4)nc(NC3(CO)CCC3)c21]

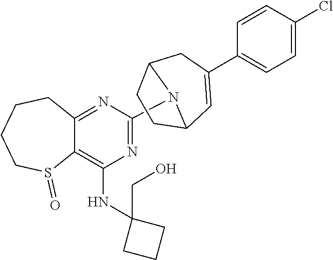 [SMILES: O=S1CCCCc2nc(N3C4C=C(c5ccc(Cl)cc5)CC3CC4)nc(NC3(CO)CCC3)c21]

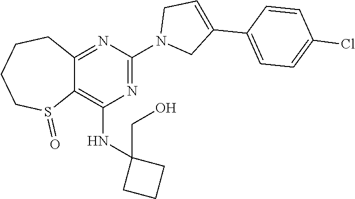 [SMILES: O=S1CCCCc2nc(N3CC=C(c4ccc(Cl)cc4)C3)nc(NC3(CO)CCC3)c21]

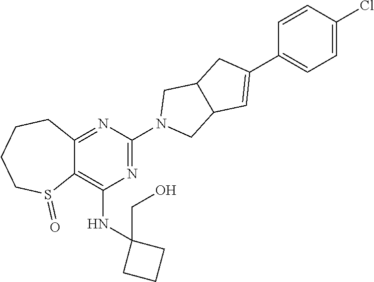 [SMILES: O=S1CCCCc2nc(N3CC4C=C(c5ccc(Cl)cc5)CC4C3)nc(NC3(CO)CCC3)c21]

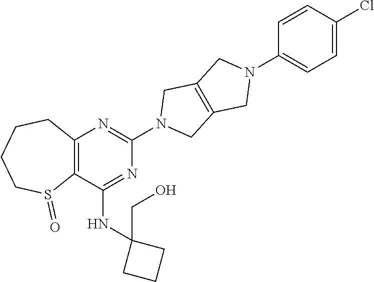 [SMILES: O=S1CCCCc2nc(N3CC4=C(CN(c5ccc(Cl)cc5)C4)C3)nc(NC3(CO)CCC3)c21]

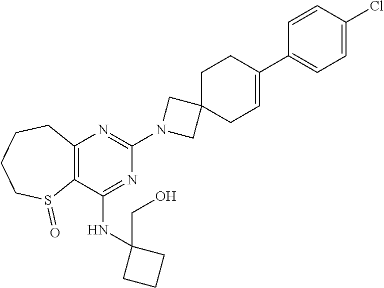 [SMILES: O=S1CCCCc2nc(N3CC4(CC=C(c5ccc(Cl)cc5)CC4)C3)nc(NC3(CO)CCC3)c21]

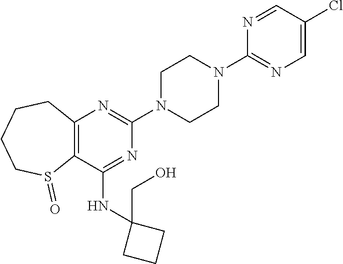 [SMILES: O=S1CCCCc2nc(N3CCN(c4ncc(Cl)cn4)CC3)nc(NC3(CO)CCC3)c21]

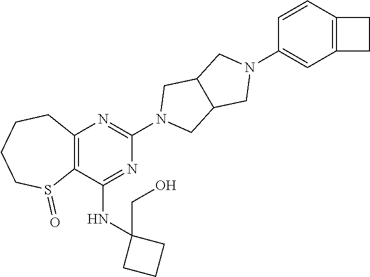 [SMILES: O=S1CCCCc2nc(N3CC4CN(c5ccc6c(c5)CC6)CC4C3)nc(NC3(CO)CCC3)c21]

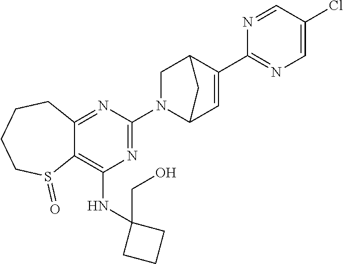 [SMILES: O=S1CCCCc2nc(N3CC4CC3C=C4c3ncc(Cl)cn3)nc(NC3(CO)CCC3)c21]

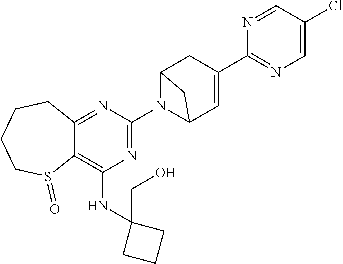 [SMILES: O=S1CCCCc2nc(N3C4C=C(c5ncc(Cl)cn5)CC3C4)nc(NC3(CO)CCC3)c21]

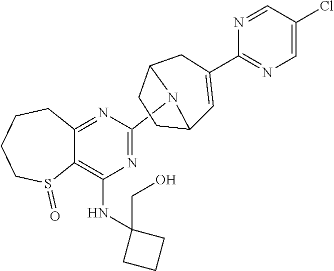 [SMILES: O=S1CCCCc2nc(N3C4C=C(c5ncc(Cl)cn5)CC3CC4)nc(NC3(CO)CCC3)c21]

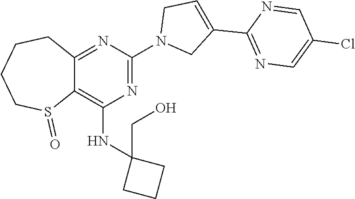 [SMILES: O=S1CCCCc2nc(N3CC=C(c4ncc(Cl)cn4)C3)nc(NC3(CO)CCC3)c21]

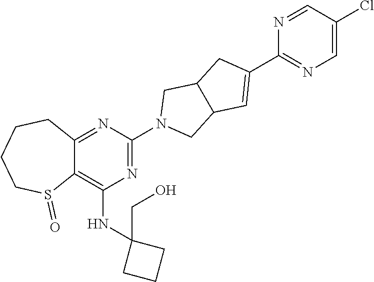 [SMILES: O=S1CCCCc2nc(N3CC4C=C(c5ncc(Cl)cn5)CC4C3)nc(NC3(CO)CCC3)c21]

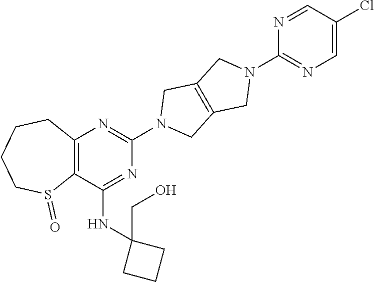 [SMILES: O=S1CCCCc2nc(N3CC4=C(CN(c5ncc(Cl)cn5)C4)C3)nc(NC3(CO)CCC3)c21]

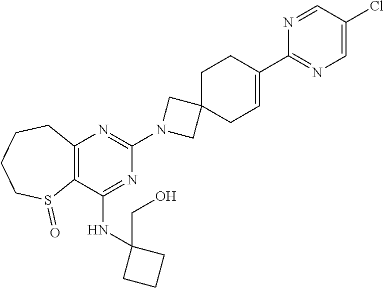 [SMILES: O=S1CCCCc2nc(N3CC4(CC=C(c5ncc(Cl)cn5)CC4)C3)nc(NC3(CO)CCC3)c21]

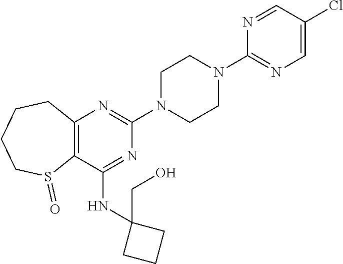 [SMILES: O=S1CCCCc2nc(N3CCN(c4ncc(Cl)cn4)CC3)nc(NC3(CO)CCC3)c21]

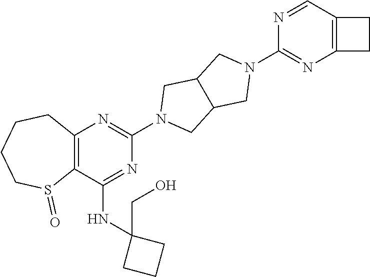 [SMILES: O=S1CCCCc2nc(N3CC4CN(c5ncc6c(n5)CC6)CC4C3)nc(NC3(CO)CCC3)c21]

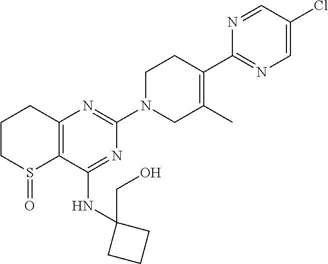 [SMILES: CC1=C(c2ncc(Cl)cn2)CCN(c2nc3c(c(NC4(CO)CCC4)n2)S(=O)CCC3)C1]

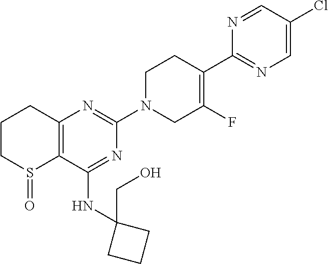 [SMILES: O=S1CCCc2nc(N3CCC(c4ncc(Cl)cn4)=C(F)C3)nc(NC3(CO)CCC3)c21]

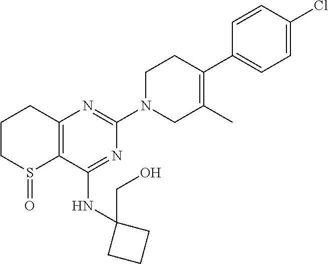 [SMILES: CC1=C(c2ccc(Cl)cc2)CCN(c2nc3c(c(NC4(CO)CCC4)n2)S(=O)CCC3)C1]

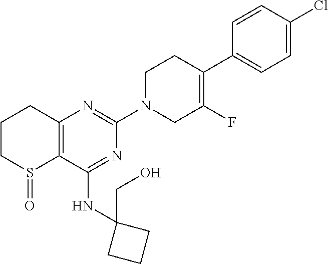 [SMILES: O=S1CCCc2nc(N3CCC(c4ccc(Cl)cc4)=C(F)C3)nc(NC3(CO)CCC3)c21]

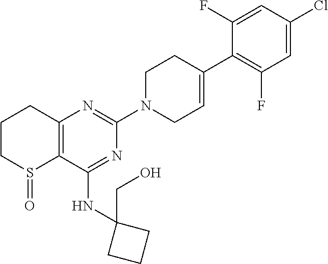 [SMILES: O=S1CCCc2nc(N3CC=C(c4c(F)cc(Cl)cc4F)CC3)nc(NC3(CO)CCC3)c21]

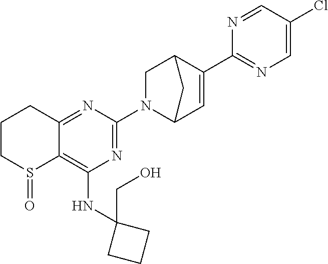 [SMILES: O=S1CCCc2nc(N3CC4CC3C=C4c3ncc(Cl)cn3)nc(NC3(CO)CCC3)c21]

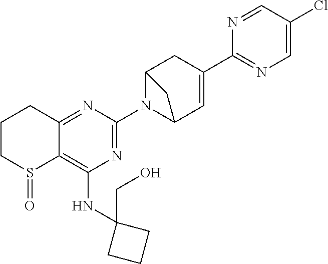 [SMILES: O=S1CCCc2nc(N3C4C=C(c5ncc(Cl)cn5)CC3C4)nc(NC3(CO)CCC3)c21]

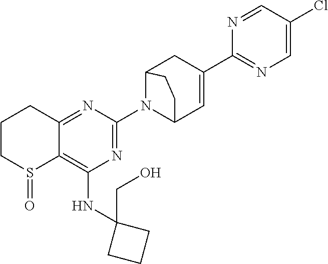 [SMILES: O=S1CCCc2nc(N3C4C=C(c5ncc(Cl)cn5)CC3CC4)nc(NC3(CO)CCC3)c21]

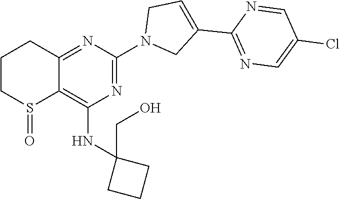 [SMILES: O=S1CCCc2nc(N3CC=C(c4ncc(Cl)cn4)C3)nc(NC3(CO)CCC3)c21]

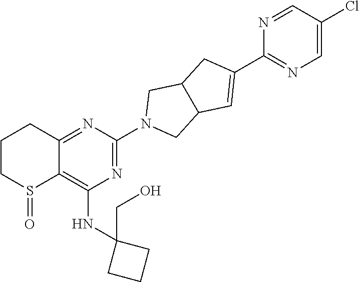 [SMILES: O=S1CCCc2nc(N3CC4C=C(c5ncc(Cl)cn5)CC4C3)nc(NC3(CO)CCC3)c21]

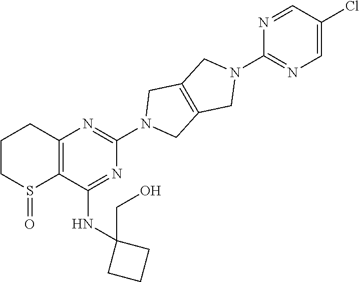 [SMILES: O=S1CCCc2nc(N3CC4=C(CN(c5ncc(Cl)cn5)C4)C3)nc(NC3(CO)CCC3)c21]

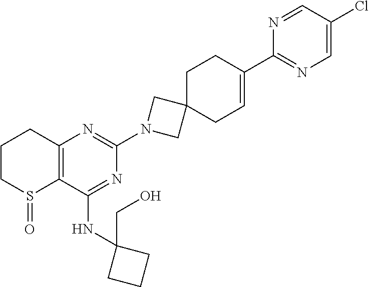 [SMILES: O=S1CCCc2nc(N3CC4(CC=C(c5ncc(Cl)cn5)CC4)C3)nc(NC3(CO)CCC3)c21]

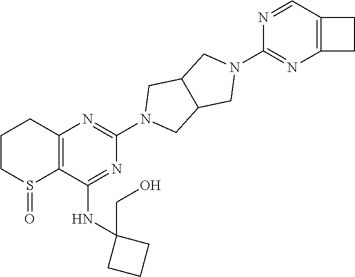 [SMILES: O=S1CCCc2nc(N3CC4CN(c5ncc6c(n5)CC6)CC4C3)nc(NC3(CO)CCC3)c21]

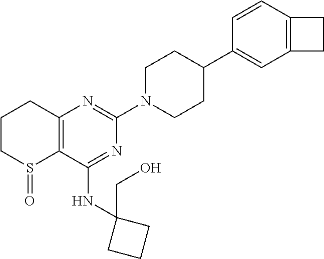 [SMILES: O=S1CCCc2nc(N3CCC(c4ccc5c(c4)CC5)CC3)nc(NC3(CO)CCC3)c21]

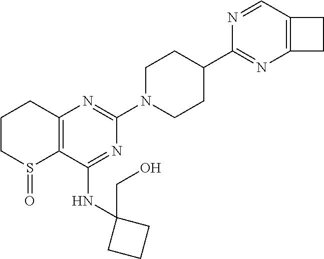 [SMILES: O=S1CCCc2nc(N3CCC(c4ncc5c(n4)CC5)CC3)nc(NC3(CO)CCC3)c21]

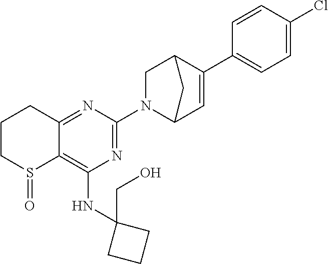 [SMILES: O=S1CCCc2nc(N3CC4CC3C=C4c3ccc(Cl)cc3)nc(NC3(CO)CCC3)c21]

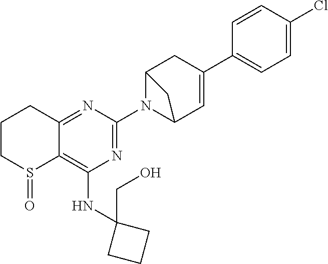 [SMILES: O=S1CCCc2nc(N3C4C=C(c5ccc(Cl)cc5)CC3C4)nc(NC3(CO)CCC3)c21]

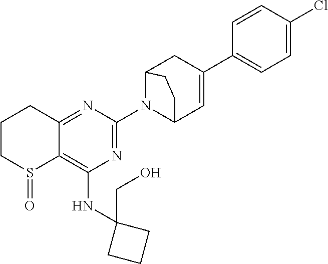 [SMILES: O=S1CCCc2nc(N3C4C=C(c5ccc(Cl)cc5)CC3CC4)nc(NC3(CO)CCC3)c21]

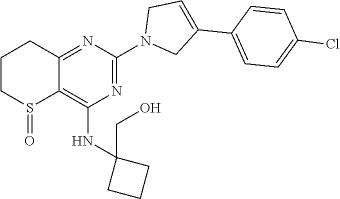 [SMILES: O=S1CCCc2nc(N3CC=C(c4ccc(Cl)cc4)C3)nc(NC3(CO)CCC3)c21]

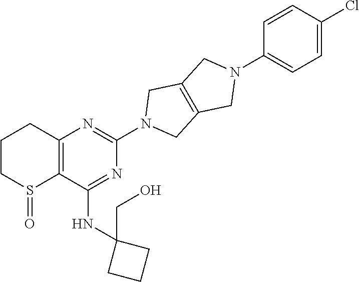 [SMILES: O=S1CCCc2nc(N3CC4=C(CN(c5ccc(Cl)cc5)C4)C3)nc(NC3(CO)CCC3)c21]

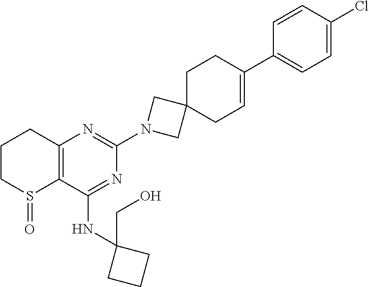 [SMILES: O=S1CCCc2nc(N3CC4(CC=C(c5ccc(Cl)cc5)CC4)C3)nc(NC3(CO)CCC3)c21]

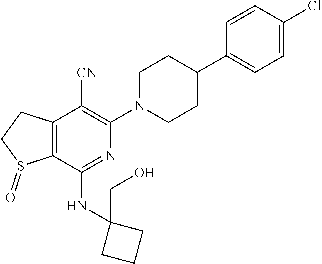 [SMILES: N#Cc1c(N2CCC(c3ccc(Cl)cc3)CC2)nc(NC2(CO)CCC2)c2c1CCS2=O]

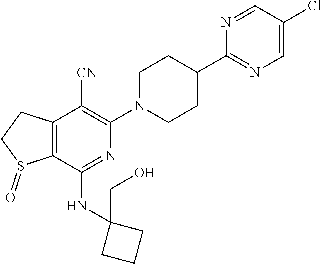 [SMILES: N#Cc1c(N2CCC(c3ncc(Cl)cn3)CC2)nc(NC2(CO)CCC2)c2c1CCS2=O]

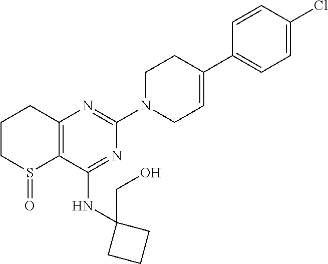 [SMILES: O=S1CCCc2nc(N3CC=C(c4ccc(Cl)cc4)CC3)nc(NC3(CO)CCC3)c21]

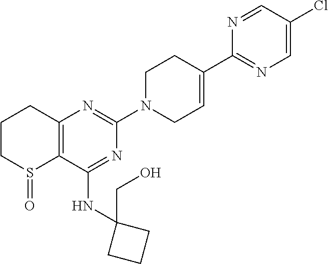 [SMILES: O=S1CCCc2nc(N3CC=C(c4ncc(Cl)cn4)CC3)nc(NC3(CO)CCC3)c21]

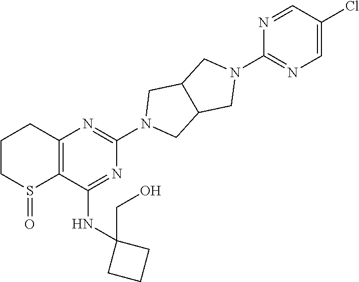 [SMILES: O=S1CCCc2nc(N3CC4CN(c5ncc(Cl)cn5)CC4C3)nc(NC3(CO)CCC3)c21]

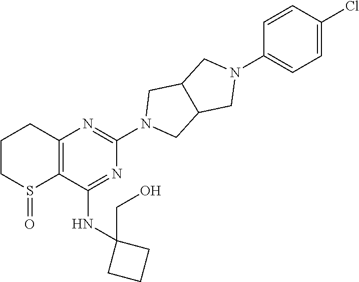 [SMILES: O=S1CCCc2nc(N3CC4CN(c5ccc(Cl)cc5)CC4C3)nc(NC3(CO)CCC3)c21]

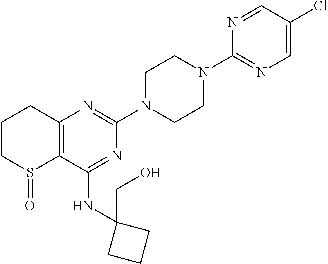 [SMILES: O=S1CCCc2nc(N3CCN(c4ncc(Cl)cn4)CC3)nc(NC3(CO)CCC3)c21]

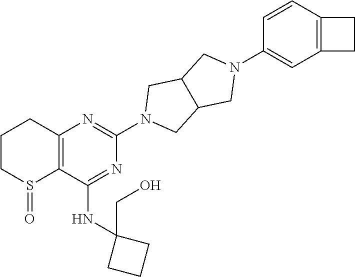 [SMILES: O=S1CCCc2nc(N3CC4CN(c5ccc6c(c5)CC6)CC4C3)nc(NC3(CO)CCC3)c21]

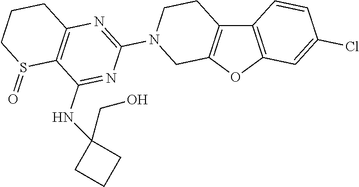 [SMILES: O=S1CCCc2nc(N3CCc4c(oc5cc(Cl)ccc45)C3)nc(NC3(CO)CCC3)c21]

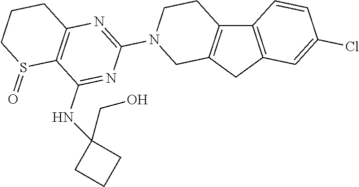 [SMILES: O=S1CCCc2nc(N3CCC4=C(Cc5cc(Cl)ccc54)C3)nc(NC3(CO)CCC3)c21]

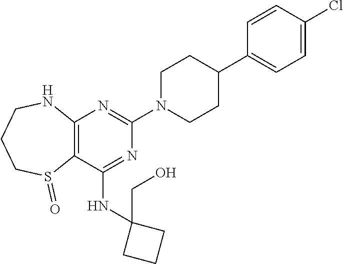 [SMILES: O=S1CCCNc2nc(N3CCC(c4ccc(Cl)cc4)CC3)nc(NC3(CO)CCC3)c21]

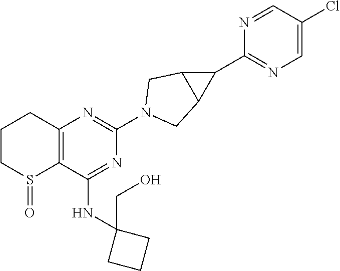 [SMILES: O=S1CCCc2nc(N3CC4C(C3)C4c3ncc(Cl)cn3)nc(NC3(CO)CCC3)c21]

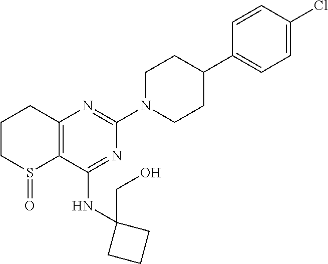 [SMILES: O=S1CCCc2nc(N3CCC(c4ccc(Cl)cc4)CC3)nc(NC3(CO)CCC3)c21]

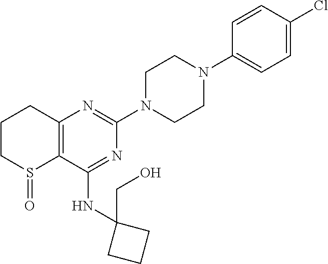 [SMILES: O=S1CCCc2nc(N3CCN(c4ccc(Cl)cc4)CC3)nc(NC3(CO)CCC3)c21]

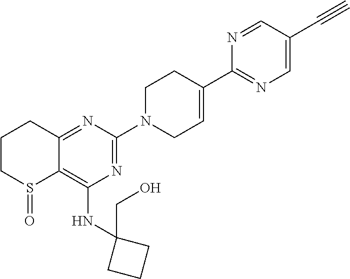 [SMILES: C#Cc1cnc(C2=CCN(c3nc4c(c(NC5(CO)CCC5)n3)S(=O)CCC4)CC2)nc1]

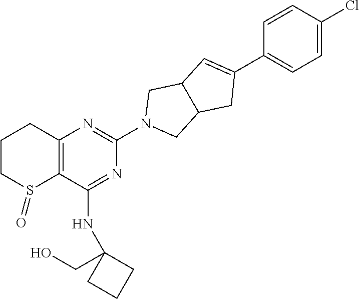 [SMILES: O=S1CCCc2nc(N3CC4C=C(c5ccc(Cl)cc5)CC4C3)nc(NC3(CO)CCC3)c21]

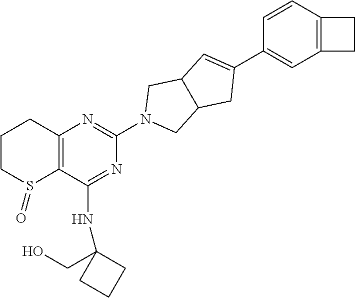 [SMILES: O=S1CCCc2nc(N3CC4C=C(c5ccc6c(c5)CC6)CC4C3)nc(NC3(CO)CCC3)c21]

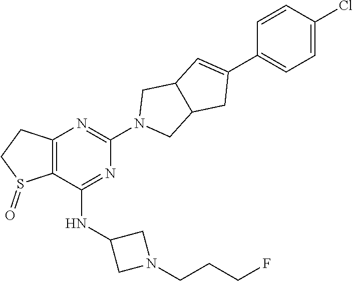 [SMILES: O=S1CCc2nc(N3CC4C=C(c5ccc(Cl)cc5)CC4C3)nc(NC3CN(CCCF)C3)c21]

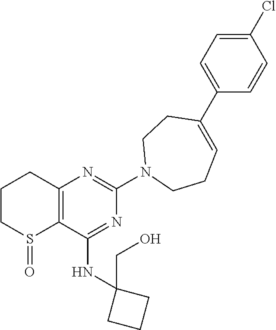 [SMILES: O=S1CCCc2nc(N3CCC=C(c4ccc(Cl)cc4)CC3)nc(NC3(CO)CCC3)c21]

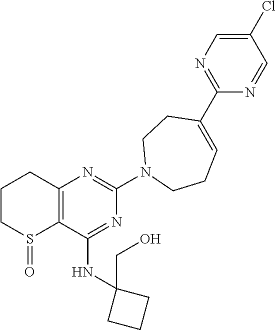 [SMILES: O=S1CCCc2nc(N3CCC=C(c4ncc(Cl)cn4)CC3)nc(NC3(CO)CCC3)c21]

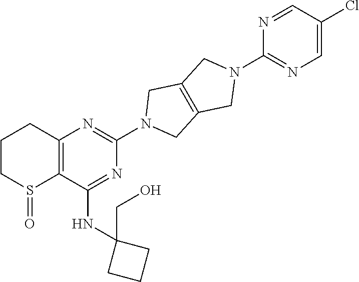 [SMILES: O=S1CCCc2nc(N3CC4=C(CN(c5ncc(Cl)cn5)C4)C3)nc(NC3(CO)CCC3)c21]

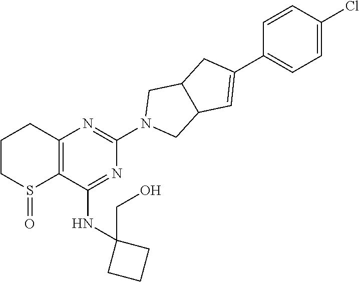 [SMILES: O=S1CCCc2nc(N3CC4C=C(c5ccc(Cl)cc5)CC4C3)nc(NC3(CO)CCC3)c21]

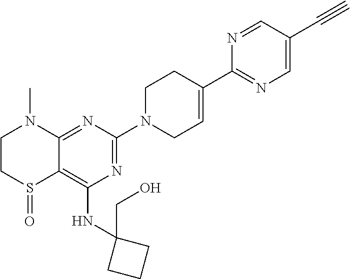 [SMILES: C#Cc1cnc(C2=CCN(c3nc(NC4(CO)CCC4)c4c(n3)N(C)CCS4=O)CC2)nc1]

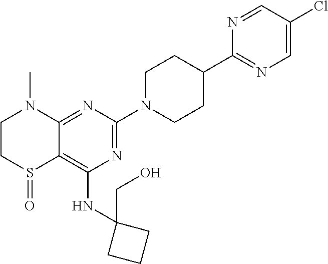 [SMILES: CN1CCS(=O)c2c(NC3(CO)CCC3)nc(N3CCC(c4ncc(Cl)cn4)CC3)nc21]

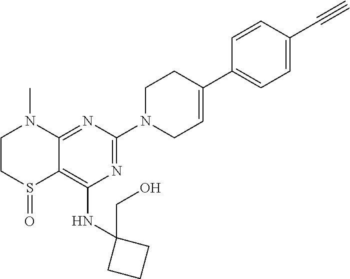 [SMILES: C#Cc1ccc(C2=CCN(c3nc(NC4(CO)CCC4)c4c(n3)N(C)CCS4=O)CC2)cc1]

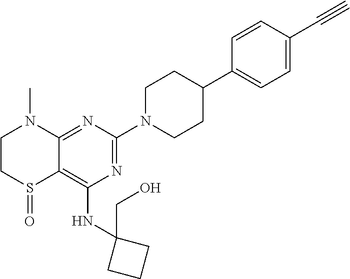 [SMILES: C#Cc1ccc(C2CCN(c3nc(NC4(CO)CCC4)c4c(n3)N(C)CCS4=O)CC2)cc1]

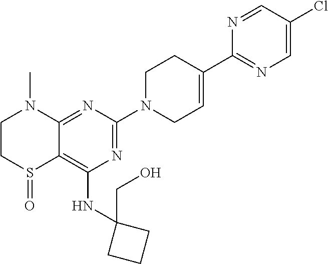 [SMILES: CN1CCS(=O)c2c(NC3(CO)CCC3)nc(N3CC=C(c4ncc(Cl)cn4)CC3)nc21]

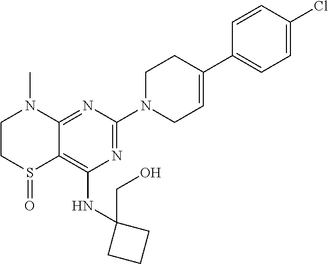 [SMILES: CN1CCS(=O)c2c(NC3(CO)CCC3)nc(N3CC=C(c4ccc(Cl)cc4)CC3)nc21]

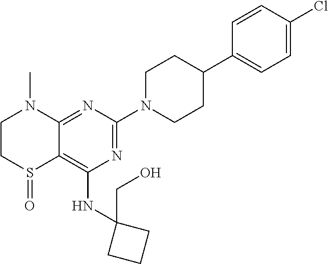 [SMILES: CN1CCS(=O)c2c(NC3(CO)CCC3)nc(N3CCC(c4ccc(Cl)cc4)CC3)nc21]

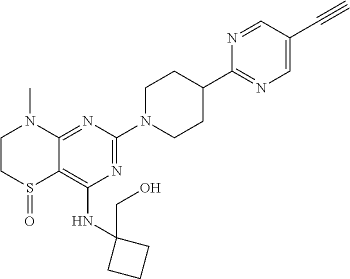 [SMILES: C#Cc1cnc(C2CCN(c3nc(NC4(CO)CCC4)c4c(n3)N(C)CCS4=O)CC2)nc1]

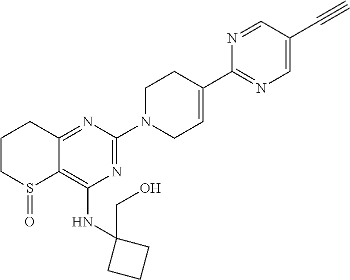 [SMILES: C#Cc1cnc(C2=CCN(c3nc4c(c(NC5(CO)CCC5)n3)S(=O)CCC4)CC2)nc1]

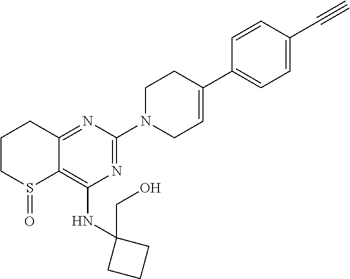 [SMILES: C#Cc1ccc(C2=CCN(c3nc4c(c(NC5(CO)CCC5)n3)S(=O)CCC4)CC2)cc1]

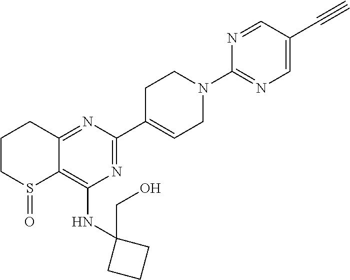 [SMILES: C#Cc1cnc(N2CC=C(c3nc4c(c(NC5(CO)CCC5)n3)S(=O)CCC4)CC2)nc1]

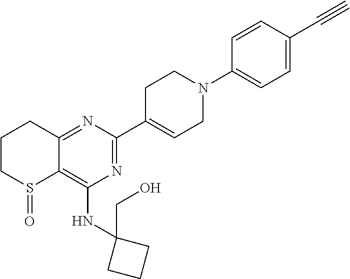 [SMILES: C#Cc1ccc(N2CC=C(c3nc4c(c(NC5(CO)CCC5)n3)S(=O)CCC4)CC2)cc1]

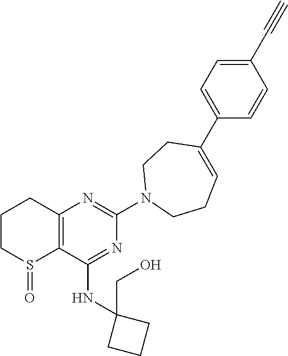 [SMILES: C#Cc1ccc(C2=CCCN(c3nc4c(c(NC5(CO)CCC5)n3)S(=O)CCC4)CC2)cc1]

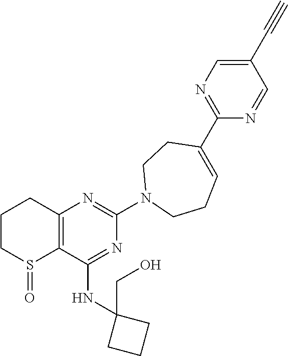 [SMILES: C#Cc1cnc(C2=CCCN(c3nc4c(c(NC5(CO)CCC5)n3)S(=O)CCC4)CC2)nc1]

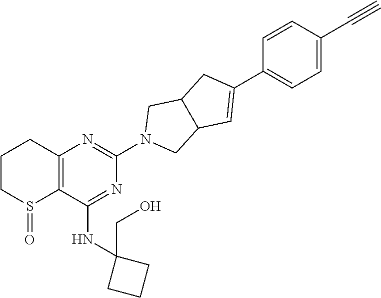 [SMILES: C#Cc1ccc(C2=CC3CN(c4nc5c(c(NC6(CO)CCC6)n4)S(=O)CCC5)CC3C2)cc1]

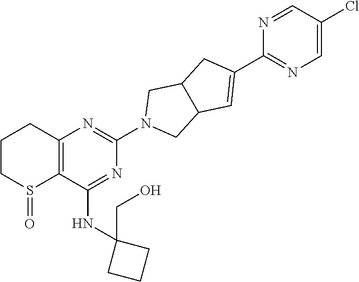 [SMILES: O=S1CCCc2nc(N3CC4C=C(c5ncc(Cl)cn5)CC4C3)nc(NC3(CO)CCC3)c21]

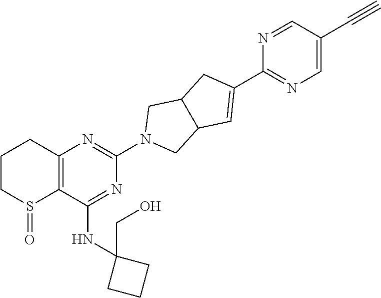 [SMILES: C#Cc1cnc(C2=CC3CN(c4nc5c(c(NC6(CO)CCC6)n4)S(=O)CCC5)CC3C2)nc1]

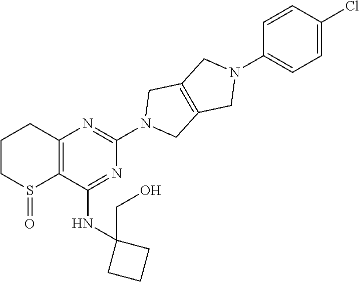 [SMILES: O=S1CCCc2nc(N3CC4=C(CN(c5ccc(Cl)cc5)C4)C3)nc(NC3(CO)CCC3)c21]

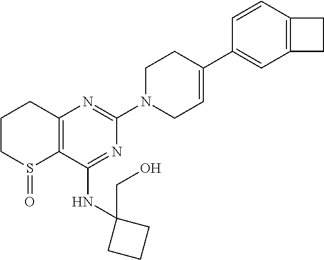 [SMILES: O=S1CCCc2nc(N3CC=C(c4ccc5c(c4)CC5)CC3)nc(NC3(CO)CCC3)c21]

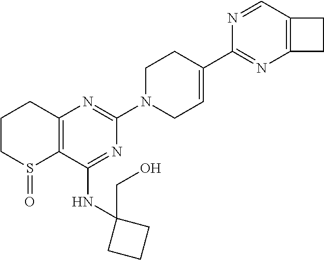 [SMILES: O=S1CCCc2nc(N3CC=C(c4ncc5c(n4)CC5)CC3)nc(NC3(CO)CCC3)c21]

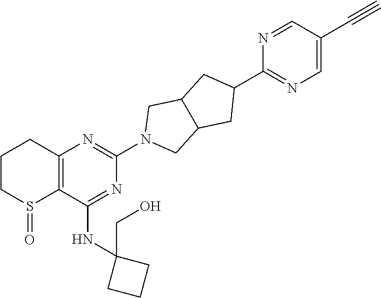 [SMILES: C#Cc1cnc(C2CC3CN(c4nc5c(c(NC6(CO)CCC6)n4)S(=O)CCC5)CC3C2)nc1]

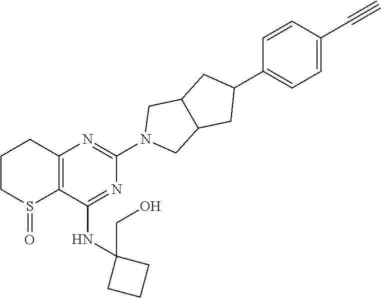 [SMILES: C#Cc1ccc(C2CC3CN(c4nc5c(c(NC6(CO)CCC6)n4)S(=O)CCC5)CC3C2)cc1]

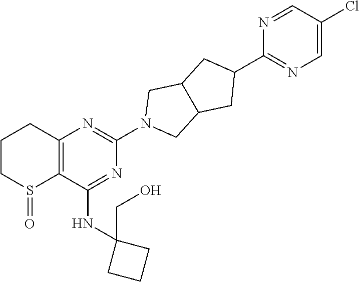 [SMILES: O=S1CCCc2nc(N3CC4CC(c5ncc(Cl)cn5)CC4C3)nc(NC3(CO)CCC3)c21]

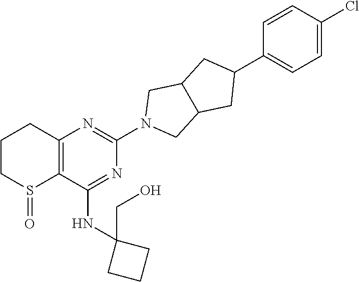 [SMILES: O=S1CCCc2nc(N3CC4CC(c5ccc(Cl)cc5)CC4C3)nc(NC3(CO)CCC3)c21]

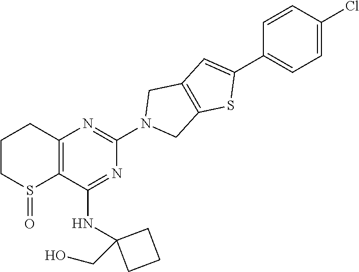 [SMILES: O=S1CCCc2nc(N3Cc4cc(-c5ccc(Cl)cc5)sc4C3)nc(NC3(CO)CCC3)c21]

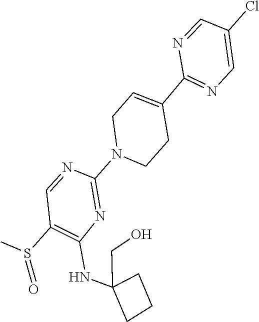 [SMILES: CS(=O)c1cnc(N2CC=C(c3ncc(Cl)cn3)CC2)nc1NC1(CO)CCC1]

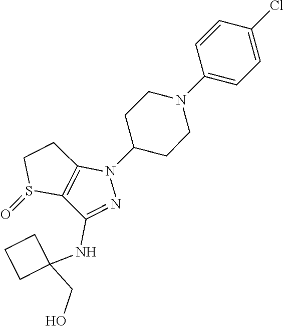 [SMILES: O=S1CCc2c1c(NC1(CO)CCC1)nn2C1CCN(c2ccc(Cl)cc2)CC1]

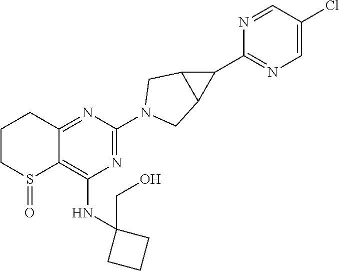 [SMILES: O=S1CCCc2nc(N3CC4C(C3)C4c3ncc(Cl)cn3)nc(NC3(CO)CCC3)c21]

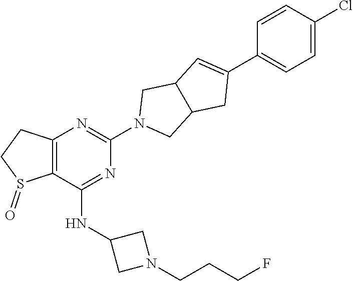 [SMILES: O=S1CCc2nc(N3CC4C=C(c5ccc(Cl)cc5)CC4C3)nc(NC3CN(CCCF)C3)c21]

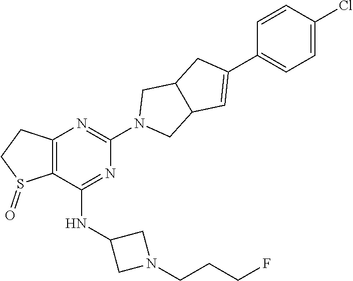 [SMILES: O=S1CCc2nc(N3CC4C=C(c5ccc(Cl)cc5)CC4C3)nc(NC3CN(CCCF)C3)c21]

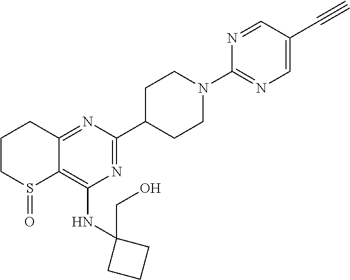 [SMILES: C#Cc1cnc(N2CCC(c3nc4c(c(NC5(CO)CCC5)n3)S(=O)CCC4)CC2)nc1]

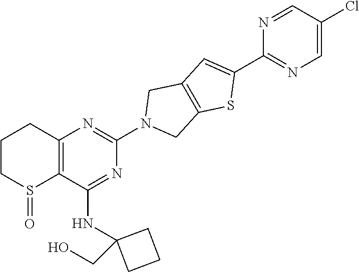 [SMILES: O=S1CCCc2nc(N3Cc4cc(-c5ncc(Cl)cn5)sc4C3)nc(NC3(CO)CCC3)c21]

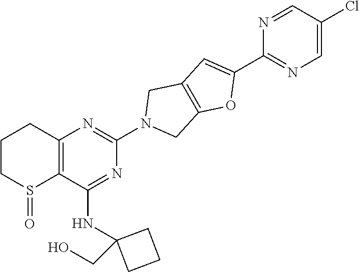 [SMILES: O=S1CCCc2nc(N3Cc4cc(-c5ncc(Cl)cn5)oc4C3)nc(NC3(CO)CCC3)c21]

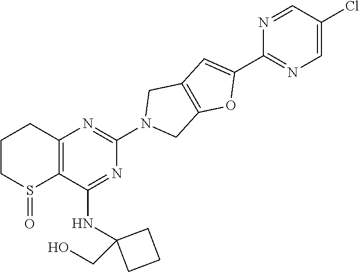 [SMILES: O=S1CCCc2nc(N3Cc4cc(-c5ncc(Cl)cn5)oc4C3)nc(NC3(CO)CCC3)c21]

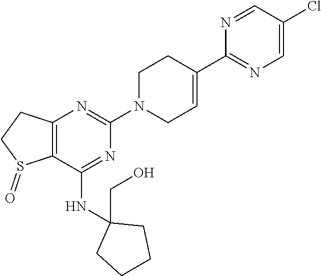 [SMILES: O=S1CCc2nc(N3CC=C(c4ncc(Cl)cn4)CC3)nc(NC3(CO)CCCC3)c21]

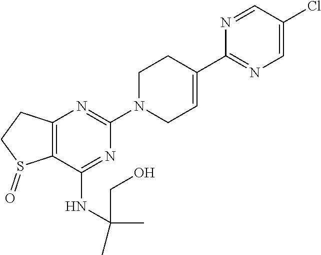 [SMILES: CC(C)(CO)Nc1nc(N2CC=C(c3ncc(Cl)cn3)CC2)nc2c1S(=O)CC2]

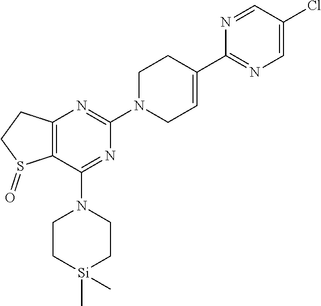 [SMILES: C[Si]1(C)CCN(c2nc(N3CC=C(c4ncc(Cl)cn4)CC3)nc3c2S(=O)CC3)CC1]

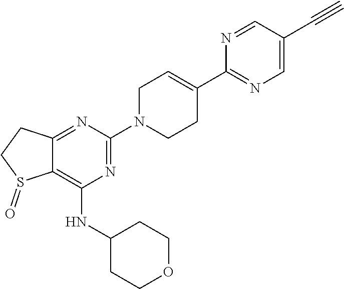 [SMILES: C#Cc1cnc(C2=CCN(c3nc4c(c(NC5CCOCC5)n3)S(=O)CC4)CC2)nc1]

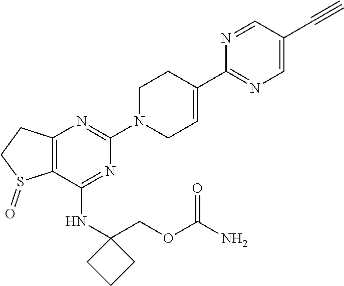 [SMILES: C#Cc1cnc(C2=CCN(c3nc4c(c(NC5(COC(N)=O)CCC5)n3)S(=O)CC4)CC2)nc1]

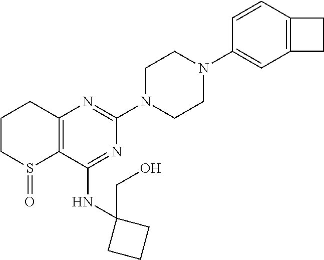 [SMILES: O=S1CCCc2nc(N3CCN(c4ccc5c(c4)CC5)CC3)nc(NC3(CO)CCC3)c21]

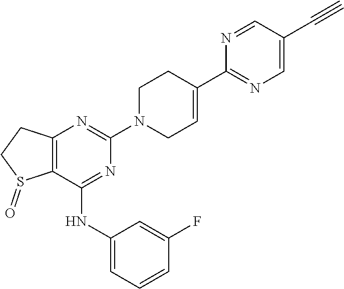 [SMILES: C#Cc1cnc(C2=CCN(c3nc4c(c(Nc5cccc(F)c5)n3)S(=O)CC4)CC2)nc1]

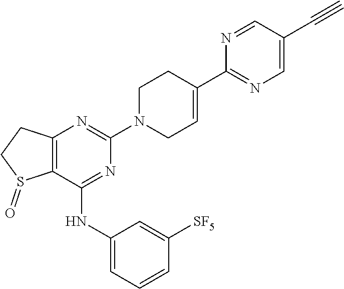

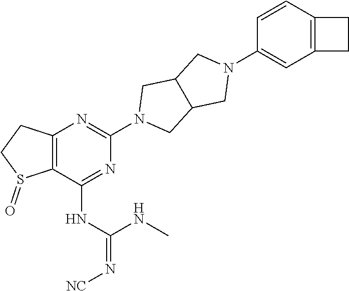 [SMILES: CN/C(=N/C#N)Nc1nc(N2CC3CN(c4ccc5c(c4)CC5)CC3C2)nc2c1S(=O)CC2]

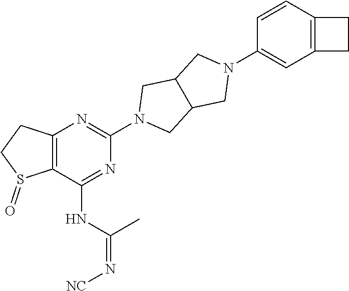 [SMILES: C/C(=N/C#N)Nc1nc(N2CC3CN(c4ccc5c(c4)CC5)CC3C2)nc2c1S(=O)CC2]

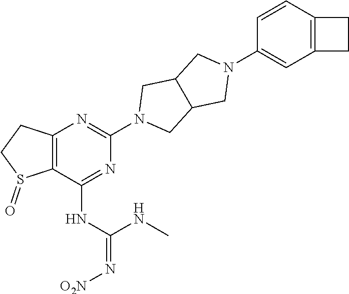 [SMILES: CN/C(=N/[N+](=O)[O-])Nc1nc(N2CC3CN(c4ccc5c(c4)CC5)CC3C2)nc2c1S(=O)CC2]

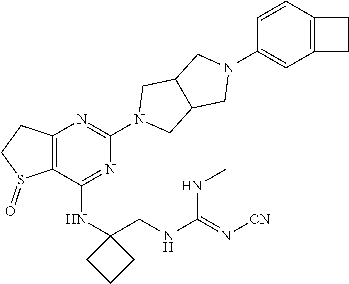 [SMILES: CN/C(=N\C#N)NCC1(Nc2nc(N3CC4CN(c5ccc6c(c5)CC6)CC4C3)nc3c2S(=O)CC3)CCC1]

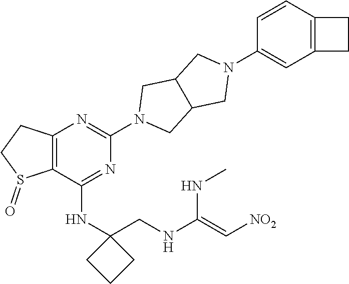 [SMILES: CN/C(=C\[N+](=O)[O-])NCC1(Nc2nc(N3CC4CN(c5ccc6c(c5)CC6)CC4C3)nc3c2S(=O)CC3)CCC1]

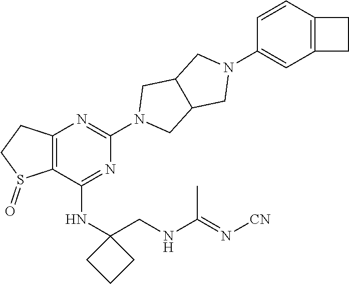 [SMILES: C/C(=N\C#N)NCC1(Nc2nc(N3CC4CN(c5ccc6c(c5)CC6)CC4C3)nc3c2S(=O)CC3)CCC1]

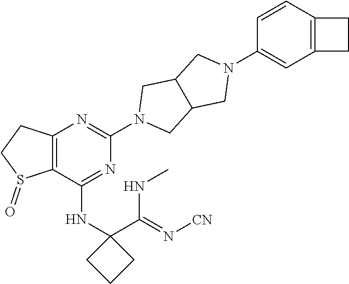 [SMILES: CN/C(=N\C#N)C1(Nc2nc(N3CC4CN(c5ccc6c(c5)CC6)CC4C3)nc3c2S(=O)CC3)CCC1]

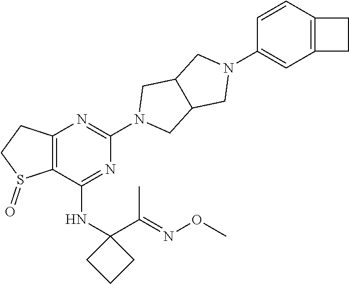 [SMILES: CO/N=C(\C)C1(Nc2nc(N3CC4CN(c5ccc6c(c5)CC6)CC4C3)nc3c2S(=O)CC3)CCC1]

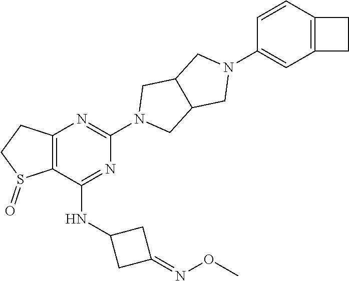 [SMILES: CON=C1CC(Nc2nc(N3CC4CN(c5ccc6c(c5)CC6)CC4C3)nc3c2S(=O)CC3)C1]

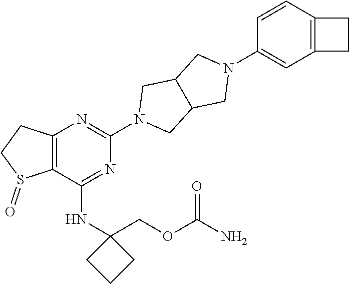 [SMILES: NC(=O)OCC1(Nc2nc(N3CC4CN(c5ccc6c(c5)CC6)CC4C3)nc3c2S(=O)CC3)CCC1]

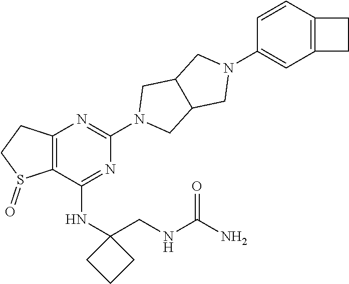 [SMILES: NC(=O)NCC1(Nc2nc(N3CC4CN(c5ccc6c(c5)CC6)CC4C3)nc3c2S(=O)CC3)CCC1]

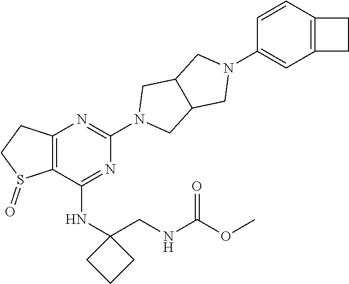 [SMILES: COC(=O)NCC1(Nc2nc(N3CC4CN(c5ccc6c(c5)CC6)CC4C3)nc3c2S(=O)CC3)CCC1]

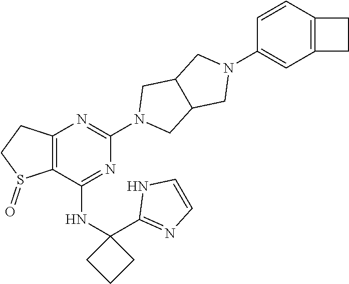 [SMILES: O=S1CCc2nc(N3CC4CN(c5ccc6c(c5)CC6)CC4C3)nc(NC3(c4ncc[nH]4)CCC3)c21]

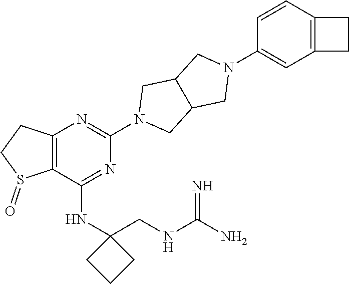 [SMILES: N=C(N)NCC1(Nc2nc(N3CC4CN(c5ccc6c(c5)CC6)CC4C3)nc3c2S(=O)CC3)CCC1]

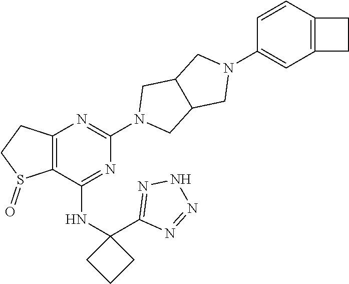 [SMILES: O=S1CCc2nc(N3CC4CN(c5ccc6c(c5)CC6)CC4C3)nc(NC3(c4nn[nH]n4)CCC3)c21]

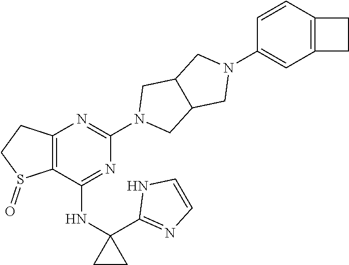 [SMILES: O=S1CCc2nc(N3CC4CN(c5ccc6c(c5)CC6)CC4C3)nc(NC3(c4ncc[nH]4)CC3)c21]

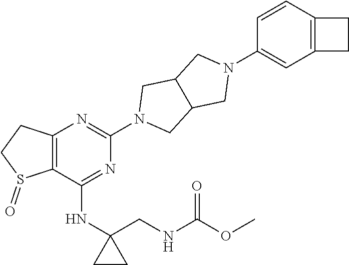 [SMILES: COC(=O)NCC1(Nc2nc(N3CC4CN(c5ccc6c(c5)CC6)CC4C3)nc3c2S(=O)CC3)CC1]

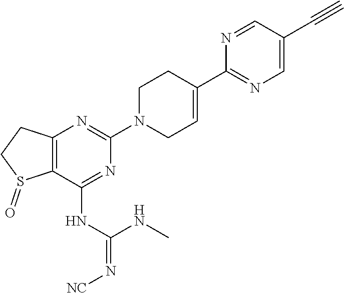 [SMILES: C#Cc1cnc(C2=CCN(c3nc4c(c(N/C(=N\C#N)NC)n3)S(=O)CC4)CC2)nc1]

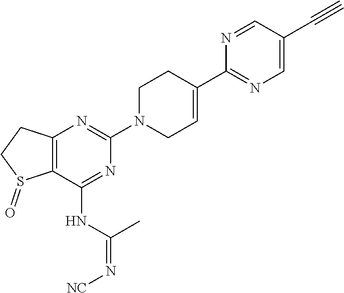 [SMILES: C#Cc1cnc(C2=CCN(c3nc4c(c(N/C(C)=N\C#N)n3)S(=O)CC4)CC2)nc1]

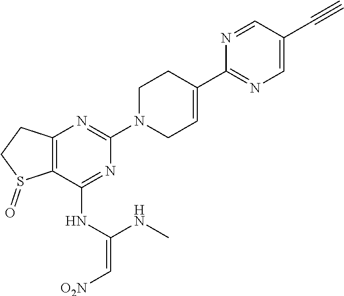 [SMILES: C#Cc1cnc(C2=CCN(c3nc4c(c(N/C(=C\[N+](=O)[O-])NC)n3)S(=O)CC4)CC2)nc1]

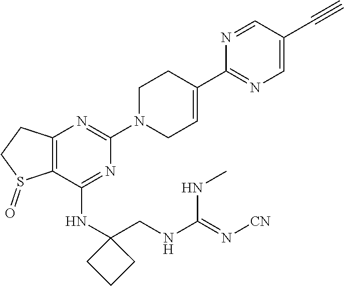 [SMILES: C#Cc1cnc(C2=CCN(c3nc4c(c(NC5(CN/C(=N/C#N)NC)CCC5)n3)S(=O)CC4)CC2)nc1]

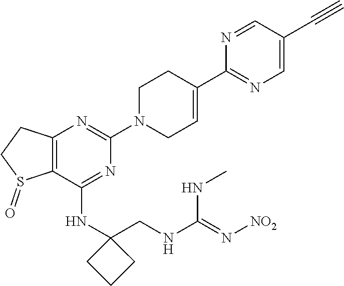 [SMILES: C#Cc1cnc(C2=CCN(c3nc4c(c(NC5(CN/C(=N/[N+](=O)[O-])NC)CCC5)n3)S(=O)CC4)CC2)nc1]

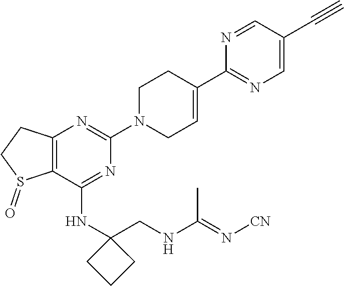 [SMILES: C#Cc1cnc(C2=CCN(c3nc4c(c(NC5(CN/C(C)=N/C#N)CCC5)n3)S(=O)CC4)CC2)nc1]

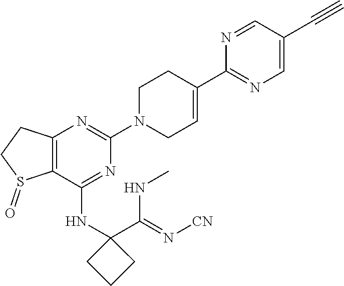 [SMILES: C#Cc1cnc(C2=CCN(c3nc4c(c(NC5(/C(=N/C#N)NC)CCC5)n3)S(=O)CC4)CC2)nc1]

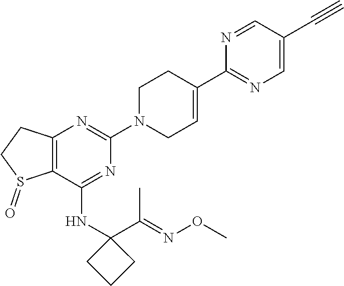 [SMILES: C#Cc1cnc(C2=CCN(c3nc4c(c(NC5(/C(C)=N/OC)CCC5)n3)S(=O)CC4)CC2)nc1]

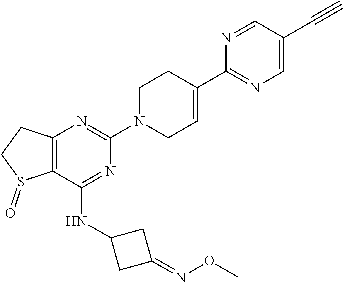 [SMILES: C#Cc1cnc(C2=CCN(c3nc4c(c(NC5CC(=NOC)C5)n3)S(=O)CC4)CC2)nc1]

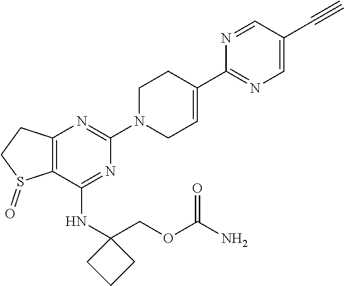 [SMILES: C#Cc1cnc(C2=CCN(c3nc4c(c(NC5(COC(N)=O)CCC5)n3)S(=O)CC4)CC2)nc1]

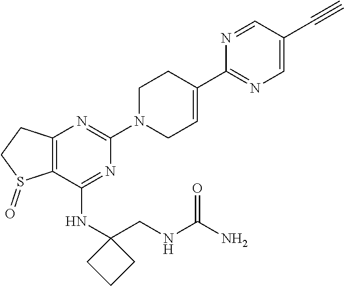 [SMILES: C#Cc1cnc(C2=CCN(c3nc4c(c(NC5(CNC(N)=O)CCC5)n3)S(=O)CC4)CC2)nc1]

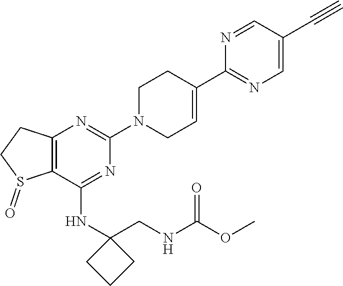 [SMILES: C#Cc1cnc(C2=CCN(c3nc4c(c(NC5(CNC(=O)OC)CCC5)n3)S(=O)CC4)CC2)nc1]

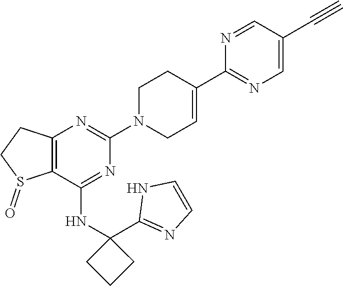 [SMILES: C#Cc1cnc(C2=CCN(c3nc4c(c(NC5(c6ncc[nH]6)CCC5)n3)S(=O)CC4)CC2)nc1]

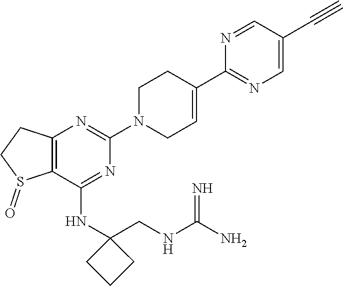 [SMILES: C#Cc1cnc(C2=CCN(c3nc4c(c(NC5(CNC(=N)N)CCC5)n3)S(=O)CC4)CC2)nc1]

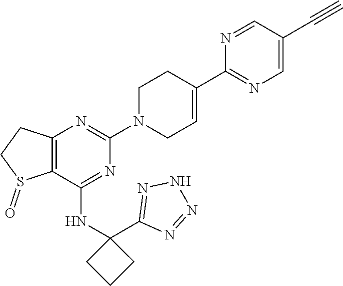 [SMILES: C#Cc1cnc(C2=CCN(c3nc4c(c(NC5(c6nn[nH]n6)CCC5)n3)S(=O)CC4)CC2)nc1]

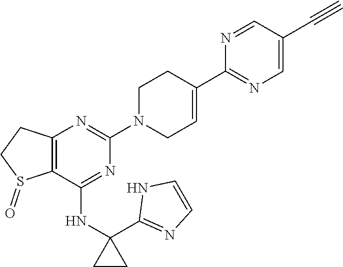 [SMILES: C#Cc1cnc(C2=CCN(c3nc4c(c(NC5(c6ncc[nH]6)CC5)n3)S(=O)CC4)CC2)nc1]

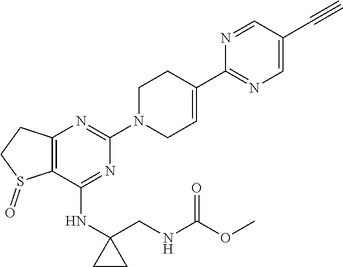 [SMILES: C#Cc1cnc(C2=CCN(c3nc4c(c(NC5(CNC(=O)OC)CC5)n3)S(=O)CC4)CC2)nc1]

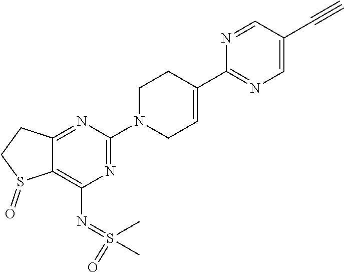 [SMILES: C#Cc1cnc(C2=CCN(c3nc4c(c(N=S(C)(C)=O)n3)S(=O)CC4)CC2)nc1]

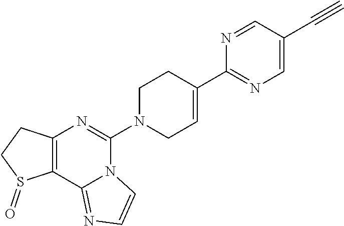 [SMILES: C#Cc1cnc(C2=CCN(c3nc4c(c5nccn35)S(=O)CC4)CC2)nc1]

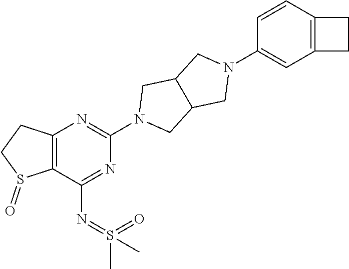 [SMILES: CS(C)(=O)=Nc1nc(N2CC3CN(c4ccc5c(c4)CC5)CC3C2)nc2c1S(=O)CC2]

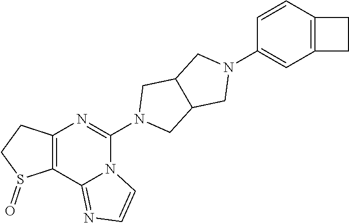 [SMILES: O=S1CCc2nc(N3CC4CN(c5ccc6c(c5)CC6)CC4C3)n3ccnc3c21]

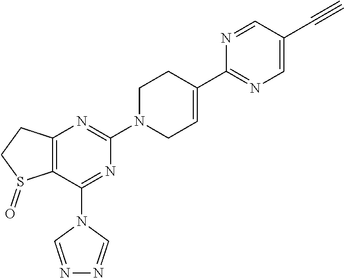 [SMILES: C#Cc1cnc(C2=CCN(c3nc4c(c(-n5cnnc5)n3)S(=O)CC4)CC2)nc1]

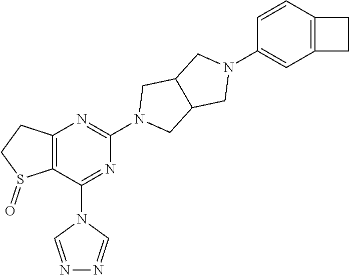 [SMILES: O=S1CCc2nc(N3CC4CN(c5ccc6c(c5)CC6)CC4C3)nc(-n3cnnc3)c21]

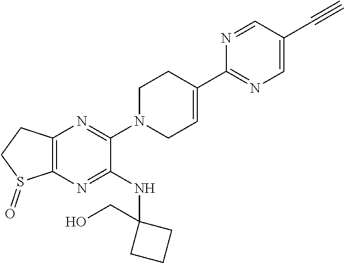 [SMILES: C#Cc1cnc(C2=CCN(c3nc4c(nc3NC3(CO)CCC3)S(=O)CC4)CC2)nc1]

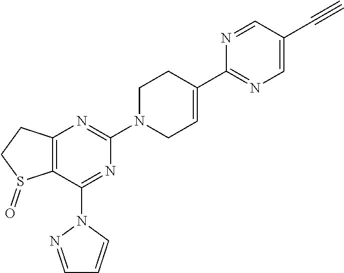 [SMILES: C#Cc1cnc(C2=CCN(c3nc4c(c(-n5cccn5)n3)S(=O)CC4)CC2)nc1]

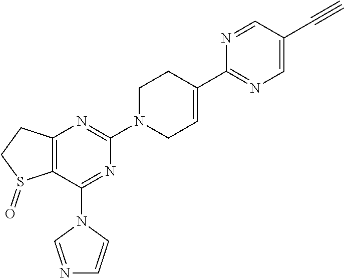 [SMILES: C#Cc1cnc(C2=CCN(c3nc4c(c(-n5ccnc5)n3)S(=O)CC4)CC2)nc1]

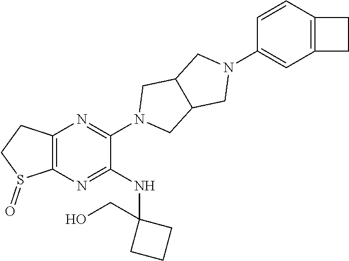 [SMILES: O=S1CCc2nc(N3CC4CN(c5ccc6c(c5)CC6)CC4C3)c(NC3(CO)CCC3)nc21]

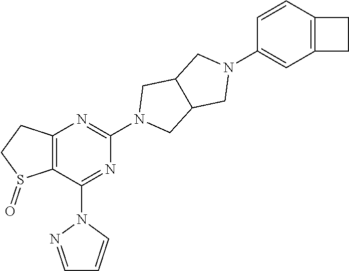 [SMILES: O=S1CCc2nc(N3CC4CN(c5ccc6c(c5)CC6)CC4C3)nc(-n3cccn3)c21]

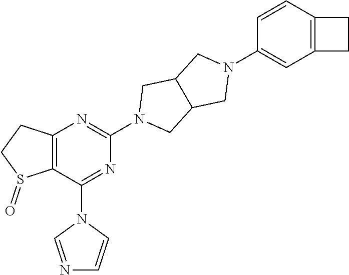 [SMILES: O=S1CCc2nc(N3CC4CN(c5ccc6c(c5)CC6)CC4C3)nc(-n3ccnc3)c21]

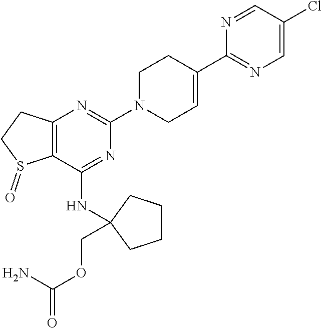 [SMILES: NC(=O)OCC1(Nc2nc(N3CC=C(c4ncc(Cl)cn4)CC3)nc3c2S(=O)CC3)CCCC1]

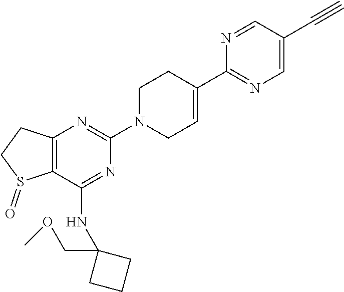 [SMILES: C#Cc1cnc(C2=CCN(c3nc4c(c(NC5(COC)CCC5)n3)S(=O)CC4)CC2)nc1]

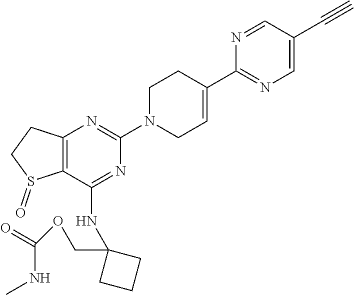 [SMILES: C#Cc1cnc(C2=CCN(c3nc4c(c(NC5(COC(=O)NC)CCC5)n3)S(=O)CC4)CC2)nc1]

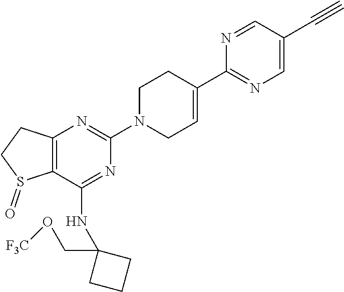 [SMILES: C#Cc1cnc(C2=CCN(c3nc4c(c(NC5(COC(F)(F)F)CCC5)n3)S(=O)CC4)CC2)nc1]

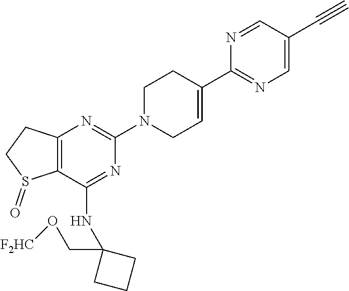 [SMILES: C#Cc1cnc(C2=CCN(c3nc4c(c(NC5(COC(F)F)CCC5)n3)S(=O)CC4)CC2)nc1]

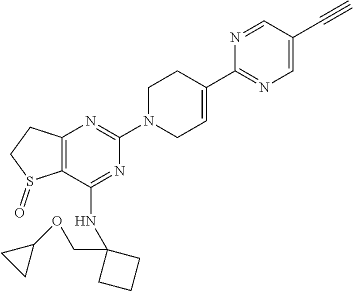 [SMILES: C#Cc1cnc(C2=CCN(c3nc4c(c(NC5(COC6CC6)CCC5)n3)S(=O)CC4)CC2)nc1]

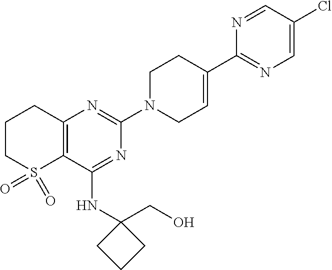 [SMILES: O=S1(=O)CCCc2nc(N3CC=C(c4ncc(Cl)cn4)CC3)nc(NC3(CO)CCC3)c21]

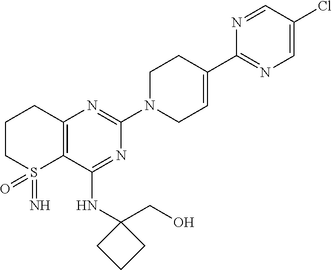 [SMILES: N=S1(=O)CCCc2nc(N3CC=C(c4ncc(Cl)cn4)CC3)nc(NC3(CO)CCC3)c21]

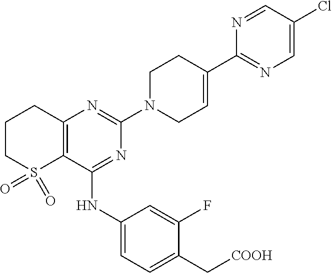 [SMILES: O=C(O)Cc1ccc(Nc2nc(N3CC=C(c4ncc(Cl)cn4)CC3)nc3c2S(=O)(=O)CCC3)cc1F]

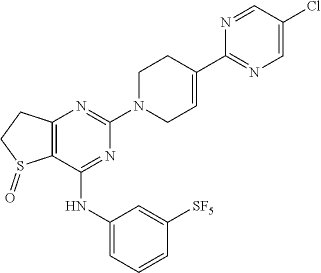

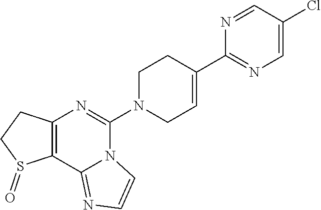 [SMILES: O=S1CCc2nc(N3CC=C(c4ncc(Cl)cn4)CC3)n3ccnc3c21]

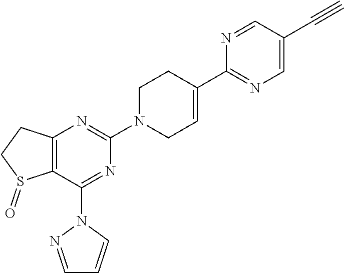 [SMILES: C#Cc1cnc(C2=CCN(c3nc4c(c(-n5cccn5)n3)S(=O)CC4)CC2)nc1]

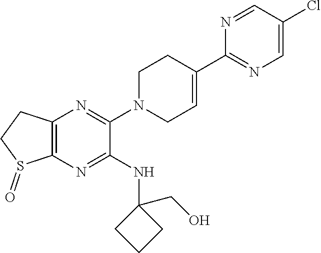 [SMILES: O=S1CCc2nc(N3CC=C(c4ncc(Cl)cn4)CC3)c(NC3(CO)CCC3)nc21]

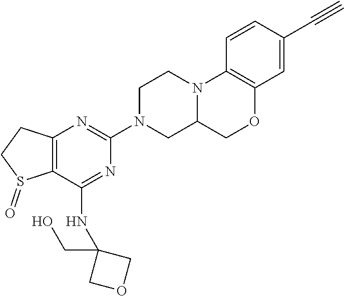 [SMILES: C#Cc1ccc2c(c1)OCC1CN(c3nc4c(c(NC5(CO)COC5)n3)S(=O)CC4)CCN21]

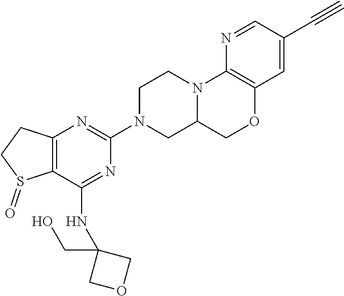 [SMILES: C#Cc1cnc2c(c1)OCC1CN(c3nc4c(c(NC5(CO)COC5)n3)S(=O)CC4)CCN21]

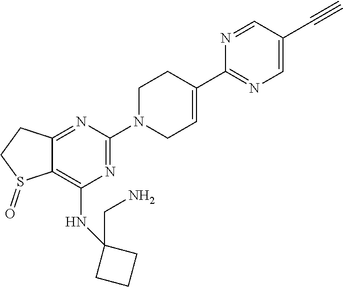 [SMILES: C#Cc1cnc(C2=CCN(c3nc4c(c(NC5(CN)CCC5)n3)S(=O)CC4)CC2)nc1]

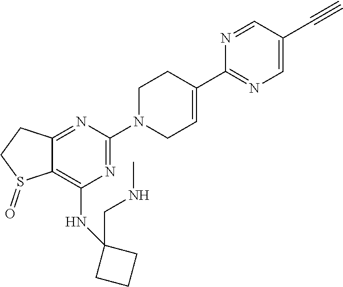 [SMILES: C#Cc1cnc(C2=CCN(c3nc4c(c(NC5(CNC)CCC5)n3)S(=O)CC4)CC2)nc1]

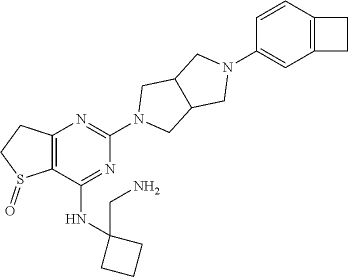 [SMILES: NCC1(Nc2nc(N3CC4CN(c5ccc6c(c5)CC6)CC4C3)nc3c2S(=O)CC3)CCC1]

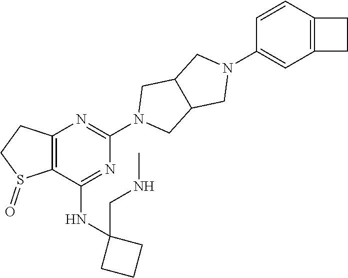 [SMILES: CNCC1(Nc2nc(N3CC4CN(c5ccc6c(c5)CC6)CC4C3)nc3c2S(=O)CC3)CCC1]

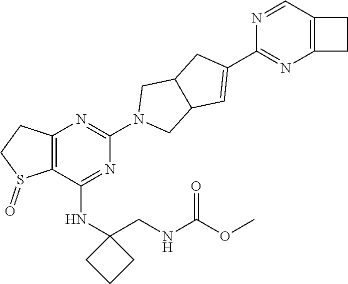 [SMILES: COC(=O)NCC1(Nc2nc(N3CC4C=C(c5ncc6c(n5)CC6)CC4C3)nc3c2S(=O)CC3)CCC1]

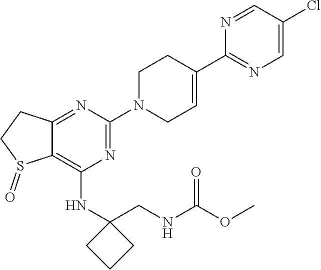 [SMILES: COC(=O)NCC1(Nc2nc(N3CC=C(c4ncc(Cl)cn4)CC3)nc3c2S(=O)CC3)CCC1]

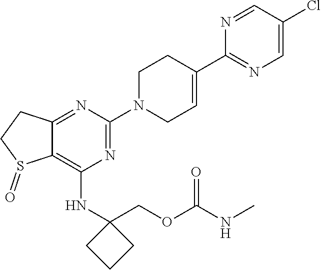 [SMILES: CNC(=O)OCC1(Nc2nc(N3CC=C(c4ncc(Cl)cn4)CC3)nc3c2S(=O)CC3)CCC1]

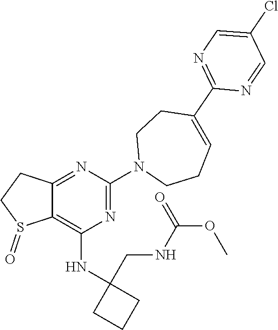 [SMILES: COC(=O)NCC1(Nc2nc(N3CCC=C(c4ncc(Cl)cn4)CC3)nc3c2S(=O)CC3)CCC1]

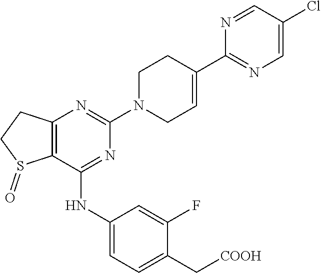 [SMILES: O=C(O)Cc1ccc(Nc2nc(N3CC=C(c4ncc(Cl)cn4)CC3)nc3c2S(=O)CC3)cc1F]

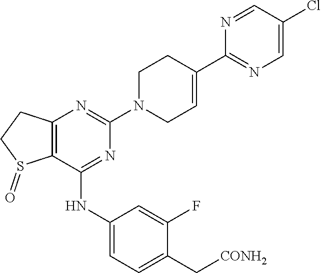 [SMILES: NC(=O)Cc1ccc(Nc2nc(N3CC=C(c4ncc(Cl)cn4)CC3)nc3c2S(=O)CC3)cc1F]

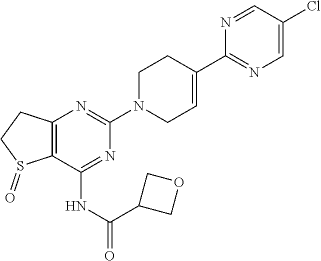 [SMILES: O=C(Nc1nc(N2CC=C(c3ncc(Cl)cn3)CC2)nc2c1S(=O)CC2)C1COC1]

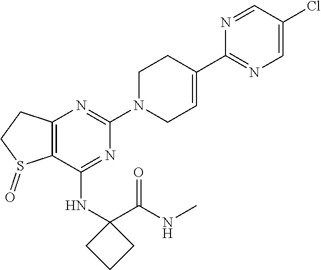 [SMILES: CNC(=O)C1(Nc2nc(N3CC=C(c4ncc(Cl)cn4)CC3)nc3c2S(=O)CC3)CCC1]

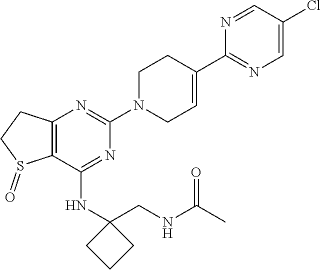 [SMILES: CC(=O)NCC1(Nc2nc(N3CC=C(c4ncc(Cl)cn4)CC3)nc3c2S(=O)CC3)CCC1]

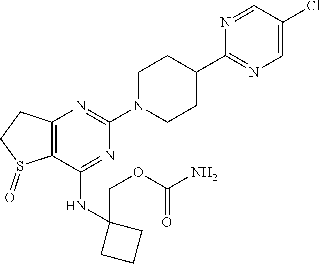 [SMILES: NC(=O)OCC1(Nc2nc(N3CCC(c4ncc(Cl)cn4)CC3)nc3c2S(=O)CC3)CCC1]

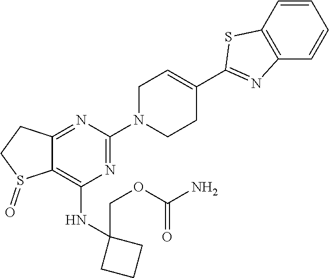 [SMILES: NC(=O)OCC1(Nc2nc(N3CC=C(c4nc5ccccc5s4)CC3)nc3c2S(=O)CC3)CCC1]

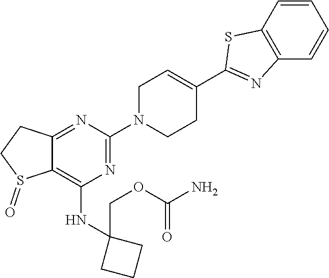 [SMILES: NC(=O)OCC1(Nc2nc(N3CC=C(c4nc5ccccc5s4)CC3)nc3c2S(=O)CC3)CCC1]

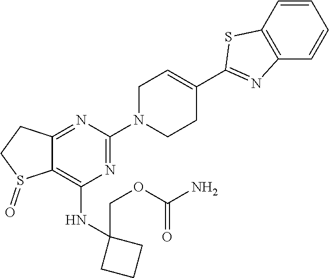 [SMILES: NC(=O)OCC1(Nc2nc(N3CC=C(c4nc5ccccc5s4)CC3)nc3c2S(=O)CC3)CCC1]

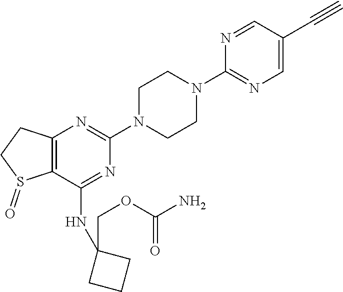 [SMILES: C#Cc1cnc(N2CCN(c3nc4c(c(NC5(COC(N)=O)CCC5)n3)S(=O)CC4)CC2)nc1]

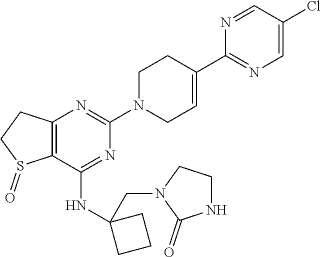 [SMILES: O=C1NCCN1CC1(Nc2nc(N3CC=C(c4ncc(Cl)cn4)CC3)nc3c2S(=O)CC3)CCC1]

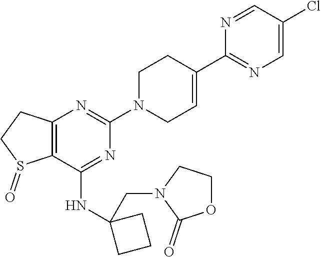 [SMILES: O=C1OCCN1CC1(Nc2nc(N3CC=C(c4ncc(Cl)cn4)CC3)nc3c2S(=O)CC3)CCC1]

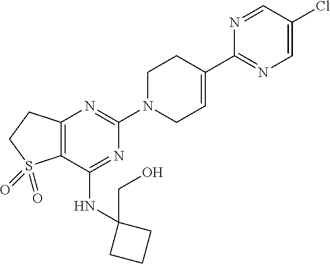 [SMILES: O=S1(=O)CCc2nc(N3CC=C(c4ncc(Cl)cn4)CC3)nc(NC3(CO)CCC3)c21]

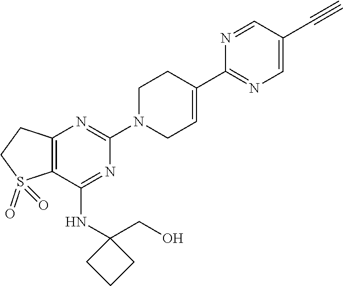 [SMILES: C#Cc1cnc(C2=CCN(c3nc4c(c(NC5(CO)CCC5)n3)S(=O)(=O)CC4)CC2)nc1]

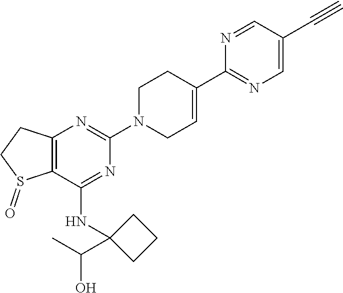 [SMILES: C#Cc1cnc(C2=CCN(c3nc4c(c(NC5(C(C)O)CCC5)n3)S(=O)CC4)CC2)nc1]

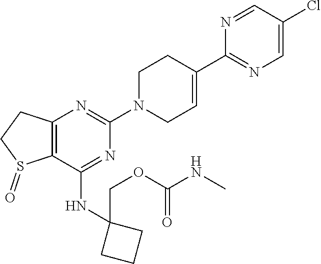 [SMILES: CNC(=O)OCC1(Nc2nc(N3CC=C(c4ncc(Cl)cn4)CC3)nc3c2S(=O)CC3)CCC1]

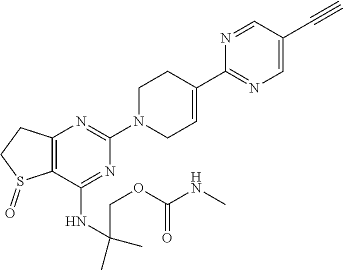 [SMILES: C#Cc1cnc(C2=CCN(c3nc4c(c(NC(C)(C)COC(=O)NC)n3)S(=O)CC4)CC2)nc1]

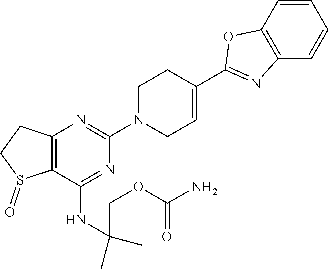 [SMILES: CC(C)(COC(N)=O)Nc1nc(N2CC=C(c3nc4ccccc4o3)CC2)nc2c1S(=O)CC2]

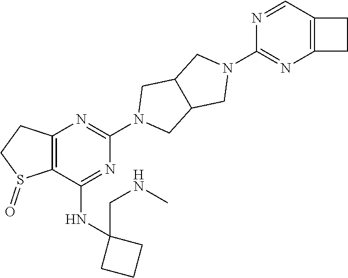 [SMILES: CNCC1(Nc2nc(N3CC4CN(c5ncc6c(n5)CC6)CC4C3)nc3c2S(=O)CC3)CCC1]

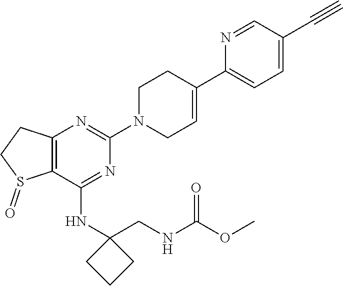 [SMILES: C#Cc1ccc(C2=CCN(c3nc4c(c(NC5(CNC(=O)OC)CCC5)n3)S(=O)CC4)CC2)nc1]

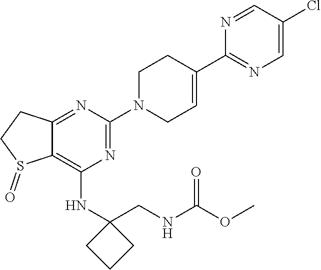 [SMILES: COC(=O)NCC1(Nc2nc(N3CC=C(c4ncc(Cl)cn4)CC3)nc3c2S(=O)CC3)CCC1]

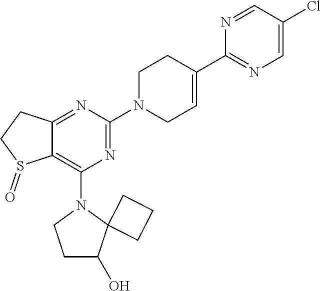 [SMILES: O=S1CCc2nc(N3CC=C(c4ncc(Cl)cn4)CC3)nc(N3CCC(O)C34CCC4)c21]

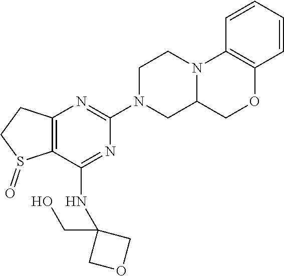 [SMILES: O=S1CCc2nc(N3CCN4c5ccccc5OCC4C3)nc(NC3(CO)COC3)c21]

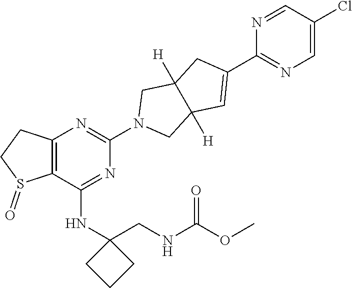 [SMILES: COC(=O)NCC1(Nc2nc(N3CC4C=C(c5ncc(Cl)cn5)CC4C3)nc3c2S(=O)CC3)CCC1]

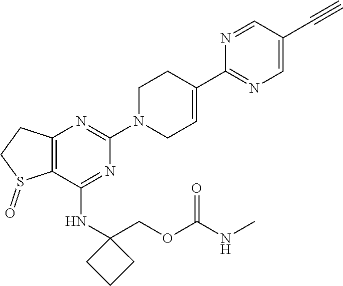 [SMILES: C#Cc1cnc(C2=CCN(c3nc4c(c(NC5(COC(=O)NC)CCC5)n3)S(=O)CC4)CC2)nc1]

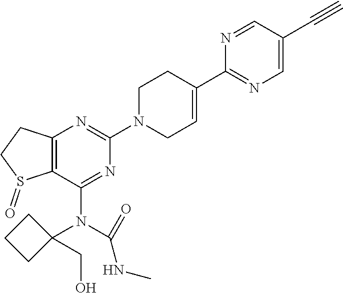 [SMILES: C#Cc1cnc(C2=CCN(c3nc4c(c(N(C(=O)NC)C5(CO)CCC5)n3)S(=O)CC4)CC2)nc1]

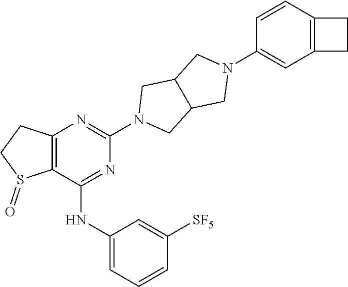

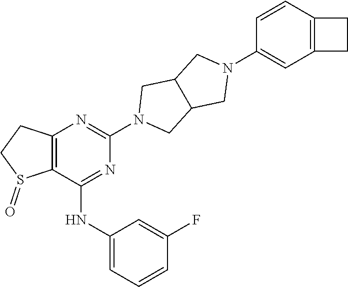 [SMILES: O=S1CCc2nc(N3CC4CN(c5ccc6c(c5)CC6)CC4C3)nc(Nc3cccc(F)c3)c21]

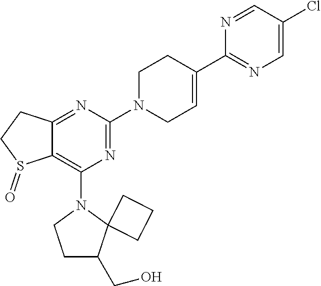 [SMILES: O=S1CCc2nc(N3CC=C(c4ncc(Cl)cn4)CC3)nc(N3CCC(CO)C34CCC4)c21]

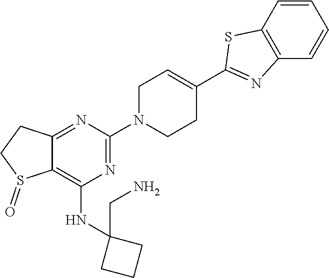 [SMILES: NCC1(Nc2nc(N3CC=C(c4nc5ccccc5s4)CC3)nc3c2S(=O)CC3)CCC1]

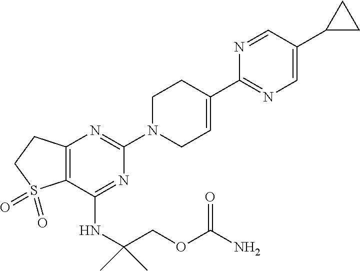 [SMILES: CC(C)(COC(N)=O)Nc1nc(N2CC=C(c3ncc(C4CC4)cn3)CC2)nc2c1S(=O)(=O)CC2]

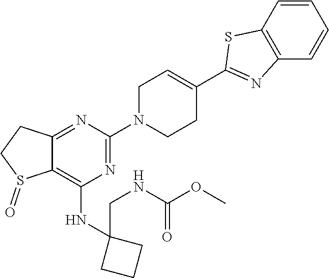 [SMILES: COC(=O)NCC1(Nc2nc(N3CC=C(c4nc5ccccc5s4)CC3)nc3c2S(=O)CC3)CCC1]

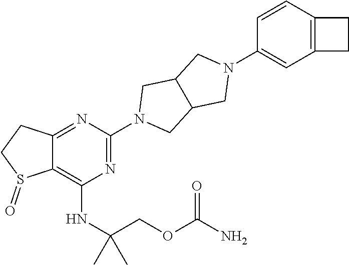 [SMILES: CC(C)(COC(N)=O)Nc1nc(N2CC3CN(c4ccc5c(c4)CC5)CC3C2)nc2c1S(=O)CC2]

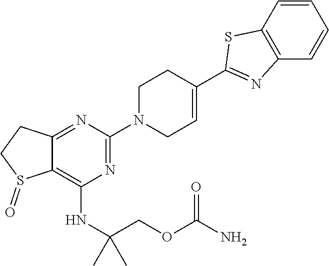 [SMILES: CC(C)(COC(N)=O)Nc1nc(N2CC=C(c3nc4ccccc4s3)CC2)nc2c1S(=O)CC2]

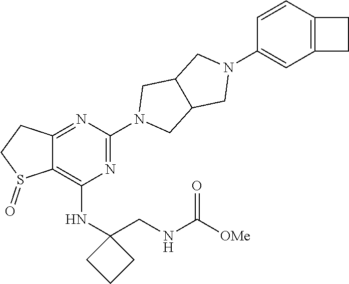 [SMILES: COC(=O)NCC1(Nc2nc(N3CC4CN(c5ccc6c(c5)CC6)CC4C3)nc3c2S(=O)CC3)CCC1]

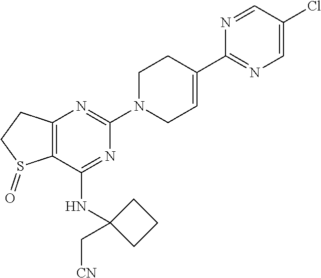 [SMILES: N#CCC1(Nc2nc(N3CC=C(c4ncc(Cl)cn4)CC3)nc3c2S(=O)CC3)CCC1]

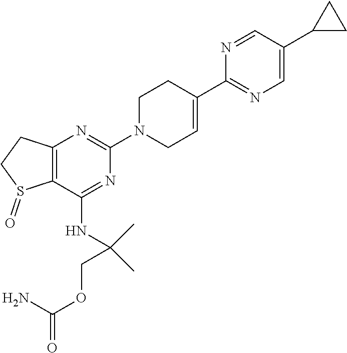 [SMILES: CC(C)(COC(N)=O)Nc1nc(N2CC=C(c3ncc(C4CC4)cn3)CC2)nc2c1S(=O)CC2]

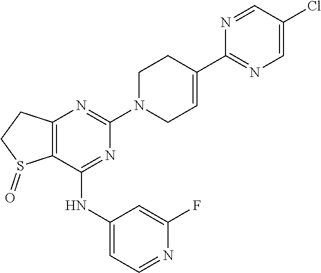 [SMILES: O=S1CCc2nc(N3CC=C(c4ncc(Cl)cn4)CC3)nc(Nc3ccnc(F)c3)c21]

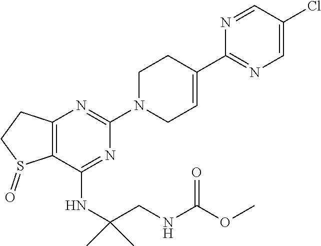 [SMILES: COC(=O)NCC(C)(C)Nc1nc(N2CC=C(c3ncc(Cl)cn3)CC2)nc2c1S(=O)CC2]

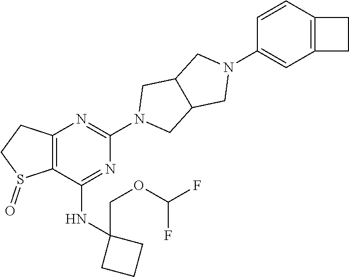 [SMILES: O=S1CCc2nc(N3CC4CN(c5ccc6c(c5)CC6)CC4C3)nc(NC3(COC(F)F)CCC3)c21]

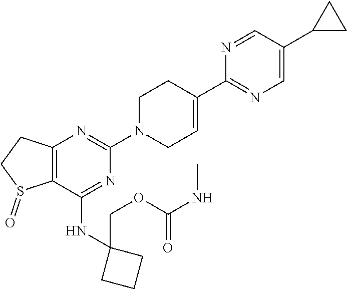 [SMILES: CNC(=O)OCC1(Nc2nc(N3CC=C(c4ncc(C5CC5)cn4)CC3)nc3c2S(=O)CC3)CCC1]

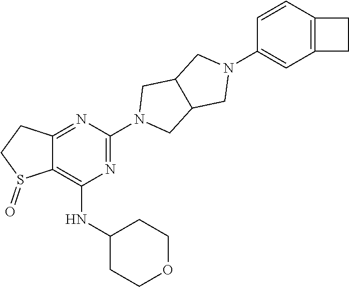 [SMILES: O=S1CCc2nc(N3CC4CN(c5ccc6c(c5)CC6)CC4C3)nc(NC3CCOCC3)c21]

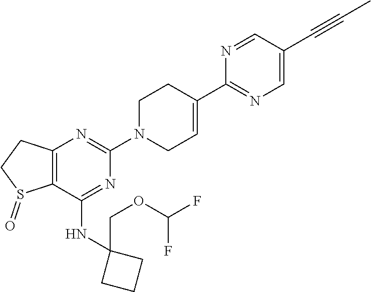 [SMILES: CC#Cc1cnc(C2=CCN(c3nc4c(c(NC5(COC(F)F)CCC5)n3)S(=O)CC4)CC2)nc1]

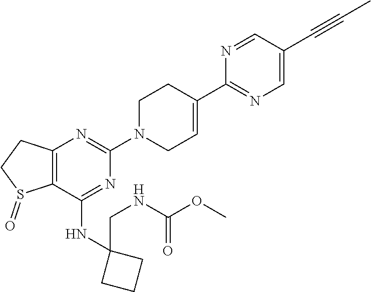 [SMILES: CC#Cc1cnc(C2=CCN(c3nc4c(c(NC5(CNC(=O)OC)CCC5)n3)S(=O)CC4)CC2)nc1]

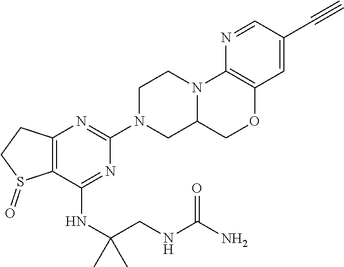 [SMILES: C#Cc1cnc2c(c1)OCC1CN(c3nc4c(c(NC(C)(C)CNC(N)=O)n3)S(=O)CC4)CCN21]

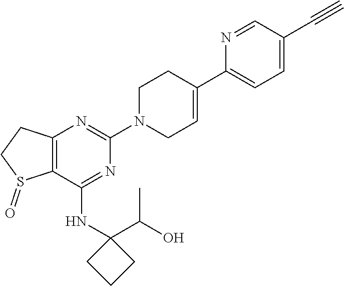 [SMILES: C#Cc1ccc(C2=CCN(c3nc4c(c(NC5(C(C)O)CCC5)n3)S(=O)CC4)CC2)nc1]

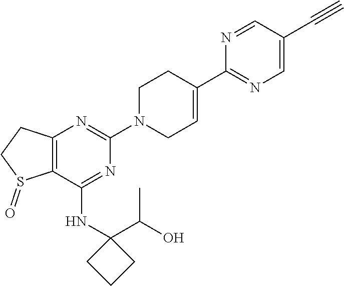 [SMILES: C#Cc1cnc(C2=CCN(c3nc4c(c(NC5(C(C)O)CCC5)n3)S(=O)CC4)CC2)nc1]

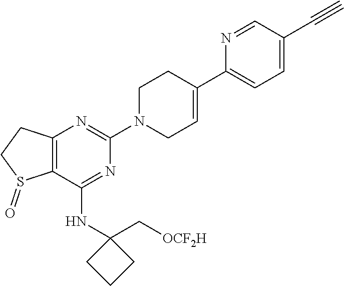

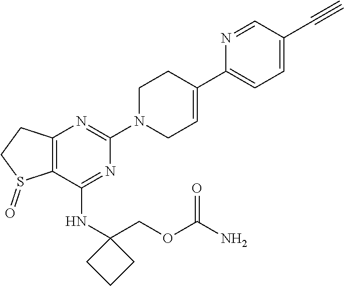 [SMILES: C#Cc1ccc(C2=CCN(c3nc4c(c(NC5(COC(N)=O)CCC5)n3)S(=O)CC4)CC2)nc1]

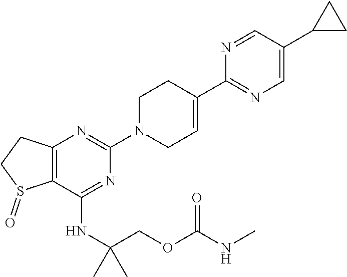 [SMILES: CNC(=O)OCC(C)(C)Nc1nc(N2CC=C(c3ncc(C4CC4)cn3)CC2)nc2c1S(=O)CC2]

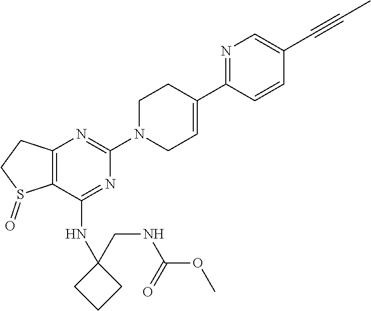 [SMILES: CC#Cc1ccc(C2=CCN(c3nc4c(c(NC5(CNC(=O)OC)CCC5)n3)S(=O)CC4)CC2)nc1]

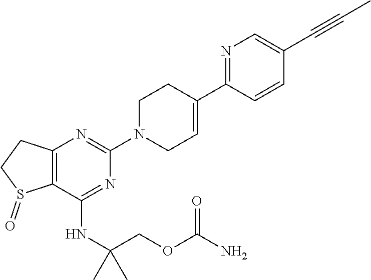 [SMILES: CC#Cc1ccc(C2=CCN(c3nc4c(c(NC(C)(C)COC(N)=O)n3)S(=O)CC4)CC2)nc1]

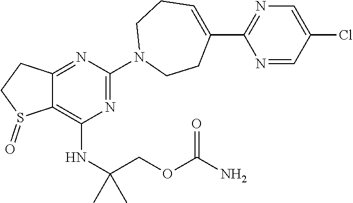 [SMILES: CC(C)(COC(N)=O)Nc1nc(N2CCC=C(c3ncc(Cl)cn3)CC2)nc2c1S(=O)CC2]

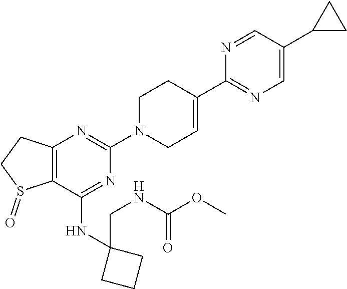 [SMILES: COC(=O)NCC1(Nc2nc(N3CC=C(c4ncc(C5CC5)cn4)CC3)nc3c2S(=O)CC3)CCC1]

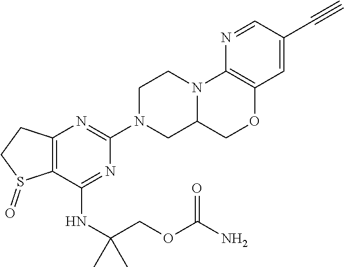 [SMILES: C#Cc1cnc2c(c1)OCC1CN(c3nc4c(c(NC(C)(C)COC(N)=O)n3)S(=O)CC4)CCN21]

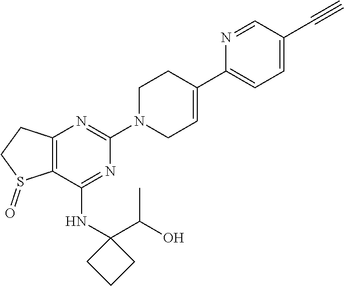 [SMILES: C#Cc1ccc(C2=CCN(c3nc4c(c(NC5(C(C)O)CCC5)n3)S(=O)CC4)CC2)nc1]

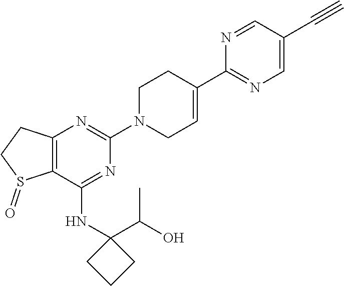 [SMILES: C#Cc1cnc(C2=CCN(c3nc4c(c(NC5(C(C)O)CCC5)n3)S(=O)CC4)CC2)nc1]

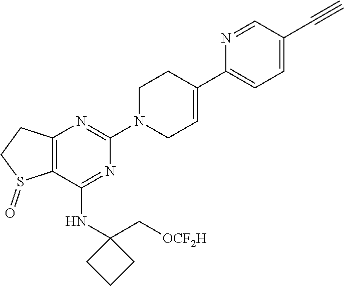

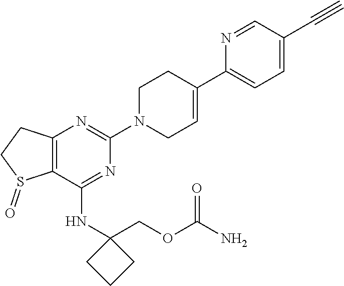 [SMILES: C#Cc1ccc(C2=CCN(c3nc4c(c(NC5(COC(N)=O)CCC5)n3)S(=O)CC4)CC2)nc1]

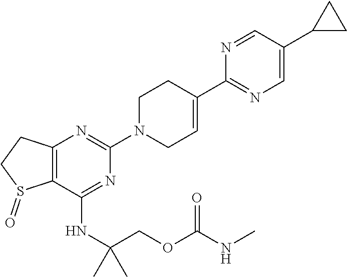 [SMILES: CNC(=O)OCC(C)(C)Nc1nc(N2CC=C(c3ncc(C4CC4)cn3)CC2)nc2c1S(=O)CC2]

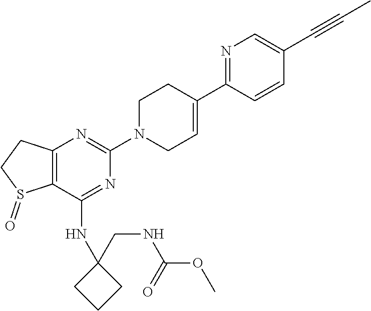 [SMILES: CC#Cc1ccc(C2=CCN(c3nc4c(c(NC5(CNC(=O)OC)CCC5)n3)S(=O)CC4)CC2)nc1]

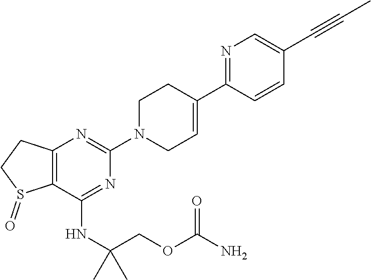 [SMILES: CC#Cc1ccc(C2=CCN(c3nc4c(c(NC(C)(C)COC(N)=O)n3)S(=O)CC4)CC2)nc1]

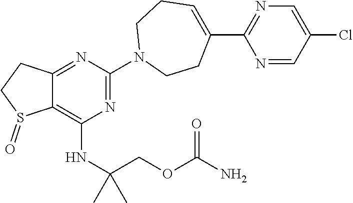 [SMILES: CC(C)(COC(N)=O)Nc1nc(N2CCC=C(c3ncc(Cl)cn3)CC2)nc2c1S(=O)CC2]

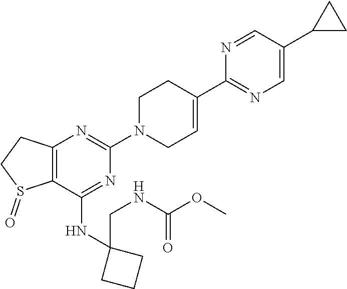 [SMILES: COC(=O)NCC1(Nc2nc(N3CC=C(c4ncc(C5CC5)cn4)CC3)nc3c2S(=O)CC3)CCC1]

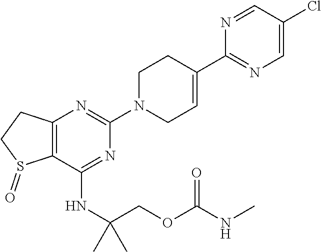 [SMILES: CNC(=O)OCC(C)(C)Nc1nc(N2CC=C(c3ncc(Cl)cn3)CC2)nc2c1S(=O)CC2]

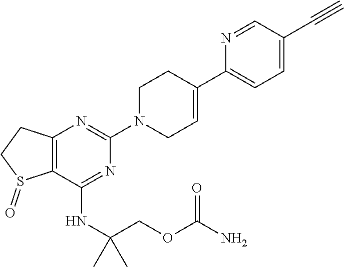 [SMILES: C#Cc1ccc(C2=CCN(c3nc4c(c(NC(C)(C)COC(N)=O)n3)S(=O)CC4)CC2)nc1]

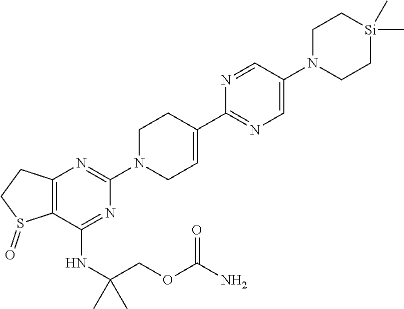 [SMILES: CC(C)(COC(N)=O)Nc1nc(N2CC=C(c3ncc(N4CC[Si](C)(C)CC4)cn3)CC2)nc2c1S(=O)CC2]

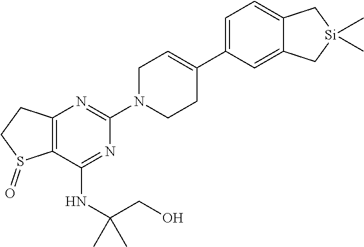 [SMILES: CC(C)(CO)Nc1nc(N2CC=C(c3ccc4c(c3)C[Si](C)(C)C4)CC2)nc2c1S(=O)CC2]

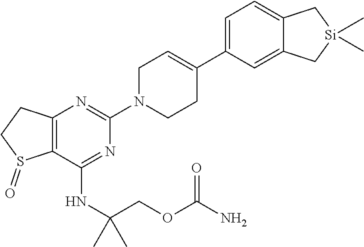 [SMILES: CC(C)(COC(N)=O)Nc1nc(N2CC=C(c3ccc4c(c3)C[Si](C)(C)C4)CC2)nc2c1S(=O)CC2]

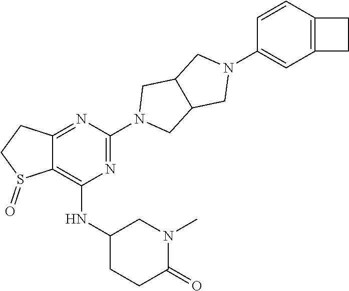 [SMILES: CN1CC(Nc2nc(N3CC4CN(c5ccc6c(c5)CC6)CC4C3)nc3c2S(=O)CC3)CCC1=O]

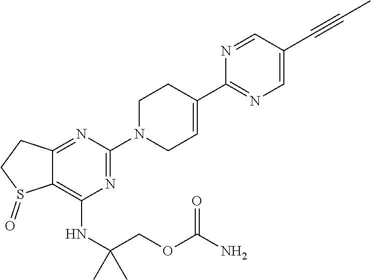 [SMILES: CC#Cc1cnc(C2=CCN(c3nc4c(c(NC(C)(C)COC(N)=O)n3)S(=O)CC4)CC2)nc1]

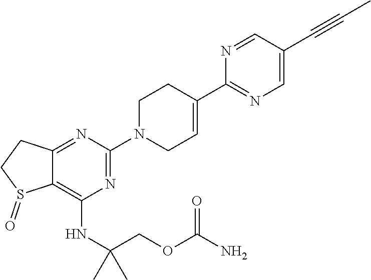 [SMILES: CC#Cc1cnc(C2=CCN(c3nc4c(c(NC(C)(C)COC(N)=O)n3)S(=O)CC4)CC2)nc1]

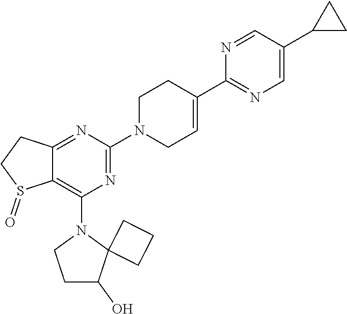 [SMILES: O=S1CCc2nc(N3CC=C(c4ncc(C5CC5)cn4)CC3)nc(N3CCC(O)C34CCC4)c21]

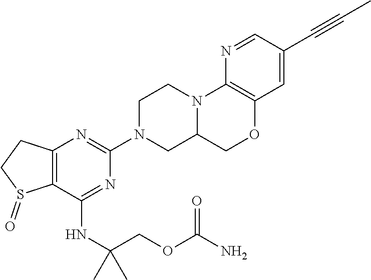 [SMILES: CC#Cc1cnc2c(c1)OCC1CN(c3nc4c(c(NC(C)(C)COC(N)=O)n3)S(=O)CC4)CCN21]

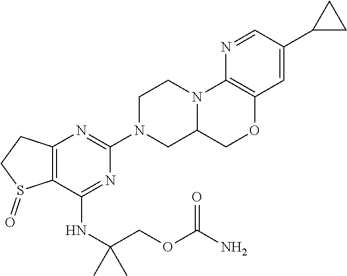 [SMILES: CC(C)(COC(N)=O)Nc1nc(N2CCN3c4ncc(C5CC5)cc4OCC3C2)nc2c1S(=O)CC2]

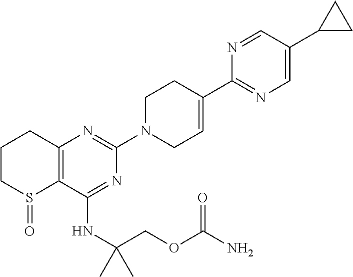 [SMILES: CC(C)(COC(N)=O)Nc1nc(N2CC=C(c3ncc(C4CC4)cn3)CC2)nc2c1S(=O)CCC2]

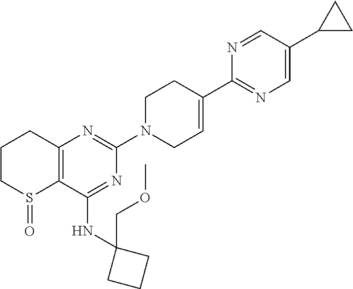 [SMILES: COCC1(Nc2nc(N3CC=C(c4ncc(C5CC5)cn4)CC3)nc3c2S(=O)CCC3)CCC1]

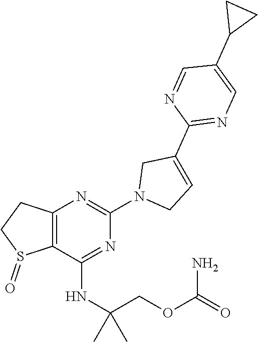 [SMILES: CC(C)(COC(N)=O)Nc1nc(N2CC=C(c3ncc(C4CC4)cn3)C2)nc2c1S(=O)CC2]

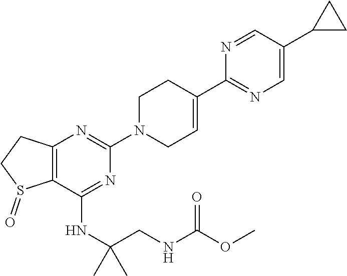 [SMILES: COC(=O)NCC(C)(C)Nc1nc(N2CC=C(c3ncc(C4CC4)cn3)CC2)nc2c1S(=O)CC2]

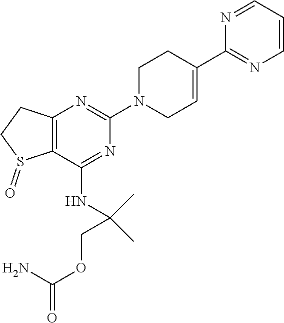 [SMILES: CC(C)(COC(N)=O)Nc1nc(N2CC=C(c3ncccn3)CC2)nc2c1S(=O)CC2]

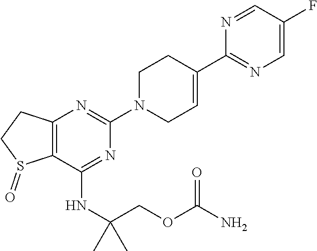 [SMILES: CC(C)(COC(N)=O)Nc1nc(N2CC=C(c3ncc(F)cn3)CC2)nc2c1S(=O)CC2]

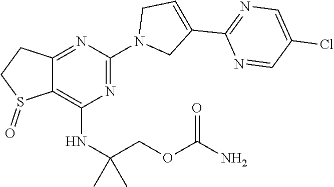 [SMILES: CC(C)(COC(N)=O)Nc1nc(N2CC=C(c3ncc(Cl)cn3)C2)nc2c1S(=O)CC2]

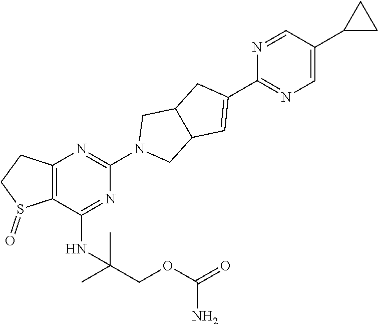 [SMILES: CC(C)(COC(N)=O)Nc1nc(N2CC3C=C(c4ncc(C5CC5)cn4)CC3C2)nc2c1S(=O)CC2]

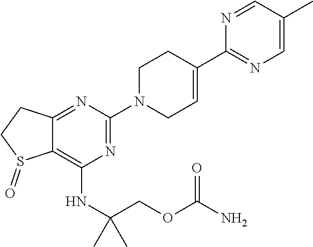 [SMILES: Cc1cnc(C2=CCN(c3nc4c(c(NC(C)(C)COC(N)=O)n3)S(=O)CC4)CC2)nc1]

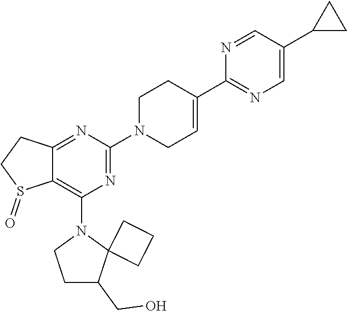 [SMILES: O=S1CCc2nc(N3CC=C(c4ncc(C5CC5)cn4)CC3)nc(N3CCC(CO)C34CCC4)c21]

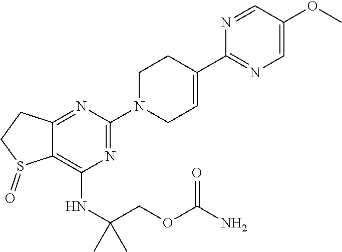 [SMILES: COc1cnc(C2=CCN(c3nc4c(c(NC(C)(C)COC(N)=O)n3)S(=O)CC4)CC2)nc1]

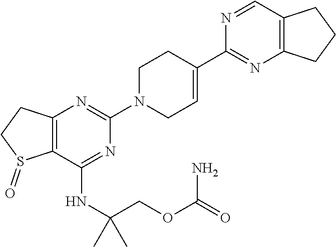 [SMILES: CC(C)(COC(N)=O)Nc1nc(N2CC=C(c3ncc4c(n3)CCC4)CC2)nc2c1S(=O)CC2]

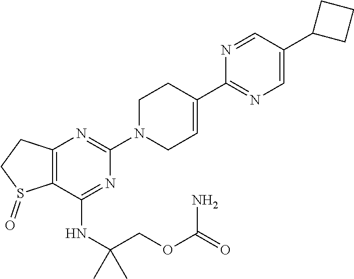 [SMILES: CC(C)(COC(N)=O)Nc1nc(N2CC=C(c3ncc(C4CCC4)cn3)CC2)nc2c1S(=O)CC2]

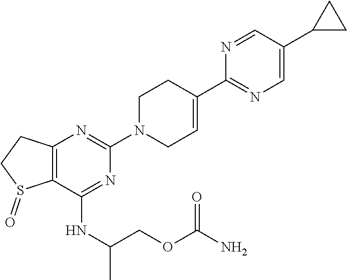 [SMILES: CC(COC(N)=O)Nc1nc(N2CC=C(c3ncc(C4CC4)cn3)CC2)nc2c1S(=O)CC2]

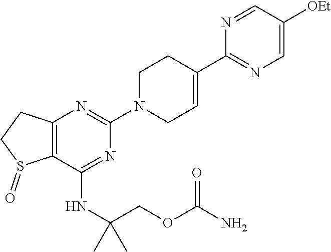 [SMILES: CCOc1cnc(C2=CCN(c3nc4c(c(NC(C)(C)COC(N)=O)n3)S(=O)CC4)CC2)nc1]

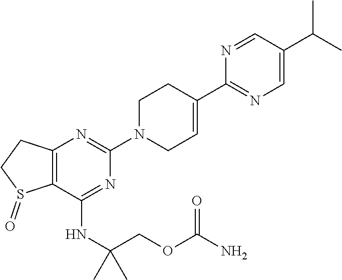 [SMILES: CC(C)c1cnc(C2=CCN(c3nc4c(c(NC(C)(C)COC(N)=O)n3)S(=O)CC4)CC2)nc1]

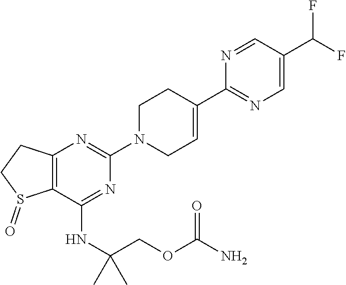 [SMILES: CC(C)(COC(N)=O)Nc1nc(N2CC=C(c3ncc(C(F)F)cn3)CC2)nc2c1S(=O)CC2]

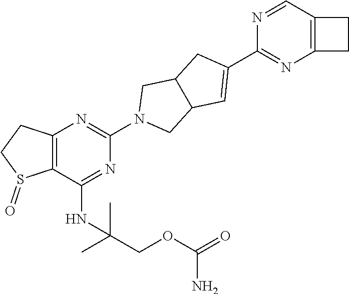 [SMILES: CC(C)(COC(N)=O)Nc1nc(N2CC3C=C(c4ncc5c(n4)CC5)CC3C2)nc2c1S(=O)CC2]

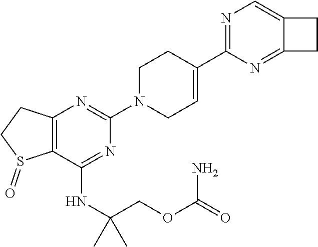 [SMILES: CC(C)(COC(N)=O)Nc1nc(N2CC=C(c3ncc4c(n3)CC4)CC2)nc2c1S(=O)CC2]

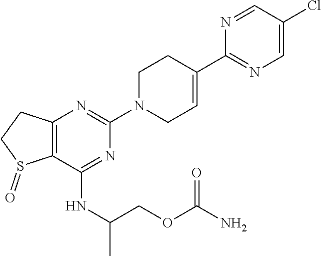 [SMILES: CC(COC(N)=O)Nc1nc(N2CC=C(c3ncc(Cl)cn3)CC2)nc2c1S(=O)CC2]

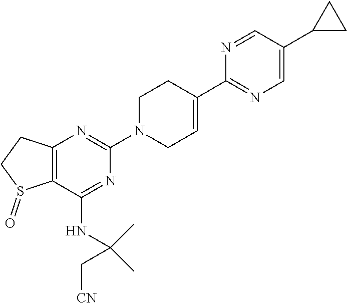 [SMILES: CC(C)(CC#N)Nc1nc(N2CC=C(c3ncc(C4CC4)cn3)CC2)nc2c1S(=O)CC2]

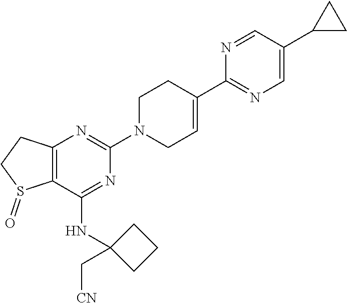 [SMILES: N#CCC1(Nc2nc(N3CC=C(c4ncc(C5CC5)cn4)CC3)nc3c2S(=O)CC3)CCC1]

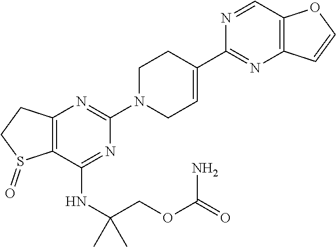 [SMILES: CC(C)(COC(N)=O)Nc1nc(N2CC=C(c3ncc4occc4n3)CC2)nc2c1S(=O)CC2]

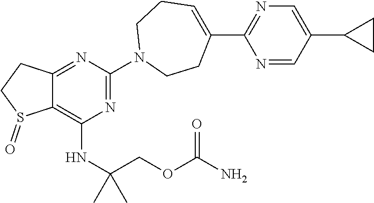 [SMILES: CC(C)(COC(N)=O)Nc1nc(N2CCC=C(c3ncc(C4CC4)cn3)CC2)nc2c1S(=O)CC2]

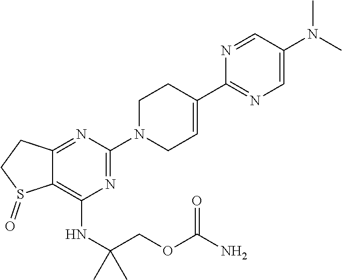 [SMILES: CN(C)c1cnc(C2=CCN(c3nc4c(c(NC(C)(C)COC(N)=O)n3)S(=O)CC4)CC2)nc1]

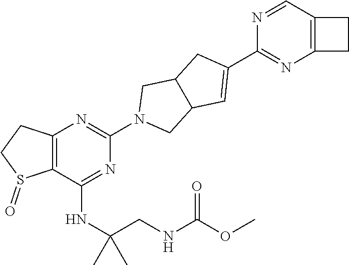 [SMILES: COC(=O)NCC(C)(C)Nc1nc(N2CC3C=C(c4ncc5c(n4)CC5)CC3C2)nc2c1S(=O)CC2]

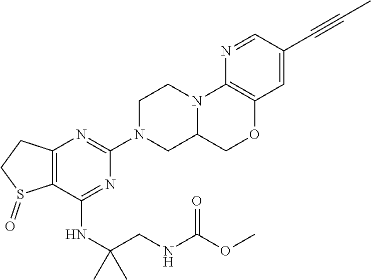 [SMILES: CC#Cc1cnc2c(c1)OCC1CN(c3nc4c(c(NC(C)(C)CNC(=O)OC)n3)S(=O)CC4)CCN21]

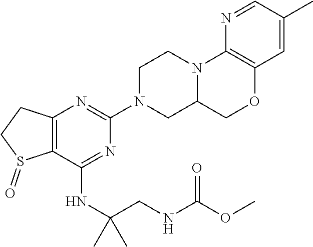 [SMILES: COC(=O)NCC(C)(C)Nc1nc(N2CCN3c4ncc(C)cc4OCC3C2)nc2c1S(=O)CC2]

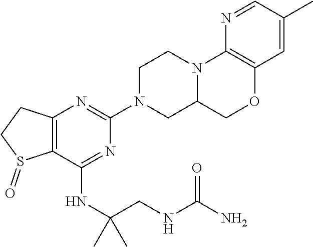 [SMILES: Cc1cnc2c(c1)OCC1CN(c3nc4c(c(NC(C)(C)CNC(N)=O)n3)S(=O)CC4)CCN21]

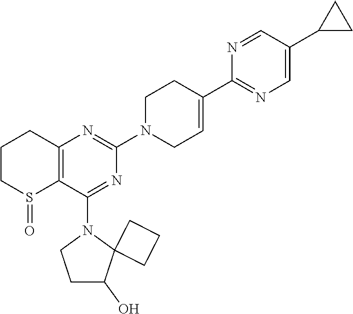 [SMILES: O=S1CCCc2nc(N3CC=C(c4ncc(C5CC5)cn4)CC3)nc(N3CCC(O)C34CCC4)c21]

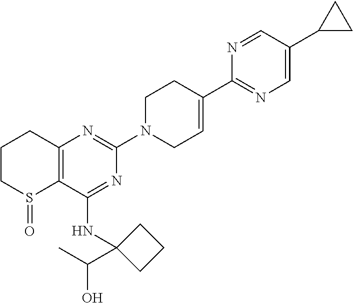 [SMILES: CC(O)C1(Nc2nc(N3CC=C(c4ncc(C5CC5)cn4)CC3)nc3c2S(=O)CCC3)CCC1]

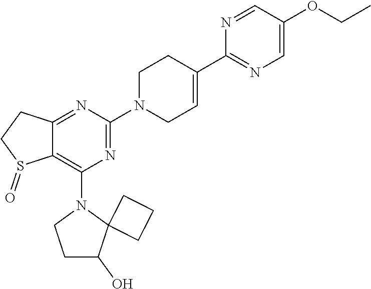 [SMILES: CCOc1cnc(C2=CCN(c3nc4c(c(N5CCC(O)C56CCC6)n3)S(=O)CC4)CC2)nc1]

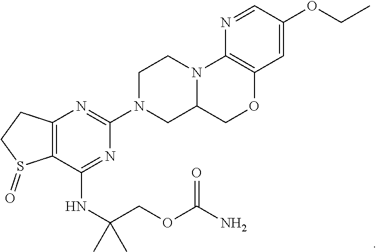 [SMILES: CCOc1cnc2c(c1)OCC1CN(c3nc4c(c(NC(C)(C)COC(N)=O)n3)S(=O)CC4)CCN21]

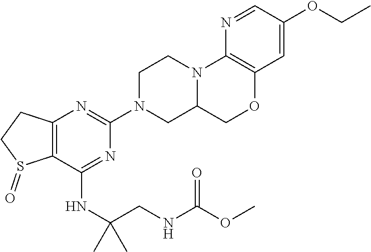 [SMILES: CCOc1cnc2c(c1)OCC1CN(c3nc4c(c(NC(C)(C)CNC(=O)OC)n3)S(=O)CC4)CCN21]

Solution 21 of the present disclosure relates to a compound selected from one of the structures in Table 2 below, or stereoisomers or pharmaceutically acceptable salts thereof:

TABLE 2

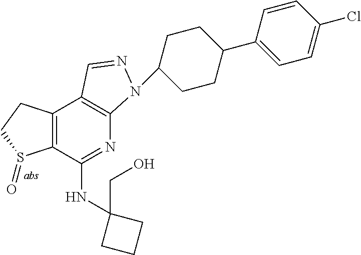 [SMILES: O=[S@@]1CCc2c1c(NC1(CO)CCC1)nc1c2cnn1C1CCC(c2ccc(Cl)cc2)CC1]

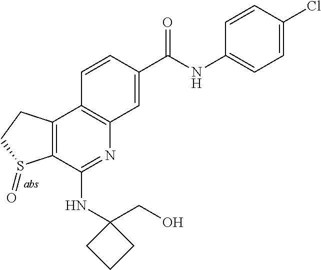 [SMILES: O=C(Nc1ccc(Cl)cc1)c1ccc2c3c(c(NC4(CO)CCC4)nc2c1)[S@](=O)CC3]

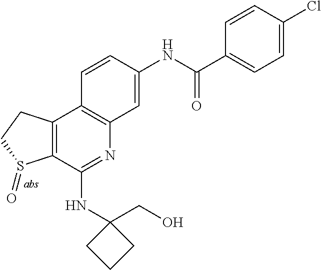 [SMILES: O=C(Nc1ccc2c3c(c(NC4(CO)CCC4)nc2c1)[S@](=O)CC3)c1ccc(Cl)cc1]

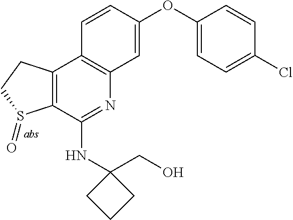 [SMILES: O=[S@@]1CCc2c1c(NC1(CO)CCC1)nc1cc(Oc3ccc(Cl)cc3)ccc21]

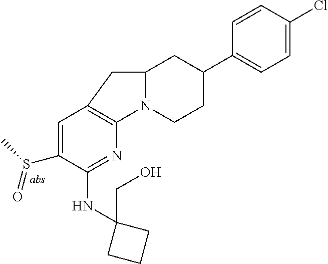 [SMILES: C[S@@](=O)c1cc2c(nc1NC1(CO)CCC1)N1CCC(c3ccc(Cl)cc3)CC1C2]

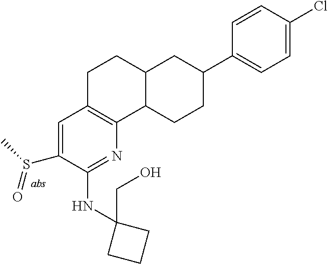 [SMILES: C[S@@](=O)c1cc2c(nc1NC1(CO)CCC1)C1CCC(c3ccc(Cl)cc3)CC1CC2]

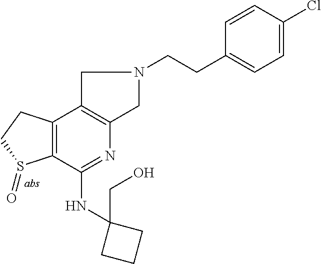 [SMILES: O=[S@@]1CCc2c3c(nc(NC4(CO)CCC4)c21)CN(CCc1ccc(Cl)cc1)C3]

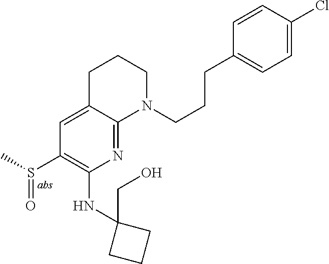 [SMILES: C[S@@](=O)c1cc2c(nc1NC1(CO)CCC1)N(CCCc1ccc(Cl)cc1)CCC2]

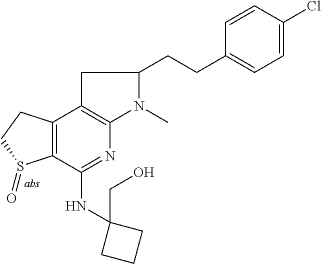 [SMILES: CN1c2nc(NC3(CO)CCC3)c3c(c2CC1CCc1ccc(Cl)cc1)CC[S@]3=O]

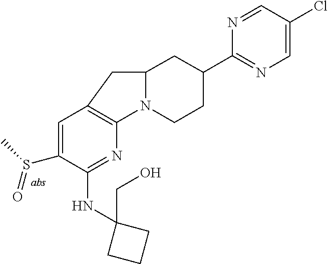 [SMILES: C[S@@](=O)c1cc2c(nc1NC1(CO)CCC1)N1CCC(c3ncc(Cl)cn3)CC1C2]

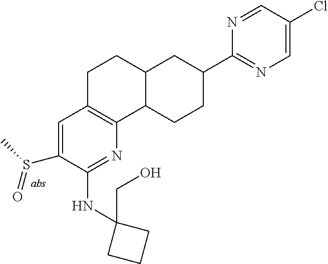 [SMILES: C[S@@](=O)c1cc2c(nc1NC1(CO)CCC1)C1CCC(c3ncc(Cl)cn3)CC1CC2]

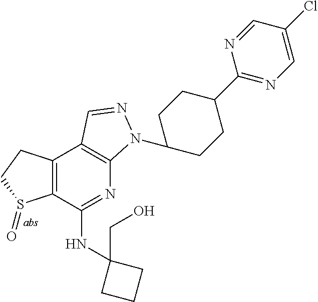 [SMILES: O=[S@@]1CCc2c1c(NC1(CO)CCC1)nc1c2cnn1C1CCC(c2ncc(Cl)cn2)CC1]

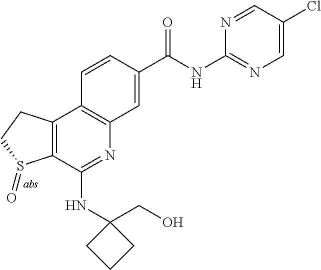 [SMILES: O=C(Nc1ncc(Cl)cn1)c1ccc2c3c(c(NC4(CO)CCC4)nc2c1)[S@](=O)CC3]

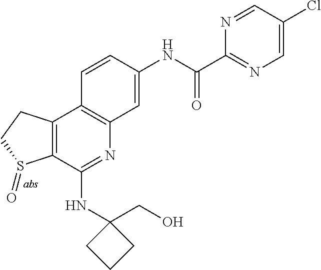 [SMILES: O=C(Nc1ccc2c3c(c(NC4(CO)CCC4)nc2c1)[S@](=O)CC3)c1ncc(Cl)cn1]

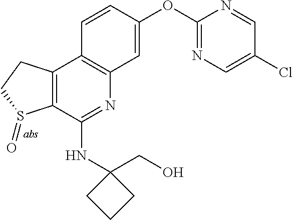 [SMILES: O=[S@@]1CCc2c1c(NC1(CO)CCC1)nc1cc(Oc3ncc(Cl)cn3)ccc21]

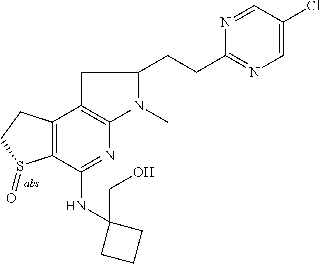 [SMILES: CN1c2nc(NC3(CO)CCC3)c3c(c2CC1CCc1ncc(Cl)cn1)CC[S@]3=O]

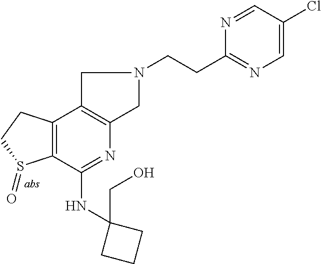 [SMILES: O=[S@@]1CCc2c3c(nc(NC4(CO)CCC4)c21)CN(CCc1ncc(Cl)cn1)C3]

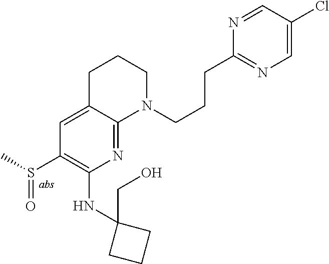 [SMILES: C[S@@](=O)c1cc2c(nc1NC1(CO)CCC1)N(CCCc1ncc(Cl)cn1)CCC2]

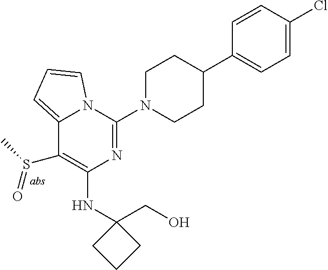 [SMILES: C[S@@](=O)c1c(NC2(CO)CCC2)nc(N2CCC(c3ccc(Cl)cc3)CC2)n2cccc12]

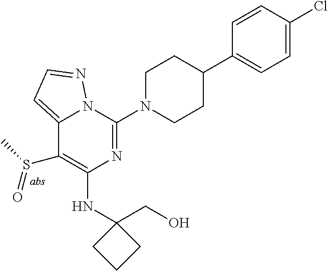 [SMILES: C[S@@](=O)c1c(NC2(CO)CCC2)nc(N2CCC(c3ccc(Cl)cc3)CC2)n2nccc12]

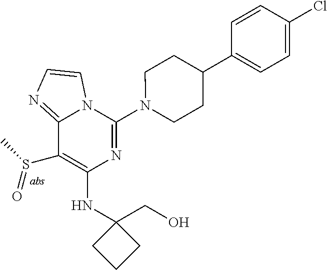 [SMILES: C[S@@](=O)c1c(NC2(CO)CCC2)nc(N2CCC(c3ccc(Cl)cc3)CC2)n2ccnc12]

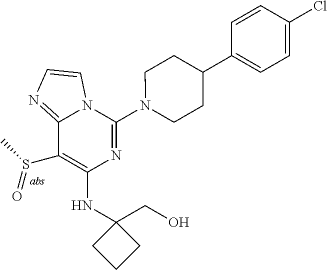 [SMILES: C[S@@](=O)c1c(NC2(CO)CCC2)nc(N2CCC(c3ccc(Cl)cc3)CC2)n2ccnc12]

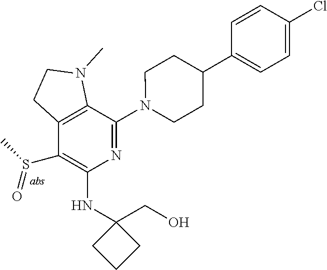 [SMILES: CN1CCc2c1c(N1CCC(c3ccc(Cl)cc3)CC1)nc(NC1(CO)CCC1)c2[S@@](C)=O]

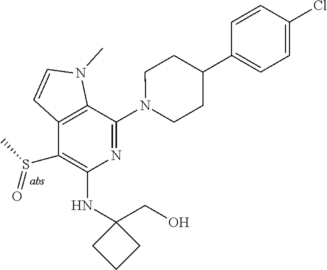 [SMILES: Cn1ccc2c([S@@](C)=O)c(NC3(CO)CCC3)nc(N3CCC(c4ccc(Cl)cc4)CC3)c21]

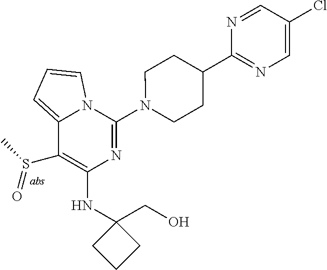 [SMILES: C[S@@](=O)c1c(NC2(CO)CCC2)nc(N2CCC(c3ncc(Cl)cn3)CC2)n2cccc12]

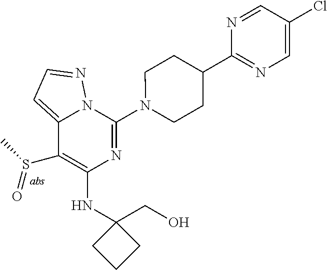 [SMILES: C[S@@](=O)c1c(NC2(CO)CCC2)nc(N2CCC(c3ncc(Cl)cn3)CC2)n2nccc12]

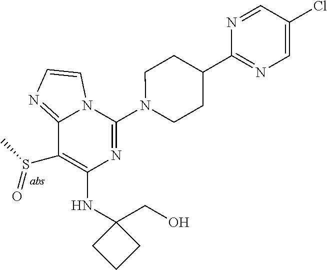 [SMILES: C[S@@](=O)c1c(NC2(CO)CCC2)nc(N2CCC(c3ncc(Cl)cn3)CC2)n2ccnc12]

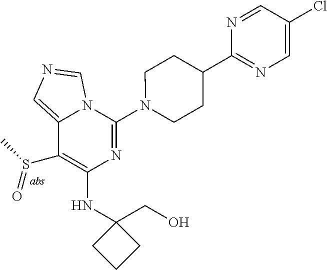 [SMILES: C[S@@](=O)c1c(NC2(CO)CCC2)nc(N2CCC(c3ncc(Cl)cn3)CC2)n2cncc12]

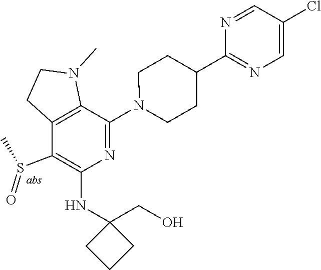 [SMILES: CN1CCc2c1c(N1CCC(c3ncc(Cl)cn3)CC1)nc(NC1(CO)CCC1)c2[S@@](C)=O]

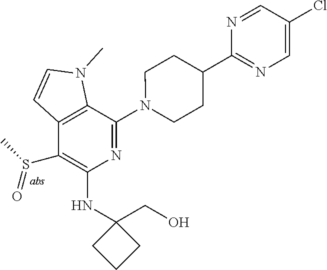 [SMILES: Cn1ccc2c([S@@](C)=O)c(NC3(CO)CCC3)nc(N3CCC(c4ncc(Cl)cn4)CC3)c21]

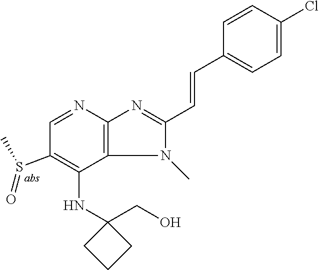 [SMILES: Cn1c(/C=C/c2ccc(Cl)cc2)nc2ncc([S@@](C)=O)c(NC3(CO)CCC3)c21]

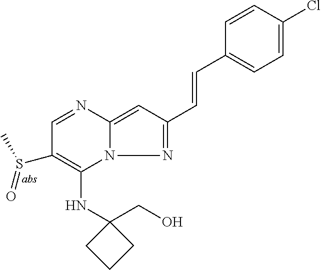 [SMILES: C[S@@](=O)c1cnc2cc(/C=C/c3ccc(Cl)cc3)nn2c1NC1(CO)CCC1]

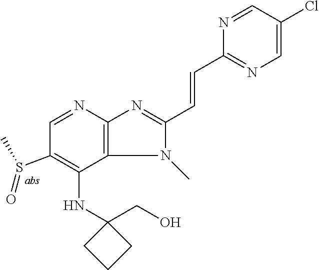 [SMILES: Cn1c(/C=C/c2ncc(Cl)cn2)nc2ncc([S@@](C)=O)c(NC3(CO)CCC3)c21]

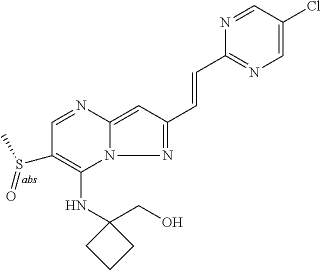 [SMILES: C[S@@](=O)c1cnc2cc(/C=C/c3ncc(Cl)cn3)nn2c1NC1(CO)CCC1]

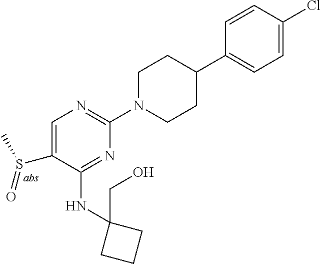 [SMILES: C[S@@](=O)c1cnc(N2CCC(c3ccc(Cl)cc3)CC2)nc1NC1(CO)CCC1]

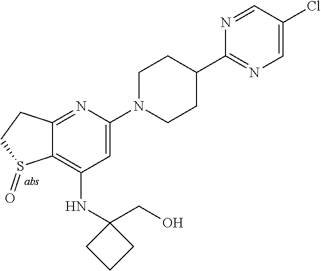 [SMILES: O=[S@@]1CCc2nc(N3CCC(c4ncc(Cl)cn4)CC3)cc(NC3(CO)CCC3)c21]

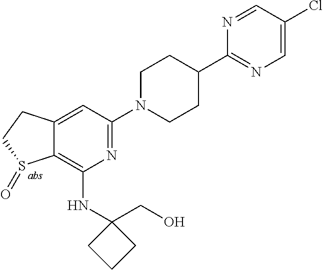 [SMILES: O=[S@@]1CCc2cc(N3CCC(c4ncc(Cl)cn4)CC3)nc(NC3(CO)CCC3)c21]

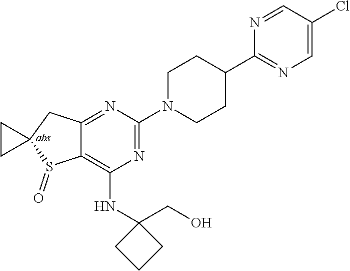

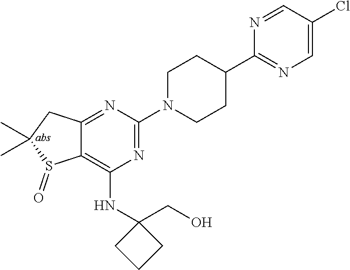

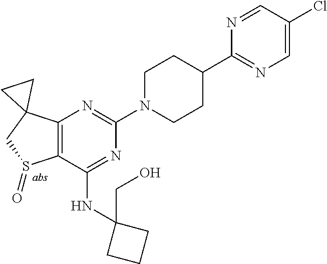 [SMILES: O=[S@@]1CC2(CC2)c2nc(N3CCC(c4ncc(Cl)cn4)CC3)nc(NC3(CO)CCC3)c21]

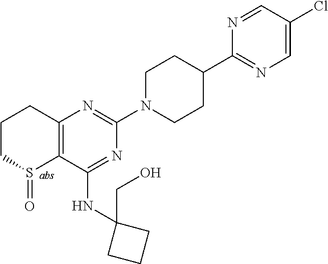 [SMILES: O=[S@@]1CCCc2nc(N3CCC(c4ncc(Cl)cn4)CC3)nc(NC3(CO)CCC3)c21]

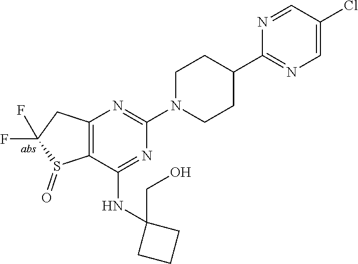

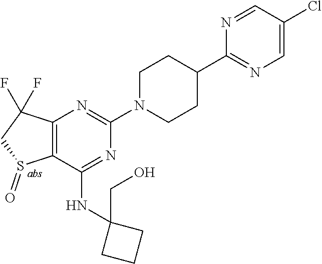 [SMILES: O=[S@@]1CC(F)(F)c2nc(N3CCC(c4ncc(Cl)cn4)CC3)nc(NC3(CO)CCC3)c21]

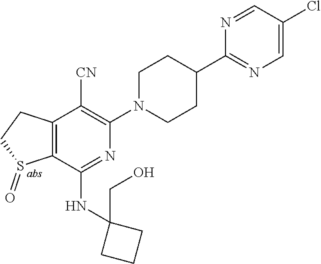 [SMILES: N#Cc1c(N2CCC(c3ncc(Cl)cn3)CC2)nc(NC2(CO)CCC2)c2c1CC[S@]2=O]

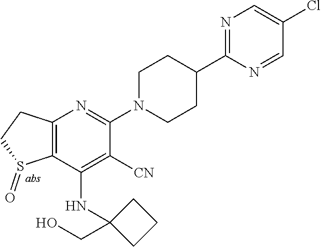 [SMILES: N#Cc1c(N2CCC(c3ncc(Cl)cn3)CC2)nc2c(c1NC1(CO)CCC1)[S@](=O)CC2]

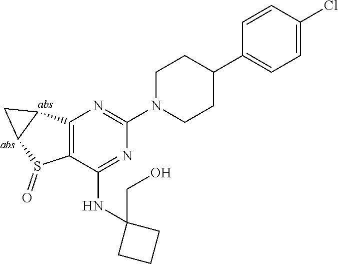

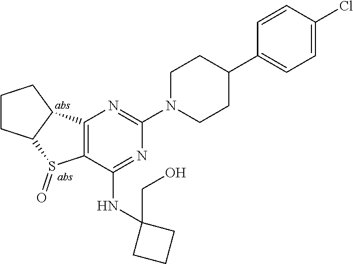

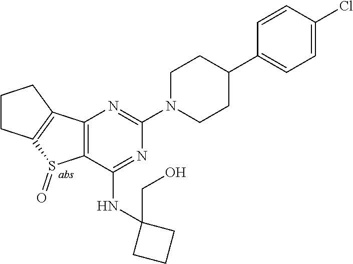 [SMILES: O=[S@@]1C2=C(CCC2)c2nc(N3CCC(c4ccc(Cl)cc4)CC3)nc(NC3(CO)CCC3)c21]

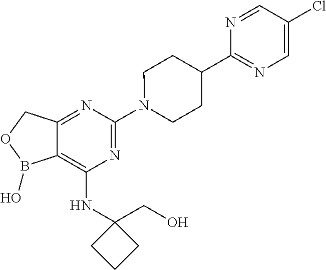 [SMILES: OCC1(Nc2nc(N3CCC(c4ncc(Cl)cn4)CC3)nc3c2B(O)OC3)CCC1]

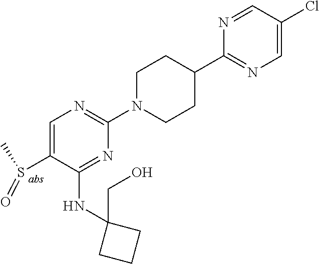 [SMILES: C[S@@](=O)c1cnc(N2CCC(c3ncc(Cl)cn3)CC2)nc1NC1(CO)CCC1]

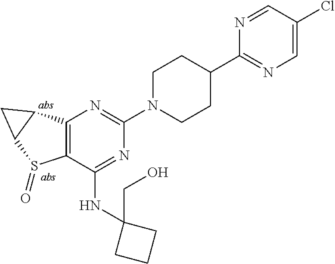

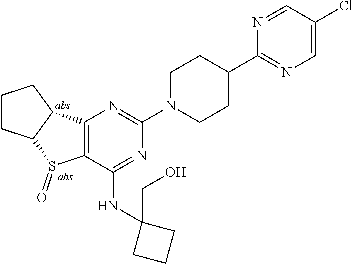

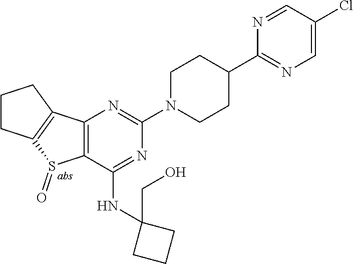 [SMILES: O=[S@@]1C2=C(CCC2)c2nc(N3CCC(c4ncc(Cl)cn4)CC3)nc(NC3(CO)CCC3)c21]

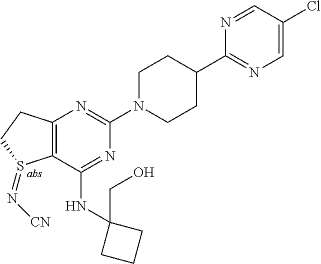 [SMILES: N#C/N=[S@@]1/CCc2nc(N3CCC(c4ncc(Cl)cn4)CC3)nc(NC3(CO)CCC3)c21]

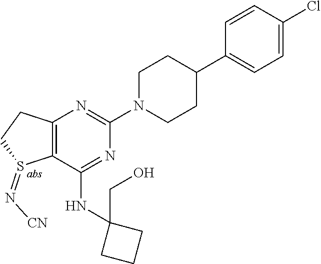 [SMILES: N#C/N=[S@@]1/CCc2nc(N3CCC(c4ccc(Cl)cc4)CC3)nc(NC3(CO)CCC3)c21]

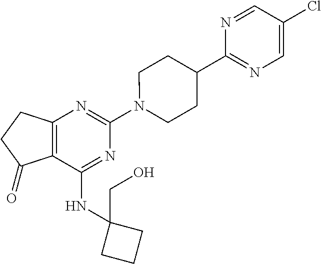 [SMILES: O=C1CCc2nc(N3CCC(c4ncc(Cl)cn4)CC3)nc(NC3(CO)CCC3)c21]

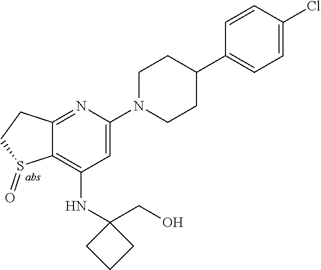 [SMILES: O=[S@@]1CCc2nc(N3CCC(c4ccc(Cl)cc4)CC3)cc(NC3(CO)CCC3)c21]

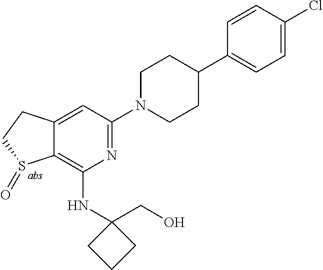 [SMILES: O=[S@@]1CCc2cc(N3CCC(c4ccc(Cl)cc4)CC3)nc(NC3(CO)CCC3)c21]

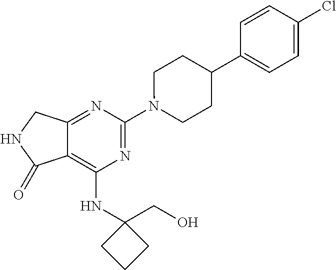 [SMILES: O=C1NCc2nc(N3CCC(c4ccc(Cl)cc4)CC3)nc(NC3(CO)CCC3)c21]

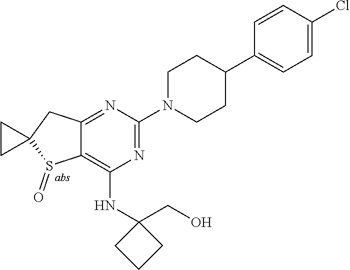 [SMILES: O=[S@]1c2c(nc(N3CCC(c4ccc(Cl)cc4)CC3)nc2NC2(CO)CCC2)CC12CC2]

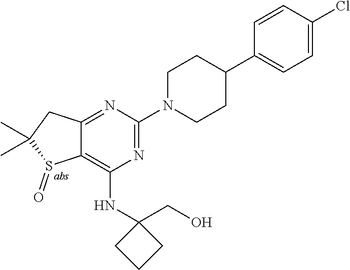 [SMILES: CC1(C)Cc2nc(N3CCC(c4ccc(Cl)cc4)CC3)nc(NC3(CO)CCC3)c2[S@@]1=O]

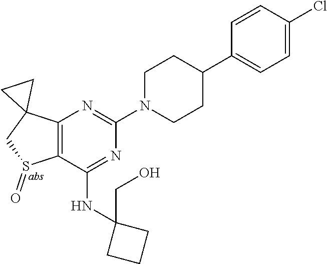 [SMILES: O=[S@@]1CC2(CC2)c2nc(N3CCC(c4ccc(Cl)cc4)CC3)nc(NC3(CO)CCC3)c21]

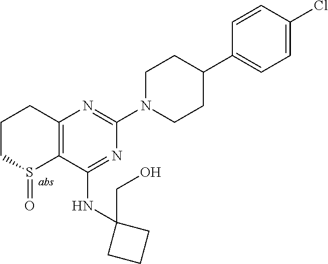 [SMILES: O=[S@@]1CCCc2nc(N3CCC(c4ccc(Cl)cc4)CC3)nc(NC3(CO)CCC3)c21]

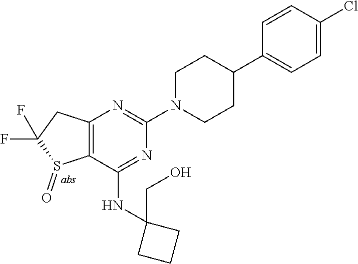 [SMILES: O=[S@]1c2c(nc(N3CCC(c4ccc(Cl)cc4)CC3)nc2NC2(CO)CCC2)CC1(F)F]

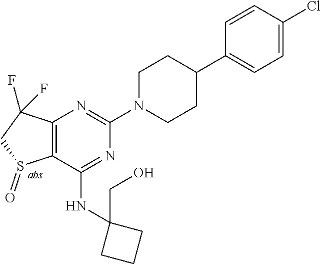 [SMILES: O=[S@@]1CC(F)(F)c2nc(N3CCC(c4ccc(Cl)cc4)CC3)nc(NC3(CO)CCC3)c21]

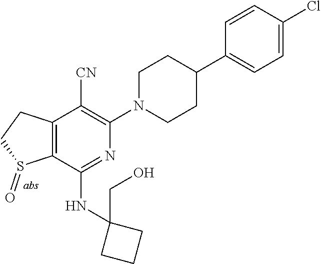 [SMILES: N#Cc1c(N2CCC(c3ccc(Cl)cc3)CC2)nc(NC2(CO)CCC2)c2c1CC[S@]2=O]

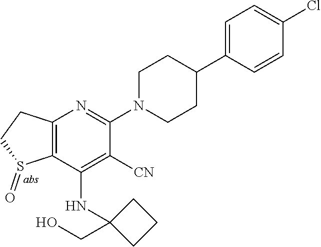 [SMILES: N#Cc1c(N2CCC(c3ccc(Cl)cc3)CC2)nc2c(c1NC1(CO)CCC1)[S@](=O)CC2]

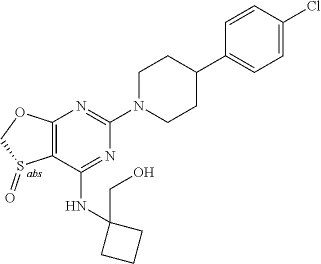 [SMILES: O=[S@@]1COc2nc(N3CCC(c4ccc(Cl)cc4)CC3)nc(NC3(CO)CCC3)c21]

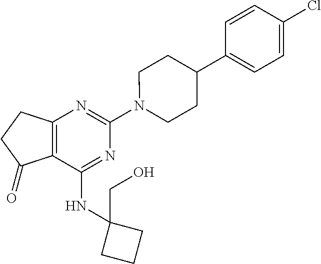 [SMILES: O=C1CCc2nc(N3CCC(c4ccc(Cl)cc4)CC3)nc(NC3(CO)CCC3)c21]

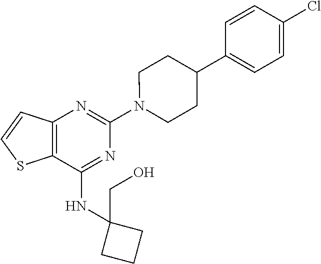 [SMILES: OCC1(Nc2nc(N3CCC(c4ccc(Cl)cc4)CC3)nc3ccsc23)CCC1]

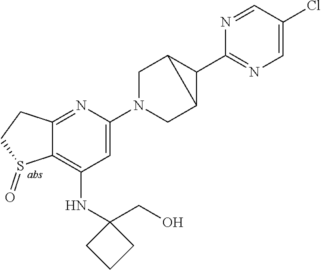 [SMILES: O=[S@@]1CCc2nc(N3CC4C(C3)C4c3ncc(Cl)cn3)cc(NC3(CO)CCC3)c21]

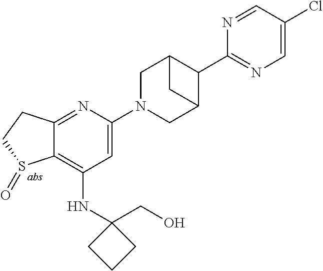 [SMILES: O=[S@@]1CCc2nc(N3CC4CC(C3)C4c3ncc(Cl)cn3)cc(NC3(CO)CCC3)c21]

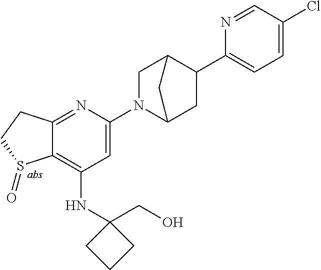 [SMILES: O=[S@@]1CCc2nc(N3CC4CC3CC4c3ccc(Cl)cn3)cc(NC3(CO)CCC3)c21]

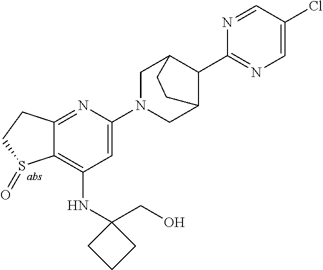 [SMILES: O=[S@@]1CCc2nc(N3CC4CCC(C3)C4c3ncc(Cl)cn3)cc(NC3(CO)CCC3)c21]

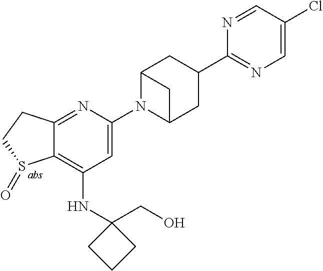 [SMILES: O=[S@@]1CCc2nc(N3C4CC(c5ncc(Cl)cn5)CC3C4)cc(NC3(CO)CCC3)c21]

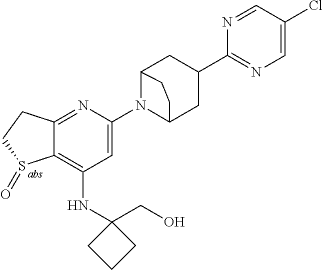 [SMILES: O=[S@@]1CCc2nc(N3C4CCC3CC(c3ncc(Cl)cn3)C4)cc(NC3(CO)CCC3)c21]

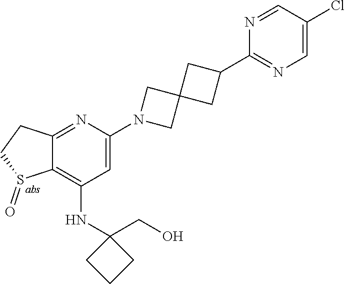 [SMILES: O=[S@@]1CCc2nc(N3CC4(CC(c5ncc(Cl)cn5)C4)C3)cc(NC3(CO)CCC3)c21]

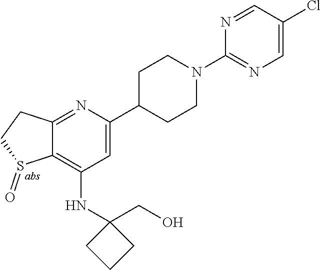 [SMILES: O=[S@@]1CCc2nc(C3CCN(c4ncc(Cl)cn4)CC3)cc(NC3(CO)CCC3)c21]

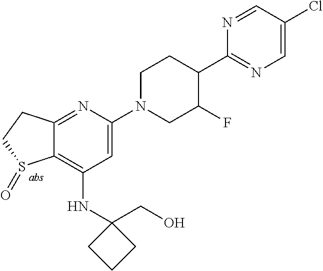 [SMILES: O=[S@@]1CCc2nc(N3CCC(c4ncc(Cl)cn4)C(F)C3)cc(NC3(CO)CCC3)c21]

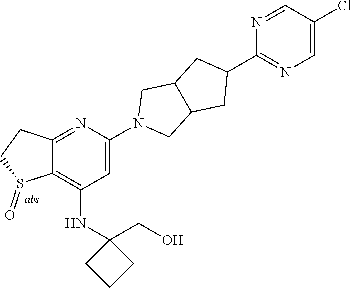 [SMILES: O=[S@@]1CCc2nc(N3CC4CC(c5ncc(Cl)cn5)CC4C3)cc(NC3(CO)CCC3)c21]

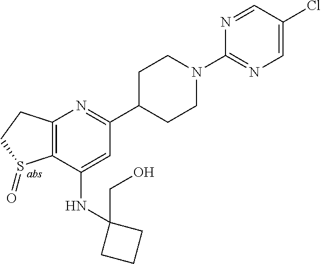 [SMILES: O=[S@@]1CCc2nc(C3CCN(c4ncc(Cl)cn4)CC3)cc(NC3(CO)CCC3)c21]

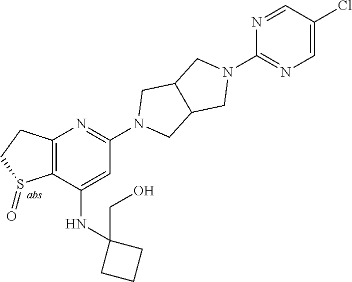 [SMILES: O=[S@@]1CCc2nc(N3CC4CN(c5ncc(Cl)cn5)CC4C3)cc(NC3(CO)CCC3)c21]

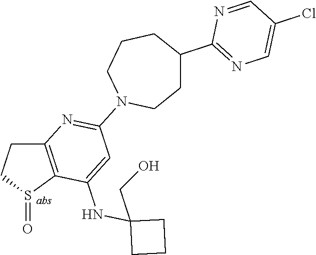 [SMILES: O=[S@@]1CCc2nc(N3CCCC(c4ncc(Cl)cn4)CC3)cc(NC3(CO)CCC3)c21]

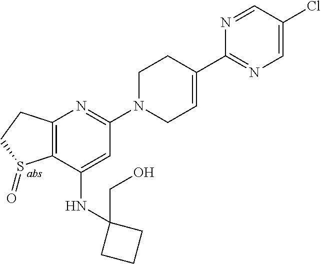 [SMILES: O=[S@@]1CCc2nc(N3CC=C(c4ncc(Cl)cn4)CC3)cc(NC3(CO)CCC3)c21]

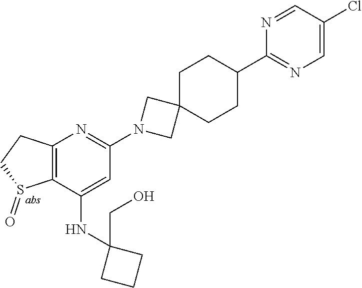 [SMILES: O=[S@@]1CCc2nc(N3CC4(CCC(c5ncc(Cl)cn5)CC4)C3)cc(NC3(CO)CCC3)c21]

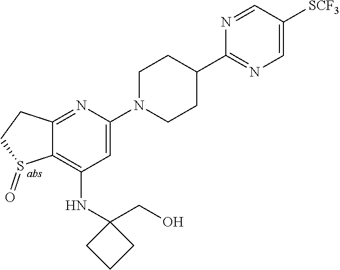

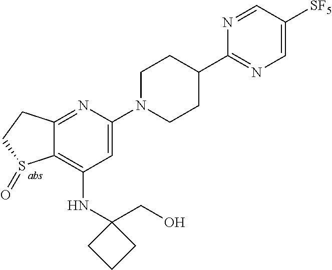

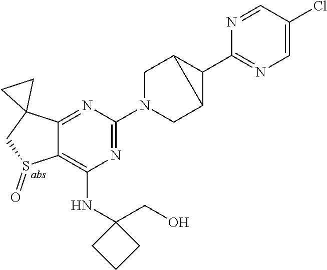 [SMILES: O=[S@@]1CC2(CC2)c2nc(N3CC4C(C3)C4c3ncc(Cl)cn3)nc(NC3(CO)CCC3)c21]

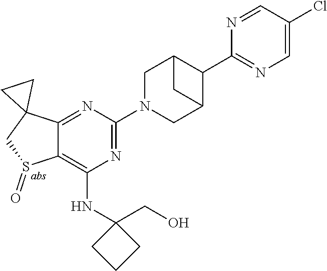 [SMILES: O=[S@@]1CC2(CC2)c2nc(N3CC4CC(C3)C4c3ncc(Cl)cn3)nc(NC3(CO)CCC3)c21]

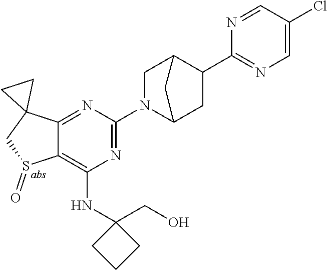 [SMILES: O=[S@@]1CC2(CC2)c2nc(N3CC4CC3CC4c3ncc(Cl)cn3)nc(NC3(CO)CCC3)c21]

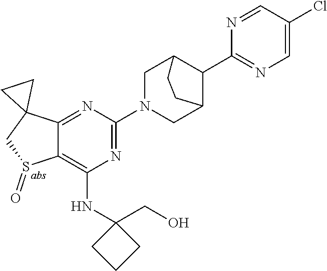 [SMILES: O=[S@@]1CC2(CC2)c2nc(N3CC4CCC(C3)C4c3ncc(Cl)cn3)nc(NC3(CO)CCC3)c21]

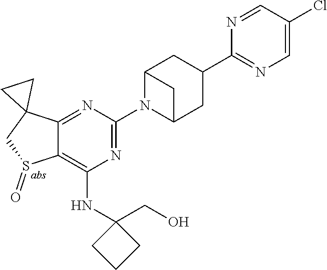 [SMILES: O=[S@@]1CC2(CC2)c2nc(N3C4CC(c5ncc(Cl)cn5)CC3C4)nc(NC3(CO)CCC3)c21]

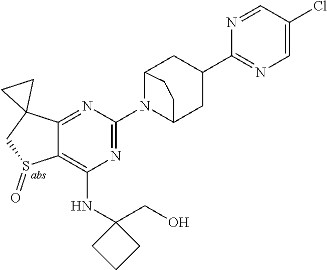 [SMILES: O=[S@@]1CC2(CC2)c2nc(N3C4CCC3CC(c3ncc(Cl)cn3)C4)nc(NC3(CO)CCC3)c21]

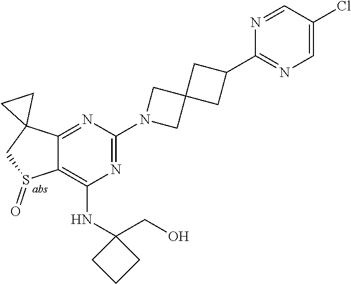 [SMILES: O=[S@@]1CC2(CC2)c2nc(N3CC4(CC(c5ncc(Cl)cn5)C4)C3)nc(NC3(CO)CCC3)c21]

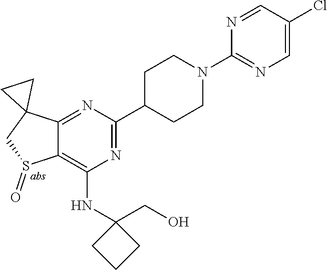 [SMILES: O=[S@@]1CC2(CC2)c2nc(C3CCN(c4ncc(Cl)cn4)CC3)nc(NC3(CO)CCC3)c21]

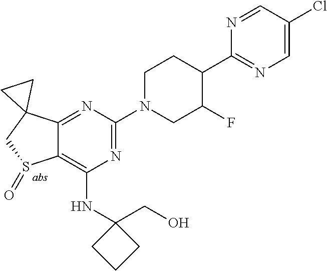 [SMILES: O=[S@@]1CC2(CC2)c2nc(N3CCC(c4ncc(Cl)cn4)C(F)C3)nc(NC3(CO)CCC3)c21]

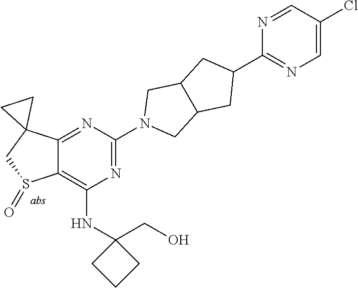 [SMILES: O=[S@@]1CC2(CC2)c2nc(N3CC4CC(c5ncc(Cl)cn5)CC4C3)nc(NC3(CO)CCC3)c21]

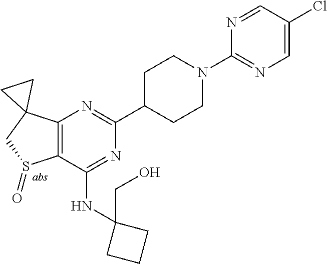 [SMILES: O=[S@@]1CC2(CC2)c2nc(C3CCN(c4ncc(Cl)cn4)CC3)nc(NC3(CO)CCC3)c21]

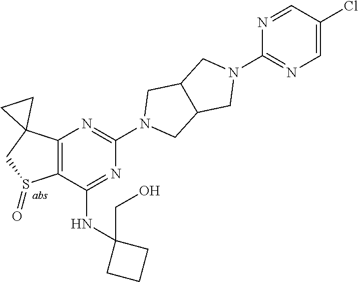 [SMILES: O=[S@@]1CC2(CC2)c2nc(N3CC4CN(c5ncc(Cl)cn5)CC4C3)nc(NC3(CO)CCC3)c21]

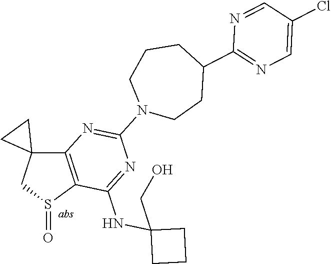 [SMILES: O=[S@@]1CC2(CC2)c2nc(N3CCCC(c4ncc(Cl)cn4)CC3)nc(NC3(CO)CCC3)c21]

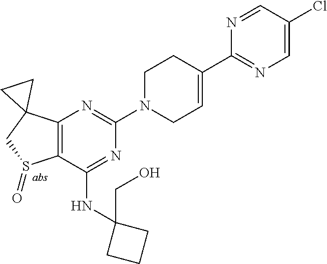 [SMILES: O=[S@@]1CC2(CC2)c2nc(N3CC=C(c4ncc(Cl)cn4)CC3)nc(NC3(CO)CCC3)c21]

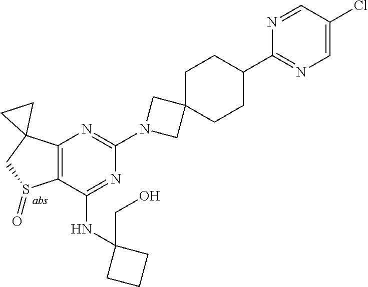 [SMILES: O=[S@@]1CC2(CC2)c2nc(N3CC4(CCC(c5ncc(Cl)cn5)CC4)C3)nc(NC3(CO)CCC3)c21]

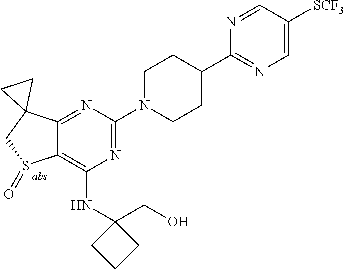

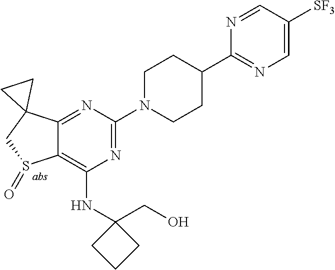

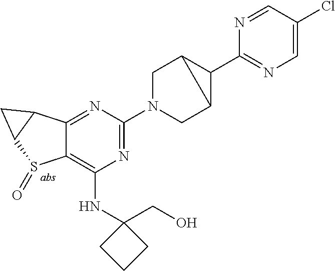

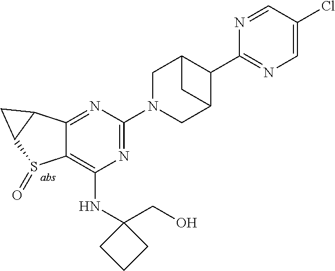

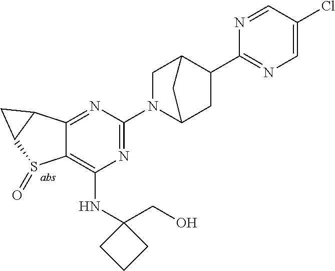

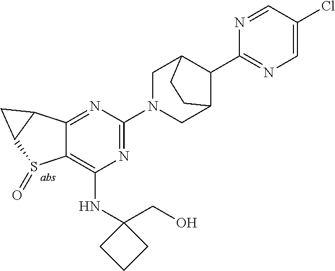

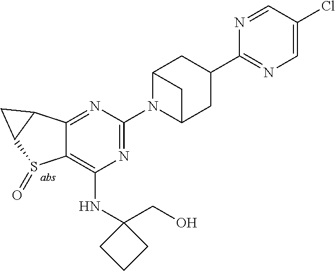

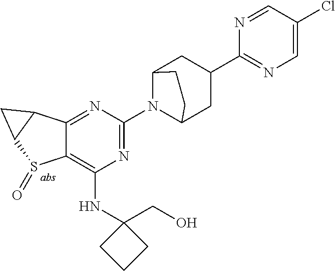

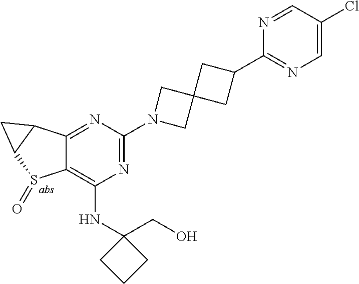

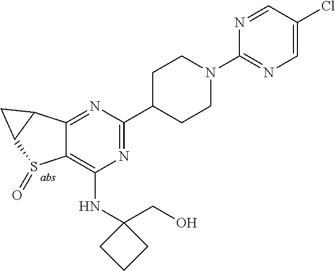

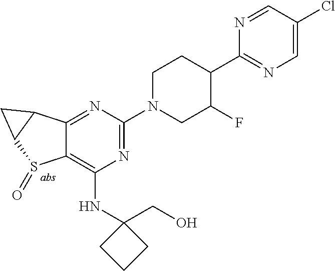

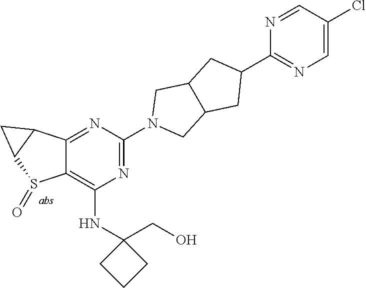

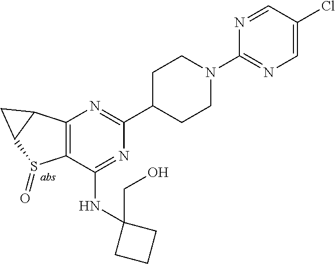

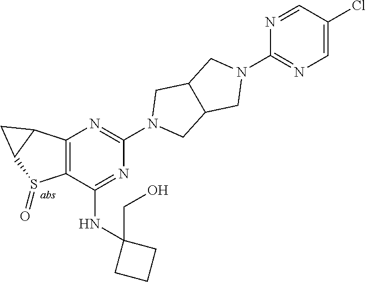

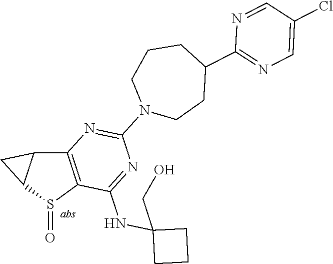

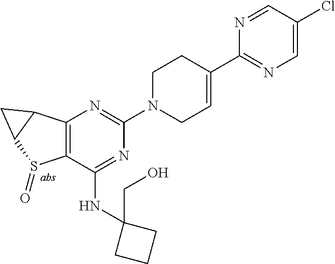

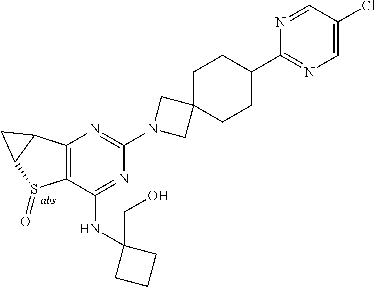

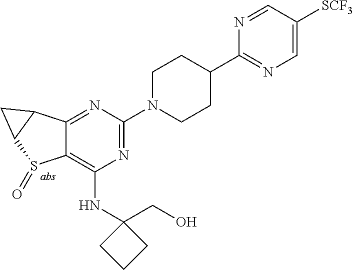

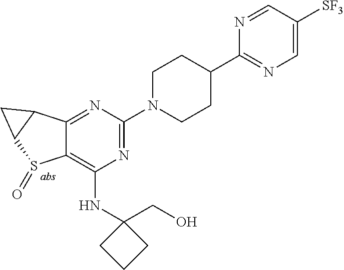

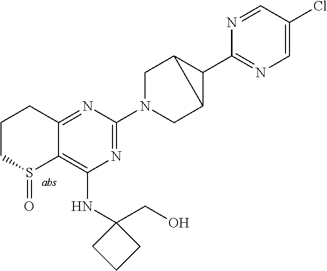 [SMILES: O=[S@@]1CCCc2nc(N3CC4C(C3)C4c3ncc(Cl)cn3)nc(NC3(CO)CCC3)c21]

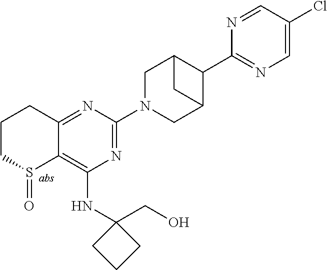 [SMILES: O=[S@@]1CCCc2nc(N3CC4CC(C3)C4c3ncc(Cl)cn3)nc(NC3(CO)CCC3)c21]

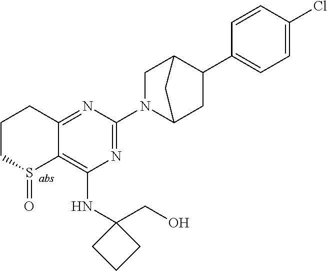 [SMILES: O=[S@@]1CCCc2nc(N3CC4CC3CC4c3ccc(Cl)cc3)nc(NC3(CO)CCC3)c21]

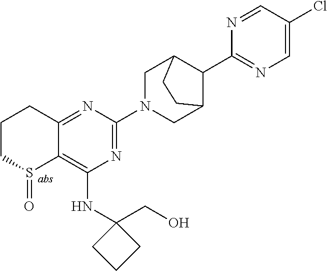 [SMILES: O=[S@@]1CCCc2nc(N3CC4CCC(C3)C4c3ncc(Cl)cn3)nc(NC3(CO)CCC3)c21]

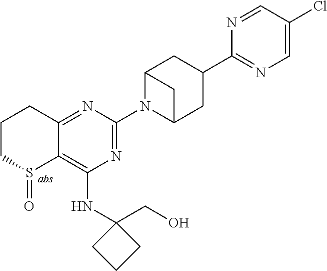 [SMILES: O=[S@@]1CCCc2nc(N3C4CC(c5ncc(Cl)cn5)CC3C4)nc(NC3(CO)CCC3)c21]

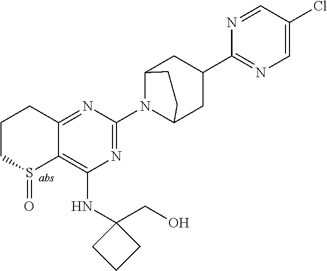 [SMILES: O=[S@@]1CCCc2nc(N3C4CCC3CC(c3ncc(Cl)cn3)C4)nc(NC3(CO)CCC3)c21]

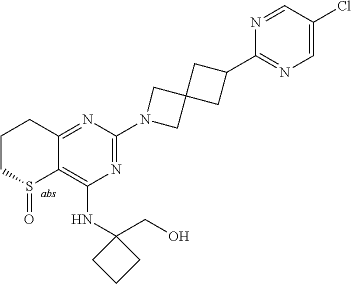 [SMILES: O=[S@@]1CCCc2nc(N3CC4(CC(c5ncc(Cl)cn5)C4)C3)nc(NC3(CO)CCC3)c21]

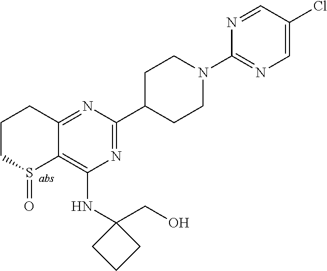 [SMILES: O=[S@@]1CCCc2nc(C3CCN(c4ncc(Cl)cn4)CC3)nc(NC3(CO)CCC3)c21]

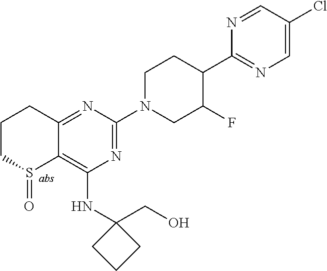 [SMILES: O=[S@@]1CCCc2nc(N3CCC(c4ncc(Cl)cn4)C(F)C3)nc(NC3(CO)CCC3)c21]

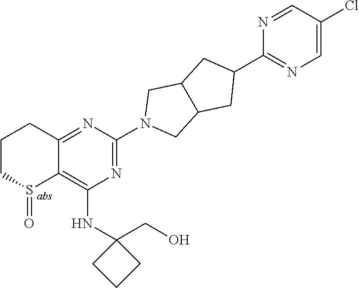 [SMILES: O=[S@@]1CCCc2nc(N3CC4CC(c5ncc(Cl)cn5)CC4C3)nc(NC3(CO)CCC3)c21]

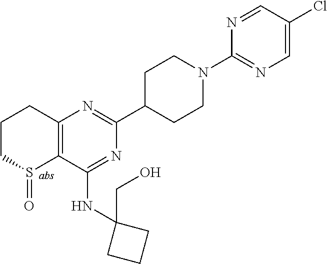 [SMILES: O=[S@@]1CCCc2nc(C3CCN(c4ncc(Cl)cn4)CC3)nc(NC3(CO)CCC3)c21]

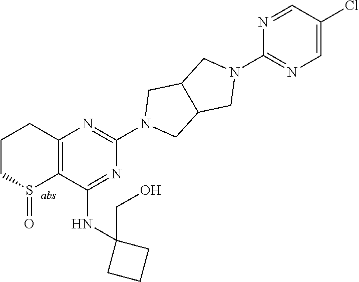 [SMILES: O=[S@@]1CCCc2nc(N3CC4CN(c5ncc(Cl)cn5)CC4C3)nc(NC3(CO)CCC3)c21]

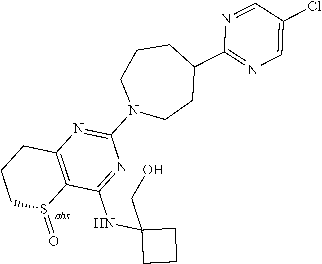 [SMILES: O=[S@@]1CCCc2nc(N3CCCC(c4ncc(Cl)cn4)CC3)nc(NC3(CO)CCC3)c21]

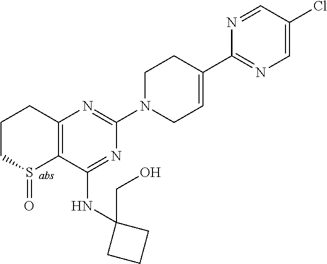 [SMILES: O=[S@@]1CCCc2nc(N3CC=C(c4ncc(Cl)cn4)CC3)nc(NC3(CO)CCC3)c21]

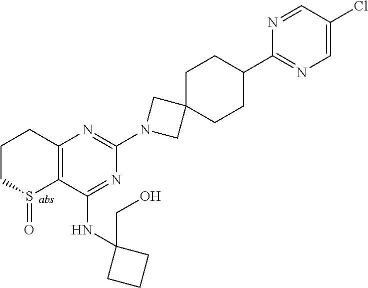 [SMILES: O=[S@@]1CCCc2nc(N3CC4(CCC(c5ncc(Cl)cn5)CC4)C3)nc(NC3(CO)CCC3)c21]

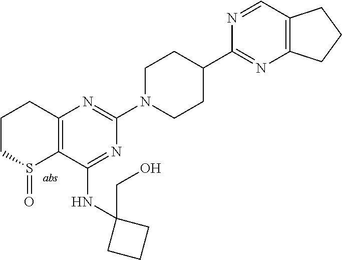 [SMILES: O=[S@@]1CCCc2nc(N3CCC(c4ncc5c(n4)CCC5)CC3)nc(NC3(CO)CCC3)c21]

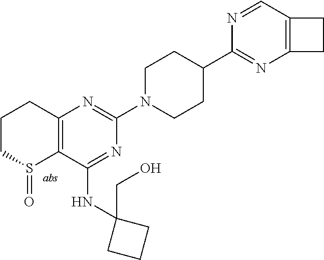 [SMILES: O=[S@@]1CCCc2nc(N3CCC(c4ncc5c(n4)CC5)CC3)nc(NC3(CO)CCC3)c21]

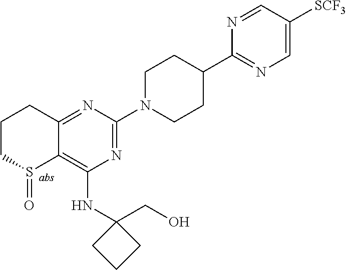

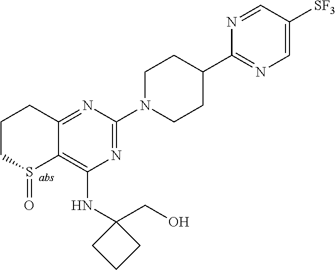

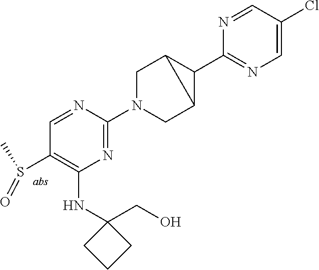 [SMILES: C[S@@](=O)c1cnc(N2CC3C(C2)C3c2ncc(Cl)cn2)nc1NC1(CO)CCC1]

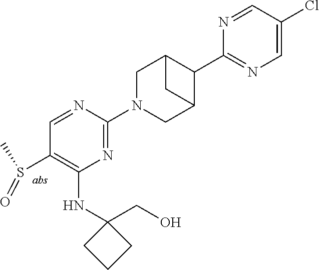 [SMILES: C[S@@](=O)c1cnc(N2CC3CC(C2)C3c2ncc(Cl)cn2)nc1NC1(CO)CCC1]

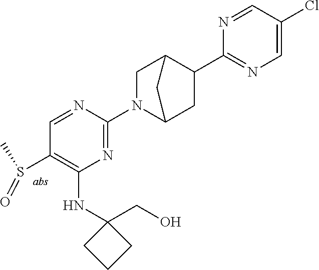 [SMILES: C[S@@](=O)c1cnc(N2CC3CC2CC3c2ncc(Cl)cn2)nc1NC1(CO)CCC1]

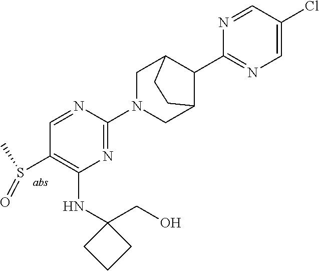 [SMILES: C[S@@](=O)c1cnc(N2CC3CCC(C2)C3c2ncc(Cl)cn2)nc1NC1(CO)CCC1]

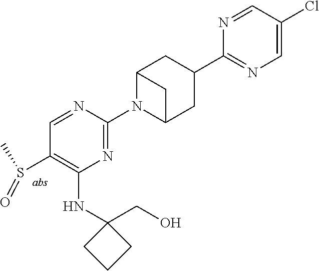 [SMILES: C[S@@](=O)c1cnc(N2C3CC(c4ncc(Cl)cn4)CC2C3)nc1NC1(CO)CCC1]

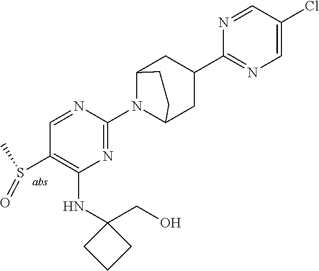 [SMILES: C[S@@](=O)c1cnc(N2C3CCC2CC(c2ncc(Cl)cn2)C3)nc1NC1(CO)CCC1]

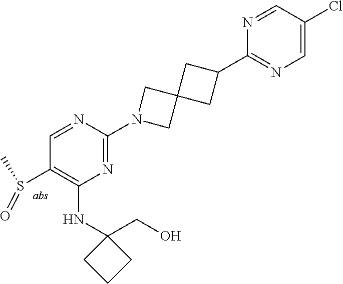 [SMILES: C[S@@](=O)c1cnc(N2CC3(CC(c4ncc(Cl)cn4)C3)C2)nc1NC1(CO)CCC1]

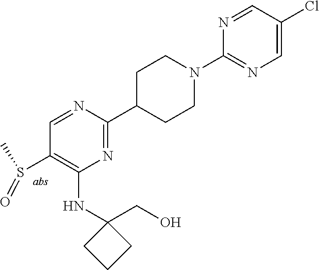 [SMILES: C[S@@](=O)c1cnc(C2CCN(c3ncc(Cl)cn3)CC2)nc1NC1(CO)CCC1]

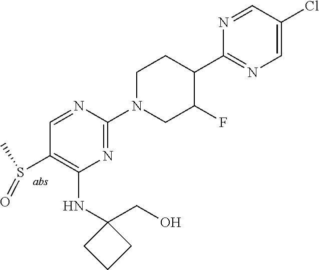 [SMILES: C[S@@](=O)c1cnc(N2CCC(c3ncc(Cl)cn3)C(F)C2)nc1NC1(CO)CCC1]

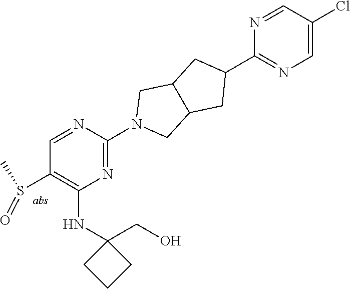 [SMILES: C[S@@](=O)c1cnc(N2CC3CC(c4ncc(Cl)cn4)CC3C2)nc1NC1(CO)CCC1]

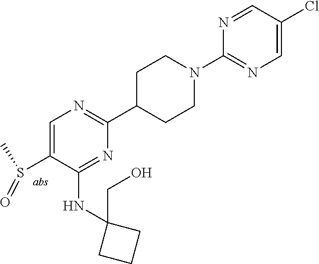 [SMILES: C[S@@](=O)c1cnc(C2CCN(c3ncc(Cl)cn3)CC2)nc1NC1(CO)CCC1]

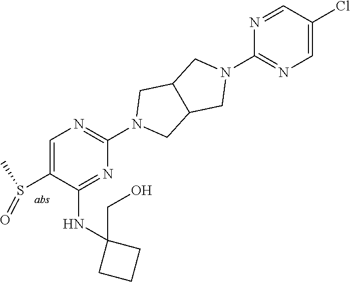 [SMILES: C[S@@](=O)c1cnc(N2CC3CN(c4ncc(Cl)cn4)CC3C2)nc1NC1(CO)CCC1]

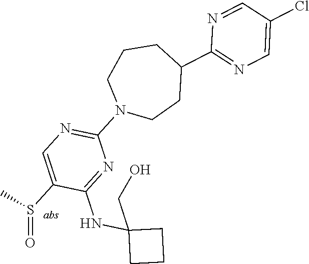 [SMILES: C[S@@](=O)c1cnc(N2CCCC(c3ncc(Cl)cn3)CC2)nc1NC1(CO)CCC1]

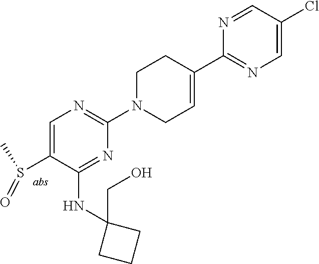 [SMILES: C[S@@](=O)c1cnc(N2CC=C(c3ncc(Cl)cn3)CC2)nc1NC1(CO)CCC1]

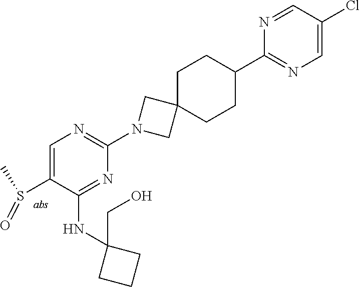 [SMILES: C[S@@](=O)c1cnc(N2CC3(CCC(c4ncc(Cl)cn4)CC3)C2)nc1NC1(CO)CCC1]

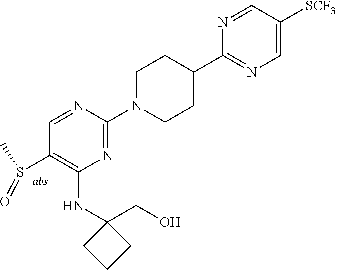

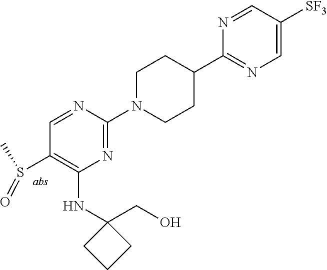

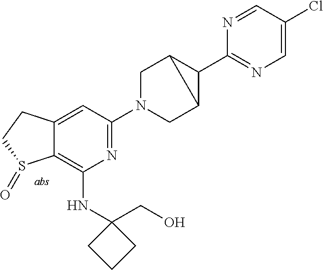 [SMILES: O=[S@@]1CCc2cc(N3CC4C(C3)C4c3ncc(Cl)cn3)nc(NC3(CO)CCC3)c21]

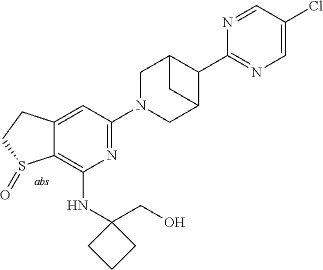 [SMILES: O=[S@@]1CCc2cc(N3CC4CC(C3)C4c3ncc(Cl)cn3)nc(NC3(CO)CCC3)c21]

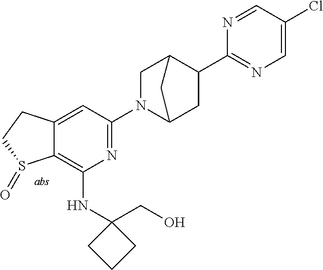 [SMILES: O=[S@@]1CCc2cc(N3CC4CC3CC4c3ncc(Cl)cn3)nc(NC3(CO)CCC3)c21]

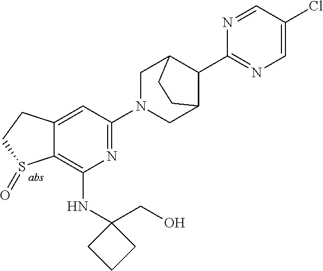 [SMILES: O=[S@@]1CCc2cc(N3CC4CCC(C3)C4c3ncc(Cl)cn3)nc(NC3(CO)CCC3)c21]

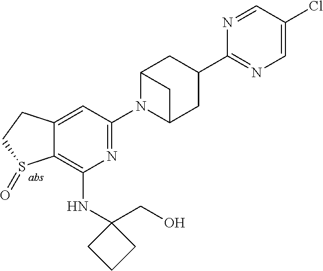 [SMILES: O=[S@@]1CCc2cc(N3C4CC(c5ncc(Cl)cn5)CC3C4)nc(NC3(CO)CCC3)c21]

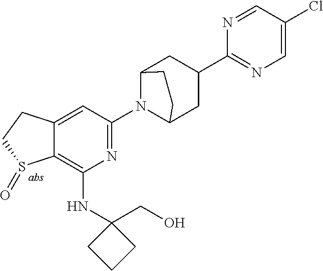 [SMILES: O=[S@@]1CCc2cc(N3C4CCC3CC(c3ncc(Cl)cn3)C4)nc(NC3(CO)CCC3)c21]

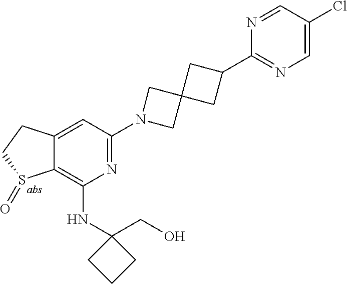 [SMILES: O=[S@@]1CCc2cc(N3CC4(CC(c5ncc(Cl)cn5)C4)C3)nc(NC3(CO)CCC3)c21]

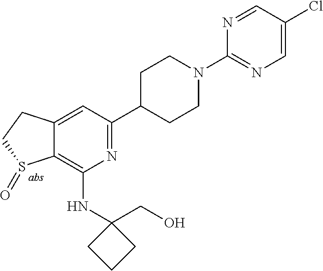 [SMILES: O=[S@@]1CCc2cc(C3CCN(c4ncc(Cl)cn4)CC3)nc(NC3(CO)CCC3)c21]

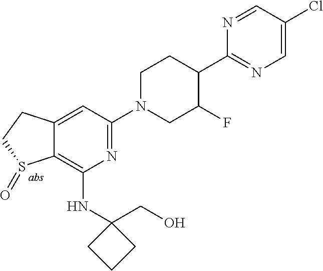 [SMILES: O=[S@@]1CCc2cc(N3CCC(c4ncc(Cl)cn4)C(F)C3)nc(NC3(CO)CCC3)c21]

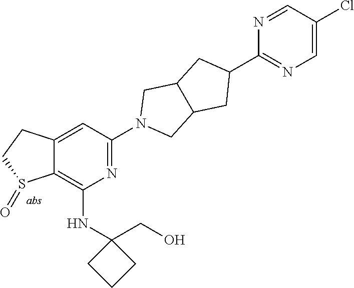 [SMILES: O=[S@@]1CCc2cc(N3CC4CC(c5ncc(Cl)cn5)CC4C3)nc(NC3(CO)CCC3)c21]

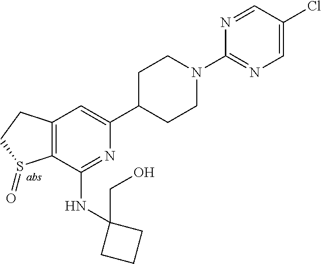 [SMILES: O=[S@@]1CCc2cc(C3CCN(c4ncc(Cl)cn4)CC3)nc(NC3(CO)CCC3)c21]

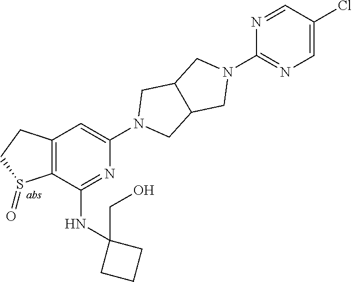 [SMILES: O=[S@@]1CCc2cc(N3CC4CN(c5ncc(Cl)cn5)CC4C3)nc(NC3(CO)CCC3)c21]

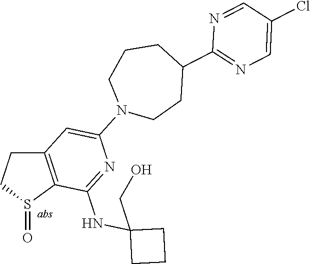 [SMILES: O=[S@@]1CCc2cc(N3CCCC(c4ncc(Cl)cn4)CC3)nc(NC3(CO)CCC3)c21]

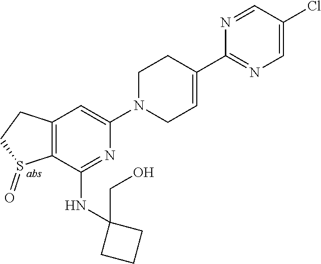 [SMILES: O=[S@@]1CCc2cc(N3CC=C(c4ncc(Cl)cn4)CC3)nc(NC3(CO)CCC3)c21]

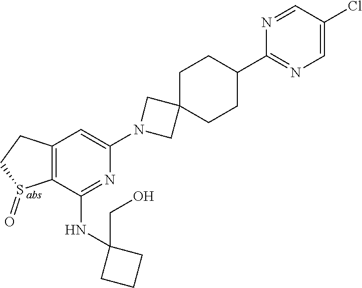 [SMILES: O=[S@@]1CCc2cc(N3CC4(CCC(c5ncc(Cl)cn5)CC4)C3)nc(NC3(CO)CCC3)c21]

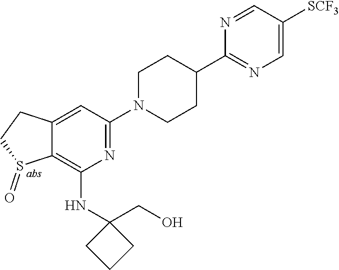

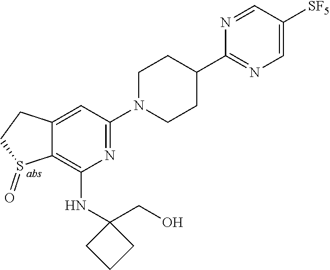

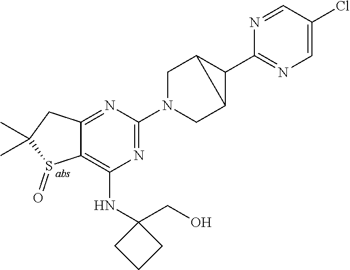 [SMILES: CC1(C)Cc2nc(N3CC4C(C3)C4c3ncc(Cl)cn3)nc(NC3(CO)CCC3)c2[S@@]1=O]

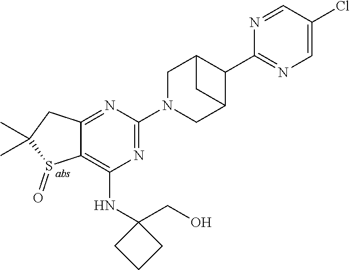 [SMILES: CC1(C)Cc2nc(N3CC4CC(C3)C4c3ncc(Cl)cn3)nc(NC3(CO)CCC3)c2[S@@]1=O]

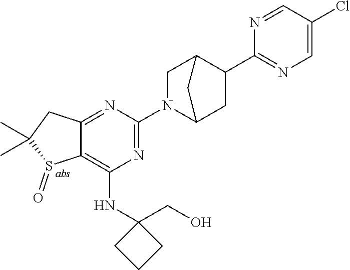 [SMILES: CC1(C)Cc2nc(N3CC4CC3CC4c3ncc(Cl)cn3)nc(NC3(CO)CCC3)c2[S@@]1=O]

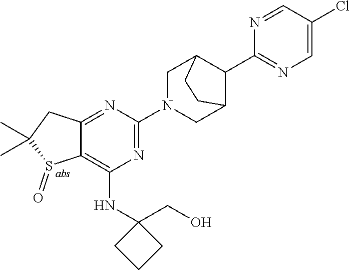 [SMILES: CC1(C)Cc2nc(N3CC4CCC(C3)C4c3ncc(Cl)cn3)nc(NC3(CO)CCC3)c2[S@@]1=O]

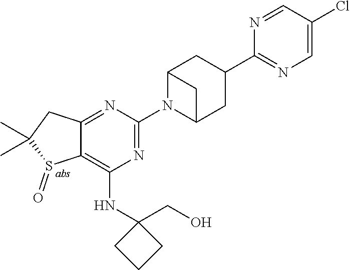 [SMILES: CC1(C)Cc2nc(N3C4CC(c5ncc(Cl)cn5)CC3C4)nc(NC3(CO)CCC3)c2[S@@]1=O]

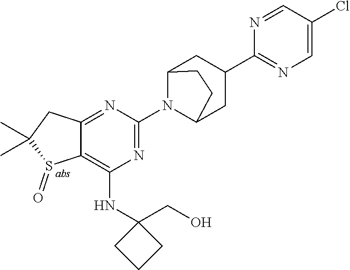 [SMILES: CC1(C)Cc2nc(N3C4CCC3CC(c3ncc(Cl)cn3)C4)nc(NC3(CO)CCC3)c2[S@@]1=O]

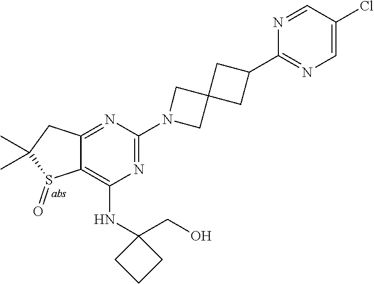 [SMILES: CC1(C)Cc2nc(N3CC4(CC(c5ncc(Cl)cn5)C4)C3)nc(NC3(CO)CCC3)c2[S@@]1=O]

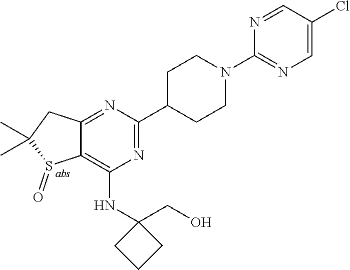 [SMILES: CC1(C)Cc2nc(C3CCN(c4ncc(Cl)cn4)CC3)nc(NC3(CO)CCC3)c2[S@@]1=O]

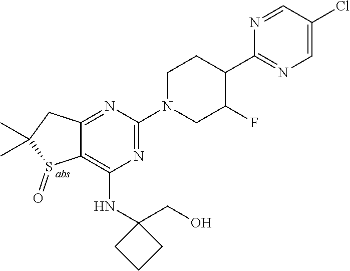 [SMILES: CC1(C)Cc2nc(N3CCC(c4ncc(Cl)cn4)C(F)C3)nc(NC3(CO)CCC3)c2[S@@]1=O]

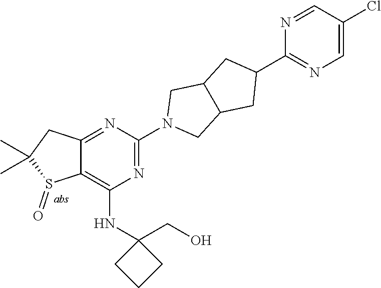 [SMILES: CC1(C)Cc2nc(N3CC4CC(c5ncc(Cl)cn5)CC4C3)nc(NC3(CO)CCC3)c2[S@@]1=O]

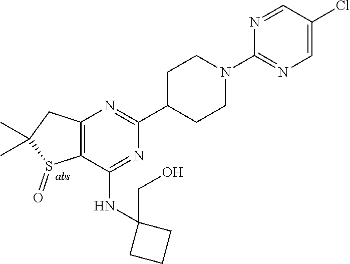 [SMILES: CC1(C)Cc2nc(C3CCN(c4ncc(Cl)cn4)CC3)nc(NC3(CO)CCC3)c2[S@@]1=O]

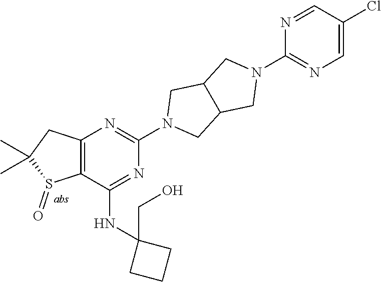 [SMILES: CC1(C)Cc2nc(N3CC4CN(c5ncc(Cl)cn5)CC4C3)nc(NC3(CO)CCC3)c2[S@@]1=O]

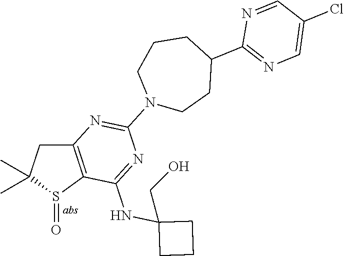 [SMILES: CC1(C)Cc2nc(N3CCCC(c4ncc(Cl)cn4)CC3)nc(NC3(CO)CCC3)c2[S@@]1=O]

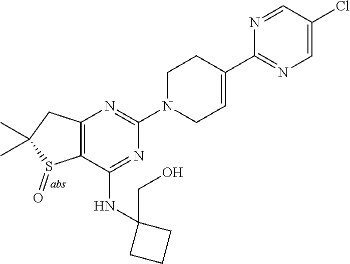

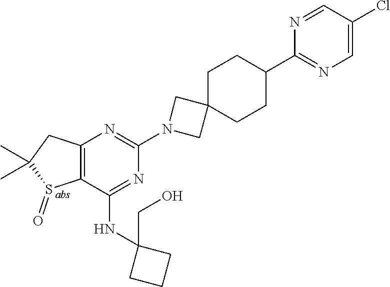 [SMILES: CC1(C)Cc2nc(N3CC4(CCC(c5ncc(Cl)cn5)CC4)C3)nc(NC3(CO)CCC3)c2[S@@]1=O]

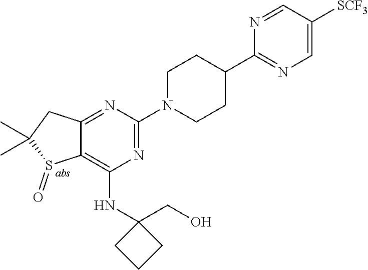

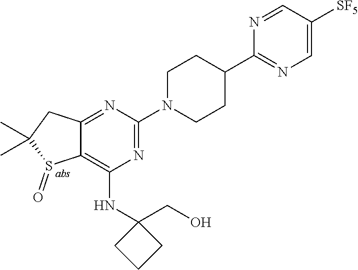

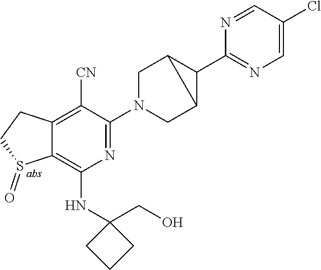 [SMILES: N#Cc1c(N2CC3C(C2)C3c2ncc(Cl)cn2)nc(NC2(CO)CCC2)c2c1CC[S@]2=O]

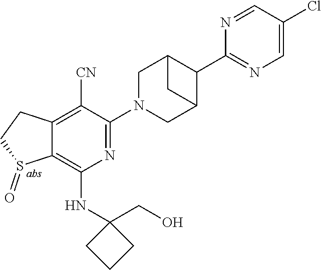 [SMILES: N#Cc1c(N2CC3CC(C2)C3c2ncc(Cl)cn2)nc(NC2(CO)CCC2)c2c1CC[S@]2=O]

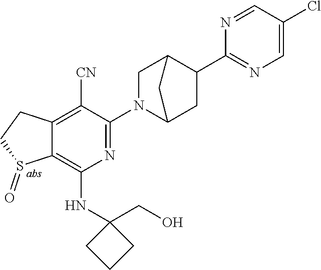 [SMILES: N#Cc1c(N2CC3CC2CC3c2ncc(Cl)cn2)nc(NC2(CO)CCC2)c2c1CC[S@]2=O]

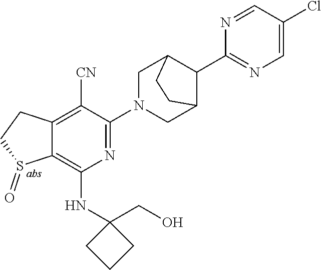 [SMILES: N#Cc1c(N2CC3CCC(C2)C3c2ncc(Cl)cn2)nc(NC2(CO)CCC2)c2c1CC[S@]2=O]

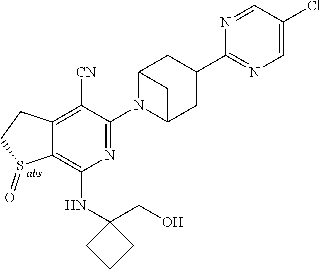 [SMILES: N#Cc1c(N2C3CC(c4ncc(Cl)cn4)CC2C3)nc(NC2(CO)CCC2)c2c1CC[S@]2=O]

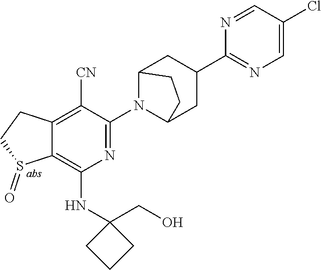 [SMILES: N#Cc1c(N2C3CCC2CC(c2ncc(Cl)cn2)C3)nc(NC2(CO)CCC2)c2c1CC[S@]2=O]

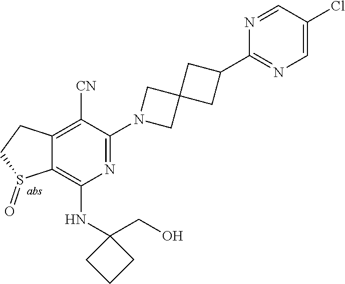 [SMILES: N#Cc1c(N2CC3(CC(c4ncc(Cl)cn4)C3)C2)nc(NC2(CO)CCC2)c2c1CC[S@]2=O]

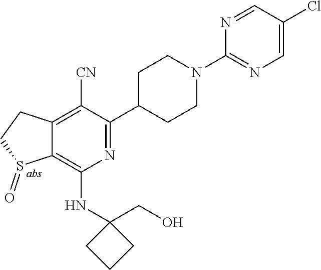 [SMILES: N#Cc1c(C2CCN(c3ncc(Cl)cn3)CC2)nc(NC2(CO)CCC2)c2c1CC[S@]2=O]

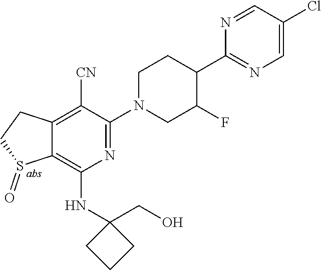 [SMILES: N#Cc1c(N2CCC(c3ncc(Cl)cn3)C(F)C2)nc(NC2(CO)CCC2)c2c1CC[S@]2=O]

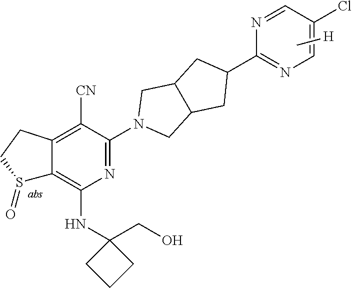

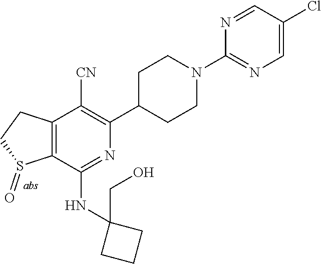 [SMILES: N#Cc1c(C2CCN(c3ncc(Cl)cn3)CC2)nc(NC2(CO)CCC2)c2c1CC[S@]2=O]

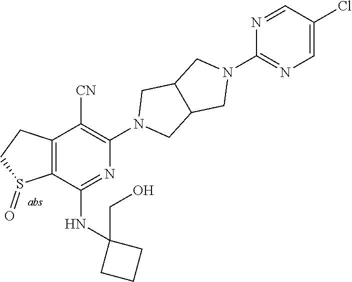 [SMILES: N#Cc1c(N2CC3CN(c4ncc(Cl)cn4)CC3C2)nc(NC2(CO)CCC2)c2c1CC[S@]2=O]

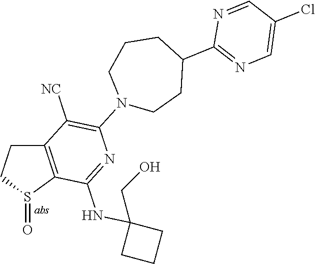 [SMILES: N#Cc1c(N2CCCC(c3ncc(Cl)cn3)CC2)nc(NC2(CO)CCC2)c2c1CC[S@]2=O]

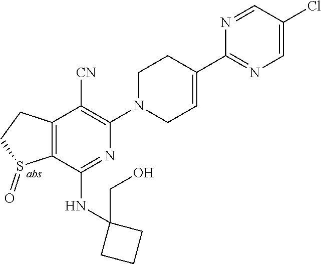 [SMILES: N#Cc1c(N2CC=C(c3ncc(Cl)cn3)CC2)nc(NC2(CO)CCC2)c2c1CC[S@]2=O]

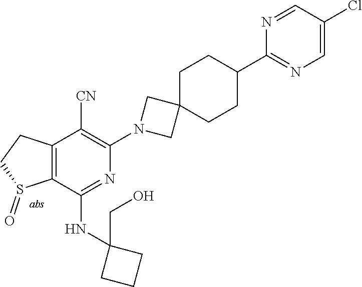 [SMILES: N#Cc1c(N2CC3(CCC(c4ncc(Cl)cn4)CC3)C2)nc(NC2(CO)CCC2)c2c1CC[S@]2=O]

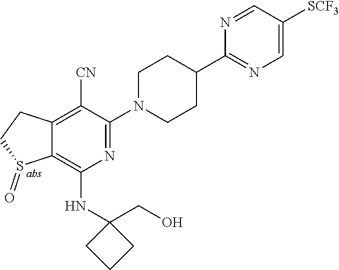

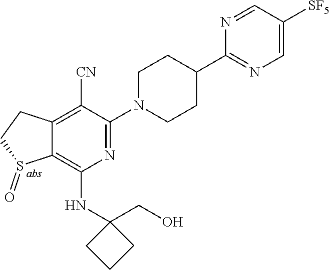

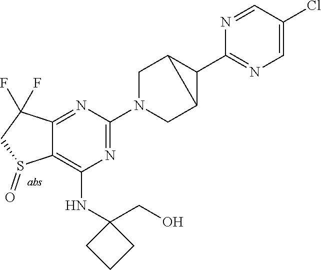 [SMILES: O=[S@@]1CC(F)(F)c2nc(N3CC4C(C3)C4c3ncc(Cl)cn3)nc(NC3(CO)CCC3)c21]

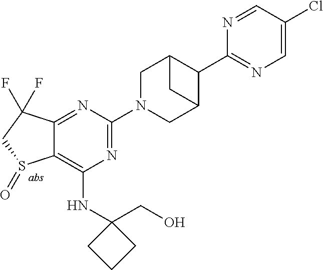 [SMILES: O=[S@@]1CC(F)(F)c2nc(N3CC4CC(C3)C4c3ncc(Cl)cn3)nc(NC3(CO)CCC3)c21]

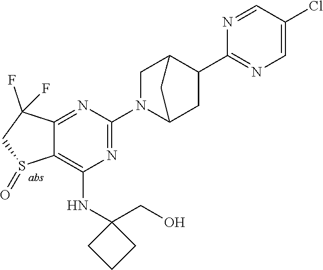 [SMILES: O=[S@@]1CC(F)(F)c2nc(N3CC4CC3CC4c3ncc(Cl)cn3)nc(NC3(CO)CCC3)c21]

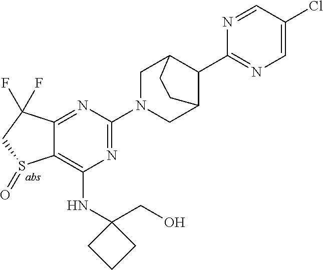 [SMILES: O=[S@@]1CC(F)(F)c2nc(N3CC4CCC(C3)C4c3ncc(Cl)cn3)nc(NC3(CO)CCC3)c21]

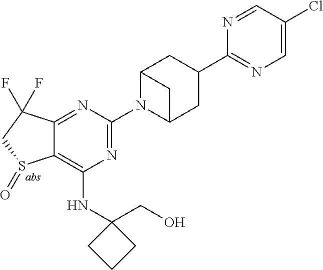 [SMILES: O=[S@@]1CC(F)(F)c2nc(N3C4CC(c5ncc(Cl)cn5)CC3C4)nc(NC3(CO)CCC3)c21]

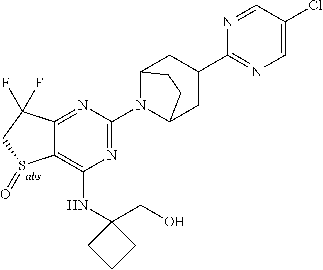 [SMILES: O=[S@@]1CC(F)(F)c2nc(N3C4CCC3CC(c3ncc(Cl)cn3)C4)nc(NC3(CO)CCC3)c21]

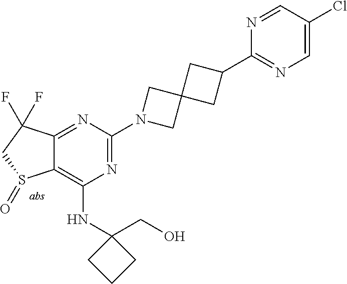 [SMILES: O=[S@@]1CC(F)(F)c2nc(N3CC4(CC(c5ncc(Cl)cn5)C4)C3)nc(NC3(CO)CCC3)c21]

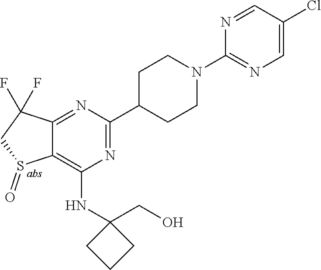 [SMILES: O=[S@@]1CC(F)(F)c2nc(C3CCN(c4ncc(Cl)cn4)CC3)nc(NC3(CO)CCC3)c21]

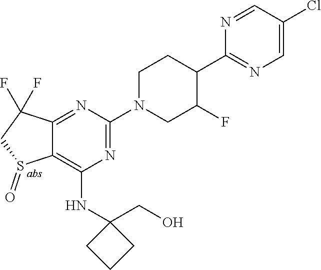 [SMILES: O=[S@@]1CC(F)(F)c2nc(N3CCC(c4ncc(Cl)cn4)C(F)C3)nc(NC3(CO)CCC3)c21]

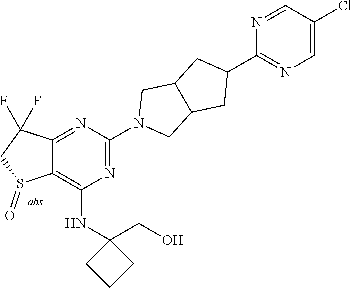 [SMILES: O=[S@@]1CC(F)(F)c2nc(N3CC4CC(c5ncc(Cl)cn5)CC4C3)nc(NC3(CO)CCC3)c21]

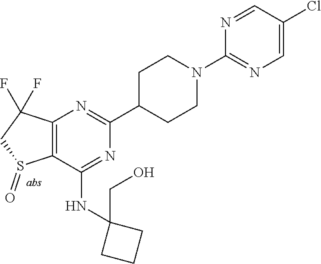 [SMILES: O=[S@@]1CC(F)(F)c2nc(C3CCN(c4ncc(Cl)cn4)CC3)nc(NC3(CO)CCC3)c21]

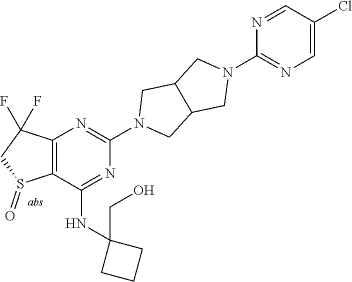 [SMILES: O=[S@@]1CC(F)(F)c2nc(N3CC4CN(c5ncc(Cl)cn5)CC4C3)nc(NC3(CO)CCC3)c21]

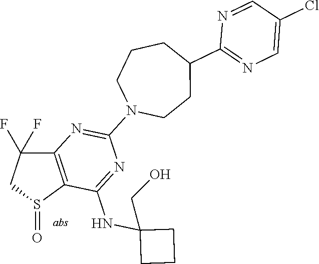 [SMILES: O=[S@@]1CC(F)(F)c2nc(N3CCCC(c4ncc(Cl)cn4)CC3)nc(NC3(CO)CCC3)c21]

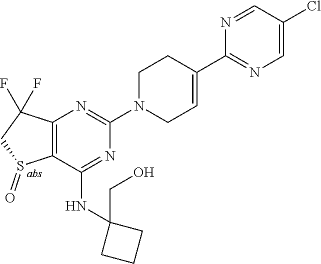 [SMILES: O=[S@@]1CC(F)(F)c2nc(N3CC=C(c4ncc(Cl)cn4)CC3)nc(NC3(CO)CCC3)c21]

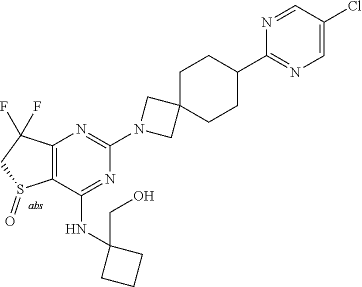 [SMILES: O=[S@@]1CC(F)(F)c2nc(N3CC4(CCC(c5ncc(Cl)cn5)CC4)C3)nc(NC3(CO)CCC3)c21]

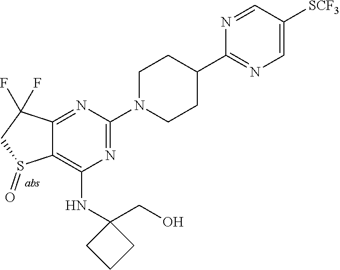

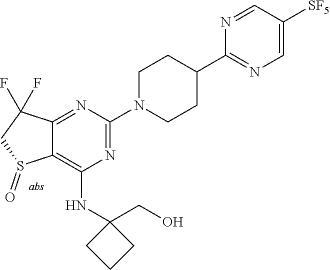

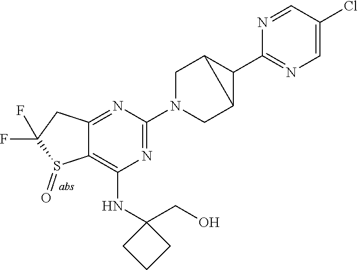 [SMILES: O=[S@]1c2c(nc(N3CC4C(C3)C4c3ncc(Cl)cn3)nc2NC2(CO)CCC2)CC1(F)F]

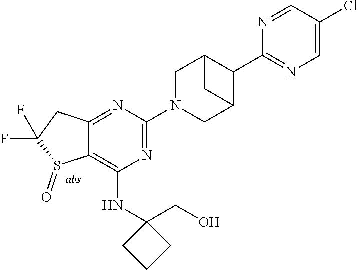 [SMILES: O=[S@]1c2c(nc(N3CC4CC(C3)C4c3ncc(Cl)cn3)nc2NC2(CO)CCC2)CC1(F)F]

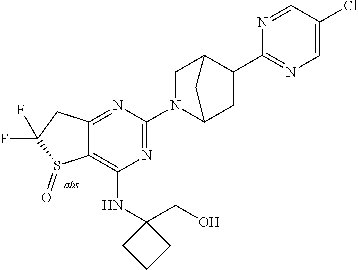 [SMILES: O=[S@]1c2c(nc(N3CC4CC3CC4c3ncc(Cl)cn3)nc2NC2(CO)CCC2)CC1(F)F]

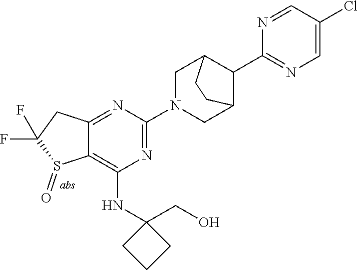 [SMILES: O=[S@]1c2c(nc(N3CC4CCC(C3)C4c3ncc(Cl)cn3)nc2NC2(CO)CCC2)CC1(F)F]

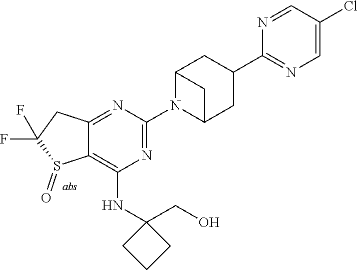 [SMILES: O=[S@]1c2c(nc(N3C4CC(c5ncc(Cl)cn5)CC3C4)nc2NC2(CO)CCC2)CC1(F)F]

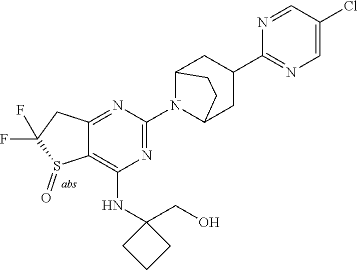 [SMILES: O=[S@]1c2c(nc(N3C4CCC3CC(c3ncc(Cl)cn3)C4)nc2NC2(CO)CCC2)CC1(F)F]

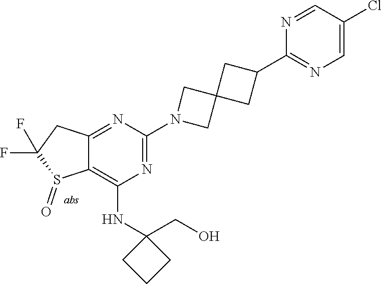 [SMILES: O=[S@]1c2c(nc(N3CC4(CC(c5ncc(Cl)cn5)C4)C3)nc2NC2(CO)CCC2)CC1(F)F]

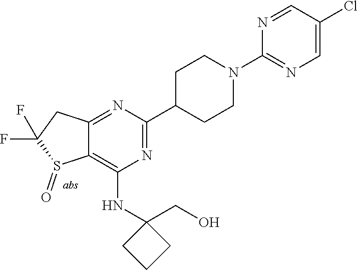 [SMILES: O=[S@]1c2c(nc(C3CCN(c4ncc(Cl)cn4)CC3)nc2NC2(CO)CCC2)CC1(F)F]

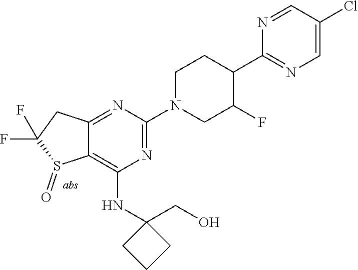 [SMILES: O=[S@]1c2c(nc(N3CCC(c4ncc(Cl)cn4)C(F)C3)nc2NC2(CO)CCC2)CC1(F)F]

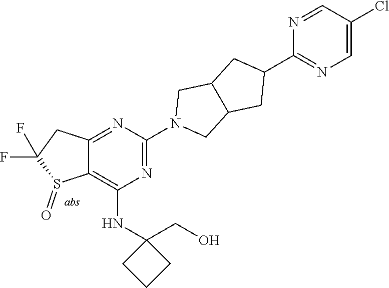 [SMILES: O=[S@]1c2c(nc(N3CC4CC(c5ncc(Cl)cn5)CC4C3)nc2NC2(CO)CCC2)CC1(F)F]

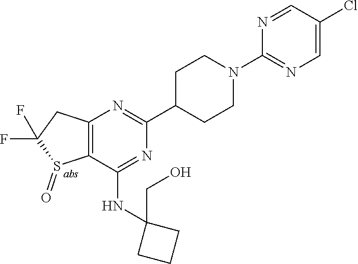 [SMILES: O=[S@]1c2c(nc(C3CCN(c4ncc(Cl)cn4)CC3)nc2NC2(CO)CCC2)CC1(F)F]

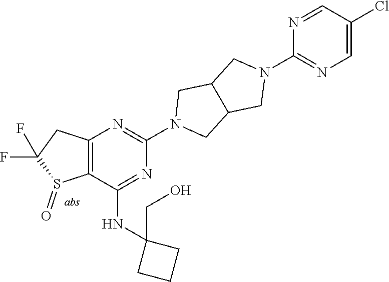 [SMILES: O=[S@]1c2c(nc(N3CC4CN(c5ncc(Cl)cn5)CC4C3)nc2NC2(CO)CCC2)CC1(F)F]

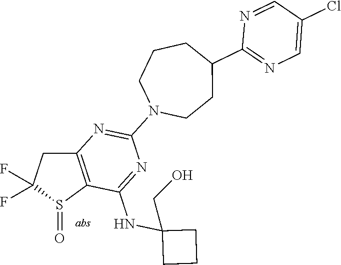 [SMILES: O=[S@]1c2c(nc(N3CCCC(c4ncc(Cl)cn4)CC3)nc2NC2(CO)CCC2)CC1(F)F]

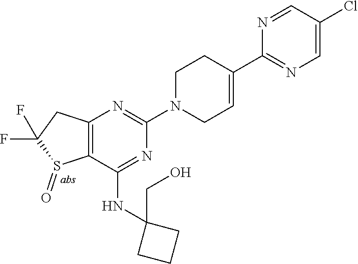 [SMILES: O=[S@]1c2c(nc(N3CC=C(c4ncc(Cl)cn4)CC3)nc2NC2(CO)CCC2)CC1(F)F]

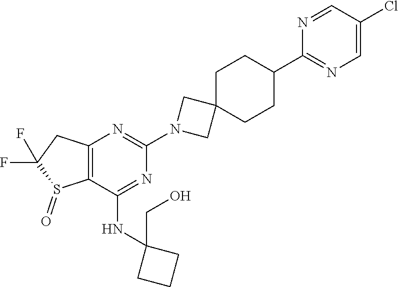 [SMILES: O=[S@]1c2c(nc(N3CC4(CCC(c5ncc(Cl)cn5)CC4)C3)nc2NC2(CO)CCC2)CC1(F)F]

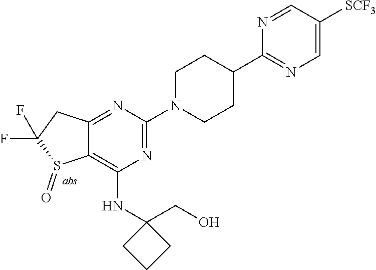

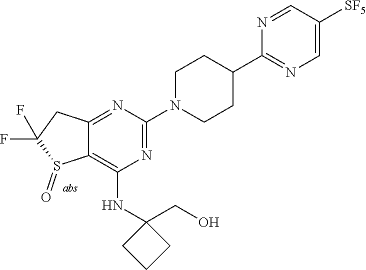

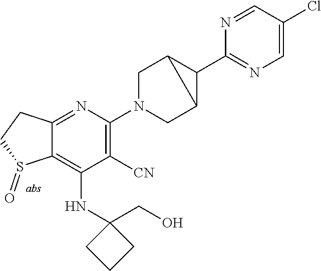 [SMILES: N#Cc1c(N2CC3C(C2)C3c2ncc(Cl)cn2)nc2c(c1NC1(CO)CCC1)[S@](=O)CC2]

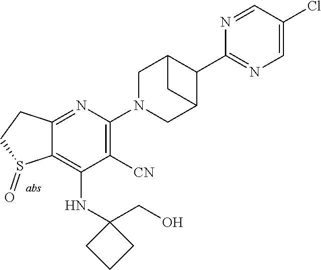 [SMILES: N#Cc1c(N2CC3CC(C2)C3c2ncc(Cl)cn2)nc2c(c1NC1(CO)CCC1)[S@](=O)CC2]

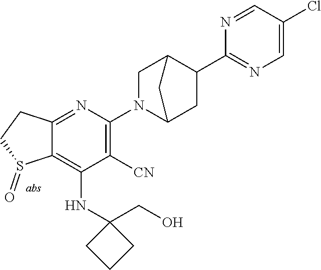 [SMILES: N#Cc1c(N2CC3CC2CC3c2ncc(Cl)cn2)nc2c(c1NC1(CO)CCC1)[S@](=O)CC2]

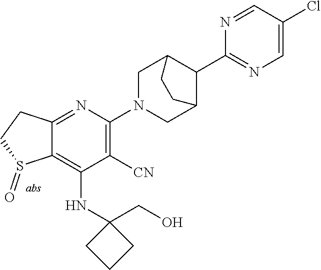 [SMILES: N#Cc1c(N2CC3CCC(C2)C3c2ncc(Cl)cn2)nc2c(c1NC1(CO)CCC1)[S@](=O)CC2]

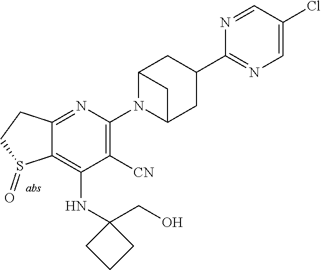 [SMILES: N#Cc1c(N2C3CC(c4ncc(Cl)cn4)CC2C3)nc2c(c1NC1(CO)CCC1)[S@](=O)CC2]

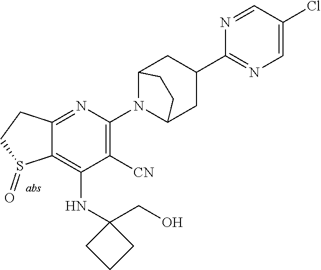 [SMILES: N#Cc1c(N2C3CCC2CC(c2ncc(Cl)cn2)C3)nc2c(c1NC1(CO)CCC1)[S@](=O)CC2]

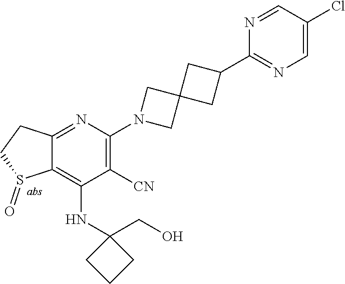 [SMILES: N#Cc1c(N2CC3(CC(c4ncc(Cl)cn4)C3)C2)nc2c(c1NC1(CO)CCC1)[S@](=O)CC2]

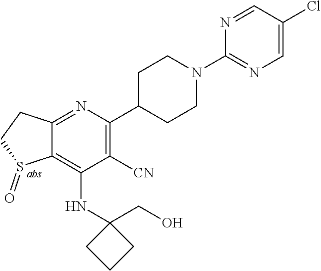 [SMILES: N#Cc1c(C2CCN(c3ncc(Cl)cn3)CC2)nc2c(c1NC1(CO)CCC1)[S@](=O)CC2]

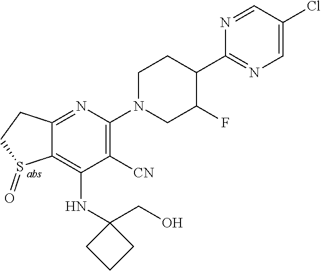 [SMILES: N#Cc1c(N2CCC(c3ncc(Cl)cn3)C(F)C2)nc2c(c1NC1(CO)CCC1)[S@](=O)CC2]

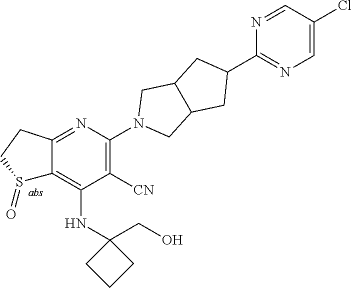 [SMILES: N#Cc1c(N2CC3CC(c4ncc(Cl)cn4)CC3C2)nc2c(c1NC1(CO)CCC1)[S@](=O)CC2]

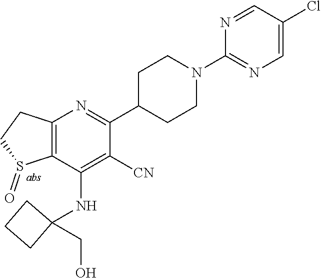 [SMILES: N#Cc1c(C2CCN(c3ncc(Cl)cn3)CC2)nc2c(c1NC1(CO)CCC1)[S@](=O)CC2]

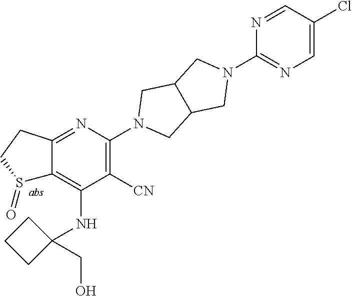 [SMILES: N#Cc1c(N2CC3CN(c4ncc(Cl)cn4)CC3C2)nc2c(c1NC1(CO)CCC1)[S@](=O)CC2]

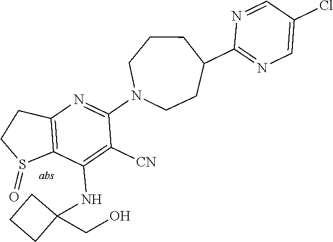 [SMILES: N#Cc1c(N2CCCC(c3ncc(Cl)cn3)CC2)nc2c(c1NC1(CO)CCC1)S(=O)CC2]

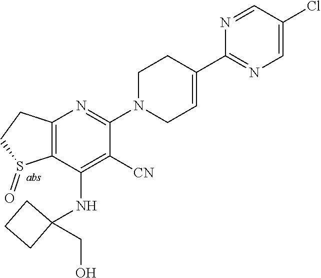 [SMILES: N#Cc1c(N2CC=C(c3ncc(Cl)cn3)CC2)nc2c(c1NC1(CO)CCC1)[S@](=O)CC2]

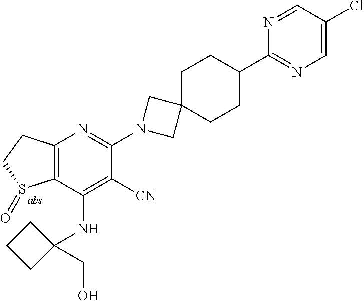 [SMILES: N#Cc1c(N2CC3(CCC(c4ncc(Cl)cn4)CC3)C2)nc2c(c1NC1(CO)CCC1)[S@](=O)CC2]

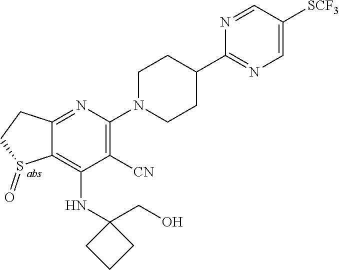

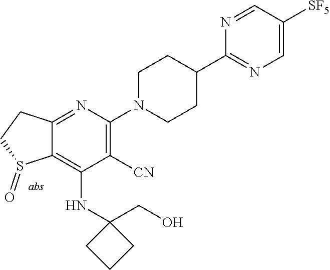

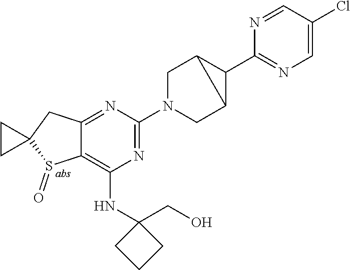 [SMILES: O=[S@]1c2c(nc(N3CC4C(C3)C4c3ncc(Cl)cn3)nc2NC2(CO)CCC2)CC12CC2]

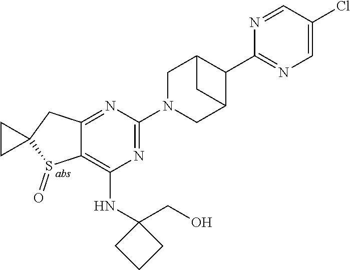 [SMILES: O=[S@]1c2c(nc(N3CC4CC(C3)C4c3ncc(Cl)cn3)nc2NC2(CO)CCC2)CC12CC2]

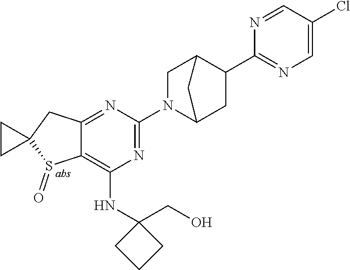 [SMILES: O=[S@]1c2c(nc(N3CC4CC3CC4c3ncc(Cl)cn3)nc2NC2(CO)CCC2)CC12CC2]

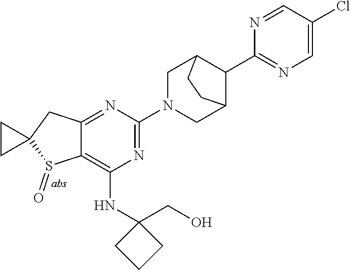 [SMILES: O=[S@]1c2c(nc(N3CC4CCC(C3)C4c3ncc(Cl)cn3)nc2NC2(CO)CCC2)CC12CC2]

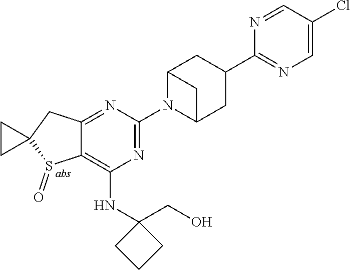 [SMILES: O=[S@]1c2c(nc(N3C4CC(c5ncc(Cl)cn5)CC3C4)nc2NC2(CO)CCC2)CC12CC2]

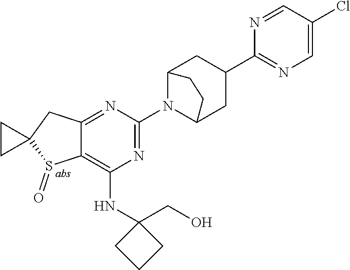 [SMILES: O=[S@]1c2c(nc(N3C4CCC3CC(c3ncc(Cl)cn3)C4)nc2NC2(CO)CCC2)CC12CC2]

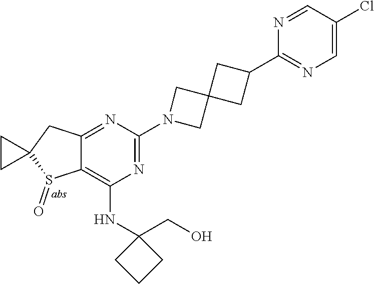 [SMILES: O=[S@]1c2c(nc(N3CC4(CC(c5ncc(Cl)cn5)C4)C3)nc2NC2(CO)CCC2)CC12CC2]

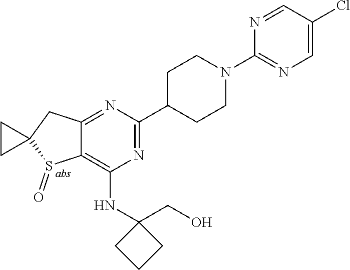 [SMILES: O=[S@]1c2c(nc(C3CCN(c4ncc(Cl)cn4)CC3)nc2NC2(CO)CCC2)CC12CC2]

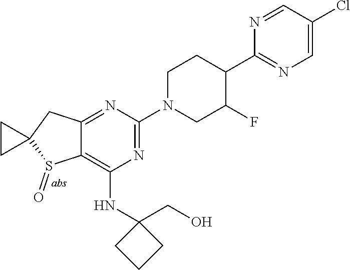 [SMILES: O=[S@]1c2c(nc(N3CCC(c4ncc(Cl)cn4)C(F)C3)nc2NC2(CO)CCC2)CC12CC2]

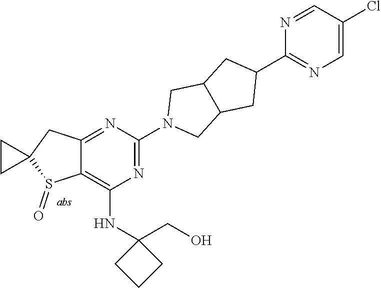 [SMILES: O=[S@]1c2c(nc(N3CC4CC(c5ncc(Cl)cn5)CC4C3)nc2NC2(CO)CCC2)CC12CC2]

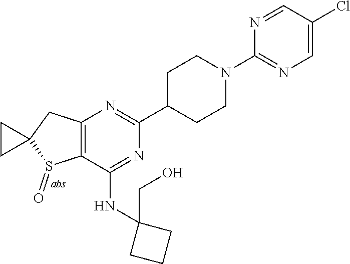 [SMILES: O=[S@]1c2c(nc(C3CCN(c4ncc(Cl)cn4)CC3)nc2NC2(CO)CCC2)CC12CC2]

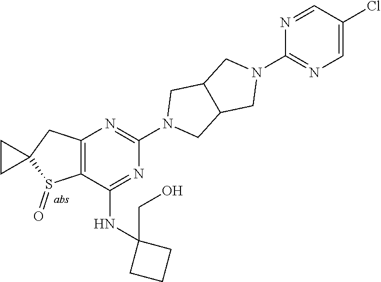 [SMILES: O=[S@]1c2c(nc(N3CC4CN(c5ncc(Cl)cn5)CC4C3)nc2NC2(CO)CCC2)CC12CC2]

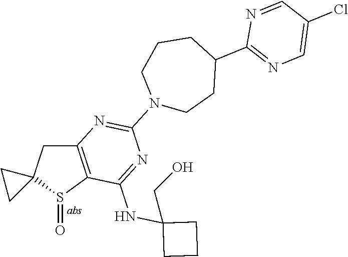 [SMILES: O=[S@]1c2c(nc(N3CCCC(c4ncc(Cl)cn4)CC3)nc2NC2(CO)CCC2)CC12CC2]

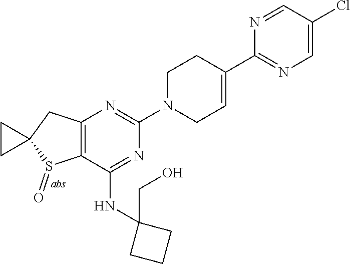

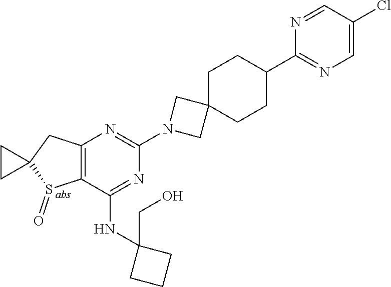 [SMILES: O=[S@]1c2c(nc(N3CC4(CCC(c5ncc(Cl)cn5)CC4)C3)nc2NC2(CO)CCC2)CC12CC2]

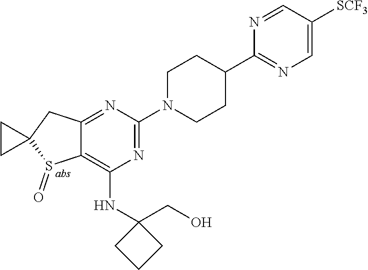

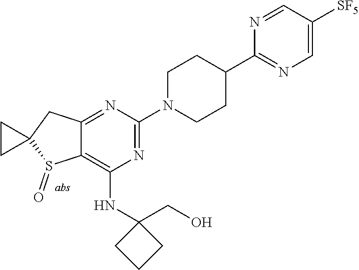

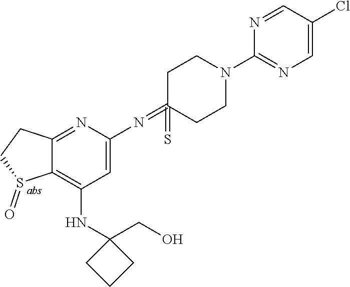

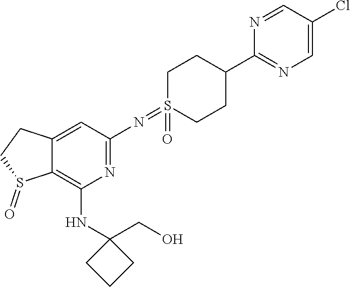 [SMILES: O=[S@@]1CCc2cc(N=S3(=O)CCC(c4ncc(Cl)cn4)CC3)nc(NC3(CO)CCC3)c21]

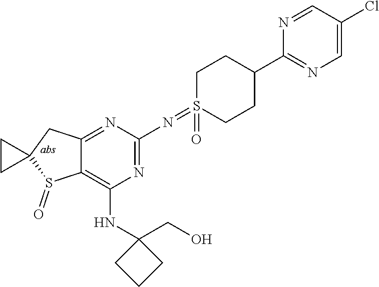 [SMILES: O=[S@]1c2c(nc(N=S3(=O)CCC(c4ncc(Cl)cn4)CC3)nc2NC2(CO)CCC2)CC12CC2]

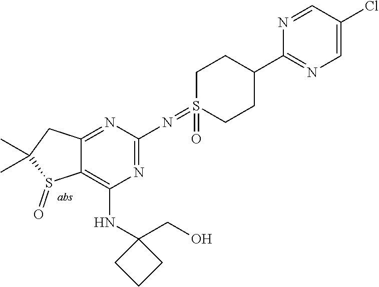 [SMILES: CC1(C)Cc2nc(N=S3(=O)CCC(c4ncc(Cl)cn4)CC3)nc(NC3(CO)CCC3)c2[S@@]1=O]

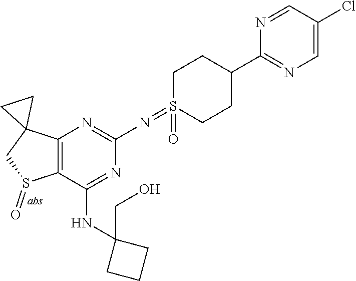 [SMILES: O=[S@@]1CC2(CC2)c2nc(N=S3(=O)CCC(c4ncc(Cl)cn4)CC3)nc(NC3(CO)CCC3)c21]

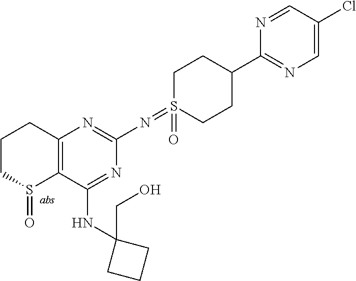 [SMILES: O=[S@@]1CCCc2nc(N=S3(=O)CCC(c4ncc(Cl)cn4)CC3)nc(NC3(CO)CCC3)c21]

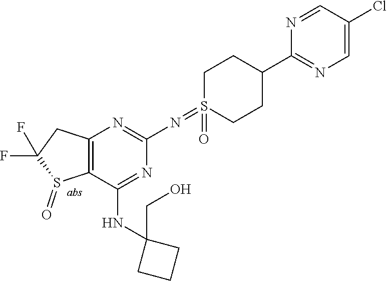 [SMILES: O=[S@]1c2c(nc(N=S3(=O)CCC(c4ncc(Cl)cn4)CC3)nc2NC2(CO)CCC2)CC1(F)F]

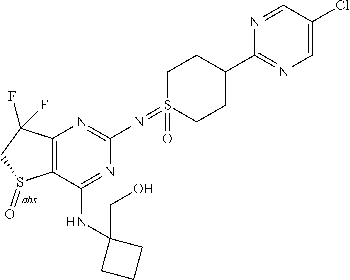 [SMILES: O=[S@@]1CC(F)(F)c2nc(N=S3(=O)CCC(c4ncc(Cl)cn4)CC3)nc(NC3(CO)CCC3)c21]

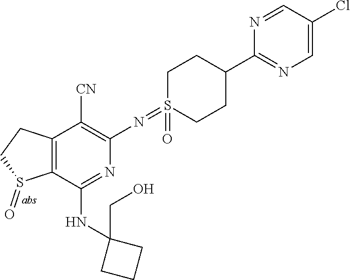 [SMILES: N#Cc1c(N=S2(=O)CCC(c3ncc(Cl)cn3)CC2)nc(NC2(CO)CCC2)c2c1CC[S@]2=O]

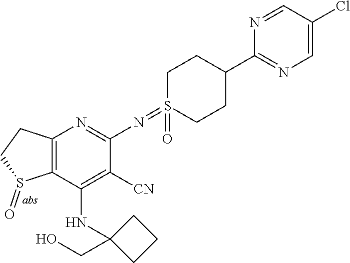 [SMILES: N#Cc1c(N=S2(=O)CCC(c3ncc(Cl)cn3)CC2)nc2c(c1NC1(CO)CCC1)[S@](=O)CC2]

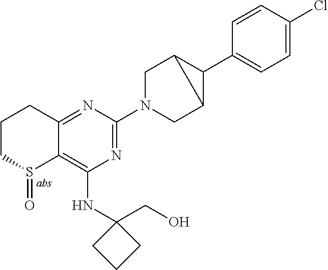 [SMILES: O=[S@@]1CCCc2nc(N3CC4C(C3)C4c3ccc(Cl)cc3)nc(NC3(CO)CCC3)c21]

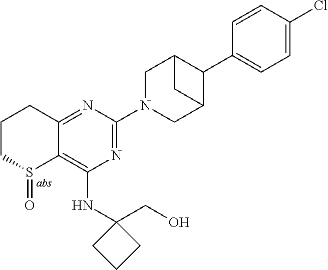 [SMILES: O=[S@@]1CCCc2nc(N3CC4CC(C3)C4c3ccc(Cl)cc3)nc(NC3(CO)CCC3)c21]

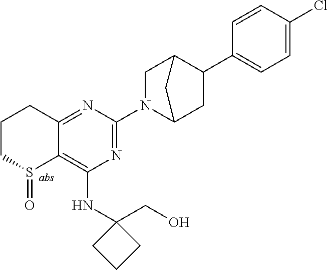 [SMILES: O=[S@@]1CCCc2nc(N3CC4CC3CC4c3ccc(Cl)cc3)nc(NC3(CO)CCC3)c21]

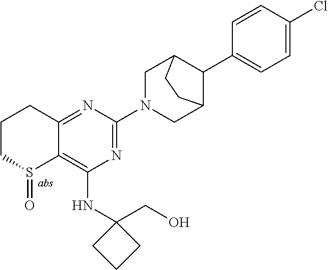 [SMILES: O=[S@@]1CCCc2nc(N3CC4CCC(C3)C4c3ccc(Cl)cc3)nc(NC3(CO)CCC3)c21]

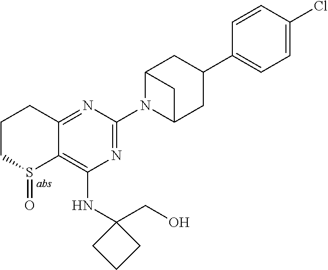 [SMILES: O=[S@@]1CCCc2nc(N3C4CC(c5ccc(Cl)cc5)CC3C4)nc(NC3(CO)CCC3)c21]

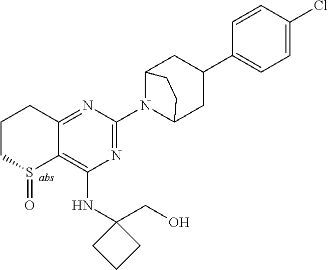 [SMILES: O=[S@@]1CCCc2nc(N3C4CCC3CC(c3ccc(Cl)cc3)C4)nc(NC3(CO)CCC3)c21]

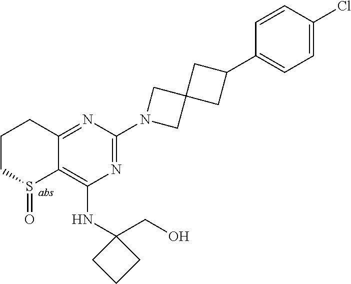 [SMILES: O=[S@@]1CCCc2nc(N3CC4(CC(c5ccc(Cl)cc5)C4)C3)nc(NC3(CO)CCC3)c21]

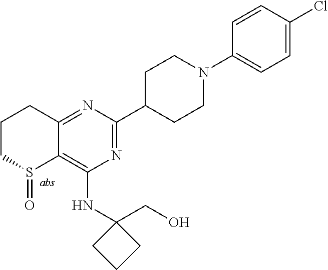 [SMILES: O=[S@@]1CCCc2nc(C3CCN(c4ccc(Cl)cc4)CC3)nc(NC3(CO)CCC3)c21]

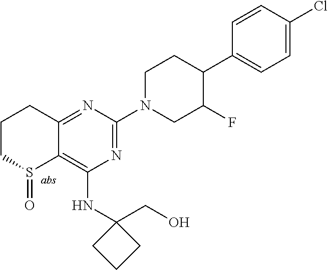 [SMILES: O=[S@@]1CCCc2nc(N3CCC(c4ccc(Cl)cc4)C(F)C3)nc(NC3(CO)CCC3)c21]

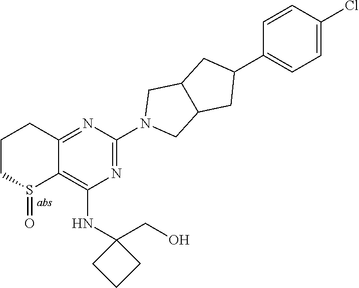 [SMILES: O=[S@@]1CCCc2nc(N3CC4CC(c5ccc(Cl)cc5)CC4C3)nc(NC3(CO)CCC3)c21]

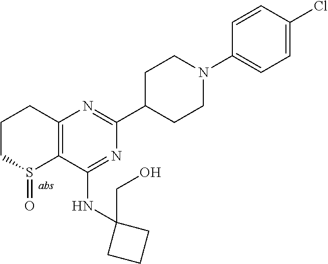 [SMILES: O=[S@@]1CCCc2nc(C3CCN(c4ccc(Cl)cc4)CC3)nc(NC3(CO)CCC3)c21]

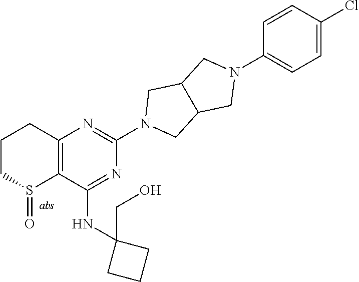 [SMILES: O=[S@@]1CCCc2nc(N3CC4CN(c5ccc(Cl)cc5)CC4C3)nc(NC3(CO)CCC3)c21]

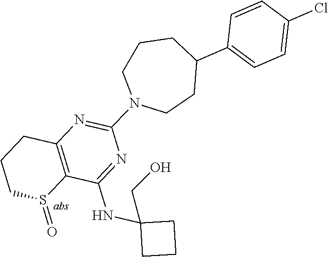 [SMILES: O=[S@@]1CCCc2nc(N3CCCC(c4ccc(Cl)cc4)CC3)nc(NC3(CO)CCC3)c21]

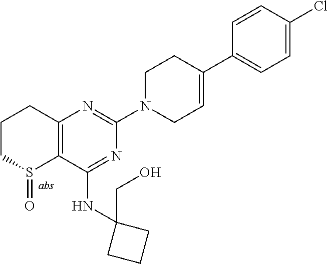 [SMILES: O=[S@@]1CCCc2nc(N3CC=C(c4ccc(Cl)cc4)CC3)nc(NC3(CO)CCC3)c21]

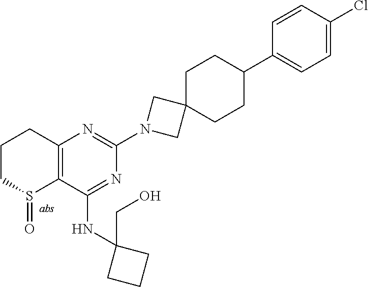 [SMILES: O=[S@@]1CCCc2nc(N3CC4(CCC(c5ccc(Cl)cc5)CC4)C3)nc(NC3(CO)CCC3)c21]

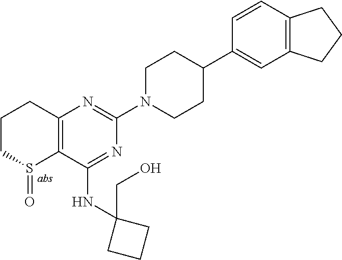 [SMILES: O=[S@@]1CCCc2nc(N3CCC(c4ccc5c(c4)CCC5)CC3)nc(NC3(CO)CCC3)c21]

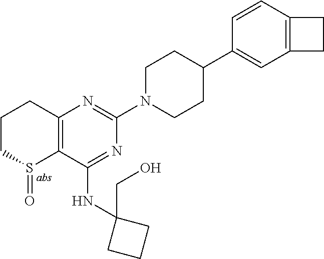 [SMILES: O=[S@@]1CCCc2nc(N3CCC(c4ccc5c(c4)CC5)CC3)nc(NC3(CO)CCC3)c21]

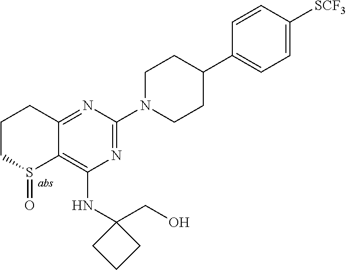

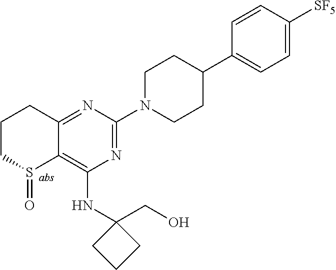

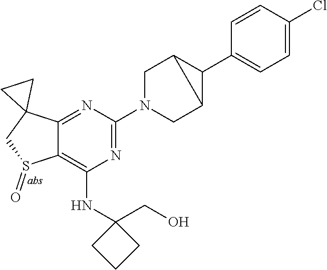 [SMILES: O=[S@@]1CC2(CC2)c2nc(N3CC4C(C3)C4c3ccc(Cl)cc3)nc(NC3(CO)CCC3)c21]

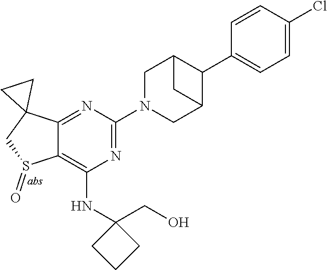 [SMILES: O=[S@@]1CC2(CC2)c2nc(N3CC4CC(C3)C4c3ccc(Cl)cc3)nc(NC3(CO)CCC3)c21]

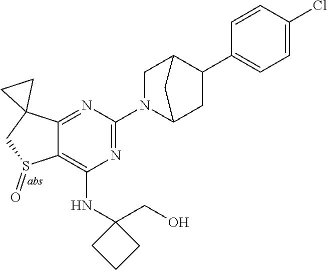 [SMILES: O=[S@@]1CC2(CC2)c2nc(N3CC4CC3CC4c3ccc(Cl)cc3)nc(NC3(CO)CCC3)c21]

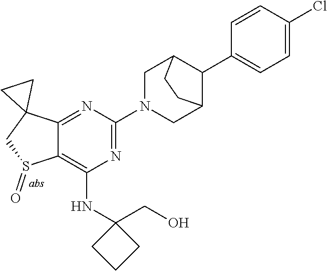 [SMILES: O=[S@@]1CC2(CC2)c2nc(N3CC4CCC(C3)C4c3ccc(Cl)cc3)nc(NC3(CO)CCC3)c21]

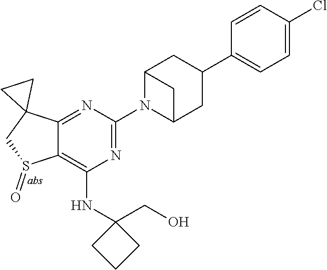 [SMILES: O=[S@@]1CC2(CC2)c2nc(N3C4CC(c5ccc(Cl)cc5)CC3C4)nc(NC3(CO)CCC3)c21]

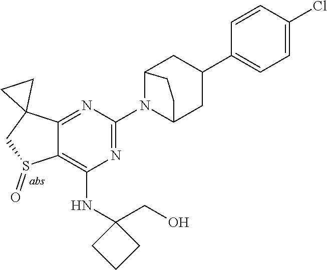 [SMILES: O=[S@@]1CC2(CC2)c2nc(N3C4CCC3CC(c3ccc(Cl)cc3)C4)nc(NC3(CO)CCC3)c21]

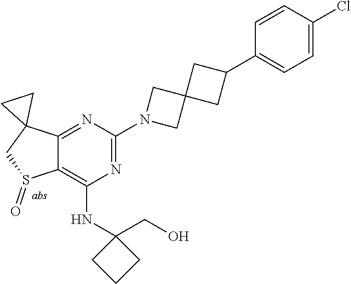 [SMILES: O=[S@@]1CC2(CC2)c2nc(N3CC4(CC(c5ccc(Cl)cc5)C4)C3)nc(NC3(CO)CCC3)c21]

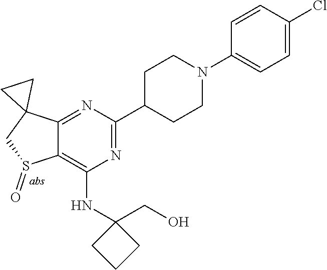 [SMILES: O=[S@@]1CC2(CC2)c2nc(C3CCN(c4ccc(Cl)cc4)CC3)nc(NC3(CO)CCC3)c21]

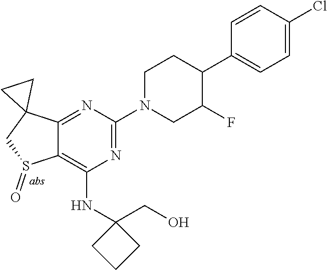 [SMILES: O=[S@@]1CC2(CC2)c2nc(N3CCC(c4ccc(Cl)cc4)C(F)C3)nc(NC3(CO)CCC3)c21]

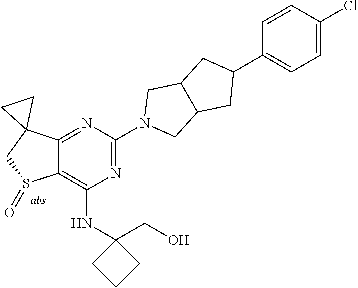 [SMILES: O=[S@@]1CC2(CC2)c2nc(N3CC4CC(c5ccc(Cl)cc5)CC4C3)nc(NC3(CO)CCC3)c21]

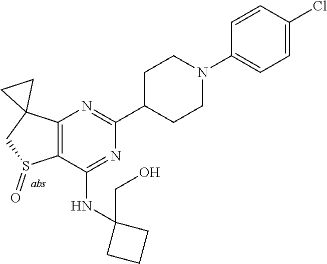 [SMILES: O=[S@@]1CC2(CC2)c2nc(C3CCN(c4ccc(Cl)cc4)CC3)nc(NC3(CO)CCC3)c21]

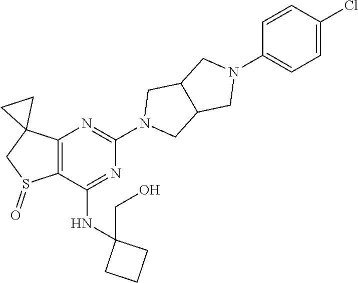 [SMILES: O=S1CC2(CC2)c2nc(N3CC4CN(c5ccc(Cl)cc5)CC4C3)nc(NC3(CO)CCC3)c21]

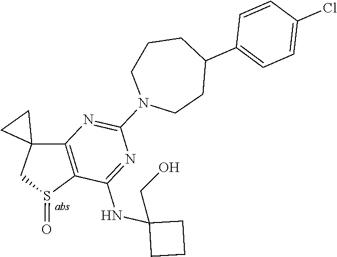 [SMILES: O=[S@@]1CC2(CC2)c2nc(N3CCCC(c4ccc(Cl)cc4)CC3)nc(NC3(CO)CCC3)c21]

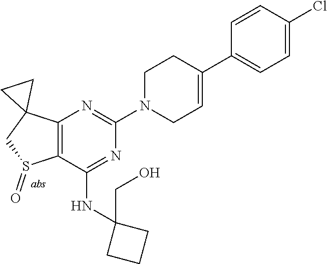 [SMILES: O=[S@@]1CC2(CC2)c2nc(N3CC=C(c4ccc(Cl)cc4)CC3)nc(NC3(CO)CCC3)c21]

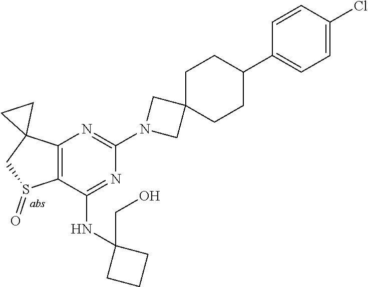 [SMILES: O=[S@@]1CC2(CC2)c2nc(N3CC4(CCC(c5ccc(Cl)cc5)CC4)C3)nc(NC3(CO)CCC3)c21]

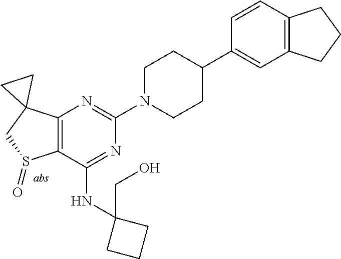 [SMILES: O=[S@@]1CC2(CC2)c2nc(N3CCC(c4ccc5c(c4)CCC5)CC3)nc(NC3(CO)CCC3)c21]

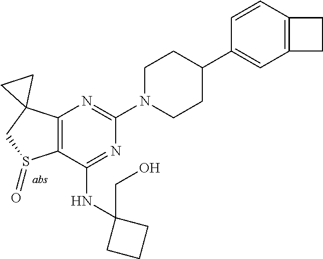 [SMILES: O=[S@@]1CC2(CC2)c2nc(N3CCC(c4ccc5c(c4)CC5)CC3)nc(NC3(CO)CCC3)c21]

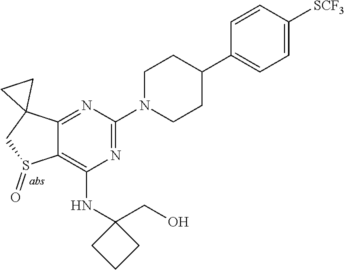

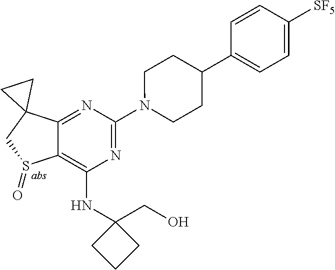

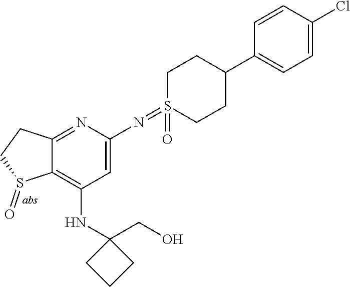 [SMILES: O=[S@@]1CCc2nc(N=S3(=O)CCC(c4ccc(Cl)cc4)CC3)cc(NC3(CO)CCC3)c21]

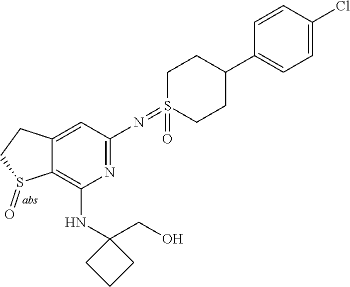 [SMILES: O=[S@@]1CCc2cc(N=S3(=O)CCC(c4ccc(Cl)cc4)CC3)nc(NC3(CO)CCC3)c21]

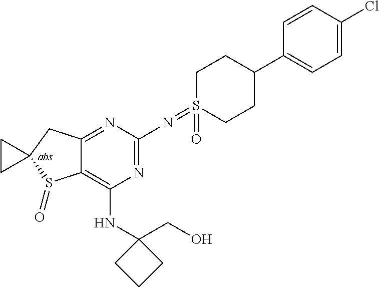

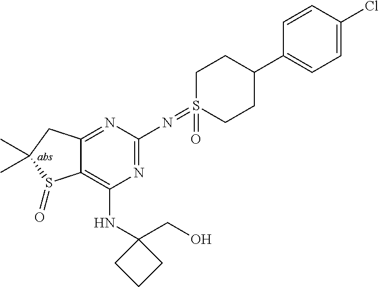

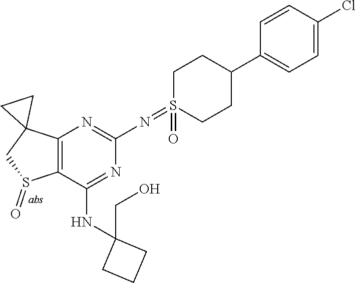 [SMILES: O=[S@@]1CC2(CC2)c2nc(N=S3(=O)CCC(c4ccc(Cl)cc4)CC3)nc(NC3(CO)CCC3)c21]

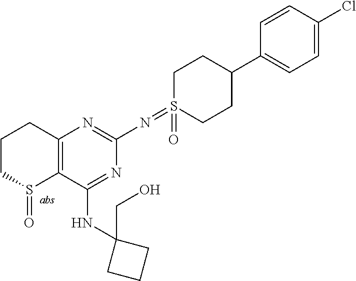 [SMILES: O=[S@@]1CCCc2nc(N=S3(=O)CCC(c4ccc(Cl)cc4)CC3)nc(NC3(CO)CCC3)c21]

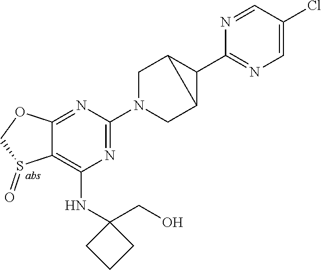 [SMILES: O=[S@@]1COc2nc(N3CC4C(C3)C4c3ncc(Cl)cn3)nc(NC3(CO)CCC3)c21]

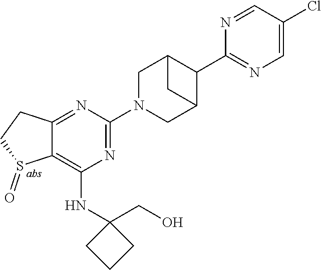 [SMILES: O=[S@@]1CCc2nc(N3CC4CC(C3)C4c3ncc(Cl)cn3)nc(NC3(CO)CCC3)c21]

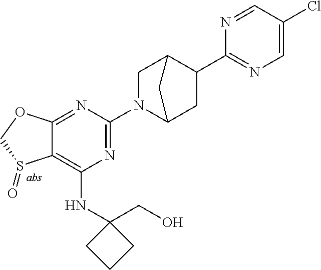 [SMILES: O=[S@@]1COc2nc(N3CC4CC3CC4c3ncc(Cl)cn3)nc(NC3(CO)CCC3)c21]

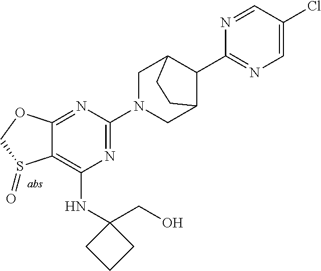 [SMILES: O=[S@@]1COc2nc(N3CC4CCC(C3)C4c3ncc(Cl)cn3)nc(NC3(CO)CCC3)c21]

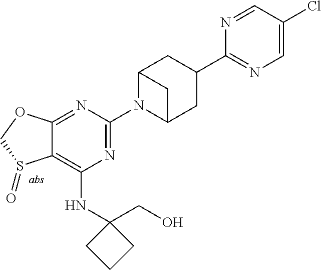 [SMILES: O=[S@@]1COc2nc(N3C4CC(c5ncc(Cl)cn5)CC3C4)nc(NC3(CO)CCC3)c21]

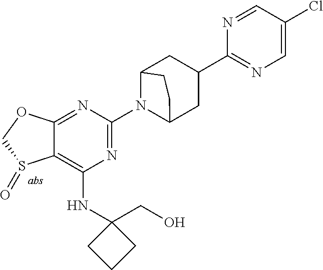 [SMILES: O=[S@@]1COc2nc(N3C4CCC3CC(c3ncc(Cl)cn3)C4)nc(NC3(CO)CCC3)c21]

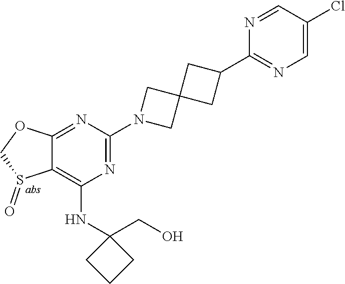 [SMILES: O=[S@@]1COc2nc(N3CC4(CC(c5ncc(Cl)cn5)C4)C3)nc(NC3(CO)CCC3)c21]

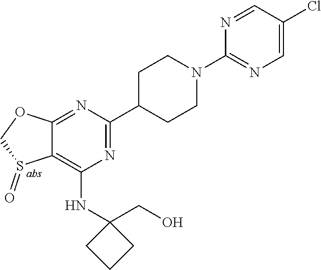 [SMILES: O=[S@@]1COc2nc(C3CCN(c4ncc(Cl)cn4)CC3)nc(NC3(CO)CCC3)c21]

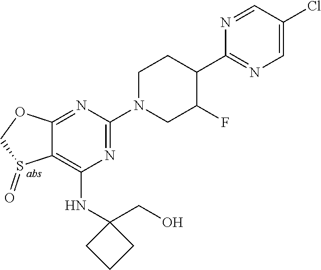 [SMILES: O=[S@@]1COc2nc(N3CCC(c4ncc(Cl)cn4)C(F)C3)nc(NC3(CO)CCC3)c21]

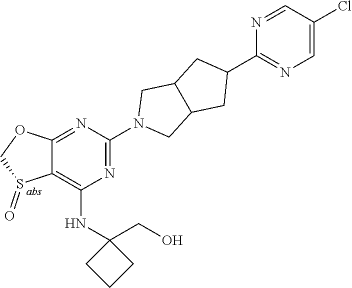 [SMILES: O=[S@@]1COc2nc(N3CC4CC(c5ncc(Cl)cn5)CC4C3)nc(NC3(CO)CCC3)c21]

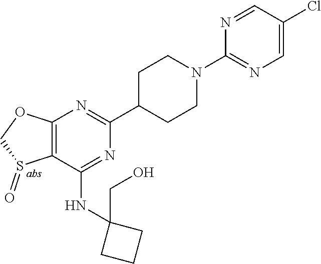 [SMILES: O=[S@@]1COc2nc(C3CCN(c4ncc(Cl)cn4)CC3)nc(NC3(CO)CCC3)c21]

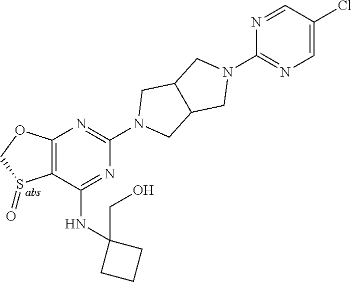 [SMILES: O=[S@@]1COc2nc(N3CC4CN(c5ncc(Cl)cn5)CC4C3)nc(NC3(CO)CCC3)c21]

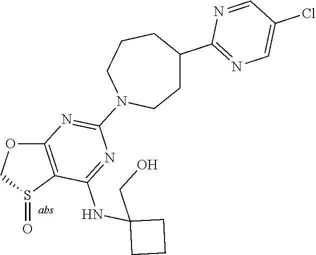 [SMILES: O=[S@@]1COc2nc(N3CCCC(c4ncc(Cl)cn4)CC3)nc(NC3(CO)CCC3)c21]

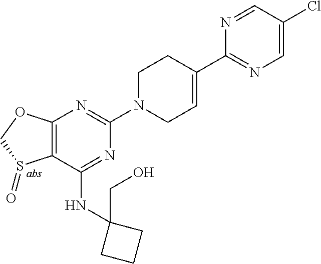 [SMILES: O=[S@@]1COc2nc(N3CC=C(c4ncc(Cl)cn4)CC3)nc(NC3(CO)CCC3)c21]

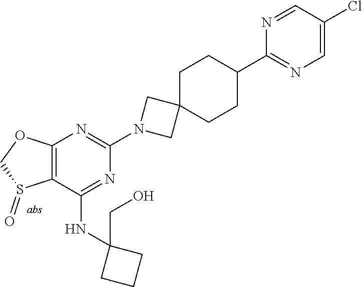 [SMILES: O=[S@@]1COc2nc(N3CC4(CCC(c5ncc(Cl)cn5)CC4)C3)nc(NC3(CO)CCC3)c21]

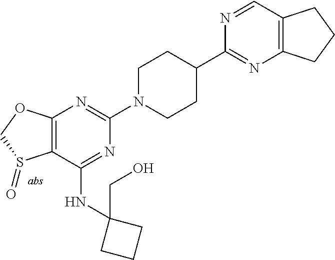 [SMILES: O=[S@@]1COc2nc(N3CCC(c4ncc5c(n4)CCC5)CC3)nc(NC3(CO)CCC3)c21]

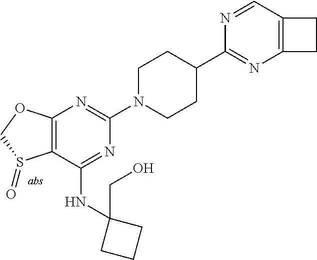 [SMILES: O=[S@@]1COc2nc(N3CCC(c4ncc5c(n4)CC5)CC3)nc(NC3(CO)CCC3)c21]

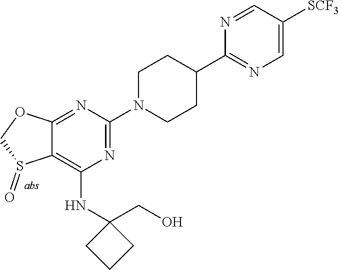

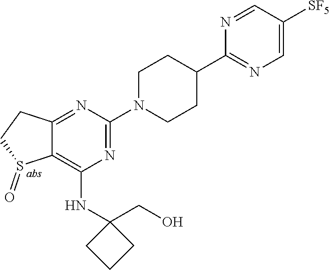

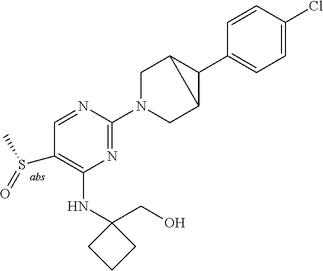 [SMILES: C[S@@](=O)c1cnc(N2CC3C(C2)C3c2ccc(Cl)cc2)nc1NC1(CO)CCC1]

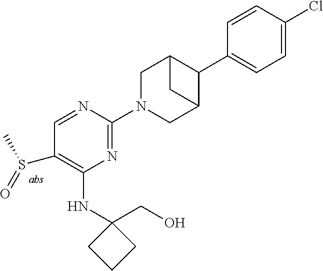 [SMILES: C[S@@](=O)c1cnc(N2CC3CC(C2)C3c2ccc(Cl)cc2)nc1NC1(CO)CCC1]

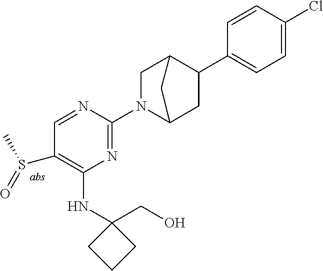 [SMILES: C[S@@](=O)c1cnc(N2CC3CC2CC3c2ccc(Cl)cc2)nc1NC1(CO)CCC1]

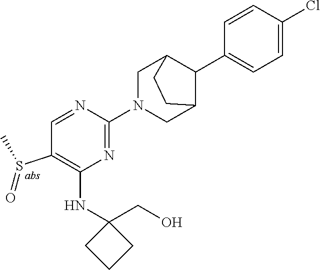 [SMILES: C[S@@](=O)c1cnc(N2CC3CCC(C2)C3c2ccc(Cl)cc2)nc1NC1(CO)CCC1]

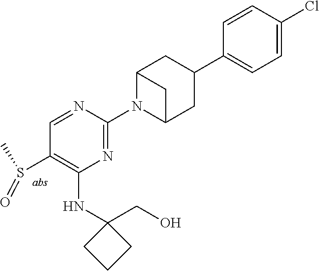 [SMILES: C[S@@](=O)c1cnc(N2C3CC(c4ccc(Cl)cc4)CC2C3)nc1NC1(CO)CCC1]

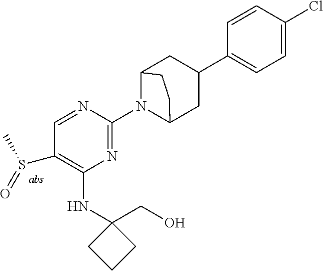 [SMILES: C[S@@](=O)c1cnc(N2C3CCC2CC(c2ccc(Cl)cc2)C3)nc1NC1(CO)CCC1]

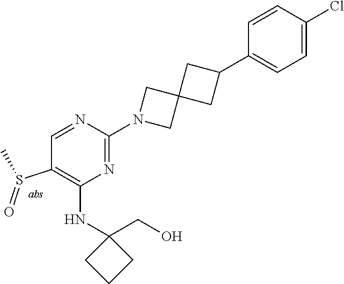 [SMILES: C[S@@](=O)c1cnc(N2CC3(CC(c4ccc(Cl)cc4)C3)C2)nc1NC1(CO)CCC1]

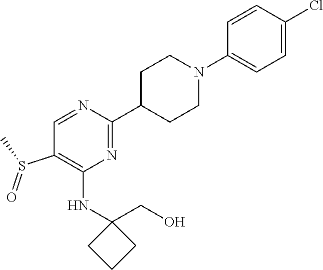 [SMILES: C[S@@](=O)c1cnc(C2CCN(c3ccc(Cl)cc3)CC2)nc1NC1(CO)CCC1]

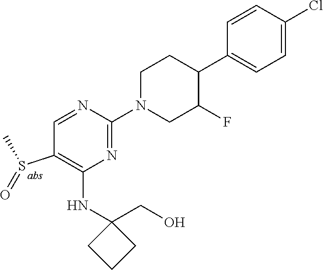 [SMILES: C[S@@](=O)c1cnc(N2CCC(c3ccc(Cl)cc3)C(F)C2)nc1NC1(CO)CCC1]

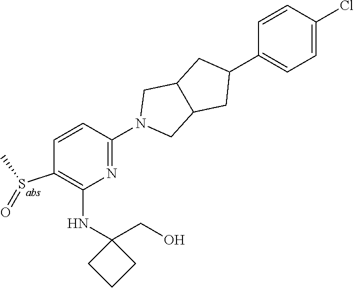 [SMILES: C[S@@](=O)c1ccc(N2CC3CC(c4ccc(Cl)cc4)CC3C2)nc1NC1(CO)CCC1]

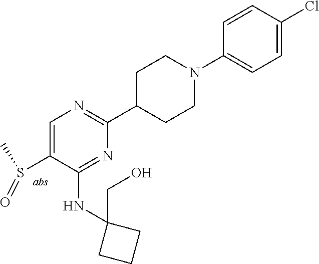 [SMILES: C[S@@](=O)c1cnc(C2CCN(c3ccc(Cl)cc3)CC2)nc1NC1(CO)CCC1]

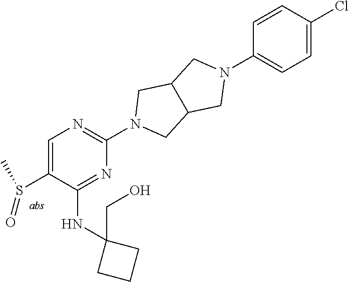 [SMILES: C[S@@](=O)c1cnc(N2CC3CN(c4ccc(Cl)cc4)CC3C2)nc1NC1(CO)CCC1]

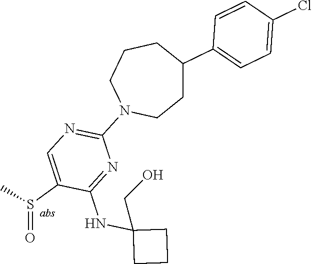 [SMILES: C[S@@](=O)c1cnc(N2CCCC(c3ccc(Cl)cc3)CC2)nc1NC1(CO)CCC1]

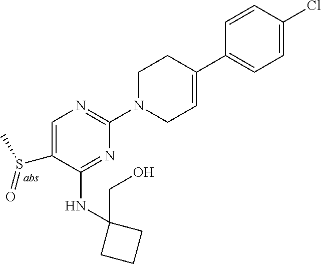 [SMILES: C[S@@](=O)c1cnc(N2CC=C(c3ccc(Cl)cc3)CC2)nc1NC1(CO)CCC1]

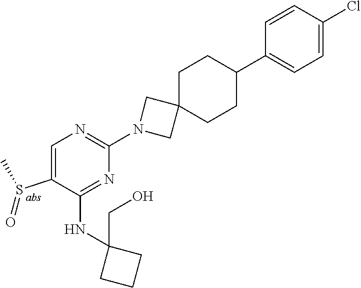 [SMILES: C[S@@](=O)c1cnc(N2CC3(CCC(c4ccc(Cl)cc4)CC3)C2)nc1NC1(CO)CCC1]

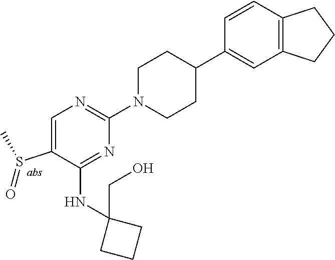 [SMILES: C[S@@](=O)c1cnc(N2CCC(c3ccc4c(c3)CCC4)CC2)nc1NC1(CO)CCC1]

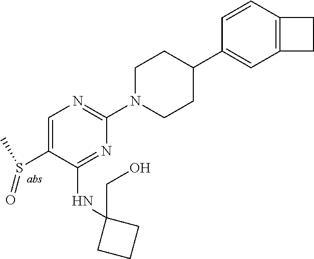 [SMILES: C[S@@](=O)c1cnc(N2CCC(c3ccc4c(c3)CC4)CC2)nc1NC1(CO)CCC1]

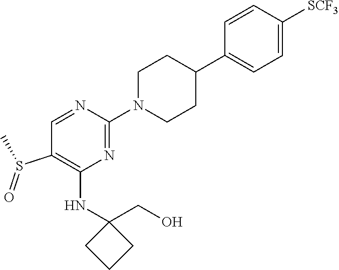

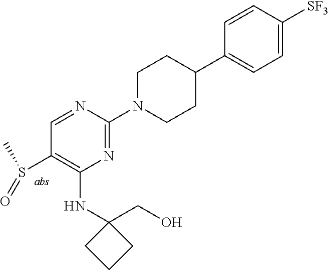

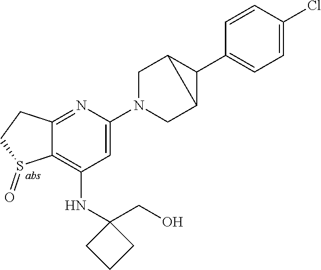 [SMILES: O=[S@@]1CCc2nc(N3CC4C(C3)C4c3ccc(Cl)cc3)cc(NC3(CO)CCC3)c21]

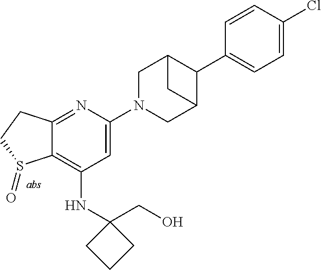 [SMILES: O=[S@@]1CCc2nc(N3CC4CC(C3)C4c3ccc(Cl)cc3)cc(NC3(CO)CCC3)c21]

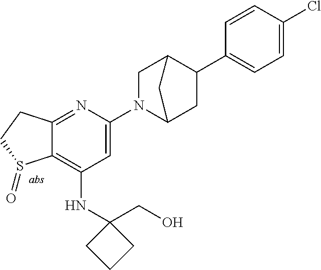 [SMILES: O=[S@@]1CCc2nc(N3CC4CC3CC4c3ccc(Cl)cc3)cc(NC3(CO)CCC3)c21]

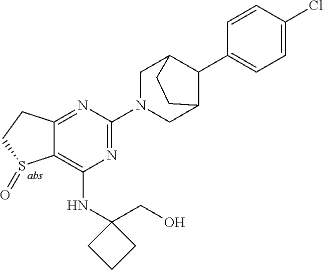 [SMILES: O=[S@@]1CCc2nc(N3CC4CCC(C3)C4c3ccc(Cl)cc3)nc(NC3(CO)CCC3)c21]

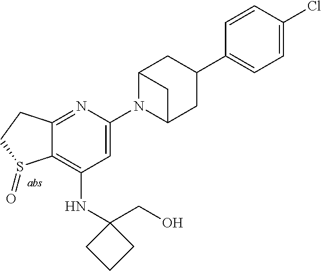 [SMILES: O=[S@@]1CCc2nc(N3C4CC(c5ccc(Cl)cc5)CC3C4)cc(NC3(CO)CCC3)c21]

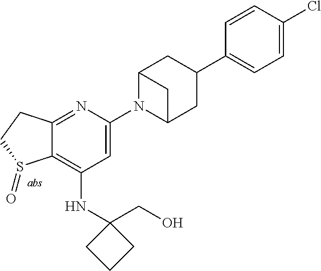 [SMILES: O=[S@@]1CCc2nc(N3C4CC(c5ccc(Cl)cc5)CC3C4)cc(NC3(CO)CCC3)c21]

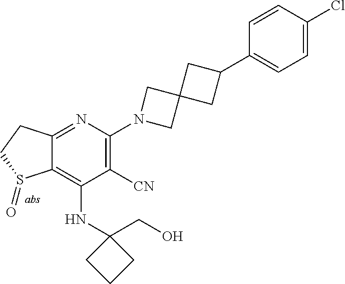 [SMILES: N#Cc1c(N2CC3(CC(c4ccc(Cl)cc4)C3)C2)nc2c(c1NC1(CO)CCC1)[S@](=O)CC2]

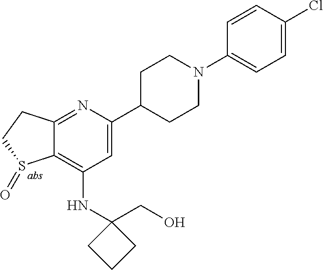 [SMILES: O=[S@@]1CCc2nc(C3CCN(c4ccc(Cl)cc4)CC3)cc(NC3(CO)CCC3)c21]

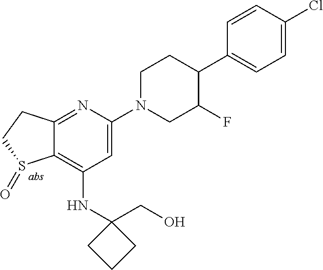 [SMILES: O=[S@@]1CCc2nc(N3CCC(c4ccc(Cl)cc4)C(F)C3)cc(NC3(CO)CCC3)c21]

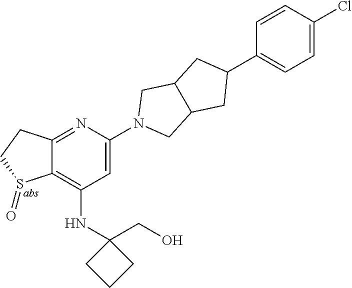 [SMILES: O=[S@@]1CCc2nc(N3CC4CC(c5ccc(Cl)cc5)CC4C3)cc(NC3(CO)CCC3)c21]

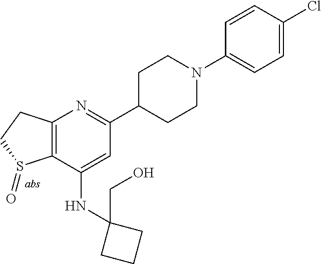 [SMILES: O=[S@@]1CCc2nc(C3CCN(c4ccc(Cl)cc4)CC3)cc(NC3(CO)CCC3)c21]

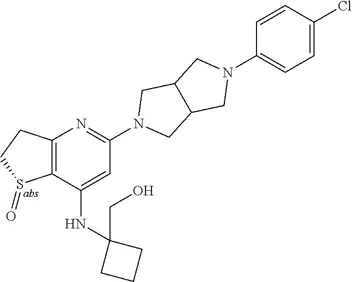 [SMILES: O=[S@@]1CCc2nc(N3CC4CN(c5ccc(Cl)cc5)CC4C3)cc(NC3(CO)CCC3)c21]

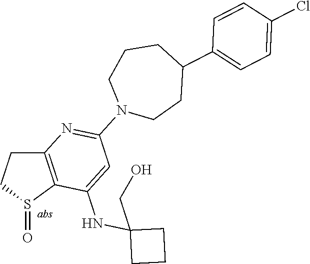 [SMILES: O=[S@@]1CCc2nc(N3CCCC(c4ccc(Cl)cc4)CC3)cc(NC3(CO)CCC3)c21]

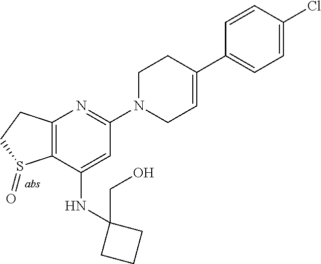 [SMILES: O=[S@@]1CCc2nc(N3CC=C(c4ccc(Cl)cc4)CC3)cc(NC3(CO)CCC3)c21]

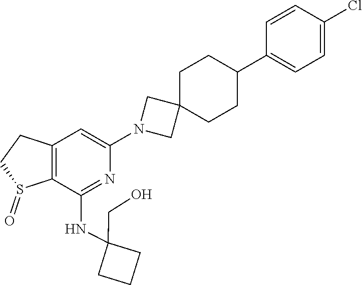 [SMILES: O=[S@@]1CCc2cc(N3CC4(CCC(c5ccc(Cl)cc5)CC4)C3)nc(NC3(CO)CCC3)c21]

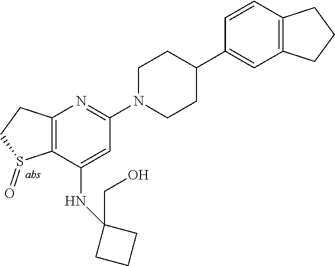 [SMILES: O=[S@@]1CCc2nc(N3CCC(c4ccc5c(c4)CCC5)CC3)cc(NC3(CO)CCC3)c21]

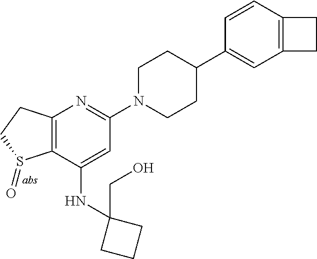 [SMILES: O=[S@@]1CCc2nc(N3CCC(c4ccc5c(c4)CC5)CC3)cc(NC3(CO)CCC3)c21]

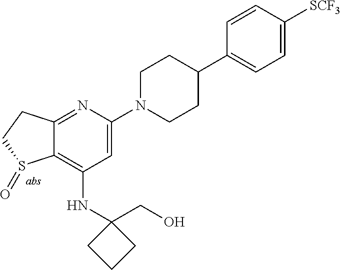

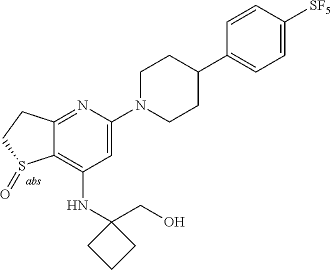

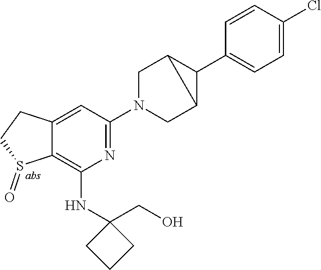 [SMILES: O=[S@@]1CCc2cc(N3CC4C(C3)C4c3ccc(Cl)cc3)nc(NC3(CO)CCC3)c21]

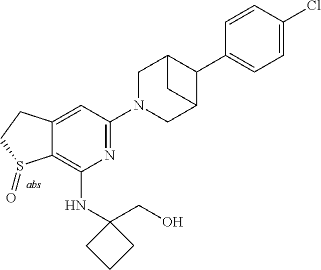 [SMILES: O=[S@@]1CCc2cc(N3CC4CC(C3)C4c3ccc(Cl)cc3)nc(NC3(CO)CCC3)c21]

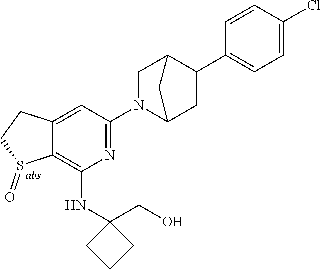 [SMILES: O=[S@@]1CCc2cc(N3CC4CC3CC4c3ccc(Cl)cc3)nc(NC3(CO)CCC3)c21]

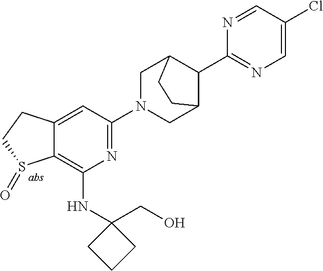 [SMILES: O=[S@@]1CCc2cc(N3CC4CCC(C3)C4c3ncc(Cl)cn3)nc(NC3(CO)CCC3)c21]

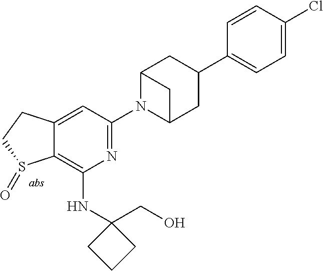 [SMILES: O=[S@@]1CCc2cc(N3C4CC(c5ccc(Cl)cc5)CC3C4)nc(NC3(CO)CCC3)c21]

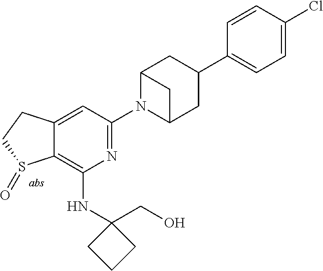 [SMILES: O=[S@@]1CCc2cc(N3C4CC(c5ccc(Cl)cc5)CC3C4)nc(NC3(CO)CCC3)c21]

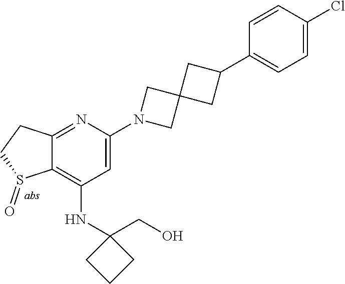 [SMILES: O=[S@@]1CCc2nc(N3CC4(CC(c5ccc(Cl)cc5)C4)C3)cc(NC3(CO)CCC3)c21]

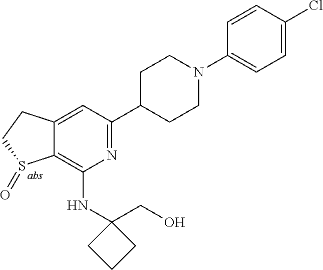 [SMILES: O=[S@@]1CCc2cc(C3CCN(c4ccc(Cl)cc4)CC3)nc(NC3(CO)CCC3)c21]

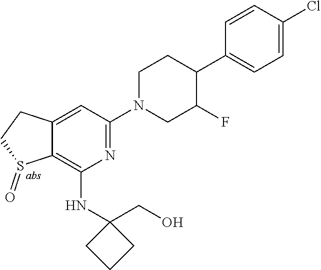 [SMILES: O=[S@@]1CCc2cc(N3CCC(c4ccc(Cl)cc4)C(F)C3)nc(NC3(CO)CCC3)c21]

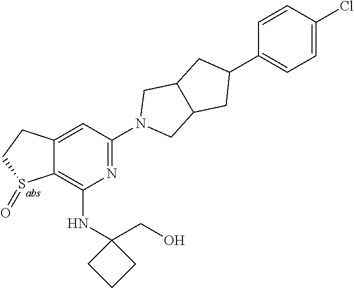 [SMILES: O=[S@@]1CCc2cc(N3CC4CC(c5ccc(Cl)cc5)CC4C3)nc(NC3(CO)CCC3)c21]

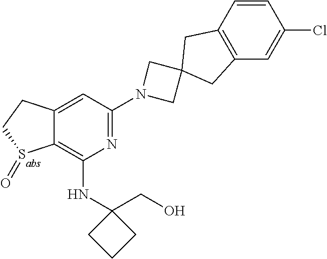 [SMILES: O=[S@@]1CCc2cc(N3CC4(Cc5ccc(Cl)cc5C4)C3)nc(NC3(CO)CCC3)c21]

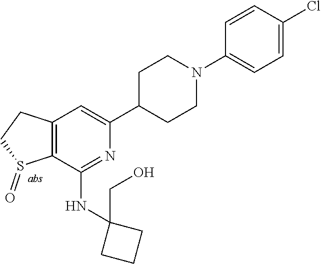 [SMILES: O=[S@@]1CCc2cc(C3CCN(c4ccc(Cl)cc4)CC3)nc(NC3(CO)CCC3)c21]

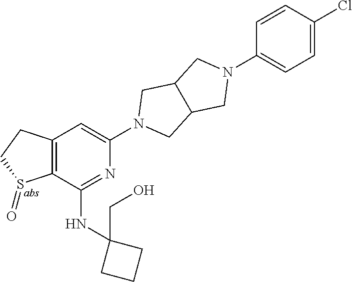 [SMILES: O=[S@@]1CCc2cc(N3CC4CN(c5ccc(Cl)cc5)CC4C3)nc(NC3(CO)CCC3)c21]

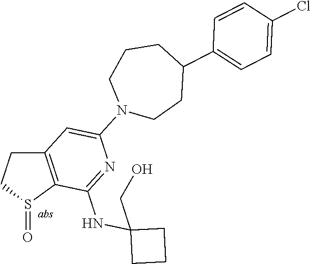 [SMILES: O=[S@@]1CCc2cc(N3CCCC(c4ccc(Cl)cc4)CC3)nc(NC3(CO)CCC3)c21]

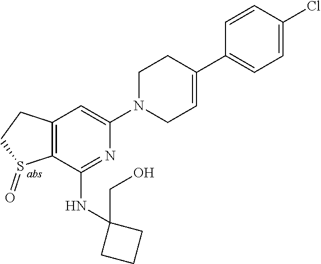 [SMILES: O=[S@@]1CCc2cc(N3CC=C(c4ccc(Cl)cc4)CC3)nc(NC3(CO)CCC3)c21]

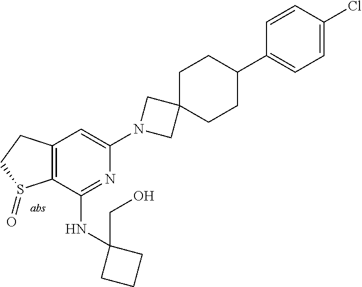 [SMILES: O=[S@@]1CCc2cc(N3CC4(CCC(c5ccc(Cl)cc5)CC4)C3)nc(NC3(CO)CCC3)c21]

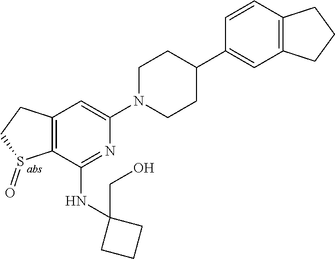 [SMILES: O=[S@@]1CCc2cc(N3CCC(c4ccc5c(c4)CCC5)CC3)nc(NC3(CO)CCC3)c21]

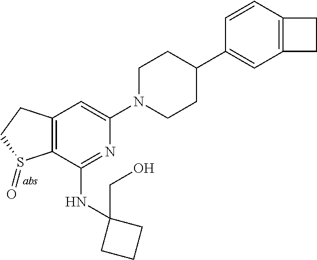 [SMILES: O=[S@@]1CCc2cc(N3CCC(c4ccc5c(c4)CC5)CC3)nc(NC3(CO)CCC3)c21]

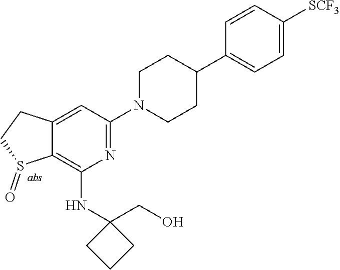

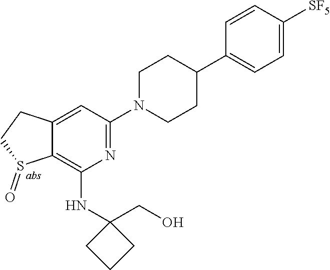

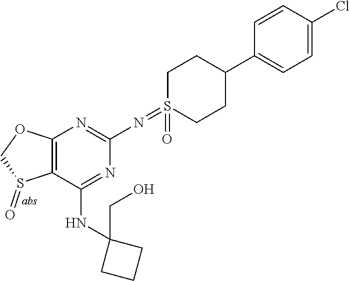 [SMILES: O=[S@@]1COc2nc(N=S3(=O)CCC(c4ccc(Cl)cc4)CC3)nc(NC3(CO)CCC3)c21]

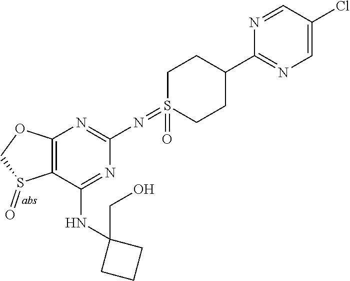 [SMILES: O=[S@@]1COc2nc(N=S3(=O)CCC(c4ncc(Cl)cn4)CC3)nc(NC3(CO)CCC3)c21]

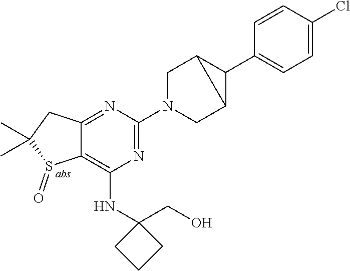 [SMILES: CC1(C)Cc2nc(N3CC4C(C3)C4c3ccc(Cl)cc3)nc(NC3(CO)CCC3)c2[S@@]1=O]

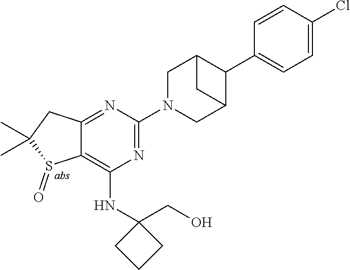 [SMILES: CC1(C)Cc2nc(N3CC4CC(C3)C4c3ccc(Cl)cc3)nc(NC3(CO)CCC3)c2[S@@]1=O]

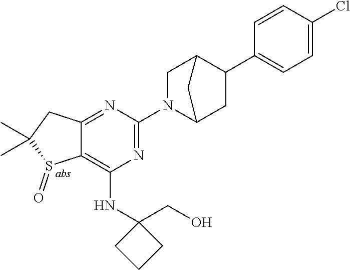 [SMILES: CC1(C)Cc2nc(N3CC4CC3CC4c3ccc(Cl)cc3)nc(NC3(CO)CCC3)c2[S@@]1=O]

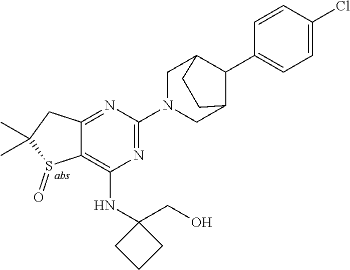 [SMILES: CC1(C)Cc2nc(N3CC4CCC(C3)C4c3ccc(Cl)cc3)nc(NC3(CO)CCC3)c2[S@@]1=O]

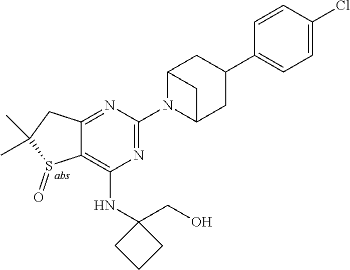 [SMILES: CC1(C)Cc2nc(N3C4CC(c5ccc(Cl)cc5)CC3C4)nc(NC3(CO)CCC3)c2[S@@]1=O]

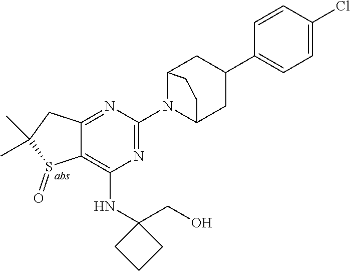 [SMILES: CC1(C)Cc2nc(N3C4CCC3CC(c3ccc(Cl)cc3)C4)nc(NC3(CO)CCC3)c2[S@@]1=O]

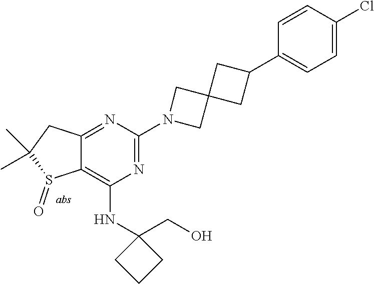 [SMILES: CC1(C)Cc2nc(N3CC4(CC(c5ccc(Cl)cc5)C4)C3)nc(NC3(CO)CCC3)c2[S@@]1=O]

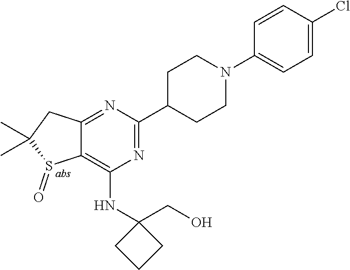 [SMILES: CC1(C)Cc2nc(C3CCN(c4ccc(Cl)cc4)CC3)nc(NC3(CO)CCC3)c2[S@@]1=O]

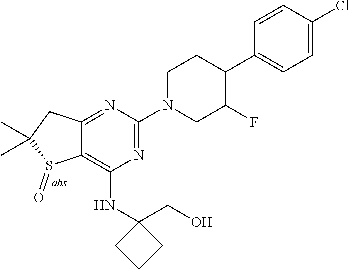 [SMILES: CC1(C)Cc2nc(N3CCC(c4ccc(Cl)cc4)C(F)C3)nc(NC3(CO)CCC3)c2[S@@]1=O]

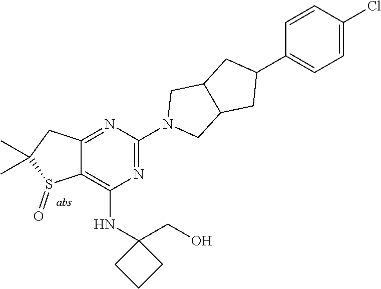 [SMILES: CC1(C)Cc2nc(N3CC4CC(c5ccc(Cl)cc5)CC4C3)nc(NC3(CO)CCC3)c2[S@@]1=O]

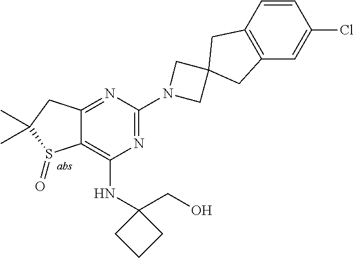 [SMILES: CC1(C)Cc2nc(N3CC4(Cc5ccc(Cl)cc5C4)C3)nc(NC3(CO)CCC3)c2[S@@]1=O]

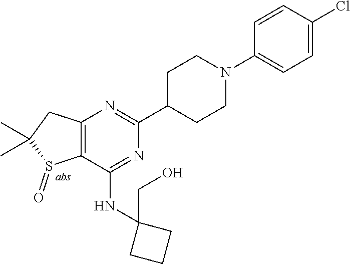 [SMILES: CC1(C)Cc2nc(C3CCN(c4ccc(Cl)cc4)CC3)nc(NC3(CO)CCC3)c2[S@@]1=O]

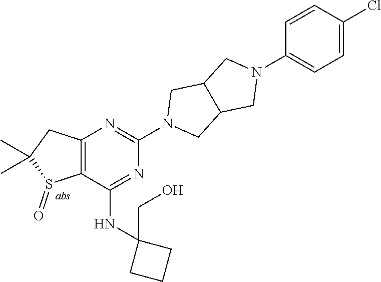 [SMILES: CC1(C)Cc2nc(N3CC4CN(c5ccc(Cl)cc5)CC4C3)nc(NC3(CO)CCC3)c2[S@@]1=O]

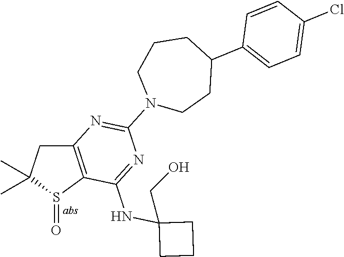 [SMILES: CC1(C)Cc2nc(N3CCCC(c4ccc(Cl)cc4)CC3)nc(NC3(CO)CCC3)c2[S@@]1=O]

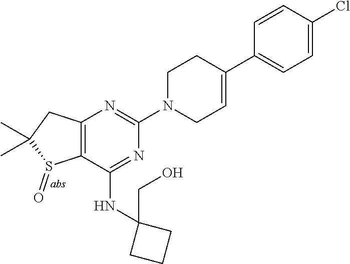 [SMILES: CC1(C)Cc2nc(N3CC=C(c4ccc(Cl)cc4)CC3)nc(NC3(CO)CCC3)c2[S@@]1=O]

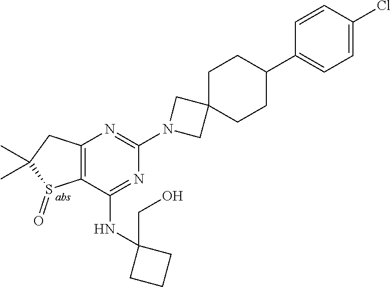 [SMILES: CC1(C)Cc2nc(N3CC4(CCC(c5ccc(Cl)cc5)CC4)C3)nc(NC3(CO)CCC3)c2[S@@]1=O]

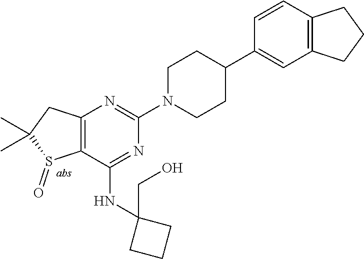 [SMILES: CC1(C)Cc2nc(N3CCC(c4ccc5c(c4)CCC5)CC3)nc(NC3(CO)CCC3)c2[S@@]1=O]

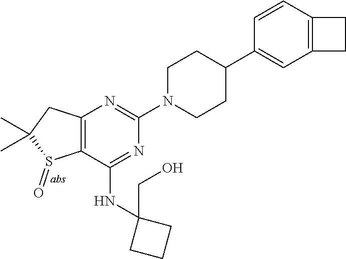 [SMILES: CC1(C)Cc2nc(N3CCC(c4ccc5c(c4)CC5)CC3)nc(NC3(CO)CCC3)c2[S@@]1=O]

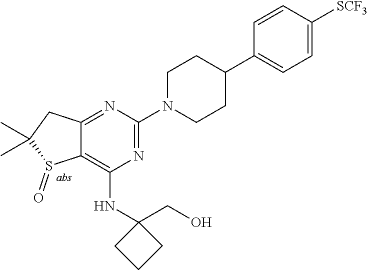

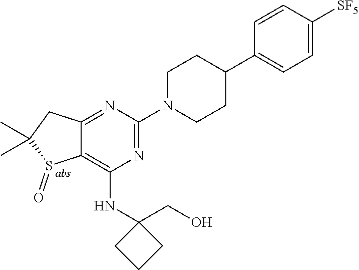

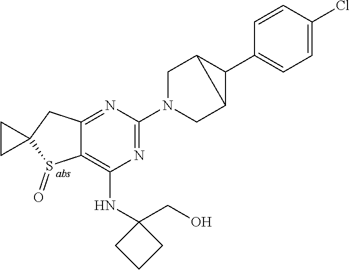 [SMILES: O=[S@]1c2c(nc(N3CC4C(C3)C4c3ccc(Cl)cc3)nc2NC2(CO)CCC2)CC12CC2]

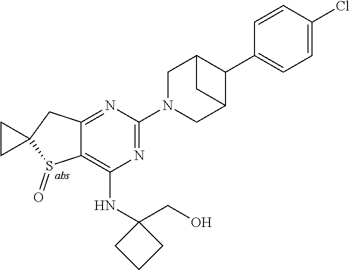 [SMILES: O=[S@]1c2c(nc(N3CC4CC(C3)C4c3ccc(Cl)cc3)nc2NC2(CO)CCC2)CC12CC2]

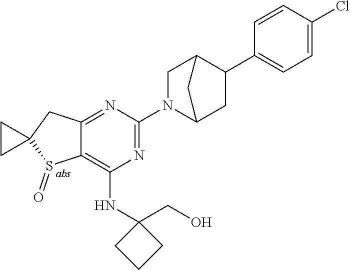 [SMILES: O=[S@]1c2c(nc(N3CC4CC3CC4c3ccc(Cl)cc3)nc2NC2(CO)CCC2)CC12CC2]

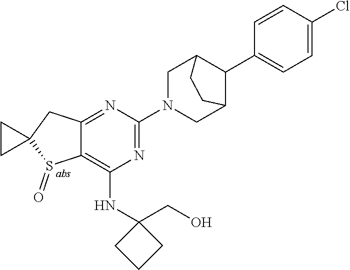 [SMILES: O=[S@]1c2c(nc(N3CC4CCC(C3)C4c3ccc(Cl)cc3)nc2NC2(CO)CCC2)CC12CC2]

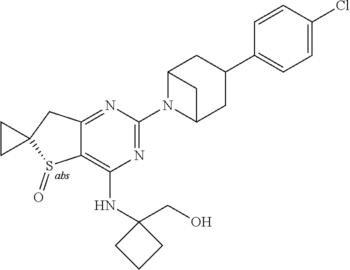 [SMILES: O=[S@]1c2c(nc(N3C4CC(c5ccc(Cl)cc5)CC3C4)nc2NC2(CO)CCC2)CC12CC2]

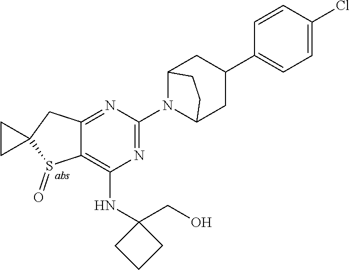 [SMILES: O=[S@]1c2c(nc(N3C4CCC3CC(c3ccc(Cl)cc3)C4)nc2NC2(CO)CCC2)CC12CC2]

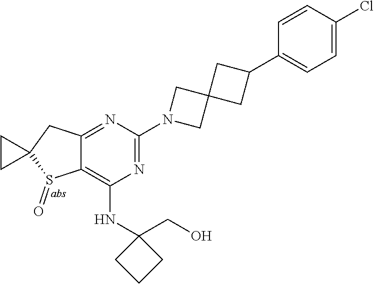 [SMILES: O=[S@]1c2c(nc(N3CC4(CC(c5ccc(Cl)cc5)C4)C3)nc2NC2(CO)CCC2)CC12CC2]

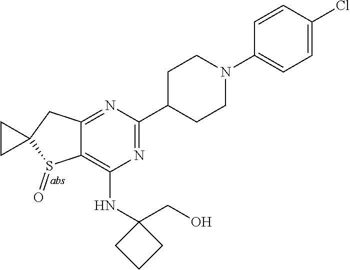 [SMILES: O=[S@]1c2c(nc(C3CCN(c4ccc(Cl)cc4)CC3)nc2NC2(CO)CCC2)CC12CC2]

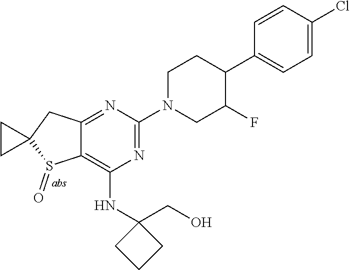 [SMILES: O=[S@]1c2c(nc(N3CCC(c4ccc(Cl)cc4)C(F)C3)nc2NC2(CO)CCC2)CC12CC2]

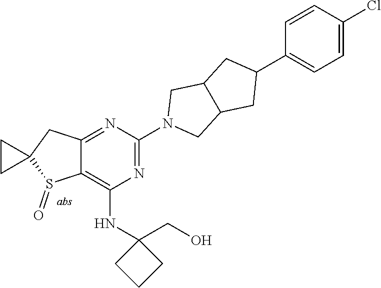 [SMILES: O=[S@]1c2c(nc(N3CC4CC(c5ccc(Cl)cc5)CC4C3)nc2NC2(CO)CCC2)CC12CC2]

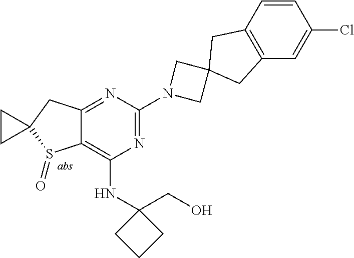 [SMILES: O=[S@]1c2c(nc(N3CC4(Cc5ccc(Cl)cc5C4)C3)nc2NC2(CO)CCC2)CC12CC2]

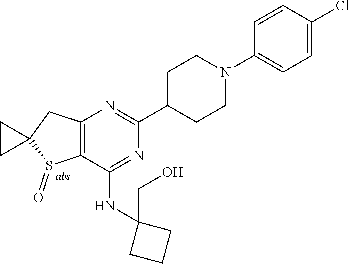 [SMILES: O=[S@]1c2c(nc(C3CCN(c4ccc(Cl)cc4)CC3)nc2NC2(CO)CCC2)CC12CC2]

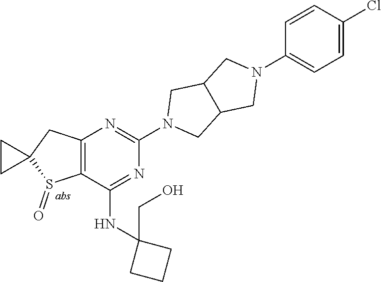 [SMILES: O=[S@]1c2c(nc(N3CC4CN(c5ccc(Cl)cc5)CC4C3)nc2NC2(CO)CCC2)CC12CC2]

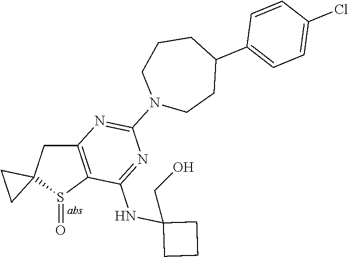 [SMILES: O=[S@]1c2c(nc(N3CCCC(c4ccc(Cl)cc4)CC3)nc2NC2(CO)CCC2)CC12CC2]

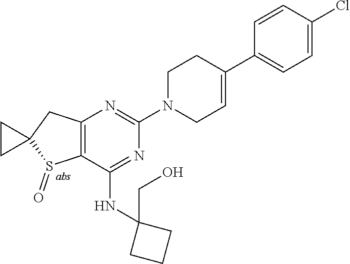 [SMILES: O=[S@]1c2c(nc(N3CC=C(c4ccc(Cl)cc4)CC3)nc2NC2(CO)CCC2)CC12CC2]

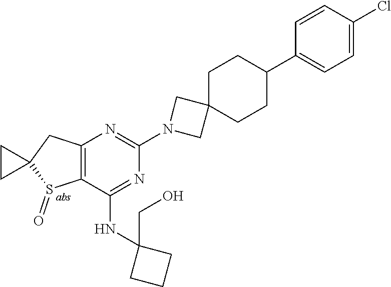 [SMILES: O=[S@]1c2c(nc(N3CC4(CCC(c5ccc(Cl)cc5)CC4)C3)nc2NC2(CO)CCC2)CC12CC2]

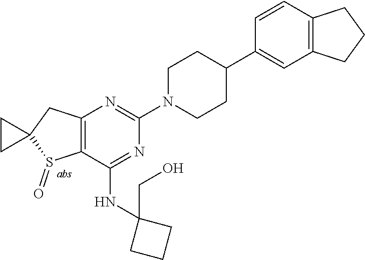 [SMILES: O=[S@]1c2c(nc(N3CCC(c4ccc5c(c4)CCC5)CC3)nc2NC2(CO)CCC2)CC12CC2]

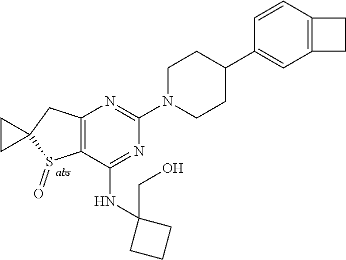 [SMILES: O=[S@]1c2c(nc(N3CCC(c4ccc5c(c4)CC5)CC3)nc2NC2(CO)CCC2)CC12CC2]

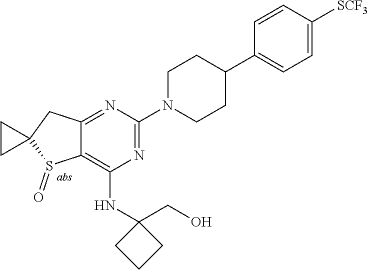

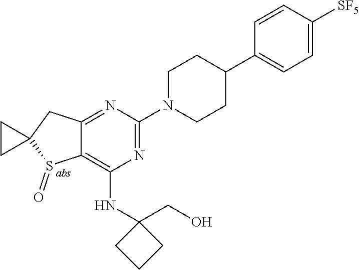

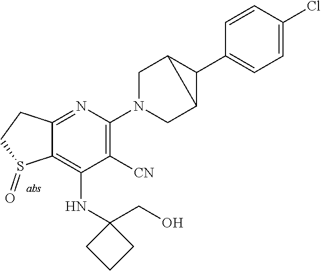 [SMILES: N#Cc1c(N2CC3C(C2)C3c2ccc(Cl)cc2)nc2c(c1NC1(CO)CCC1)[S@](=O)CC2]

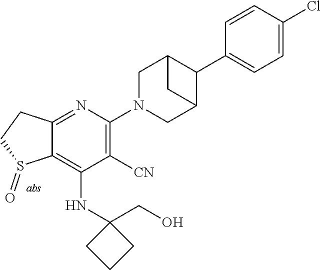 [SMILES: N#Cc1c(N2CC3CC(C2)C3c2ccc(Cl)cc2)nc2c(c1NC1(CO)CCC1)[S@](=O)CC2]

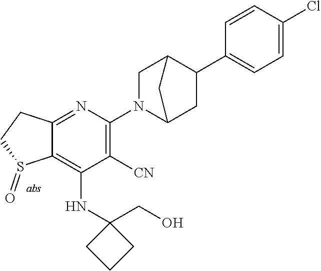 [SMILES: N#Cc1c(N2CC3CC2CC3c2ccc(Cl)cc2)nc2c(c1NC1(CO)CCC1)[S@](=O)CC2]

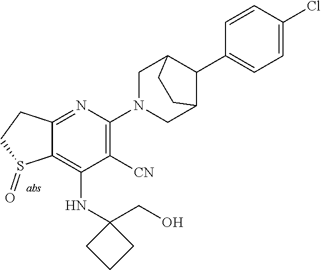 [SMILES: N#Cc1c(N2CC3CCC(C2)C3c2ccc(Cl)cc2)nc2c(c1NC1(CO)CCC1)[S@](=O)CC2]

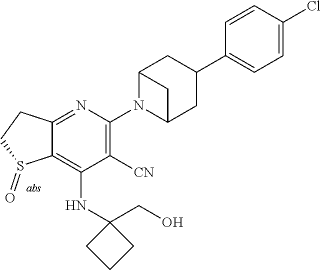 [SMILES: N#Cc1c(N2C3CC(c4ccc(Cl)cc4)CC2C3)nc2c(c1NC1(CO)CCC1)[S@](=O)CC2]

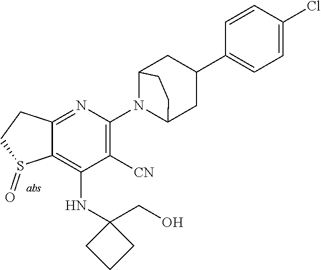 [SMILES: N#Cc1c(N2C3CCC2CC(c2ccc(Cl)cc2)C3)nc2c(c1NC1(CO)CCC1)[S@](=O)CC2]

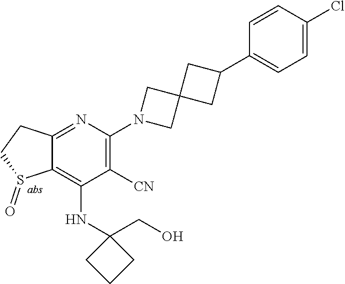 [SMILES: N#Cc1c(N2CC3(CC(c4ccc(Cl)cc4)C3)C2)nc2c(c1NC1(CO)CCC1)[S@](=O)CC2]

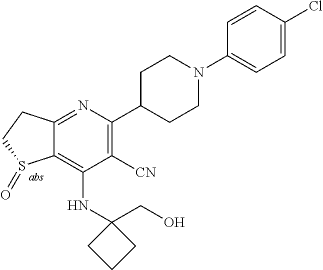 [SMILES: N#Cc1c(C2CCN(c3ccc(Cl)cc3)CC2)nc2c(c1NC1(CO)CCC1)[S@](=O)CC2]

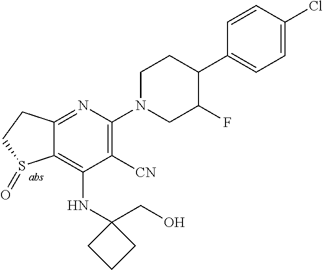 [SMILES: N#Cc1c(N2CCC(c3ccc(Cl)cc3)C(F)C2)nc2c(c1NC1(CO)CCC1)[S@](=O)CC2]

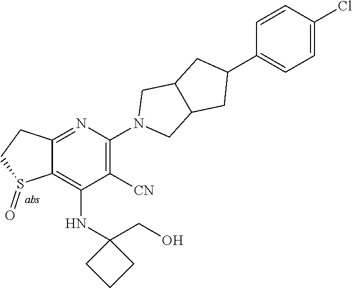 [SMILES: N#Cc1c(N2CC3CC(c4ccc(Cl)cc4)CC3C2)nc2c(c1NC1(CO)CCC1)[S@](=O)CC2]

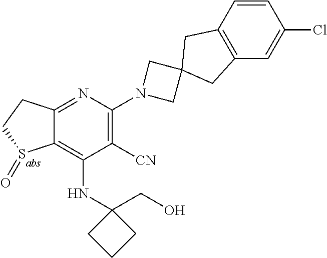 [SMILES: N#Cc1c(N2CC3(Cc4ccc(Cl)cc4C3)C2)nc2c(c1NC1(CO)CCC1)[S@](=O)CC2]

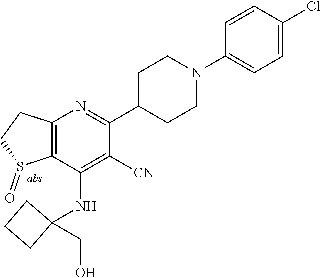 [SMILES: N#Cc1c(C2CCN(c3ccc(Cl)cc3)CC2)nc2c(c1NC1(CO)CCC1)[S@](=O)CC2]

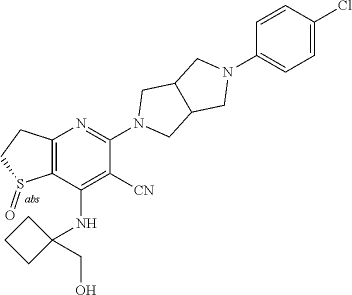 [SMILES: N#Cc1c(N2CC3CN(c4ccc(Cl)cc4)CC3C2)nc2c(c1NC1(CO)CCC1)[S@](=O)CC2]

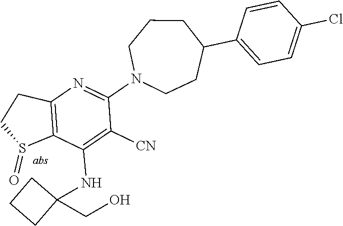 [SMILES: N#Cc1c(N2CCCC(c3ccc(Cl)cc3)CC2)nc2c(c1NC1(CO)CCC1)[S@](=O)CC2]

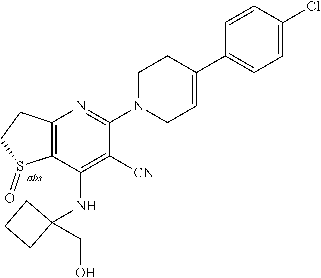 [SMILES: N#Cc1c(N2CC=C(c3ccc(Cl)cc3)CC2)nc2c(c1NC1(CO)CCC1)[S@](=O)CC2]

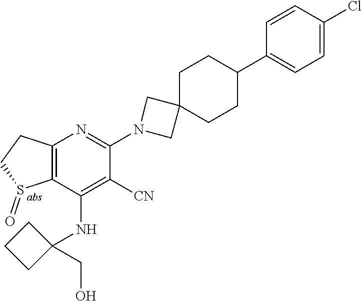 [SMILES: N#Cc1c(N2CC3(CCC(c4ccc(Cl)cc4)CC3)C2)nc2c(c1NC1(CO)CCC1)[S@](=O)CC2]

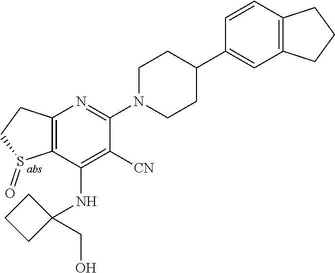 [SMILES: N#Cc1c(N2CCC(c3ccc4c(c3)CCC4)CC2)nc2c(c1NC1(CO)CCC1)[S@](=O)CC2]

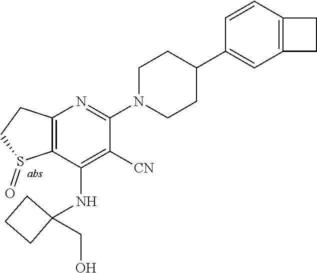 [SMILES: N#Cc1c(N2CCC(c3ccc4c(c3)CC4)CC2)nc2c(c1NC1(CO)CCC1)[S@](=O)CC2]

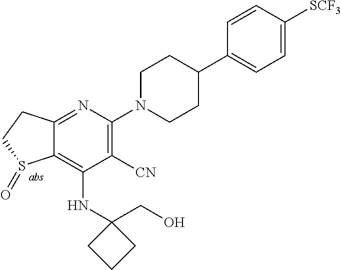

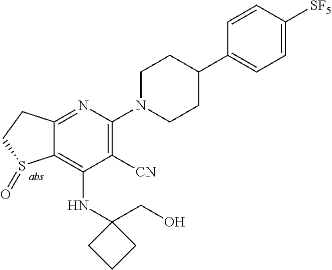

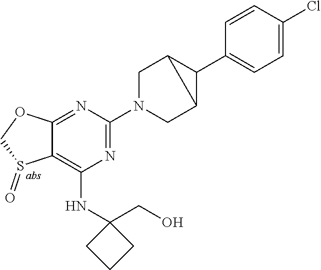 [SMILES: O=[S@@]1COc2nc(N3CC4C(C3)C4c3ccc(Cl)cc3)nc(NC3(CO)CCC3)c21]

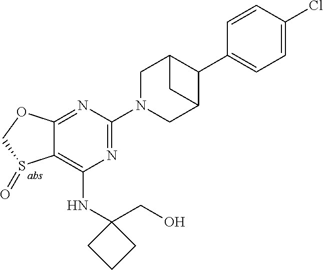 [SMILES: O=[S@@]1COc2nc(N3CC4CC(C3)C4c3ccc(Cl)cc3)nc(NC3(CO)CCC3)c21]

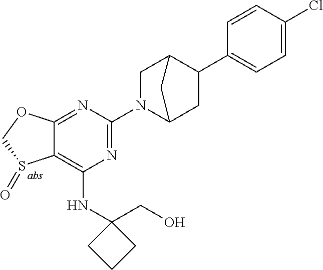 [SMILES: O=[S@@]1COc2nc(N3CC4CC3CC4c3ccc(Cl)cc3)nc(NC3(CO)CCC3)c21]

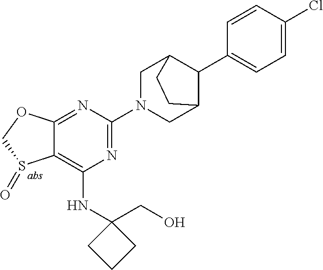 [SMILES: O=[S@@]1COc2nc(N3CC4CCC(C3)C4c3ccc(Cl)cc3)nc(NC3(CO)CCC3)c21]

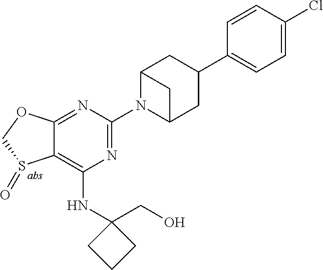 [SMILES: O=[S@@]1COc2nc(N3C4CC(c5ccc(Cl)cc5)CC3C4)nc(NC3(CO)CCC3)c21]

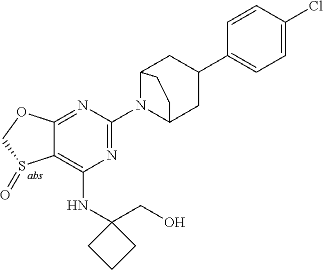 [SMILES: O=[S@@]1COc2nc(N3C4CCC3CC(c3ccc(Cl)cc3)C4)nc(NC3(CO)CCC3)c21]

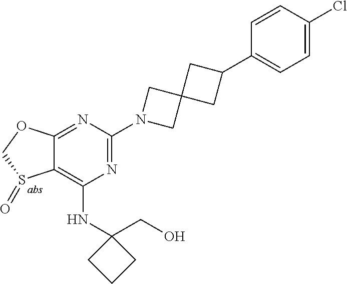 [SMILES: O=[S@@]1COc2nc(N3CC4(CC(c5ccc(Cl)cc5)C4)C3)nc(NC3(CO)CCC3)c21]

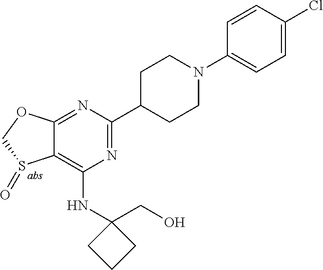 [SMILES: O=[S@@]1COc2nc(C3CCN(c4ccc(Cl)cc4)CC3)nc(NC3(CO)CCC3)c21]

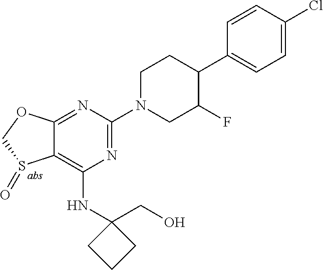 [SMILES: O=[S@@]1COc2nc(N3CCC(c4ccc(Cl)cc4)C(F)C3)nc(NC3(CO)CCC3)c21]

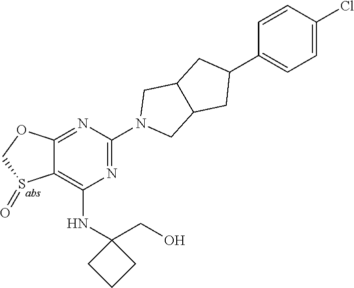 [SMILES: O=[S@@]1COc2nc(N3CC4CC(c5ccc(Cl)cc5)CC4C3)nc(NC3(CO)CCC3)c21]

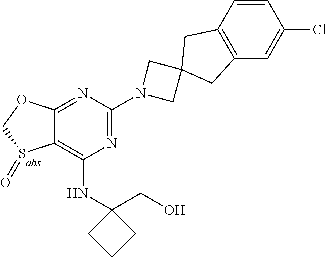 [SMILES: O=[S@@]1COc2nc(N3CC4(Cc5ccc(Cl)cc5C4)C3)nc(NC3(CO)CCC3)c21]

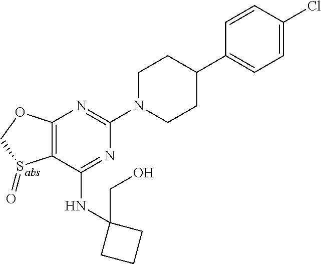 [SMILES: O=[S@@]1COc2nc(N3CCC(c4ccc(Cl)cc4)CC3)nc(NC3(CO)CCC3)c21]

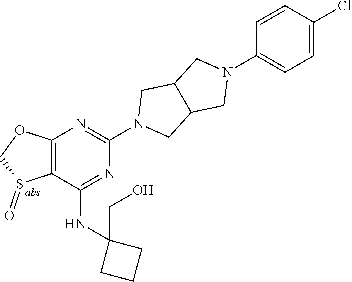 [SMILES: O=[S@@]1COc2nc(N3CC4CN(c5ccc(Cl)cc5)CC4C3)nc(NC3(CO)CCC3)c21]

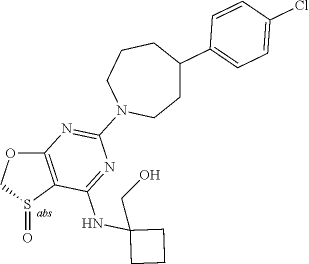 [SMILES: O=[S@@]1COc2nc(N3CCCC(c4ccc(Cl)cc4)CC3)nc(NC3(CO)CCC3)c21]

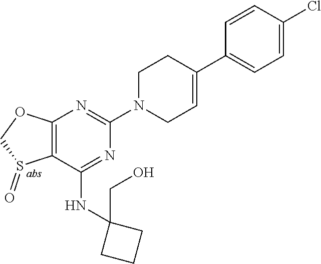 [SMILES: O=[S@@]1COc2nc(N3CC=C(c4ccc(Cl)cc4)CC3)nc(NC3(CO)CCC3)c21]

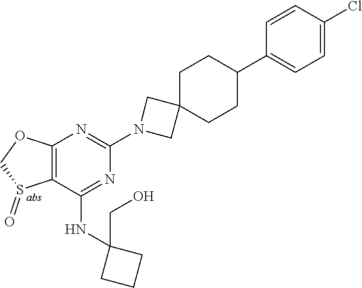 [SMILES: O=[S@@]1COc2nc(N3CC4(CCC(c5ccc(Cl)cc5)CC4)C3)nc(NC3(CO)CCC3)c21]

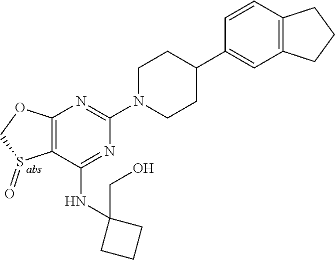 [SMILES: O=[S@@]1COc2nc(N3CCC(c4ccc5c(c4)CCC5)CC3)nc(NC3(CO)CCC3)c21]

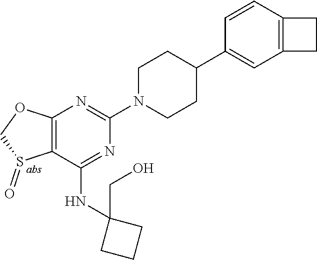 [SMILES: O=[S@@]1COc2nc(N3CCC(c4ccc5c(c4)CC5)CC3)nc(NC3(CO)CCC3)c21]

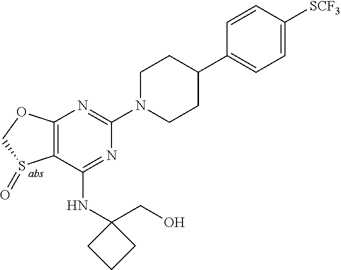

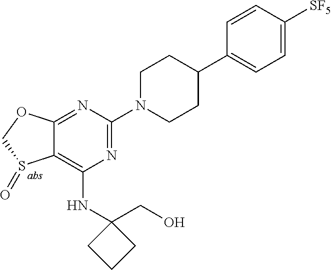

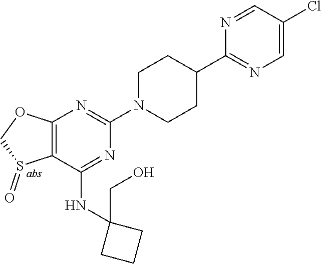 [SMILES: O=[S@@]1COc2nc(N3CCC(c4ncc(Cl)cn4)CC3)nc(NC3(CO)CCC3)c21]

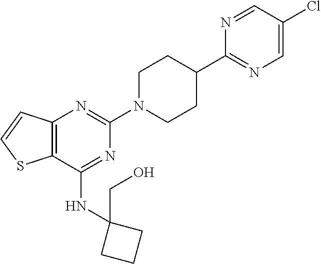 [SMILES: OCC1(Nc2nc(N3CCC(c4ncc(Cl)cn4)CC3)nc3ccsc23)CCC1]

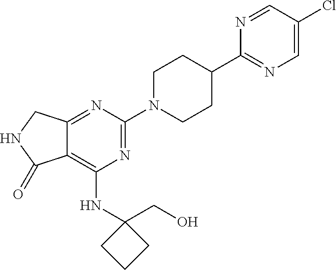 [SMILES: O=C1NCc2nc(N3CCC(c4ncc(Cl)cn4)CC3)nc(NC3(CO)CCC3)c21]

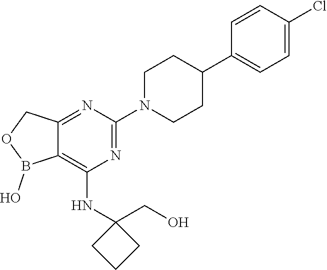 [SMILES: OCC1(Nc2nc(N3CCC(c4ccc(Cl)cc4)CC3)nc3c2B(O)OC3)CCC1]

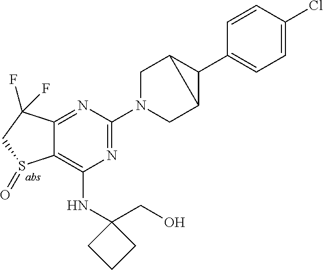 [SMILES: O=[S@@]1CC(F)(F)c2nc(N3CC4C(C3)C4c3ccc(Cl)cc3)nc(NC3(CO)CCC3)c21]

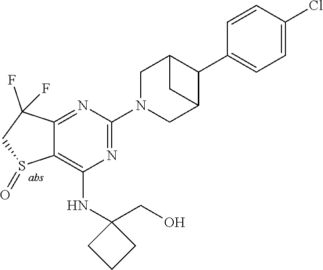 [SMILES: O=[S@@]1CC(F)(F)c2nc(N3CC4CC(C3)C4c3ccc(Cl)cc3)nc(NC3(CO)CCC3)c21]

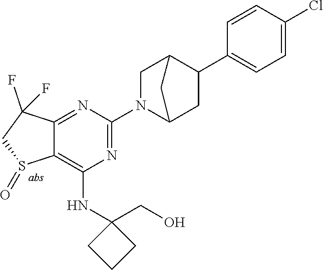 [SMILES: O=[S@@]1CC(F)(F)c2nc(N3CC4CC3CC4c3ccc(Cl)cc3)nc(NC3(CO)CCC3)c21]

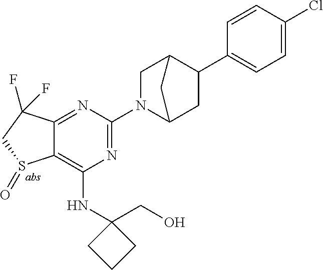 [SMILES: O=[S@@]1CC(F)(F)c2nc(N3CC4CC3CC4c3ccc(Cl)cc3)nc(NC3(CO)CCC3)c21]

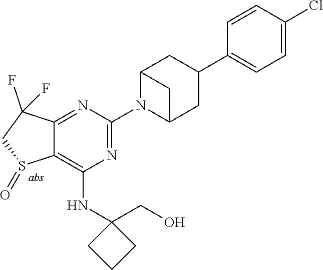 [SMILES: O=[S@@]1CC(F)(F)c2nc(N3C4CC(c5ccc(Cl)cc5)CC3C4)nc(NC3(CO)CCC3)c21]

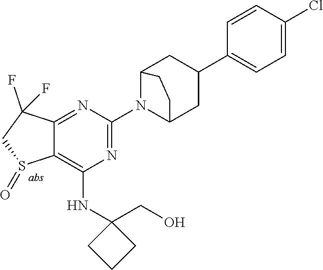 [SMILES: O=[S@@]1CC(F)(F)c2nc(N3C4CCC3CC(c3ccc(Cl)cc3)C4)nc(NC3(CO)CCC3)c21]

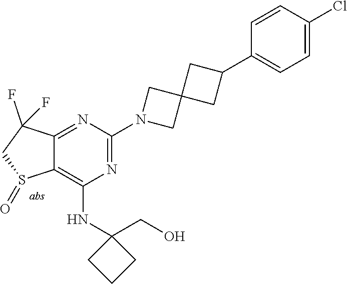 [SMILES: O=[S@@]1CC(F)(F)c2nc(N3CC4(CC(c5ccc(Cl)cc5)C4)C3)nc(NC3(CO)CCC3)c21]

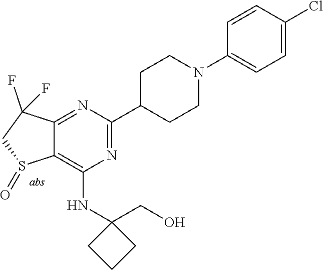 [SMILES: O=[S@@]1CC(F)(F)c2nc(C3CCN(c4ccc(Cl)cc4)CC3)nc(NC3(CO)CCC3)c21]

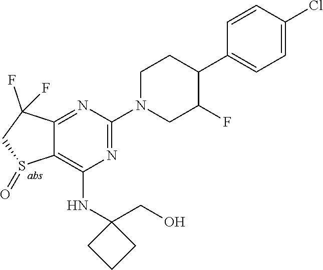 [SMILES: O=[S@@]1CC(F)(F)c2nc(N3CCC(c4ccc(Cl)cc4)C(F)C3)nc(NC3(CO)CCC3)c21]

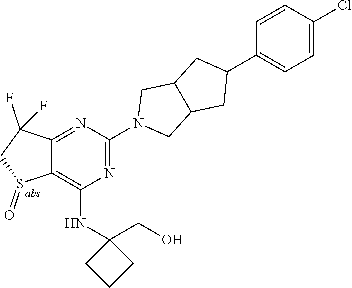 [SMILES: O=[S@@]1CC(F)(F)c2nc(N3CC4CC(c5ccc(Cl)cc5)CC4C3)nc(NC3(CO)CCC3)c21]

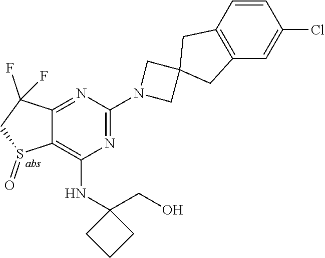 [SMILES: O=[S@@]1CC(F)(F)c2nc(N3CC4(Cc5ccc(Cl)cc5C4)C3)nc(NC3(CO)CCC3)c21]

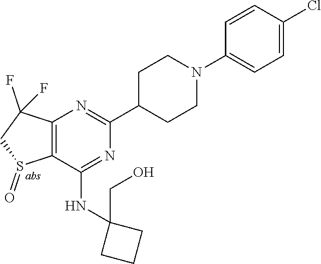 [SMILES: O=[S@@]1CC(F)(F)c2nc(C3CCN(c4ccc(Cl)cc4)CC3)nc(NC3(CO)CCC3)c21]

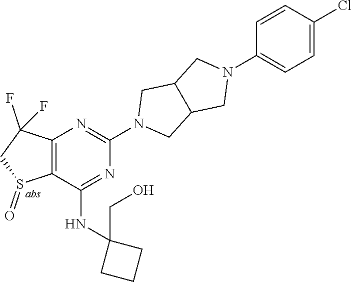 [SMILES: O=[S@@]1CC(F)(F)c2nc(N3CC4CN(c5ccc(Cl)cc5)CC4C3)nc(NC3(CO)CCC3)c21]

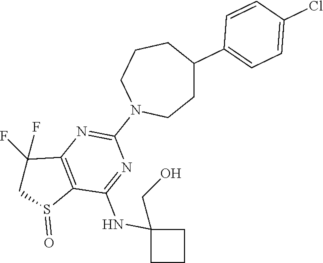 [SMILES: O=[S@@]1CC(F)(F)c2nc(N3CCCC(c4ccc(Cl)cc4)CC3)nc(NC3(CO)CCC3)c21]

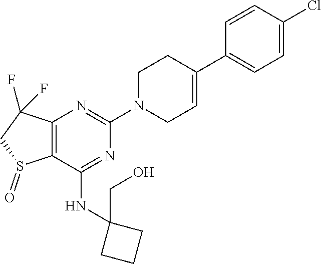 [SMILES: O=[S@@]1CC(F)(F)c2nc(N3CC=C(c4ccc(Cl)cc4)CC3)nc(NC3(CO)CCC3)c21]

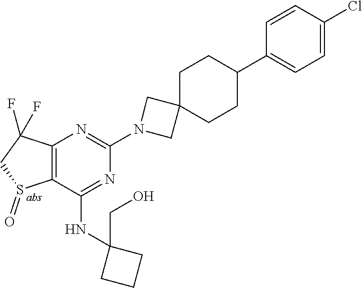 [SMILES: O=[S@@]1CC(F)(F)c2nc(N3CC4(CCC(c5ccc(Cl)cc5)CC4)C3)nc(NC3(CO)CCC3)c21]

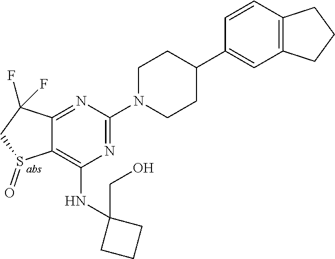 [SMILES: O=[S@@]1CC(F)(F)c2nc(N3CCC(c4ccc5c(c4)CCC5)CC3)nc(NC3(CO)CCC3)c21]

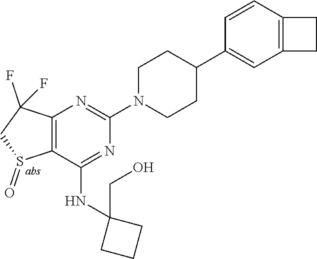 [SMILES: O=[S@@]1CC(F)(F)c2nc(N3CCC(c4ccc5c(c4)CC5)CC3)nc(NC3(CO)CCC3)c21]

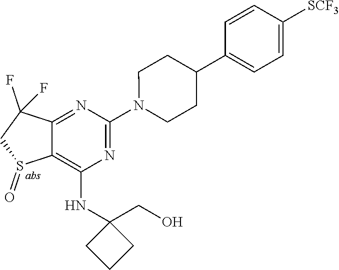

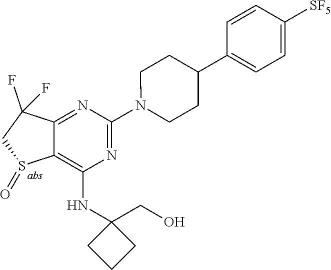

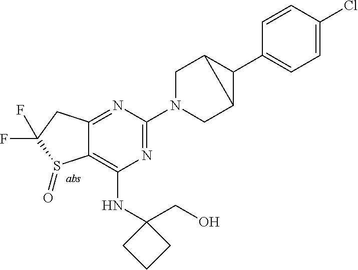 [SMILES: O=[S@]1c2c(nc(N3CC4C(C3)C4c3ccc(Cl)cc3)nc2NC2(CO)CCC2)CC1(F)F]

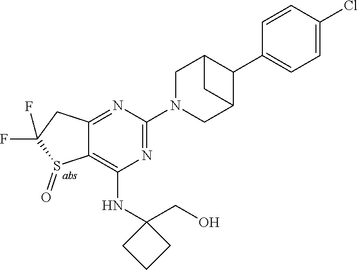 [SMILES: O=[S@]1c2c(nc(N3CC4CC(C3)C4c3ccc(Cl)cc3)nc2NC2(CO)CCC2)CC1(F)F]

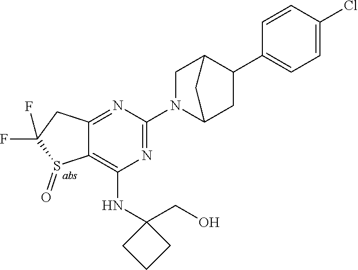 [SMILES: O=[S@]1c2c(nc(N3CC4CC3CC4c3ccc(Cl)cc3)nc2NC2(CO)CCC2)CC1(F)F]

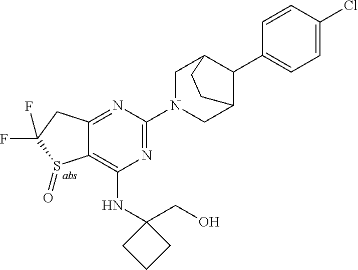 [SMILES: O=[S@]1c2c(nc(N3CC4CCC(C3)C4c3ccc(Cl)cc3)nc2NC2(CO)CCC2)CC1(F)F]

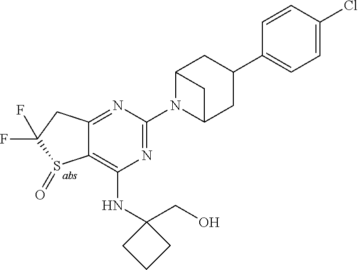 [SMILES: O=[S@]1c2c(nc(N3C4CC(c5ccc(Cl)cc5)CC3C4)nc2NC2(CO)CCC2)CC1(F)F]

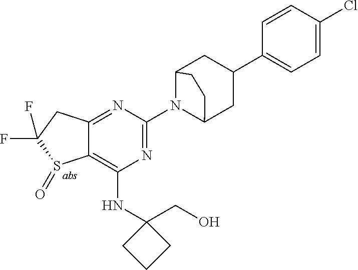 [SMILES: O=[S@]1c2c(nc(N3C4CCC3CC(c3ccc(Cl)cc3)C4)nc2NC2(CO)CCC2)CC1(F)F]

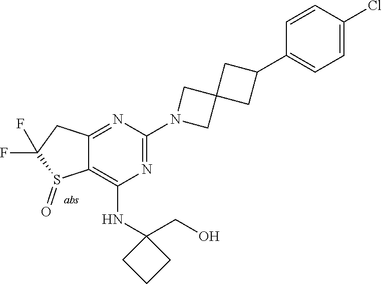 [SMILES: O=[S@]1c2c(nc(N3CC4(CC(c5ccc(Cl)cc5)C4)C3)nc2NC2(CO)CCC2)CC1(F)F]

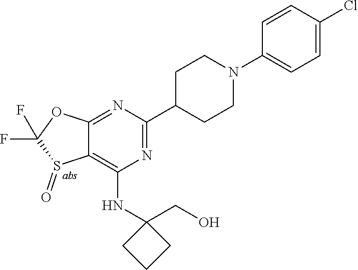 [SMILES: O=[S@]1c2c(NC3(CO)CCC3)nc(C3CCN(c4ccc(Cl)cc4)CC3)nc2OC1(F)F]

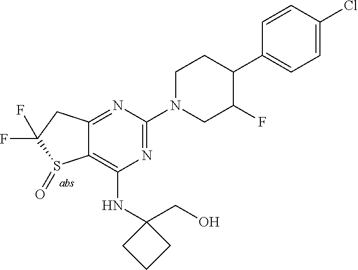 [SMILES: O=[S@]1c2c(nc(N3CCC(c4ccc(Cl)cc4)C(F)C3)nc2NC2(CO)CCC2)CC1(F)F]

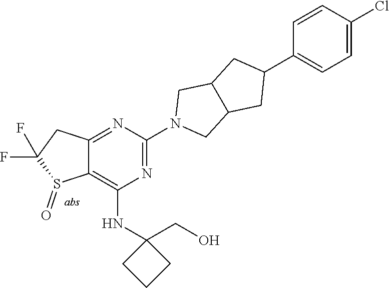 [SMILES: O=[S@]1c2c(nc(N3CC4CC(c5ccc(Cl)cc5)CC4C3)nc2NC2(CO)CCC2)CC1(F)F]

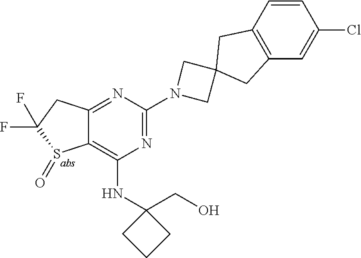 [SMILES: O=[S@]1c2c(nc(N3CC4(Cc5ccc(Cl)cc5C4)C3)nc2NC2(CO)CCC2)CC1(F)F]

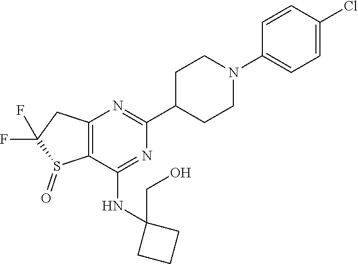 [SMILES: O=[S@]1c2c(nc(C3CCN(c4ccc(Cl)cc4)CC3)nc2NC2(CO)CCC2)CC1(F)F]

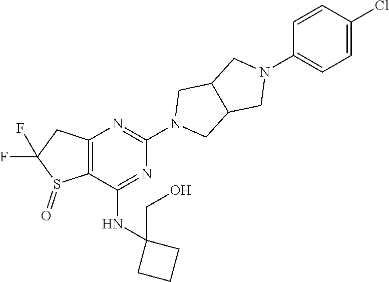 [SMILES: O=S1c2c(nc(N3CC4CN(c5ccc(Cl)cc5)CC4C3)nc2NC2(CO)CCC2)CC1(F)F]

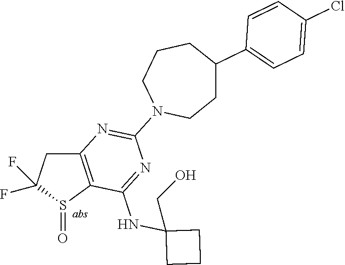 [SMILES: O=[S@]1c2c(nc(N3CCCC(c4ccc(Cl)cc4)CC3)nc2NC2(CO)CCC2)CC1(F)F]

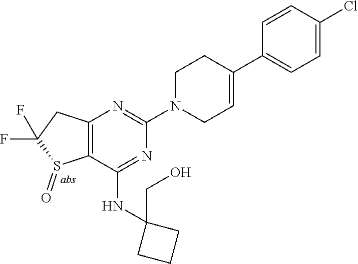 [SMILES: O=[S@]1c2c(nc(N3CC=C(c4ccc(Cl)cc4)CC3)nc2NC2(CO)CCC2)CC1(F)F]

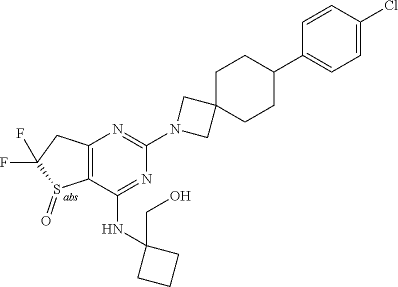 [SMILES: O=[S@]1c2c(nc(N3CC4(CCC(c5ccc(Cl)cc5)CC4)C3)nc2NC2(CO)CCC2)CC1(F)F]

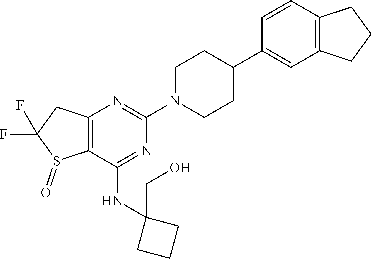 [SMILES: O=S1c2c(nc(N3CCC(c4ccc5c(c4)CCC5)CC3)nc2NC2(CO)CCC2)CC1(F)F]

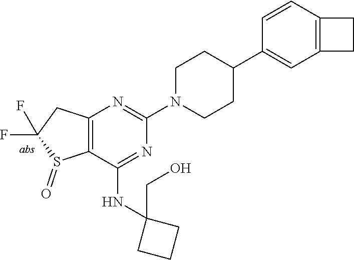

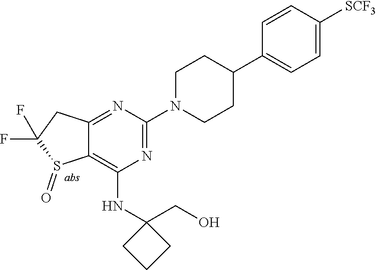

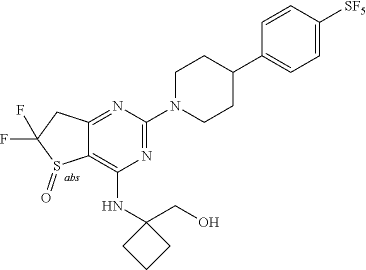

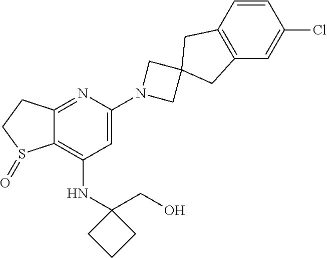 [SMILES: O=S1CCc2nc(N3CC4(Cc5ccc(Cl)cc5C4)C3)cc(NC3(CO)CCC3)c21]

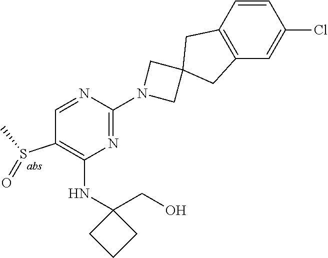 [SMILES: C[S@@](=O)c1cnc(N2CC3(Cc4ccc(Cl)cc4C3)C2)nc1NC1(CO)CCC1]

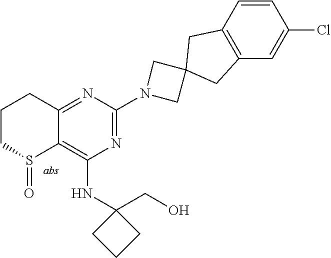 [SMILES: O=[S@@]1CCCc2nc(N3CC4(Cc5ccc(Cl)cc5C4)C3)nc(NC3(CO)CCC3)c21]

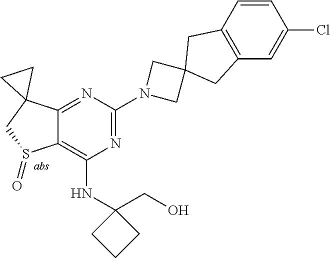 [SMILES: O=[S@@]1CC2(CC2)c2nc(N3CC4(Cc5ccc(Cl)cc5C4)C3)nc(NC3(CO)CCC3)c21]

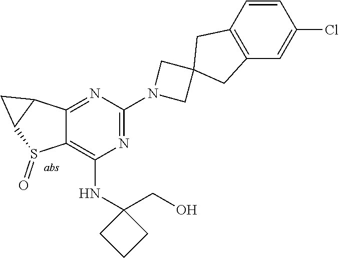

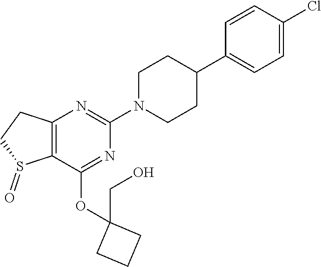 [SMILES: O=[S@@]1CCc2nc(N3CCC(c4ccc(Cl)cc4)CC3)nc(OC3(CO)CCC3)c21]

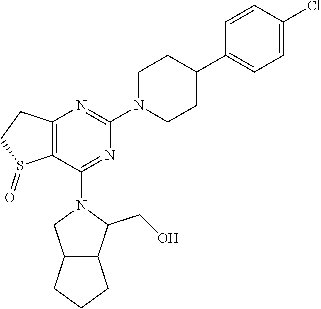 [SMILES: O=[S@@]1CCc2nc(N3CCC(c4ccc(Cl)cc4)CC3)nc(N3CC4CCCC4C3CO)c21]

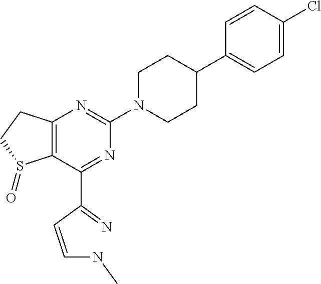 [SMILES: CN/C=C\C(=N)c1nc(N2CCC(c3ccc(Cl)cc3)CC2)nc2c1[S@](=O)CC2]

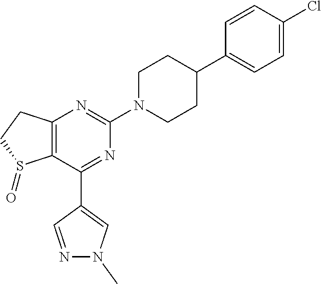 [SMILES: Cn1cc(-c2nc(N3CCC(c4ccc(Cl)cc4)CC3)nc3c2[S@](=O)CC3)cn1]

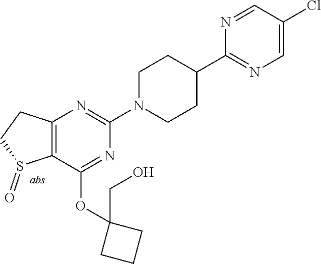 [SMILES: O=[S@@]1CCc2nc(N3CCC(c4ncc(Cl)cn4)CC3)nc(OC3(CO)CCC3)c21]

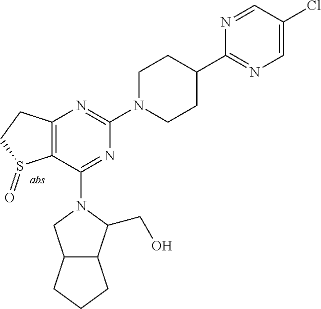 [SMILES: O=[S@@]1CCc2nc(N3CCC(c4ncc(Cl)cn4)CC3)nc(N3CC4CCCC4C3CO)c21]

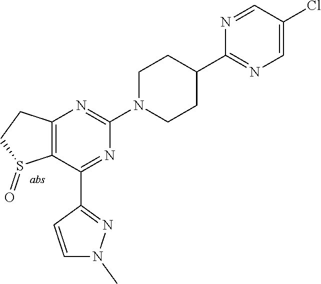 [SMILES: Cn1ccc(-c2nc(N3CCC(c4ncc(Cl)cn4)CC3)nc3c2[S@](=O)CC3)n1]

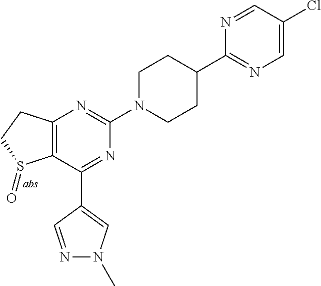 [SMILES: Cn1cc(-c2nc(N3CCC(c4ncc(Cl)cn4)CC3)nc3c2[S@](=O)CC3)cn1]

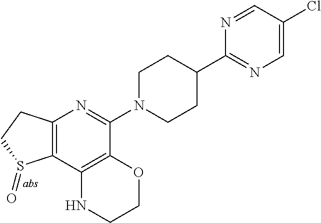 [SMILES: O=[S@@]1CCc2nc(N3CCC(c4ncc(Cl)cn4)CC3)c3c(c21)NCCO3]

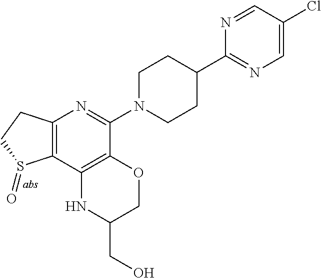 [SMILES: O=[S@@]1CCc2nc(N3CCC(c4ncc(Cl)cn4)CC3)c3c(c21)NC(CO)CO3]

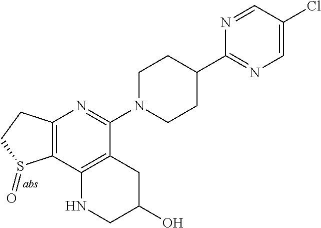 [SMILES: O=[S@@]1CCc2nc(N3CCC(c4ncc(Cl)cn4)CC3)c3c(c21)NCC(O)C3]

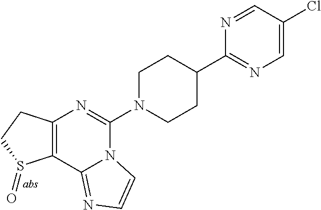 [SMILES: O=[S@@]1CCc2nc(N3CCC(c4ncc(Cl)cn4)CC3)n3ccnc3c21]

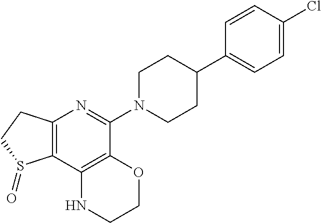 [SMILES: O=[S@@]1CCc2nc(N3CCC(c4ccc(Cl)cc4)CC3)c3c(c21)NCCO3]

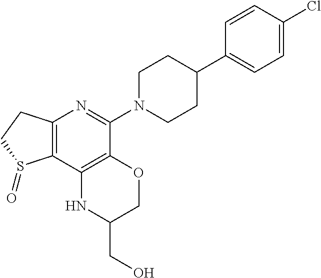 [SMILES: O=[S@@]1CCc2nc(N3CCC(c4ccc(Cl)cc4)CC3)c3c(c21)NC(CO)CO3]

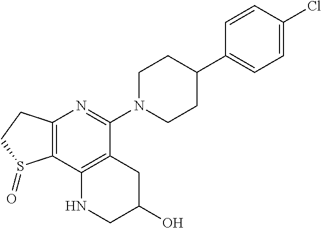 [SMILES: O=[S@@]1CCc2nc(N3CCC(c4ccc(Cl)cc4)CC3)c3c(c21)NCC(O)C3]

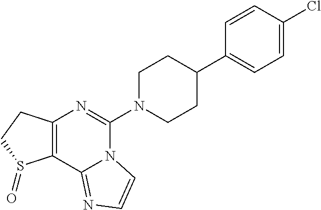 [SMILES: O=[S@@]1CCc2nc(N3CCC(c4ccc(Cl)cc4)CC3)n3ccnc3c21]

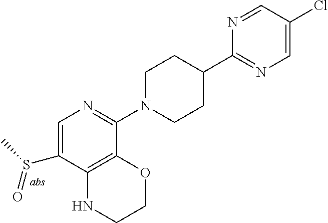 [SMILES: C[S@@](=O)c1cnc(N2CCC(c3ncc(Cl)cn3)CC2)c2c1NCCO2]

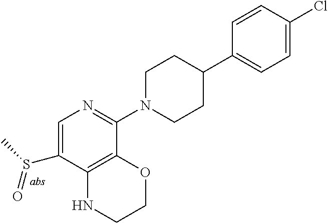 [SMILES: C[S@@](=O)c1cnc(N2CCC(c3ccc(Cl)cc3)CC2)c2c1NCCO2]

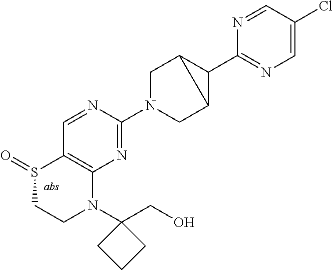 [SMILES: O=[S@]1CCN(C2(CO)CCC2)c2nc(N3CC4C(C3)C4c3ncc(Cl)cn3)ncc21]

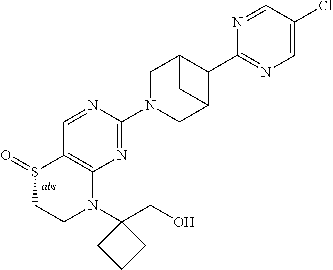 [SMILES: O=[S@]1CCN(C2(CO)CCC2)c2nc(N3CC4CC(C3)C4c3ncc(Cl)cn3)ncc21]

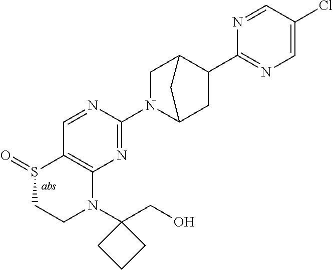 [SMILES: O=[S@]1CCN(C2(CO)CCC2)c2nc(N3CC4CC3CC4c3ncc(Cl)cn3)ncc21]

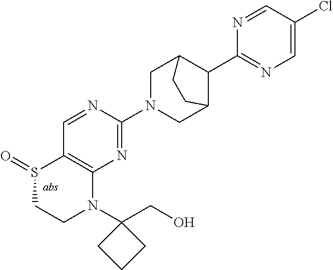 [SMILES: O=[S@]1CCN(C2(CO)CCC2)c2nc(N3CC4CCC(C3)C4c3ncc(Cl)cn3)ncc21]

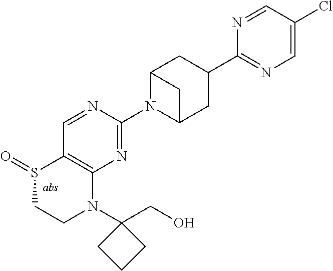 [SMILES: O=[S@]1CCN(C2(CO)CCC2)c2nc(N3C4CC(c5ncc(Cl)cn5)CC3C4)ncc21]

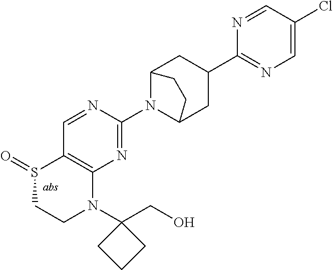 [SMILES: O=[S@]1CCN(C2(CO)CCC2)c2nc(N3C4CCC3CC(c3ncc(Cl)cn3)C4)ncc21]

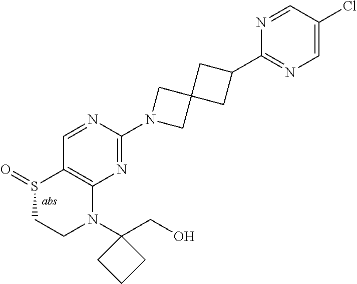 [SMILES: O=[S@]1CCN(C2(CO)CCC2)c2nc(N3CC4(CC(c5ncc(Cl)cn5)C4)C3)ncc21]

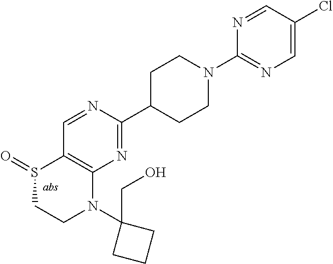 [SMILES: O=[S@]1CCN(C2(CO)CCC2)c2nc(C3CCN(c4ncc(Cl)cn4)CC3)ncc21]

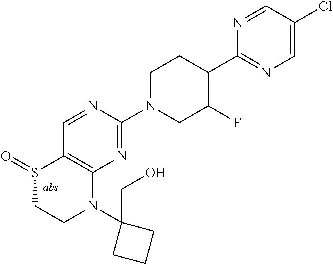 [SMILES: O=[S@]1CCN(C2(CO)CCC2)c2nc(N3CCC(c4ncc(Cl)cn4)C(F)C3)ncc21]

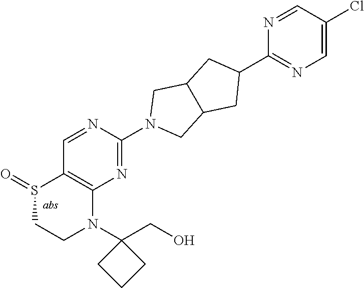 [SMILES: O=[S@]1CCN(C2(CO)CCC2)c2nc(N3CC4CC(c5ncc(Cl)cn5)CC4C3)ncc21]

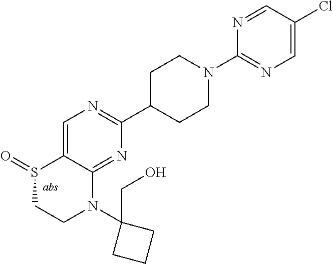 [SMILES: O=[S@]1CCN(C2(CO)CCC2)c2nc(C3CCN(c4ncc(Cl)cn4)CC3)ncc21]

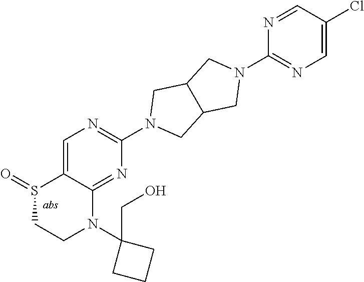 [SMILES: O=[S@]1CCN(C2(CO)CCC2)c2nc(N3CC4CN(c5ncc(Cl)cn5)CC4C3)ncc21]

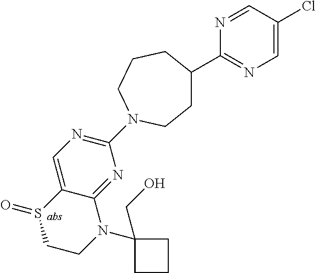 [SMILES: O=[S@]1CCN(C2(CO)CCC2)c2nc(N3CCCC(c4ncc(Cl)cn4)CC3)ncc21]

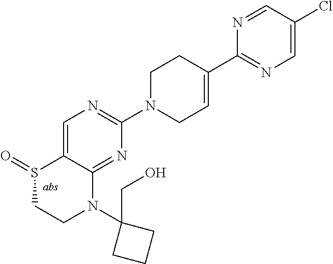 [SMILES: O=[S@]1CCN(C2(CO)CCC2)c2nc(N3CC=C(c4ncc(Cl)cn4)CC3)ncc21]

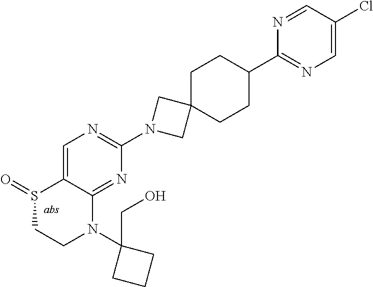 [SMILES: O=[S@]1CCN(C2(CO)CCC2)c2nc(N3CC4(CCC(c5ncc(Cl)cn5)CC4)C3)ncc21]

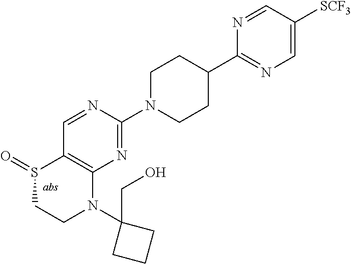

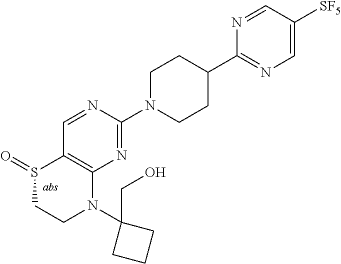

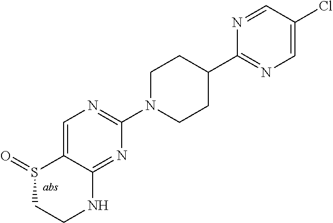 [SMILES: O=[S@]1CCNc2nc(N3CCC(c4ncc(Cl)cn4)CC3)ncc21]

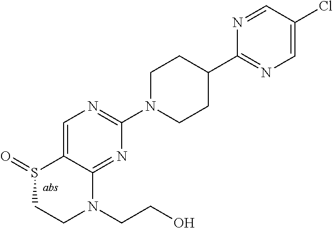 [SMILES: O=[S@]1CCN(CCO)c2nc(N3CCC(c4ncc(Cl)cn4)CC3)ncc21]

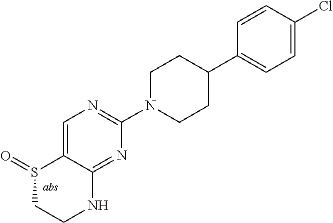 [SMILES: O=[S@]1CCNc2nc(N3CCC(c4ccc(Cl)cc4)CC3)ncc21]

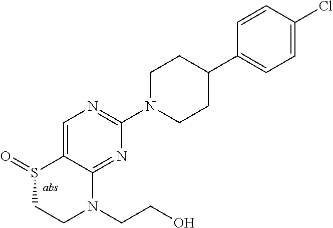 [SMILES: O=[S@]1CCN(CCO)c2nc(N3CCC(c4ccc(Cl)cc4)CC3)ncc21]

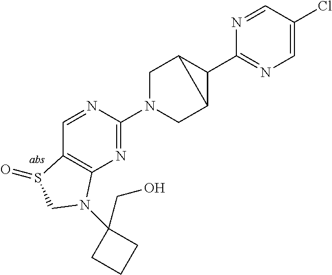 [SMILES: O=[S@]1CN(C2(CO)CCC2)c2nc(N3CC4C(C3)C4c3ncc(Cl)cn3)ncc21]

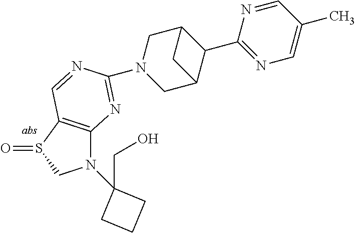 [SMILES: Cc1cnc(C2C3CC2CN(c2ncc4c(n2)N(C2(CO)CCC2)C[S@@]4=O)C3)nc1]

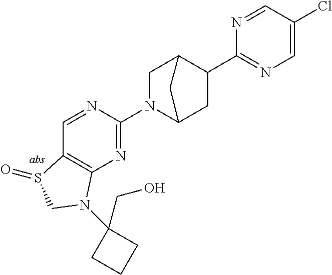 [SMILES: O=[S@]1CN(C2(CO)CCC2)c2nc(N3CC4CC3CC4c3ncc(Cl)cn3)ncc21]

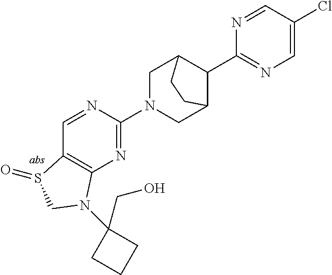 [SMILES: O=[S@]1CN(C2(CO)CCC2)c2nc(N3CC4CCC(C3)C4c3ncc(Cl)cn3)ncc21]

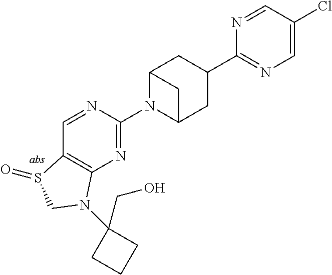 [SMILES: O=[S@]1CN(C2(CO)CCC2)c2nc(N3C4CC(c5ncc(Cl)cn5)CC3C4)ncc21]

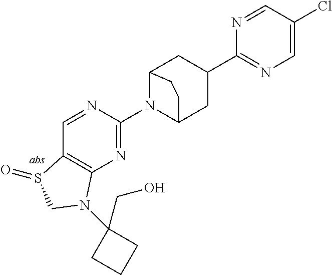 [SMILES: O=[S@]1CN(C2(CO)CCC2)c2nc(N3C4CCC3CC(c3ncc(Cl)cn3)C4)ncc21]

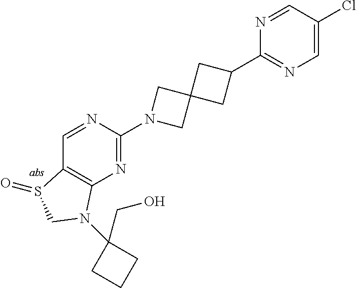 [SMILES: O=[S@]1CN(C2(CO)CCC2)c2nc(N3CC4(CC(c5ncc(Cl)cn5)C4)C3)ncc21]

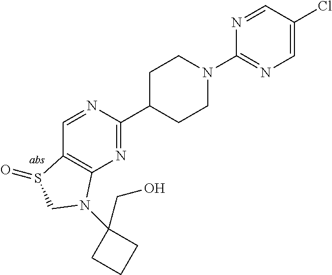 [SMILES: O=[S@]1CN(C2(CO)CCC2)c2nc(C3CCN(c4ncc(Cl)cn4)CC3)ncc21]

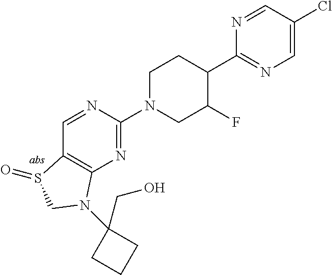 [SMILES: O=[S@]1CN(C2(CO)CCC2)c2nc(N3CCC(c4ncc(Cl)cn4)C(F)C3)ncc21]

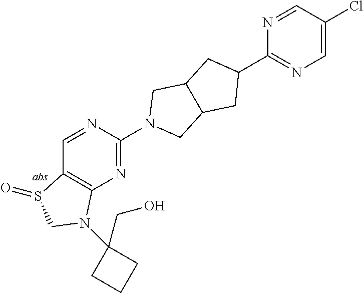 [SMILES: O=[S@]1CN(C2(CO)CCC2)c2nc(N3CC4CC(c5ncc(Cl)cn5)CC4C3)ncc21]

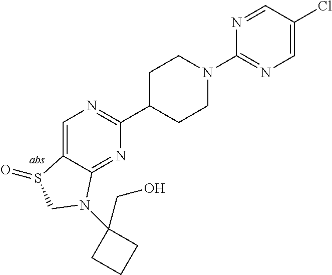 [SMILES: O=[S@]1CN(C2(CO)CCC2)c2nc(C3CCN(c4ncc(Cl)cn4)CC3)ncc21]

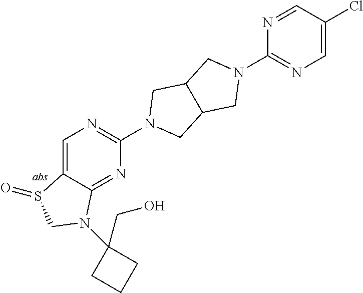 [SMILES: O=[S@]1CN(C2(CO)CCC2)c2nc(N3CC4CN(c5ncc(Cl)cn5)CC4C3)ncc21]

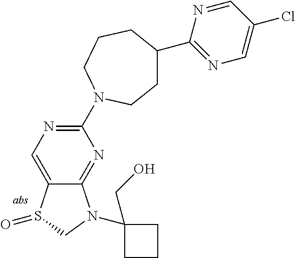 [SMILES: O=[S@]1CN(C2(CO)CCC2)c2nc(N3CCCC(c4ncc(Cl)cn4)CC3)ncc21]

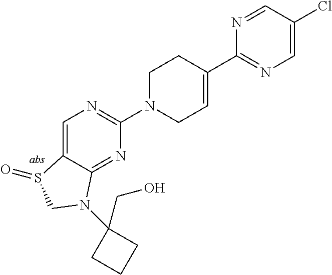 [SMILES: O=[S@]1CN(C2(CO)CCC2)c2nc(N3CC=C(c4ncc(Cl)cn4)CC3)ncc21]

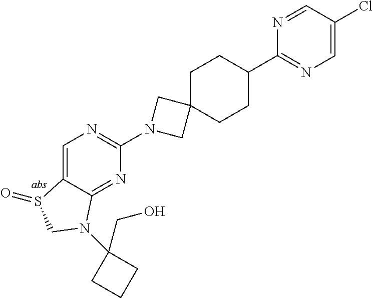 [SMILES: O=[S@]1CN(C2(CO)CCC2)c2nc(N3CC4(CCC(c5ncc(Cl)cn5)CC4)C3)ncc21]

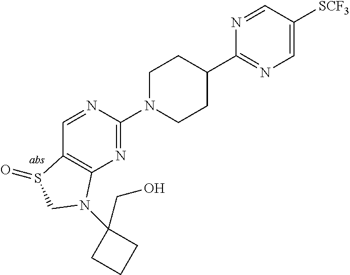

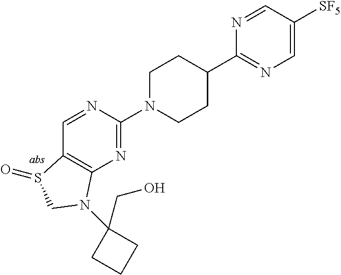

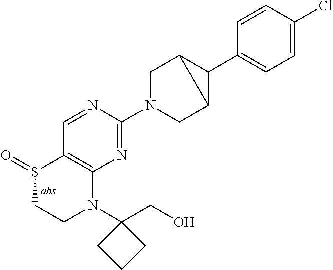 [SMILES: O=[S@]1CCN(C2(CO)CCC2)c2nc(N3CC4C(C3)C4c3ccc(Cl)cc3)ncc21]

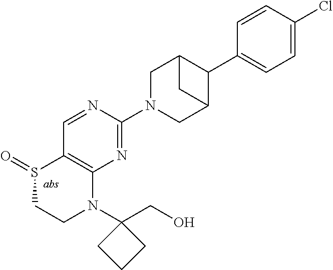 [SMILES: O=[S@]1CCN(C2(CO)CCC2)c2nc(N3CC4CC(C3)C4c3ccc(Cl)cc3)ncc21]

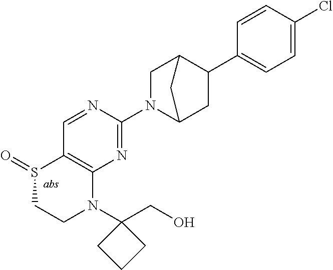 [SMILES: O=[S@]1CCN(C2(CO)CCC2)c2nc(N3CC4CC3CC4c3ccc(Cl)cc3)ncc21]

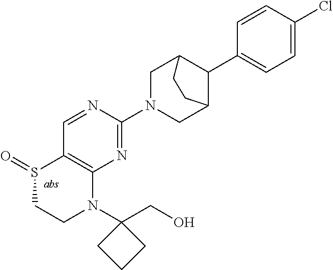 [SMILES: O=[S@]1CCN(C2(CO)CCC2)c2nc(N3CC4CCC(C3)C4c3ccc(Cl)cc3)ncc21]

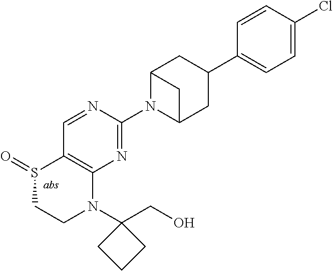 [SMILES: O=[S@]1CCN(C2(CO)CCC2)c2nc(N3C4CC(c5ccc(Cl)cc5)CC3C4)ncc21]

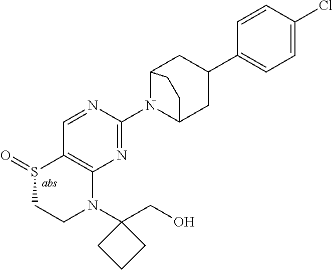 [SMILES: O=[S@]1CCN(C2(CO)CCC2)c2nc(N3C4CCC3CC(c3ccc(Cl)cc3)C4)ncc21]

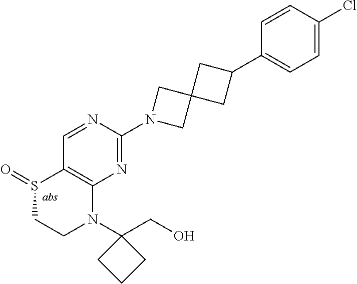 [SMILES: O=[S@]1CCN(C2(CO)CCC2)c2nc(N3CC4(CC(c5ccc(Cl)cc5)C4)C3)ncc21]

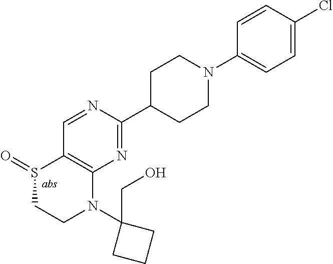 [SMILES: O=[S@]1CCN(C2(CO)CCC2)c2nc(C3CCN(c4ccc(Cl)cc4)CC3)ncc21]

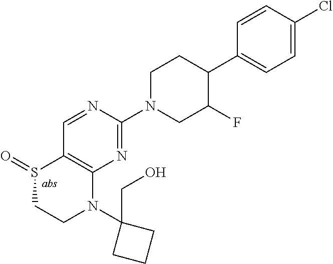 [SMILES: O=[S@]1CCN(C2(CO)CCC2)c2nc(N3CCC(c4ccc(Cl)cc4)C(F)C3)ncc21]

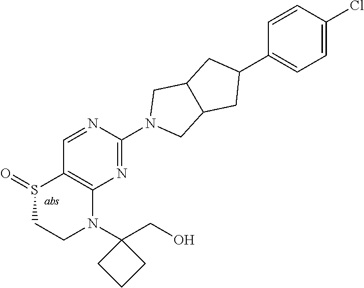 [SMILES: O=[S@]1CCN(C2(CO)CCC2)c2nc(N3CC4CC(c5ccc(Cl)cc5)CC4C3)ncc21]

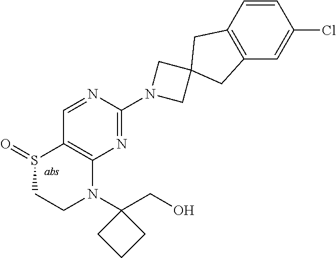 [SMILES: O=[S@]1CCN(C2(CO)CCC2)c2nc(N3CC4(Cc5ccc(Cl)cc5C4)C3)ncc21]

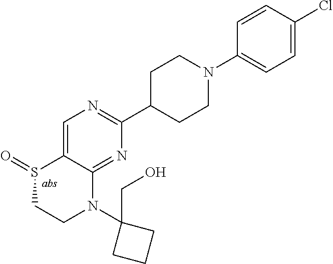 [SMILES: O=[S@]1CCN(C2(CO)CCC2)c2nc(C3CCN(c4ccc(Cl)cc4)CC3)ncc21]

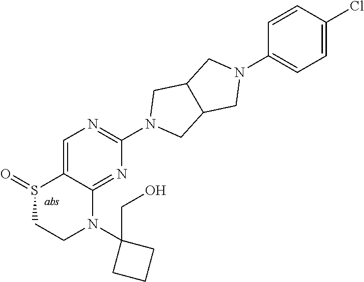 [SMILES: O=[S@]1CCN(C2(CO)CCC2)c2nc(N3CC4CN(c5ccc(Cl)cc5)CC4C3)ncc21]

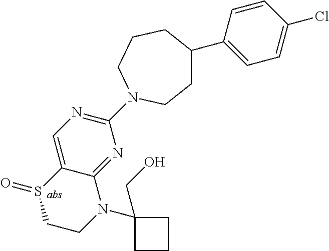 [SMILES: O=[S@]1CCN(C2(CO)CCC2)c2nc(N3CCCC(c4ccc(Cl)cc4)CC3)ncc21]

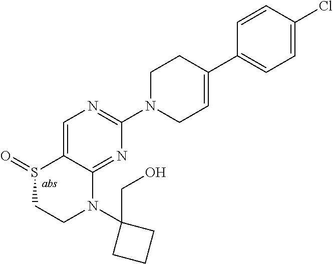 [SMILES: O=[S@]1CCN(C2(CO)CCC2)c2nc(N3CC=C(c4ccc(Cl)cc4)CC3)ncc21]

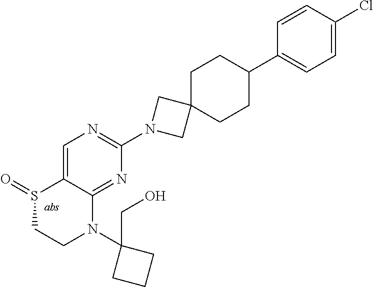 [SMILES: O=[S@]1CCN(C2(CO)CCC2)c2nc(N3CC4(CCC(c5ccc(Cl)cc5)CC4)C3)ncc21]

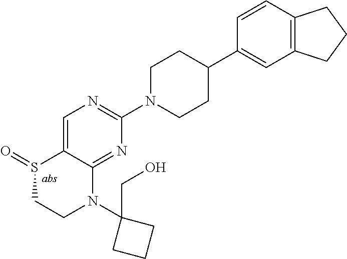 [SMILES: O=[S@]1CCN(C2(CO)CCC2)c2nc(N3CCC(c4ccc5c(c4)CCC5)CC3)ncc21]

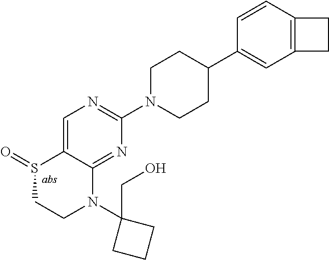 [SMILES: O=[S@]1CCN(C2(CO)CCC2)c2nc(N3CCC(c4ccc5c(c4)CC5)CC3)ncc21]

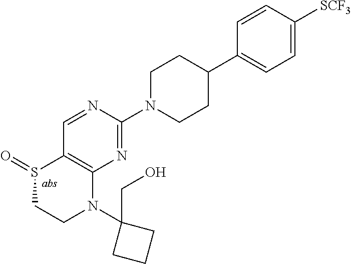

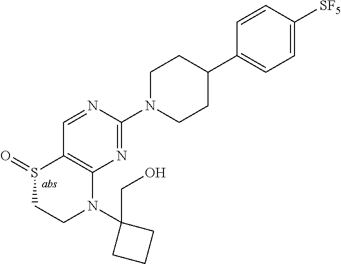

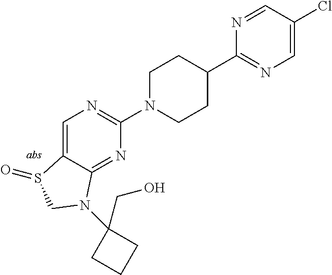 [SMILES: O=[S@]1CN(C2(CO)CCC2)c2nc(N3CCC(c4ncc(Cl)cn4)CC3)ncc21]

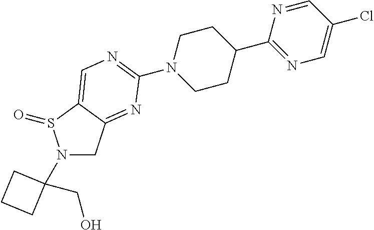 [SMILES: O=S1c2cnc(N3CCC(c4ncc(Cl)cn4)CC3)nc2CN1C1(CO)CCC1]

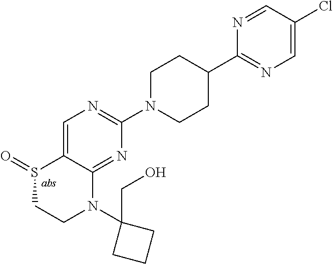 [SMILES: O=[S@]1CCN(C2(CO)CCC2)c2nc(N3CCC(c4ncc(Cl)cn4)CC3)ncc21]

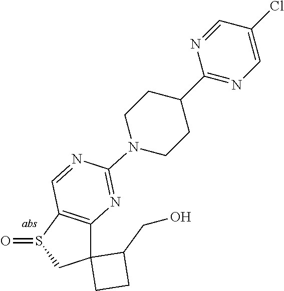

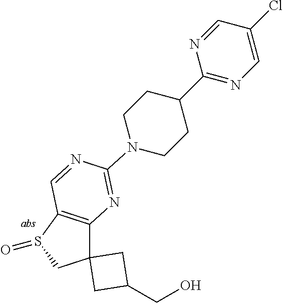

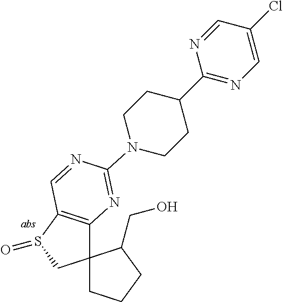

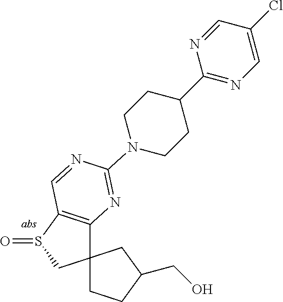

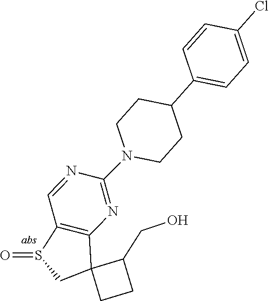

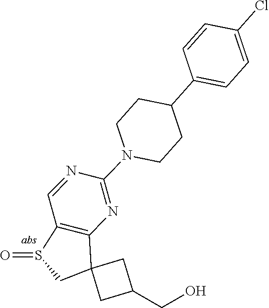

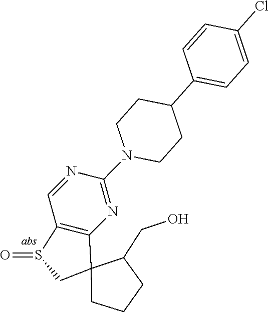

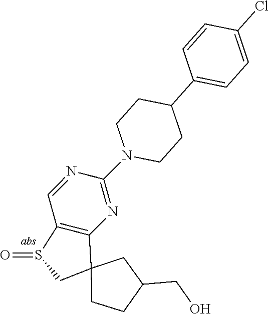

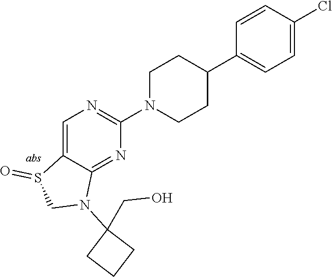 [SMILES: O=[S@]1CN(C2(CO)CCC2)c2nc(N3CCC(c4ccc(Cl)cc4)CC3)ncc21]

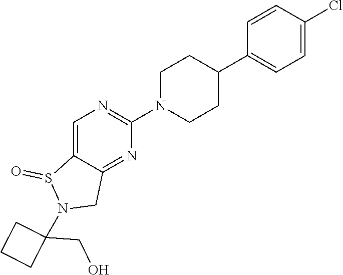 [SMILES: O=S1c2cnc(N3CCC(c4ccc(Cl)cc4)CC3)nc2CN1C1(CO)CCC1]

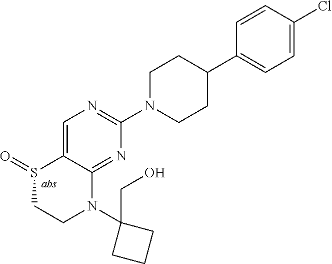 [SMILES: O=[S@]1CCN(C2(CO)CCC2)c2nc(N3CCC(c4ccc(Cl)cc4)CC3)ncc21]

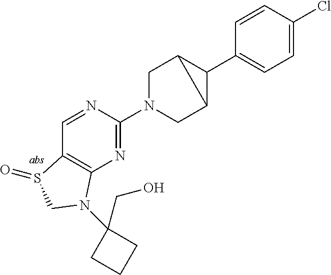 [SMILES: O=[S@]1CN(C2(CO)CCC2)c2nc(N3CC4C(C3)C4c3ccc(Cl)cc3)ncc21]

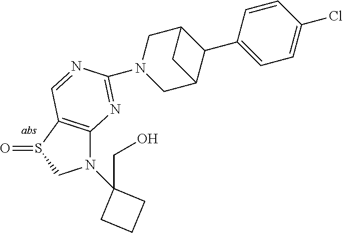 [SMILES: O=[S@]1CN(C2(CO)CCC2)c2nc(N3CC4CC(C3)C4c3ccc(Cl)cc3)ncc21]

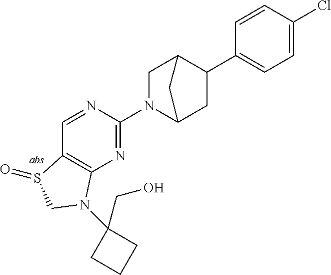 [SMILES: O=[S@]1CN(C2(CO)CCC2)c2nc(N3CC4CC3CC4c3ccc(Cl)cc3)ncc21]

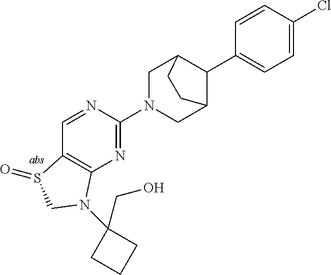 [SMILES: O=[S@]1CN(C2(CO)CCC2)c2nc(N3CC4CCC(C3)C4c3ccc(Cl)cc3)ncc21]

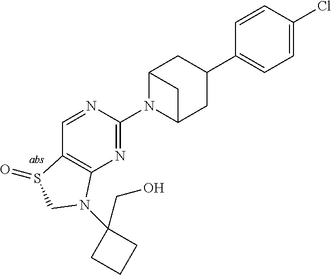 [SMILES: O=[S@]1CN(C2(CO)CCC2)c2nc(N3C4CC(c5ccc(Cl)cc5)CC3C4)ncc21]

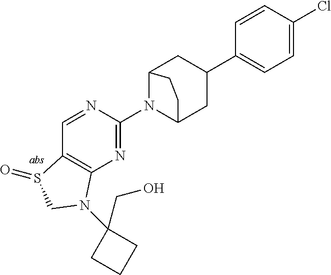 [SMILES: O=[S@]1CN(C2(CO)CCC2)c2nc(N3C4CCC3CC(c3ccc(Cl)cc3)C4)ncc21]

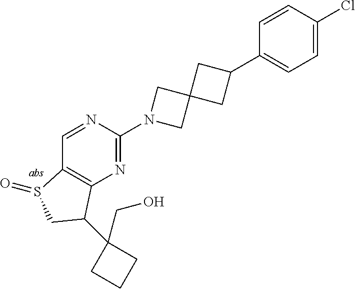

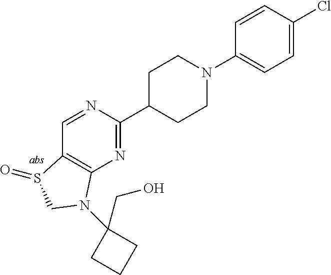 [SMILES: O=[S@]1CN(C2(CO)CCC2)c2nc(C3CCN(c4ccc(Cl)cc4)CC3)ncc21]

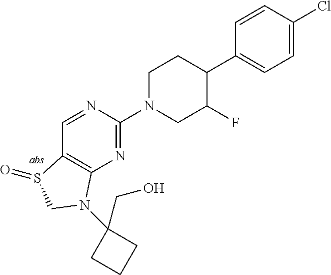 [SMILES: O=[S@]1CN(C2(CO)CCC2)c2nc(N3CCC(c4ccc(Cl)cc4)C(F)C3)ncc21]

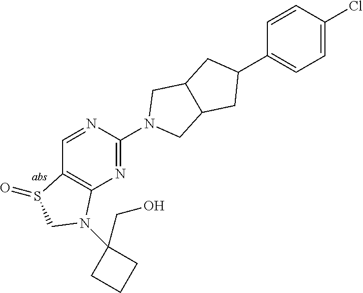 [SMILES: O=[S@]1CN(C2(CO)CCC2)c2nc(N3CC4CC(c5ccc(Cl)cc5)CC4C3)ncc21]

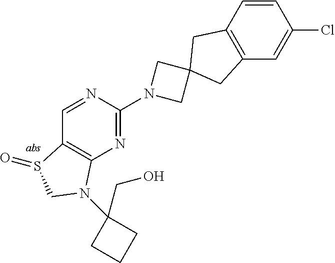 [SMILES: O=[S@]1CN(C2(CO)CCC2)c2nc(N3CC4(Cc5ccc(Cl)cc5C4)C3)ncc21]

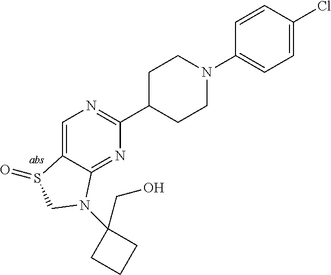 [SMILES: O=[S@]1CN(C2(CO)CCC2)c2nc(C3CCN(c4ccc(Cl)cc4)CC3)ncc21]

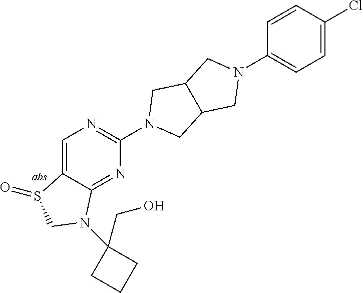 [SMILES: O=[S@]1CN(C2(CO)CCC2)c2nc(N3CC4CN(c5ccc(Cl)cc5)CC4C3)ncc21]

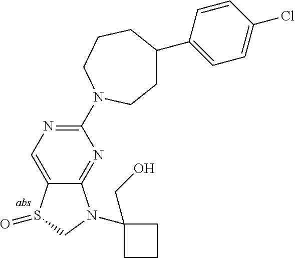 [SMILES: O=[S@]1CN(C2(CO)CCC2)c2nc(N3CCCC(c4ccc(Cl)cc4)CC3)ncc21]

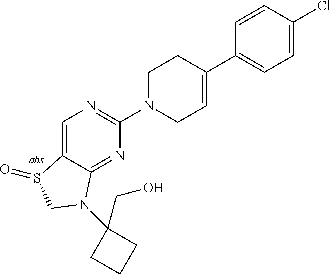 [SMILES: O=[S@]1CN(C2(CO)CCC2)c2nc(N3CC=C(c4ccc(Cl)cc4)CC3)ncc21]

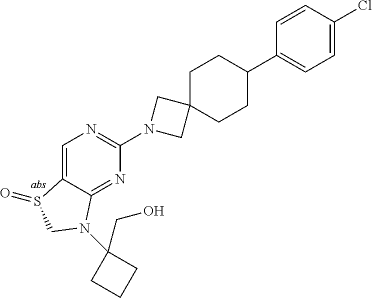 [SMILES: O=[S@]1CN(C2(CO)CCC2)c2nc(N3CC4(CCC(c5ccc(Cl)cc5)CC4)C3)ncc21]

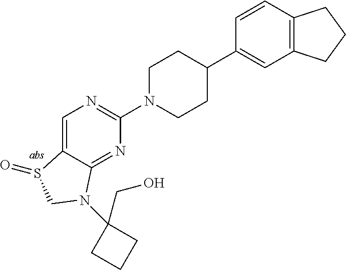 [SMILES: O=[S@]1CN(C2(CO)CCC2)c2nc(N3CCC(c4ccc5c(c4)CCC5)CC3)ncc21]

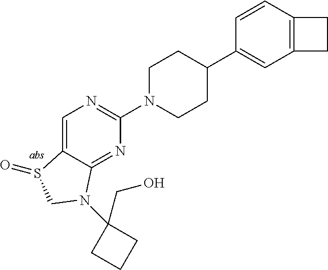 [SMILES: O=[S@]1CN(C2(CO)CCC2)c2nc(N3CCC(c4ccc5c(c4)CC5)CC3)ncc21]

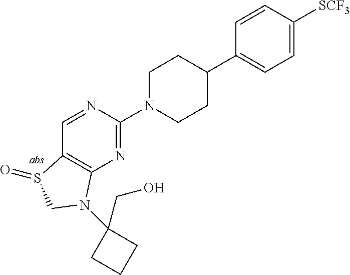

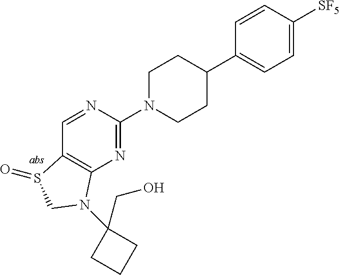

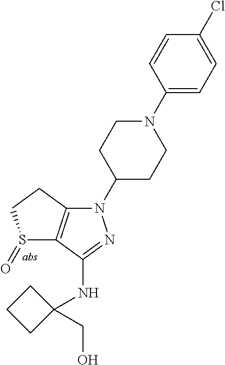 [SMILES: O=[S@@]1CCc2c1c(NC1(CO)CCC1)nn2C1CCN(c2ccc(Cl)cc2)CC1]

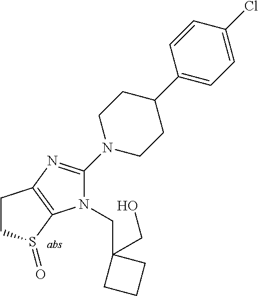 [SMILES: O=[S@@]1CCc2nc(N3CCC(c4ccc(Cl)cc4)CC3)n(CC3(CO)CCC3)c21]

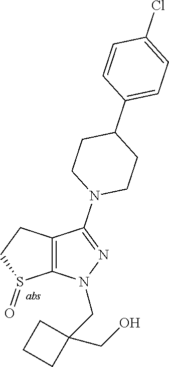 [SMILES: O=[S@@]1CCc2c(N3CCC(c4ccc(Cl)cc4)CC3)nn(CC3(CO)CCC3)c21]

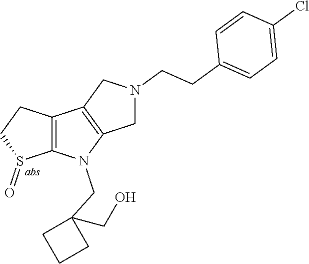 [SMILES: O=[S@@]1CCc2c3c(n(CC4(CO)CCC4)c21)CN(CCc1ccc(Cl)cc1)C3]

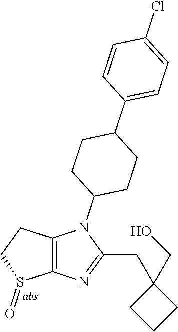 [SMILES: O=[S@@]1CCc2c1nc(CC1(CO)CCC1)n2C1CCC(c2ccc(Cl)cc2)CC1]

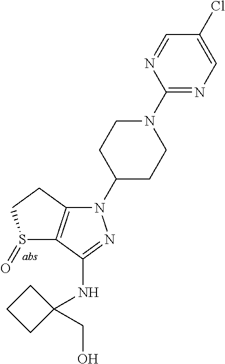 [SMILES: O=[S@@]1CCc2c1c(NC1(CO)CCC1)nn2C1CCN(c2ncc(Cl)cn2)CC1]

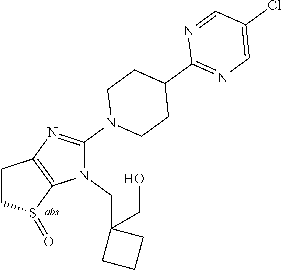 [SMILES: O=[S@@]1CCc2nc(N3CCC(c4ncc(Cl)cn4)CC3)n(CC3(CO)CCC3)c21]

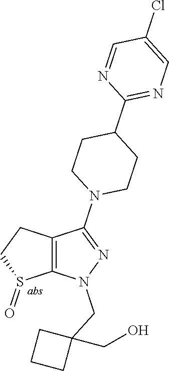 [SMILES: O=[S@@]1CCc2c(N3CCC(c4ncc(Cl)cn4)CC3)nn(CC3(CO)CCC3)c21]

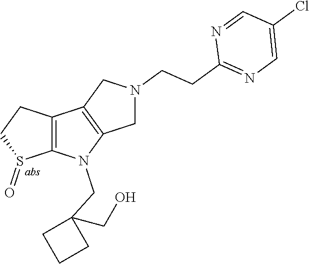 [SMILES: O=[S@@]1CCc2c3c(n(CC4(CO)CCC4)c21)CN(CCc1ncc(Cl)cn1)C3]

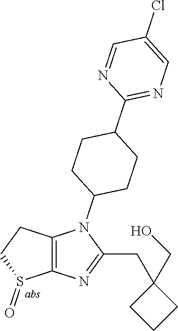 [SMILES: O=[S@@]1CCc2c1nc(CC1(CO)CCC1)n2C1CCC(c2ncc(Cl)cn2)CC1]

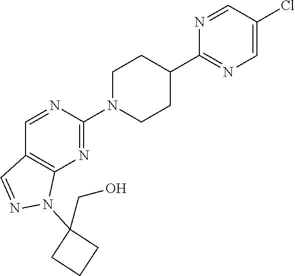 [SMILES: OCC1(n2ncc3cnc(N4CCC(c5ncc(Cl)cn5)CC4)nc32)CCC1]

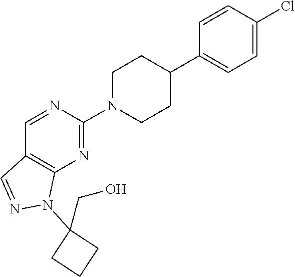 [SMILES: OCC1(n2ncc3cnc(N4CCC(c5ccc(Cl)cc5)CC4)nc32)CCC1]

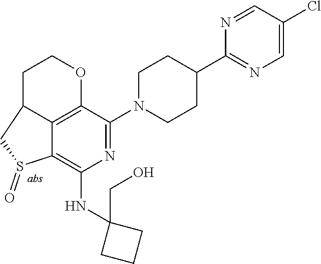

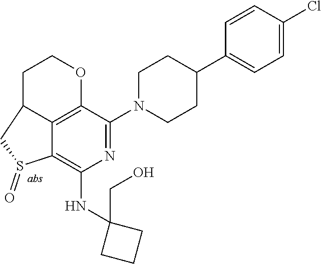

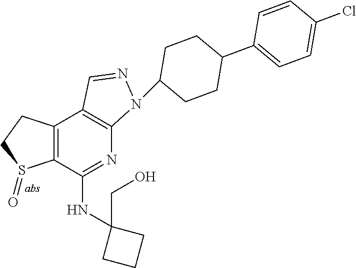 [SMILES: O=[S@]1CCc2c1c(NC1(CO)CCC1)nc1c2cnn1C1CCC(c2ccc(Cl)cc2)CC1]

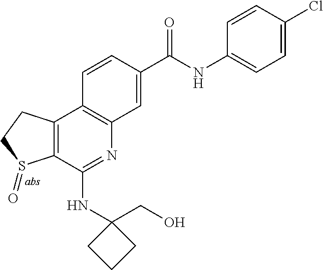 [SMILES: O=C(Nc1ccc(Cl)cc1)c1ccc2c3c(c(NC4(CO)CCC4)nc2c1)[S@@](=O)CC3]

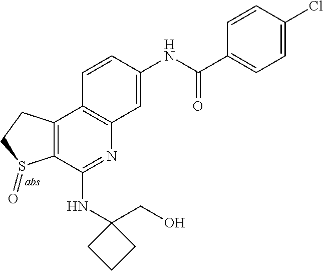 [SMILES: O=C(Nc1ccc2c3c(c(NC4(CO)CCC4)nc2c1)[S@@](=O)CC3)c1ccc(Cl)cc1]

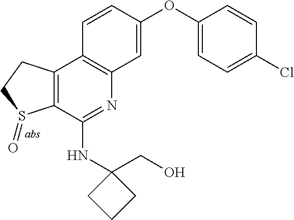 [SMILES: O=[S@]1CCc2c1c(NC1(CO)CCC1)nc1cc(Oc3ccc(Cl)cc3)ccc21]

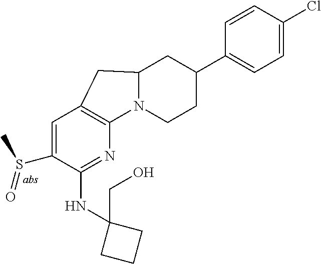 [SMILES: C[S@](=O)c1cc2c(nc1NC1(CO)CCC1)N1CCC(c3ccc(Cl)cc3)CC1C2]

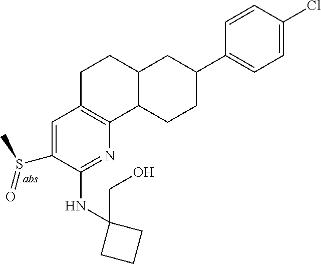 [SMILES: C[S@](=O)c1cc2c(nc1NC1(CO)CCC1)C1CCC(c3ccc(Cl)cc3)CC1CC2]

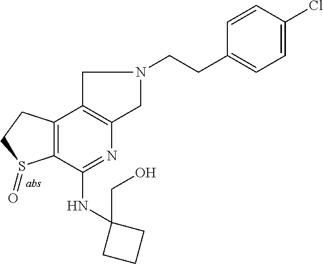 [SMILES: O=[S@]1CCc2c3c(nc(NC4(CO)CCC4)c21)CN(CCc1ccc(Cl)cc1)C3]

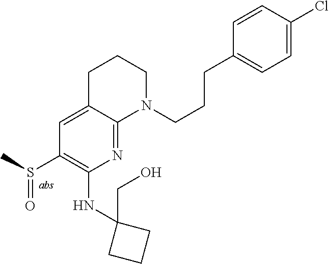 [SMILES: C[S@](=O)c1cc2c(nc1NC1(CO)CCC1)N(CCCc1ccc(Cl)cc1)CCC2]

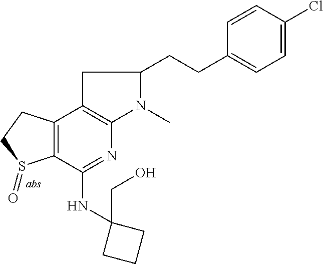 [SMILES: CN1c2nc(NC3(CO)CCC3)c3c(c2CC1CCc1ccc(Cl)cc1)CC[S@@]3=O]

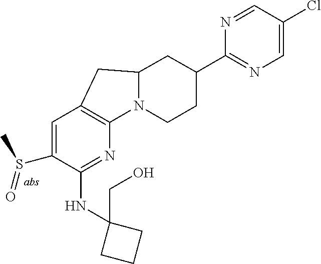 [SMILES: C[S@](=O)c1cc2c(nc1NC1(CO)CCC1)N1CCC(c3ncc(Cl)cn3)CC1C2]

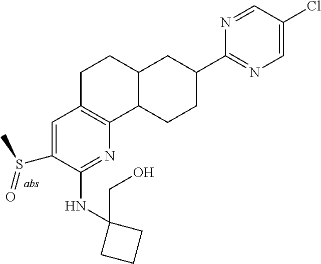 [SMILES: C[S@](=O)c1cc2c(nc1NC1(CO)CCC1)C1CCC(c3ncc(Cl)cn3)CC1CC2]

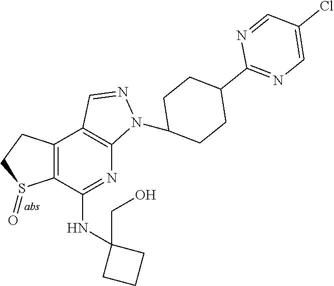 [SMILES: O=[S@]1CCc2c1c(NC1(CO)CCC1)nc1c2cnn1C1CCC(c2ncc(Cl)cn2)CC1]

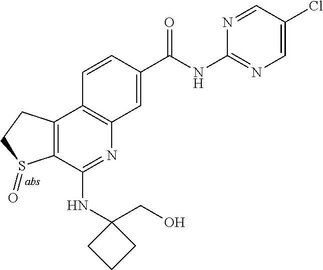 [SMILES: O=C(Nc1ncc(Cl)cn1)c1ccc2c3c(c(NC4(CO)CCC4)nc2c1)[S@@](=O)CC3]

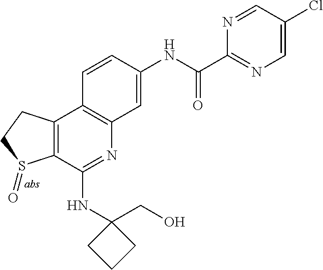 [SMILES: O=C(Nc1ccc2c3c(c(NC4(CO)CCC4)nc2c1)[S@@](=O)CC3)c1ncc(Cl)cn1]

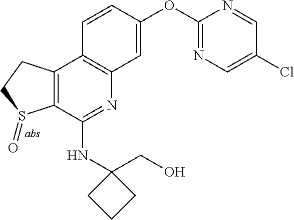 [SMILES: O=[S@]1CCc2c1c(NC1(CO)CCC1)nc1cc(Oc3ncc(Cl)cn3)ccc21]

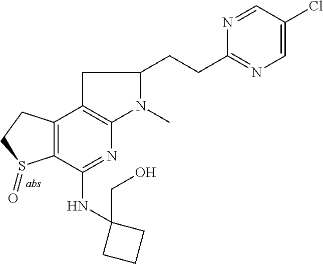 [SMILES: CN1c2nc(NC3(CO)CCC3)c3c(c2CC1CCc1ncc(Cl)cn1)CC[S@@]3=O]

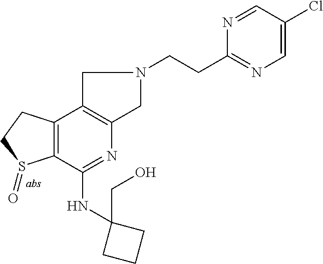 [SMILES: O=[S@]1CCc2c3c(nc(NC4(CO)CCC4)c21)CN(CCc1ncc(Cl)cn1)C3]

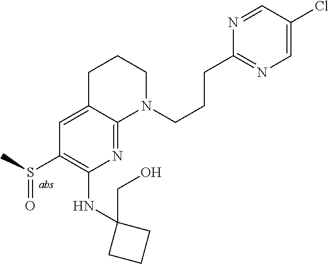 [SMILES: C[S@](=O)c1cc2c(nc1NC1(CO)CCC1)N(CCCc1ncc(Cl)cn1)CCC2]

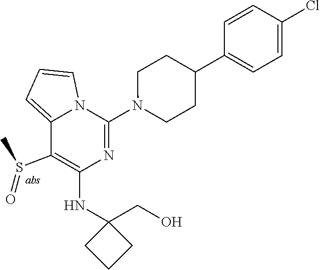 [SMILES: C[S@](=O)c1c(NC2(CO)CCC2)nc(N2CCC(c3ccc(Cl)cc3)CC2)n2cccc12]

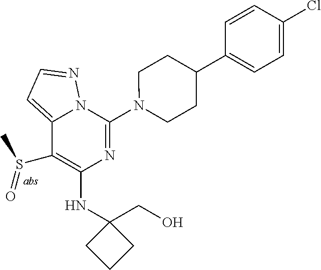 [SMILES: C[S@](=O)c1c(NC2(CO)CCC2)nc(N2CCC(c3ccc(Cl)cc3)CC2)n2nccc12]

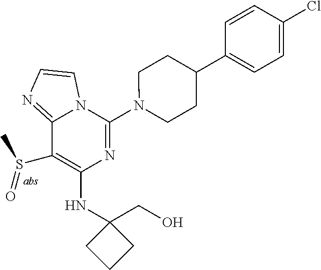 [SMILES: C[S@](=O)c1c(NC2(CO)CCC2)nc(N2CCC(c3ccc(Cl)cc3)CC2)n2ccnc12]

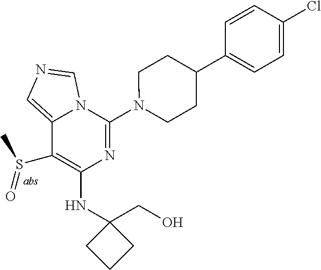 [SMILES: C[S@](=O)c1c(NC2(CO)CCC2)nc(N2CCC(c3ccc(Cl)cc3)CC2)n2cncc12]

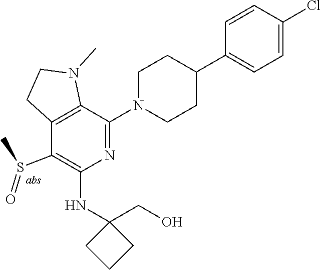 [SMILES: CN1CCc2c1c(N1CCC(c3ccc(Cl)cc3)CC1)nc(NC1(CO)CCC1)c2[S@](C)=O]

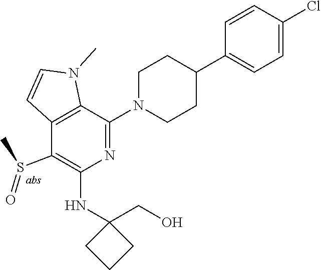 [SMILES: Cn1ccc2c([S@](C)=O)c(NC3(CO)CCC3)nc(N3CCC(c4ccc(Cl)cc4)CC3)c21]

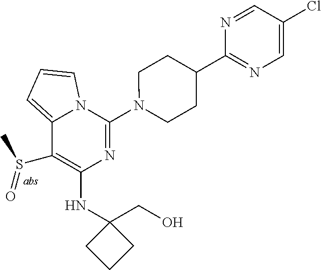 [SMILES: C[S@](=O)c1c(NC2(CO)CCC2)nc(N2CCC(c3ncc(Cl)cn3)CC2)n2cccc12]

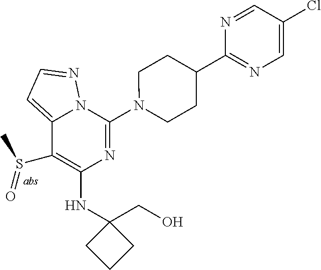 [SMILES: C[S@](=O)c1c(NC2(CO)CCC2)nc(N2CCC(c3ncc(Cl)cn3)CC2)n2nccc12]

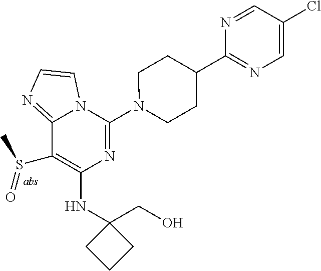 [SMILES: C[S@](=O)c1c(NC2(CO)CCC2)nc(N2CCC(c3ncc(Cl)cn3)CC2)n2ccnc12]

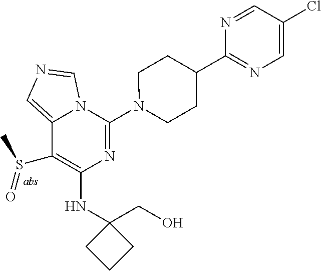 [SMILES: C[S@](=O)c1c(NC2(CO)CCC2)nc(N2CCC(c3ncc(Cl)cn3)CC2)n2cncc12]

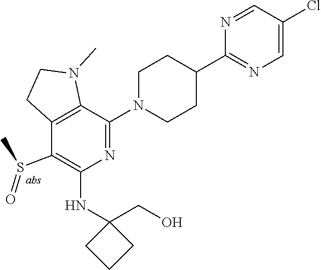 [SMILES: CN1CCc2c1c(N1CCC(c3ncc(Cl)cn3)CC1)nc(NC1(CO)CCC1)c2[S@](C)=O]

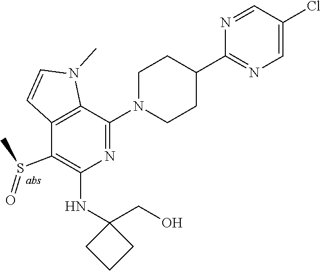 [SMILES: Cn1ccc2c([S@](C)=O)c(NC3(CO)CCC3)nc(N3CCC(c4ncc(Cl)cn4)CC3)c21]

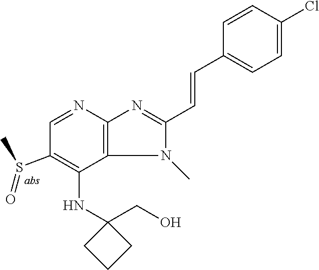 [SMILES: Cn1c(/C=C/c2ccc(Cl)cc2)nc2ncc([S@](C)=O)c(NC3(CO)CCC3)c21]

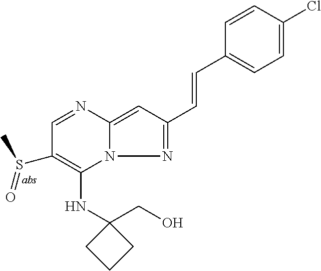 [SMILES: C[S@](=O)c1cnc2cc(/C=C/c3ccc(Cl)cc3)nn2c1NC1(CO)CCC1]

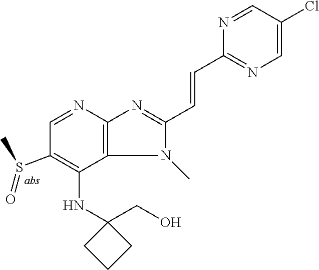 [SMILES: Cn1c(/C=C/c2ncc(Cl)cn2)nc2ncc([S@](C)=O)c(NC3(CO)CCC3)c21]

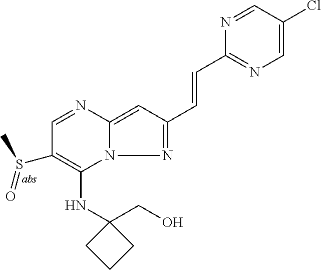 [SMILES: C[S@](=O)c1cnc2cc(/C=C/c3ncc(Cl)cn3)nn2c1NC1(CO)CCC1]

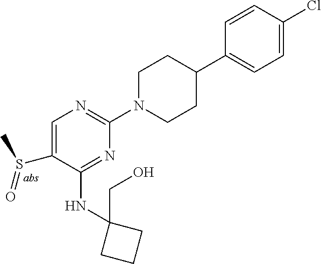 [SMILES: C[S@](=O)c1cnc(N2CCC(c3ccc(Cl)cc3)CC2)nc1NC1(CO)CCC1]

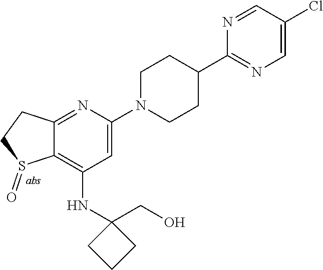 [SMILES: O=[S@]1CCc2nc(N3CCC(c4ncc(Cl)cn4)CC3)cc(NC3(CO)CCC3)c21]

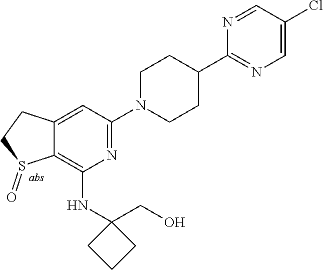 [SMILES: O=[S@]1CCc2cc(N3CCC(c4ncc(Cl)cn4)CC3)nc(NC3(CO)CCC3)c21]

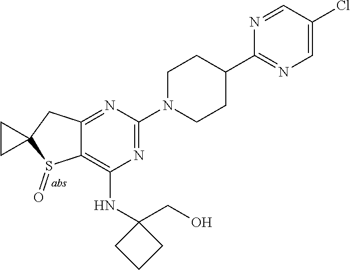 [SMILES: O=[S@@]1c2c(nc(N3CCC(c4ncc(Cl)cn4)CC3)nc2NC2(CO)CCC2)CC12CC2]

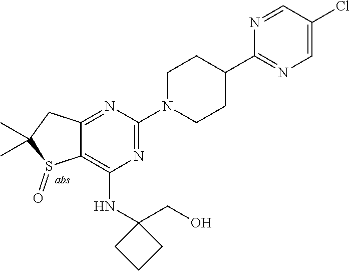 [SMILES: CC1(C)Cc2nc(N3CCC(c4ncc(Cl)cn4)CC3)nc(NC3(CO)CCC3)c2[S@]1=O]

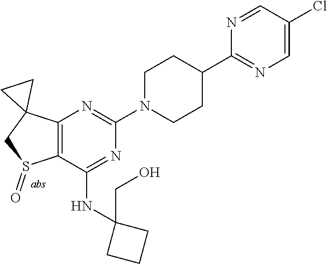 [SMILES: O=[S@]1CC2(CC2)c2nc(N3CCC(c4ncc(Cl)cn4)CC3)nc(NC3(CO)CCC3)c21]

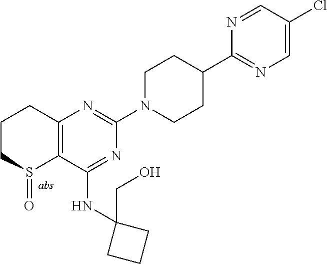 [SMILES: O=[S@]1CCCc2nc(N3CCC(c4ncc(Cl)cn4)CC3)nc(NC3(CO)CCC3)c21]

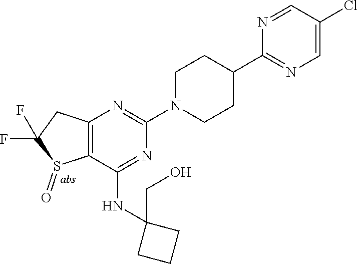 [SMILES: O=[S@@]1c2c(nc(N3CCC(c4ncc(Cl)cn4)CC3)nc2NC2(CO)CCC2)CC1(F)F]

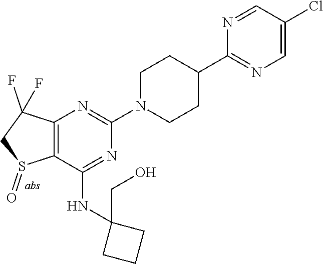 [SMILES: O=[S@]1CC(F)(F)c2nc(N3CCC(c4ncc(Cl)cn4)CC3)nc(NC3(CO)CCC3)c21]

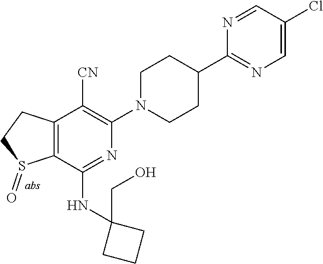 [SMILES: N#Cc1c(N2CCC(c3ncc(Cl)cn3)CC2)nc(NC2(CO)CCC2)c2c1CC[S@@]2=O]

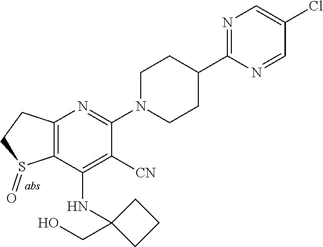 [SMILES: N#Cc1c(N2CCC(c3ncc(Cl)cn3)CC2)nc2c(c1NC1(CO)CCC1)[S@@](=O)CC2]

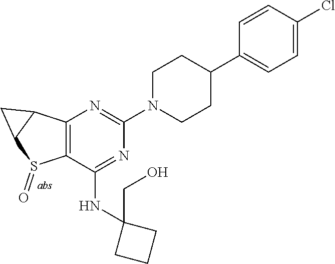

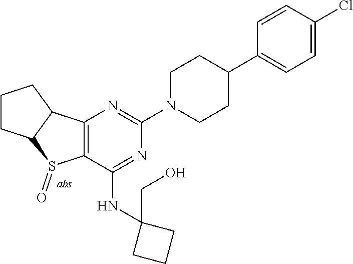

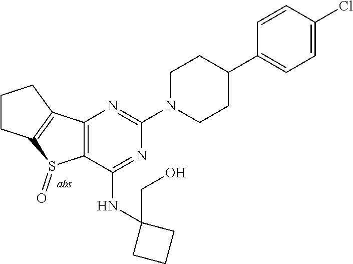 [SMILES: O=[S@]1C2=C(CCC2)c2nc(N3CCC(c4ccc(Cl)cc4)CC3)nc(NC3(CO)CCC3)c21]

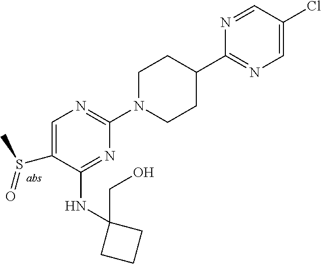 [SMILES: C[S@](=O)c1cnc(N2CCC(c3ncc(Cl)cn3)CC2)nc1NC1(CO)CCC1]

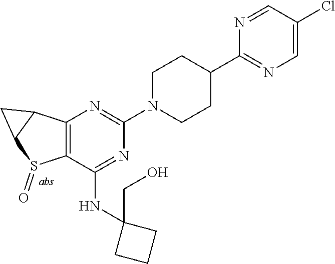

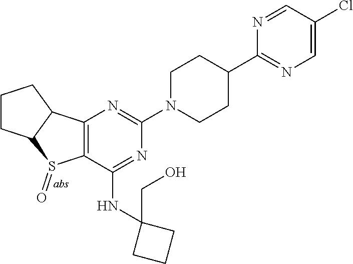

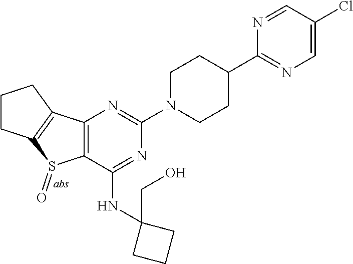 [SMILES: O=[S@]1C2=C(CCC2)c2nc(N3CCC(c4ncc(Cl)cn4)CC3)nc(NC3(CO)CCC3)c21]

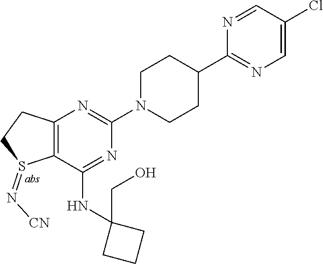 [SMILES: N#C/N=[S@]1/CCc2nc(N3CCC(c4ncc(Cl)cn4)CC3)nc(NC3(CO)CCC3)c21]

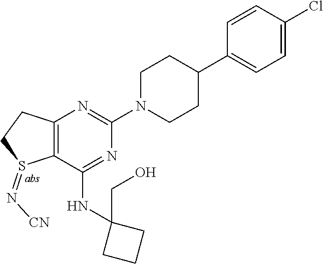 [SMILES: N#C/N=[S@]1/CCc2nc(N3CCC(c4ccc(Cl)cc4)CC3)nc(NC3(CO)CCC3)c21]

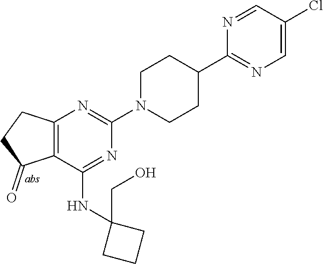

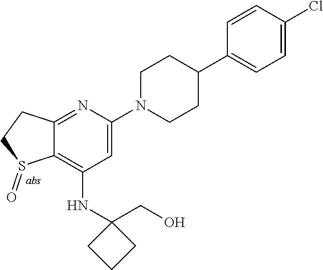 [SMILES: O=[S@]1CCc2nc(N3CCC(c4ccc(Cl)cc4)CC3)cc(NC3(CO)CCC3)c21]

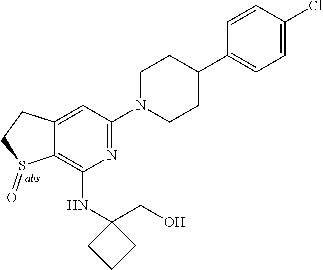 [SMILES: O=[S@]1CCc2cc(N3CCC(c4ccc(Cl)cc4)CC3)nc(NC3(CO)CCC3)c21]

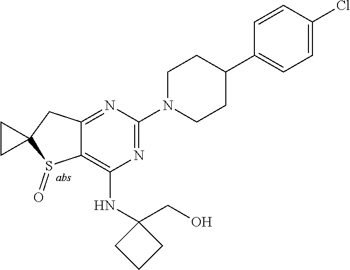 [SMILES: O=[S@@]1c2c(nc(N3CCC(c4ccc(Cl)cc4)CC3)nc2NC2(CO)CCC2)CC12CC2]

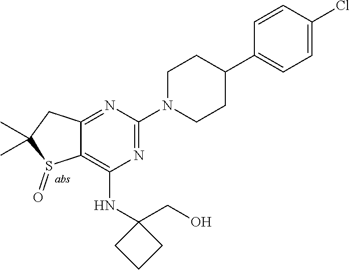 [SMILES: CC1(C)Cc2nc(N3CCC(c4ccc(Cl)cc4)CC3)nc(NC3(CO)CCC3)c2[S@]1=O]

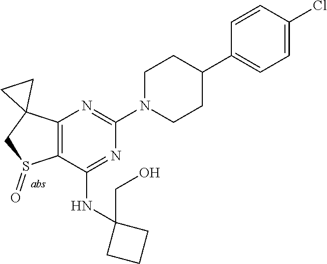 [SMILES: O=[S@]1CC2(CC2)c2nc(N3CCC(c4ccc(Cl)cc4)CC3)nc(NC3(CO)CCC3)c21]

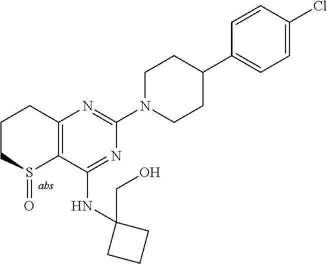 [SMILES: O=[S@]1CCCc2nc(N3CCC(c4ccc(Cl)cc4)CC3)nc(NC3(CO)CCC3)c21]

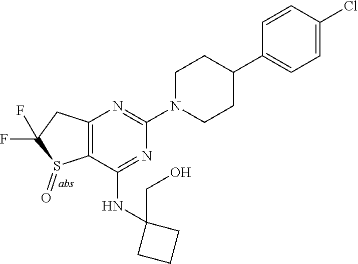 [SMILES: O=[S@@]1c2c(nc(N3CCC(c4ccc(Cl)cc4)CC3)nc2NC2(CO)CCC2)CC1(F)F]

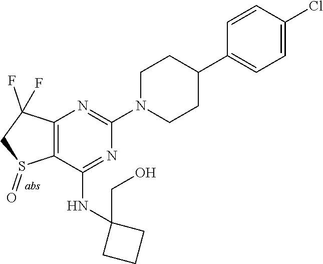 [SMILES: O=[S@]1CC(F)(F)c2nc(N3CCC(c4ccc(Cl)cc4)CC3)nc(NC3(CO)CCC3)c21]

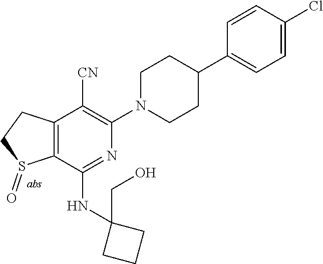 [SMILES: N#Cc1c(N2CCC(c3ccc(Cl)cc3)CC2)nc(NC2(CO)CCC2)c2c1CC[S@@]2=O]

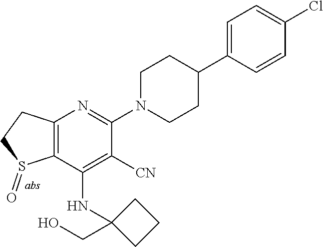 [SMILES: N#Cc1c(N2CCC(c3ccc(Cl)cc3)CC2)nc2c(c1NC1(CO)CCC1)[S@@](=O)CC2]

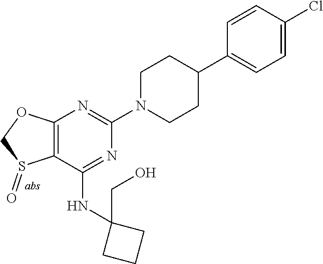 [SMILES: O=[S@]1COc2nc(N3CCC(c4ccc(Cl)cc4)CC3)nc(NC3(CO)CCC3)c21]

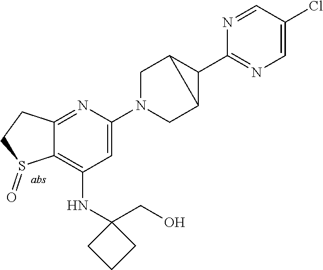 [SMILES: O=[S@]1CCc2nc(N3CC4C(C3)C4c3ncc(Cl)cn3)cc(NC3(CO)CCC3)c21]

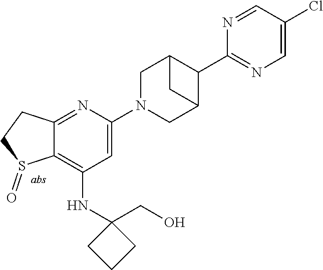 [SMILES: O=[S@]1CCc2nc(N3CC4CC(C3)C4c3ncc(Cl)cn3)cc(NC3(CO)CCC3)c21]

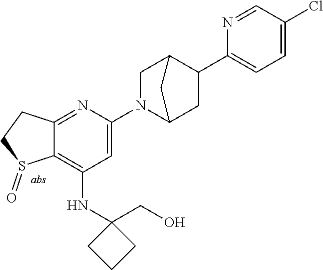 [SMILES: O=[S@]1CCc2nc(N3CC4CC3CC4c3ccc(Cl)cn3)cc(NC3(CO)CCC3)c21]

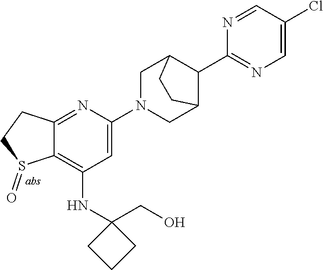 [SMILES: O=[S@]1CCc2nc(N3CC4CCC(C3)C4c3ncc(Cl)cn3)cc(NC3(CO)CCC3)c21]

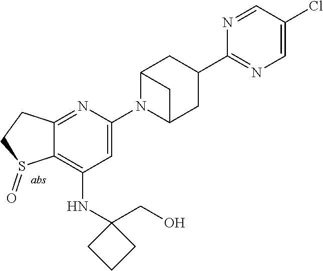 [SMILES: O=[S@]1CCc2nc(N3C4CC(c5ncc(Cl)cn5)CC3C4)cc(NC3(CO)CCC3)c21]

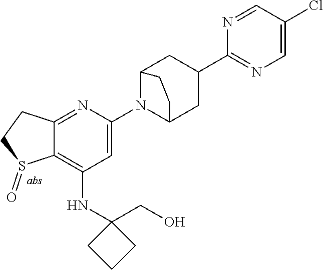 [SMILES: O=[S@]1CCc2nc(N3C4CCC3CC(c3ncc(Cl)cn3)C4)cc(NC3(CO)CCC3)c21]

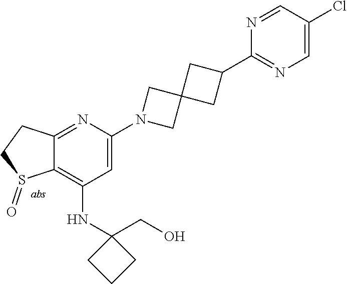 [SMILES: O=[S@]1CCc2nc(N3CC4(CC(c5ncc(Cl)cn5)C4)C3)cc(NC3(CO)CCC3)c21]

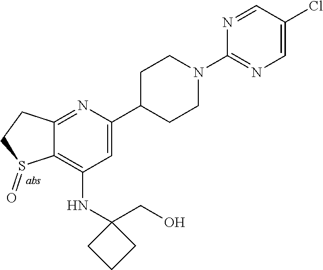 [SMILES: O=[S@]1CCc2nc(C3CCN(c4ncc(Cl)cn4)CC3)cc(NC3(CO)CCC3)c21]

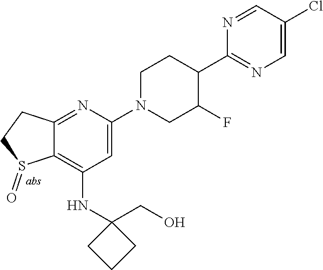 [SMILES: O=[S@]1CCc2nc(N3CCC(c4ncc(Cl)cn4)C(F)C3)cc(NC3(CO)CCC3)c21]

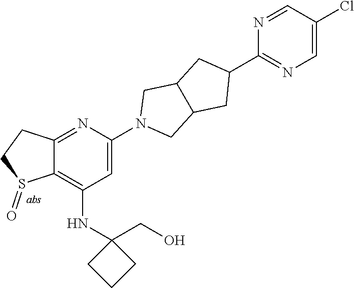 [SMILES: O=[S@]1CCc2nc(N3CC4CC(c5ncc(Cl)cn5)CC4C3)cc(NC3(CO)CCC3)c21]

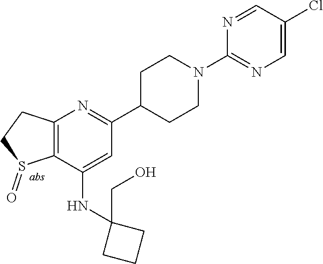 [SMILES: O=[S@]1CCc2nc(C3CCN(c4ncc(Cl)cn4)CC3)cc(NC3(CO)CCC3)c21]

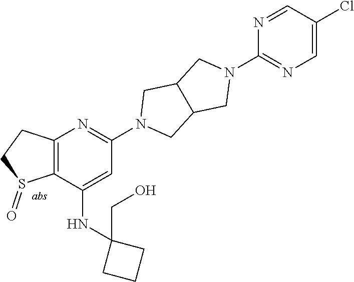 [SMILES: O=[S@]1CCc2nc(N3CC4CN(c5ncc(Cl)cn5)CC4C3)cc(NC3(CO)CCC3)c21]

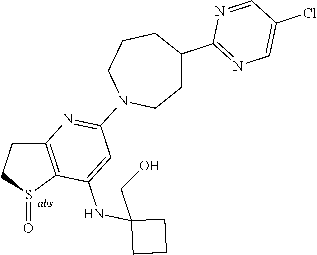 [SMILES: O=[S@]1CCc2nc(N3CCCC(c4ncc(Cl)cn4)CC3)cc(NC3(CO)CCC3)c21]

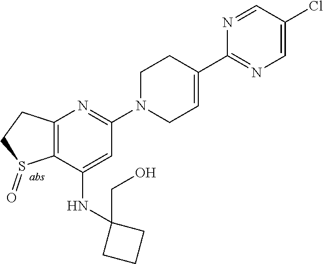 [SMILES: O=[S@]1CCc2nc(N3CC=C(c4ncc(Cl)cn4)CC3)cc(NC3(CO)CCC3)c21]

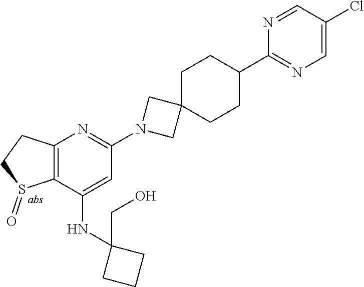 [SMILES: O=[S@]1CCc2nc(N3CC4(CCC(c5ncc(Cl)cn5)CC4)C3)cc(NC3(CO)CCC3)c21]

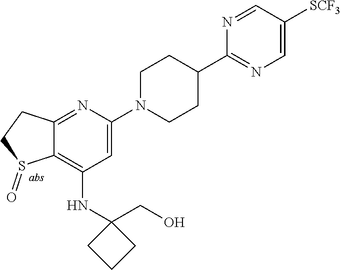

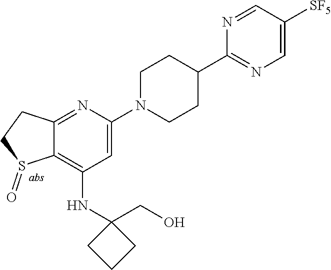

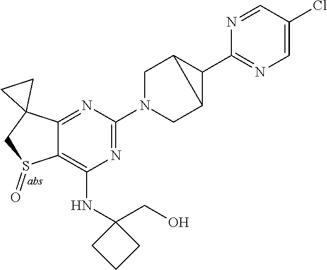 [SMILES: O=[S@]1CC2(CC2)c2nc(N3CC4C(C3)C4c3ncc(Cl)cn3)nc(NC3(CO)CCC3)c21]

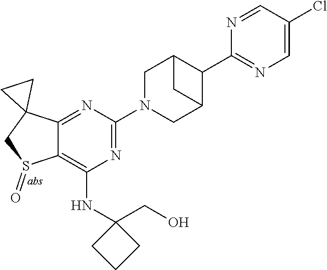 [SMILES: O=[S@]1CC2(CC2)c2nc(N3CC4CC(C3)C4c3ncc(Cl)cn3)nc(NC3(CO)CCC3)c21]

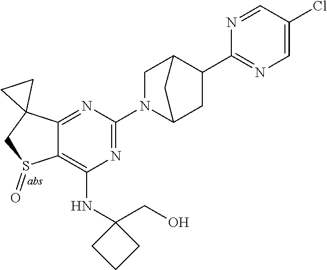 [SMILES: O=[S@]1CC2(CC2)c2nc(N3CC4CC3CC4c3ncc(Cl)cn3)nc(NC3(CO)CCC3)c21]

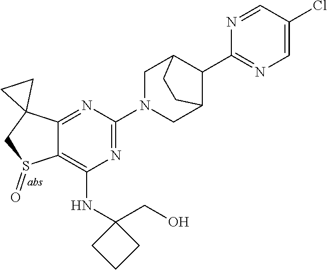 [SMILES: O=[S@]1CC2(CC2)c2nc(N3CC4CCC(C3)C4c3ncc(Cl)cn3)nc(NC3(CO)CCC3)c21]

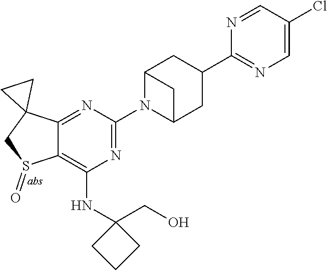 [SMILES: O=[S@]1CC2(CC2)c2nc(N3C4CC(c5ncc(Cl)cn5)CC3C4)nc(NC3(CO)CCC3)c21]

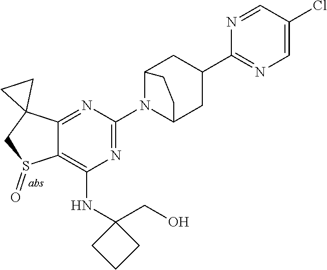 [SMILES: O=[S@]1CC2(CC2)c2nc(N3C4CCC3CC(c3ncc(Cl)cn3)C4)nc(NC3(CO)CCC3)c21]

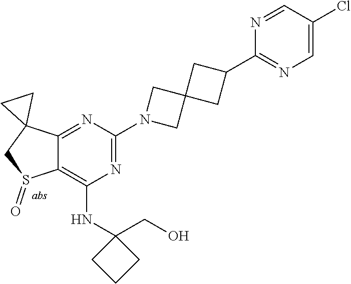 [SMILES: O=[S@]1CC2(CC2)c2nc(N3CC4(CC(c5ncc(Cl)cn5)C4)C3)nc(NC3(CO)CCC3)c21]

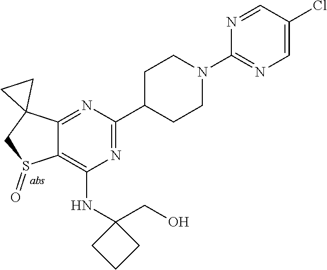 [SMILES: O=[S@]1CC2(CC2)c2nc(C3CCN(c4ncc(Cl)cn4)CC3)nc(NC3(CO)CCC3)c21]

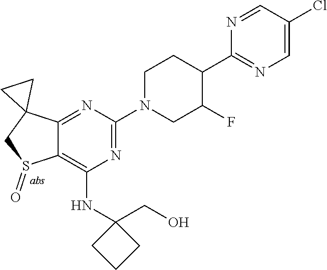 [SMILES: O=[S@]1CC2(CC2)c2nc(N3CCC(c4ncc(Cl)cn4)C(F)C3)nc(NC3(CO)CCC3)c21]

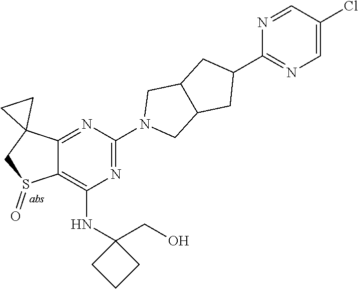 [SMILES: O=[S@]1CC2(CC2)c2nc(N3CC4CC(c5ncc(Cl)cn5)CC4C3)nc(NC3(CO)CCC3)c21]

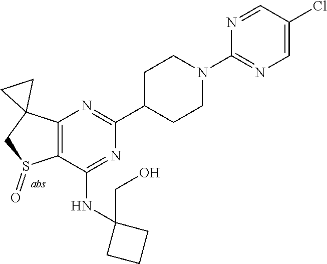 [SMILES: O=[S@]1CC2(CC2)c2nc(C3CCN(c4ncc(Cl)cn4)CC3)nc(NC3(CO)CCC3)c21]

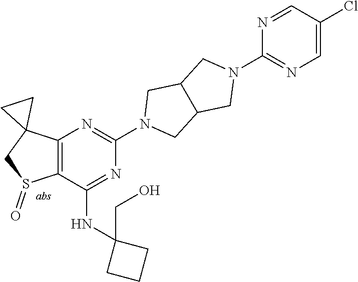 [SMILES: O=[S@]1CC2(CC2)c2nc(N3CC4CN(c5ncc(Cl)cn5)CC4C3)nc(NC3(CO)CCC3)c21]

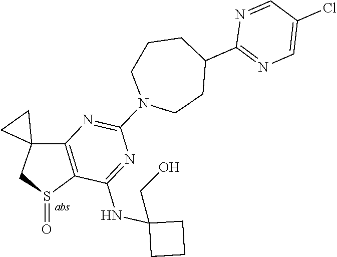 [SMILES: O=[S@]1CC2(CC2)c2nc(N3CCCC(c4ncc(Cl)cn4)CC3)nc(NC3(CO)CCC3)c21]

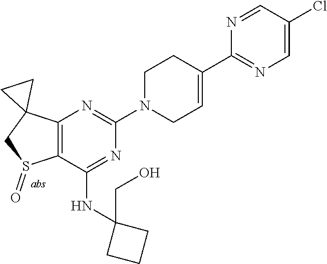 [SMILES: O=[S@]1CC2(CC2)c2nc(N3CC=C(c4ncc(Cl)cn4)CC3)nc(NC3(CO)CCC3)c21]

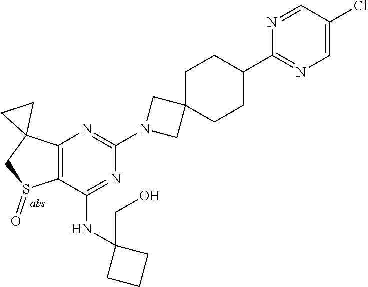 [SMILES: O=[S@]1CC2(CC2)c2nc(N3CC4(CCC(c5ncc(Cl)cn5)CC4)C3)nc(NC3(CO)CCC3)c21]

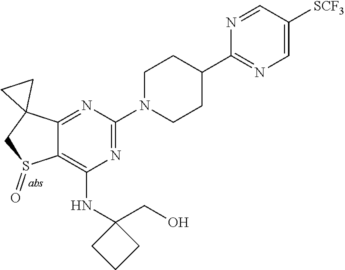

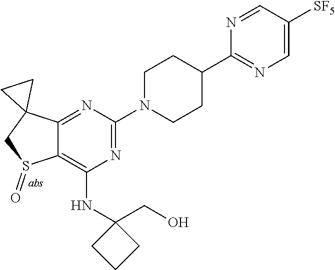

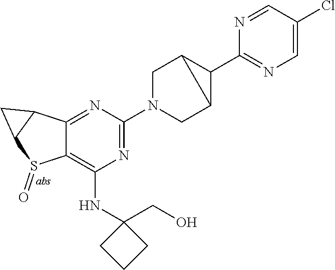

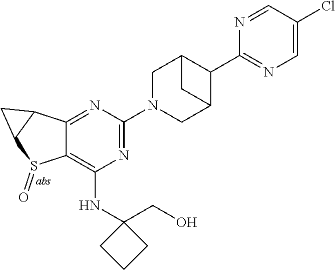

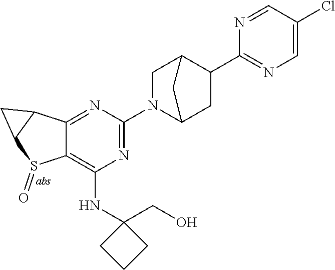

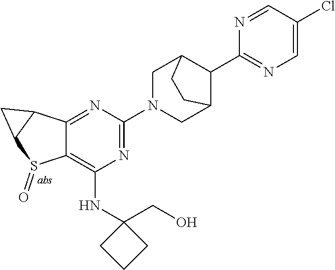

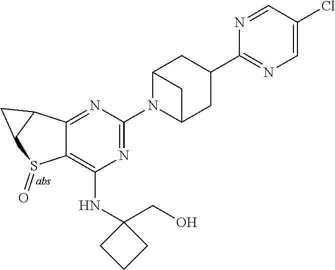

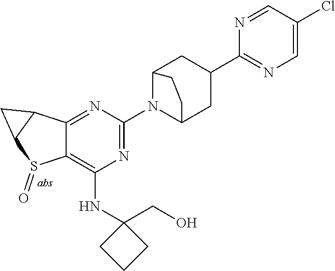

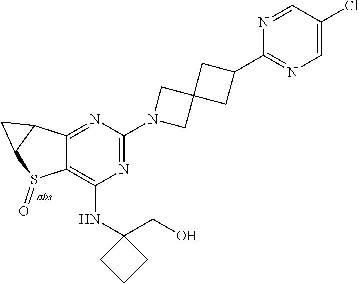

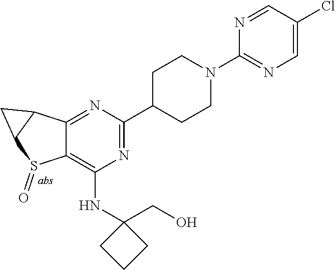

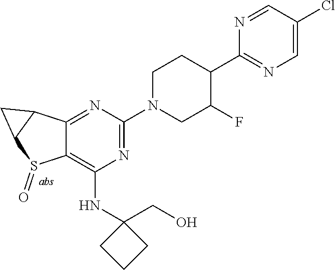

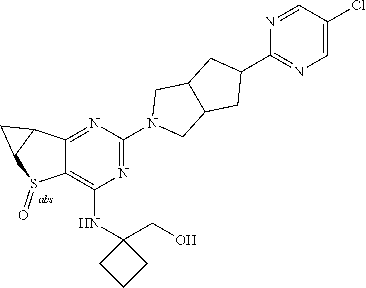

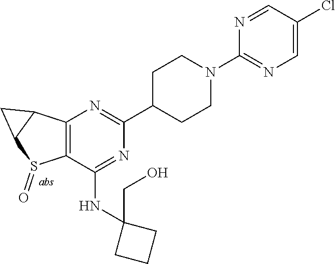

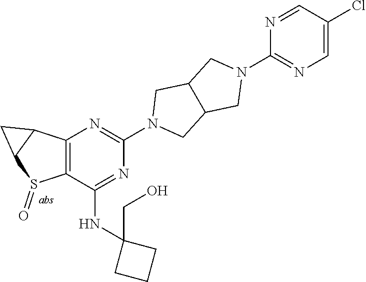

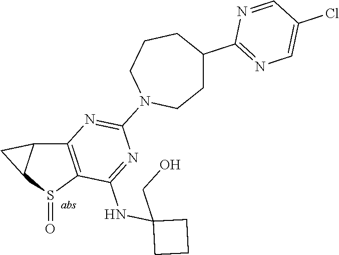

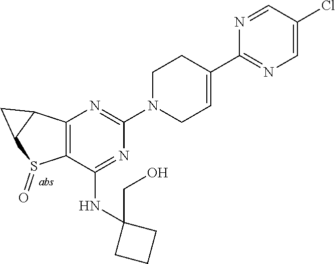

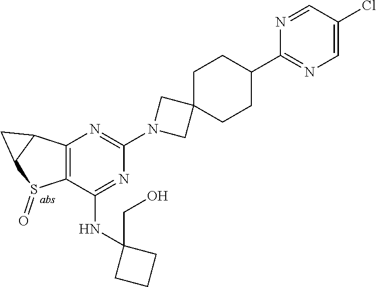

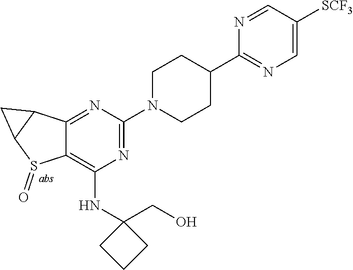

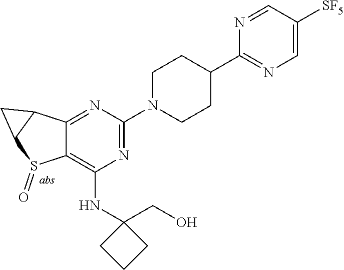

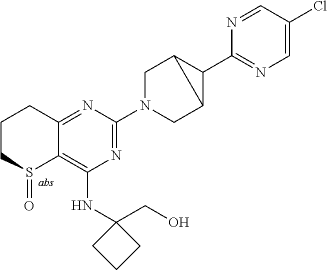 [SMILES: O=[S@]1CCCc2nc(N3CC4C(C3)C4c3ncc(Cl)cn3)nc(NC3(CO)CCC3)c21]

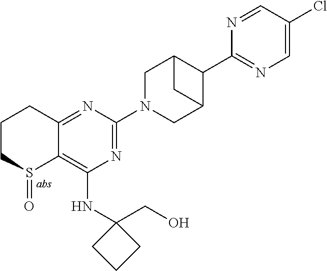 [SMILES: O=[S@]1CCCc2nc(N3CC4CC(C3)C4c3ncc(Cl)cn3)nc(NC3(CO)CCC3)c21]

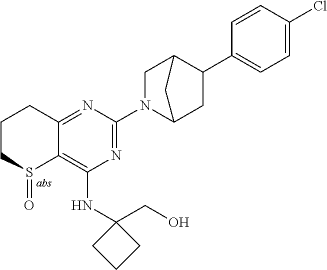 [SMILES: O=[S@]1CCCc2nc(N3CC4CC3CC4c3ccc(Cl)cc3)nc(NC3(CO)CCC3)c21]

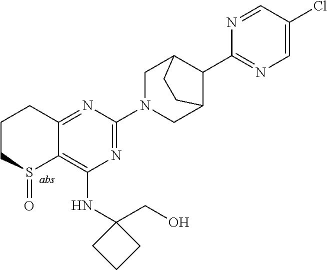 [SMILES: O=[S@]1CCCc2nc(N3CC4CCC(C3)C4c3ncc(Cl)cn3)nc(NC3(CO)CCC3)c21]

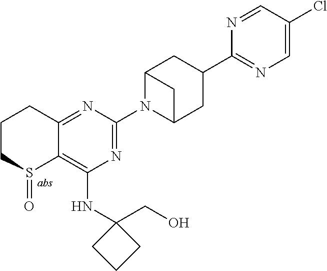 [SMILES: O=[S@]1CCCc2nc(N3C4CC(c5ncc(Cl)cn5)CC3C4)nc(NC3(CO)CCC3)c21]

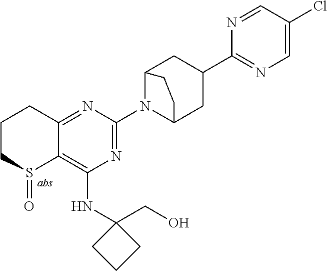 [SMILES: O=[S@]1CCCc2nc(N3C4CCC3CC(c3ncc(Cl)cn3)C4)nc(NC3(CO)CCC3)c21]

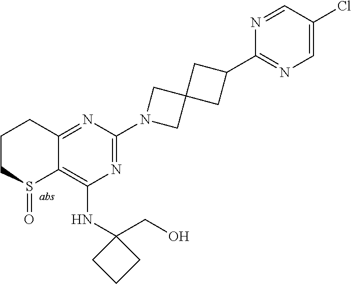 [SMILES: O=[S@]1CCCc2nc(N3CC4(CC(c5ncc(Cl)cn5)C4)C3)nc(NC3(CO)CCC3)c21]

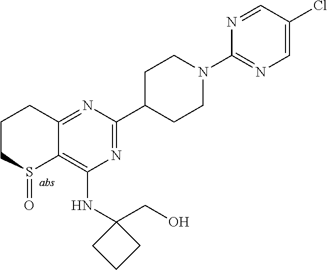 [SMILES: O=[S@]1CCCc2nc(C3CCN(c4ncc(Cl)cn4)CC3)nc(NC3(CO)CCC3)c21]

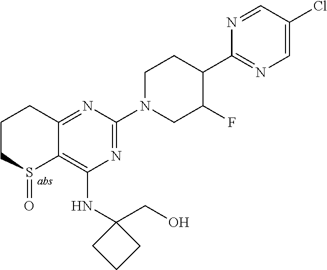 [SMILES: O=[S@]1CCCc2nc(N3CCC(c4ncc(Cl)cn4)C(F)C3)nc(NC3(CO)CCC3)c21]

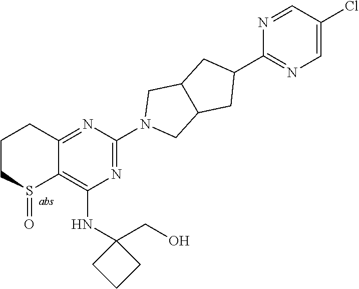 [SMILES: O=[S@]1CCCc2nc(N3CC4CC(c5ncc(Cl)cn5)CC4C3)nc(NC3(CO)CCC3)c21]

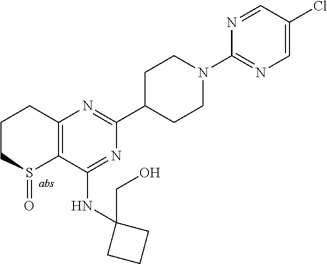 [SMILES: O=[S@]1CCCc2nc(C3CCN(c4ncc(Cl)cn4)CC3)nc(NC3(CO)CCC3)c21]

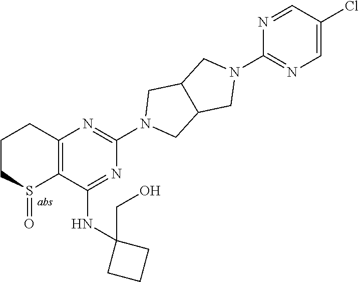 [SMILES: O=[S@]1CCCc2nc(N3CC4CN(c5ncc(Cl)cn5)CC4C3)nc(NC3(CO)CCC3)c21]

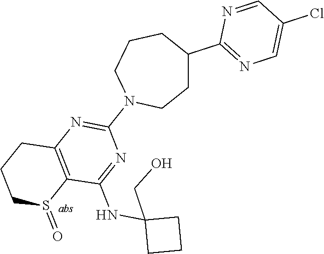 [SMILES: O=[S@]1CCCc2nc(N3CCCC(c4ncc(Cl)cn4)CC3)nc(NC3(CO)CCC3)c21]

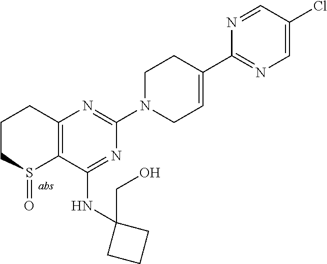 [SMILES: O=[S@]1CCCc2nc(N3CC=C(c4ncc(Cl)cn4)CC3)nc(NC3(CO)CCC3)c21]

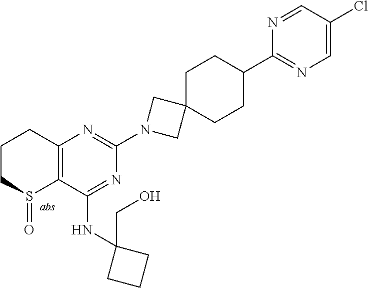 [SMILES: O=[S@]1CCCc2nc(N3CC4(CCC(c5ncc(Cl)cn5)CC4)C3)nc(NC3(CO)CCC3)c21]

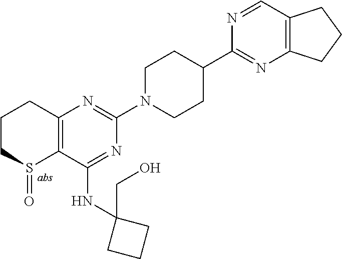 [SMILES: O=[S@]1CCCc2nc(N3CCC(c4ncc5c(n4)CCC5)CC3)nc(NC3(CO)CCC3)c21]

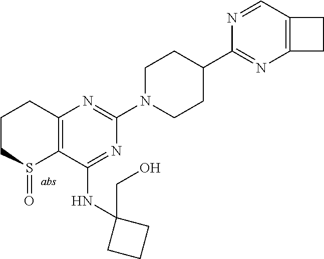 [SMILES: O=[S@]1CCCc2nc(N3CCC(c4ncc5c(n4)CC5)CC3)nc(NC3(CO)CCC3)c21]

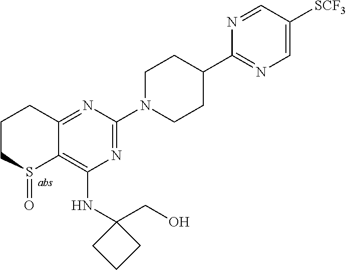

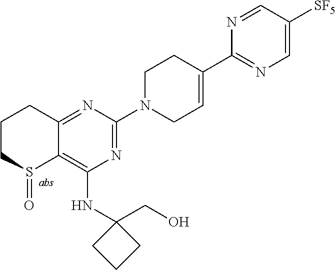

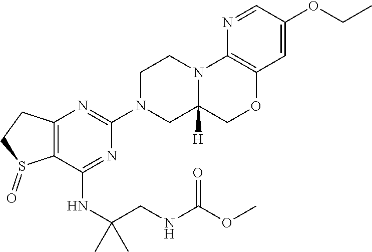 [SMILES: CCOc1cnc2c(c1)OC[C@@H]1CN(c3nc4c(c(NC(C)(C)CNC(=O)OC)n3)[S@@](=O)CC4)CCN21]

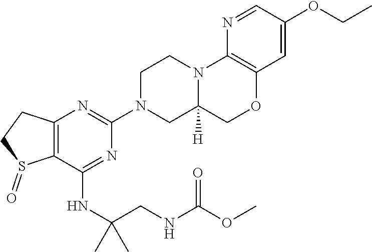 [SMILES: CCOc1cnc2c(c1)OC[C@H]1CN(c3nc4c(c(NC(C)(C)CNC(=O)OC)n3)[S@@](=O)CC4)CCN21]

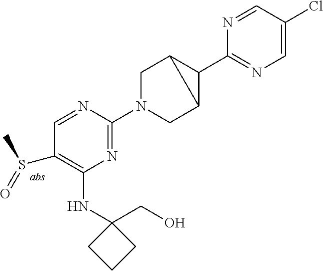 [SMILES: C[S@](=O)c1cnc(N2CC3C(C2)C3c2ncc(Cl)cn2)nc1NC1(CO)CCC1]

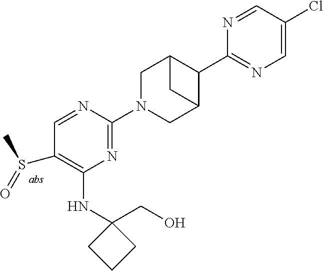 [SMILES: C[S@](=O)c1cnc(N2CC3CC(C2)C3c2ncc(Cl)cn2)nc1NC1(CO)CCC1]

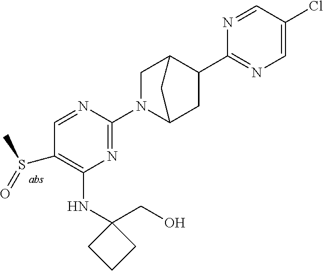 [SMILES: C[S@](=O)c1cnc(N2CC3CC2CC3c2ncc(Cl)cn2)nc1NC1(CO)CCC1]

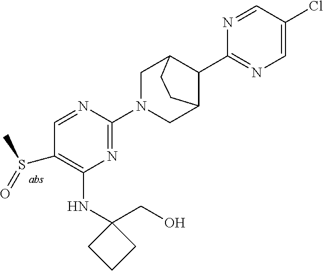 [SMILES: C[S@](=O)c1cnc(N2CC3CCC(C2)C3c2ncc(Cl)cn2)nc1NC1(CO)CCC1]

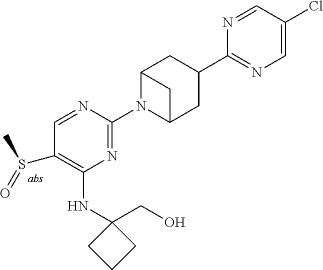 [SMILES: C[S@](=O)c1cnc(N2C3CC(c4ncc(Cl)cn4)CC2C3)nc1NC1(CO)CCC1]

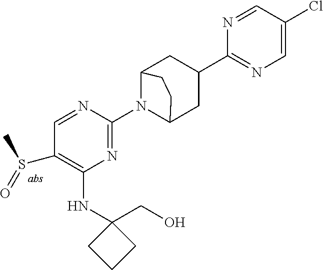 [SMILES: C[S@](=O)c1cnc(N2C3CCC2CC(c2ncc(Cl)cn2)C3)nc1NC1(CO)CCC1]

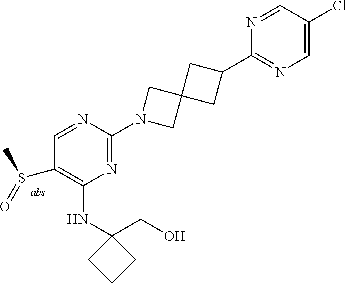 [SMILES: C[S@](=O)c1cnc(N2CC3(CC(c4ncc(Cl)cn4)C3)C2)nc1NC1(CO)CCC1]

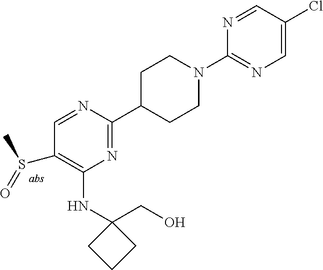 [SMILES: C[S@](=O)c1cnc(C2CCN(c3ncc(Cl)cn3)CC2)nc1NC1(CO)CCC1]

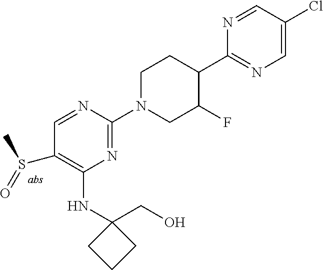 [SMILES: C[S@](=O)c1cnc(N2CCC(c3ncc(Cl)cn3)C(F)C2)nc1NC1(CO)CCC1]

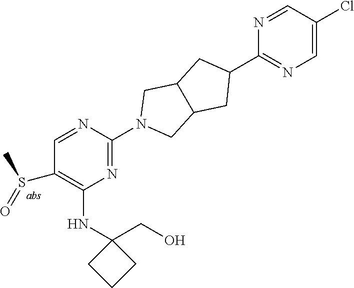 [SMILES: C[S@](=O)c1cnc(N2CC3CC(c4ncc(Cl)cn4)CC3C2)nc1NC1(CO)CCC1]

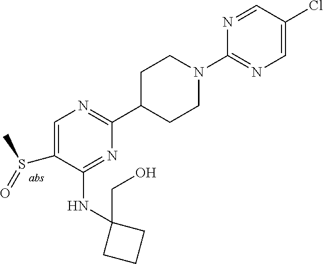 [SMILES: C[S@](=O)c1cnc(C2CCN(c3ncc(Cl)cn3)CC2)nc1NC1(CO)CCC1]

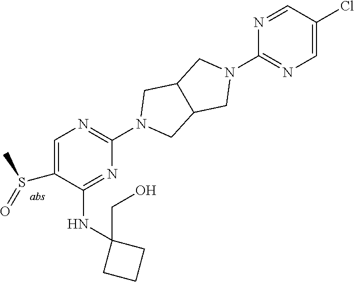 [SMILES: C[S@](=O)c1cnc(N2CC3CN(c4ncc(Cl)cn4)CC3C2)nc1NC1(CO)CCC1]

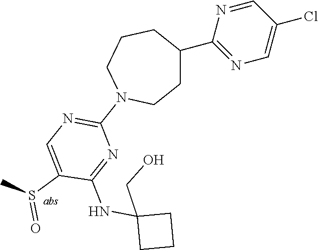 [SMILES: C[S@](=O)c1cnc(N2CCCC(c3ncc(Cl)cn3)CC2)nc1NC1(CO)CCC1]

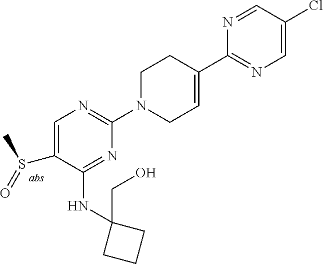 [SMILES: C[S@](=O)c1cnc(N2CC=C(c3ncc(Cl)cn3)CC2)nc1NC1(CO)CCC1]

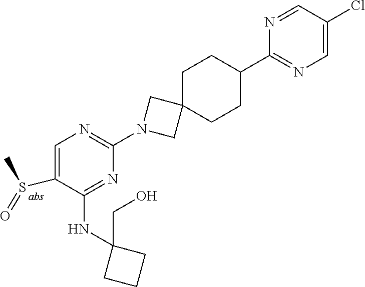 [SMILES: C[S@](=O)c1cnc(N2CC3(CCC(c4ncc(Cl)cn4)CC3)C2)nc1NC1(CO)CCC1]

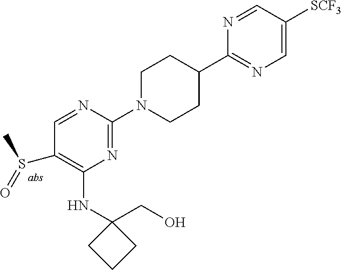

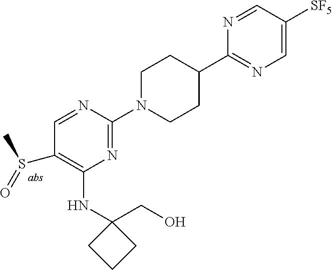

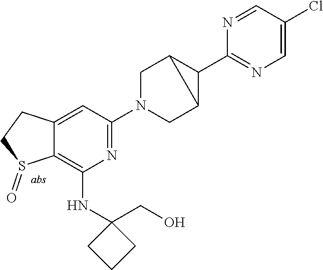 [SMILES: O=[S@]1CCc2cc(N3CC4C(C3)C4c3ncc(Cl)cn3)nc(NC3(CO)CCC3)c21]

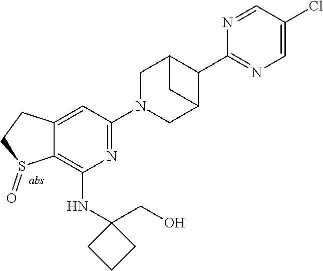 [SMILES: O=[S@]1CCc2cc(N3CC4CC(C3)C4c3ncc(Cl)cn3)nc(NC3(CO)CCC3)c21]

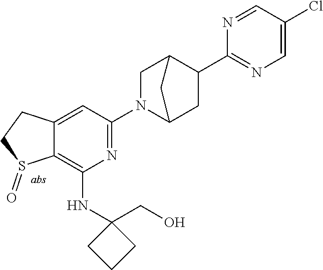 [SMILES: O=[S@]1CCc2cc(N3CC4CC3CC4c3ncc(Cl)cn3)nc(NC3(CO)CCC3)c21]

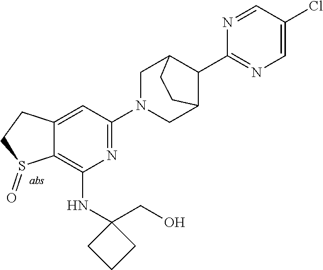 [SMILES: O=[S@]1CCc2cc(N3CC4CCC(C3)C4c3ncc(Cl)cn3)nc(NC3(CO)CCC3)c21]

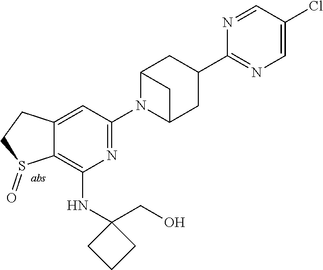 [SMILES: O=[S@]1CCc2cc(N3C4CC(c5ncc(Cl)cn5)CC3C4)nc(NC3(CO)CCC3)c21]

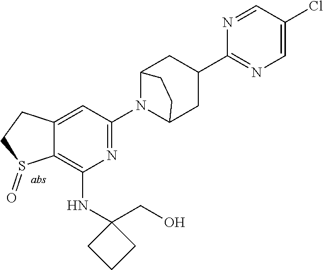 [SMILES: O=[S@]1CCc2cc(N3C4CCC3CC(c3ncc(Cl)cn3)C4)nc(NC3(CO)CCC3)c21]

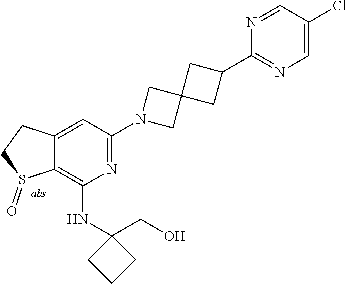 [SMILES: O=[S@]1CCc2cc(N3CC4(CC(c5ncc(Cl)cn5)C4)C3)nc(NC3(CO)CCC3)c21]

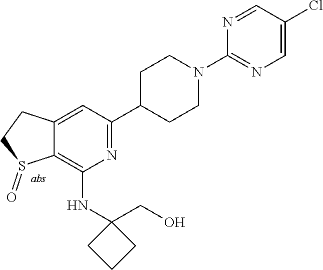 [SMILES: O=[S@]1CCc2cc(C3CCN(c4ncc(Cl)cn4)CC3)nc(NC3(CO)CCC3)c21]

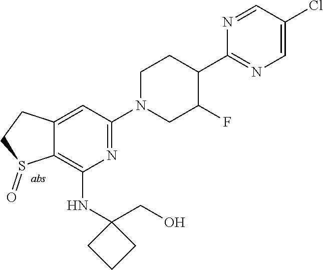 [SMILES: O=[S@]1CCc2cc(N3CCC(c4ncc(Cl)cn4)C(F)C3)nc(NC3(CO)CCC3)c21]

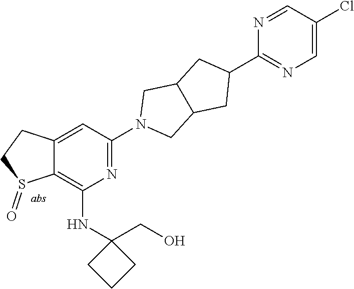 [SMILES: O=[S@]1CCc2cc(N3CC4CC(c5ncc(Cl)cn5)CC4C3)nc(NC3(CO)CCC3)c21]

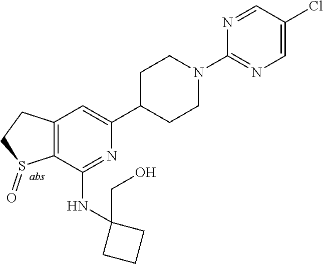 [SMILES: O=[S@]1CCc2cc(C3CCN(c4ncc(Cl)cn4)CC3)nc(NC3(CO)CCC3)c21]

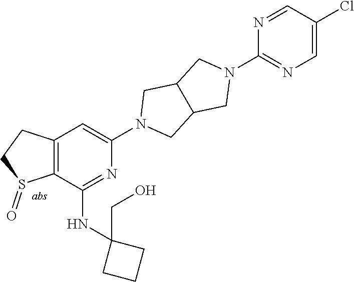 [SMILES: O=[S@]1CCc2cc(N3CC4CN(c5ncc(Cl)cn5)CC4C3)nc(NC3(CO)CCC3)c21]

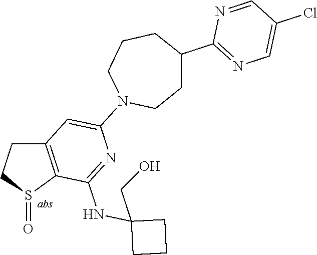 [SMILES: O=[S@]1CCc2cc(N3CCCC(c4ncc(Cl)cn4)CC3)nc(NC3(CO)CCC3)c21]

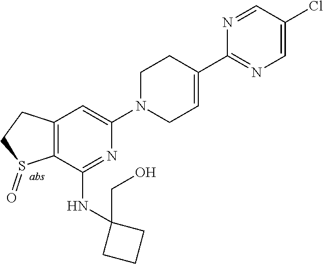 [SMILES: O=[S@]1CCc2cc(N3CC=C(c4ncc(Cl)cn4)CC3)nc(NC3(CO)CCC3)c21]

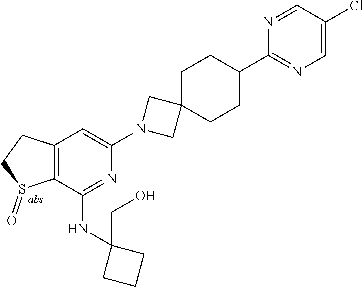 [SMILES: O=[S@]1CCc2cc(N3CC4(CCC(c5ncc(Cl)cn5)CC4)C3)nc(NC3(CO)CCC3)c21]

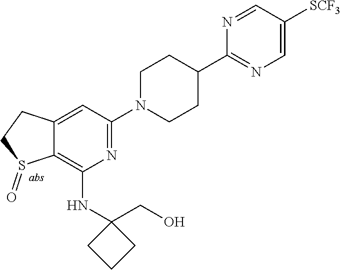

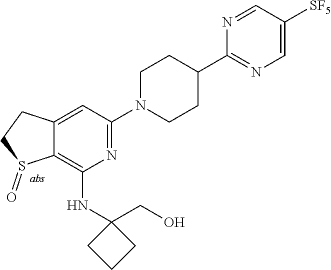

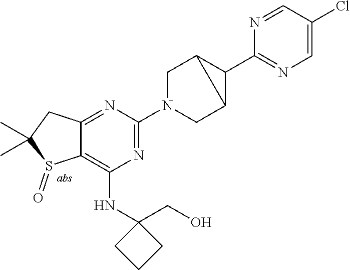 [SMILES: CC1(C)Cc2nc(N3CC4C(C3)C4c3ncc(Cl)cn3)nc(NC3(CO)CCC3)c2[S@]1=O]

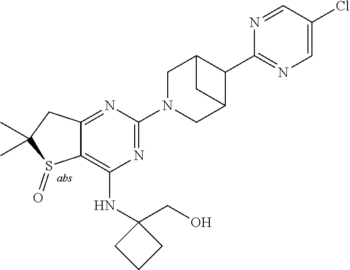 [SMILES: CC1(C)Cc2nc(N3CC4CC(C3)C4c3ncc(Cl)cn3)nc(NC3(CO)CCC3)c2[S@]1=O]

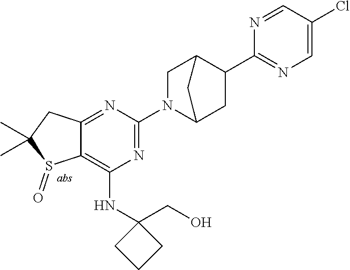 [SMILES: CC1(C)Cc2nc(N3CC4CC3CC4c3ncc(Cl)cn3)nc(NC3(CO)CCC3)c2[S@]1=O]

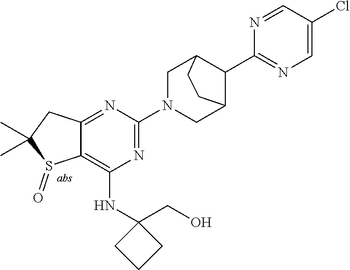 [SMILES: CC1(C)Cc2nc(N3CC4CCC(C3)C4c3ncc(Cl)cn3)nc(NC3(CO)CCC3)c2[S@]1=O]

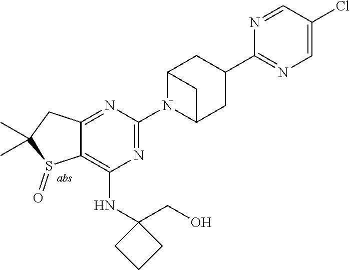 [SMILES: CC1(C)Cc2nc(N3C4CC(c5ncc(Cl)cn5)CC3C4)nc(NC3(CO)CCC3)c2[S@]1=O]

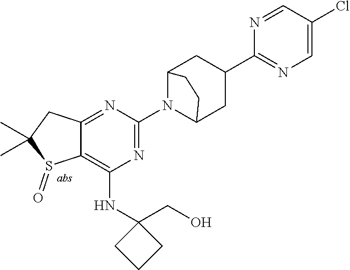 [SMILES: CC1(C)Cc2nc(N3C4CCC3CC(c3ncc(Cl)cn3)C4)nc(NC3(CO)CCC3)c2[S@]1=O]

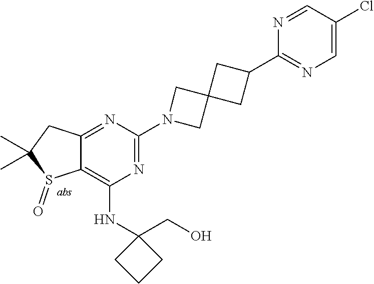 [SMILES: CC1(C)Cc2nc(N3CC4(CC(c5ncc(Cl)cn5)C4)C3)nc(NC3(CO)CCC3)c2[S@]1=O]

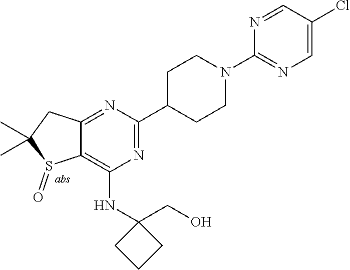 [SMILES: CC1(C)Cc2nc(C3CCN(c4ncc(Cl)cn4)CC3)nc(NC3(CO)CCC3)c2[S@]1=O]

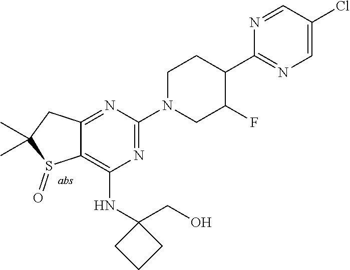 [SMILES: CC1(C)Cc2nc(N3CCC(c4ncc(Cl)cn4)C(F)C3)nc(NC3(CO)CCC3)c2[S@]1=O]

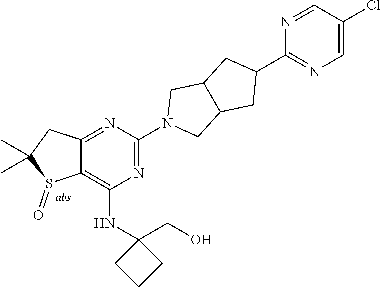 [SMILES: CC1(C)Cc2nc(N3CC4CC(c5ncc(Cl)cn5)CC4C3)nc(NC3(CO)CCC3)c2[S@]1=O]

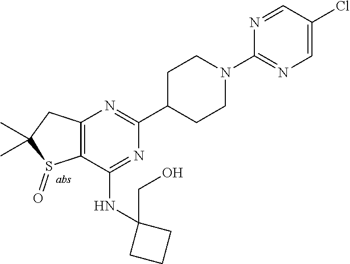 [SMILES: CC1(C)Cc2nc(C3CCN(c4ncc(Cl)cn4)CC3)nc(NC3(CO)CCC3)c2[S@]1=O]

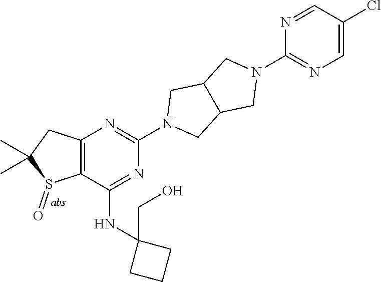 [SMILES: CC1(C)Cc2nc(N3CC4CN(c5ncc(Cl)cn5)CC4C3)nc(NC3(CO)CCC3)c2[S@]1=O]

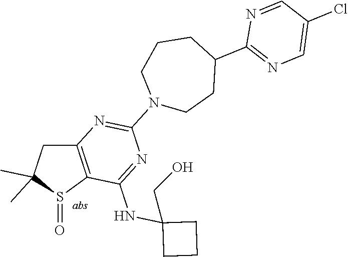 [SMILES: CC1(C)Cc2nc(N3CCCC(c4ncc(Cl)cn4)CC3)nc(NC3(CO)CCC3)c2[S@]1=O]

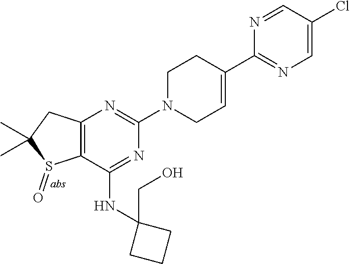 [SMILES: CC1(C)Cc2nc(N3CC=C(c4ncc(Cl)cn4)CC3)nc(NC3(CO)CCC3)c2[S@]1=O]

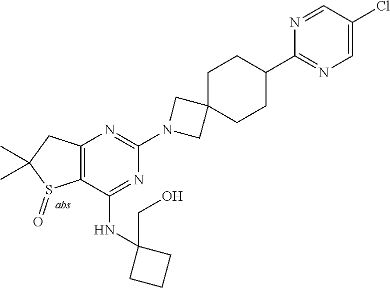 [SMILES: CC1(C)Cc2nc(N3CC4(CCC(c5ncc(Cl)cn5)CC4)C3)nc(NC3(CO)CCC3)c2S1=O]

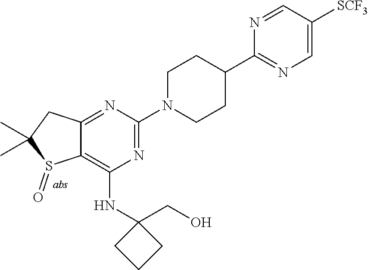

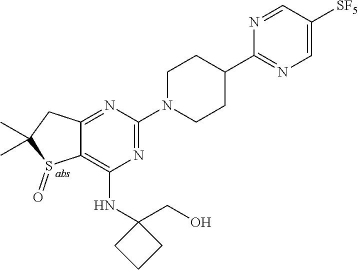

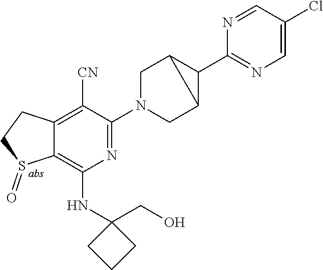 [SMILES: N#Cc1c(N2CC3C(C2)C3c2ncc(Cl)cn2)nc(NC2(CO)CCC2)c2c1CC[S@@]2=O]

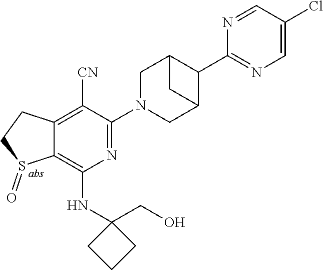 [SMILES: N#Cc1c(N2CC3CC(C2)C3c2ncc(Cl)cn2)nc(NC2(CO)CCC2)c2c1CC[S@@]2=O]

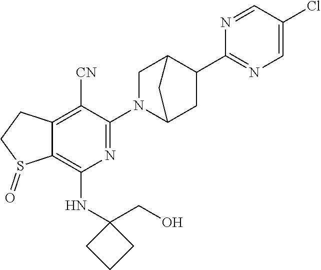 [SMILES: N#Cc1c(N2CC3CC2CC3c2ncc(Cl)cn2)nc(NC2(CO)CCC2)c2c1CCS2=O]

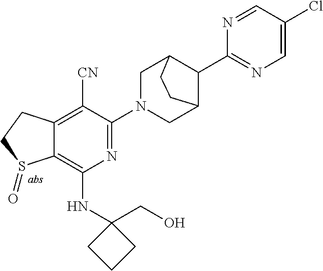 [SMILES: N#Cc1c(N2CC3CCC(C2)C3c2ncc(Cl)cn2)nc(NC2(CO)CCC2)c2c1CC[S@@]2=O]

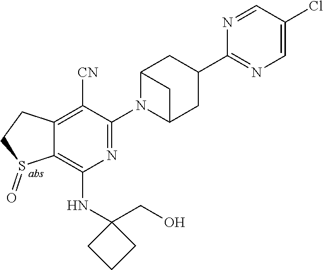 [SMILES: N#Cc1c(N2C3CC(c4ncc(Cl)cn4)CC2C3)nc(NC2(CO)CCC2)c2c1CC[S@@]2=O]

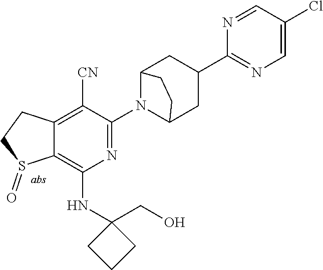 [SMILES: N#Cc1c(N2C3CCC2CC(c2ncc(Cl)cn2)C3)nc(NC2(CO)CCC2)c2c1CC[S@@]2=O]

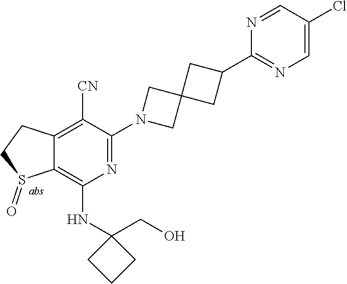 [SMILES: N#Cc1c(N2CC3(CC(c4ncc(Cl)cn4)C3)C2)nc(NC2(CO)CCC2)c2c1CC[S@@]2=O]

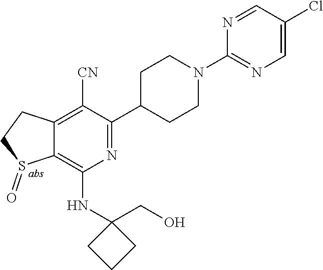 [SMILES: N#Cc1c(C2CCN(c3ncc(Cl)cn3)CC2)nc(NC2(CO)CCC2)c2c1CC[S@@]2=O]

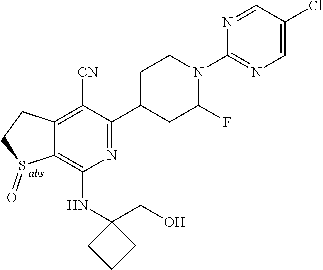 [SMILES: N#Cc1c(C2CCN(c3ncc(Cl)cn3)C(F)C2)nc(NC2(CO)CCC2)c2c1CC[S@@]2=O]

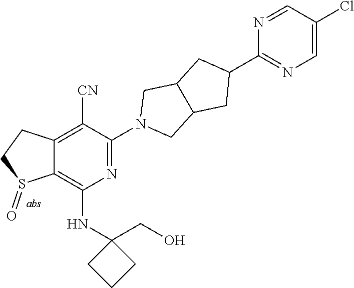 [SMILES: N#Cc1c(N2CC3CC(c4ncc(Cl)cn4)CC3C2)nc(NC2(CO)CCC2)c2c1CC[S@@]2=O]

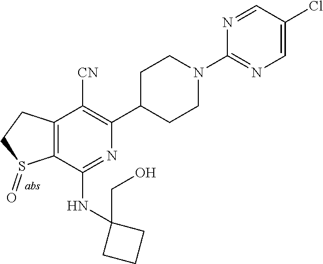 [SMILES: N#Cc1c(C2CCN(c3ncc(Cl)cn3)CC2)nc(NC2(CO)CCC2)c2c1CC[S@@]2=O]

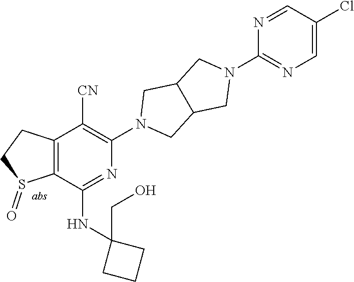 [SMILES: N#Cc1c(N2CC3CN(c4ncc(Cl)cn4)CC3C2)nc(NC2(CO)CCC2)c2c1CC[S@@]2=O]

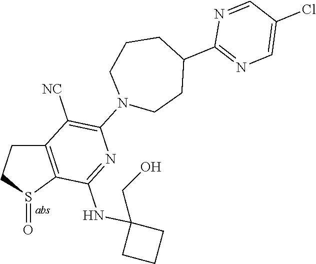 [SMILES: N#Cc1c(N2CCCC(c3ncc(Cl)cn3)CC2)nc(NC2(CO)CCC2)c2c1CC[S@@]2=O]

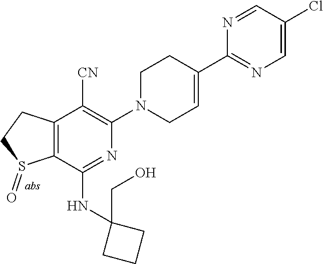 [SMILES: N#Cc1c(N2CC=C(c3ncc(Cl)cn3)CC2)nc(NC2(CO)CCC2)c2c1CC[S@@]2=O]

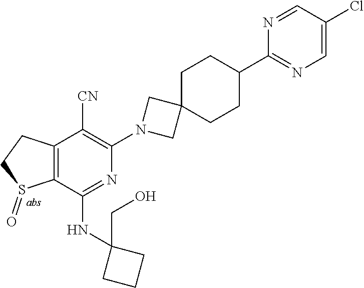 [SMILES: N#Cc1c(N2CC3(CCC(c4ncc(Cl)cn4)CC3)C2)nc(NC2(CO)CCC2)c2c1CC[S@@]2=O]

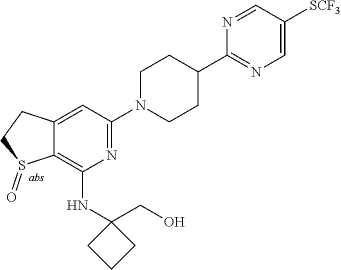

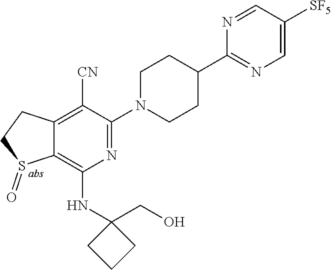

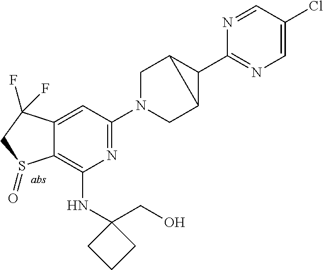 [SMILES: O=[S@]1CC(F)(F)c2cc(N3CC4C(C3)C4c3ncc(Cl)cn3)nc(NC3(CO)CCC3)c21]

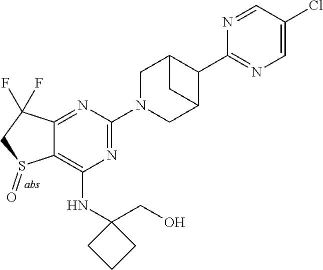 [SMILES: O=[S@]1CC(F)(F)c2nc(N3CC4CC(C3)C4c3ncc(Cl)cn3)nc(NC3(CO)CCC3)c21]

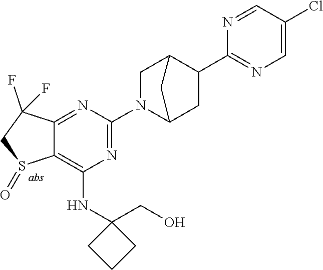 [SMILES: O=[S@]1CC(F)(F)c2nc(N3CC4CC3CC4c3ncc(Cl)cn3)nc(NC3(CO)CCC3)c21]

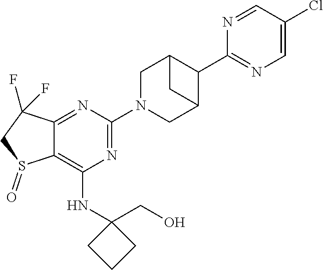 [SMILES: O=[S@]1CC(F)(F)c2nc(N3CC4CC(C3)C4c3ncc(Cl)cn3)nc(NC3(CO)CCC3)c21]

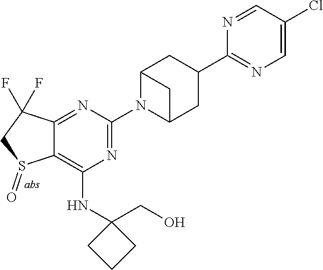 [SMILES: O=[S@]1CC(F)(F)c2nc(N3C4CC(c5ncc(Cl)cn5)CC3C4)nc(NC3(CO)CCC3)c21]

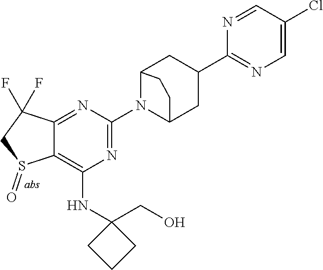 [SMILES: O=[S@]1CC(F)(F)c2nc(N3C4CCC3CC(c3ncc(Cl)cn3)C4)nc(NC3(CO)CCC3)c21]

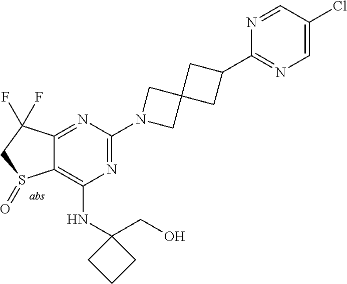 [SMILES: O=[S@]1CC(F)(F)c2nc(N3CC4(CC(c5ncc(Cl)cn5)C4)C3)nc(NC3(CO)CCC3)c21]

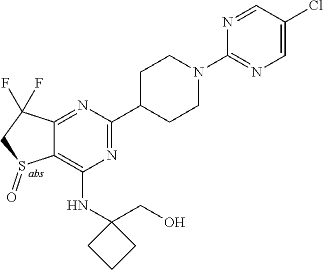 [SMILES: O=[S@]1CC(F)(F)c2nc(C3CCN(c4ncc(Cl)cn4)CC3)nc(NC3(CO)CCC3)c21]

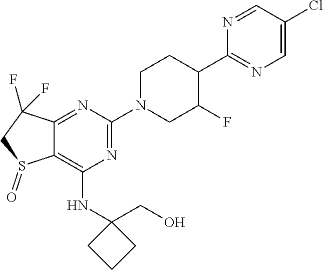 [SMILES: O=[S@]1CC(F)(F)c2nc(N3CCC(c4ncc(Cl)cn4)C(F)C3)nc(NC3(CO)CCC3)c21]

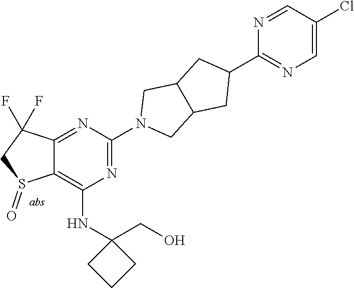 [SMILES: O=[S@]1CC(F)(F)c2nc(N3CC4CC(c5ncc(Cl)cn5)CC4C3)nc(NC3(CO)CCC3)c21]

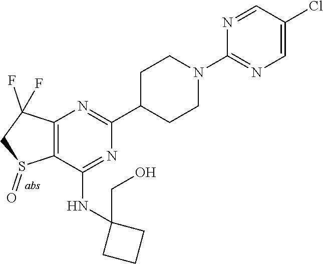 [SMILES: O=[S@]1CC(F)(F)c2nc(C3CCN(c4ncc(Cl)cn4)CC3)nc(NC3(CO)CCC3)c21]

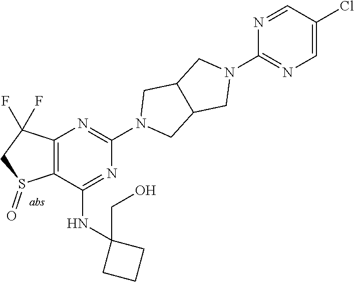 [SMILES: O=[S@]1CC(F)(F)c2nc(N3CC4CN(c5ncc(Cl)cn5)CC4C3)nc(NC3(CO)CCC3)c21]

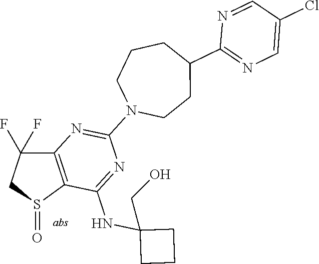 [SMILES: O=[S@]1CC(F)(F)c2nc(N3CCCC(c4ncc(Cl)cn4)CC3)nc(NC3(CO)CCC3)c21]

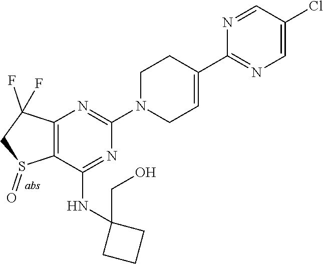 [SMILES: O=[S@]1CC(F)(F)c2nc(N3CC=C(c4ncc(Cl)cn4)CC3)nc(NC3(CO)CCC3)c21]

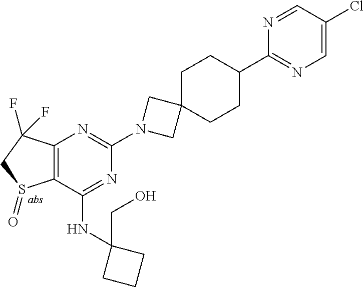 [SMILES: O=[S@]1CC(F)(F)c2nc(N3CC4(CCC(c5ncc(Cl)cn5)CC4)C3)nc(NC3(CO)CCC3)c21]

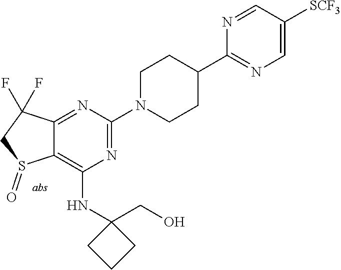

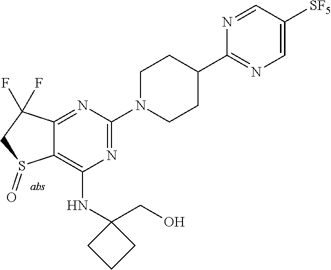

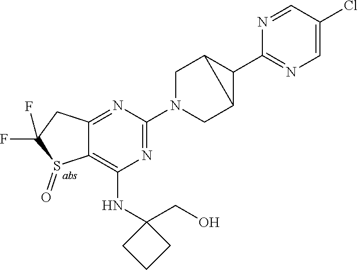 [SMILES: O=[S@@]1c2c(nc(N3CC4C(C3)C4c3ncc(Cl)cn3)nc2NC2(CO)CCC2)CC1(F)F]

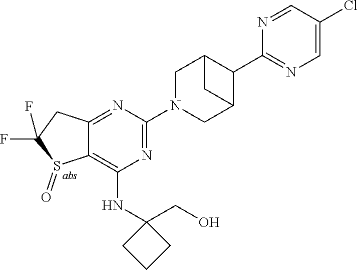 [SMILES: O=[S@@]1c2c(nc(N3CC4CC(C3)C4c3ncc(Cl)cn3)nc2NC2(CO)CCC2)CC1(F)F]

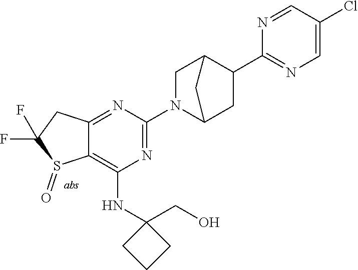 [SMILES: O=[S@@]1c2c(nc(N3CC4CC3CC4c3ncc(Cl)cn3)nc2NC2(CO)CCC2)CC1(F)F]

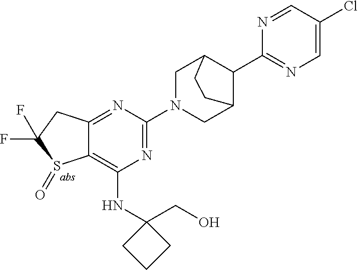 [SMILES: O=[S@@]1c2c(nc(N3CC4CCC(C3)C4c3ncc(Cl)cn3)nc2NC2(CO)CCC2)CC1(F)F]

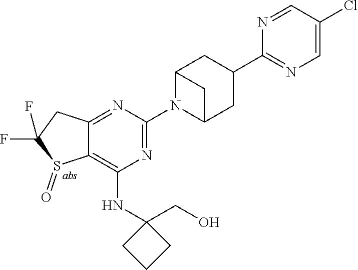 [SMILES: O=[S@@]1c2c(nc(N3C4CC(c5ncc(Cl)cn5)CC3C4)nc2NC2(CO)CCC2)CC1(F)F]

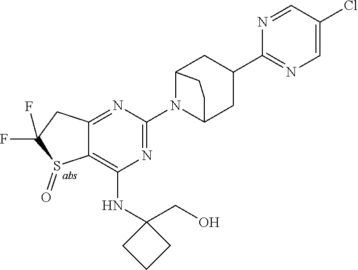 [SMILES: O=[S@@]1c2c(nc(N3C4CCC3CC(c3ncc(Cl)cn3)C4)nc2NC2(CO)CCC2)CC1(F)F]

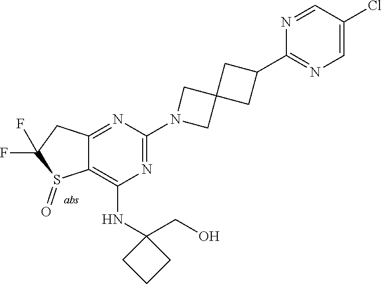 [SMILES: O=[S@@]1c2c(nc(N3CC4(CC(c5ncc(Cl)cn5)C4)C3)nc2NC2(CO)CCC2)CC1(F)F]

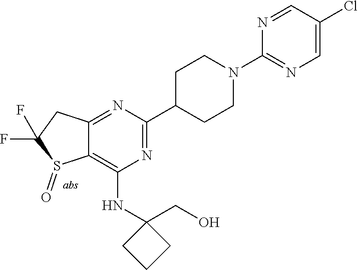 [SMILES: O=[S@@]1c2c(nc(C3CCN(c4ncc(Cl)cn4)CC3)nc2NC2(CO)CCC2)CC1(F)F]

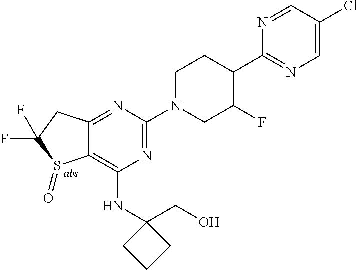 [SMILES: O=[S@@]1c2c(nc(N3CCC(c4ncc(Cl)cn4)C(F)C3)nc2NC2(CO)CCC2)CC1(F)F]

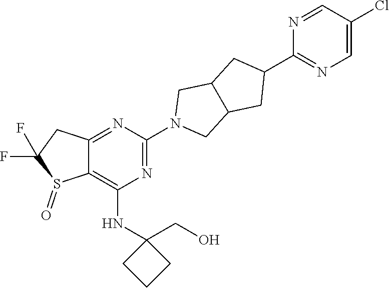 [SMILES: O=[S@@]1c2c(nc(N3CC4CC(c5ncc(Cl)cn5)CC4C3)nc2NC2(CO)CCC2)CC1(F)F]

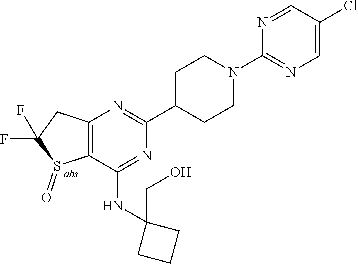 [SMILES: O=[S@@]1c2c(nc(C3CCN(c4ncc(Cl)cn4)CC3)nc2NC2(CO)CCC2)CC1(F)F]

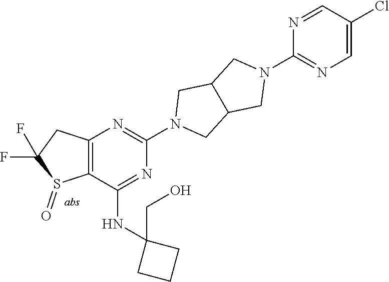 [SMILES: O=[S@@]1c2c(nc(N3CC4CN(c5ncc(Cl)cn5)CC4C3)nc2NC2(CO)CCC2)CC1(F)F]

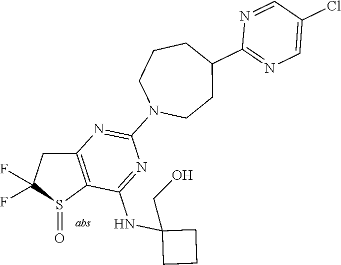 [SMILES: O=[S@@]1c2c(nc(N3CCCC(c4ncc(Cl)cn4)CC3)nc2NC2(CO)CCC2)CC1(F)F]

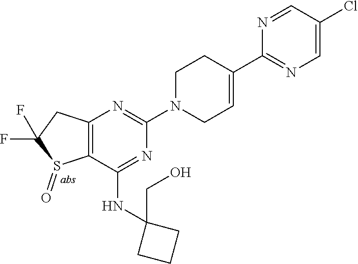 [SMILES: O=[S@@]1c2c(nc(N3CC=C(c4ncc(Cl)cn4)CC3)nc2NC2(CO)CCC2)CC1(F)F]

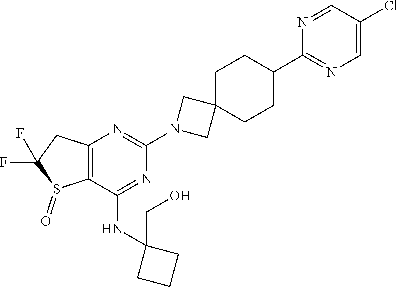 [SMILES: O=[S@@]1c2c(nc(N3CC4(CCC(c5ncc(Cl)cn5)CC4)C3)nc2NC2(CO)CCC2)CC1(F)F]

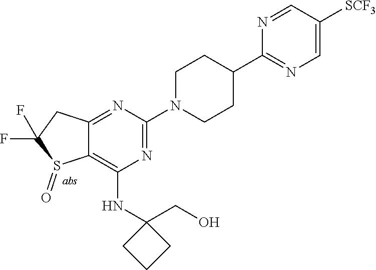

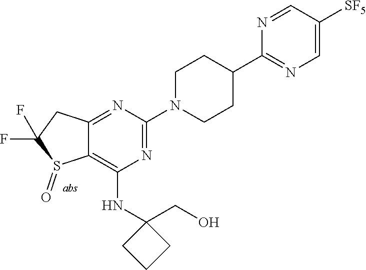

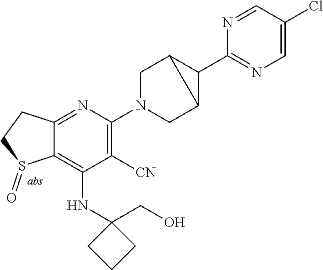 [SMILES: N#Cc1c(N2CC3C(C2)C3c2ncc(Cl)cn2)nc2c(c1NC1(CO)CCC1)[S@@](=O)CC2]

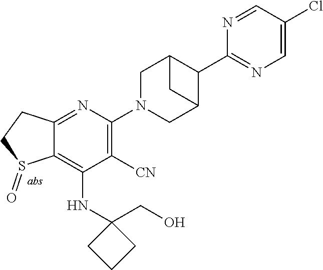 [SMILES: N#Cc1c(N2CC3CC(C2)C3c2ncc(Cl)cn2)nc2c(c1NC1(CO)CCC1)[S@@](=O)CC2]

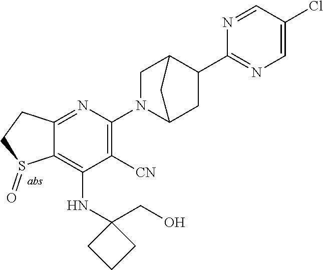 [SMILES: N#Cc1c(N2CC3CC2CC3c2ncc(Cl)cn2)nc2c(c1NC1(CO)CCC1)[S@@](=O)CC2]

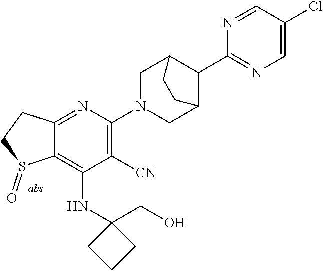 [SMILES: N#Cc1c(N2CC3CCC(C2)C3c2ncc(Cl)cn2)nc2c(c1NC1(CO)CCC1)[S@@](=O)CC2]

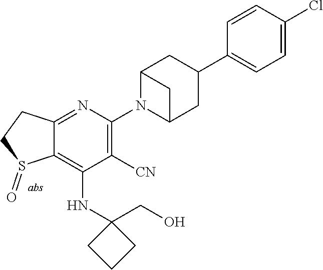 [SMILES: N#Cc1c(N2C3CC(c4ccc(Cl)cc4)CC2C3)nc2c(c1NC1(CO)CCC1)[S@@](=O)CC2]

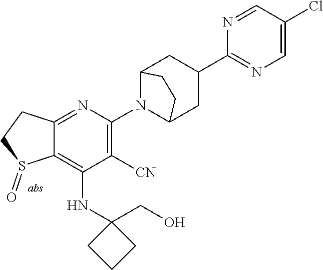 [SMILES: N#Cc1c(N2C3CCC2CC(c2ncc(Cl)cn2)C3)nc2c(c1NC1(CO)CCC1)[S@@](=O)CC2]

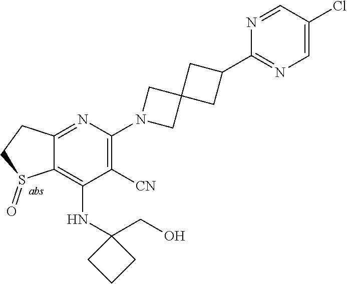 [SMILES: N#Cc1c(N2CC3(CC(c4ncc(Cl)cn4)C3)C2)nc2c(c1NC1(CO)CCC1)[S@@](=O)CC2]

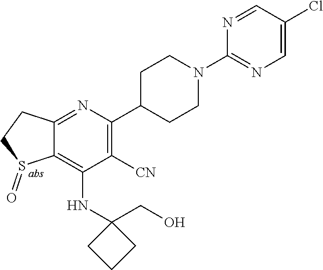 [SMILES: N#Cc1c(C2CCN(c3ncc(Cl)cn3)CC2)nc2c(c1NC1(CO)CCC1)[S@@](=O)CC2]

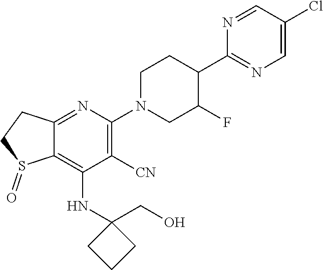 [SMILES: N#Cc1c(N2CCC(c3ncc(Cl)cn3)C(F)C2)nc2c(c1NC1(CO)CCC1)[S@@](=O)CC2]

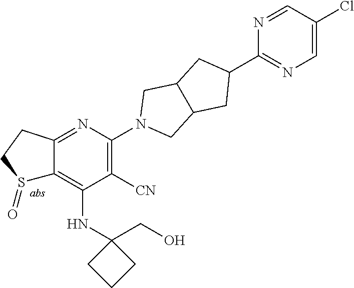 [SMILES: N#Cc1c(N2CC3CC(c4ncc(Cl)cn4)CC3C2)nc2c(c1NC1(CO)CCC1)[S@@](=O)CC2]

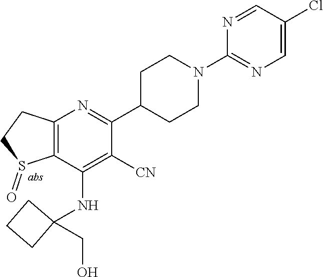 [SMILES: N#Cc1c(C2CCN(c3ncc(Cl)cn3)CC2)nc2c(c1NC1(CO)CCC1)[S@@](=O)CC2]

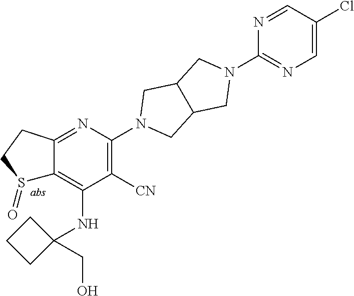 [SMILES: N#Cc1c(N2CC3CN(c4ncc(Cl)cn4)CC3C2)nc2c(c1NC1(CO)CCC1)[S@@](=O)CC2]

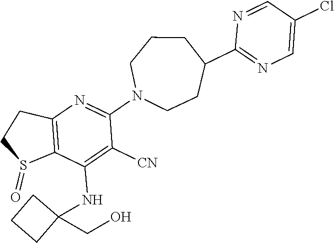 [SMILES: N#Cc1c(N2CCCC(c3ncc(Cl)cn3)CC2)nc2c(c1NC1(CO)CCC1)[S@@](=O)CC2]

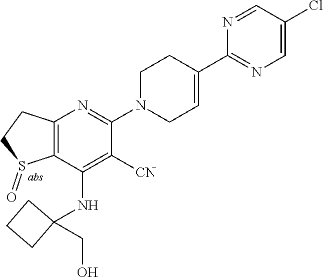 [SMILES: N#Cc1c(N2CC=C(c3ncc(Cl)cn3)CC2)nc2c(c1NC1(CO)CCC1)[S@@](=O)CC2]

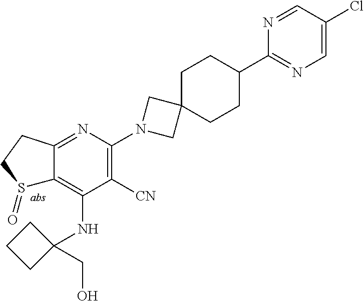 [SMILES: N#Cc1c(N2CC3(CCC(c4ncc(Cl)cn4)CC3)C2)nc2c(c1NC1(CO)CCC1)[S@@](=O)CC2]

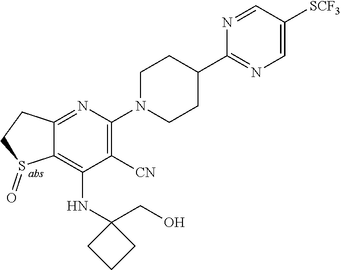

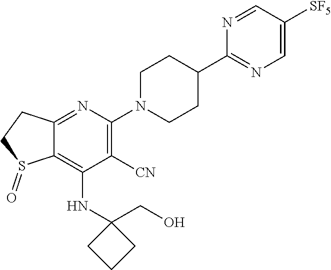

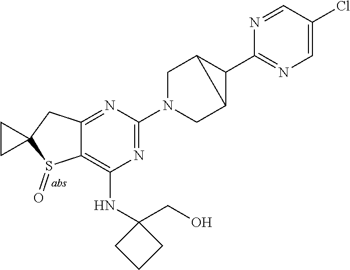 [SMILES: O=[S@@]1c2c(nc(N3CC4C(C3)C4c3ncc(Cl)cn3)nc2NC2(CO)CCC2)CC12CC2]

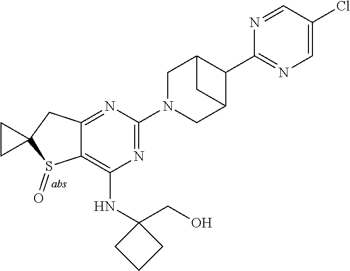 [SMILES: O=[S@@]1c2c(nc(N3CC4CC(C3)C4c3ncc(Cl)cn3)nc2NC2(CO)CCC2)CC12CC2]

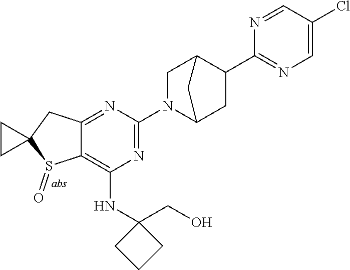 [SMILES: O=[S@@]1c2c(nc(N3CC4CC3CC4c3ncc(Cl)cn3)nc2NC2(CO)CCC2)CC12CC2]

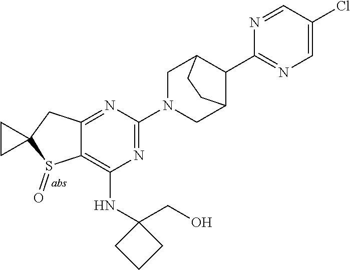 [SMILES: O=[S@@]1c2c(nc(N3CC4CCC(C3)C4c3ncc(Cl)cn3)nc2NC2(CO)CCC2)CC12CC2]

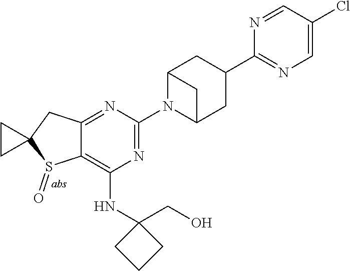 [SMILES: O=[S@@]1c2c(nc(N3C4CC(c5ncc(Cl)cn5)CC3C4)nc2NC2(CO)CCC2)CC12CC2]

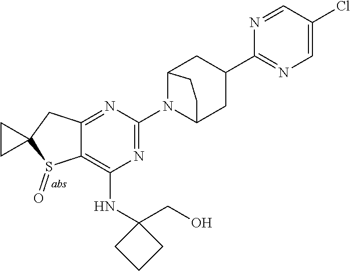 [SMILES: O=[S@@]1c2c(nc(N3C4CCC3CC(c3ncc(Cl)cn3)C4)nc2NC2(CO)CCC2)CC12CC2]

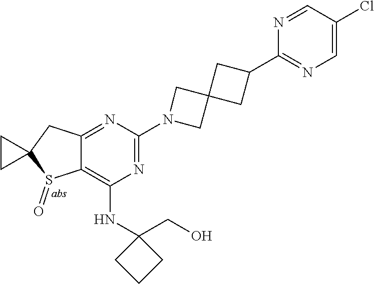 [SMILES: O=[S@@]1c2c(nc(N3CC4(CC(c5ncc(Cl)cn5)C4)C3)nc2NC2(CO)CCC2)CC12CC2]

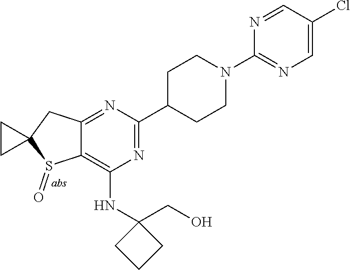 [SMILES: O=[S@@]1c2c(nc(C3CCN(c4ncc(Cl)cn4)CC3)nc2NC2(CO)CCC2)CC12CC2]

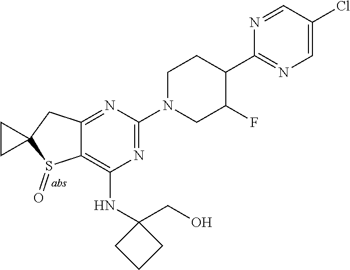 [SMILES: O=[S@@]1c2c(nc(N3CCC(c4ncc(Cl)cn4)C(F)C3)nc2NC2(CO)CCC2)CC12CC2]

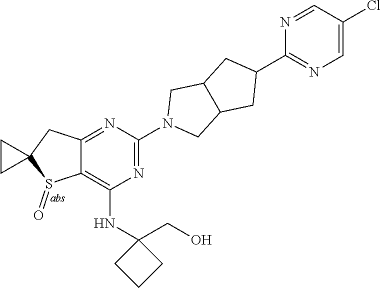 [SMILES: O=[S@@]1c2c(nc(N3CC4CC(c5ncc(Cl)cn5)CC4C3)nc2NC2(CO)CCC2)CC12CC2]

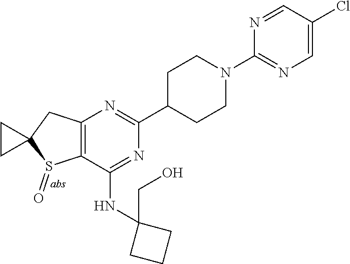 [SMILES: O=[S@@]1c2c(nc(C3CCN(c4ncc(Cl)cn4)CC3)nc2NC2(CO)CCC2)CC12CC2]

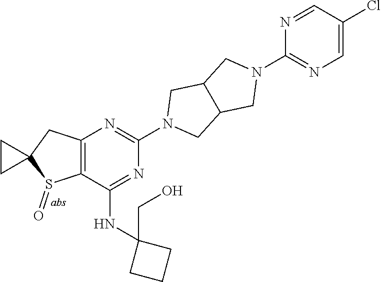 [SMILES: O=[S@@]1c2c(nc(N3CC4CN(c5ncc(Cl)cn5)CC4C3)nc2NC2(CO)CCC2)CC12CC2]

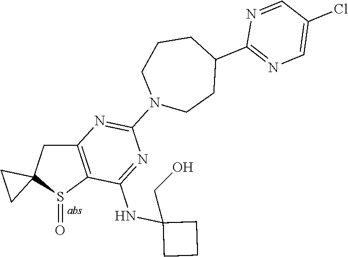 [SMILES: O=[S@@]1c2c(nc(N3CCCC(c4ncc(Cl)cn4)CC3)nc2NC2(CO)CCC2)CC12CC2]

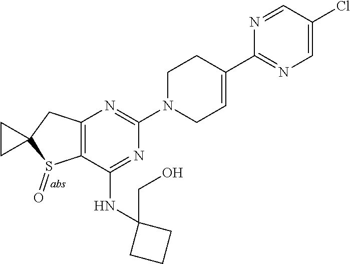 [SMILES: O=[S@@]1c2c(nc(N3CC=C(c4ncc(Cl)cn4)CC3)nc2NC2(CO)CCC2)CC12CC2]

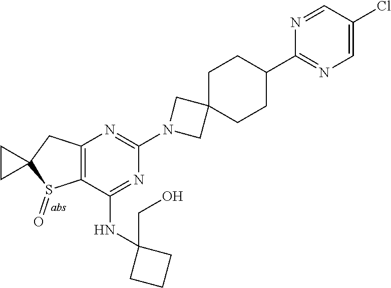 [SMILES: O=[S@@]1c2c(nc(N3CC4(CCC(c5ncc(Cl)cn5)CC4)C3)nc2NC2(CO)CCC2)CC12CC2]

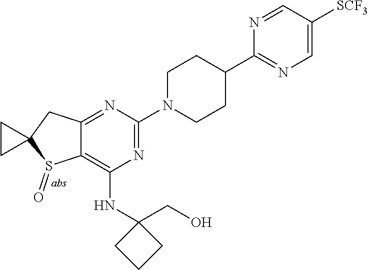

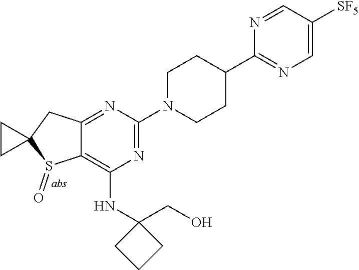

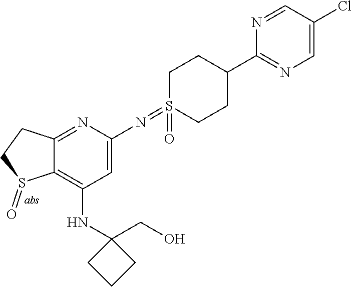 [SMILES: O=[S@]1CCc2nc(N=S3(=O)CCC(c4ncc(Cl)cn4)CC3)cc(NC3(CO)CCC3)c21]

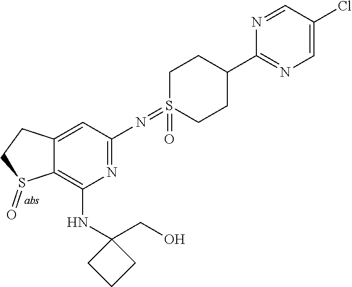 [SMILES: O=[S@]1CCc2cc(N=S3(=O)CCC(c4ncc(Cl)cn4)CC3)nc(NC3(CO)CCC3)c21]

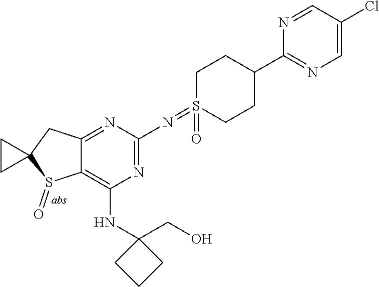 [SMILES: O=[S@@]1c2c(nc(N=S3(=O)CCC(c4ncc(Cl)cn4)CC3)nc2NC2(CO)CCC2)CC12CC2]

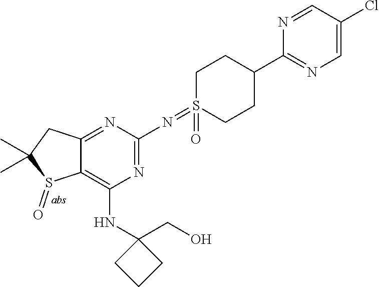 [SMILES: CC1(C)Cc2nc(N=S3(=O)CCC(c4ncc(Cl)cn4)CC3)nc(NC3(CO)CCC3)c2[S@]1=O]

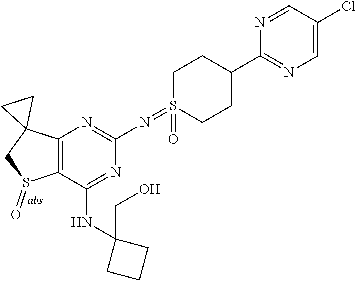 [SMILES: O=[S@]1CC2(CC2)c2nc(N=S3(=O)CCC(c4ncc(Cl)cn4)CC3)nc(NC3(CO)CCC3)c21]

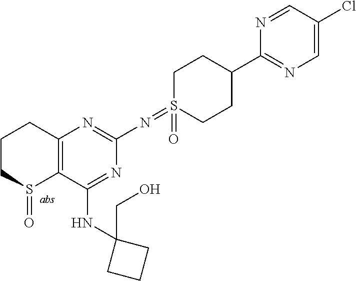 [SMILES: O=[S@]1CCCc2nc(N=S3(=O)CCC(c4ncc(Cl)cn4)CC3)nc(NC3(CO)CCC3)c21]

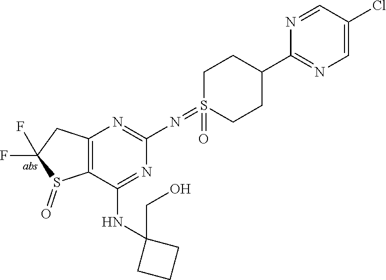

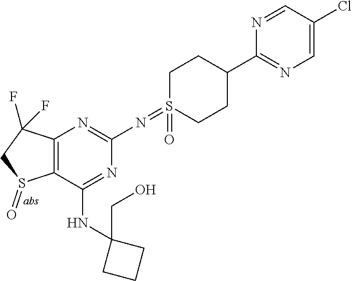 [SMILES: O=[S@]1CC(F)(F)c2nc(N=S3(=O)CCC(c4ncc(Cl)cn4)CC3)nc(NC3(CO)CCC3)c21]

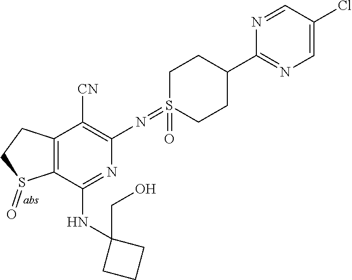 [SMILES: N#Cc1c(N=S2(=O)CCC(c3ncc(Cl)cn3)CC2)nc(NC2(CO)CCC2)c2c1CC[S@@]2=O]

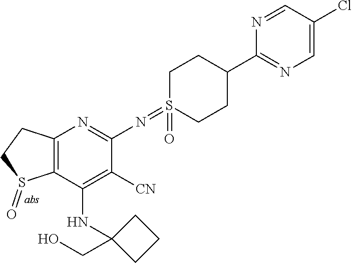 [SMILES: N#Cc1c(N=S2(=O)CCC(c3ncc(Cl)cn3)CC2)nc2c(c1NC1(CO)CCC1)[S@@](=O)CC2]

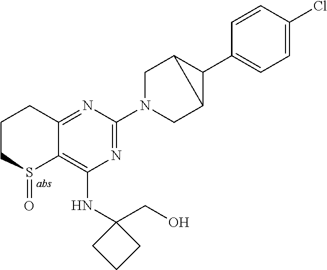 [SMILES: O=[S@]1CCCc2nc(N3CC4C(C3)C4c3ccc(Cl)cc3)nc(NC3(CO)CCC3)c21]

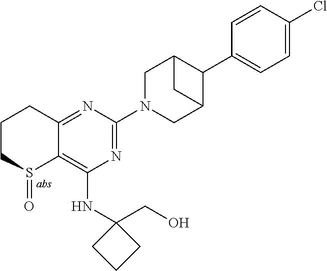 [SMILES: O=[S@]1CCCc2nc(N3CC4CC(C3)C4c3ccc(Cl)cc3)nc(NC3(CO)CCC3)c21]

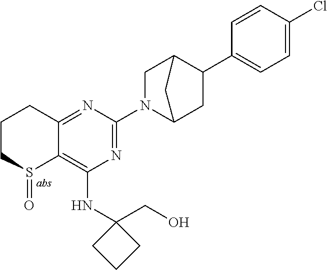 [SMILES: O=[S@]1CCCc2nc(N3CC4CC3CC4c3ccc(Cl)cc3)nc(NC3(CO)CCC3)c21]

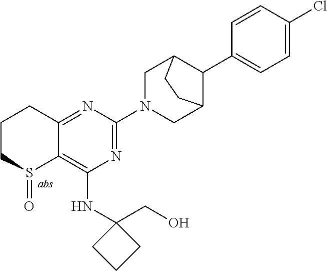 [SMILES: O=[S@]1CCCc2nc(N3CC4CCC(C3)C4c3ccc(Cl)cc3)nc(NC3(CO)CCC3)c21]

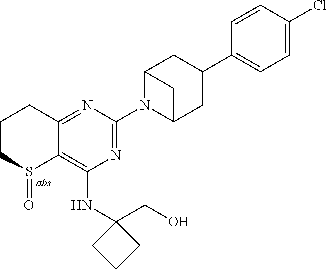 [SMILES: O=[S@]1CCCc2nc(N3C4CC(c5ccc(Cl)cc5)CC3C4)nc(NC3(CO)CCC3)c21]

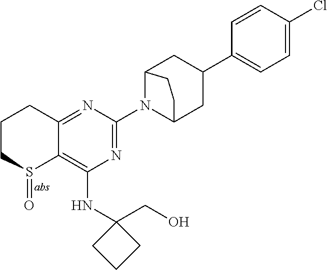 [SMILES: O=[S@]1CCCc2nc(N3C4CCC3CC(c3ccc(Cl)cc3)C4)nc(NC3(CO)CCC3)c21]

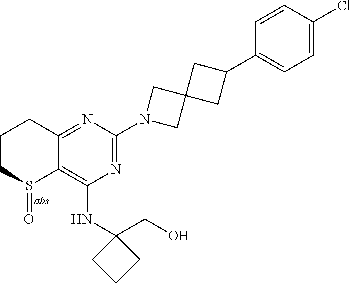 [SMILES: O=[S@]1CCCc2nc(N3CC4(CC(c5ccc(Cl)cc5)C4)C3)nc(NC3(CO)CCC3)c21]

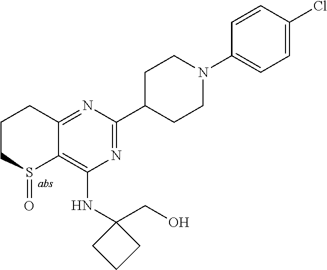 [SMILES: O=[S@]1CCCc2nc(C3CCN(c4ccc(Cl)cc4)CC3)nc(NC3(CO)CCC3)c21]

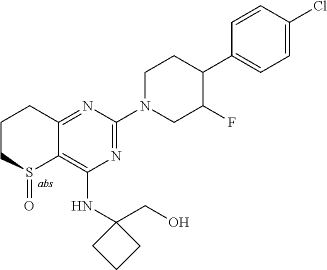 [SMILES: O=[S@]1CCCc2nc(N3CCC(c4ccc(Cl)cc4)C(F)C3)nc(NC3(CO)CCC3)c21]

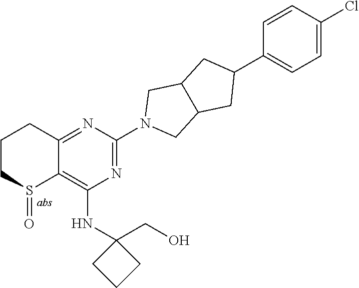 [SMILES: O=[S@]1CCCc2nc(N3CC4CC(c5ccc(Cl)cc5)CC4C3)nc(NC3(CO)CCC3)c21]

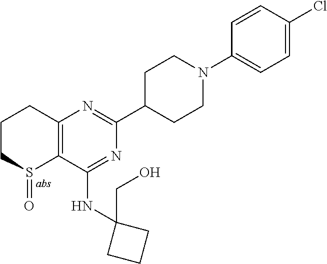 [SMILES: O=[S@]1CCCc2nc(C3CCN(c4ccc(Cl)cc4)CC3)nc(NC3(CO)CCC3)c21]

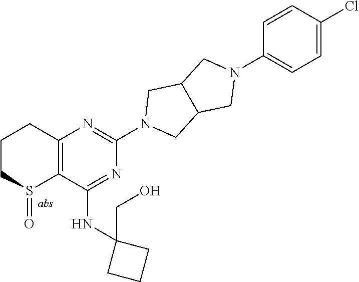 [SMILES: O=[S@]1CCCc2nc(N3CC4CN(c5ccc(Cl)cc5)CC4C3)nc(NC3(CO)CCC3)c21]

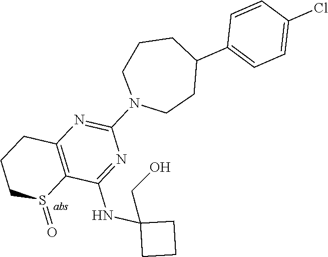 [SMILES: O=[S@]1CCCc2nc(N3CCCC(c4ccc(Cl)cc4)CC3)nc(NC3(CO)CCC3)c21]

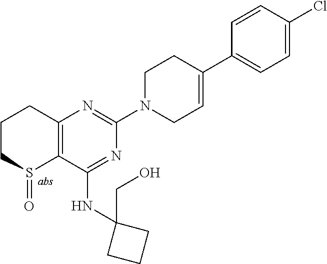 [SMILES: O=[S@]1CCCc2nc(N3CC=C(c4ccc(Cl)cc4)CC3)nc(NC3(CO)CCC3)c21]

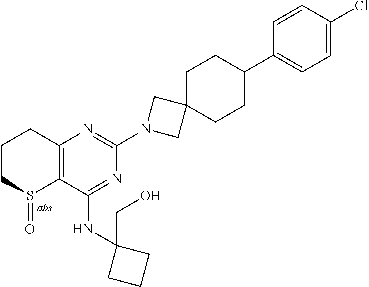 [SMILES: O=[S@]1CCCc2nc(N3CC4(CCC(c5ccc(Cl)cc5)CC4)C3)nc(NC3(CO)CCC3)c21]

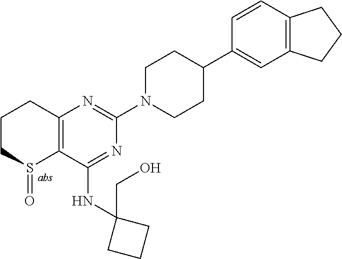 [SMILES: O=[S@]1CCCc2nc(N3CCC(c4ccc5c(c4)CCC5)CC3)nc(NC3(CO)CCC3)c21]

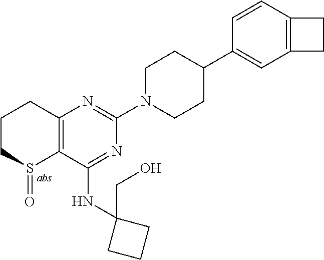 [SMILES: O=[S@]1CCCc2nc(N3CCC(c4ccc5c(c4)CC5)CC3)nc(NC3(CO)CCC3)c21]

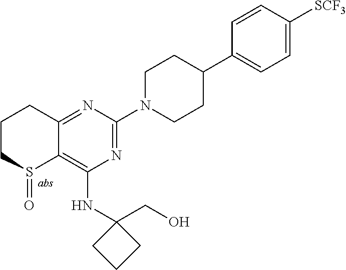

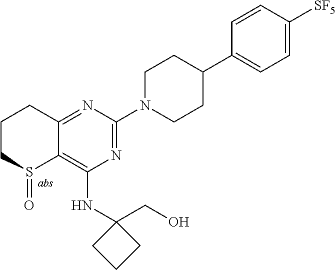

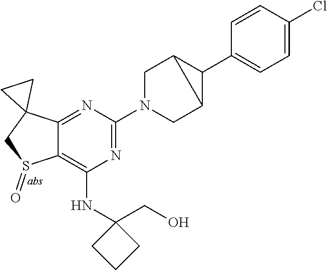 [SMILES: O=[S@]1CC2(CC2)c2nc(N3CC4C(C3)C4c3ccc(Cl)cc3)nc(NC3(CO)CCC3)c21]

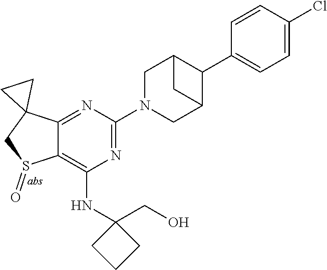 [SMILES: O=[S@]1CC2(CC2)c2nc(N3CC4CC(C3)C4c3ccc(Cl)cc3)nc(NC3(CO)CCC3)c21]

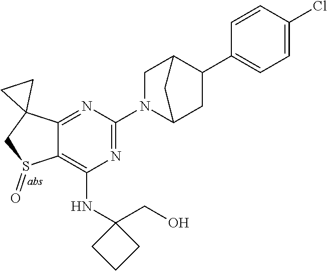 [SMILES: O=[S@]1CC2(CC2)c2nc(N3CC4CC3CC4c3ccc(Cl)cc3)nc(NC3(CO)CCC3)c21]

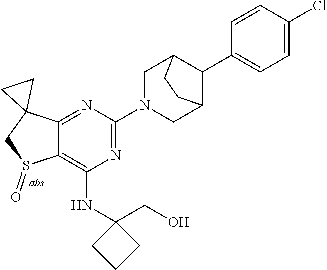 [SMILES: O=[S@]1CC2(CC2)c2nc(N3CC4CCC(C3)C4c3ccc(Cl)cc3)nc(NC3(CO)CCC3)c21]

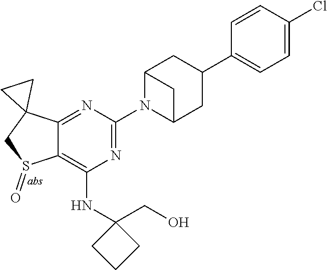 [SMILES: O=[S@]1CC2(CC2)c2nc(N3C4CC(c5ccc(Cl)cc5)CC3C4)nc(NC3(CO)CCC3)c21]

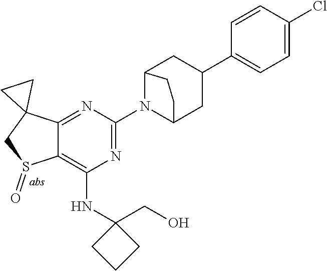 [SMILES: O=[S@]1CC2(CC2)c2nc(N3C4CCC3CC(c3ccc(Cl)cc3)C4)nc(NC3(CO)CCC3)c21]

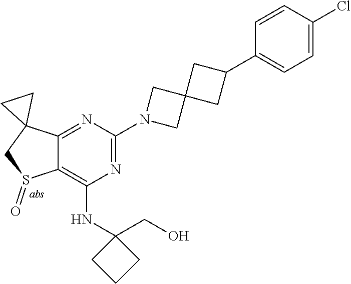 [SMILES: O=[S@]1CC2(CC2)c2nc(N3CC4(CC(c5ccc(Cl)cc5)C4)C3)nc(NC3(CO)CCC3)c21]

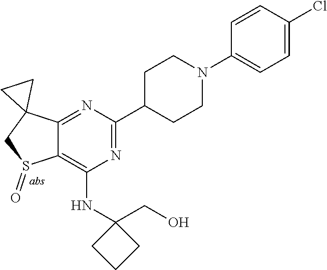 [SMILES: O=[S@]1CC2(CC2)c2nc(C3CCN(c4ccc(Cl)cc4)CC3)nc(NC3(CO)CCC3)c21]

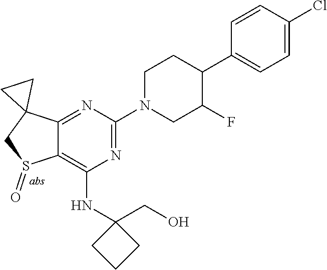 [SMILES: O=[S@]1CC2(CC2)c2nc(N3CCC(c4ccc(Cl)cc4)C(F)C3)nc(NC3(CO)CCC3)c21]

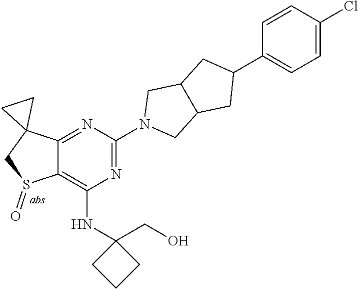 [SMILES: O=[S@]1CC2(CC2)c2nc(N3CC4CC(c5ccc(Cl)cc5)CC4C3)nc(NC3(CO)CCC3)c21]

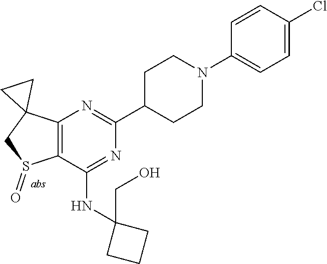 [SMILES: O=[S@]1CC2(CC2)c2nc(C3CCN(c4ccc(Cl)cc4)CC3)nc(NC3(CO)CCC3)c21]

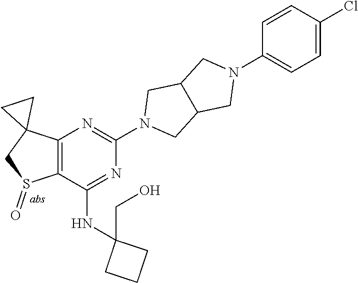 [SMILES: O=[S@]1CC2(CC2)c2nc(N3CC4CN(c5ccc(Cl)cc5)CC4C3)nc(NC3(CO)CCC3)c21]

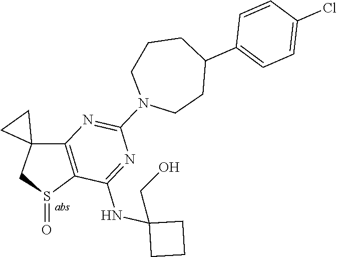 [SMILES: O=[S@]1CC2(CC2)c2nc(N3CCCC(c4ccc(Cl)cc4)CC3)nc(NC3(CO)CCC3)c21]

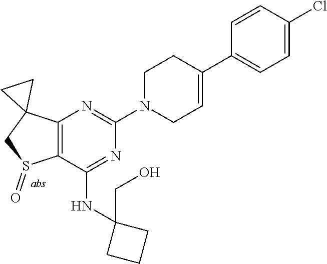 [SMILES: O=[S@]1CC2(CC2)c2nc(N3CC=C(c4ccc(Cl)cc4)CC3)nc(NC3(CO)CCC3)c21]

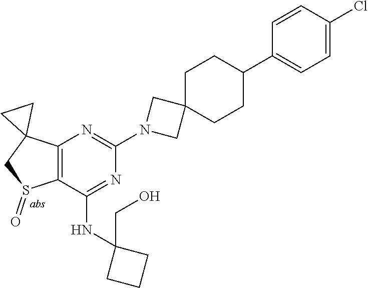 [SMILES: O=[S@]1CC2(CC2)c2nc(N3CC4(CCC(c5ccc(Cl)cc5)CC4)C3)nc(NC3(CO)CCC3)c21]

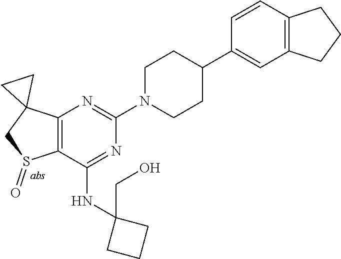 [SMILES: O=[S@]1CC2(CC2)c2nc(N3CCC(c4ccc5c(c4)CCC5)CC3)nc(NC3(CO)CCC3)c21]

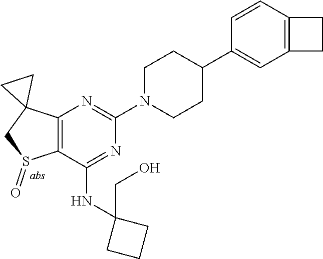 [SMILES: O=[S@]1CC2(CC2)c2nc(N3CCC(c4ccc5c(c4)CC5)CC3)nc(NC3(CO)CCC3)c21]

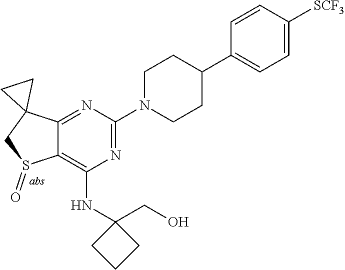

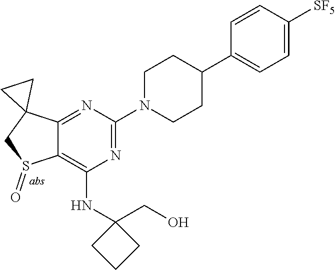

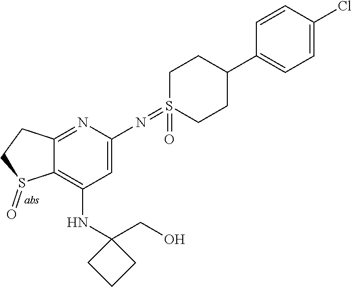 [SMILES: O=[S@]1CCc2nc(N=S3(=O)CCC(c4ccc(Cl)cc4)CC3)cc(NC3(CO)CCC3)c21]

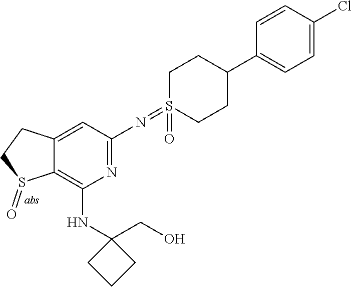 [SMILES: O=[S@]1CCc2cc(N=S3(=O)CCC(c4ccc(Cl)cc4)CC3)nc(NC3(CO)CCC3)c21]

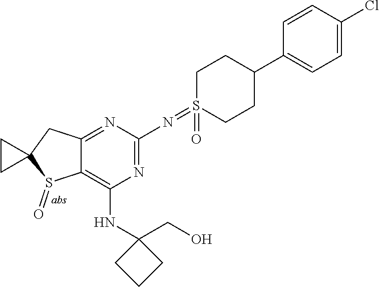 [SMILES: O=[S@@]1c2c(nc(N=S3(=O)CCC(c4ccc(Cl)cc4)CC3)nc2NC2(CO)CCC2)CC12CC2]

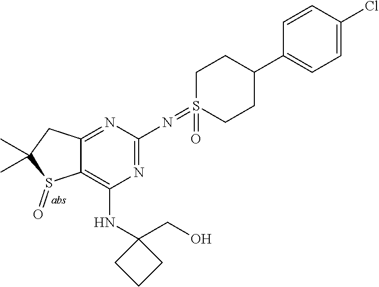 [SMILES: CC1(C)Cc2nc(N=S3(=O)CCC(c4ccc(Cl)cc4)CC3)nc(NC3(CO)CCC3)c2[S@]1=O]

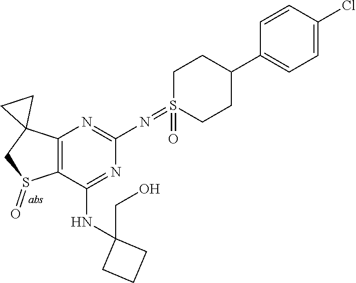 [SMILES: O=[S@]1CC2(CC2)c2nc(N=S3(=O)CCC(c4ccc(Cl)cc4)CC3)nc(NC3(CO)CCC3)c21]

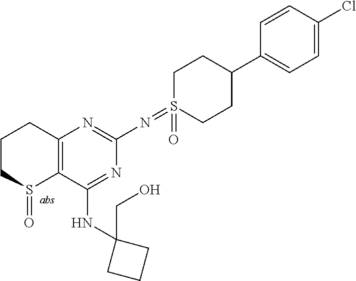 [SMILES: O=[S@]1CCCc2nc(N=S3(=O)CCC(c4ccc(Cl)cc4)CC3)nc(NC3(CO)CCC3)c21]

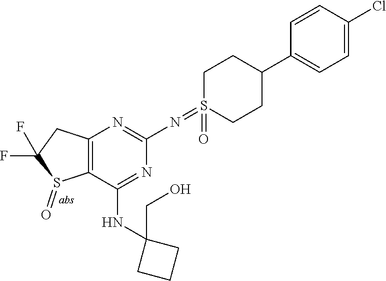 [SMILES: O=[S@@]1c2c(nc(N=S3(=O)CCC(c4ccc(Cl)cc4)CC3)nc2NC2(CO)CCC2)CC1(F)F]

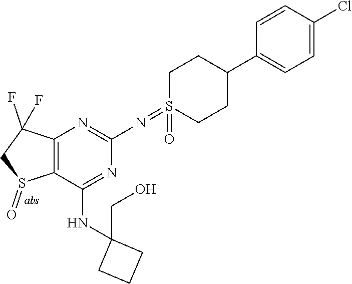 [SMILES: O=[S@]1CC(F)(F)c2nc(N=S3(=O)CCC(c4ccc(Cl)cc4)CC3)nc(NC3(CO)CCC3)c21]

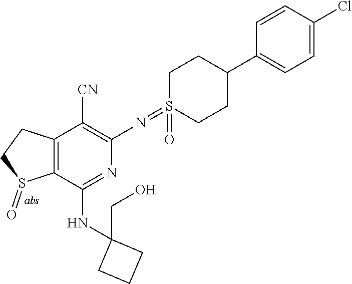 [SMILES: N#Cc1c(N=S2(=O)CCC(c3ccc(Cl)cc3)CC2)nc(NC2(CO)CCC2)c2c1CC[S@@]2=O]

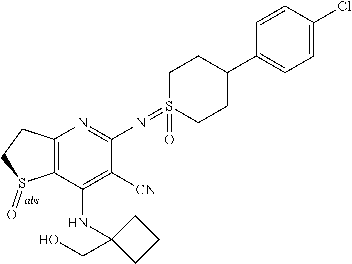 [SMILES: N#Cc1c(N=S2(=O)CCC(c3ccc(Cl)cc3)CC2)nc2c(c1NC1(CO)CCC1)[S@@](=O)CC2]

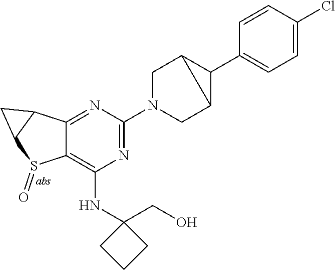

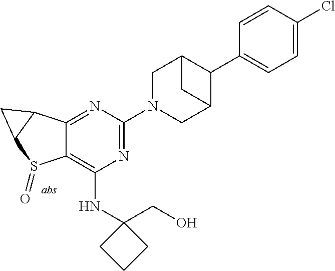

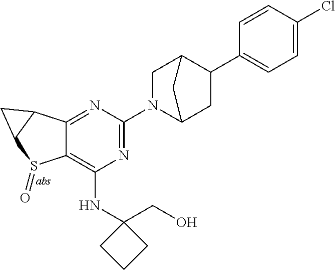

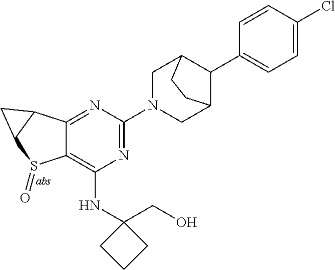

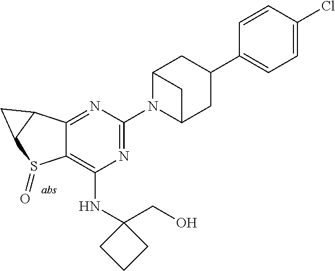

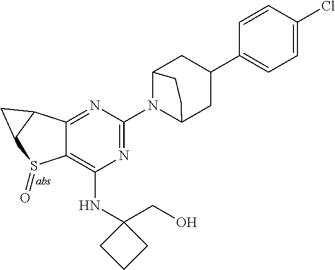

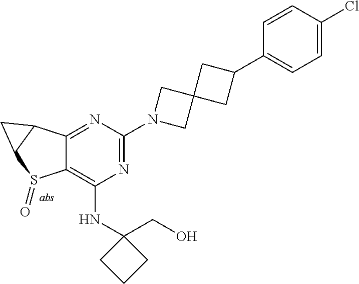

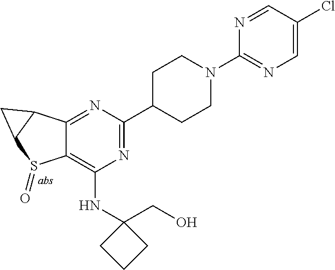

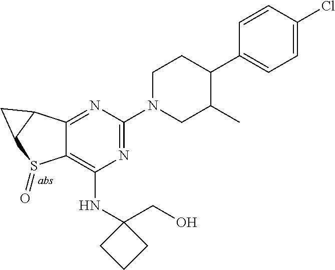

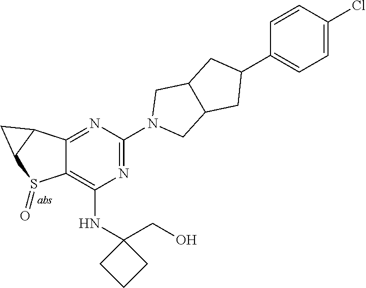

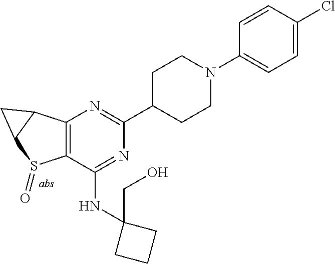

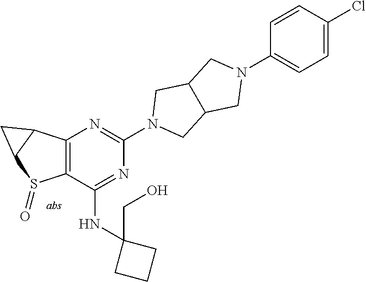

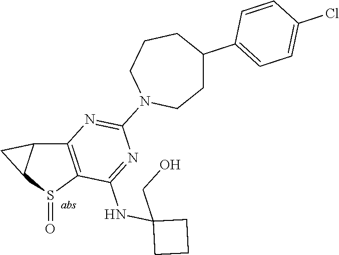

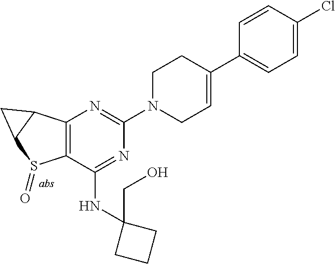

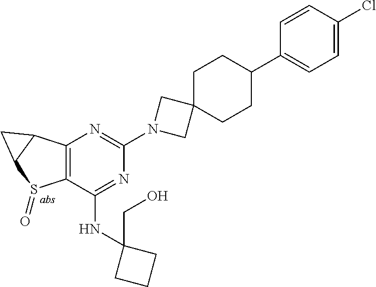

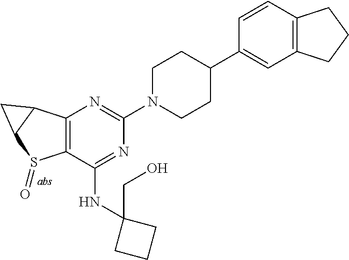

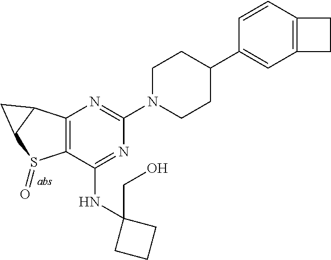

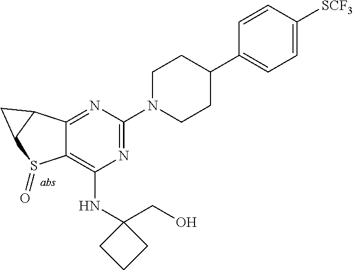

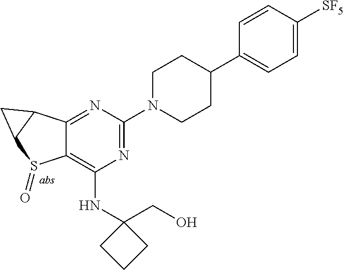

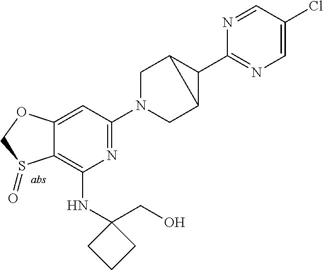 [SMILES: O=[S@]1COc2cc(N3CC4C(C3)C4c3ncc(Cl)cn3)nc(NC3(CO)CCC3)c21]

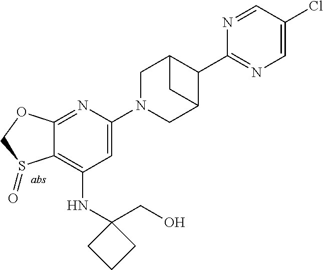 [SMILES: O=[S@]1COc2nc(N3CC4CC(C3)C4c3ncc(Cl)cn3)cc(NC3(CO)CCC3)c21]

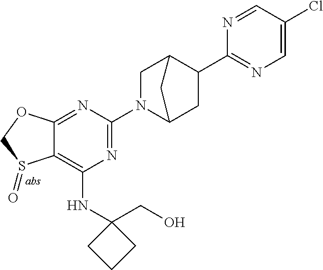 [SMILES: O=[S@]1COc2nc(N3CC4CC3CC4c3ncc(Cl)cn3)nc(NC3(CO)CCC3)c21]

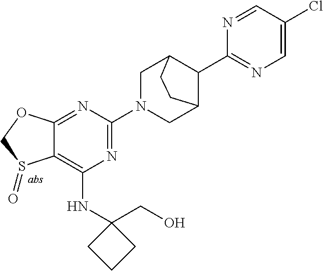 [SMILES: O=[S@]1COc2nc(N3CC4CCC(C3)C4c3ncc(Cl)cn3)nc(NC3(CO)CCC3)c21]

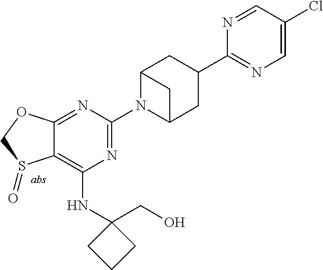 [SMILES: O=[S@]1COc2nc(N3C4CC(c5ncc(Cl)cn5)CC3C4)nc(NC3(CO)CCC3)c21]

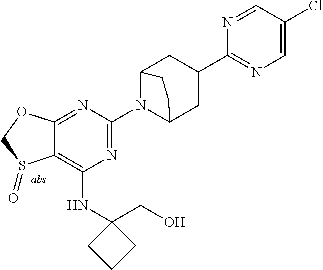 [SMILES: O=[S@]1COc2nc(N3C4CCC3CC(c3ncc(Cl)cn3)C4)nc(NC3(CO)CCC3)c21]

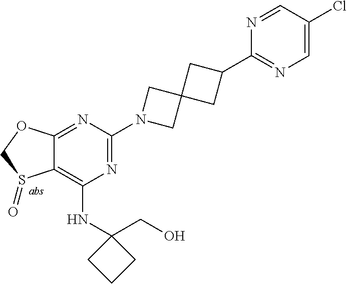 [SMILES: O=[S@]1COc2nc(N3CC4(CC(c5ncc(Cl)cn5)C4)C3)nc(NC3(CO)CCC3)c21]

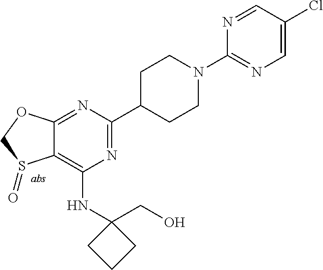 [SMILES: O=[S@]1COc2nc(C3CCN(c4ncc(Cl)cn4)CC3)nc(NC3(CO)CCC3)c21]

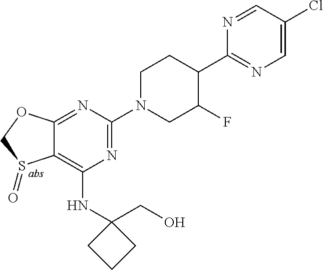 [SMILES: O=[S@]1COc2nc(N3CCC(c4ncc(Cl)cn4)C(F)C3)nc(NC3(CO)CCC3)c21]

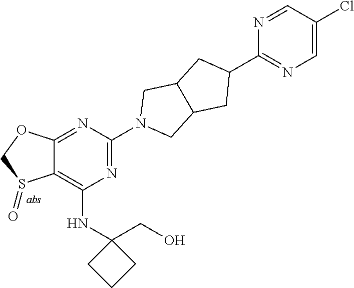 [SMILES: O=[S@]1COc2nc(N3CC4CC(c5ncc(Cl)cn5)CC4C3)nc(NC3(CO)CCC3)c21]

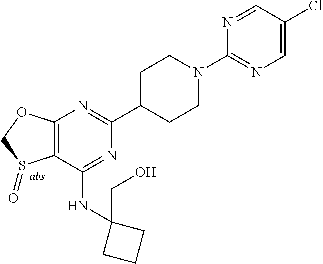 [SMILES: O=[S@]1COc2nc(C3CCN(c4ncc(Cl)cn4)CC3)nc(NC3(CO)CCC3)c21]

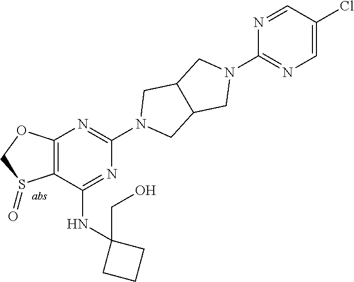 [SMILES: O=[S@]1COc2nc(N3CC4CN(c5ncc(Cl)cn5)CC4C3)nc(NC3(CO)CCC3)c21]

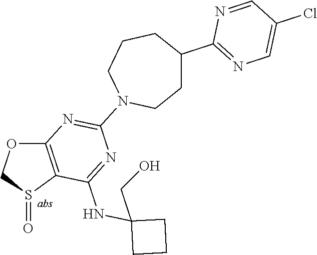 [SMILES: O=[S@]1COc2nc(N3CCCC(c4ncc(Cl)cn4)CC3)nc(NC3(CO)CCC3)c21]

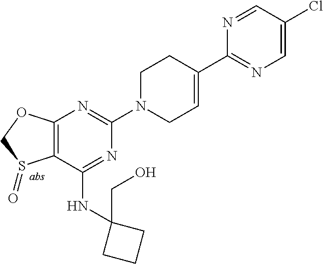 [SMILES: O=[S@]1COc2nc(N3CC=C(c4ncc(Cl)cn4)CC3)nc(NC3(CO)CCC3)c21]

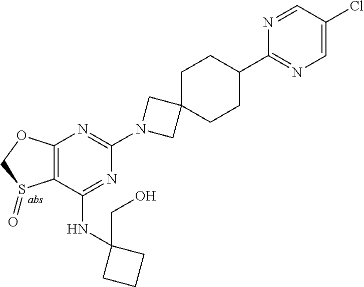 [SMILES: O=[S@]1COc2nc(N3CC4(CCC(c5ncc(Cl)cn5)CC4)C3)nc(NC3(CO)CCC3)c21]

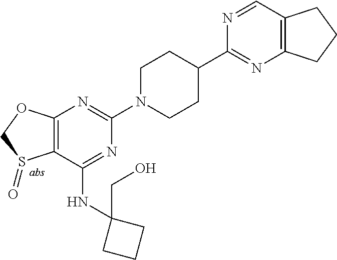 [SMILES: O=[S@]1COc2nc(N3CCC(c4ncc5c(n4)CCC5)CC3)nc(NC3(CO)CCC3)c21]

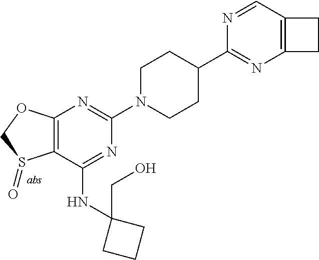 [SMILES: O=[S@]1COc2nc(N3CCC(c4ncc5c(n4)CC5)CC3)nc(NC3(CO)CCC3)c21]

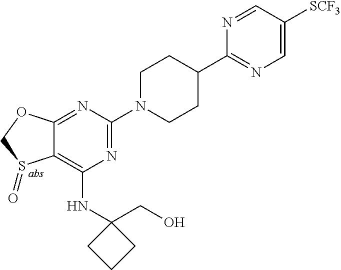

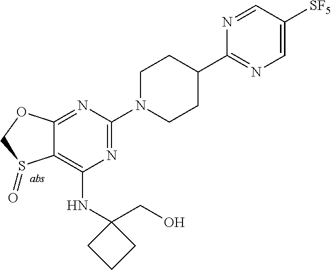

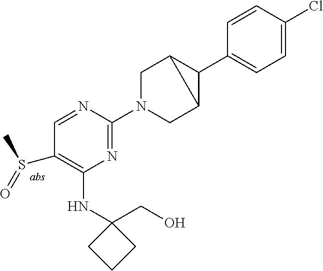 [SMILES: C[S@](=O)c1cnc(N2CC3C(C2)C3c2ccc(Cl)cc2)nc1NC1(CO)CCC1]

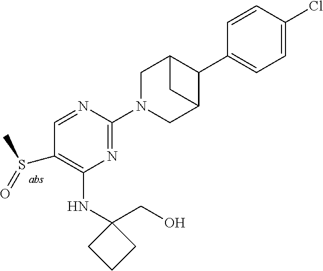 [SMILES: C[S@](=O)c1cnc(N2CC3CC(C2)C3c2ccc(Cl)cc2)nc1NC1(CO)CCC1]

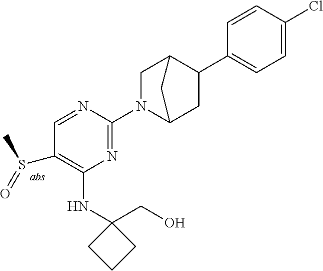 [SMILES: C[S@](=O)c1cnc(N2CC3CC2CC3c2ccc(Cl)cc2)nc1NC1(CO)CCC1]

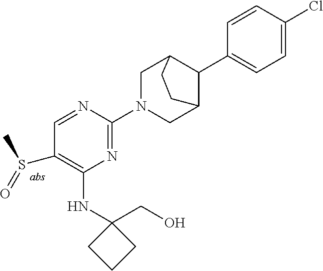 [SMILES: C[S@](=O)c1cnc(N2CC3CCC(C2)C3c2ccc(Cl)cc2)nc1NC1(CO)CCC1]

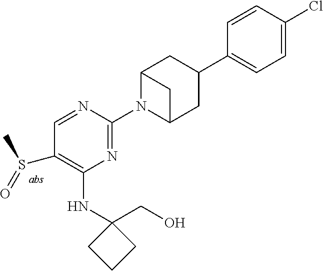 [SMILES: C[S@](=O)c1cnc(N2C3CC(c4ccc(Cl)cc4)CC2C3)nc1NC1(CO)CCC1]

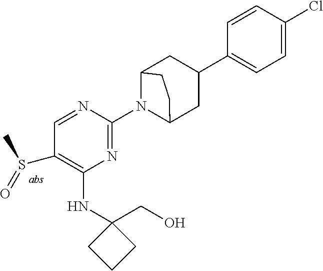 [SMILES: C[S@](=O)c1cnc(N2C3CCC2CC(c2ccc(Cl)cc2)C3)nc1NC1(CO)CCC1]

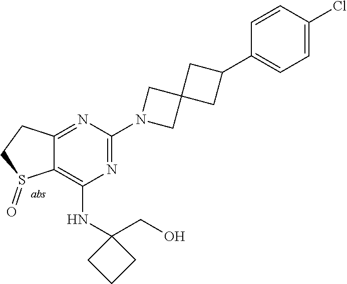 [SMILES: O=[S@]1CCc2nc(N3CC4(CC(c5ccc(Cl)cc5)C4)C3)nc(NC3(CO)CCC3)c21]

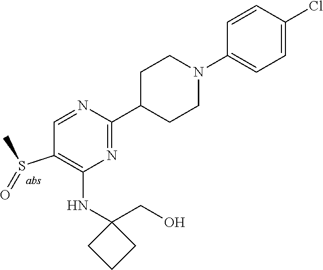 [SMILES: C[S@](=O)c1cnc(C2CCN(c3ccc(Cl)cc3)CC2)nc1NC1(CO)CCC1]

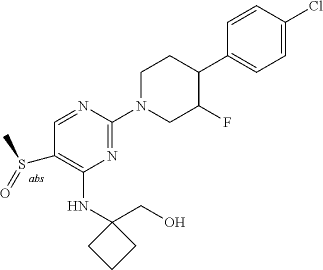 [SMILES: C[S@](=O)c1cnc(N2CCC(c3ccc(Cl)cc3)C(F)C2)nc1NC1(CO)CCC1]

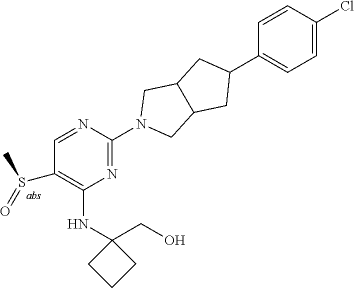 [SMILES: C[S@](=O)c1cnc(N2CC3CC(c4ccc(Cl)cc4)CC3C2)nc1NC1(CO)CCC1]

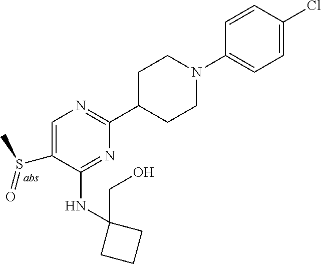 [SMILES: C[S@](=O)c1cnc(C2CCN(c3ccc(Cl)cc3)CC2)nc1NC1(CO)CCC1]

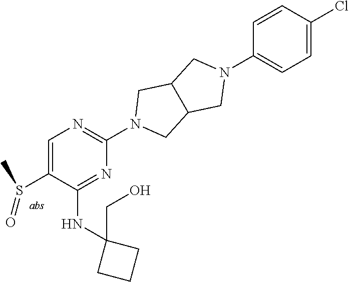 [SMILES: C[S@](=O)c1cnc(N2CC3CN(c4ccc(Cl)cc4)CC3C2)nc1NC1(CO)CCC1]

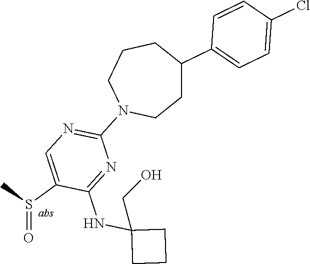 [SMILES: C[S@](=O)c1cnc(N2CCCC(c3ccc(Cl)cc3)CC2)nc1NC1(CO)CCC1]

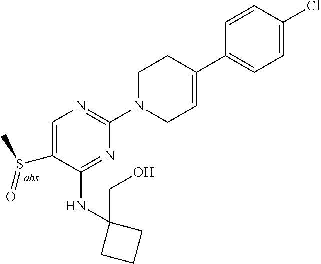 [SMILES: C[S@](=O)c1cnc(N2CC=C(c3ccc(Cl)cc3)CC2)nc1NC1(CO)CCC1]

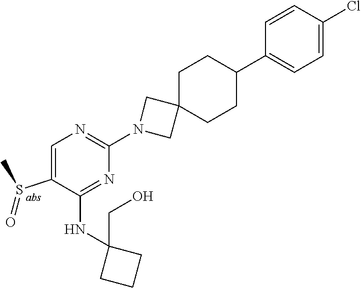 [SMILES: C[S@](=O)c1cnc(N2CC3(CCC(c4ccc(Cl)cc4)CC3)C2)nc1NC1(CO)CCC1]

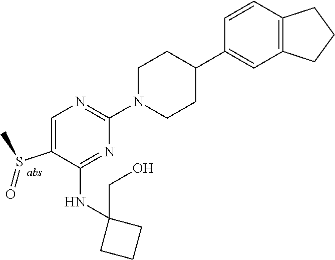 [SMILES: C[S@](=O)c1cnc(N2CCC(c3ccc4c(c3)CCC4)CC2)nc1NC1(CO)CCC1]

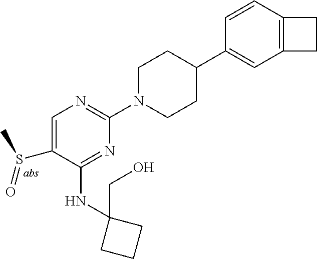 [SMILES: C[S@](=O)c1cnc(N2CCC(c3ccc4c(c3)CC4)CC2)nc1NC1(CO)CCC1]

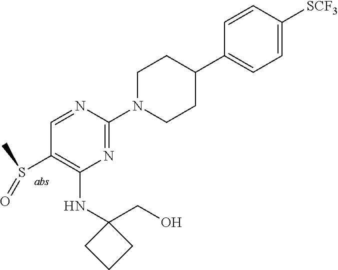

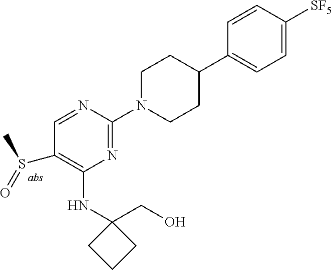

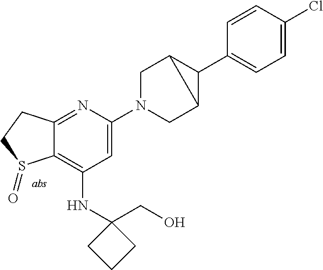 [SMILES: O=[S@]1CCc2nc(N3CC4C(C3)C4c3ccc(Cl)cc3)cc(NC3(CO)CCC3)c21]

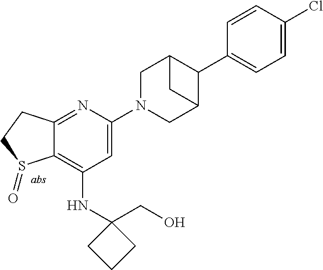 [SMILES: O=[S@]1CCc2nc(N3CC4CC(C3)C4c3ccc(Cl)cc3)cc(NC3(CO)CCC3)c21]

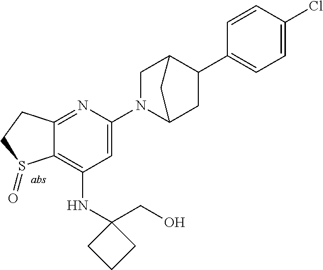 [SMILES: O=[S@]1CCc2nc(N3CC4CC3CC4c3ccc(Cl)cc3)cc(NC3(CO)CCC3)c21]

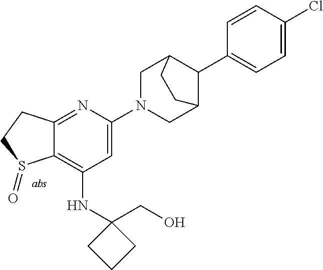 [SMILES: O=[S@]1CCc2nc(N3CC4CCC(C3)C4c3ccc(Cl)cc3)cc(NC3(CO)CCC3)c21]

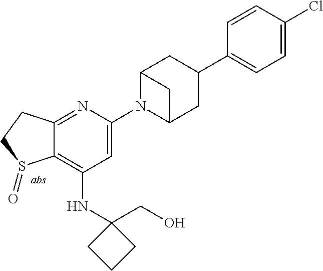 [SMILES: O=[S@]1CCc2nc(N3C4CC(c5ccc(Cl)cc5)CC3C4)cc(NC3(CO)CCC3)c21]

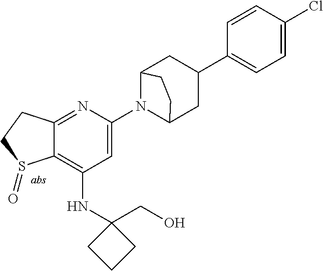 [SMILES: O=[S@]1CCc2nc(N3C4CCC3CC(c3ccc(Cl)cc3)C4)cc(NC3(CO)CCC3)c21]

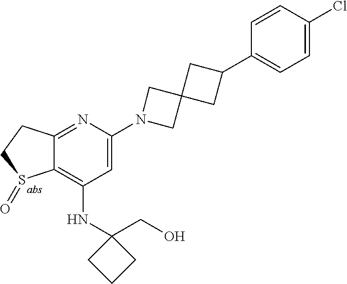 [SMILES: O=[S@]1CCc2nc(N3CC4(CC(c5ccc(Cl)cc5)C4)C3)cc(NC3(CO)CCC3)c21]

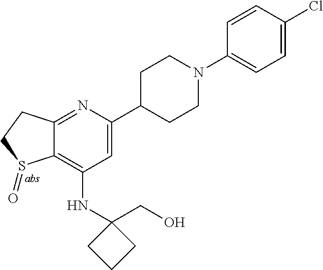 [SMILES: O=[S@]1CCc2nc(C3CCN(c4ccc(Cl)cc4)CC3)cc(NC3(CO)CCC3)c21]

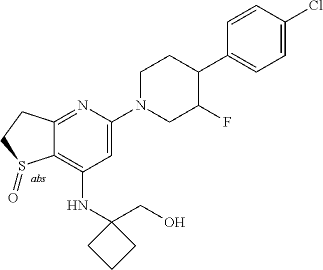 [SMILES: O=[S@]1CCc2nc(N3CCC(c4ccc(Cl)cc4)C(F)C3)cc(NC3(CO)CCC3)c21]

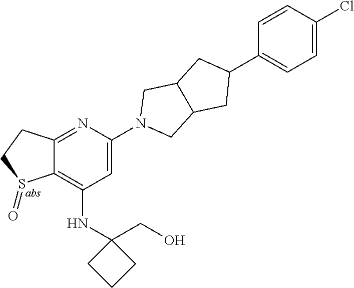 [SMILES: O=[S@]1CCc2nc(N3CC4CC(c5ccc(Cl)cc5)CC4C3)cc(NC3(CO)CCC3)c21]

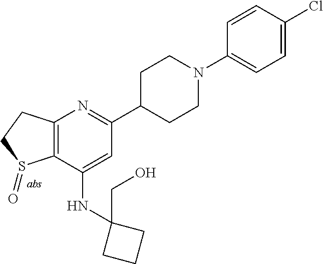 [SMILES: O=[S@]1CCc2nc(C3CCN(c4ccc(Cl)cc4)CC3)cc(NC3(CO)CCC3)c21]

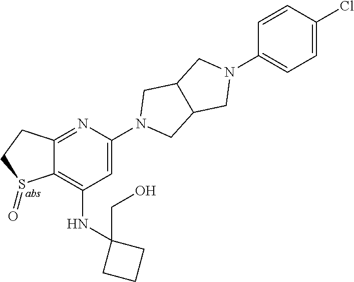 [SMILES: O=[S@]1CCc2nc(N3CC4CN(c5ccc(Cl)cc5)CC4C3)cc(NC3(CO)CCC3)c21]

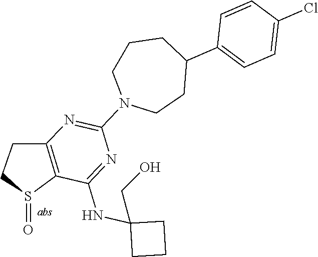 [SMILES: O=[S@]1CCc2nc(N3CCCC(c4ccc(Cl)cc4)CC3)nc(NC3(CO)CCC3)c21]

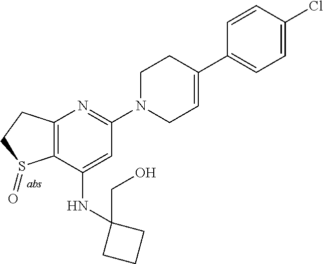 [SMILES: O=[S@]1CCc2nc(N3CC=C(c4ccc(Cl)cc4)CC3)cc(NC3(CO)CCC3)c21]

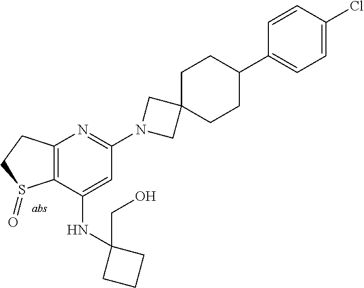 [SMILES: O=[S@]1CCc2nc(N3CC4(CCC(c5ccc(Cl)cc5)CC4)C3)cc(NC3(CO)CCC3)c21]

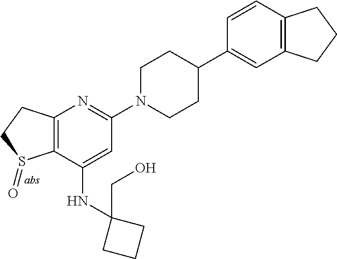 [SMILES: O=[S@]1CCc2nc(N3CCC(c4ccc5c(c4)CCC5)CC3)cc(NC3(CO)CCC3)c21]

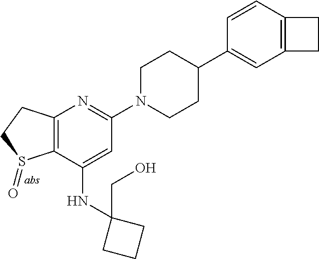 [SMILES: O=[S@]1CCc2nc(N3CCC(c4ccc5c(c4)CC5)CC3)cc(NC3(CO)CCC3)c21]

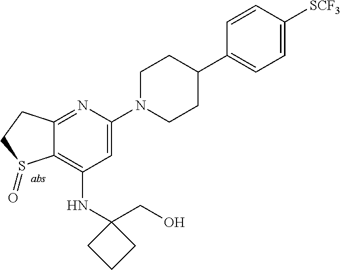

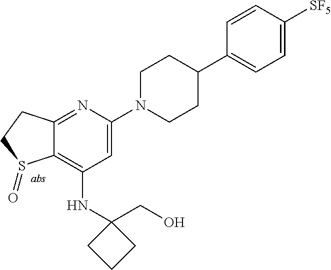

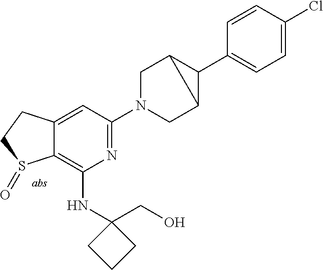 [SMILES: O=[S@]1CCc2cc(N3CC4C(C3)C4c3ccc(Cl)cc3)nc(NC3(CO)CCC3)c21]

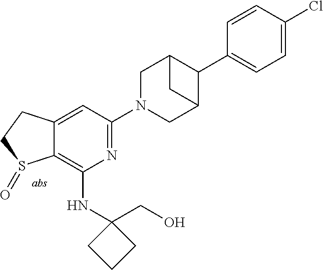 [SMILES: O=[S@]1CCc2cc(N3CC4CC(C3)C4c3ccc(Cl)cc3)nc(NC3(CO)CCC3)c21]

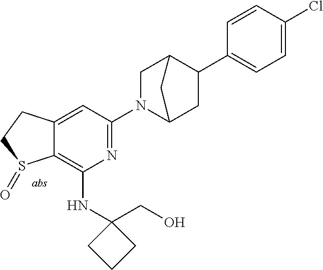 [SMILES: O=[S@]1CCc2cc(N3CC4CC3CC4c3ccc(Cl)cc3)nc(NC3(CO)CCC3)c21]

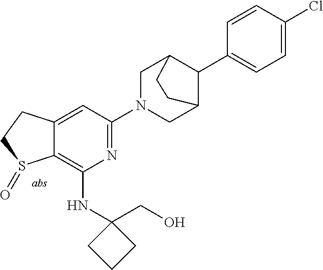 [SMILES: O=[S@]1CCc2cc(N3CC4CCC(C3)C4c3ccc(Cl)cc3)nc(NC3(CO)CCC3)c21]

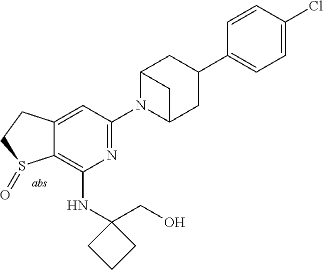 [SMILES: O=[S@]1CCc2cc(N3C4CC(c5ccc(Cl)cc5)CC3C4)nc(NC3(CO)CCC3)c21]

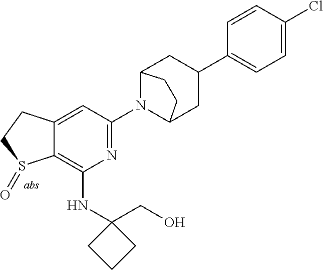 [SMILES: O=[S@]1CCc2cc(N3C4CCC3CC(c3ccc(Cl)cc3)C4)nc(NC3(CO)CCC3)c21]

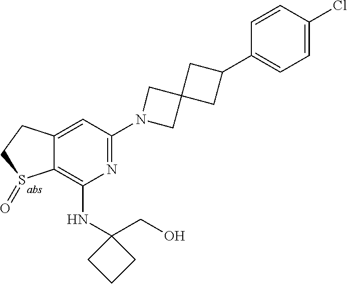 [SMILES: O=[S@]1CCc2cc(N3CC4(CC(c5ccc(Cl)cc5)C4)C3)nc(NC3(CO)CCC3)c21]

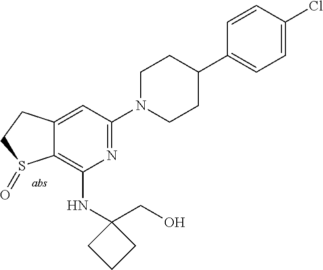 [SMILES: O=[S@]1CCc2cc(N3CCC(c4ccc(Cl)cc4)CC3)nc(NC3(CO)CCC3)c21]

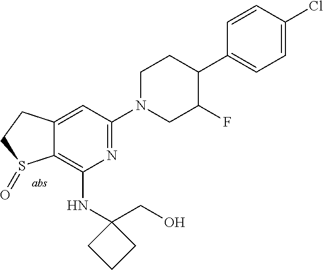 [SMILES: O=[S@]1CCc2cc(N3CCC(c4ccc(Cl)cc4)C(F)C3)nc(NC3(CO)CCC3)c21]

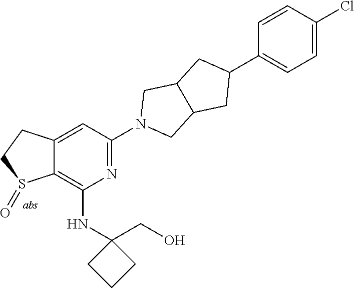 [SMILES: O=[S@]1CCc2cc(N3CC4CC(c5ccc(Cl)cc5)CC4C3)nc(NC3(CO)CCC3)c21]

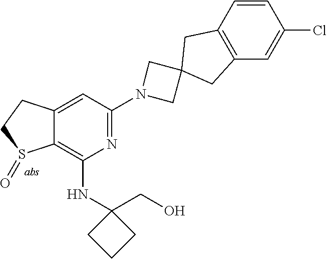 [SMILES: O=[S@]1CCc2cc(N3CC4(Cc5ccc(Cl)cc5C4)C3)nc(NC3(CO)CCC3)c21]

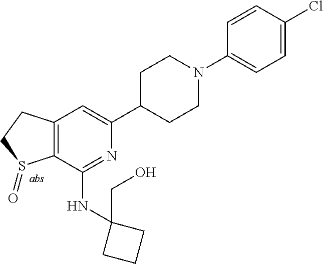 [SMILES: O=[S@]1CCc2cc(C3CCN(c4ccc(Cl)cc4)CC3)nc(NC3(CO)CCC3)c21]

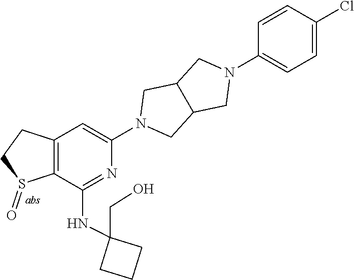 [SMILES: O=[S@]1CCc2cc(N3CC4CN(c5ccc(Cl)cc5)CC4C3)nc(NC3(CO)CCC3)c21]

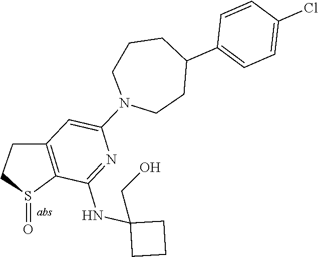 [SMILES: O=[S@]1CCc2cc(N3CCCC(c4ccc(Cl)cc4)CC3)nc(NC3(CO)CCC3)c21]

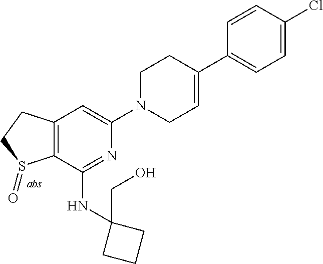 [SMILES: O=[S@]1CCc2cc(N3CC=C(c4ccc(Cl)cc4)CC3)nc(NC3(CO)CCC3)c21]

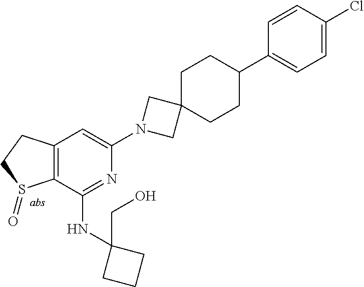 [SMILES: O=[S@]1CCc2cc(N3CC4(CCC(c5ccc(Cl)cc5)CC4)C3)nc(NC3(CO)CCC3)c21]

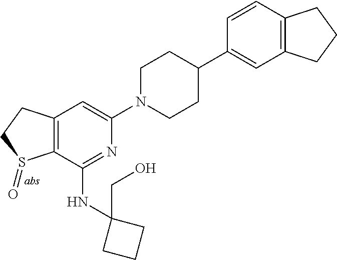 [SMILES: O=[S@]1CCc2cc(N3CCC(c4ccc5c(c4)CCC5)CC3)nc(NC3(CO)CCC3)c21]

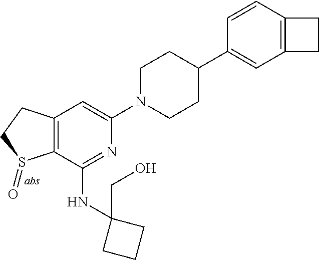 [SMILES: O=[S@]1CCc2cc(N3CCC(c4ccc5c(c4)CC5)CC3)nc(NC3(CO)CCC3)c21]

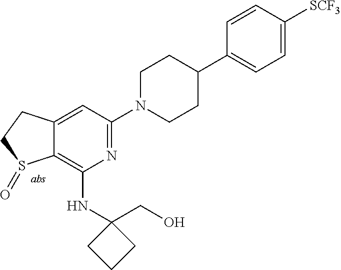

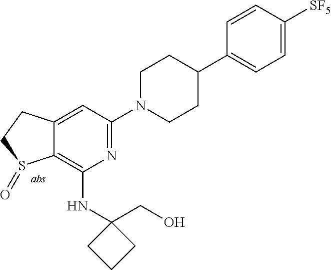

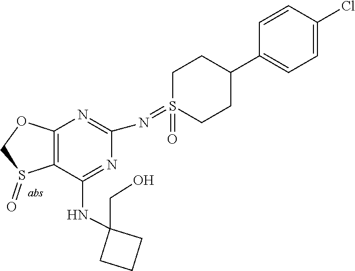 [SMILES: O=[S@]1COc2nc(N=S3(=O)CCC(c4ccc(Cl)cc4)CC3)nc(NC3(CO)CCC3)c21]

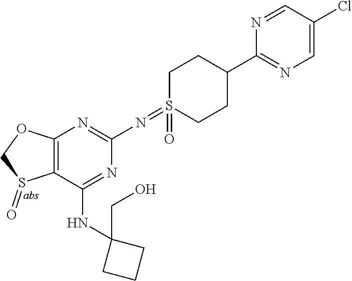 [SMILES: O=[S@]1COc2nc(N=S3(=O)CCC(c4ncc(Cl)cn4)CC3)nc(NC3(CO)CCC3)c21]

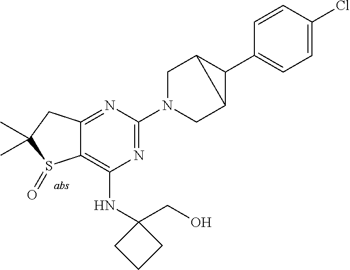 [SMILES: CC1(C)Cc2nc(N3CC4C(C3)C4c3ccc(Cl)cc3)nc(NC3(CO)CCC3)c2[S@]1=O]

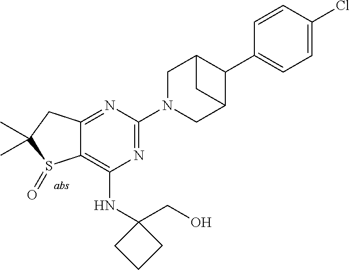 [SMILES: CC1(C)Cc2nc(N3CC4CC(C3)C4c3ccc(Cl)cc3)nc(NC3(CO)CCC3)c2[S@]1=O]

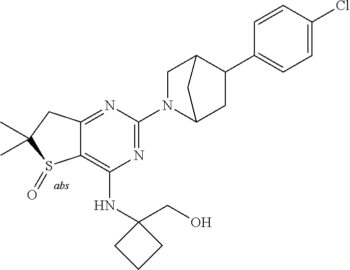 [SMILES: CC1(C)Cc2nc(N3CC4CC3CC4c3ccc(Cl)cc3)nc(NC3(CO)CCC3)c2[S@]1=O]

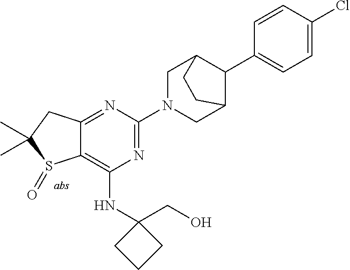 [SMILES: CC1(C)Cc2nc(N3CC4CCC(C3)C4c3ccc(Cl)cc3)nc(NC3(CO)CCC3)c2[S@]1=O]

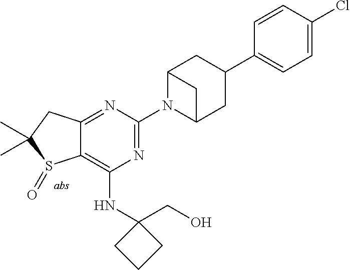 [SMILES: CC1(C)Cc2nc(N3C4CC(c5ccc(Cl)cc5)CC3C4)nc(NC3(CO)CCC3)c2[S@]1=O]

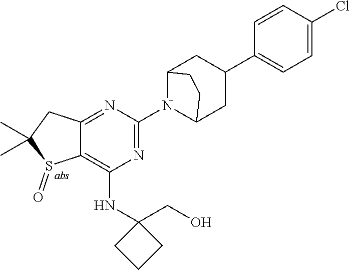 [SMILES: CC1(C)Cc2nc(N3C4CCC3CC(c3ccc(Cl)cc3)C4)nc(NC3(CO)CCC3)c2[S@]1=O]

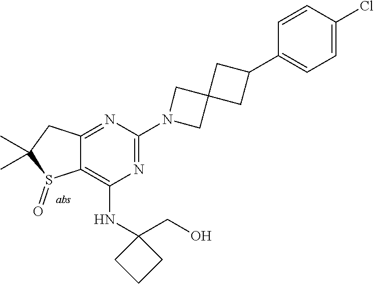 [SMILES: CC1(C)Cc2nc(N3CC4(CC(c5ccc(Cl)cc5)C4)C3)nc(NC3(CO)CCC3)c2[S@]1=O]

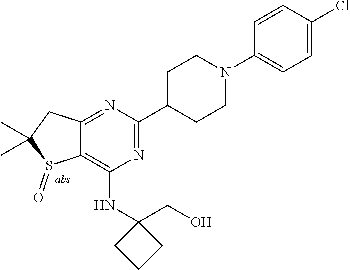 [SMILES: CC1(C)Cc2nc(C3CCN(c4ccc(Cl)cc4)CC3)nc(NC3(CO)CCC3)c2[S@]1=O]

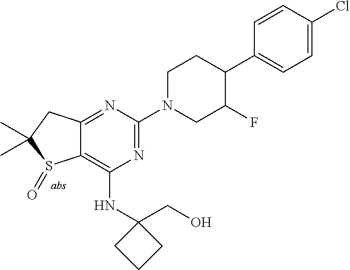 [SMILES: CC1(C)Cc2nc(N3CCC(c4ccc(Cl)cc4)C(F)C3)nc(NC3(CO)CCC3)c2[S@]1=O]

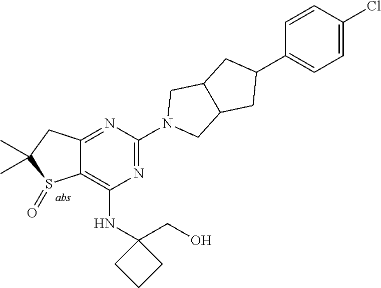 [SMILES: CC1(C)Cc2nc(N3CC4CC(c5ccc(Cl)cc5)CC4C3)nc(NC3(CO)CCC3)c2[S@]1=O]

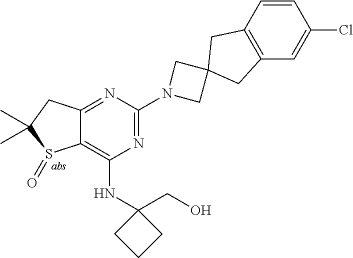 [SMILES: CC1(C)Cc2nc(N3CC4(Cc5ccc(Cl)cc5C4)C3)nc(NC3(CO)CCC3)c2[S@]1=O]

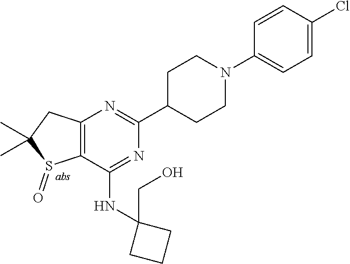 [SMILES: CC1(C)Cc2nc(C3CCN(c4ccc(Cl)cc4)CC3)nc(NC3(CO)CCC3)c2[S@]1=O]

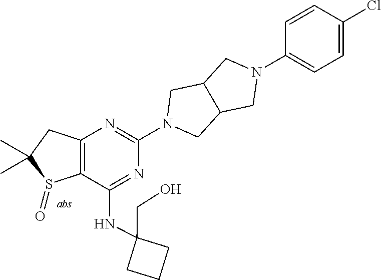 [SMILES: CC1(C)Cc2nc(N3CC4CN(c5ccc(Cl)cc5)CC4C3)nc(NC3(CO)CCC3)c2[S@]1=O]

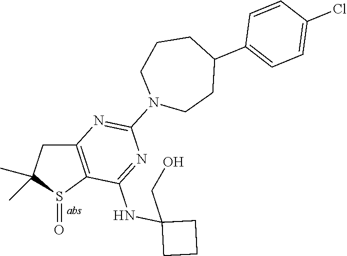 [SMILES: CC1(C)Cc2nc(N3CCCC(c4ccc(Cl)cc4)CC3)nc(NC3(CO)CCC3)c2[S@]1=O]

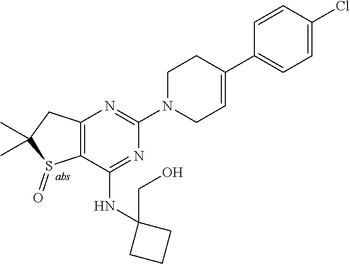 [SMILES: CC1(C)Cc2nc(N3CC=C(c4ccc(Cl)cc4)CC3)nc(NC3(CO)CCC3)c2[S@]1=O]

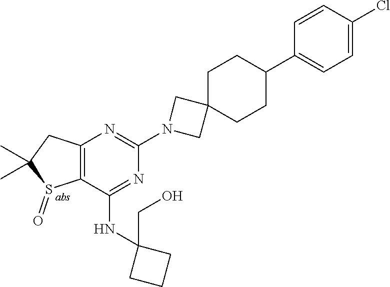 [SMILES: CC1(C)Cc2nc(N3CC4(CCC(c5ccc(Cl)cc5)CC4)C3)nc(NC3(CO)CCC3)c2[S@]1=O]

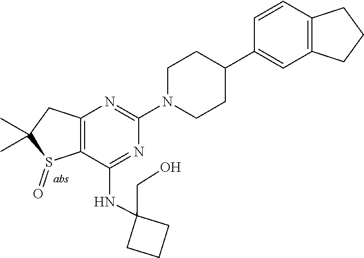 [SMILES: CC1(C)Cc2nc(N3CCC(c4ccc5c(c4)CCC5)CC3)nc(NC3(CO)CCC3)c2[S@]1=O]

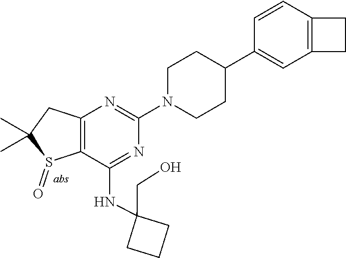 [SMILES: CC1(C)Cc2nc(N3CCC(c4ccc5c(c4)CC5)CC3)nc(NC3(CO)CCC3)c2[S@]1=O]

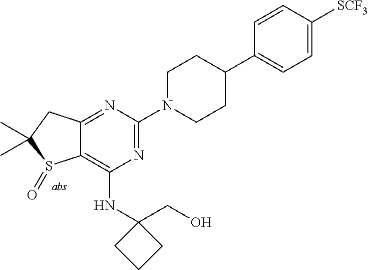

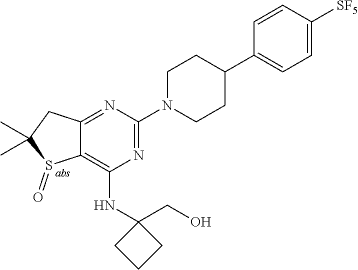

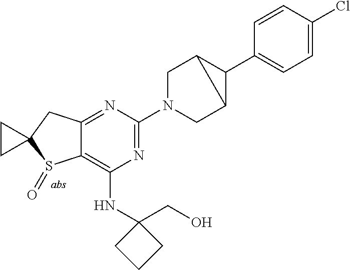 [SMILES: O=[S@@]1c2c(nc(N3CC4C(C3)C4c3ccc(Cl)cc3)nc2NC2(CO)CCC2)CC12CC2]

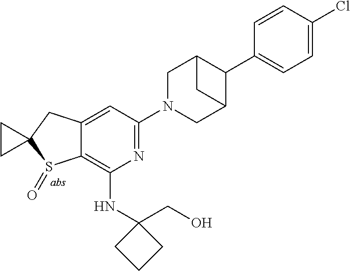 [SMILES: O=[S@@]1c2c(cc(N3CC4CC(C3)C4c3ccc(Cl)cc3)nc2NC2(CO)CCC2)CC12CC2]

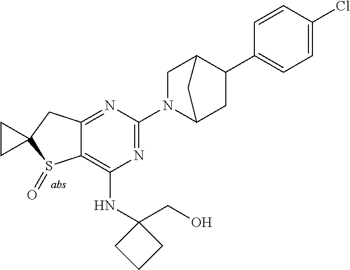 [SMILES: O=[S@@]1c2c(nc(N3CC4CC3CC4c3ccc(Cl)cc3)nc2NC2(CO)CCC2)CC12CC2]

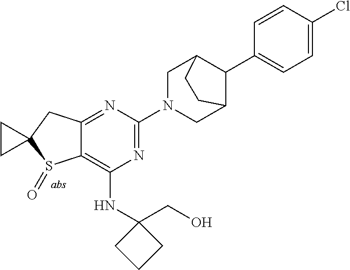 [SMILES: O=[S@@]1c2c(nc(N3CC4CCC(C3)C4c3ccc(Cl)cc3)nc2NC2(CO)CCC2)CC12CC2]

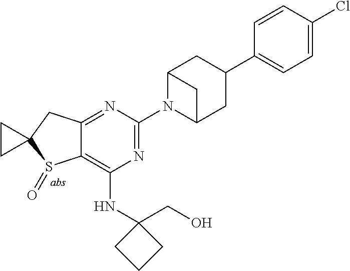 [SMILES: O=[S@@]1c2c(nc(N3C4CC(c5ccc(Cl)cc5)CC3C4)nc2NC2(CO)CCC2)CC12CC2]

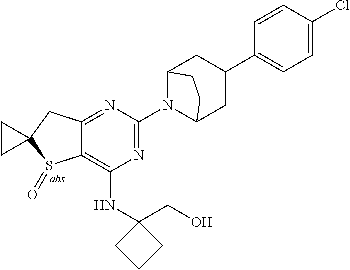 [SMILES: O=[S@@]1c2c(nc(N3C4CCC3CC(c3ccc(Cl)cc3)C4)nc2NC2(CO)CCC2)CC12CC2]

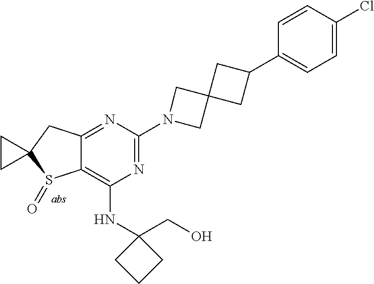 [SMILES: O=[S@@]1c2c(nc(N3CC4(CC(c5ccc(Cl)cc5)C4)C3)nc2NC2(CO)CCC2)CC12CC2]

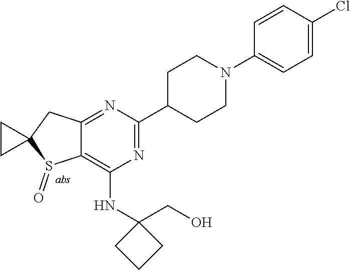 [SMILES: O=[S@@]1c2c(nc(C3CCN(c4ccc(Cl)cc4)CC3)nc2NC2(CO)CCC2)CC12CC2]

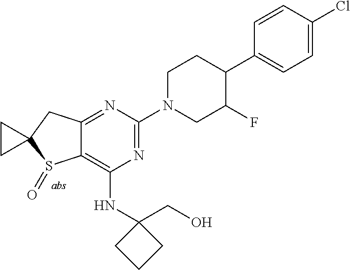 [SMILES: O=[S@@]1c2c(nc(N3CCC(c4ccc(Cl)cc4)C(F)C3)nc2NC2(CO)CCC2)CC12CC2]

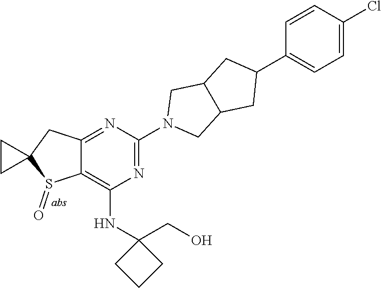 [SMILES: O=[S@@]1c2c(nc(N3CC4CC(c5ccc(Cl)cc5)CC4C3)nc2NC2(CO)CCC2)CC12CC2]

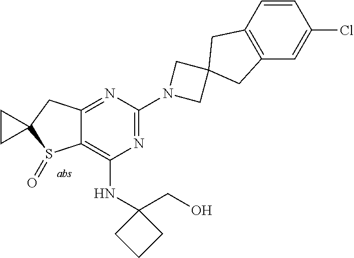 [SMILES: O=[S@@]1c2c(nc(N3CC4(Cc5ccc(Cl)cc5C4)C3)nc2NC2(CO)CCC2)CC12CC2]

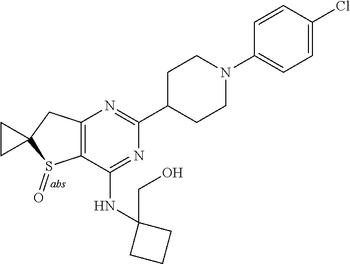 [SMILES: O=[S@@]1c2c(nc(C3CCN(c4ccc(Cl)cc4)CC3)nc2NC2(CO)CCC2)CC12CC2]

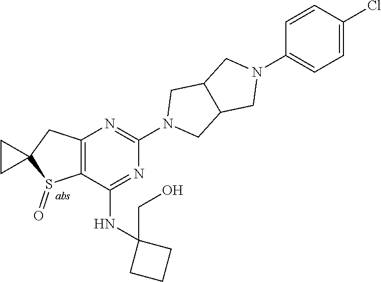 [SMILES: O=[S@@]1c2c(nc(N3CC4CN(c5ccc(Cl)cc5)CC4C3)nc2NC2(CO)CCC2)CC12CC2]

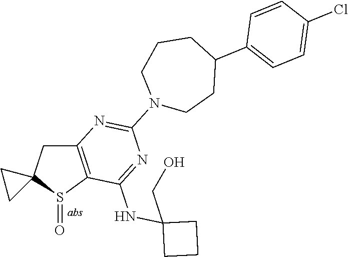 [SMILES: O=[S@@]1c2c(nc(N3CCCC(c4ccc(Cl)cc4)CC3)nc2NC2(CO)CCC2)CC12CC2]

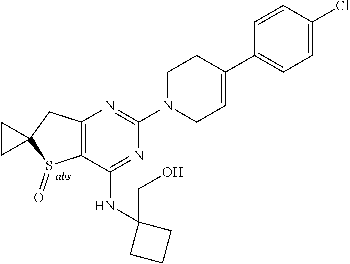 [SMILES: O=[S@@]1c2c(nc(N3CC=C(c4ccc(Cl)cc4)CC3)nc2NC2(CO)CCC2)CC12CC2]

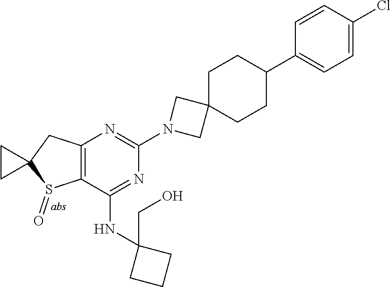 [SMILES: O=[S@@]1c2c(nc(N3CC4(CCC(c5ccc(Cl)cc5)CC4)C3)nc2NC2(CO)CCC2)CC12CC2]

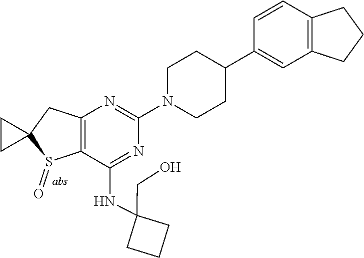 [SMILES: O=[S@@]1c2c(nc(N3CCC(c4ccc5c(c4)CCC5)CC3)nc2NC2(CO)CCC2)CC12CC2]

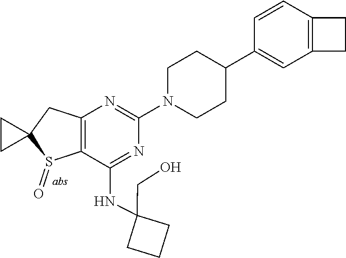 [SMILES: O=[S@@]1c2c(nc(N3CCC(c4ccc5c(c4)CC5)CC3)nc2NC2(CO)CCC2)CC12CC2]

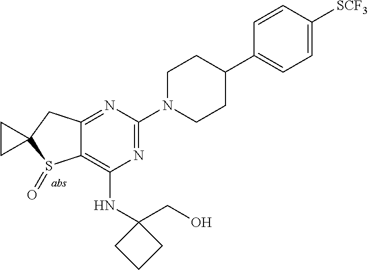

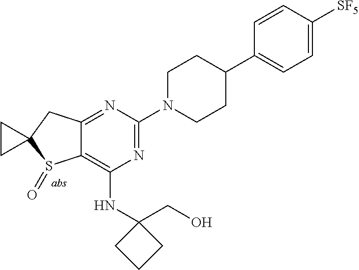

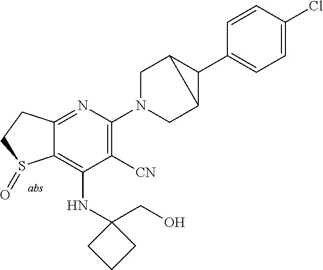 [SMILES: N#Cc1c(N2CC3C(C2)C3c2ccc(Cl)cc2)nc2c(c1NC1(CO)CCC1)[S@@](=O)CC2]

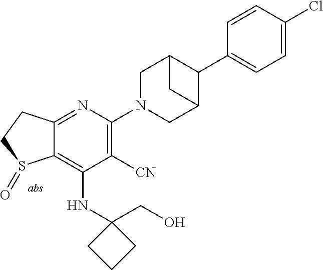 [SMILES: N#Cc1c(N2CC3CC(C2)C3c2ccc(Cl)cc2)nc2c(c1NC1(CO)CCC1)[S@@](=O)CC2]

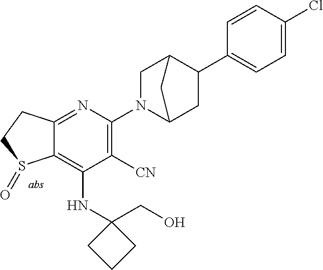 [SMILES: N#Cc1c(N2CC3CC2CC3c2ccc(Cl)cc2)nc2c(c1NC1(CO)CCC1)[S@@](=O)CC2]

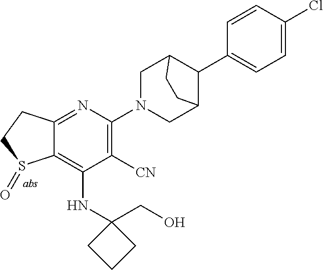 [SMILES: N#Cc1c(N2CC3CCC(C2)C3c2ccc(Cl)cc2)nc2c(c1NC1(CO)CCC1)[S@@](=O)CC2]

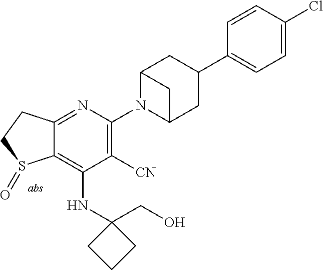 [SMILES: N#Cc1c(N2C3CC(c4ccc(Cl)cc4)CC2C3)nc2c(c1NC1(CO)CCC1)[S@@](=O)CC2]

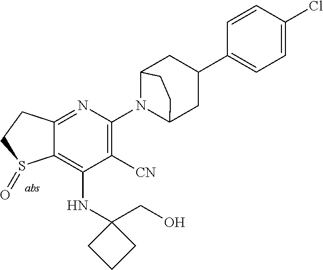 [SMILES: N#Cc1c(N2C3CCC2CC(c2ccc(Cl)cc2)C3)nc2c(c1NC1(CO)CCC1)[S@@](=O)CC2]

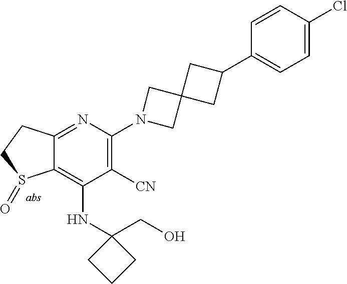 [SMILES: N#Cc1c(N2CC3(CC(c4ccc(Cl)cc4)C3)C2)nc2c(c1NC1(CO)CCC1)[S@@](=O)CC2]

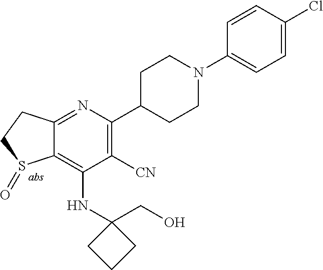 [SMILES: N#Cc1c(C2CCN(c3ccc(Cl)cc3)CC2)nc2c(c1NC1(CO)CCC1)[S@@](=O)CC2]

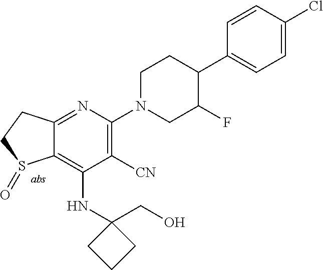 [SMILES: N#Cc1c(N2CCC(c3ccc(Cl)cc3)C(F)C2)nc2c(c1NC1(CO)CCC1)[S@@](=O)CC2]

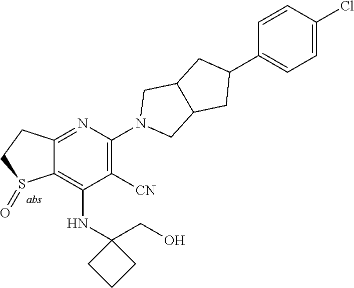 [SMILES: N#Cc1c(N2CC3CC(c4ccc(Cl)cc4)CC3C2)nc2c(c1NC1(CO)CCC1)[S@@](=O)CC2]

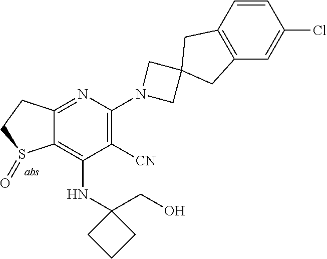 [SMILES: N#Cc1c(N2CC3(Cc4ccc(Cl)cc4C3)C2)nc2c(c1NC1(CO)CCC1)[S@@](=O)CC2]

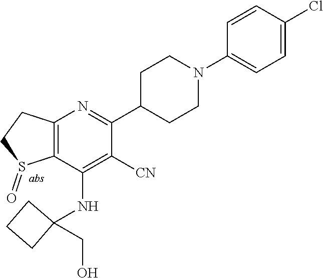 [SMILES: N#Cc1c(C2CCN(c3ccc(Cl)cc3)CC2)nc2c(c1NC1(CO)CCC1)[S@@](=O)CC2]

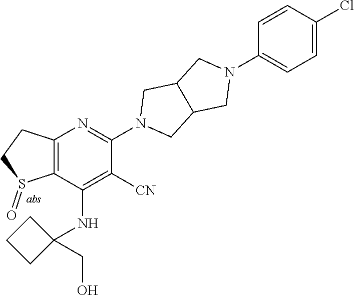 [SMILES: N#Cc1c(N2CC3CN(c4ccc(Cl)cc4)CC3C2)nc2c(c1NC1(CO)CCC1)[S@@](=O)CC2]

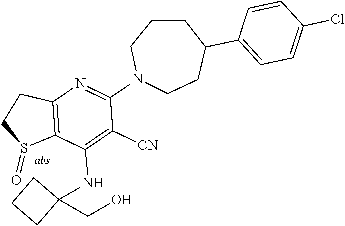 [SMILES: N#Cc1c(N2CCCC(c3ccc(Cl)cc3)CC2)nc2c(c1NC1(CO)CCC1)[S@@](=O)CC2]

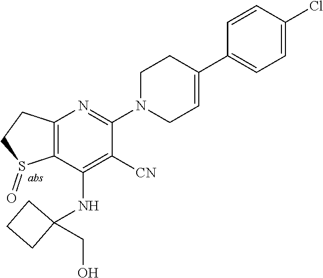 [SMILES: N#Cc1c(N2CC=C(c3ccc(Cl)cc3)CC2)nc2c(c1NC1(CO)CCC1)[S@@](=O)CC2]

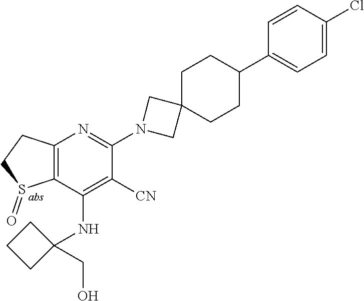 [SMILES: N#Cc1c(N2CC3(CCC(c4ccc(Cl)cc4)CC3)C2)nc2c(c1NC1(CO)CCC1)[S@@](=O)CC2]

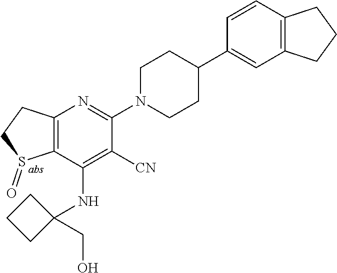 [SMILES: N#Cc1c(N2CCC(c3ccc4c(c3)CCC4)CC2)nc2c(c1NC1(CO)CCC1)[S@@](=O)CC2]

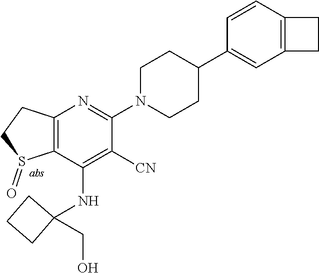 [SMILES: N#Cc1c(N2CCC(c3ccc4c(c3)CC4)CC2)nc2c(c1NC1(CO)CCC1)[S@@](=O)CC2]

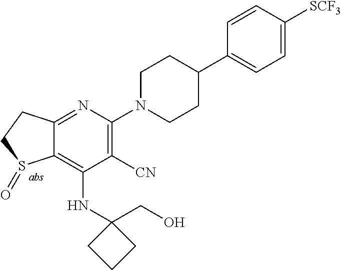

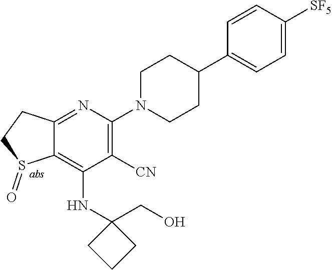

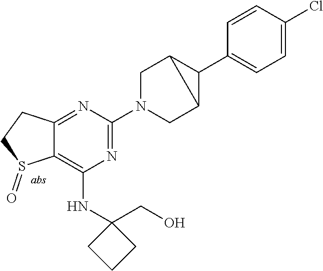 [SMILES: O=[S@]1CCc2nc(N3CC4C(C3)C4c3ccc(Cl)cc3)nc(NC3(CO)CCC3)c21]

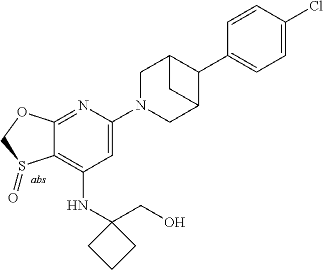 [SMILES: O=[S@]1COc2nc(N3CC4CC(C3)C4c3ccc(Cl)cc3)cc(NC3(CO)CCC3)c21]

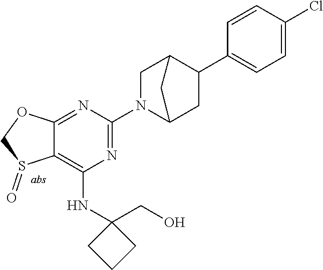 [SMILES: O=[S@]1COc2nc(N3CC4CC3CC4c3ccc(Cl)cc3)nc(NC3(CO)CCC3)c21]

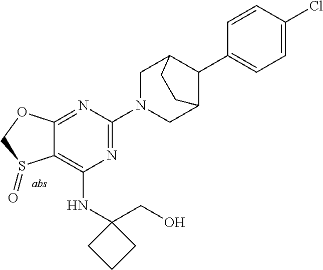 [SMILES: O=[S@]1COc2nc(N3CC4CCC(C3)C4c3ccc(Cl)cc3)nc(NC3(CO)CCC3)c21]

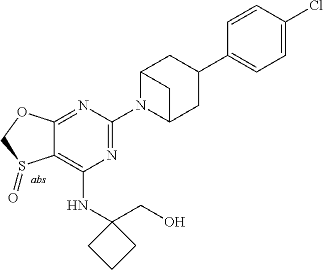 [SMILES: O=[S@]1COc2nc(N3C4CC(c5ccc(Cl)cc5)CC3C4)nc(NC3(CO)CCC3)c21]

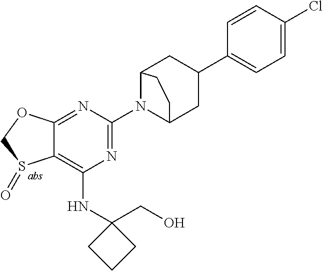 [SMILES: O=[S@]1COc2nc(N3C4CCC3CC(c3ccc(Cl)cc3)C4)nc(NC3(CO)CCC3)c21]

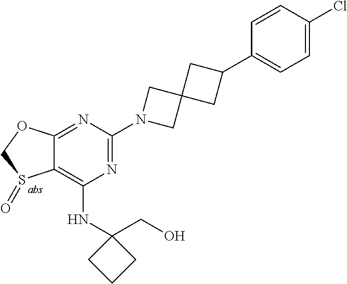 [SMILES: O=[S@]1COc2nc(N3CC4(CC(c5ccc(Cl)cc5)C4)C3)nc(NC3(CO)CCC3)c21]

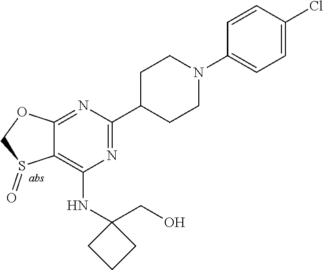 [SMILES: O=[S@]1COc2nc(C3CCN(c4ccc(Cl)cc4)CC3)nc(NC3(CO)CCC3)c21]

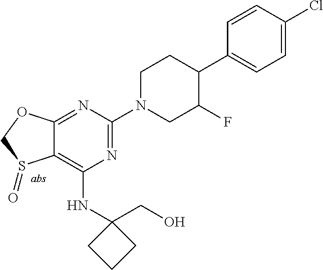 [SMILES: O=[S@]1COc2nc(N3CCC(c4ccc(Cl)cc4)C(F)C3)nc(NC3(CO)CCC3)c21]

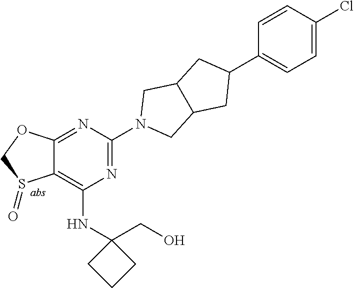 [SMILES: O=[S@]1COc2nc(N3CC4CC(c5ccc(Cl)cc5)CC4C3)nc(NC3(CO)CCC3)c21]

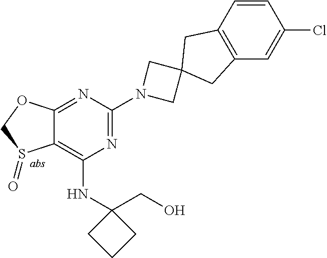 [SMILES: O=[S@]1COc2nc(N3CC4(Cc5ccc(Cl)cc5C4)C3)nc(NC3(CO)CCC3)c21]

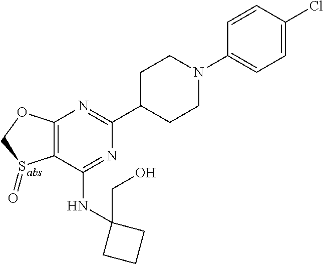 [SMILES: O=[S@]1COc2nc(C3CCN(c4ccc(Cl)cc4)CC3)nc(NC3(CO)CCC3)c21]

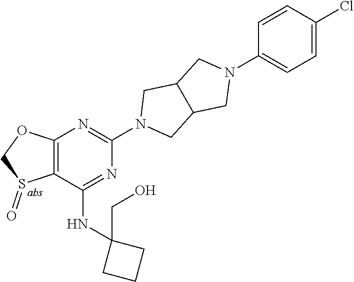 [SMILES: O=[S@]1COc2nc(N3CC4CN(c5ccc(Cl)cc5)CC4C3)nc(NC3(CO)CCC3)c21]

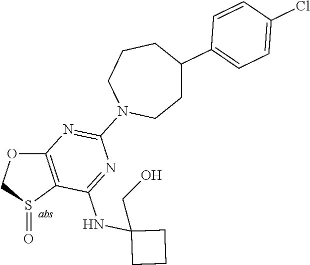 [SMILES: O=[S@]1COc2nc(N3CCCC(c4ccc(Cl)cc4)CC3)nc(NC3(CO)CCC3)c21]

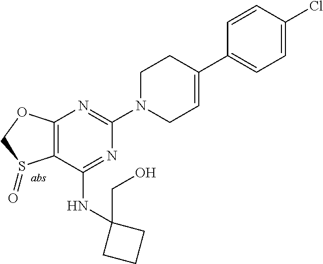 [SMILES: O=[S@]1COc2nc(N3CC=C(c4ccc(Cl)cc4)CC3)nc(NC3(CO)CCC3)c21]

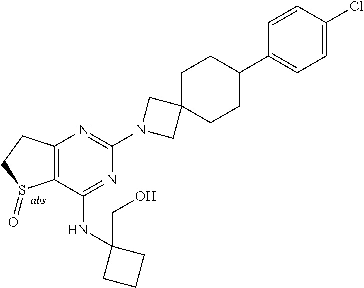 [SMILES: O=[S@]1CCc2nc(N3CC4(CCC(c5ccc(Cl)cc5)CC4)C3)nc(NC3(CO)CCC3)c21]

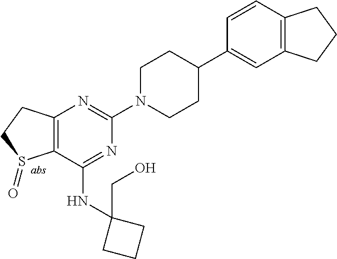 [SMILES: O=[S@]1CCc2nc(N3CCC(c4ccc5c(c4)CCC5)CC3)nc(NC3(CO)CCC3)c21]

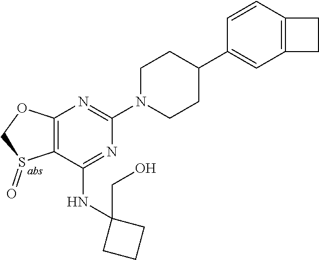 [SMILES: O=[S@]1COc2nc(N3CCC(c4ccc5c(c4)CC5)CC3)nc(NC3(CO)CCC3)c21]

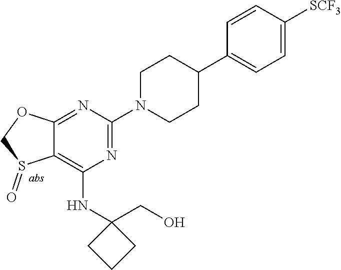

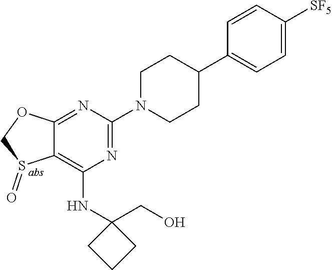

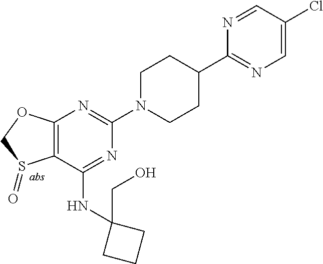 [SMILES: O=[S@]1COc2nc(N3CCC(c4ncc(Cl)cn4)CC3)nc(NC3(CO)CCC3)c21]

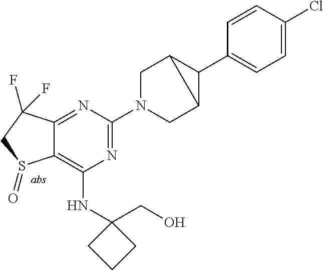 [SMILES: O=[S@]1CC(F)(F)c2nc(N3CC4C(C3)C4c3ccc(Cl)cc3)nc(NC3(CO)CCC3)c21]

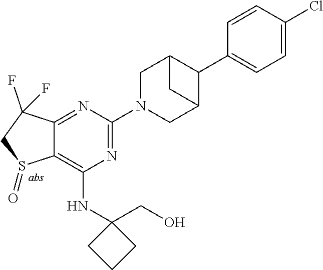 [SMILES: O=[S@]1CC(F)(F)c2nc(N3CC4CC(C3)C4c3ccc(Cl)cc3)nc(NC3(CO)CCC3)c21]

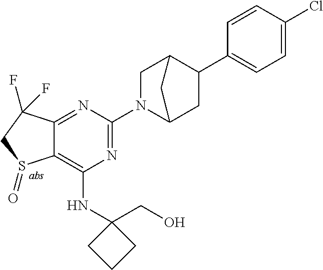 [SMILES: O=[S@]1CC(F)(F)c2nc(N3CC4CC3CC4c3ccc(Cl)cc3)nc(NC3(CO)CCC3)c21]

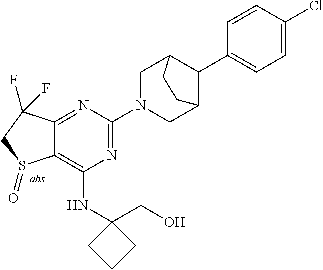 [SMILES: O=[S@]1CC(F)(F)c2nc(N3CC4CCC(C3)C4c3ccc(Cl)cc3)nc(NC3(CO)CCC3)c21]

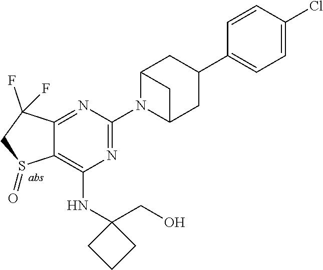 [SMILES: O=[S@]1CC(F)(F)c2nc(N3C4CC(c5ccc(Cl)cc5)CC3C4)nc(NC3(CO)CCC3)c21]

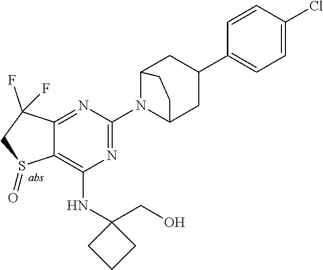 [SMILES: O=[S@]1CC(F)(F)c2nc(N3C4CCC3CC(c3ccc(Cl)cc3)C4)nc(NC3(CO)CCC3)c21]

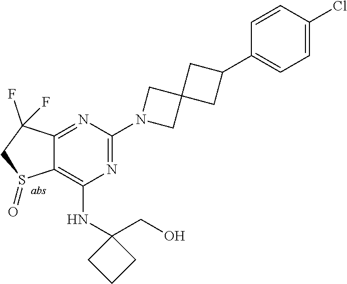 [SMILES: O=[S@]1CC(F)(F)c2nc(N3CC4(CC(c5ccc(Cl)cc5)C4)C3)nc(NC3(CO)CCC3)c21]

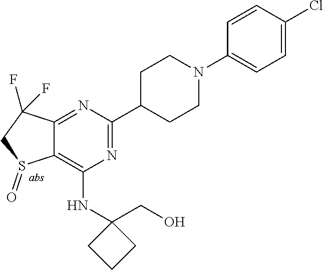 [SMILES: O=[S@]1CC(F)(F)c2nc(C3CCN(c4ccc(Cl)cc4)CC3)nc(NC3(CO)CCC3)c21]

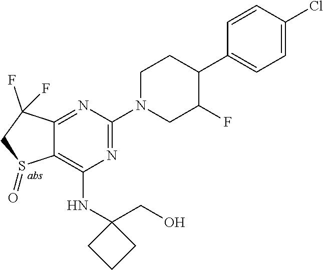 [SMILES: O=[S@]1CC(F)(F)c2nc(N3CCC(c4ccc(Cl)cc4)C(F)C3)nc(NC3(CO)CCC3)c21]

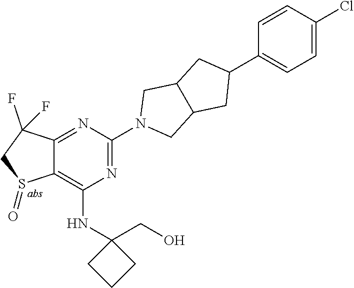 [SMILES: O=[S@]1CC(F)(F)c2nc(N3CC4CC(c5ccc(Cl)cc5)CC4C3)nc(NC3(CO)CCC3)c21]

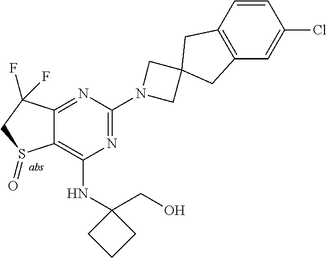 [SMILES: O=[S@]1CC(F)(F)c2nc(N3CC4(Cc5ccc(Cl)cc5C4)C3)nc(NC3(CO)CCC3)c21]

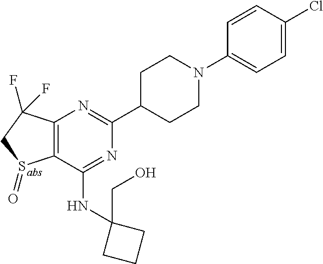 [SMILES: O=[S@]1CC(F)(F)c2nc(C3CCN(c4ccc(Cl)cc4)CC3)nc(NC3(CO)CCC3)c21]

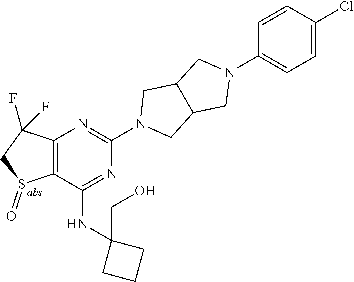 [SMILES: O=[S@]1CC(F)(F)c2nc(N3CC4CN(c5ccc(Cl)cc5)CC4C3)nc(NC3(CO)CCC3)c21]

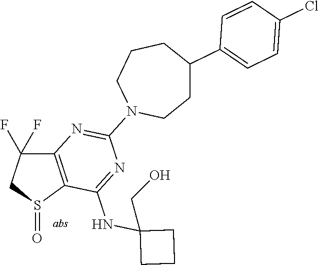 [SMILES: O=[S@]1CC(F)(F)c2nc(N3CCCC(c4ccc(Cl)cc4)CC3)nc(NC3(CO)CCC3)c21]

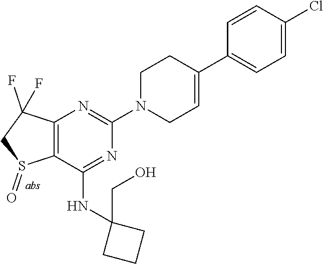 [SMILES: O=[S@]1CC(F)(F)c2nc(N3CC=C(c4ccc(Cl)cc4)CC3)nc(NC3(CO)CCC3)c21]

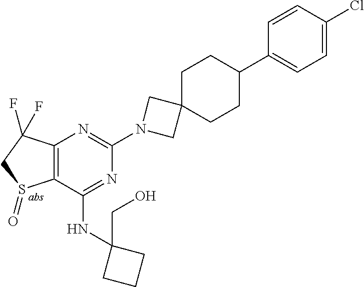 [SMILES: O=[S@]1CC(F)(F)c2nc(N3CC4(CCC(c5ccc(Cl)cc5)CC4)C3)nc(NC3(CO)CCC3)c21]

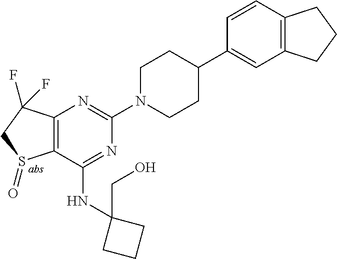 [SMILES: O=[S@]1CC(F)(F)c2nc(N3CCC(c4ccc5c(c4)CCC5)CC3)nc(NC3(CO)CCC3)c21]

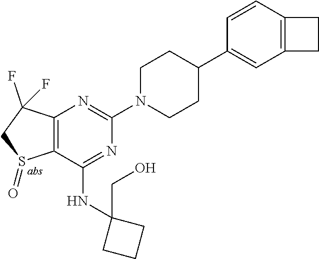 [SMILES: O=[S@]1CC(F)(F)c2nc(N3CCC(c4ccc5c(c4)CC5)CC3)nc(NC3(CO)CCC3)c21]

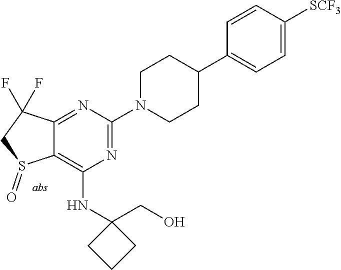

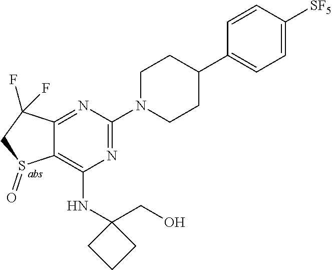

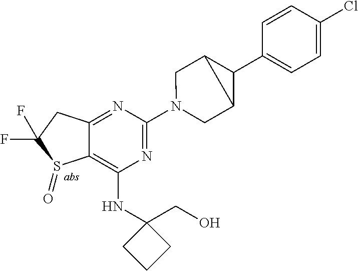 [SMILES: O=[S@@]1c2c(nc(N3CC4C(C3)C4c3ccc(Cl)cc3)nc2NC2(CO)CCC2)CC1(F)F]

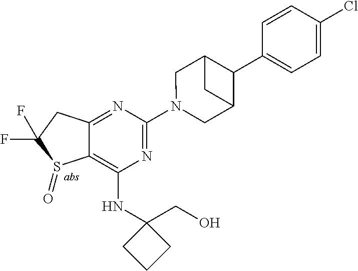 [SMILES: O=[S@@]1c2c(nc(N3CC4CC(C3)C4c3ccc(Cl)cc3)nc2NC2(CO)CCC2)CC1(F)F]

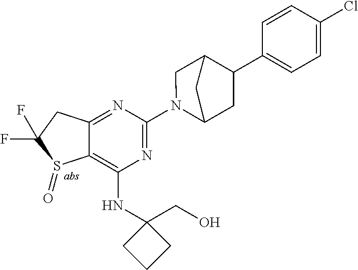 [SMILES: O=[S@@]1c2c(nc(N3CC4CC3CC4c3ccc(Cl)cc3)nc2NC2(CO)CCC2)CC1(F)F]

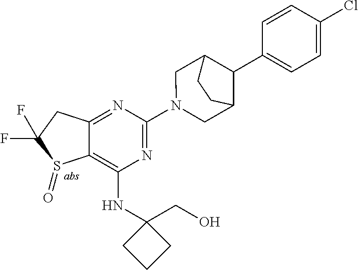 [SMILES: O=[S@@]1c2c(nc(N3CC4CCC(C3)C4c3ccc(Cl)cc3)nc2NC2(CO)CCC2)CC1(F)F]

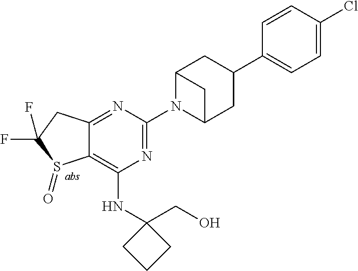 [SMILES: O=[S@@]1c2c(nc(N3C4CC(c5ccc(Cl)cc5)CC3C4)nc2NC2(CO)CCC2)CC1(F)F]

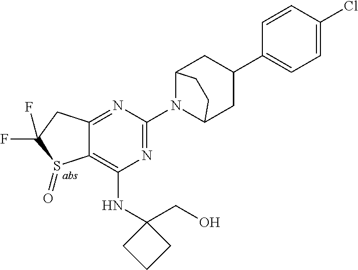 [SMILES: O=[S@@]1c2c(nc(N3C4CCC3CC(c3ccc(Cl)cc3)C4)nc2NC2(CO)CCC2)CC1(F)F]

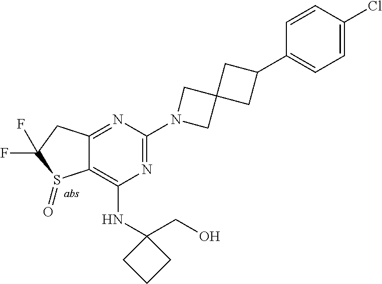 [SMILES: O=[S@@]1c2c(nc(N3CC4(CC(c5ccc(Cl)cc5)C4)C3)nc2NC2(CO)CCC2)CC1(F)F]

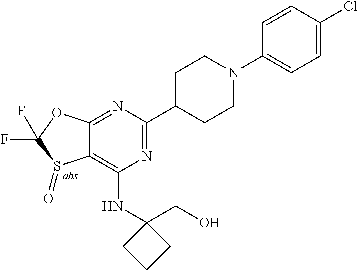 [SMILES: O=[S@@]1c2c(NC3(CO)CCC3)nc(C3CCN(c4ccc(Cl)cc4)CC3)nc2OC1(F)F]

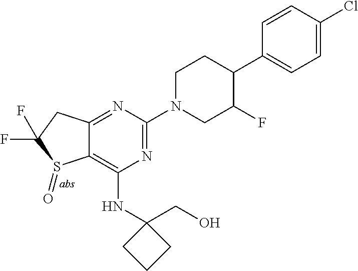 [SMILES: O=[S@@]1c2c(nc(N3CCC(c4ccc(Cl)cc4)C(F)C3)nc2NC2(CO)CCC2)CC1(F)F]

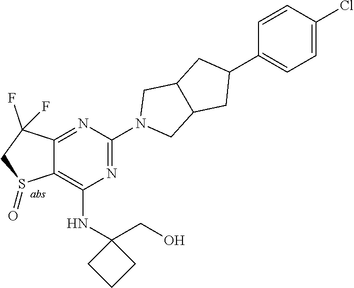 [SMILES: O=[S@]1CC(F)(F)c2nc(N3CC4CC(c5ccc(Cl)cc5)CC4C3)nc(NC3(CO)CCC3)c21]

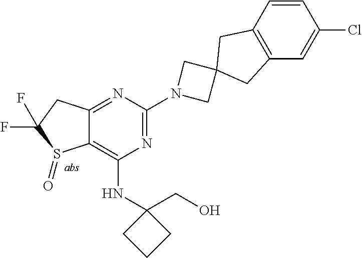 [SMILES: O=[S@@]1c2c(nc(N3CC4(Cc5ccc(Cl)cc5C4)C3)nc2NC2(CO)CCC2)CC1(F)F]

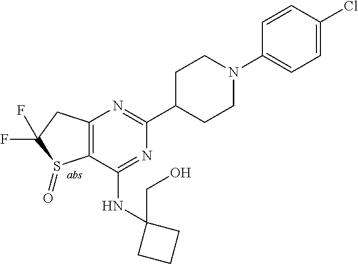 [SMILES: O=[S@@]1c2c(nc(C3CCN(c4ccc(Cl)cc4)CC3)nc2NC2(CO)CCC2)CC1(F)F]

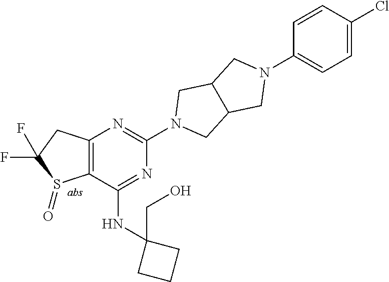 [SMILES: O=[S@@]1c2c(nc(N3CC4CN(c5ccc(Cl)cc5)CC4C3)nc2NC2(CO)CCC2)CC1(F)F]

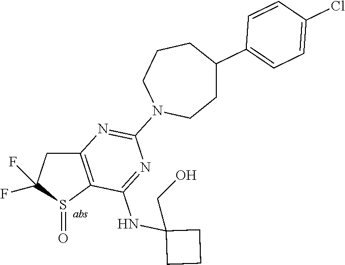 [SMILES: O=[S@@]1c2c(nc(N3CCCC(c4ccc(Cl)cc4)CC3)nc2NC2(CO)CCC2)CC1(F)F]

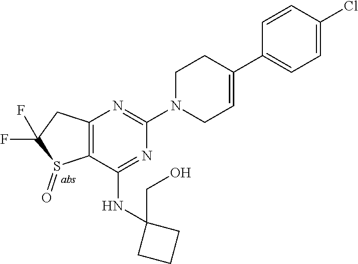 [SMILES: O=[S@@]1c2c(nc(N3CC=C(c4ccc(Cl)cc4)CC3)nc2NC2(CO)CCC2)CC1(F)F]

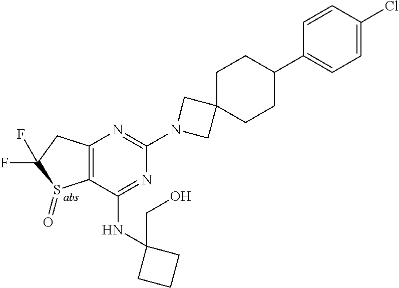 [SMILES: O=[S@@]1c2c(nc(N3CC4(CCC(c5ccc(Cl)cc5)CC4)C3)nc2NC2(CO)CCC2)CC1(F)F]

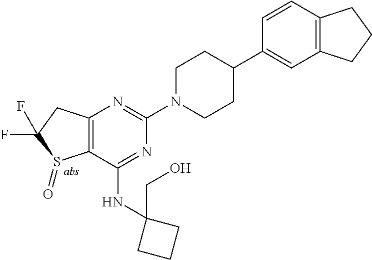 [SMILES: O=[S@@]1c2c(nc(N3CCC(c4ccc5c(c4)CCC5)CC3)nc2NC2(CO)CCC2)CC1(F)F]

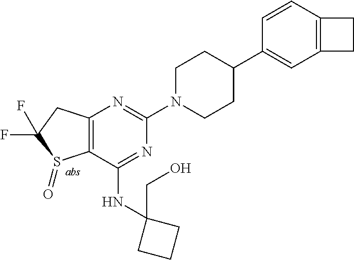 [SMILES: O=[S@@]1c2c(nc(N3CCC(c4ccc5c(c4)CC5)CC3)nc2NC2(CO)CCC2)CC1(F)F]

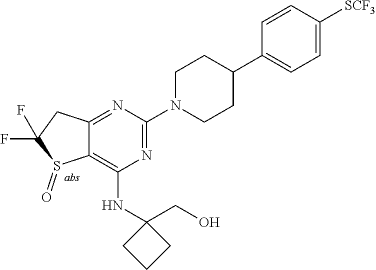

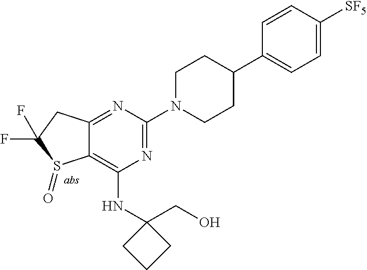

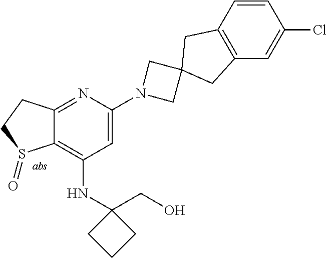 [SMILES: O=[S@]1CCc2nc(N3CC4(Cc5ccc(Cl)cc5C4)C3)cc(NC3(CO)CCC3)c21]

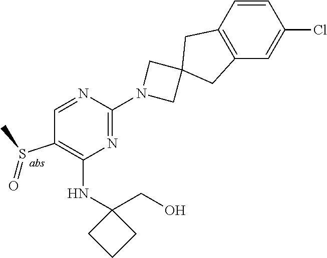 [SMILES: C[S@](=O)c1cnc(N2CC3(Cc4ccc(Cl)cc4C3)C2)nc1NC1(CO)CCC1]

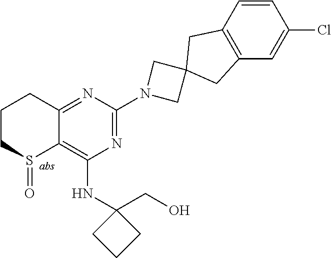 [SMILES: O=[S@]1CCCc2nc(N3CC4(Cc5ccc(Cl)cc5C4)C3)nc(NC3(CO)CCC3)c21]

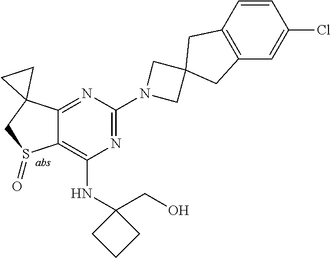 [SMILES: O=[S@]1CC2(CC2)c2nc(N3CC4(Cc5ccc(Cl)cc5C4)C3)nc(NC3(CO)CCC3)c21]

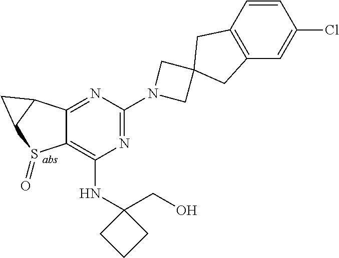

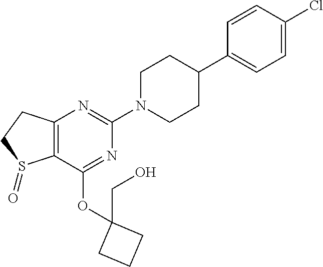 [SMILES: O=[S@]1CCc2nc(N3CCC(c4ccc(Cl)cc4)CC3)nc(OC3(CO)CCC3)c21]

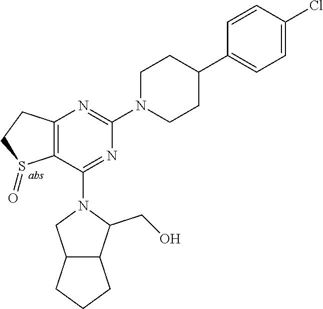 [SMILES: O=[S@]1CCc2nc(N3CCC(c4ccc(Cl)cc4)CC3)nc(N3CC4CCCC4C3CO)c21]

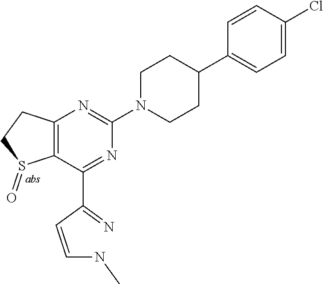 [SMILES: CN/C=C\C(=N)c1nc(N2CCC(c3ccc(Cl)cc3)CC2)nc2c1[S@@](=O)CC2]

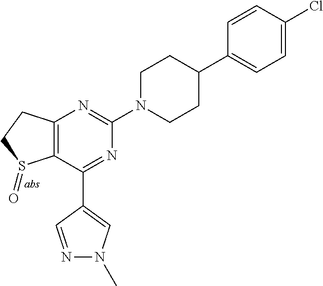 [SMILES: Cn1cc(-c2nc(N3CCC(c4ccc(Cl)cc4)CC3)nc3c2[S@@](=O)CC3)cn1]

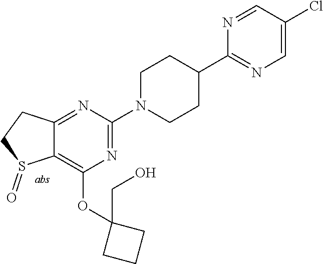 [SMILES: O=[S@]1CCc2nc(N3CCC(c4ncc(Cl)cn4)CC3)nc(OC3(CO)CCC3)c21]

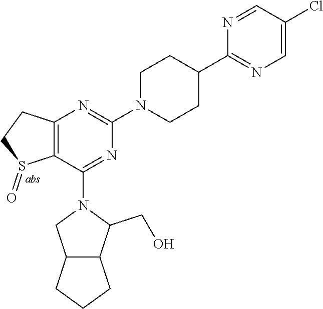 [SMILES: O=[S@]1CCc2nc(N3CCC(c4ncc(Cl)cn4)CC3)nc(N3CC4CCCC4C3CO)c21]

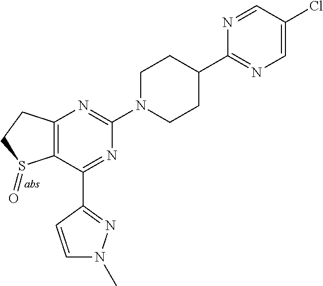 [SMILES: Cn1ccc(-c2nc(N3CCC(c4ncc(Cl)cn4)CC3)nc3c2[S@@](=O)CC3)n1]

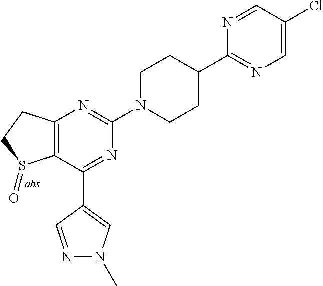 [SMILES: Cn1cc(-c2nc(N3CCC(c4ncc(Cl)cn4)CC3)nc3c2[S@@](=O)CC3)cn1]

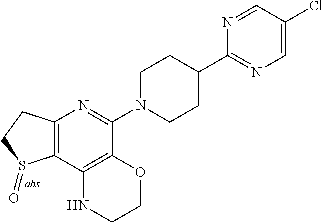 [SMILES: O=[S@]1CCc2nc(N3CCC(c4ncc(Cl)cn4)CC3)c3c(c21)NCCO3]

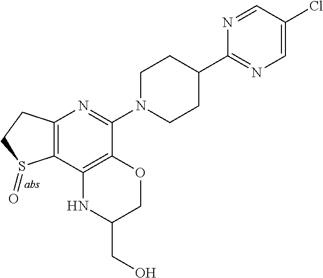 [SMILES: O=[S@]1CCc2nc(N3CCC(c4ncc(Cl)cn4)CC3)c3c(c21)NC(CO)CO3]

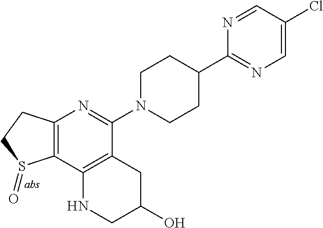 [SMILES: O=[S@]1CCc2nc(N3CCC(c4ncc(Cl)cn4)CC3)c3c(c21)NCC(O)C3]

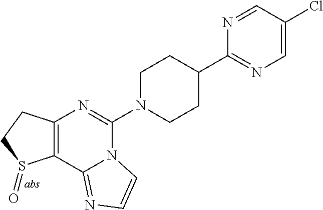 [SMILES: O=[S@]1CCc2nc(N3CCC(c4ncc(Cl)cn4)CC3)n3ccnc3c21]

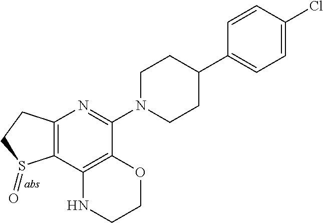 [SMILES: O=[S@]1CCc2nc(N3CCC(c4ccc(Cl)cc4)CC3)c3c(c21)NCCO3]

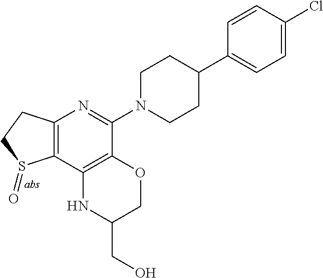 [SMILES: O=[S@]1CCc2nc(N3CCC(c4ccc(Cl)cc4)CC3)c3c(c21)NC(CO)CO3]

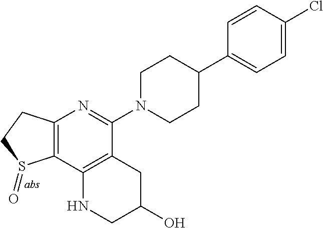 [SMILES: O=[S@]1CCc2nc(N3CCC(c4ccc(Cl)cc4)CC3)c3c(c21)NCC(O)C3]

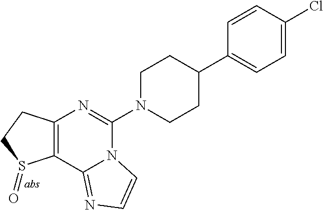 [SMILES: O=[S@]1CCc2nc(N3CCC(c4ccc(Cl)cc4)CC3)n3ccnc3c21]

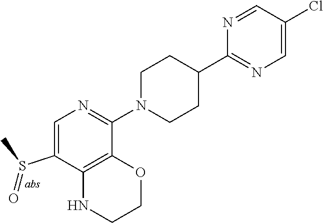 [SMILES: C[S@](=O)c1cnc(N2CCC(c3ncc(Cl)cn3)CC2)c2c1NCCO2]

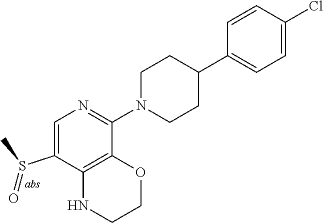 [SMILES: C[S@](=O)c1cnc(N2CCC(c3ccc(Cl)cc3)CC2)c2c1NCCO2]

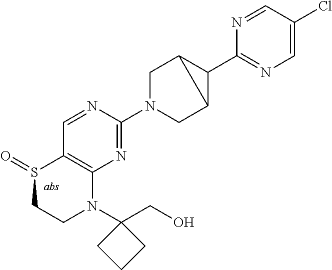 [SMILES: O=[S@@]1CCN(C2(CO)CCC2)c2nc(N3CC4C(C3)C4c3ncc(Cl)cn3)ncc21]

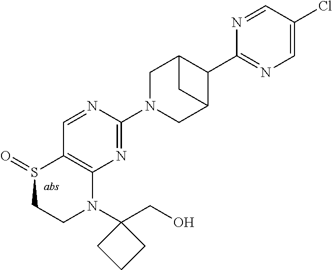 [SMILES: O=[S@@]1CCN(C2(CO)CCC2)c2nc(N3CC4CC(C3)C4c3ncc(Cl)cn3)ncc21]

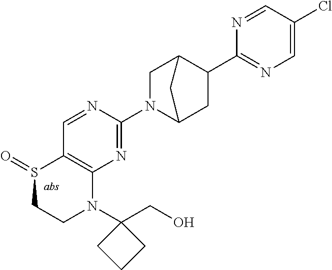 [SMILES: O=[S@@]1CCN(C2(CO)CCC2)c2nc(N3CC4CC3CC4c3ncc(Cl)cn3)ncc21]

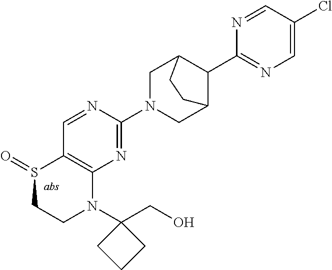 [SMILES: O=[S@@]1CCN(C2(CO)CCC2)c2nc(N3CC4CCC(C3)C4c3ncc(Cl)cn3)ncc21]

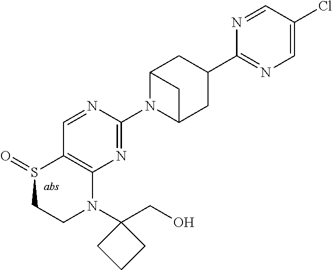 [SMILES: O=[S@@]1CCN(C2(CO)CCC2)c2nc(N3C4CC(c5ncc(Cl)cn5)CC3C4)ncc21]

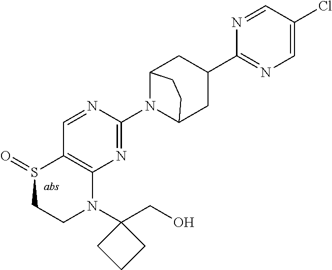 [SMILES: O=[S@@]1CCN(C2(CO)CCC2)c2nc(N3C4CCC3CC(c3ncc(Cl)cn3)C4)ncc21]

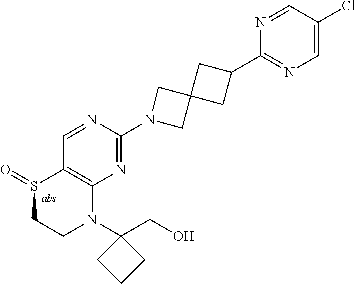 [SMILES: O=[S@@]1CCN(C2(CO)CCC2)c2nc(N3CC4(CC(c5ncc(Cl)cn5)C4)C3)ncc21]

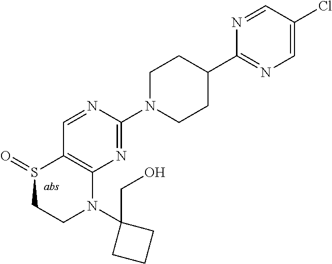 [SMILES: O=[S@@]1CCN(C2(CO)CCC2)c2nc(N3CCC(c4ncc(Cl)cn4)CC3)ncc21]

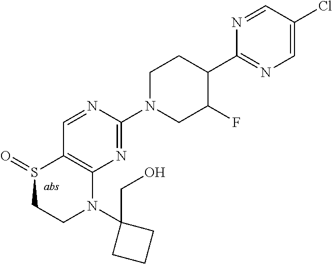 [SMILES: O=[S@@]1CCN(C2(CO)CCC2)c2nc(N3CCC(c4ncc(Cl)cn4)C(F)C3)ncc21]

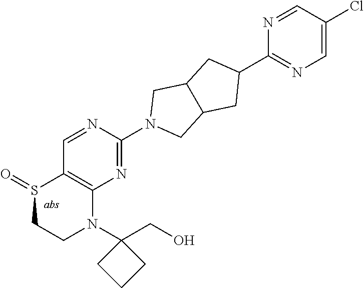 [SMILES: O=[S@@]1CCN(C2(CO)CCC2)c2nc(N3CC4CC(c5ncc(Cl)cn5)CC4C3)ncc21]

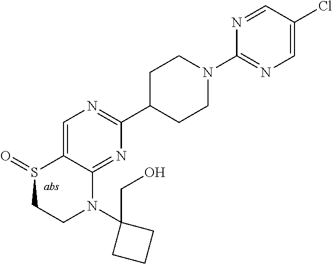 [SMILES: O=[S@@]1CCN(C2(CO)CCC2)c2nc(C3CCN(c4ncc(Cl)cn4)CC3)ncc21]

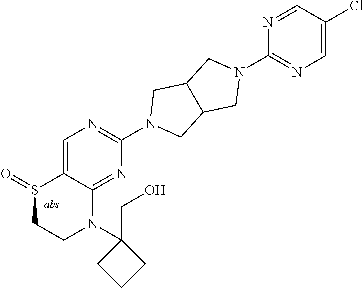 [SMILES: O=[S@@]1CCN(C2(CO)CCC2)c2nc(N3CC4CN(c5ncc(Cl)cn5)CC4C3)ncc21]

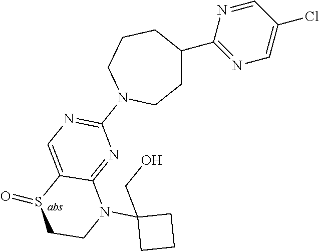 [SMILES: O=[S@@]1CCN(C2(CO)CCC2)c2nc(N3CCCC(c4ncc(Cl)cn4)CC3)ncc21]

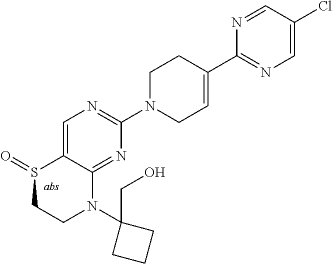 [SMILES: O=[S@@]1CCN(C2(CO)CCC2)c2nc(N3CC=C(c4ncc(Cl)cn4)CC3)ncc21]

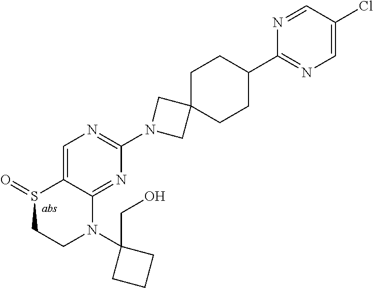 [SMILES: O=[S@@]1CCN(C2(CO)CCC2)c2nc(N3CC4(CCC(c5ncc(Cl)cn5)CC4)C3)ncc21]

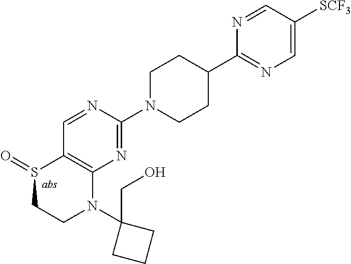

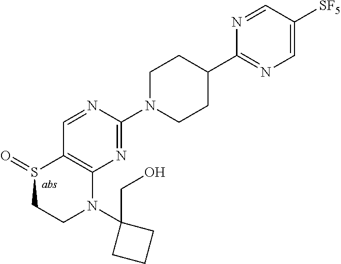

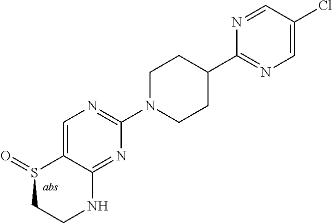 [SMILES: O=[S@@]1CCNc2nc(N3CCC(c4ncc(Cl)cn4)CC3)ncc21]

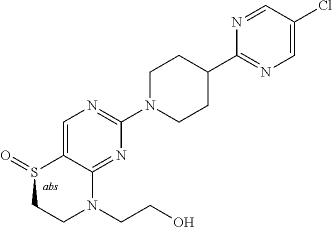 [SMILES: O=[S@@]1CCN(CCO)c2nc(N3CCC(c4ncc(Cl)cn4)CC3)ncc21]

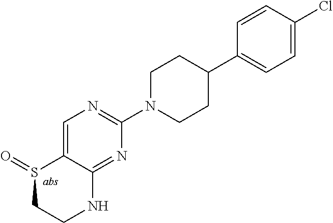 [SMILES: O=[S@@]1CCNc2nc(N3CCC(c4ccc(Cl)cc4)CC3)ncc21]

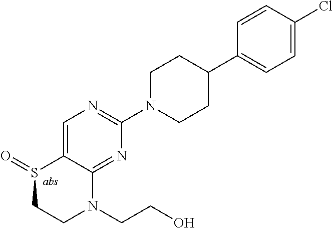 [SMILES: O=[S@@]1CCN(CCO)c2nc(N3CCC(c4ccc(Cl)cc4)CC3)ncc21]

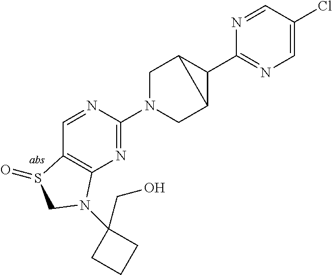 [SMILES: O=[S@@]1CN(C2(CO)CCC2)c2nc(N3CC4C(C3)C4c3ncc(Cl)cn3)ncc21]

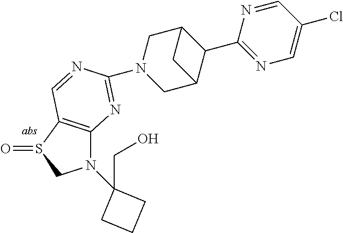 [SMILES: O=[S@@]1CN(C2(CO)CCC2)c2nc(N3CC4CC(C3)C4c3ncc(Cl)cn3)ncc21]

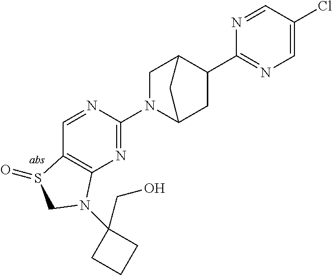 [SMILES: O=[S@@]1CN(C2(CO)CCC2)c2nc(N3CC4CC3CC4c3ncc(Cl)cn3)ncc21]

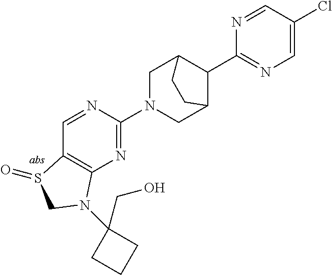 [SMILES: O=[S@@]1CN(C2(CO)CCC2)c2nc(N3CC4CCC(C3)C4c3ncc(Cl)cn3)ncc21]

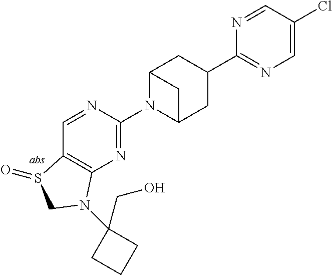 [SMILES: O=[S@@]1CN(C2(CO)CCC2)c2nc(N3C4CC(c5ncc(Cl)cn5)CC3C4)ncc21]

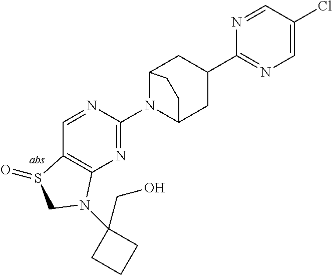 [SMILES: O=[S@@]1CN(C2(CO)CCC2)c2nc(N3C4CCC3CC(c3ncc(Cl)cn3)C4)ncc21]

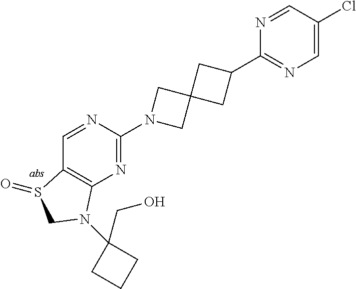 [SMILES: O=[S@@]1CN(C2(CO)CCC2)c2nc(N3CC4(CC(c5ncc(Cl)cn5)C4)C3)ncc21]

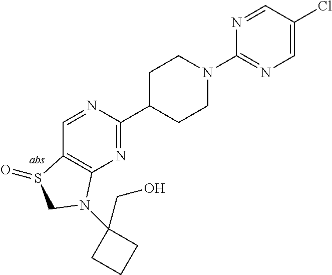 [SMILES: O=[S@@]1CN(C2(CO)CCC2)c2nc(C3CCN(c4ncc(Cl)cn4)CC3)ncc21]

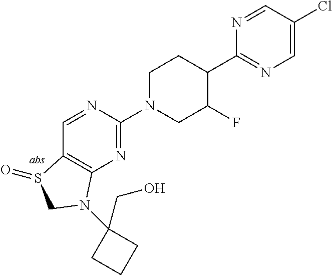 [SMILES: O=[S@@]1CN(C2(CO)CCC2)c2nc(N3CCC(c4ncc(Cl)cn4)C(F)C3)ncc21]

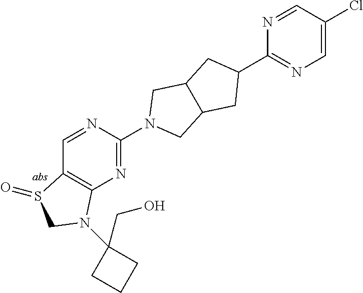 [SMILES: O=[S@@]1CN(C2(CO)CCC2)c2nc(N3CC4CC(c5ncc(Cl)cn5)CC4C3)ncc21]

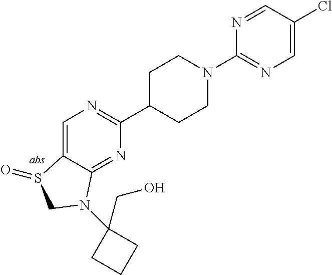 [SMILES: O=[S@@]1CN(C2(CO)CCC2)c2nc(C3CCN(c4ncc(Cl)cn4)CC3)ncc21]

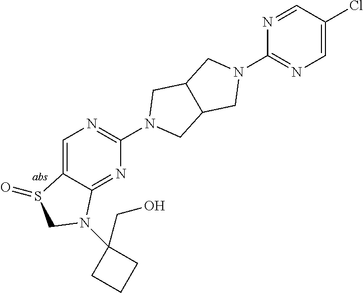 [SMILES: O=[S@@]1CN(C2(CO)CCC2)c2nc(N3CC4CN(c5ncc(Cl)cn5)CC4C3)ncc21]

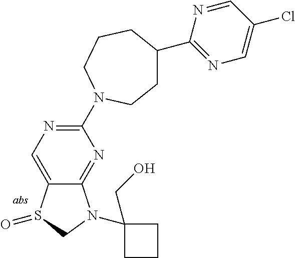 [SMILES: O=[S@@]1CN(C2(CO)CCC2)c2nc(N3CCCC(c4ncc(Cl)cn4)CC3)ncc21]

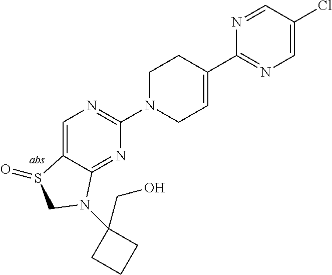 [SMILES: O=[S@@]1CN(C2(CO)CCC2)c2nc(N3CC=C(c4ncc(Cl)cn4)CC3)ncc21]

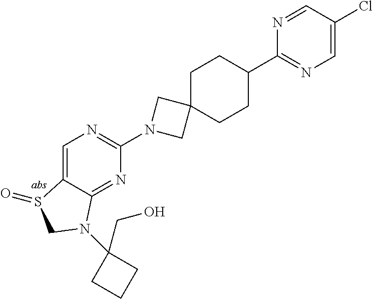 [SMILES: O=[S@@]1CN(C2(CO)CCC2)c2nc(N3CC4(CCC(c5ncc(Cl)cn5)CC4)C3)ncc21]

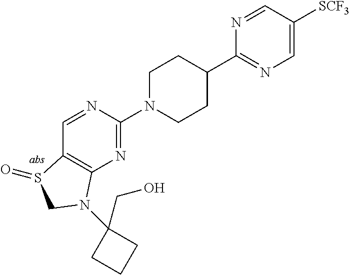

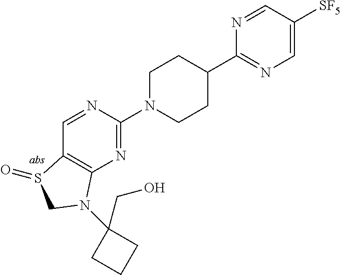

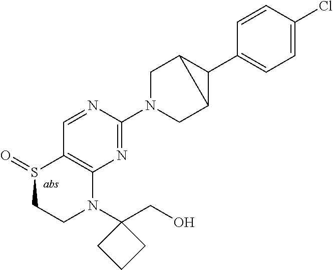 [SMILES: O=[S@@]1CCN(C2(CO)CCC2)c2nc(N3CC4C(C3)C4c3ccc(Cl)cc3)ncc21]

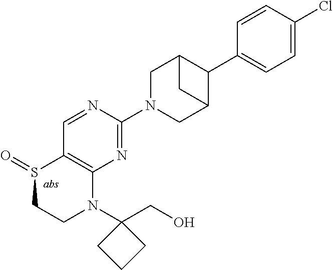 [SMILES: O=[S@@]1CCN(C2(CO)CCC2)c2nc(N3CC4CC(C3)C4c3ccc(Cl)cc3)ncc21]

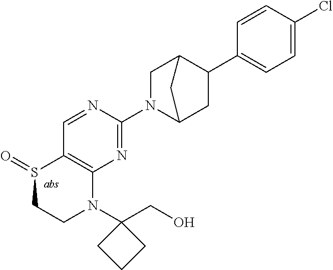 [SMILES: O=[S@@]1CCN(C2(CO)CCC2)c2nc(N3CC4CC3CC4c3ccc(Cl)cc3)ncc21]

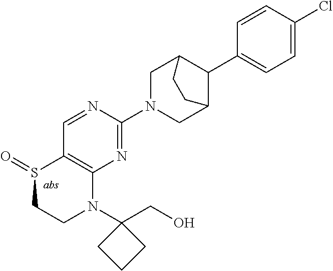 [SMILES: O=[S@@]1CCN(C2(CO)CCC2)c2nc(N3CC4CCC(C3)C4c3ccc(Cl)cc3)ncc21]

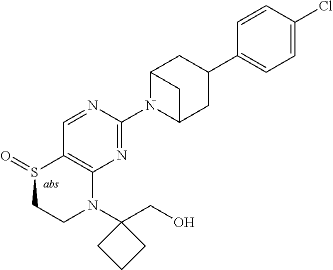 [SMILES: O=[S@@]1CCN(C2(CO)CCC2)c2nc(N3C4CC(c5ccc(Cl)cc5)CC3C4)ncc21]

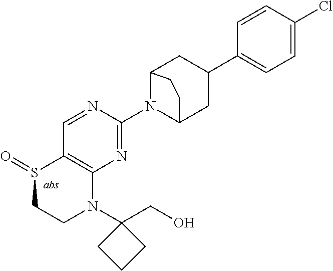 [SMILES: O=[S@@]1CCN(C2(CO)CCC2)c2nc(N3C4CCC3CC(c3ccc(Cl)cc3)C4)ncc21]

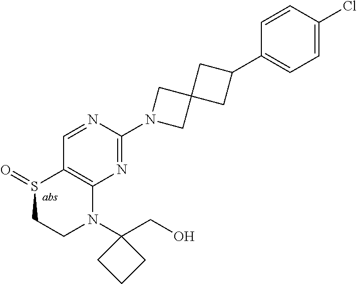 [SMILES: O=[S@@]1CCN(C2(CO)CCC2)c2nc(N3CC4(CC(c5ccc(Cl)cc5)C4)C3)ncc21]

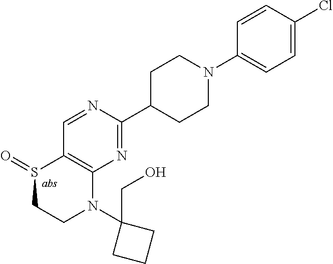 [SMILES: O=[S@@]1CCN(C2(CO)CCC2)c2nc(C3CCN(c4ccc(Cl)cc4)CC3)ncc21]

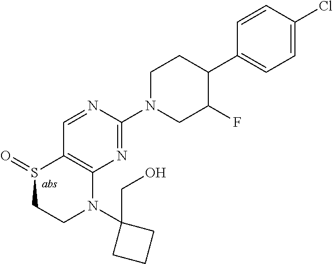 [SMILES: O=[S@@]1CCN(C2(CO)CCC2)c2nc(N3CCC(c4ccc(Cl)cc4)C(F)C3)ncc21]

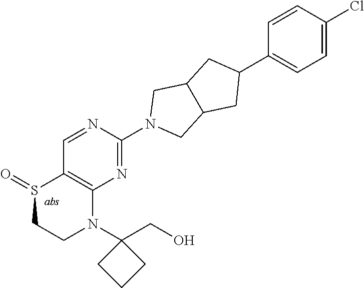 [SMILES: O=[S@@]1CCN(C2(CO)CCC2)c2nc(N3CC4CC(c5ccc(Cl)cc5)CC4C3)ncc21]

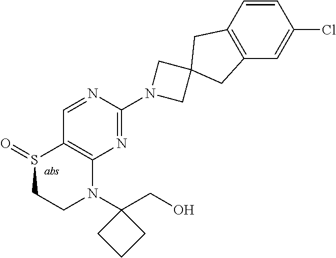 [SMILES: O=[S@@]1CCN(C2(CO)CCC2)c2nc(N3CC4(Cc5ccc(Cl)cc5C4)C3)ncc21]

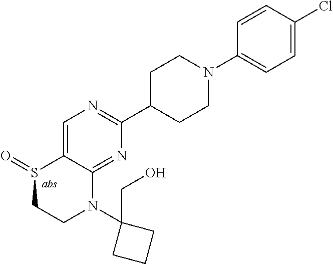 [SMILES: O=[S@@]1CCN(C2(CO)CCC2)c2nc(C3CCN(c4ccc(Cl)cc4)CC3)ncc21]

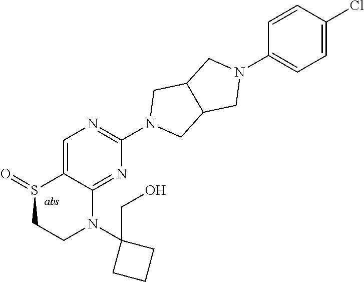 [SMILES: O=[S@@]1CCN(C2(CO)CCC2)c2nc(N3CC4CN(c5ccc(Cl)cc5)CC4C3)ncc21]

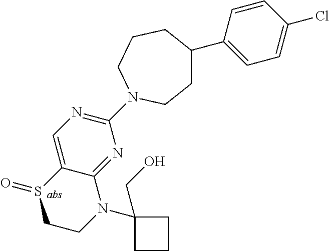 [SMILES: O=[S@@]1CCN(C2(CO)CCC2)c2nc(N3CCCC(c4ccc(Cl)cc4)CC3)ncc21]

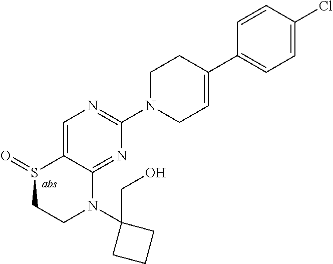 [SMILES: O=[S@@]1CCN(C2(CO)CCC2)c2nc(N3CC=C(c4ccc(Cl)cc4)CC3)ncc21]

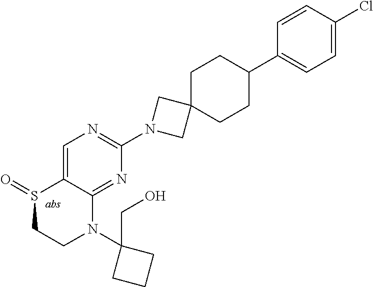 [SMILES: O=[S@@]1CCN(C2(CO)CCC2)c2nc(N3CC4(CCC(c5ccc(Cl)cc5)CC4)C3)ncc21]

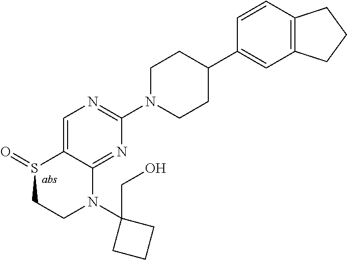 [SMILES: O=[S@@]1CCN(C2(CO)CCC2)c2nc(N3CCC(c4ccc5c(c4)CCC5)CC3)ncc21]

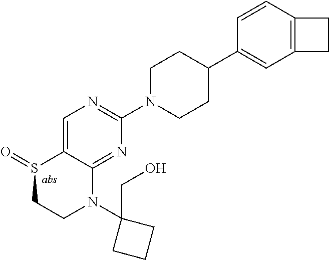 [SMILES: O=[S@@]1CCN(C2(CO)CCC2)c2nc(N3CCC(c4ccc5c(c4)CC5)CC3)ncc21]

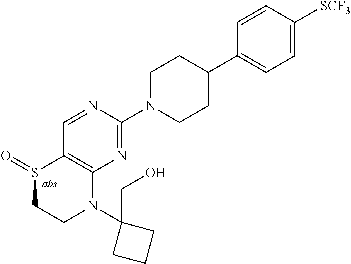

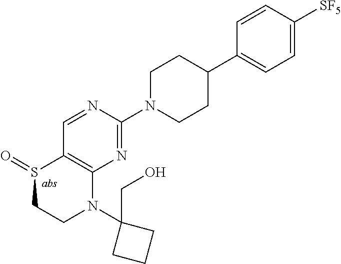

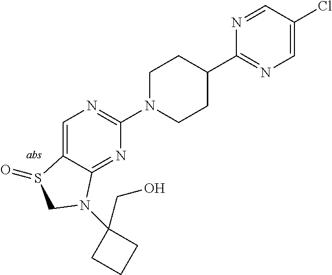 [SMILES: O=[S@@]1CN(C2(CO)CCC2)c2nc(N3CCC(c4ncc(Cl)cn4)CC3)ncc21]

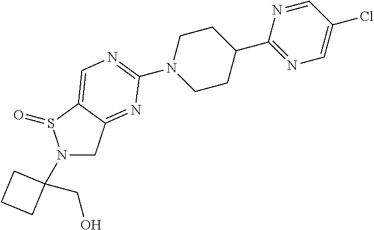 [SMILES: O=S1c2cnc(N3CCC(c4ncc(Cl)cn4)CC3)nc2CN1C1(CO)CCC1]

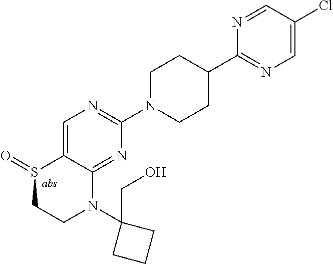 [SMILES: O=[S@@]1CCN(C2(CO)CCC2)c2nc(N3CCC(c4ncc(Cl)cn4)CC3)ncc21]

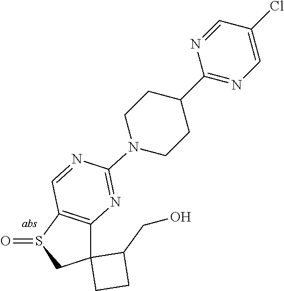

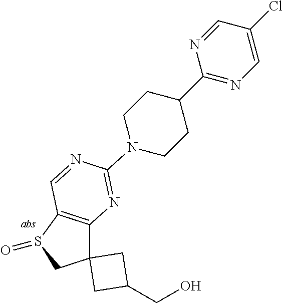

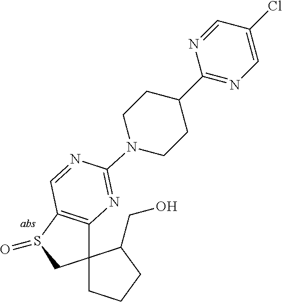

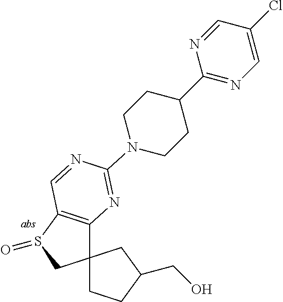

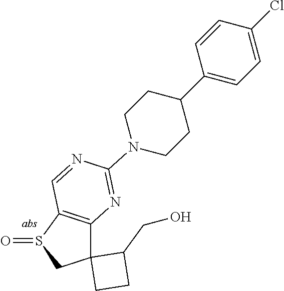

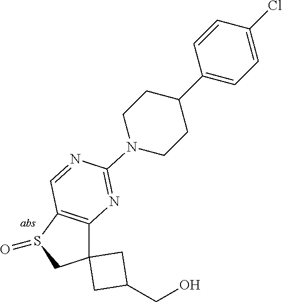

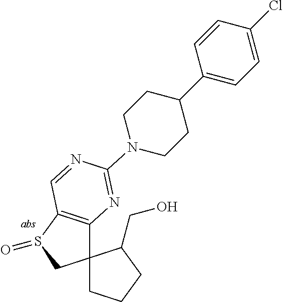

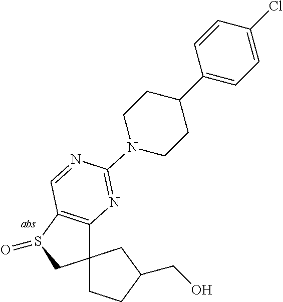

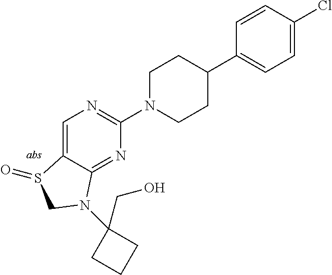 [SMILES: O=[S@@]1CN(C2(CO)CCC2)c2nc(N3CCC(c4ccc(Cl)cc4)CC3)ncc21]

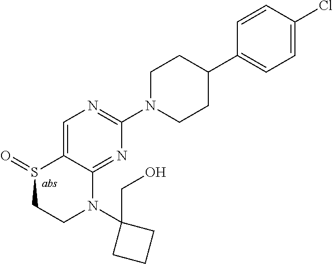 [SMILES: O=[S@@]1CCN(C2(CO)CCC2)c2nc(N3CCC(c4ccc(Cl)cc4)CC3)ncc21]

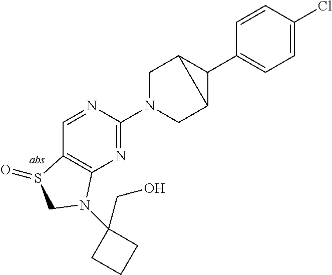 [SMILES: O=[S@@]1CN(C2(CO)CCC2)c2nc(N3CC4C(C3)C4c3ccc(Cl)cc3)ncc21]

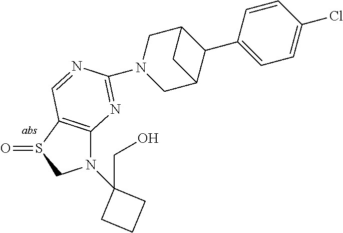 [SMILES: O=[S@@]1CN(C2(CO)CCC2)c2nc(N3CC4CC(C3)C4c3ccc(Cl)cc3)ncc21]

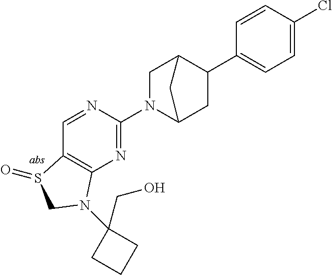 [SMILES: O=[S@@]1CN(C2(CO)CCC2)c2nc(N3CC4CC3CC4c3ccc(Cl)cc3)ncc21]

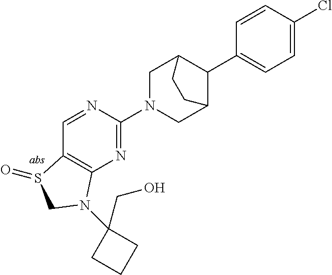 [SMILES: O=[S@@]1CN(C2(CO)CCC2)c2nc(N3CC4CCC(C3)C4c3ccc(Cl)cc3)ncc21]

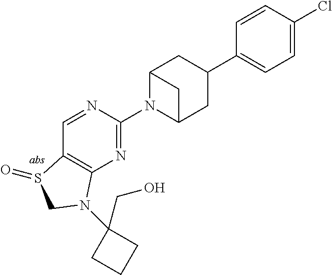 [SMILES: O=[S@@]1CN(C2(CO)CCC2)c2nc(N3C4CC(c5ccc(Cl)cc5)CC3C4)ncc21]

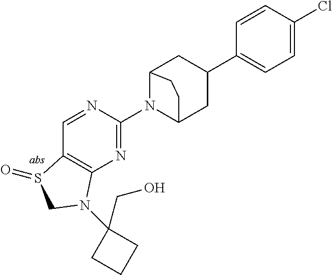 [SMILES: O=[S@@]1CN(C2(CO)CCC2)c2nc(N3C4CCC3CC(c3ccc(Cl)cc3)C4)ncc21]

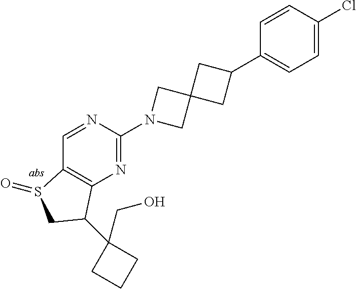

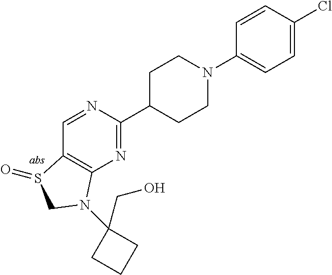 [SMILES: O=[S@@]1CN(C2(CO)CCC2)c2nc(C3CCN(c4ccc(Cl)cc4)CC3)ncc21]

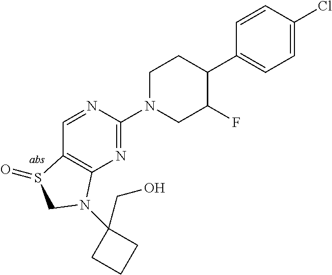 [SMILES: O=[S@@]1CN(C2(CO)CCC2)c2nc(N3CCC(c4ccc(Cl)cc4)C(F)C3)ncc21]

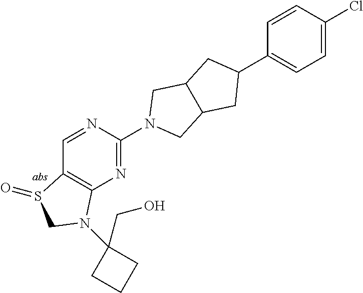 [SMILES: O=[S@@]1CN(C2(CO)CCC2)c2nc(N3CC4CC(c5ccc(Cl)cc5)CC4C3)ncc21]

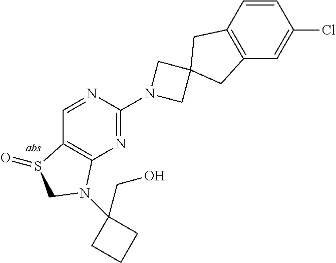 [SMILES: O=[S@@]1CN(C2(CO)CCC2)c2nc(N3CC4(Cc5ccc(Cl)cc5C4)C3)ncc21]

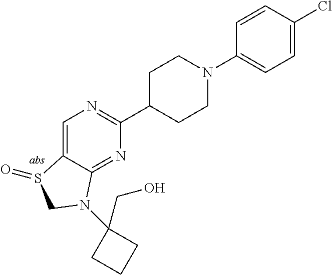 [SMILES: O=[S@@]1CN(C2(CO)CCC2)c2nc(C3CCN(c4ccc(Cl)cc4)CC3)ncc21]

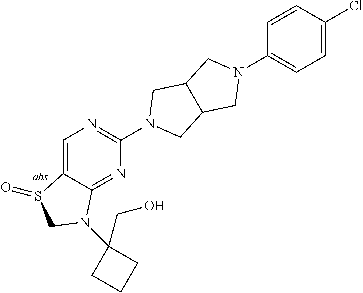 [SMILES: O=[S@@]1CN(C2(CO)CCC2)c2nc(N3CC4CN(c5ccc(Cl)cc5)CC4C3)ncc21]

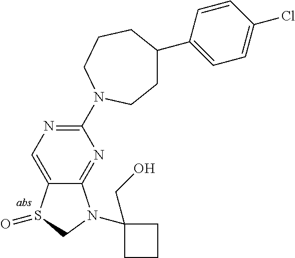 [SMILES: O=[S@@]1CN(C2(CO)CCC2)c2nc(N3CCCC(c4ccc(Cl)cc4)CC3)ncc21]

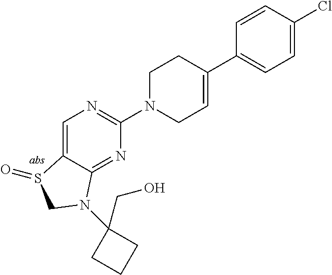 [SMILES: O=[S@@]1CN(C2(CO)CCC2)c2nc(N3CC=C(c4ccc(Cl)cc4)CC3)ncc21]

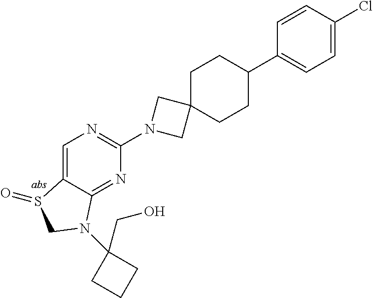 [SMILES: O=[S@@]1CN(C2(CO)CCC2)c2nc(N3CC4(CCC(c5ccc(Cl)cc5)CC4)C3)ncc21]

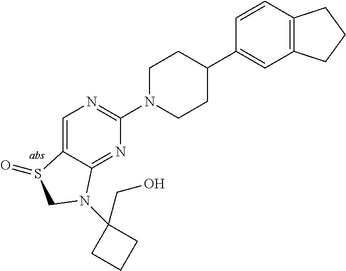 [SMILES: O=[S@@]1CN(C2(CO)CCC2)c2nc(N3CCC(c4ccc5c(c4)CCC5)CC3)ncc21]

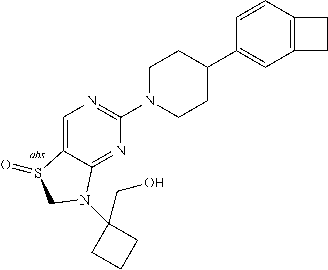 [SMILES: O=[S@@]1CN(C2(CO)CCC2)c2nc(N3CCC(c4ccc5c(c4)CC5)CC3)ncc21]

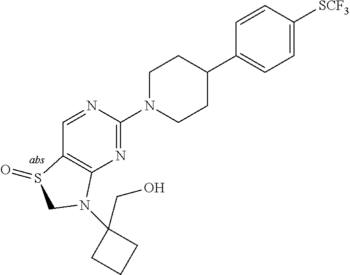

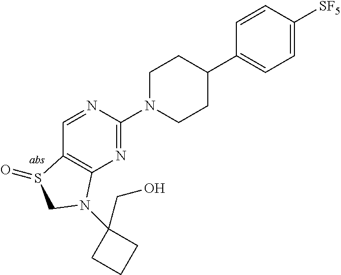

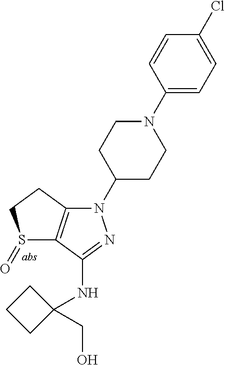 [SMILES: O=[S@]1CCc2c1c(NC1(CO)CCC1)nn2C1CCN(c2ccc(Cl)cc2)CC1]

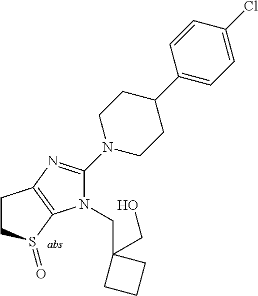 [SMILES: O=[S@]1CCc2nc(N3CCC(c4ccc(Cl)cc4)CC3)n(CC3(CO)CCC3)c21]

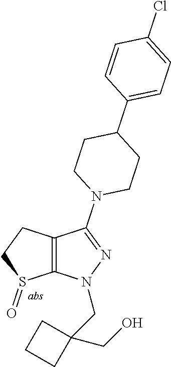 [SMILES: O=[S@]1CCc2c(N3CCC(c4ccc(Cl)cc4)CC3)nn(CC3(CO)CCC3)c21]

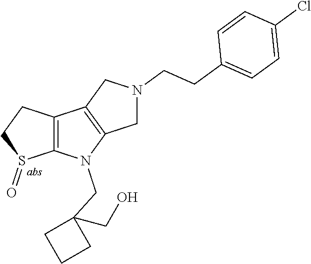 [SMILES: O=[S@]1CCc2c3c(n(CC4(CO)CCC4)c21)CN(CCc1ccc(Cl)cc1)C3]

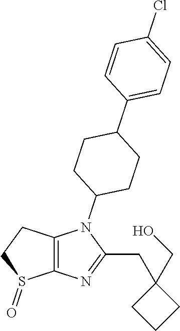 [SMILES: O=[S@]1CCc2c1nc(CC1(CO)CCC1)n2C1CCC(c2ccc(Cl)cc2)CC1]

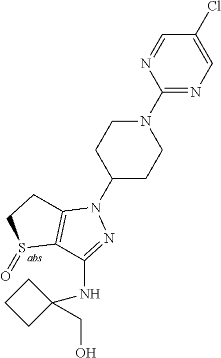 [SMILES: O=[S@]1CCc2c1c(NC1(CO)CCC1)nn2C1CCN(c2ncc(Cl)cn2)CC1]

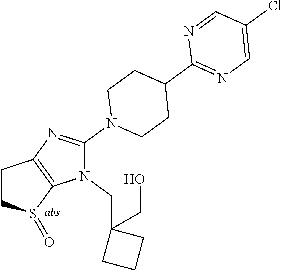 [SMILES: O=[S@]1CCc2nc(N3CCC(c4ncc(Cl)cn4)CC3)n(CC3(CO)CCC3)c21]

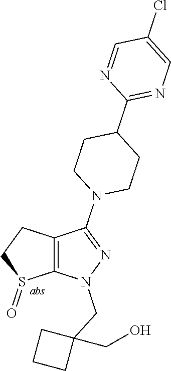 [SMILES: O=[S@]1CCc2c(N3CCC(c4ncc(Cl)cn4)CC3)nn(CC3(CO)CCC3)c21]

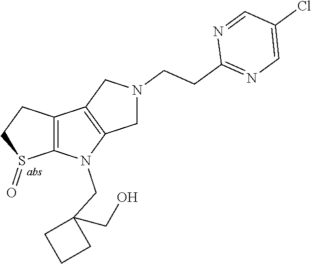 [SMILES: O=[S@]1CCc2c3c(n(CC4(CO)CCC4)c21)CN(CCc1ncc(Cl)cn1)C3]

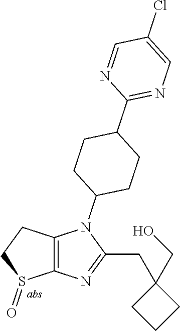 [SMILES: O=[S@]1CCc2c1nc(CC1(CO)CCC1)n2C1CCC(c2ncc(Cl)cn2)CC1]

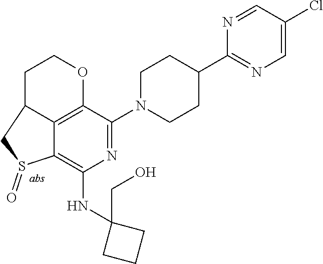

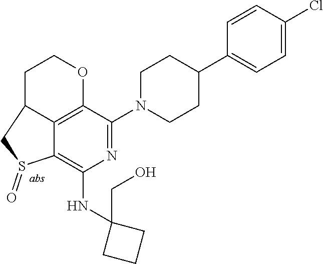

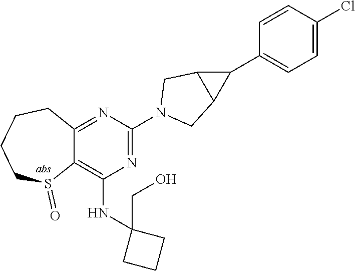 [SMILES: O=[S@]1CCCCc2nc(N3CC4C(C3)C4c3ccc(Cl)cc3)nc(NC3(CO)CCC3)c21]

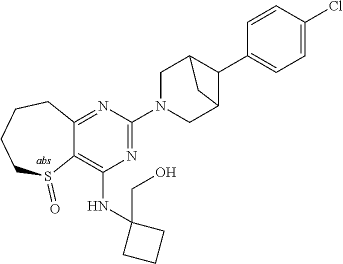 [SMILES: O=[S@]1CCCCc2nc(N3CC4CC(C3)C4c3ccc(Cl)cc3)nc(NC3(CO)CCC3)c21]

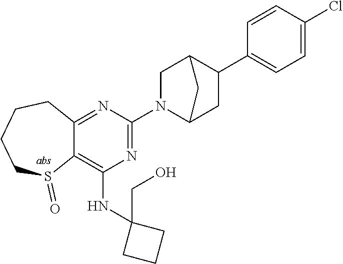 [SMILES: O=[S@]1CCCCc2nc(N3CC4CC3CC4c3ccc(Cl)cc3)nc(NC3(CO)CCC3)c21]

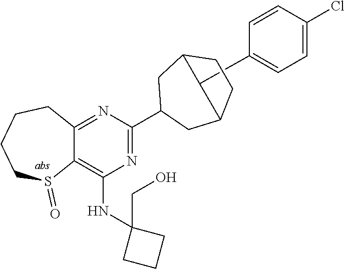 [SMILES: O=[S@]1CCCCc2nc(C3CC4CCC(C3)C4c3ccc(Cl)cc3)nc(NC3(CO)CCC3)c21]

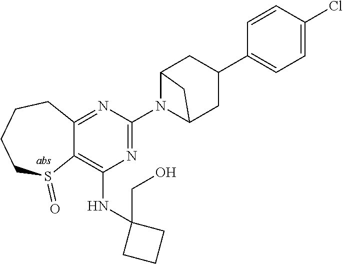 [SMILES: O=[S@]1CCCCc2nc(N3C4CC(c5ccc(Cl)cc5)CC3C4)nc(NC3(CO)CCC3)c21]

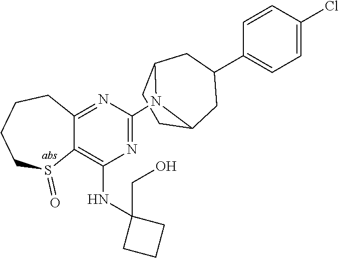 [SMILES: O=[S@]1CCCCc2nc(N3C4CCC3CC(c3ccc(Cl)cc3)C4)nc(NC3(CO)CCC3)c21]

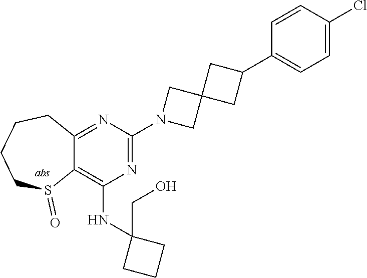 [SMILES: O=[S@]1CCCCc2nc(N3CC4(CC(c5ccc(Cl)cc5)C4)C3)nc(NC3(CO)CCC3)c21]

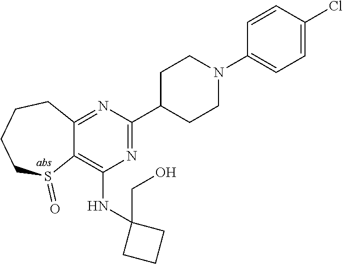 [SMILES: O=[S@]1CCCCc2nc(C3CCN(c4ccc(Cl)cc4)CC3)nc(NC3(CO)CCC3)c21]

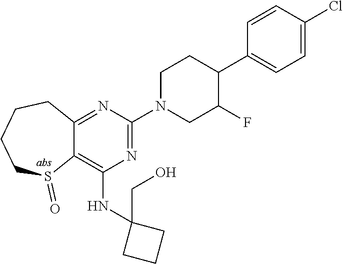 [SMILES: O=[S@]1CCCCc2nc(N3CCC(c4ccc(Cl)cc4)C(F)C3)nc(NC3(CO)CCC3)c21]

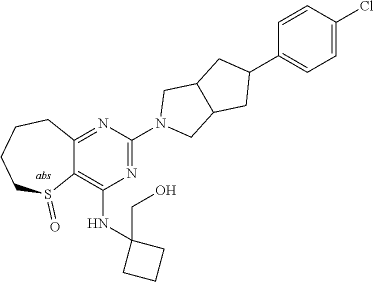 [SMILES: O=[S@]1CCCCc2nc(N3CC4CC(c5ccc(Cl)cc5)CC4C3)nc(NC3(CO)CCC3)c21]

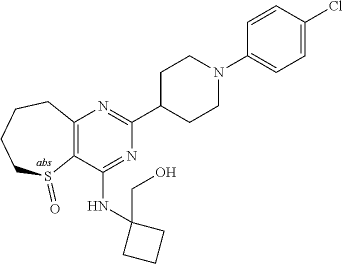 [SMILES: O=[S@]1CCCCc2nc(C3CCN(c4ccc(Cl)cc4)CC3)nc(NC3(CO)CCC3)c21]

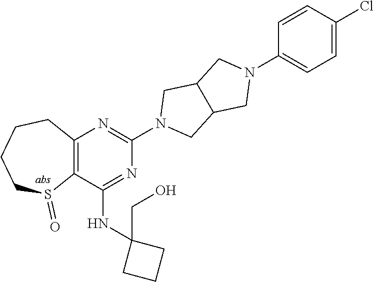 [SMILES: O=[S@]1CCCCc2nc(N3CC4CN(c5ccc(Cl)cc5)CC4C3)nc(NC3(CO)CCC3)c21]

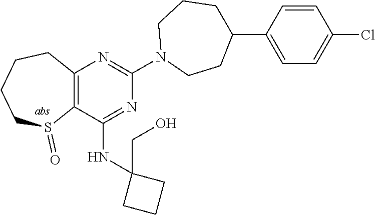 [SMILES: O=[S@]1CCCCc2nc(N3CCCC(c4ccc(Cl)cc4)CC3)nc(NC3(CO)CCC3)c21]

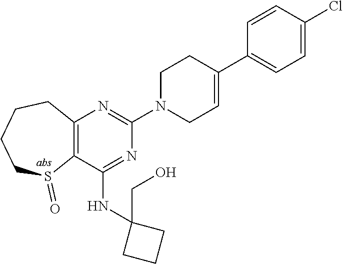 [SMILES: O=[S@]1CCCCc2nc(N3CC=C(c4ccc(Cl)cc4)CC3)nc(NC3(CO)CCC3)c21]

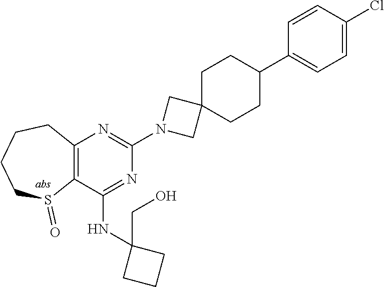 [SMILES: O=[S@]1CCCCc2nc(N3CC4(CCC(c5ccc(Cl)cc5)CC4)C3)nc(NC3(CO)CCC3)c21]

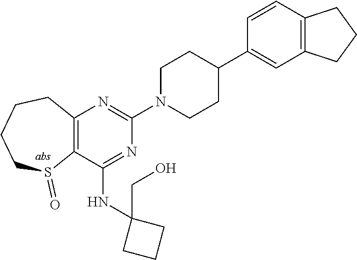 [SMILES: O=[S@]1CCCCc2nc(N3CCC(c4ccc5c(c4)CCC5)CC3)nc(NC3(CO)CCC3)c21]

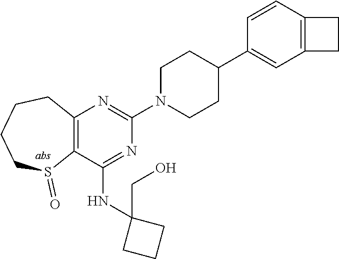 [SMILES: O=[S@]1CCCCc2nc(N3CCC(c4ccc5c(c4)CC5)CC3)nc(NC3(CO)CCC3)c21]

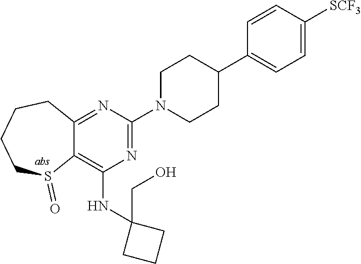

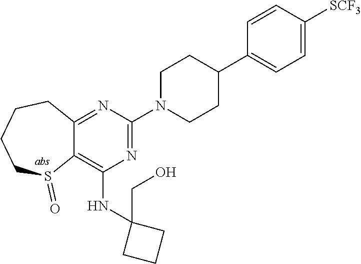

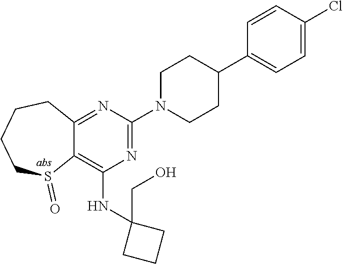 [SMILES: O=[S@]1CCCCc2nc(N3CCC(c4ccc(Cl)cc4)CC3)nc(NC3(CO)CCC3)c21]

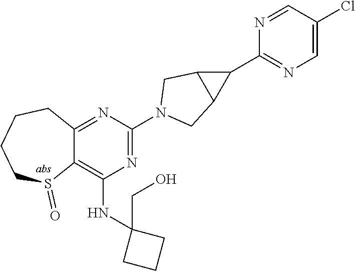 [SMILES: O=[S@]1CCCCc2nc(N3CC4C(C3)C4c3ncc(Cl)cn3)nc(NC3(CO)CCC3)c21]

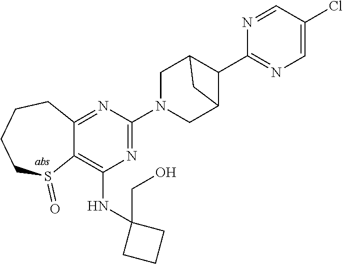 [SMILES: O=[S@]1CCCCc2nc(N3CC4CC(C3)C4c3ncc(Cl)cn3)nc(NC3(CO)CCC3)c21]

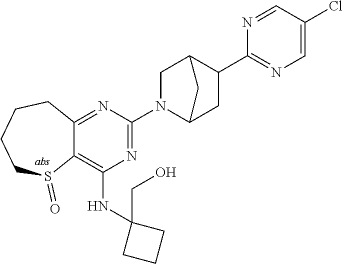 [SMILES: O=[S@]1CCCCc2nc(N3CC4CC3CC4c3ncc(Cl)cn3)nc(NC3(CO)CCC3)c21]

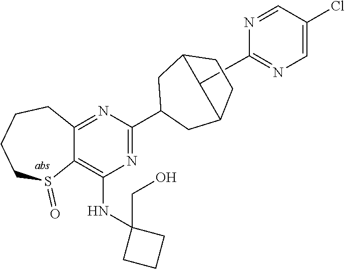 [SMILES: O=[S@]1CCCCc2nc(C3CC4CCC(C3)C4c3ncc(Cl)cn3)nc(NC3(CO)CCC3)c21]

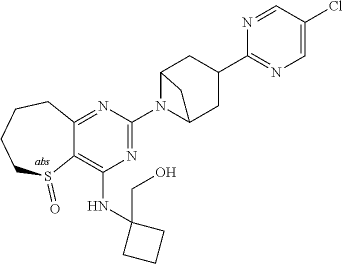 [SMILES: O=[S@]1CCCCc2nc(N3C4CC(c5ncc(Cl)cn5)CC3C4)nc(NC3(CO)CCC3)c21]

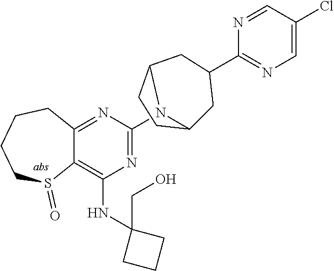 [SMILES: O=[S@]1CCCCc2nc(N3C4CCC3CC(c3ncc(Cl)cn3)C4)nc(NC3(CO)CCC3)c21]

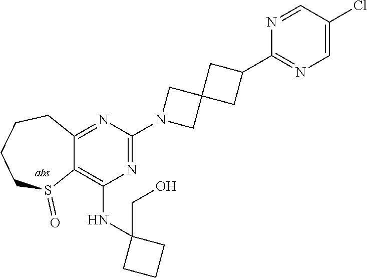 [SMILES: O=[S@]1CCCCc2nc(N3CC4(CC(c5ncc(Cl)cn5)C4)C3)nc(NC3(CO)CCC3)c21]

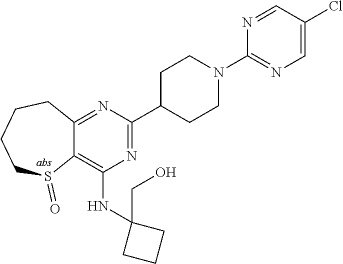 [SMILES: O=[S@]1CCCCc2nc(C3CCN(c4ncc(Cl)cn4)CC3)nc(NC3(CO)CCC3)c21]

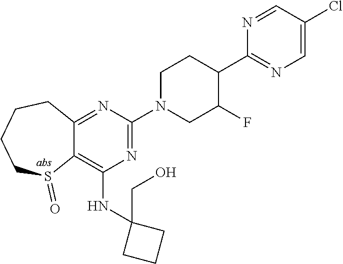 [SMILES: O=[S@]1CCCCc2nc(N3CCC(c4ncc(Cl)cn4)C(F)C3)nc(NC3(CO)CCC3)c21]

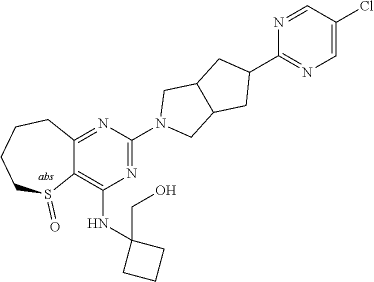 [SMILES: O=[S@]1CCCCc2nc(N3CC4CC(c5ncc(Cl)cn5)CC4C3)nc(NC3(CO)CCC3)c21]

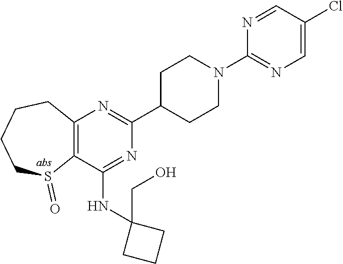 [SMILES: O=[S@]1CCCCc2nc(C3CCN(c4ncc(Cl)cn4)CC3)nc(NC3(CO)CCC3)c21]

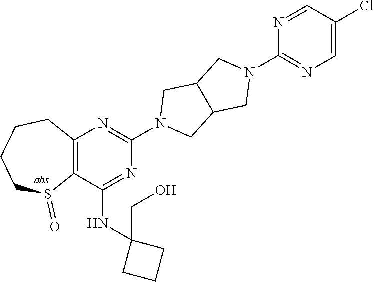 [SMILES: O=[S@]1CCCCc2nc(N3CC4CN(c5ncc(Cl)cn5)CC4C3)nc(NC3(CO)CCC3)c21]

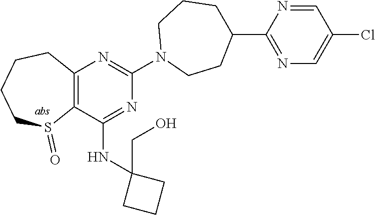 [SMILES: O=[S@]1CCCCc2nc(N3CCCC(c4ncc(Cl)cn4)CC3)nc(NC3(CO)CCC3)c21]

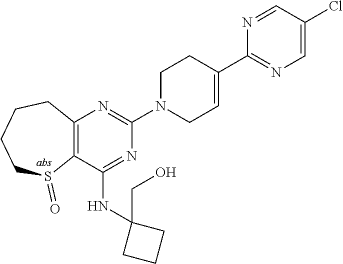 [SMILES: O=[S@]1CCCCc2nc(N3CC=C(c4ncc(Cl)cn4)CC3)nc(NC3(CO)CCC3)c21]

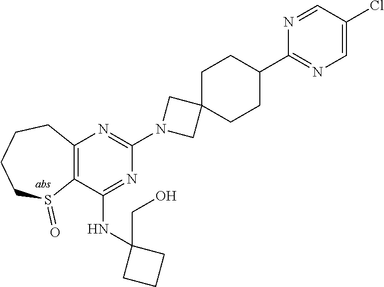 [SMILES: O=[S@]1CCCCc2nc(N3CC4(CCC(c5ncc(Cl)cn5)CC4)C3)nc(NC3(CO)CCC3)c21]

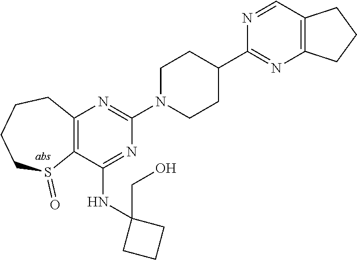 [SMILES: O=[S@]1CCCCc2nc(N3CCC(c4ncc5c(n4)CCC5)CC3)nc(NC3(CO)CCC3)c21]

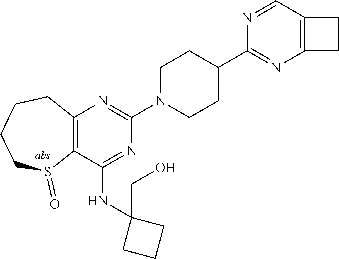 [SMILES: O=[S@]1CCCCc2nc(N3CCC(c4ncc5c(n4)CC5)CC3)nc(NC3(CO)CCC3)c21]

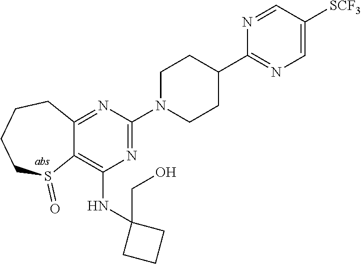

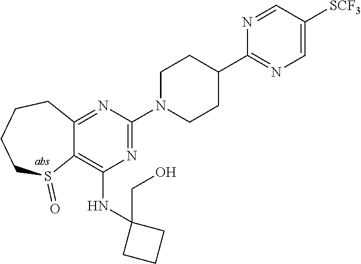

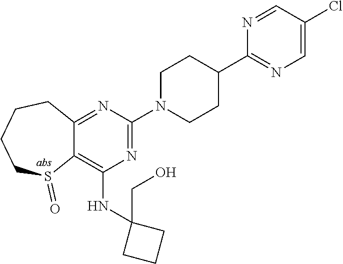 [SMILES: O=[S@]1CCCCc2nc(N3CCC(c4ncc(Cl)cn4)CC3)nc(NC3(CO)CCC3)c21]

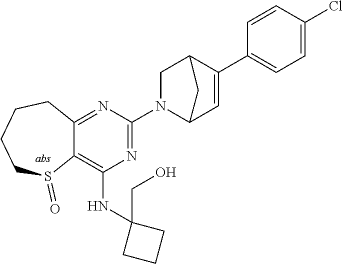 [SMILES: O=[S@]1CCCCc2nc(N3CC4CC3C=C4c3ccc(Cl)cc3)nc(NC3(CO)CCC3)c21]

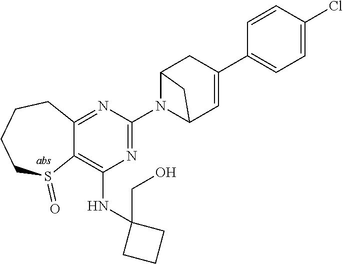 [SMILES: O=[S@]1CCCCc2nc(N3C4C=C(c5ccc(Cl)cc5)CC3C4)nc(NC3(CO)CCC3)c21]

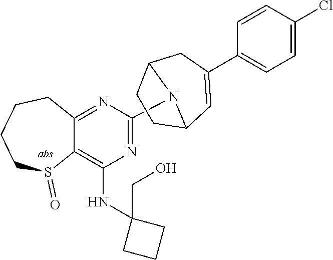 [SMILES: O=[S@]1CCCCc2nc(N3C4C=C(c5ccc(Cl)cc5)CC3CC4)nc(NC3(CO)CCC3)c21]

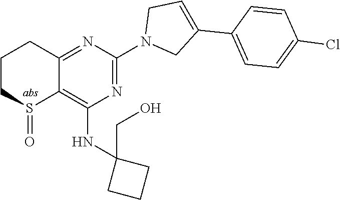 [SMILES: O=[S@]1CCCc2nc(N3CC=C(c4ccc(Cl)cc4)C3)nc(NC3(CO)CCC3)c21]

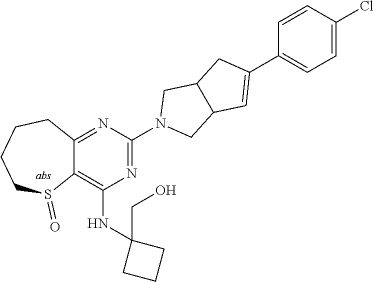 [SMILES: O=[S@]1CCCCc2nc(N3CC4C=C(c5ccc(Cl)cc5)CC4C3)nc(NC3(CO)CCC3)c21]

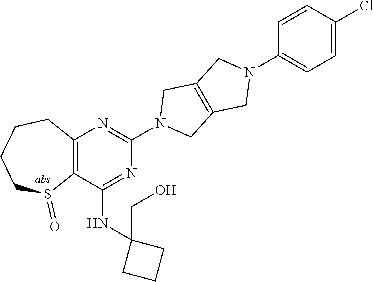 [SMILES: O=[S@]1CCCCc2nc(N3CC4=C(CN(c5ccc(Cl)cc5)C4)C3)nc(NC3(CO)CCC3)c21]

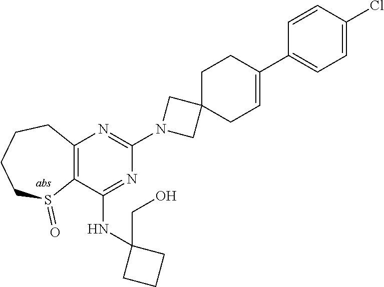 [SMILES: O=[S@]1CCCCc2nc(N3CC4(CC=C(c5ccc(Cl)cc5)CC4)C3)nc(NC3(CO)CCC3)c21]

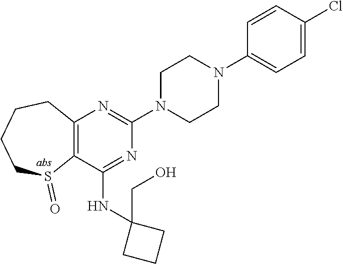 [SMILES: O=[S@]1CCCCc2nc(N3CCN(c4ccc(Cl)cc4)CC3)nc(NC3(CO)CCC3)c21]

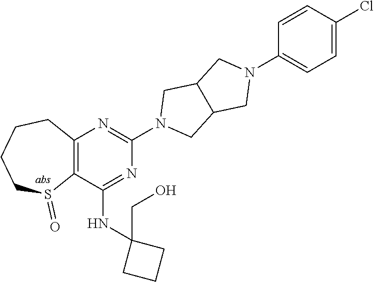 [SMILES: O=[S@]1CCCCc2nc(N3CC4CN(c5ccc(Cl)cc5)CC4C3)nc(NC3(CO)CCC3)c21]

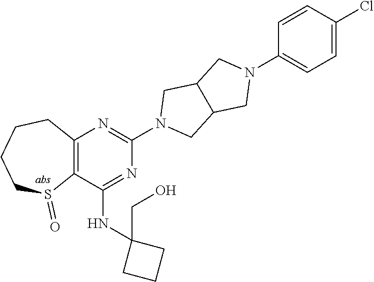 [SMILES: O=[S@]1CCCCc2nc(N3CC4CN(c5ccc(Cl)cc5)CC4C3)nc(NC3(CO)CCC3)c21]

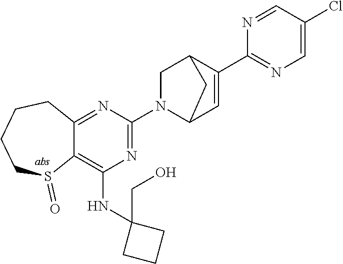 [SMILES: O=[S@]1CCCCc2nc(N3CC4CC3C=C4c3ncc(Cl)cn3)nc(NC3(CO)CCC3)c21]

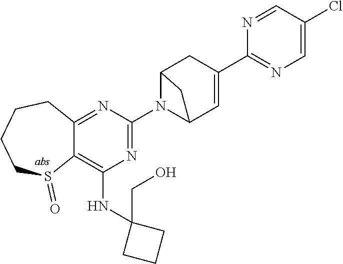 [SMILES: O=[S@]1CCCCc2nc(N3C4C=C(c5ncc(Cl)cn5)CC3C4)nc(NC3(CO)CCC3)c21]

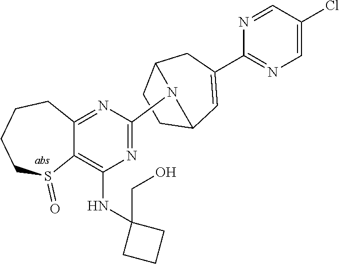 [SMILES: O=[S@]1CCCCc2nc(N3C4C=C(c5ncc(Cl)cn5)CC3CC4)nc(NC3(CO)CCC3)c21]

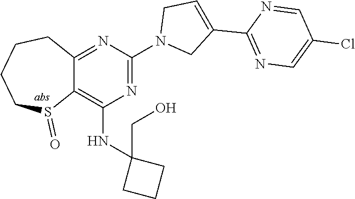

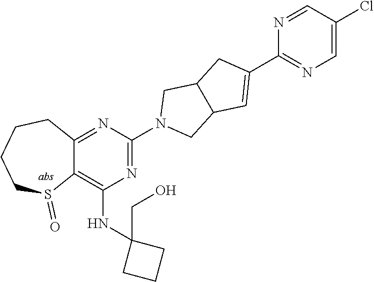 [SMILES: O=[S@]1CCCCc2nc(N3CC4C=C(c5ncc(Cl)cn5)CC4C3)nc(NC3(CO)CCC3)c21]

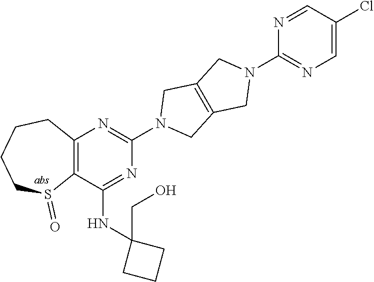 [SMILES: O=[S@]1CCCCc2nc(N3CC4=C(CN(c5ncc(Cl)cn5)C4)C3)nc(NC3(CO)CCC3)c21]

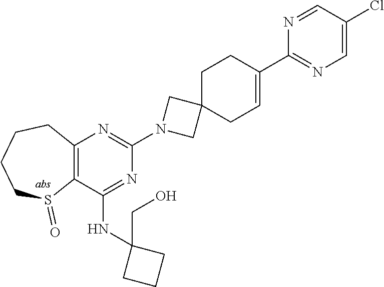 [SMILES: O=[S@]1CCCCc2nc(N3CC4(CC=C(c5ncc(Cl)cn5)CC4)C3)nc(NC3(CO)CCC3)c21]

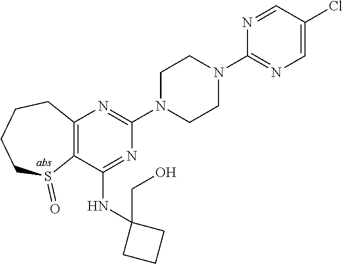 [SMILES: O=[S@]1CCCCc2nc(N3CCN(c4ncc(Cl)cn4)CC3)nc(NC3(CO)CCC3)c21]

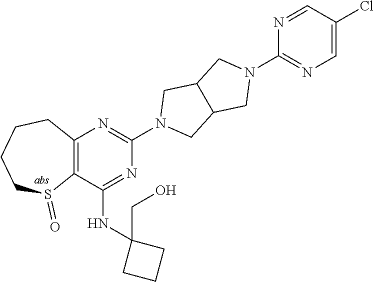 [SMILES: O=[S@]1CCCCc2nc(N3CC4CN(c5ncc(Cl)cn5)CC4C3)nc(NC3(CO)CCC3)c21]

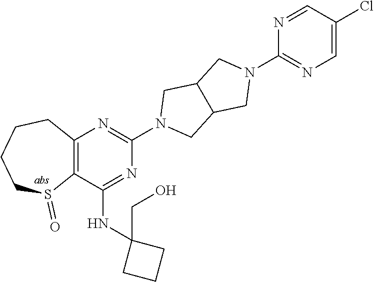 [SMILES: O=[S@]1CCCCc2nc(N3CC4CN(c5ncc(Cl)cn5)CC4C3)nc(NC3(CO)CCC3)c21]

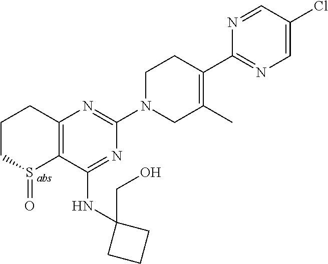 [SMILES: CC1=C(c2ncc(Cl)cn2)CCN(c2nc3c(c(NC4(CO)CCC4)n2)[S@](=O)CCC3)C1]

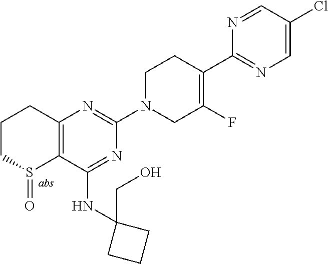 [SMILES: O=[S@@]1CCCc2nc(N3CCC(c4ncc(Cl)cn4)=C(F)C3)nc(NC3(CO)CCC3)c21]

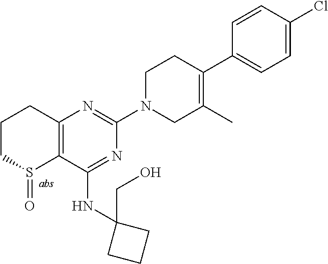 [SMILES: CC1=C(c2ccc(Cl)cc2)CCN(c2nc3c(c(NC4(CO)CCC4)n2)[S@](=O)CCC3)C1]

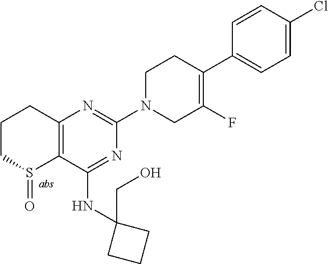 [SMILES: O=[S@@]1CCCc2nc(N3CCC(c4ccc(Cl)cc4)=C(F)C3)nc(NC3(CO)CCC3)c21]

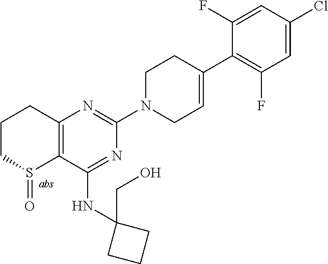 [SMILES: O=[S@@]1CCCc2nc(N3CC=C(c4c(F)cc(Cl)cc4F)CC3)nc(NC3(CO)CCC3)c21]

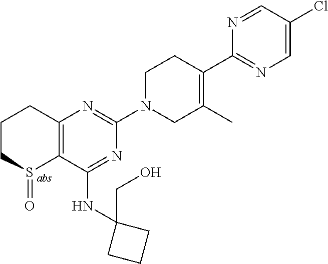 [SMILES: CC1=C(c2ncc(Cl)cn2)CCN(c2nc3c(c(NC4(CO)CCC4)n2)[S@@](=O)CCC3)C1]

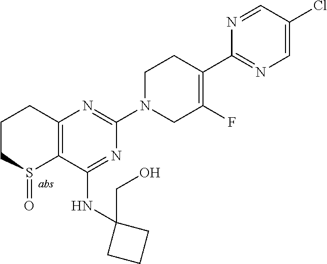 [SMILES: O=[S@]1CCCc2nc(N3CCC(c4ncc(Cl)cn4)=C(F)C3)nc(NC3(CO)CCC3)c21]

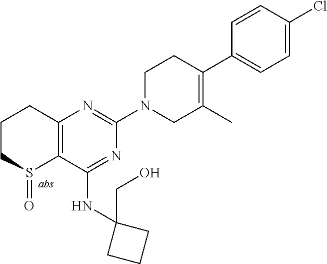 [SMILES: CC1=C(c2ccc(Cl)cc2)CCN(c2nc3c(c(NC4(CO)CCC4)n2)[S@@](=O)CCC3)C1]

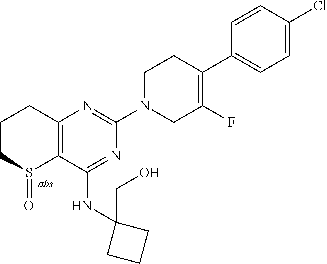 [SMILES: O=[S@]1CCCc2nc(N3CCC(c4ccc(Cl)cc4)=C(F)C3)nc(NC3(CO)CCC3)c21]

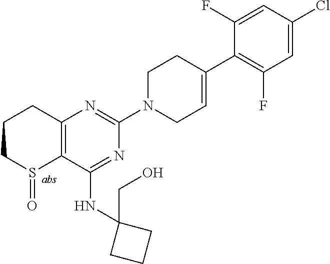

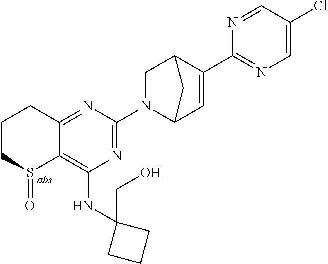 [SMILES: O=[S@]1CCCc2nc(N3CC4CC3C=C4c3ncc(Cl)cn3)nc(NC3(CO)CCC3)c21]

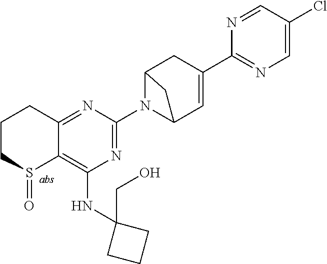 [SMILES: O=[S@]1CCCc2nc(N3C4C=C(c5ncc(Cl)cn5)CC3C4)nc(NC3(CO)CCC3)c21]

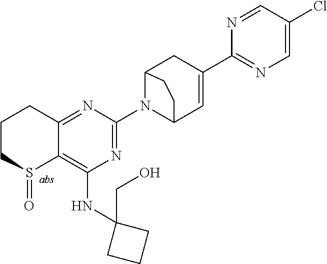 [SMILES: O=[S@]1CCCc2nc(N3C4C=C(c5ncc(Cl)cn5)CC3CC4)nc(NC3(CO)CCC3)c21]

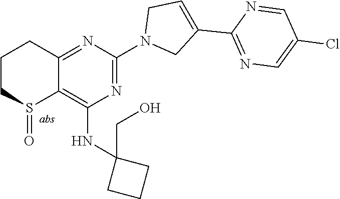 [SMILES: O=[S@]1CCCc2nc(N3CC=C(c4ncc(Cl)cn4)C3)nc(NC3(CO)CCC3)c21]

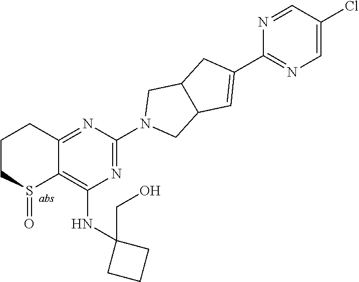 [SMILES: O=[S@]1CCCc2nc(N3CC4C=C(c5ncc(Cl)cn5)CC4C3)nc(NC3(CO)CCC3)c21]

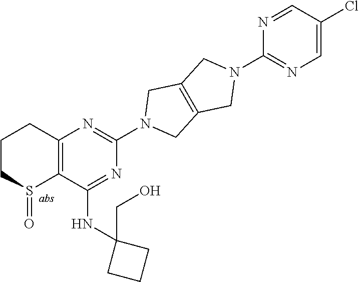 [SMILES: O=[S@]1CCCc2nc(N3CC4=C(CN(c5ncc(Cl)cn5)C4)C3)nc(NC3(CO)CCC3)c21]

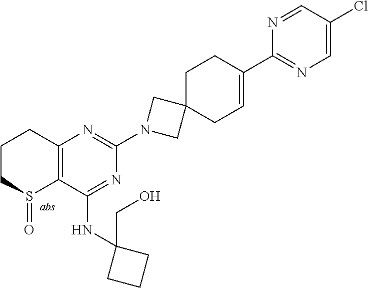 [SMILES: O=[S@]1CCCc2nc(N3CC4(CC=C(c5ncc(Cl)cn5)CC4)C3)nc(NC3(CO)CCC3)c21]

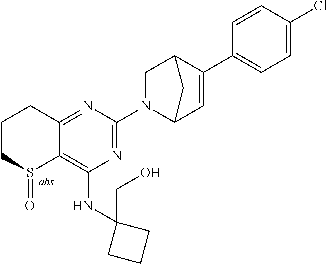 [SMILES: O=[S@]1CCCc2nc(N3CC4CC3C=C4c3ccc(Cl)cc3)nc(NC3(CO)CCC3)c21]

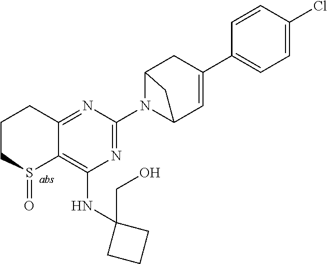 [SMILES: O=[S@]1CCCc2nc(N3C4C=C(c5ccc(Cl)cc5)CC3C4)nc(NC3(CO)CCC3)c21]

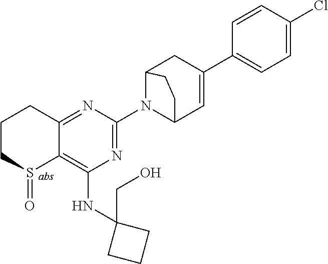 [SMILES: O=[S@]1CCCc2nc(N3C4C=C(c5ccc(Cl)cc5)CC3CC4)nc(NC3(CO)CCC3)c21]

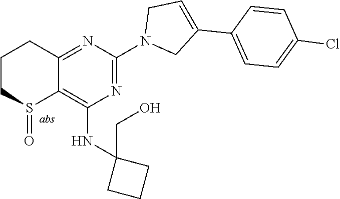 [SMILES: O=[S@]1CCCc2nc(N3CC=C(c4ccc(Cl)cc4)C3)nc(NC3(CO)CCC3)c21]

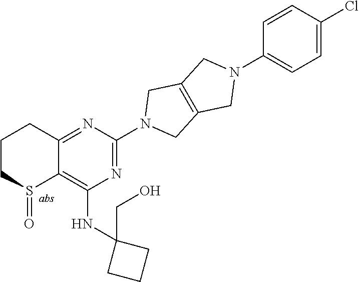 [SMILES: O=[S@]1CCCc2nc(N3CC4=C(CN(c5ccc(Cl)cc5)C4)C3)nc(NC3(CO)CCC3)c21]

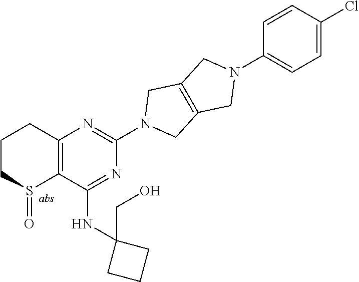 [SMILES: O=[S@]1CCCc2nc(N3CC4=C(CN(c5ccc(Cl)cc5)C4)C3)nc(NC3(CO)CCC3)c21]

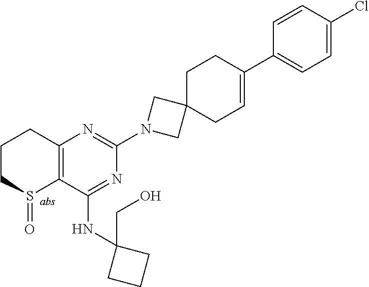 [SMILES: O=[S@]1CCCc2nc(N3CC4(CC=C(c5ccc(Cl)cc5)CC4)C3)nc(NC3(CO)CCC3)c21]

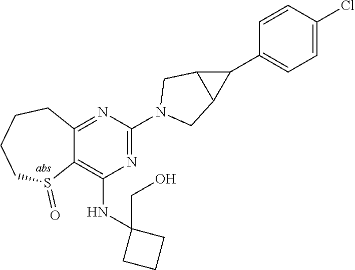 [SMILES: O=[S@@]1CCCCc2nc(N3CC4C(C3)C4c3ccc(Cl)cc3)nc(NC3(CO)CCC3)c21]

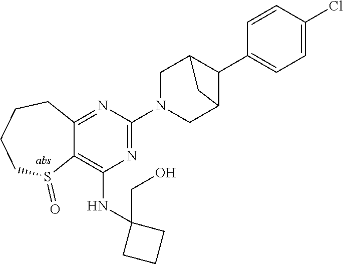 [SMILES: O=[S@@]1CCCCc2nc(N3CC4CC(C3)C4c3ccc(Cl)cc3)nc(NC3(CO)CCC3)c21]

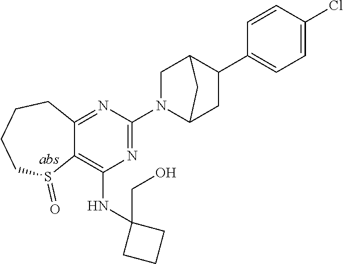 [SMILES: O=[S@@]1CCCCc2nc(N3CC4CC3CC4c3ccc(Cl)cc3)nc(NC3(CO)CCC3)c21]

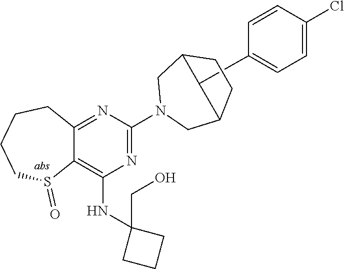 [SMILES: O=[S@@]1CCCCc2nc(N3CC4CCC(C3)C4c3ccc(Cl)cc3)nc(NC3(CO)CCC3)c21]

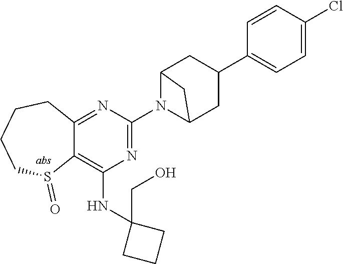 [SMILES: O=[S@@]1CCCCc2nc(N3C4CC(c5ccc(Cl)cc5)CC3C4)nc(NC3(CO)CCC3)c21]

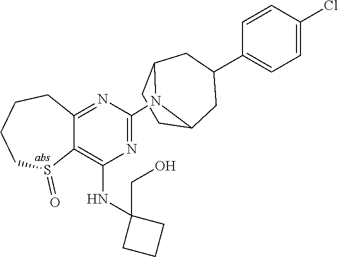 [SMILES: O=[S@@]1CCCCc2nc(N3C4CCC3CC(c3ccc(Cl)cc3)C4)nc(NC3(CO)CCC3)c21]

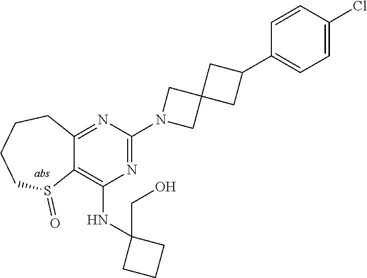 [SMILES: O=[S@@]1CCCCc2nc(N3CC4(CC(c5ccc(Cl)cc5)C4)C3)nc(NC3(CO)CCC3)c21]

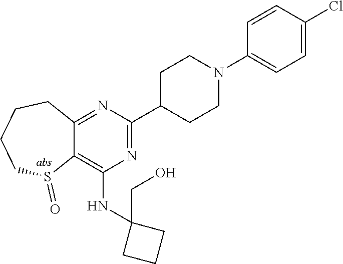 [SMILES: O=[S@@]1CCCCc2nc(C3CCN(c4ccc(Cl)cc4)CC3)nc(NC3(CO)CCC3)c21]

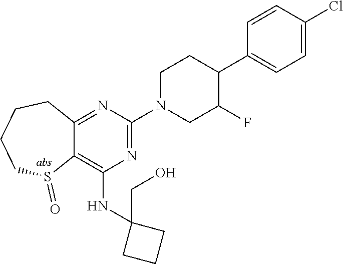 [SMILES: O=[S@@]1CCCCc2nc(N3CCC(c4ccc(Cl)cc4)C(F)C3)nc(NC3(CO)CCC3)c21]

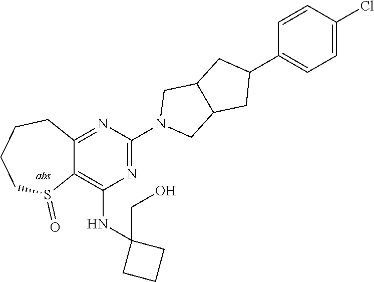 [SMILES: O=[S@@]1CCCCc2nc(N3CC4CC(c5ccc(Cl)cc5)CC4C3)nc(NC3(CO)CCC3)c21]

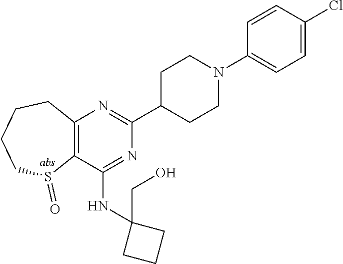 [SMILES: O=[S@@]1CCCCc2nc(C3CCN(c4ccc(Cl)cc4)CC3)nc(NC3(CO)CCC3)c21]

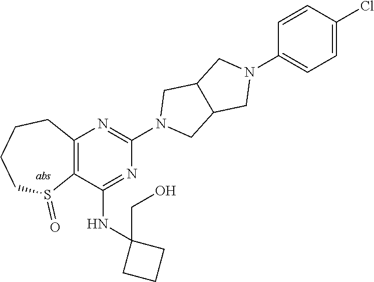 [SMILES: O=[S@@]1CCCCc2nc(N3CC4CN(c5ccc(Cl)cc5)CC4C3)nc(NC3(CO)CCC3)c21]

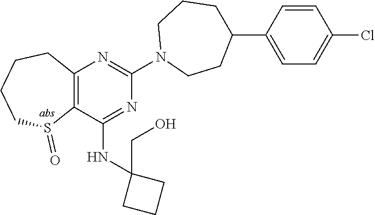 [SMILES: O=[S@@]1CCCCc2nc(N3CCCC(c4ccc(Cl)cc4)CC3)nc(NC3(CO)CCC3)c21]

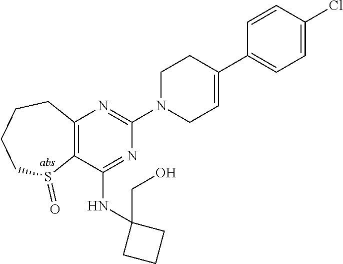 [SMILES: O=[S@@]1CCCCc2nc(N3CC=C(c4ccc(Cl)cc4)CC3)nc(NC3(CO)CCC3)c21]

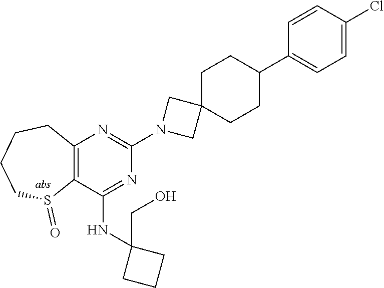 [SMILES: O=[S@@]1CCCCc2nc(N3CC4(CCC(c5ccc(Cl)cc5)CC4)C3)nc(NC3(CO)CCC3)c21]

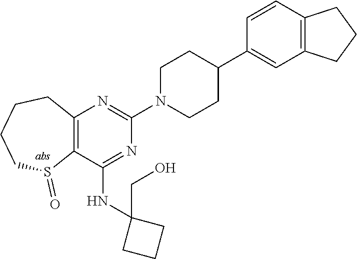 [SMILES: O=[S@@]1CCCCc2nc(N3CCC(c4ccc5c(c4)CCC5)CC3)nc(NC3(CO)CCC3)c21]

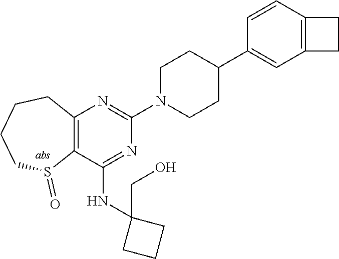 [SMILES: O=[S@@]1CCCCc2nc(N3CCC(c4ccc5c(c4)CC5)CC3)nc(NC3(CO)CCC3)c21]

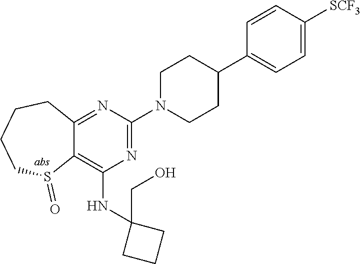

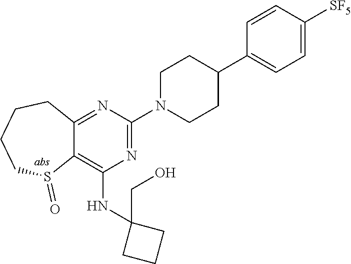

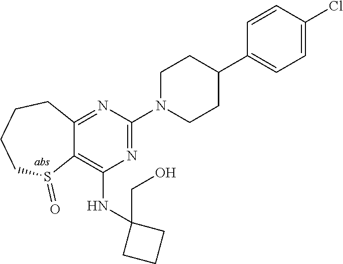 [SMILES: O=[S@@]1CCCCc2nc(N3CCC(c4ccc(Cl)cc4)CC3)nc(NC3(CO)CCC3)c21]

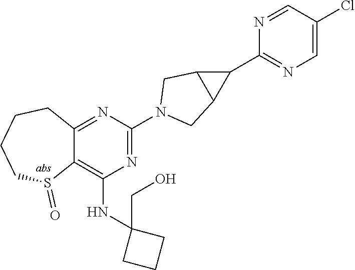 [SMILES: O=[S@@]1CCCCc2nc(N3CC4C(C3)C4c3ncc(Cl)cn3)nc(NC3(CO)CCC3)c21]

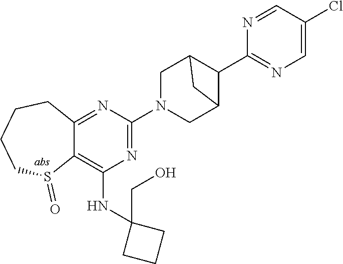 [SMILES: O=[S@@]1CCCCc2nc(N3CC4CC(C3)C4c3ncc(Cl)cn3)nc(NC3(CO)CCC3)c21]

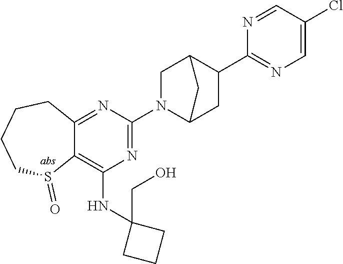 [SMILES: O=[S@@]1CCCCc2nc(N3CC4CC3CC4c3ncc(Cl)cn3)nc(NC3(CO)CCC3)c21]

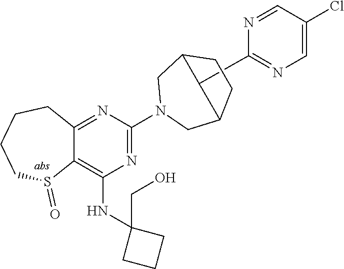 [SMILES: O=[S@@]1CCCCc2nc(N3CC4CCC(C3)C4c3ncc(Cl)cn3)nc(NC3(CO)CCC3)c21]

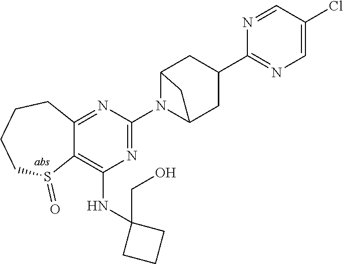 [SMILES: O=[S@@]1CCCCc2nc(N3C4CC(c5ncc(Cl)cn5)CC3C4)nc(NC3(CO)CCC3)c21]

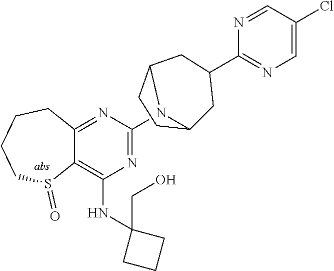 [SMILES: O=[S@@]1CCCCc2nc(N3C4CCC3CC(c3ncc(Cl)cn3)C4)nc(NC3(CO)CCC3)c21]

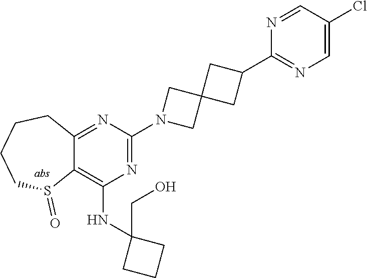 [SMILES: O=[S@@]1CCCCc2nc(N3CC4(CC(c5ncc(Cl)cn5)C4)C3)nc(NC3(CO)CCC3)c21]

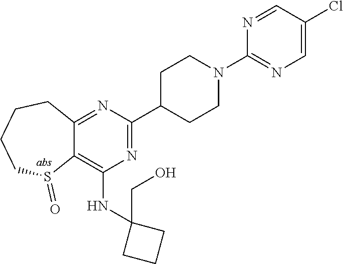 [SMILES: O=[S@@]1CCCCc2nc(C3CCN(c4ncc(Cl)cn4)CC3)nc(NC3(CO)CCC3)c21]

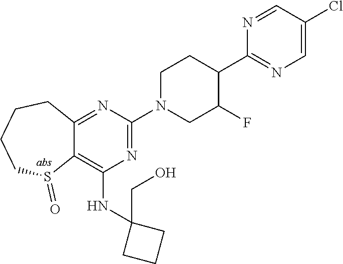 [SMILES: O=[S@@]1CCCCc2nc(N3CCC(c4ncc(Cl)cn4)C(F)C3)nc(NC3(CO)CCC3)c21]

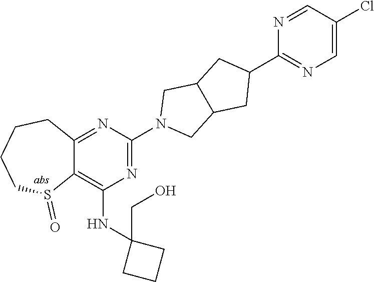 [SMILES: O=[S@@]1CCCCc2nc(N3CC4CC(c5ncc(Cl)cn5)CC4C3)nc(NC3(CO)CCC3)c21]

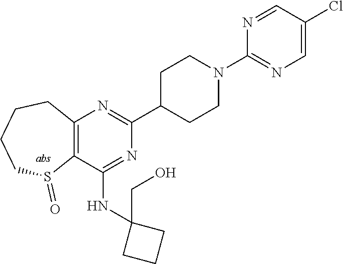 [SMILES: O=[S@@]1CCCCc2nc(C3CCN(c4ncc(Cl)cn4)CC3)nc(NC3(CO)CCC3)c21]

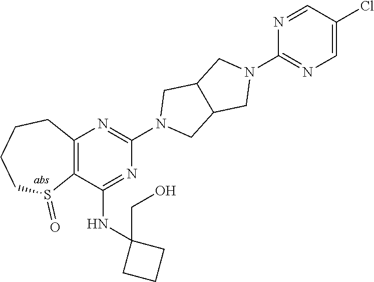 [SMILES: O=[S@@]1CCCCc2nc(N3CC4CN(c5ncc(Cl)cn5)CC4C3)nc(NC3(CO)CCC3)c21]

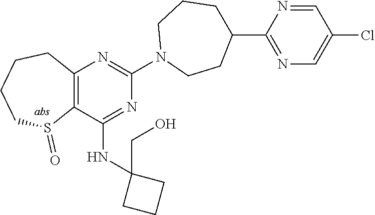 [SMILES: O=[S@@]1CCCCc2nc(N3CCCC(c4ncc(Cl)cn4)CC3)nc(NC3(CO)CCC3)c21]

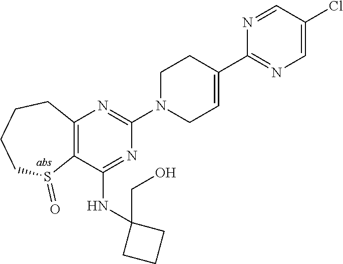 [SMILES: O=[S@@]1CCCCc2nc(N3CC=C(c4ncc(Cl)cn4)CC3)nc(NC3(CO)CCC3)c21]

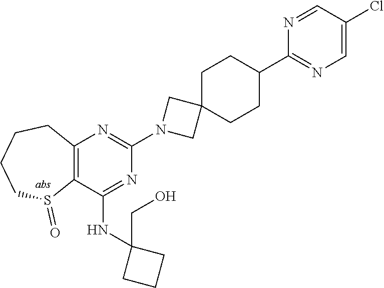 [SMILES: O=[S@@]1CCCCc2nc(N3CC4(CCC(c5ncc(Cl)cn5)CC4)C3)nc(NC3(CO)CCC3)c21]

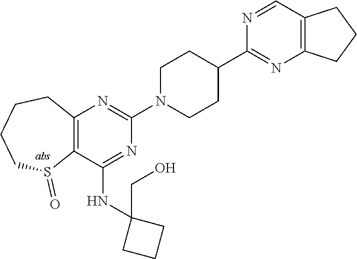 [SMILES: O=[S@@]1CCCCc2nc(N3CCC(c4ncc5c(n4)CCC5)CC3)nc(NC3(CO)CCC3)c21]

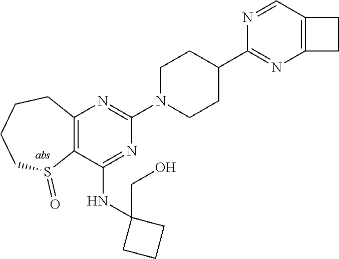 [SMILES: O=[S@@]1CCCCc2nc(N3CCC(c4ncc5c(n4)CC5)CC3)nc(NC3(CO)CCC3)c21]

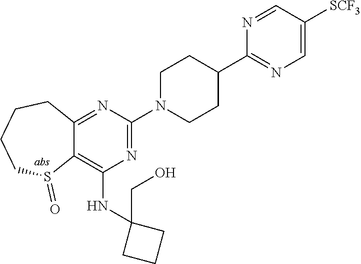

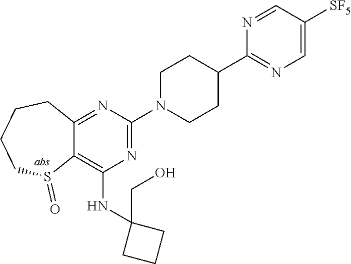

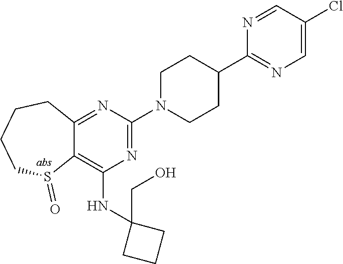 [SMILES: O=[S@@]1CCCCc2nc(N3CCC(c4ncc(Cl)cn4)CC3)nc(NC3(CO)CCC3)c21]

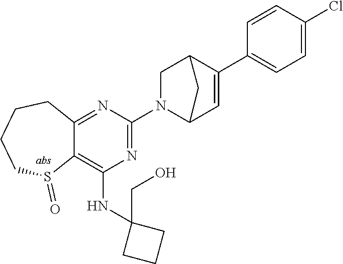 [SMILES: O=[S@@]1CCCCc2nc(N3CC4CC3C=C4c3ccc(Cl)cc3)nc(NC3(CO)CCC3)c21]

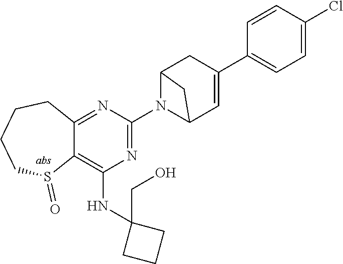 [SMILES: O=[S@@]1CCCCc2nc(N3C4C=C(c5ccc(Cl)cc5)CC3C4)nc(NC3(CO)CCC3)c21]

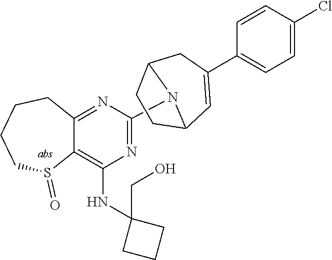 [SMILES: O=[S@@]1CCCCc2nc(N3C4C=C(c5ccc(Cl)cc5)CC3CC4)nc(NC3(CO)CCC3)c21]

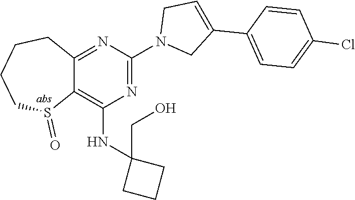 [SMILES: O=[S@@]1CCCCc2nc(N3CC=C(c4ccc(Cl)cc4)C3)nc(NC3(CO)CCC3)c21]

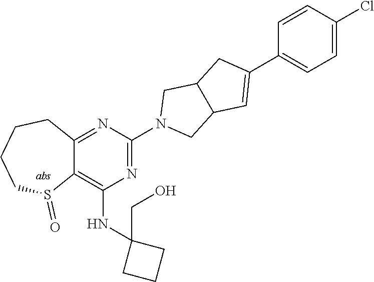 [SMILES: O=[S@@]1CCCCc2nc(N3CC4C=C(c5ccc(Cl)cc5)CC4C3)nc(NC3(CO)CCC3)c21]

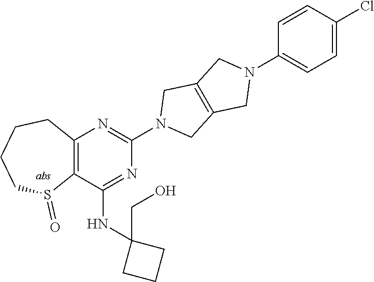 [SMILES: O=[S@@]1CCCCc2nc(N3CC4=C(CN(c5ccc(Cl)cc5)C4)C3)nc(NC3(CO)CCC3)c21]

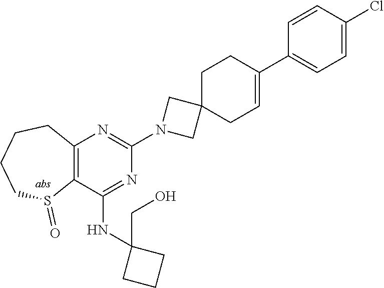 [SMILES: O=[S@@]1CCCCc2nc(N3CC4(CC=C(c5ccc(Cl)cc5)CC4)C3)nc(NC3(CO)CCC3)c21]

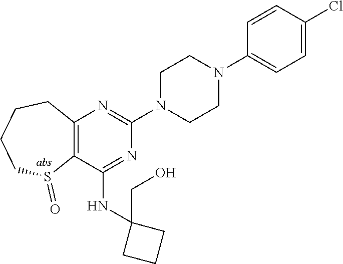 [SMILES: O=[S@@]1CCCCc2nc(N3CCN(c4ccc(Cl)cc4)CC3)nc(NC3(CO)CCC3)c21]

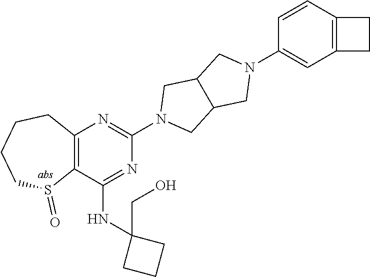 [SMILES: O=[S@@]1CCCCc2nc(N3CC4CN(c5ccc6c(c5)CC6)CC4C3)nc(NC3(CO)CCC3)c21]

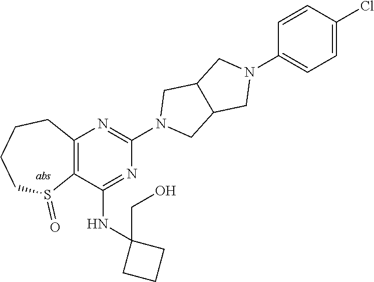 [SMILES: O=[S@@]1CCCCc2nc(N3CC4CN(c5ccc(Cl)cc5)CC4C3)nc(NC3(CO)CCC3)c21]

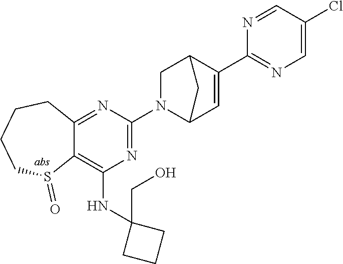 [SMILES: O=[S@@]1CCCCc2nc(N3CC4CC3C=C4c3ncc(Cl)cn3)nc(NC3(CO)CCC3)c21]

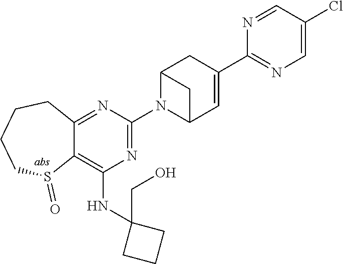 [SMILES: O=[S@@]1CCCCc2nc(N3C4C=C(c5ncc(Cl)cn5)CC3C4)nc(NC3(CO)CCC3)c21]

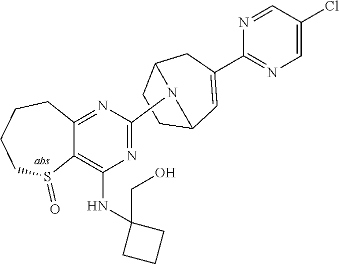 [SMILES: O=[S@@]1CCCCc2nc(N3C4C=C(c5ncc(Cl)cn5)CC3CC4)nc(NC3(CO)CCC3)c21]

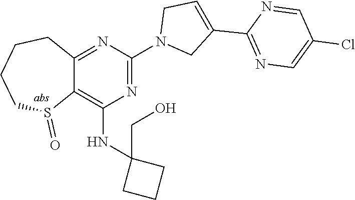 [SMILES: O=[S@@]1CCCCc2nc(N3CC=C(c4ncc(Cl)cn4)C3)nc(NC3(CO)CCC3)c21]

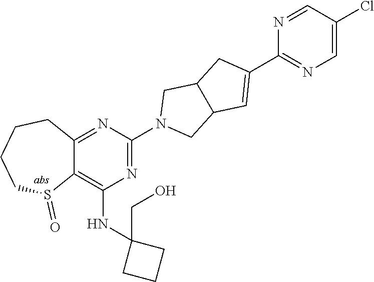 [SMILES: O=[S@@]1CCCCc2nc(N3CC4C=C(c5ncc(Cl)cn5)CC4C3)nc(NC3(CO)CCC3)c21]

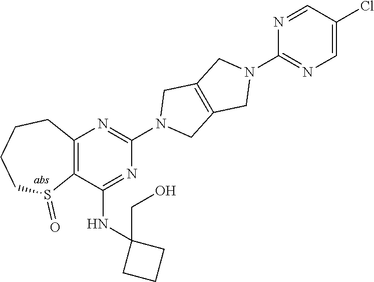 [SMILES: O=[S@@]1CCCCc2nc(N3CC4=C(CN(c5ncc(Cl)cn5)C4)C3)nc(NC3(CO)CCC3)c21]

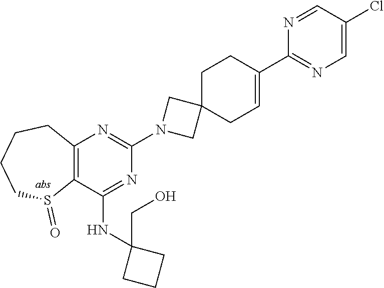 [SMILES: O=[S@@]1CCCCc2nc(N3CC4(CC=C(c5ncc(Cl)cn5)CC4)C3)nc(NC3(CO)CCC3)c21]

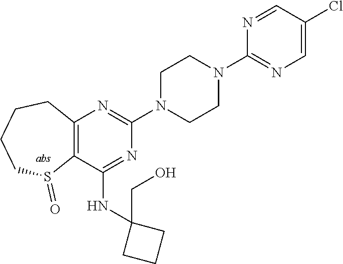 [SMILES: O=[S@@]1CCCCc2nc(N3CCN(c4ncc(Cl)cn4)CC3)nc(NC3(CO)CCC3)c21]

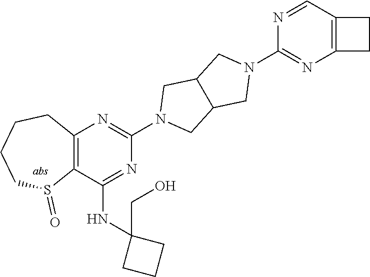 [SMILES: O=[S@@]1CCCCc2nc(N3CC4CN(c5ncc6c(n5)CC6)CC4C3)nc(NC3(CO)CCC3)c21]

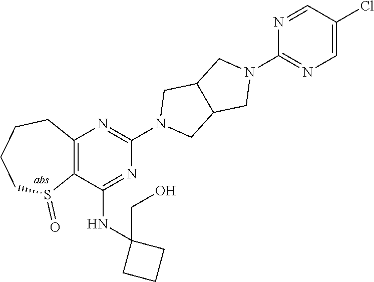 [SMILES: O=[S@@]1CCCCc2nc(N3CC4CN(c5ncc(Cl)cn5)CC4C3)nc(NC3(CO)CCC3)c21]

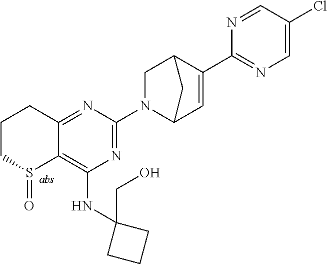 [SMILES: O=[S@@]1CCCc2nc(N3CC4CC3C=C4c3ncc(Cl)cn3)nc(NC3(CO)CCC3)c21]

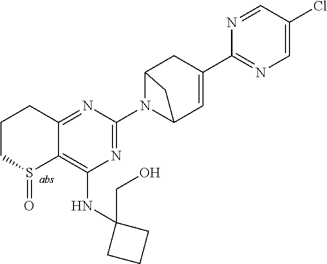 [SMILES: O=[S@@]1CCCc2nc(N3C4C=C(c5ncc(Cl)cn5)CC3C4)nc(NC3(CO)CCC3)c21]

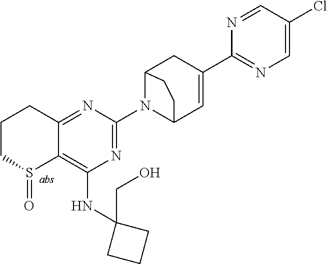 [SMILES: O=[S@@]1CCCc2nc(N3C4C=C(c5ncc(Cl)cn5)CC3CC4)nc(NC3(CO)CCC3)c21]

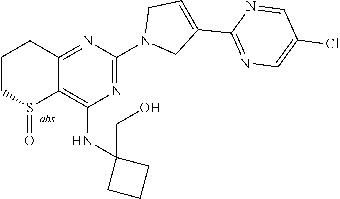 [SMILES: O=[S@@]1CCCc2nc(N3CC=C(c4ncc(Cl)cn4)C3)nc(NC3(CO)CCC3)c21]

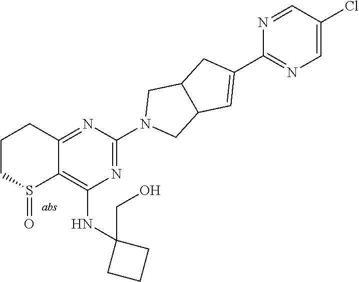 [SMILES: O=[S@@]1CCCc2nc(N3CC4C=C(c5ncc(Cl)cn5)CC4C3)nc(NC3(CO)CCC3)c21]

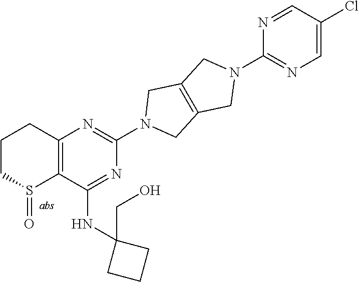 [SMILES: O=[S@@]1CCCc2nc(N3CC4=C(CN(c5ncc(Cl)cn5)C4)C3)nc(NC3(CO)CCC3)c21]

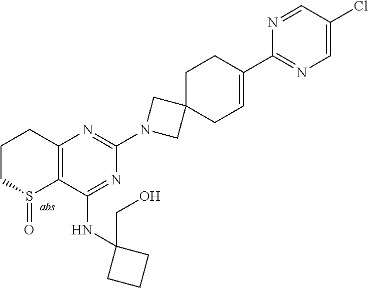 [SMILES: O=[S@@]1CCCc2nc(N3CC4(CC=C(c5ncc(Cl)cn5)CC4)C3)nc(NC3(CO)CCC3)c21]

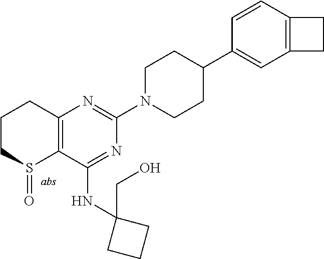 [SMILES: O=[S@]1CCCc2nc(N3CCC(c4ccc5c(c4)CC5)CC3)nc(NC3(CO)CCC3)c21]

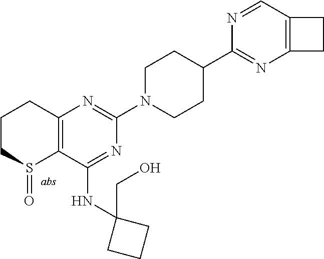 [SMILES: O=[S@]1CCCc2nc(N3CCC(c4ncc5c(n4)CC5)CC3)nc(NC3(CO)CCC3)c21]

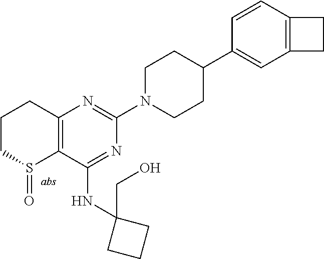 [SMILES: O=[S@@]1CCCc2nc(N3CCC(c4ccc5c(c4)CC5)CC3)nc(NC3(CO)CCC3)c21]

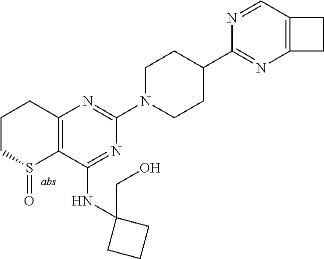 [SMILES: O=[S@@]1CCCc2nc(N3CCC(c4ncc5c(n4)CC5)CC3)nc(NC3(CO)CCC3)c21]

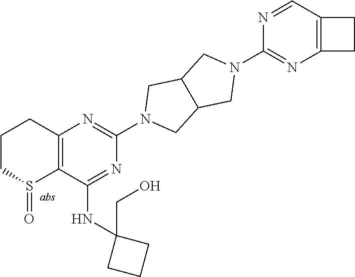 [SMILES: O=[S@@]1CCCc2nc(N3CC4CN(c5ncc6c(n5)CC6)CC4C3)nc(NC3(CO)CCC3)c21]

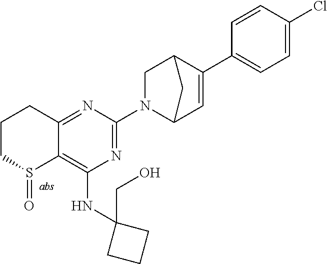 [SMILES: O=[S@@]1CCCc2nc(N3CC4CC3C=C4c3ccc(Cl)cc3)nc(NC3(CO)CCC3)c21]

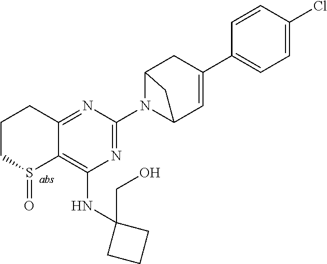 [SMILES: O=[S@@]1CCCc2nc(N3C4C=C(c5ccc(Cl)cc5)CC3C4)nc(NC3(CO)CCC3)c21]

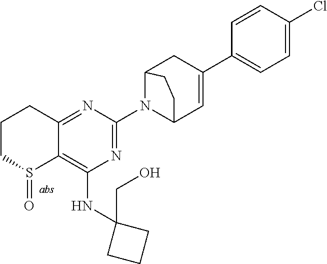 [SMILES: O=[S@@]1CCCc2nc(N3C4C=C(c5ccc(Cl)cc5)CC3CC4)nc(NC3(CO)CCC3)c21]

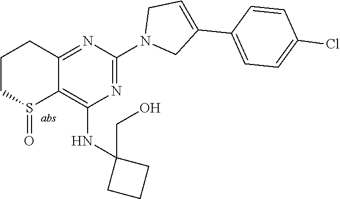 [SMILES: O=[S@@]1CCCc2nc(N3CC=C(c4ccc(Cl)cc4)C3)nc(NC3(CO)CCC3)c21]

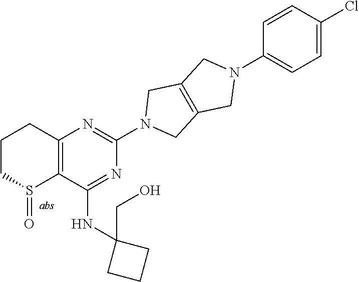 [SMILES: O=[S@@]1CCCc2nc(N3CC4=C(CN(c5ccc(Cl)cc5)C4)C3)nc(NC3(CO)CCC3)c21]

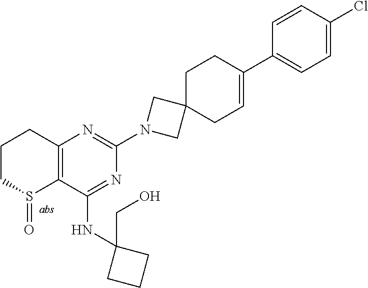 [SMILES: O=[S@@]1CCCc2nc(N3CC4(CC=C(c5ccc(Cl)cc5)CC4)C3)nc(NC3(CO)CCC3)c21]

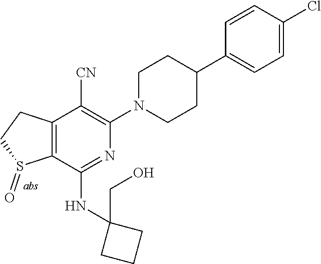 [SMILES: N#Cc1c(N2CCC(c3ccc(Cl)cc3)CC2)nc(NC2(CO)CCC2)c2c1CC[S@]2=O]

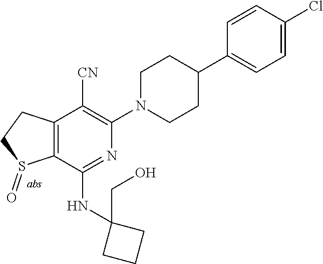 [SMILES: N#Cc1c(N2CCC(c3ccc(Cl)cc3)CC2)nc(NC2(CO)CCC2)c2c1CC[S@@]2=O]

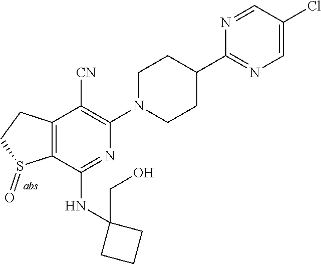 [SMILES: N#Cc1c(N2CCC(c3ncc(Cl)cn3)CC2)nc(NC2(CO)CCC2)c2c1CC[S@]2=O]

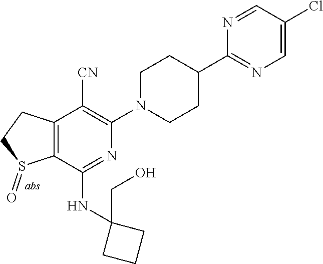 [SMILES: N#Cc1c(N2CCC(c3ncc(Cl)cn3)CC2)nc(NC2(CO)CCC2)c2c1CC[S@@]2=O]

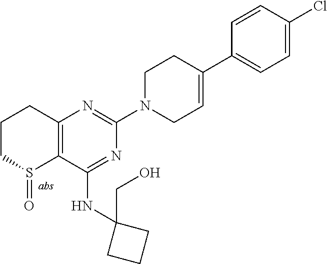 [SMILES: O=[S@@]1CCCc2nc(N3CC=C(c4ccc(Cl)cc4)CC3)nc(NC3(CO)CCC3)c21]

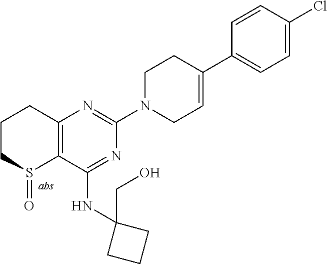 [SMILES: O=[S@]1CCCc2nc(N3CC=C(c4ccc(Cl)cc4)CC3)nc(NC3(CO)CCC3)c21]

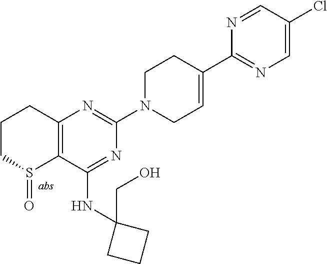 [SMILES: O=[S@@]1CCCc2nc(N3CC=C(c4ncc(Cl)cn4)CC3)nc(NC3(CO)CCC3)c21]

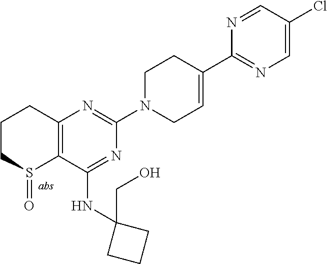 [SMILES: O=[S@]1CCCc2nc(N3CC=C(c4ncc(Cl)cn4)CC3)nc(NC3(CO)CCC3)c21]

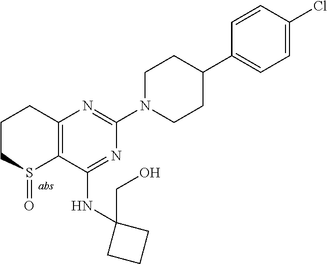 [SMILES: O=[S@]1CCCc2nc(N3CCC(c4ccc(Cl)cc4)CC3)nc(NC3(CO)CCC3)c21]

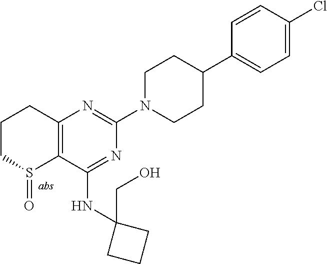 [SMILES: O=[S@@]1CCCc2nc(N3CCC(c4ccc(Cl)cc4)CC3)nc(NC3(CO)CCC3)c21]

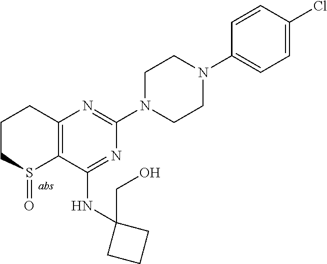 [SMILES: O=[S@]1CCCc2nc(N3CCN(c4ccc(Cl)cc4)CC3)nc(NC3(CO)CCC3)c21]

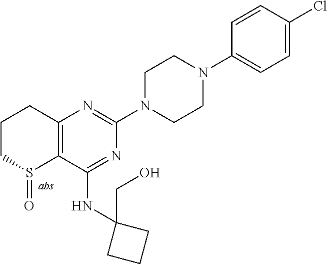 [SMILES: O=[S@@]1CCCc2nc(N3CCN(c4ccc(Cl)cc4)CC3)nc(NC3(CO)CCC3)c21]

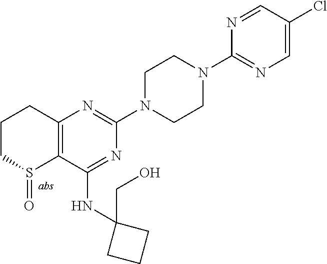 [SMILES: O=[S@@]1CCCc2nc(N3CCN(c4ncc(Cl)cn4)CC3)nc(NC3(CO)CCC3)c21]

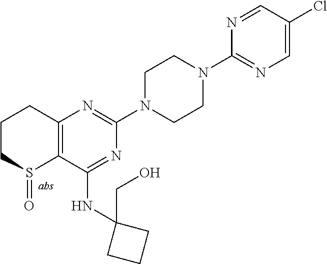 [SMILES: O=[S@]1CCCc2nc(N3CCN(c4ncc(Cl)cn4)CC3)nc(NC3(CO)CCC3)c21]

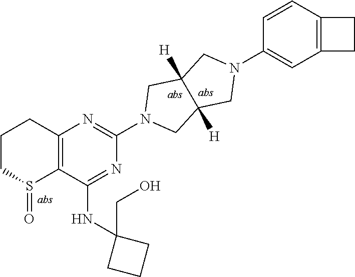 [SMILES: O=[S@@]1CCCc2nc(N3C[C@H]4CN(c5ccc6c(c5)CC6)C[C@H]4C3)nc(NC3(CO)CCC3)c21]

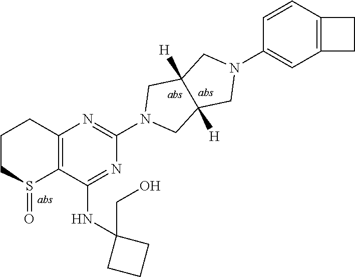 [SMILES: O=[S@]1CCCc2nc(N3C[C@H]4CN(c5ccc6c(c5)CC6)C[C@H]4C3)nc(NC3(CO)CCC3)c21]

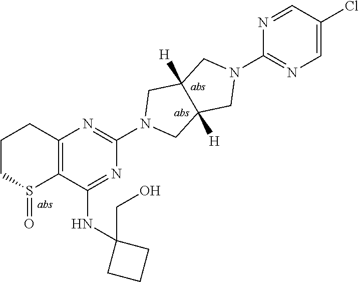 [SMILES: O=[S@@]1CCCc2nc(N3C[C@H]4CN(c5ncc(Cl)cn5)C[C@H]4C3)nc(NC3(CO)CCC3)c21]

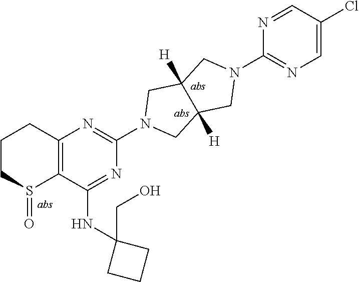 [SMILES: O=[S@]1CCCc2nc(N3C[C@H]4CN(c5ncc(Cl)cn5)C[C@H]4C3)nc(NC3(CO)CCC3)c21]

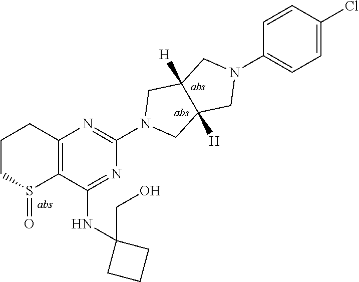 [SMILES: O=[S@@]1CCCc2nc(N3C[C@H]4CN(c5ccc(Cl)cc5)C[C@H]4C3)nc(NC3(CO)CCC3)c21]

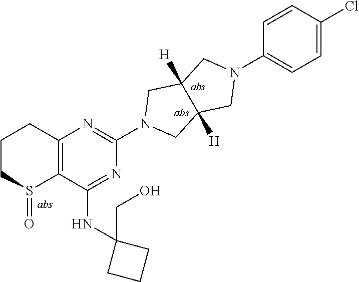 [SMILES: O=[S@]1CCCc2nc(N3C[C@H]4CN(c5ccc(Cl)cc5)C[C@H]4C3)nc(NC3(CO)CCC3)c21]

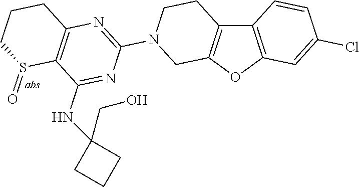 [SMILES: O=[S@@]1CCCc2nc(N3CCc4c(oc5cc(Cl)ccc45)C3)nc(NC3(CO)CCC3)c21]

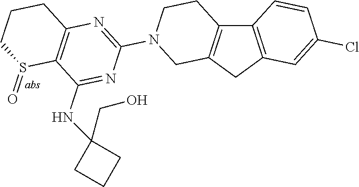 [SMILES: O=[S@@]1CCCc2nc(N3CCC4=C(Cc5cc(Cl)ccc54)C3)nc(NC3(CO)CCC3)c21]

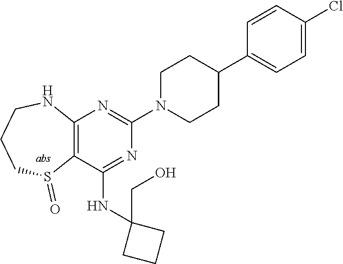 [SMILES: O=[S@@]1CCCNc2nc(N3CCC(c4ccc(Cl)cc4)CC3)nc(NC3(CO)CCC3)c21]

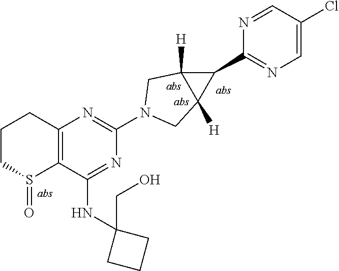 [SMILES: O=[S@@]1CCCc2nc(N3C[C@@H]4[C@H](C3)[C@H]4c3ncc(Cl)cn3)nc(NC3(CO)CCC3)c21]

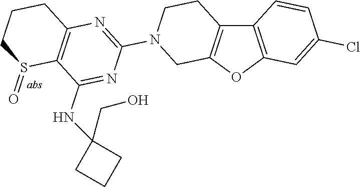 [SMILES: O=[S@]1CCCc2nc(N3CCc4c(oc5cc(Cl)ccc45)C3)nc(NC3(CO)CCC3)c21]

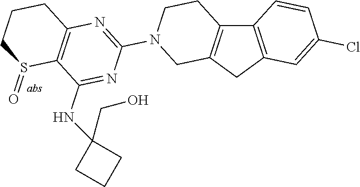 [SMILES: O=[S@]1CCCc2nc(N3CCC4=C(Cc5cc(Cl)ccc54)C3)nc(NC3(CO)CCC3)c21]

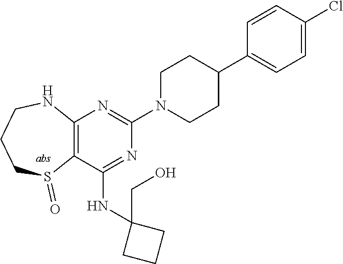 [SMILES: O=[S@]1CCCNc2nc(N3CCC(c4ccc(Cl)cc4)CC3)nc(NC3(CO)CCC3)c21]

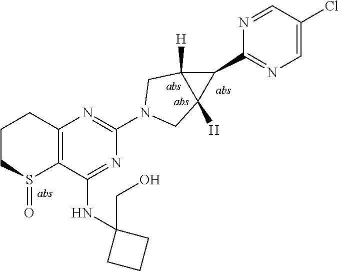 [SMILES: O=[S@]1CCCc2nc(N3C[C@@H]4[C@H](C3)[C@H]4c3ncc(Cl)cn3)nc(NC3(CO)CCC3)c21]

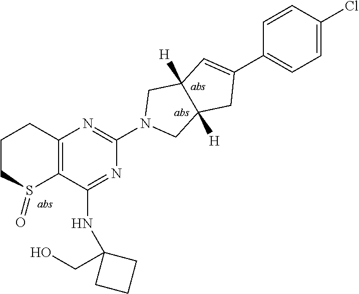 [SMILES: O=[S@]1CCCc2nc(N3C[C@H]4CC(c5ccc(Cl)cc5)=C[C@H]4C3)nc(NC3(CO)CCC3)c21]

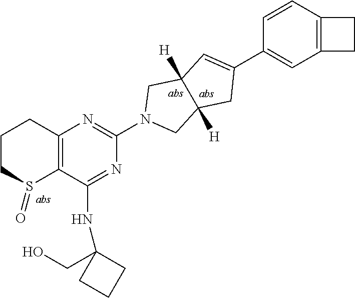 [SMILES: O=[S@]1CCCc2nc(N3C[C@H]4CC(c5ccc6c(c5)CC6)=C[C@H]4C3)nc(NC3(CO)CCC3)c21]

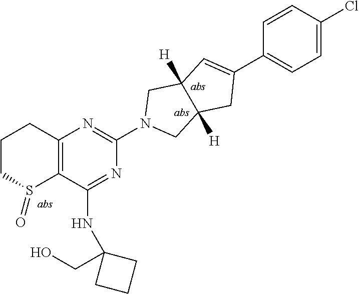 [SMILES: O=[S@@]1CCCc2nc(N3C[C@H]4CC(c5ccc(Cl)cc5)=C[C@H]4C3)nc(NC3(CO)CCC3)c21]

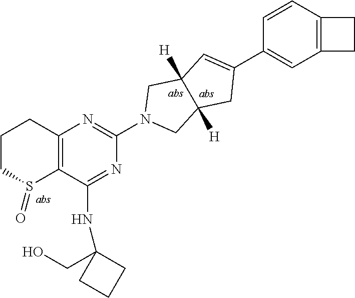 [SMILES: O=[S@@]1CCCc2nc(N3C[C@H]4CC(c5ccc6c(c5)CC6)=C[C@H]4C3)nc(NC3(CO)CCC3)c21]

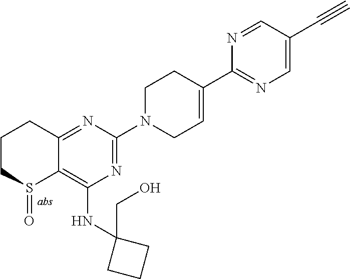 [SMILES: C#Cc1cnc(C2=CCN(c3nc4c(c(NC5(CO)CCC5)n3)[S@@](=O)CCC4)CC2)nc1]

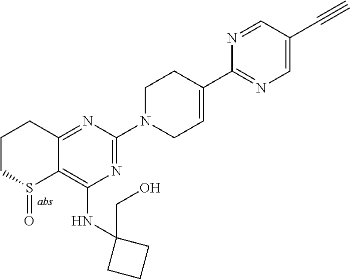 [SMILES: C#Cc1cnc(C2=CCN(c3nc4c(c(NC5(CO)CCC5)n3)[S@](=O)CCC4)CC2)nc1]

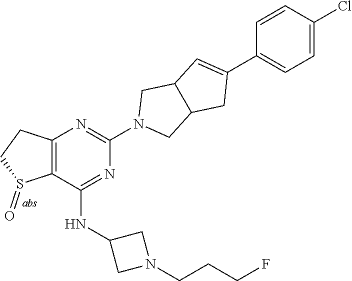 [SMILES: O=[S@@]1CCc2nc(N3CC4C=C(c5ccc(Cl)cc5)CC4C3)nc(NC3CN(CCCF)C3)c21]

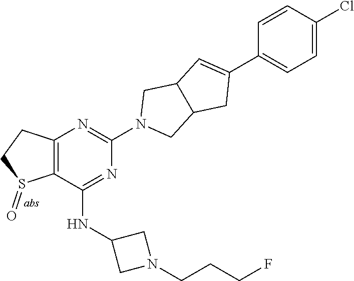 [SMILES: O=[S@]1CCc2nc(N3CC4C=C(c5ccc(Cl)cc5)CC4C3)nc(NC3CN(CCCF)C3)c21]

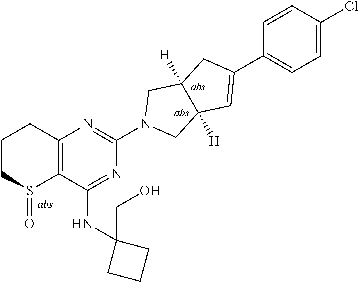 [SMILES: O=[S@]1CCCc2nc(N3C[C@H]4CC(c5ccc(Cl)cc5)=C[C@H]4C3)nc(NC3(CO)CCC3)c21]

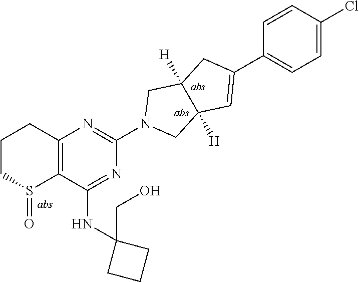 [SMILES: O=[S@@]1CCCc2nc(N3C[C@H]4CC(c5ccc(Cl)cc5)=C[C@H]4C3)nc(NC3(CO)CCC3)c21]

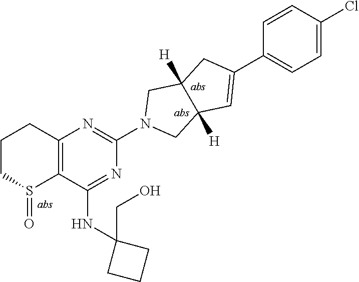 [SMILES: O=[S@@]1CCCc2nc(N3C[C@@H]4CC(c5ccc(Cl)cc5)=C[C@@H]4C3)nc(NC3(CO)CCC3)c21]

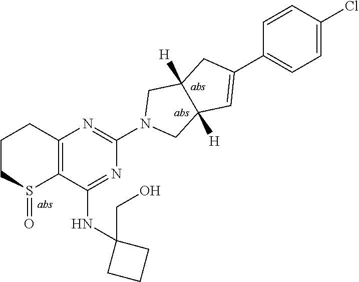 [SMILES: O=[S@]1CCCc2nc(N3C[C@@H]4CC(c5ccc(Cl)cc5)=C[C@@H]4C3)nc(NC3(CO)CCC3)c21]

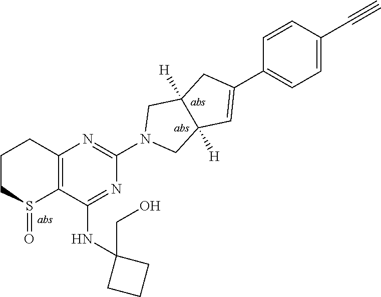 [SMILES: C#Cc1ccc(C2=C[C@H]3CN(c4nc5c(c(NC6(CO)CCC6)n4)[S@@](=O)CCC5)C[C@H]3C2)cc1]

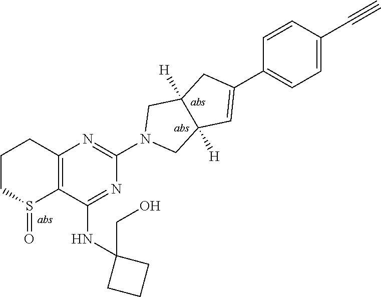 [SMILES: C#Cc1ccc(C2=C[C@H]3CN(c4nc5c(c(NC6(CO)CCC6)n4)[S@](=O)CCC5)C[C@H]3C2)cc1]

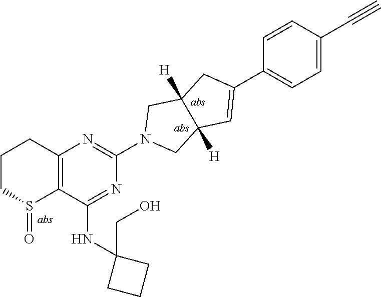 [SMILES: C#Cc1ccc(C2=C[C@@H]3CN(c4nc5c(c(NC6(CO)CCC6)n4)[S@](=O)CCC5)C[C@@H]3C2)cc1]

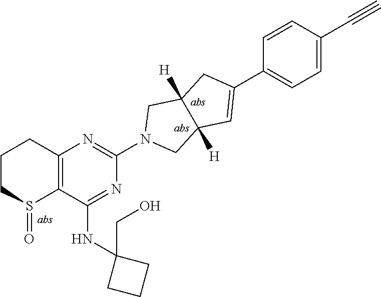 [SMILES: C#Cc1ccc(C2=C[C@@H]3CN(c4nc5c(c(NC6(CO)CCC6)n4)[S@@](=O)CCC5)C[C@@H]3C2)cc1]

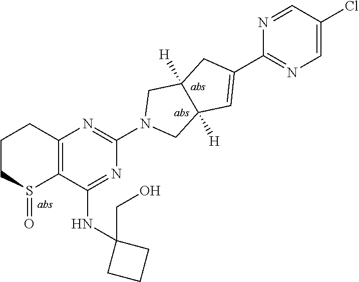 [SMILES: O=[S@]1CCCc2nc(N3C[C@H]4CC(c5ncc(Cl)cn5)=C[C@H]4C3)nc(NC3(CO)CCC3)c21]

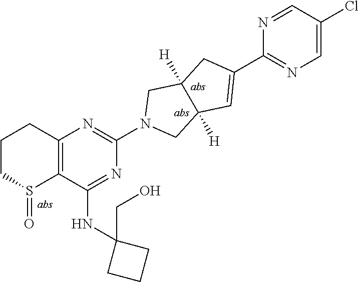 [SMILES: O=[S@@]1CCCc2nc(N3C[C@H]4CC(c5ncc(Cl)cn5)=C[C@H]4C3)nc(NC3(CO)CCC3)c21]

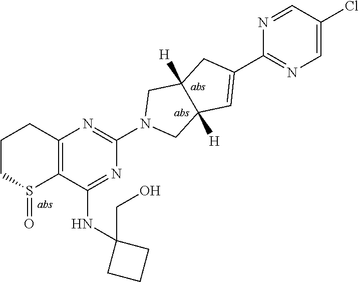 [SMILES: O=[S@@]1CCCc2nc(N3C[C@@H]4CC(c5ncc(Cl)cn5)=C[C@@H]4C3)nc(NC3(CO)CCC3)c21]

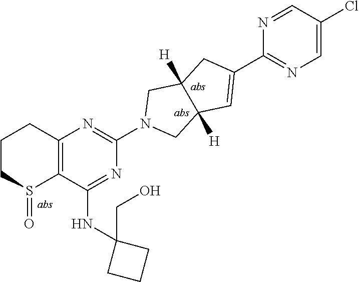 [SMILES: O=[S@]1CCCc2nc(N3C[C@@H]4CC(c5ncc(Cl)cn5)=C[C@@H]4C3)nc(NC3(CO)CCC3)c21]

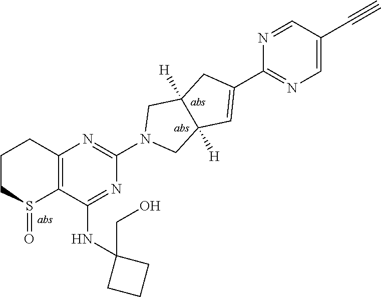 [SMILES: C#Cc1cnc(C2=C[C@H]3CN(c4nc5c(c(NC6(CO)CCC6)n4)[S@@](=O)CCC5)C[C@H]3C2)nc1]

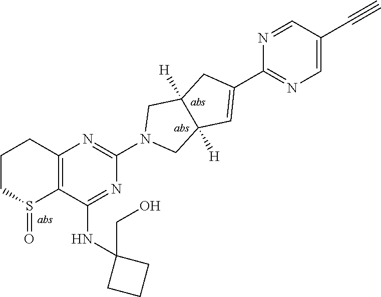 [SMILES: C#Cc1cnc(C2=C[C@H]3CN(c4nc5c(c(NC6(CO)CCC6)n4)[S@](=O)CCC5)C[C@H]3C2)nc1]

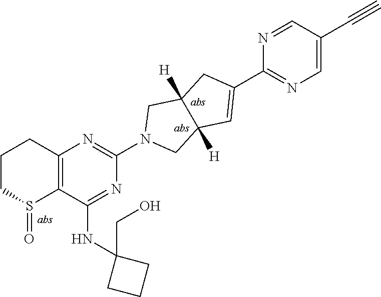 [SMILES: C#Cc1cnc(C2=C[C@@H]3CN(c4nc5c(c(NC6(CO)CCC6)n4)[S@](=O)CCC5)C[C@@H]3C2)nc1]

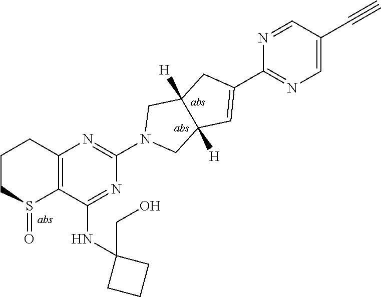 [SMILES: C#Cc1cnc(C2=C[C@@H]3CN(c4nc5c(c(NC6(CO)CCC6)n4)[S@@](=O)CCC5)C[C@@H]3C2)nc1]

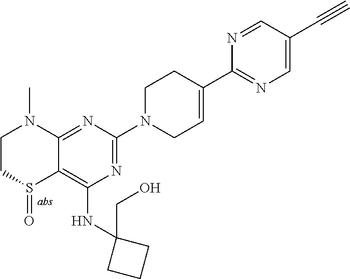 [SMILES: C#Cc1cnc(C2=CCN(c3nc(NC4(CO)CCC4)c4c(n3)N(C)CC[S@]4=O)CC2)nc1]

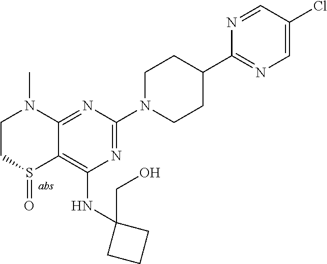 [SMILES: CN1CC[S@@](=O)c2c(NC3(CO)CCC3)nc(N3CCC(c4ncc(Cl)cn4)CC3)nc21]

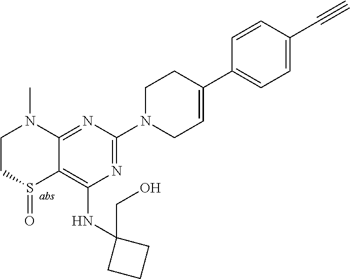 [SMILES: C#Cc1ccc(C2=CCN(c3nc(NC4(CO)CCC4)c4c(n3)N(C)CC[S@]4=O)CC2)cc1]

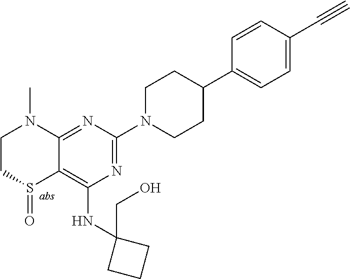 [SMILES: C#Cc1ccc(C2CCN(c3nc(NC4(CO)CCC4)c4c(n3)N(C)CC[S@]4=O)CC2)cc1]

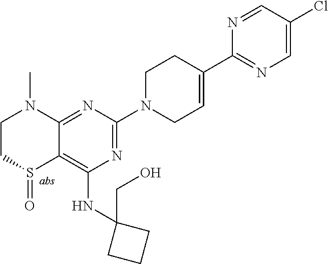 [SMILES: CN1CC[S@@](=O)c2c(NC3(CO)CCC3)nc(N3CC=C(c4ncc(Cl)cn4)CC3)nc21]

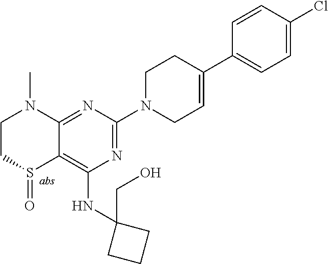 [SMILES: CN1CC[S@@](=O)c2c(NC3(CO)CCC3)nc(N3CC=C(c4ccc(Cl)cc4)CC3)nc21]

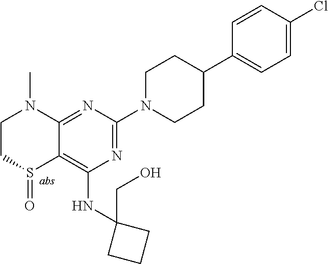 [SMILES: CN1CC[S@@](=O)c2c(NC3(CO)CCC3)nc(N3CCC(c4ccc(Cl)cc4)CC3)nc21]

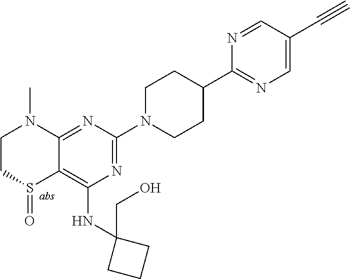 [SMILES: C#Cc1cnc(C2CCN(c3nc(NC4(CO)CCC4)c4c(n3)N(C)CC[S@]4=O)CC2)nc1]

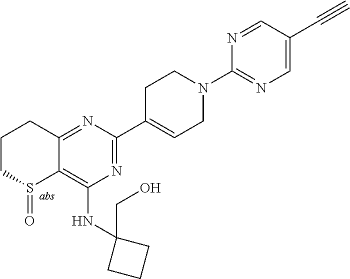 [SMILES: C#Cc1cnc(N2CC=C(c3nc4c(c(NC5(CO)CCC5)n3)[S@](=O)CCC4)CC2)nc1]

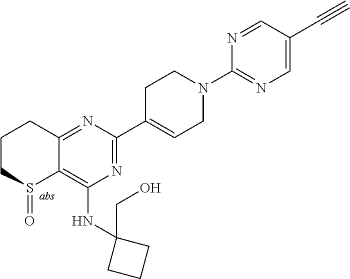 [SMILES: C#Cc1cnc(N2CC=C(c3nc4c(c(NC5(CO)CCC5)n3)[S@@](=O)CCC4)CC2)nc1]

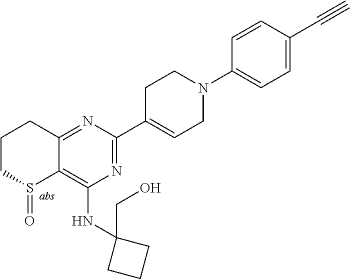 [SMILES: C#Cc1ccc(N2CC=C(c3nc4c(c(NC5(CO)CCC5)n3)[S@](=O)CCC4)CC2)cc1]

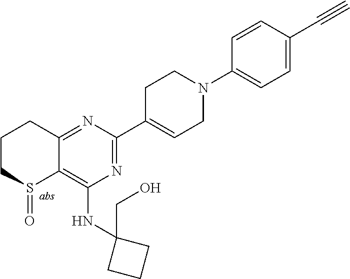 [SMILES: C#Cc1ccc(N2CC=C(c3nc4c(c(NC5(CO)CCC5)n3)[S@@](=O)CCC4)CC2)cc1]

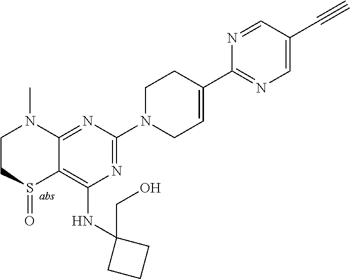 [SMILES: C#Cc1cnc(C2=CCN(c3nc(NC4(CO)CCC4)c4c(n3)N(C)CC[S@@]4=O)CC2)nc1]

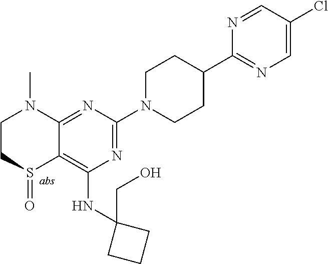 [SMILES: CN1CC[S@](=O)c2c(NC3(CO)CCC3)nc(N3CCC(c4ncc(Cl)cn4)CC3)nc21]

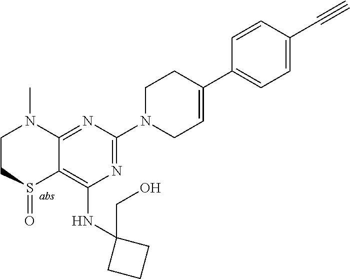 [SMILES: C#Cc1ccc(C2=CCN(c3nc(NC4(CO)CCC4)c4c(n3)N(C)CC[S@@]4=O)CC2)cc1]

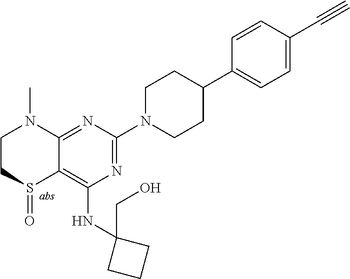 [SMILES: C#Cc1ccc(C2CCN(c3nc(NC4(CO)CCC4)c4c(n3)N(C)CC[S@@]4=O)CC2)cc1]

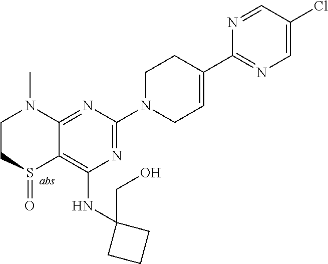 [SMILES: CN1CC[S@](=O)c2c(NC3(CO)CCC3)nc(N3CC=C(c4ncc(Cl)cn4)CC3)nc21]

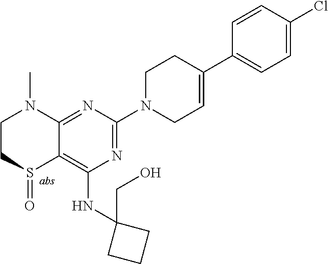 [SMILES: CN1CC[S@](=O)c2c(NC3(CO)CCC3)nc(N3CC=C(c4ccc(Cl)cc4)CC3)nc21]

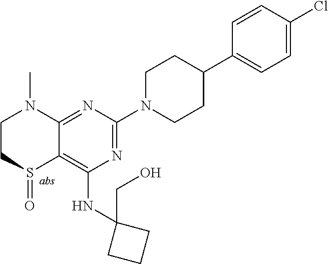 [SMILES: CN1CC[S@](=O)c2c(NC3(CO)CCC3)nc(N3CCC(c4ccc(Cl)cc4)CC3)nc21]

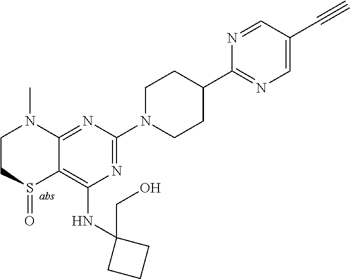 [SMILES: C#Cc1cnc(C2CCN(c3nc(NC4(CO)CCC4)c4c(n3)N(C)CC[S@@]4=O)CC2)nc1]

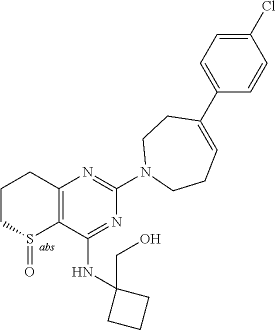 [SMILES: O=[S@@]1CCCc2nc(N3CCC=C(c4ccc(Cl)cc4)CC3)nc(NC3(CO)CCC3)c21]

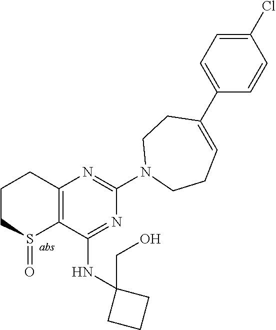 [SMILES: O=[S@]1CCCc2nc(N3CCC=C(c4ccc(Cl)cc4)CC3)nc(NC3(CO)CCC3)c21]

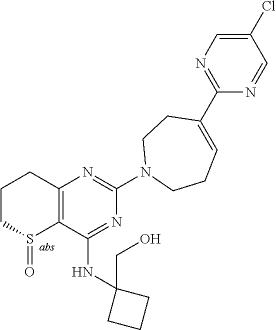 [SMILES: O=[S@@]1CCCc2nc(N3CCC=C(c4ncc(Cl)cn4)CC3)nc(NC3(CO)CCC3)c21]

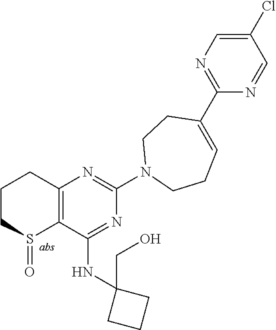 [SMILES: O=[S@]1CCCc2nc(N3CCC=C(c4ncc(Cl)cn4)CC3)nc(NC3(CO)CCC3)c21]

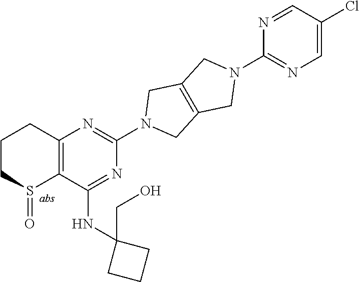 [SMILES: O=[S@]1CCCc2nc(N3CC4=C(CN(c5ncc(Cl)cn5)C4)C3)nc(NC3(CO)CCC3)c21]

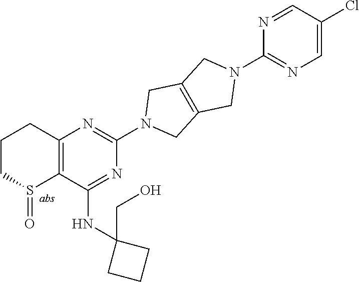 [SMILES: O=[S@@]1CCCc2nc(N3CC4=C(CN(c5ncc(Cl)cn5)C4)C3)nc(NC3(CO)CCC3)c21]

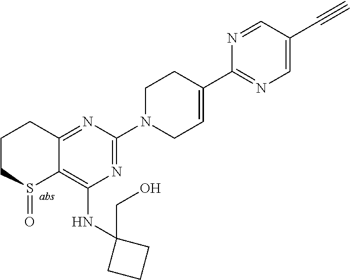 [SMILES: C#Cc1cnc(C2=CCN(c3nc4c(c(NC5(CO)CCC5)n3)[S@@](=O)CCC4)CC2)nc1]

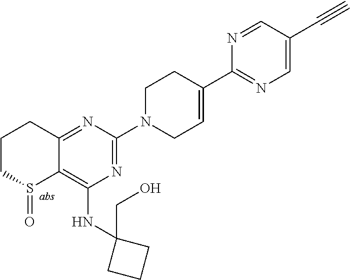 [SMILES: C#Cc1cnc(C2=CCN(c3nc4c(c(NC5(CO)CCC5)n3)[S@](=O)CCC4)CC2)nc1]

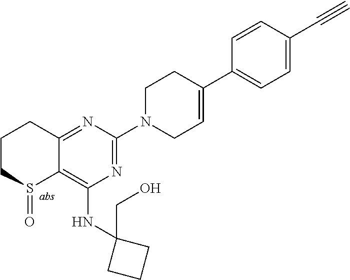 [SMILES: C#Cc1ccc(C2=CCN(c3nc4c(c(NC5(CO)CCC5)n3)[S@@](=O)CCC4)CC2)cc1]

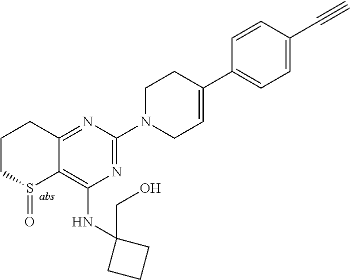 [SMILES: C#Cc1ccc(C2=CCN(c3nc4c(c(NC5(CO)CCC5)n3)[S@](=O)CCC4)CC2)cc1]

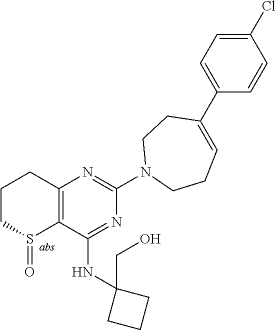 [SMILES: O=[S@@]1CCCc2nc(N3CCC=C(c4ccc(Cl)cc4)CC3)nc(NC3(CO)CCC3)c21]

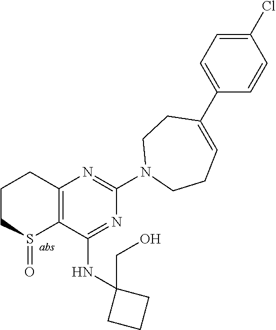 [SMILES: O=[S@]1CCCc2nc(N3CCC=C(c4ccc(Cl)cc4)CC3)nc(NC3(CO)CCC3)c21]

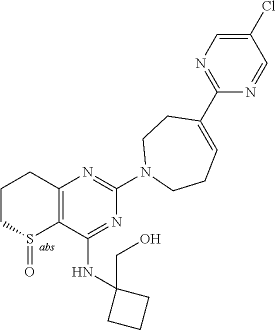 [SMILES: O=[S@@]1CCCc2nc(N3CCC=C(c4ncc(Cl)cn4)CC3)nc(NC3(CO)CCC3)c21]

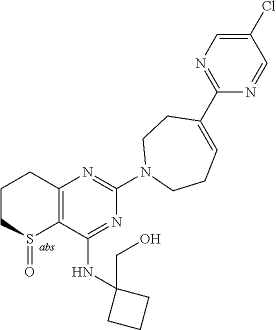 [SMILES: O=[S@]1CCCc2nc(N3CCC=C(c4ncc(Cl)cn4)CC3)nc(NC3(CO)CCC3)c21]

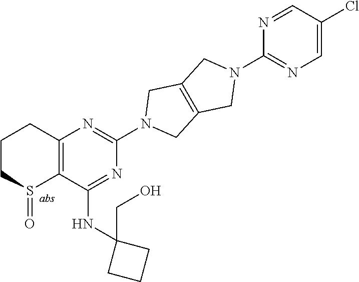 [SMILES: O=[S@]1CCCc2nc(N3CC4=C(CN(c5ncc(Cl)cn5)C4)C3)nc(NC3(CO)CCC3)c21]

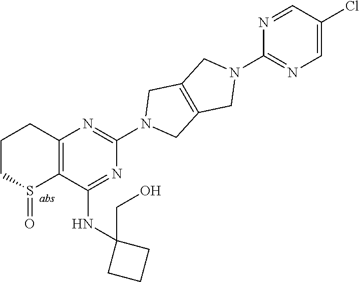 [SMILES: O=[S@@]1CCCc2nc(N3CC4=C(CN(c5ncc(Cl)cn5)C4)C3)nc(NC3(CO)CCC3)c21]

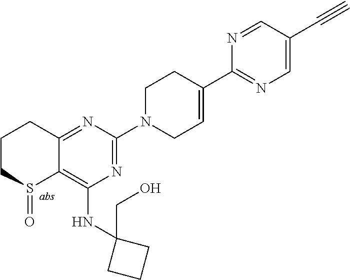 [SMILES: C#Cc1cnc(C2=CCN(c3nc4c(c(NC5(CO)CCC5)n3)[S@@](=O)CCC4)CC2)nc1]

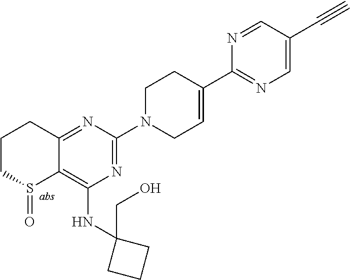 [SMILES: C#Cc1cnc(C2=CCN(c3nc4c(c(NC5(CO)CCC5)n3)[S@](=O)CCC4)CC2)nc1]

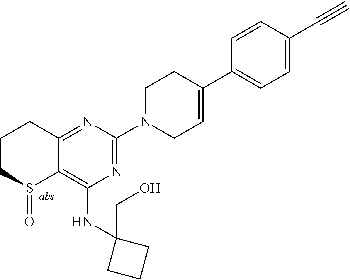 [SMILES: C#Cc1ccc(C2=CCN(c3nc4c(c(NC5(CO)CCC5)n3)[S@@](=O)CCC4)CC2)cc1]

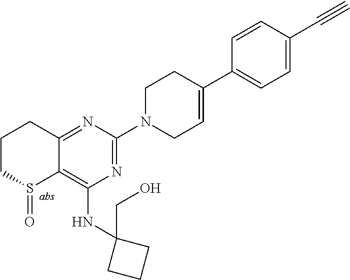 [SMILES: C#Cc1ccc(C2=CCN(c3nc4c(c(NC5(CO)CCC5)n3)[S@](=O)CCC4)CC2)cc1]

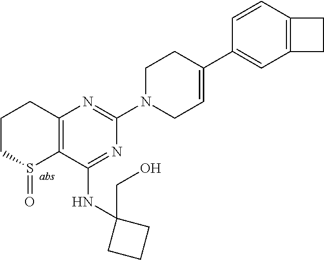 [SMILES: O=[S@@]1CCCc2nc(N3CC=C(c4ccc5c(c4)CC5)CC3)nc(NC3(CO)CCC3)c21]

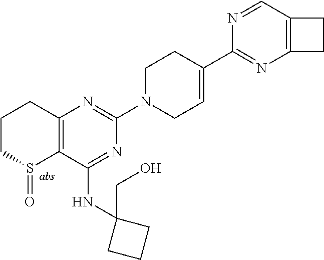 [SMILES: O=[S@@]1CCCc2nc(N3CC=C(c4ncc5c(n4)CC5)CC3)nc(NC3(CO)CCC3)c21]

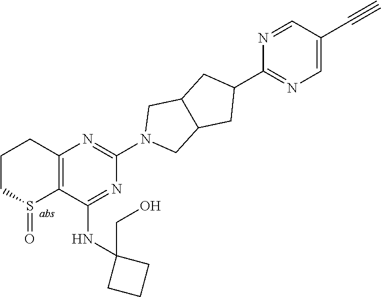 [SMILES: C#Cc1cnc(C2CC3CN(c4nc5c(c(NC6(CO)CCC6)n4)[S@](=O)CCC5)CC3C2)nc1]

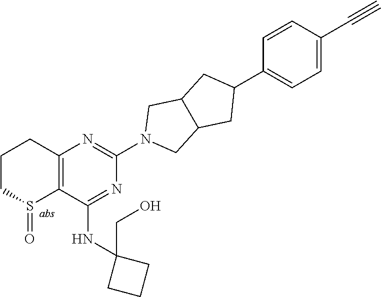 [SMILES: C#Cc1ccc(C2CC3CN(c4nc5c(c(NC6(CO)CCC6)n4)[S@](=O)CCC5)CC3C2)cc1]

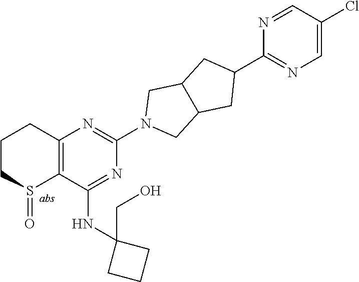 [SMILES: O=[S@]1CCCc2nc(N3CC4CC(c5ncc(Cl)cn5)CC4C3)nc(NC3(CO)CCC3)c21]

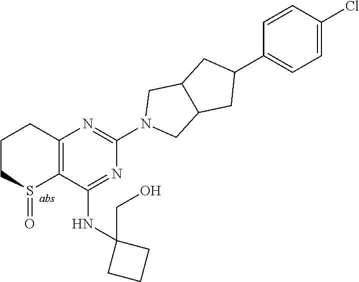 [SMILES: O=[S@]1CCCc2nc(N3CC4CC(c5ccc(Cl)cc5)CC4C3)nc(NC3(CO)CCC3)c21]

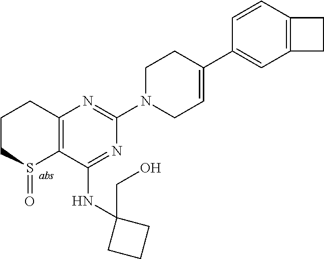 [SMILES: O=[S@]1CCCc2nc(N3CC=C(c4ccc5c(c4)CC5)CC3)nc(NC3(CO)CCC3)c21]

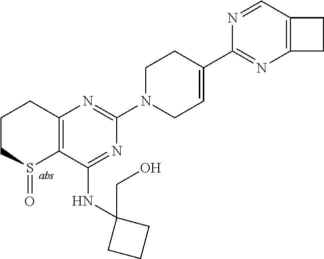 [SMILES: O=[S@]1CCCc2nc(N3CC=C(c4ncc5c(n4)CC5)CC3)nc(NC3(CO)CCC3)c21]

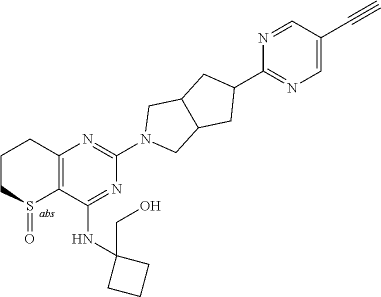 [SMILES: C#Cc1cnc(C2CC3CN(c4nc5c(c(NC6(CO)CCC6)n4)[S@@](=O)CCC5)CC3C2)nc1]

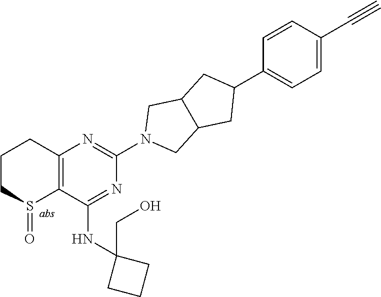 [SMILES: C#Cc1ccc(C2CC3CN(c4nc5c(c(NC6(CO)CCC6)n4)[S@@](=O)CCC5)CC3C2)cc1]

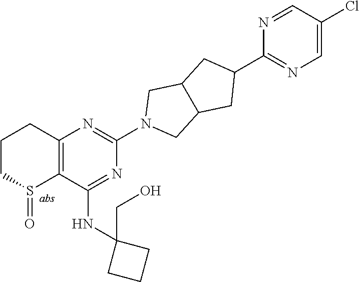 [SMILES: O=[S@@]1CCCc2nc(N3CC4CC(c5ncc(Cl)cn5)CC4C3)nc(NC3(CO)CCC3)c21]

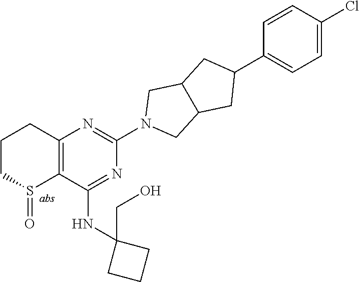 [SMILES: O=[S@@]1CCCc2nc(N3CC4CC(c5ccc(Cl)cc5)CC4C3)nc(NC3(CO)CCC3)c21]

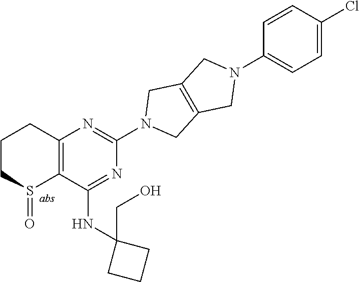 [SMILES: O=[S@]1CCCc2nc(N3CC4=C(CN(c5ccc(Cl)cc5)C4)C3)nc(NC3(CO)CCC3)c21]

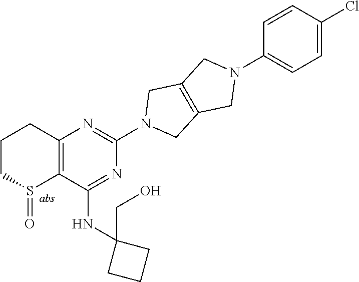 [SMILES: O=[S@@]1CCCc2nc(N3CC4=C(CN(c5ccc(Cl)cc5)C4)C3)nc(NC3(CO)CCC3)c21]

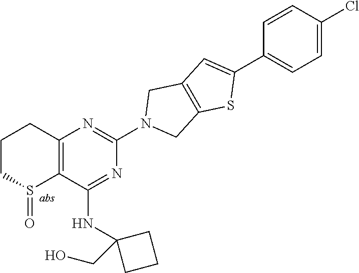 [SMILES: O=[S@@]1CCCc2nc(N3Cc4cc(-c5ccc(Cl)cc5)sc4C3)nc(NC3(CO)CCC3)c21]

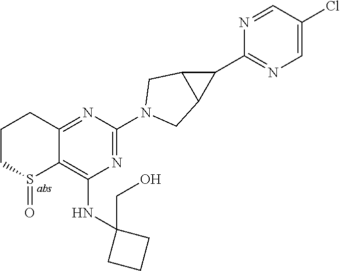 [SMILES: O=[S@@]1CCCc2nc(N3CC4C(C3)C4c3ncc(Cl)cn3)nc(NC3(CO)CCC3)c21]

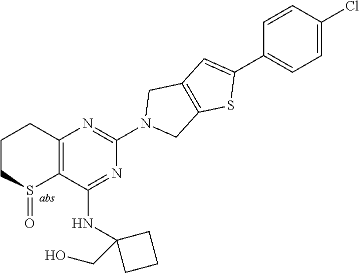 [SMILES: O=[S@]1CCCc2nc(N3Cc4cc(-c5ccc(Cl)cc5)sc4C3)nc(NC3(CO)CCC3)c21]

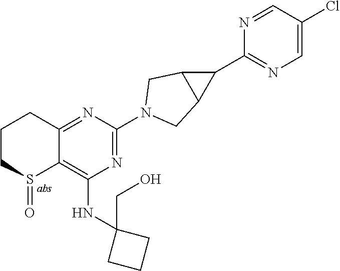 [SMILES: O=[S@]1CCCc2nc(N3CC4C(C3)C4c3ncc(Cl)cn3)nc(NC3(CO)CCC3)c21]

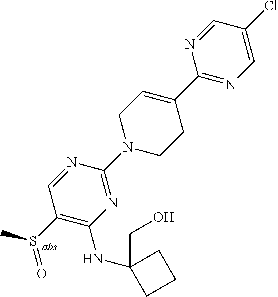 [SMILES: C[S@](=O)c1cnc(N2CC=C(c3ncc(Cl)cn3)CC2)nc1NC1(CO)CCC1]

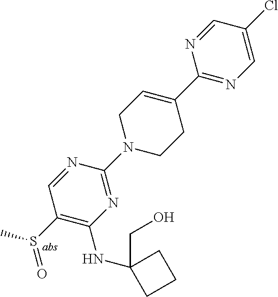 [SMILES: C[S@@](=O)c1cnc(N2CC=C(c3ncc(Cl)cn3)CC2)nc1NC1(CO)CCC1]

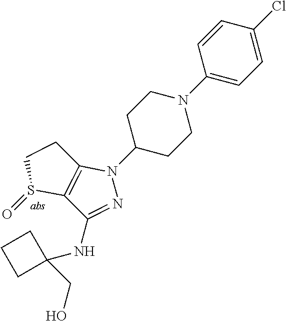 [SMILES: O=[S@@]1CCc2c1c(NC1(CO)CCC1)nn2C1CCN(c2ccc(Cl)cc2)CC1]

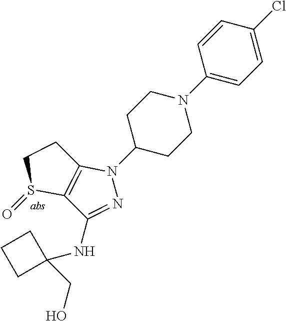 [SMILES: O=[S@]1CCc2c1c(NC1(CO)CCC1)nn2C1CCN(c2ccc(Cl)cc2)CC1]

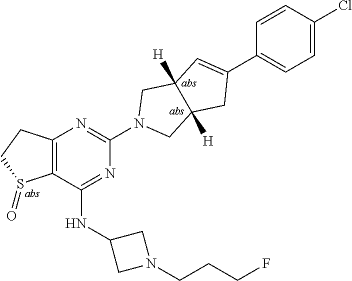 [SMILES: O=[S@@]1CCc2nc(N3C[C@H]4CC(c5ccc(Cl)cc5)=C[C@H]4C3)nc(NC3CN(CCCF)C3)c21]

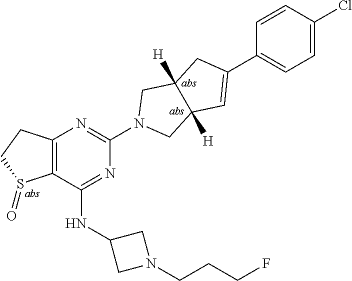 [SMILES: O=[S@@]1CCc2nc(N3C[C@@H]4CC(c5ccc(Cl)cc5)=C[C@@H]4C3)nc(NC3CN(CCCF)C3)c21]

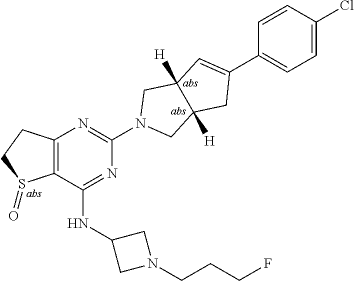 [SMILES: O=[S@]1CCc2nc(N3C[C@H]4CC(c5ccc(Cl)cc5)=C[C@H]4C3)nc(NC3CN(CCCF)C3)c21]

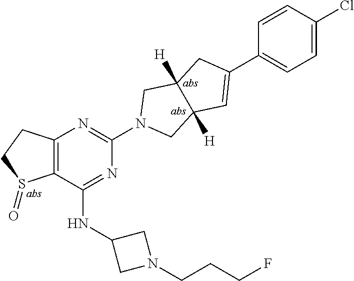 [SMILES: O=[S@]1CCc2nc(N3C[C@@H]4CC(c5ccc(Cl)cc5)=C[C@@H]4C3)nc(NC3CN(CCCF)C3)c21]

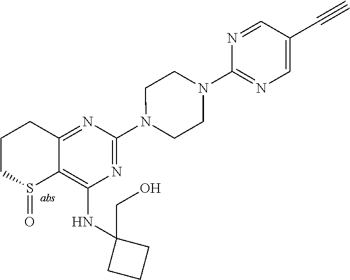 [SMILES: C#Cc1cnc(N2CCN(c3nc4c(c(NC5(CO)CCC5)n3)[S@](=O)CCC4)CC2)nc1]

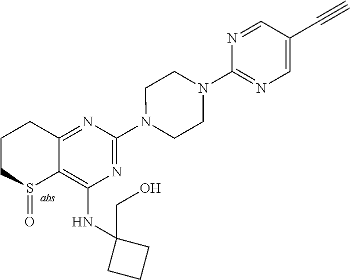 [SMILES: C#Cc1cnc(N2CCN(c3nc4c(c(NC5(CO)CCC5)n3)[S@@](=O)CCC4)CC2)nc1]

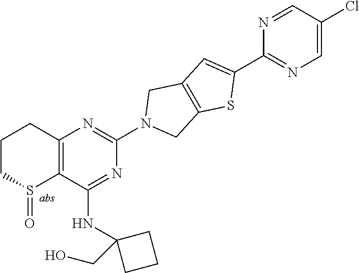 [SMILES: O=[S@@]1CCCc2nc(N3Cc4cc(-c5ncc(Cl)cn5)sc4C3)nc(NC3(CO)CCC3)c21]

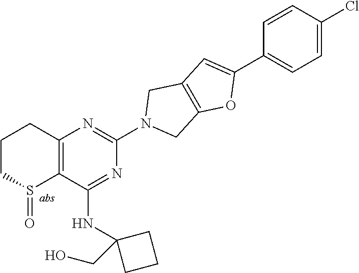 [SMILES: O=[S@@]1CCCc2nc(N3Cc4cc(-c5ccc(Cl)cc5)oc4C3)nc(NC3(CO)CCC3)c21]

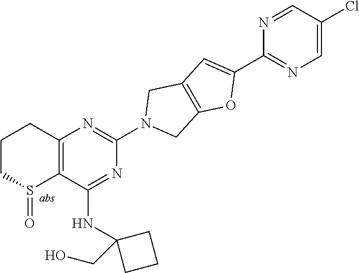 [SMILES: O=[S@@]1CCCc2nc(N3Cc4cc(-c5ncc(Cl)cn5)oc4C3)nc(NC3(CO)CCC3)c21]

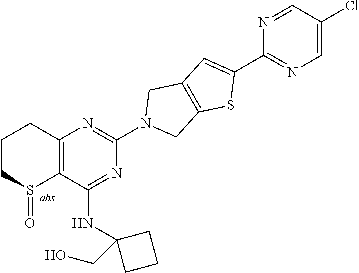 [SMILES: O=[S@]1CCCc2nc(N3Cc4cc(-c5ncc(Cl)cn5)sc4C3)nc(NC3(CO)CCC3)c21]

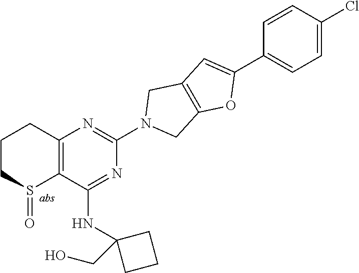 [SMILES: O=[S@]1CCCc2nc(N3Cc4cc(-c5ccc(Cl)cc5)oc4C3)nc(NC3(CO)CCC3)c21]

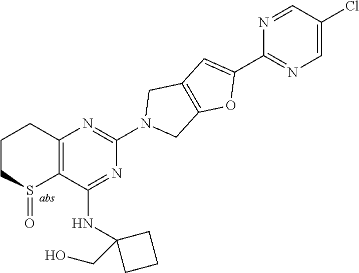 [SMILES: O=[S@]1CCCc2nc(N3Cc4cc(-c5ncc(Cl)cn5)oc4C3)nc(NC3(CO)CCC3)c21]

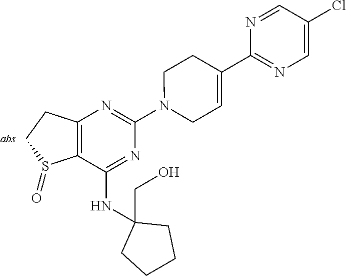

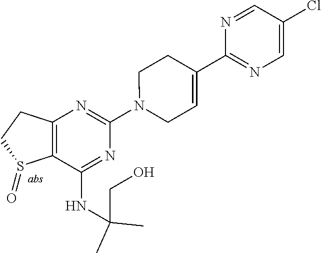 [SMILES: CC(C)(CO)Nc1nc(N2CC=C(c3ncc(Cl)cn3)CC2)nc2c1[S@](=O)CC2]

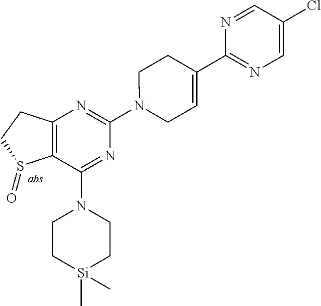 [SMILES: C[Si]1(C)CCN(c2nc(N3CC=C(c4ncc(Cl)cn4)CC3)nc3c2[S@](=O)CC3)CC1]

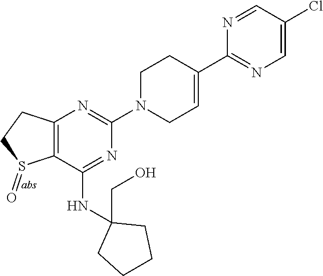 [SMILES: O=[S@]1CCc2nc(N3CC=C(c4ncc(Cl)cn4)CC3)nc(NC3(CO)CCCC3)c21]

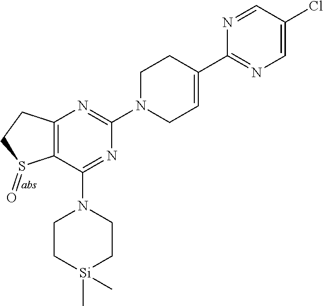 [SMILES: C[Si]1(C)CCN(c2nc(N3CC=C(c4ncc(Cl)cn4)CC3)nc3c2[S@@](=O)CC3)CC1]

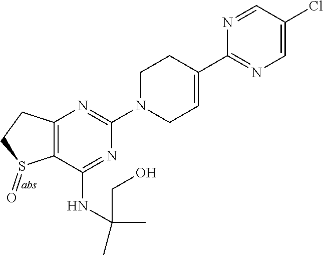 [SMILES: CC(C)(CO)Nc1nc(N2CC=C(c3ncc(Cl)cn3)CC2)nc2c1[S@@](=O)CC2]

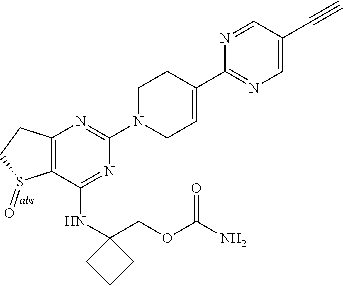 [SMILES: C#Cc1cnc(C2=CCN(c3nc4c(c(NC5(COC(N)=O)CCC5)n3)[S@](=O)CC4)CC2)nc1]

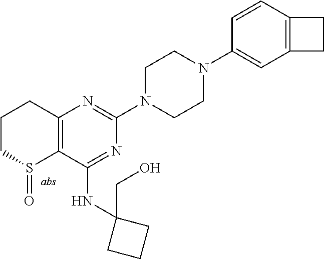 [SMILES: O=[S@@]1CCCc2nc(N3CCN(c4ccc5c(c4)CC5)CC3)nc(NC3(CO)CCC3)c21]

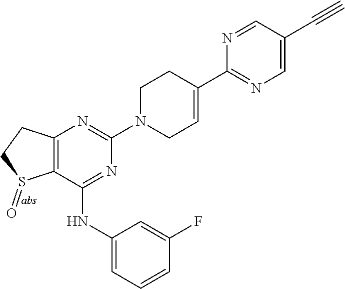 [SMILES: C#Cc1cnc(C2=CCN(c3nc4c(c(Nc5cccc(F)c5)n3)[S@@](=O)CC4)CC2)nc1]

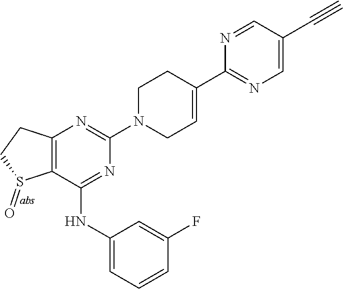 [SMILES: C#Cc1cnc(C2=CCN(c3nc4c(c(Nc5cccc(F)c5)n3)[S@](=O)CC4)CC2)nc1]

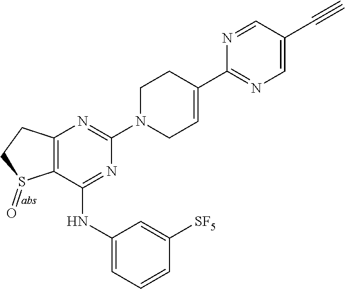

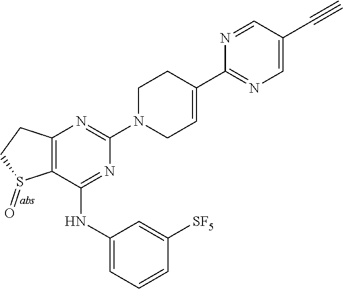

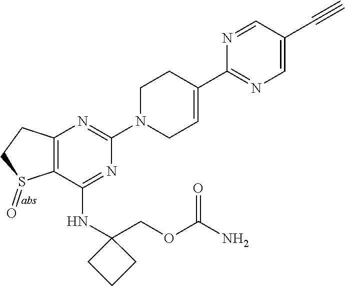 [SMILES: C#Cc1cnc(C2=CCN(c3nc4c(c(NC5(COC(N)=O)CCC5)n3)[S@@](=O)CC4)CC2)nc1]

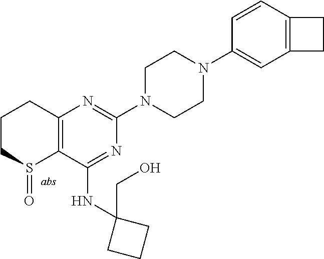 [SMILES: O=[S@]1CCCc2nc(N3CCN(c4ccc5c(c4)CC5)CC3)nc(NC3(CO)CCC3)c21]

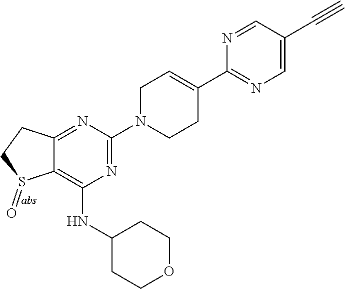 [SMILES: C#Cc1cnc(C2=CCN(c3nc4c(c(NC5CCOCC5)n3)[S@@](=O)CC4)CC2)nc1]

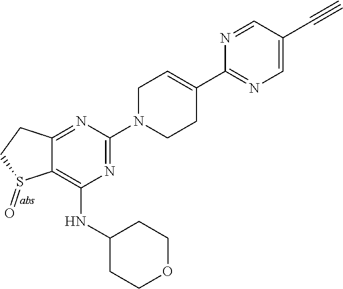 [SMILES: C#Cc1cnc(C2=CCN(c3nc4c(c(NC5CCOCC5)n3)[S@](=O)CC4)CC2)nc1]

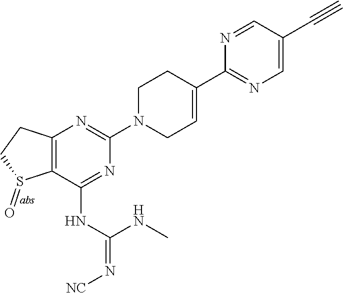 [SMILES: C#Cc1cnc(C2=CCN(c3nc4c(c(N/C(=N\C#N)NC)n3)[S@](=O)CC4)CC2)nc1]

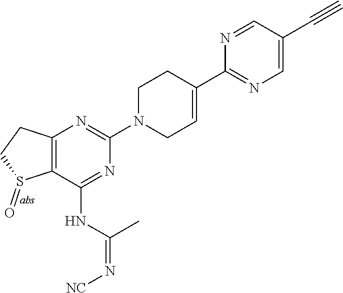 [SMILES: C#Cc1cnc(C2=CCN(c3nc4c(c(N/C(C)=N\C#N)n3)[S@](=O)CC4)CC2)nc1]

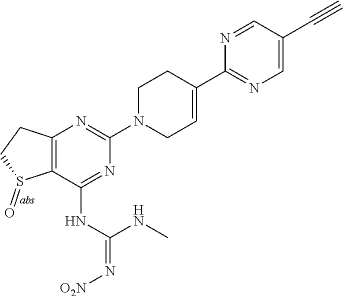 [SMILES: C#Cc1cnc(C2=CCN(c3nc4c(c(N/C(=N\[N+](=O)[O-])NC)n3)[S@](=O)CC4)CC2)nc1]

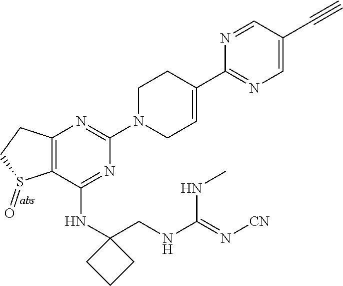 [SMILES: C#Cc1cnc(C2=CCN(c3nc4c(c(NC5(CN/C(=N/C#N)NC)CCC5)n3)[S@](=O)CC4)CC2)nc1]

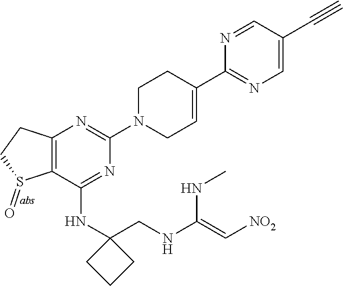 [SMILES: C#Cc1cnc(C2=CCN(c3nc4c(c(NC5(CN/C(=C/[N+](=O)[O-])NC)CCC5)n3)[S@](=O)CC4)CC2)nc1]

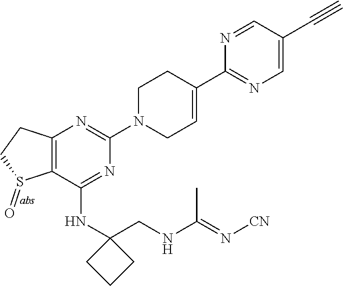 [SMILES: C#Cc1cnc(C2=CCN(c3nc4c(c(NC5(CN/C(C)=N/C#N)CCC5)n3)[S@](=O)CC4)CC2)nc1]

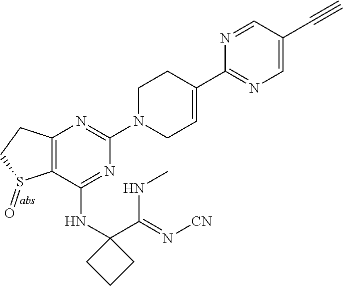 [SMILES: C#Cc1cnc(C2=CCN(c3nc4c(c(NC5(/C(=N/C#N)NC)CCC5)n3)[S@](=O)CC4)CC2)nc1]

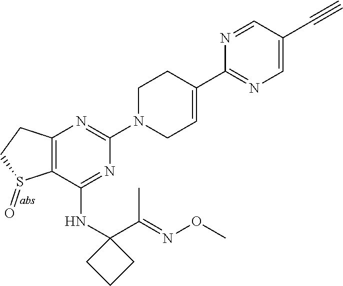 [SMILES: C#Cc1cnc(C2=CCN(c3nc4c(c(NC5(/C(C)=N/OC)CCC5)n3)[S@](=O)CC4)CC2)nc1]

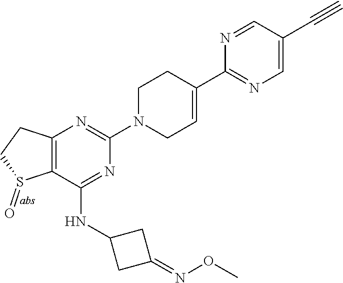 [SMILES: C#Cc1cnc(C2=CCN(c3nc4c(c(NC5CC(=NOC)C5)n3)[S@](=O)CC4)CC2)nc1]

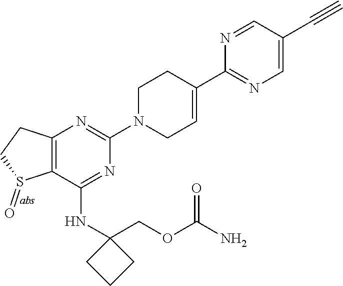 [SMILES: C#Cc1cnc(C2=CCN(c3nc4c(c(NC5(COC(N)=O)CCC5)n3)[S@](=O)CC4)CC2)nc1]

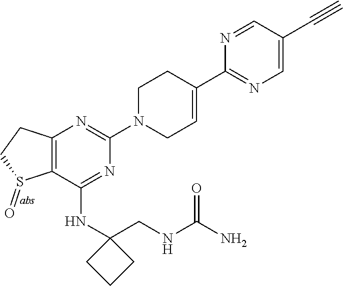 [SMILES: C#Cc1cnc(C2=CCN(c3nc4c(c(NC5(CNC(N)=O)CCC5)n3)[S@](=O)CC4)CC2)nc1]

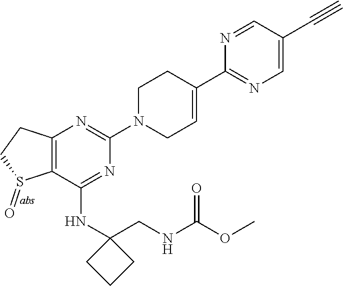 [SMILES: C#Cc1cnc(C2=CCN(c3nc4c(c(NC5(CNC(=O)OC)CCC5)n3)[S@](=O)CC4)CC2)nc1]

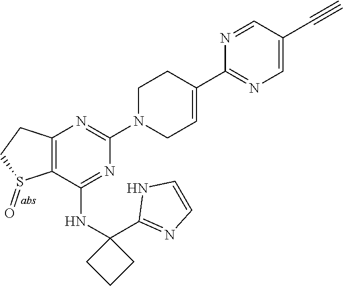 [SMILES: C#Cc1cnc(C2=CCN(c3nc4c(c(NC5(c6ncc[nH]6)CCC5)n3)[S@](=O)CC4)CC2)nc1]

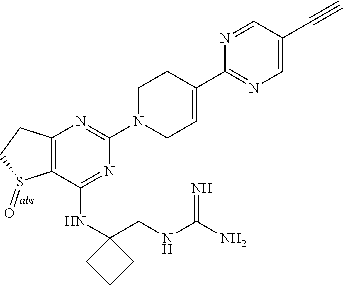 [SMILES: C#Cc1cnc(C2=CCN(c3nc4c(c(NC5(CNC(=N)N)CCC5)n3)[S@](=O)CC4)CC2)nc1]

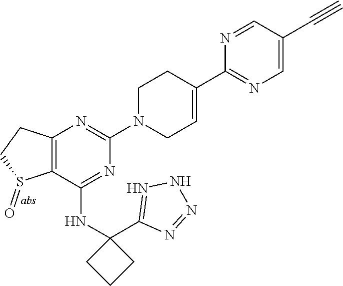 [SMILES: C#Cc1cnc(C2=CCN(c3nc4c(c(NC5(c6nn[nH]n6)CCC5)n3)[S@](=O)CC4)CC2)nc1]

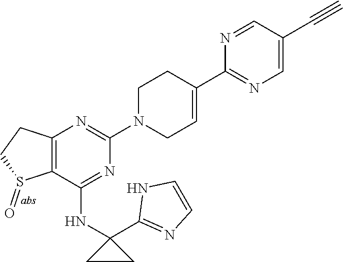 [SMILES: C#Cc1cnc(C2=CCN(c3nc4c(c(NC5(c6ncc[nH]6)CC5)n3)[S@](=O)CC4)CC2)nc1]

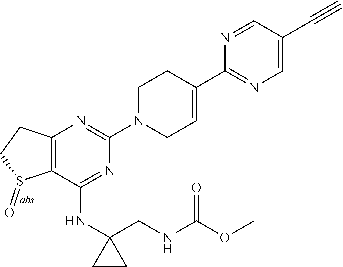 [SMILES: C#Cc1cnc(C2=CCN(c3nc4c(c(NC5(CNC(=O)OC)CC5)n3)[S@](=O)CC4)CC2)nc1]

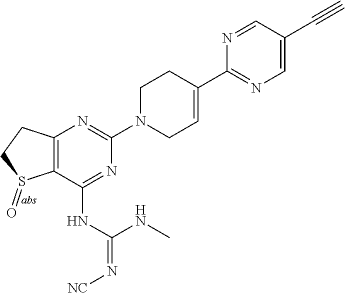 [SMILES: C#Cc1cnc(C2=CCN(c3nc4c(c(N/C(=N\C#N)NC)n3)[S@@](=O)CC4)CC2)nc1]

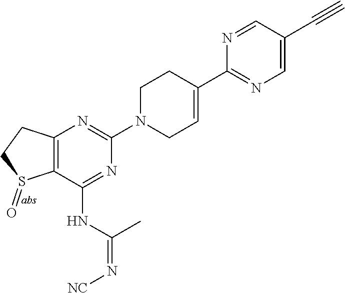 [SMILES: C#Cc1cnc(C2=CCN(c3nc4c(c(N/C(C)=N\C#N)n3)[S@@](=O)CC4)CC2)nc1]

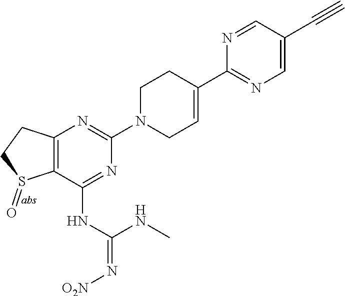 [SMILES: C#Cc1cnc(C2=CCN(c3nc4c(c(N/C(=N\[N+](=O)[O-])NC)n3)[S@@](=O)CC4)CC2)nc1]

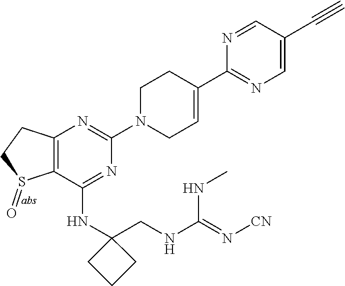 [SMILES: C#Cc1cnc(C2=CCN(c3nc4c(c(NC5(CN/C(=N/C#N)NC)CCC5)n3)[S@@](=O)CC4)CC2)nc1]

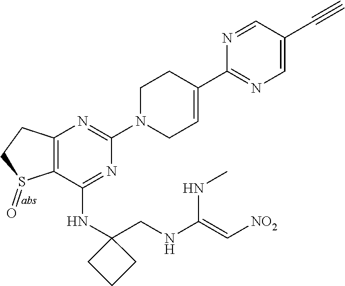 [SMILES: C#Cc1cnc(C2=CCN(c3nc4c(c(NC5(CN/C(=C/[N+](=O)[O-])NC)CCC5)n3)[S@@](=O)CC4)CC2)nc1]

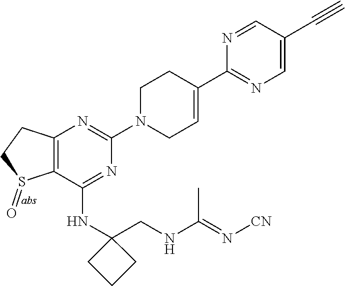 [SMILES: C#Cc1cnc(C2=CCN(c3nc4c(c(NC5(CN/C(C)=N/C#N)CCC5)n3)[S@@](=O)CC4)CC2)nc1]

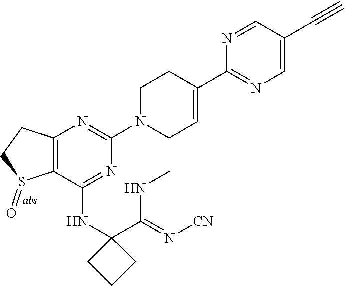 [SMILES: C#Cc1cnc(C2=CCN(c3nc4c(c(NC5(/C(=N/C#N)NC)CCC5)n3)[S@@](=O)CC4)CC2)nc1]

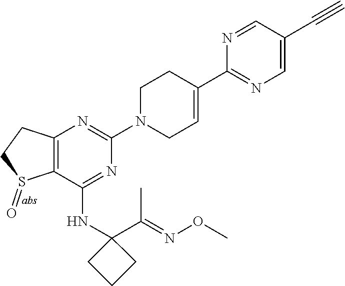 [SMILES: C#Cc1cnc(C2=CCN(c3nc4c(c(NC5(/C(C)=N/OC)CCC5)n3)[S@@](=O)CC4)CC2)nc1]

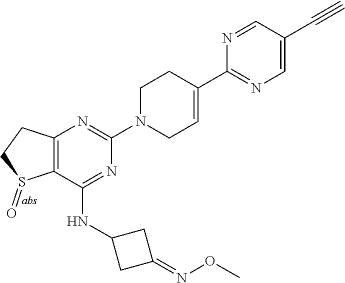 [SMILES: C#Cc1cnc(C2=CCN(c3nc4c(c(NC5CC(=NOC)C5)n3)[S@@](=O)CC4)CC2)nc1]

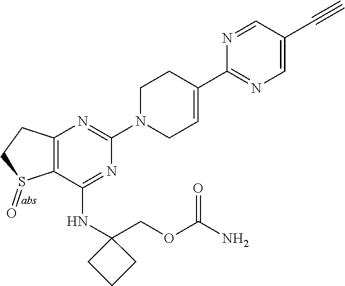 [SMILES: C#Cc1cnc(C2=CCN(c3nc4c(c(NC5(COC(N)=O)CCC5)n3)[S@@](=O)CC4)CC2)nc1]

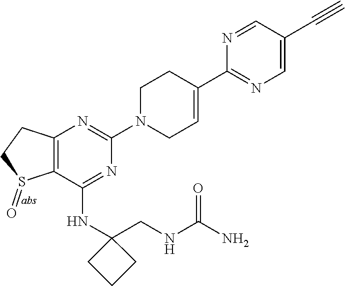 [SMILES: C#Cc1cnc(C2=CCN(c3nc4c(c(NC5(CNC(N)=O)CCC5)n3)[S@@](=O)CC4)CC2)nc1]

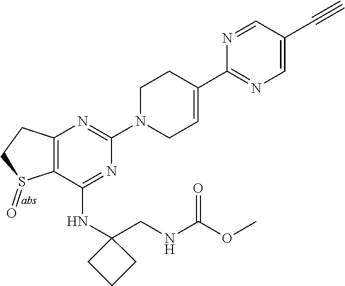 [SMILES: C#Cc1cnc(C2=CCN(c3nc4c(c(NC5(CNC(=O)OC)CCC5)n3)[S@@](=O)CC4)CC2)nc1]

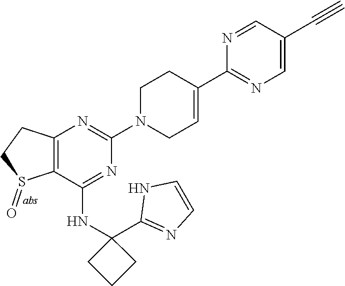 [SMILES: C#Cc1cnc(C2=CCN(c3nc4c(c(NC5(c6ncc[nH]6)CCC5)n3)[S@@](=O)CC4)CC2)nc1]

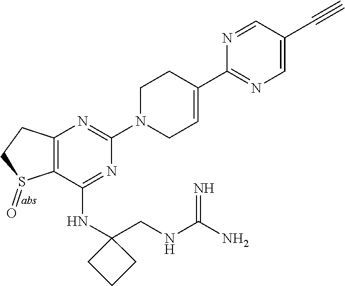 [SMILES: C#Cc1cnc(C2=CCN(c3nc4c(c(NC5(CNC(=N)N)CCC5)n3)[S@@](=O)CC4)CC2)nc1]

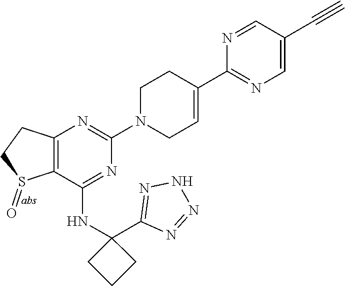 [SMILES: C#Cc1cnc(C2=CCN(c3nc4c(c(NC5(c6nn[nH]n6)CCC5)n3)[S@@](=O)CC4)CC2)nc1]

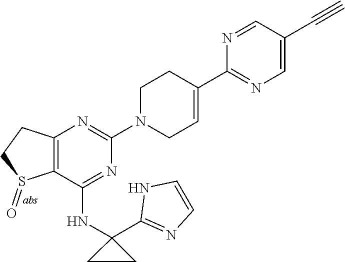 [SMILES: C#Cc1cnc(C2=CCN(c3nc4c(c(NC5(c6ncc[nH]6)CC5)n3)[S@@](=O)CC4)CC2)nc1]

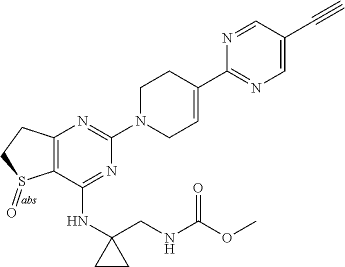 [SMILES: C#Cc1cnc(C2=CCN(c3nc4c(c(NC5(CNC(=O)OC)CC5)n3)[S@@](=O)CC4)CC2)nc1]

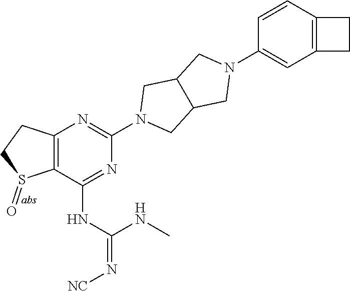 [SMILES: CN/C(=N/C#N)Nc1nc(N2CC3CN(c4ccc5c(c4)CC5)CC3C2)nc2c1[S@@](=O)CC2]

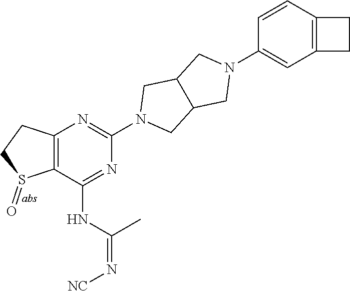 [SMILES: C/C(=N/C#N)Nc1nc(N2CC3CN(c4ccc5c(c4)CC5)CC3C2)nc2c1[S@@](=O)CC2]

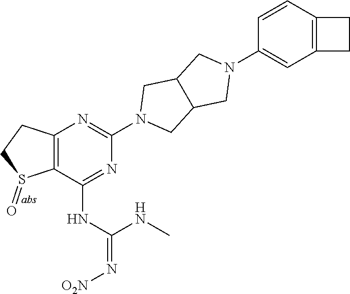 [SMILES: CN/C(=N/[N+](=O)[O-])Nc1nc(N2CC3CN(c4ccc5c(c4)CC5)CC3C2)nc2c1[S@@](=O)CC2]

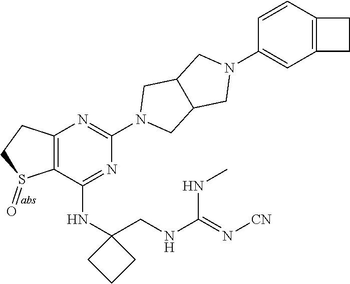 [SMILES: CN/C(=N\C#N)NCC1(Nc2nc(N3CC4CN(c5ccc6c(c5)CC6)CC4C3)nc3c2[S@@](=O)CC3)CCC1]

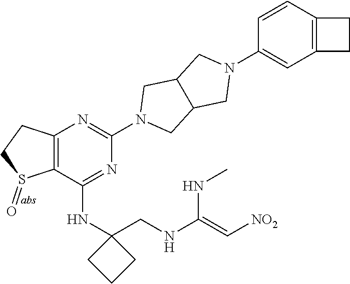 [SMILES: CN/C(=C\[N+](=O)[O-])NCC1(Nc2nc(N3CC4CN(c5ccc6c(c5)CC6)CC4C3)nc3c2[S@@](=O)CC3)CCC1]

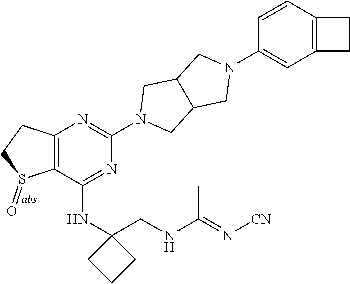 [SMILES: C/C(=N\C#N)NCC1(Nc2nc(N3CC4CN(c5ccc6c(c5)CC6)CC4C3)nc3c2[S@@](=O)CC3)CCC1]

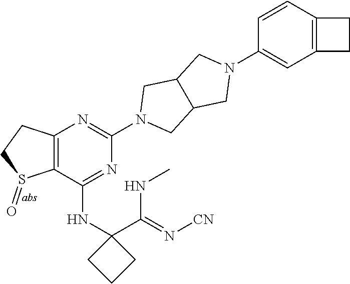 [SMILES: CN/C(=N\C#N)C1(Nc2nc(N3CC4CN(c5ccc6c(c5)CC6)CC4C3)nc3c2[S@@](=O)CC3)CCC1]

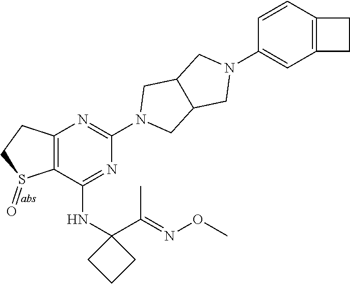 [SMILES: CO/N=C(\C)C1(Nc2nc(N3CC4CN(c5ccc6c(c5)CC6)CC4C3)nc3c2[S@@](=O)CC3)CCC1]

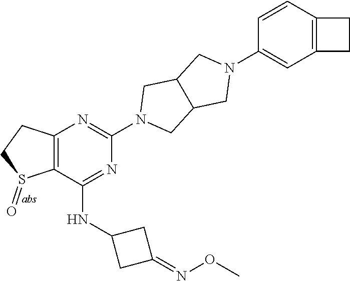 [SMILES: CON=C1CC(Nc2nc(N3CC4CN(c5ccc6c(c5)CC6)CC4C3)nc3c2[S@@](=O)CC3)C1]

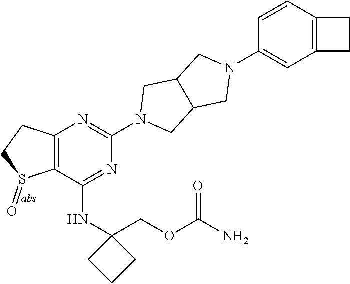 [SMILES: NC(=O)OCC1(Nc2nc(N3CC4CN(c5ccc6c(c5)CC6)CC4C3)nc3c2[S@@](=O)CC3)CCC1]

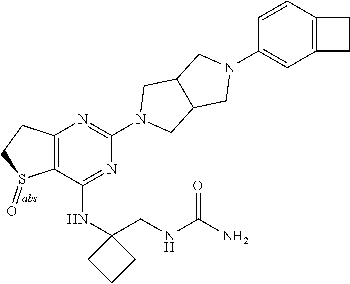 [SMILES: NC(=O)NCC1(Nc2nc(N3CC4CN(c5ccc6c(c5)CC6)CC4C3)nc3c2[S@@](=O)CC3)CCC1]

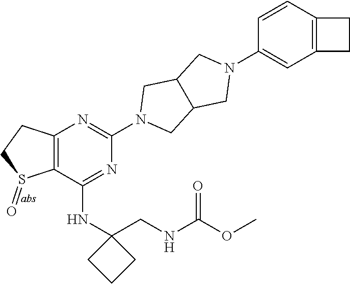 [SMILES: COC(=O)NCC1(Nc2nc(N3CC4CN(c5ccc6c(c5)CC6)CC4C3)nc3c2[S@@](=O)CC3)CCC1]

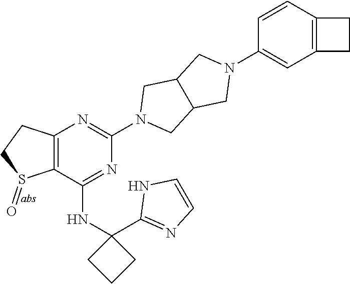 [SMILES: O=[S@]1CCc2nc(N3CC4CN(c5ccc6c(c5)CC6)CC4C3)nc(NC3(c4ncc[nH]4)CCC3)c21]

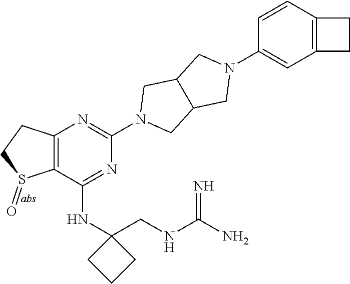 [SMILES: N=C(N)NCC1(Nc2nc(N3CC4CN(c5ccc6c(c5)CC6)CC4C3)nc3c2[S@@](=O)CC3)CCC1]

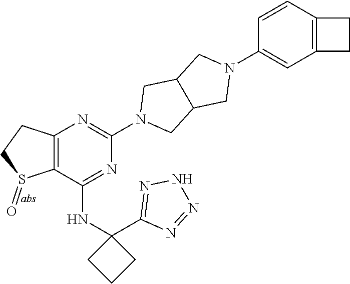 [SMILES: O=[S@]1CCc2nc(N3CC4CN(c5ccc6c(c5)CC6)CC4C3)nc(NC3(c4nn[nH]n4)CCC3)c21]

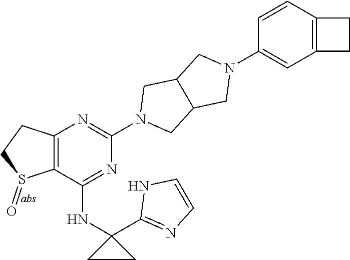 [SMILES: O=[S@]1CCc2nc(N3CC4CN(c5ccc6c(c5)CC6)CC4C3)nc(NC3(c4ncc[nH]4)CC3)c21]

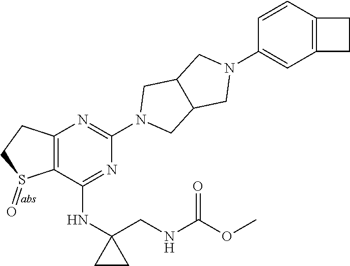 [SMILES: COC(=O)NCC1(Nc2nc(N3CC4CN(c5ccc6c(c5)CC6)CC4C3)nc3c2[S@@](=O)CC3)CC1]

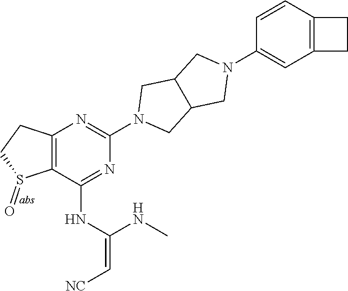 [SMILES: CN/C(=C/C#N)Nc1nc(N2CC3CN(c4ccc5c(c4)CC5)CC3C2)nc2c1[S@](=O)CC2]

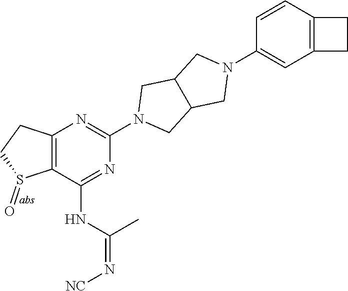 [SMILES: C/C(=N/C#N)Nc1nc(N2CC3CN(c4ccc5c(c4)CC5)CC3C2)nc2c1[S@](=O)CC2]

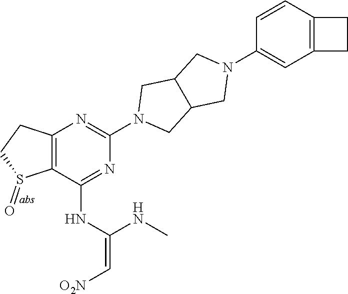 [SMILES: CN/C(=C/[N+](=O)[O-])Nc1nc(N2CC3CN(c4ccc5c(c4)CC5)CC3C2)nc2c1[S@](=O)CC2]

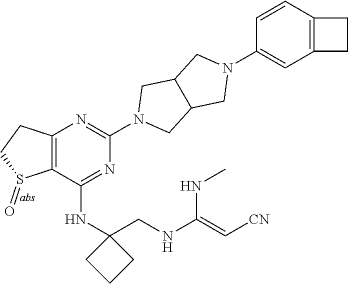 [SMILES: CN/C(=C\C#N)NCC1(Nc2nc(N3CC4CN(c5ccc6c(c5)CC6)CC4C3)nc3c2[S@](=O)CC3)CCC1]

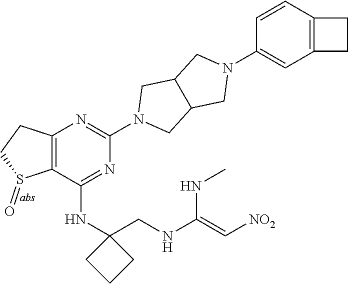 [SMILES: CN/C(=C\[N+](=O)[O-])NCC1(Nc2nc(N3CC4CN(c5ccc6c(c5)CC6)CC4C3)nc3c2[S@](=O)CC3)CCC1]

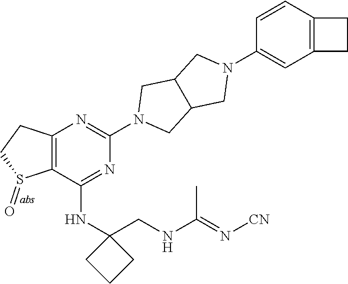 [SMILES: C/C(=N\C#N)NCC1(Nc2nc(N3CC4CN(c5ccc6c(c5)CC6)CC4C3)nc3c2[S@](=O)CC3)CCC1]

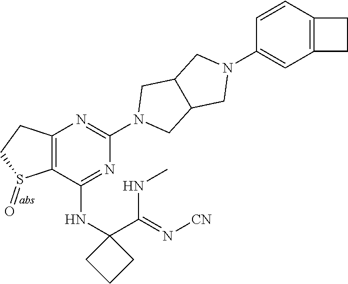 [SMILES: CN/C(=N\C#N)C1(Nc2nc(N3CC4CN(c5ccc6c(c5)CC6)CC4C3)nc3c2[S@](=O)CC3)CCC1]

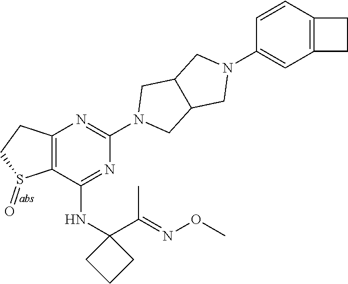 [SMILES: CO/N=C(\C)C1(Nc2nc(N3CC4CN(c5ccc6c(c5)CC6)CC4C3)nc3c2[S@](=O)CC3)CCC1]

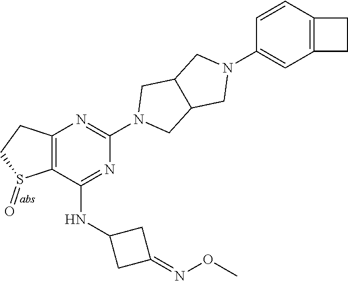 [SMILES: CON=C1CC(Nc2nc(N3CC4CN(c5ccc6c(c5)CC6)CC4C3)nc3c2[S@](=O)CC3)C1]

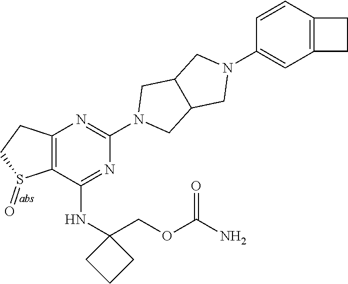 [SMILES: NC(=O)OCC1(Nc2nc(N3CC4CN(c5ccc6c(c5)CC6)CC4C3)nc3c2[S@](=O)CC3)CCC1]

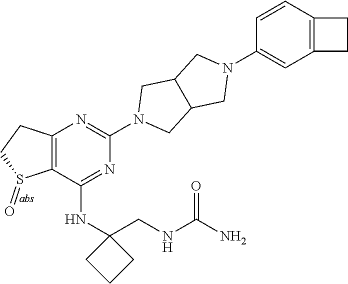 [SMILES: NC(=O)NCC1(Nc2nc(N3CC4CN(c5ccc6c(c5)CC6)CC4C3)nc3c2[S@](=O)CC3)CCC1]

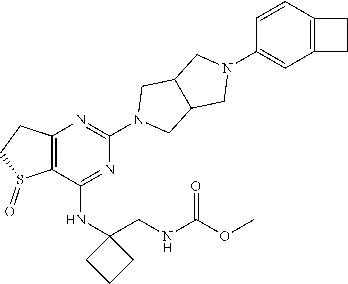 [SMILES: COC(=O)NCC1(Nc2nc(N3CC4CN(c5ccc6c(c5)CC6)CC4C3)nc3c2[S@](=O)CC3)CCC1]

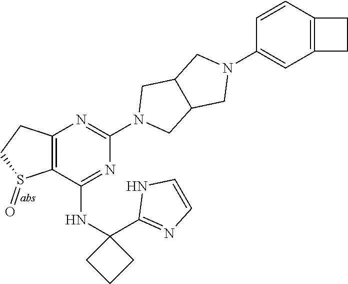 [SMILES: O=[S@@]1CCc2nc(N3CC4CN(c5ccc6c(c5)CC6)CC4C3)nc(NC3(c4ncc[nH]4)CCC3)c21]

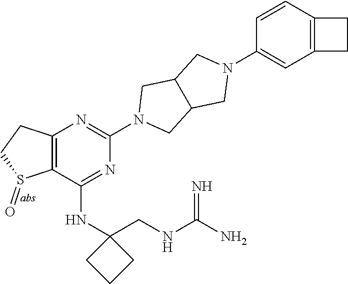 [SMILES: N=C(N)NCC1(Nc2nc(N3CC4CN(c5ccc6c(c5)CC6)CC4C3)nc3c2[S@](=O)CC3)CCC1]

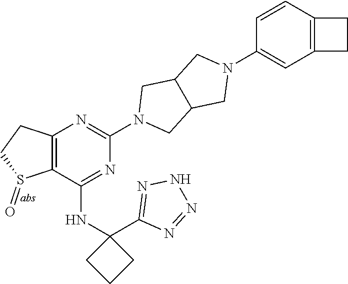 [SMILES: O=[S@@]1CCc2nc(N3CC4CN(c5ccc6c(c5)CC6)CC4C3)nc(NC3(c4nn[nH]n4)CCC3)c21]

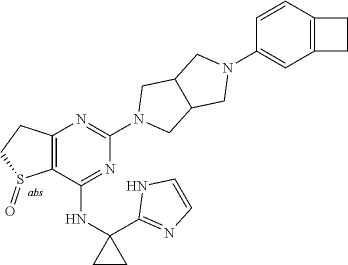 [SMILES: O=[S@@]1CCc2nc(N3CC4CN(c5ccc6c(c5)CC6)CC4C3)nc(NC3(c4ncc[nH]4)CC3)c21]

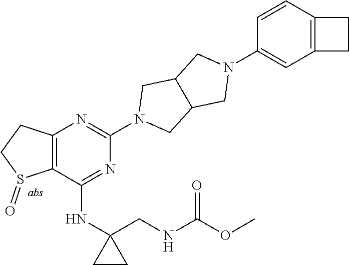 [SMILES: COC(=O)NCC1(Nc2nc(N3CC4CN(c5ccc6c(c5)CC6)CC4C3)nc3c2S(=O)CC3)CC1]

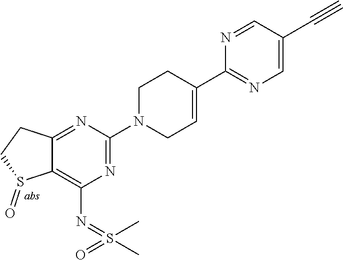 [SMILES: C#Cc1cnc(C2=CCN(c3nc4c(c(N=S(C)(C)=O)n3)[S@](=O)CC4)CC2)nc1]

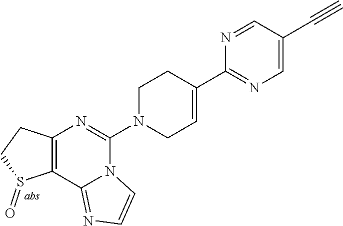 [SMILES: C#Cc1cnc(C2=CCN(c3nc4c(c5nccn35)[S@](=O)CC4)CC2)nc1]

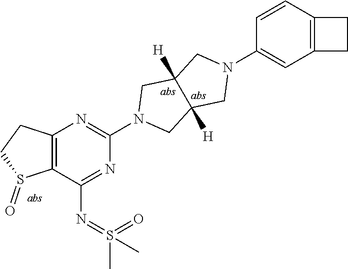 [SMILES: CS(C)(=O)=Nc1nc(N2C[C@H]3CN(c4ccc5c(c4)CC5)C[C@H]3C2)nc2c1[S@](=O)CC2]

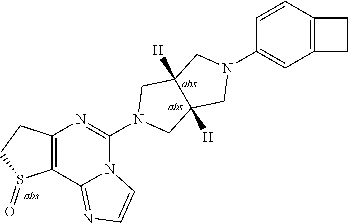 [SMILES: O=[S@@]1CCc2nc(N3C[C@H]4CN(c5ccc6c(c5)CC6)C[C@H]4C3)n3ccnc3c21]

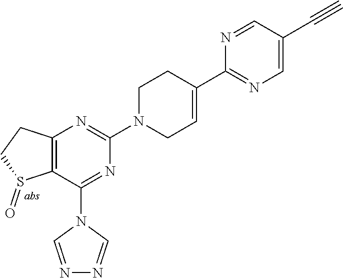 [SMILES: C#Cc1cnc(C2=CCN(c3nc4c(c(-n5cnnc5)n3)[S@](=O)CC4)CC2)nc1]

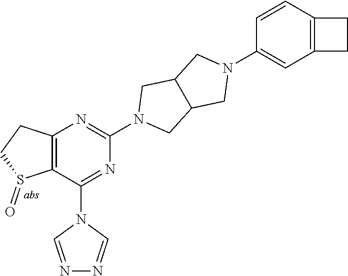 [SMILES: O=[S@@]1CCc2nc(N3CC4CN(c5ccc6c(c5)CC6)CC4C3)nc(-n3cnnc3)c21]

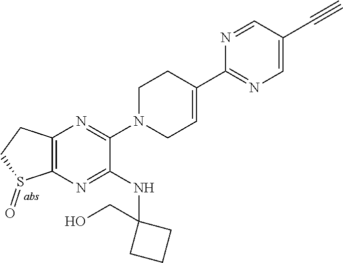 [SMILES: C#Cc1cnc(C2=CCN(c3nc4c(nc3NC3(CO)CCC3)[S@](=O)CC4)CC2)nc1]

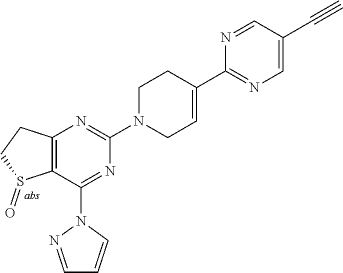 [SMILES: C#Cc1cnc(C2=CCN(c3nc4c(c(-n5cccn5)n3)[S@](=O)CC4)CC2)nc1]

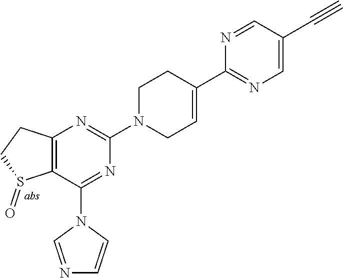 [SMILES: C#Cc1cnc(C2=CCN(c3nc4c(c(-n5ccnc5)n3)[S@](=O)CC4)CC2)nc1]

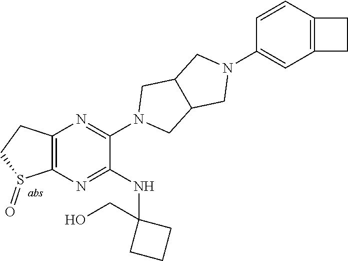 [SMILES: O=[S@@]1CCc2nc(N3CC4CN(c5ccc6c(c5)CC6)CC4C3)c(NC3(CO)CCC3)nc21]

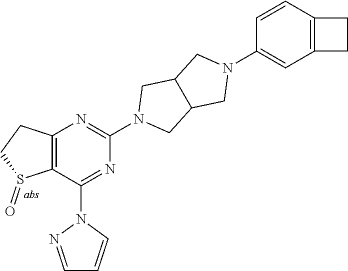 [SMILES: O=[S@@]1CCc2nc(N3CC4CN(c5ccc6c(c5)CC6)CC4C3)nc(-n3cccn3)c21]

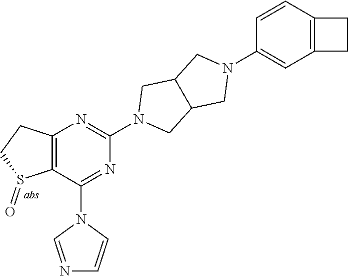 [SMILES: O=[S@@]1CCc2nc(N3CC4CN(c5ccc6c(c5)CC6)CC4C3)nc(-n3ccnc3)c21]

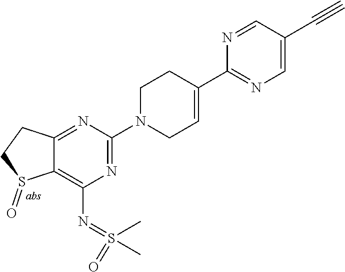 [SMILES: C#Cc1cnc(C2=CCN(c3nc4c(c(N=S(C)(C)=O)n3)[S@@](=O)CC4)CC2)nc1]

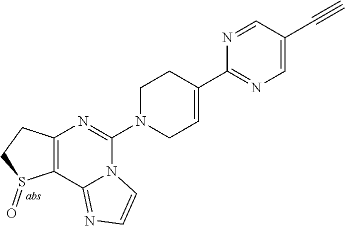 [SMILES: C#Cc1cnc(C2=CCN(c3nc4c(c5nccn35)[S@@](=O)CC4)CC2)nc1]

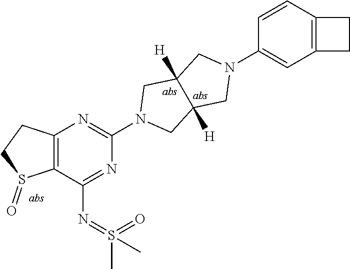 [SMILES: CS(C)(=O)=Nc1nc(N2C[C@H]3CN(c4ccc5c(c4)CC5)C[C@H]3C2)nc2c1[S@@](=O)CC2]

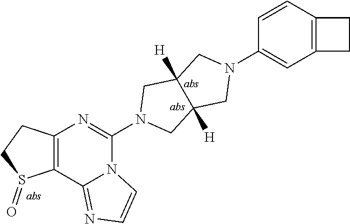 [SMILES: O=[S@]1CCc2nc(N3C[C@H]4CN(c5ccc6c(c5)CC6)C[C@H]4C3)n3ccnc3c21]

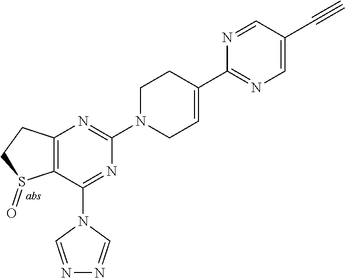 [SMILES: C#Cc1cnc(C2=CCN(c3nc4c(c(-n5cnnc5)n3)[S@@](=O)CC4)CC2)nc1]

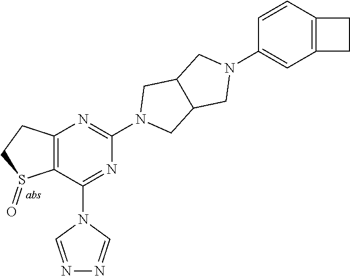 [SMILES: O=[S@]1CCc2nc(N3CC4CN(c5ccc6c(c5)CC6)CC4C3)nc(-n3cnnc3)c21]

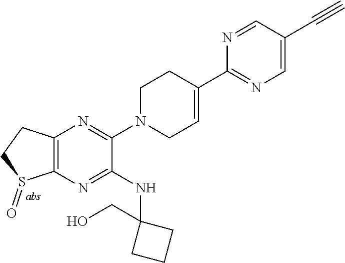 [SMILES: C#Cc1cnc(C2=CCN(c3nc4c(nc3NC3(CO)CCC3)[S@@](=O)CC4)CC2)nc1]

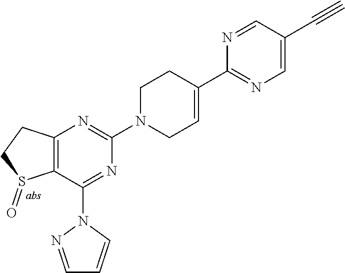 [SMILES: C#Cc1cnc(C2=CCN(c3nc4c(c(-n5cccn5)n3)[S@@](=O)CC4)CC2)nc1]

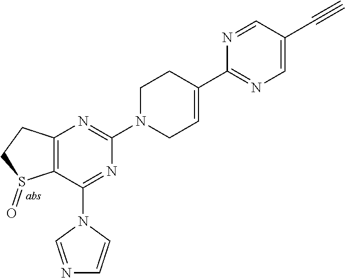 [SMILES: C#Cc1cnc(C2=CCN(c3nc4c(c(-n5ccnc5)n3)[S@@](=O)CC4)CC2)nc1]

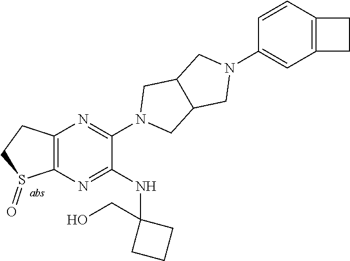 [SMILES: O=[S@]1CCc2nc(N3CC4CN(c5ccc6c(c5)CC6)CC4C3)c(NC3(CO)CCC3)nc21]

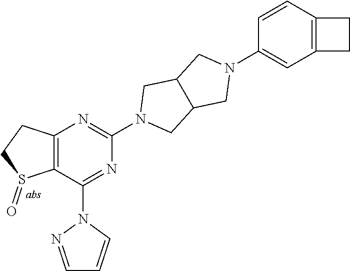 [SMILES: O=[S@]1CCc2nc(N3CC4CN(c5ccc6c(c5)CC6)CC4C3)nc(-n3cccn3)c21]

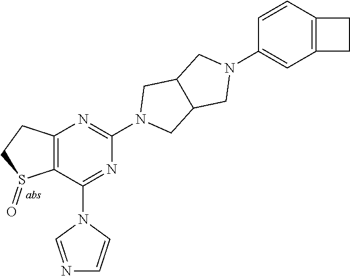 [SMILES: O=[S@]1CCc2nc(N3CC4CN(c5ccc6c(c5)CC6)CC4C3)nc(-n3ccnc3)c21]

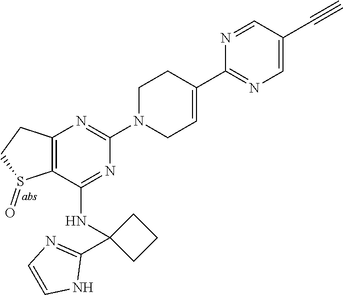 [SMILES: C#Cc1cnc(C2=CCN(c3nc4c(c(NC5(c6ncc[nH]6)CCC5)n3)[S@](=O)CC4)CC2)nc1]

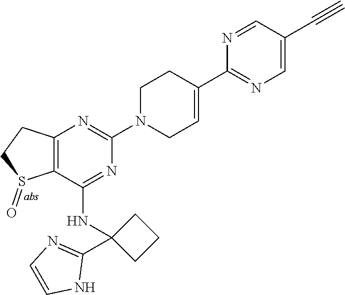 [SMILES: C#Cc1cnc(C2=CCN(c3nc4c(c(NC5(c6ncc[nH]6)CCC5)n3)[S@@](=O)CC4)CC2)nc1]

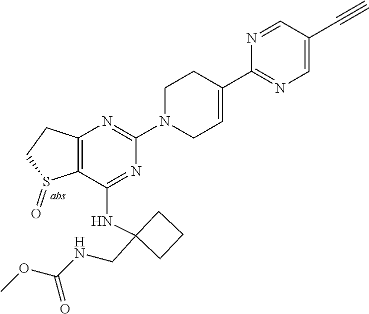 [SMILES: C#Cc1cnc(C2=CCN(c3nc4c(c(NC5(CNC(=O)OC)CCC5)n3)[S@](=O)CC4)CC2)nc1]

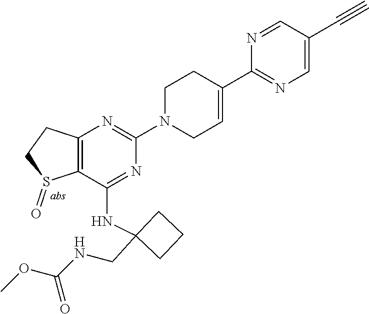 [SMILES: C#Cc1cnc(C2=CCN(c3nc4c(c(NC5(CNC(=O)OC)CCC5)n3)[S@@](=O)CC4)CC2)nc1]

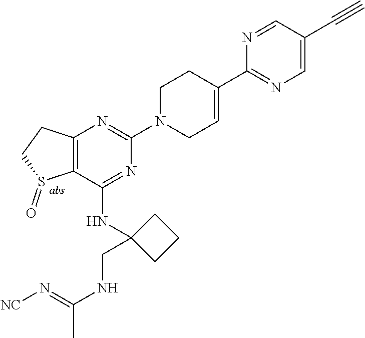 [SMILES: C#Cc1cnc(C2=CCN(c3nc4c(c(NC5(CN/C(C)=N/C#N)CCC5)n3)[S@](=O)CC4)CC2)nc1]

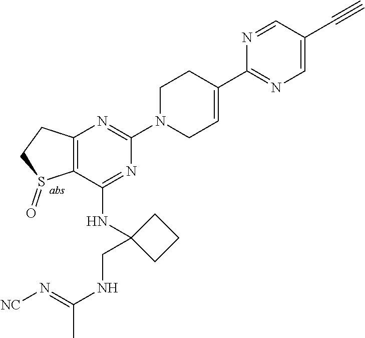 [SMILES: C#Cc1cnc(C2=CCN(c3nc4c(c(NC5(CN/C(C)=N/C#N)CCC5)n3)[S@@](=O)CC4)CC2)nc1]

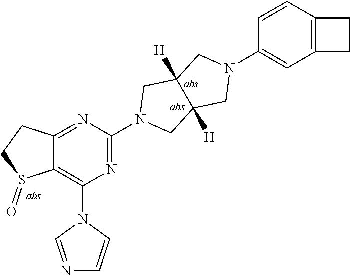 [SMILES: O=[S@]1CCc2nc(N3C[C@H]4CN(c5ccc6c(c5)CC6)C[C@H]4C3)nc(-n3ccnc3)c21]

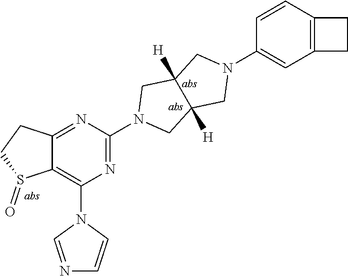 [SMILES: O=[S@@]1CCc2nc(N3C[C@H]4CN(c5ccc6c(c5)CC6)C[C@H]4C3)nc(-n3ccnc3)c21]

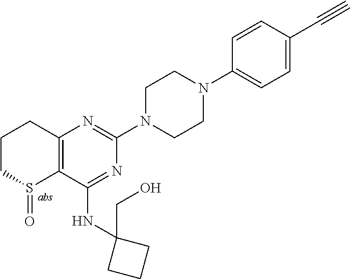 [SMILES: C#Cc1ccc(N2CCN(c3nc4c(c(NC5(CO)CCC5)n3)[S@](=O)CCC4)CC2)cc1]

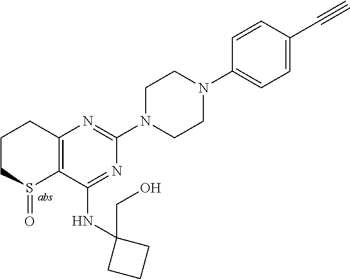 [SMILES: C#Cc1ccc(N2CCN(c3nc4c(c(NC5(CO)CCC5)n3)[S@@](=O)CCC4)CC2)cc1]

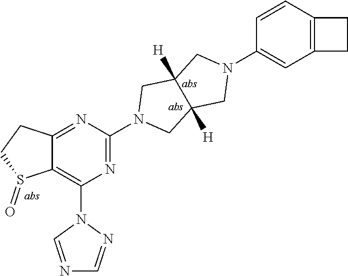 [SMILES: O=[S@@]1CCc2nc(N3C[C@H]4CN(c5ccc6c(c5)CC6)C[C@H]4C3)nc(-n3cncn3)c21]

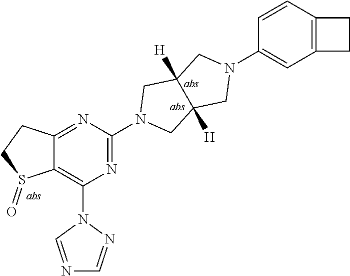 [SMILES: O=[S@]1CCc2nc(N3C[C@H]4CN(c5ccc6c(c5)CC6)C[C@H]4C3)nc(-n3cncn3)c21]

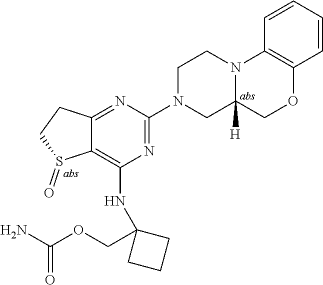 [SMILES: NC(=O)OCC1(Nc2nc(N3CCN4c5ccccc5OC[C@@H]4C3)nc3c2[S@](=O)CC3)CCC1]

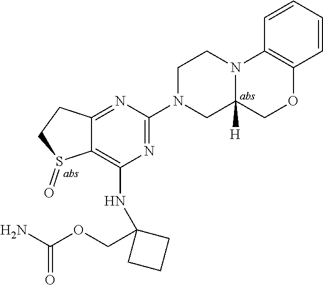 [SMILES: NC(=O)OCC1(Nc2nc(N3CCN4c5ccccc5OC[C@@H]4C3)nc3c2[S@@](=O)CC3)CCC1]

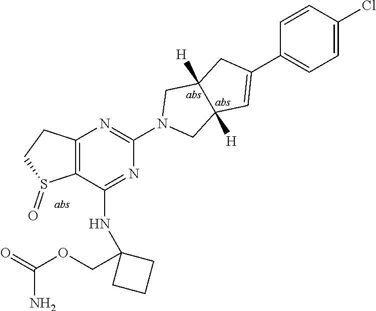 [SMILES: NC(=O)OCC1(Nc2nc(N3C[C@@H]4CC(c5ccc(Cl)cc5)=C[C@@H]4C3)nc3c2[S@](=O)CC3)CCC1]

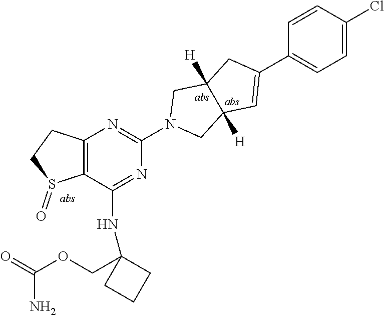 [SMILES: NC(=O)OCC1(Nc2nc(N3C[C@@H]4CC(c5ccc(Cl)cc5)=C[C@@H]4C3)nc3c2[S@@](=O)CC3)CCC1]

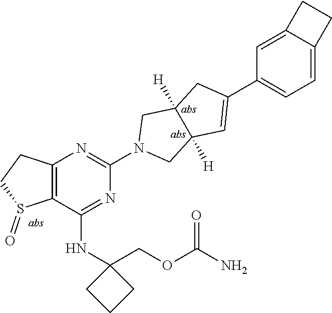 [SMILES: NC(=O)OCC1(Nc2nc(N3C[C@H]4CC(c5ccc6c(c5)CC6)=C[C@H]4C3)nc3c2[S@](=O)CC3)CCC1]

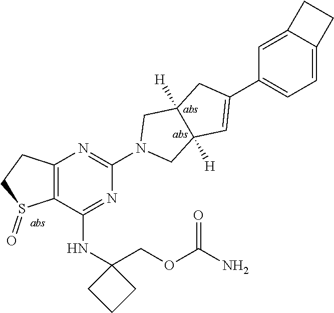 [SMILES: NC(=O)OCC1(Nc2nc(N3C[C@H]4CC(c5ccc6c(c5)CC6)=C[C@H]4C3)nc3c2[S@@](=O)CC3)CCC1]

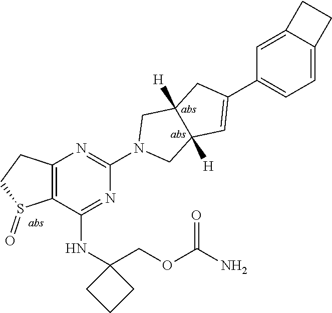 [SMILES: NC(=O)OCC1(Nc2nc(N3C[C@@H]4CC(c5ccc6c(c5)CC6)=C[C@@H]4C3)nc3c2[S@](=O)CC3)CCC1]

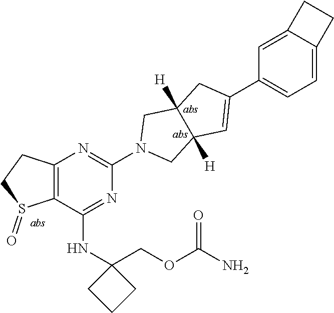 [SMILES: NC(=O)OCC1(Nc2nc(N3C[C@@H]4CC(c5ccc6c(c5)CC6)=C[C@@H]4C3)nc3c2[S@@](=O)CC3)CCC1]

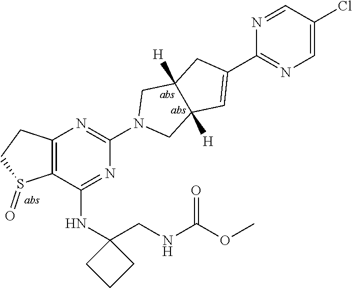 [SMILES: COC(=O)NCC1(Nc2nc(N3C[C@@H]4CC(c5ncc(Cl)cn5)=C[C@@H]4C3)nc3c2[S@](=O)CC3)CCC1]

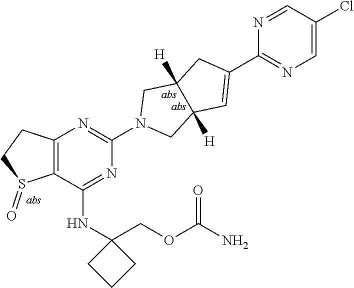 [SMILES: NC(=O)OCC1(Nc2nc(N3C[C@@H]4CC(c5ncc(Cl)cn5)=C[C@@H]4C3)nc3c2[S@@](=O)CC3)CCC1]

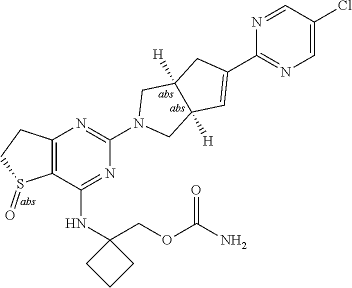 [SMILES: NC(=O)OCC1(Nc2nc(N3C[C@H]4CC(c5ncc(Cl)cn5)=C[C@H]4C3)nc3c2[S@](=O)CC3)CCC1]

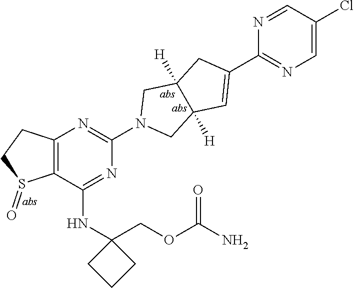 [SMILES: NC(=O)OCC1(Nc2nc(N3C[C@H]4CC(c5ncc(Cl)cn5)=C[C@H]4C3)nc3c2[S@@](=O)CC3)CCC1]

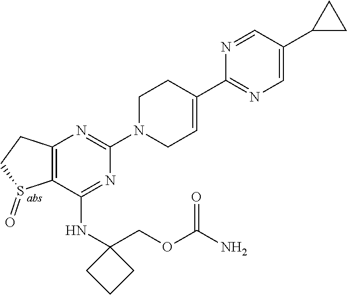 [SMILES: NC(=O)OCC1(Nc2nc(N3CC=C(c4ncc(C5CC5)cn4)CC3)nc3c2[S@](=O)CC3)CCC1]

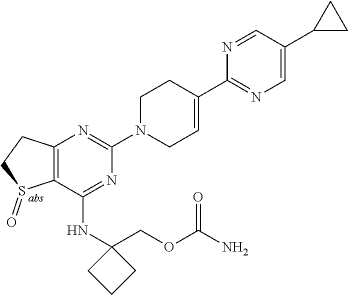 [SMILES: NC(=O)OCC1(Nc2nc(N3CC=C(c4ncc(C5CC5)cn4)CC3)nc3c2[S@@](=O)CC3)CCC1]

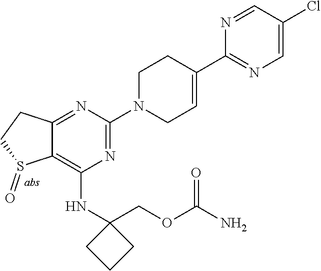 [SMILES: NC(=O)OCC1(Nc2nc(N3CC=C(c4ncc(Cl)cn4)CC3)nc3c2[S@](=O)CC3)CCC1]

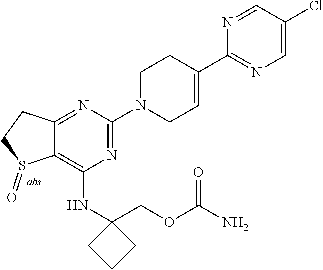 [SMILES: NC(=O)OCC1(Nc2nc(N3CC=C(c4ncc(Cl)cn4)CC3)nc3c2[S@@](=O)CC3)CCC1]

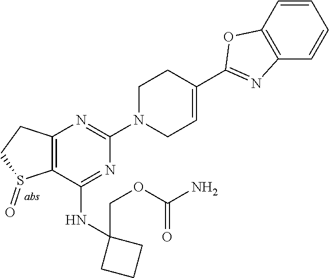 [SMILES: NC(=O)OCC1(Nc2nc(N3CC=C(c4nc5ccccc5o4)CC3)nc3c2[S@](=O)CC3)CCC1]

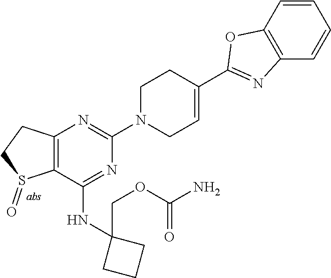 [SMILES: NC(=O)OCC1(Nc2nc(N3CC=C(c4nc5ccccc5o4)CC3)nc3c2[S@@](=O)CC3)CCC1]

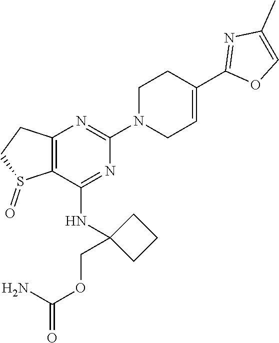 [SMILES: Cc1coc(C2=CCN(c3nc4c(c(NC5(COC(N)=O)CCC5)n3)[S@](=O)CC4)CC2)n1]

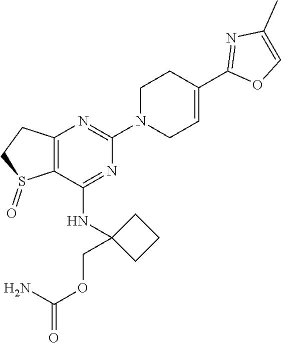 [SMILES: Cc1coc(C2=CCN(c3nc4c(c(NC5(COC(N)=O)CCC5)n3)[S@@](=O)CC4)CC2)n1]

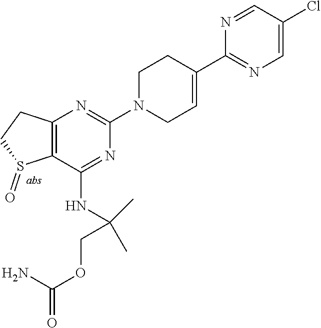 [SMILES: CC(C)(COC(N)=O)Nc1nc(N2CC=C(c3ncc(Cl)cn3)CC2)nc2c1[S@](=O)CC2]

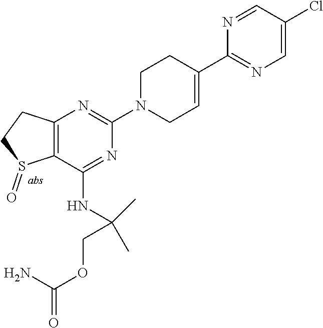 [SMILES: CC(C)(COC(N)=O)Nc1nc(N2CC=C(c3ncc(Cl)cn3)CC2)nc2c1[S@@](=O)CC2]

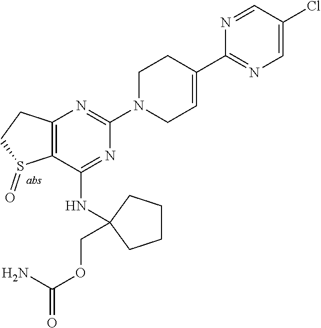 [SMILES: NC(=O)OCC1(Nc2nc(N3CC=C(c4ncc(Cl)cn4)CC3)nc3c2[S@](=O)CC3)CCCC1]

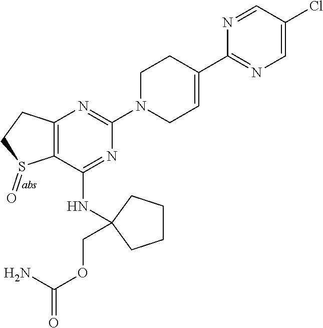 [SMILES: NC(=O)OCC1(Nc2nc(N3CC=C(c4ncc(Cl)cn4)CC3)nc3c2[S@@](=O)CC3)CCCC1]

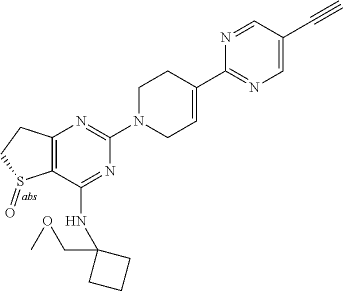 [SMILES: C#Cc1cnc(C2=CCN(c3nc4c(c(NC5(COC)CCC5)n3)[S@](=O)CC4)CC2)nc1]

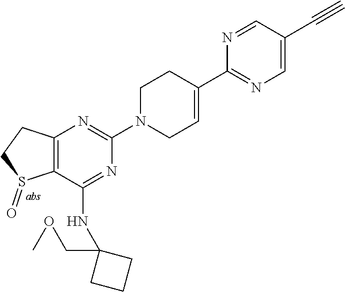 [SMILES: C#Cc1cnc(C2=CCN(c3nc4c(c(NC5(COC)CCC5)n3)[S@@](=O)CC4)CC2)nc1]

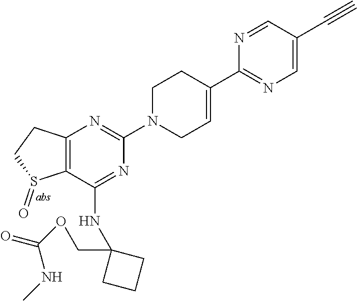 [SMILES: C#Cc1cnc(C2=CCN(c3nc4c(c(NC5(COC(=O)NC)CCC5)n3)[S@](=O)CC4)CC2)nc1]

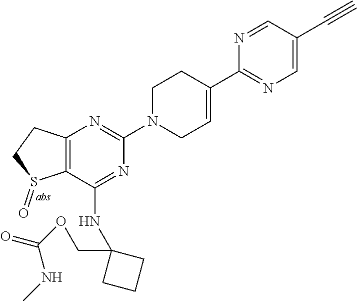 [SMILES: C#Cc1cnc(C2=CCN(c3nc4c(c(NC5(COC(=O)NC)CCC5)n3)[S@@](=O)CC4)CC2)nc1]

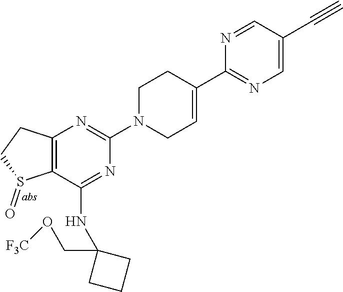 [SMILES: C#Cc1cnc(C2=CCN(c3nc4c(c(NC5(COC(F)(F)F)CCC5)n3)[S@](=O)CC4)CC2)nc1]

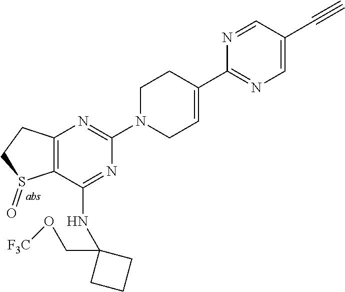 [SMILES: C#Cc1cnc(C2=CCN(c3nc4c(c(NC5(COC(F)(F)F)CCC5)n3)[S@@](=O)CC4)CC2)nc1]

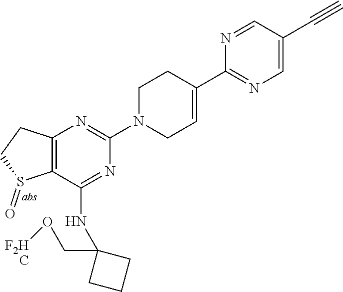 [SMILES: C#Cc1cnc(C2=CCN(c3nc4c(c(NC5(COC(F)F)CCC5)n3)[S@](=O)CC4)CC2)nc1]

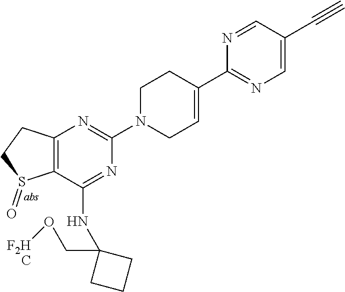 [SMILES: C#Cc1cnc(C2=CCN(c3nc4c(c(NC5(COC(F)F)CCC5)n3)[S@@](=O)CC4)CC2)nc1]

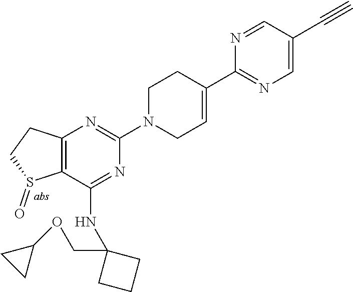 [SMILES: C#Cc1cnc(C2=CCN(c3nc4c(c(NC5(COC6CC6)CCC5)n3)[S@](=O)CC4)CC2)nc1]

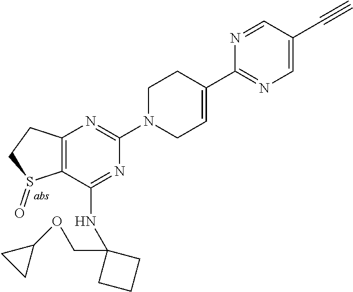 [SMILES: C#Cc1cnc(C2=CCN(c3nc4c(c(NC5(COC6CC6)CCC5)n3)[S@@](=O)CC4)CC2)nc1]

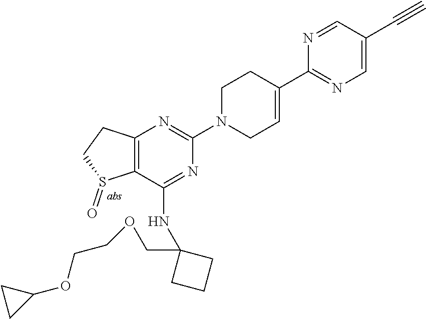 [SMILES: C#Cc1cnc(C2=CCN(c3nc4c(c(NC5(COCCOC6CC6)CCC5)n3)[S@](=O)CC4)CC2)nc1]

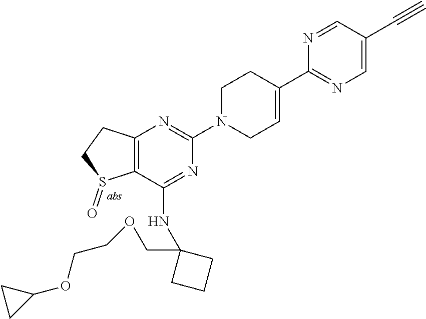 [SMILES: C#Cc1cnc(C2=CCN(c3nc4c(c(NC5(COCCOC6CC6)CCC5)n3)[S@@](=O)CC4)CC2)nc1]

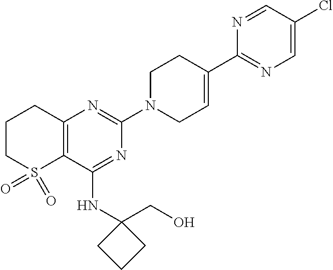 [SMILES: O=S1(=O)CCCc2nc(N3CC=C(c4ncc(Cl)cn4)CC3)nc(NC3(CO)CCC3)c21]

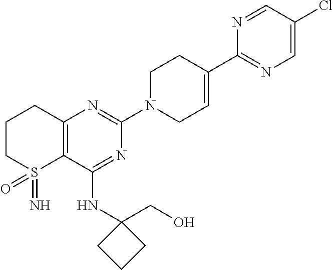 [SMILES: N=S1(=O)CCCc2nc(N3CC=C(c4ncc(Cl)cn4)CC3)nc(NC3(CO)CCC3)c21]

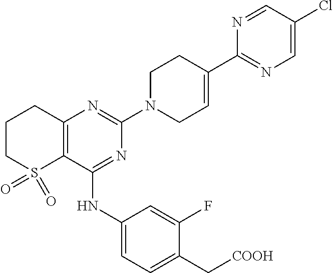 [SMILES: O=C(O)Cc1ccc(Nc2nc(N3CC=C(c4ncc(Cl)cn4)CC3)nc3c2S(=O)(=O)CCC3)cc1F]

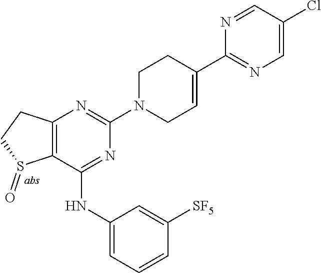

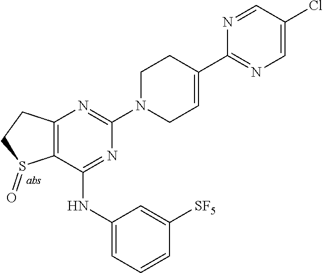

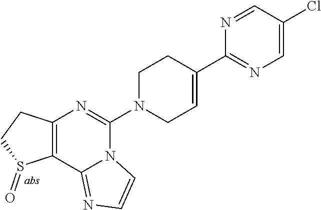 [SMILES: O=[S@@]1CCc2nc(N3CC=C(c4ncc(Cl)cn4)CC3)n3ccnc3c21]

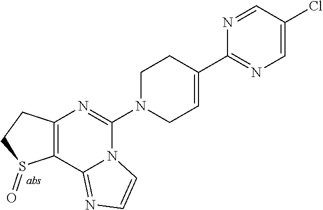 [SMILES: O=[S@]1CCc2nc(N3CC=C(c4ncc(Cl)cn4)CC3)n3ccnc3c21]

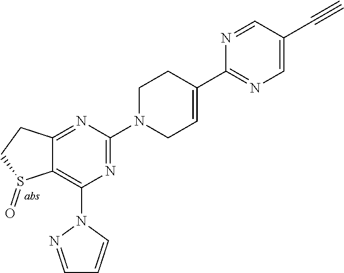 [SMILES: C#Cc1cnc(C2=CCN(c3nc4c(c(-n5cccn5)n3)[S@](=O)CC4)CC2)nc1]

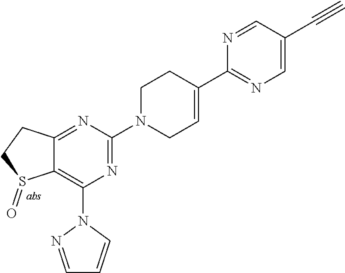 [SMILES: C#Cc1cnc(C2=CCN(c3nc4c(c(-n5cccn5)n3)[S@@](=O)CC4)CC2)nc1]

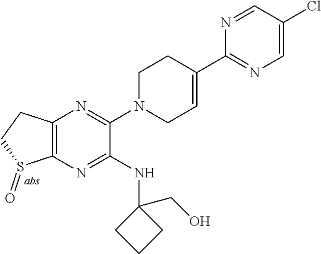 [SMILES: O=[S@@]1CCc2nc(N3CC=C(c4ncc(Cl)cn4)CC3)c(NC3(CO)CCC3)nc21]

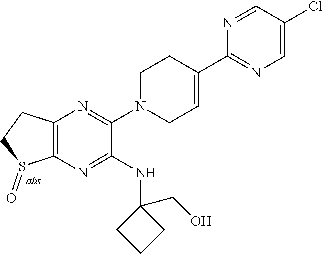 [SMILES: O=[S@]1CCc2nc(N3CC=C(c4ncc(Cl)cn4)CC3)c(NC3(CO)CCC3)nc21]

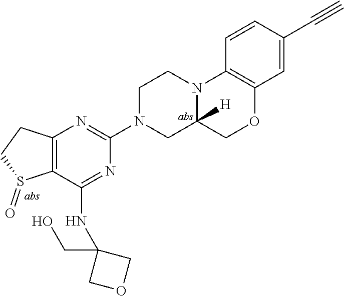

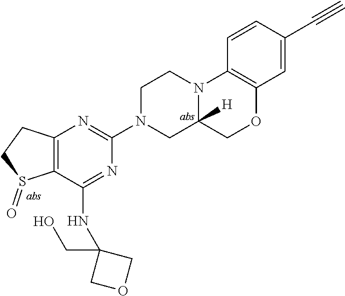

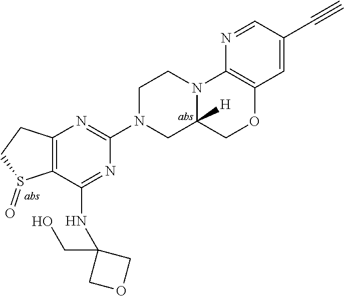

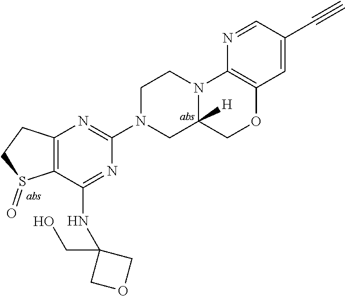

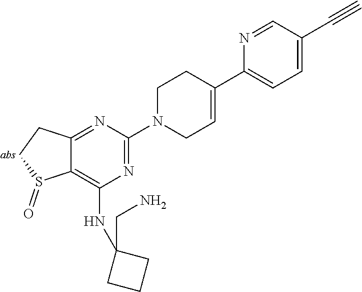

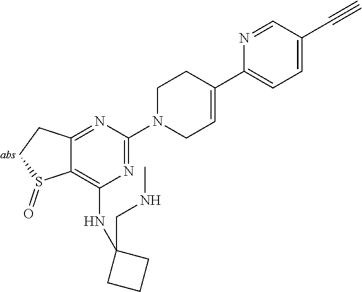

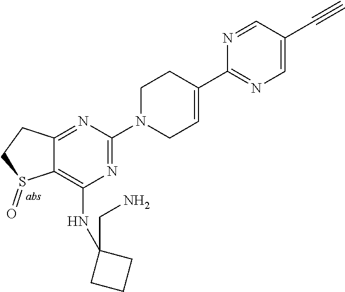 [SMILES: C#Cc1cnc(C2=CCN(c3nc4c(c(NC5(CN)CCC5)n3)[S@@](=O)CC4)CC2)nc1]

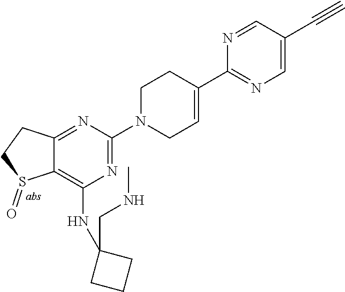 [SMILES: C#Cc1cnc(C2=CCN(c3nc4c(c(NC5(CNC)CCC5)n3)[S@@](=O)CC4)CC2)nc1]

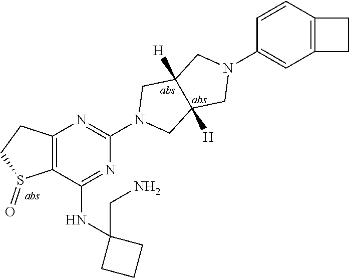 [SMILES: NCC1(Nc2nc(N3C[C@H]4CN(c5ccc6c(c5)CC6)C[C@H]4C3)nc3c2[S@](=O)CC3)CCC1]

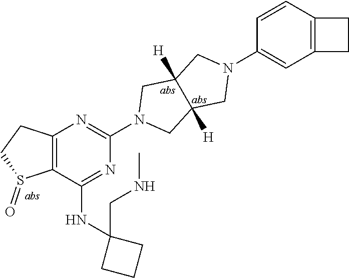 [SMILES: CNCC1(Nc2nc(N3C[C@H]4CN(c5ccc6c(c5)CC6)C[C@H]4C3)nc3c2[S@](=O)CC3)CCC1]

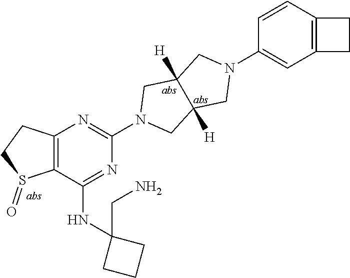 [SMILES: NCC1(Nc2nc(N3C[C@H]4CN(c5ccc6c(c5)CC6)C[C@H]4C3)nc3c2[S@@](=O)CC3)CCC1]

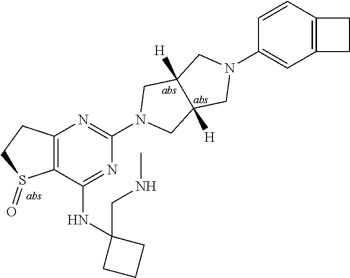 [SMILES: CNCC1(Nc2nc(N3C[C@H]4CN(c5ccc6c(c5)CC6)C[C@H]4C3)nc3c2[S@@](=O)CC3)CCC1]

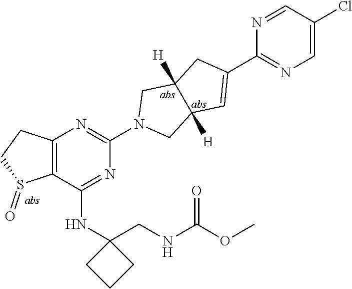 [SMILES: COC(=O)NCC1(Nc2nc(N3C[C@@H]4CC(c5ncc(Cl)cn5)=C[C@@H]4C3)nc3c2[S@](=O)CC3)CCC1]

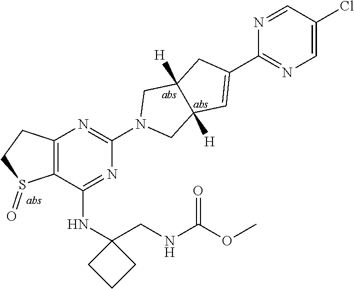 [SMILES: COC(=O)NCC1(Nc2nc(N3C[C@@H]4CC(c5ncc(Cl)cn5)=C[C@@H]4C3)nc3c2[S@@](=O)CC3)CCC1]

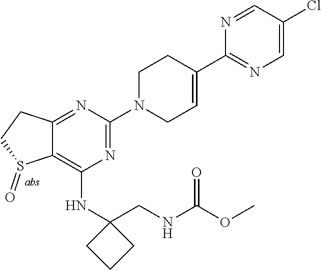 [SMILES: COC(=O)NCC1(Nc2nc(N3CC=C(c4ncc(Cl)cn4)CC3)nc3c2[S@](=O)CC3)CCC1]

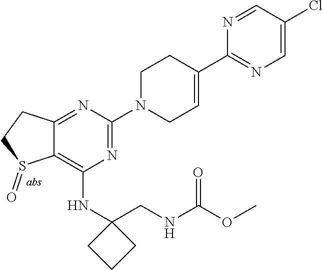 [SMILES: COC(=O)NCC1(Nc2nc(N3CC=C(c4ncc(Cl)cn4)CC3)nc3c2[S@@](=O)CC3)CCC1]

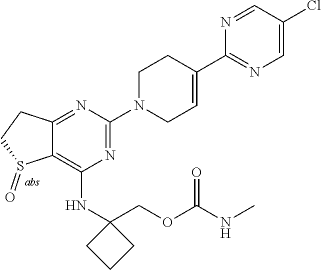 [SMILES: CNC(=O)OCC1(Nc2nc(N3CC=C(c4ncc(Cl)cn4)CC3)nc3c2[S@](=O)CC3)CCC1]

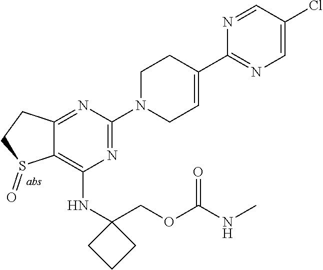 [SMILES: CNC(=O)OCC1(Nc2nc(N3CC=C(c4ncc(Cl)cn4)CC3)nc3c2[S@@](=O)CC3)CCC1]

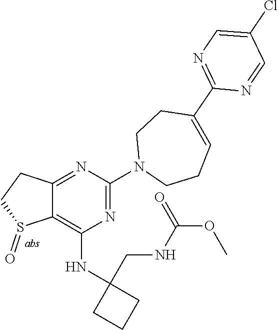 [SMILES: COC(=O)NCC1(Nc2nc(N3CCC=C(c4ncc(Cl)cn4)CC3)nc3c2[S@](=O)CC3)CCC1]

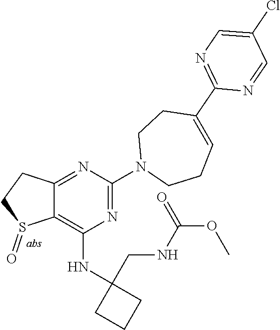 [SMILES: COC(=O)NCC1(Nc2nc(N3CCC=C(c4ncc(Cl)cn4)CC3)nc3c2[S@@](=O)CC3)CCC1]

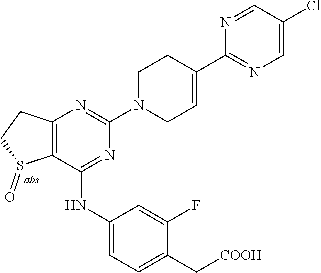 [SMILES: O=C(O)Cc1ccc(Nc2nc(N3CC=C(c4ncc(Cl)cn4)CC3)nc3c2[S@](=O)CC3)cc1F]

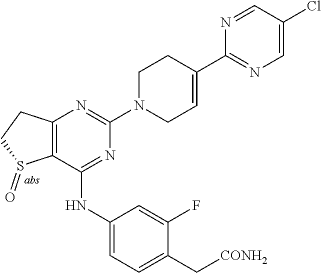 [SMILES: NC(=O)Cc1ccc(Nc2nc(N3CC=C(c4ncc(Cl)cn4)CC3)nc3c2[S@](=O)CC3)cc1F]

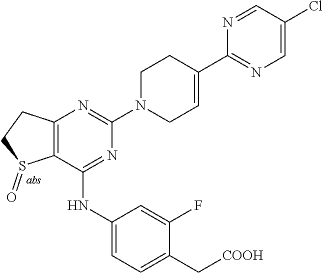 [SMILES: O=C(O)Cc1ccc(Nc2nc(N3CC=C(c4ncc(Cl)cn4)CC3)nc3c2[S@@](=O)CC3)cc1F]

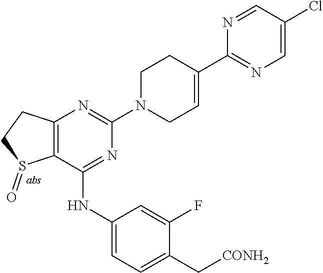 [SMILES: NC(=O)Cc1ccc(Nc2nc(N3CC=C(c4ncc(Cl)cn4)CC3)nc3c2[S@@](=O)CC3)cc1F]

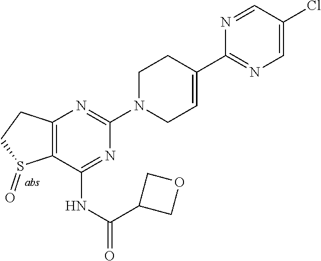 [SMILES: O=C(Nc1nc(N2CC=C(c3ncc(Cl)cn3)CC2)nc2c1[S@](=O)CC2)C1COC1]

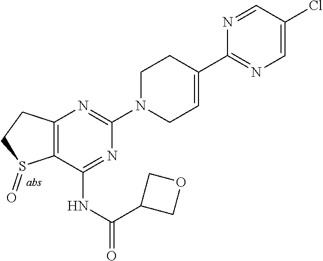 [SMILES: O=C(Nc1nc(N2CC=C(c3ncc(Cl)cn3)CC2)nc2c1[S@@](=O)CC2)C1COC1]

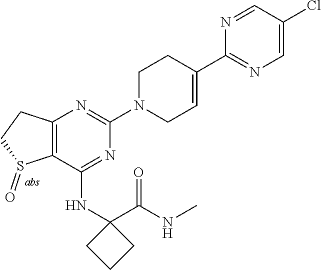 [SMILES: CNC(=O)C1(Nc2nc(N3CC=C(c4ncc(Cl)cn4)CC3)nc3c2[S@](=O)CC3)CCC1]

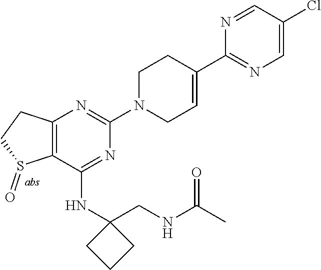 [SMILES: CC(=O)NCC1(Nc2nc(N3CC=C(c4ncc(Cl)cn4)CC3)nc3c2[S@](=O)CC3)CCC1]

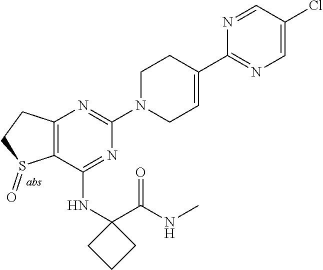 [SMILES: CNC(=O)C1(Nc2nc(N3CC=C(c4ncc(Cl)cn4)CC3)nc3c2[S@@](=O)CC3)CCC1]

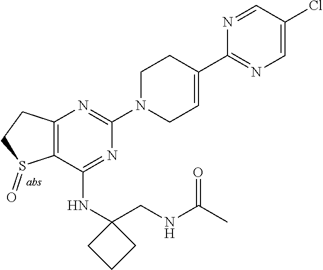 [SMILES: CC(=O)NCC1(Nc2nc(N3CC=C(c4ncc(Cl)cn4)CC3)nc3c2[S@@](=O)CC3)CCC1]

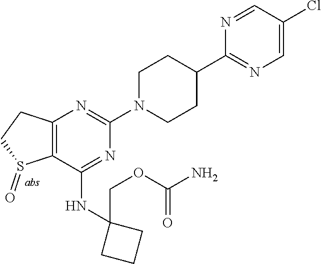 [SMILES: NC(=O)OCC1(Nc2nc(N3CCC(c4ncc(Cl)cn4)CC3)nc3c2[S@](=O)CC3)CCC1]

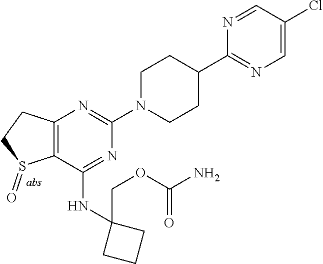 [SMILES: NC(=O)OCC1(Nc2nc(N3CCC(c4ncc(Cl)cn4)CC3)nc3c2[S@@](=O)CC3)CCC1]

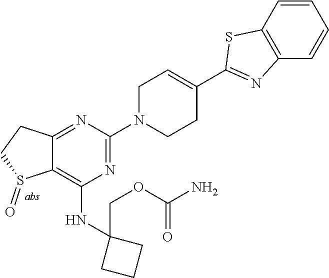 [SMILES: NC(=O)OCC1(Nc2nc(N3CC=C(c4nc5ccccc5s4)CC3)nc3c2[S@](=O)CC3)CCC1]

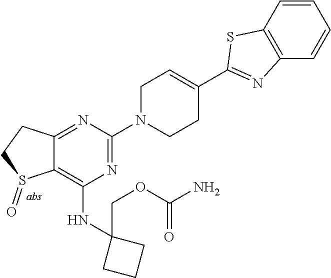 [SMILES: NC(=O)OCC1(Nc2nc(N3CC=C(c4nc5ccccc5s4)CC3)nc3c2[S@@](=O)CC3)CCC1]

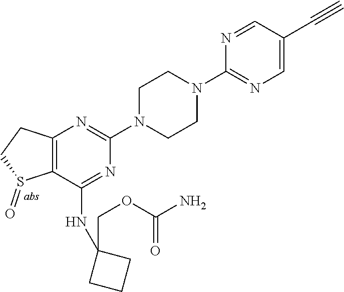 [SMILES: C#Cc1cnc(N2CCN(c3nc4c(c(NC5(COC(N)=O)CCC5)n3)[S@](=O)CC4)CC2)nc1]

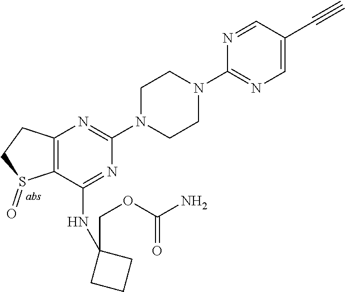 [SMILES: C#Cc1cnc(N2CCN(c3nc4c(c(NC5(COC(N)=O)CCC5)n3)[S@@](=O)CC4)CC2)nc1]

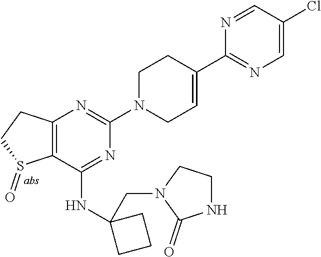 [SMILES: O=C1NCCN1CC1(Nc2nc(N3CC=C(c4ncc(Cl)cn4)CC3)nc3c2[S@](=O)CC3)CCC1]

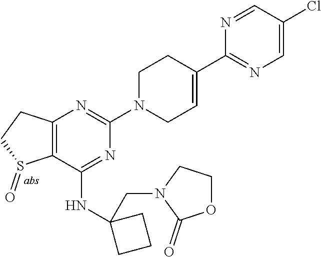 [SMILES: O=C1OCCN1CC1(Nc2nc(N3CC=C(c4ncc(Cl)cn4)CC3)nc3c2[S@](=O)CC3)CCC1]

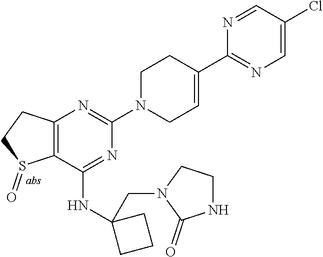 [SMILES: O=C1NCCN1CC1(Nc2nc(N3CC=C(c4ncc(Cl)cn4)CC3)nc3c2[S@@](=O)CC3)CCC1]

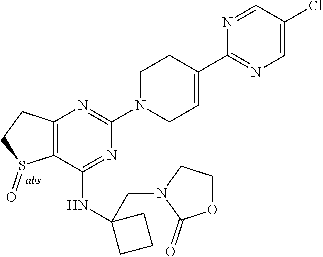 [SMILES: O=C1OCCN1CC1(Nc2nc(N3CC=C(c4ncc(Cl)cn4)CC3)nc3c2[S@@](=O)CC3)CCC1]

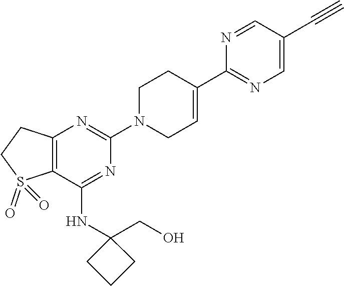 [SMILES: C#Cc1cnc(C2=CCN(c3nc4c(c(NC5(CO)CCC5)n3)S(=O)(=O)CC4)CC2)nc1]

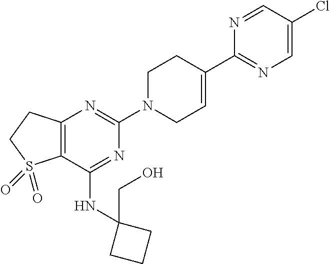 [SMILES: O=S1(=O)CCc2nc(N3CC=C(c4ncc(Cl)cn4)CC3)nc(NC3(CO)CCC3)c21]

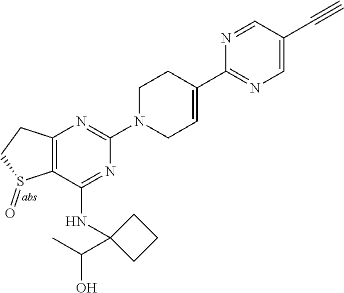 [SMILES: C#Cc1cnc(C2=CCN(c3nc4c(c(NC5(C(C)O)CCC5)n3)[S@](=O)CC4)CC2)nc1]

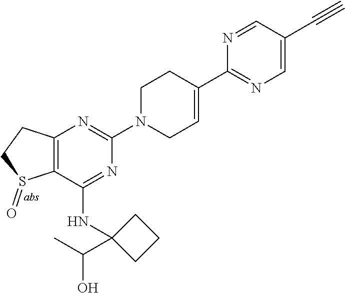 [SMILES: C#Cc1cnc(C2=CCN(c3nc4c(c(NC5(C(C)O)CCC5)n3)[S@@](=O)CC4)CC2)nc1]

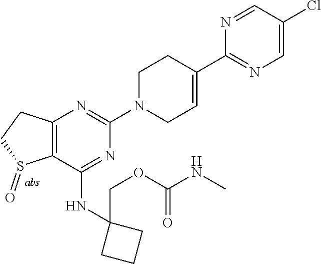 [SMILES: CNC(=O)OCC1(Nc2nc(N3CC=C(c4ncc(Cl)cn4)CC3)nc3c2[S@](=O)CC3)CCC1]

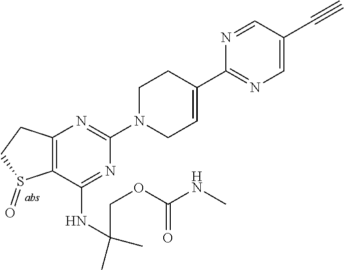 [SMILES: C#Cc1cnc(C2=CCN(c3nc4c(c(NC(C)(C)COC(=O)NC)n3)[S@](=O)CC4)CC2)nc1]

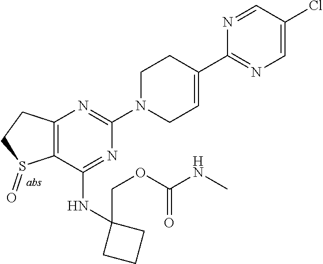 [SMILES: CNC(=O)OCC1(Nc2nc(N3CC=C(c4ncc(Cl)cn4)CC3)nc3c2[S@@](=O)CC3)CCC1]

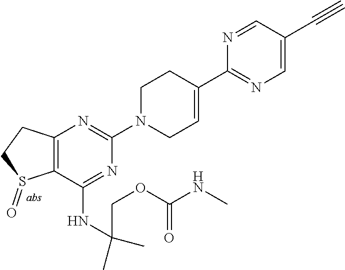 [SMILES: C#Cc1cnc(C2=CCN(c3nc4c(c(NC(C)(C)COC(=O)NC)n3)[S@@](=O)CC4)CC2)nc1]

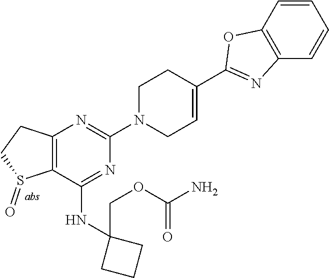 [SMILES: NC(=O)OCC1(Nc2nc(N3CC=C(c4nc5ccccc5o4)CC3)nc3c2[S@](=O)CC3)CCC1]

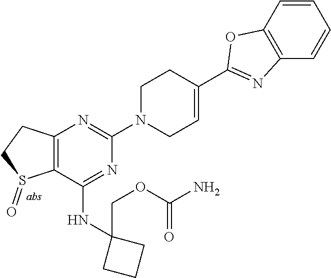 [SMILES: NC(=O)OCC1(Nc2nc(N3CC=C(c4nc5ccccc5o4)CC3)nc3c2[S@@](=O)CC3)CCC1]

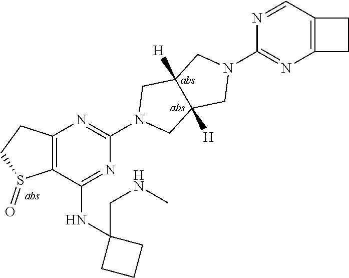 [SMILES: CNCC1(Nc2nc(N3C[C@H]4CN(c5ncc6c(n5)CC6)C[C@H]4C3)nc3c2[S@](=O)CC3)CCC1]

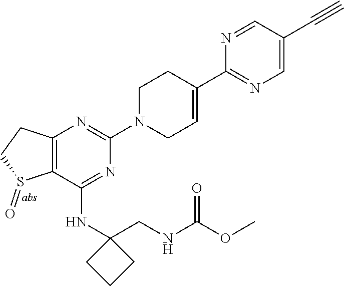 [SMILES: C#Cc1cnc(C2=CCN(c3nc4c(c(NC5(CNC(=O)OC)CCC5)n3)[S@](=O)CC4)CC2)nc1]

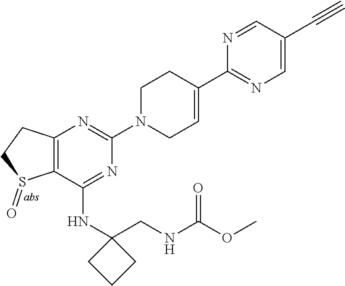 [SMILES: C#Cc1cnc(C2=CCN(c3nc4c(c(NC5(CNC(=O)OC)CCC5)n3)[S@@](=O)CC4)CC2)nc1]

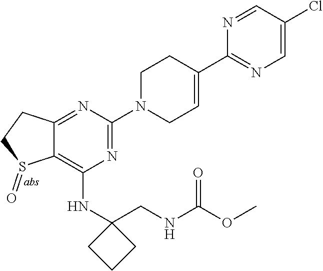 [SMILES: COC(=O)NCC1(Nc2nc(N3CC=C(c4ncc(Cl)cn4)CC3)nc3c2[S@@](=O)CC3)CCC1]

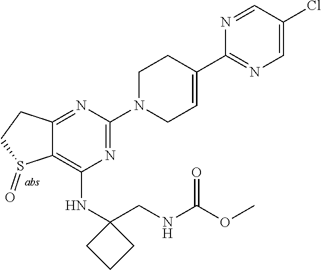 [SMILES: COC(=O)NCC1(Nc2nc(N3CC=C(c4ncc(Cl)cn4)CC3)nc3c2[S@](=O)CC3)CCC1]

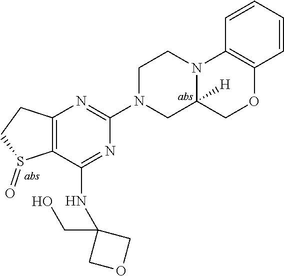

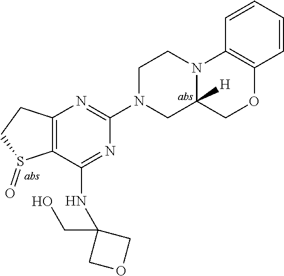

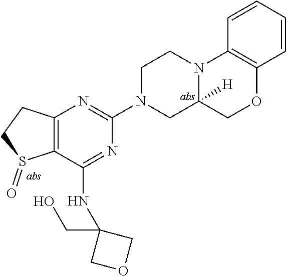

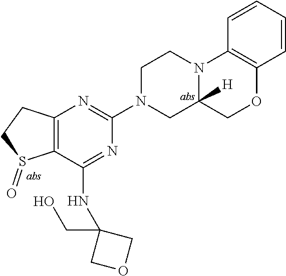

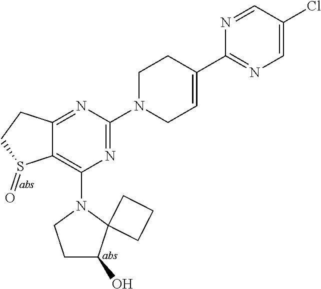 [SMILES: O=[S@@]1CCc2nc(N3CC=C(c4ncc(Cl)cn4)CC3)nc(N3CC[C@H](O)C34CCC4)c21]

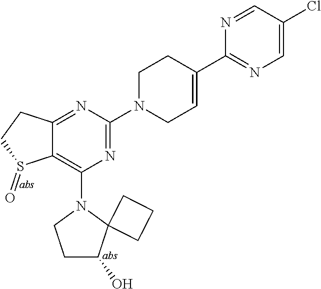 [SMILES: O=[S@@]1CCc2nc(N3CC=C(c4ncc(Cl)cn4)CC3)nc(N3CC[C@@H](O)C34CCC4)c21]

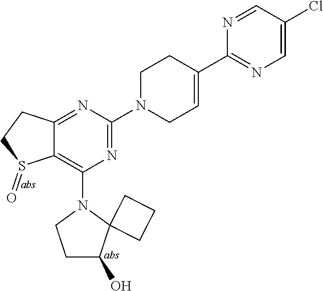 [SMILES: O=[S@]1CCc2nc(N3CC=C(c4ncc(Cl)cn4)CC3)nc(N3CC[C@H](O)C34CCC4)c21]

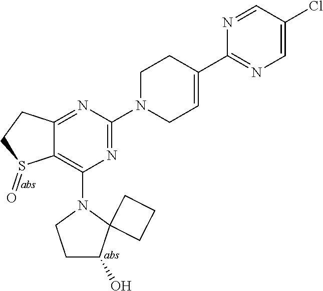 [SMILES: O=[S@]1CCc2nc(N3CC=C(c4ncc(Cl)cn4)CC3)nc(N3CC[C@@H](O)C34CCC4)c21]

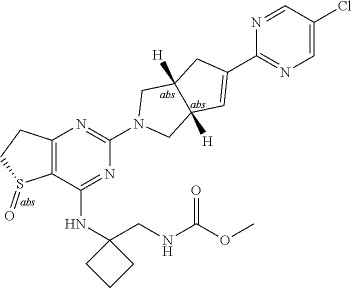 [SMILES: COC(=O)NCC1(Nc2nc(N3C[C@@H]4CC(c5ncc(Cl)cn5)=C[C@@H]4C3)nc3c2[S@](=O)CC3)CCC1]

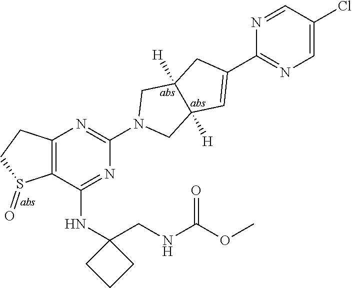 [SMILES: COC(=O)NCC1(Nc2nc(N3C[C@H]4CC(c5ncc(Cl)cn5)=C[C@H]4C3)nc3c2[S@](=O)CC3)CCC1]

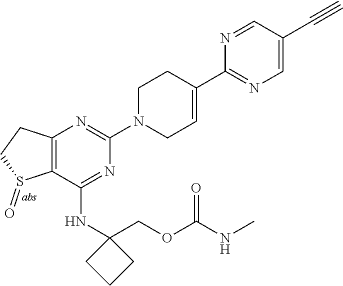 [SMILES: C#Cc1cnc(C2=CCN(c3nc4c(c(NC5(COC(=O)NC)CCC5)n3)[S@](=O)CC4)CC2)nc1]

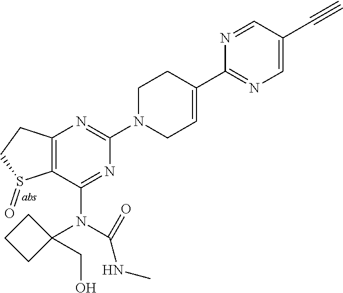 [SMILES: C#Cc1cnc(C2=CCN(c3nc4c(c(N(C(=O)NC)C5(CO)CCC5)n3)[S@](=O)CC4)CC2)nc1]

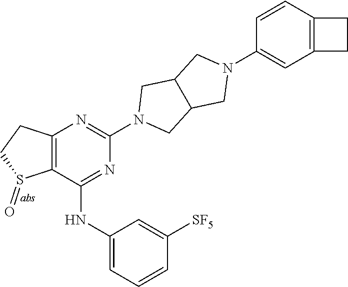

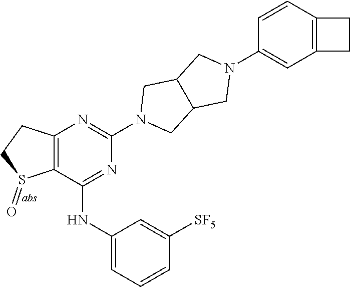

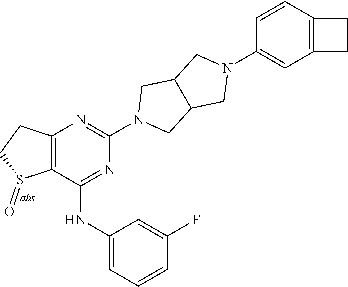 [SMILES: O=[S@@]1CCc2nc(N3CC4CN(c5ccc6c(c5)CC6)CC4C3)nc(Nc3cccc(F)c3)c21]

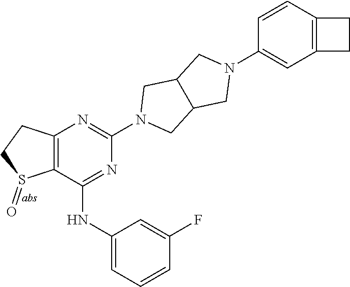 [SMILES: O=[S@]1CCc2nc(N3CC4CN(c5ccc6c(c5)CC6)CC4C3)nc(Nc3cccc(F)c3)c21]

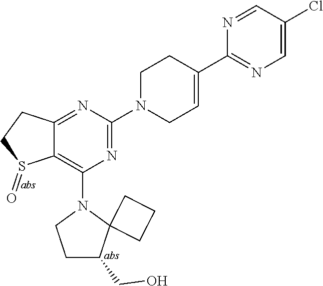 [SMILES: O=[S@]1CCc2nc(N3CC=C(c4ncc(Cl)cn4)CC3)nc(N3CC[C@@H](CO)C34CCC4)c21]

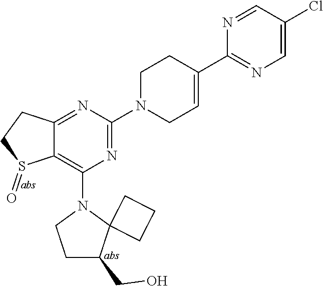 [SMILES: O=[S@]1CCc2nc(N3CC=C(c4ncc(Cl)cn4)CC3)nc(N3CC[C@H](CO)C34CCC4)c21]

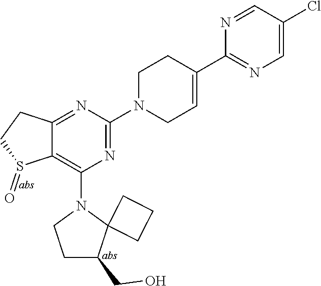 [SMILES: O=[S@@]1CCc2nc(N3CC=C(c4ncc(Cl)cn4)CC3)nc(N3CC[C@H](CO)C34CCC4)c21]

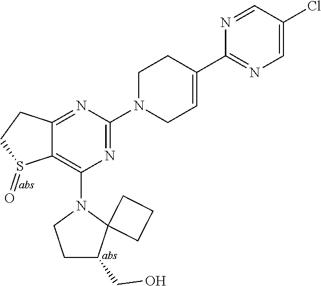 [SMILES: O=[S@@]1CCc2nc(N3CC=C(c4ncc(Cl)cn4)CC3)nc(N3CC[C@@H](CO)C34CCC4)c21]

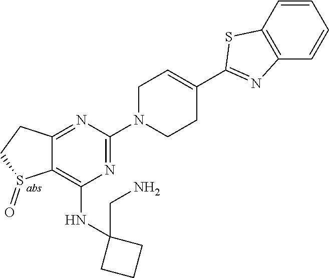 [SMILES: NCC1(Nc2nc(N3CC=C(c4nc5ccccc5s4)CC3)nc3c2[S@](=O)CC3)CCC1]

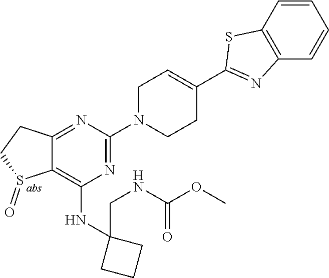 [SMILES: COC(=O)NCC1(Nc2nc(N3CC=C(c4nc5ccccc5s4)CC3)nc3c2[S@](=O)CC3)CCC1]

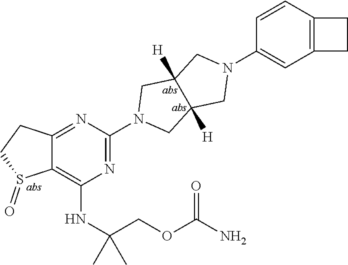 [SMILES: CC(C)(COC(N)=O)Nc1nc(N2C[C@H]3CN(c4ccc5c(c4)CC5)C[C@H]3C2)nc2c1[S@](=O)CC2]

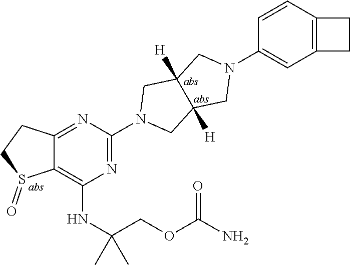 [SMILES: CC(C)(COC(N)=O)Nc1nc(N2C[C@H]3CN(c4ccc5c(c4)CC5)C[C@H]3C2)nc2c1[S@@](=O)CC2]

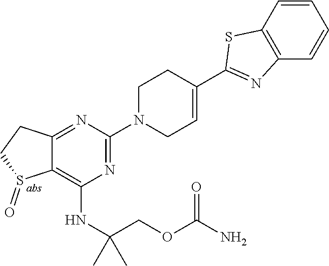 [SMILES: CC(C)(COC(N)=O)Nc1nc(N2CC=C(c3nc4ccccc4s3)CC2)nc2c1[S@](=O)CC2]

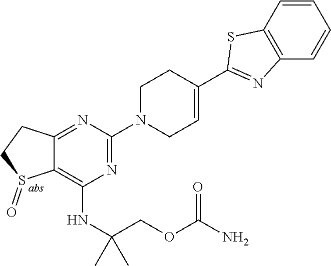 [SMILES: CC(C)(COC(N)=O)Nc1nc(N2CC=C(c3nc4ccccc4s3)CC2)nc2c1[S@@](=O)CC2]

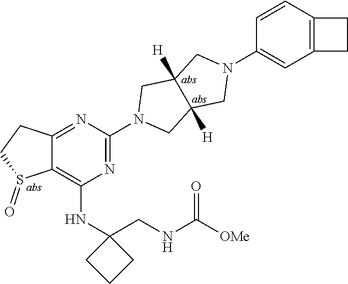 [SMILES: COC(=O)NCC1(Nc2nc(N3C[C@H]4CN(c5ccc6c(c5)CC6)C[C@H]4C3)nc3c2[S@](=O)CC3)CCC1]

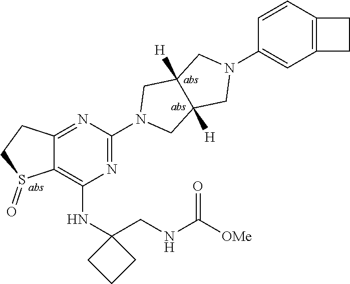 [SMILES: COC(=O)NCC1(Nc2nc(N3C[C@H]4CN(c5ccc6c(c5)CC6)C[C@H]4C3)nc3c2[S@@](=O)CC3)CCC1]

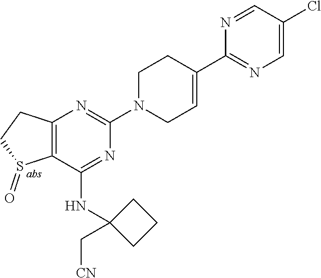 [SMILES: N#CCC1(Nc2nc(N3CC=C(c4ncc(Cl)cn4)CC3)nc3c2[S@](=O)CC3)CCC1]

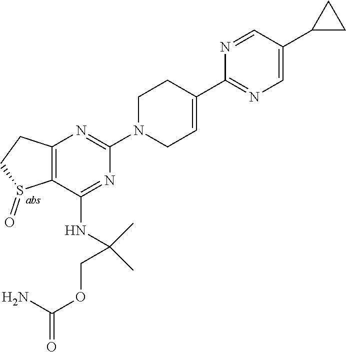 [SMILES: CC(C)(COC(N)=O)Nc1nc(N2CC=C(c3ncc(C4CC4)cn3)CC2)nc2c1[S@](=O)CC2]

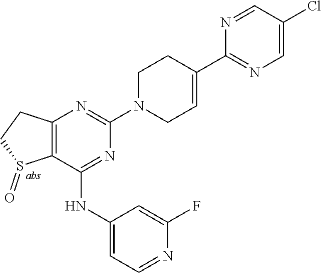 [SMILES: O=[S@@]1CCc2nc(N3CC=C(c4ncc(Cl)cn4)CC3)nc(Nc3ccnc(F)c3)c21]

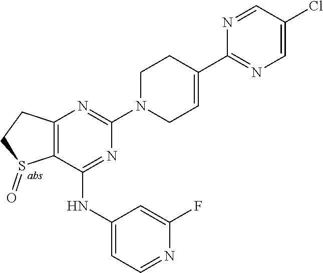 [SMILES: O=[S@]1CCc2nc(N3CC=C(c4ncc(Cl)cn4)CC3)nc(Nc3ccnc(F)c3)c21]

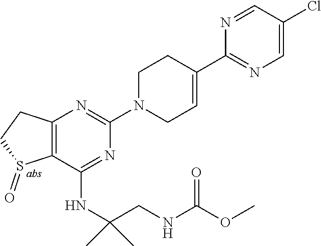 [SMILES: COC(=O)NCC(C)(C)Nc1nc(N2CC=C(c3ncc(Cl)cn3)CC2)nc2c1[S@](=O)CC2]

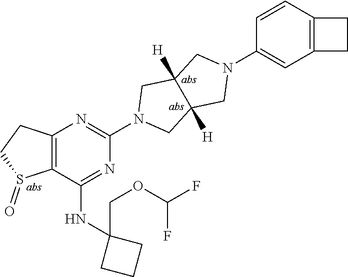 [SMILES: O=[S@@]1CCc2nc(N3C[C@H]4CN(c5ccc6c(c5)CC6)C[C@H]4C3)nc(NC3(COC(F)F)CCC3)c21]

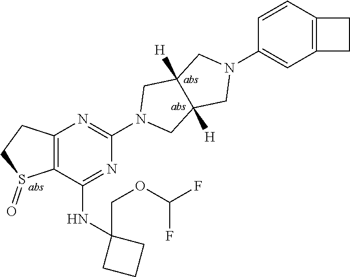 [SMILES: O=[S@]1CCc2nc(N3C[C@H]4CN(c5ccc6c(c5)CC6)C[C@H]4C3)nc(NC3(COC(F)F)CCC3)c21]

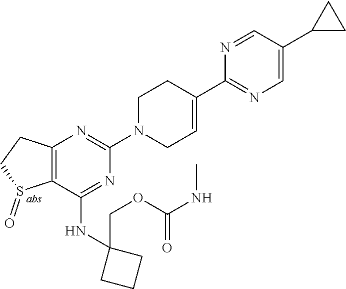 [SMILES: CNC(=O)OCC1(Nc2nc(N3CC=C(c4ncc(C5CC5)cn4)CC3)nc3c2[S@](=O)CC3)CCC1]

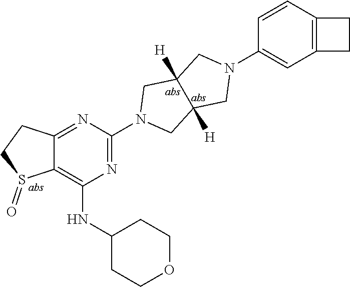 [SMILES: O=[S@]1CCc2nc(N3C[C@H]4CN(c5ccc6c(c5)CC6)C[C@H]4C3)nc(NC3CCOCC3)c21]

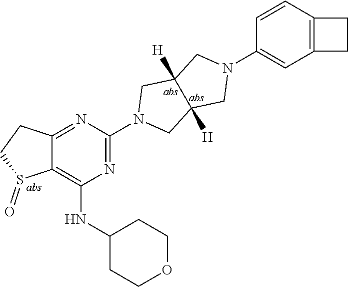 [SMILES: O=[S@@]1CCc2nc(N3C[C@H]4CN(c5ccc6c(c5)CC6)C[C@H]4C3)nc(NC3CCOCC3)c21]

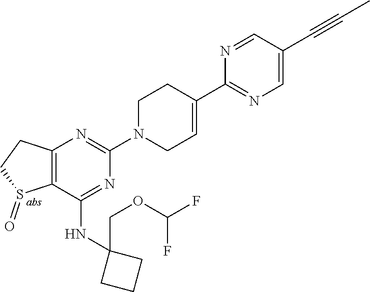 [SMILES: CC#Cc1cnc(C2=CCN(c3nc4c(c(NC5(COC(F)F)CCC5)n3)[S@](=O)CC4)CC2)nc1]

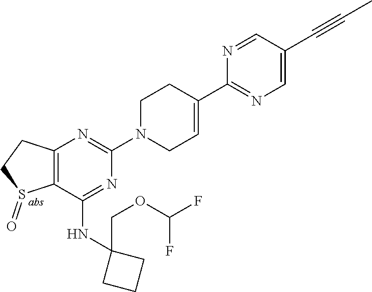 [SMILES: CC#Cc1cnc(C2=CCN(c3nc4c(c(NC5(COC(F)F)CCC5)n3)[S@@](=O)CC4)CC2)nc1]

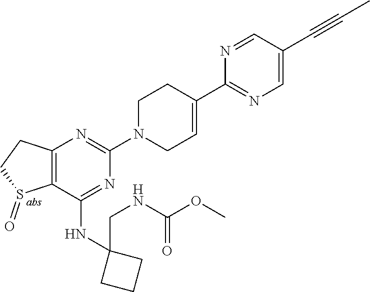 [SMILES: CC#Cc1cnc(C2=CCN(c3nc4c(c(NC5(CNC(=O)OC)CCC5)n3)[S@](=O)CC4)CC2)nc1]

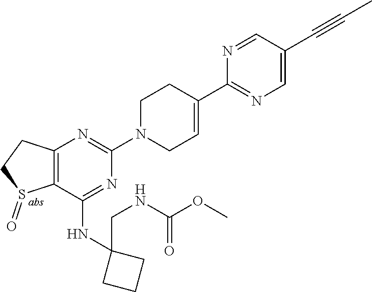 [SMILES: CC#Cc1cnc(C2=CCN(c3nc4c(c(NC5(CNC(=O)OC)CCC5)n3)[S@@](=O)CC4)CC2)nc1]

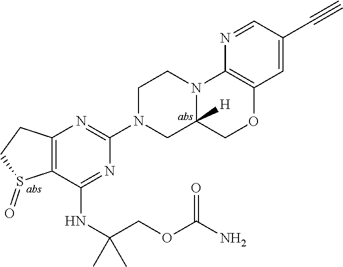

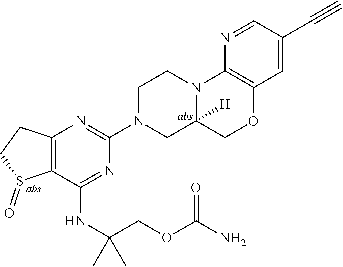

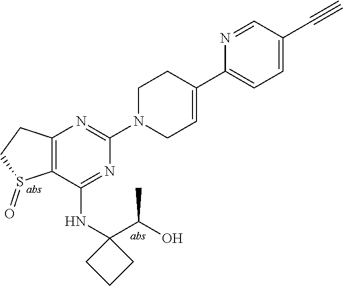 [SMILES: C#Cc1ccc(C2=CCN(c3nc4c(c(NC5([C@@H](C)O)CCC5)n3)[S@](=O)CC4)CC2)nc1]

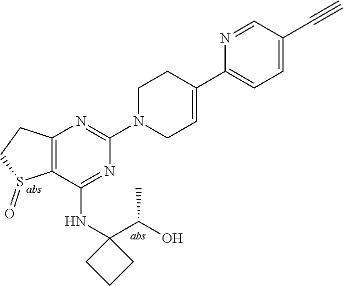 [SMILES: C#Cc1ccc(C2=CCN(c3nc4c(c(NC5([C@H](C)O)CCC5)n3)[S@](=O)CC4)CC2)nc1]

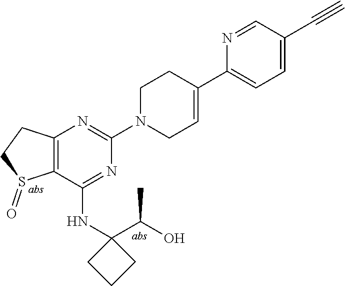 [SMILES: C#Cc1ccc(C2=CCN(c3nc4c(c(NC5([C@@H](C)O)CCC5)n3)[S@@](=O)CC4)CC2)nc1]

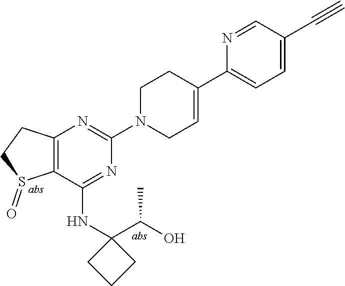 [SMILES: C#Cc1ccc(C2=CCN(c3nc4c(c(NC5([C@H](C)O)CCC5)n3)[S@@](=O)CC4)CC2)nc1]

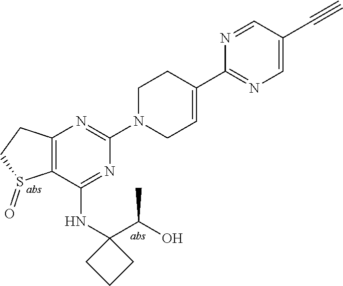 [SMILES: C#Cc1cnc(C2=CCN(c3nc4c(c(NC5([C@@H](C)O)CCC5)n3)[S@](=O)CC4)CC2)nc1]

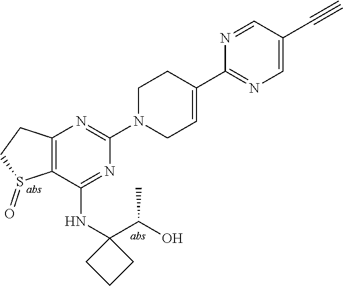 [SMILES: C#Cc1cnc(C2=CCN(c3nc4c(c(NC5([C@H](C)O)CCC5)n3)[S@](=O)CC4)CC2)nc1]

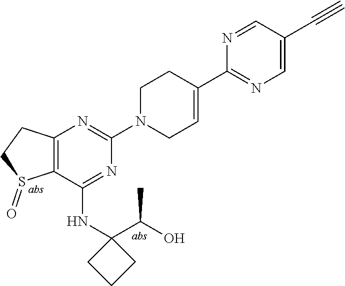 [SMILES: C#Cc1cnc(C2=CCN(c3nc4c(c(NC5([C@@H](C)O)CCC5)n3)[S@@](=O)CC4)CC2)nc1]

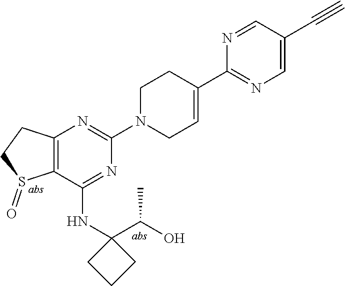 [SMILES: C#Cc1cnc(C2=CCN(c3nc4c(c(NC5([C@H](C)O)CCC5)n3)[S@@](=O)CC4)CC2)nc1]

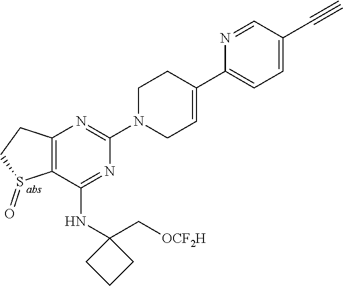

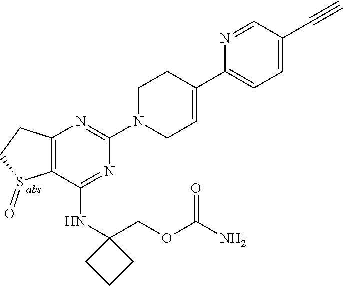 [SMILES: C#Cc1ccc(C2=CCN(c3nc4c(c(NC5(COC(N)=O)CCC5)n3)[S@](=O)CC4)CC2)nc1]

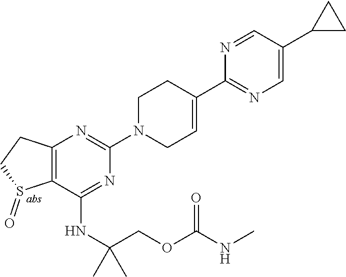 [SMILES: CNC(=O)OCC(C)(C)Nc1nc(N2CC=C(c3ncc(C4CC4)cn3)CC2)nc2c1[S@](=O)CC2]

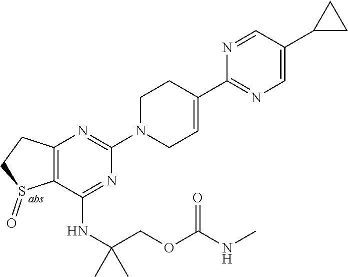 [SMILES: CNC(=O)OCC(C)(C)Nc1nc(N2CC=C(c3ncc(C4CC4)cn3)CC2)nc2c1[S@@](=O)CC2]

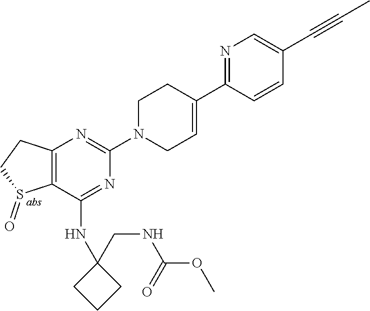 [SMILES: CC#Cc1ccc(C2=CCN(c3nc4c(c(NC5(CNC(=O)OC)CCC5)n3)[S@](=O)CC4)CC2)nc1]

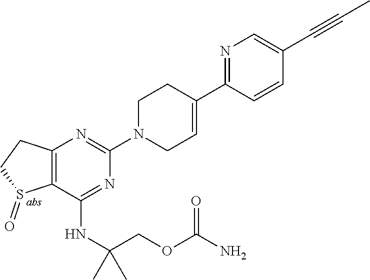 [SMILES: CC#Cc1ccc(C2=CCN(c3nc4c(c(NC(C)(C)COC(N)=O)n3)[S@](=O)CC4)CC2)nc1]

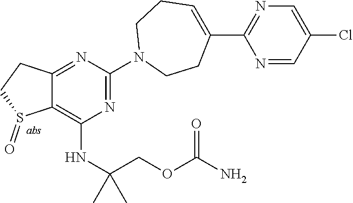 [SMILES: CC(C)(COC(N)=O)Nc1nc(N2CCC=C(c3ncc(Cl)cn3)CC2)nc2c1[S@](=O)CC2]

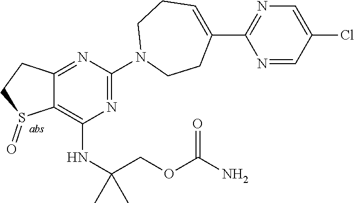 [SMILES: CC(C)(COC(N)=O)Nc1nc(N2CCC=C(c3ncc(Cl)cn3)CC2)nc2c1[S@@](=O)CC2]

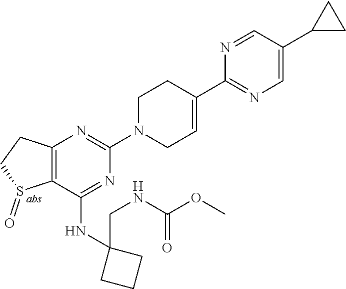 [SMILES: COC(=O)NCC1(Nc2nc(N3CC=C(c4ncc(C5CC5)cn4)CC3)nc3c2[S@](=O)CC3)CCC1]

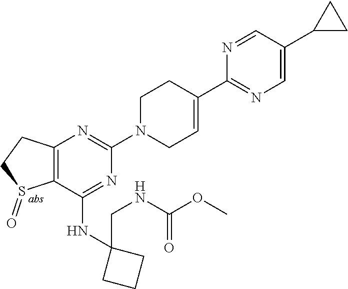 [SMILES: COC(=O)NCC1(Nc2nc(N3CC=C(c4ncc(C5CC5)cn4)CC3)nc3c2[S@@](=O)CC3)CCC1]

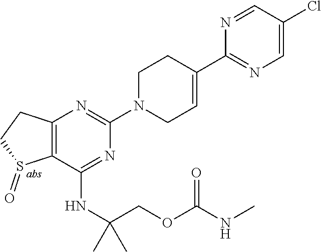 [SMILES: CNC(=O)OCC(C)(C)Nc1nc(N2CC=C(c3ncc(Cl)cn3)CC2)nc2c1[S@](=O)CC2]

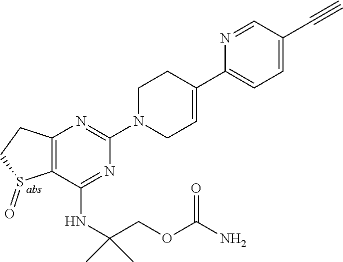 [SMILES: C#Cc1ccc(C2=CCN(c3nc4c(c(NC(C)(C)COC(N)=O)n3)[S@](=O)CC4)CC2)nc1]

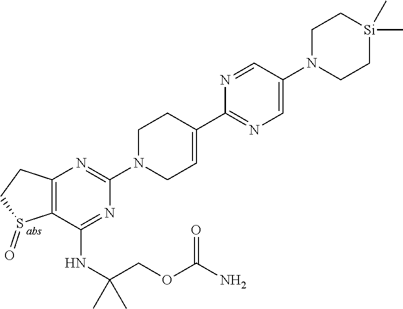 [SMILES: CC(C)(COC(N)=O)Nc1nc(N2CC=C(c3ncc(N4CC[Si](C)(C)CC4)cn3)CC2)nc2c1[S@](=O)CC2]

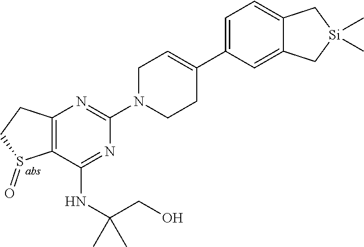 [SMILES: CC(C)(CO)Nc1nc(N2CC=C(c3ccc4c(c3)C[Si](C)(C)C4)CC2)nc2c1[S@](=O)CC2]

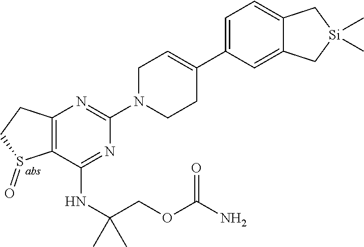 [SMILES: CC(C)(COC(N)=O)Nc1nc(N2CC=C(c3ccc4c(c3)C[Si](C)(C)C4)CC2)nc2c1[S@](=O)CC2]

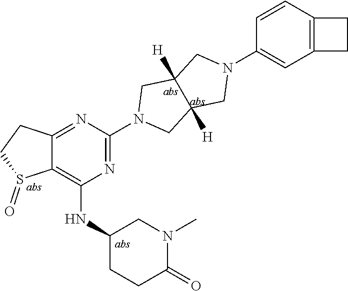 [SMILES: CN1C[C@H](Nc2nc(N3C[C@H]4CN(c5ccc6c(c5)CC6)C[C@H]4C3)nc3c2[S@](=O)CC3)CCC1=O]

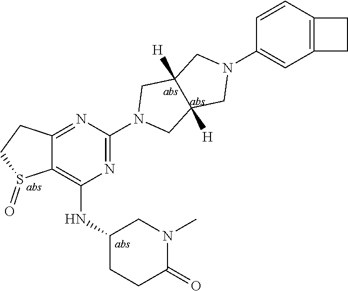 [SMILES: CN1C[C@@H](Nc2nc(N3C[C@H]4CN(c5ccc6c(c5)CC6)C[C@H]4C3)nc3c2[S@](=O)CC3)CCC1=O]

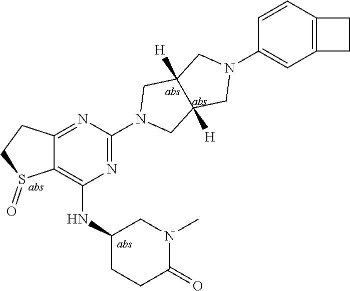 [SMILES: CN1C[C@H](Nc2nc(N3C[C@H]4CN(c5ccc6c(c5)CC6)C[C@H]4C3)nc3c2[S@@](=O)CC3)CCC1=O]

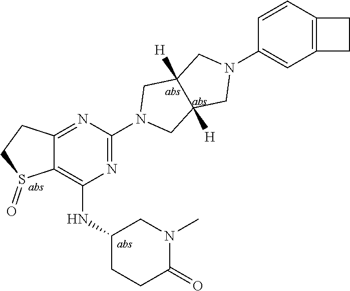 [SMILES: CN1C[C@@H](Nc2nc(N3C[C@H]4CN(c5ccc6c(c5)CC6)C[C@H]4C3)nc3c2[S@@](=O)CC3)CCC1=O]

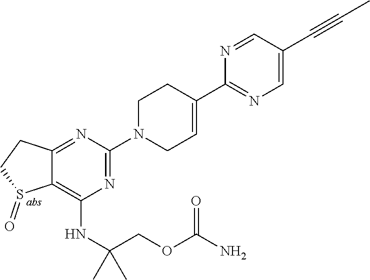 [SMILES: CC#Cc1cnc(C2=CCN(c3nc4c(c(NC(C)(C)COC(N)=O)n3)[S@](=O)CC4)CC2)nc1]

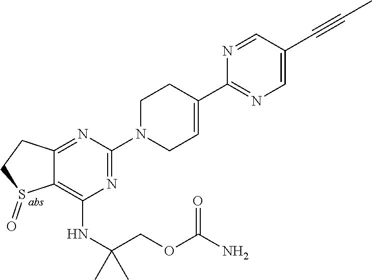 [SMILES: CC#Cc1cnc(C2=CCN(c3nc4c(c(NC(C)(C)COC(N)=O)n3)[S@@](=O)CC4)CC2)nc1]

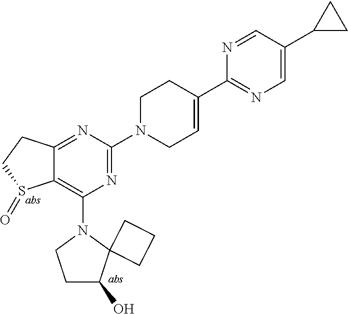 [SMILES: O=[S@@]1CCc2nc(N3CC=C(c4ncc(C5CC5)cn4)CC3)nc(N3CC[C@H](O)C34CCC4)c21]

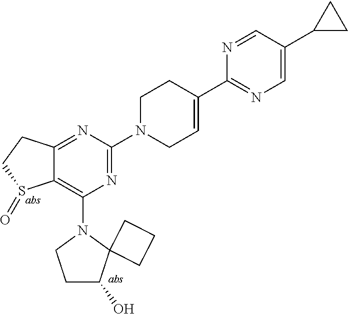 [SMILES: O=[S@@]1CCc2nc(N3CC=C(c4ncc(C5CC5)cn4)CC3)nc(N3CC[C@@H](O)C34CCC4)c21]

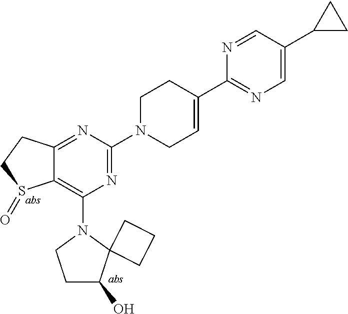 [SMILES: O=[S@]1CCc2nc(N3CC=C(c4ncc(C5CC5)cn4)CC3)nc(N3CC[C@H](O)C34CCC4)c21]

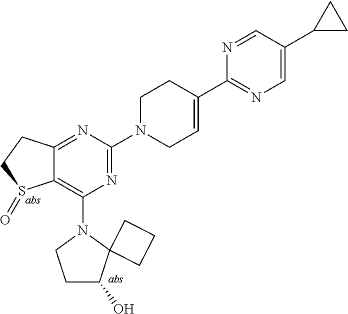 [SMILES: O=[S@]1CCc2nc(N3CC=C(c4ncc(C5CC5)cn4)CC3)nc(N3CC[C@@H](O)C34CCC4)c21]

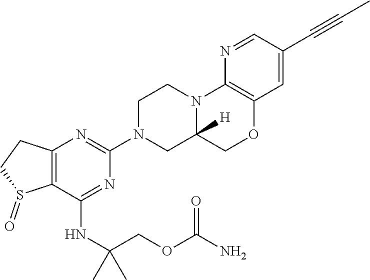

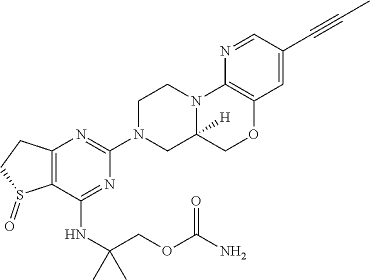

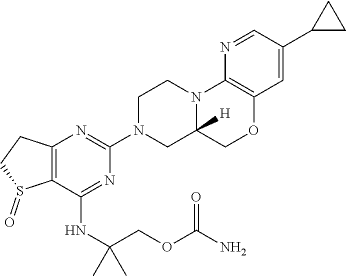

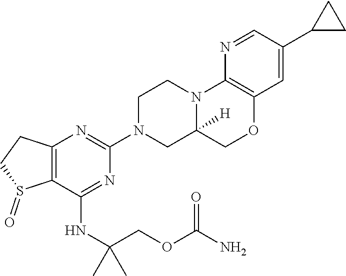

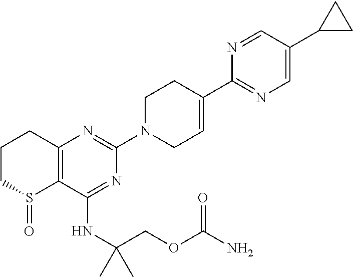 [SMILES: CC(C)(COC(N)=O)Nc1nc(N2CC=C(c3ncc(C4CC4)cn3)CC2)nc2c1[S@](=O)CCC2]

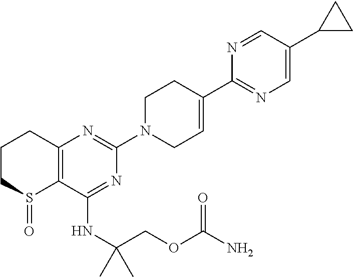 [SMILES: CC(C)(COC(N)=O)Nc1nc(N2CC=C(c3ncc(C4CC4)cn3)CC2)nc2c1[S@@](=O)CCC2]

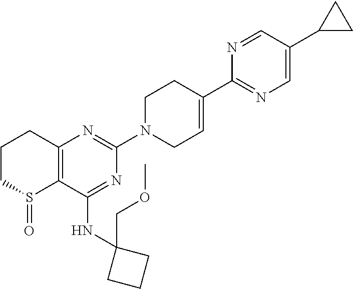 [SMILES: COCC1(Nc2nc(N3CC=C(c4ncc(C5CC5)cn4)CC3)nc3c2[S@](=O)CCC3)CCC1]

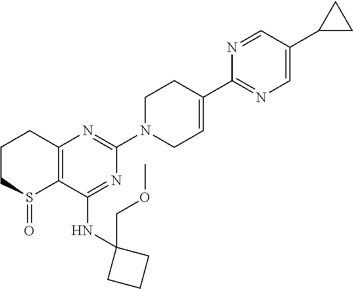 [SMILES: COCC1(Nc2nc(N3CC=C(c4ncc(C5CC5)cn4)CC3)nc3c2[S@@](=O)CCC3)CCC1]

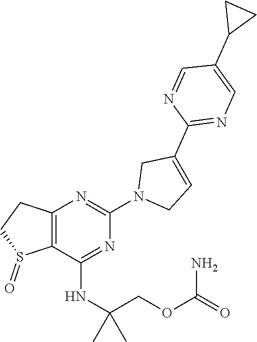 [SMILES: CC(C)(COC(N)=O)Nc1nc(N2CC=C(c3ncc(C4CC4)cn3)C2)nc2c1[S@](=O)CC2]

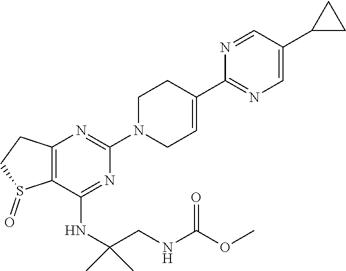 [SMILES: COC(=O)NCC(C)(C)Nc1nc(N2CC=C(c3ncc(C4CC4)cn3)CC2)nc2c1[S@](=O)CC2]

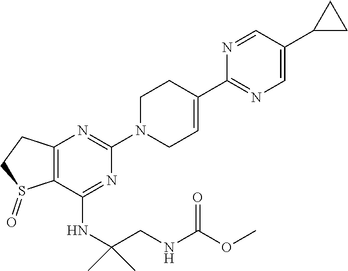 [SMILES: COC(=O)NCC(C)(C)Nc1nc(N2CC=C(c3ncc(C4CC4)cn3)CC2)nc2c1[S@@](=O)CC2]

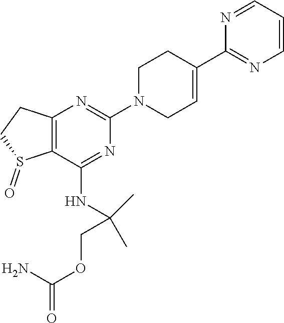 [SMILES: CC(C)(COC(N)=O)Nc1nc(N2CC=C(c3ncccn3)CC2)nc2c1[S@](=O)CC2]

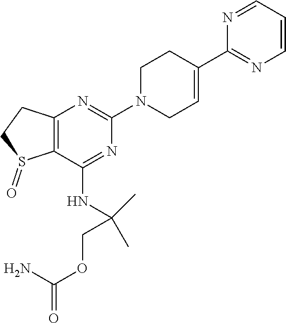 [SMILES: CC(C)(COC(N)=O)Nc1nc(N2CC=C(c3ncccn3)CC2)nc2c1[S@@](=O)CC2]

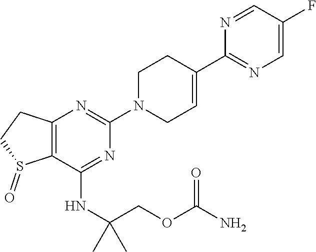 [SMILES: CC(C)(COC(N)=O)Nc1nc(N2CC=C(c3ncc(F)cn3)CC2)nc2c1[S@](=O)CC2]

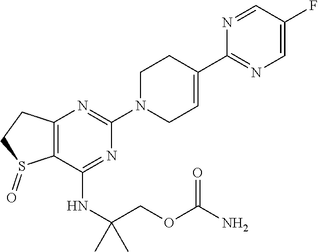 [SMILES: CC(C)(COC(N)=O)Nc1nc(N2CC=C(c3ncc(F)cn3)CC2)nc2c1[S@@](=O)CC2]

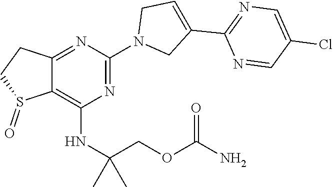 [SMILES: CC(C)(COC(N)=O)Nc1nc(N2CC=C(c3ncc(Cl)cn3)C2)nc2c1[S@](=O)CC2]

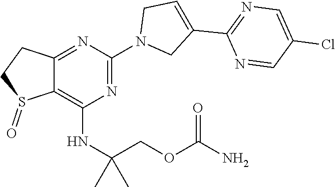 [SMILES: CC(C)(COC(N)=O)Nc1nc(N2CC=C(c3ncc(Cl)cn3)C2)nc2c1[S@@](=O)CC2]

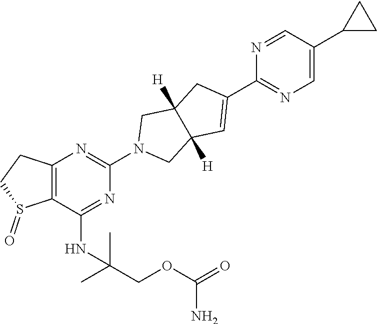 [SMILES: CC(C)(COC(N)=O)Nc1nc(N2C[C@@H]3CC(c4ncc(C5CC5)cn4)=C[C@@H]3C2)nc2c1[S@](=O)CC2]

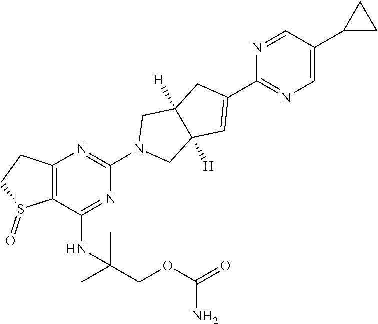 [SMILES: CC(C)(COC(N)=O)Nc1nc(N2C[C@H]3CC(c4ncc(C5CC5)cn4)=C[C@H]3C2)nc2c1[S@](=O)CC2]

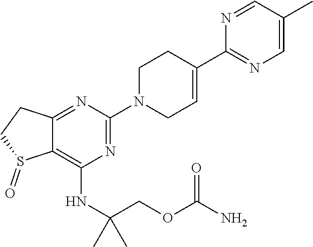 [SMILES: Cc1cnc(C2=CCN(c3nc4c(c(NC(C)(C)COC(N)=O)n3)[S@](=O)CC4)CC2)nc1]

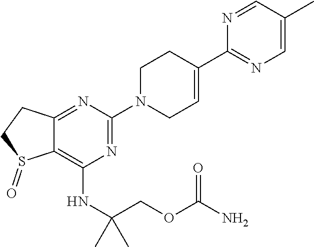 [SMILES: Cc1cnc(C2=CCN(c3nc4c(c(NC(C)(C)COC(N)=O)n3)[S@@](=O)CC4)CC2)nc1]

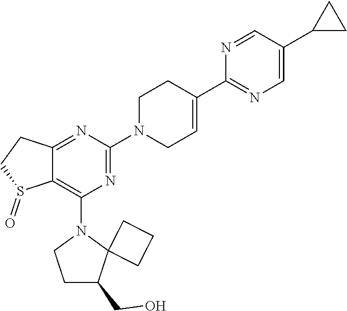 [SMILES: O=[S@@]1CCc2nc(N3CC=C(c4ncc(C5CC5)cn4)CC3)nc(N3CC[C@H](CO)C34CCC4)c21]

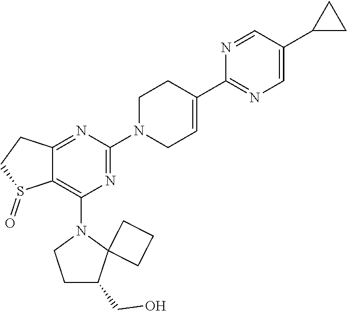 [SMILES: O=[S@@]1CCc2nc(N3CC=C(c4ncc(C5CC5)cn4)CC3)nc(N3CC[C@@H](CO)C34CCC4)c21]

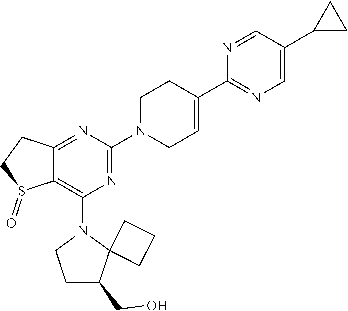 [SMILES: O=[S@]1CCc2nc(N3CC=C(c4ncc(C5CC5)cn4)CC3)nc(N3CC[C@H](CO)C34CCC4)c21]

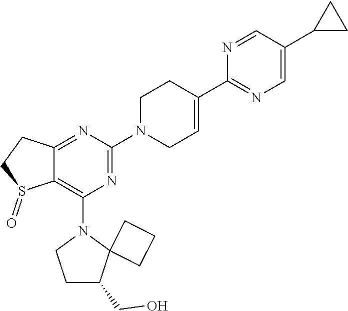 [SMILES: O=[S@]1CCc2nc(N3CC=C(c4ncc(C5CC5)cn4)CC3)nc(N3CC[C@@H](CO)C34CCC4)c21]

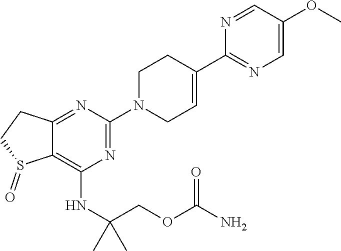 [SMILES: COc1cnc(C2=CCN(c3nc4c(c(NC(C)(C)COC(N)=O)n3)[S@](=O)CC4)CC2)nc1]

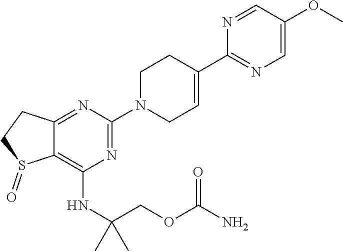 [SMILES: COc1cnc(C2=CCN(c3nc4c(c(NC(C)(C)COC(N)=O)n3)[S@@](=O)CC4)CC2)nc1]

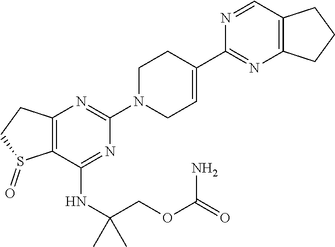 [SMILES: CC(C)(COC(N)=O)Nc1nc(N2CC=C(c3ncc4c(n3)CCC4)CC2)nc2c1[S@](=O)CC2]

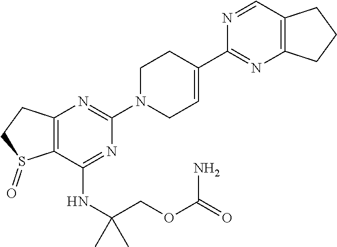 [SMILES: CC(C)(COC(N)=O)Nc1nc(N2CC=C(c3ncc4c(n3)CCC4)CC2)nc2c1[S@@](=O)CC2]

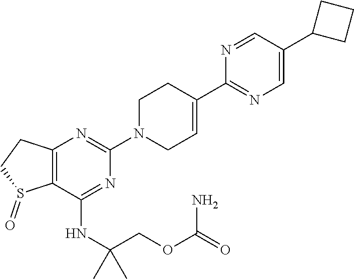 [SMILES: CC(C)(COC(N)=O)Nc1nc(N2CC=C(c3ncc(C4CCC4)cn3)CC2)nc2c1[S@](=O)CC2]

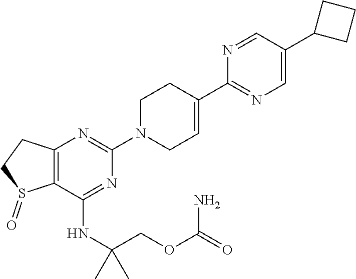 [SMILES: CC(C)(COC(N)=O)Nc1nc(N2CC=C(c3ncc(C4CCC4)cn3)CC2)nc2c1[S@@](=O)CC2]

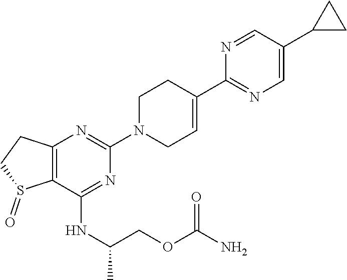 [SMILES: C[C@@H](COC(N)=O)Nc1nc(N2CC=C(c3ncc(C4CC4)cn3)CC2)nc2c1[S@](=O)CC2]

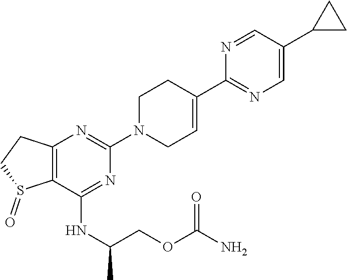 [SMILES: C[C@H](COC(N)=O)Nc1nc(N2CC=C(c3ncc(C4CC4)cn3)CC2)nc2c1[S@](=O)CC2]

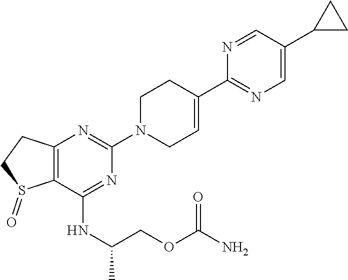 [SMILES: C[C@@H](COC(N)=O)Nc1nc(N2CC=C(c3ncc(C4CC4)cn3)CC2)nc2c1[S@@](=O)CC2]

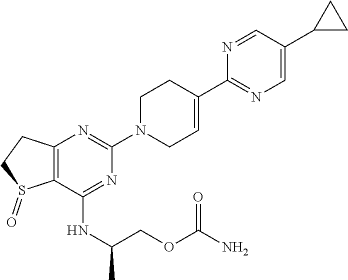 [SMILES: C[C@H](COC(N)=O)Nc1nc(N2CC=C(c3ncc(C4CC4)cn3)CC2)nc2c1[S@@](=O)CC2]

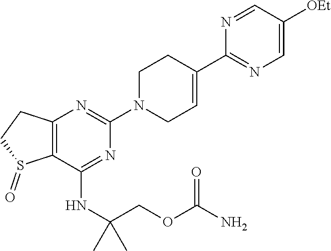 [SMILES: CCOc1cnc(C2=CCN(c3nc4c(c(NC(C)(C)COC(N)=O)n3)[S@](=O)CC4)CC2)nc1]

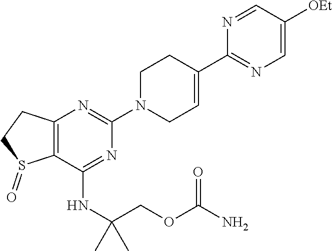 [SMILES: CCOc1cnc(C2=CCN(c3nc4c(c(NC(C)(C)COC(N)=O)n3)[S@@](=O)CC4)CC2)nc1]

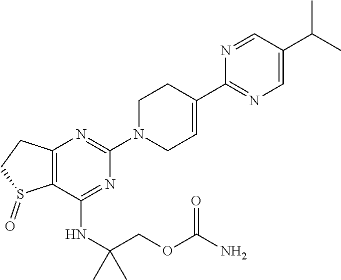 [SMILES: CC(C)c1cnc(C2=CCN(c3nc4c(c(NC(C)(C)COC(N)=O)n3)[S@](=O)CC4)CC2)nc1]

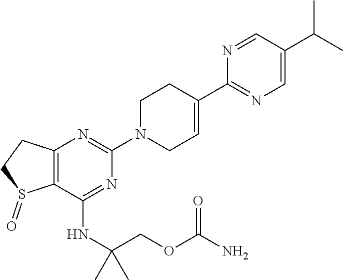 [SMILES: CC(C)c1cnc(C2=CCN(c3nc4c(c(NC(C)(C)COC(N)=O)n3)[S@@](=O)CC4)CC2)nc1]

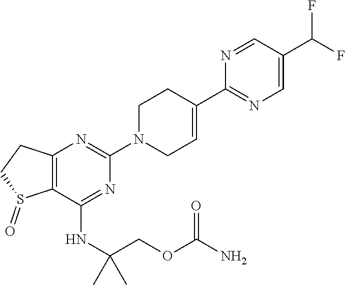 [SMILES: CC(C)(COC(N)=O)Nc1nc(N2CC=C(c3ncc(C(F)F)cn3)CC2)nc2c1[S@](=O)CC2]

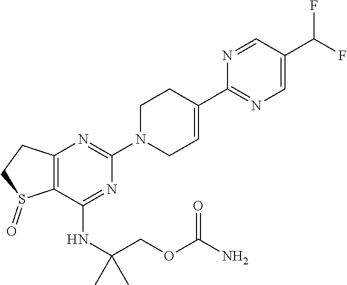 [SMILES: CC(C)(COC(N)=O)Nc1nc(N2CC=C(c3ncc(C(F)F)cn3)CC2)nc2c1[S@@](=O)CC2]

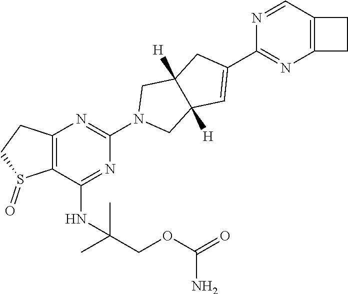 [SMILES: CC(C)(COC(N)=O)Nc1nc(N2C[C@@H]3CC(c4ncc5c(n4)CC5)=C[C@@H]3C2)nc2c1[S@](=O)CC2]

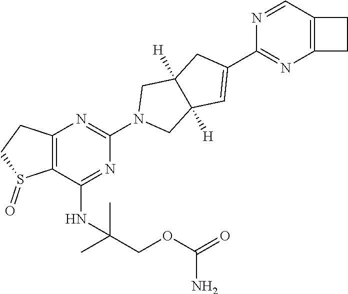 [SMILES: CC(C)(COC(N)=O)Nc1nc(N2C[C@H]3CC(c4ncc5c(n4)CC5)=C[C@H]3C2)nc2c1[S@](=O)CC2]

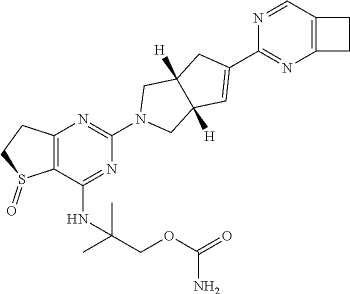 [SMILES: CC(C)(COC(N)=O)Nc1nc(N2C[C@@H]3CC(c4ncc5c(n4)CC5)=C[C@@H]3C2)nc2c1[S@@](=O)CC2]

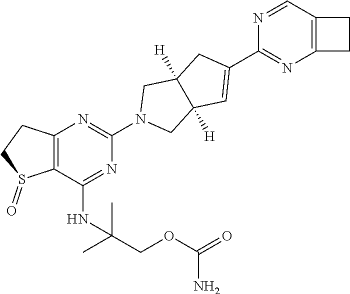 [SMILES: CC(C)(COC(N)=O)Nc1nc(N2C[C@H]3CC(c4ncc5c(n4)CC5)=C[C@H]3C2)nc2c1[S@@](=O)CC2]

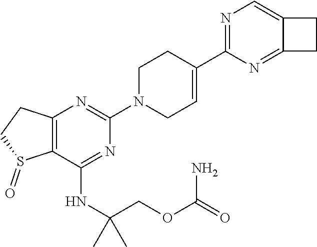 [SMILES: CC(C)(COC(N)=O)Nc1nc(N2CC=C(c3ncc4c(n3)CC4)CC2)nc2c1[S@](=O)CC2]

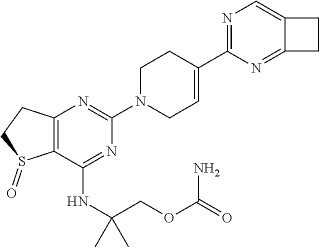 [SMILES: CC(C)(COC(N)=O)Nc1nc(N2CC=C(c3ncc4c(n3)CC4)CC2)nc2c1[S@@](=O)CC2]

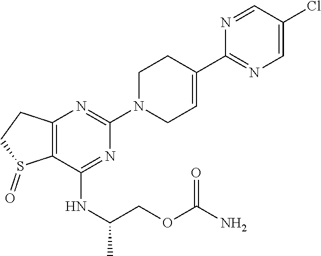 [SMILES: C[C@@H](COC(N)=O)Nc1nc(N2CC=C(c3ncc(Cl)cn3)CC2)nc2c1[S@](=O)CC2]

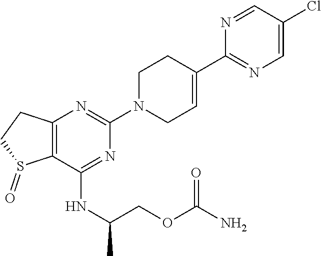 [SMILES: C[C@H](COC(N)=O)Nc1nc(N2CC=C(c3ncc(Cl)cn3)CC2)nc2c1[S@](=O)CC2]

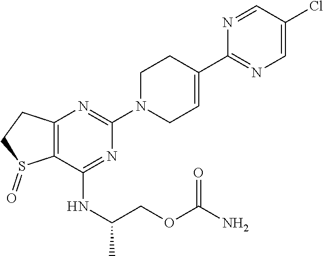 [SMILES: C[C@@H](COC(N)=O)Nc1nc(N2CC=C(c3ncc(Cl)cn3)CC2)nc2c1[S@@](=O)CC2]

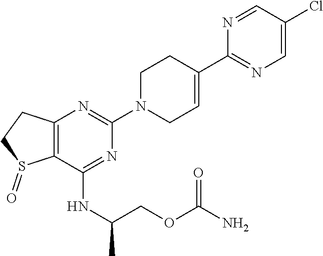 [SMILES: C[C@H](COC(N)=O)Nc1nc(N2CC=C(c3ncc(Cl)cn3)CC2)nc2c1[S@@](=O)CC2]

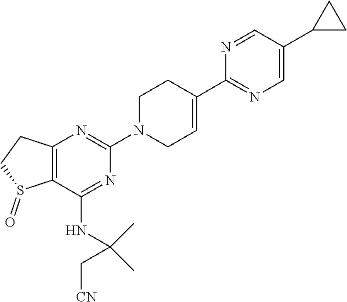 [SMILES: CC(C)(CC#N)Nc1nc(N2CC=C(c3ncc(C4CC4)cn3)CC2)nc2c1[S@](=O)CC2]

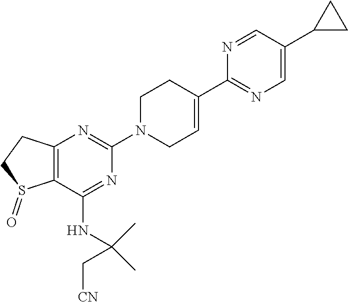 [SMILES: CC(C)(CC#N)Nc1nc(N2CC=C(c3ncc(C4CC4)cn3)CC2)nc2c1[S@@](=O)CC2]

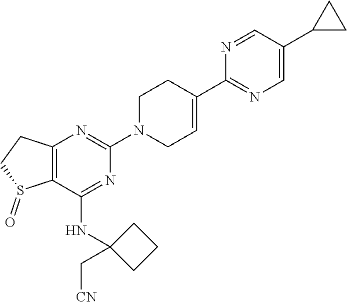 [SMILES: N#CCC1(Nc2nc(N3CC=C(c4ncc(C5CC5)cn4)CC3)nc3c2[S@](=O)CC3)CCC1]

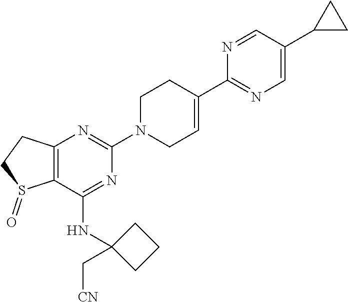 [SMILES: N#CCC1(Nc2nc(N3CC=C(c4ncc(C5CC5)cn4)CC3)nc3c2[S@@](=O)CC3)CCC1]

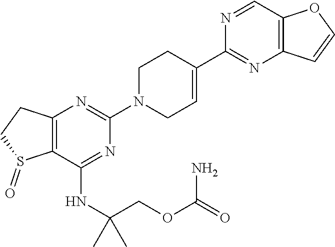 [SMILES: CC(C)(COC(N)=O)Nc1nc(N2CC=C(c3ncc4occc4n3)CC2)nc2c1[S@](=O)CC2]

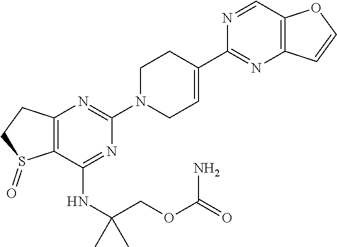 [SMILES: CC(C)(COC(N)=O)Nc1nc(N2CC=C(c3ncc4occc4n3)CC2)nc2c1[S@@](=O)CC2]

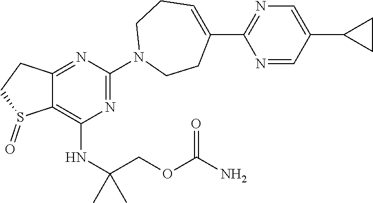 [SMILES: CC(C)(COC(N)=O)Nc1nc(N2CCC=C(c3ncc(C4CC4)cn3)CC2)nc2c1[S@](=O)CC2]

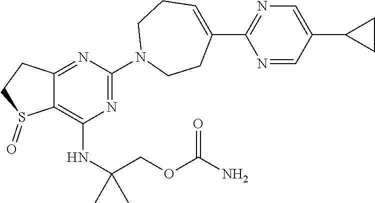 [SMILES: CC(C)(COC(N)=O)Nc1nc(N2CCC=C(c3ncc(C4CC4)cn3)CC2)nc2c1[S@@](=O)CC2]

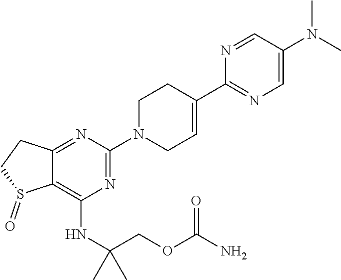 [SMILES: CN(C)c1cnc(C2=CCN(c3nc4c(c(NC(C)(C)COC(N)=O)n3)[S@](=O)CC4)CC2)nc1]

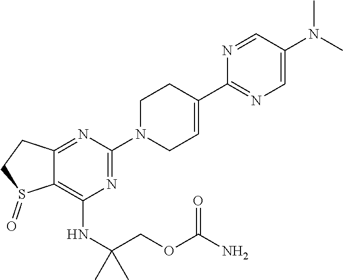 [SMILES: CN(C)c1cnc(C2=CCN(c3nc4c(c(NC(C)(C)COC(N)=O)n3)[S@@](=O)CC4)CC2)nc1]

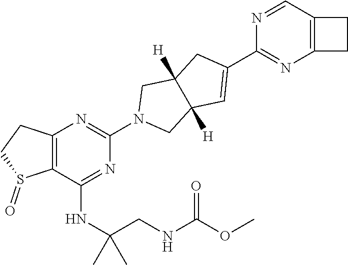 [SMILES: COC(=O)NCC(C)(C)Nc1nc(N2C[C@@H]3CC(c4ncc5c(n4)CC5)=C[C@@H]3C2)nc2c1[S@](=O)CC2]

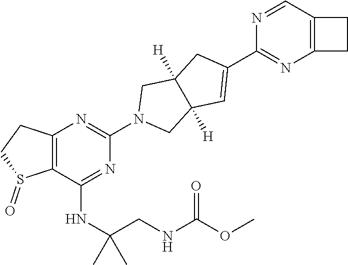 [SMILES: COC(=O)NCC(C)(C)Nc1nc(N2C[C@H]3CC(c4ncc5c(n4)CC5)=C[C@H]3C2)nc2c1[S@](=O)CC2]

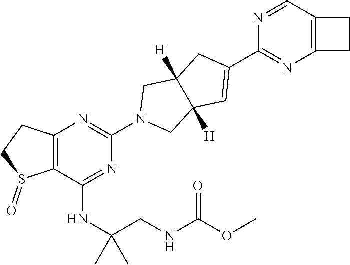 [SMILES: COC(=O)NCC(C)(C)Nc1nc(N2C[C@@H]3CC(c4ncc5c(n4)CC5)=C[C@@H]3C2)nc2c1[S@@](=O)CC2]

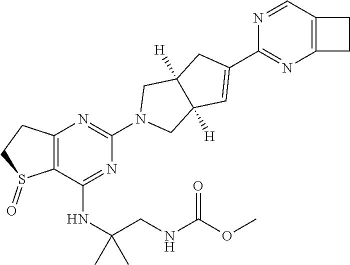 [SMILES: COC(=O)NCC(C)(C)Nc1nc(N2C[C@H]3CC(c4ncc5c(n4)CC5)=C[C@H]3C2)nc2c1[S@@](=O)CC2]

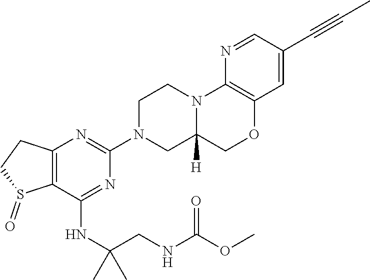 [SMILES: CC#Cc1cnc2c(c1)OC[C@@H]1CN(c3nc4c(c(NC(C)(C)CNC(=O)OC)n3)[S@](=O)CC4)CCN21]

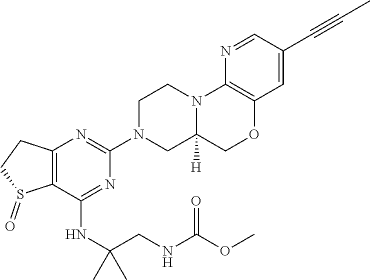 [SMILES: CC#Cc1cnc2c(c1)OC[C@H]1CN(c3nc4c(c(NC(C)(C)CNC(=O)OC)n3)[S@](=O)CC4)CCN21]

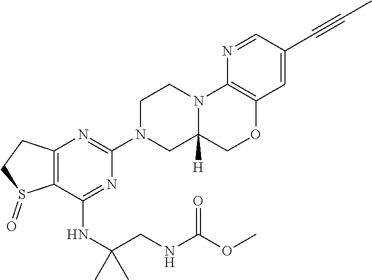 [SMILES: CC#Cc1cnc2c(c1)OC[C@@H]1CN(c3nc4c(c(NC(C)(C)CNC(=O)OC)n3)[S@@](=O)CC4)CCN21]

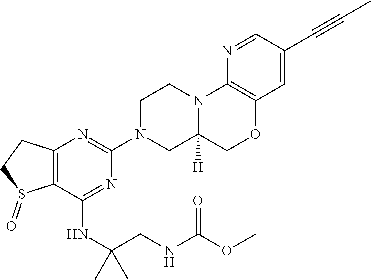 [SMILES: CC#Cc1cnc2c(c1)OC[C@H]1CN(c3nc4c(c(NC(C)(C)CNC(=O)OC)n3)[S@@](=O)CC4)CCN21]

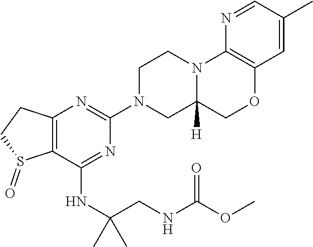 [SMILES: COC(=O)NCC(C)(C)Nc1nc(N2CCN3c4ncc(C)cc4OC[C@@H]3C2)nc2c1[S@](=O)CC2]

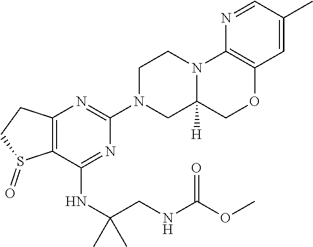 [SMILES: COC(=O)NCC(C)(C)Nc1nc(N2CCN3c4ncc(C)cc4OC[C@H]3C2)nc2c1[S@](=O)CC2]

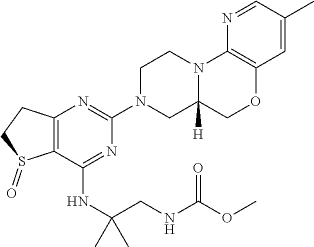 [SMILES: COC(=O)NCC(C)(C)Nc1nc(N2CCN3c4ncc(C)cc4OC[C@@H]3C2)nc2c1[S@@](=O)CC2]

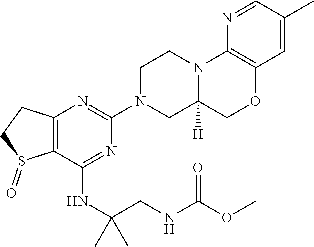 [SMILES: COC(=O)NCC(C)(C)Nc1nc(N2CCN3c4ncc(C)cc4OC[C@H]3C2)nc2c1[S@@](=O)CC2]

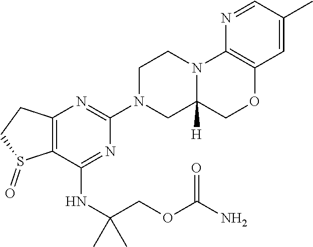 [SMILES: Cc1cnc2c(c1)OC[C@@H]1CN(c3nc4c(c(NC(C)(C)COC(N)=O)n3)[S@](=O)CC4)CCN21]

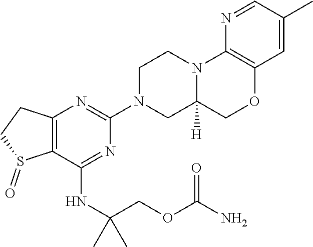 [SMILES: Cc1cnc2c(c1)OC[C@H]1CN(c3nc4c(c(NC(C)(C)COC(N)=O)n3)[S@](=O)CC4)CCN21]

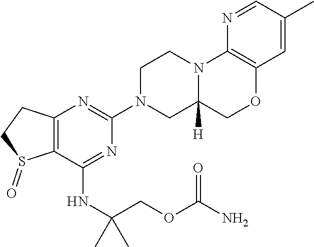 [SMILES: Cc1cnc2c(c1)OC[C@@H]1CN(c3nc4c(c(NC(C)(C)COC(N)=O)n3)[S@@](=O)CC4)CCN21]

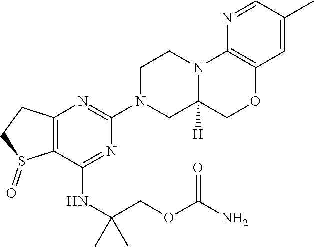 [SMILES: Cc1cnc2c(c1)OC[C@H]1CN(c3nc4c(c(NC(C)(C)COC(N)=O)n3)[S@@](=O)CC4)CCN21]

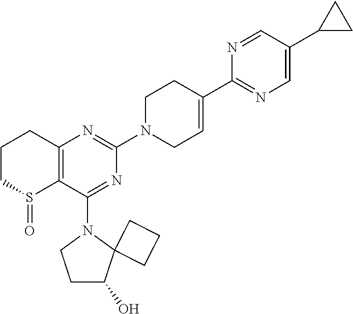 [SMILES: O=[S@@]1CCCc2nc(N3CC=C(c4ncc(C5CC5)cn4)CC3)nc(N3CC[C@@H](O)C34CCC4)c21]

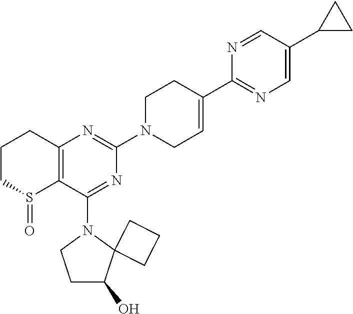 [SMILES: O=[S@@]1CCCc2nc(N3CC=C(c4ncc(C5CC5)cn4)CC3)nc(N3CC[C@H](O)C34CCC4)c21]

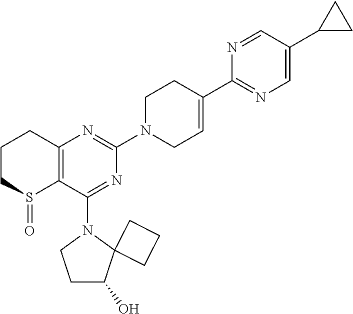 [SMILES: O=[S@]1CCCc2nc(N3CC=C(c4ncc(C5CC5)cn4)CC3)nc(N3CC[C@@H](O)C34CCC4)c21]

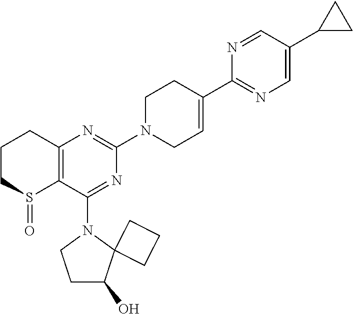 [SMILES: O=[S@]1CCCc2nc(N3CC=C(c4ncc(C5CC5)cn4)CC3)nc(N3CC[C@H](O)C34CCC4)c21]

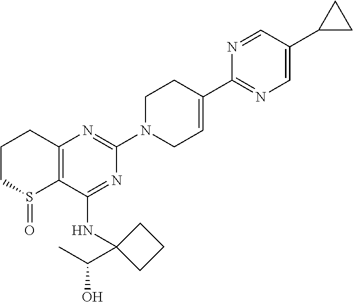 [SMILES: C[C@@H](O)C1(Nc2nc(N3CC=C(c4ncc(C5CC5)cn4)CC3)nc3c2[S@](=O)CCC3)CCC1]

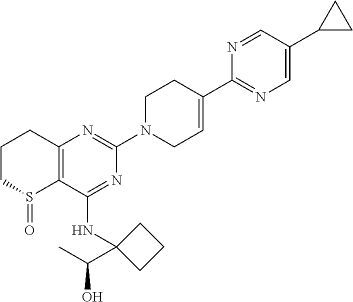 [SMILES: C[C@H](O)C1(Nc2nc(N3CC=C(c4ncc(C5CC5)cn4)CC3)nc3c2[S@](=O)CCC3)CCC1]

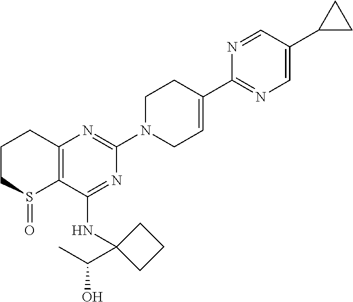 [SMILES: C[C@@H](O)C1(Nc2nc(N3CC=C(c4ncc(C5CC5)cn4)CC3)nc3c2[S@@](=O)CCC3)CCC1]

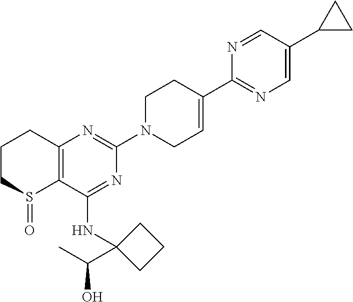 [SMILES: C[C@H](O)C1(Nc2nc(N3CC=C(c4ncc(C5CC5)cn4)CC3)nc3c2[S@@](=O)CCC3)CCC1]

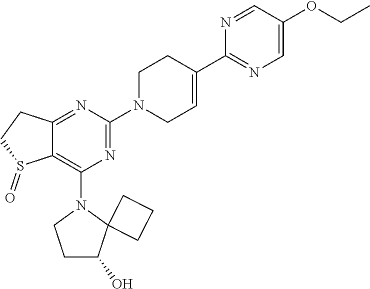 [SMILES: CCOc1cnc(C2=CCN(c3nc4c(c(N5CC[C@@H](O)C56CCC6)n3)[S@](=O)CC4)CC2)nc1]

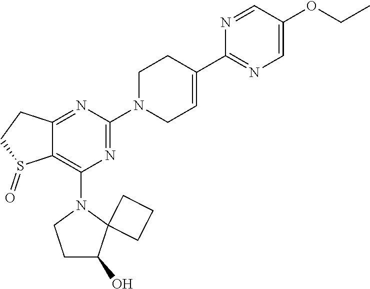 [SMILES: CCOc1cnc(C2=CCN(c3nc4c(c(N5CC[C@H](O)C56CCC6)n3)[S@](=O)CC4)CC2)nc1]

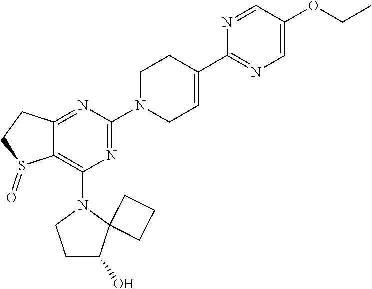 [SMILES: CCOc1cnc(C2=CCN(c3nc4c(c(N5CC[C@@H](O)C56CCC6)n3)[S@@](=O)CC4)CC2)nc1]

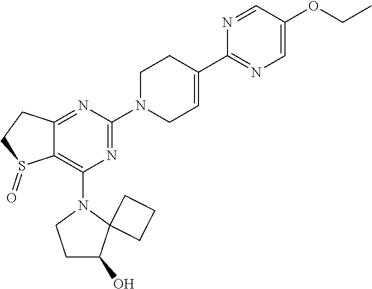 [SMILES: CCOc1cnc(C2=CCN(c3nc4c(c(N5CC[C@H](O)C56CCC6)n3)[S@@](=O)CC4)CC2)nc1]

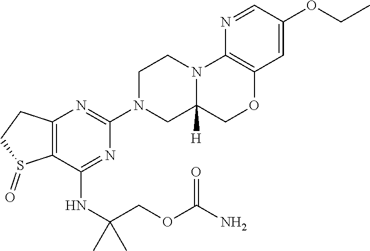 [SMILES: CCOc1cnc2c(c1)OC[C@@H]1CN(c3nc4c(c(NC(C)(C)COC(N)=O)n3)[S@](=O)CC4)CCN21]

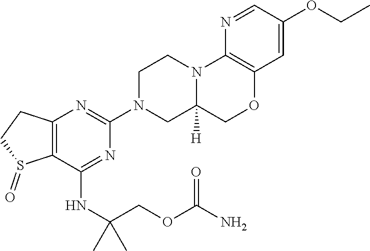 [SMILES: CCOc1cnc2c(c1)OC[C@H]1CN(c3nc4c(c(NC(C)(C)COC(N)=O)n3)[S@](=O)CC4)CCN21]

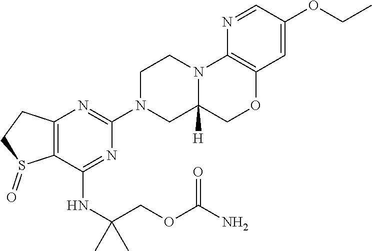 [SMILES: CCOc1cnc2c(c1)OC[C@@H]1CN(c3nc4c(c(NC(C)(C)COC(N)=O)n3)[S@@](=O)CC4)CCN21]

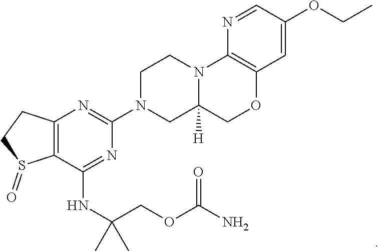 [SMILES: CCOc1cnc2c(c1)OC[C@H]1CN(c3nc4c(c(NC(C)(C)COC(N)=O)n3)[S@@](=O)CC4)CCN21]

“abs” denotes absolute configuration.

The present disclosure further provides a composition or pharmaceutical preparation comprising the compound or the stereoisomer or pharmaceutically acceptable salt thereof according to any one of the preceding solutions, and a pharmaceutically acceptable carrier and/or auxiliary material. The pharmaceutical composition can be in a unit preparation form (the unit preparation is also referred to as “preparation specification”).

Furthermore, the composition or pharmaceutical preparation of the present disclosure comprises 1-1500 mg of the compound or the stereoisomer or pharmaceutically acceptable salt thereof according to any one of the preceding solutions, and a pharmaceutically acceptable carrier and/or auxiliary material.

The present disclosure further provides the use of the compound or the stereoisomer or pharmaceutically acceptable salt thereof according to any one of the preceding solutions in the preparation of a drug for treating/preventing a PDE4B-mediated disease. Furthermore, the PDE4B-mediated disease is a cancer, COPD, idiopathic pulmonary fibrosis, or an interstitial lung disease.

The present disclosure further provides a method for treating a disease in a mammal or human, comprising administering to a subject a therapeutically effective amount of the compound or the stereoisomer or pharmaceutically acceptable salt thereof as shown in any one of the preceding solutions, wherein the disease is preferably a cancer, COPD, idiopathic pulmonary fibrosis, or an interstitial lung disease, and the therapeutically effective amount is preferably 1-1500 mg. In some embodiments, the mammal described in the present disclosure does not include human.

The “effective amount” or “therapeutically effective amount” as described in the present application refers to administration of a sufficient amount of the compound disclosed in the present application that will alleviate to some extent one or more symptoms of the diseases or conditions being treated. In some embodiments, the outcome is the reduction and/or remission of signs, symptoms, or causes of the disease, or any other desired change in the biological system. For example, an “effective amount” in terms of the therapeutic use is an amount comprising the compound disclosed in the present application that is required to provide clinically significant reduction of the symptoms of the disease. Examples of the therapeutically effective amount include, but are not limited to 1-1500 mg, 1-1400 mg, 1-1300 mg, 1-1200 mg, 1-1000 mg, 1-900 mg, 1-800 mg, 1-700 mg, 1-600 mg, 1-500 mg, 1-400 mg, 1-300 mg, 1-250 mg, 1-200 mg, 1-150 mg, 1-125 mg, 1-100 mg, 1-80 mg, 1-60 mg, 1-50 mg, 1-40 mg, 1-25 mg, 1-20 mg, 5-1500 mg, 5-1000 mg, 5-900 mg, 5-800 mg, 5-700 mg, 5-600 mg, 5-500 mg, 5-400 mg, 5-300 mg, 5-250 mg, 5-200 mg, 5-150 mg, 5-125 mg, 5-100 mg, 5-90 mg, 5-70 mg, 5-80 mg, 5-60 mg, 5-50 mg, 5-40 mg, 5-30 mg, 5-25 mg, 5-20 mg, 10-1500 mg, 10-1000 mg, 10-900 mg, 10-800 mg, 10-700 mg, 10-600 mg, 10-500 mg, 10-450 mg, 10-400 mg, 10-300 mg, 10-250 mg, 10-200 mg, 10-150 mg, 10-125 mg, 10-100 mg, 10-90 mg, 10-80 mg, 10-70 mg, 10-60 mg, 10-50 mg, 10-40 mg, 10-30 mg, 10-20 mg; 20-1500 mg, 20-1000 mg, 20-900 mg, 20-800 mg, 20-700 mg, 20-600 mg, 20-500 mg, 20-400 mg, 20-350 mg, 20-300 mg, 20-250 mg, 20-200 mg, 20-150 mg, 20-125 mg, 20-100 mg, 20-90 mg, 20-80 mg, 20-70 mg, 20-60 mg, 20-50 mg, 20-40 mg, 20-30 mg; 50-1500 mg, 50-1000 mg, 50-900 mg, 50-800 mg, 50-700 mg, 50-600 mg, 50-500 mg, 50-400 mg, 50-300 mg, 50-250 mg, 50-200 mg, 50-150 mg, 50-125 mg, 50-100 mg; and 100-1500 mg, 100-1000 mg, 100-900 mg, 100-800 mg, 100-700 mg, 100-600 mg, 100-500 mg, 100-400 mg, 100-300 mg, 100-250 mg, and 100-200 mg.

In some embodiments, the pharmaceutical composition or preparation of the present disclosure contains the above-mentioned therapeutically effective amount of the compound, or the stereoisomer, solvate, or pharmaceutically acceptable salt thereof according to the present disclosure.

The present disclosure relates to a pharmaceutical composition or pharmaceutical preparation comprising a therapeutically effective amount of the compound or the stereoisomer or pharmaceutically acceptable salt thereof according to the present disclosure, and a carrier and/or auxiliary material. The pharmaceutical composition can be in a unit preparation form (the amount of the active drug in the unit preparation is also referred to as “preparation specification”). In some embodiments, the pharmaceutical composition comprises, without limitation, 1 mg, 1.25 mg, 2.5 mg, 5 mg, 10 mg, 12.5 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 110 mg, 120 mg, 125 mg, 130 mg, 140 mg, 150 mg, 160 mg, 170 mg, 180 mg, 190 mg, 200 mg, 210 mg, 220 mg, 230 mg, 240 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 850 mg, 900 mg, 950 mg, 1000 mg, 1100 mg, 1200 mg, 1300 mg, 1400 mg, or 1500 mg of the compound or the stereoisomer or pharmaceutically acceptable salt thereof according to the present disclosure.

A method for treating a disease in a mammal is provided, which comprises administering to a subject a therapeutically effective amount of the compound or the stereoisomer or pharmaceutically acceptable salt thereof according to the present disclosure, and a pharmaceutically acceptable carrier and/or auxiliary material, wherein the therapeutically effective amount is preferably 1-1500 mg, and the disease is preferably a cancer, COPD, idiopathic pulmonary fibrosis, or an interstitial lung disease.

A method for treating a disease in a mammal or human is provided, which comprises administering to a subject the compound or the stereoisomer or pharmaceutically acceptable salt thereof according to the present disclosure, as a drug, and a pharmaceutically acceptable carrier and/or auxiliary material at a daily dose of 1-1500 mg/day, wherein the daily dose may be a single dose or divided doses. In some embodiments, the daily dose includes, but is not limited to, 10-1500 mg/day, 20-1500 mg/day, 25-1500 mg/day, 50-1500 mg/day, 75-1500 mg/day, 100-1500 mg/day, 200-1500 mg/day, 10-1000 mg/day, 20-1000 mg/day, 25-1000 mg/day, 50-1000 mg/day, 75-1000 mg/day, 100-1000 mg/day, 200-1000 mg/day, 25-800 mg/day, 50-800 mg/day, 100-800 mg/day, 200-800 mg/day, 25-400 mg/day, 50-400 mg/day, 100-400 mg/day, and 200-400 mg/day. In some embodiments, the daily dose includes but is not limited to 1 mg/day, 5 mg/day, 10 mg/day, 20 mg/day, 25 mg/day, 50 mg/day, 75 mg/day, 100 mg/day, 125 mg/day, 150 mg/day, 200 mg/day, 400 mg/day, 600 mg/day, 800 mg/day, 1000 mg/day, 1200 mg/day, 1400 mg/day, and 1500 mg/day.

The present disclosure relates to a kit, which may comprise a composition in a single-dose or multi-dose form, wherein the kit comprises the compound or the stereoisomer or pharmaceutically acceptable salt thereof according to the present disclosure, and the amount of the compound or the stereoisomer or pharmaceutically acceptable salt thereof according to the present disclosure is the same as that in the above-mentioned pharmaceutical composition.

In the present disclosure, the amount of the compound or the stereoisomer or pharmaceutically acceptable salt thereof according to the present disclosure is calculated in the form of a free base in each case.

The term “preparation specification” refers to the weight of the active drug contained in each vial, tablet, or other unit preparation.

### Synthesis Route

Methods for preparing PDE4B inhibitors are introduced in patent documents such as WO 2013026797 A1. Those skilled in the art would prepare the compound of the present disclosure in conjunction with this document with a known organic synthesis technique using commercially available chemicals and (or) compounds described in chemical documents as starting materials. “Commercially available chemicals” are obtained from regular commercial sources, and suppliers include: Titan Technology Co., Ltd., Energy Chemical Co., Ltd., Shanghai Demo Co., Ltd., Chengdu Kelong Chemical Co., Ltd., Accela ChemBio Co., Ltd., PharmaBlock Sciences (Nanjing), Inc., WuXi Apptec Co., Ltd., J&K Scientific Co., Ltd., etc.

Specific and similar reactants can be selectively identified by the indexes of known chemicals prepared by the Chemical Abstracts Service of the American Chemical Society, wherein the indexes are available in most public libraries and university libraries and online. Chemicals that are known but not commercially available in the catalog are optionally prepared by custom chemical synthesis plants, wherein many of standard chemical supply plants (for example, those listed above) provide custom synthesis services.

### Terminology

Unless otherwise specially specified, the terms in the present disclosure have the following meanings.

The term “halogen” herein refers to F, Cl, Br, I, or isotopes thereof.

The term “halo” or “substituted with halogen” means that a hydrogen atom is replaced with one or more groups selected from F, Cl, Br, I, or isotopes thereof, wherein the upper limit of the number of halogen substituents is equal to the sum of the number of hydrogens that can be replaced in the group to be substituted. Without particular limitation, the number of halogen substituents is any integer between 1 and the upper limit, and when the number of halogen substituents is greater than 1, the group to be substituted can be substituted with the same or different halogen.

The term “deuterated” or “deuterated product” refers to the case where a hydrogen atom on alkyl, cycloalkyl, alkylene, aryl, heteroaryl, mercapto, heterocycloalkyl, alkenyl, alkynyl and other groups is replaced with at least one isotope deuterium, wherein the upper limit of the number of deuterium substituents is equal to the sum of the number of hydrogens that can be replaced in the group to be substituted. Without particular limitation, the number of deuterium substituents is any integer between 1 and the upper limit, preferably 1-20 deuterium atoms, more preferably 1-10 deuterium atoms, more preferably 1-6 deuterium atoms, and further preferably 1-3 deuterium atoms.

The term “alkyl” refers to a monovalent linear or branched saturated aliphatic hydrocarbon group. Unless otherwise specially specified, the alkyl refers to an alkyl group comprising 1 to 20 carbon atoms, preferably an alkyl group comprising 1 to 8 carbon atoms, more preferably an alkyl group comprising 1 to 6 carbon atoms, further preferably an alkyl group comprising 1 to 4 carbon atoms, and further preferably an alkyl group comprising 1-2 carbon atoms. Non-limiting examples include methyl, ethyl, n-propyl, isopropyl, n-butyl, sec-butyl, neobutyl, tert-butyl, n-pentyl, isoamyl, neopentyl, n-hexyl, and various branched isomers thereof.

The term “alkylene” refers to a divalent linear or branched chain divalent saturated alkyl, and examples of alkylene include, but are not limited to, methylene, ethylene, propylene, butylene, etc.

The term “cycloalkyl” refers to a monovalent non-aromatic, partially unsaturated or fully saturated, substituted or unsubstituted carbocyclic hydrocarbon group, which generally has 3 to 12 carbon atoms, preferably 3-10 carbon atoms, more preferably 3-6 carbon atoms, and further preferably 3-4 carbon atoms, unless otherwise specially specified. Non-limiting examples include cyclopropyl, cyclobutyl, cyclopentyl, cyclohexyl,

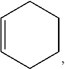

cycloheptyl, etc.

The term “cycloalkylene” refers to a divalent group of “cycloalkyl”, and non-limiting examples include cyclopropylene, cyclobutylene, etc.

The term “heterocycle” or “heterocyclyl” refers to a substituted or unsubstituted, saturated or unsaturated, aromatic or non-aromatic ring comprising 1 to 3 heteroatoms selected from N, O, or S unless otherwise specifically defined, including monocyclic heterocycles, bridged bicyclic heterocycles, fused bicyclic heterocycles, spiro bicyclic heterocycles, etc., which are 3- to 12-membered heterocycles, more preferably 4- to 12-membered heterocycles, more preferably 4- to 10-membered heterocycles, and further preferably 4- to 7-membered heterocycles unless otherwise specifically defined. The definition thereof comprises heterocycloalkyl and heteroaryl. The N and S in the heterocyclyl ring may be oxidized into various oxidation states. Heterocyclyl can be connected to a heteroatom or a carbon atom, and non-limiting examples of heterocyclyl include oxiranyl, aziridinyl, oxetanyl, azetidinyl, 1,3-dioxolanyl, 1,4-dioxolanyl, 1,3-dioxanyl, azepanyl, pyridinyl, furyl, thienyl, pyranyl, N-alkylpyrrolyl, pyrimidinyl, pyrazinyl, pyrazolyl, pyridazinyl, imidazolyl, piperidinyl, piperidinyl, morpholinyl, thiomorpholinyl, 1,3-dithianyl, dihydrofuryl, dihydropyranyl, dithiolanyl, tetrahydrofuryl, tetrahydropyrrolyl, tetrahydroimidazolyl, oxazolyl, dihydrooxazolyl, tetrahydrooxazolyl, tetrahydrothiazolyl, tetrahydropyranyl, benzimidazolyl, benzopyridinyl, pyrrolopyridinyl, benzodihydrofuryl, azabicyclo[3.2.1]octyl, azabicyclo[5.2.0]nonanyl, oxatricyclo[5.3.1.1]dodecyl, azaadamantyl, oxaspiro[3.3]heptyl,

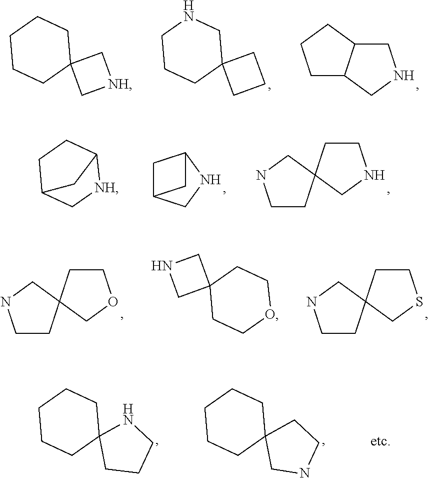

The term “heterocyclylene” is a divalent group corresponding to “heterocyclyl”, and non-limiting examples include imidazolylene, piperidinylene, aziridinylene, etc.

The term “carbocyclic” or “carbocyclyl” refers to a substituted or unsubstituted, saturated or unsaturated, aromatic or non-aromatic carbocyclic group, including monocyclic carbocyclic rings, bridged bicyclic rings, fused bicyclic rings, spiro bicyclic rings, etc., which have 3 to 12 carbon atoms, preferably 3-10 carbon atoms, and further preferably 3-6 carbon atoms unless otherwise specially specified. The definition thereof comprises cycloalkyl and aryl. In non-limiting embodiments, monocyclic carbocyclic rings include cyclopropyl, cyclobutyl, cyclopentyl, cyclohexyl, cycloheptyl or phenyl,

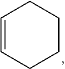

etc., bridged bicyclic rings include

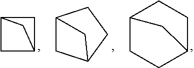

etc., fused bicyclic rings include

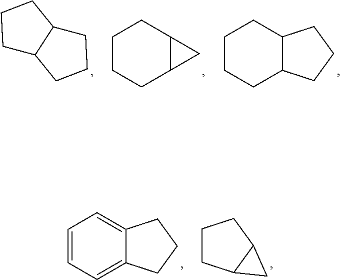

etc., and spiro bicyclic rings include

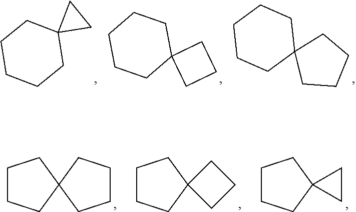

etc.

The term “aryl” refers to an aromatic carbocyclic ring. Non-limiting examples include phenyl, naphthyl, etc.

The term “alkynyl” refers to a linear or branched monovalent unsaturated hydrocarbon group containing one or more carbon-carbon triple bonds. Unless otherwise specially specified, the alkynyl contains 2-6 carbon atoms, preferably 2-4 carbon atoms, and non-limiting examples include ethynyl, propynyl, propargyl, etc.

The term “alkenyl” refers to a linear or branched monovalent unsaturated hydrocarbon group containing one or more carbon-carbon double bonds. Unless otherwise specially specified, the alkenyl contains 2-6 carbon atoms, preferably contains 2-4 carbon atoms, and non-limiting examples are ethenyl, propenyl, allyl, 2-butenyl, 1-butenyl, etc.

The term “alkoxy” or “alkyloxy” refers to —O-alkyl. Without particular limitation, alkoxy or alkyloxy is —O—C₁₋₈ alkyl, preferably —O—C₁₋₆ alkyl, more preferably —O—C₁₋₄ alkyl, and further preferably —O—C₁₋₂ alkyl. Non-limiting examples include methoxy, ethoxy, n-propoxy, isopropoxy, n-butoxy, secbutoxy, tert-butoxy, n-pentoxy, n-hexyloxy, cyclopropoxy, cyclobutoxy, etc.

The term “haloalkoxy” refers to —O-haloalkyl. Without particular limitation, the haloalkoxy is —O-halo C₁₋₈ alkyl, preferably —O-halo C₁₋₆ alkyl, more preferably —O-halo C₁₋₄ alkyl, and further preferably —O-halo C₁₋₂ alkyl. Non-limiting examples of haloalkoxy include monofluoromethoxy, difluoromethoxy, trifluoromethoxy, difluoroethyloxy, etc.

The term “C₁₋₄ alkylacyl” refers to C₁₋₄ alkyl-C(O)—. Non-limiting examples include formyl, acetyl, and propionyl.

The term “C₁₋₄ alkylsulfonyl” refers to C₁₋₄ alkyl-S(O)₂—. Non-limiting examples include methylsulfonyl, ethylsulfonyl, and propylsulfonyl.

The term “heteroaromatic ring” or “heteroaryl” refers to an aromatic heterocycle. Non-limiting examples include pyrazolyl, pyrimidinyl, thiazolyl, pyridinyl, furyl, etc.

The term “heterocycloalkyl” refers to a non-aromatic, partially unsaturated or fully saturated heterocycle, which generally has 4 to 12 ring members, preferably 4 to 10 ring members, more preferably 4 to 7 ring members, and further preferably 5 or 6 ring members. In addition to carbon atoms, heterocycloalkyl further comprises 1-3 heteroatoms selected from N, S, O, Si, and P as ring members. Non-limiting examples include azetidinyl, morpholinyl, piperazinyl, piperidinyl, tetrahydropyranyl, oxetanyl, etc.

The term “alkylamino” or “alkamino” refers to amino substituted with one or two alkyl, and is also written as —N-(alkyl)₂ or —NH-alkyl, wherein the latter is also known as monoalkylamino. Non-limiting examples include dimethylamino, monomethylamino, diethylamino, monoethylamino, etc.

The term “optional” or “optionally” means that the events or circumstances described subsequently may but not necessarily occur, and the description includes the occasions where the events or circumstances occur or do not occur. For example, “alkyl optionally substituted with F” means that the alkyl may but not necessarily be substituted with F, and the description includes the case where the alkyl is substituted with F and the case where the alkyl is not substituted with F.

The term “pharmaceutically acceptable salt” refers to a salt of the compound of the present disclosure, which salt maintains the biological effectiveness and characteristics of a free acid or a free base and is obtained by reacting the free acid with a non-toxic inorganic base or organic base, or reacting the free base with a non-toxic inorganic acid or organic acid.

The term “pharmaceutical composition” represents a mixture of one or more compounds or stereoisomers, solvates, pharmaceutically acceptable salts or co-crystals thereof as described herein and other components including physiologically/pharmaceutically acceptable carriers and/or excipients.

The term “carrier” refers to: a system that does not cause significant irritation to the organism and does not eliminate the biological activity and characteristics of the administered compound and can change the way the drug enters the human body and the distribution of the drug in the body, control the release rate of the drug and delivery the drug to targeted organs. Non-limiting examples of the carrier include microcapsule, microsphere, nanoparticle, liposome, etc.

The term “excipient” refers to: a substance that is not a therapeutic agent per se, but used as a diluent, auxiliary material, adhesive and/or vehicle for addition to a pharmaceutical composition, thereby improving the disposal or storage properties thereof, or allowing to or promoting the formation of a compound or a pharmaceutical composition into a unit dosage form for administration. As is known to those skilled in the art, pharmaceutically acceptable excipients can provide various functions and can be described as a wetting agent, a buffer, a suspending agent, a lubricant, an emulsifier, a disintegrant, an absorbent, a preservative, a surfactant, a colorant, a flavoring agent, and a sweetening agent. Examples of pharmaceutically acceptable excipients include, but are not limited to: (1) sugars, such as lactose, glucose, and sucrose; (2) starch, such as corn starch and potato starch; (3) cellulose and derivatives thereof, such as sodium carboxymethyl cellulose, ethyl cellulose, cellulose acetate, hydroxypropyl methylcellulose, hydroxypropyl cellulose, microcrystalline cellulose, and croscarmellose (such as croscarmellose sodium); (4) tragacanth powder; (5) malt; (6) gelatin; (7) talc; (8) excipients, such as cocoa butter or suppository wax; (9) oils, such as peanut oil, cottonseed oil, safflower oil, sesame oil, olive oil, corn oil, and soybean oil; (10) diols, such as propylene glycol; (11) polyols, such as glycerol, sorbitol, mannitol, and polyethylene glycol; (12) esters, such as ethyl oleate and ethyl laurate; (13) agar; (14) buffers, such as magnesium hydroxide and aluminum hydroxide; (15) alginic acid; (16) pyrogen-free water; (17) isotonic saline; (18) Ringer's solution; (19) ethanol; (20) pH buffer solution; (21) polyester, polycarbonate and/or polyanhydride; and (22) other non-toxic compatible substances used in a pharmaceutical preparation.

The term “stereoisomer” refers to an isomer produced as a result of different spatial arrangement of atoms in molecules, including cis-trans isomers, enantiomers and conformational isomers.

The term “solvate” refers to a substance formed by the compound of the present disclosure or the salt thereof and a stoichiometric or non-stoichiometric solvent bound via an intermolecular non-covalent force. When the solvent is water, the solvate is a hydrate.

The term “co-crystal” refers to a crystal formed by the combination of active pharmaceutical ingredient (API) and co-crystal former (CCF) under the action of hydrogen bonds or other non-covalent bonds. The pure state of API and CCF are both solid at room temperature, and there is a fixed stoichiometric ratio between various components. The co-crystal is a multi-component crystal, which includes both a binary co-crystal formed between two neutral solids and a multi-element co-crystal formed between a neutral solid and a salt or solvate.

### BRIEF DESCRIPTION OF THE DRAWINGS

FIG. 1 shows that compound 51 inhibits LPS-induced expression of TNF-α in the lung tissue of mice in a dose-dependent manner.

FIG. 2 shows that compound 84 inhibits LPS-induced expression of TNF-α in the lung tissue of mice in a dose-dependent manner.

### DETAILED DESCRIPTION

The content of the present disclosure is described in detail by means of the following examples. In examples in which no specific conditions are indicated, experimental methods are carried out under conventional conditions. The listed examples are intended to better illustrate the content of the present disclosure but should not be construed as limiting the content of the present disclosure. Non-essential improvements and adjustments made to the embodiments by those skilled in the art according to the above Summary of the Invention still fall within the scope of protection of the present disclosure.
Dess-Martin reagent: 1,1,1-Triacetoxy-1,1-dihydro-1,2-benziodoxol-3-(1H)-one
DIPEA: N,N-diisopropylethylamine
NMP: N-methylpyrrolidone
TBDMSCl: Tert-butyl dimethylsilyl chloride
DMAP: 4-Dimethylaminopyridine
HATU: 2-(7-azabenzotriazole)-N,N,N′,N′-tetramethyluronium hexafluorophosphate
T₃P: Propylphosphonic cyclic anhydride

### Example 1

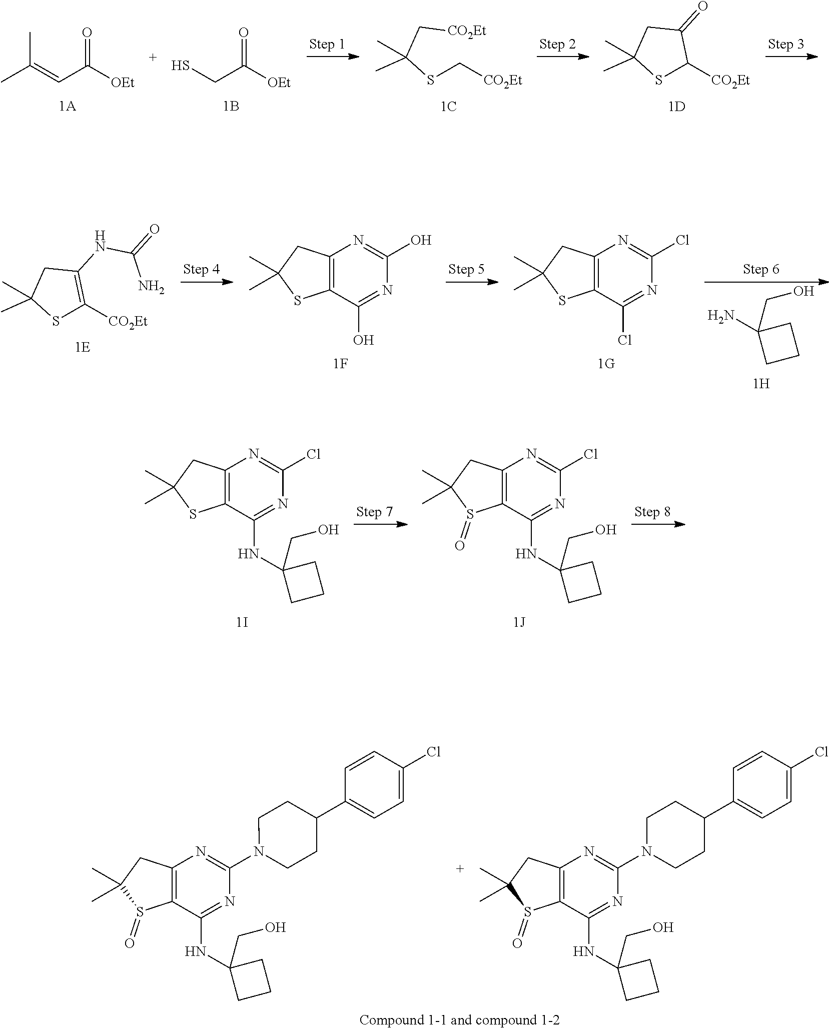

Step 1: In a nitrogen atmosphere, compound 1A (21.6 mL, 155 mmol), compound 1B (15.6 mL, 142 mmol), and 2 mL of piperidine were added to a three-mouth flask, heated to 45° C., and reacted for 48 h. The system was adjusted to acidity by adding 1N hydrochloric acid and extracted three times by adding 200 mL of ethyl acetate. The organic phases were dried over anhydrous sodium sulfate and then concentrated to obtain 36 g of crude compound 1C, which was directly used in the next reaction.

LCMS m/z=249.1 [M+H]⁺.

Step 2: In a nitrogen atmosphere, 80 mL of dichloromethane was added to a three-mouth flask, and the system was cooled to −10° C. Titanium tetrachloride (4.9 mL, 44.4 mmol) was added, followed by isopropanol (3.4 mL, 44.4 mmol) slowly, and the mixture was reacted for 30 min. Crude compound 1C (10 g) was dissolved in 10 mL of DCM and then slowly added to the system, and triethylamine (17 mL, 121 mmol) was then added and reacted at −10° C. for 2 h. After the raw material disappeared, the reaction was quenched by adding 42 mL of 3N hydrochloric acid, and the system was then heated to room temperature and extracted three times by adding 200 mL of ethyl acetate. The organic phase was dried over anhydrous sodium sulfate and then concentrated to obtain 8 g of crude compound 1D, which was directly used in the next reaction.

¹H NMR (400 MHz, CDCl₃) δ 4.36 (s, 1H), 4.25-4.19 (m, 2H), 2.80-2.73 (m, 1H), 2.66-2.58 (m, 1H), 1.58 (s, 3H), 1.49 (s, 3H), 1.32-1.27 (m, 3H).

Step 3: 8 g of crude compound 1D was dissolved in a mixed solvent of methanol:concentrated hydrochloric acid=150 mL:15 mL, and urea (24 g, 400 mmol) was added. After the system was heated to 90° C. and then reacted for 24 h, a solid generated in the system was collected, washed twice with water, and dried to obtain 6.4 g of crude compound 1E, which was directly used in the next reaction.

LCMS m/z=245.1 [M+H]⁺.

Step 4: 6.4 g of crude compound 1E was added to a round-bottom flask, 100 mL of water and sodium hydroxide (8 g, 200 mmol) were added, and the mixture was heated to 85° C. and reacted for 24 h. The system was adjusted to acidity by adding hydrochloric acid, and a solid generated in the system was collected and dried to obtain 3 g of crude compound 1F. The compound was directly used for the next reaction.

LCMS m/z=196.9 [M−H]⁻.

Step 5: In a nitrogen atmosphere, 3 g of crude compound 1F was dissolved in 60 mL of acetonitrile, phosphorus oxychloride (5.7 mL, 61 mmol) was added, and the mixture was heated to 90° C. and reacted for 48 h. After cooling, the system was poured into 300 mL of water, and the generated solid was collected and dried to obtain 1 g of crude compound 1G.

LCMS m/z=235.0 and 237.0 [M+H]⁺.

Step 6: In a nitrogen atmosphere, 1 g of crude compound 1G was dissolved in 30 mL of acetonitrile, 1.5 g of a crude salt of compound 1H (see patent WO 2013/026797 A1 for the method) and triethylamine (5 mL, 36 mmol) were added, and the mixture was heated to 80° C. and then reacted for 18 h. The reaction system was cooled to room temperature and concentrated. Then, the concentrated product was extracted three times by adding 50 mL of ethyl acetate. The organic phases were dried over anhydrous sodium sulfate and then concentrated to obtain 740 mg of crude compound 11, which was directly used in the next reaction.

LCMS m/z=300.1 [M+H]⁺.

Step 7: 740 mg of crude compound 11 was dissolved in 30 mL of acetic acid, 2 mL of 30% hydrogen peroxide was added, and the mixture was reacted at room temperature for 2 h. After the raw material disappeared, the system was extracted three times by adding 50 mL of ethyl acetate. The organic phases were dried over anhydrous sodium sulfate and then concentrated to obtain 540 mg of crude compound 1J, which was directly used in the next reaction.

LCMS m/z=316.1 [M+H]⁺.

Step 8: In a nitrogen atmosphere, 270 mg of crude compound 1J, 4-(4-chlorophenyl)piperidine (220 mg, 1.12 mmol), and DIPEA (0.86 mL, 5.2 mmol) were dissolved in 30 mL of 1,4-dioxane, and the mixture was heated to 100° C. and reacted for 18 h. The reaction system was concentrated and then separated by thin-layer chromatography to obtain 246 mg of crude racemic compound 1, which was subjected to chiral resolution to obtain compound 1-1 (77 mg, 19%) and compound 1-2 (70 mg, 17%).

LCMS m/z=475.2 [M+H]⁺.

¹H NMR (400 MHz, DMSO-d6) δ 7.35-7.25 (m, 5H), 4.85-4.75 (m, 3H), 3.75-3.70 (m, 2H), 3.14-3.07 (m, 1H), 2.96-2.78 (m, 3H), 2.72-2.65 (m, 1H), 2.42-2.29 (m, 2H), 2.19-2.10 (m, 2H), 1.85-1.68 (m, 4H), 1.57-1.44 (m, 2H), 1.36 (s, 3H), 1.18 (s, 3H).

Chiral resolution method: instrument: Waters 150 SFC; column: Chiralpak Column (250×30 mm, I.D 30 mm, 10 μm particle size); mobile phase: A for CO2 and B for MeOH; gradient: 30% phase B isocratic elution; flow rate: 80 mL/min; back pressure: 100 bar; column temperature: 25° C.; wavelength: 220 nm; retention time: compound 1-1: 1.160 min and compound 1-1: 1.701 min.

### Example 2

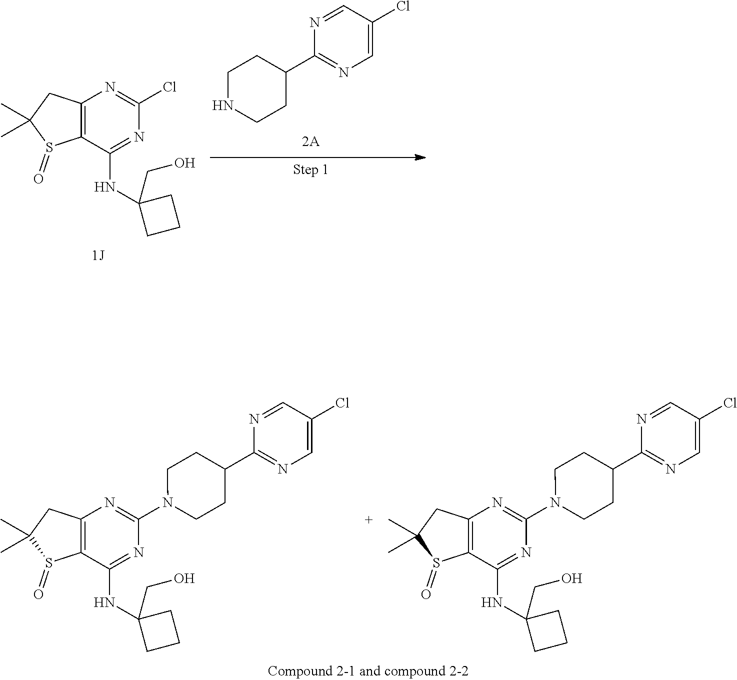 [SMILES: CC1(C)Cc2nc(Cl)nc(NC3(CO)CCC3)c2S1=O ; Clc1cnc(C2CCNCC2)nc1 ; CC1(C)Cc2nc(N3CCC(c4ncc(Cl)cn4)CC3)nc(NC3(CO)CCC3)c2[S@@]1=O ; CC1(C)Cc2nc(N3CCC(c4ncc(Cl)cn4)CC3)nc(NC3(CO)CCC3)c2[S@]1=O]

Step 1: In a nitrogen atmosphere, 270 mg of crude compound 1J, crude compound 2A (320 mg, see US 20140228286 A1), and DIPEA (0.86 mL, 5.2 mmol) were dissolved in 30 mL of 1,4-dioxane, and the mixture was heated to 100° C. and reacted for 18 h. The reaction system was concentrated and then separated by thin-layer chromatography to obtain 226 mg of crude racemic compound 2, which was subjected to chiral resolution to obtain compound 2-1 (54 mg, 13%) and compound 2-2 (40 mg, 10%).

LCMS m/z=477.2 [M+H]⁺.

¹H NMR (400 MHz, DMSO-d6) δ 8.86 (s, 2H), 7.31 (s, 1H), 4.86-4.80 (m, 1H), 4.73-4.64 (m, 2H), 3.76-3.69 (m, 2H), 3.23-3.00 (m, 4H), 2.73-2.65 (m, 1H), 2.41-2.28 (m, 2H), 2.19-2.11 (m, 2H), 2.02-1.92 (m, 2H), 1.84-1.58 (m, 4H), 1.35 (s, 3H), 1.18 (s, 3H).

Chiral resolution method: instrument: Waters 150 SFC; column: Chiralpak Column (250×30 mm, I.D 30 mm, 10 μm particle size); mobile phase: A for CO₂ and B for MeOH; gradient: 30% phase B isocratic elution; flow rate: 70 mL/min; back pressure: 100 bar; column temperature: 25° C.; wavelength: 220 nm; retention time: compound 2-1: 1.063 min and compound 2-2: 1.546 min.

### Example 3

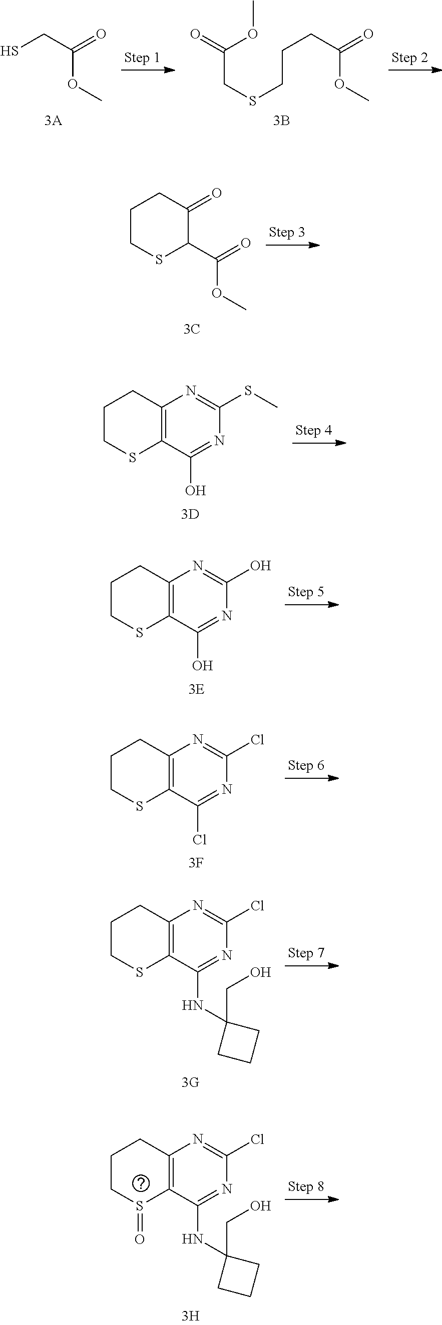

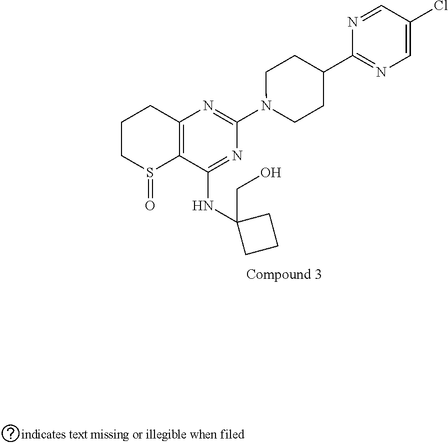 [SMILES: O=S1CCCc2nc(N3CCC(c4ncc(Cl)cn4)CC3)nc(NC3(CO)CCC3)c21]

Using compound 3A and methyl 4-chlorobutyrate as raw materials, compound 3 (35 mg, 22%) was obtained according to the operation of Example 1.

¹H NMR (400 MHZ, DMSO-d6) δ 8.86 (s, 2H), 6.48 (s, 1H), 4.88 (t, 1H), 4.67 (d, 2H), 3.70 (m, 2H), 3.17 (s, 1H), 3.00-3.06 (m, 3H), 2.92 (m, 1H), 2.38 (d, 4H), 2.13 (s, 2H), 1.96 (d, 3H), 1.87-1.71 (m, 2H), 1.58-1.67 (m, 2H).

LCMS (ESI): =463.2 [M+H]⁺.

### Example 4

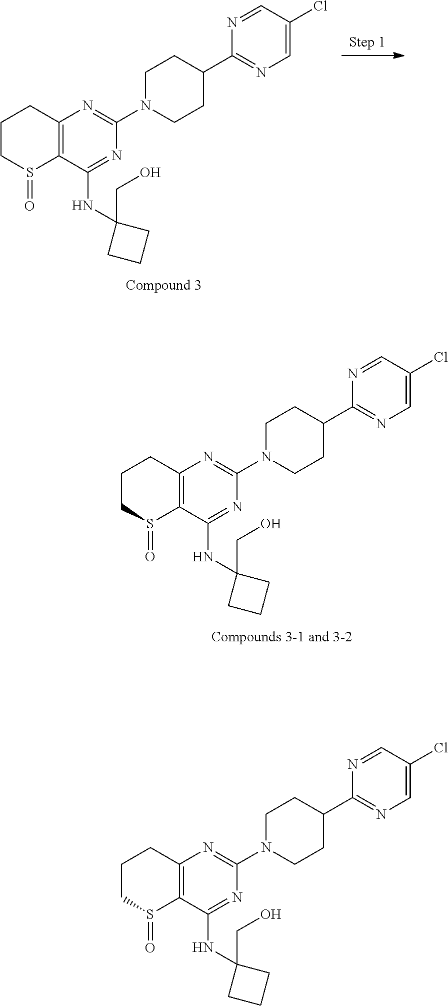 [SMILES: O=S1CCCc2nc(N3CCC(c4ncc(Cl)cn4)CC3)nc(NC3(CO)CCC3)c21 ; O=[S@]1CCCc2nc(N3CCC(c4ncc(Cl)cn4)CC3)nc(NC3(CO)CCC3)c21 ; O=[S@@]1CCCc2nc(N3CCC(c4ncc(Cl)cn4)CC3)nc(NC3(CO)CCC3)c21]

Step 1: Compound 3 was subjected to chiral resolution to prepare compounds 3-1 (26.1 mg) and 3-2 (21.1 mg, 22%).

Chiral resolution method: instrument: Waters 150 SFC; column: Chiralpak Column (250×30 mm, I.D 30 mm, 10 μm particle size); mobile phase: A for CO₂ and B for IPA+CAN (0.1% NH₃·H₂O); gradient: 70% phase B isocratic elution; flow rate: 100 mL/min; back pressure: 100 bar; column temperature: 25° C.; wavelength: 220 nm.

Compound 3-1, retention time: 1.328 min. ¹H NMR (400 MHZ, DMSO-d6) δ 8.86 (s, 2H), 6.48 (s, 1H), 4.88 (t, 1H), 4.67 (d, 2H), 3.70 (m, 2H), 3.17 (s, 1H), 3.00-3.06 (m, 3H), 2.92 (m, 1H), 2.38 (d, 4H), 2.13 (s, 2H), 1.96 (d, 3H), 1.87-1.71 (m, 2H), 1.58-1.67 (m, 2H).

LCMS (ESI): =463.2 [M+H]⁺.

Compound 3-2, retention time: 2.125 min. ¹H NMR (400 MHZ, DMSO-d6) δ 8.86 (s, 2H), 6.48 (s, 1H), 4.88 (t, 1H), 4.67 (d, 2H), 3.70 (m, 2H), 3.17 (s, 1H), 3.00-3.06 (m, 3H), 2.92 (m, 1H), 2.38 (d, 4H), 2.13 (s, 2H), 1.96 (d, 3H), 1.87-1.71 (m, 2H), 1.58-1.67 (m, 2H).

LCMS (ESI): =463.2 [M+H]⁺.

### Example 5

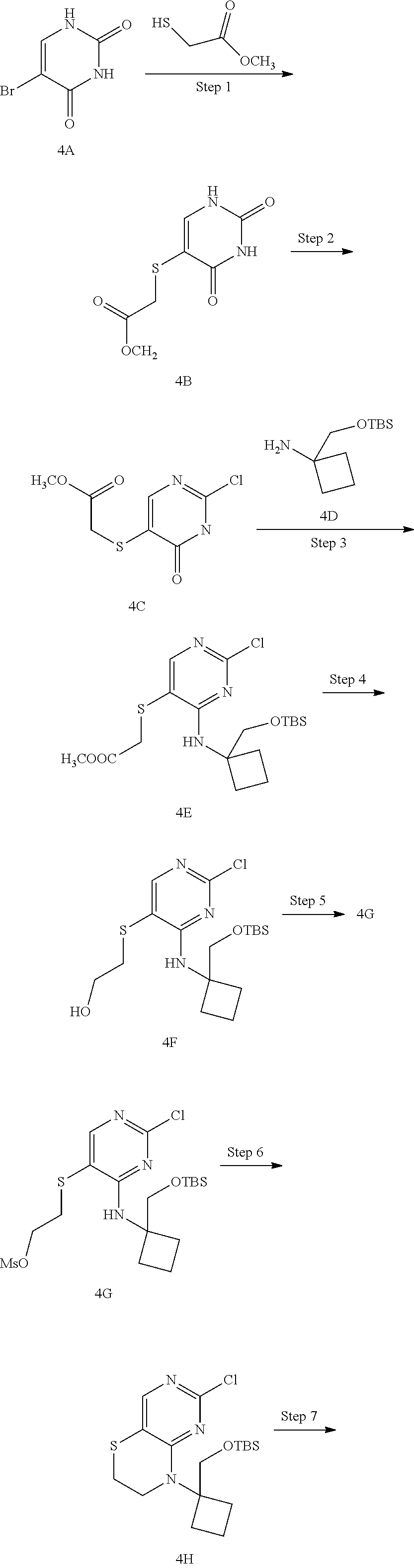

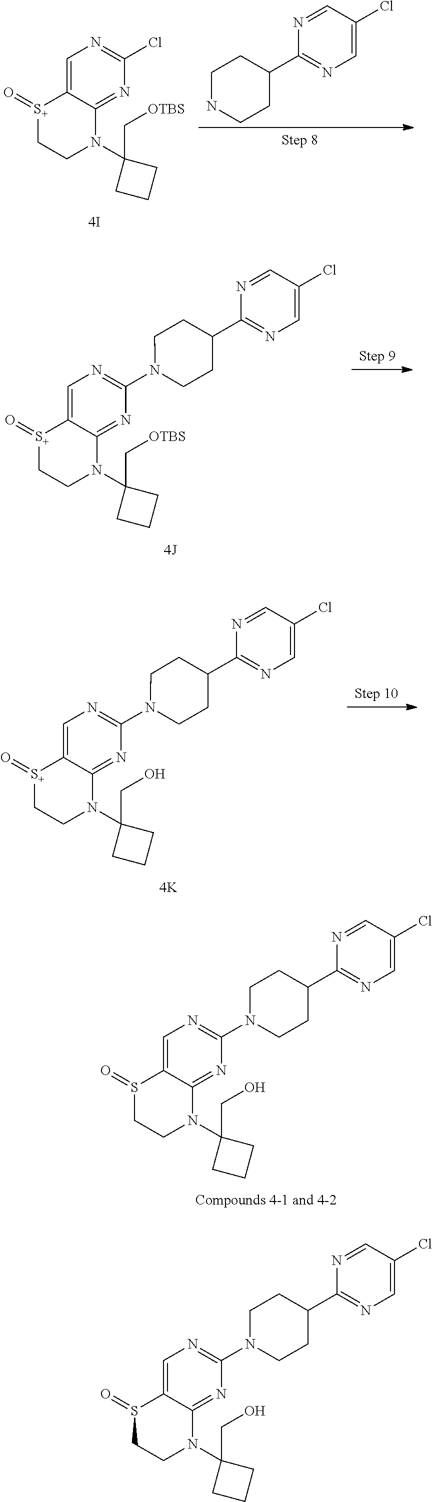

Step 1: The raw material 4A (15 g, 78.50 mmol), methyl thioacetate (8.33 g, 78.50 mmol), potassium carbonate (27.12 g, 196.25 mmol), and tetrabutylammonium sulfate (8 g, 23.55 mmol) were separately added to 400 mL of an N,N-dimethylformamide solvent, stirred at room temperature overnight, and then filtered. The filtrate was concentrated under reduced pressure to obtain the target compound, and the crude product was directly used in the next reaction.

LCMS m/z=217.1 [M+1]⁺

Step 2: The raw material 4B (2.9 g, 13.41 mmol) was added to thionyl chloride (30 mL). The mixture was heated to 90° C., stirred for 4 hours, then concentrated to remove excess thionyl chloride, dissolved with ethyl acetate, and washed with a saturated sodium bicarbonate solution. The filtrate was dried, and after filtration and concentration, the target compound 4C (3.1 g, yield 91%) was separated by silica gel column chromatography (PE:EA (v/v)=1:0-6:1).

LCMS m/z=253.0 [M+1]⁺

¹H NMR (400 MHz, CDCl₃) δ8.58 (s, 1H), 3.72 (s, 3H), 3.59 (s, 2H).

Step 3: The raw material 4C (3.9 g, 15.41 mmol), the raw material 4D (3.32 g, 15.41 mmol), and triethylamine (3.12 g, 30.82 mmol) were added to the solvent acetonitrile (70 mL), then heated to 50° C., stirred for 16 hours, cooled, and concentrated, and the concentrated product was then diluted by adding water, extracted with ethyl acetate, and dried over anhydrous sodium sulfate. After filtration, the filtrate was concentrated and then separated by silica gel column chromatography (PE:EA (v/v)=1:0-4:1) to obtain the target compound 4E (2.6 g, yield 39%).

LCMS m/z=432.1 [M+1]⁺

Step 4: 4E (1.3 g, 3.01 mmol) was added to dry tetrahydrofuran (20 mL), and the reagent sodium borohydride (0.68 g, 18.06 mmol) was added in an ice bath. The mixture was then slowly heated to room temperature and stirred for 16 hours. The reaction was quenched by adding water, and the system was extracted with ethyl acetate, and dried over anhydrous sodium sulfate. After filtration, the filtrate was concentrated and then separated by silica gel column chromatography (PE:EA (v/v)=1:0-2:3) to obtain the target compound 4F (0.7 g, yield 57%).

LCMS m/z=404.2 [M+1]⁺

Step 5: The raw material 4F (0.6 g, 1.49 mmol) was added to dry dichloromethane (10 mL), and the reagents triethylamine (0.45 g, 4.44 mmol) and methanesulfonyl chloride (0.34 g, 2.97 mmol) were added in an ice bath. The mixture was then slowly heated to room temperature and stirred for 1 hour, and the reaction was quenched by adding water. The system was extracted with dichloromethane, washed with a saturated sodium bicarbonate solution and a sodium chloride aqueous solution, respectively, and dried over and anhydrous sodium sulfate. After filtration, the filtrate was concentrated to obtain the target compound 4F (0.85 g, yield 100%).

LCMS m/z=482.1 [M+1]⁺

Step 6: The raw material 4G (0.85 g, 1.76 mmol) was added to the solvent N,N-dimethylformamide (10 mL), and the reagent sodium hydride (0.28 g, 7.04 mmol, 60% in oil) was added in an ice bath. The mixture was then slowly heated to room temperature and stirred for 1 hour. The reaction was quenched by adding water, and the system was extracted with ethyl acetate, and dried over anhydrous sodium sulfate. After filtration, the filtrate was concentrated and then separated by silica gel column chromatography (PE:EA (v/v)=1:0-8:1) to obtain the target compound 4H (0.45 g, yield 66%).

LCMS m/z=386.1 [M+1]⁺

¹H NMR (400 MHz, CDCl₃) δ7.77 (s, 1H), 3.99 (s, 2H), 3.77-3.80 (m, 2H), 2.83-2.85 (m, 2H), 2.23-2.28 (m, 4H), 1.67-1.74 (m, 2H), 0.85 (s, 9H), 0.0 (s, 6H).

Step 7: The raw material 4H (400 mg, 1.04 mmol), 1,1′-binaphthyl-2,2′-bisphenylphosphine (29 mg, 0.1 mmol), water (19 mg, 1.04 mmol), and tetraisopropyl titanate (14 mg, 0.051 mmol) were successively added to the solvent dichloromethane (10 mL) and stirred at room temperature for half an hour. Tert-butyl hydroperoxide (120 mg, 1.35 mmol) was then added, and the mixture was further stirred for 3 hours, then diluted by adding water, and then extracted with dichloromethane. After washing with saturated sodium bicarbonate followed by drying and filtration, the filtrate was concentrated and then purified and separated by silica gel column chromatography (PE:EA (v/v)=1:0-2:3) to obtain the title compound 41 (0.40 g, yield 96%).

LCMS m/z=402.1 [M+1]⁺

Step 8: The raw material 41 (0.4 g, 0.99 mmol), the raw material 2A (0.29 g, 1.48 mmol), and DIPEA (0.38 g, 2.94 mmol) were added to the solvent 1,4-dioxane (20 mL), then heated to 95° C., stirred for 16 hours, cooled, and concentrated, and the concentrated product was then diluted by adding water, extracted with ethyl acetate, and dried over anhydrous sodium sulfate. After filtration, the filtrate was concentrated and then separated by silica gel column chromatography (PE:EA (v/v)=1:0-4:1) to obtain the target compound 4J (0.5 g, yield 90%).

LCMS m/z=563.2 [M+1]⁺

Step 9: The raw material 4J (0.6 g, 1.07 mmol) was dissolved in the solvent tetrahydrofuran (20 mL), and tetrabutylammonium fluoride solution (1.6 mL, 1.60 mmol, 1 M) was then added. The mixture was stirred at room temperature for 2 hours, diluted by adding water, then extracted with ethyl acetate, and dried over anhydrous sodium sulfate. After filtration, the filtrate was concentrated and then purified and separated by silica gel column chromatography (DCM:dihydrous methanol (v/v)=1:0-10:1) to obtain the title compound 4K (0.3 g, yield 62%).

LCMS m/z=449.2 [M+1]⁺

¹H NMR (400 MHz, DMSO-d₆) δ8.85 (s, 1H), 8.16 (s, 1H), 4.93 (t, 1H), 4.62-4.65 (m, 2H), 3.84-3.92 (m, 2H), 3.74-3.79 (m, 1H), 3.56-3.62 (m, 1H), 3.16-3.22 (m, 1H), 2.98-3.08 (m, 3H), 2.62-2.68 (m, 1H), 2.38-2.41 (m, 1H), 2.24-2.30 (m, 3H), 1.95 (d, 2H), 1.59-1.73 (m, 4H).

Step 10: The raw material 4K (300 mg, 0.67 mmol) was purified by chiral HPLC resolution to obtain the target compound 4-1 (100 mg) and the target compound 4-2 (100 mg).

Instrument name: Waters 150 SFC; chromatographic column: Chiralcel OD Column (250×30 mm, I.D 30 mm, 10 μm particle size); mobile phase: A for CO₂ and B for EtOH+ACN (0.1% NH₃·H₂O); gradient: 40% B phase isocratic elution; flow rate: 100 mL/min; column pressure: 100 bar; column temperature: 25° C.; absorption wavelength: 220 nm; cycle time: about 2.9 min; sample preparation: dissolution with acetonitrile.

Peaking time of compound 4-1: 0.578 min

LCMS m/z=449.2 [M+1]⁺

¹H NMR (400 MHz, DMSO-d₆) δ8.85 (s, 1H), 8.16 (s, 1H), 4.93 (t, 1H), 4.62-4.65 (m, 2H), 3.84-3.92 (m, 2H), 3.74-3.79 (m, 1H), 3.56-3.62 (m, 1H), 3.16-3.22 (m, 1H), 2.98-3.08 (m, 3H), 2.62-2.68 (m, 1H), 2.38-2.41 (m, 1H), 2.24-2.30 (m, 3H), 1.95 (d, 2H), 1.59-1.73 (m, 4H).

Peaking time of compound 4-2: 0.812 min

LCMS m/z=449.2 [M+1]⁺

¹H NMR (400 MHz, DMSO-d₆) δ8.85 (s, 1H), 8.16 (s, 1H), 4.93 (t, 1H), 4.62-4.65 (m, 2H), 3.84-3.92 (m, 2H), 3.74-3.79 (m, 1H), 3.56-3.62 (m, 1H), 3.16-3.22 (m, 1H), 2.98-3.08 (m, 3H), 2.62-2.68 (m, 1H), 2.38-2.41 (m, 1H), 2.24-2.30 (m, 3H), 1.95 (d, 2H), 1.59-1.73 (m, 4H).

### Example 6

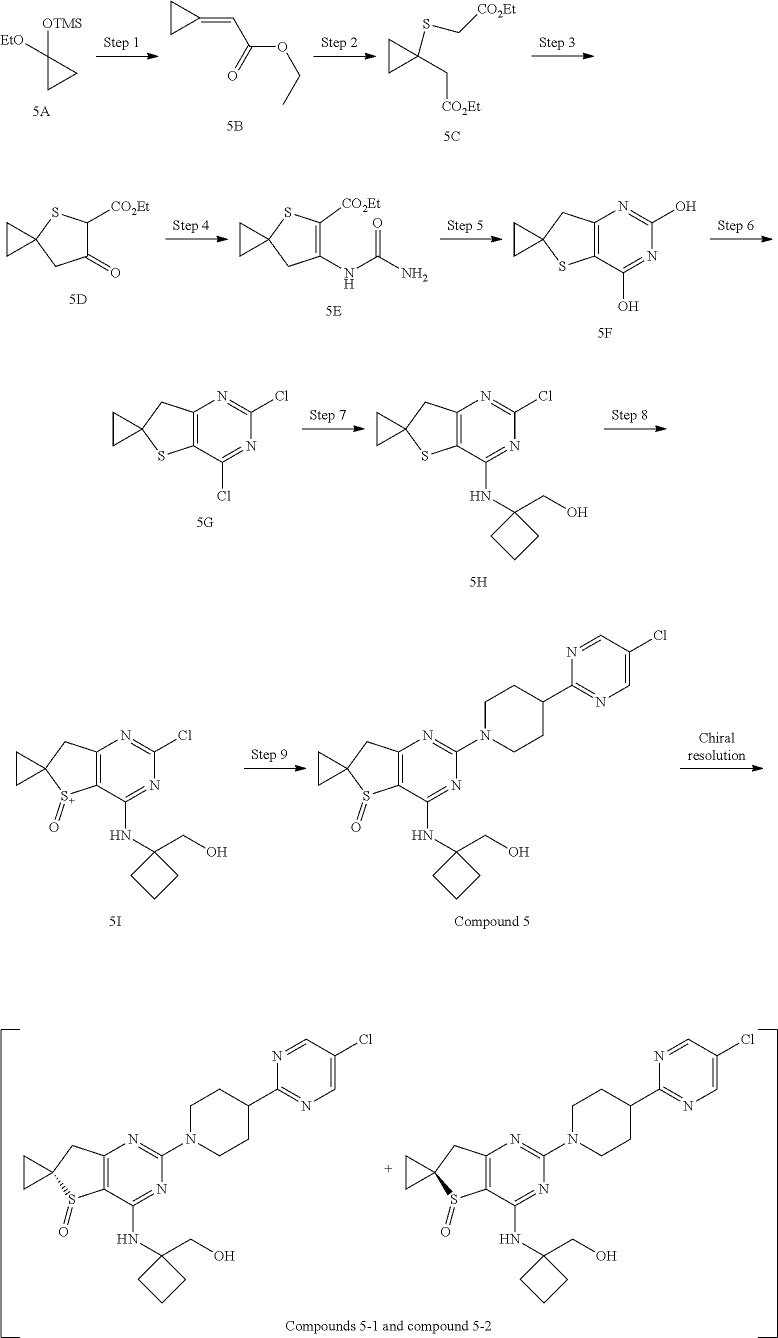

Step 1: Ethoxyformylmethylene triphenylphosphine (14 g, 40.23 mol) dissolved in dichloromethane (6 ml) was added to (1-ethoxycyclopropyloxy)trimethylsilane (7 g, 40.23 mmol) and acetic acid (1.21 g, 20.12 mol) dissolved in tetraethylene glycol dimethyl ether (30 ml) in a 150 ml sealed tube and reacted for 3 h in an oil bath at 100° C. After the reaction was complete, the system was cooled to room temperature. After the reaction was complete, dichloromethane was removed by direct concentration, and the residue was quickly separated and purified by column chromatography (eluent ratio: EA/PE=0-10%) to obtain the target compound 5B (4 g, 79%).

Using compound 5B and ethyl thioacetate as raw materials, compound 5 (50 mg, 33%) was obtained according to the operation of Example 1.

### Chiral Resolution of Compound 5:

Compound 5 (50 mg) was taken for resolution, and after separation, compound 5-1 (retention time: 1.257 s, 15 mg, ee %=100%) and compound 5-2 (retention time: 1.553 s, 17 mg, ee %=100%) were obtained.

### Resolution Conditions:

instrument: Waters 150 MGM; column: DAICEL CHIRALPAK AD;
mobile phases: A for CO₂ and B for EtOH (0.1% NH3·H2O)); gradient: B 40%; flow: 120 mL/min; back pressure: 100 bar;
column temperature: 35° C.; wavelength: 220 nm; period: 13 min;

### Compound 5-1:

¹H NMR (400 MHz, CDCl₃) δ=8.62 (s, 2H), 5.85 (s, 1H), 4.79 (d, 2H), 3.87 (s, 2H), 3.67 (d, 1H), 3.17 (tt, 1H), 3.07 (t, 2H), 2.73 (d, 1H), 2.29 (t, 4H), 2.06 (d, 2H), 1.92-1.80 (m, 3H), 1.61-1.57 (m, 1H), 1.39-1.30 (m, 2H), 1.11 (t, 1H), 1.08-1.04 (m, 1H), 0.84 (d, 1H).

### Compound 5-2:

¹H NMR (400 MHz, CDCl₃) δ=8.62 (s, 2H), 6.00 (s, 1H), 4.80 (d, 2H), 3.86 (d, 2H), 3.68 (d, 1H), 3.17 (tt, 3.8, 1H), 3.07 (t, 2H), 2.72 (d, 1H), 2.35-2.24 (m, 4H), 2.06 (d, 2H), 1.88-1.80 (m, 3H), 1.62-1.56 (m, 1H), 1.39-1.30 (m, 1H), 1.17-1.09 (m, 1H), 1.07-1.00 (m, 1H), 0.92-0.79 (m, 1H).

### Example 7

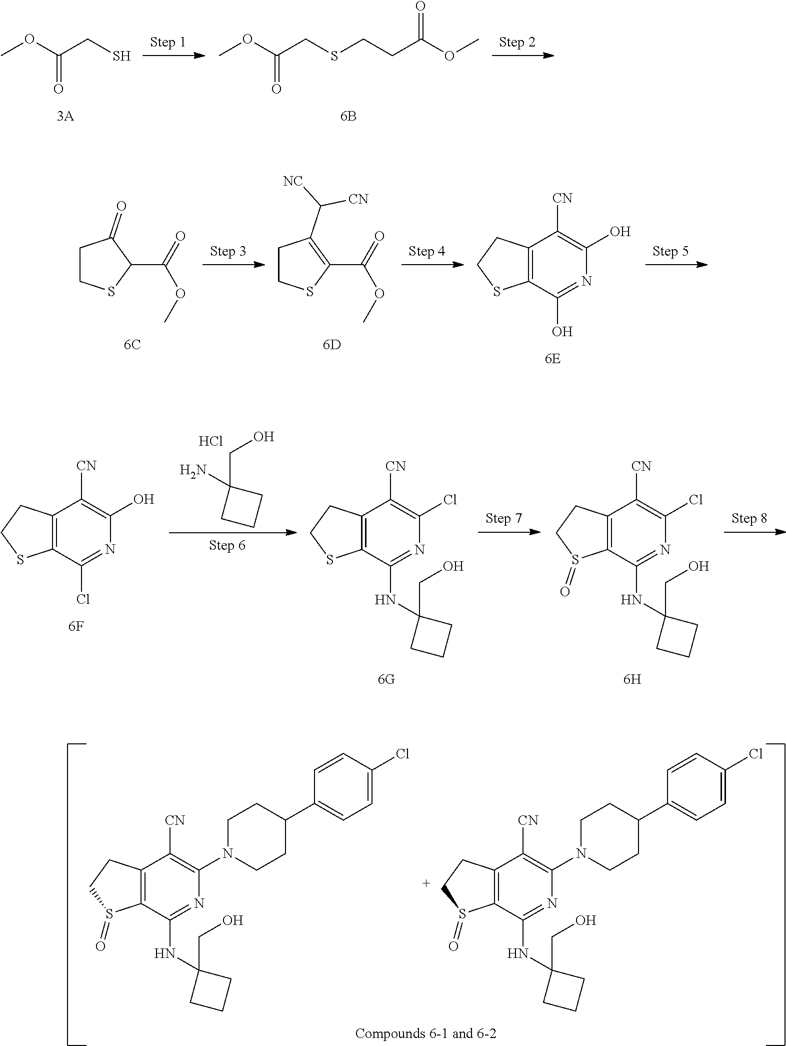

Using compound 3A and methyl acrylate as raw materials, compound 6C (35 g, 97%) was obtained according to the operation of Example 1 (the reactions of the first and second steps).

LC-MS (ESI): m/z=161.1 [M+H]⁺

Step 3: At room temperature, compound 6C (30 g, 187.5 mmol) was dissolved in methanol (300 mL), and malononitrile (18.56 g, 281.2 mmol) and imidazole (38.25 g, 562.5 mmol) were added at room temperature and stirred for 3 h. After the reaction was complete, most of the methanol was spun off, and water (50 mL) was added. After extraction twice with dichloromethane (100 mL×2), the organic phases were combined, washed with a saturated sodium chloride aqueous solution, and dried over anhydrous sodium sulfate. After filtration, the filtrate was concentrated, and the residue was then separated and purified by silica gel column chromatography (eluent ratio: EA:PE=0-50%) to obtain the title compound 6D (26.7 g, 69%).

LC-MS (ESI): m/z=209.1 [M+H]⁺

Step 4: In a nitrogen atmosphere, compound 6D (12 g, 61.86 mmol) was dissolved in a hydrogen chloride-dioxane solution (4M, 100 mL) at room temperature, the mixture was then heated to 100° C. and reacted overnight, whereupon a large amount of a solid precipitated. After the reaction was complete, filtration was carried out, and the obtained solid was spin-dried to obtain crude compound 6E (9 g, 80%). The obtained compound was directly used in the next reaction without further purification.

LC-MS (ESI): m/z=195.1 [M+H]⁺

Step 5: At room temperature, compound 6E (2 g, 10.31 mmol) was added to a 120 mL sealed tube charged with phosphorus oxychloride (20 mL), benzyltrimethylammonium chloride (3.83 g, 20.62 mmol) was added at room temperature, and the mixture was then heated to 150° C. and reacted for 7 h. After the reaction was complete, the reaction liquid was slowly added to a saturated sodium bicarbonate solution (100 mL) in an ice bath and extracted twice with ethyl acetate (150 mL×2). The organic phases were combined, washed with a saturated sodium chloride aqueous solution, and dried over anhydrous sodium sulfate. After filtration, the filtrate was concentrated, and the residue was then separated and purified by silica gel column chromatography (eluent ratio: EA:PE=0-50%) to obtain the title compound 6F (1.1 g, 46%).

Step 6: At room temperature, compound 6F (1.1 g, 4.78 mmol) was dissolved in a 1,4-dioxane solution (12 mL), N,N-diisopropylethylamine (1.23 g, 9.56 mmol) and (1-aminocyclobutyl)methanol hydrochloride (0.65 g, 4.78 mmol) were added at room temperature, and the mixture was then heated to 100° C. and reacted overnight. After the reaction was complete, the reaction liquid was concentrated, and the residue was then separated and purified by silica gel column chromatography (eluent ratio: MeOH:DCM=0-10%) to obtain the title compound 6G (600 mg, 43%).

LC-MS (ESI): m/z=296.3 [M+H]⁺

Step 7: At room temperature, compound 6G (600 mg, 2.03 mmol) was dissolved in acetic acid (30 mL), and 30% hydrogen peroxide (1.0 mL) was added room temperature and reacted for 2 h. After the reaction was complete, a saturated sodium bicarbonate solution (30 mL) was added, and the system was extracted twice with dichloromethane (50 ml×2). The organic phases were combined, washed with a saturated sodium chloride aqueous solution, and dried over anhydrous sodium sulfate. After filtration, the filtrate was concentrated, and the residue was then separated and purified by silica gel column chromatography (eluent ratio: MeOH:DCM=0-10%) to obtain the title compound 6H (300 mg, 47%).

LC-MS (ESI): m/z=312.3 [M+H]⁺

Step 8: At room temperature, compound 6H (150 mg, 0.48 mmol) was dissolved in a 1,4-dioxane solution (2.0 mL), N,N-diisopropylethylamine (124 mg, 0.96 mmol) and 4-(4-chlorophenyl)piperidine (94 mg, 0.48 mmol) were added at room temperature, and the mixture was then heated to 100° C. and reacted overnight. After the reaction was complete, the crude product resulting from concentration under reduced pressure was separated by preparative HPLC to obtain racemate 6 (70 mg, 31%). Separation method: 1. instrument: waters 2767 preparative liquid chromatographic instrument; chromatographic column: SunFire® Prep C18 (19 mm×250 mm); 2. The sample was filtered with a 0.45 μm filter to prepare a sample liquid. 3. Preparative chromatography conditions: a. composition of mobile phases A and B: mobile phase A: acetonitrile; mobile phase B: water (containing 0.1% ammonium acetate); b. gradient elution, mobile phase A: with a content of 10-55%; c. flow: 12 mL/min.

Chiral preparative resolution of racemate, preparative chromatographic conditions: 1. instrument: Waters 150 SFC; 2. chromatographic column: Chiralpak IC-Column (250×30 mm, I.D 30 mm, 10 μm particle size); 3. mobile phase system: A for CO₂ and B for MeOH (0.1% NH₃·H₂O); 4. gradient: B 35%; and 5. flow rate: 100 mL/min. After preparation, the title compound 6-1 (30 mg, 13.3%, retention time: 1.092 min, absolute configuration undetermined) and the title compound 6-2 (31 mg, 13.7%, retention time: 1.761 min, absolute configuration undetermined) were obtained.

Compound 6-1 ¹H NMR (400 MHz, Methanol-d4) δ 7.22-7.16 (m, 2H), 7.16-7.10 (m, 2H), 4.66-4.57 (m, 2H), 3.82 (q, 2H), 3.71-3.61 (m, 1H), 3.40-3.26 (m, 2H), 3.17-3.02 (m, 3H), 2.79 (tt, 1H), 2.35-2.08 (m, 4H), 1.90-1.70 (m, 4H), 1.70-1.57 (m, 2H).

LC-MS (ESI): m/z=471.1 [M+H]⁺

Compound 6-2 ¹H NMR (400 MHz, Methanol-d4) δ 7.33-7.27 (m, 2H), 7.26-7.21 (m, 2H), 4.77-4.67 (m, 2H), 3.92 (q, 2H), 3.81-3.70 (m, 1H), 3.49-3.36 (m, 2H), 3.27-3.12 (m, 3H), 2.89 (tt, 1H), 2.43-2.24 (m, 4H), 1.98-1.82 (m, 4H), 1.80-1.68 (m, 2H).

LC-MS (ESI): m/z=471.1 [M+H]⁺

### Example 8

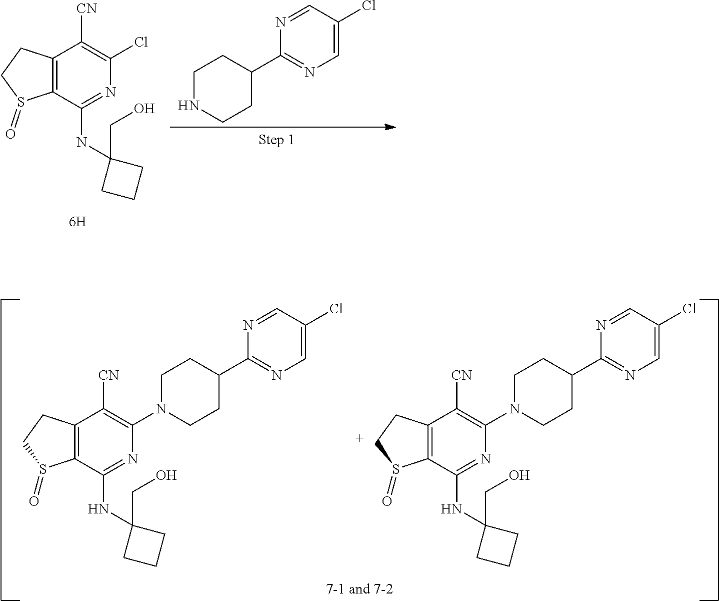

Using compounds 6H and 2A as raw materials, racemate 7 (80 mg, 35%) was obtained according to the operation of Example 2. Separation method: 1. instrument: waters 2767 preparative liquid chromatographic instrument; chromatographic column: SunFire® Prep C18 (19 mm×250 mm); 2. The sample was filtered with a 0.45 μm filter to prepare a sample liquid. 3. Preparative chromatography conditions: a. composition of mobile phases A and B: mobile phase A: acetonitrile; mobile phase B: water (containing 0.1% ammonium acetate); b. gradient elution, mobile phase A: with a content of 10-55%; c. flow: 12 mL/min.

Chiral preparative resolution of racemate, preparative chromatographic conditions: 1. instrument: Waters 150 SFC; 2. chromatographic column: Chiralpak IC-Column (250×30 mm, I.D 30 mm, 10 μm particle size); 3. mobile phase system: A for CO₂ and B for MeOH (0.1% NH₃·H₂O); 4. gradient: B 35%; and 5. flow rate: 100 mL/min. After preparation, the title compound 7-1 (31 mg, 13.6%, retention time: 1.400 min, absolute configuration undetermined) and the title compound 7-2 (33 mg, 14.5%, retention time: 1.681 min, absolute configuration undetermined) were obtained.

Compound 7-1 ¹H NMR (400 MHz, Methanol-d4) δ 8.75 (s, 2H), 4.66 (dt, 2H), 3.92 (q, 2H), 3.81-3.71 (m, 1H), 3.50-3.36 (m, 2H), 3.30-3.19 (m, 4H), 2.42-2.24 (m, 4H), 2.10 (dd, 2H), 1.99-1.83 (m, 4H).

LC-MS (ESI): m/z=473.2 [M+H]⁺

Compound 7-2 ¹H NMR (400 MHz, Methanol-d4) δ 8.75 (s, 2H), 4.67 (dd, 2H), 4.55 (s, 1H), 3.92 (q, 2H), 3.82-3.70 (m, 1H), 3.50-3.36 (m, 2H), 3.27-3.21 (m, 3H), 2.42-2.24 (m, 4H), 2.10 (dd, 2H), 2.01-1.79 (m, 4H).

LC-MS (ESI): m/z=473.2 [M+H]⁺

### Example 9

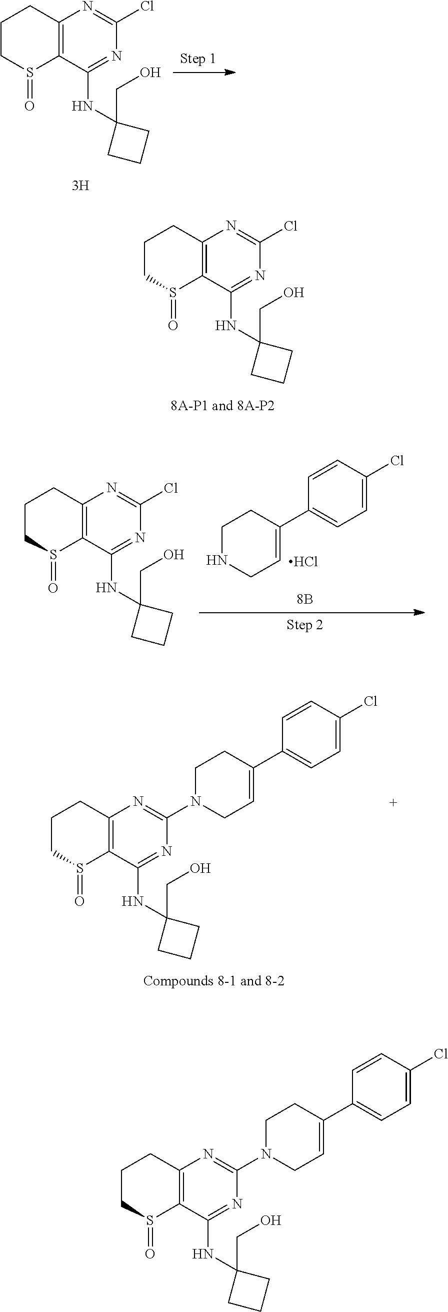

Step 1: Compound 3H (3.7 g, 12.3 mmol) was separated by a chiral column to obtain compounds 8A-P1 (1.1 g, 30%) and 8A-P2 (0.9 g, 24%). Chiral resolution method: instrument: Waters 150 SFC; column: Chiralpak IC Column (250×30 mm, I.D 30 mm, 10 μm particle size); mobile phase: A for CO2 and B for IPA+ACN (0.1% NH₃·H₂O); gradient: 45% phase B isocratic elution; flow rate: 100 mL/min; back pressure: 100 bar; column temperature: 25° C.; wavelength: 220 nm.

Compound 8A-P1, retention time: 0.614 min ¹H NMR (400 MHZ, DMSO-d6) δ 7.58 (s, 1H), 4.91 (t, 1H), 3.68 (d, 2H), 3.25-3.18 (m, 1H), 3.11-3.03 (m, 1H), 2.86-2.76 (m, 1H), 2.73-2.61 (m, 1H), 2.37-2.23 (m, 3H), 2.17 (m, 2H), 2.09-1.93 (m, 1H), 1.87-1.68 (m, 2H).

LCMS (ESI): =302.1 [M+H]⁺.

Compound 8A-P2, retention time: 1.148 min. ¹H NMR (400 MHZ, DMSO-d6) δ 7.58 (s, 1H), 4.91 (t, 1H), 3.68 (d, 2H), 3.25-3.18 (m, 1H), 3.11-3.03 (m, 1H), 2.86-2.76 (m, 1H), 2.73-2.61 (m, 1H), 2.37-2.23 (m, 3H), 2.17 (m, 2H), 2.09-1.93 (m, 1H), 1.87-1.68 (m, 2H).

LCMS (ESI): =302.1 [M+H]⁺.

Step 2: Compound 8A-P1 (0.10 g, 0.33 mmol), 8B (77 mg, 0.40 mmol, see CN 103145607 for the synthesis method), and DIPEA (0.13 mg, 0.99 mmol) were dissolved in dioxane (10 mL) and stirred at 85° C. for 16 h. After the reaction was complete, the system was concentrated under reduced pressure and subjected to HPLC preparation to obtain compound 8-1 (60 mg, 40%).

Preparation method: instrument: Abilene 1290 infinity II preparative liquid chromatogram; chromatographic column: XSelect® Prep C18 (19 mm×250 mm); the sample was dissolved in DMF and filtered with a 0.45 μm filter to prepare a sample solution. Preparative chromatography conditions: composition of mobile phases A and B: mobile phase A: 5 mmol ammonium acetate, mobile phase B: methanol; gradient elution, mobile phase B with content from 50% to 85%; flow: 15 ml/min; elution time: 18 min; retention time: 16.8.

Compound 8-1, ¹H NMR (400 MHZ, DMSO-d6) δ 7.48 (d, 2H), 7.40 (d, 2H), 6.53 (s, 1H), 6.31 (s, 1H), 4.91 (t, 1H), 4.33 (s, 2H), 3.94 (t, 2H), 3.73 (d, 2H), 3.10-3.05 (m, 1H), 2.95-2.88 (m, 1H), 2.74-2.51 (m, 4H), 2.46-2.33 (m, 3H), 2.17 (brs, 2H), 2.02-1.91 (m, 1H), 1.87-1.77 (m, 2H).

The synthesis, preparation, and separation of compound 8-2 (45 mg, 36%) were the same as those of compound 8-1.

Compound 8-2, ¹H NMR (400 MHZ, DMSO-d6) δ 7.48 (d, 2H), 7.40 (d, 2H), 6.53 (s, 1H), 6.31 (s, 1H), 4.91 (t, 1H), 4.33 (s, 2H), 3.94 (t, 2H), 3.73 (d, 2H), 3.10-3.05 (m, 1H), 2.95-2.88 (m, 1H), 2.74-2.51 (m, 4H), 2.46-2.33 (m, 3H), 2.17 (brs, 2H), 2.02-1.91 (m, 1H), 1.87-1.77 (m, 2H).

LCMS (ESI): =459.50 [M+H]⁺.

### Example 10

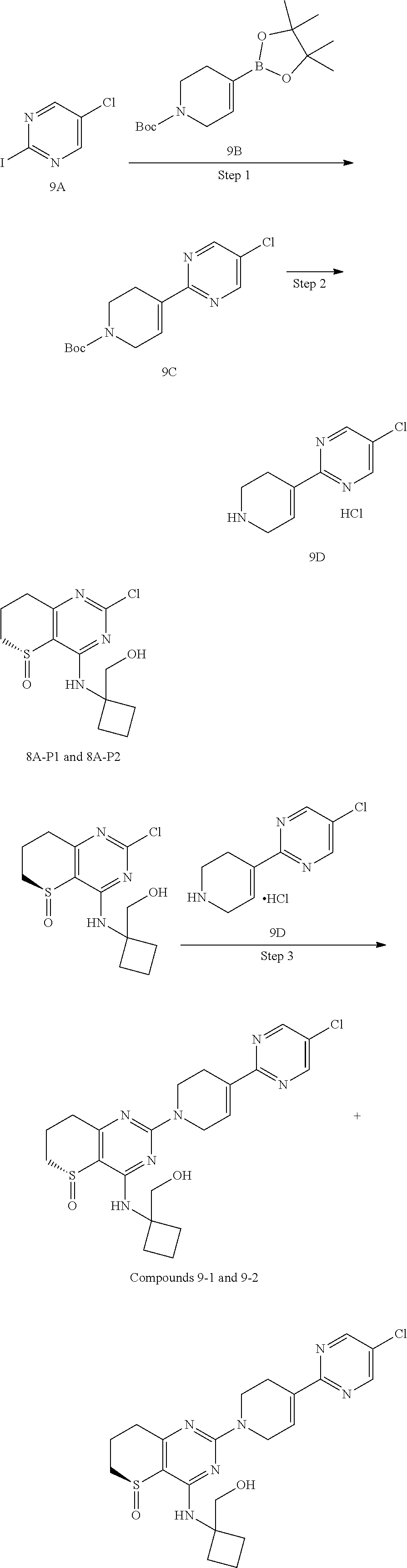

Step 1: Substrate 9A (5.0 g, 20.77 mmol), dioxane (100 mL), and water (20 mL) were added to a 250 mL single-mouth flask and dissolved, sodium carbonate (6.6 g, 62.31 mmol), 9B (7.1 g, 22.85 mmol), and [1,1′-bis(diphenylphosphine)ferrocene]palladium dichloride (1.5 g, 2.08 mmol) were added, and the mixture was stirred at 100 degrees Celsius overnight under nitrogen protection. The reaction liquid was diluted by adding water (50 mL) and suction-filtered with diatomite. The filtrate was extracted with ethyl acetate (2×100 mL), and the organic phases were combined, dried over anhydrous sodium sulfate, concentrated, and subjected to column chromatography (petroleum ether:ethyl acetate (v/v)=10:1) to obtain the title compound 9C (5.0 g, 81%).

LC-MS (ESI): m/z=240.1 [M−56+H]+.

Step 2: Substrate 9C (1.0 g, 3.38 mmol), methanol (5 mL), and hydrogen chloride dioxane (4 M, 5 mL) were added to a 50 mL single-mouth flask, dissolved, and stirred at room temperature for 3 h, and the reaction liquid was concentrated to obtain the title compound 9D (0.79 g, 100%).

LC-MS (ESI): m/z=196.2 [M+H]⁺.

Step 3: Compound 8A-P1 (0.10 g, 0.33 mmol), 9D (77 mg, 0.40 mmol), and DIPEA (0.13 mg, 0.99 mmol) were dissolved in dioxane (10 mL) and stirred at 85° C. for 16 h. After the reaction was complete, the solvent was spun off, and HPLC preparation was carried out obtain compound 9-1 (70 mg, 46%).

Preparation method: instrument: Abilene 1290 infinity II preparative liquid chromatogram; chromatographic column: XSelect® Prep C18 (19 mm×250 mm); the sample was dissolved in DMF and filtered with a 0.45 μm filter to prepare a sample solution. Preparative chromatography conditions: composition of mobile phases A and B: mobile phase A: 5 mmol ammonium acetate, mobile phase B: methanol; gradient elution, mobile phase B with content from 50% to 85%; flow: 15 ml/min; elution time: 18 min; retention time: 16.5 min.

The synthesis, preparation, and separation conditions of compound 9-2 (60 mg, 48%) were the same as those of compound 9-1.

Compound 9-1, ¹H NMR (400 MHZ, DMSO-d6) δ 8.89 (s, 2H), 7.32-7.24 (m, 1H), 6.54 (s, 1H), 4.90 (t, 1H), 4.43 (s, 2H), 3.94 (t, 2H), 3.73 (d, 2H), 3.10-3.04 (m, 1H), 2.95-2.88 (m, 1H), 2.71-2.53 (m, 4H), 2.45-2.27 (m, 3H), 2.22-2.09 (m, 2H), 2.02-1.91 (m, 1H), 1.88-1.69 (m, 2H).

Compound 9-2, ¹H NMR (400 MHZ, DMSO-d6) δ 8.89 (s, 2H), 7.32-7.24 (m, 1H), 6.54 (s, 1H), 4.90 (t, 1H), 4.43 (s, 2H), 3.94 (t, 2H), 3.73 (d, 2H), 3.10-3.04 (m, 1H), 2.95-2.88 (m, 1H), 2.71-2.53 (m, 4H), 2.45-2.27 (m, 3H), 2.22-2.09 (m, 2H), 2.02-1.91 (m, 1H), 1.88-1.69 (m, 2H).

LCMS (ESI): =461.50 [M+H]⁺.

### Example 11

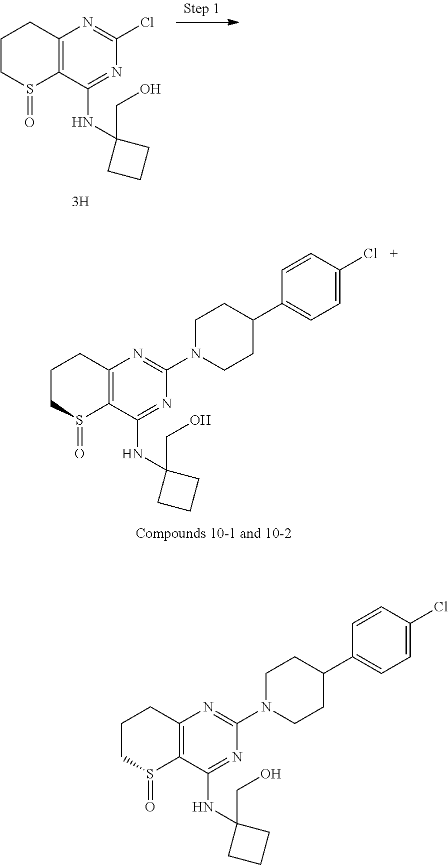 [SMILES: O=S1CCCc2nc(Cl)nc(NC3(CO)CCC3)c21 ; O=[S@]1CCCc2nc(N3CCC(c4ccc(Cl)cc4)CC3)nc(NC3(CO)CCC3)c21 ; O=[S@@]1CCCc2nc(N3CCC(c4ccc(Cl)cc4)CC3)nc(NC3(CO)CCC3)c21]

Step 1: Compound 3H (150 mg, 0.50 mmol), 4-(4-chlorophenyl)piperidine hydrochloride (131 mg, 0.55 mmol), and DIPEA (260 mg, 2.00 mmol) were dissolved in dioxane and reacted at 90° C. for 16 h. After the reaction was complete, the solvent was spun off, and SFC preparation was carried out to obtain compounds 10-1 (44.3 mg, 19%) and 10-2 (26.5 mg, 11%).

Chiral resolution method: instrument: Waters 150 SFC; column: Chiralcel OX Column (250×30 mm, I.D 30 mm, 10 μm particle size); mobile phase: A for CO₂ and B for EtOH+ACN; gradient: 45% phase B isocratic elution; flow rate: 120 mL/min; back pressure: 100 bar; column temperature: 25° C.; wavelength: 220 nm.

Compound 10-1, retention time: 1.128 min. ¹H NMR (400 MHZ, DMSO-d6) δ 7.38-7.29 (m, 2H), 7.30-7.20 (m, 2H), 6.47 (s, 1H), 4.90 (t, 1H), 4.77 (d, 2H), 3.70 (d, 2H), 3.10-3.03 (m, 1H), 2.54-2.51 (m, 1H), 2.98-2.79 (m, 4H), 2.72-2.61 (m, 1H), 2.45-2.30 (m, 3H), 2.18-2.08 (m, 2H), 2.03-1.89 (m, 1H), 1.83-1.71 (m, 4H), 1.55-1.41 (m, 2H).

LCMS (ESI): =461.2 [M+H]⁺.

Compound 10-2, retention time: 1.609 min. ¹H NMR (400 MHZ, DMSO-d6) δ 7.38-7.29 (m, 2H), 7.30-7.20 (m, 2H), 6.47 (s, 1H), 4.90 (t, 1H), 4.77 (d, 2H), 3.70 (d, 2H), 3.10-3.03 (m, 1H), 2.54-2.51 (m, 1H), 2.98-2.79 (m, 4H), 2.72-2.61 (m, 1H), 2.45-2.30 (m, 3H), 2.18-2.08 (m, 2H), 2.03-1.89 (m, 1H), 1.83-1.71 (m, 4H), 1.55-1.41 (m, 2H).

LCMS (ESI): =461.2 [M+H]⁺.

### Example 12

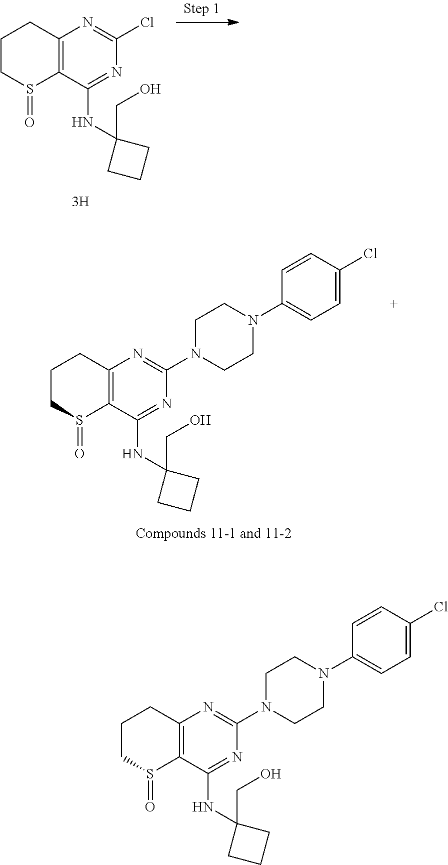 [SMILES: O=S1CCCc2nc(Cl)nc(NC3(CO)CCC3)c21 ; O=[S@]1CCCc2nc(N3CCN(c4ccc(Cl)cc4)CC3)nc(NC3(CO)CCC3)c21 ; O=[S@@]1CCCc2nc(N3CCN(c4ccc(Cl)cc4)CC3)nc(NC3(CO)CCC3)c21]

Step 1: Compound 3H (150 mg, 0.50 mmol), 4-(4-chlorophenyl)piperazine hydrochloride (131 mg, 0.55 mmol), and DIPEA (260 mg, 2.00 mmol) were dissolved in dioxane and reacted at 90° C. for 16 h. After the reaction was complete, the system was concentrated under reduced pressure and subjected to SFC preparation to obtain compounds 11-1 (29.2 mg, 13%) and 11-2 (39.9 mg, 17%).

Chiral resolution method: instrument: Waters 150 SFC; column: Chiralcel OX Column (250×30 mm, I.D 30 mm, 10 μm particle size); mobile phase: A for CO₂ and B for EtOH+ACN (0.1% NH₃·H₂O); gradient: 45% phase B isocratic elution; flow rate: 120 mL/min; back pressure: 100 bar; column temperature: 25° C.; wavelength: 220 nm.

Compound 11-1, retention time: 1.192 min. ¹H NMR (400 MHZ, DMSO-d6) δ 7.42-7.17 (m, 2H), 7.12-6.84 (m, 2H), 6.55 (s, 1H), 4.89 (t, 1H), 3.84 (t, 4H), 3.71 (d, 2H), 3.18-3.13 (m, 4H), 3.10-3.04 (m, 1H), 2.95-2.87 (m, 1H), 2.74-2.63 (m, 1H), 2.61-2.51 (m, 1H), 2.46-2.23 (m, 3H), 2.23-2.10 (m, 2H), 2.00-1.94 (m, 1H), 1.91-1.67 (m, 2H).

LCMS (ESI): =462.2 [M+H]⁺.

Compound 11-2, retention time: 1.766 min. ¹H NMR (400 MHZ, DMSO-d6) δ 7.42-7.17 (m, 2H), 7.12-6.84 (m, 2H), 6.55 (s, 1H), 4.89 (t, 1H), 3.84 (t, 4H), 3.71 (d, 2H), 3.18-3.13 (m, 4H), 3.10-3.04 (m, 1H), 2.95-2.87 (m, 1H), 2.74-2.63 (m, 1H), 2.61-2.51 (m, 1H), 2.46-2.23 (m, 3H), 2.23-2.10 (m, 2H), 2.00-1.94 (m, 1H), 1.91-1.67 (m, 2H).

LCMS (ESI): =462.2 [M+H]⁺.

### Example 13

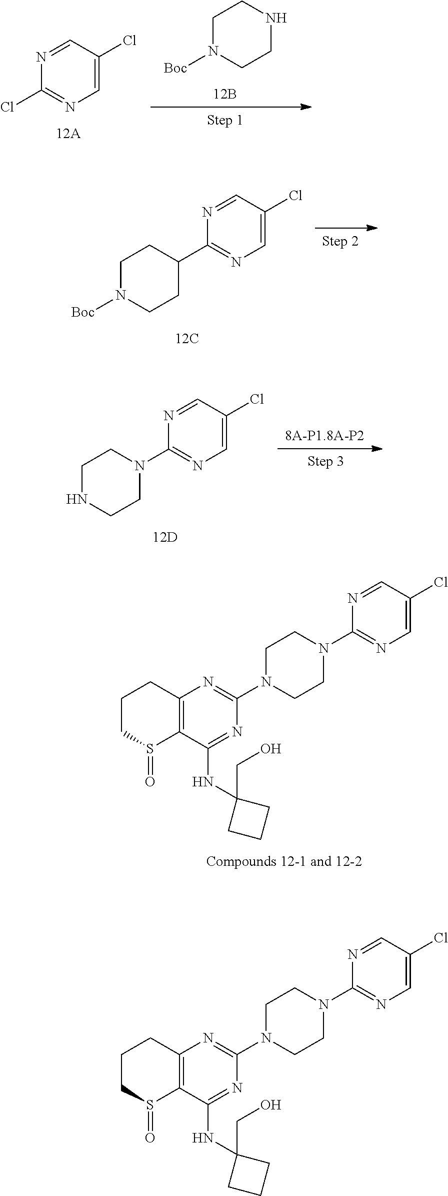 [SMILES: Clc1cnc(Cl)nc1 ; CC(C)(C)OC(=O)N1CCNCC1 ; CC(C)(C)OC(=O)N1CCC(c2ncc(Cl)cn2)CC1 ; Clc1cnc(N2CCNCC2)nc1 ; O=[S@@]1CCCc2nc(N3CCN(c4ncc(Cl)cn4)CC3)nc(NC3(CO)CCC3)c21 ; O=[S@]1CCCc2nc(N3CCN(c4ncc(Cl)cn4)CC3)nc(NC3(CO)CCC3)c21]

Step 1: Compound 12A (3.00 g, 20.10 mmol) and compound 12B (3.74 g, 20.10 mmol) were weighed into a 100 mL single-mouth flask and then dissolved by adding 1,4-dioxane (30 mL), and N,N-diisopropylethylamine (7.79 g, 60.30 mmol) was added to the reaction liquid. After the addition was complete, the mixture was stirred at 80 degrees Celsius for 16 h. After the reaction was complete as determined by a TLC spotting plate (petroleum ether:ethyl acetate=5:1), water (20 mL) was added to the reaction liquid and stirred for 5 min. The liquid was extracted by adding ethyl acetate (20 mL), and the organic phase was separated. The organic phase was dried over anhydrous sodium sulfate, and the crude product was concentrated under reduced pressure and purified by column chromatography (eluents: petroleum ether:ethyl acetate=5:1) to obtain the target compound 12C (3.0 g, yield: 50%).

LCMS m/z=299.1 [M+1]⁺

Step 2: Compound 12C (1.0 g, 3.35 mmol) was weighed into a 100 mL single-mouth flask and then dissolved by adding methanol (10 mL), and a hydrogen chloride dioxane solution (4N, 10 mL) was added. After the addition was complete, the mixture was stirred for 2 h. The organic phase was concentrated under reduced pressure until no liquid dripped out to obtain the target compound 12D (0.80 g, crude).

LCMS m/z=199.1 [M+1]⁺

Step 3: Compound 8A-P1 (80 mg, 0.27 mmol), compound 12D (65 mg, 0.33 mmol), and N,N-diisopropylethylamine (0.10 g, 0.77 mmol) were added to a 100 mL single-mouth flask. After the addition was complete, the system was placed under nitrogen protection and stirred at 90° C. for 16 h, and the reaction liquid was concentrated under reduced pressure to obtain the crude target compound, which was subjected to preparative HPLC to obtain the title compound 12-1 (60 mg, 48%).

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A with a content of 35-80%; c. flow: 15 mL/min; d. elution time: 20 min;
retention time: 13.50 min.

The synthesis, preparation, and separation of compound 12-2 (58 mg, 47%) were the same as those of compound 12-1.

LCMS m/z=464.2 [M+1]⁺

Compound 12-1, ¹H NMR (400 MHZ, DMSO-d6) δ 8.44 (d, 2H), 6.55 (s, 1H), 4.88 (t, 1H), 3.73-3.82 (m, 8H), 3.70 (d, 2H), 3.04-3.10 (m, 1H), 2.88-2.95 (m, 1H), 2.54-2.70 (m, 2H), 2.29-2.44 (m, 3H), 2.10-2.19 (m, 2H), 1.90-2.00 (m, 1H), 1.72-1.85 (m, 2H).

Compound 12-2, ¹H NMR (400 MHZ, DMSO-d6) δ 8.44 (d, 2H), 6.55 (s, 1H), 4.88 (t, 1H), 3.73-3.82 (m, 8H), 3.70 (d, 2H), 3.04-3.10 (m, 1H), 2.88-2.95 (m, 1H), 2.54-2.70 (m, 2H), 2.29-2.44 (m, 3H), 2.10-2.19 (m, 2H), 1.90-2.00 (m, 1H), 1.73-1.87 (m, 2H).

### Example 14

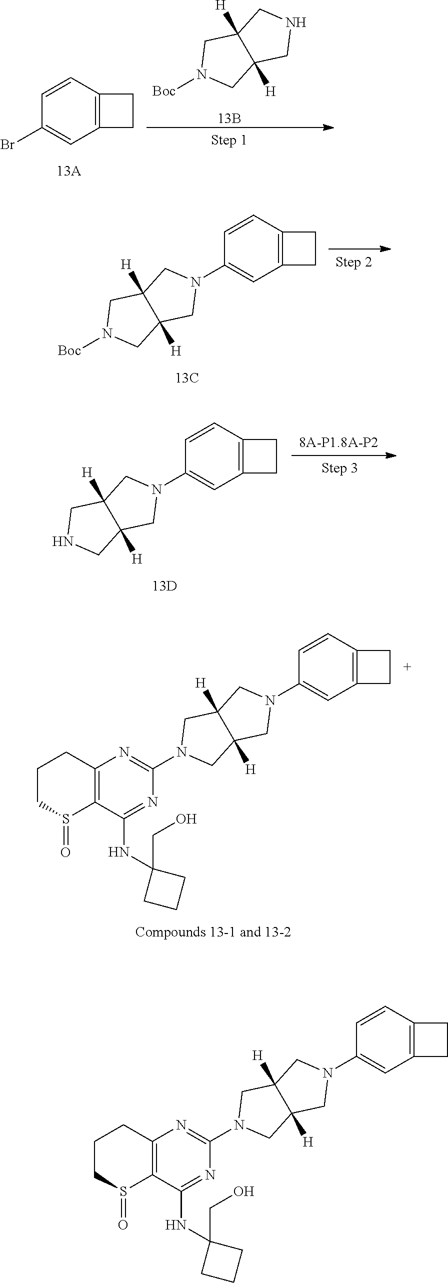

Step 1: Compound 13A (1.00 g, 5.45 mmol) and compound 13B (1.16 g, 5.45 mmol) were weighed into a 100 mL single-mouth flask and then dissolved by adding 1,4-dioxane (30 mL), and sodium tert-butoxide (1.57 g, 16.34 mmol), tris(dibenzylideneacetone)dipalladium (0.15 g, 0.27 mmol), and 1,1′-binaphthyl-2,2′-bisdiphenylphosphine (0.34 g, 0.55 mmol) were added to the reaction liquid. After the addition was complete, the mixture was stirred at 90 degrees Celsius for 16 h. After the reaction was complete as determined by a TLC spotting plate (petroleum ether:ethyl acetate=5:1), water (20 mL) was added to the reaction liquid and stirred for 5 min. The liquid was extracted by adding ethyl acetate (20 mL), and the organic phase was separated. The organic phase was dried over anhydrous sodium sulfate, and the crude product was concentrated under reduced pressure and purified by column chromatography (eluents: petroleum ether:ethyl acetate=5:1) to obtain the target compound 13C (1.0 g, yield: 58%).

LCMS m/z=315.1 [M+1]⁺

Step 2: Compound 13C (0.30 g, 0.95 mmol) was weighed into a 100 mL single-mouth flask and then dissolved by adding methanol (10 mL), and a hydrogen chloride dioxane solution (4N, 10 mL) was added. After the addition was complete, the mixture was stirred for 2 h. The organic phase was concentrated under reduced pressure until no liquid dripped out to obtain the target compound 13D (0.20 g, crude).

LCMS m/z=215.1 [M+1]⁺

Step 3: Compound 8A-P1 (80 mg, 0.27 mmol), compound 13D (69 mg, 0.32 mmol), and N,N-diisopropylethylamine (0.10 g, 0.77 mmol) were added to a 100 mL single-mouth flask. After the addition was complete, the system was placed under nitrogen protection and stirred at 90° C. for 16 h, and the reaction liquid was concentrated under reduced pressure to obtain the crude target compound, which was subjected to preparative HPLC to obtain the title compound 13-1 (12 mg, 9%).

Preparation method: instrument: waters 2767 preparative liquid chromatographic instrument; chromatographic column: SunFire® Prep C18 (19 mm×250 mm); the sample was dissolved in methanol and filtered with a 0.45 μm filter to prepare a sample solution. Preparative chromatography conditions: a. composition of mobile phases A and B: mobile phase A: acetonitrile, and mobile phase B: water (containing 5 mM aqueous ammonia); b. gradient elution, mobile phase A with a content of 45-85%; c. flow: 15 mL/min; d. elution time: 20 min;
retention time: 12.80 min.

The synthesis, preparation, and separation of compound 13-2 (40 mg, 30%) were the same as those of compound 13-1.

LCMS m/z=480.2 [M+1]⁺

Compound 13-1, ¹H NMR (400 MHz, DMSO-d₆) δ 6.84 (d, 1H), 6.45 (s, 1H), 6.33-6.37 (m, 2H), 4.88 (t, 1H), 3.67-3.79 (m, 4H), 3.36-3.46 (m, 4H), 3.11 (d, 2H), 2.97-3.01 (m, 7H), 2.85-2.92 (m, 1H), 2.53-2.67 (m, 2H), 2.25-2.46 (m, 3H), 2.05-2.15 (m, 2H), 1.89-1.98 (m, 1H), 1.69-1.85 (m, 2H).

Compound 13-2, ¹H NMR (400 MHz, DMSO-d₆) δ 6.84 (d, 1H), 6.45 (s, 1H), 6.33-6.37 (m, 2H), 4.88 (t, 1H), 3.67-3.79 (m, 4H), 3.36-3.46 (m, 4H), 3.11 (d, 2H), 2.97-3.01 (m, 7H), 2.85-2.91 (m, 1H), 2.53-2.67 (m, 2H), 2.25-2.46 (m, 3H), 2.05-2.15 (m, 2H), 1.89-1.98 (m, 1H), 1.69-1.84 (m, 2H).

### Example 15

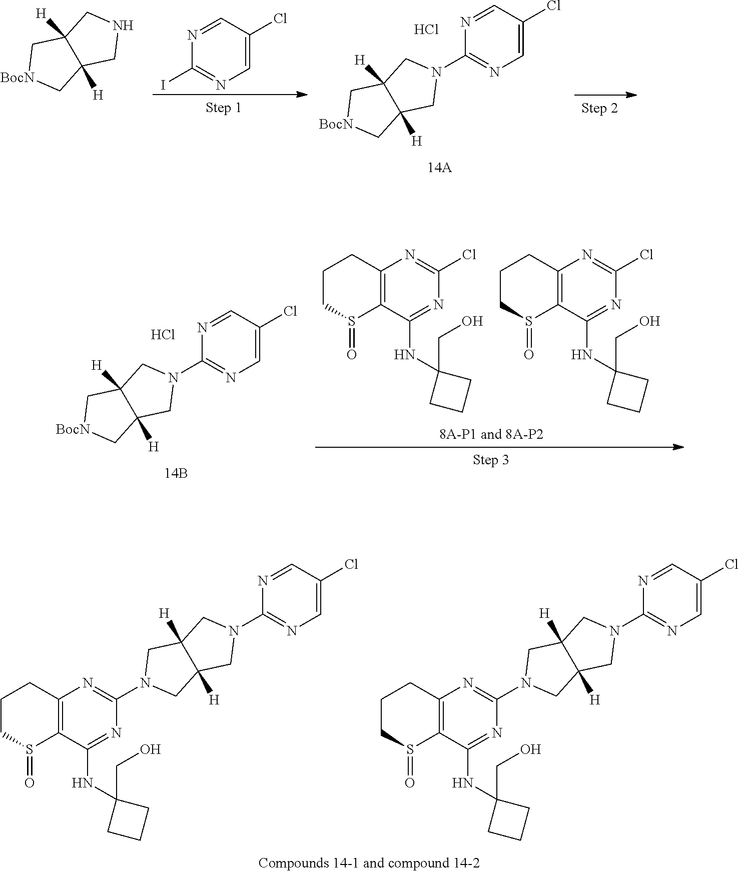

Using the compound tert-butyl (3aR,6aS)-hexahydropyrrolo[3,4-c]pyrrole-2(1H)-carboxylate and 9A as raw materials, compound 14-1 (76 mg, 59.6%) was obtained according to the operation of Example 13.

The synthesis and purification of compound 14-2 (83 mg, 65.1%) were the same as those of compound 14-1.

Compound 14-1: ¹H NMR (400 MHz, DMSO-d₆) δ 8.39 (s, 2H), 6.46 (s, 1H), 4.95-4.81 (m, 1H), 3.81-3.64 (m, 6H), 3.46-3.34 (m, 4H), 3.12-3.00 (m, 3H), 2.95-2.82 (m, 1H), 2.71-2.54 (m, 2H), 2.45-2.25 (m, 3H), 2.18-2.05 (m, 2H), 2.00-1.88 (m, 1H), 1.88-1.64 (m, 2H).

Compound 14-2: ¹H NMR (400 MHz, DMSO-d₆) δ 8.39 (s, 2H), 6.46 (s, 1H), 4.91-4.83 (m, 1H), 3.82-3.62 (m, 6H), 3.47-3.36 (m, 4H), 3.15-3.02 (m, 3H), 2.94-2.81 (m, 1H), 2.70-2.52 (m, 2H), 2.47-2.24 (m, 3H), 2.18-2.05 (m, 2H), 2.03-1.89 (m, 1H), 1.88-1.63 (m, 2H).

LC-MS (ESI): m/z=490.6 [M+H]⁺.

### Example 16

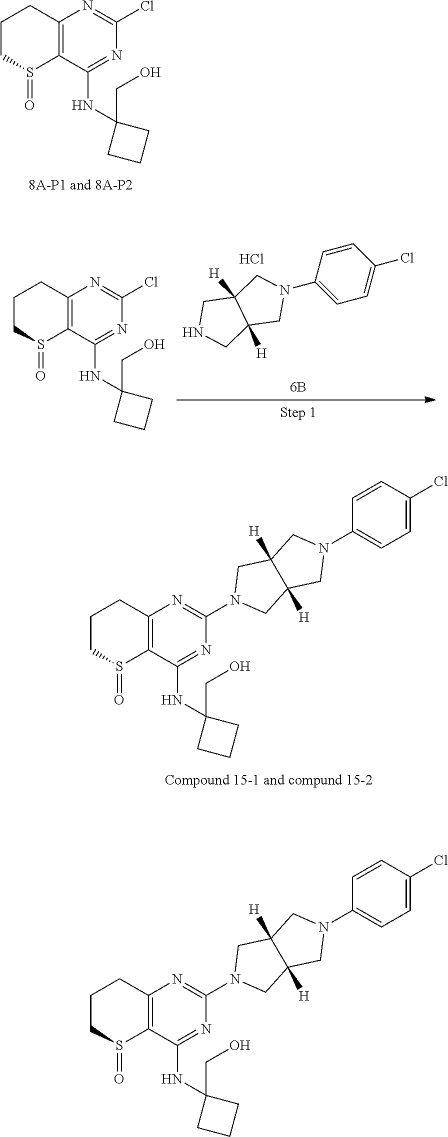 [SMILES: O=[S@@]1CCCc2nc(Cl)nc(NC3(CO)CCC3)c21 ; O=[S@]1CCCc2nc(Cl)nc(NC3(CO)CCC3)c21 ; Clc1ccc(N2C[C@H]3CNC[C@H]3C2)cc1 ; Cl ; O=[S@@]1CCCc2nc(N3C[C@H]4CN(c5ccc(Cl)cc5)C[C@H]4C3)nc(NC3(CO)CCC3)c21 ; O=[S@]1CCCc2nc(N3C[C@H]4CN(c5ccc(Cl)cc5)C[C@H]4C3)nc(NC3(CO)CCC3)c21]

Step 1: 8A-P1 (80 mg, 0.26 mmol) and 6B (120 mg, see WO 2020099886 for the synthesis method) were dissolved in 1,4-dioxane (10 mL), DIPEA (0.5 mL) was added, and the mixture was left at 90° C. overnight. After the raw material was completely reacted as detected by LCMS, the system was concentrated, water (100 mL) was added, and the mixture was extracted with a mixed solution of DCM:MeOH=20:1 (10 mL×3). The organic phases were combined, dried over anhydrous sodium sulfate, concentrated, and then passed through a column (DCM:MeOH=1:0-0:1) to obtain compound 15-1 (84 mg, 66.2%).

The synthesis and purification of compound 15-2 (88 mg, 69.8%) were the same as those of compound 15-1.

Compound 15-1: ¹H NMR (400 MHz, DMSO-d₆) δ 7.21-7.10 (m, 2H), 6.58-6.50 (m, 2H), 6.46 (s, 1H), 4.92-4.85 (m, 1H), 3.84-3.73 (m, 2H), 3.73-3.65 (m, 2H), 3.51-3.43 (m, 2H), 3.43-3.35 (m, 2H), 3.20-3.12 (m, 2H), 3.11-3.00 (m, 3H), 2.93-2.81 (m, 1H), 2.69-2.59 (m, 1H), 2.58-2.51 (m, 1H), 2.47-2.25 (m, 3H), 2.16-2.04 (m, 2H), 1.99-1.88 (m, 1H), 1.87-1.64 (m, 2H).

Compound 15-2: ¹H NMR (400 MHz, DMSO-d₆) δ 7.20-7.11 (m, 2H), 6.58-6.50 (m, 2H), 6.46 (s, 1H), 4.93-4.84 (m, 1H), 3.81-3.72 (m, 2H), 3.72-3.64 (m, 2H), 3.52-3.43 (m, 2H), 3.43-3.34 (m, 2H), 3.22-3.11 (m, 2H), 3.11-3.02 (m, 3H), 2.91-2.80 (m, 1H), 2.68-2.59 (m, 1H), 2.58-2.52 (m, 1H), 2.48-2.29 (m, 3H), 2.16-2.03 (m, 2H), 1.99-1.87 (m, 1H), 1.87-1.64 (m, 2H).

LC-MS (ESI): m/z=488.6 [M+H]⁺.

### Example 17

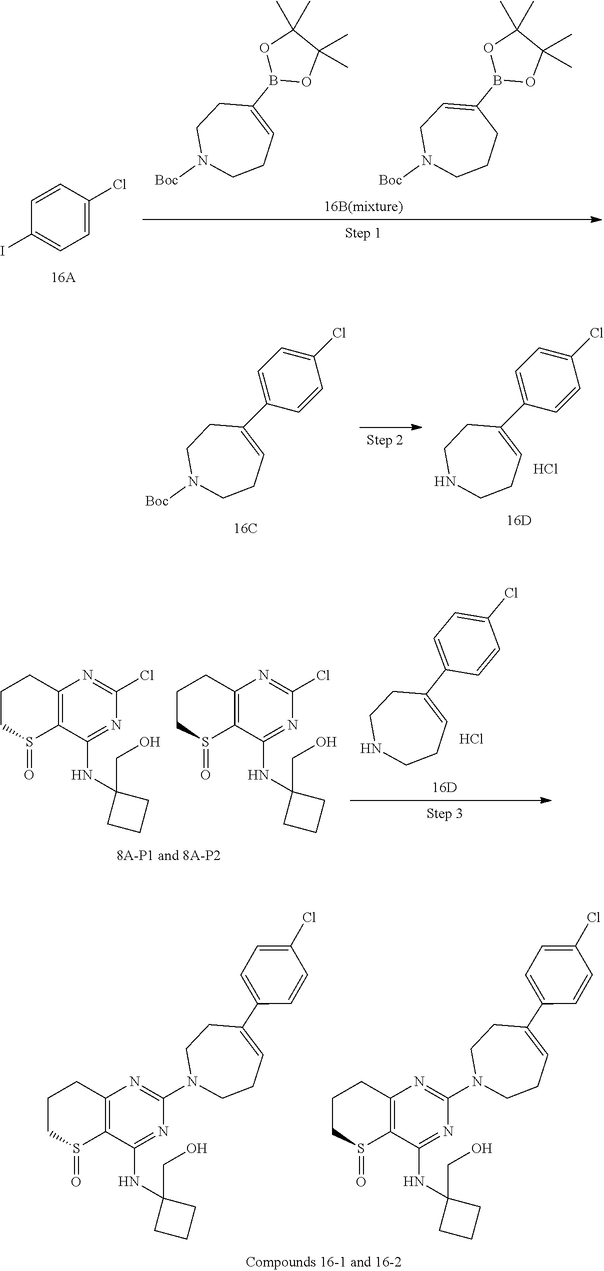

Using compounds 16A and 16B as raw materials, compound 16-1 (50 mg, 20%) was obtained according to the operation of Example 10.

The synthesis (with 8A-P2 as a raw material), preparation, and separation of compound 16-2 (50 mg, 20%) were the same as those of compound 16-1.

Preparation method: instrument: SHIMADZU LC-20AP; chromatographic column: Phenomenex C18; the sample was dissolved with acetonitrile and water and filtered with a 0.45 μm filter to prepare a sample liquid. Preparative chromatography conditions: composition of mobile phases A and B: mobile phase A: 10 mmol ammonium bicarbonate, mobile phase B: acetonitrile; gradient elution, mobile phase B with content from 30% to 60%; flow: 25 ml/min; elution time: 15 min; retention time: 3.3 min.

Compound 16-1, ¹H NMR (400 MHz, DMSO-d₆) δ 7.34 (s, 4H), 6.45 (s, 1H), 6.03 (t, 1H), 4.88 (t, 1H), 3.92 (brs, 2H), 3.83 (brs, 2H), 3.70 (d, 2H), 3.09-3.03 (m, 1H), 2.94-2.87 (m, 1H), 2.72-2.63 (m, 3H), 2.59-2.52 (m, 1H), 2.51-2.45 (m, 2H), 2.43-2.26 (m, 3H), 2.14-2.10 (m, 2H), 1.97-1.92 (m, 1H), 1.83-1.74 (m, 2H).

Compound 16-2, ¹H NMR (400 MHz, DMSO-d₆) δ 7.34 (s, 4H), 6.45 (s, 1H), 6.03 (t, 1H), 4.88 (t, 1H), 3.92 (brs, 2H), 3.83 (brs, 2H), 3.70 (d, 2H), 3.09-3.03 (m, 1H), 2.94-2.87 (m, 1H), 2.72-2.63 (m, 3H), 2.59-2.52 (m, 1H), 2.51-2.45 (m, 2H), 2.43-2.26 (m, 3H), 2.14-2.10 (m, 2H), 1.97-1.92 (m, 1H), 1.83-1.74 (m, 2H).

LCMS (ESI): =473.2 [M+H]⁺.

### Example 18

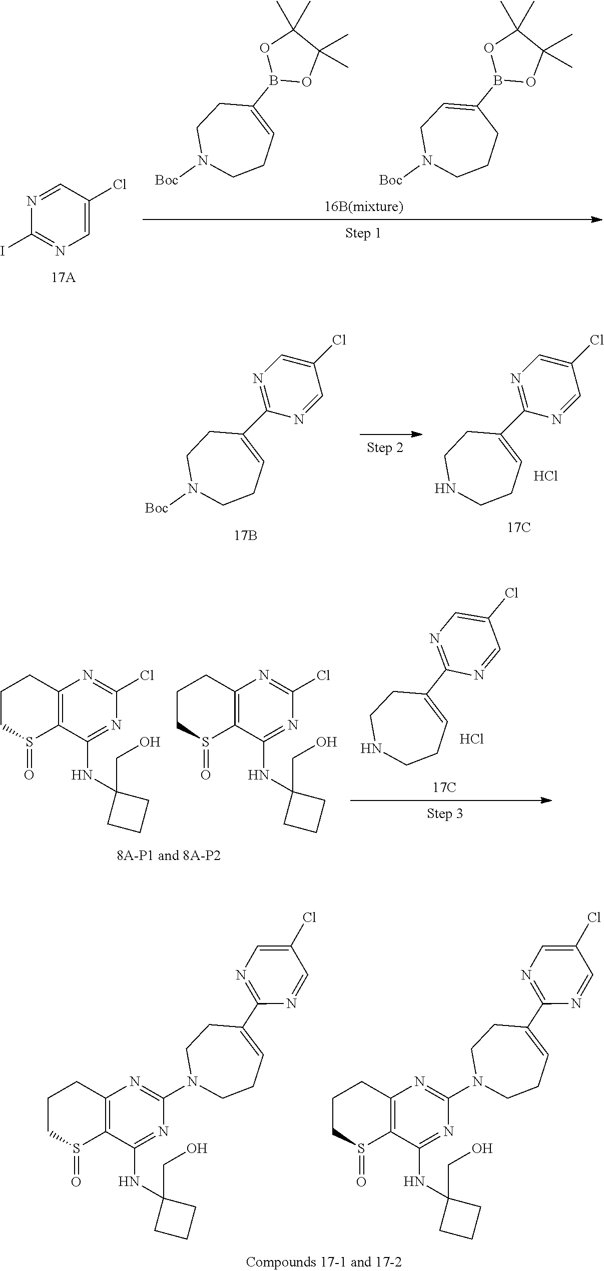

Using 17A and 16B as raw materials, compound 17-1 (30 mg, 12%) was obtained according to the operation of Example 10.

The synthesis (with 8A-P2 as a raw material), preparation, and separation of compound 17-2 (48 mg, 19%) were the same as those of compound 17-1.

Preparation method: instrument: Waters 150 SFC; chromatographic column: Chiralpak IG Column (250×30 mm, I.D 30 mm, 10 μm particle size); preparative chromatography conditions: a. composition of mobile phases A and B: mobile phase A: CO₂, mobile phase B: EtOH+ACN (0.1% NH₃·H₂O); b. isocratic elution, mobile phase B with a content of 65%; c. flow: 100 mL/min; d. elution time: 15 min; e. back pressure: 100 bar; f. column temperature: 25 degrees Celsius.

Compound 17-1, (H NMR (400 MHz, DMSO-d₆) 8.84 (s, 2H), 7.38 (s, 1H), 6.45 (s, 1H), 4.91 (t, 1H), 3.88-3.86 (m, 4H), 3.71 (d, 2H), 3.09-3.00 (m, 3H), 2.91-2.87 (m, 1H), 2.71-2.52 (m, 4H), 2.46-2.24 (m, 3H), 2.16-2.07 (m, 2H), 2.02-1.91 (m, 1H), 1.86-1.68 (m, 2H).

Compound 17-2, ¹H NMR (400 MHz, DMSO-d₆) δ 8.84 (s, 2H), 7.38 (s, 1H), 6.45 (s, 1H), 4.91 (t, 1H), 3.88-3.86 (m, 4H), 3.71 (d, 2H), 3.09-3.00 (m, 3H), 2.91-2.87 (m, 1H), 2.71-2.52 (m, 4H), 2.46-2.24 (m, 3H), 2.16-2.07 (m, 2H), 2.02-1.91 (m, 1H), 1.86-1.68 (m, 2H).

LCMS (ESI): =475.1 [M+H]⁺.

### Example 19

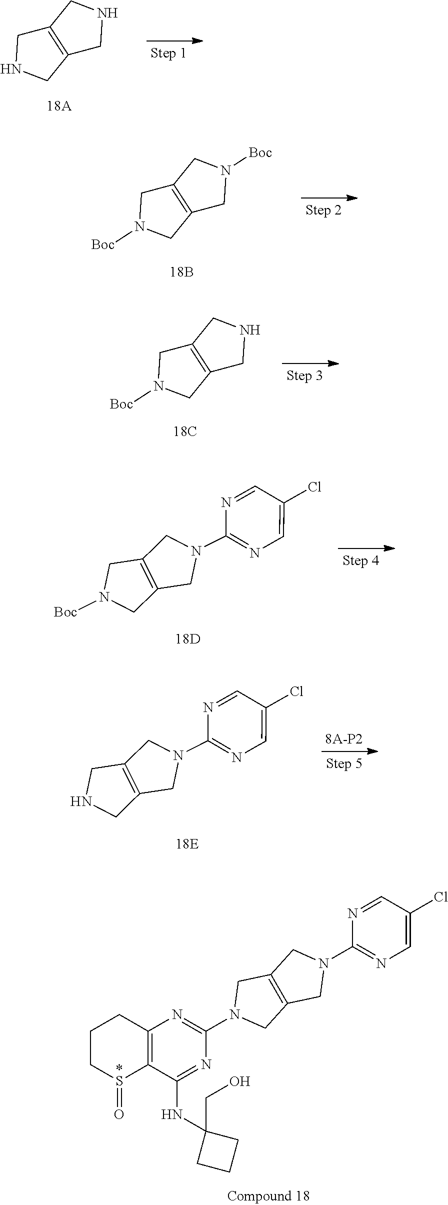

Step 1: Compound 18A (1.50 g, 13.62 mmol) and di-tert-butyl dicarbonate (5.95 g, 27.24 mmol) were weighed into a 100 mL single-mouth flask and then dissolved by adding water (30 mL), and sodium bicarbonate (3.43 g, 40.86 mmol) was added to the reaction liquid. After the addition was complete, the mixture was stirred for 16 h. After the reaction was complete as determined by a TLC spotting plate (petroleum ether:ethyl acetate=5:1), a solid precipitated from the reaction liquid and is directly filtered to obtain the target compound 18B (1.5 g, yield: 35%).

¹H NMR (400 MHZ, DMSO-d6) δ 3.96-4.06 (m, 8H), 1.42 (s, 18H).

Step 2: Compound 18B (1.50 g, 4.83 mmol) and p-toluenesulfonic acid (0.83 g, 4.83 mmol) were weighed into a 100 mL single-mouth flask, then dissolved by adding isopropyl acetate (30 mL), and stirred at 60° C. for 4 h. After the reaction was complete as determined by a TLC spotting plate (petroleum ether:ethyl acetate=3:1), a solid precipitated from the reaction liquid and is directly filtered to obtain the target compound 18C (1.5 g, yield: 100%).

¹H NMR (400 MHZ, DMSO-d₆) δ 3.96-4.03 (m, 8H), 1.42 (s, 9H).

Step 3: Compound 18C (0.30 g, 1.43 mmol) and 2-chloro-5-chloropyrimidine (0.21 g, 1.43 mmol) were weighed into a 100 mL single-mouth flask and then dissolved by adding 1,4-dioxane (20 mL), and N,N-diisopropylethylamine (0.55 g, 4.26 mmol) was added to the reaction liquid. After the addition was complete, the mixture was stirred at 80 degrees Celsius for 16 h. After the reaction was complete as determined by a TLC spotting plate (petroleum ether:ethyl acetate=5:1), water (20 mL) was added to the reaction liquid and stirred for 5 min. The liquid was extracted by adding ethyl acetate (20 mL), and the organic phase was separated. The organic phase was dried over anhydrous sodium sulfate, and the crude product was concentrated under reduced pressure and purified by column chromatography (eluents: petroleum ether:ethyl acetate=5:1) to obtain the target compound 18D (0.2 g, yield: 43%).

LCMS m/z=323.1 [M+1]⁺

Step 4: Compound 18D (0.2 g, 0.62 mmol) was weighed into a 100 mL single-mouth flask and then dissolved by adding methanol (10 mL), and a hydrogen chloride dioxane solution (4N, 10 mL) was added. After the addition was complete, the mixture was stirred for 2 h. The organic phase was concentrated under reduced pressure until no liquid dripped out to obtain the target compound 18E (0.15 g, crude).

LCMS m/z=223.1 [M+1]⁺

Step 5: Compound 8A-P2 (single configuration, with the configuration being undetermined) (150 mg, 0.50 mmol), compound 18E (110 mg, 0.50 mmol), and N,N-diisopropylethylamine (0.20 g, 1.51 mmol) were added to a 100 mL single-mouth flask. After the addition was complete, the system was placed under nitrogen protection and stirred at 90° C. for 16 h, and the reaction liquid was concentrated under reduced pressure to obtain the crude target compound, which was subjected to preparative HPLC to obtain the title compound 18 (single configuration, with the configuration being undetermined) (30 mg, 12%).

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A with a content of 30-70%; c. flow: 15 mL/min; d. elution time: 20 min; retention time: 10.80 min.

LCMS m/z=488.1 [M+1]⁺

¹H NMR (400 MHZ, DMSO-d6) δ 8.45 (d, 2H), 6.54 (s, 1H), 4.90 (t, 1H), 4.30 (m, 8H), 3.73 (d, 2H), 3.04-3.11 (m, 1H), 2.88-2.95 (m, 1H), 2.58-2.72 (m, 2H), 2.32-2.44 (m, 3H), 2.15 (s, 2H), 1.91-2.00 (m, 1H), 1.71-1.88 (m, 2H).

### Example 20

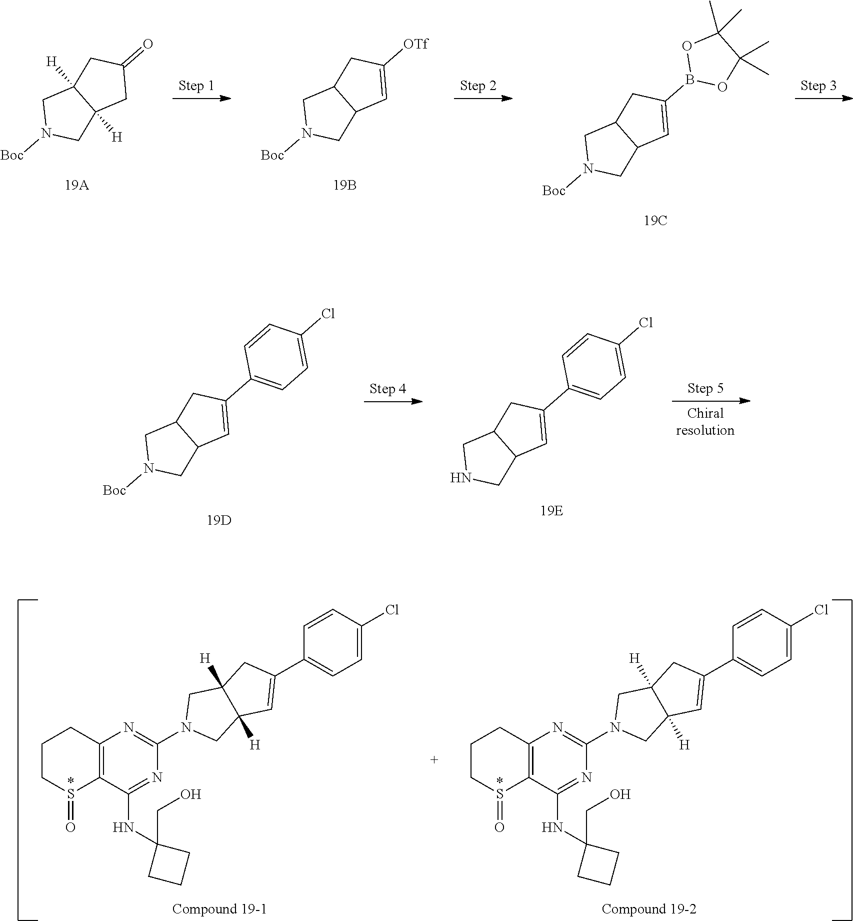

Step 1: Compound 19A (1 g, 4.44 mmol) was dissolved in anhydrous tetrahydrofuran (10 ml). After nitrogen displacement three times, the reaction system was cooled to −78° C., and lithium bis(trimethylsilyl)amide (8.88 mmol) was dropwise added. After the system was maintained at −78° C. for 30 min, N-phenyl-bis(trifluoromethanesulfonimide) (2.59 g, 6.66 mmol) dissolved in anhydrous tetrahydrofuran (5 ml) was dropwise added. The system was further maintained at −78° C. for 30 min, then transferred to room temperature, and reacted for 2 h. After the reaction was complete, the reaction was quenched by adding a saturated ammonium chloride solution (30 ml), the organic phase was extracted with ethyl acetate (3×40 ml), and the organic phases were combined, and dried over anhydrous sodium sulfate. After filtration and concentration, the concentrated product was subjected to quick separation and purification by column chromatography (eluent ratio: EA/PE=0-10%) to obtain compound 19B (1.3 g, 82%).

LCMS (ESI): m/z=302.2 [M+H-56]⁺

Step 2: Under nitrogen, bis(triphenylphosphine)palladium dichloride (260 mg, 0.37 mmol), pinacol diboronate (1.11 g, 4.37 mmol), and potassium acetate (1.07 g, 10.92 mmol) were added to compound 19B (1.3 g, 3.64 mmol) dissolved in 1,4-dioxane (20 ml) and reacted in an oil bath at 90° C. for 18 h. After the reaction was complete, the system was cooled to room temperature, filtered with diatomite, and washed with ethyl acetate, and the filtrate was concentrated and directly subjected to quick separation and purification by column chromatography (eluent ratio: EA/PE=0-10%) to obtain the target compound 19C (0.89 g, 73%).

LCMS (ESI): m/z=280.2 [M+H-56]⁺

Step 3: Under nitrogen, 5-chloro-2-iodobenzene (574 mg, 2.41 mmol), 1,1-bis(diphenylphosphine)ferrocenepalladium dichloride (176 mg, 0.24 mmol), and sodium carbonate (766 mg, 7.32 mmol) were added to compound 19C (0.89 g, 2.65 mmol) dissolved in 1,4-dioxane/water (10 ml/2 ml) and reacted in an oil bath at 90° C. for 18 h. After the reaction was complete, the system was cooled to room temperature, filtered with diatomite, and washed with ethyl acetate, and the filtrate was concentrated and directly subjected to quick separation and purification by column chromatography (eluent ratio: EA/PE=0-10%) to obtain the target compound 19D (0.44 g, 52%).

LCMS (ESI): m/z=264.2 [M+H-56]⁺

Step 4: Compound 19D (220 mg, 0.69 mmol) was dissolved in hydrogen chloride 1,4-dioxane (2 ml), and the mixture was stirred at room temperature for 1 h and directly spin-dried to obtain compound 19E hydrochloride (170 mg).

LCMS (ESI): m/z=220.1 [M+H]⁺

Step 5: Intermediate 8A-P2 (single configuration, with the configuration being undetermined) (100 mg, 0.33 mmol) was dissolved in 1,4-dioxane (5 ml), compound 19E hydrochloride (85 mg, 0.33 mmol) and diisopropylethylamine (128 mg, 0.99 mmol) were added, and the reaction system was placed in an oil bath at 100° C. and reacted for 15 h. After the reaction was complete, the system was cooled to room temperature. The reaction was monitored by LCMS. The system was spin-dried and then subjected to preparation. Preparative HPLC separation method: 1. instrument: waters 2767 preparative liquid chromatographic instrument; chromatographic column: SunFire® Prep C18 (19 mm×250 mm); 2. The sample was filtered with a 0.45 μm filter to prepare a sample liquid. 3. Preparative chromatography conditions: a. composition of mobile phases A and B: mobile phase A: acetonitrile; mobile phase B: water (containing 0.1% ammonium acetate); b. gradient elution, mobile phase A: with a content of 10-55%; c. flow: 12 mL/min. With retention time 7.0 min, compound 19 (130 mg, 81%) was obtained.

LCMS (ESI): m/z=485.3 [M+H]⁺

Resolution of compound 19:

Compound 19 (130 mg) was taken for resolution, and after separation, compound 19-1 (retention time: 1.134 min, 37 mg, ee %=100%) and compound 19-2 (retention time: 1.421 min, 32 mg, ee %=100%) were obtained.

### Resolution Conditions:

instrument: Waters 150 MGM; column: DAICEL CHIRALPAK AD;
mobile phase: A for CO₂ and B for EtOH (0.1% NH₃·H₂O)); gradient: B 40%; flow: 120 mL/min; back pressure: 100 bar;
column temperature: 35° C.; wavelength: 220 nm; period: 13 min;

### Compound 19-1:

¹H NMR (400 MHz, CDCl₃) δ=7.33 (d, 2H), 7.27 (d, 2H), 6.19 (s, 1H), 6.03 (s, 1H), 3.94 (s, 1H), 3.87 (s, 2H), 3.80-3.65 (m, 2H), 3.60 (s, 1H), 3.21-3.16 (m, 2H), 3.11 (s, 1H), 3.00-2.74 (m, 3H), 2.67-2.46 (m, 3H), 2.45-2.26 (m, 2H), 2.24-2.11 (m, 2H), 2.10-2.02 (m, 1H), 2.01-1.87 (m, 2H).

### Compound 19-2:

¹H NMR (400 MHz, CDCl₃) δ=7.32 (d, 2H), 7.27 (d, 2H), 6.18 (s, 1H), 6.03 (s, 1H), 4.00 (s, 1H), 3.87 (s, 2H), 3.74-3.66 (m, 2H), 3.60 (s, 1H), 3.29-3.15 (m, 2H), 3.15-3.03 (m, 1H), 3.02-2.73 (m, 3H), 2.68-2.47 (m, 3H), 2.40 (s, 1H), 2.36-2.26 (m, 1H), 2.23-2.10 (m, 2H), 2.10-2.01 (m, 1H), 2.02-1.84 (m, 2H).

### Example 21

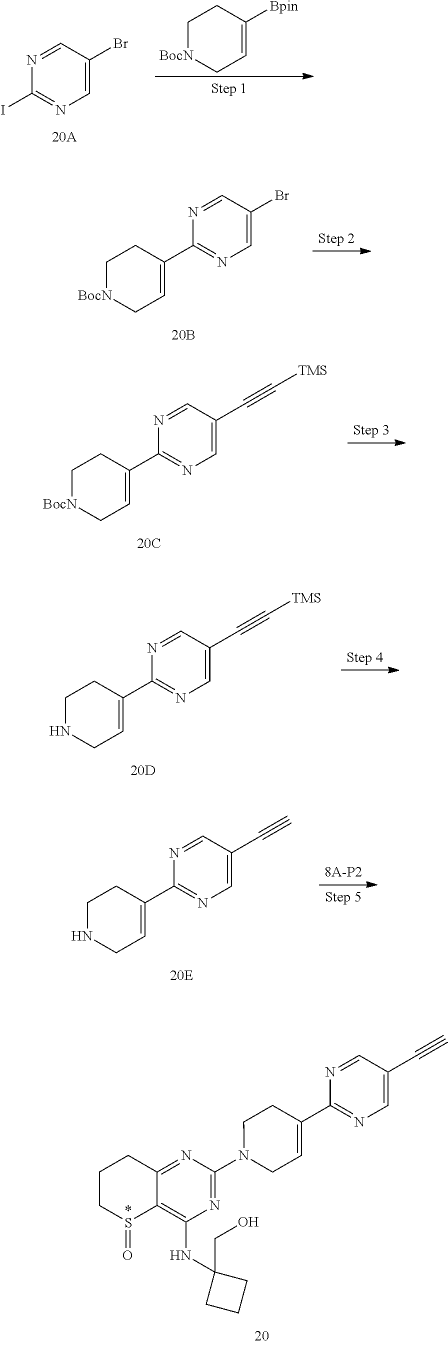

Step 1: Compound 20A (1.4 g, 5.0 mmol) was dissolved in 1,4-dioxane (30 mL), and 1,1-bis(diphenylphosphine)ferrocenepalladium dichloride (360.0 mg, 0.5 mmol), potassium carbonate (1.4 g, 10.0 mmol), N-Boc-1,2,5,6-tetrahydropyridine-4-boronic acid pinacol ester (1.8 g, 6.0 mmol), and water (6.0 mL) were then added. After nitrogen displacement three times, the system was heated to 45° C. and reacted for 14 h. The system passed through a short silica gel column, eluted with ethyl acetate, concentrated, and subjected to column chromatography (dichloromethane/methanol=20/1) to obtain compound 20B (1.7 g, 99%).

LC-MS (ESI): m/z=284.1 [M−56]⁺

Step 2: Compound 20B (340.0 mg, 1.0 mmol) was dissolved in tetrahydrofuran (10 mL), and bis(triphenylphosphine)palladium dichloride (70.0 mg, 0.1 mmol), triethylamine (202.0 mg, 2.0 mmol), trimethylsilylacetylene (130.0 mg, 1.3 mmol), and cuprous iodide (8.0 mg, 0.05 mmol) were then added. After nitrogen displacement three times, the system was heated to 50° C. and reacted for 14 h. The system passed through a short silica gel column, eluted with ethyl acetate, concentrated, and subjected to column chromatography (dichloromethane/methanol=20/1) to obtain compound 20C (240.0 mg, 72%).

LC-MS (ESI): m/z=302.1 [M−56]⁺

Step 3: Compound 20C (240.0 mg, 0.67 mmol) was dissolved in dichloromethane (10 mL), trifluoroacetic acid (1.0 mL) was then added, and the mixture was reacted at room temperature for half an hour and then concentrated to obtain compound 20D (150.0 mg, 87%), which was directly used for the next step.

LC-MS (ESI): m/z=258.1 [M+H]⁺

Step 4: Compound 20D (150.0 mg, 0.58 mmol) was dissolved in methanol (10.0 mL), potassium carbonate (150.0 mg, 1.1 mmol) was then added, and the mixture was reacted at room temperature for 2 h, then concentrated, and subjected to column chromatography (dichloromethane/methanol=15/1) to obtain compound 20E (70.0 mg, 65%).

LC-MS (ESI): m/z=186.1 [M+H]⁺

Step 5: Compound 20E (140.0 mg, 0.74 mmol) was dissolved in 1,4-dioxane (15.0 mL), compound 8A-P2 (single configuration, with the configuration being undetermined) (230.0 mg, 0.8 mmol) was then added, and the mixture was heated back to 90° C., reacted for 12 h, then cooled to room temperature, concentrated, and subjected to column chromatography (dichloromethane/methanol=10/1) to obtain compound 20 (120.0 mg, 62%).

LC-MS (ESI): m/z=451.1 [M+H]⁺

¹H NMR (400 MHz, DMSO-d6) δ 8.90 (s, 2H), 7.34 (s, 1H), 6.53 (s, 1H), 4.89 (s, 1H), 4.68 (s, 1H), 4.45 (s, 2H), 3.95-3.92 (m, 2H), 3.73 (d, 2H), 3.09-3.05 (m, 1H), 2.95-2.88 (m, 1H), 2.66 (d, 4H), 2.44-2.31 (m, 3H), 2.20-2.14 (m, 2H), 1.98-1.93 (m, 1H), 1.87-1.77 (m, 2H).

### Example 22

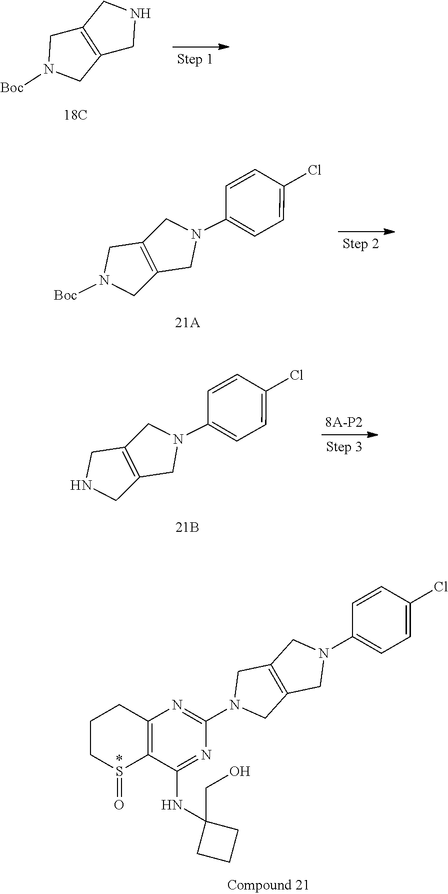

Step 1: Compound 18C (0.50 g, 2.38 mmol) and 1-chloro-4-iodobenzene (0.68 g, 2.68 mmol) were weighed into a 100 mL single-mouth flask and then dissolved by adding 1,4-dioxane (30 mL). 1,1′-Binaphthyl-2,2′-bisphenylphosphine (0.15 g, 0.24 mmol), sodium tert-butoxide (0.69 g, 7.14 mmol), and tris(dibenzylideneacetone)dipalladium (0.14 g, 0.24 mmol) were added to the reaction liquid. After the addition was complete, the mixture was stirred at 85 degrees Celsius for 16 h. After the reaction was complete as determined by a TLC spotting plate (petroleum ether:ethyl acetate=5:1), water (20 mL) was added to the reaction liquid and stirred for 5 min. The liquid was extracted by adding ethyl acetate (20 mL), and the organic phase was separated. The organic phase was dried over anhydrous sodium sulfate, and the crude product was concentrated under reduced pressure and purified by column chromatography (eluents: petroleum ether:ethyl acetate=5:1) to obtain the target compound 21A (0.28 g, yield: 37%).

LCMS m/z=321.1 [M+1]⁺

Step 2: Compound 21A (0.2 g, 0.62 mmol) was weighed into a 100 mL single-mouth flask and then dissolved by adding methanol (10 mL), and a hydrogen chloride dioxane solution (4N, 10 mL) was added. After the addition was complete, the mixture was stirred for 2 h. The organic phase was concentrated under reduced pressure to obtain the target compound 21B (0.15 g, crude).

LCMS m/z=221.1 [M+1]⁺

Step 3: Compound 8A-P2 (150 mg, 0.50 mmol), compound 21B (110 mg, 0.50 mmol), and N,N-diisopropylethylamine (0.20 g, 1.51 mmol) were added to a 100 mL single-mouth flask. After the addition was complete, the system was placed under nitrogen protection and stirred at 90° C. for 16 h, and the reaction liquid was concentrated under reduced pressure to obtain the crude target compound, which was subjected to preparative HPLC to obtain the title compound 21 (50 mg, 20%).

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A with a content of 40-80%; c. flow: 15 mL/min; d. elution time: 20 min; retention time: 12.20 min.

LCMS (ESI): m/z=486.1 [M+1]⁺

¹H NMR (400 MHZ, DMSO-d6) δ 7.21 (d, 2H), 6.53 (d, 2H), 4.90 (t, 1H), 4.31 (s, 4H), 4.10 (s, 4H), 3.73 (d, 2H), 3.05-3.10 (m, 1H), 2.89-2.95 (m, 1H), 2.55-2.72 (m, 2H), 2.32-2.46 (m, 4H), 2.16 (s, 2H), 1.92-2.00 (m, 1H), 1.71-1.87 (m, 2H).

### Example 23

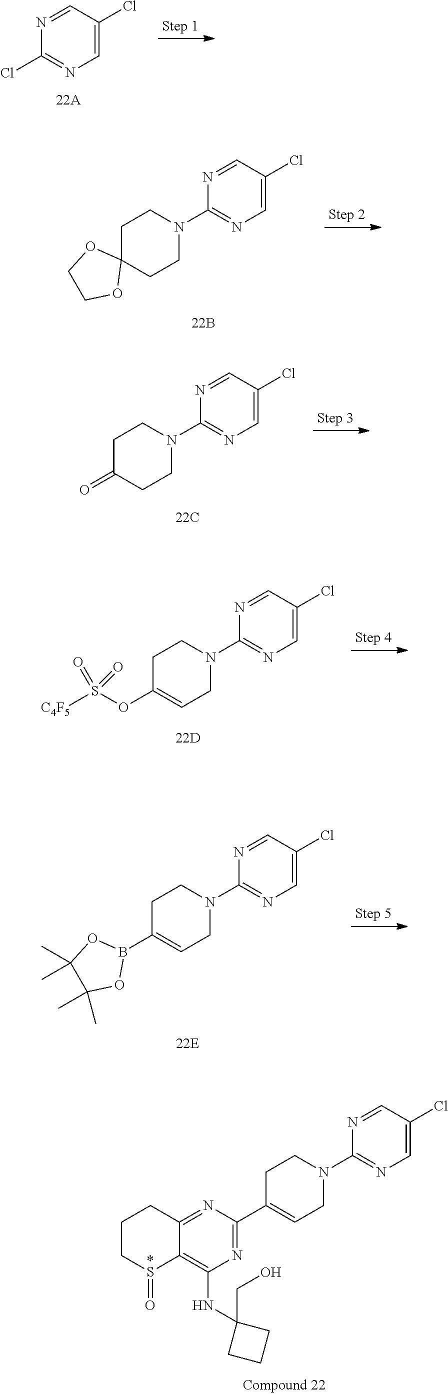

Step 1: 22A (5.0 g, 33.6 mmol), 1,4-dioxa-8-azaspiro[4.5]decane (5.3 g, 36.9 mmol), and DIPEA (13.0 g, 100.8 mmol) were dissolved in 100 mL of dioxane, and the system was heated to 80° C. and reacted for 2 h. The reaction system was poured into ice water and extracted twice with ethyl acetate, the organic phases were combined and dried, and the solvent was removed by rotary evaporation. Purification was carried out by silica gel column chromatography (PE:EA=20:1) to obtain the target compound 22B (8.5 g, yield 99%).

LCMS (ESI): m/z=256.2 [M+H]⁺

Step 2: The raw material 22B (8.5 g, 33.2 mmol) was dissolved in 30 mL of tetrahydrofuran, 120 mL of 10% sulfuric acid was added, and the mixture was heated to 90° C. and reacted under reflux for 12 h. After the reaction was confirmed to be complete by a TLC plotting plate, the system was cooled to room temperature and adjusted to neutral pH with saturated sodium bicarbonate. After washing with ethyl acetate (100 mL×3), the organic phases were washed with a saturated sodium chloride aqueous solution and dried over anhydrous sodium sulfate, and the solvent was removed by rotary evaporation to obtain the target compound 22C (6.2 g, yield 88%), which was directly used in the next reaction.

LCMS (ESI): m/z=212.3 [M+H]⁺

Step 3: The raw material 22C (6.2 g, 29.2 mmol) and 1,1,2,2,3,3,4,4,4-nonafluorobutane-1-sulfonyl fluoride (10.6 g, 35.0 mmol) were dissolved in 62 mL of tetrahydrofuran, 1,8-diazabicyclo[5.4.0]undec-7-ene (5.3 g, 35.0 mmol) was added, and the mixture was stirred at room temperature for 4 h. After the reaction was complete as monitored by TLC, the system was concentrated and then purified by silica gel column chromatography (PE:EA=50:1) to obtain the target compound 22D (10.6 g, yield 73%).

LCMS (ESI): m/z=494.0 [M+H]⁺

Step 4: The raw material 22D (6.50 g, 13.2 mmol), pinacol diboronate (4.02 g, 15.8 mmol), [1,1′-bis(diphenylphosphine)ferrocene]palladium dichloride (190 mg, 0.26 mmol), and 1,1′-bis(diphenylphosphine)ferrocene (144 mg, 0.26 mmol) were suspended in dioxane (65 mL) and reacted at room temperature under stirring for 15 min, and potassium acetate (2.58 g, 26.4 mmol) was added and reacted at 90° C. for 16 h. After the reaction was complete as monitored by TLC and LCMS, purification was carried out by silica gel column chromatography (PE:EA=20:1) to obtain the title compound 22E (3.37 g, yield 79%).

LCMS (ESI): m/z=322.4 [M+H]⁺

Step 5: Compound 22E (500 mg, 1.55 mmol), 8A-P2 (422 mg, 1.40 mmol), tetrakis(triphenylphosphine)palladium (33 mg, 0.03 mmol), and sodium carbonate (493 mg, 4.65 mmol) were suspended in 10 mL of a mixed solvent of dioxane:water=4:1 and reacted at 90° C. for 4 h. After the reaction was complete, the reaction liquid was dropwise added to ice water and filtered. The filtrate was extracted three times with dichloromethane, and the organic phases were combined with the filter residue, concentrated, and purified by silica gel column chromatography (DCM:MeOH=15:1) to obtain the target compound 22 (142 mg, 23%).

¹H NMR (400 MHz, DMSO-d6) δ 8.45 (s, 2H), 7.18 (t, 1H), 6.93 (s, 1H), 4.92 (s, 1H), 4.41-4.35 (m, 2H), 3.97-3.90 (2H), 3.73 (s, 2H), 3.23-3.03 (m, 2H), 2.88-2.77 (m, 1H), 2.75-2.64 (m, 1H), 2.61 (t, 2H), 2.46-2.26 (m, 3H), 2.25-2.15 (m, 2H), 2.11-1.96 (m, 1H), 1.94-1.73 (m, 2H).

LCMS (ESI): m/z=461.19 [M+H]⁺

### Example 24

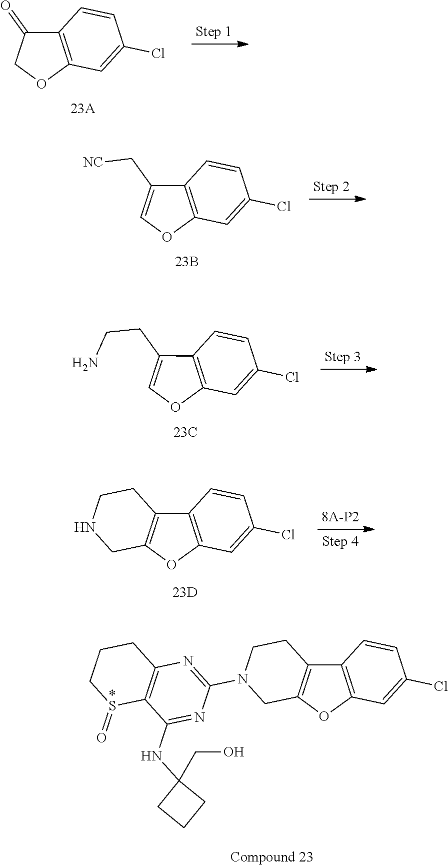

Step 1: Under nitrogen protection, sodium hydride (3.56 g, 88.98 mmol) was slowly added to a solution containing diethyl cyanomethylphosphate (12.61 g, 71.18 mmol) in tetrahydrofuran (100 mL). After the addition was complete, the reaction liquid was stirred at room temperature for 1 h. After the reaction liquid was cooled to −78° C., 23A (10.00 g, 59.32 mmol) was added. After the reaction liquid was reacted under stirring for 2 h, the reaction was quenched by adding water (200 mL), and the system was extracted three times with ethyl acetate (100 mL). The organic phases were combined, dried over anhydrous sodium sulfate, and then concentrated in vacuum to obtain a crude product, and the crude product was purified by column chromatography (eluents: PE:EA=100:1 to 20:1) to obtain the target compound 23B (6.50 g, yield: 57.18%).

¹H NMR (400 MHz, CDCl₃) δ 7.66-7.65 (m, 1H), 7.53-7.52 (m, 1H), 7.51-7.48 (m, 1H), 7.31-7.28 (m, 1H), 3.74 (s, 2H).

Step 2: Compound 23B (6.00 g, 31.31 mmol) was dissolved in methanol (120 mL), raney nickel (1 g) was then added, and the reaction liquid was protected by hydrogen displacement and then stirred at room temperature for 16 h. The reaction liquid was filtered, and the filtrate was concentrated under reduced pressure to obtain a crude product. The crude product was purified by column chromatography (eluents: DCM:MEOH=10:1) to obtain the target compound 23C (1.60 g, yield: 26.12%).

¹H NMR (400 MHz, CDCl₃) δ 7.47-7.46 (m, 1H), 7.42 (s, 1H), 7.40-7.38 (m, 1H), 7.20-7.18 (m, 1H), 2.98-2.92 (m, 2H), 2.85-2.81 (m, 2H).

Step 3: Compound 23C (1.60 g, 8.18 mmol) was dissolved in 30 mL of formic acid, paraformaldehyde (3.20 g) was added, and the mixture was then heated to 70° C. and stirred for 3 h. The reaction liquid was cooled to room temperature, then concentrated, then washed with a sodium hydroxide aqueous solution, and extracted with ethyl acetate. The organic phase was dried and concentrated to obtain a crude product, and the crude product was purified by column chromatography (eluents: DCM:MEOH=20:1-10:1) to obtain the target compound 23D (250 mg, yield: 14.72%).

LCMS m/z=208.1 [M+H]⁺

Step 4: Compound 8A-P2 (200 mg, 0.66 mmol) and compound 23D (250 mg, 1.20 mmol) were dissolved in 1,4-dioxane (6 mL), and DIPEA (235 mg, 1.82 mmol) was then added. After the addition was complete, the mixture was heated to 85° C. and stirred for 16 h under nitrogen protection. After the reaction was complete, the reaction liquid was concentrated under reduced pressure to obtain a crude product, which was purified by column chromatography (eluents: DCM:MeOH=100:1 to 9:1) and then further purified by prep.HPLC (preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A with a content of 25-85%; c. flow: 15 mL/min; d. elution time: 15 min; retention time: 7.0 min) to obtain the target compound 23 (82.5 mg, 26.32%).

LCMS m/z=473.2 [M+H]⁺

¹H NMR (400 MHz, CDCl₃) δ 7.45-7.44 (m, 1H), 7.34-7.32 (m, 1H), 7.22-7.19 (m, 1H), 6.23 (br s, 1H), 4.90 (s, 2H), 4.11 (s, 2H), 3.94 (s, 2H), 3.23-3.18 (m, 1H), 2.93-2.85 (m, 2H), 2.75 (s, 2H), 2.64-2.49 (m, 2H), 2.42-2.33 (m, 2H), 2.30-2.22 (m, 2H), 2.11-1.90 (m, 4H).

### Example 25

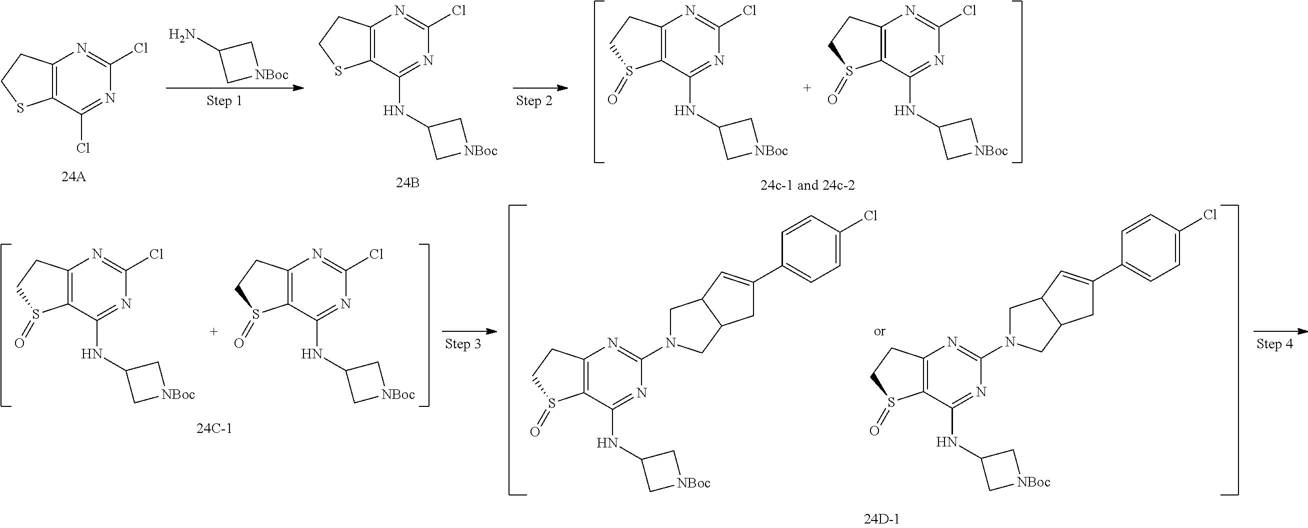

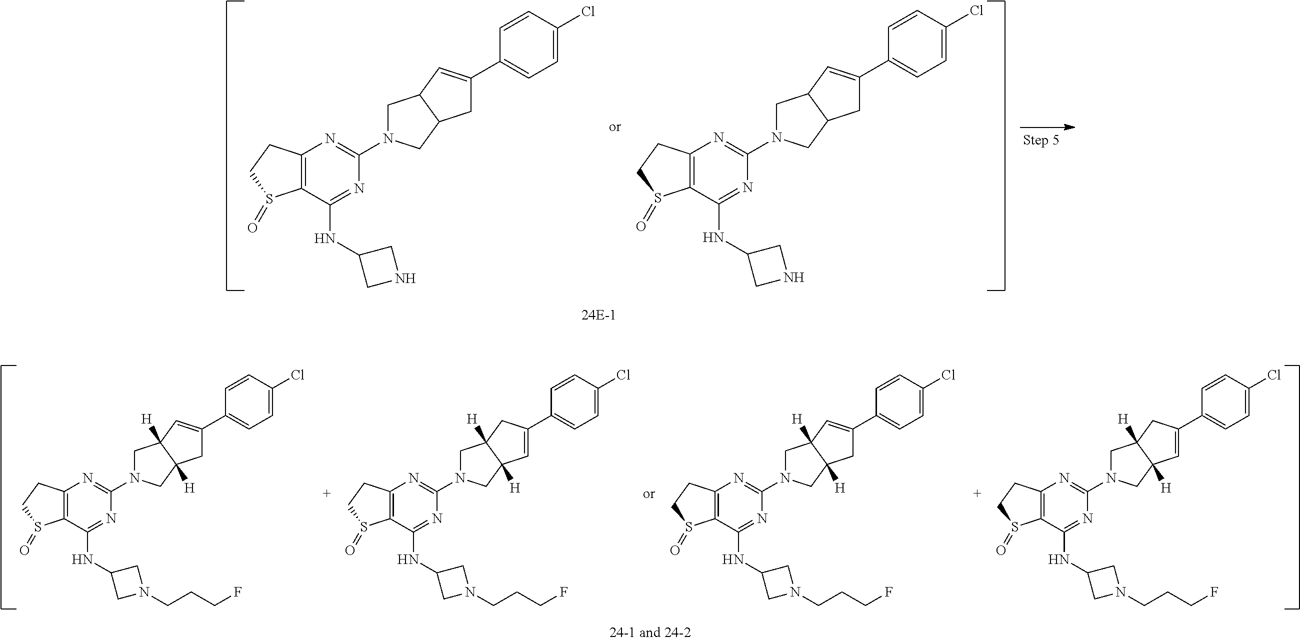

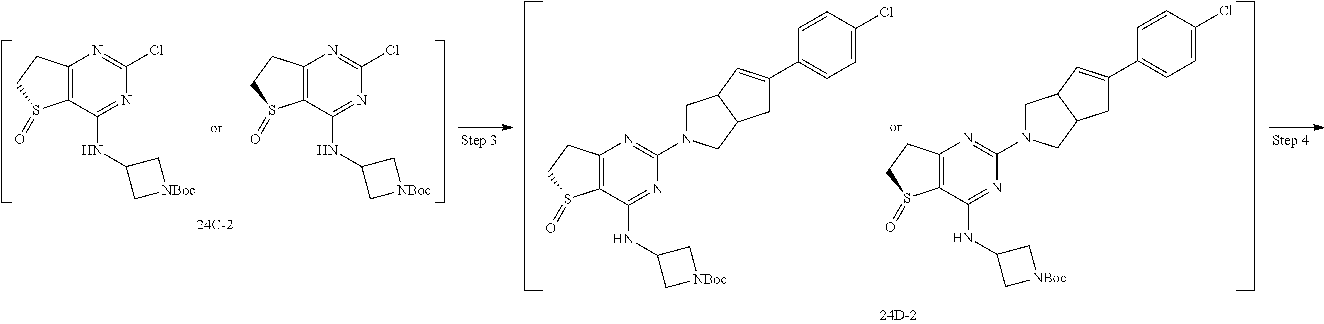

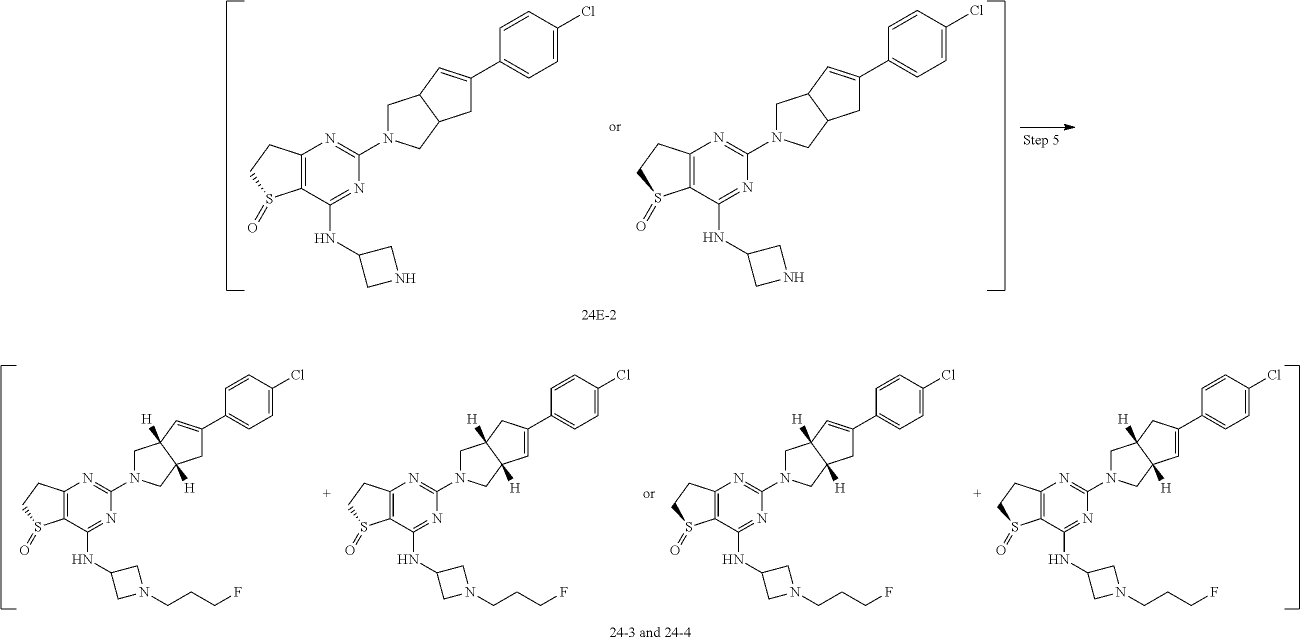

Step 1: 24A (5 g, 24.18 mmol), 1-tert-butoxycarbonyl-3-aminocyclobutylamine (5 g, 29.02 mmol), and N,N-diisopropylethylamine (9.38 g, 72.54 mmol) were added to acetonitrile (50 mL), heated to 60° C., and reacted for 16 h. After the reaction was complete, the system was cooled to room temperature, the reaction liquid was concentrated under reduced pressure to remove most acetonitrile, and water (50 ml) and ethyl acetate (50 ml) were then added. After liquid separation, the aqueous phase was extracted with ethyl acetate (50 ml×2), the organic phases were combined, washed with a saturated sodium chloride solution, and dried over anhydrous sodium sulfate. After filtration, the filtrate was concentrated under reduced pressure and then separated and purified by silica gel column chromatography (eluents EA/PE=0-60%) to obtain the title compound 24B (7 g, 84.44%).

LC-MS (ESI): m/z=343.1 [M+H]⁺.

Step 2: 24B (7 g, 20.42 mmol), tetraisopropyl titanate (0.58 g, 2.04 mmol), and water (0.37 g, 20.42 mmol) were successively added to dichloromethane (100 mL) and stirred for 10 min, tert-butyl hydroperoxide (7.89 g, 61.26 mmol, purity 70%) was then added, and the mixture was reacted at room temperature for 5 h. As monitored by TLC, the reaction was complete. Water (50 ml) was added to the reaction liquid. After liquid separation, the organic phase was washed with a sodium thiosulfate aqueous solution (50 ml), then washed with a saturated sodium chloride solution, and dried over anhydrous sodium sulfate. After filtration, the filtrate was concentrated under reduced pressure and then separated and purified by silica gel column chromatography (eluents EA/PE=0-100%) to obtain compound 24C (6 g, 82.6%).

24C (5 g) was taken and subjected to chiral resolution. Chiral preparation was carried out to obtain the title compound 24C-1 (2.2 g, 44%, retention time: 0.853 min) and the title compound 24C-2 (1.6 g, 32%, retention time: 1.070 min). Chiral preparation method: instrument: Waters 150 SFC; chromatographic column: Chiralpak AS Column; mobile phase: A: carbon dioxide, and B: methanol (0.1% aqueous ammonia); isocratic elution: 30% mobile phase B; flow rate: 120 mL/min; back pressure: 100 bar; column temperature: 25° C.; wavelength: 220 nm; elution time: 2.5 min.

LC-MS (ESI): m/z=359.1 [M+H]⁺.

Step 3: 24C-1 (1 g, 2.79 mmol), 13C (1.12 g, 3.35 mmol), and N,N-diisopropylethylamine (1.80 g, 13.92 mmol) were added to 1,4-dioxane (15 mL), heated to 100° C., and reacted for 4 h. After the reaction was complete as monitored by LC-MS, the system was cooled to room temperature, directly concentrated under reduced pressure, and then separated and purified by silica gel column chromatography (eluents MeOH/DCM=0-10%) to obtain the title compound 24D-1 (1.2 g, 79.34%).

According to the above operation, 24D-2 (1.2 g, 79.34%) was obtained from 24C-2 (1 g, 2.79 mmol) as a raw material.

LC-MS (ESI): m/z=542.2 [M+H]⁺.

Step 4: 24D-1 (1.2 g, 2.21 mmol) was added to dichloromethane (15 mL), trifluoroacetic acid (4 ml) was then added, and the mixture was reacted at room temperature for 1 h. The reaction liquid was directly concentrated under reduced pressure to obtain crude 24E-1 (1.7 g).

According to the above operation, 24E-2 (1.7 g) was obtained from 24D-2 (1.2 g, 2.21 mmol) as a raw material.

LC-MS (ESI): m/z=442.2 [M+H]⁺.

Step 5: Crude product 24E-1 (1.7 g) and 3-fluoro-1-iodopropane (0.61 g, 3.25 mmol) were added to tetrahydrofuran (20 mL), a sodium hydroxide (0.52 g, 13 mmol) aqueous solution (5 mol/L) was then added, and the mixture was reacted at room temperature for 16 h. The reaction liquid was concentrated under reduced pressure to remove tetrahydrofuran, water (20 ml) was added, and the mixture was extracted with (MeOH/DCM=1/10) (30 ml×3). The organic phases were combined, washed with a saturated sodium chloride solution, and dried over anhydrous sodium sulfate. After filtration, the filtrate was concentrated under reduced pressure and then separated and purified by silica gel column chromatography (MeOH/DCM=0-15%) to obtain a product (0.6 g, 55%).

Chiral preparation was carried out to obtain the title compound 24-1 (200 mg, 18.36%, retention time: 1.525 min) and the title compound 24-2 (230 mg, 21.11%, retention time: 2.022 min). Chiral preparation method: instrument: Waters 150 preparative SFC (SFC-26); chromatographic column: ChiralPak AD, 250×30 mm I.D., 10 μm; mobile phase: A: carbon dioxide, and B: isopropanol (0.1% aqueous ammonia); isocratic elution: 30% mobile phase B; flow rate: 150 mL/min; back pressure: 100 bar; column temperature: 38° C.; wavelength: 220 nm; elution time: 15 min.

According to the above operation, using 24E-2 (1.7 g) as a raw material, a compound (0.7 g, 64%) was obtained, which was subjected to chiral separation to obtain 24-3 and 24-4.

Chiral preparation was carried out to obtain the title compound 24-3 (310 mg, 28.45%, retention time: 0.698 min) and the title compound 24-4 (280 mg, 25.7%, retention time: 1.514 min). Chiral preparation method: instrument: Waters 150 preparative SFC (SFC-26); chromatographic column: ChiralPak AD, 250×30 mm I.D., 10 μm; mobile phase: A: carbon dioxide, and B: ethanol (0.1% aqueous ammonia); isocratic elution: 65% mobile phase B; flow rate: 100 mL/min; back pressure: 100 bar; column temperature: 38° C.; wavelength: 220 nm; elution time: 5 min.

### (Compound 24-1)

¹H NMR (400 MHz, DMSO-d6) δ 8.01 (s, 1H), 7.51-7.47 (m, 2H), 7.41-7.37 (m, 2H), 6.26 (s, 1H), 4.57-4.50 (m, 2H), 4.42-4.38 (m, 1H), 3.88 (dd, 1H), 3.80-3.72 (m, 1H), 3.69-3.52 (m, 4H), 3.46-3.38 (m, 1H), 3.25-3.15 (m, 2H), 3.12-3.06 (m, 1H), 3.02-2.82 (m, 5H), 2.69-2.62 (m, 1H), 2.56-2.50 (m, 2H), 1.72-1.60 (m, 2H).

LC-MS (ESI): m/z=502.2 [M+H]⁺.

### (Compound 24-2)

¹H NMR (400 MHz, DMSO-d6) δ 8.00 (s, 1H), 7.51-7.47 (m, 2H), 7.41-7.37 (m, 2H), 6.26 (s, 1H), 4.56-4.49 (m, 2H), 4.42-4.37 (m, 1H), 3.88 (dd, 1H), 3.80-3.71 (m, 1H), 3.68-3.52 (m, 4H), 3.47-3.35 (m, 1H), 3.24-3.14 (m, 2H), 3.12-3.04 (m, 1H), 3.00-2.81 (m, 5H), 2.71-2.59 (m, 1H), 2.49-2.43 (m, 2H), 1.72-1.58 (m, 2H).

LC-MS (ESI): m/z=502.2 [M+H]⁺.

### (Compound 24-3)

¹H NMR (400 MHz, CDCl₃) δ 7.38-7.26 (m, 4H), 6.58-6.52 (m, 1H), 6.05 (s, 1H), 4.77-4.68 (m, 1H), 4.55-4.47 (m, 1H), 4.43-4.36 (m, 1H), 4.03-3.93 (m, 1H), 3.83-3.69 (m, 3H), 3.68-3.52 (m, 3H), 3.38-3.21 (m, 2H), 3.19-3.08 (m, 1H), 3.08-2.94 (m, 3H), 2.93-2.87 (m, 1H), 2.82-2.69 (m, 1H), 2.66-2.57 (m, 1H), 2.56-2.40 (m, 2H), 1.78-1.62 (m, 2H).

LC-MS (ESI): m/z=502.2 [M+H]⁺.

### (Compound 24-4)

¹H NMR (400 MHz, CDCl₃) δ 7.38-7.26 (m, 4H), 6.65-6.32 (m, 1H), 6.05 (s, 1H), 4.83-4.70 (m, 1H), 4.55-4.46 (m, 1H), 4.43-4.34 (m, 1H), 4.03-3.92 (m, 1H), 3.87-3.66 (m, 4H), 3.66-3.52 (m, 2H), 3.39-3.23 (m, 2H), 3.18-3.08 (m, 1H), 3.07-2.74 (m, 5H), 2.68-2.59 (m, 1H), 2.58-2.44 (m, 2H), 1.84-1.71 (m, 2H).

LC-MS (ESI): m/z=502.2 [M+H]⁺.

### Example 26

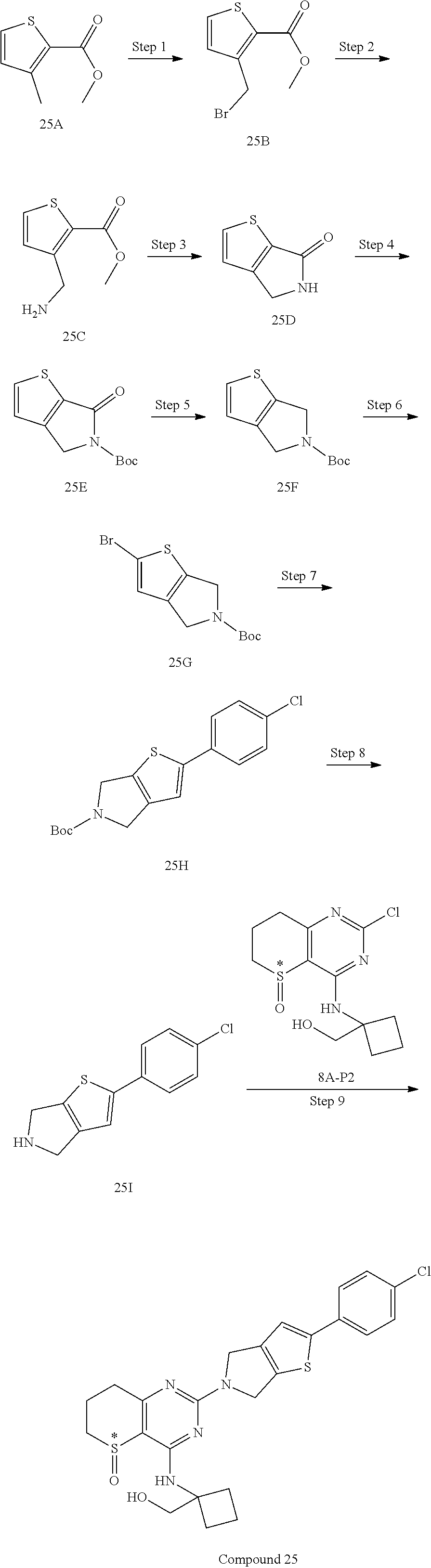

Step 1: Compound 25A (15 g, 96.03 mmol) was dissolved in carbon tetrachloride (600 mL), and N-bromosuccinimide (17.09 g, 96.03 mmol) and azodiisobutyronitrile (1.58 g, 9.60 mmol) were added thereto. Under nitrogen protection, the mixture was heated to 70° C. and reacted under stirring overnight, and the system was cooled to room temperature, washed with water and a saturated sodium chloride aqueous solution, and dried over anhydrous sodium sulfate. Then, the filtrate was spin-dried to obtain compound 25B (24 g, crude), which was directly used in the next reaction without separation and purification.

LCMS (ESI): =235.1, 237.1 [M+H]⁺.

Step 2: Crude 25B (17 g, 63.8 mmol) was dissolved in an ammonia methanol solution (7.0 M, 20 mL) and methanol (20 mL) and stirred at room temperature for 2 hours. The solvent was evaporated under reduced pressure, and the remaining material was then separated by a silica gel chromatographic column (DCM:MeOH, v/v=10:1) to obtain compound 25C (8 g, two-step yield 48.7%).

LCMS (ESI): =172.1 [M+H]⁺.

Step 3: Compound 25C (8 g, 46.73 mmol) was dissolved in a mixed solvent of methanol and ethanol (120 mL, v/v=1:1), potassium carbonate (12.92 g, 93.45 mmol) was added thereto, and the mixture was stirred, heated to 90° C., and reacted for 3 hours. The system was cooled to room temperature, the solvent was evaporated under reduced pressure, and 150 ml of ethyl acetate was then added for dissolution. Then, a solid was filtered off, and the solvent was spin-dried to obtain compound 25D (3.25 g, 36%).

LCMS (ESI): =140.0 [M+H]⁺.

Step 4: 25D (3.25 g, 23.35 mmol) was dissolved in dichloromethane (50 mL), triethylamine (7.09 g, 70.06 mmol) and a catalytic amount of 4-dimethylaminopyridine (285 mg, 2.34 mmol) were added thereto, and the mixture was cooled in an ice bath. Di-tert-butyl dicarbonate (10.2 g, 46.71 mmol) was dropwise added, and the mixture was stirred at room temperature for 3 hours, directly spin-dried, and separated by a silica gel chromatographic column (EA:PE=1:3) to obtain compound 25E (4.45 g, 79.6%).

LCMS (ESI): =184.1 [M+H]⁺.

Step 5: 25E (4.45 g, 18.60 mmol) was dissolved in tetrahydrofuran (50 mL). Under nitrogen protection and cooling with ice water, borane tetrahydrofuran complex (55.8 mL, 1.0 mol/L) was dropwise added thereto. After the dropwise addition was complete, the mixture was reacted at room temperature for 3 hours. After cooling with ice water, the reaction was quenched with a small amount of methanol, and the solvent was evaporated under reduced pressure to obtain a crude product, which was separated by a silica gel chromatographic column (EA:PE=1:3) to obtain compound 25F (2.26 g, 53.9%).

LCMS (ESI): =170.1 [M+H]⁺.

Step 6: 25F (2.26 g, 10.03 mmol) was dissolved in chloroform (25 mL), and a catalytic amount of glacial acetic acid was added. Liquid bromine (1.6 g, 10.03 mmol) was added dropwise under cooling with ice water, the mixture was stirred at room temperature overnight, and triethylamine (3.04 g, 30.09 mmol) and a catalytic amount of DMAP (123 mg, 1.00 mmol) were added thereto. The mixture was cooled in an ice bath, di-tert-butyl dicarbonate (4.38 g, 20.06 mmol) was dropwise added, and the mixture was stirred at room temperature for 3 hours, directly spin-dried, and separated by a silica gel chromatographic column (EA:PE=1:3) to obtain compound 25G (2.26 g, 74.1%).

LCMS (ESI): =248.1, 250.1 [M+H]⁺.

Step 7: 25G (1 g, 3.29 mmol), 4-chlorophenylboronic acid (616.8 mg, 3.94 mmol), sodium carbonate (697 mg, 6.57 mmol), and tetrakis(triphenylphosphine)palladium (380 mg, 0.33 mmol) were added to a mixed solution of 1,4-dioxane and water (27.5 mL, v/v=10:1). After nitrogen displacement, the mixture was reacted at 80° C. for 3 hours. The reaction was quenched by adding water, the system was extracted with ethyl acetate, washed with a saturated sodium chloride aqueous solution, dried over anhydrous sodium sulfate, and separated by a silica gel chromatographic column to obtain compound 25H (640 mg, 58.0%).

LCMS (ESI): =280.1, 282.1 [M+H]⁺.

Step 8: 25H (300 mg) was added to hydrogen chloride dioxane (5 mL, 4N), and the mixture was reacted at room temperature for 2 hours. Then, the system was spin-dried to obtain compound 251 (300 mg, crude), which was directly used in the next reaction.

LCMS (ESI): =236.1, 238.1 [M+H]⁺.

Step 9: 8A-P2 (150 mg, 0.50 mmol), 251 (200 mg, crude), and DIPEA (321 mg, 2.49 mmol) were added to 1,4-dioxane (5 mL), and the mixture was reacted at 80° C. for 3 hours. The solvent was spin-dried, and the remaining material was subjected to HPLC preparation. The prepared liquid was adjusted to pH 7-8 by adding sodium bicarbonate, extracted with dichloromethane, and then concentrated to obtain compound 25 (65 mg, 26.1%).

Preparative HPLC separation method: 1. instrument: waters 2767 preparative liquid chromatographic instrument; chromatographic column: SunFire® Prep C18 (19 mm×250 mm) 2. The sample was filtered with a 0.45 μm filter to prepare a sample liquid. 3. Preparative chromatography conditions: a. composition of mobile phases A and B: mobile phase A: acetonitrile; mobile phase B: water (containing 0.1% TFA); b. gradient elution, mobile phase A: with a content of 5-50%; c. flow: 12 mL/min; d. elution time: 20 min.

LCMS (ESI): =502.1 [M+H]⁺.

¹H NMR (400 MHz, Chloroform-d) δ 7.52-7.45 (m, 2H), 7.37-7.32 (m, 2H), 7.07 (s, 1H), 6.27 (s, 1H), 5.18 (s, 1H), 4.95-4.61 (m, 4H), 4.00-3.87 (m, 2H), 3.28-3.17 (m, 1H), 3.02-2.83 (m, 2H), 2.72-2.49 (m, 2H), 2.47-2.31 (m, 2H), 2.30-2.16 (m, 2H), 2.16-2.06 (m, 1H), 2.05-1.88 (m, 2H).

### Example 27

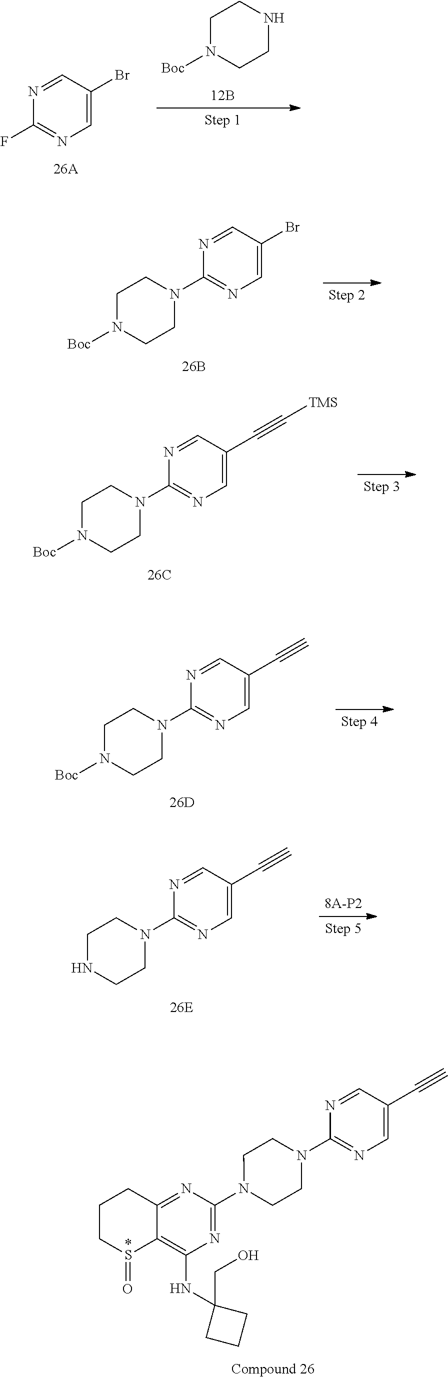

Step 1: Compound 26A (3.00 g, 16.92 mmol) and compound 12B (3.78 g, 20.30 mmol) were weighed into a 100 mL single-mouth flask and then dissolved by adding 1,4-dioxane (30 mL), and N,N-diisopropylethylamine (6.56 g, 50.76 mmol) was added to the reaction liquid. After the addition was complete, the mixture was stirred at 80 degrees Celsius for 16 h. After the reaction was complete as determined by a TLC spotting plate (petroleum ether:ethyl acetate=5:1), water (20 mL) was added to the reaction liquid and stirred for 5 min. The liquid was extracted by adding ethyl acetate (20 mL), and the organic phase was separated. The organic phase was dried over anhydrous sodium sulfate, and the crude product was concentrated under reduced pressure and purified by column chromatography (eluents: petroleum ether:ethyl acetate=5:1) to obtain the target compound 26B (2.8 g, yield: 48%).

LCMS m/z=343.1 [M+1]⁺

Using compound 26B and trimethylsilylacetylene as raw materials, compound 26 (80 mg, 27%) was obtained according to the operation of Example 21.

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A with a content of 35-80%; c. flow: 15 mL/min; d. elution time: 20 min; retention time: 12.50 min.

LCMS m/z=454.2 [M+1]⁺

¹H NMR (400 MHZ, DMSO-d6) δ 8.50 (s, 2H), 6.56 (s, 1H), 4.89 (t, 1H), 4.28 (s, 1H), 3.81 (s, 8H), 3.70 (d, 2H), 3.03-3.11 (m, 1H), 2.88-2.95 (m, 1H), 2.53-2.70 (m, 2H), 2.31-2.45 (m, 3H), 2.15 (s, 2H), 1.91-2.00 (m, 1H), 1.73-1.87 (m, 2H).

### Example 28

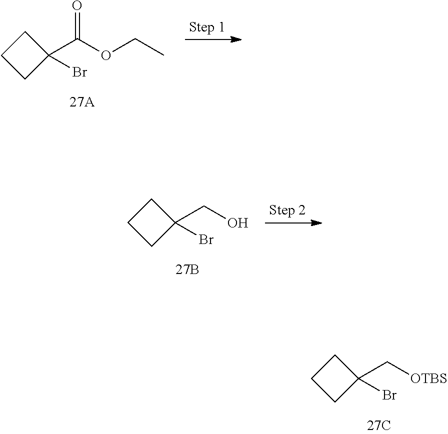

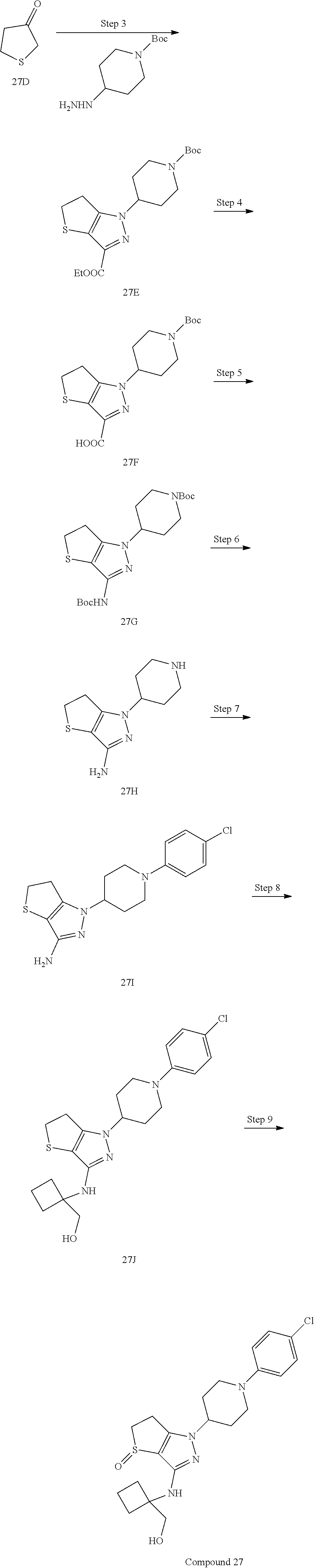

Step 1: The raw material 27A (3 g, 14.49 mmol) was added to 60 mL of a dry tetrahydrofuran solvent, and the reagent lithium tetrahydroaluminate (270 mg, 7.25 mmol) was added in an ice bath. After stirring in the ice bath for half an hour, the mixture was heated to room temperature and stirred for 3 hours. After quenching by adding water, a small amount of dilute hydrochloric acid was added, and the system was then was extracted three times with ethyl acetate, washed with a saturated sodium bicarbonate solution, and dried over anhydrous sodium sulfate. After filtration, the filtrate was concentrated and then directly used for the next reaction.

Step 2: The raw material 27B (2 g, 12.12 mmol) was dissolved in the solvent dichloromethane (50 mL), and the reagents triethylamine (2.45 g, 24.24 mmol), imidazole (1.65 g, 24.24 mmol), and tert-butyldimethylsilyl chloride (2.74 g, 18.18 mmol) were then added. The mixture was stirred at room temperature overnight, diluted by adding water, then extracted with dichloromethane, washed with a saturated sodium bicarbonate solution, and dried over anhydrous sodium sulfate. After filtration, the filtrate was concentrated and then purified and separated by silica gel column chromatography (PE:EA (v/v)=1:0-20:1) to obtain 27C (1.8 g, yield 53%).

¹H NMR (400 MHz, CDCl₃) δ3.68 (s, 2H), 2.45-2.50 (m, 4H), 2.01-2.06 (m, 1H), 1.72-1.79 (m, 1H), 0.83 (s, 9H), 0.0 (s, 6H).

Step 3: The raw material 3-thienone (8 g, 78.28 mmol) and diethyl oxalate (11.44 g, 78.28 mmol) were added to an anhydrous ethanol solvent (70 mL), and the reagent sodium tert-butoxide (7.9 g, 82.21 mmol) was added in an ice bath and further stirred for 2 hours. Glacial acetic acid (5.17 g, 86.11 mmol) was then added, followed by tert-butyl 4-hydrazinopiperidine-1-carboxylate hydrochloride (16.85 g, 78.28 mmol), and the mixture was further stirred for 20 hours. After quenching by adding water, the system was extracted with EA, washed with a saturated sodium bicarbonate solution, and dried over anhydrous sodium sulfate. After filtration, the filtrate was concentrated and then purified and separated by silica gel column chromatography (PE:EA (v/v)=1:0-10:1) to obtain 27E (8 g, yield 27%).

LCMS m/z=382.2 [M+1]⁺

Step 4: The raw material 27E (6 g, 15.73 mmol) was added to a mixed solvent of tetrahydrofuran (30 mL) and methanol (30 mL), and the reagent lithium hydroxide (0.76 g, 31.65 mmol) dissolved in 15 mL of a water solvent was then added and stirred at room temperature for 2 hours. After the raw material was completely reacted as monitored by TLC, the system was concentrated, then diluted by adding water, adjusted to pH 2-3 with 2M hydrochloric acid, extracted with ethyl acetate, and dried over anhydrous sodium sulfate. After filtration, the filtrate was concentrated and directly used for the next reaction.

LCMS m/z=354.1 [M+1]⁺

Step 5: The raw material 27F (4 g, 11.32 mmol) and diphenyl phosphorazidate (5.67 g, 14.72 mmol) were added to tert-butyl alcohol (100 mL), followed by triethylamine (1.72 g, 16.98 mmol). Under nitrogen protection, the mixture was heated to 85° C., stirred for 4 hours, cooled, concentrated, then diluted by adding water, and extracted with ethyl acetate. After drying over anhydrous sodium sulfate and filtration, the filtrate was concentrated and then purified and separated by silica gel column chromatography (PE:EA (v/v)=1:0-4: 1) to obtain the target compound 27G (4 g, yield 83%).

LCMS m/z=425.2 [M+1]⁺

¹H NMR (400 MHz, CDCl₃) δ5.99 (s, 1H), 4.24-4.26 (m, 2H), 4.04-4.06 (m, 1H), 3.94-3.95 (m, 2H), 3.82-3.83 (m, 2H), 2.77-2.81 (m, 2H), 2.07-2.08 (m, 1H), 2.01-2.02 (m, 1H), 1.86-1.89 (m, 2H), 1.54 (s, 9H), 1.48 (s, 9H).

Step 6: The raw material 27G (4 g, 9.42 mmol) was added to anhydrous methanol (5 mL), followed by 20 mL of a hydrogen chloride 1,4-dioxane solution (4M), and the mixture was stirred at room temperature for 4 hours. After the raw material was completely reacted as monitored by LCMS, the system was concentrated to dryness and used for the next reaction.

LCMS m/z=225.1 [M+1]⁺

Step 7: The raw material 27H (2.2 g, 9.81 mmol), 4-chloroiodobenzene (2.34 g, 9.81 mmol), tris(dibenzylideneacetone)palladium (0.45 g, 0.49 mmol), 4,5-bis(diphenylphosphino)-9,9-dimethylxanthene (0.57 g, 0.98 mmol), and sodium tert-butoxide (1.89 g, 19.62 mmol) were separately added to 1,4-dioxane (100 mL). Under nitrogen protection, the mixture was heated to 85° C., stirred for 16 hours, cooled, and filtered, and the filtrate was diluted by adding water, extracted with ethyl acetate, and dried over anhydrous sodium sulfate. After filtration, the filtrate was concentrated and then purified and separated by silica gel column chromatography (PE:EA (v/v)=1:0-4: 1) to obtain the target compound 271 (0.8 g, yield 24%).

LCMS m/z=335.1 [M+1]⁺

Step 8: The raw material 271 (0.7 g, 2.09 mmol) was dissolved in N,N-dimethylacetamide (20 mL), and sodium hydride (0.42 g, 10.45 mmol, 40% in oil) was added in an ice bath and stirred in the ice bath for 1 hour. The reagent 27C (1.17 g, 4.18 mmol) was then added and stirred in the ice bath for half an hour, and the mixture was then heated to room temperature and stirred for 3 hours. After the raw material was completely reacted as monitored by LCMS, the reaction was quenched by adding water, and the system was concentrated under reduced pressure by an oil pump to remove the solvent and then separated by silica gel column chromatography (DCM:MeOH (v/v)=1:0-5:1) to obtain the target compound 27J (0.8 g, yield 91%).

LCMS m/z=419.2 [M+1]⁺

Step 9: The raw material 27J (200 mg, 0.48 mmol), 1,1′-bi-2-naphthol (69 mg, 0.24 mmol), water (9 mg, 0.48 mmol), and tetraisopropyl titanate (68 mg, 0.24 mmol) were successively added to the solvent dichloromethane (10 mL) and stirred at room temperature for half an hour. Tert-butyl hydroperoxide (65 mg, 0.72 mmol) was then added, and the mixture was further stirred for 3 hours, then diluted by adding water, and then extracted with dichloromethane. After washing with saturated sodium bicarbonate followed by drying and filtration, the filtrate was concentrated and then purified and separated by TLC (DCM:MeOH=5:1) to obtain the title compound 27 (0.01 g, yield 5%).

LCMS m/z=435.2 [M+1]⁺

¹H NMR (400 MHz, CD₃OD-d₄) δ 7.14-7.17 (m, 2H), 6.76-6.80 (m, 2H), 4.29-4.39 (m, 2H), 4.15-4.18 (m, 1H), 3.65-3.78 (m, 4H), 3.12-3.15 (m, 2H), 2.52-2.61 (m, 2H), 1.91-2.03 (m, 6H), 1.74-1.81 (m, 3H), 1.60-1.66 (m, 1H).

### Example 29

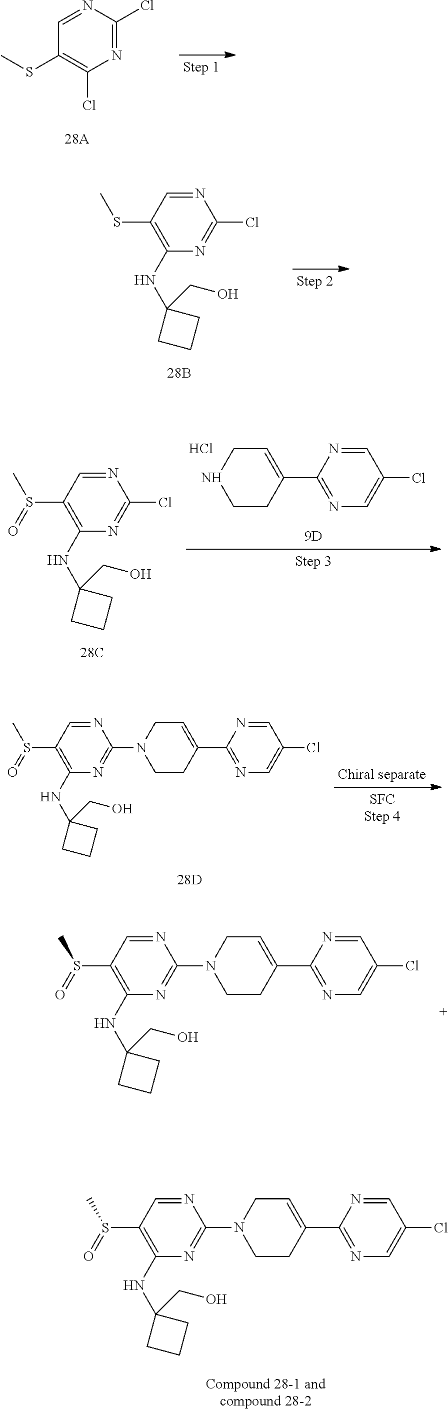

Step 1: Compound 28A (3.9 g, 20.0 mmol) and 1-aminocyclobutylmethanol hydrochloride (3.5 g, 25.0 mmol) were dissolved in acetonitrile (50.0 mL), triethylamine (6.1 g, 60.0 mmol) was then added, and the mixture was heated to 70° C., and reacted for 14 h. The system was then concentrated and subjected to column chromatography (dichloromethane/methanol=10/1) to obtain compound 28B (5.2 g, 98%).

LC-MS (ESI): m/z=260.0 [M+H]⁺

Step 2: Compound 28B (2.0 g, 7.7 mmol) was dissolved in dichloromethane (40 mL), m-chloroperoxybenzoic acid (1.5 g, 8.5 mmol) was added in an ice bath, and the mixture was slowly heated to room temperature and reacted overnight. The system was concentrated to obtain compound 28C (1.7 g, 80%), which was directly used for the next step.

LC-MS (ESI): m/z=276.0 [M+H]⁺

Step 3: Compound 28C (0.7 g, 2.4 mmol) was dissolved in 1,4-dioxane (40.0 mL), compound 9D (461.0 mg, 2.4 mmol) and diisopropylethylamine (1.3 g, 9.4 mmol) were then added, and the mixture was heated back to 90° C. and reacted for 12 h. The system was then cooled to room temperature, concentrated, and subjected to column chromatography (dichloromethane/methanol=9/1) to obtain compound 28D (380.0 mg, 37%).

LC-MS (ESI): m/z=435.1 [M+H]⁺

Step 4: Compound 28D (380 mg) was subjected to chiral resolution to obtain compound 28-1 (100 mg) and compound 28-2 (101 mg).

Preparation method: instrument: MG II preparative SFC (SFC-14), column: ChiralPak AD, 250×30 mm I.D., 5 μm, mobile phase: (A for CO₂ and B for MeOH (0.1% NH₃·H₂O); gradient: 50% phase B elution; flow rate: 100 mL/min, back pressure: 100 bar, column temperature: 25° C.; wavelength: 220 nm; cycle time: 3.3 min; sample preparation: sample concentration 10 mg/mL, acetonitrile solution injection: 3.5 mL each time. After separation, the fraction was dried on a rotary evaporator at a bath temperature of 35° C. to obtain P1 (retention time: 1.334 minutes, assigned as compound 28-1) and P2 (retention time: 1.840 minutes, assigned as compound 28-2).

LC-MS (ESI): m/z=435.6 [M+H]⁺

¹H NMR (400 MHz, DMSO-d6) δ 8.90 (s, 2H), 7.96 (s, 1H), 7.37 (s, 1H), 7.29 (s, 1H), 4.92-4.89 (m, 1H), 4.43 (s, 2H), 3.95 (s, 2H), 3.83-3.79 (m, 1H), 3.74-3.70 (m, 1H), 2.85 (s, 3H), 2.65 (s, 2H), 2.37-2.11 (m, 4H), 1.96-1.69 (m, 2H).

LC-MS (ESI): m/z=435.6 [M+H]⁺

¹H NMR (400 MHz, DMSO-d6) δ 8.90 (s, 2H), 7.96 (s, 1H), 7.37 (s, 1H), 7.29 (s, 1H), 4.92-4.89 (m, 1H), 4.43 (s, 2H), 3.95 (s, 2H), 3.83-3.79 (m, 1H), 3.74-3.70 (m, 1H), 2.85 (s, 3H), 2.65 (s, 2H), 2.34-2.17 (m, 4H), 1.87-1.80 (m, 2H).

### Example 30

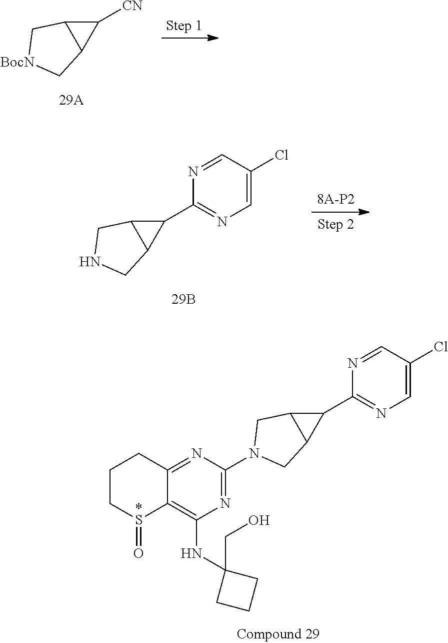

Step 1: In an ice bath, under nitrogen protection, methanol (1 mL, 23.00 mmol) was slowly dropwise added to a system containing 29A (1.50 g, 7.70 mmol, see patent WO 2010078348 A1 for the synthesis) and a 4M hydrogen chloride dioxane solution (6 mL, 23.00 mmol). After the addition was complete, the reaction liquid was stirred at room temperature for 6 h. After the reaction liquid was cooled to 5° C., sodium methoxide (830 mg, 15.4 mmol) and a 7N ammonia methanol solution (1.6 mL) were added and stirred at room temperature for 16 h. At room temperature, a solution of sodium methoxide in methanol (1.10 g dissolved in 4.5 mL of methanol) was added to the reaction liquid and stirred for 30 min. (2-Chloro-3-dimethylamino-prop-2-enylidene)-dimethyl-ammonium hexafluorophosphate (2.00 g) was then added and stirred at room temperature for 3 h. The reaction liquid was concentrated under reduced pressure to a small volume, 2-methyltetrahydrofuran (100 mL) was then added and washed twice with water (80 mL). The organic phases were dried over anhydrous sodium sulfate, and then concentrated in vacuum to obtain a crude product, and the crude product was purified three times by column chromatography (eluents: DCM:MeOH=100:1 to 10:1) to obtain the target compound 29B (120 mg, yield: 8.52%).

LC-MS (ESI): m/z=196.2 [M+H]⁺.

Step 2: Compound 8A-P2 (100 mg, 0.33 mmol) and compound 29B (100 mg, 0.51 mmol) were dissolved in 1,4-dioxane (6 mL), DIPEA (130 mg, 1.00 mmol) was then added, and the mixture was heated to 85° C. and stirred for 16 h under nitrogen protection. After the reaction was complete, the reaction liquid was concentrated under reduced pressure to obtain a crude product, which was purified by column chromatography (eluents: dichloromethane:methanol=100:1 to 10:1) and then further purified by prep.HPLC preparation (preparation method: instrument: waters 2767 preparative liquid chromatographic instrument; chromatographic column: SunFire® Prep C18 (19 mm×250 mm); the sample was dissolved in N,N-dimethylformamide and filtered with a 0.45 μm filter to prepare a sample solution; preparative chromatography conditions: a. composition of mobile phases A and B: mobile phase A: acetonitrile, and mobile phase B: water (containing 5 mM aqueous ammonia); b. gradient elution, mobile phase A with a content of 20-80%; c. flow: 15 mL/min; d. elution time: 15 min; retention time: 7.0 min) to obtain the target compound 29 (3.5 mg, 2.29%).

LCMS m/z=462.1 [M+H]⁺

¹H NMR (400 MHz, CDCl₃) δ 8.61 (s, 2H), 5.86 (s, 1H), 4.88 (s, 1H), 4.11-4.04 (m, 1H), 3.97-3.91 (m, 1H), 3.86 (s, 2H), 3.68-3.59 (m, 3H), 3.45-3.38 (m, 1H), 3.07-2.98 (m, 2H), 2.64-2.49 (m, 2H), 2.37-2.25 (m, 4H), 1.99-1.85 (m, 4H), 1.69-1.62 (m, 1H).

### Example 31

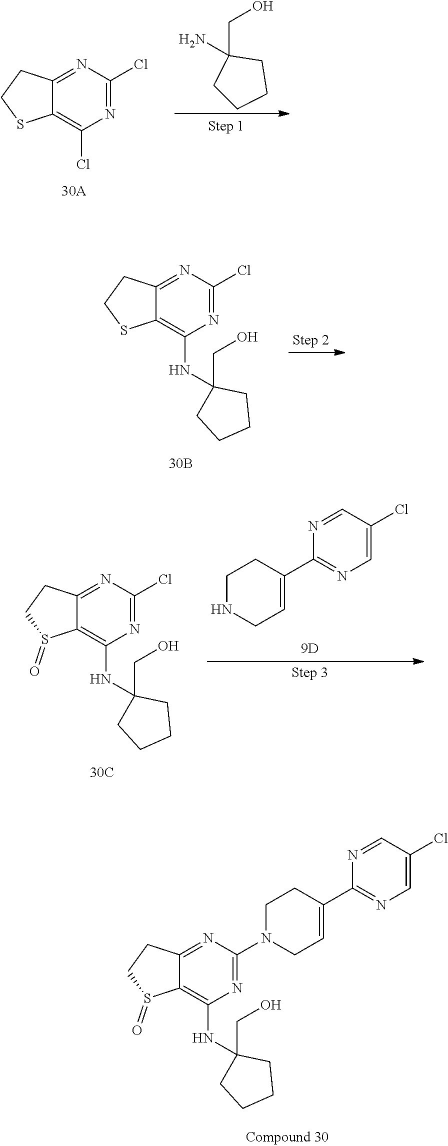 [SMILES: Clc1nc(Cl)c2c(n1)CCS2 ; NC1(CO)CCCC1 ; OCC1(Nc2nc(Cl)nc3c2SCC3)CCCC1 ; O=[S@@]1CCc2nc(Cl)nc(NC3(CO)CCCC3)c21 ; Clc1cnc(C2=CCNCC2)nc1 ; O=[S@@]1CCc2nc(N3CC=C(c4ncc(Cl)cn4)CC3)nc(NC3(CO)CCCC3)c21]

Step 1: Compound 30A (1.0 g, 4.83 mmol) was dissolved in acetonitrile (10 mL), (1-aminocyclopentyl)methanol (610.0 mg, 5.31 mmol) and triethylamine (1.47 g, 14.49 mmol) were then added, and the mixture was heated to 70° C. and reacted for 14 h. The system was then concentrated and subjected to column chromatography (petroleum ether/ethyl acetate=1/1) to obtain compound 30B (500 mg, 36%).

LC-MS (ESI): m/z=286.1 [M+1]⁺

Step 2: Compound 30B (600.0 mg, 1.75 mmol), (S)-(−)-1,1′-bi-2-naphthol (50.0 mg, 0.18 mmol), titanium tetraisopropoxide (25.0 mg, 0.088 mmol), and water (32.0 mg, 1.75 mmol) were dissolved in dichloromethane (10 mL). After nitrogen displacement three times, the system was stirred at room temperature for one hour, and tert-butyl hydroperoxide (170.0 mg, 1.93 mmol) was added and stirred for 1.5 hours. After quenching with a saturated sodium thiosulfate aqueous solution, the aqueous phase was extracted with dichloromethane, dried, concentrated, and subjected to column chromatography (dichloromethane/methanol=10/1) to obtain compound 30C (430.0 mg, 81%).

LC-MS (ESI): m/z=302.1 [M+H]⁺.

Step 3: Compound 30C (430.0 mg, 1.42 mmol) was dissolved in 1,4-dioxane (10.0 mL), compound 9D (280.0 mg, 1.42 mmol) and triethylamine (550 mg, 4.26 mmol) were then added, and the mixture was heated to 90° C. and reacted for 12 h. The system was then cooled to room temperature, concentrated, and subjected to column chromatography (dichloromethane/methanol=10/1) to obtain compound 30 (290.0 mg, 44%).

LC-MS (ESI): m/z=461.1 [M+H]⁺.

¹H NMR (400 MHz, DMSO-d₆) δ 8.90 (s, 2H), 7.35-7.21 (m, 1H), 6.58 (s, 1H), 4.91 (t, 1H), 4.48 (s, 2H), 3.99 (t, 2H), 3.74-3.60 (m, 2H), 3.48-3.36 (m, 1H), 3.32-3.22 (m, 1H), 3.00-2.91 (m, 1H), 2.90-2.82 (m, 1H), 2.66 (s, 2H), 2.20-2.05 (m, 2H), 1.88-1.78 (m, 2H), 1.74-1.63 (m, 2H), 1.62-1.52 (m, 2H).

### Example 32

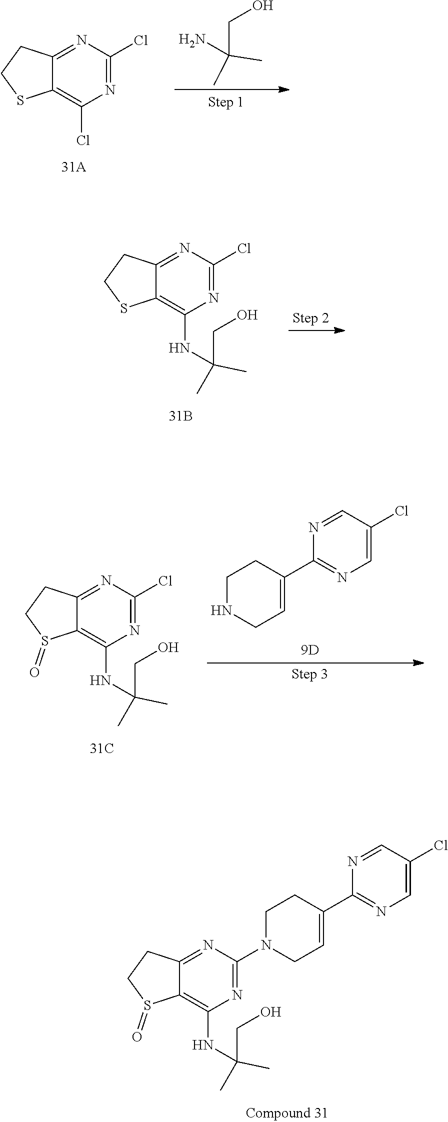

Step 1: Compound 31A (2.0 g, 9.64 mmol) was dissolved in acetonitrile (20 mL), 2-amino-2-methylpropan-1-ol (950.0 mg, 10.64 mmol) and triethylamine (2.93 g, 28.92 mmol) were then added, and the mixture was heated to 70° C. and reacted for 14 h. The system was then concentrated and subjected to column chromatography (petroleum ether/ethyl acetate=1/1) to obtain compound 31B (600 mg, 24%).

LC-MS (ESI): m/z=260.1 [M+1]⁺. Step 2: Compound 31B (600.0 mg, 2.31 mmol) was dissolved in dichloromethane (10 mL), and m-chloroperoxybenzoic acid (600.0 mg, 3.49 mmol) was added and stirred for 16 hours. After quenching with a saturated sodium thiosulfate aqueous solution, the aqueous phase was extracted with dichloromethane, dried, concentrated, and subjected to column chromatography (dichloromethane/methanol=10/1) to obtain compound 31C (632.0 mg, 99%).

LC-MS (ESI): m/z=276.1 [M+H]⁺.

Step 3: Compound 31C (632.0 mg, 2.29 mmol) was dissolved in 1,4-dioxane (10.0 mL), compound 9D (540.0 mg, 2.75 mmol) and N,N-diisopropylethylamine (890 mg, 6.87 mmol) were then added, and the mixture was heated to 90° C. and reacted for 12 h. The system was then cooled to room temperature, concentrated, and subjected to column chromatography (dichloromethane/methanol=10/1) to obtain compound 31 (440.0 mg, 44%).

LC-MS (ESI): m/z=435.1 [M+H]⁺

¹H NMR (400 MHz, DMSO-d₆) δ 8.90 (s, 2H), 7.38-7.16 (m, 1H), 6.23 (s, 1H), 5.15 (t, 1H), 4.49 (s, 2H), 4.00 (t, 2H), 3.51 (d, 2H), 3.48-3.33 (m, 2H), 3.01-2.91 (m, 1H), 2.91-2.82 (m, 1H), 2.67 (s, 2H), 1.43 (s, 6H).

### Example 33

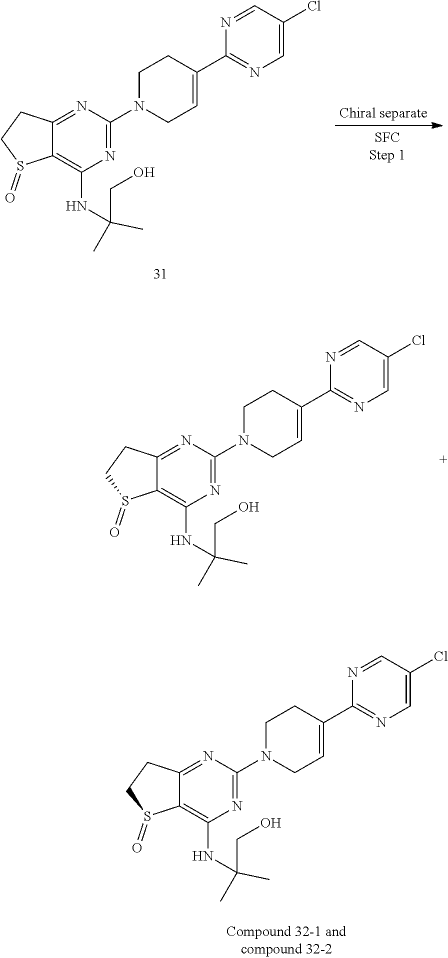 [SMILES: CC(C)(CO)Nc1nc(N2CC=C(c3ncc(Cl)cn3)CC2)nc2c1S(=O)CC2 ; CC(C)(CO)Nc1nc(N2CC=C(c3ncc(Cl)cn3)CC2)nc2c1[S@](=O)CC2 ; CC(C)(CO)Nc1nc(N2CC=C(c3ncc(Cl)cn3)CC2)nc2c1[S@@](=O)CC2]

Step 1: Compound 31 (380 mg) was subjected to chiral resolution to obtain compound 32-1 (165 mg) and compound 32-2 (180 mg).

Preparation method: instrument: Waters 150 SFC, column: Chiralpak AS, mobile phase: (A for CO₂ and B for MeOH (0.1% NH₃·H₂O); gradient: 40% phase B elution; flow rate: 120 mL/min, back pressure: 100 bar, column temperature: 25° C.; wavelength: 220 nm; cycle time: 2.8 min; sample preparation: sample concentration 5 mg/mL, acetonitrile solution injection: 1.0 mL each time. After separation, the fraction was dried on a rotary evaporator at a bath temperature of 35° C. to obtain P1 (retention time: 1.730 minutes, assigned as compound 32-1) and P2 (retention time: 1.948 minutes, assigned as compound 32-2).

LC-MS (ESI): m/z=435.1 [M+H]⁺

¹H NMR (400 MHz, DMSO-d₆) δ 8.89 (s, 2H), 7.34-7.26 (m, 1H), 6.21 (s, 1H), 5.13 (t, 1H), 4.49 (d, 2H), 4.00 (t, 2H), 3.51 (d, 2H), 3.46-3.33 (m, 2H), 3.01-2.81 (m, 2H), 2.68 (d, 2H), 1.43 (s, 6H).

LC-MS (ESI): m/z=435.1 [M+H]⁺

¹H NMR (400 MHz, DMSO-d₆) δ 8.89 (s, 2H), 7.34-7.26 (m, 1H), 6.21 (s, 1H), 5.13 (t, 1H), 4.49 (d, 2H), 4.00 (t, 2H), 3.51 (d, 2H), 3.47-3.32 (m, 2H), 3.02-2.81 (m, 2H), 2.67 (d, 2H), 1.43 (s, 6H).

### Example 34

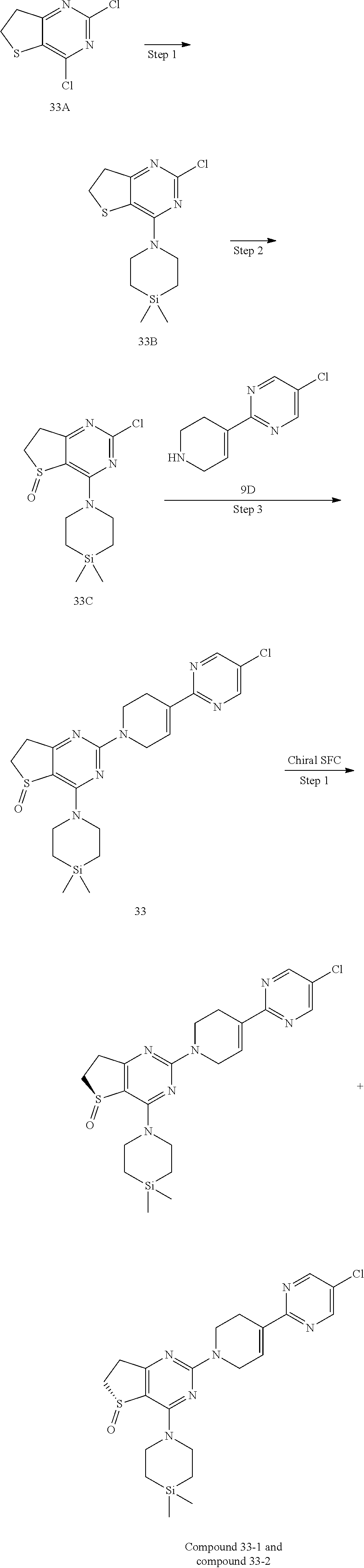

Using compound 33A and 4,4-dimethyl-[1,4]silapiperidine hydrochloride as raw materials, compound 33 (0.6 g, 45%) was obtained according to the operation of Example 31.

LC-MS (ESI): m/z=474.1 [M+H]⁺.

Compound 33 (600 mg) was subjected to chiral resolution to obtain compound 33-1 (200 mg) and compound 33-2 (202 mg).

Preparation method: instrument: Waters 150 SFC, column: Chiralcel column (250×30 mm×10 um), mobile phase: (A for CO₂; B for 0.1% NH₃·H₂O in MEOH); gradient: 35% phase B elution; flow rate: 100 mL/min, back pressure: 100 bar, column temperature: 25° C.; wavelength: 220 nm; cycle time: 3.0 min; sample preparation: sample concentration 10 mg/mL, acetonitrile solution injection: 3.0 mL each time. After separation, the fraction was dried on a rotary evaporator at a bath temperature of 35° C. to obtain P1 (retention time: 1.592 minutes, assigned as compound 33-1) and P2 (retention time: 2.079 minutes, assigned as compound 33-2).

LC-MS (ESI): m/z=474.1 [M+H]⁺.

¹H NMR (400 MHz, DMSO-d6) δ 8.78 (s, 2H), 7.18 (s, 1H), 4.37 (s, 2H), 4.05 (d, 2H), 3.90-3.87 (m, 4H), 3.41-3.36 (m, 1H), 3.13-3.10 (m, 1H), 2.98-2.79 (m, 2H), 2.56 (s, 2H), 0.90-0.64 (m, 4H), 0.00 (s, 6H).

LC-MS (ESI): m/z=474.1 [M+H]⁺.

¹H NMR (400 MHz, DMSO-d6) δ 8.78 (s, 2H), 7.19 (s, 1H), 4.37 (s, 2H), 4.05 (d, 2H), 3.90-3.87 (m, 4H), 3.46-3.31 (m, 1H), 3.16-3.08 (m, 1H), 2.97-2.78 (m, 2H), 2.56 (s, 2H), 0.88-0.63 (m, 4H), 0.00 (s, 6H).

### Example 35

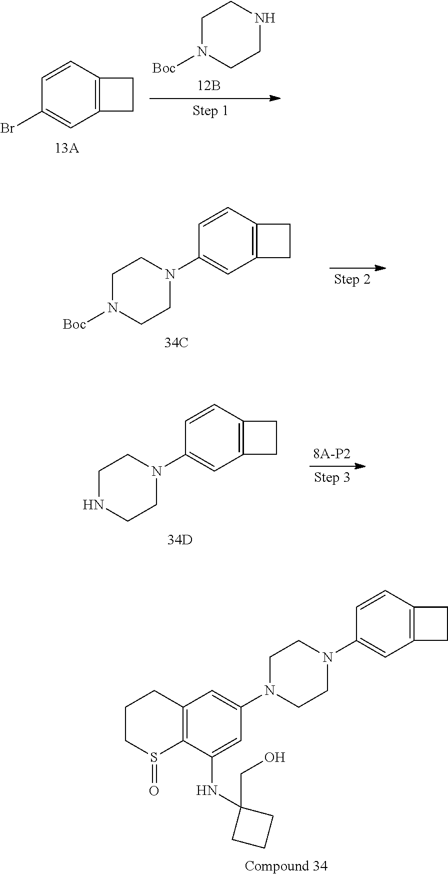

Using compounds 13A and 12B as raw materials, compound 34 (160 mg, 53%) was obtained according to the operation of Example 14.

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A with a content of 40-80%; c. flow: 15 mL/min; d. elution time: 20 min; retention time: 12.60 min.

LCMS m/z=454.2 [M+1]⁺

¹H NMR (400 MHZ, DMSO-d6) δ 6.92 (d, 1H), 6.78-6.81 (m, 1H), 6.75 (s, 1H), 6.53 (s, 1H), 4.88 (t, 1H), 3.82 (t, 4H), 3.71 (d, 2H), 3.01-3.09 (m, 9H), 2.88-2.94 (m, 1H), 2.52-2.70 (m, 2H), 2.29-2.43 (m, 3H), 2.09-2.19 (m, 2H), 1.90-2.00 (m, 1H), 1.72-1.86 (m, 2H).

### Example 36

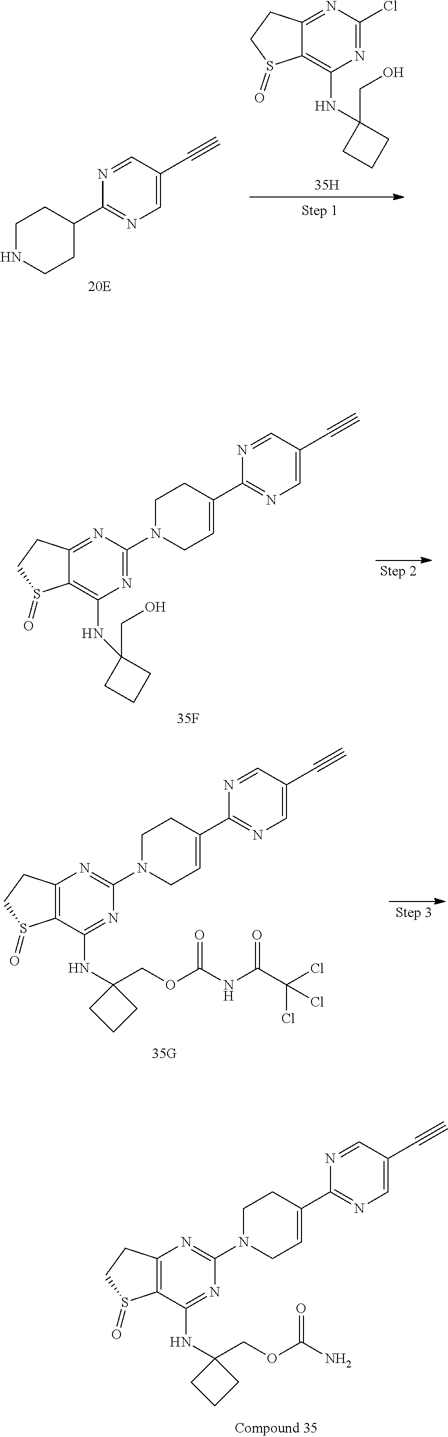 [SMILES: C#Cc1cnc(C2CCNCC2)nc1 ; O=S1CCc2nc(Cl)nc(NC3(CO)CCC3)c21 ; C#Cc1cnc(C2=CCN(c3nc4c(c(NC5(CO)CCC5)n3)[S@](=O)CC4)CC2)nc1 ; C#Cc1cnc(C2=CCN(c3nc4c(c(NC5(COC(=O)NC(=O)C(Cl)(Cl)Cl)CCC5)n3)[S@](=O)CC4)CC2)nc1 ; C#Cc1cnc(C2=CCN(c3nc4c(c(NC5(COC(N)=O)CCC5)n3)[S@](=O)CC4)CC2)nc1]

Step 1: Compound 20E (210.0 mg, 1.14 mmol) was dissolved in 1,4-dioxane (20.0 mL), compound 35H (345.0 mg, 1.2 mmol, see patent WO 2014124860 A1 for the synthesis) was then added, and the mixture was heated back to 90° C. and reacted for 12 h. The system was then cooled to room temperature, concentrated, and subjected to column chromatography (dichloromethane/methanol=10/1) to obtain compound 35F (360.0 mg, 72%).

LC-MS (ESI): m/z=437.1 [M+H]⁺.

¹H NMR (400 MHz, DMSO-d6) δ 8.90 (s, 2H), 7.36 (s, 1H), 7.34 (s, 1H), 4.84 (t, 1H), 4.68 (s, 1H), 4.48 (s, 2H), 3.97 (t, 2H), 3.75 (d, 2H), 3.51-3.36 (m, 1H), 3.28-3.18 (m, 1H), 2.98-2.84 (m, 2H), 2.66 (s, 2H), 2.43-2.30 (m, 2H), 2.24-2.17 (m, 2H), 1.86-1.71 (m, 2H).

Step 2: Compound 35F (0.8 g, 2.0 mmol) was dissolved in dichloromethane (15 mL), trichloroacetyl isocyanate (452.0 mg, 2.4 mmol) was added in an ice bath, and the mixture was further reacted for 1 h. The system was concentrated to obtain 35G (1.2 g, 99%), which was directly used for the next step.

LC-MS (ESI): m/z=624.0 [M+1]⁺

Step 3: Compound 35G (1.2 g, 2.0 mmol) was dissolved in methanol (15 mL), water (15 mL) and potassium carbonate (1.0 g, 8.0 mmol) were then added, and the mixture reacted at room temperature for 2.5 hours. After concentration, the system was extracted by adding dichloromethane, dried, and concentrated to obtain compound 35 (0.6 g, 62%).

LC-MS (ESI): m/z=480.2 [M+1]⁺

¹H NMR (400 MHz, DMSO-d6) δ 8.90 (s, 2H), 7.61 (s, 1H), 7.34 (s, 1H), 6.48 (s, 2H), 4.68 (s, 1H), 4.48 (s, 2H), 4.44-4.32 (m, 2H), 3.99-3.96 (m, 2H), 3.52-3.36 (m, 1H), 3.27-3.11 (m, 1H), 2.99-2.85 (m, 2H), 2.66 (s, 2H), 2.41-2.24 (m, 4H), 1.95-1.74 (m, 2H).

### Example 37

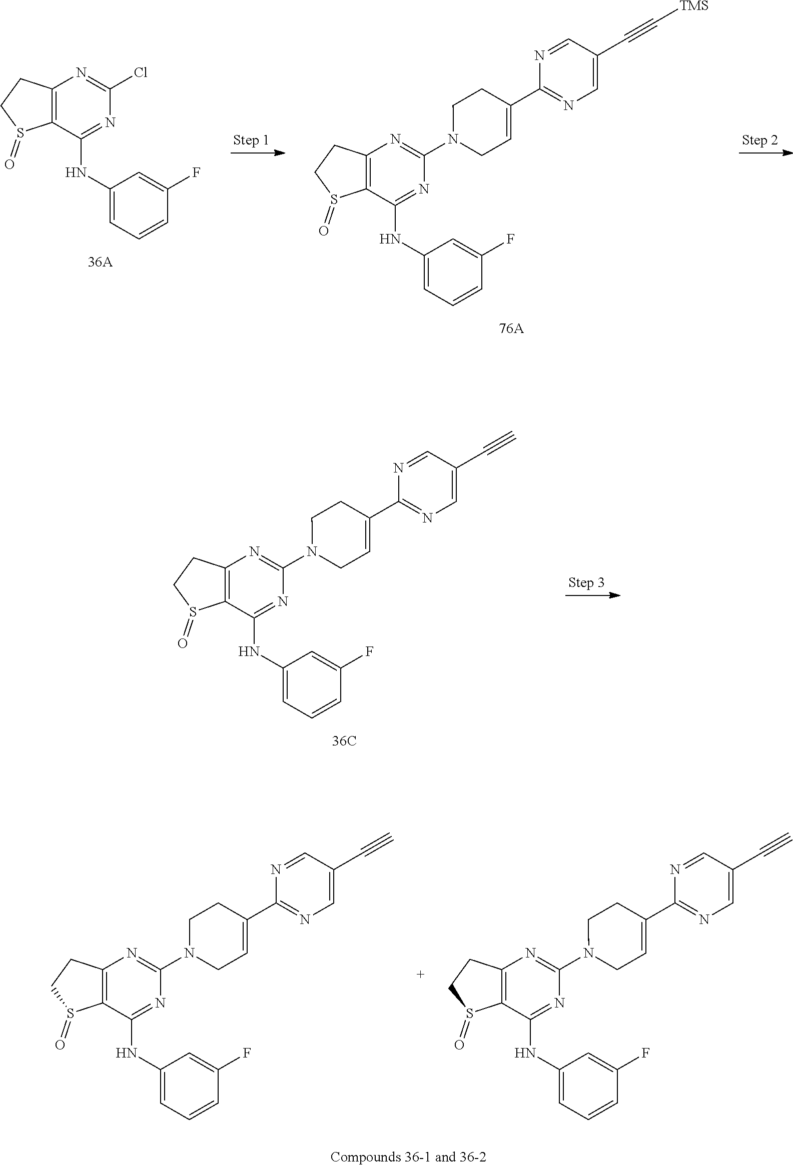 [SMILES: O=S1CCc2nc(Cl)nc(Nc3cccc(F)c3)c21 ; C[Si](C)(C)C#Cc1cnc(C2=CCN(c3nc4c(c(Nc5cccc(F)c5)n3)S(=O)CC4)CC2)nc1 ; C#Cc1cnc(C2=CCN(c3nc4c(c(Nc5cccc(F)c5)n3)S(=O)CC4)CC2)nc1 ; C#Cc1cnc(C2=CCN(c3nc4c(c(Nc5cccc(F)c5)n3)[S@](=O)CC4)CC2)nc1 ; C#Cc1cnc(C2=CCN(c3nc4c(c(Nc5cccc(F)c5)n3)[S@@](=O)CC4)CC2)nc1]

Step 1: 36A (360 mg, 1.21 mmol, see patent WO 2011124524 A1 for the synthesis) was dissolved in 1,4-dioxane (8 mL), 20D (515 mg, 1.45 mmol) and N,N-diisopropylethylamine (780 mg, 6.05 mmol) were successively added, and the mixture was heated to 85° C. and stirred for 16 h under nitrogen protection. After the reaction liquid naturally returned to room temperature, the reaction was diluted by adding water (50 mL) and extracted with ethyl acetate (50 mL) three times. The organic phases were combined, dried over anhydrous sodium sulfate, and then concentrated in vacuum to obtain a crude product, which was purified by column chromatography (eluents: DCM:MeOH=100:1 to 15:1) to obtain the target compound 36B (590 mg, yield: 94.07%).

LC-MS (ESI): m/z=519.2 [M+H]⁺.

Step 2: Compound 36B (550 mg, 1.06 mmol) was dissolved in methanol (15 mL), potassium carbonate (150 mg, 1.06 mmol) was added, and the mixture was stirred at room temperature for 2 h. After the raw material disappeared as detected by a TLC plotting plate (developing agent: DCM:MeOH=15:1), the mixture was diluted by adding water (50 mL) and extracted three times with dichloromethane (50 mL). The organic phases were combined, dried over anhydrous sodium sulfate, and then concentrated in vacuum to obtain a crude product, which was purified by column chromatography (eluents: DCM:MeOH=100:1 to 10:1) to obtain the target compound 36C (350 mg, yield: 73.92%).

LC-MS (ESI): m/z=447.2 [M+H]⁺.

Step 3: Compound 36C (350 mg, 0.78 mmol) was subjected to chiral resolution to obtain compound 36-1 (13.0 mg) and compound 36-2 (3.2 mg).

Analysis method: instrument: SHIMADZU LC-20AD, column: Chiralpak IG-3 50×4.6 mm I.D., 3 μm; mobile phase: A: heptane (0.05%, N,N-diethylaniline), B: ethanol (0.05% N,N-diethylaniline); gradient: 20% B in A; flow rate: 1 mL/min, column temperature: 35° C., wavelength: 254 nm.

Preparation method: instrument: SHIMADZU LC-20AP, column: DAICEL CHIRALPAK IG (250 mm*30 mm, 10 μm), mobile phase: A n-hexane, B ethanol (0.1% NH₃·H2O); gradient: 8% B gradient elution, flow rate: 110 mL/min, column temperature: 25° C., wavelength: 254 nm, cycle time: 16 min, sample preparation: sample concentration 1.5 mg/ml, ethanol solution injection: 2 ml each time. After separation, the fraction was dried by a rotary evaporator at a bath temperature of 40° C. to obtain compound 36-1 (retention time: 5.716 minutes) and compound 36-2 (retention time: 6.890 minutes).

Compound 36-1: LCMS m/z=447.2 [M+H]⁺

¹H NMR (400 MHz, CDCl₃) δ 8.77 (s, 2H), 8.09 (s, 1H), 7.64-7.61 (m, 1H), 7.37 (s, 1H), 7.23-7.18 (m, 2H), 6.76-6.72 (m, 1H), 4.56-4.47 (m, 2H), 4.10-4.00 (m, 2H), 3.67-3.59 (m, 1H), 3.45-3.39 (m, 2H), 3.17-3.03 (m, 2H), 2.86-2.77 (m, 2H).

Compound 36-2: LCMS m/z=447.2 [M+H]⁺

¹H NMR (400 MHz, CDCl₃) δ 8.77 (s, 2H), 8.09 (s, 1H), 7.64-7.61 (m, 1H), 7.37 (s, 1H), 7.23-7.17 (m, 2H), 6.76-6.72 (m, 1H), 4.54-4.49 (m, 2H), 4.10-4.01 (m, 2H), 3.67-3.59 (m, 1H), 3.45-3.39 (m, 2H), 3.17-3.03 (m, 2H), 2.86-2.77 (m, 2H).

### Example 38

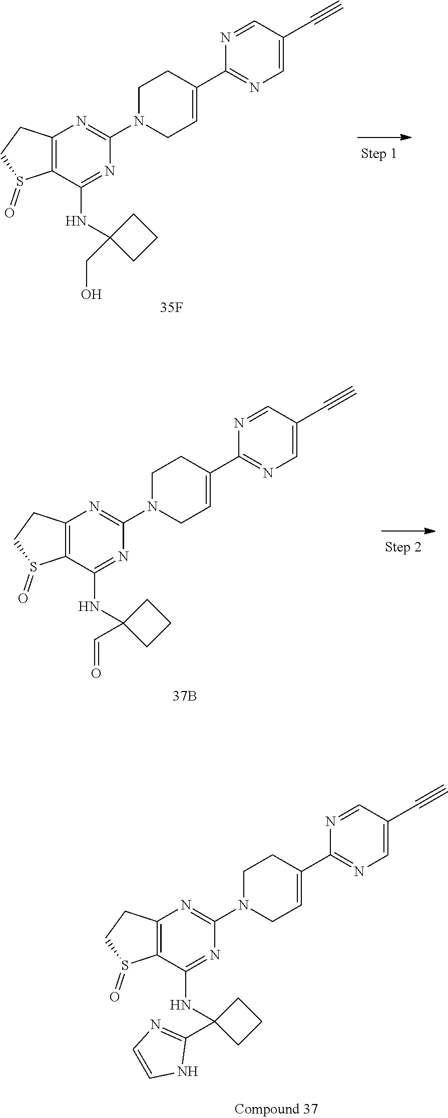 [SMILES: C#Cc1cnc(C2=CCN(c3nc4c(c(NC5(CO)CCC5)n3)[S@](=O)CC4)CC2)nc1 ; C#Cc1cnc(C2=CCN(c3nc4c(c(NC5(C=O)CCC5)n3)[S@](=O)CC4)CC2)nc1 ; C#Cc1cnc(C2=CCN(c3nc4c(c(NC5(c6ncc[nH]6)CCC5)n3)[S@](=O)CC4)CC2)nc1]

Step 1: Compound 35F (2.0 g, 4.58 mmol) was dissolved in dichloromethane (100 mL), Dess-Martin periodinane (2.92 g, 6.88 mmol) was added, and the mixture was stirred at room temperature overnight. The reaction liquid was quenched with a saturated sodium thiosulfate aqueous solution, and the aqueous phase was extracted with dichloromethane. The organic phase was dried, filtered, concentrated, and subjected to column chromatography (dichloromethane/methanol=10/1) to obtain compound 37B (1.2 g, 60%).

LC-MS (ESI): m/z=435.2 [M+H]⁺

Step 2: Compound 37B (220 mg, 0.51 mmol) was dissolved in an ammonia methanol solution (5 mL, 7.0 M) and stirred at room temperature for half an hour, and glyoxal (1 mL, 40 wt. % in water) was then added and stirred at room temperature overnight. The reaction liquid was concentrated and subjected to silica gel column chromatography (dichloromethane:methanol=10:1) to obtain 130 mg of a pale yellow solid, and after reverse phase preparation (acetonitrile:water=1/99-99/1), compound 37 (100 mg, 40%) was obtained.

LC-MS (ESI): m/z=473.2 [M+H]⁺.

¹H NMR (400 MHz, DMSO-d₆) δ 11.38 (s, 1H), 8.89 (s, 2H), 8.20 (s, 1H), 7.24 (s, 1H), 6.90 (s, 1H), 6.79 (s, 1H), 4.68 (s, 1H), 4.60-3.95 (m, 2H), 3.94-3.58 (m, 2H), 3.49-3.37 (m, 1H), 3.25-3.15 (m, 1H), 3.01-2.76 (m, 3H), 2.65-2.51 (m, 5H), 2.02-1.88 (m, 2H).

### Example 39

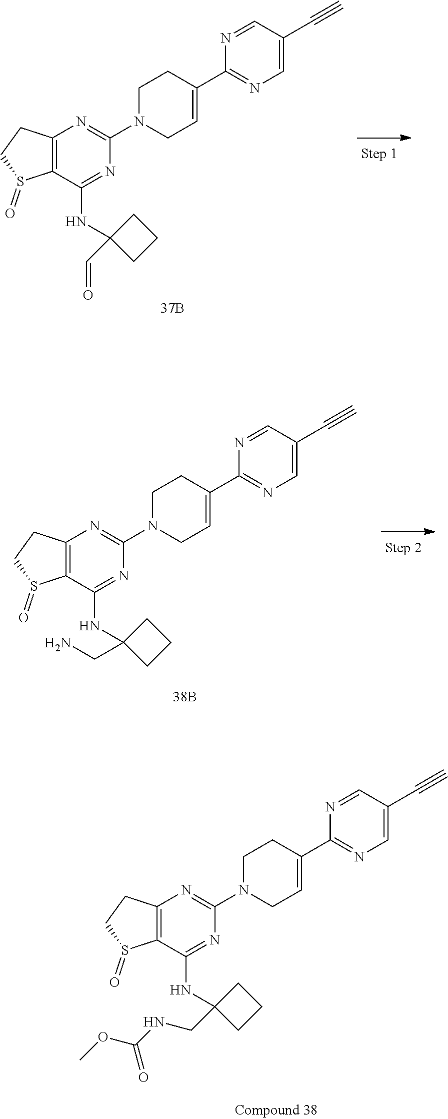

Step 1: Compound 37B (1.2 g, 2.76 mmol) was dissolved in 1,2-dichloroethane (100 mL), ammonium acetate (21.3 g, 275.94 mmol) was added and stirred at room temperature overnight, and sodium triacetoxyborohydride (5.85 g, 27.60 mmol) was then added. The mixture was reacted at room temperature for 2 h. The reaction was quenched by adding a saturated sodium bicarbonate solution. The aqueous phase was extracted with dichloromethane, and the organic phases were combined, dried, filtered, concentrated, and subjected to silica gel column chromatography (dichloromethane:methanol:aqueous ammonia=10:1:0.1) to obtain compound 38B (180 mg, 15%).

LC-MS (ESI): m/z=436.2 [M+H]⁺.

Step 2: Compound 38B (100 mg, 0.23 mmol) was dissolved in dichloromethane (10 mL), triethylamine (35 mg, 0.35 mmol) was added in an ice bath and stirred for five minutes, and methyl chloroformate (24 mg, 0.25 mmol) was then added. The mixture was reacted at 0° C. for 1 h. The reaction was quenched by adding water, the aqueous phase was extracted with dichloromethane, and the organic phases were combined, dried, filtered, concentrated, and subjected to reverse phase preparation (acetonitrile:water=1/99-99/1) to obtain compound 38 (50 mg, 44%).

LC-MS (ESI): m/z=494.2 [M+H]⁺

¹H NMR (400 MHz, DMSO-d₆) δ 8.90 (s, 2H), 7.49 (s, 1H), 7.40-7.31 (m, 1H), 7.18 (t, 1H), 4.68 (s, 1H), 4.49 (s, 2H), 3.99 (t, 2H), 3.57-3.38 (m, 6H), 3.27-3.16 (m, 1H), 3.01-2.82 (m, 2H), 2.67 (s, 2H), 2.42-2.18 (m, 4H), 1.88-1.68 (m, 2H).

### Example 40

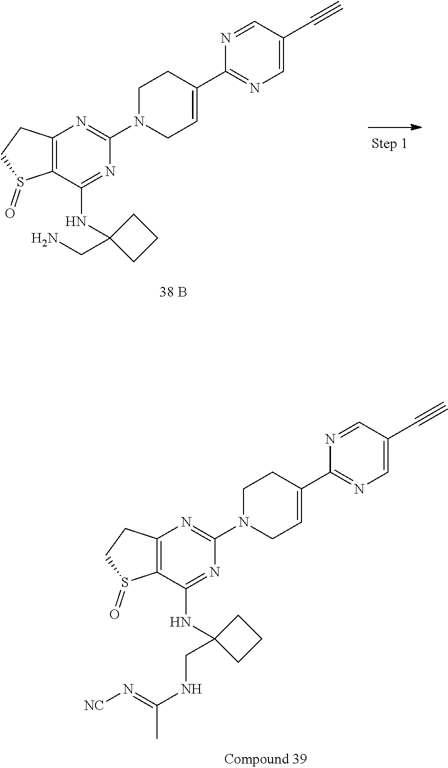 [SMILES: C#Cc1cnc(C2=CCN(c3nc4c(c(NC5(CN)CCC5)n3)[S@](=O)CC4)CC2)nc1 ; C#Cc1cnc(C2=CCN(c3nc4c(c(NC5(CN/C(C)=N/C#N)CCC5)n3)[S@](=O)CC4)CC2)nc1]

Step 1: Compound 38B (100 mg, 0.23 mmol) was dissolved in methanol (5 mL), methyl N-cyanoethanimideate (25 mg, 0.26 mmol) was added, and the mixture was stirred at room temperature overnight. The reaction liquid was concentrated and subjected to reverse phase preparation (acetonitrile:water=1/99-99/1) to obtain compound 39 (50 mg, 43%).

LC-MS (ESI): m/z=502.2 [M+H]⁺

¹H NMR (400 MHz, DMSO-d₆) δ 8.90 (s, 2H), 8.72 (s, 1H), 7.53 (s, 1H), 7.35 (s, 1H), 4.69 (s, 1H), 4.49 (s, 2H), 3.98 (t, 2H), 3.89-3.81 (m, 1H), 3.78-3.68 (m, 1H), 3.50-3.39 (m, 1H), 3.25-3.18 (m, 1H), 3.03-2.79 (m, 2H), 2.72-2.64 (m, 2H), 2.41-2.23 (m, 4H), 2.21 (s, 3H), 1.86-1.74 (m, 2H).

### Example 41

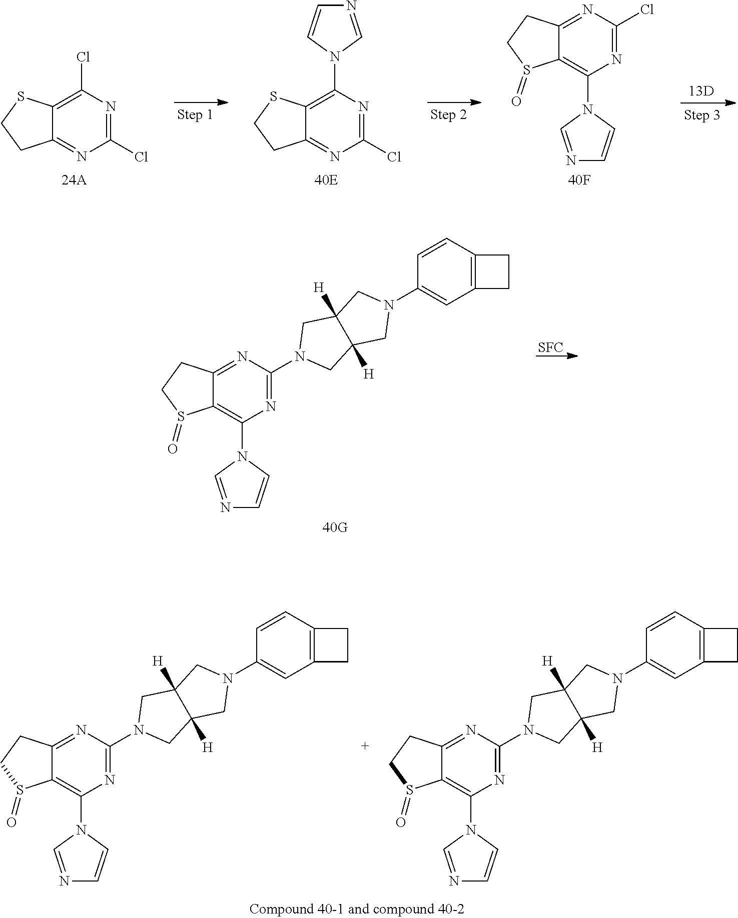

Step 1: Compound 24A (3.00 g, 14.46 mmol) and imidazole (1.18 g, 17.35 mmol) were weighed into a 100 mL single-mouth flask and dissolved with acetonitrile (30 mL), and N,N-diisopropylethylamine (5.61 g, 43.38 mmol) was added. After the addition was complete, the system was stirred at 70 degrees Celsius for 16 h. After the reaction was complete as monitored by a TLC spotting plate (petroleum ether:ethyl acetate=2:1), water (50 mL) was added and stirred for 5 min, and the mixture was extracted with ethyl acetate (30 mL). The organic phase was separated, dried over anhydrous sodium sulfate, and concentrated under reduced pressure, and the crude product was purified by column chromatography (eluents: petroleum ether:ethyl acetate=2:1) to obtain the target compound 40E (1.0 g, yield: 29%).

LCMS m/z=239.1 [M+1]⁺

Step 2: Compound 40E (1.0 g, 4.19 mmol) was weighed into a 100 mL single-mouth flask and dissolved with dichloromethane (10 mL), and tert-butyl hydroperoxide (0.65 g, 5.03 mmol) was added and stirred at room temperature for 12 h. After the reaction was complete as monitored by a TLC spotting plate (petroleum ether:ethyl acetate=2:1), water (50 mL) was added and stirred for 5 min, and the mixture was extracted with dichloromethane (30 mL). The organic phase was separated, dried over anhydrous sodium sulfate, and concentrated under reduced pressure, and the crude product was purified by column chromatography (eluents: dichloromethane:methanol=20:1) to obtain the target compound 40F (0.63 g, yield: 59%).

LCMS m/z=255.1 [M+1]⁺

Step 3: Compound 40F (300 mg, 1.18 mmol) was added to a 100 mL single-mouth flask and dissolved with dioxane (10 mL), and compound 40C (300 mg, 1.42 mmol) and N,N-diisopropylethylamine (0.46 g, 3.54 mmol) were then added. After the addition was complete, the system was placed under nitrogen protection and stirred at 85° C. for 16 h. After the reaction was complete as monitored by TLC (dichloromethane:methanol=10:1), water (50 mL) was added and stirred for 5 min, and the mixture was extracted with dichloromethane (30 mL). The organic phase was separated, dried over anhydrous sodium sulfate, and concentrated under reduced pressure, and the crude product was purified by column chromatography (eluents:dichloromethane:methanol=10:1) to obtain the target compound 40G, which was subjected to chiral resolution to obtain the target compound 40-1 (200 mg) and compound 40-2 (120 mg).

Chiral resolution method: instrument: Waters 150 SFC; column: Chiralpak IC Column (250×30 mm, I.D 30 mm, 10 μm particle size); mobile phase: A for CO₂ and B for IPA+ACN (0.1% NH₃·H₂O); gradient: 45% phase B isocratic elution; flow rate: 100 mL/min; back pressure: 100 bar; column temperature: 25° C.; wavelength: 220 nm.

LCMS m/z=433.2 [M+1]⁺

Compound 40-1, ¹H NMR (400 MHZ, DMSO-d6) δ 8.59 (s, 1H), 8.00 (s, 1H), 7.23 (s, 1H), 6.85 (d, 1H), 6.34-6.37 (m, 2H), 3.88-3.98 (m, 2H), 3.58-3.77 (m, 3H), 3.40-3.48 (m, 3H), 3.12-3.37 (m, 6H), 2.98-3.05 (m, 4H).

Compound 40-2, ¹H NMR (400 MHZ, DMSO-d6) δ 8.59 (s, 1H), 8.00 (s, 1H), 7.23 (s, 1H), 6.85 (d, 1H), 6.34-6.37 (m, 2H), 3.88-3.99 (m, 2H), 3.58-3.77 (m, 3H), 3.40-3.48 (m, 3H), 3.12-3.37 (m, 6H), 2.97-3.05 (m, 4H).

### Example 42

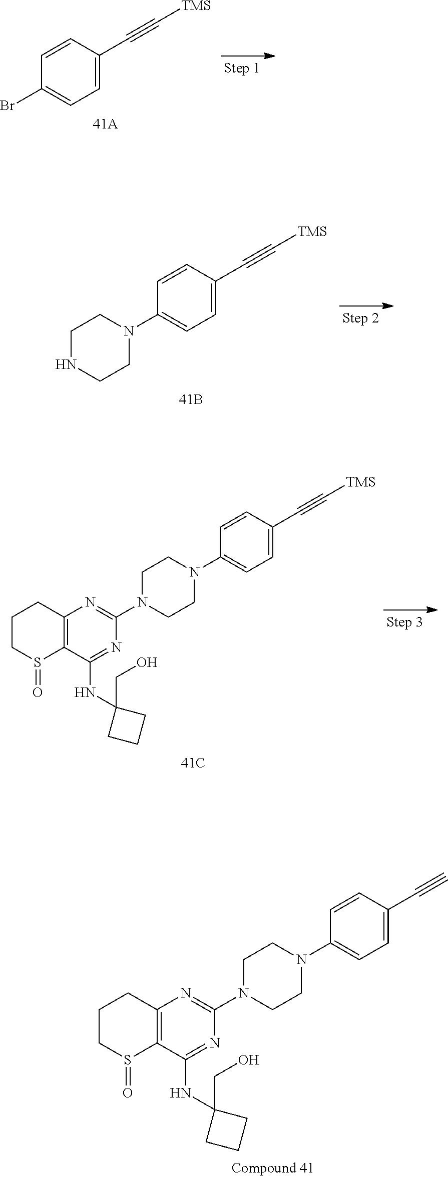 [SMILES: C[Si](C)(C)C#Cc1ccc(Br)cc1 ; C[Si](C)(C)C#Cc1ccc(N2CCNCC2)cc1 ; C[Si](C)(C)C#Cc1ccc(N2CCN(c3nc4c(c(NC5(CO)CCC5)n3)S(=O)CCC4)CC2)cc1 ; C#Cc1ccc(N2CCN(c3nc4c(c(NC5(CO)CCC5)n3)S(=O)CCC4)CC2)cc1]

Step 1: Compound 41A (1.0 g, 3.95 mmol) and piperazine (0.41 g, 4.7 mmol) were weighed into a 100 mL flask and then dissolved by adding dioxane (20 mL), and sodium tert-butoxide (0.76 g, 7.9 mmol), tris(dibenzylideneacetone)dipalladium (0.11 g, 0.20 mmol), and 4,5-bis(diphenylphosphino)-9,9-dimethylxanthene (250 mg, 0.40 mmol) were successively added. After the addition was complete, the mixture was stirred at 80° C. for 12 h. After the reaction was complete as monitored by TLC (petroleum ether:ethyl acetate=5:1), water (20 mL) was added and stirred for 5 min, and the mixture was extracted by adding ethyl acetate (20 mL). The organic phase was separated, the organic phase was dried over anhydrous sodium sulfate and concentrated under reduced pressure, and the crude product was purified by column chromatography (eluents: petroleum ether:ethyl acetate=5:1) to obtain the target compound 41B (0.50 g, 47%).

LCMS m/z=259.1 [M+1]⁺

Step 2: Compound 8A-P2 (350 mg, 1.16 mmol) and compound 41B (300 mg, 1.16 mmol) were added to a 100 mL single-mouth flask and dissolved with dioxane (10 mL), and N,N-diisopropylethylamine (0.45 g, 3.48 mmol) was finally added. After the addition was complete, the mixture was stirred at 90° C. for 16 h under nitrogen protection. After the reaction was complete as monitored by TLC (dichloromethane:methanol=10:1), water (20 mL) was added and stirred for 5 min, and the mixture was extracted by adding ethyl acetate (20 mL). The organic phase was separated, the organic phase was dried over anhydrous sodium sulfate and concentrated under reduced pressure, and the crude product was purified by column chromatography (eluents:dichloromethane:methanol=10:1) to obtain the target compound 41C (0.40 g, 66%).

LCMS m/z=524.2 [M+1]⁺

Step 3: Compound 41C (0.35 g, 0.67 mmol) was weighed into a 100 mL single-mouth flask and dissolved with methanol (10 mL), and potassium carbonate (0.27 g, 1.94 mmol) was finally added. After the addition was complete, the mixture was stirred for 2 h. After the reaction was complete as monitored by TLC (dichloromethane:methanol=10:1), the reaction liquid was filtered under reduced pressure and then concentrated to obtain the target compound. The crude product was purified by preparative HPLC to obtain the title compound 41 (200 mg, 66%).

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A with a content of 40-80%; c. flow: 15 mL/min; d. elution time: 20 min; retention time: 12.50 min.

LCMS m/z=452.2 [M+1]⁺

¹H NMR (400 MHZ, DMSO-d6) δ 7.31 (d, 2H), 6.94 (d, 2H), 6.55 (s, 1H), 4.89 (s, 1H), 3.92 (s, 1H), 3.83 (t, 4H), 3.71 (s, 2H), 3.25 (t, 4H), 3.05-3.09 (m, 1H), 2.88-2.95 (m, 1H), 2.53-2.71 (m, 2H), 2.28-2.45 (m, 3H), 2.09-2.21 (m, 2H), 1.91-2.00 (m, 1H), 1.73-1.88 (m, 2H).

### Example 43

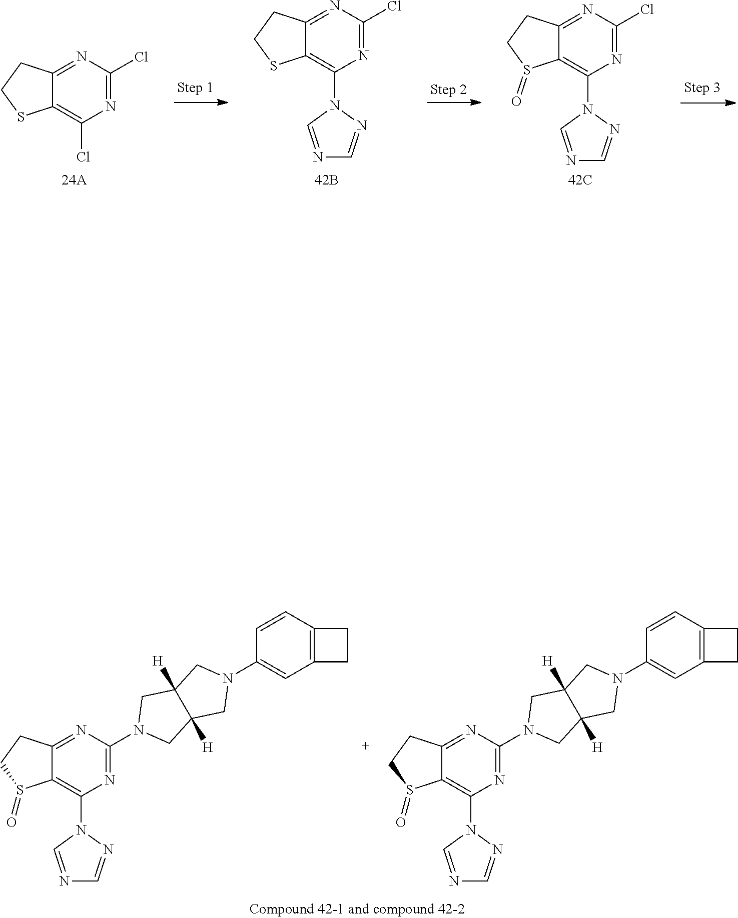 [SMILES: Clc1nc(Cl)c2c(n1)CCS2 ; Clc1nc2c(c(-n3cncn3)n1)SCC2 ; O=S1CCc2nc(Cl)nc(-n3cncn3)c21 ; O=[S@@]1CCc2nc(N3C[C@H]4CN(c5ccc6c(c5)CC6)C[C@H]4C3)nc(-n3cncn3)c21 ; O=[S@]1CCc2nc(N3C[C@H]4CN(c5ccc6c(c5)CC6)C[C@H]4C3)nc(-n3cncn3)c21]

Using compound 24A and 1,2,4-triazole as raw materials, compounds 42-1 and 42-2 were obtained according to the operation of Example 41.

Chiral resolution method: instrument: Waters 150 Prep-SFC F; SFC column: Chiralcel OJ-Column; mobile phase: A for CO₂; B for 0.1% NH₃·H₂O in IPA and ACN; gradient: B 40% isocratic elution; flow rate: 120 mL/min; back pressure: 100 bar; column temperature: 25° C.; wavelength: 220 nm.

Compound 42-1: retention time: 1.217 min. ¹H NMR (400 MHz, DMSO-d6) δ 9.60 (s, 1H), 8.48 (s, 1H), 6.85 (d, 1H), 6.41-6.34 (m, 2H), 4.03-3.87 (m, 2H), 3.74-3.56 (m, 2H), 3.53-3.41 (m, 3H), 3.28-3.13 (m, 5H), 3.11-2.96 (m, 6H).

LCMS (ESI): m/z=434.2 [M+H]⁺

Compound 42-2: retention time: 1.570 min. ¹H NMR (400 MHz, DMSO-d6) δ 9.60 (s, 1H), 8.48 (s, 1H), 6.85 (d, 1H), 6.41-6.34 (m, 2H), 4.03-3.87 (m, 2H), 3.74-3.56 (m, 2H), 3.53-3.41 (m, 3H), 3.28-3.13 (m, 5H), 3.11-2.96 (m, 6H).

LCMS (ESI): m/z=434.2 [M+H]⁺

### Example 44

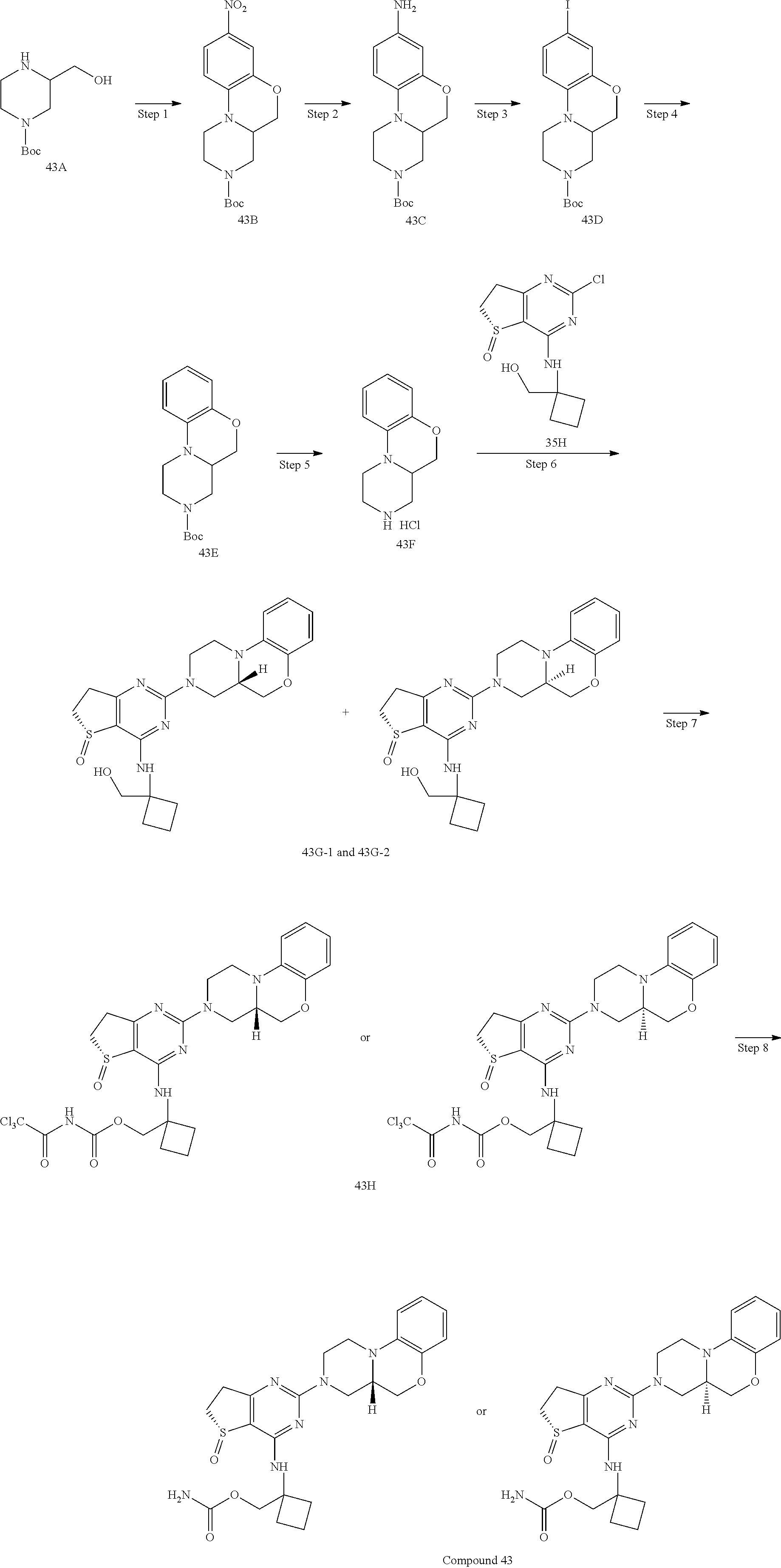

Step 1: To a 250 mL single-mouth flask, 43A (10.0 g, 46.3 mmol), 1,2-difluoro-4-nitrobenzene (7.3 g, 46.3 mmol), potassium hydroxide (7.8 g, 138.9 mmol), and N,N-dimethylformamide (100 mL) were successively added. After reaction at room temperature for 6 hours, the system was then heated to 60° C. and reacted for 24 hours. After filtration, the filtrate was added to ethyl acetate (500 mL) and washed with water (100 mL×3). The organic layers were dried over anhydrous sodium sulfate. After filtration, the filtrate was concentrated under reduced pressure and then separated and purified by silica gel column chromatography (eluents: ethyl acetate/petroleum ether (v/v)=20/100) to obtain 43B (10.0 g, yield 64.5%).

LC-MS (ESI): m/z=336.2 [M+H]⁺.

Step 2: To a 250 mL single-mouth flask, 43B (10.0 g, 29.9 mmol), ethyl acetate (100 mL), and palladium on carbon (2 g) were successively added, and the mixture was hydrogenated and reacted at room temperature for 12 hours. After filtration, the filtrate was concentrated under reduced pressure to obtain 43C (7.0 g, yield 76.9%).

LC-MS (ESI): m/z=306.2 [M+H]⁺.

Step 3: To a 100 mL single-mouth flask, acetonitrile (30 mL), tert-butyl nitrite (1.5 g, 14.7 mmol), and cuprous iodide (2.24 g, 11.8 mmol) were successively added and heated to 65° C., and a solution of 43C (3.0 g, 9.8 mmol) in acetonitrile (10 mL) was dropwise added. After the dropwise addition was complete, the system was reacted at 65° C. for 4 hours, and after heating was turned off, the system was further reacted for 12 hours, concentrated under reduced pressure, and then separated and purified by silica gel column chromatography (eluents: ethyl acetate/petroleum ether (v/v)=20/100) to obtain 43D (0.4 g, yield 12.6%).

LC-MS (ESI): m/z=361.2 [M−56+H]⁺.

Step 4: To a 50 mL single-mouth flask, methanol (10 mL), 43D (0.5 g, 1.20 mmol), and palladium on carbon (100 mg) were successively added, and the mixture was hydrogenated and reacted at room temperature for 16 hours. After filtration, the filtrate was concentrated under reduced pressure to obtain 43E (0.3 g, yield 86.1%).

LC-MS (ESI): m/z=235.2 [M−56+H]⁺.

Step 5: To a 50 mL single-mouth flask, 43E (0.3 g, 1.03 mmol) and a hydrogen chloride-dioxane solution (4N, 15 mL) were successively added and reacted at room temperature for 1 hour, and the system was concentrated under reduced pressure to obtain 43F (0.27 g, yield 99.6%).

LC-MS (ESI): m/z=191.2 [M+H]⁺.

Step 6: To a 50 mL single-mouth flask, 35H (287 mg, 1.0 mmol), 43F (270 mg, 1.03 mmol), dioxane (10 mL), and DIPEA (774 mg, 6.0 mmol) were successively added and reacted at 100° C. for 12 hours. After cooling to room temperature, the reaction liquid was directly concentrated under reduced pressure and then separated and purified by silica gel column chromatography (eluents: dichloromethane/methanol (v/v)=100/8) to obtain 400 mg of a mixture.

Chiral preparation was carried out to obtain the title compound 43G-1 (62 mg, 14.1%, retention time: 1.733 min) and the title compound 43G-2 (51 mg, 11.5%, retention time: 1.884 min). Chiral preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 100 mL/min; back pressure: 100 bar; column temperature: 25° C.; wavelength: 220 nm; elution time: 3.0 min.

### (Compound 43G-1)

¹H NMR (400 MHz, DMSO-d₆) δ 7.43 (s, 1H), 6.98-6.86 (m, 1H), 6.86-6.75 (m, 1H), 6.76-6.59 (m, 2H), 4.90-4.79 (m, 1H), 4.78-4.59 (m, 2H), 4.37-4.28 (m, 1H), 4.00-3.92 (m, 1H), 3.90-3.78 (m, 1H), 3.78-3.68 (m, 2H), 3.49-3.36 (m, 1H), 3.27-2.81 (m, 5H), 2.76-2.55 (m, 2H), 2.41-2.12 (m, 4H), 1.87-1.70 (m, 2H).

LC-MS (ESI): m/z=442.2 [M+H]⁺.

### (Compound 43G-2)

¹H NMR (400 MHz, DMSO-d₆) δ 7.44 (s, 1H), 6.95-6.89 (m, 1H), 6.84-6.77 (m, 1H), 6.75-6.62 (m, 2H), 4.89-4.78 (m, 1H), 4.79-4.59 (m, 2H), 4.39-4.28 (m, 1H), 4.02-3.81 (m, 2H), 3.79-3.68 (m, 2H), 3.51-3.39 (m, 1H), 3.26-3.16 (m, 1H), 3.14-2.83 (m, 4H), 2.76-2.55 (m, 2H), 2.43-2.25 (m, 2H), 2.26-2.13 (m, 2H), 1.89-1.69 (m, 2H).

LC-MS (ESI): m/z=442.2 [M+H]⁺.

Step 7: To a 50 mL single-mouth flask, dichloromethane (5 mL) and 43G-1 (60 mg, 0.14 mmol) were successively added, trichloroacetyl isocyanate (52 mg, 0.28 mmol) was dropwise added at 0° C. and reacted at room temperature for 1 hour, and the system was concentrated under reduced pressure to obtain crude 43H, which was directly used in the next step.

LC-MS (ESI): m/z=629.1 [M+H]⁺.

Step 8: To a 50 mL single-mouth flask, crude 43H, methanol (2 mL), water (2 mL), and potassium carbonate (38 mg, 0.28 mmol) were successively added and reacted at room temperature overnight, and the reaction liquid was directly concentrated under reduced pressure and then separated and purified by silica gel column chromatography (eluents: dichloromethane/methanol (v/v)=100/8) to obtain the title compound 43 (40 mg, two-step yield 60.6%).

¹H NMR (400 MHz, DMSO-d₆) δ 7.69 (s, 1H), 7.00-6.86 (m, 1H), 6.88-6.75 (m, 1H), 6.74-6.59 (m, 2H), 6.49 (s, 2H), 4.79-4.61 (m, 2H), 4.49-4.14 (m, 3H), 4.03-3.76 (m, 2H), 3.50-3.37 (m, 1H), 3.27-3.15 (m, 1H), 3.14-2.84 (m, 4H), 2.76-2.54 (m, 2H), 2.43-2.18 (m, 4H), 1.90-1.73 (m, 2H).

LC-MS (ESI): m/z=485.2 [M+H]⁺.

### Example 45

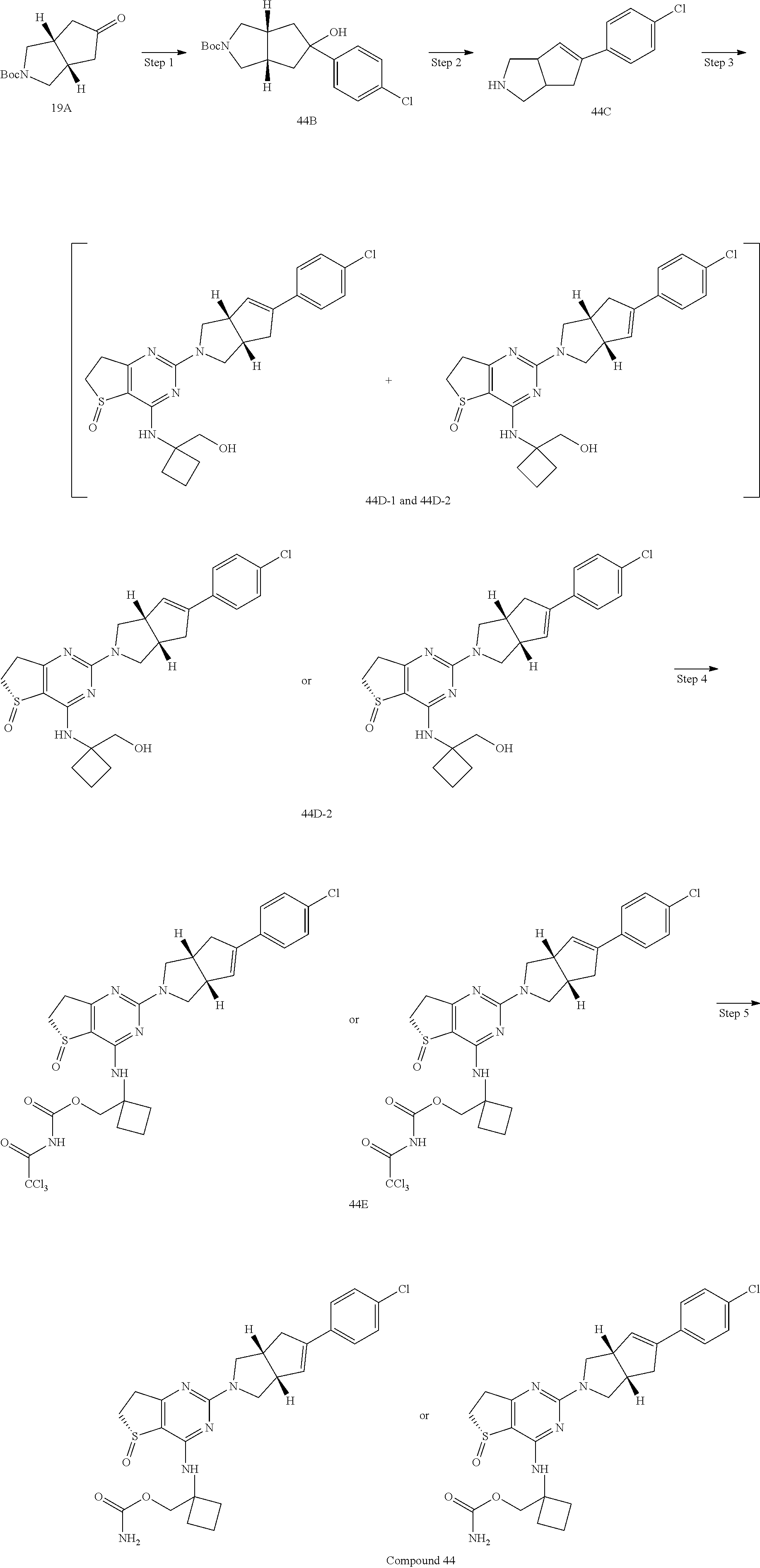

Step 1: p-Chloroiodobenzene (5.82 g, 24.41 mmol) and anhydrous tetrahydrofuran (50 mL) were added to a 250 ml three-mouth flask. After nitrogen displacement, n-butyllithium (39 mmol, 11.5 ml) was dropwise added at −78° C. and reacted at −78° C. for 2 h. A solution of 19A (5 g, 22.19 mmol) in tetrahydrofuran (20 ml) was then dropwise added, and the mixture was maintained at −78° C. and reacted for 1 h. Water (100 ml) was added, and the mixture was extracted with ethyl acetate (50 ml×2). The organic phases were combined, washed with a saturated sodium chloride solution, and dried over anhydrous sodium sulfate. After filtration, the filtrate was concentrated under reduced pressure and then separated and purified by silica gel column chromatography (eluents EA/PE=0-35%) to obtain the target compound 44B (4 g, 53.36%).

LC-MS (ESI): m/z=338.1 [M+H]⁺.

Step 2: 44B (4 g, 11.84 mmol) was added to a mixed system of dichloromethane (45 mL) and trifluoroacetic acid (15 mL) and reacted at room temperature for 3 h. The reaction liquid was directly concentrated under reduced pressure and then brought to the next reaction.

LC-MS (ESI): m/z=220.1 [M+H]⁺.

Step 3: To a 100 mL single-mouth flask, 35H (0.5 g, 1.74 mmol), 44C (0.7 g, 2.09 mmol), dioxane (20 mL), and DIPEA (1.12 g, 8.67 mmol) were successively added and reacted at 100° C. for 5 h. After cooling to room temperature, the reaction liquid was directly concentrated under reduced pressure and then separated and purified by silica gel column chromatography (eluents: MeOH/DCM=0-10%) to obtain 700 mg of a mixture.

Chiral preparation was carried out to obtain the title compound 44D-1 (300 mg, 36.6%, retention time: 0.786 min) and the title compound 44D-2 (240 mg, 29.3%, retention time: 0.993 min). Chiral preparation method: instrument: Waters 150 MGM; chromatographic column: Chiralpak Column; mobile phase: A: carbon dioxide, and B: methanol (0.1% aqueous ammonia); isocratic elution: 30% mobile phase B; flow rate: 120 mL/min; back pressure: 100 bar; column temperature: 25° C.; wavelength: 220 nm; elution time: 5.5 min.

Step 4: 44D-2 (0.1 g, 0.21 mmol) was added to dichloromethane (5 mL), the mixture was cooled to 0° C., and trichloroacetyl isocyanate (0.06 g, 0.32 mmol) was dropwise added. After reaction at 0° C. for 1 h, the reaction liquid was directly concentrated under reduced pressure to obtain a crude product (0.14 g), which was directly brought to the next reaction.

LC-MS (ESI): m/z=660.0 [M+H]⁺.

Step 5: Methanol (3 mL) and water (3 ml) were added to crude 44E (0.14 g), followed by potassium carbonate (0.09 g, 0.64 mmol). After reaction at room temperature for 16 h, water (10 ml) was added to the reaction liquid, and the mixture was extracted with dichloromethane (10 ml×2). The organic phases were concentrated under reduced pressure and then separated and purified by silica gel column chromatography (eluents: MeOH/DCM=0-10%) to obtain compound 44 (80 mg, 74%).

### (Compound 44)

¹H NMR (400 MHz, DMSO-d6) δ 7.53-7.35 (m, 5H), 6.47 (s, 2H), 6.26 (s, 1H), 4.47-4.30 (m, 2H), 3.95-3.91 (m, 1H), 3.77-3.53 (m, 3H), 3.46-3.34 (m, 1H), 3.23-3.01 (m, 3H), 2.97-2.78 (m, 3H), 2.67-2.57 (m, 1H), 2.45-2.09 (m, 4H), 1.91-1.72 (m, 2H).

LC-MS (ESI): m/z=514.1 [M+H]⁺.

### Example 46

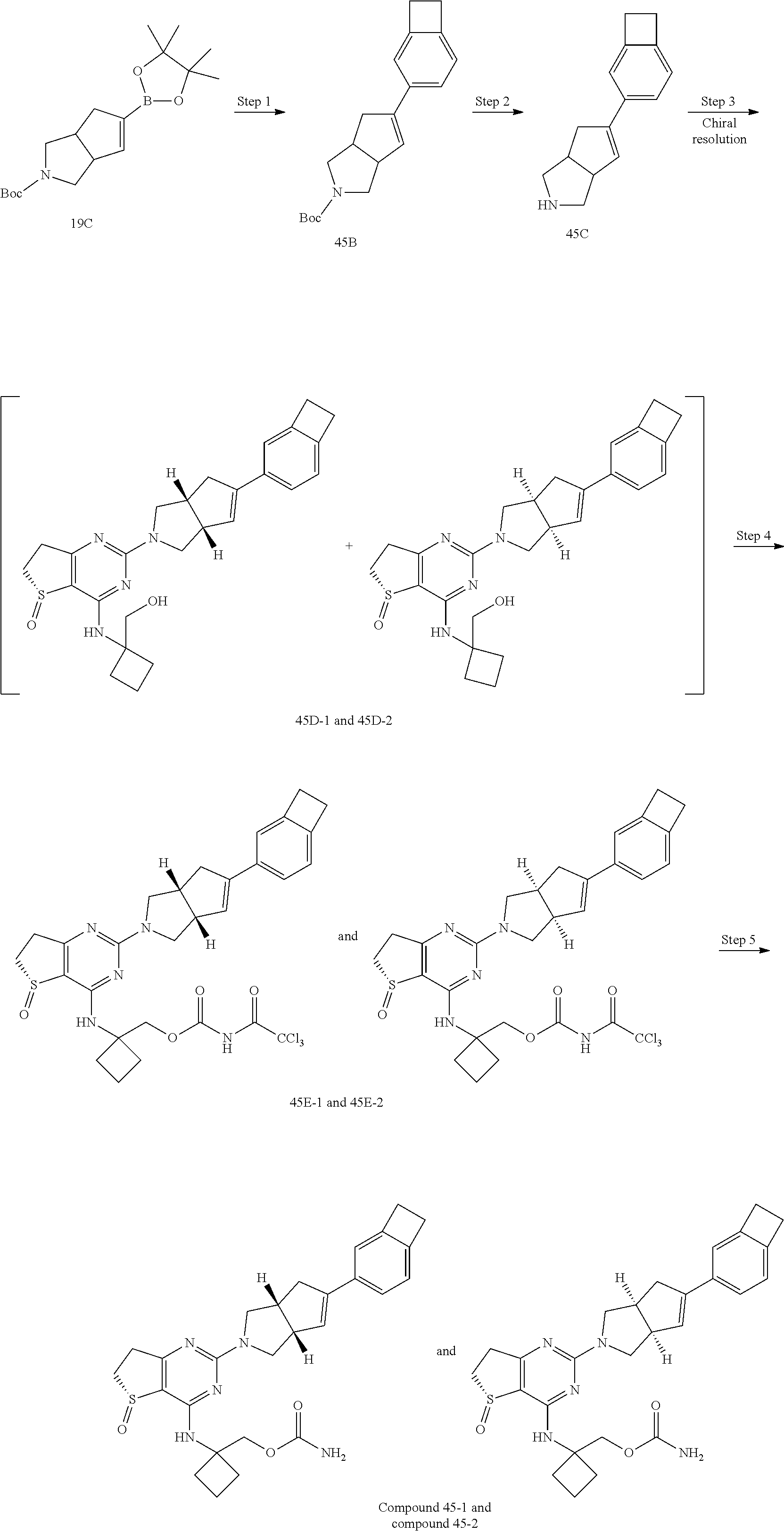

Using compound 19C and 4-bromobenzocyclobutane as raw materials, compound 45-1 (42 mg, 96%) and compound 45-2 (25 mg, 93%) were obtained according to the operation of Examples 20 and 36.

### Compound 45-1

LCMS (ESI): m/z=505.2 [M+H]⁺

¹H NMR (400 MHz, CDCl₃) δ=7.26-7.23 (m, 1H), 7.13 (s, 1H), 6.99 (d, 1H), 5.97 (s, 1H), 5.64 (s, 1H), 4.79 (s, 1H), 4.53 (s, 1H), 4.07-3.91 (m, 1H), 3.89-3.66 (m, 3H), 3.59 (s, 2H), 3.42-3.37 (m, 1H), 3.15 (s, 4H), 3.12-3.08 (d, 1H), 3.06-2.91 (m, 3H), 2.63 (d, 1H), 2.50-2.13 (m, 4H), 2.04-1.83 (m, 2H).

### Compound 45-2

LCMS (ESI): m/z=505.2 [M+H]⁺

¹H NMR (400 MHz, CDCl₃) δ=7.26-7.25 (m, 1H), 7.13 (s, 1H), 6.99 (d, 1H), 5.97 (s, 1H), 5.59 (s, 1H), 4.75 (s, 2H), 4.50 (t, 1H), 3.96 (s, 1H), 3.89-3.69 (m, 2H), 3.60 (s, 2H), 3.42-3.35 (m, 1H), 3.16 (s, 4H), 3.12-3.07 (m, 1H), 3.06-2.92 (m, 3H), 2.63 (d, 1H), 2.38 (s, 2H), 2.23 (s, 2H), 1.95-1.88 (m, 2H).

### Example 47

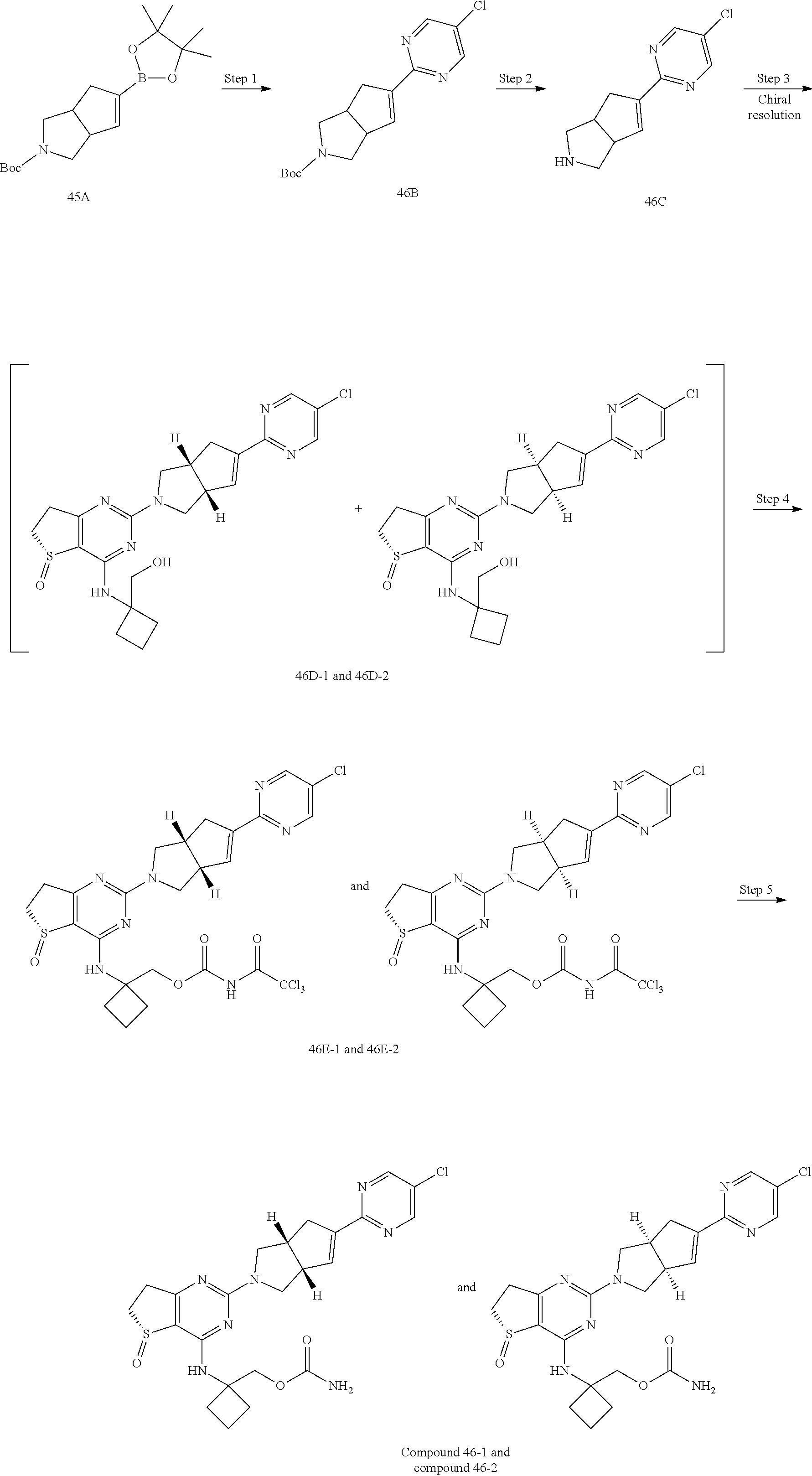

Using compound 19C and 5-chloro-2-iodopyrimidine as raw materials, compounds 46-1 (42 mg, 91%) and 46-2 (41 mg, 94%) were obtained according to the operation of Examples 20 and 36.

LCMS (ESI): m/z=515.2 [M+H]⁺

¹H NMR (400 MHZ, CDCl₃) δ=8.62 (s, 2H), 6.86 (s, 1H), 5.56 (s, 1H), 4.76 (s, 2H), 4.52-4.45 (m, 2H), 4.12-3.73 (m, 3H), 3.68 (s, 1H), 3.62-3.54 (m, 1H), 3.45-3.23 (m, 2H), 3.22-3.11 (m, 1H), 3.10-2.89 (m, 3H), 2.84 (d, 1H), 2.46-2.30 (m, 2H), 2.31-2.16 (m, 2H), 2.06-1.80 (m, 2H), 1.67 (s, 2H).

LCMS (ESI): m/z=515.2 [M+H]⁺

¹H NMR (400 MHz, CDCl₃) δ=8.62 (s, 2H), 6.86 (s, 1H), 5.58 (s, 1H), 4.72 (s, 2H), 4.56-4.47 (m, 2H), 4.13-3.53 (m, 5H), 3.45-3.36 (m, 1H), 3.32-3.13 (m, 2H), 3.10-2.97 (m, 3H), 2.86-2.82 (m, 1H), 2.42-2.35 (m, 2H), 2.24 (s, 2H), 1.98-1.86 (m, 2H), 1.62 (s, 2H).

### Example 48

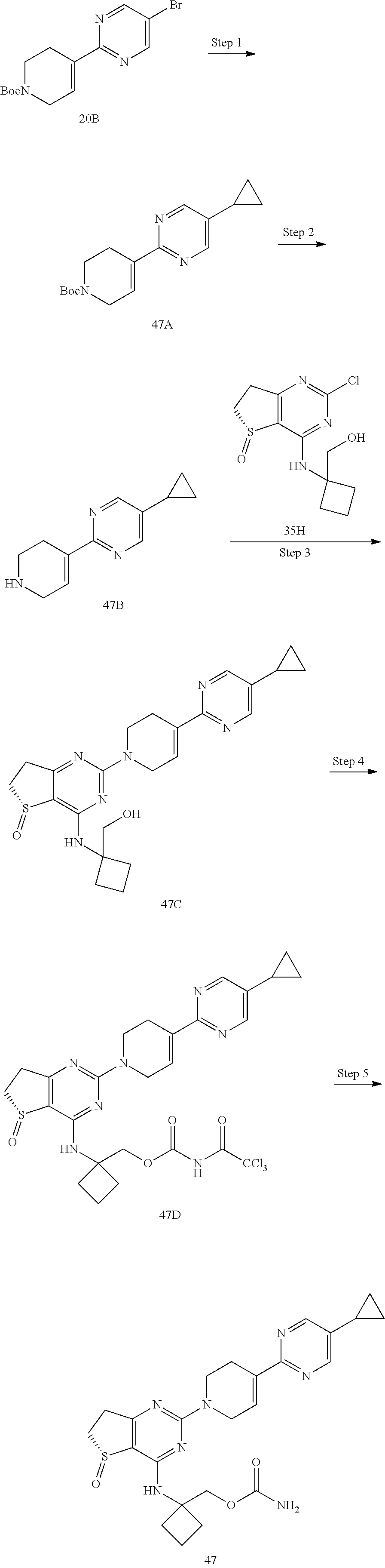

Step 1: Compound 20B (1.0 g, 3.0 mmol), cyclopropylboronic acid (356.0 mg, 4.0 mmol), palladium acetate (25.0 mg, 0.15 mmol), tricyclohexylphosphine (84.2 mg, 0.3 mmol), and potassium phosphate (2.3 g, 10.5 mmol) were added, and toluene (14 mL) and water (0.7 mL) were finally added. After nitrogen displacement three times, the mixture was heated to 100° C. and reacted for 3 h, and the system was then passed through a short silica gel column, eluted with ethyl acetate, concentrated, and subjected to column chromatography (petroleum ether/ethyl acetate=10/1) to obtain compound 47A (820.0 mg, 91%).

LC-MS (ESI): m/z=302.1 [M+H]⁺

Step 2: Compound 47A (820.0 mg, 2.7 mmol) was dissolved in dichloromethane (20 mL), trifluoroacetic acid (5.0 mL) was then added, and the mixture was reacted at room temperature for half an hour and then concentrated to obtain compound 47B (1.0 g, 99%), which was directly used for the next step.

LC-MS (ESI): m/z=202.1 [M+H]⁺.

Step 3: Compound 47B (400.0 mg, 2.0 mmol) was dissolved in 1,4-dioxane (10.0 mL), compound 35H (540.0 mg, 2.0 mmol) and diisopropylethylamine (1.0 g, 8.0 mmol) were then added, and the mixture was heated back to 90° C. and reacted for 12 h. The system was cooled to room temperature, concentrated, and subjected to column chromatography (dichloromethane/methanol=10/1) to obtain compound 47C (180.0 mg, 20%).

LC-MS (ESI): m/z=453.1 [M+H]⁺.

Step 4: Compound 47C (0.13 g, 0.28 mmol) was dissolved in dichloromethane (5 mL), trichloroacetyl isocyanate (64.0 mg, 0.34 mmol) was added in an ice bath, and the mixture was further reacted for 1 h. The system was concentrated to obtain 47D (0.18 g, 99%), which was directly used for the next step.

Step 5: Compound 47D (0.18 g, 0.28 mmol) was dissolved in methanol (2 mL), water (2 mL) and potassium carbonate (0.1 g, 0.8 mmol) were then added, and the mixture reacted at room temperature for 2.5 hours. After concentration, the system was extracted by adding dichloromethane, dried, and concentrated to obtain compound 47 (0.07 g, 51%).

LC-MS (ESI): m/z=496.2 [M+1]⁺

¹H NMR (400 MHz, DMSO-d6) δ 8.54 (s, 2H), 7.62 (s, 1H), 7.18 (s, 1H), 6.49 (s, 2H), 4.43-4.40 (m, 3H), 3.97 (s, 2H), 3.54-3.39 (m, 1H), 3.25-3.17 (m, 1H), 2.99-2.85 (m, 2H), 2.65 (s, 2H), 2.46-2.11 (m, 4H), 2.00-1.70 (m, 4H), 1.08-0.97 (m, 2H), 0.89-0.78 (m, 2H).

### Example 49

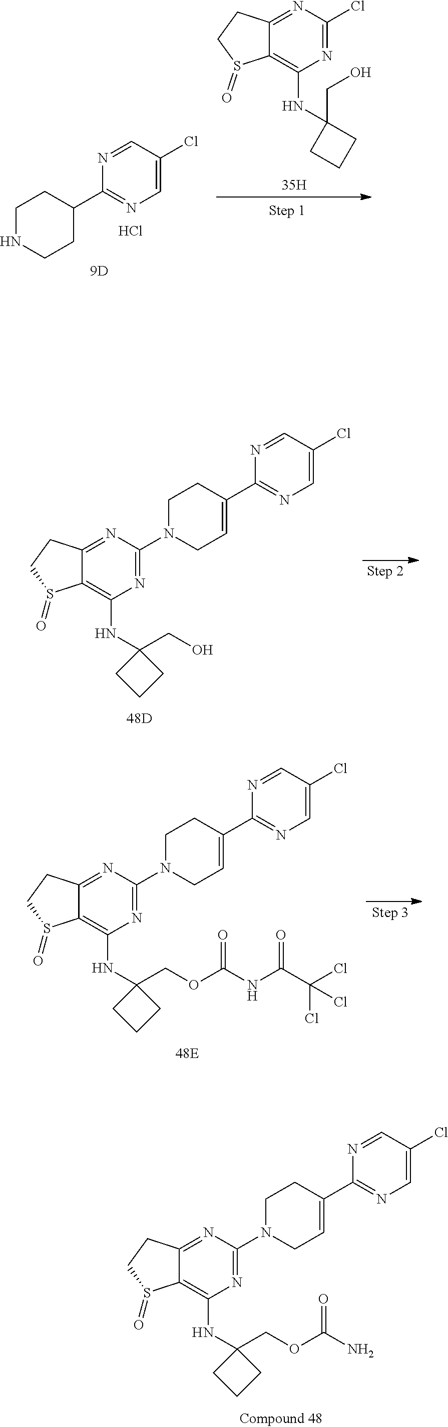 [SMILES: Clc1cnc(C2CCNCC2)nc1 ; Cl ; O=S1CCc2nc(Cl)nc(NC3(CO)CCC3)c21 ; O=[S@@]1CCc2nc(N3CC=C(c4ncc(Cl)cn4)CC3)nc(NC3(CO)CCC3)c21 ; O=C(NC(=O)C(Cl)(Cl)Cl)OCC1(Nc2nc(N3CC=C(c4ncc(Cl)cn4)CC3)nc3c2[S@](=O)CC3)CCC1 ; NC(=O)OCC1(Nc2nc(N3CC=C(c4ncc(Cl)cn4)CC3)nc3c2[S@](=O)CC3)CCC1]

Step 1: To a 50 mL single-mouth flask, 35H (0.20 g, 0.69 mmol) was added and dissolved with dioxane (10 mL), 9D (0.21 g, 0.92 mmol) and diisopropylethylamine (0.26 g, 2.05 mmol) were added, and the mixture was reacted at 90 degrees Celsius overnight. The reaction liquid was concentrated, purified by HPLC, and freeze-dried to obtain the title compound 48D (130 mg, 42%).

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A with a content of 30-70%; c. flow: 15 mL/min; d. elution time: 15 min; retention time: 8.0 min.

¹H NMR (400 MHz, DMSO-d6) δ 8.89 (s, 2H), 7.34 (s, 1H), 7.31-7.26 (m, 1H), 4.83 (t, 1H), 4.46 (s, 2H), 3.97 (t, 2H), 3.75 (d, 2H), 3.45-3.41 (m, 1H), 3.24-3.21 (m, 1H), 2.98-2.83 (m, 2H), 2.65 (s, 2H), 2.43-2.28 (m, 2H), 2.25-2.14 (m, 2H), 1.90-1.73 (m, 2H).

LC-MS (ESI): m/z=447.1 [M+H]⁺.

Step 2: To a 50 mL single-mouth flask, 48D (60 mg, 0.13 mmol) was added and dissolved with anhydrous dichloromethane (15 mL), trichloroacetyl isocyanate (30 mg, 0.16 mmol) was added under ice bath condition, and the system was maintained under the ice bath condition and stirred for 4 h. After the reaction was complete as monitored by TLC (DCM:MeOH=10:1), the reaction liquid was concentrated to obtain the title compound 48E (85 mg, crude), which was directly used for the next reaction.

Step 3: To a 50 mL single-mouth flask, 48E (85 mg, 0.13 mmol) was added and dissolved with methanol (6 mL), and potassium carbonate (54 mg, 0.39 mmol) was added and stirred at room temperature for 6 h. The reaction liquid was concentrated and separated by SFC to obtain the target compound 48 (30 mg, 47%).

Preparation method: instrument: SFC Prep 150; chromatographic column: torus AP (19 mm×250 mm); the sample was dissolved with MeOH and filtered with a 0.45 μm filter to prepare a sample liquid; preparative chromatography conditions: a. composition of mobile phases A and B: mobile phase A: CO₂, mobile phase B: MeOH; b. isocratic elution, mobile phase B with a content of 20%; c. flow: 40 mL/min; d. elution time: 20 min; retention time: 8.5 min.

LCMS m/z=490.2 [M+1]⁺

¹H NMR (400 MHz, DMSO-d6) δ 8.89 (s, 2H), 7.64 (s, 1H), 7.29 (s, 1H), 6.50 (s, 2H), 4.57-4.32 (m, 4H), 3.98 (t, 2H), 3.51-3.38 (m, 1H), 3.26-3.15 (m, 1H), 3.03-2.81 (m, 2H), 2.65 (s, 2H), 2.41-2.23 (m, 4H), 1.90-1.74 (m, 2H).

### Example 50

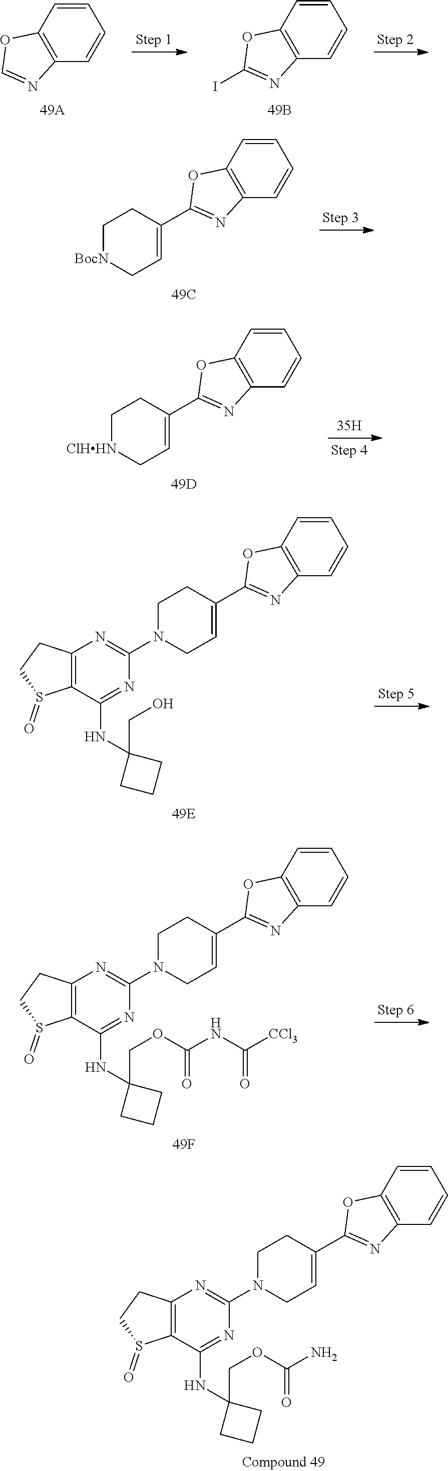

Step 1: Under nitrogen protection, 49A (1.00 g, 8.39 mmol) was dissolved in tetrahydrofuran (30 mL), and after the solution was cooled to −78° C., n-butyllithium (810 mg, 12.59 mmol) was slowly added. After the addition was complete, the reaction liquid was heated to −40° C., stirred for 1 h, and then cooled to −78° C., and a solution of iodine (2.56 g, 10.07 mmol) in tetrahydrofuran (10 mL) was added. The system then naturally returned to room temperature and was stirred for 1 h, the reaction was quenched by adding water (100 mL), and the system was extracted three times with ethyl acetate (100 mL). The organic phases were combined, dried over anhydrous sodium sulfate, and then concentrated in vacuum to obtain a crude product, and the crude product was purified by column chromatography (eluents: PE:EA=100:1 to 20:1) to obtain the target compound 49B (700 mg, yield: 34.03%).

¹H NMR (400 MHz, DMSO-d6) δ 7.76-7.73 (m, 2H), 7.38-7.36 (m, 2H).

Using compounds 49B and 9B as raw materials, compound 49 (15 mg, 46.21%) was obtained according to the operation of Examples 10 and 36.

LCMS m/z=495.10 [M+H]⁺

¹H NMR (400 MHz, DMSO-d6) δ 7.73-7.69 (m, 2H), 7.67 (s, 1H), 7.41-7.34 (m, 2H), 7.10 (s, 1H), 6.49 (s, 2H), 4.51 (s, 2H), 4.44-4.38 (m, 2H), 4.03-4.00 (m, 2H), 3.49-3.41 (m, 1H), 3.26-3.19 (m, 1H), 3.01-2.94 (m, 1H), 2.91-2.86 (m, 1H), 2.69 (s, 2H), 2.44-2.25 (m, 4H), 1.88-1.82 (m, 2H).

### Example 51

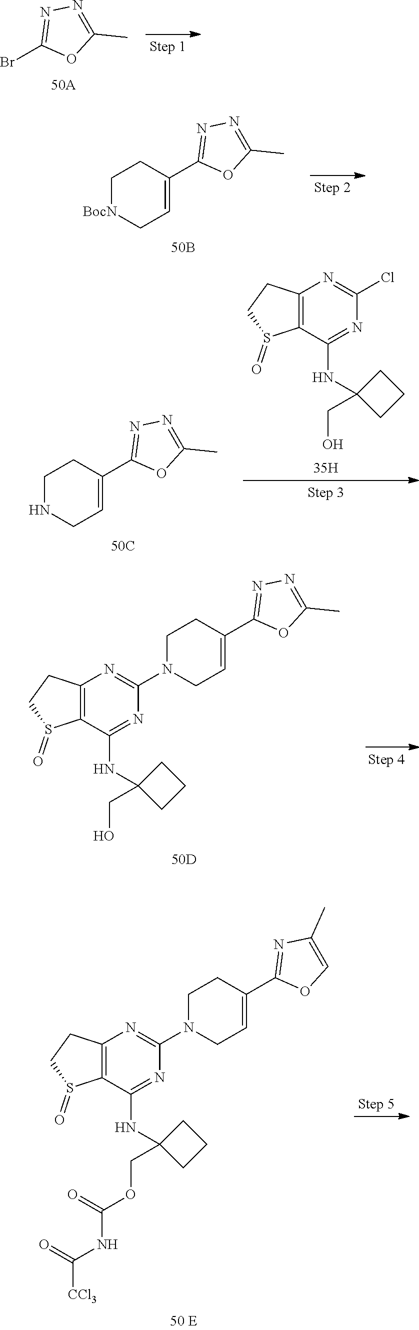

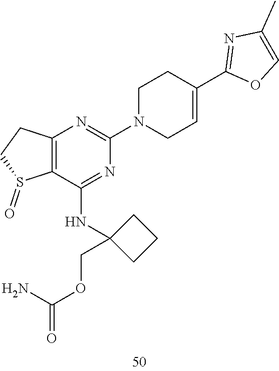 [SMILES: Cc1coc(C2=CCN(c3nc4c(c(NC5(COC(N)=O)CCC5)n3)[S@](=O)CC4)CC2)n1]

Using compounds 50A and 9B as raw materials, compound 50 (20 mg, 26%) was obtained according to the operation of Example 50.

LC-MS (ESI): m/z=459.2 [M+H]⁺.

¹H NMR (400 MHz, DMSO-d₆) δ 7.75 (d, 1H), 7.63 (s, 1H), 6.76-6.63 (m, 1H), 6.48 (s, 2H), 4.42-4.34 (m, 4H), 3.94 (t, 2H), 3.49-3.38 (m, 1H), 3.26-3.16 (m, 1H), 3.03-2.82 (m, 2H), 2.54 (s, 2H), 2.45-2.18 (m, 4H), 2.09 (d, 3H), 1.91-1.77 (m, 2H).

### Example 52

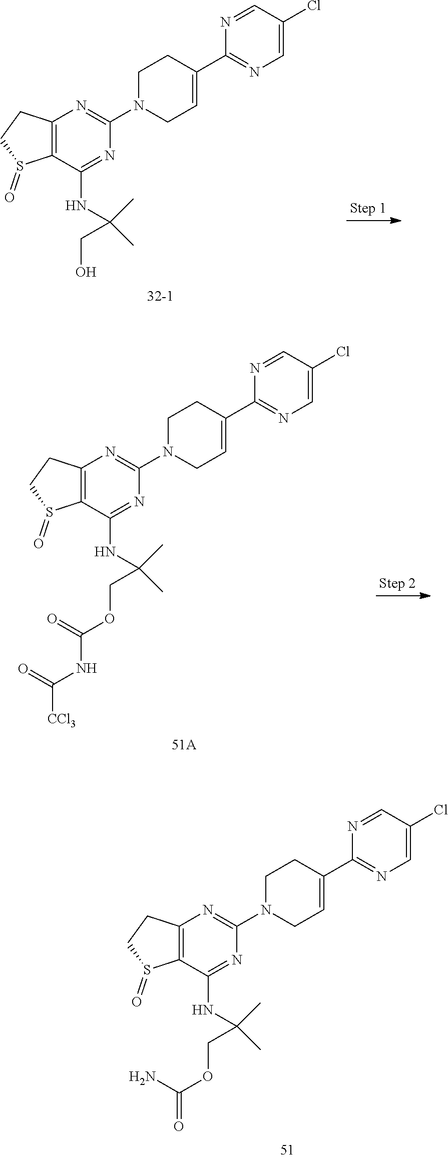

Step 1: Compound 32-1 (100 mg, 0.23 mmol) was dissolved in dichloromethane (5 mL), and trichloroacetyl isocyanate (52 mg, 0.28 mmol) was added in an ice bath and stirred in the ice bath for one hour. The reaction liquid was concentrated to obtain compound 51A (110 mg, 70%).

Step 2: Compound 51A (110 mg, 0.18 mmol) was dissolved in methanol (5 mL), and potassium carbonate (73 mg, 0.53 mmol) and water (5 mL) were added in an ice bath and stirred at room temperature 2.5 hours. The reaction liquid was diluted by adding water and extracted with dichloromethane, and the organic phases were combined, dried, filtered, concentrated, and subjected to silica gel column chromatography (dichloromethane:methanol 10:1) to obtain compound 51 (40 mg, 46%).

LC-MS (ESI): m/z=478.1 [M+H]⁺.

¹H NMR (400 MHz, DMSO-d₆) δ 8.89 (s, 2H), 7.29 (s, 1H), 6.70-6.40 (m, 3H), 4.50 (s, 2H), 4.30 (s, 2H), 4.01 (t, 2H), 3.49-3.37 (m, 1H), 3.30-3.23 (m, 1H), 3.02-2.92 (m, 1H), 2.91-2.82 (m, 1H), 2.67 (s, 2H), 1.46 (d, 6H).

### Example 53

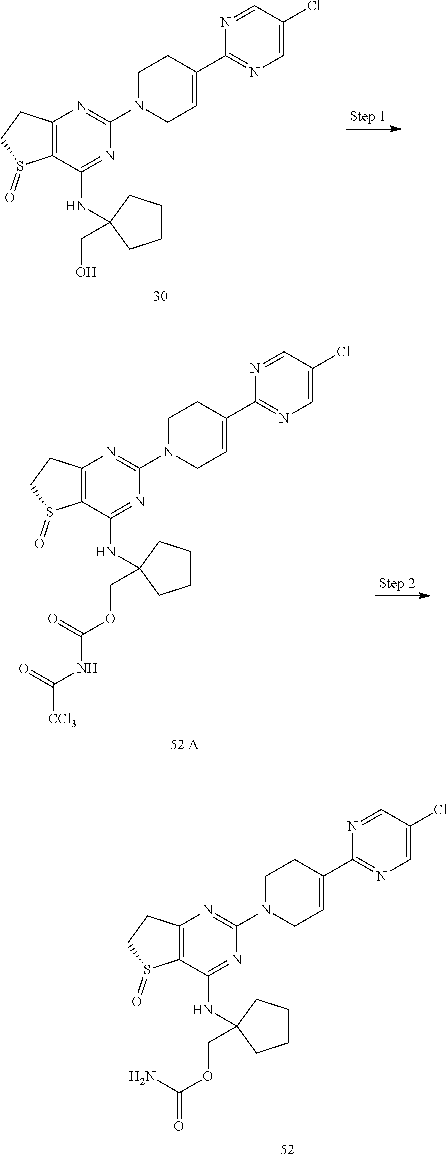

Step 1: Compound 30 (100 mg, 0.22 mmol) was dissolved in dichloromethane (5 mL), and trichloroacetyl isocyanate (52 mg, 0.28 mmol) was added in an ice bath and stirred in the ice bath for one hour. The reaction liquid was concentrated to obtain compound 52A (110 mg, 77%).

Step 2: Compound 52A (110 mg, 0.18 mmol) was dissolved in methanol (5 mL), and potassium carbonate (73 mg, 0.53 mmol) and water (5 mL) were added in an ice bath and stirred at room temperature 2.5 hours. The reaction liquid was diluted by adding water and extracted with dichloromethane, and the organic phases were combined, dried, filtered, concentrated, and subjected to silica gel column chromatography (dichloromethane:methanol 10:1) to obtain compound 52 (40 mg, 44%).

LC-MS (ESI): m/z=504.1 [M+H]⁺.

¹H NMR (400 MHz, DMSO-d₆) δ 8.90 (s, 2H), 7.34-7.20 (m, 1H), 6.84 (s, 1H), 6.47 (s, 2H), 4.49 (d, 2H), 4.46-4.31 (m, 2H), 4.00 (t, 2H), 3.48-3.38 (m, 1H), 3.29-3.20 (m, 1H), 3.02-2.84 (m, 2H), 2.66 (s, 2H), 2.25-2.10 (m, 2H), 1.95-1.79 (m, 2H), 1.70-1.56 (m, 4H).

### Example 54

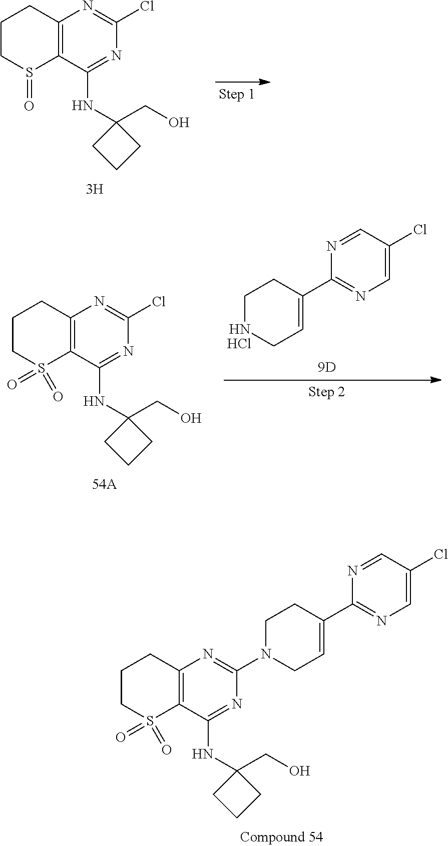 [SMILES: O=S1CCCc2nc(Cl)nc(NC3(CO)CCC3)c21 ; O=S1(=O)CCCc2nc(Cl)nc(NC3(CO)CCC3)c21 ; Cl ; Clc1cnc(C2=CCNCC2)nc1 ; O=S1(=O)CCCc2nc(N3CC=C(c4ncc(Cl)cn4)CC3)nc(NC3(CO)CCC3)c21]

Step 1: To a 50 mL single-mouth flask, compound 3H (500 mg, 1.66 mmol), dichloromethane (10 mL), m-chloroperoxybenzoic acid (342 mg, 1.99 mmol), and N,N-dimethylformamide (100 mL) were successively added. After reaction at room temperature for 2 hours, dichloromethane (20 mL) was added, and the mixture was washed with water (30 mL×2) and with a saturated sodium bicarbonate aqueous solution (30 mL×1). The organic layer was dried over anhydrous sodium sulfate. After filtration, the filtrate was concentrated under reduced pressure to obtain 54B (480 mg, yield 91.2%).

LC-MS (ESI): m/z=318.1 [M+H]⁺.

Step 2: To a 50 mL single-mouth flask, 54A (240 mg, 0.75 mmol), 9D (173 mg, 0.75 mmol), dioxane (10 mL), and DIPEA (484 mg, 3.75 mmol) were successively added and reacted at 100° C. for 12 hours. After cooling to room temperature, the reaction liquid was directly concentrated under reduced pressure and then separated and purified by silica gel column chromatography (eluents: dichloromethane/methanol (v/v)=100/8) to obtain the title compound 54 (42 mg, yield 11.7%).

¹H NMR (400 MHz, DMSO-d₆) δ 8.89 (s, 2H), 7.28 (s, 1H), 6.73 (s, 1H), 5.01-4.90 (m, 1H), 4.44 (s, 2H), 4.02-3.86 (m, 2H), 3.77-3.66 (m, 2H), 3.47-3.38 (m, 2H), 2.77-2.69 (m, 2H), 2.64 (s, 2H), 2.41-2.29 (m, 2H), 2.26-2.11 (m, 4H), 1.94-1.72 (m, 2H).

LC-MS (ESI): m/z=477.1 [M+H]⁺.

### Example 55

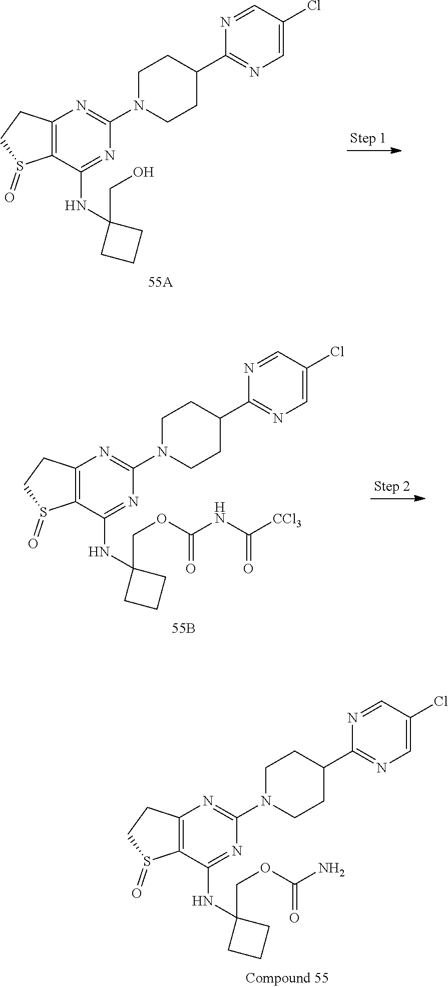

Step 1: Under nitrogen protection, compound 55A (100 mg, 0.22 mmol, see patent WO 2014124860 A1 for the synthesis) was dissolved in dichloromethane (10 mL), and trichloroacetyl isocyanate (50 mg, 0.27 mmol) was slowly dropwise added in an ice bath under stirring. After the addition was complete, the reaction liquid was stirred at 0° C. for 1 h. After the raw material disappeared as detected by a spotting plate (developing agent: DCM/MeOH=10:1), the reaction liquid was concentrated under reduced pressure to obtain crude product 55B (160 mg), which was directly used in the next reaction.

Step 2: Crude compound 55B (140 mg, 0.22 mmol) was dissolved in methanol (6 mL), and potassium carbonate (90 mg, 0.66 mmol) was then added at room temperature. After the addition was complete, the mixture was stirred at room temperature 1.5 h under nitrogen protection. After the reaction was complete, the reaction liquid was concentrated under reduced pressure to obtain a crude product, and the crude product was purified by column chromatography (eluents: DCM:MeOH=100:1 to 9:1), followed by further purification through a reverse phase column (eluents: water:acetonitrile=100:1 to 1:9) to obtain the target compound 55 (95 mg, 87.91%).

LCMS m/z=492.10 [M+H]⁺

¹H NMR (400 MHz, DMSO-d6) δ 8.86 (s, 2H), 7.59 (s, 1H), 6.48 (s, 2H), 4.72-4.69 (m, 2H), 4.38-4.32 (m, 2H), 3.46-3.40 (m, 1H), 3.25-3.16 (m, 2H), 3.10-3.04 (m, 2H), 2.97-2.85 (m, 2H), 2.40-2.27 (m, 2H), 2.22-2.19 (m, 2H), 1.98-1.95 (m, 2H), 1.89-1.80 (m, 2H), 1.68-1.60 (m, 2H).

### Example 56

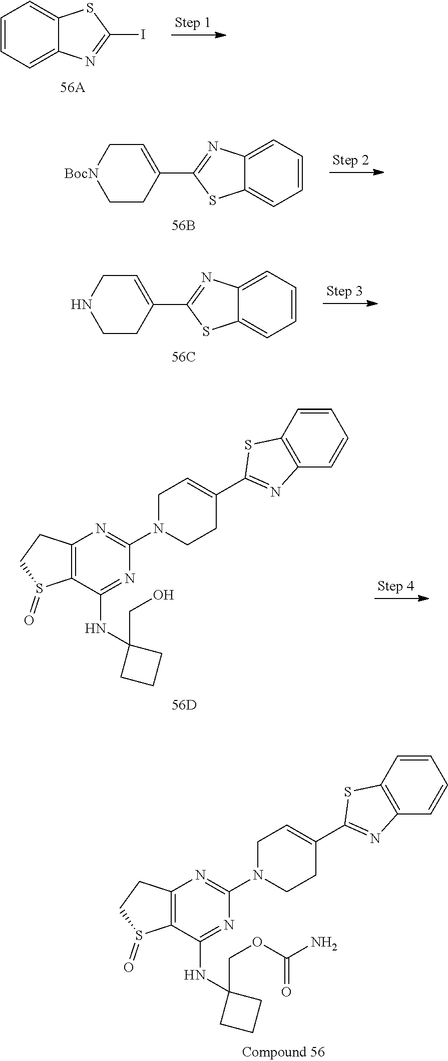 [SMILES: Ic1nc2ccccc2s1 ; CC(C)(C)OC(=O)N1CC=C(c2nc3ccccc3s2)CC1 ; C1=C(c2nc3ccccc3s2)CCNC1 ; O=[S@@]1CCc2nc(N3CC=C(c4nc5ccccc5s4)CC3)nc(NC3(CO)CCC3)c21 ; NC(=O)OCC1(Nc2nc(N3CC=C(c4nc5ccccc5s4)CC3)nc3c2[S@](=O)CC3)CCC1]

Using 56A and 9B as raw materials, compound 56 (5.2 mg, 6%) was obtained according to the operation of Example 50.

LCMS (ESI): m/z=511.0 [M+H]⁺

¹H NMR (400 MHz, DMSO-d6) δ 8.10-8.03 (m, 1H), 7.99-7.92 (m, 1H), 7.65 (s, 1H), 7.53-7.38 (m, 2H), 6.90 (s, 1H), 6.49 (s, 2H), 4.48-4.40 (m, 4H), 4.10-3.98 (m, 2H), 3.45-3.40 (m, 1H), 3.28-3.18 (m, 1H), 3.03-2.93 (m, 1H), 2.91-2.85 (m, 1H), 2.76 (s, 2H), 2.39-2.29 (m, 4H), 1.88-1.82 (m, 2H).

### Example 57

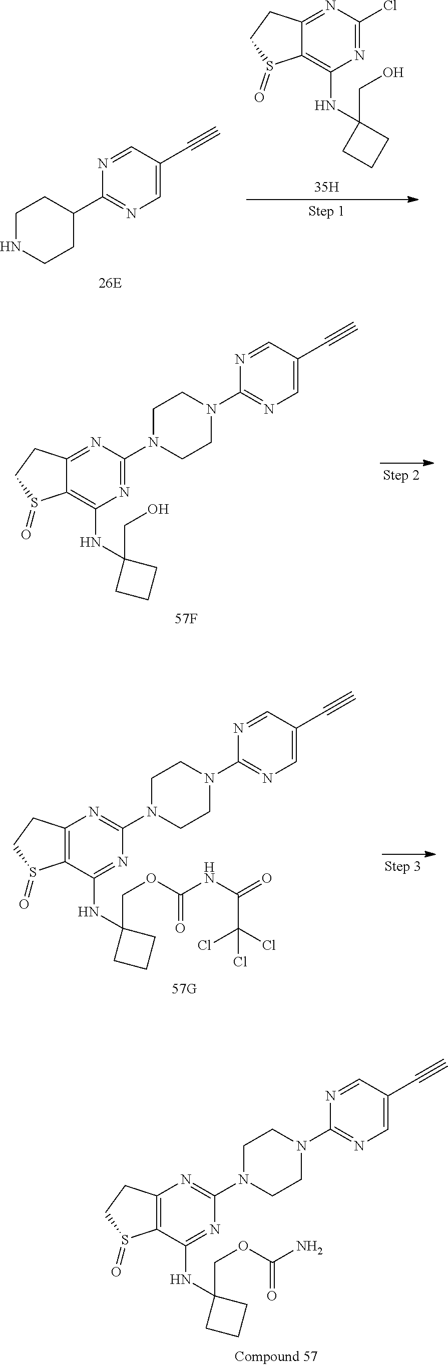

Using compounds 26E and 35H as raw materials, compound 57 (20 mg, 43%) was obtained according to the operation of Example 49.

LCMS m/z=483.20 [M+H]⁺

H NMR (400 MHZ, DMSO-d6) δ 8.50 (s, 2H), 7.66 (s, 1H), 6.49 (s, 2H), 4.32-4.40 (m, 2H), 4.28 (s, 1H), 3.83 (s, 8H), 3.40-3.48 (m, 1H), 3.18-3.26 (m, 1H), 2.84-2.99 (m, 2H), 2.21-2.41 (m, 4H), 1.76-1.88 (m, 2H).

### Example 58

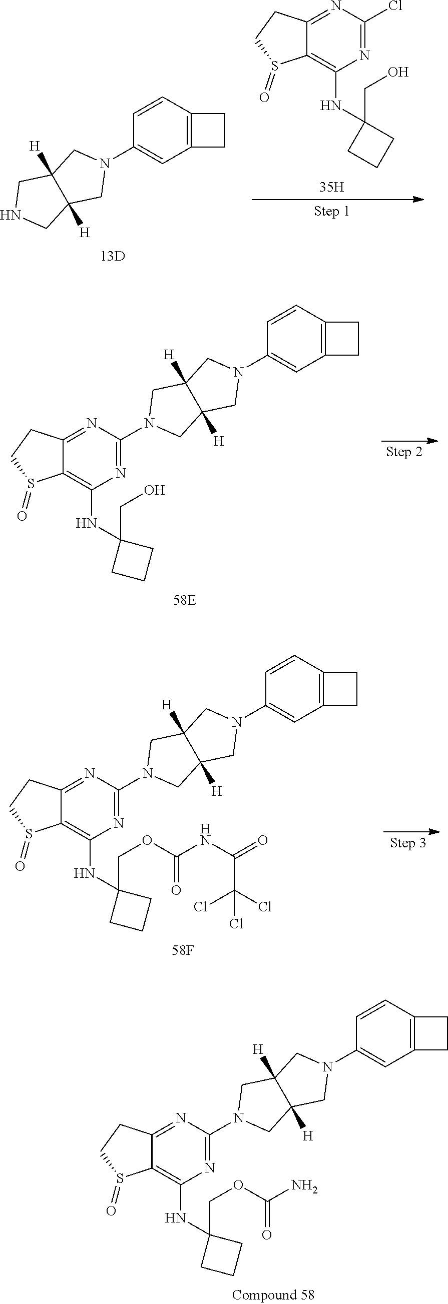

Using compounds 13D and 35H as raw materials, compound 58 (20 mg, 17%) was obtained according to the operation of Example 49.

LCMS m/z=509.20 [M+H]⁺

H NMR (400 MHZ, DMSO-d6) δ 7.50 (s, 1H), 6.85 (d, 1H), 6.47 (s, 2H), 6.34-6.36 (m, 2H), 4.38 (s, 2H), 3.74-3.82 (m, 2H), 3.40-3.42 (m, 4H), 3.01-3.21 (m, 9H), 2.83-2.95 (m, 2H), 2.27-2.47 (m, 2H), 2.16-2.23 (m, 2H), 1.76-1.84 (m, 3H).

### Example 59

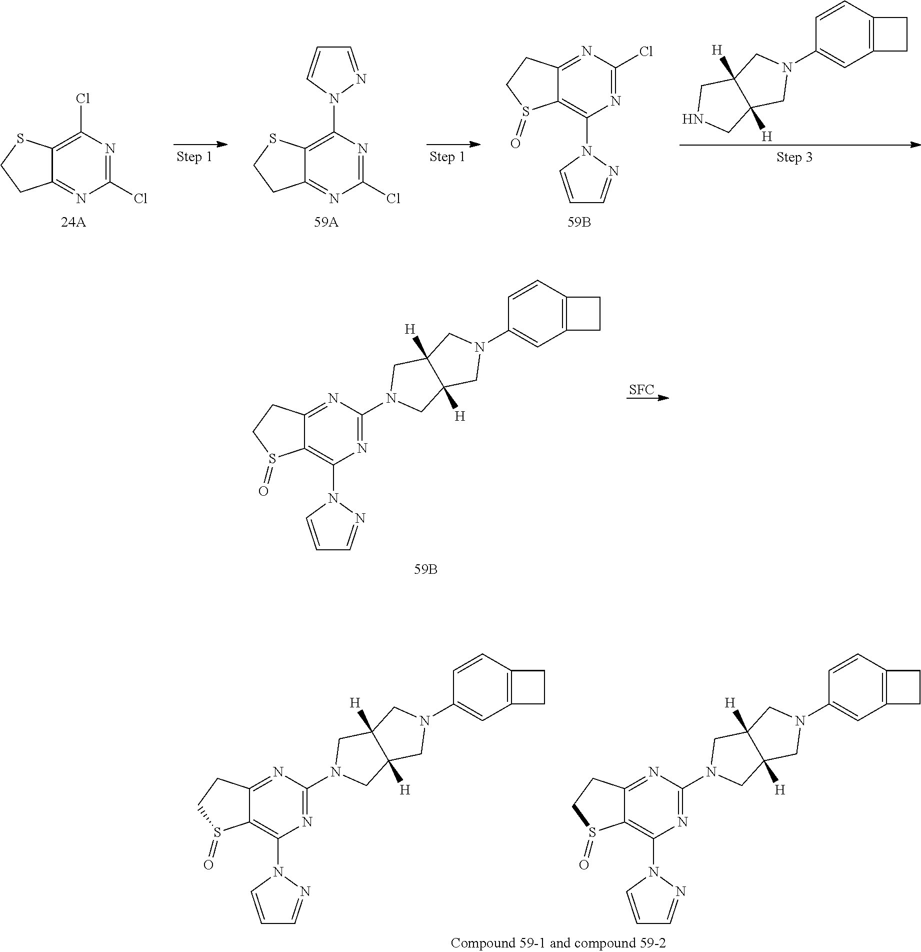

Using compound 24A and pyrazole as raw materials, compound 59-1 (30 mg) and compound 59-2 (50 mg) were obtained according to the operation of Example 41.

Chiral resolution method: instrument: Waters 150 SFC; column: Chiralpak IC Column (250*30 mm, I.D 30 mm, 10 μm particle size); mobile phase: A for CO₂ and B for IPA+ACN (0.1% NH₃·H₂O); gradient: 45% phase B isocratic elution; flow rate: 100 mL/min; back pressure: 100 bar; column temperature: 25° C.; wavelength: 220 nm; elution time: 9.3 min.

LCMS m/z=433.2 [M+1]⁺

Compound 59-1, retention time 2.01 min, ¹H NMR (400 MHZ, DMSO-d6) δ 8.75 (s, 1H), 8.02 (s, 1H), 6.85 (d, 1H), 6.68 (d, 1H), 6.36-6.38 (m, 2H), 3.86-4.01 (m, 2H), 3.55-3.71 (m, 3H), 3.40-3.50 (m, 2H), 3.11-3.26 (m, 6H), 2.97-3.05 (m, 5H).

Compound 59-2, retention time 2.48 min, ¹H NMR (400 MHZ, DMSO-d6) δ 8.76 (s, 1H), 8.02 (s, 1H), 6.85 (d, 1H), 6.67 (d, 1H), 6.36-6.38 (m, 2H), 3.86-4.01 (m, 2H), 3.55-3.71 (m, 3H), 3.41-3.49 (m, 2H), 3.11-3.24 (m, 6H), 2.97-3.05 (m, 5H).

### Example 60

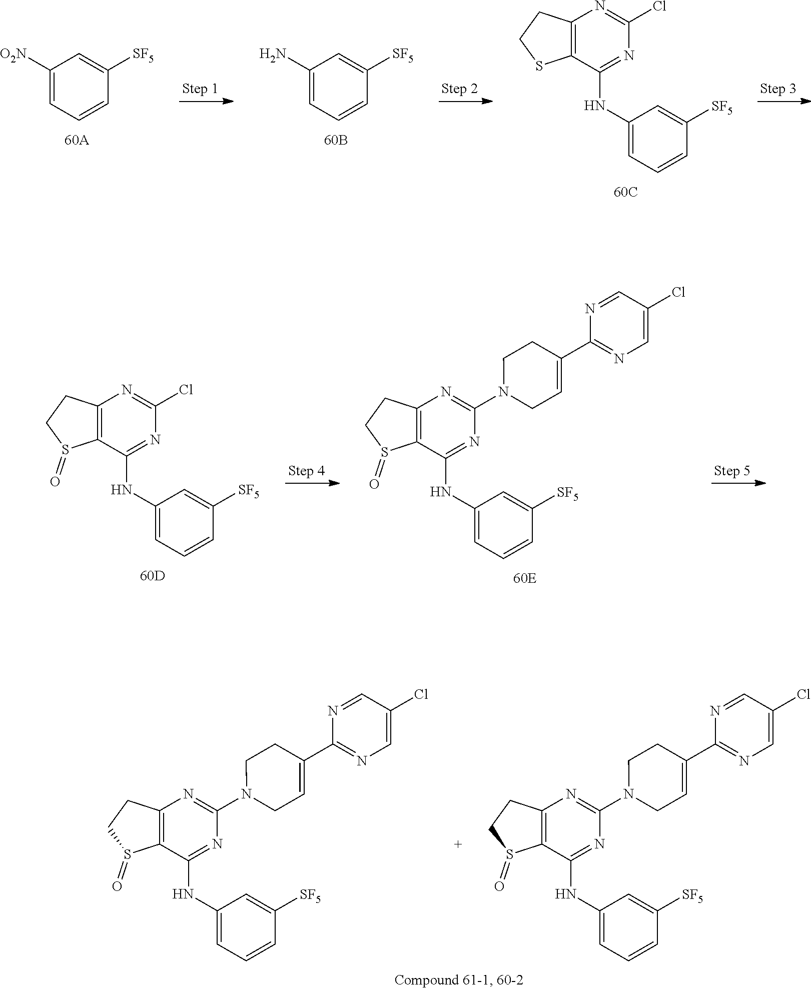

Step 1: At room temperature, 60A (5.00 g, 20.07 mmol) was dissolved in ethanol (100 mL), iron powder (6.72 g, 120.41 mmol) was added in portions, and concentrated hydrochloric acid (2.12 g, 20.37 mmol) was then slowly dropwise added. After the addition was complete, the reaction liquid was stirred at room temperature for 4 h under nitrogen protection. After the reaction was complete as monitored by TLC, the reaction was quenched by adding aqueous ammonia, and the system was filtered to obtain a filtrate. Water (100 mL) was added, and the system was diluted and extracted three times with dichloromethane (100 mL). The organic phases were combined, dried over anhydrous sodium sulfate, and then concentrated in vacuum to obtain a crude product, and the crude product was purified by column chromatography (eluents: PE:EA=100:1 to 4:1) to obtain the target compound 60B (3.00 g, yield: 68.21%).

LC-MS (ESI): m/z=220.20 [M+H]⁺.

¹H NMR (400 MHz, CDCl₃) δ 7.22-7.18 (m, 1H), 7.12-7.09 (m, 1H), 7.04-7.03 (m, 1H), 6.78-6.75 (m, 1H), 3.86 (br s, 2H).

Using compounds 60B and 24A as raw materials, compound 60-1 (100 mg) and compound 60-2 (160 mg) were obtained according to the operation of Example 37.

Analysis method: instrument: SHIMADZU LC-20AD, column: Chiralpak IG-3 50×4.6 mm I.D., 3 μm; mobile phase: A: heptane (0.05%, N,N-diethylaniline), B: ethanol (0.05% N,N-diethylaniline); gradient: 20% B in A; flow rate: 1 mL/min, column temperature: 35° C., wavelength: 254 nm.

Preparation method: instrument: SHIMADZU LC-20AP, column: DAICEL CHIRALPAK IG (250 mm×30 mm, 10 μm), mobile phase: A n-hexane, B ethanol (0.1% NH₃·H₂O); gradient: 8% B gradient elution, flow rate: 110 mL/min, column temperature: 25° C., wavelength: 254 nm, cycle time: 16 min, sample preparation: sample concentration 1.5 mg/ml, ethanol solution injection: 2 ml each time. After separation, the fraction was dried by a rotary evaporator at a bath temperature of 40° C. to obtain compound 60-1 (retention time: 5.716 minutes) and compound 60-2 (retention time: 6.890 minutes).

### Compound 60-1:

LCMS m/z=565.5 [M+H]⁺

¹H NMR (400 MHz, DMSO-d6) δ 9.98 (s, 1H), 8.90 (s, 2H), 8.60 (s, 1H), 7.98-7.90 (m, 1H), 7.63-7.60 (m, 2H), 7.31-7.24 (m, 1H), 4.54-4.42 (m, 2H), 4.08-3.96 (m, 2H), 3.62-3.54 (m, 1H), 3.36-3.28 (m, 1H), 3.15-3.09 (m, 1H), 3.04-2.99 (m, 1H), 2.74-2.65 (m, 2H).

### Compound 60-2:

LCMS m/z=565.5 [M+H]⁺

¹H NMR (400 MHz, DMSO-d6) δ 9.98 (s, 1H), 8.90 (s, 2H), 8.60 (s, 1H), 7.98-7.90 (m, 1H), 7.63-7.59 (m, 2H), 7.31-7.24 (m, 1H), 4.55-4.41 (m, 2H), 4.08-3.96 (m, 2H), 3.62-3.54 (m, 1H), 3.36-3.28 (m, 1H), 3.15-3.09 (m, 1H), 3.04-2.99 (m, 1H), 2.75-2.66 (m, 2H).

### Example 61

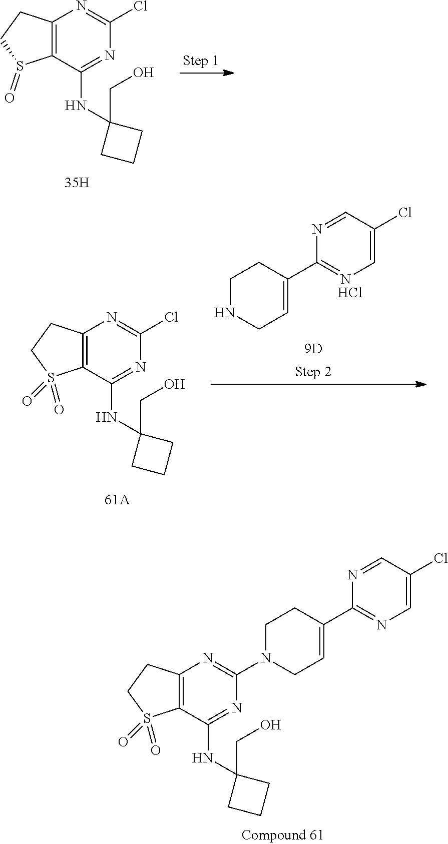 [SMILES: O=[S@@]1CCc2nc(Cl)nc(NC3(CO)CCC3)c21 ; O=S1(=O)CCc2nc(Cl)nc(NC3(CO)CCC3)c21 ; Clc1cnc(C2=CCNCC2)nc1 ; Cl ; O=S1(=O)CCc2nc(N3CC=C(c4ncc(Cl)cn4)CC3)nc(NC3(CO)CCC3)c21]

Step 1: To a 100 mL single-mouth flask, 35H (1.0 g, 3.47 mmol) was added and dissolved with dichloromethane (20 mL), and m-chloroperoxybenzoic acid (1.2 g, 6.94 mmol) was added and stirred at room temperature overnight. The reaction liquid was washed with a sodium hydroxide aqueous solution (5%, 20 mL×2), dried over anhydrous sodium sulfate, and concentrated to obtain the title compound 61A (1.0 g, 95%).

LC-MS (ESI): m/z=304.1 [M+H]⁺.

Step 2: To a 50 mL single-mouth flask, 61A (0.2 g, 0.66 mmol) was added and dissolved with dioxane (10 mL), and 9D (0.18 g, 0.79 mmol) and N,N-diisopropylethylamine (0.26 g, 1.98 mmol) were successively added and stirred at 95 degrees Celsius overnight. The reaction liquid was concentrated, slurried with methanol (10 mL) for 0.5 h, suction-filtrated, and dried to obtain the target compound 61 (0.25 g, 82%).

LC-MS (ESI): m/z=463.1 [M+H]⁺.

¹H NMR (400 MHz, DMSO-d6) δ 8.90 (s, 2H), 7.30-7.28 (m, 1H), 6.12 (s, 1H), 5.02 (t, 1H), 4.49 (s, 2H), 4.00 (t, 2H), 3.71 (d, 2H), 3.49-3.46 (m, 2H), 3.10-3.06 (m, 2H), 2.67 (s, 2H), 2.53-2.51 (m, 2H), 2.20-2.06 (m, 2H), 1.89-1.74 (m, 2H).

### Example 62

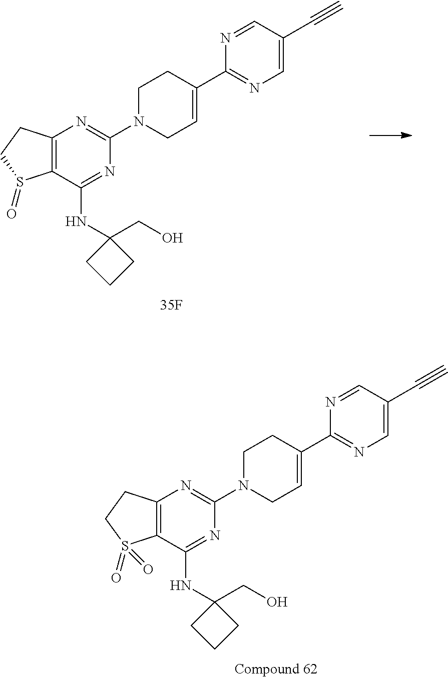 [SMILES: C#Cc1cnc(C2=CCN(c3nc4c(c(NC5(CO)CCC5)n3)[S@](=O)CC4)CC2)nc1 ; C#Cc1cnc(C2=CCN(c3nc4c(c(NC5(CO)CCC5)n3)S(=O)(=O)CC4)CC2)nc1]

Step 1: Compound 35F (860.0 mg, 2.0 mmol) was dissolved in dichloromethane (10 mL), m-chloroperoxybenzoic acid (346.0 mg, 2.0 mmol) was then added and reacted at room temperature for 4 h, and the system was quenched by adding a small amount of sodium bicarbonate, concentrated, and subjected to column chromatography (dichloromethane/methanol=10/1) to obtain compound 62 (120.0 mg, 12%).

LC-MS (ESI): m/z=453.1 [M+H]⁺.

¹H NMR (400 MHz, DMSO-d6) δ 8.91 (s, 2H), 7.34 (s, 1H), 6.12 (s, 1H), 5.02 (s, 1H), 4.70 (s, 1H), 4.51 (s, 2H), 4.00 (d, 2H), 3.71 (d, 2H), 3.48 (t, 2H), 3.08 (t, 2H), 2.68 (s, 2H), 2.53 (s, 2H), 2.14 (t, 2H), 1.97-1.68 (m, 2H).

### Example 63

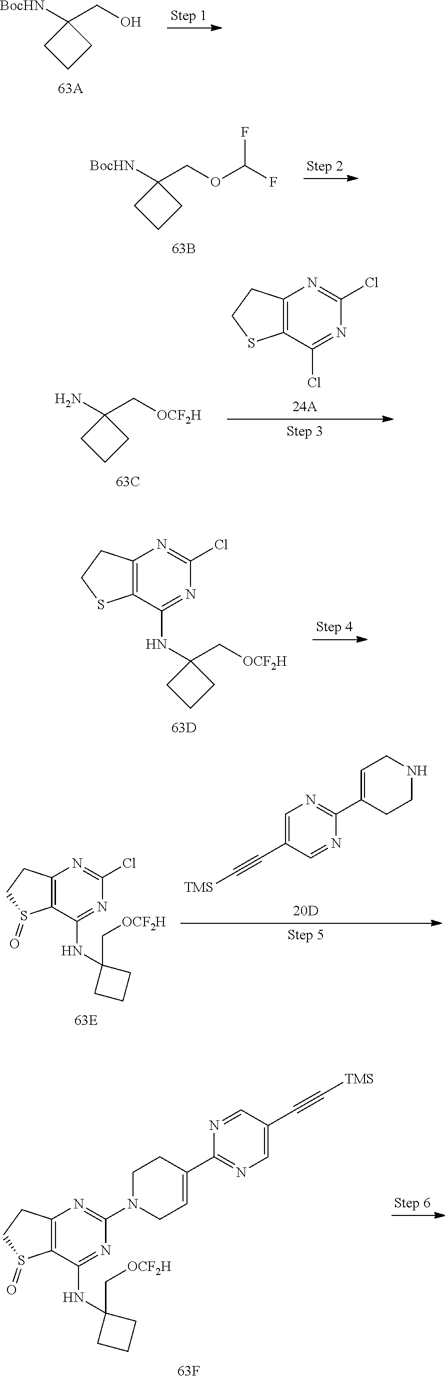

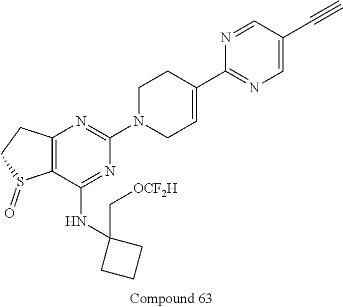

Step 1: Compound 63A (2.0 g, 10.0 mmol) was dissolved in dichloromethane (5 mL) and water (5 mL), potassium bifluoride (1.6 g, 20.0 mmol) was then added, and difluorobromomethyl trimethylsilane (2.0 g, 10.0 mmol) was finally added. The mixture was reacted at room temperature overnight, extracted with dichloromethane, dried, filtered, concentrated, and subjected to column chromatography (petroleum ether/ethyl acetate=10/1) to obtain compound 63B (2.0 g, 81%).

LC-MS (ESI): m/z=252.1 [M+H]⁺.

Step 2: Compound 63B (0.8 g, 3.1 mmol) was dissolved in dichloromethane (10 mL), trifluoroacetic acid (5.0 mL) was then added, and the mixture was reacted at room temperature for half an hour and then concentrated to obtain compound 63C (1.0 g, 99%), which was directly used for the next step.

LC-MS (ESI): m/z=152.1 [M+H]⁺.

Using compounds 63C and 24A as raw materials, compound 63 (35.0 mg, 28%) was obtained according to the operation of Example 34.

LC-MS (ESI): m/z=487.2 [M+1]⁺

¹H NMR (400 MHz, DMSO-d6) δ 8.90 (s, 2H), 7.82 (s, 1H), 7.34 (s, 1H), 6.70 (t, 1H), 4.69 (s, 1H), 4.46 (s, 2H), 4.27 (q, 2H), 3.96 (s, 2H), 3.54-3.36 (m, 1H), 3.24-3.17 (m, 1H), 3.00-2.85 (m, 2H), 2.66 (s, 2H), 2.45-2.18 (m, 4H), 1.90-1.84 (m, 2H).

¹⁹F NMR (377 MHz, DMSO-d6) δ −80.12 (s).

### Example 64

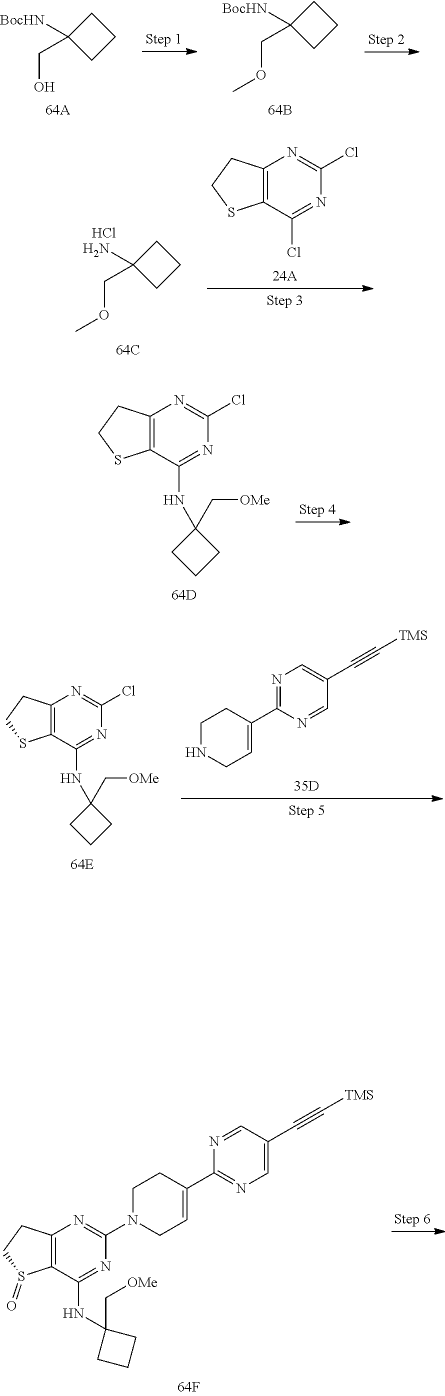

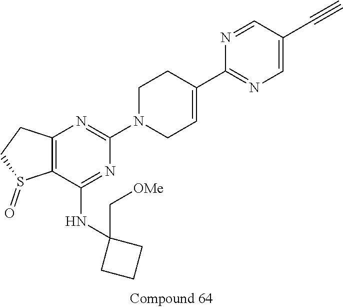 [SMILES: C#Cc1cnc(C2=CCN(c3nc4c(c(NC5(COC)CCC5)n3)[S@](=O)CC4)CC2)nc1]

Step 1: Compound 64A (5.0 g, 24.8 mmol) was dissolved in tetrahydrofuran (50 ml), sodium hydride (1.1 g, 27.5 mmol) was then added and stirred at room temperature for 1 h, and iodomethane (3.9 g, 27.5 mmol) was added and reacted for 12 h. The system was passed through a short silica gel column, eluted with ethyl acetate, concentrated, and subjected to column chromatography (ethyl acetate/petroleum ether=1/4) to obtain compound 64B (2.0 g, 38%).

Step 2: Compound 64B (2.0 g, 9.3 mmol) was dissolved in dichloromethane (30 mL), a hydrogen chloride dioxane solution (6.9 mL, 27.9 mmol) was then added and reacted at room temperature for half an hour, and the system was concentrated to obtain compound 64C (1.0 g, 92%), which was directly used for the next step.

LC-MS (ESI): m/z=116.2 [M+H]⁺

Using compounds 64C and 24A as raw materials, compound 64 (10.0 mg, 5.3%) was obtained according to the operation of Example 34.

LC-MS (ESI): m/z=451.2 [M+1]⁺

¹H NMR (400 MHz, DMSO-d6) δ 8.90 (s, 2H), 7.64 (s, 1H), 7.34 (s, 1H), 4.68 (s, 1H), 4.46 (s, 2H), 3.97 (q, 2H), 3.78-3.71 (m, 2H), 3.49-3.39 (m, 1H), 3.29 (s, 3H), 3.25-3.16 (m, 1H), 3.04-2.85 (m, 2H), 2.66 (s, 2H), 2.36-2.20 (m, 4H), 1.88-1.82 (m, 2H).

### Example 65

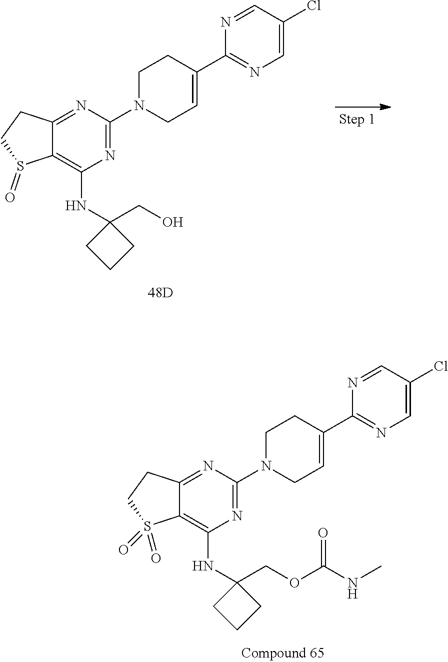

Step 1: To a 100 mL single-mouth flask, compound 48D (300 mg, 0.67 mmol) was added and dissolved with N,N-dimethylformamide (10 mL), and triethylamine (0.20 g, 2.02 mmol) and methylaminoformyl chloride (94 mg, 1.01 mmol) were added. After the addition was complete, the system was placed under nitrogen protection and stirred at 50° C. for 16 h. After the reaction was complete as monitored by TLC (dichloromethane:methanol=10:1), the reaction liquid was concentrated under reduced pressure, and the crude product was subjected to preparative HPLC to obtain the title compound 65 (200 mg, 66%).

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A with a content of 40-80%; c. flow: 15 mL/min; d. elution time: 20 min; retention time: 10.50 min.

LCMS m/z=504.2 [M+1]⁺

¹H NMR (400 MHZ, DMSO-d6) δ 8.89 (s, 2H), 7.60 (s, 1H), 7.29 (s, 1H), 6.95 (d, 1H), 4.46 (s, 2H), 4.43 (s, 2H), 3.97 (t, 2H), 3.40-3.48 (m, 1H), 3.18-3.25 (m, 1H), 2.85-2.96 (m, 2H), 2.65 (s, 2H), 2.56 (d, 3H), 2.20-2.45 (m, 3H), 1.90-1.80 (m, 3H).

### Example 66

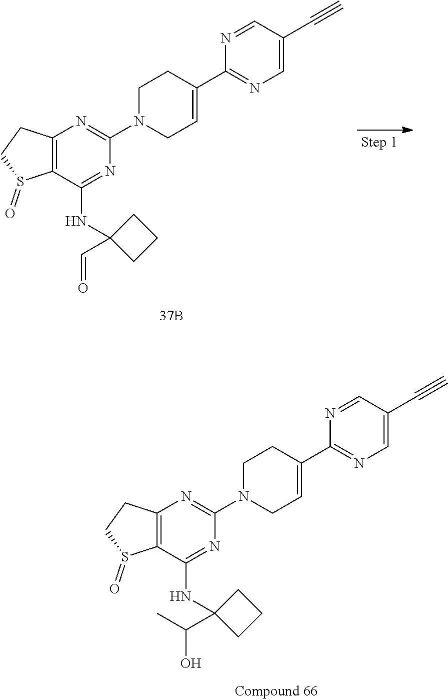 [SMILES: C#Cc1cnc(C2=CCN(c3nc4c(c(NC5(C=O)CCC5)n3)[S@](=O)CC4)CC2)nc1 ; C#Cc1cnc(C2=CCN(c3nc4c(c(NC5(C(C)O)CCC5)n3)[S@](=O)CC4)CC2)nc1]

Step 1: Compound 37B (200 mg, 0.46 mmol) was dissolved in anhydrous tetrahydrofuran (10 mL), and methylmagnesium bromide (0.46 mL, 1.38 mmol, 3.0 mol/L in diethyl ether) was dropwise added in an ice bath under nitrogen protection. After the addition was complete, the mixture was reacted in the ice bath for one hour. After quenching by saturated ammonium chloride, the system was extracted with ethyl acetate, the organic phase was dried, filtered, and concentrated, and the crude product was subjected to reverse phase preparation (acetonitrile:water=1/99-99/1) to obtain compound 66 (6 mg, 3%).

LC-MS (ESI): m/z=451.2 [M+H]⁺%

¹H NMR (400 MHz, DMSO-d₆) δ 8.90 (s, 2H), 7.38-7.29 (m, 2H), 4.92 (d, 1H), 4.69 (s, 1H), 4.53-4.41 (m, 2H), 4.30-4.16 (m, 1H), 4.07-3.97 (m, 1H), 3.97-3.87 (m, 1H), 3.48-3.37 (m, 1H), 3.27-3.22 (m, 1H), 3.00-2.91 (m, 1H), 2.90-2.82 (m, 1H), 2.69-2.62 (m, 2H), 2.43-2.32 (m, 3H), 2.28-2.21 (m, 1H), 1.84-1.69 (m, 2H), 1.00 (d, 3H).

### Example 67

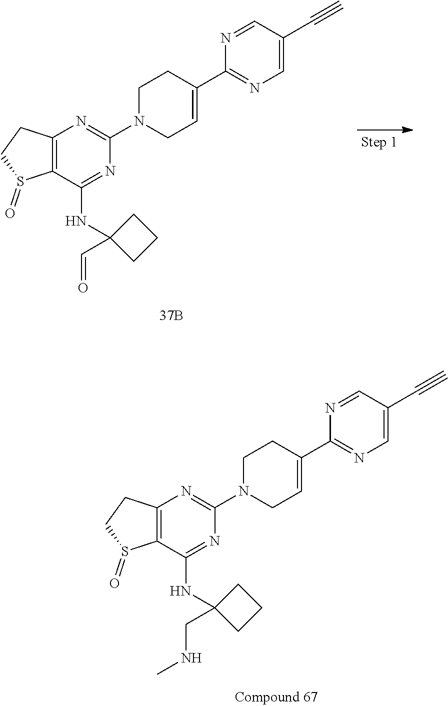 [SMILES: C#Cc1cnc(C2=CCN(c3nc4c(c(NC5(C=O)CCC5)n3)[S@](=O)CC4)CC2)nc1 ; C#Cc1cnc(C2=CCN(c3nc4c(c(NC5(CNC)CCC5)n3)[S@](=O)CC4)CC2)nc1]

Step 1: Compound 37B (300 mg, 0.69 mmol) was dissolved in 1,2-dichloroethane (10 mL) and acetic acid (10 mL), a methylamine solution (6.9 mL, 6.9 mmol, 3.0 mol/L in THF) was added in an ice bath, and after the addition was complete, the mixture was stirred at room temperature for 1 hour. Sodium triacetoxyborohydride (731 mg, 3.45 mmol) was then added and reacted at room temperature for 1 hour. After the reaction was complete, the system was quenched by saturated ammonium chloride and extracted with dichloromethane, the organic phase was dried, filtered, and concentrated, and the crude product was subjected to reverse phase preparation (acetonitrile:water=1/99-99/1) to obtain compound 67 (25 mg, 8%).

LC-MS (ESI): m/z=450.1 [M+H]⁺.

¹H NMR (400 MHz, DMSO-d₆) δ 8.91 (s, 2H), 8.43 (s, 1H), 7.65 (s, 1H), 7.42-7.28 (m, 1H), 4.69 (s, 1H), 4.60-4.35 (s, 2H), 4.04-3.88 (s, 2H), 3.64-3.40 (m, 3H), 3.23-3.14 (m, 1H), 3.05-2.85 (m, 2H), 2.68 (s, 2H), 2.56 (s, 3H), 2.47-2.25 (m, 4H), 1.93-1.79 (m, 2H).

### Example 68

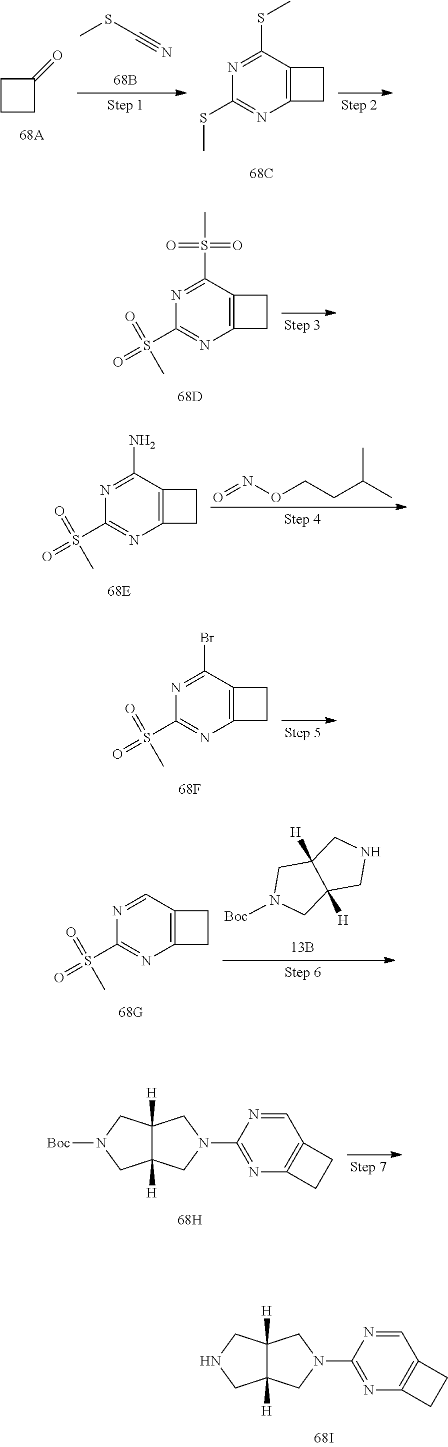

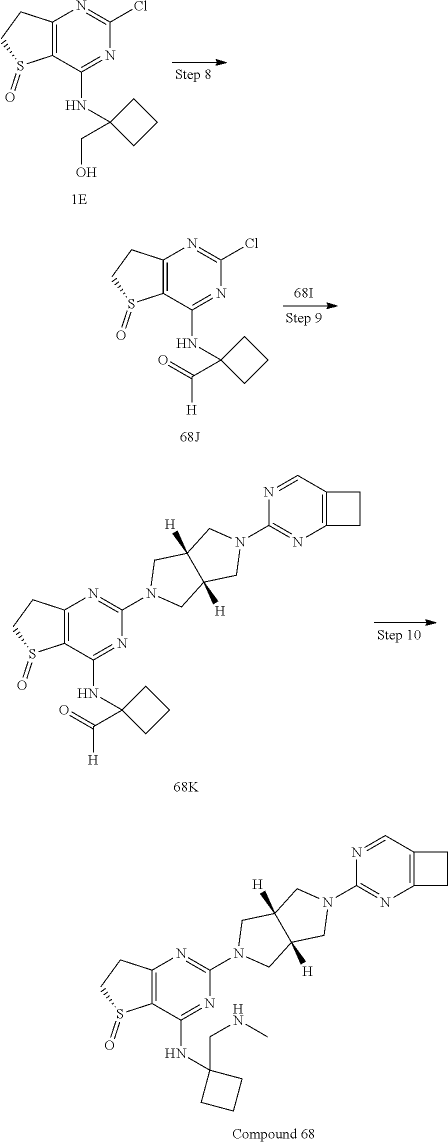 [SMILES: O=[S@@]1CCc2nc(Cl)nc(NC3(CO)CCC3)c21 ; O=CC1(Nc2nc(Cl)nc3c2[S@](=O)CC3)CCC1 ; O=CC1(Nc2nc(N3C[C@H]4CN(c5ncc6c(n5)CC6)C[C@H]4C3)nc3c2[S@](=O)CC3)CCC1 ; CNCC1(Nc2nc(N3C[C@H]4CN(c5ncc6c(n5)CC6)C[C@H]4C3)nc3c2[S@](=O)CC3)CCC1]

Step 1: To a 3 L three-mouth flask, methyl thiocyanate (68B, 132 g, 1.80 mol) was added and dissolved with 1,2-dichloroethane (1.20 L), trifluoromethanesulfonic anhydride (266 g, 0.940 mol) was added, and cyclobutanone (68A, 60.0 g, 0.860 mol) was slowly dropwise added at room temperature. After the addition was complete, the mixture was stirred at room temperature overnight. The reaction liquid was adjusted to pH 8-9 by adding a saturated sodium bicarbonate solution and left to stand for layering. An aqueous phase and an organic phase were separated. The organic phase was dried over anhydrous sodium sulfate, filtered, and concentrated, and the crude product was purified by silica gel column chromatography (dichloromethane/ethyl acetate (v/v)=99/1-95/5), followed by recrystallization (ethyl acetate/petroleum ether (v/v)=1/2) to obtain compound 68C (15.0 g, 9%).

LC-MS (ESI): m/z=199.1 [M+H]⁺

Step 2: To a 500 mL single-mouth flask, 68C (15.0 g, 75.6 mmol) was added and dissolved with dichloromethane (300 mL), and m-chloroperoxybenzoic acid (78.3 g, 454 mmol) was added and stirred at room temperature overnight. The reaction liquid was washed with a 10% sodium hydroxide aqueous solution (100 mL), and the organic phase was dried over anhydrous sodium sulfate, filtered, concentrated, and purified by column chromatography (petroleum ether/ethyl acetate (v/v)=50/50-0/100) to obtain compound 68D (3.8 g, 19%).

LC-MS (ESI): m/z=263.1 [M+H]⁺

Step 3: To a 100 mL single-mouth flask, 68D (3.8 g, 14.5 mmol) was added and dissolved with dichloromethane (40 mL), and aqueous ammonia (20 mL) was added and stirred at room temperature for 4 h. The reaction liquid was left to stand for direct phase separation. The aqueous phase was extracted once with dichloromethane (20 mL). The organic phases were combined, dried over anhydrous sodium sulfate, filtered, and concentrated, and the crude product was purified by column chromatography (dichloromethane/ethyl acetate (v/v)=67/33-0/100) to obtain the target compound 68E (2.2 g, 76%).

LC-MS (ESI): m/z=200.1 [M+H]⁺

Step 4: To a 100 mL single-mouth flask, 68E (2.2 g, 11.0 mmol) was added and dissolved with acetonitrile (40 ml), isoamyl nitrite (3.9 g, 33.1 mmol) and copper bromide (4.9 g, 22.1 mmol) were added, and the mixture was heated to 75° C. and stirred for 6 h under nitrogen protection. After cooling to room temperature, the reaction liquid was directly concentrated, and the crude product was added to water (100 mL) and extracted with dichloromethane (2×100 mL). The organic phases were combined, dried over anhydrous sodium sulfate, filtered, and concentrated, and the crude product was purified by column chromatography (petroleum ether/ethyl acetate (v/v)=75/25-50/50) to obtain the target compound 68F (2.0 g, 69%).

LC-MS (ESI): m/z=263.0 [M+H]⁺

Step 5: To a 100 mL single-mouth flask, 68F (2.0 g, 7.6 mmol) was added and dissolved with ethyl acetate (40 mL), palladium on carbon (10%, 6.0 g) was added, and the mixture was heated to 60° C. and stirred for 72 h. After cooling to room temperature, the reaction liquid was filtered through diatomite and washed clean with ethyl acetate, and the filtrate was concentrated and purified by column chromatography (dichloromethane/ethyl acetate (v/v)=67/33-0/100) to obtain the target compound 68G (0.48 g, 34%).

LC-MS (ESI): m/z=185.1 [M+H]⁺

Step 6: To a 50 mL single-mouth flask, 68G (0.18 g, 0.98 mmol) was added and dissolved with dioxane (8 mL), 13B (0.42 g, 1.96 mmol) and N,N-diisopropylethylamine (0.38 g, 2.94 mmol) were added, and the mixture was heated to 95° C. and stirred overnight. After cooling to room temperature, the reaction liquid was concentrated and purified by column chromatography (dichloromethane/ethyl acetate (v/v)=80/20-50/50) to obtain the target compound 68H (60 mg, 19%).

LC-MS (ESI): m/z=317.2 [M+H]⁺

Step 7: To a 50 mL single-mouth flask, 68H (60 mg, 0.19 mmol) was added and dissolved with dioxane (3 mL), and a hydrogen chloride-dioxane solution (3 mL, 4 N) was added and stirred at room temperature for 4 h. The reaction liquid was concentrated to obtain a crude product of the target compound 681 hydrochloride (48 mg), which was directly used in the next reaction.

LC-MS (ESI): m/z=217.2 [M+H]⁺

Step 8: Compound 35H (1.0 g, 3.48 mmol) was dissolved in ethyl acetate (15 ml), 2-iodoxybenzoic acid (2.93 g, 10.4 mmol) was added, and the mixture was stirred in an oil bath at 80° C. overnight. After the reaction was complete, the system was cooled to room temperature, filtered, concentrated, and quickly separated and purified by column chromatography (petroleum ether/ethyl acetate (v/v)=100/0-80/20) to obtain compound 68J (0.9 g, 82%).

LC-MS (ESI): m/z=286.1 [M+H]⁺

Step 9: To a 50 mL single-mouth flask, 68J (50 mg, 0.17 mmol) was added and dissolved with dioxane (6 mL), and crude 681 hydrochloride (44 mg, 0.20 mmol) and N,N-diisopropylethylamine (66 mg, 0.51 mmol) were successively added, heated to 95° C., and stirred overnight. After cooling to room temperature, the reaction liquid was concentrated and purified by column chromatography (dichloromethane/ethyl acetate (v/v)=95/5-90/10) to obtain the target compound 68K (70 mg, 88%).

LC-MS (ESI): m/z=466.1[M+H]⁺

Step 10: To a 50 mL single-mouth flask, 68K (50 mg, 0.11 mmol) was added and dissolved with tetrahydrofuran (5 mL) and methanol (2 mL), methylamine (0.5 mL, 2 N in THF) was added, and a drop of acetic acid was dripped. The mixture was stirred at room temperature for 1 h, and sodium triacetylborohydride (70 mg, 0.33 mmol) was added and stirred at room temperature overnight. The reaction liquid was concentrated and purified by HPLC to obtain the title compound 68 (3 mg, 6%).

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM ammonium acetate); b. gradient elution, mobile phase A with a content of 10-60%; c. flow: 15 mL/min; d. elution time: 20 min; retention time: 11.5 min.

¹H NMR (400 MHz, DMSO-d6) δ 7.90 (s, 1H), 7.30 (s, 1H), 3.80-3.70 (m, 6H), 3.43-3.35 (m, 4H), 3.25-3.19 (m, 4H), 3.22-2.94 (m, 5H), 2.92-2.80 (m, 2H), 2.45-2.41 (m, 2H), 2.31 (s, 3H), 2.15-2.09 (m, 2H), 1.84-1.68 (m, 2H).

LC-MS (ESI): m/z=481.5 [M+H]⁺

### Example 69

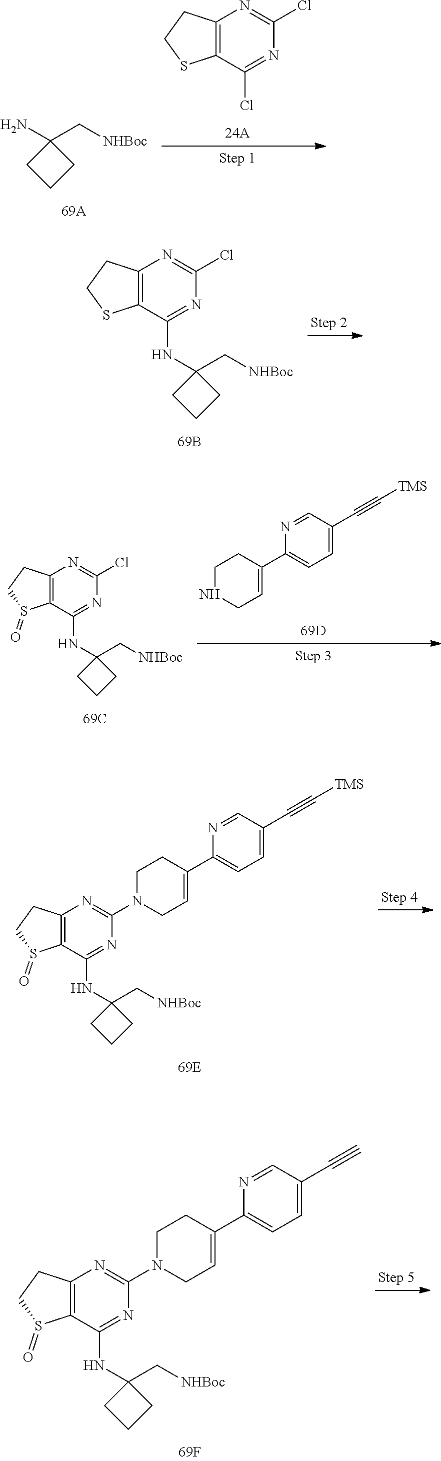

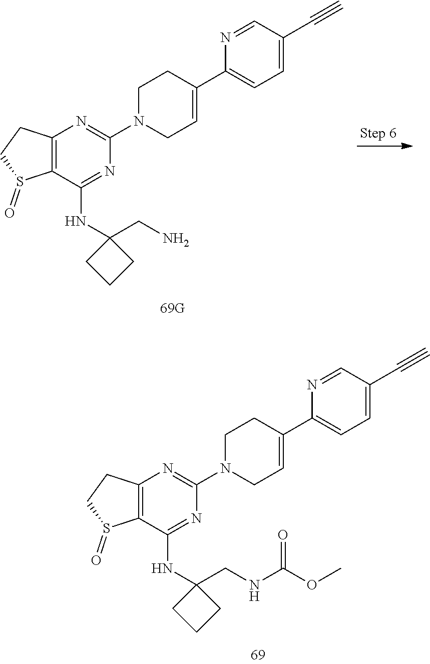 [SMILES: C#Cc1ccc(C2=CCN(c3nc4c(c(NC5(CN)CCC5)n3)[S@](=O)CC4)CC2)nc1 ; C#Cc1ccc(C2=CCN(c3nc4c(c(NC5(CNC(=O)OC)CCC5)n3)[S@](=O)CC4)CC2)nc1]

Step 1: Compound 69A (7.7 g, 38.6 mmol) was dissolved in acetonitrile (20 mL), compound 24A (8.0 g, 38.6 mmol) and triethylamine (19.5 g, 193.0 mmol) were then added, and the mixture was heated to 75° C., reacted for 12 h, then cooled to room temperature, concentrated, and subjected to column chromatography (dichloromethane/methanol (v/v)=93/7) to obtain compound 69B (5.5 g, 38%).

LC-MS (ESI) m/z=371.1 [M+H]⁺

Step 2: Compound 69B (2.7 g, 7.28 mmol), (S)-(−)-1,1′-bi-2-naphthol (0.21 g, 0.73 mmol), titanium tetraisopropoxide (0.1 g, 0.36 mmol), and water (0.13 g, 7.28 mmol) were dissolved in dichloromethane (20 mL). After nitrogen displacement three times, the system was stirred at room temperature for one hour, and tert-butyl hydroperoxide (0.72 mg, 8.01 mmol) was added and stirred for 1.5 hours. After quenching by adding a saturated sodium thiosulfate aqueous solution, the aqueous phase was extracted with dichloromethane, dried, concentrated, and subjected to column chromatography (dichloromethane/methanol (v/v)=90/10) to obtain compound 69C (3.0 g, 100%).

LC-MS (ESI): m/z=387.1 [M+H]⁺.

Step 3: Compound 69C (1.6 g, 4.14 mmol) was dissolved in 1,4-dioxane (15.0 mL), compound 69D (1.27 g, 4.97 mmol) and diisopropylethylamine (2.68 g, 20.7 mmol) were then added, and the mixture was heated to 90° C. and reacted for 12 h. The system was then cooled to room temperature, concentrated, and subjected to column chromatography (dichloromethane/methanol (v/v)=90/10) to obtain compound 69E (2.2 g, 87%).

LC-MS (ESI): m/z=607.1 [M+H]⁺.

Step 4: Compound 69E (2.2 g, 3.63 mmol) was dissolved in methanol (10 mL), and potassium carbonate (0.5 g, 3.63 mmol) was then added and reacted at room temperature for 2.5 hours. The system was then concentrated and subjected to column chromatography (dichloromethane/methanol (v/v)=90/10) to obtain compound 69F (1.9 g, 98%).

LC-MS (ESI): m/z=535.1 [M+H]⁺.

Step 5: Compound 69F (1.0 g, 1.87 mmol) was dissolved in dichloromethane (10 mL), and trifluoroacetic acid (3.0 mL) was then added and reacted at room temperature for 2.5 hours. The system was then concentrated and subjected to column chromatography (dichloromethane/methanol (v/v)=93/7) to obtain compound 69G (1.0 g, 98%).

LC-MS (ESI): m/z=435.1 [M+H]⁺.

Step 6: Compound 69G (1.0 g, 1.85 mmol) was dissolved in dichloromethane (10 mL), and triethylamine (0.37 g, 3.7 mmol) was added, followed by methyl chloroformate (0.188 g, 2.0 mmol). The mixture was reacted at room temperature for 1.5 hours. The system was concentrated and subjected to column chromatography (dichloromethane/methanol (v/v)=90/10) to obtain compound 69 (0.5 g, 55%).

¹H NMR (400 MHz, CDCl₃) δ 8.69 (d, 1H), 7.79-7.76 (m, 1H), 7.39 (d, 1H), 6.74 (s, 1H), 6.42 (s, 1H), 5.38 (s, 1H), 4.52 (s, 2H), 4.09 (s, 2H), 3.73-3.67 (m, 6H), 3.53-3.38 (m, 1H), 3.25 (s, 1H), 3.22-3.12 (m, 2H), 2.75 (s, 2H), 2.39 (s, 2H), 2.27-2.22 (m, 2H), 1.98-1.92 (m, 2H).

LC-MS (ESI): m/z=493.1 [M+H]⁺.

### Example 70

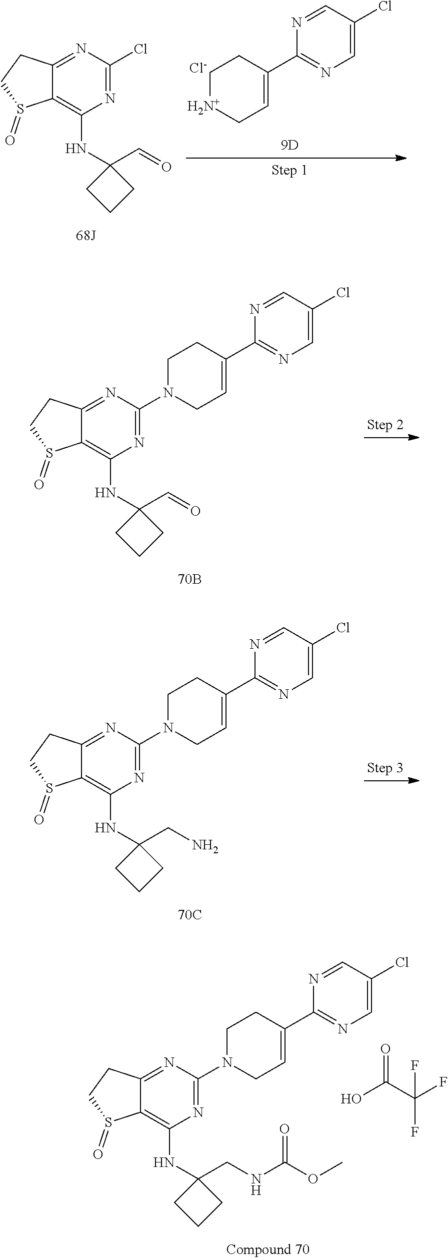

Step 1: Compound 68J (0.50 g, 1.75 mmol) and compound 9D (0.41 g, 2.10 mmol) were dissolved in 1,4-dioxane (20 mL), and N,N-diisopropylethylamine (0.90 g, 7.00 mmol) was added. After the addition was complete, the mixture was stirred at 90° C. for 16 h. After the reaction was complete as detected by TLC (dichloromethane:methanol=10:1), water (20 mL) was added and stirred for 5 min, and the mixture was extracted with ethyl acetate (20 mL). The organic phase was separated, dried over anhydrous sodium sulfate, and concentrated under reduced pressure, and the crude product was purified by column chromatography (dichloromethane/methanol (v/v)=90/10) to obtain the target compound 70B (0.5 g, 64%).

LC-MS (ESI): m/z=445.2 [M+H]⁺

Step 2: Compound 70B (0.16 g, 0.36 mmol) was weighed and dissolved with methanol (20 mL), and ammonium acetate (0.28 g, 3.63 mmol) was added. After the addition was complete, the mixture was stirred at room temperature for 12 h, sodium cyanoborohydride (34 mg, 0.54 mmol) was added, and the mixture was heated to 50° C. and stirred for 4 h. After the reaction was complete as detected by TLC (dichloromethane:methanol=5:1), the reaction liquid was directly concentrated under reduced pressure to obtain the target compound 70C (0.20 g, crude).

LC-MS (ESI): m/z=446.1 [M+H]⁺

Step 3: Crude compound 70C (200 mg) was dissolved in dichloromethane (10 mL), and triethylamine (0.14 g, 1.35 mmol) and methyl chloroformate (430 mg, 4.55 mmol) were added at 0° C. After the addition was complete, the system was placed under nitrogen protection and stirred at 10° C. for 4 h. After the reaction was complete as monitored by TLC (dichloromethane:methanol=5:1), the reaction liquid was concentrated under reduced pressure to obtain a crude product of the target compound, which was purified by preparative HPLC to obtain the title compound 70 (2.9 mg, 1.8%).

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM trifluoroacetic acid); b. gradient elution, mobile phase A with a content of 40-80%; c. flow: 15 mL/min; d. elution time: 20 min; retention time: 12.50 min.

¹H NMR (400 MHZ, DMSO-d6) δ 8.90 (s, 2H), 7.79 (s, 1H), 7.29 (s, 1H), 7.19 (t, 1H), 4.48 (s, 2H), 4.43 (s, 2H), 3.99 (t, 2H), 3.53-3.55 (m, 1H), 3.52 (s, 3H), 3.41-3.48 (m, 1H), 3.21-3.28 (m, 1H), 2.99-3.06 (m, 1H), 2.88-2.93 (m, 1H), 2.67 (s, 2H), 2.20-2.45 (m, 3H), 1.72-1.85 (m, 2H).

LC-MS (ESI): m/z=504.2 [M+H]⁺

### Example 71

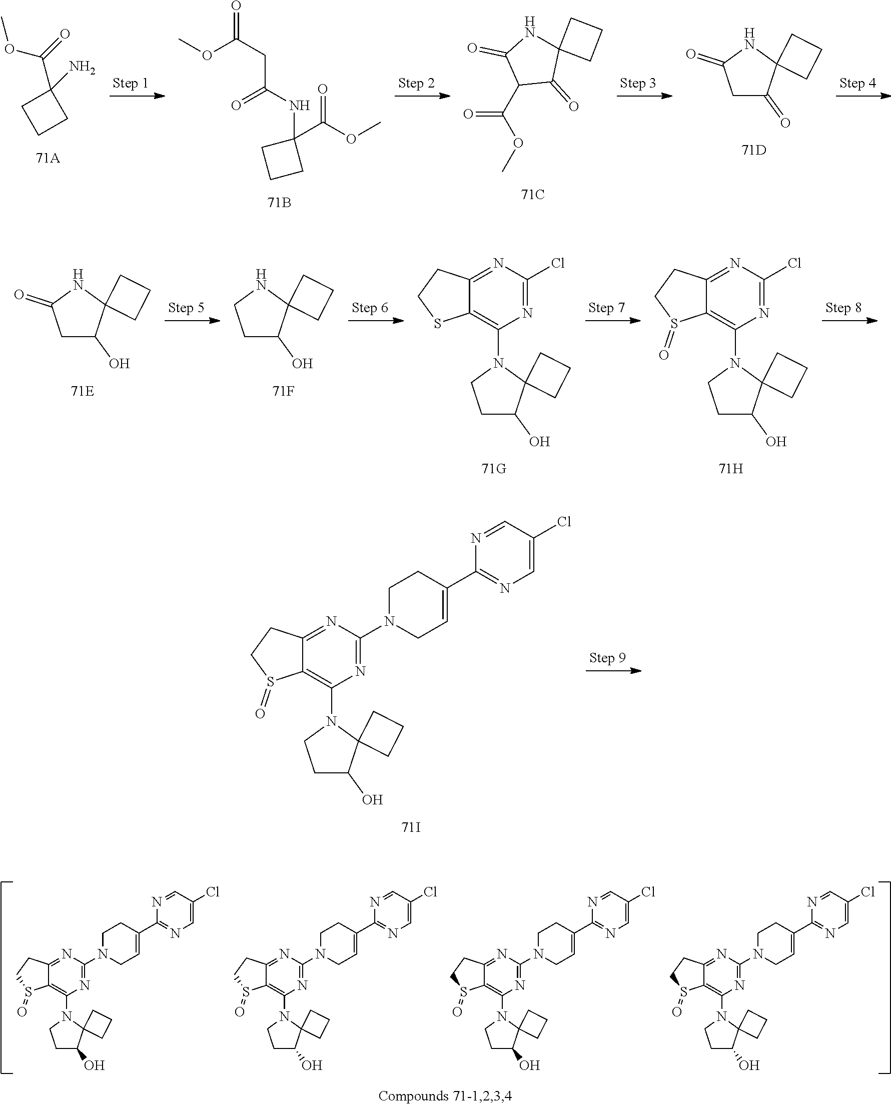

Step 1: 71A (10 g, 77.4 mmol) and triethylamine (9.4 g, 92.9 mmol) were dissolved in dichloromethane (100 mL), and methyl malonyl chloride (14.8 g, 108 mmol) was added in an ice bath and stirred in the ice bath for half an hour. The mixture was heated to room temperature, stirred for 3 hours, diluted by adding water, then extracted three times with dichloromethane, and washed with a saturated sodium bicarbonate solution. The organic phase was dried over anhydrous sodium sulfate. After filtration, the filtrate was concentrated to obtain crude 71B, which was directly used for the next reaction.

Step 2: The raw material 71B (15 g) was dissolved in anhydrous methanol (150 mL), sodium methoxide (7.78 g, 144 mmol) was added, and the mixture was heated to 65° C. and stirred for 10 hours. After cooling to room temperature, the system was directly filtered, and the filtrate was diluted by adding water, adjusted to pH 5-6 with 1N hydrochloric acid, extracted with dichloromethane, and dried over anhydrous sodium sulfate. After filtration, the filtrate was concentrated to obtain crude compound 71C, which was directly used for the next reaction.

Step 3: The raw material 71C (25 g) was dissolved in acetonitrile (250 mL), water (9 mL) was added, and the mixture was heated to 85° C. and stirred for 2 hours. The reaction liquid was cooled, concentrated, and then slurried with methyl tert-butyl ether to obtain compound 71D (6 g, three-step yield 34%).

¹H NMR (400 MHz, DMSO-d6) δ 8.76 (s, 1H), 2.99 (s, 2H), 2.15-2.19 (m, 2H), 1.79-1.82 (m, 2H), 1.66-1.77 (m, 2H).

LC-MS (ESI): m/z=140.1 [M+H]⁺.

Step 4: The raw material 71D (2.5 g, 17.97 mmol) was added to anhydrous methanol (50 mL), and sodium borohydride (1.36 g, 35.9 mmol) was then added in portions and stirred at room temperature for 10 hours. The reaction liquid was concentrated and then purified and separated by silica gel column chromatography (dichloromethane/methanol (v/v)=100/0-90/10) to obtain compound 71E (1.8 g, 71%).

LC-MS (ESI): m/z=142.1 [M+H]⁺.

Step 5: Lithium tetrahydroaluminate (1.45 g, 38.3 mmol) was suspended in anhydrous tetrahydrofuran (30 mL), and trimethylchlorosilane (3.46 g, 31.9 mmol) was added in an ice bath and stirred for half an hour. 71E (1.8 g, 12.8 mmol) was then added, and the mixture was heated to 65° C., stirred for 16 hours, and cooled. The reaction was quenched with a 30% potassium hydroxide aqueous solution, and the system was filtered. The filtrate was concentrated and then purified and separated by silica gel column chromatography (dichloromethane/methanol (v/v)=100/0-80/20) to obtain compound 71F (0.4 g, 25%).

LC-MS (ESI): m/z=128.1 [M+H]⁺

Step 6: Under nitrogen protection, 71F (0.45 g, 3.54 mmol), 24A (0.73 g, 3.54 mmol), and N,N-diisopropylethylamine (1.37 g, 10.6 mmol) were successively dissolved in acetonitrile (20 mL), heated to 45° C., stirred for 10 hours, cooled, then diluted by adding water, and extracted with ethyl acetate, and the organic phase was dried over anhydrous sodium sulfate. After filtration, the filtrate was concentrated and then purified and separated by silica gel column chromatography (petroleum ether/ethyl acetate (v/v)=100/0-50/50) to obtain compound 71G (0.4 g, 38%).

LC-MS (ESI): m/z=298.1 [M+H]⁺

Step 7: The raw material 71G (400 mg, 1.34 mmol), S-binaphthol (77 mg, 0.27 mmol), water (24 mg, 1.34 mmol), and tetraisopropyl titanate (38 mg, 0.13 mmol) were successively added to dichloromethane (15 mL) and stirred at room temperature for half an hour, and tert-butyl hydroperoxide (180 mg, 2.00 mmol) was added and further stirred for 3 hours. The mixture was diluted by adding water and extracted with dichloromethane, and the organic phase was washed with a saturated sodium bicarbonate aqueous solution and dried over anhydrous sodium sulfate. After filtration, the filtrate was concentrated and then purified and separated by a preparative silica gel plate (dichloromethane/methanol (v/v)=90/10) to obtain compound 71H (250 mg, 60%).

LC-MS (ESI): m/z=314.1 [M+H]⁺

Step 8: Under nitrogen protection, 71H (0.40 g, 1.27 mmol), intermediate 9D (0.25 g, 1.27 mmol), and N,N-diisopropylethylamine (0.82 g, 6.35 mmol) were successively dissolved in 1,4-dioxane (20 mL), heated to 95° C., stirred for 10 hours, cooled, diluted by adding water, and extracted with ethyl acetate, and the organic phases were combined and dried over anhydrous sodium sulfate. After filtration, the filtrate was concentrated and then purified and separated by silica gel column chromatography (dichloromethane/methanol (v/v)=100/0-90/10) to obtain two groups of isomers 71-1 and 2 and 71-3 and 4 of compound 71.

Compound 71-1 and compound 71-2 separation conditions: instrument name: Waters 150 Prep-SFC A; chromatographic column: Chiralcel OX column; mobile phase: A for CO₂ and B for isopropanol (0.1% NH₃·H₂O); gradient: 60%; flow rate: 100 mL/min; column pressure: 100 bar; column temperature: 25° C.; absorption wavelength: 220 nm; cycle time: about 8 min.

Compound 71-1, retention time: 1.592 min; ¹H NMR (400 MHz, DMSO-d6) δ 8.89 (s, 2H), 7.31-7.35 (m, 1H), 5.31 (d, 1H), 4.57-4.59 (m, 2H), 4.05-−4.17 (m, 3H), 3.86-3.92 (m, 1H), 3.70-3.74 (m, 1H), 3.42-3.54 (m, 2H), 3.32-3.35 (m, 1H), 3.14-3.21 (m, 1H), 3.04-3.10 (m, 1H), 2.91-2.96 (m, 1H), 2.70-2.72 (m, 2H), 2.26-2.30 (m, 1H), 1.87-1.99 (m, 3H), 1.65-1.79 (m, 2H).

LC-MS (ESI): m/z=473.40 [M+H]⁺

Compound 71-2, retention time: 2.034 min; ¹H NMR (400 MHz, DMSO-d6) δ 8.89 (s, 2H), 7.31-7.35 (m, 1H), 5.31 (d, 1H), 4.57-4.59 (m, 2H), 4.05-4.17 (m, 3H), 3.86-3.92 (m, 1H), 3.70-3.74 (m, 1H), 3.42-3.54 (m, 2H), 3.32-3.35 (m, 1H), 3.14-3.21 (m, 1H), 3.04-3.10 (m, 1H), 2.91-2.96 (m, 1H), 2.70-2.72 (m, 2H), 2.26-2.30 (m, 1H), 1.87-1.99 (m, 3H), 1.65-1.79 (m, 2H).

LC-MS (ESI): m/z=473.40 [M+H]⁺

Compound 71-3 and compound 71-4 separation conditions: instrument name: Waters 150 Prep-SFC A; chromatographic column: Chiralcel Whelk column; mobile phase: A for CO₂ and B for 0.1% NH₃·H₂O in ETOH and ACN; gradient: 55%; flow rate: 100 mL/min; column pressure: 100 bar; column temperature: 25° C.; absorption wavelength: 220 nm; cycle time: about 7.5 min.

Compound 71-3, retention time: 1.577 min; ¹H NMR (400 MHz, DMSO-d6) δ 8.89 (s, 2H), 7.31-7.35 (m, 1H), 5.32 (d, 1H), 4.56-4.58 (m, 2H), 4.06-4.13 (m, 3H), 3.86-3.91 (m, 2H), 3.41-3.56 (m, 2H), 3.29-3.37 (m, 1H), 3.11-3.17 (m, 1H), 2.96-3.03 (m, 2H), 2.70-2.72 (m, 2H), 2.24-2.32 (m, 1H), 1.93-1.99 (m, 1H), 1.80-1.90 (m, 2H), 1.70-1.79 (m, 2H).

LC-MS (ESI): m/z=473.40 [M+H]⁺

Compound 71-4, retention time: 2.246 min; ¹H NMR (400 MHz, DMSO-d6) δ 8.89 (s, 2H), 7.31-7.35 (m, 1H), 5.32 (d, 1H), 4.56-4.58 (m, 2H), 4.06-4.13 (m, 3H), 3.86-3.91 (m, 2H), 3.41-3.56 (m, 2H), 3.29-3.37 (m, 1H), 3.11-3.17 (m, 1H), 2.96-3.03 (m, 2H), 2.70-2.72 (m, 2H), 2.24-2.32 (m, 1H), 1.93-1.99 (m, 1H), 1.80-1.90 (m, 2H), 1.70-1.79 (m, 2H).

LC-MS (ESI): m/z=473.40 [M+H]⁺

### Example 72

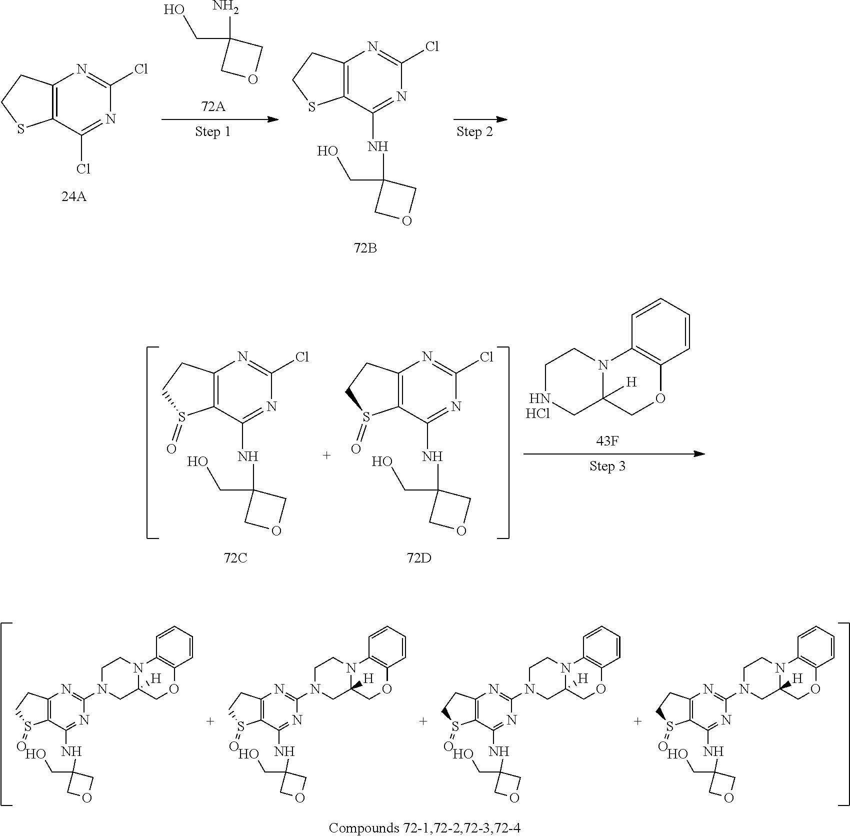

Using compounds 72A and 24A as raw materials, a mixture of compounds 72-1 and 72-2 (0.25 g, 84%) was obtained according to the operation of Example 34.

Chiral preparation was carried out to obtain the title compound 72-1 (20 mg, retention time: 1.535 min) and the title compound 72-2 (30 mg, retention time: 2.012 min).

Chiral preparation method: instrument: Waters 150 preparative SFC (SFC-26); chromatographic column: ChiralPak AD, 250×30 mm I.D., 10 μm; mobile phase: A: carbon dioxide, and B: isopropanol (0.1% aqueous ammonia); isocratic elution: 30% mobile phase B; flow rate: 150 mL/min; back pressure: 100 bar; column temperature: 38° C.; wavelength: 220 nm; elution time: 15 min.

According to the above operation, using 72D (195 mg) as a raw material, a mixture of 72-3 and 72-4 (0.26 g, 87%) was obtained.

Chiral preparation was carried out to obtain the title compound 72-3 (50 mg, retention time: 0.698 min) and the title compound 72-4 (50 mg, retention time: 1.514 min).

Chiral preparation method: instrument: Waters 150 preparative SFC (SFC-26); chromatographic column: ChiralPak AD, 250×30 mm I.D., 10 μm; mobile phase: A: carbon dioxide, and B: ethanol (0.1% aqueous ammonia); isocratic elution: 65% mobile phase B; flow rate: 100 mL/min; back pressure: 100 bar; column temperature: 38° C.; wavelength: 220 nm; elution time: 5 min.

### (Compound 72-1)

¹H NMR (400 MHz, DMSO-d6) δ 8.21 (s, 1H), 6.94-6.88 (m, 1H), 6.84-6.74 (m, 1H), 6.73-6.62 (m, 2H), 5.18-5.12 (m, 1H), 4.74-4.57 (m, 6H), 4.39-4.32 (m, 1H), 3.99-3.92 (m, 1H), 3.88-3.77 (m, 3H), 3.49-3.40 (m, 1H), 3.26-3.18 (m, 1H), 3.11-2.86 (m, 4H), 2.76-2.65 (m, 1H), 2.63-2.54 (m, 1H).

LC-MS (ESI): m/z=444.2 [M+H]⁺.

### (Compound 72-2)

¹H NMR (400 MHz, DMSO-d6) δ 8.21 (s, 1H), 6.94-6.88 (m, 1H), 6.83-6.76 (m, 1H), 6.73-6.62 (m, 2H), 5.18-5.12 (m, 1H), 4.74-4.57 (m, 6H), 4.39-4.32 (m, 1H), 3.99-3.92 (m, 1H), 3.88-3.76 (m, 3H), 3.49-3.40 (m, 1H), 3.26-3.19 (m, 1H), 3.12-2.86 (m, 4H), 2.76-2.66 (m, 1H), 2.64-2.54 (m, 1H).

LC-MS (ESI): m/z=444.2 [M+H]⁺.

### (Compound 72-3)

¹H NMR (400 MHz, DMSO-d6) δ 8.21 (s, 1H), 6.94-6.88 (m, 1H), 6.83-6.77 (m, 1H), 6.73-6.62 (m, 2H), 5.19-5.12 (m, 1H), 4.74-4.57 (m, 6H), 4.39-4.32 (m, 1H), 3.99-3.91 (m, 1H), 3.87-3.76 (m, 3H), 3.49-3.41 (m, 1H), 3.28-3.18 (m, 1H), 3.12-2.86 (m, 4H), 2.76-2.66 (m, 1H), 2.64-2.55 (m, 1H).

LC-MS (ESI): m/z=444.2 [M+H]⁺.

### (Compound 72-4)

¹H NMR (400 MHz, DMSO-d6) δ 8.21 (s, 1H), 6.94-6.88 (m, 1H), 6.82-6.78 (m, 1H), 6.72-6.63 (m, 2H), 5.19-5.12 (m, 1H), 4.74-4.57 (m, 6H), 4.39-4.32 (m, 1H), 3.99-3.91 (m, 1H), 3.87-3.76 (m, 3H), 3.49-3.41 (m, 1H), 3.28-3.18 (m, 1H), 3.12-2.86 (m, 4H), 2.76-2.66 (m, 1H), 2.64-2.55 (m, 1H).

LC-MS (ESI): m/z=444.2 [M+H]⁺.

### Example 73

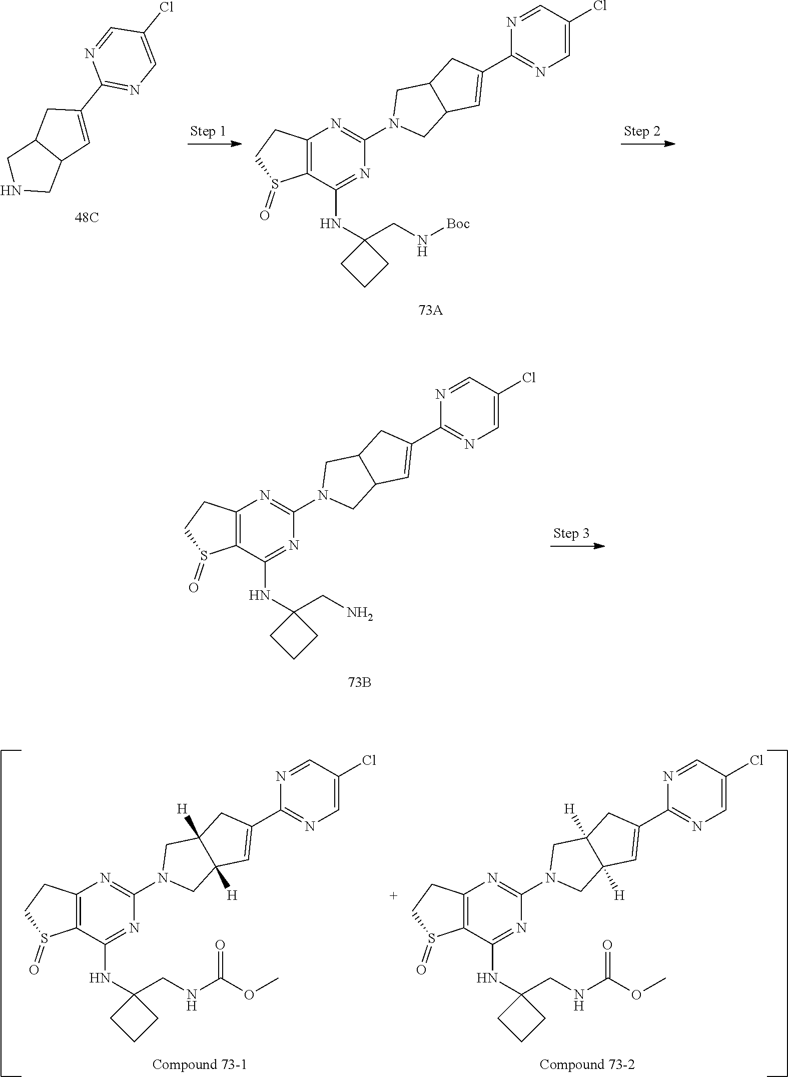

Using compounds 46C and 69C as raw materials, compound 73-1 (130 mg, retention time: 2.192 min) and the title compound 73-2 (138.2 mg, retention time: 2.573 mi) were obtained according to the operation of Example 69.

Chiral preparation method: instrument: Waters 150 preparative SFC (SFC-26); chromatographic column: Chiralcel Cellulose-2 column; mobile phase: A: carbon dioxide, and B: methanol and acetonitrile (0.1% aqueous ammonia); isocratic elution: 40% mobile phase B; flow rate: 100 mL/min; back pressure: 100 bar; column temperature: 25° C.; wavelength: 220 nm; elution time: 7.0 min.

### (Compound 73-1)

¹H NMR (400 MHz, DMSO-d6) δ 8.87 (s, 2H), 7.36 (s, 1H), 7.13 (s, 1H), 6.85 (s, 1H), 3.94-3.84 (m, 1H), 3.78 (s, 1H), 3.66 (s, 2H), 3.51 (s, 3H), 3.45-3.33 (m, 2H), 3.24-3.10 (m, 3H), 2.86-2.81 (m, 1H), 2.75 (d, 1H), 2.37-2.13 (m, 4H), 1.76 (s, 2H), 1.39 (d, 1H), 1.17 (d, 2H).

LCMS (ESI): m/z=530.2 [M+H]⁺

### Compound 73-2:

¹H NMR (400 MHz, DMSO-d6) δ 8.87 (s, 2H), 7.35 (s, 1H), 7.11 (s, 1H), 6.85 (s, 1H), 3.80-3.76 (m, 1H), 3.77 (s, 1H), 3.66 (s, 2H), 3.50 (s, 3H), 3.40 (s, 1H), 3.25-3.12 (m, 3H), 3.04-2.79 (m, 4H), 2.31-2.13 (m, 3H), 1.77 (s, 2H), 1.39 (d, 1H), 1.17 (d, 2H).

LCMS (ESI): m/z=530.2 [M+H]⁺

### Example 74

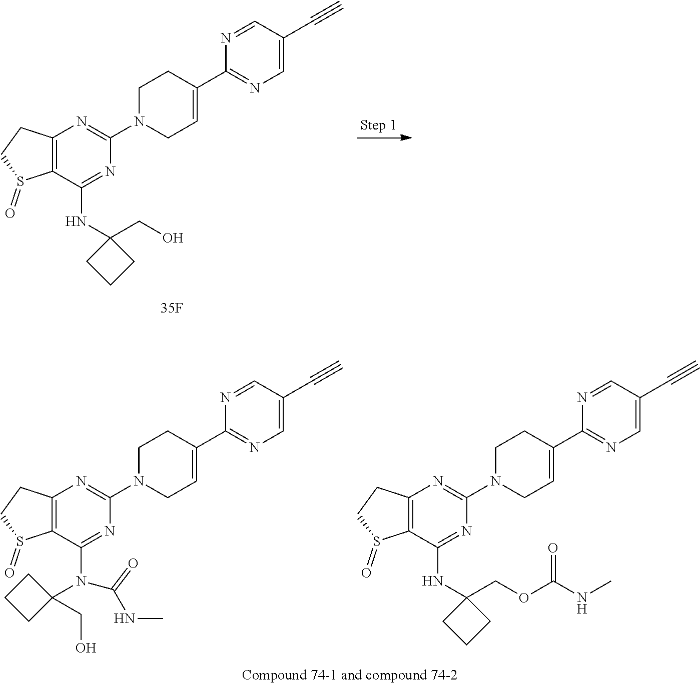 [SMILES: C#Cc1cnc(C2=CCN(c3nc4c(c(NC5(CO)CCC5)n3)[S@](=O)CC4)CC2)nc1 ; C#Cc1cnc(C2=CCN(c3nc4c(c(N(C(=O)NC)C5(CO)CCC5)n3)[S@](=O)CC4)CC2)nc1 ; C#Cc1cnc(C2=CCN(c3nc4c(c(NC5(COC(=O)NC)CCC5)n3)[S@](=O)CC4)CC2)nc1]

Step 1: Compound 35F (0.85 g, 2.0 mmol) was dissolved in tetrahydrofuran (10 mL), and sodium hydride (80.0 mg, 2.0 mmol) was added. After the addition was complete, the mixture was reacted at room temperature for 1 hour, and methylaminoformyl chloride (186.0 mg, 2.0 mmol) was further added and further reacted at room temperature overnight. After the reaction was complete as monitored by TLC, the reaction was quenched by adding a small amount of methanol. The reaction liquid was concentrated, and the crude product was purified by silica gel column chromatography (dichloromethane/methanol (v/v)=95/5) to obtain compound 74-1 (0.11 g, 11%) and 74-2 (0.25 g, 23%).

LC-MS (ESI): m/z=494.1 [M+H]⁺¹H NMR (400 MHz, DMSO-d6) δ 8.90 (s, 2H), 7.58 (s, 1H), 7.34 (s, 1H), 6.93 (s, 1H), 4.68 (s, 1H), 4.45 (d, 3H), 3.97 (s, 2H), 3.52-3.36 (m, 1H), 3.23 (d, 2H), 2.92 (m, 1H), 2.67 (s, 3H), 2.56 (d, 3H), 2.35 (d, 2H), 2.25 (s, 2H), 1.82-1.85 (m, 2H).

LC-MS (ESI): m/z=494.1 [M+H]⁺.

¹H NMR (400 MHz, DMSO-d6) δ 8.90 (s, 2H), 7.80 (s, 1H), 7.54 (s, 1H), 6.92 (d, 1H), 5.18-5.15 (m, 1H), 4.64 (s, 1H), 4.39 (s, 2H), 4.11 (s, 1H), 3.83 (d, 2H), 3.56-3.40 (m, 1H), 3.28-3.15 (m, 1H), 3.03-2.88 (m, 2H), 2.55 (d, 3H), 2.40-2.31 (m, 2H), 2.23 (s, 4H), 1.90-1.75 (m, 2H).

### Example 75

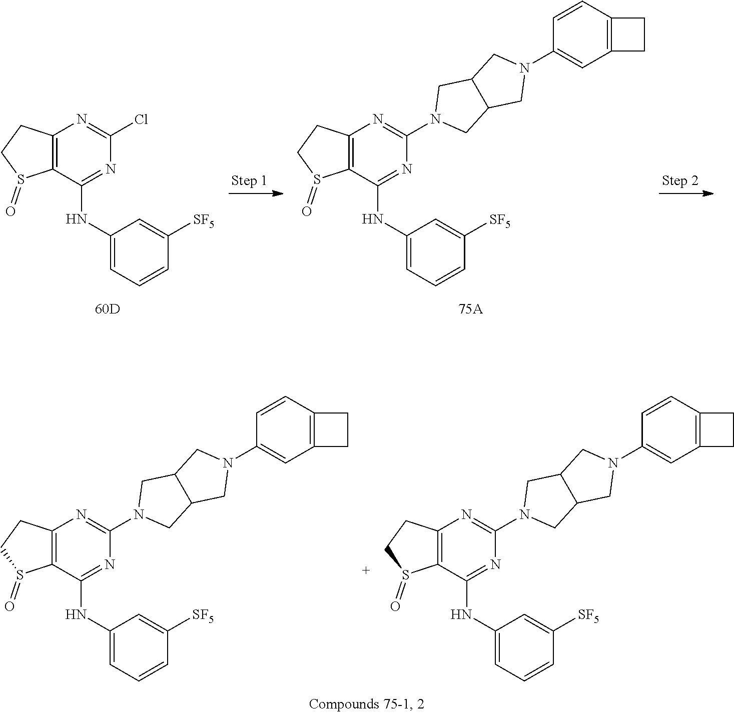

Using compounds 60D and 13D as raw materials, 75-1 (71.1 mg) and compound 75-2 (36.8 mg) were obtained according to the operation of Example 60.

Analysis method: instrument: SHIMADZU LC-20AD, column: Chiralpak IG-3 50×4.6 mm I.D., 3 μm; mobile phase: A: water (0.1% TFA), B: acetonitrile; gradient: 10-80% B in A; flow rate: 1.2 mL/min, column temperature: 45° C., wavelength: 210 & 254 nm.

Preparation method: instrument: SHIMADZU LC-20AP, column: DAICEL CHIRALPAK IG (250 mm*30 mm, 10 μm) mobile phase: A: water (0.01% NH4HCO3 in H2O), B: acetonitrile; gradient: 70-100% B in A; flow rate: 25 mL/min, column temperature: 25° C., wavelength: 254 nm, cycle time: 16 min, sample preparation: sample concentration 1.5 mg/ml, ethanol solution injection: 2 ml each time. After separation, the fraction was dried by a rotary evaporator at a bath temperature of 40° C. to obtain compound 75-1 (retention time: 1.340 min) and compound 75-2 (retention time: 1.935 min).

### (Compound 75-1)

¹H NMR (400 MHz, CDCl₃) δ 8.79-8.50 (m, 2H), 7.49-7.47 (m, 1H), 7.37-7.36 (m, 1H), 7.31-7.29 (m, 1H), 6.93-6.87 (m, 1H), 6.44-6.33 (m, 2H), 3.89-3.81 (m, 2H), 3.66-3.33 (m, 6H), 3.25-3.07 (m, 10H).

LCMS m/z=584.1 [M+H]⁺

### (Compound 75-2)

¹H NMR (400 MHz, CDCl₃) δ 8.80-8.50 (m, 2H), 7.49-7.47 (m, 1H), 7.37-7.36 (m, 1H), 7.31-7.29 (m, 1H), 6.93-6.87 (m, 1H), 6.44-6.33 (m, 2H), 3.89-3.81 (m, 2H), 3.66-3.19 (m, 6H), 3.25-3.07 (m, 10H).

LCMS m/z=584.1 [M+H]⁺

### Example 76

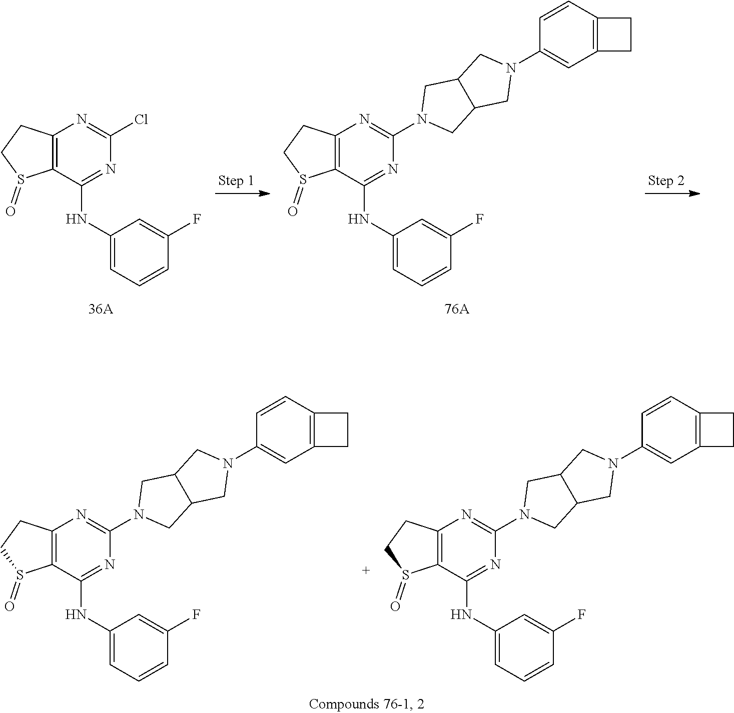 [SMILES: O=S1CCc2nc(Cl)nc(Nc3cccc(F)c3)c21 ; O=S1CCc2nc(N3CC4CN(c5ccc6c(c5)CC6)CC4C3)nc(Nc3cccc(F)c3)c21 ; O=[S@@]1CCc2nc(N3CC4CN(c5ccc6c(c5)CC6)CC4C3)nc(Nc3cccc(F)c3)c21 ; O=[S@]1CCc2nc(N3CC4CN(c5ccc6c(c5)CC6)CC4C3)nc(Nc3cccc(F)c3)c21]

Using compounds 36A and 13D as raw materials, compound 76-1 (342 mg) and compound 76-2 (50 mg) were obtained according to the operation of Example 60. Analysis method: instrument: SHIMADZU LC-20AD, column: Chiralpak IG-3 50×4.6 mm I.D., 3 μm; mobile phase: A: water (0.1% TFA), B: acetonitrile; gradient: 10-80% B in A; flow rate: 1.2 mL/min, column temperature: 45° C., wavelength: 210 & 254 nm.

Preparation method: instrument: SHIMADZU LC-20AP, column: DAICEL CHIRALPAK IG (250 mm×30 mm, 10 μm) mobile phase: A: water (0.01% NH₄HCO₃ in H₂O), B: acetonitrile; gradient: 60% B in A; flow rate: 25 mL/min, column temperature: 25° C., wavelength: 254 nm, cycle time: 7 min, sample preparation: sample concentration 1.5 mg/mL, ethanol solution injection: 2 mL each time. After separation, the fraction was dried by a rotary evaporator at a bath temperature of 40° C. to obtain compound 76-1 (retention time: 1.542 min) and compound 76-2 (retention time: 2.226 min).

### (Compound 76-1)

¹H NMR (400 MHz, CDCl₃) δ 8.30-8.21 (m, 1H), 7.82-7.76 (m, 1H), 7.21-7.14 (m, 2H), 6.95-6.87 (m, 1H), 6.72-6.68 (m, 1H), 6.47-6.33 (m, 2H), 3.91-3.83 (m, 2H), 3.64-3.55 (m, 5H), 3.39-3.20 (m, 3H), 3.16-3.02 (m, 8H).

LCMS m/z=476.1 [M+H]⁺

### (Compound 76-2)

¹H NMR (400 MHz, CDCl₃) δ 8.30-8.21 (m, 1H), 7.82-7.76 (m, 1H), 7.21-7.14 (m, 2H), 6.95-6.87 (m, 1H), 6.72-6.68 (m, 1H), 6.47-6.33 (m, 2H), 3.91-3.83 (m, 2H), 3.64-3.52 (m, 5H), 3.39-3.20 (m, 3H), 3.16-3.02 (m, 8H).

LCMS m/z=476.1 [M+H]⁺

### Example 77

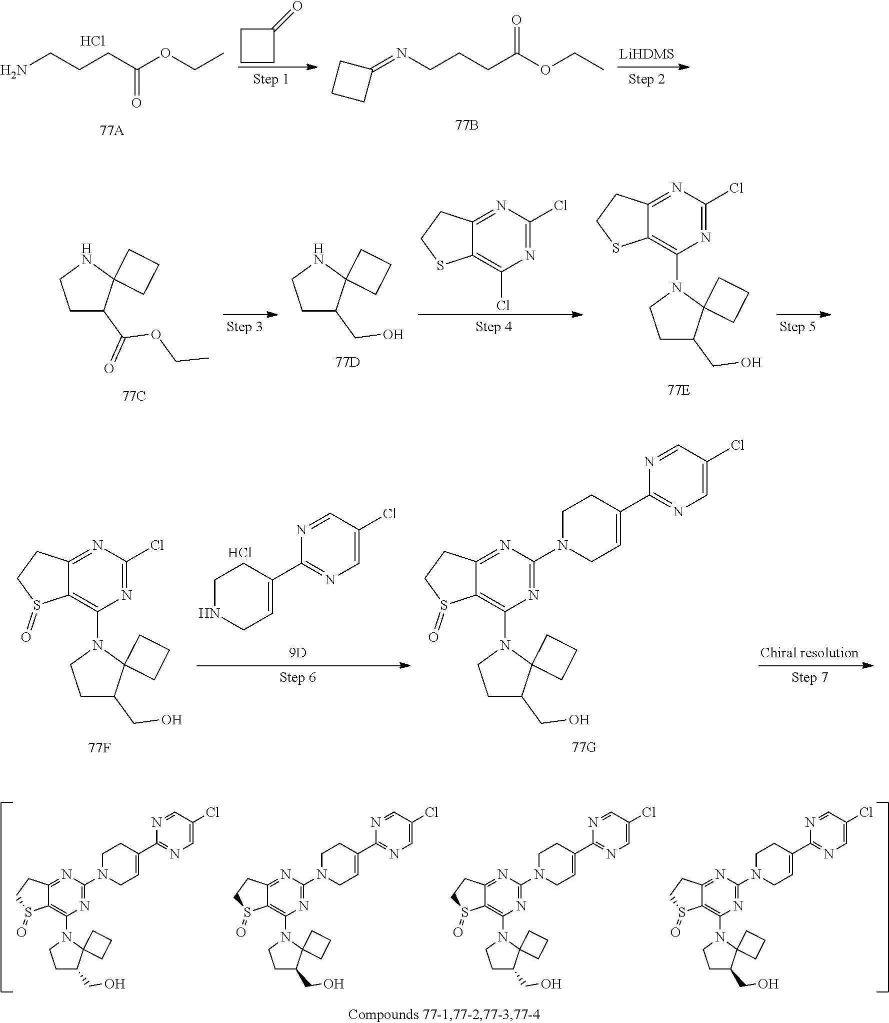

Step 1: Compound 77A (11.5 g, 68 mmol), cyclobutanone (4.8 g, 68 mmol), and potassium carbonate (28.1 g, 204 mmol) were weighed into a three-mouth flask, dissolved by adding toluene (200 mL), and reacted at 60° C. for 24 h. After the reaction was complete as monitored by LCMS, the system was cooled to room temperature and filtered. The filter cake was washed with ethyl acetate (50 mL), and the filtrate was concentrated to obtain a crude product of the target compound 77B (15.8 g), which did not require further purification.

Step 2: Crude compound 77B (15.8 g) was weighed into a three-mouth flask and dissolved by adding anhydrous tetrahydrofuran (200 mL). After nitrogen displacement, the mixture was cooled to −78° C., and a lithium bis(trimethylsilyl)amide solution (70 mL, 1M in THF) was slowly dropwise added. After the dropwise addition was complete, the mixture was naturally heated to room temperature and adjusted to weak acidity with a saturated ammonium chloride solution. The organic phase was separated, and the aqueous phase was extracted with ethyl acetate (100 mL×1). The organic phases were combined, dried over anhydrous sodium sulfate, and filtered. After concentration, the residue was separated by column chromatography (petroleum ether/ethyl acetate (v/v)=100/0-83/17) to obtain the title compound 77C (4.9 g, 39%).

LC-MS (ESI): m/z=184.2 [M+H]⁺.

Step 3: 77C (4.9 g, 26.7 mmol) was dissolved in tetrahydrofuran (50 mL), and lithium tetrahydroaluminate (1.9 g, 52 mmol) was slowly added. After the raw material completely disappeared as monitored by TLC, water (2 mL) was slowly added first, followed by a 15% sodium hydroxide aqueous solution (2 mL), and the mixture was fully stirred and then filtered. The filtrate was concentrated to obtain the title compound 77D (1.23 g, 32.6%).

LC-MS (ESI): m/z=142.2 [M+H]⁺.

Step 4: Compound 77D (1 g, 7 mmol) was dissolved in anhydrous acetonitrile, and 2,4-dichloro-6H,7H-thieno[3,2-d]pyrimidine (1.45 g, 7 mmol) and triethylamine (1.4 g, 14 mmol) were added. The mixture was heated to 55° C. and reacted overnight, and the system was cooled to room temperature and then directly concentrated. The residue was separated by column chromatography (petroleum ether/ethyl acetate (v/v)=100/0-50/50) to obtain the title compound 77E (668 mg, 30.3%).

LC-MS (ESI): m/z=312.2 [M+H]⁺.

Step 5: Compound 77E (668 mg, 2.14 mmol) was dissolved in dichloromethane, TIPT (1.21 g, 4.28 mmol) and tert-butyl hydroperoxide (231 mg, 2.56 mmol) were successively added, and the mixture was reacted at room temperature for 4 h. The system was concentrated, and the residue was separated by silica gel column chromatography (petroleum ether/ethyl acetate (v/v)=100/0-0/100) to obtain the title compound 77F (551 mg, 78.5%).

LC-MS (ESI): m/z=328.2 [M+H]⁺.

Step 6: Compound 77F (551 mg, 1.68 mmol) was dissolved in 1,4-dioxane, and 9D (268 mg, 1.68 mmol) and N,N-diisopropylethylamine (650 mg, 5.04 mmol) were successively added and reacted at 85° C. for 4 h. They system was cooled to room temperature and then directly concentrated, and the residue was purified and separated by silica gel column chromatography (dichloromethane/methanol (v/v)=91/9) to obtain two crude compounds 77G-1 (142 mg) and 77G-2 (136 mg).

LC-MS (ESI): m/z=487.7 [M+H]⁺

Step 7: 77G-1 was purified and separated by chiral HPLC to obtain compound 77-1 (44 mg, 1.126 min) and 77-2 (32 mg, 1.586 min). Separation conditions: instrument name: Waters 150 Prep-SFC F; chromatographic column: Chiralcel AS column; mobile phase: A for CO₂; B for 0.1% NH₃·H₂O in IPA and ACN; gradient: 45%; flow rate: 100 mL/min; column pressure: 100 bar; column temperature: 25° C.; absorption wavelength: 220 nm; cycle time: about 5 min).

### (Compound 77-1)

¹H NMR (400 MHz, DMSO-d₆) δ 8.89 (s, 2H), 7.35-7.29 (m, 1H), 4.72 (s, 1H), 4.61-4.51 (m, 2H), 4.10-4.03 (m, 2H), 3.94-3.78 (m, 2H), 3.75-3.64 (m, 1H), 3.62-3.38 (m, 4H), 3.20-3.10 (m, 1H), 3.10-2.91 (m, 2H), 2.70 (s, 2H), 2.34-2.22 (m, 1H), 2.14-1.69 (m, 6H).

LC-MS (ESI): m/z=487.7 [M+H]⁺

### (Compound 77-2)

¹H NMR (400 MHz, DMSO-d₆) δ 8.89 (s, 2H), 7.35-7.29 (m, 1H), 4.72 (s, 1H), 4.61-4.51 (m, 2H), 4.10-4.03 (m, 2H), 3.94-3.78 (m, 2H), 3.75-3.64 (m, 1H), 3.62-3.38 (m, 4H), 3.20-3.10 (m, 1H), 3.10-2.91 (m, 2H), 2.70 (s, 2H), 2.34-2.22 (m, 1H), 2.14-1.69 (m, 6H).

LC-MS (ESI): m/z=487.7 [M+H]⁺

77G-2 was purified and separated by chiral HPLC to obtain compound 77-3 (31 mg, 1.300 min) and 77-4 (35 mg, 2.304 min). Separation conditions: instrument name: Waters 150 Prep-SFC F; chromatographic column: Chiralcel AS column; mobile phase: A for CO₂; B for 0.1% NH₃·H₂O in IPA and ACN; gradient: 55%; flow rate: 100 mL/min; column pressure: 100 bar; column temperature: 25° C.; absorption wavelength: 220 nm; cycle time: about 6.3 min.

### (Compound 77-3)

¹H NMR (400 MHz, DMSO-d₆) δ 8.89 (s, 2H), 7.35-7.27 (m, 1H), 4.73-4.68 (m, 1H), 4.59-4.54 (m, 2H), 4.13-4.01 (m, 2H), 3.92-3.85 (m, 1H), 3.82-3.71 (m, 2H), 3.70-3.54 (m, 2H), 3.54-3.42 (m, 1H), 3.41-3.32 (m, 1H), 3.22-3.11 (m, 1H), 3.09-3.00 (m, 1H), 3.00-2.90 (m, 1H), 2.70 (s, 2H), 2.23-2.15 (m, 1H), 2.10-2.00 (m, 2H), 1.98-1.86 (m, 2H), 1.84-1.66 (m, 2H).

LC-MS (ESI): m/z=487.7 [M+H]⁺

### (Compound 77-4)

¹H NMR (400 MHz, DMSO-d₆) δ 8.89 (s, 2H), 7.35-7.27 (m, 1H), 4.73-4.68 (m, 1H), 4.59-4.54 (m, 2H), 4.13-4.01 (m, 2H), 3.92-3.85 (m, 1H), 3.82-3.71 (m, 2H), 3.70-3.54 (m, 2H), 3.54-3.42 (m, 1H), 3.41-3.32 (m, 1H), 3.22-3.11 (m, 1H), 3.09-3.00 (m, 1H), 3.00-2.90 (m, 1H), 2.70 (s, 2H), 2.23-2.15 (m, 1H), 2.10-2.00 (m, 2H), 1.98-1.86 (m, 2H), 1.84-1.66 (m, 2H).

LC-MS (ESI): m/z=487.7 [M+H]⁺

### Example 78

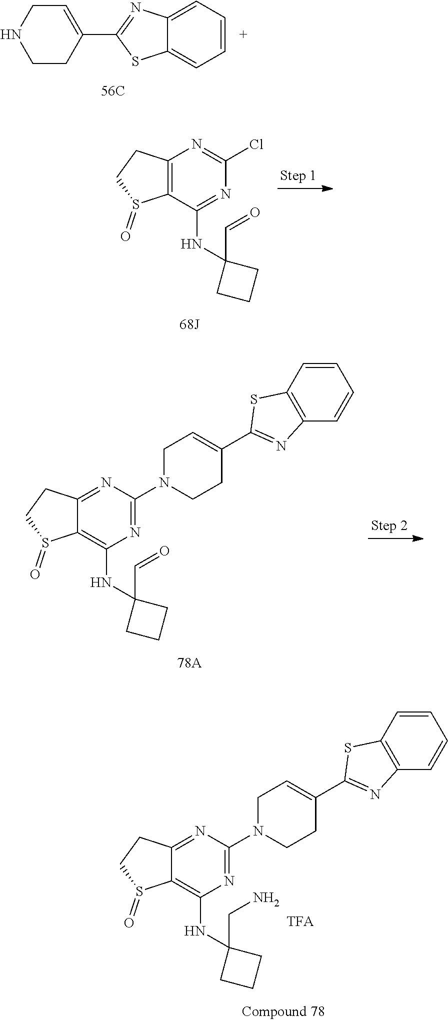 [SMILES: C1=C(c2nc3ccccc3s2)CCNC1 ; O=CC1(Nc2nc(Cl)nc3c2[S@](=O)CC3)CCC1 ; O=CC1(Nc2nc(N3CC=C(c4nc5ccccc5s4)CC3)nc3c2[S@](=O)CC3)CCC1 ; NCC1(Nc2nc(N3CC=C(c4nc5ccccc5s4)CC3)nc3c2[S@](=O)CC3)CCC1]

Step 1: Crude intermediate 56C hydrochloride (800 mg), intermediate 68J (755 mg, 2.64 mmol), and N,N-diisopropylethylamine (1.02 g, 7.92 mmol) were suspended in 1,4-dioxane (5 mL) and reacted at 80° C. for 16 h under nitrogen protection. After the reaction was complete, the system was cooled to room temperature, and the reaction liquid was dropwise added into water (10 mL) and filtered. The filter cake was slurried with ethyl acetate (5 mL), stirred at room temperature for 0.5 h, and filtered, and the solid was dried to obtain compound 78A (680 mg, 55%).

LC-MS (ESI): m/z=466.1 [M+H]⁺

Step 2: Compound 78A (370 mg, 0.79 mmol) was dissolved in methanol (2 mL), ammonium acetate (61 mg, 1.58 mmol) was added and stirred at 60° C. for 1 h, and sodium cyanoborohydride (76 mg, 1.19 mmol) was added and further reacted at 60° C. for 1 h. After the reaction was complete, the reaction liquid was concentrated, and the crude product was purified by HPLC to obtain the target compound 78 (66 mg, 1.998 min, 15%). Analysis method: instrument: Shimadzu LCMS-2020; column: Phenomenex C18; mobile phase: A was 0.1% TFA in H₂O; B was acetonitrile; gradient: Phase B from 5% to 95% in 4 min; flow rate: 1.5 mL/min; column temperature: 45° C.; detection wavelength: 220 & 254 nm;

separation method: instrument: SHIMADZU LC-20AP; preparative column: Phenomenex C18; mobile phase: A was 0.1% TFA in H2O; B was acetonitrile; gradient: Phase B from 15 to 45 in 17 min; flow rate: 25 mL/min; column temperature: room temperature; detection wavelength: 254 nm;

¹H NMR (400 MHz, DMSO-d6) δ 8.07 (d, 1H), 7.96 (d, 1H), 7.68 (s, 3H), 7.58-7.36 (m, 3H), 6.91-6.88 (m, 1H), 4.47 (s, 2H), 4.02-4.00 (m, 1H), 3.55-3.38 (m, 3H), 3.31-3.22 (m, 1H), 3.07-2.87 (m, 2H), 2.77 (s, 2H), 2.41-2.28 (m, 4H), 1.98-1.79 (m, 2H).

¹⁹F NMR (376 MHz, DMSO-d6) δ −71.96 (s).

LC-MS (ESI): m/z=467.0 [M+H]⁺

### Example 79

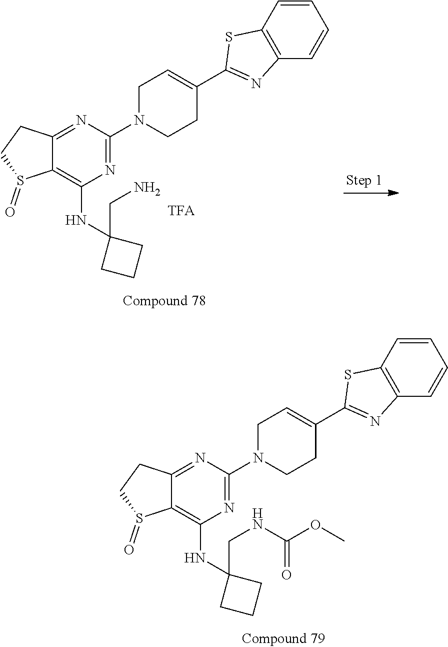 [SMILES: NCC1(Nc2nc(N3CC=C(c4nc5ccccc5s4)CC3)nc3c2[S@](=O)CC3)CCC1 ; COC(=O)NCC1(Nc2nc(N3CC=C(c4nc5ccccc5s4)CC3)nc3c2[S@](=O)CC3)CCC1]

Step 1: Compound 78 (33 mg, 0.058 mmol) and N,N-diisopropylethylamine (9 mg, 0.087 mol) were suspended in dichloromethane (2 mL), methyl chloroformate (6 mg, 0.064 mmol) was added in an ice bath, and the mixture was heated to room temperature and reacted for 1 h. After the reaction was complete as monitored by TLC, the reaction liquid was directly concentrated, and the crude product was purified by HPLC to obtain compound 79 (25 mg, 2.434 min, 82%).

Analysis method: instrument: Shimadzu LCMS-2020; column: Phenomenex C18; mobile phase: A was 0.1% NH₄CO₃ in H₂O; B was acetonitrile; gradient: Phase B from 5% to 95% in 4 min; flow rate: 1.5 mL/min; column temperature: 45° C.; detection wavelength: 220 & 254 nm; separation method: instrument: SHIMADZU LC-20AP, preparative column: Phenomenex C18; mobile phase: A was 0.1% NH₄CO₃ in H₂O; B was acetonitrile; gradient: Phase B from 26 to 56 in 17 min; flow rate: 25 mL/min; column temperature: 45° C.; detection wavelength: 254 nm;

¹H NMR (400 MHz, DMSO-d6) δ 8.07 (d, 1H), 7.96 (d, 1H), 7.56-7.36 (m, 3H), 7.19-7.15 (m, 1H), 6.90 (s, 1H), 4.49 (s, 2H), 4.03 (t, 2H), 3.59-3.38 (m, 5H), 3.31-3.17 (m, 2H), 3.01-2.82 (m, 2H), 2.77 (s, 2H), 2.43-2.16 (m, 4H), 1.91-1.69 (m, 2H).

LC-MS (ESI): m/z=525.1 [M+H]⁺

### Example 80

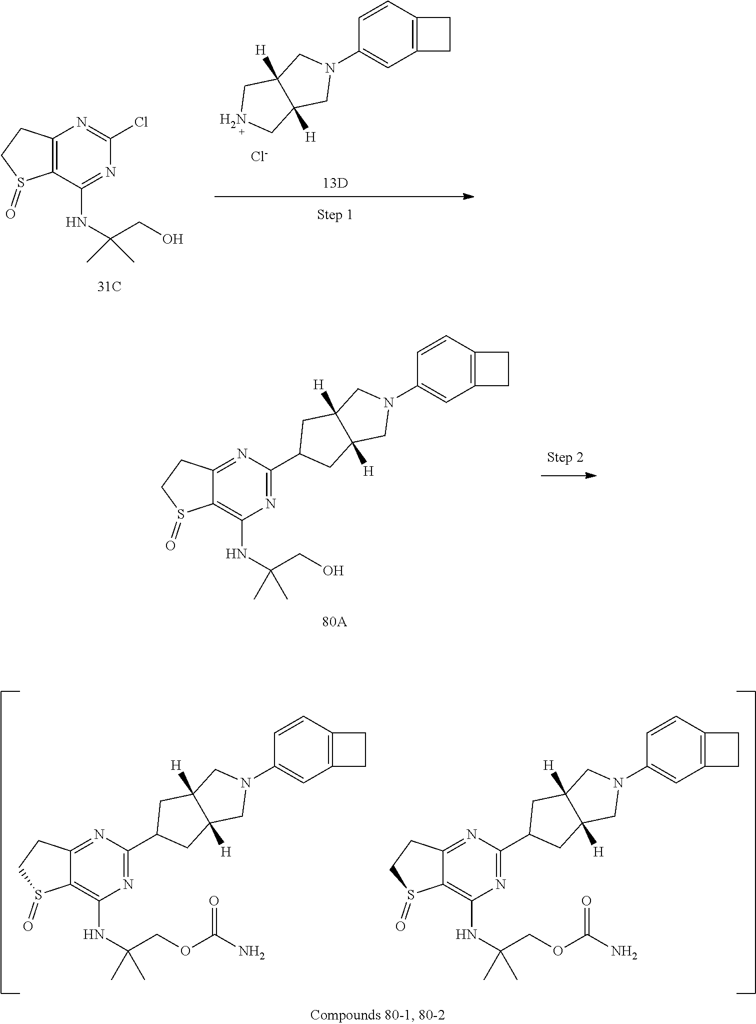

Using compounds 13D and 31C as raw materials, compounds 80-1 (5.6 mg, 2%, 2.257 min) and 80-2 (32.2 mg, 12%, 2.700 min) were obtained according to the operation of Example 49.

Chiral separation method: instrument: Waters 150 Prep-SFC E, chiral column: Chiralcel OJ column mobile phase: A for CO₂; B for 0.1% NH₃·H₂O in MEOH and CAN, gradient: B 40%, flow rate: 120 mL/min, column pressure: 100 bar, column temperature: 25° C., detection wavelength: 220 nm, cycle time: 2.3 min.

### (Compound 80-1)

¹H NMR (400 MHz, DMSO-d6) δ 6.87-6.83 (m, 1H), 6.42-6.32 (m, 2H), 6.10 (s, 1H), 5.15 (s, 1H), 3.82 (s, 2H), 3.55-3.38 (m, 6H), 3.38-3.25 (m, 3H), 3.20-2.96 (m, 8H), 2.98-2.77 (m, 2H), 1.40 (s, 6H).

LC-MS (ESI): m/z=497.1 [M+H]⁺

### (Compound 80-2)

¹H NMR (400 MHz, DMSO-d6) δ 6.87-6.83 (m, 1H), 6.42-6.32 (m, 2H), 6.10 (s, 1H), 5.15 (s, 1H), 3.82 (s, 2H), 3.55-3.38 (m, 6H), 3.38-3.25 (m, 3H), 3.20-2.96 (m, 8H), 2.98-2.77 (m, 2H), 1.40 (s, 6H).

LC-MS (ESI): m/z=497.1 [M+H]⁺

### Example 81

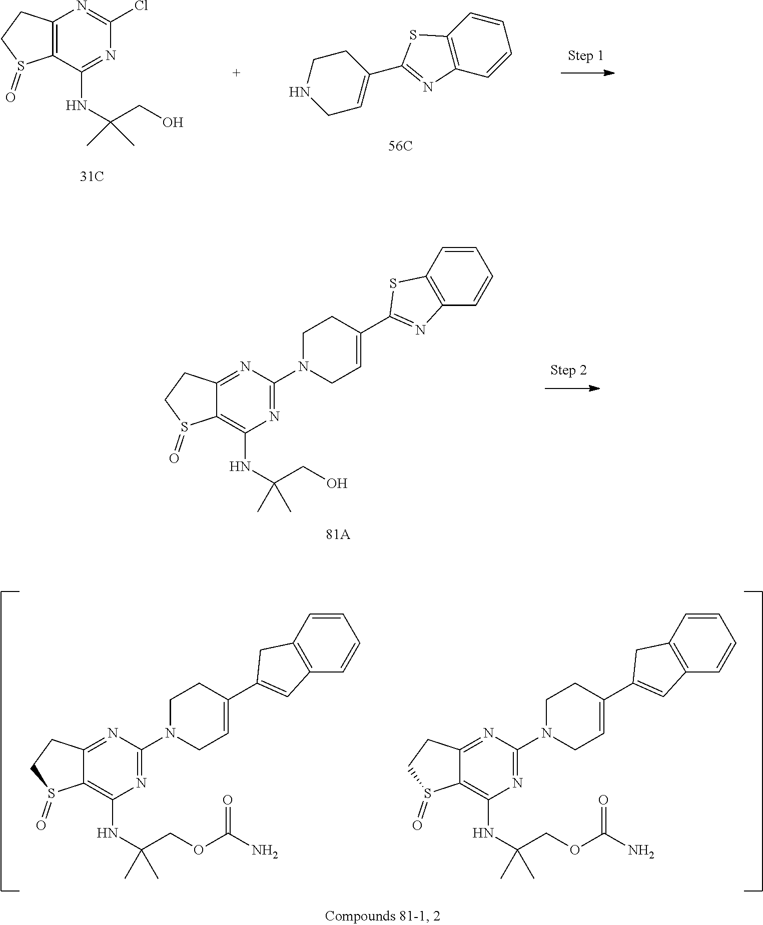

Using compounds 56C and 31C as raw materials, compounds 81-1 (3.6 mg, 2%) and 81-2 (21.2 mg, 10%) were obtained according to the operation of Example 49.

### Chiral Separation Method:

instrument: Waters 150 Prep-SFC F, chiral column: Chiralcel Cellulose-2 Column;
mobile phase: A for CO₂; B for 0.1% NH₃·H₂O in MeOH and CAN, gradient: B 60%,
flow rate: 100 mL/min, column pressure: 100 bar, column temperature: 25° C., detection wavelength: 220 nm, cycle time: 5.0 min.

### Compound 81-1:

retention time: 0.856 min, ¹H NMR (400 MHz, DMSO-d6) δ 8.07 (d, 1H), 7.97 (d, 1H), 7.54-7.37 (m, 2H), 6.91 (s, 1H), 6.67 (s, 1H), 6.56 (s, 2H), 4.51 (s, 2H), 4.31 (s, 2H), 4.07-4.03 (m, 2H), 3.43-3.28 (m, 2H), 3.04-2.84 (m, 2H), 2.79 (s, 2H), 1.47 (d, 6H).

LC-MS (ESI): m/z=499.1 [M+H]⁺

### Compound 81-2:

retention time: 1.058 min. ¹H NMR (400 MHz, DMSO-d6) δ 8.07 (d, 1H), 7.97 (d, 1H), 7.54-7.37 (m, 2H), 6.91 (s, 1H), 6.67 (s, 1H), 6.56 (s, 2H), 4.51 (s, 2H), 4.31 (s, 2H), 4.07-4.03 (m, 2H), 3.43-3.28 (m, 2H), 3.04-2.84 (m, 2H), 2.79 (s, 2H), 1.47 (d, 6H).

LC-MS (ESI): m/z=499.1 [M+H]⁺

### Example 82

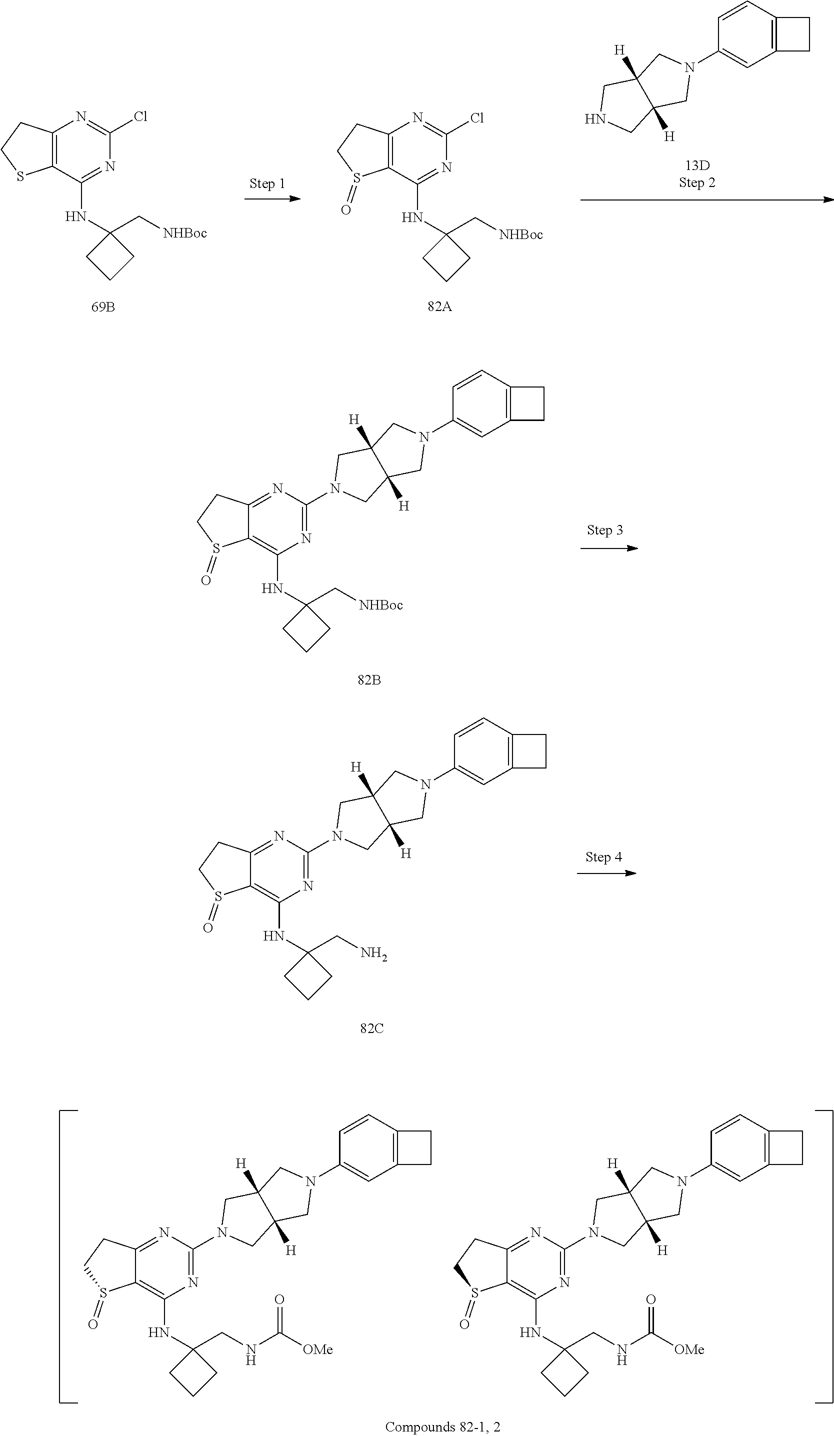

Step 1: Compound 69B (766.9 mg, 2.07 mmol), 1,1′-bi-2-naphthol (58.7 mg, 0.021 mmol), titanium tetraisopropoxide (29.4 mg, 0.011 mmol), and water (32.0 mg, 1.78 mmol) were dissolved in dichloromethane (20 mL). After nitrogen displacement three times, the system was stirred at room temperature for one hour, and tert-butyl hydroperoxide (293 mg, 2.28 mmol, 70% in water) was then added and stirred overnight. After quenching with a saturated sodium thiosulfate aqueous solution, the aqueous phase was extracted with dichloromethane, dried, concentrated, and subjected to column chromatography (dichloromethane/methanol (v/v)=91/9) to obtain compound 82A (480 mg, 60%).

Using compounds 82A and 13D as raw materials, compounds 82-1 (23.1 mg, 8%, with absolute configuration unknown) and 82-2 (20.2 mg, 7%, with absolute configuration unknown) were obtained according to the operation of Example 69.

Chiral separation method: instrument: Instrument: Waters 150 Prep-SFC F, chiral column: Chiralcel Whelk-Column mobile phase: A for CO₂; B for 0.1% NH₃·H₂O in MEOH and CAN, gradient: B 45%; flow rate: 100 mL/min, column pressure: 100 bar, column temperature: 25° C., detection wavelength: 220 nm, cycle time: 8.0 min.

### Compound 82-1:

retention time: 1.173 min, ¹H NMR (400 MHz, DMSO-d6) δ 7.36 (s, 1H), 7.12 (s, 1H), 6.84 (m, 1H), 6.38-6.33 (m, 2H), 3.79 (s, 2H), 3.58-3.32 (m, 10H), 3.19-2.97 (m, 8H), 2.97-2.80 (m, 2H), 2.44-2.22 (m, 3H), 2.17 (s, 2H), 1.85-1.65 (m, 2H).

LC-MS (ESI): m/z=523.2 [M+H]⁺

### Compound 82-2:

retention time: 1.360 min. ¹H NMR (400 MHz, DMSO-d6) δ 7.36 (s, 1H), 7.12 (s, 1H), 6.84 (m, 1H), 6.38-6.33 (m, 2H), 3.79 (s, 2H), 3.58-3.32 (m, 10H), 3.19-2.97 (m, 8H), 2.97-2.80 (m, 2H), 2.44-2.22 (m, 3H), 2.17 (s, 2H), 1.85-1.65 (m, 2H).

LC-MS (ESI): m/z=523.2 [M+H]⁺

### Example 83

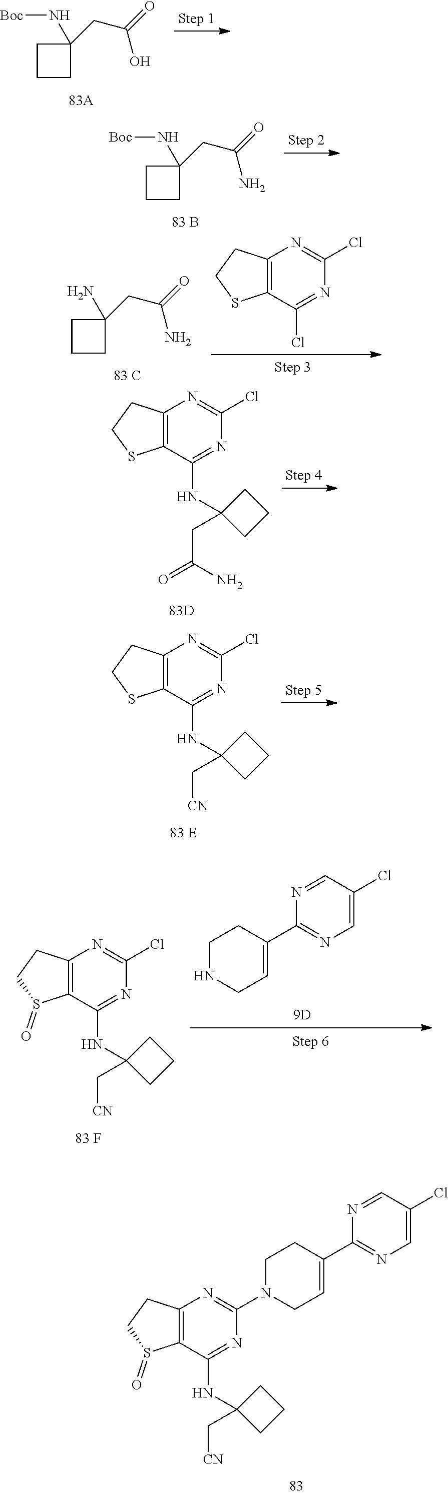

Step 1: Compound 83A (5.0 g, 21.81 mmol) was dissolved in acetonitrile (50 mL), pyridine (5.18 g, 65.42 mmol) and di-tert-butyl dicarbonate (7.14 g, 32.71 mmol) were then added and stirred at room temperature for 4 hours, and aqueous ammonia (10 mL) was dropwise added. After the addition was complete, the system was reacted at room temperature for 4 hours. After the reaction was complete, the system was concentrated, a solid was filtered off, and after washing with acetonitrile, compound 83B (4.5 g, 90%) was obtained.

LC-MS (ESI): m/z=173.1 [M+H-56]⁺

Step 2: Compound 83B (4.5 g, 19.71 mmol) was dissolved in dichloromethane (40 mL), and trifluoroacetic acid (10 mL) was added and stirred at room temperature for 1 hour. The reaction liquid was concentrated to obtain crude compound 83C trifluoroacetate, which was directly used in the next reaction without purification.

LC-MS (ESI): m/z=129.1 [M+H]⁺.

Step 3: The crude compound 83C trifluoroacetate in the previous step was dissolved in N,N-dimethylformamide (50 mL), N,N-diisopropylethylamine (6.12 g, 29.57 mmol) and 2,4-dichloro-6,7-dihydrothieno[3,2-d]pyrimidine (12.74 g, 6.87 mmol) were added, and the mixture was heated to 80° C. and reacted for 16 h. The reaction liquid was cooled to room temperature, diluted with ethyl acetate, and washed with water, and the organic phase was dried, filtered, concentrated, and subjected to column chromatography (dichloromethane/methanol (v/v)=91/9) to obtain compound 83D (720 mg, 12%).

LC-MS (ESI): m/z=299.2 [M+H]⁺

Step 4: Compound 83D in the previous step was dissolved in dichloromethane (30 mL), triethylamine (490 mg, 4.82 mmol) was added in an ice bath and stirred for five minutes, and trifluoroacetic anhydride (760 mg, 3.62 mmol) was then dropwise added. After the addition was complete, the mixture was stirred in the ice bath for 2 hours. The reaction liquid was quenched by adding water and extracted with dichloromethane, and the organic phase was dried, filtered, concentrated, and subjected to column chromatography (dichloromethane/methanol (v/v)=91/9) to obtain compound 83E (440 mg, 65%).

LC-MS (ESI): m/z=281.2 [M+H]⁺

Step 5: Compound 83E (500 mg, 1.78 mmol), (S)-(−)-1,1′-bi-2-naphthol (51.0 mg, 0.18 mmol), titanium tetraisopropoxide (25.0 mg, 0.088 mmol), and water (32.0 mg, 1.78 mmol) were dissolved in dichloromethane (20 mL). After nitrogen displacement three times, the system was stirred at room temperature for one hour, and tert-butyl hydroperoxide (256 mg, 1.99 mmol, 70% in water) was then added and stirred overnight. After quenching with a saturated sodium thiosulfate aqueous solution, the aqueous phase was extracted with dichloromethane, dried, concentrated, and subjected to column chromatography (dichloromethane/methanol (v/v)=91/9) to obtain compound 83F (520 mg, 98%).

LC-MS (ESI): m/z=297.1 [M+H]⁺

Step 6: Compound 83F (520 mg, 1.75 mmol) was dissolved in 1,4-dioxane (20 mL), and compound 9D (410 mg, 2.10 mmol) and N,N-diisopropylethylamine (680 mg, 5.27 mmol) were then added and stirred at 90° C. overnight under nitrogen protection. After the reaction was complete, the reaction liquid was concentrated and subjected to reverse phase preparation (acetonitrile:water=1/99-99/1) to obtain compound 83 (470 mg, 59%).

¹H NMR (400 MHz, DMSO-d₆) δ 8.89 (s, 2H), 8.17 (s, 1H), 7.39-7.21 (m, 1H), 4.45 (s, 2H), 3.97 (s, 2H), 3.53-3.41 (m, 1H), 3.37 (d, 2H), 3.27-3.16 (m, 1H), 3.04-2.85 (m, 2H), 2.66 (s, 2H), 2.48-2.32 (m, 2H), 2.30-2.20 (m, 2H), 2.02-1.75 (m, 2H).

LC-MS (ESI): m/z=456.5 [M+H]⁺

### Example 84

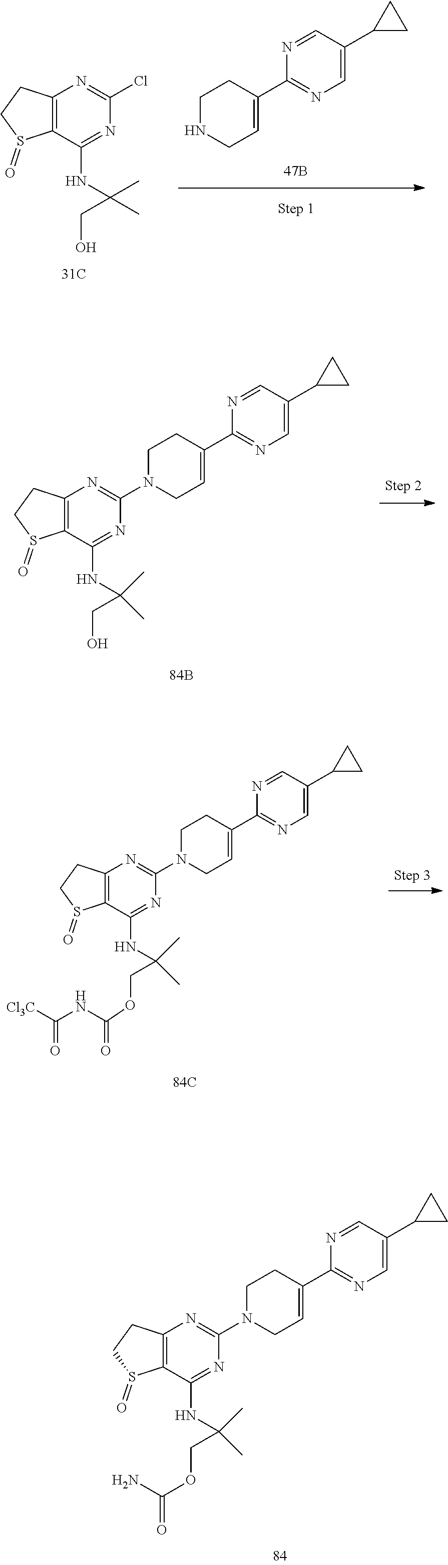

Step 1: Compound 31C (1.0 g, 3.63 mmol) was dissolved in 1,4-dioxane (30 mL), and compound 47B (880 mg, 4.36 mmol) and N,N-diisopropylethylamine (1.40 g, 10.86 mmol) were added and stirred at 90° C. overnight under nitrogen protection. After the reaction was complete, the reaction liquid was concentrated and subjected to silica gel column chromatography (dichloromethane/methanol (v/v)=95/5) to obtain compound 84B (1.6 g, 94%).

LC-MS (ESI): m/z=441.6 [M+H]⁺

Step 2: Compound 84B (1.6 g, 3.63 mmol) was dissolved in dichloromethane (5 mL), and trichloroacetyl isocyanate (820 mg, 4.36 mmol) was added in an ice bath and stirred in the ice bath for one hour. The reaction liquid was concentrated to obtain compound 84C (2.28 g, 100%).

Step 3: Compound 84C (2.28 g, 3.63 mmol) was dissolved in methanol (20 mL), and potassium carbonate (1.51 g, 10.89 mmol) and water (20 mL) were added in an ice bath and stirred at room temperature 2.5 hours. The reaction liquid was diluted by adding water and extracted with dichloromethane, and the organic phases were combined, dried, filtered, concentrated, and subjected to SFC chiral resolution to obtain compound 84 (1.3 g, 74%).

Preparation method: instrument: Waters 150 Prep-SFC E, column: Chiralcel OX column (250 mm×30 mm, 10 μm), mobile phase: (phase A: CO₂, phase B: EtOH (0.1% NH₃·H₂O)); gradient: 50% mobile phase B, isocratic elution; flow rate: 100 mL/min, back pressure: 100 bar, column temperature: 25° C., wavelength: 220 nm, cycle time: 7.0 min, sample preparation: sample concentration 10 mg/ml, acetonitrile solution injection: 8 ml each time. Retention time: 1.499 minutes. After separation, the fraction was dried in a water bath at 35° C. by a rotary evaporator to obtain a product. The solvent was dried by a lyophilizer at −80° C. to obtain the final product 84.

¹H NMR (400 MHz, DMSO-d₆) δ 8.54 (s, 2H), 7.19 (d, 1H), 6.70-6.40 (m, 3H), 4.46 (s, 2H), 4.30 (s, 2H), 4.00 (t, 2H), 3.48-3.38 (m, 1H), 3.28-3.23 (m, 1H), 3.01-2.92 (m, 1H), 2.92-2.82 (m, 1H), 2.67 (s, 2H), 1.98-1.88 (m, 1H), 1.46 (d, 6H), 1.10-0.96 (m, 2H), 0.91-0.77 (m, 2H).

LC-MS (ESI): m/z=484.2 [M+H]⁺

### Example 85

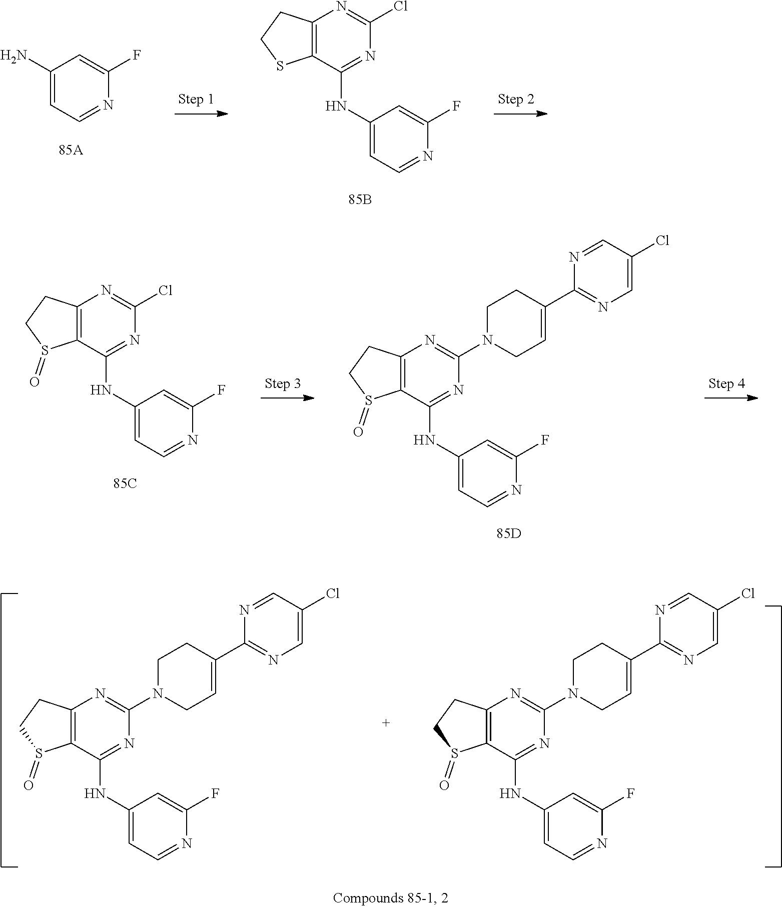

Using compounds 85A and 24A as raw materials, compound 85-1 (34.4 mg) and compound 85-2 (16.8 mg) were obtained according to the operation of Example 60.

Analysis method: instrument: SHIMADZU LC-20AD, column: Chiralpak IG-3 50×4.6 mm I.D., 3 μm; mobile phase: A: heptane (0.05%, N,N-diethylaniline), B: ethanol (0.05% N,N-diethylaniline); gradient: 20% B in A; flow rate: 1 mL/min, column temperature: 35° C., wavelength: 254 nm.

Preparation method: instrument: SHIMADZU LC-20AP, column: DAICEL CHIRALPAK IG (250 mm×30 mm, 10 μm), mobile phase: A n-hexane, B ethanol (0.1% NH₃·H₂O); gradient: 20-60% B gradient elution, flow rate: 110 mL/min, column temperature: 25° C., wavelength: 254 nm, cycle time: 16 min, sample preparation: sample concentration 1.5 mg/ml, ethanol solution injection: 2 ml each time. After separation, the fraction was dried by a rotary evaporator at a bath temperature of 40° C. to obtain compound 85-1 and compound 85-2.

### Compound 85-1:

¹H NMR (400 MHz, DMSO-d6) δ 8.91 (s, 2H), 8.14-8.12 (m, 1H), 7.80-7.75 (m, 1H), 7.63 (s, 1H), 7.33-7.32 (m, 1H), 4.66 (br s, 1H), 4.57-4.50 (m, 2H), 4.09-4.03 (m, 2H), 3.62-3.38 (m, 1H), 3.36-3.34 (m, 1H), 3.18-3.12 (m, 1H), 3.06-3.01 (m, 1H), 2.76-2.71 (m, 2H).

LCMS m/z=458.1 [M+H]⁺

### Compound 85-2:

¹H NMR (400 MHz, DMSO-d6) δ 8.91 (s, 2H), 8.14-8.12 (m, 1H), 7.78 (s, 1H), 7.63 (s, 1H), 7.33-7.32 (m, 1H), 4.66 (br s, 1H), 4.54 (s, 2H), 4.06 (br s, 2H), 3.62-3.38 (m, 1H), 3.36-3.34 (m, 1H), 3.18-3.12 (m, 1H), 3.06-3.01 (m, 1H), 2.74-2.73 (m, 2H).

LCMS m/z=458.1 [M+H]⁺

### Example 88

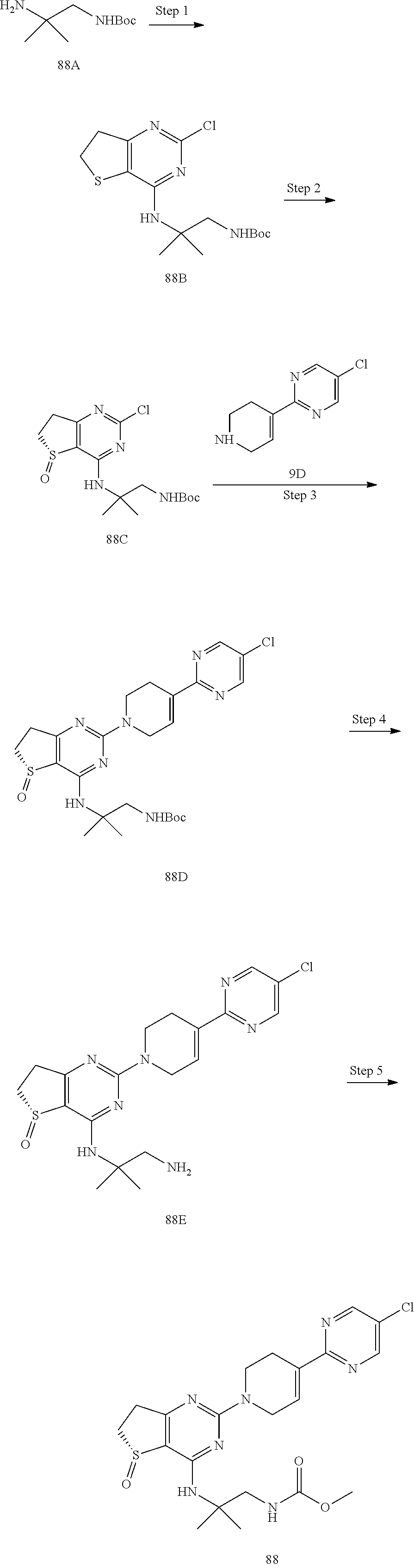

Step 1: Compound 88A (10.0 g, 53.1 mmol) was dissolved in acetonitrile (100 mL), the compound 2,4-dichloro-6,7-dihydrothieno[3,2-D]pyrimidine (11.0 g, 53.1 mmol) and triethylamine (26.6 g, 265.0 mmol) were added, heated to 75° C., and reacted for 12 h, and the system was then cooled to room temperature, concentrated, and subjected to column chromatography (dichloromethane/methanol (v/v)=92.5/7.5) to obtain compound 88B (3.5 g, 18%).

LC-MS (ESI): m/z=359.1 [M+H]⁺

Step 2: Compound 88B (3.5 g, 9.77 mmol), (S)-(−)-1,1′-bi-2-naphthol (0.28 g, 0.98 mmol), titanium tetraisopropoxide (0.13 g, 0.48 mmol), and water (0.17 g, 9.75 mmol) were dissolved in dichloromethane (25 mL). After nitrogen displacement three times, the system was stirred at room temperature for one hour, and tert-butyl hydroperoxide (0.96 mg, 10.7 mmol) was then added and stirred for 1.5 hours. After quenching with a saturated sodium thiosulfate aqueous solution, the aqueous phase was extracted with dichloromethane, dried, concentrated, and subjected to column chromatography (dichloromethane/methanol (v/v)=91/9) to obtain compound 88C (3.1 g, 95%).

LC-MS (ESI): m/z=375.1 [M+H]⁺

Step 3: Compound 88C (2.0 g, 5.3 mmol) was dissolved in 1,4-dioxane (15 mL), compound 9D (1.34 g, 5.8 mmol) and N,N-diisopropylethylamine (2.75 g, 21.2 mmol) were added, and the mixture was heated to 90° C. and reacted for 12 h. The system was then cooled to room temperature, concentrated, and subjected to column chromatography (dichloromethane/methanol (v/v)=91/9) to obtain compound 88D (2.5 g, 88%).

LC-MS (ESI): m/z=534.1 [M+H]⁺

Step 4: Compound 88D (2.5 g, 4.7 mmol) was dissolved in dichloromethane (10 mL), and trifluoroacetic acid (5.0 mL) was then added and reacted at room temperature for 2.5 hours. The system was then concentrated and subjected to column chromatography (dichloromethane/methanol (v/v)=92.5/7.5) to obtain compound 88E (1.9 g, 91%).

LC-MS (ESI): m/z=434.1 [M+H]⁺

Step 5: Compound 88E (0.9 g, 2.0 mmol) was dissolved in dichloromethane (10 mL), and triethylamine (0.4 g, 4.0 mmol) was added, followed by methyl chloroformate (0.188 g, 2.0 mmol). The mixture was reacted at room temperature for 1.5 hours, concentrated, and subjected to column chromatography (dichloromethane/methanol (v/v)=91/9), followed by chiral resolution to obtain compound 88 (0.3 g, 30%).

Preparation method: instrument: Waters 150 Prep-SFC, column: Chiralcel OX Colum, mobile phase: (phase A: CO₂, phase B: 0.1% NH₃·H₂O in MEOH and ACN); gradient: 40% mobile phase B, isocratic elution; flow rate: 120 mL/min, back pressure: 100 bar, column temperature: 25° C., wavelength: 220 nm, cycle time: 4.9 min, sample preparation: sample concentration 50 mg/ml, acetonitrile solution injection: 2 ml each time. After separation, the fraction was dried on a rotary evaporator at a bath temperature of 35° C. to obtain 88 (retention time: 1.062 minutes).

¹H NMR (400 MHz, DMSO-d6) δ 8.89 (s, 2H), 7.45 (d, 1H), 7.30 (s, 1H), 6.70 (s, 1H), 4.50 (d, 2H), 4.01 (t, 2H), 3.57 (s, 3H), 3.46-3.38 (m, 2H), 3.34 (s, 1H), 3.25-3.20 (m, 1H), 2.99-2.93 (m, 1H), 2.89-2.84 (m, 1H), 2.67 (s, 2H), 1.45 (s, 3H), 1.42 (s, 3H).

LC-MS (ESI): m/z=492.1 [M+H]⁺

### Example 89

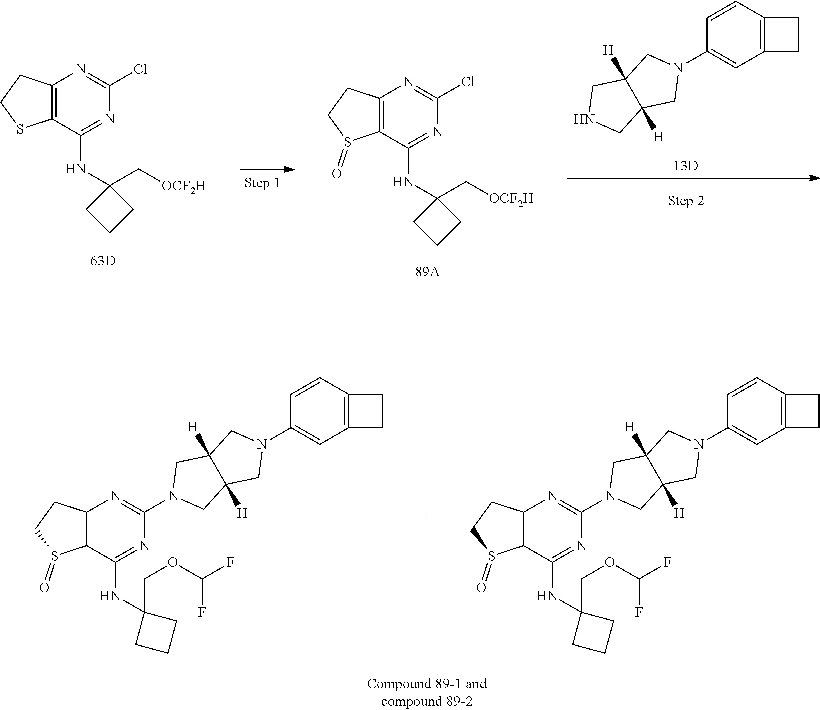

Step 1: Compound 63D (0.8 g, 2.5 mmol) was dissolved in dichloromethane (5.0 mL), and m-chloroperoxybenzoic acid (0.43 g, 2.5 mmol) was then added and reacted at room temperature for 4 h. The system was concentrated and subjected to column chromatography (dichloromethane/methanol=15/1) to obtain 89A (0.67 g, 79%).

LC-MS (ESI): m/z=338.1 [M+1]⁺

Step 2: Compound 89A (0.67 g, 2.0 mmol) was dissolved in 1,4-dioxane (15 mL), 13D (0.45 g, 2.1 mmol) and N,N-diisopropylethylamine (0.78 g, 6.0 mmol) were then added, and the mixture was heated to 90° C. and reacted for 12 h. The system was then cooled to room temperature, concentrated, and subjected to column chromatography (dichloromethane/methanol (v/v)=91/9) to obtain the target compound, which was further subjected to chiral resolution to obtain compounds 89-1 (20 mg, 2%) and 89-2 (15 mg, 1%).

Preparation method: instrument: Waters 150 Prep-SFC, column: Chiralcel OX Colum, mobile phase: (phase A: CO₂, phase B: 0.1% NH₃·H₂O in MEOH and ACN); gradient: 40% mobile phase B, isocratic elution; flow rate: 120 mL/min, back pressure: 100 bar, column temperature: 25° C., wavelength: 220 nm, cycle time: 4.9 min, sample preparation: sample concentration 50 mg/ml, acetonitrile solution injection: 2 ml each time. After separation, the fraction was dried by a rotary evaporator at a bath temperature of 35° C. to obtain compounds 89-1 (retention time: 0.952 minutes) and 89-2 (retention time: 1.062 minutes).

### Compound 89-1

¹H NMR (400 MHz, DMSO-d6) δ 7.51 (s, 1H), 6.85-6.32 (m, 4H), 4.20 (s, 2H), 3.99 (s, 2H), 3.61 (s, 2H), 3.52-3.33 (m, 3H), 3.16 (s, 5H), 3.06-2.88 (m, 6H), 2.18 (d, 4H), 1.82 (d, 2H).

¹⁹F NMR (377 MHz, DMSO-d6) δ −79.70 (s).

LC-MS (ESI): m/z=516.1 [M+H]⁺

### Compound 89-2:

¹H NMR (400 MHz, DMSO-d6) δ 7.70 (s, 1H), 6.89-6.30 (m, 4H), 4.25 (s, 2H), 3.77 (s, 2H), 3.53-3.33 (m, 5H), 3.23-2.98 (m, 9H), 2.95-2.82 (m, 2H), 2.43-2.10 (m, 4H), 1.86-1.80 (m, 2H).

¹⁹F NMR (377 MHz, DMSO-d6) δ −79.93 (s).

LC-MS (ESI): m/z=516.1 [M+H]⁺

### Example 90

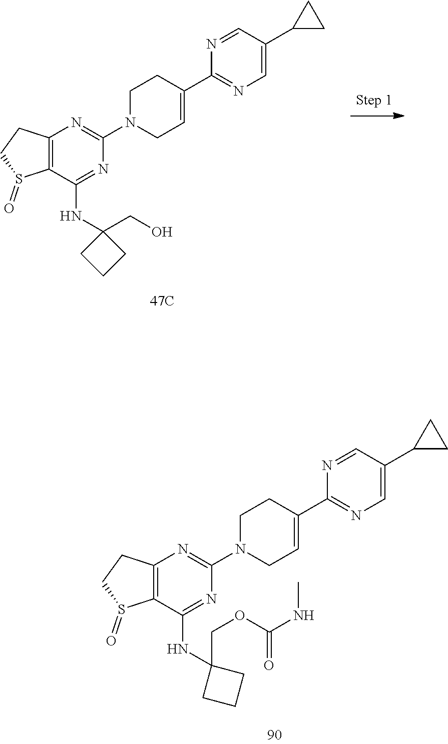 [SMILES: O=[S@@]1CCc2nc(N3CC=C(c4ncc(C5CC5)cn4)CC3)nc(NC3(CO)CCC3)c21 ; CNC(=O)OCC1(Nc2nc(N3CC=C(c4ncc(C5CC5)cn4)CC3)nc3c2[S@](=O)CC3)CCC1]

Step 1: Compound 47C (1.80 g, 4.0 mmol) was dissolved in N,N-dimethylformamide (20 mL), and triethylamine (2.4 g, 24.0 mmol) and methylaminoformyl chloride (0.5 g, 5.2 mmol) were added. After nitrogen displacement three times, the mixture was heated to 70° C. and reacted for 14 h. The system was then cooled to room temperature, concentrated, and subjected to column chromatography (dichloromethane/methanol (v/v)=95/5) to obtain compound 90 (0.3 g, 15%).

¹H NMR (400 MHz, DMSO-d6) δ 8.54 (s, 2H), 7.58 (s, 1H), 7.18 (s, 1H), 6.96-6.93 (m, 1H), 4.43 (s, 4H), 3.98-3.95 (m, 2H), 3.48-3.39 (m, 1H), 3.25-3.17 (m, 1H), 2.99-2.93 (m, 1H), 2.89-2.84 (m, 1H), 2.64 (s, 2H), 2.56 (d, 3H), 2.43-2.32 (m, 2H), 2.29-2.21 (m, 2H), 1.97-1.90 (m, 1H), 1.86-1.82 (m, 2H), 1.05-1.01 (m, 2H), 0.86-0.81 (m, 2H).

LC-MS (ESI): m/z=510.6 [M+H]⁺

### Example 91

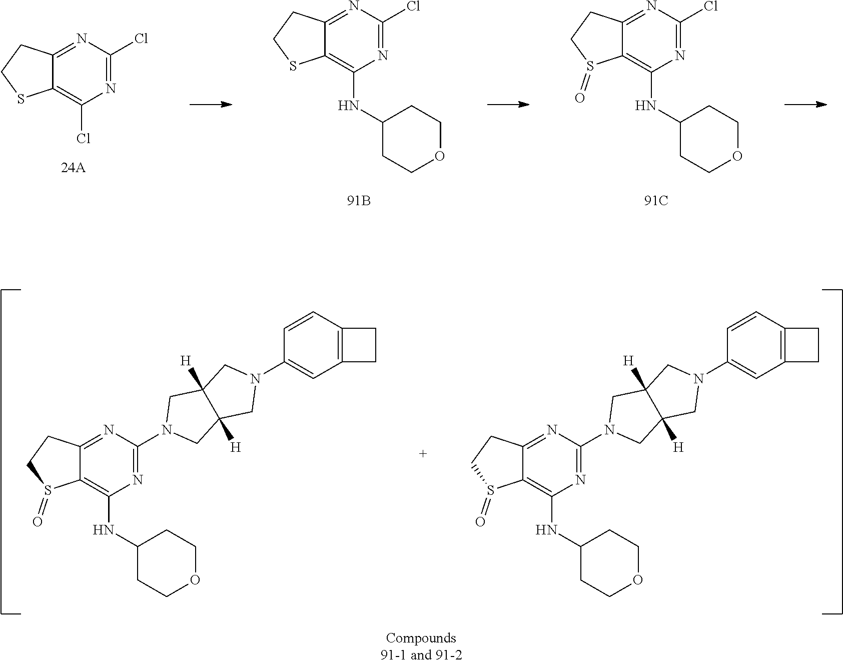

Step 1: At room temperature, compound 24A (1.0 g, 4.83 mmol), 4-aminotetrahydropyran (0.59 g, 5.80 mmol), and acetonitrile (15 mL) were added to a 50 mL round-bottom flask, followed by triethylamine (1.22 g, 12.1 mmol), and the mixture was heated to 70° C. and reacted for 14 h. After the reaction was complete, the system reaction liquid was cooled to room temperature, concentrated under reduced pressure, and then separated and purified by silica gel column chromatography (petroleum ether/ethyl acetate (v/v)=100/0-65/35) to obtain the target compound 91B (1.05 g, 80%).

LC-MS (ESI): m/z=272.1 [M+H]⁺

Step 2: At room temperature, 91B (1.05 g, 3.86 mmol) and dichloromethane (10 mL) were added to a 50 mL round-bottom flask, followed by m-chloroperoxybenzoic acid (1.0 g, 5.81 mmol) slowly, and the mixture was continuously stirred at this temperature for 16 h. After the reaction was complete, the reaction was quenched with a saturated sodium thiosulfate aqueous solution. The aqueous phase was extracted twice with dichloromethane (20 mL), and the organic phases were combined, washed with a saturated sodium chloride aqueous solution, and dried over anhydrous sodium sulfate. After filtration, the reaction liquid was concentrated under reduced pressure and then separated and purified by silica gel column chromatography (petroleum ether/ethyl acetate (v/v)=100/0-50/50) to obtain the target compound 91C (0.95 g, 85%).

LC-MS (ESI): m/z=288.3 [M+H]⁺

Step 3: At room temperature, compound 91C (260 mg, 0.90 mmol), compound 13D (213 mg, 0.99 mmol), and 1,4-dioxane (5 mL) were added to a 25 mL round-bottom flask, and N,N-diisopropylethylamine (347 mg, 2.71 mmol) were added at room temperature, heated to 90° C. and reacted for 12 h. After the reaction was complete, the reaction liquid was concentrated under reduced pressure and then separated and purified by silica gel column chromatography (dichloromethane/methanol (v/v)=91/9) to obtain 230 mg of a racemate, which was separated by SFC to obtain two isomers, i.e., compound 91-1 (100 mg, retention time 1.15 min, 24%) and compound 91-2 (100 mg, retention time 1.91 min, 24%).

Separation conditions of preparative chromatography: 1. instrument: Waters 150 Prep-SFC F; 2. chromatographic column: Chiralcel OX Column; 3. mobile phase system: A for CO₂; B for 0.1% NH₃·H₂O in MEOH and ACN; 4. gradient: B 70%; 5. flow rate: 100 mL/min.

### Compound 91-1:

¹H NMR (400 MHz, Chloroform-d) δ 6.90 (d, 1H), 6.42-6.38 (m, 1H), 6.37-6.33 (m, 1H), 5.64-5.45 (m, 1H), 4.25-4.14 (m, 1H), 4.02-3.94 (m, 2H), 3.92-3.84 (m, 2H), 3.65-3.44 (m, 7H), 3.41-3.32 (m, 1H), 3.27-3.16 (m, 2H), 3.12-3.06 (m, 6H), 3.05-2.95 (m, 2H), 2.03-1.93 (m, 2H), 1.65-1.51 (m, 2H).

LC-MS (ESI): m/z=466.7 [M+H]⁺

### Compound 91-2:

¹H NMR (400 MHz, Chloroform-d) δ 6.90 (d, 1H), 6.43-6.38 (m, 1H), 6.37-6.33 (m, 1H), 5.56-5.31 (m, 1H), 4.26-4.15 (m, 1H), 4.03-3.94 (m, 2H), 3.93-3.83 (m, 2H), 3.67-3.44 (m, 7H), 3.42-3.32 (m, 1H), 3.28-3.17 (m, 2H), 3.13-3.06 (m, 6H), 3.05-2.95 (m, 2H), 2.04-1.94 (m, 2H), 1.65-1.52 (m, 2H).

LC-MS (ESI): m/z=466.7 [M+H]⁺

### Example 92

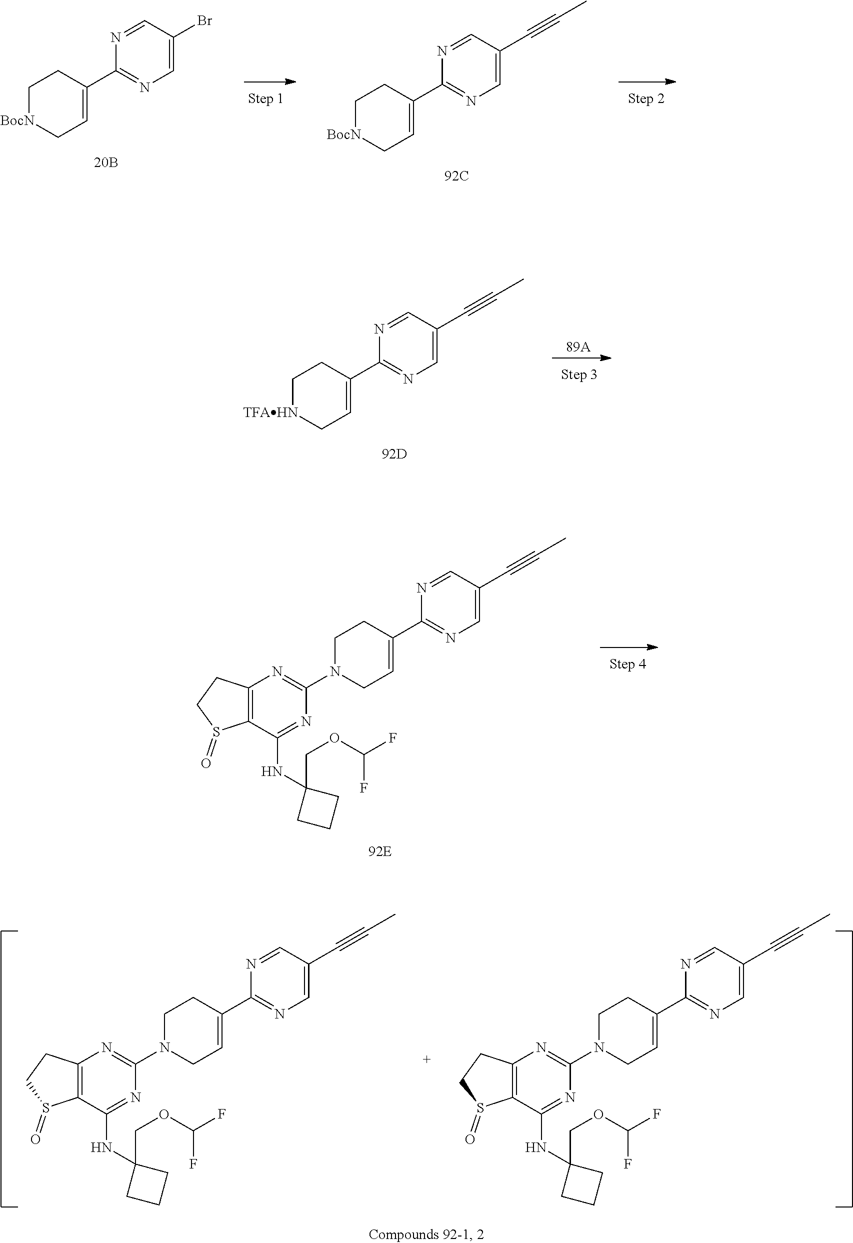

Step 1: Compound 20B (1.00 g, 2.94 mmol), 1-(trimethylsilyl)propyne (660 mg, 5.88 mmol), potassium carbonate (1.02 g, 7.35 mmol), cuprous iodide (112 mg, 0.59 mmol), and [1,1′-bis(diphenylphosphine)ferrocene]palladium dichloride (160 mg, 0.44 mmol) were added to a reaction flask, followed by 20 mL of a 1,4-dioxane solution, and after nitrogen displacement, the mixture was heated to 85° C. and reacted for 16 h. After the raw material disappeared as detected by TLC, the mixture was diluted by adding water (100 mL) and extracted three times with ethyl acetate (100 mL). The organic phases were combined, dried over anhydrous sodium sulfate, and then concentrated in vacuum to obtain a crude product, and the crude product was purified three times by column chromatography (eluents: PE:EA=100:1 to 20:1) to obtain the target compound 92C (200 mg, yield: 22.73%).

LC-MS (ESI): m/z=244.10 [M−56+H]⁺

¹H NMR (400 MHz, CDCl₃) δ 8.65 (s, 2H), 7.20-7.19 (m, 1H), 4.18-4.17 (m, 2H), 3.64-3.61 (m, 2H), 2.69-2.68 (m, 2H), 1.49 (s, 9H).

Step 2: Compound 92C (200 mg, 0.67 mmol) was dissolved in 8 mL of dichloromethane, trifluoroacetic acid (2 mL) was added, and the mixture was then stirred at 0° C. for 30 min. After the raw material disappeared as detected by TLC, the reaction liquid was concentrated at room temperature to obtain crude 92D (230 mg), which was directly used for the next reaction.

LC-MS (ESI): m/z=200.20 [M+H]⁺

Step 3: 89A (200 mg, 0.59 mmol) was dissolved in 1,4-dioxane (8 mL), and 92D (260 mg, 0.88 mmol) and N,N-diisopropylethylamine (310 mg, 2.38 mmol) were successively added. After the addition was complete, the reaction liquid was heated to 85° C. and stirred for 16 h under nitrogen protection. After the reaction liquid naturally returned to room temperature, the reaction was diluted by adding water (40 mL) and extracted three times with ethyl acetate (50 mL), and the organic phases were combined, dried over anhydrous sodium sulfate, and then concentrated in vacuum to obtain a crude product, which was purified by column chromatography (dichloromethane/methanol (v/v)=99/1-91/9) to obtain the target compound 92E (250 mg, 84.35%).

LC-MS (ESI): m/z=501.2 [M+H]⁺

Step 4: Compound 92E (250 mg, 0.50 mmol) was subjected to chiral resolution to obtain compound 92-1 (57.7 mg) and compound 92-2 (2.0 mg).

Analysis method: instrument: SHIMADZU LC-30AD, column: Chiralcel Whelk Column; mobile phase: A: CO₂, B: 0.05% DEA in IPA and ACN; gradient: 50% B in A; flow rate: 3 mL/min, column temperature: 35° C., wavelength: 220 nm.

Preparation method: instrument: Waters 150 Prep-SFC F, column: Chiralcel Whelk Column (250 mm×30 mm, 10 μm) mobile phase: A: CO₂, B: 0.1% NH₃·H₂O in IPA and ACN; gradient: 45% B gradient elution, flow rate: 100 mL/min, column temperature: 25° C., wavelength: 220 nm, cycle time: 2.7 min, sample preparation: sample concentration 5 mg/ml, ethanol solution injection: 1 ml each time. After separation, the fraction was dried by a rotary evaporator at a bath temperature of 35° C. to obtain compound 92-1 and compound 92-2.

### Compound 92-1:

¹H NMR (400 MHz, CDCl₃) δ 8.66 (s, 2H), 7.29-7.27 (m, 1H), 6.42-6.04 (m, 1H), 5.54 (s, 1H), 4.49 (s, 2H), 4.36-4.33 (m, 1H), 4.19-4.17 (m, 1H), 4.07-4.01 (m, 2H), 3.65-3.57 (m, 1H), 3.44-3.37 (m, 1H), 3.06-2.98 (m, 2H), 2.77 (s, 2H), 2.45-2.27 (m, 4H), 2.11 (s, 3H), 2.02-1.87 (m, 2H).

LC-MS (ESI): m/z=501.2 [M+H]⁺

### Compound 92-2:

¹H NMR (400 MHz, CDCl₃) δ 8.66 (s, 2H), 7.29-7.27 (m, 1H), 6.42-6.04 (m, 1H), 5.54 (s, 1H), 4.54-4.45 (m, 2H), 4.36-4.33 (m, 1H), 4.19-4.17 (m, 1H), 4.07-4.01 (m, 2H), 3.65-3.57 (m, 1H), 3.44-3.37 (m, 1H), 3.06-2.98 (m, 2H), 2.77 (s, 2H), 2.45-2.27 (m, 4H), 2.11 (s, 3H), 2.02-1.87 (m, 2H).

LC-MS (ESI): m/z=501.2 [M+H]⁺

### Example 93

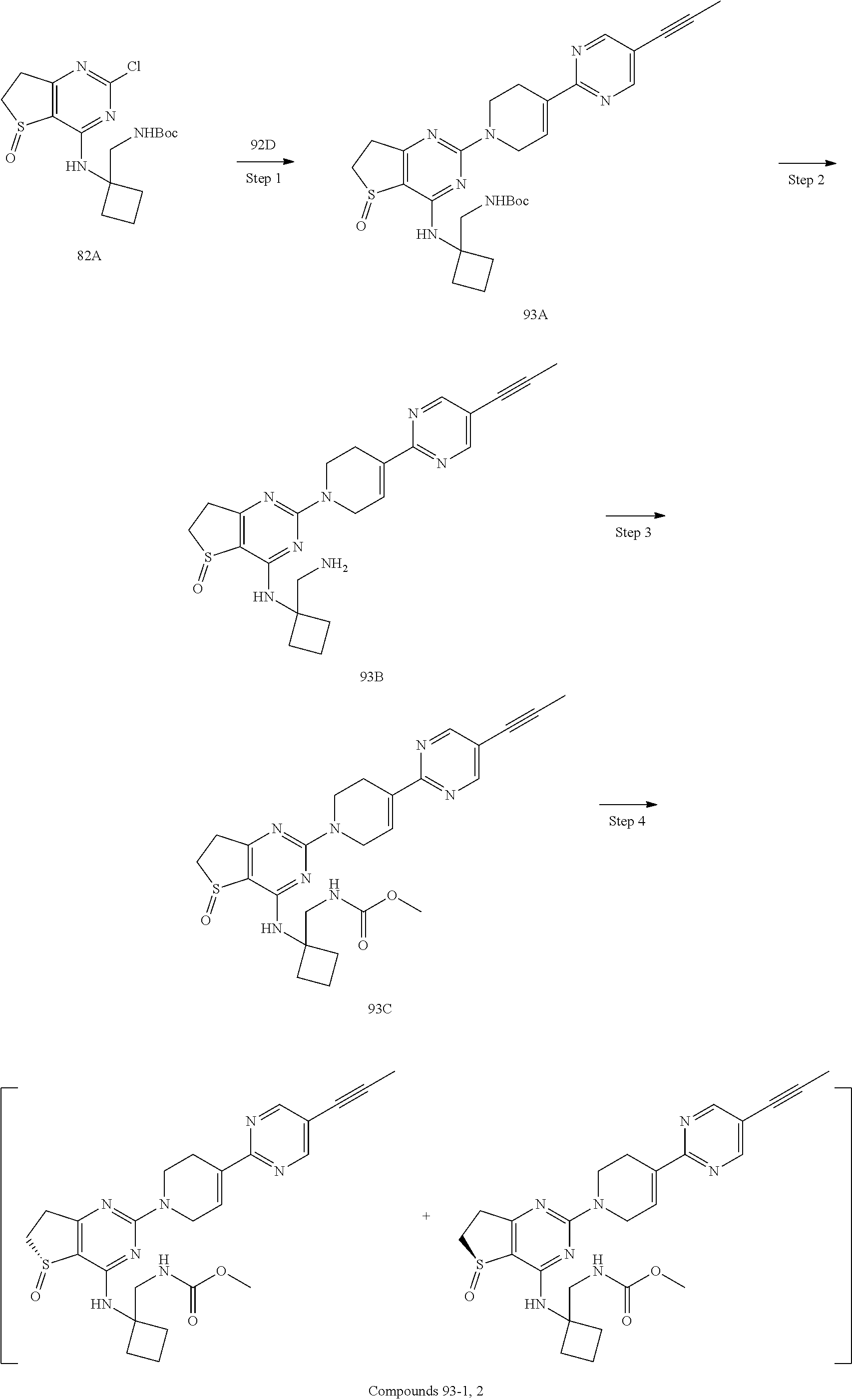

Using compounds 82A and 92D as raw materials, compound 93-1 and compound 93-2 were obtained according to the operation of Example 69.

### Compound 93-1:

¹H NMR (400 MHz, CDCl₃) δ 8.66 (s, 2H), 7.29-7.28 (m, 1H), 5.76-5.71 (m, 2H), 4.51 (s, 2H), 4.05 (s, 2H), 3.75-3.68 (m, 2H), 3.64 (s, 3H), 3.63-3.57 (m, 1H), 3.43-3.39 (m, 1H), 3.07-2.99 (m, 2H), 2.78 (s, 2H), 2.35-2.25 (m, 4H), 2.11 (s, 3H), 1.97-1.92 (m, 2H).

LC-MS (ESI): m/z=508.6 [M+H]⁺

### Compound 93-2:

¹H NMR (400 MHz, CDCl₃) δ 8.66 (s, 2H), 7.29-7.28 (m, 1H), 5.76-5.71 (m, 2H), 4.51 (s, 2H), 4.09-4.04 (m, 2H), 3.75-3.68 (m, 2H), 3.64 (s, 3H), 3.63-3.57 (m, 1H), 3.43-3.39 (m, 1H), 3.07-2.99 (m, 2H), 2.78 (s, 2H), 2.35-2.25 (m, 4H), 2.11 (s, 3H), 1.97-1.92 (m, 2H).

LC-MS (ESI): m/z=508.6 [M+H]⁺

### Example 94

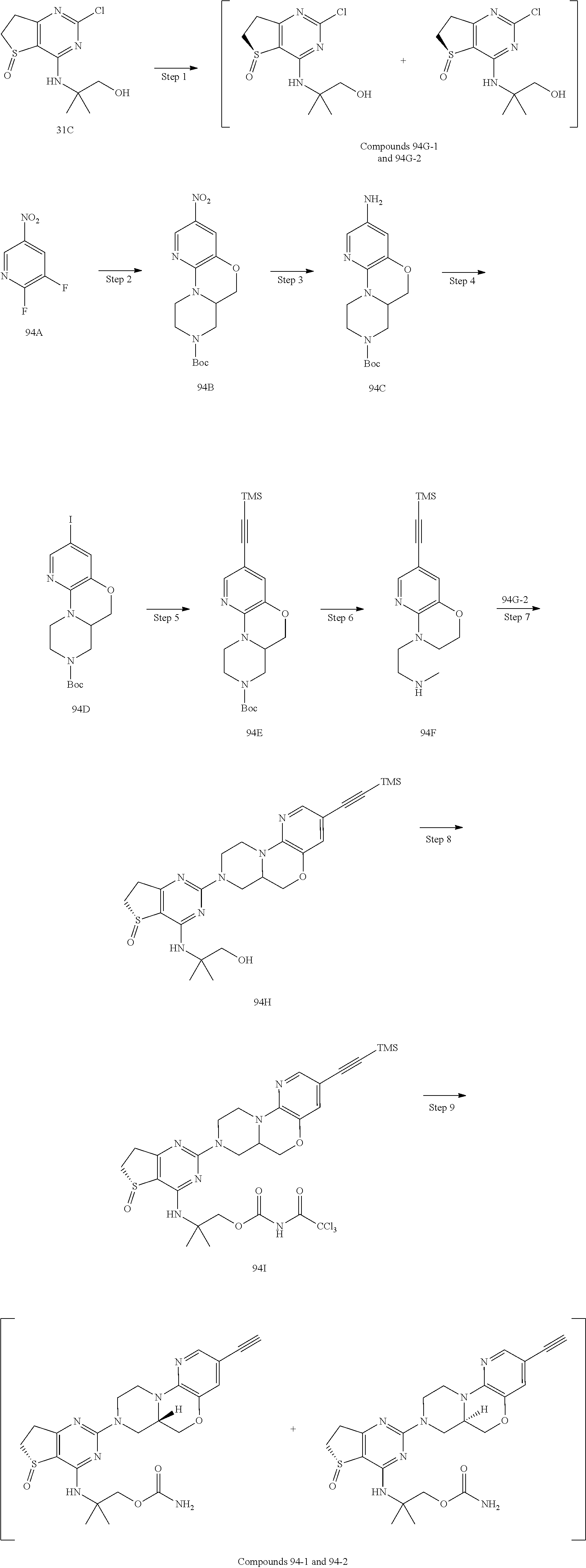

Step 1: Compound 31C (3.0 g) was separated by SFC to obtain two isomers, i.e., compound 94G-1 (1.35 g, retention time 3.50 min, 45%) and compound 94G-2 (1.45 g, retention time 4.20 min, 48%).

Separation conditions of preparative chromatography: 1. instrument: Waters 150 Prep-SFC E; 2. chromatographic column: Chiralcel IC column; 3. mobile phase system: A for CO₂; B for 0.1% NH₃·H₂O in MEOH; 4. gradient: B 40%; and 5. flow rate: 180 mL/min.

LC-MS (ESI): m/z=276.1 [M+H]⁺.

Step 2: At room temperature, compound 91A (5.0 g, 31.3 mmol), tert-butyl 3-(hydroxymethyl)piperazine-1-carboxylate (6.8 g, 31.3 mmol), potassium hydroxide (5.3 g, 93.9 mmol), and N,N-dimethylformamide (100 mL) were successively added. The mixture was heated to 100° C. and reacted for 15 h. After the reaction was complete, the reaction liquid was filtered, and the filtrate was extracted with ethyl acetate (500 mL). The organic phase was washed with water (100 mL×3) and with a saturated sodium chloride aqueous solution (100 mL), and the organic phase was dried over anhydrous sodium sulfate. After filtration, the filtrate was concentrated under reduced pressure to obtain 94B (10 g, 95%).

LC-MS (ESI): m/z=281.1 [M−^{t}Bu+H]⁺

Step 3: At room temperature in a hydrogen atmosphere, compound 94B (10 g, 29.76 mmol), palladium on carbon (2 g), and ethyl acetate (300 mL) were added to a 1 L round-bottom flask and reacted at room temperature for 12 hours. After the reaction was complete, the reaction liquid was filtered, and the filtrate was concentrated under reduced pressure to obtain 94C (8.0 g, 88%).

LC-MS (ESI): m/z=307.1 [M+H]⁺

Step 4: At room temperature, tert-butyl nitrite (3.5 g, 33.83 mmol), cuprous iodide (5.2 g, 27.06 mmol), and acetonitrile (30 mL) were successively added to a 100 mL round-bottom flask and heated to 65° C., and a solution of 94C (6.9 g, 22.55 mmol) in acetonitrile (40 mL) was dropwise added. After the dropwise addition was complete, the mixture was reacted at 65° C. for 6 h. After the reaction was complete, the reaction liquid was cooled to room temperature, concentrated under reduced pressure, and then separated and purified by silica gel column chromatography (eluents EA/PE (v/v)=0-20%) to obtain 94D (1.5 g, 16%).

LC-MS (ESI): m/z=418.4 [M+H]⁺

Step 5: At room temperature in a nitrogen atmosphere, compound 94D (600 mg, 1.44 mmol), trimethylsilylacetylene (160 mg, 1.58 mmol), bis(triphenylphosphine)palladium dichloride (98 mg, 0.14 mmol), cuprous iodide (55 mg, 0.29 mmol), and tetrahydrofuran (10 mL) were added to a 50 mL round-bottom flask, followed by triethylamine (436 mg, 4.32 mmol), and the mixture was reacted at this temperature for 12 h. After the reaction was complete, water (5 mL) was added, and the mixture was extracted twice with ethyl acetate (10 mL). The organic phases were combined, washed with a saturated sodium chloride aqueous solution, and dried over anhydrous sodium sulfate. After filtration, the reaction liquid was concentrated under reduced pressure and then separated and purified by silica gel column chromatography (eluents: EA/PE (v/v)=0-30%) to obtain the target compound 94E (450 mg, 81%).

LC-MS (ESI): m/z=388.4 [M+H]⁺

Step 6: At room temperature, 94E (450 mg, 1.16 mmol), dichloromethane (3 mL), and trifluoroacetic acid (1 mL) were added to a 25 mL round-bottom flask, and the mixture was continuously stirred at this temperature for 2 h. After the reaction was complete, the reaction liquid was concentrated under reduced pressure to obtain the target compound 94F (325 mg, 97%).

LC-MS (ESI): m/z=288.3 [M+H]⁺

Step 7: At room temperature, compound 94F (200 mg, 0.70 mmol), compound 94G-2 (211 mg, 0.76 mmol), and 1,4-dioxane (5 mL) were added to a 25 mL round-bottom flask, and N,N-diisopropylethylamine (270 mg, 2.09 mmol) were added at room temperature, heated to 100° C. and reacted for 12 h. After the reaction was complete, the reaction liquid was concentrated under reduced pressure and then separated and purified by silica gel column chromatography (eluents: MeOH/DCM (v/v)=0-10%) to obtain the target compound 94H (185 mg, 50%).

LC-MS (ESI): m/z=527.7 [M+H]⁺

Step 8: At room temperature, compound 94H (185 mg, 0.35 mmol) and dichloromethane (5 mL) were added to a 25 mL round-bottom flask, and trichloroacetyl isocyanate (79 mg, 0.42 mmol) was added in an ice bath and stirred in the ice bath for 1 h. After the reaction was complete, the reaction liquid was concentrated under reduced pressure and then separated and purified by silica gel column chromatography (eluents: MeOH/DCM (v/v)=0-10%) to obtain the target compound 941 (160 mg, 64%).

LC-MS (ESI): m/z=714.2 [M+H]⁺

Step 9: At room temperature, compound 941 (160 mg, 0.22 mmol) and methanol (2 mL) were added to a 25 mL round-bottom flask, and potassium carbonate (92 mg, 0.66 mmol) and water (2 mL) were added in an ice bath and reacted at room temperature for 2.5 h. After the reaction was complete, the reaction liquid was concentrated under reduced pressure and then separated and purified by silica gel column chromatography (eluents: MeOH/DCM (v/v)=0-10%) to obtain 90 mg of a racemate, which was separated by SFC to obtain two isomers, i.e., compound 94-1 (30 mg, retention time 1.80 min, 27%) and compound 94-2 (30 mg, retention time 1.97 min, 27%).

Separation conditions of preparative chromatography: 1. instrument: Waters 150 Prep-SFC E; 2. chromatographic column: Chiralcel OJ column; 3. mobile phase system: A for CO₂; B for 0.1% NH₃·H₂O in MEOH; 4. gradient: B 35%; and 5. flow rate: 70 mL/min.

### Compound 94-1:

¹H NMR (400 MHz, Chloroform-d) δ 7.95 (d, 1H), 7.03 (d, 1H), 5.51 (s, 1H), 4.90-4.80 (m, 2H), 4.66-4.60 (m, 1H), 4.38-4.32 (m, 3H), 4.03-3.98 (m, 1H), 3.63-3.53 (m, 1H), 3.49-3.36 (m, 2H), 3.17-3.08 (m, 1H), 3.07-2.95 (m, 3H), 2.92-2.85 (m, 1H), 2.83-2.76 (m, 1H), 1.50 (d, 6H).

LC-MS (ESI): m/z=498.6[M+H]⁺

### Compound 94-2:

¹H NMR (400 MHz, Chloroform-d) δ 7.95 (d, 1H), 7.03 (d, 1H), 5.49 (s, 1H), 4.89-4.79 (m, 2H), 4.66-4.59 (m, 1H), 4.41-4.35 (m, 3H), 4.04-3.97 (m, 1H), 3.63-3.53 (m, 1H), 3.49-3.37 (m, 2H), 3.17-3.09 (m, 1H), 3.06-2.95 (m, 3H), 2.92-2.84 (m, 1H), 2.83-2.76 (m, 1H), 1.50 (d, 6H).

LC-MS (ESI): m/z=498.6[M+H]⁺

### Example 95

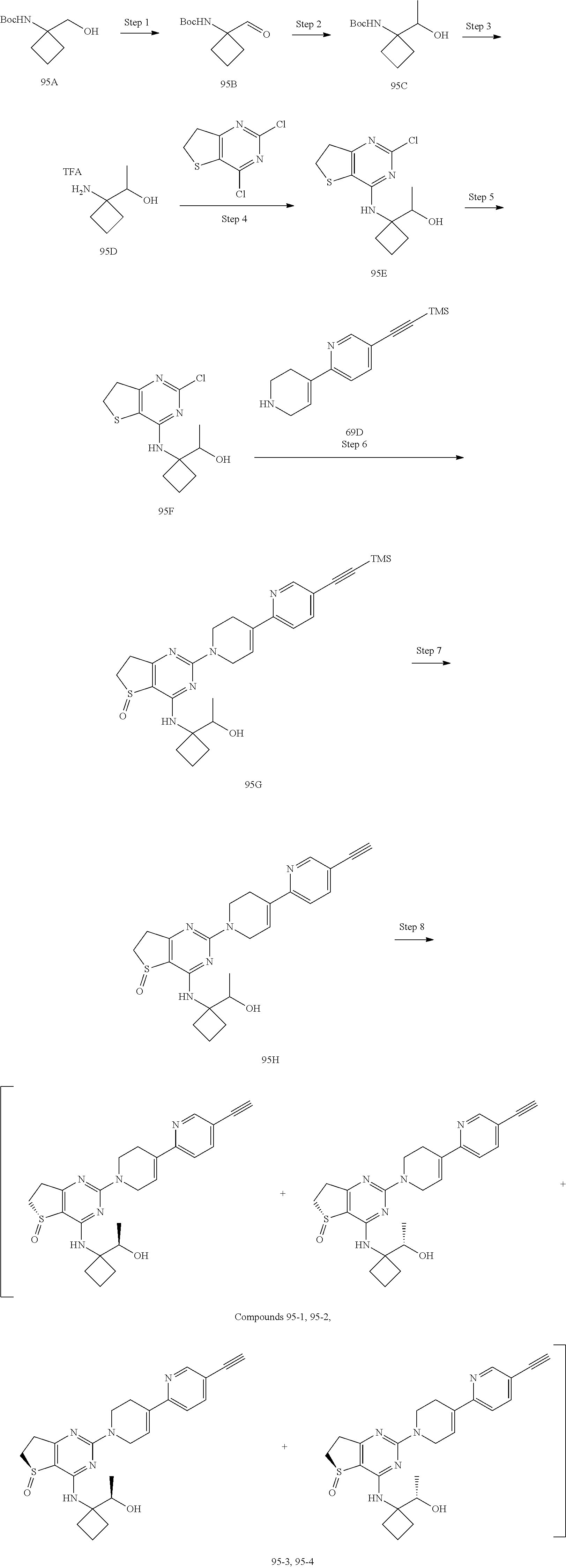

Step 1: Compound 95A (10.0 g, 50.0 mmol) was dissolved in ethyl acetate (50.0 mL), 2-iodoxybenzoic acid (21.0 g, 75.0 mmol) was then added, and the mixture was heated to reflux and reacted overnight. The system was cooled to room temperature, filtered, and concentrated to obtain compound 95B (10.0 g, 95%).

LC-MS (ESI): m/z=200.1 [M+H]⁺

Step 2: Compound 95B (10.0 g, 50.0 mmol) was dissolved in tetrahydrofuran (50.0 mL), methylmagnesium bromide (3.0 M in THF, 100.0 mmol) was then added in an ice bath, and the mixture was slowly heated to room temperature and reacted for 4 hours. After quenching by adding saturated ammonium chloride, the system was extracted with ethyl acetate, dried, and concentrated to obtain compound 95C (10.0 g, 92%).

LC-MS (ESI): m/z=160.1 [M+H-56]⁺

Step 3: Compound 95C (10.0 g, 46.0 mmol) was dissolved in dichloromethane (50.0 mL), and trifluoroacetic acid (20.0 mL) was then added and reacted at room temperature for 2 hours. The system was then concentrated and subjected to column chromatography (dichloromethane/methanol=10/1) to obtain compound 95D (5.6 g, 56%).

LC-MS (ESI): m/z=116.1 [M+H]⁺

Step 4: Compound 95D (5.6 g, 26.0 mmol) was dissolved in acetonitrile (40.0 mL), 2,4-dichloro-6,7-dihydrothieno[3,2-D]pyrimidine (5.1 g, 25.0 mmol), and triethylamine (10.4 g, 104.0 mmol) were then added, heated to 75° C., and reacted for 12 h. The system was then cooled to room temperature, concentrated, and subjected to column chromatography (dichloromethane/methanol=10/1) to obtain compound 95E (3.6 g, 48%).

LC-MS (ESI): m/z=286.1 [M+H]⁺

Step 5: Compound 95E (3.6 g, 12.6 mmol), (S)-(−)-1,1′-bi-2-naphthol (360.0 mg, 1.3 mmol), titanium tetraisopropoxide (180.0 mg, 0.62 mmol), and water (229.0 mg, 12.1 mmol) were dissolved in dichloromethane (80.0 mL). After nitrogen displacement three times, the mixture was stirred at room temperature for 1 hour, tert-butyl hydroperoxide (1.2 g, 13.9 mmol) was added, stirred for 1.5 hours, then concentrated, and subjected to column chromatography (dichloromethane/methanol=10/1) to obtain compound 95F (1.2 g, 31%).

LC-MS (ESI): m/z=302.1 [M+H]⁺

Step 6: Compound 95F (0.7 g, 2.3 mmol) was dissolved in 1,4-dioxane (10.0 mL), and 69D (640.0 mg, 2.5 mmol) and diisopropylethylamine (0.93 g, 7.0 mmol) were then added, heated to 90° C. and reacted for 12 h. The system was then cooled to room temperature, concentrated, and subjected to column chromatography (dichloromethane/methanol=10/1) to obtain compound 95G (1.0 g, 81%).

LC-MS (ESI): m/z=522.1 [M+H]⁺

Step 7: Compound 95G (1.0 g, 1.9 mmol) was dissolved in methanol (10.0 mL), potassium carbonate (276.0 mg, 2.0 mmol) was then added, and the mixture was reacted at room temperature for 2 h, then concentrated, and subjected to column chromatography (dichloromethane/methanol=10/1) to obtain compound 95H (0.7 g, 70%).

LC-MS (ESI): m/z=450.1 [M+H]⁺

Step 8: Compound 95H (1.1 g, 2.3 mmol) was subjected to chiral resolution.

Preparation method: instrument: SHIMADZU LC-20AP, column: Phenomenex C18, mobile phase: (A was 10 mmol NH₄HCO₃ in H₂O; B was acetonitrile); gradient: 28-58% mobile phase B, isocratic elution; flow rate: 25 mL/min, column temperature: 25° C., wavelength: 254 nm, sample preparation: sample concentration 60 mg/ml, acetonitrile/aqueous solution injection: 1.5 ml each time. After separation, the fraction was dried by a rotary evaporator at a bath temperature of 35° C. to obtain compounds 95-1 (retention time: 1.328 minutes), 95-2 (retention time: 1.120 minutes), 95-3 (retention time: 1.388 minutes), and 95-4 (retention time: 1.421 minutes).

### Compound 95-1:

¹H NMR (400 MHz, DMSO-d6) δ 8.64 (d, 1H), 7.89-7.86 (m, 1H), 7.61 (d, 1H), 7.26 (d, 1H), 6.88 (s, 1H), 4.61-4.36 (m, 5H), 4.02 (s, 2H), 3.52-3.35 (m, 1H), 3.29-3.19 (m, 1H), 3.00-2.94 (m, 1H), 2.90-2.85 (m, 1H), 2.63 (s, 2H), 2.21-2.02 (m, 1H), 1.74-1.57 (m, 5H), 1.09 (s, 3H).

LC-MS (ESI): m/z=450.1 [M+H]⁺.

### Compound 95-2:

¹H NMR (400 MHz, DMSO-d6) δ 8.63 (d, 1H), 7.88-7.86 (m, 1H), 7.61 (d, 1H), 7.33 (s, 1H), 6.87 (s, 1H), 4.92 (d, 1H), 4.43 (s, 3H), 4.25-4.16 (m, 1H), 4.04-3.89 (m, 2H), 3.53-3.35 (m, 1H), 2.98-2.84 (m, 2H), 2.64 (d, 2H), 2.47-2.16 (m, 5H), 1.88-1.65 (m, 2H), 1.01 (d, 3H).

LC-MS (ESI): m/z=450.1 [M+H]⁺

### Compound 95-3:

¹H NMR (400 MHz, DMSO-d6) δ 8.64 (d, 1H), 7.89-7.86 (m, 1H), 7.61 (d, 1H), 6.89 (s, 1H), 6.43 (d, 1H), 4.83 (s, 1H), 4.44 (d, 3H), 4.13-4.07 (m, 1H), 4.01 (t, 2H), 3.48-3.33 (m, 2H), 3.04-2.81 (m, 2H), 2.65 (d, 2H), 2.20-2.04 (m, 1H), 1.82-1.66 (m, 3H), 1.66-1.49 (m, 2H), 1.21 (s, 3H).

LC-MS (ESI): m/z=450.1 [M+H]⁺

### Compound 95-4:

¹H NMR (400 MHz, DMSO-d6) δ 8.64 (d, 1H), 7.89-7.87 (m, 1H), 7.76 (s, 1H), 7.62 (d, 1H), 6.88 (s, 1H), 4.51-4.35 (m, 3H), 4.23-4.19 (m, 2H), 4.06-3.88 (m, 3H), 3.56-3.37 (m, 1H), 3.37-3.21 (m, 1H), 3.08-3.02 (m, 1H), 2.94-2.89 (m, 1H), 2.65 (s, 2H), 2.42-2.30 (m, 3H), 1.87-1.64 (m, 2H), 1.01 (d, 3H).

LC-MS (ESI): m/z=450.1 [M+H]⁺

### Example 96

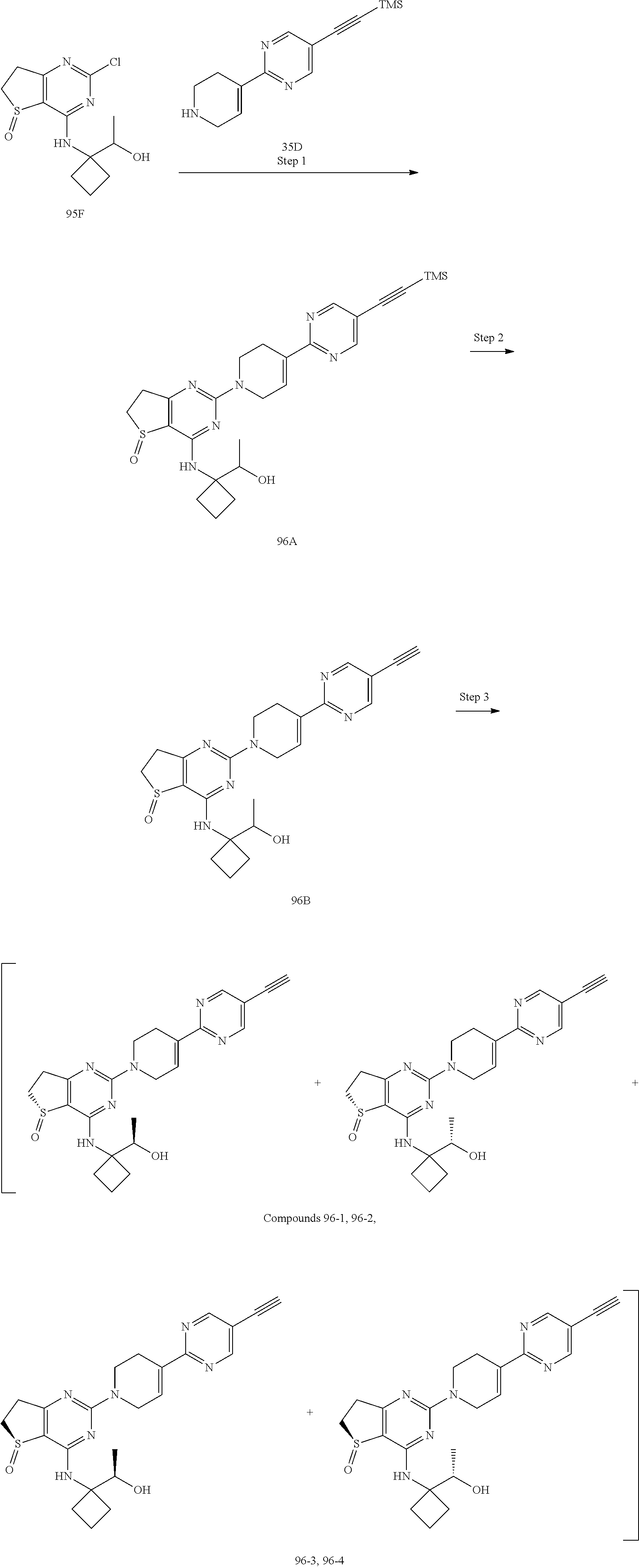

Step 1: Compound 95F (1.2 g, 4.0 mmol) was dissolved in 1,4-dioxane (20.0 mL), and 35D (1.2 g, 4.5 mmol) and diisopropylethylamine (1.6 g, 12.0 mmol) were then added, heated to 90° C. and reacted for 12 h. The system was then cooled to room temperature, concentrated, and subjected to column chromatography (dichloromethane/methanol=10/1) to obtain compound 96A (1.2 g, 57%).

LC-MS (ESI): m/z=523.1 [M+H]⁺

Step 2: Compound 96A (1.2 g, 2.3 mmol) was dissolved in methanol (10.0 mL), potassium carbonate (276.0 mg, 2.0 mmol) was then added, and the mixture was reacted at room temperature for 2 h, then concentrated, and subjected to column chromatography (dichloromethane/methanol=10/1) to obtain compound 96B (1.1 g, 100%).

LC-MS (ESI): m/z=451.1 [M+H]⁺

Step 3: Compound 96B (1.1 g, 2.3 mmol) was subjected to chiral resolution.

Preparation method: instrument: SHIMADZU LC-20AP, column: Phenomenex C18, mobile phase: (phase A: 0.1% TFA in H₂O, phase B: ACN); gradient: 15-45% mobile phase B, isocratic elution; flow rate: 25 mL/min, column temperature: 25° C., wavelength: 254 nm, sample preparation: sample concentration 50 mg/ml, acetonitrile/aqueous solution injection: 1.5 ml each time. After separation, the fraction was dried by a rotary evaporator at a bath temperature of 35° C. to obtain compounds 96-1 (retention time: 1.055 minutes), 96-2 (retention time: 1.040 minutes), 96-3 (retention time: 1.107 minutes), and 96-4 (retention time: 2.457 minutes).

### Compound 96-1:

¹H NMR (400 MHz, DMSO-d6) δ 8.91 (s, 2H), 7.60 (s, 1H), 7.33 (s, 1H), 4.69 (s, 1H), 4.50 (t, 2H), 4.24-4.20 (m, 1H), 4.07-3.99 (m, 2H), 3.96-3.91 (m, 2H), 3.47-3.41 (m, 1H), 3.32-3.19 (m, 1H), 3.04-2.98 (m, 1H), 2.92-2.87 (m, 1H), 2.67 (s, 2H), 2.42-2.29 (m, 3H), 1.79-1.72 (m, 2H), 1.00 (d, 3H).

LC-MS (ESI): m/z=451.1 [M+H]⁺

### Compound 96-2:

¹H NMR (400 MHz, DMSO-d6) δ 8.91 (s, 2H), 7.57 (s, 1H), 7.33 (s, 1H), 4.69 (s, 1H), 4.50 (t, 2H), 4.24-4.20 (m, 1H), 4.07-3.99 (m, 2H), 3.97-3.92 (m, 2H), 3.47-3.41 (m, 1H), 3.32-3.19 (m, 2H), 3.04-2.98 (m, 1H), 2.91-2.86 (m, 1H), 2.67 (s, 2H), 2.42-2.29 (m, 3H), 1.79-1.72 (m, 2H), 1.01 (d, 3H).

LC-MS (ESI): m/z=451.1 [M+H]⁺

### Compound 96-3:

¹H NMR (400 MHz, DMSO-d6) δ 8.91 (s, 2H), 7.34 (s, 1H), 6.55 (s, 1H), 4.69 (s, 1H), 4.51 (d, 2H), 4.16 (d, 1H), 4.01 (t, 2H), 3.50-3.41 (m, 3H), 3.12-2.83 (m, 2H), 2.69 (s, 2H), 2.22-2.01 (m, 1H), 1.75-1.69 (m, 3H), 1.57 (t, 2H), 1.20 (s, 3H).

LC-MS (ESI): m/z=451.1 [M+H]⁺

### Compound 96-4:

¹H NMR (400 MHz, DMSO-d6) δ 8.91 (s, 2H), 7.34 (s, 1H), 6.55 (s, 1H), 4.69 (s, 1H), 4.51 (d, 3H), 4.16 (d, 1H), 4.01 (t, 2H), 3.51-3.42 (m, 2H), 3.12-2.83 (m, 2H), 2.69 (s, 2H), 2.21-2.00 (m, 1H), 1.75-1.69 (m, 3H), 1.57 (t, 2H), 1.21 (s, 3H).

LC-MS (ESI): m/z=451.1 [M+H]⁺

### Example 97

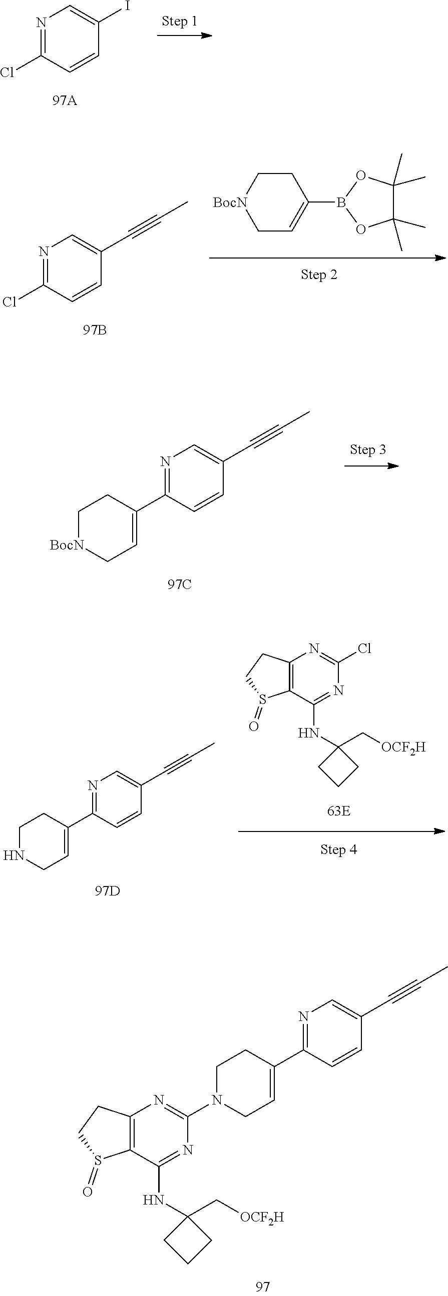

Step 1: Compound 97A (2.0 g, 8.4 mmol) was dissolved in toluene (50 mL), and tributylpropynylstannane (3.1 g, 9.2 mmol) and tetrakis(triphenylphosphine)palladium (0.1 g, 0.8 mmol) were successively added. After nitrogen displacement three times, the system was heated to 100° C. and reacted for 14 h. After the reaction was complete as monitored by a TLC plate, the system was concentrated and purified by a silica gel column (PE/EA=10/1) to obtain the product 97B (1.2 g, 94.4%).

¹H NMR (400 MHz, CDCl₃) δ 8.37 (s, 1H), 7.62 (d, 1H), 7.25 (d, 1H), 2.07 (s, 3H).

Step 2: Compound 97B (1.2 g, 7.9 mmol) was dissolved in 1,4-dioxane (20 mL), and 1,1-bis(diphenylphosphine)ferrocenepalladium dichloride (0.6 g, 0.8 mmol), cesium carbonate (5.2 g, 15.8 mmol), N-Boc-1,2,5,6-tetrahydropyridine-4-boronic acid pinacol ester (2.9 g, 9.5 mmol), and water (4 mL) were then added. After nitrogen displacement three times, the mixture was heated to 90° C. and reacted for 4 h, and the system was then passed through a short silica gel column, eluted with ethyl acetate, concentrated, and subjected to column chromatography (petroleum ether/ethyl acetate=9/1) to obtain compound 97C (2.0 g, 84.6%).

LC-MS (ESI): m/z=299.4 [M+H]⁺

Step 3: Compound 97C (1.5 g, 5.0 mmol) was dissolved in dichloromethane (10 mL), trifluoroacetic acid (3.8 mL) was then added, and the mixture was reacted at room temperature for half an hour and then concentrated to obtain compound 97D (0.9 g, 90.3%), which was directly used for the next step.

LC-MS (ESI): m/z=199.4 [M+H]⁺

Step 4: Compound 63E (1.1 g, 3.2 mmol) was dissolved in 1,4-dioxane (20.0 mL), and compound 97D (0.7 g, 3.5 mmol) and diisopropylethylamine (2.1 g, 16.0 mmol) were then added, heated to 90° C., and reacted for 12 h. The system was then cooled to room temperature, concentrated, and subjected to column chromatography (dichloromethane/methanol=10/1) to obtain the target product, which was further subjected to chiral resolution to obtain compound 97 (20 mg, 2%).

Preparation method: instrument: Waters 150 Prep-SFC, column: Chiralcel OX Colum, mobile phase: (phase A: CO2, phase B: 0.1% NH₃·H₂O in MEOH and ACN); gradient: 40% mobile phase B, isocratic elution; flow rate: 120 mL/min, back pressure: 100 bar, column temperature: 25° C., wavelength: 220 nm, cycle time: 4.9 min, sample preparation: sample concentration 50 mg/ml, acetonitrile solution injection: 2 ml each time. After separation, the fraction was dried on a rotary evaporator at a bath temperature of 35° C. to obtain compound 97 (retention time: 0.952 minutes).

¹H NMR (400 MHz, DMSO-d6) δ 8.55 (d, 1H), 7.88 (s, 1H), 7.78-7.76 (m, 1H), 7.56 (d, 1H), 6.90-6.52 (m, 2H), 4.39 (s, 2H), 4.32-4.20 (m, 2H), 3.96 (s, 2H), 3.52-3.39 (m, 1H), 3.24-3.16 (m, 1H), 3.00-2.85 (m, 2H), 2.61 (s, 2H), 2.44-2.15 (m, 4H), 2.08 (s, 3H), 1.97-1.77 (m, 2H).

LC-MS (ESI): m/z=500.1 [M+H]⁺.

### Example 98

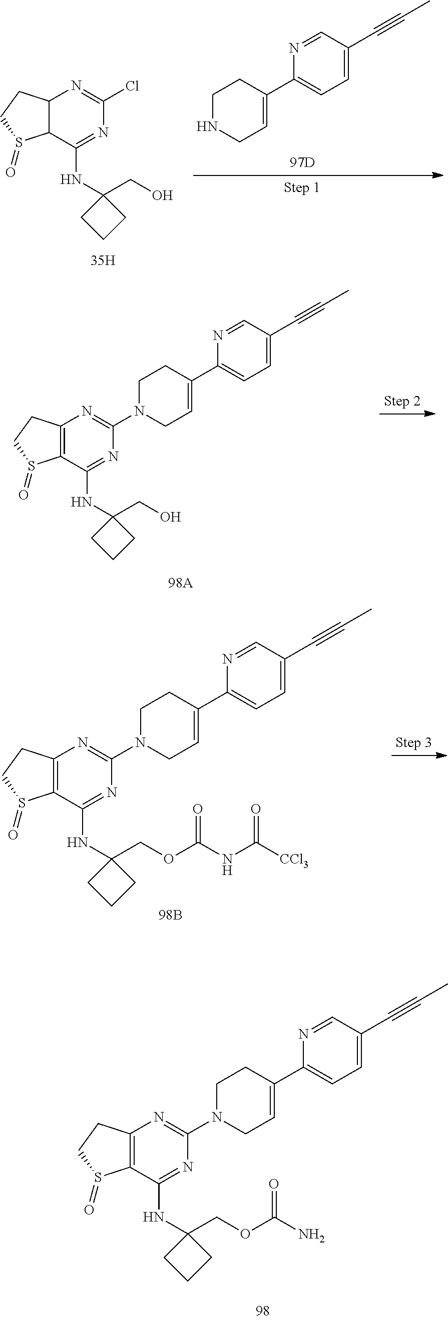

Step 1: Compound 35H (1.68 g, 5.8 mmol) was dissolved in 1,4-dioxane (20.0 mL), and compound 97D (1.4 g, 7.0 mmol) and diisopropylethylamine (3.0 g, 23.3 mmol) were then added, heated back to 90° C., and reacted for 12 h. The system was then cooled to room temperature, concentrated, and subjected to column chromatography (dichloromethane/methanol=10/1) to obtain the target product, which was further subjected to chiral resolution to obtain compound 98A (2.5 g, 95%).

LC-MS (ESI): m/z=450.1 [M+H]⁺

Step 2: Compound 98A (2.4 g, 5.3 mmol) was dissolved in dichloromethane (10.0 mL), and trichloroacetyl isocyanate (1.3 g, 6.9 mmol) was added in an ice bath and stirred in the ice bath for one hour. The reaction liquid was concentrated to obtain compound 98B (2.5 g, 73%).

LC-MS (ESI): m/z=637.1 [M+H]⁺

Step 3: Compound 98B (2.5 g, 3.9 mmol) was dissolved in methanol (20 mL), and potassium carbonate (2.2 g, 15.7 mmol) and water (20 mL) were added in an ice bath and stirred at room temperature 2.5 hours. The reaction liquid was diluted by adding water and extracted with dichloromethane, and the organic phases were combined, dried, filtered, and concentrated to obtain compound 98 (0.4 g, 20%).

¹H NMR (400 MHz, DMSO-d6) δ 8.55 (d, 1H), 7.78-7.75 (m, 1H), 7.66 (s, 1H), 7.56 (d, 1H), 6.84 (s, 1H), 6.51 (s, 2H), 4.38 (d, 4H), 3.97 (t, 2H), 3.46-3.40 (m, 1H), 3.24-3.17 (m, 1H), 2.99-2.85 (m, 2H), 2.61 (s, 2H), 2.43-2.21 (m, 4H), 2.08 (s, 3H), 1.87-1.81 (m, 2H).

LC-MS (ESI): m/z=493.1 [M+H]⁺

### Example 99

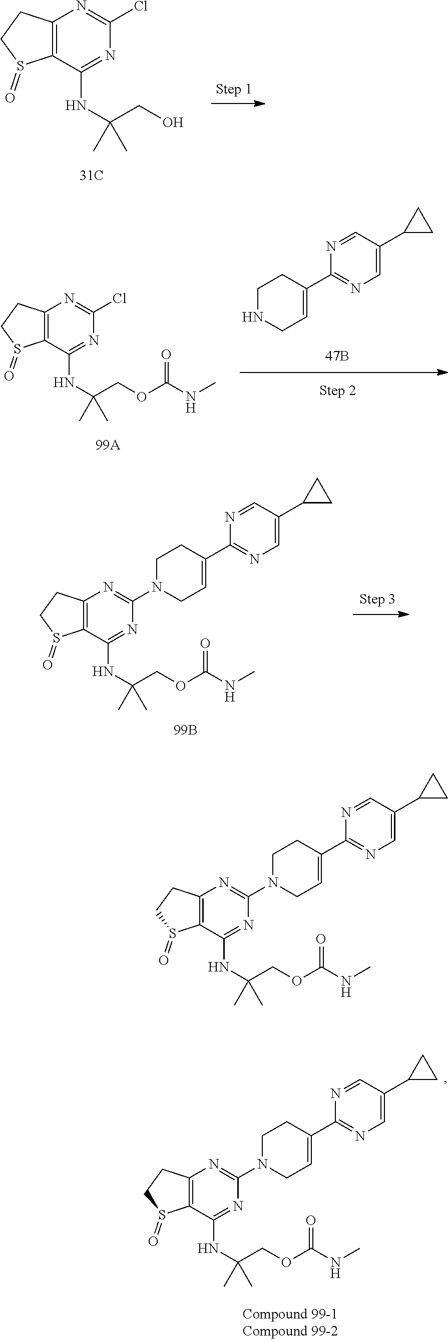 [SMILES: CC(C)(CO)Nc1nc(Cl)nc2c1S(=O)CC2 ; CNC(=O)OCC(C)(C)Nc1nc(Cl)nc2c1S(=O)CC2 ; C1=C(c2ncc(C3CC3)cn2)CCNC1 ; CNC(=O)OCC(C)(C)Nc1nc(N2CC=C(c3ncc(C4CC4)cn3)CC2)nc2c1S(=O)CC2 ; CNC(=O)OCC(C)(C)Nc1nc(N2CC=C(c3ncc(C4CC4)cn3)CC2)nc2c1[S@](=O)CC2 ; CNC(=O)OCC(C)(C)Nc1nc(N2CC=C(c3ncc(C4CC4)cn3)CC2)nc2c1[S@@](=O)CC2]

Step 1: Compound 31C (2.5 g, 9.1 mmol) was dissolved in N,N-dimethylformamide (30 mL), and methylaminoformyl chloride (1.1 g, 11.0 mmol) and triethylamine (1.5 g, 13.6 mmol) were added and stirred at 65° C. overnight under nitrogen protection. The reaction liquid was concentrated and subjected to silica gel column chromatography (dichloromethane/methanol (v/v)=95/5) to obtain compound 99A (1.1 g, 36.4%).

LC-MS (ESI): m/z=333.8 [M+H]⁺

Step 2: Compound 99A (1.0 g, 3.0 mmol) was dissolved in 1,4-dioxane (30 mL), and compound 47B (660 mg, 3.3 mmol) and N,N-diisopropylethylamine (1.2 g, 9.0 mmol) were added and stirred at 90° C. overnight under nitrogen protection. After the reaction was complete, the reaction liquid was concentrated and subjected to silica gel column chromatography (dichloromethane/methanol (v/v)=95/5) to obtain compound 99B (1.2 g, 78.5%).

LC-MS (ESI): m/z=498.6 [M+H]⁺

Step 3: Compound 101B (1.3 mg) was subjected to chiral resolution to obtain compound 101 (208 mg).

Preparation method: instrument: Waters 150 Prep-SFC E, column: Chiralcel OJ column, mobile phase: (phase A: CO2, phase B: 0.1% NH₃·H₂O in MEOH; gradient: 30% mobile phase B, isocratic elution; flow rate: 120 mL/min, back pressure: 100 bar, column temperature: 25° C., wavelength: 220 nm, cycle time: 4.2 min, sample preparation: sample concentration 10 mg/ml, acetonitrile solution injection: 1.5 ml each time. After separation, the fraction was dried in a water bath at 35° C. by a rotary evaporator to obtain a product. The solvent was dried with a freeze-dryer at −80° C. to obtain a final product of compound 99-1 (retention time: 1.789 minutes) and compound 99-2 (retention time: 2.042 minutes).

¹H NMR (400 MHz, DMSO-d₆) δ 8.54 (s, 2H), 7.19 (s, 1H), 7.09-7.04 (m, 1H), 6.61 (s, 1H), 4.46 (s, 2H), 4.35-4.28 (m, 2H), 3.99 (t, 2H), 3.47-3.37 (m, 1H), 3.28-3.21 (m, 1H), 2.99-2.96 (m, 1H), 2.90-2.84 (m, 1H), 2.67 (s, 2H), 2.56 (d, 3H), 1.97-1.90 (m, 1H), 1.45 (d, 6H), 1.06-1.01 (m, 2H), 0.86-0.82 (m, 2H).

¹H NMR (400 MHz, DMSO-d₆) δ 8.54 (s, 2H), 7.19 (s, 1H), 7.09-7.04 (m, 1H), 6.62 (s, 1H), 4.46 (s, 2H), 4.35-4.28 (m, 2H), 3.99 (t, 2H), 3.46-3.38 (m, 1H), 3.28-3.23 (m, 1H), 2.99-2.93 (m, 1H), 2.89-2.84 (m, 1H), 2.67 (s, 2H), 2.56 (d, 3H), 1.97-1.90 (m, 1H), 1.45 (d, 6H), 1.04-1.01 (m, 2H), 0.86-0.82 (m, 2H).

### Example 100

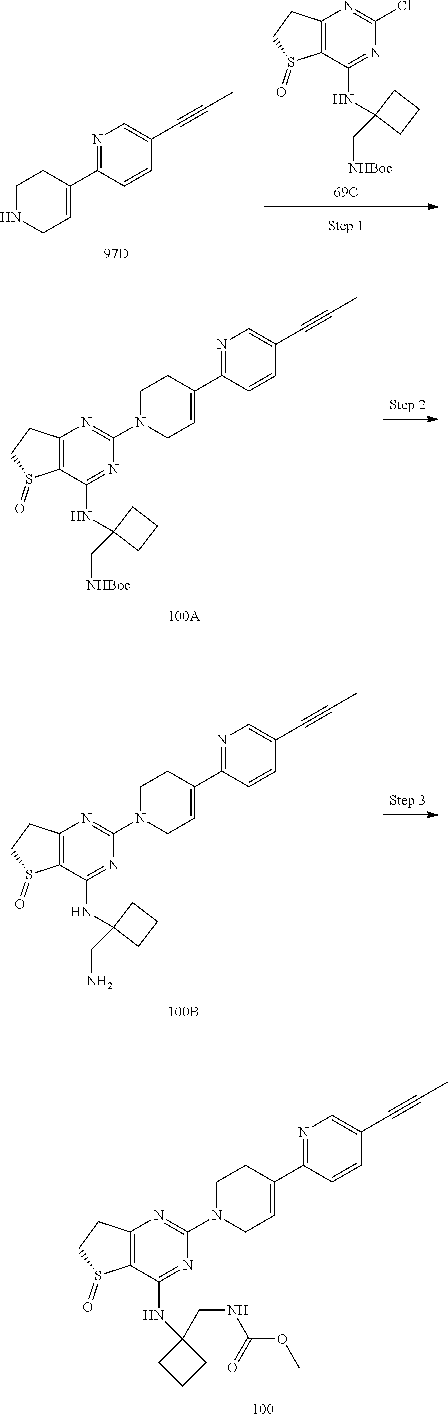 [SMILES: CC#Cc1ccc(C2=CCNCC2)nc1 ; CC(C)(C)OC(=O)NCC1(Nc2nc(Cl)nc3c2[S@](=O)CC3)CCC1 ; CC#Cc1ccc(C2=CCN(c3nc4c(c(NC5(CNC(=O)OC(C)(C)C)CCC5)n3)[S@](=O)CC4)CC2)nc1 ; CC#Cc1ccc(C2=CCN(c3nc4c(c(NC5(CN)CCC5)n3)[S@](=O)CC4)CC2)nc1 ; CC#Cc1ccc(C2=CCN(c3nc4c(c(NC5(CNC(=O)OC)CCC5)n3)[S@](=O)CC4)CC2)nc1]

Step 1: Compound 97D (0.9 g, 4.5 mmol) was dissolved in 1,4-dioxane (30.0 mL), compound 69C (1.5 g, 4.0 mmol) and N,N-diisopropylethylamine (1.2 g, 9 mmol) were then added, and the mixture was heated to 90° C. and reacted for 12 h. The system was then cooled to room temperature, concentrated, and subjected to column chromatography (dichloromethane/methanol=10/1) to obtain compound 100A (2.1 g, 84.2%).

LC-MS (ESI): m/z=549.5 [M+H]⁺

Step 2: Compound 100A (2.1 g, 3.8 mmol) was dissolved in dichloromethane (15 mL), and trifluoroacetic acid (5 mL) was then added and reacted at room temperature for half an hour. The system was then concentrated and subjected to column chromatography (dichloromethane/methanol=10/1) to obtain compound 100B (1.5 g, 88.2%).

LC-MS (ESI): m/z=449.5 [M+H]⁺

Step 3: Compound 100B (1.2 g, 2.7 mmol) was dissolved in dichloromethane (20.0 mL), and methyl chloroformate (0.3 g, 3.0 mmol) and triethylamine (0.8 g, 8.0 mmol) were then added and reacted at room temperature for half an hour. The system was concentrated, and subjected to column chromatography (dichloromethane/methanol=10/1) to obtain compound 100 (1.5 g, 88.2%).

¹H NMR (400 MHz, DMSO-d₆) δ 8.54 (d, 1H), 7.78-7.75 (m, 1H), 7.56 (d, 1H), 7.47 (s, 1H), 7.16 (t, 1H), 6.84 (s, 1H), 4.43 (s, 2H), 3.98 (t, 2H), 3.59-3.45 (m, 5H), 3.43-3.39 (m, 1H), 3.25-3.17 (m, 1H), 2.96-2.84 (m, 2H), 2.61 (s, 2H), 2.39-2.18 (m, 4H), 2.08 (s, 3H), 1.83-1.72 (m, 2H).

LC-MS (ESI): m/z=507.1 [M+H]⁺

### Example 101

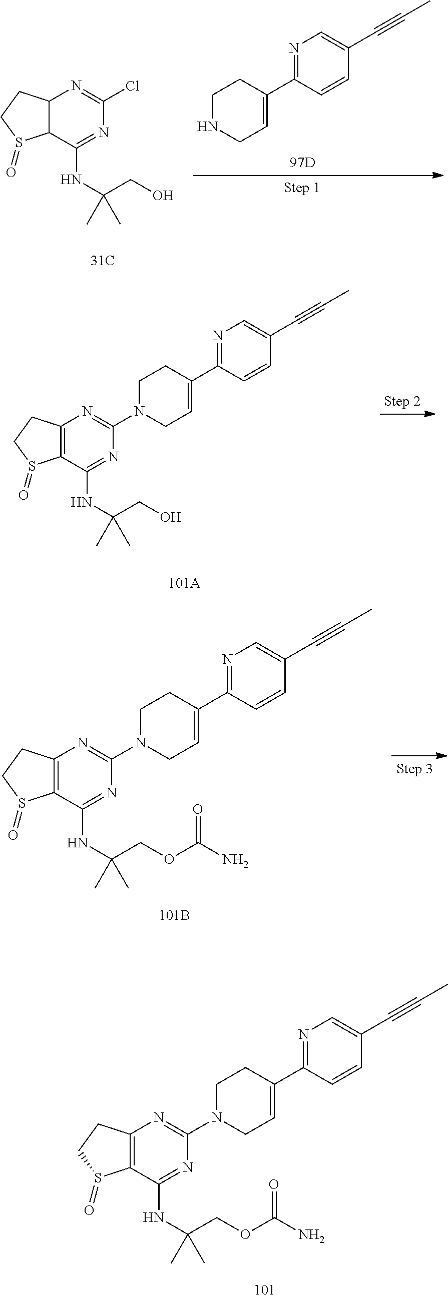 [SMILES: CC(C)(CO)NC1=NC(Cl)=NC2CCS(=O)C12 ; CC#Cc1ccc(C2=CCNCC2)nc1 ; CC#Cc1ccc(C2=CCN(c3nc4c(c(NC(C)(C)CO)n3)S(=O)CC4)CC2)nc1 ; CC#Cc1ccc(C2=CCN(c3nc4c(c(NC(C)(C)COC(N)=O)n3)S(=O)CC4)CC2)nc1 ; CC#Cc1ccc(C2=CCN(c3nc4c(c(NC(C)(C)COC(N)=O)n3)[S@](=O)CC4)CC2)nc1]

Step 1: Compound 31C (1.1 g, 4.0 mmol) was dissolved in 1,4-dioxane (30 mL), and compound 97D (870 mg, 4.4 mmol) and N,N-diisopropylethylamine (1.6 g, 12 mmol) were added and stirred at 90° C. overnight under nitrogen protection. After the reaction was complete, the reaction liquid was cooled to room temperature, then concentrated, and subjected to silica gel column chromatography (dichloromethane/methanol (v/v)=95/5) to obtain compound 101A (1.4 g, 80%).

LC-MS (ESI): m/z=438.6 [M+H]⁺

Step 2: Compound 101A (1.6 g, 3.63 mmol) was dissolved in dichloromethane (5 mL), and trichloroacetyl isocyanate (820 mg, 4.36 mmol) was added in an ice bath and stirred in the ice bath for one hour. After the reaction liquid was concentrated to remove dichloromethane, methanol (20 mL) was added, and potassium carbonate (1.51 g, 10.89 mmol) and water (20 mL) were added in an ice bath and stirred at room temperature for 2.5 hours. The reaction liquid was diluted by adding water and extracted with dichloromethane, and the organic phases were combined, dried, filtered, concentrated, and subjected to silica gel column chromatography (dichloromethane/methanol (v/v)=95/5) to obtain compound 101B (1.3 g, 74%).

LC-MS (ESI): m/z=481.6 [M+H]⁺

Step 3: Compound 101B (1.3 g) was subjected to chiral resolution to obtain compound 101 (208 mg).

Preparation method: instrument: Waters 150 Prep-SFC F, column: Chiralcel OJ Column, mobile phase: (phase A: CO₂, phase B: 0.1% NH₃·H₂O in MEOH; gradient: 35% mobile phase B, isocratic elution; flow rate: 100 mL/min, back pressure: 100 bar, column temperature: 25° C., wavelength: 220 nm, cycle time: 6 min, sample preparation: sample concentration 50 mg/ml, acetonitrile solution injection: 4 ml each time. Retention time: 2.485 minutes. After separation, the fraction was dried in a water bath at 35° C. by a rotary evaporator to obtain a product. The solvent was dried by a lyophilizer at −80° C. to obtain the final product 101.

¹H NMR (400 MHz, DMSO-d₆) δ 8.55 (d, 1H), 7.78-7.76 (m, 1H), 7.57 (d, 1H), 6.85 (d, 1H), 6.69 (t, 1H), 6.55 (s, 2H), 4.44 (s, 2H), 4.29 (s, 2H), 4.00 (t, 2H), 3.41-3.38 (m, 1H), 3.29-3.22 (m, 1H), 2.99-2.96 (m, 1H), 2.90-2.84 (m, 1H), 2.64 (s, 2H), 2.08 (s, 3H), 1.46 (d, 6H).

LC-MS (ESI): m/z=481.6 [M+H]⁺

### Example 102

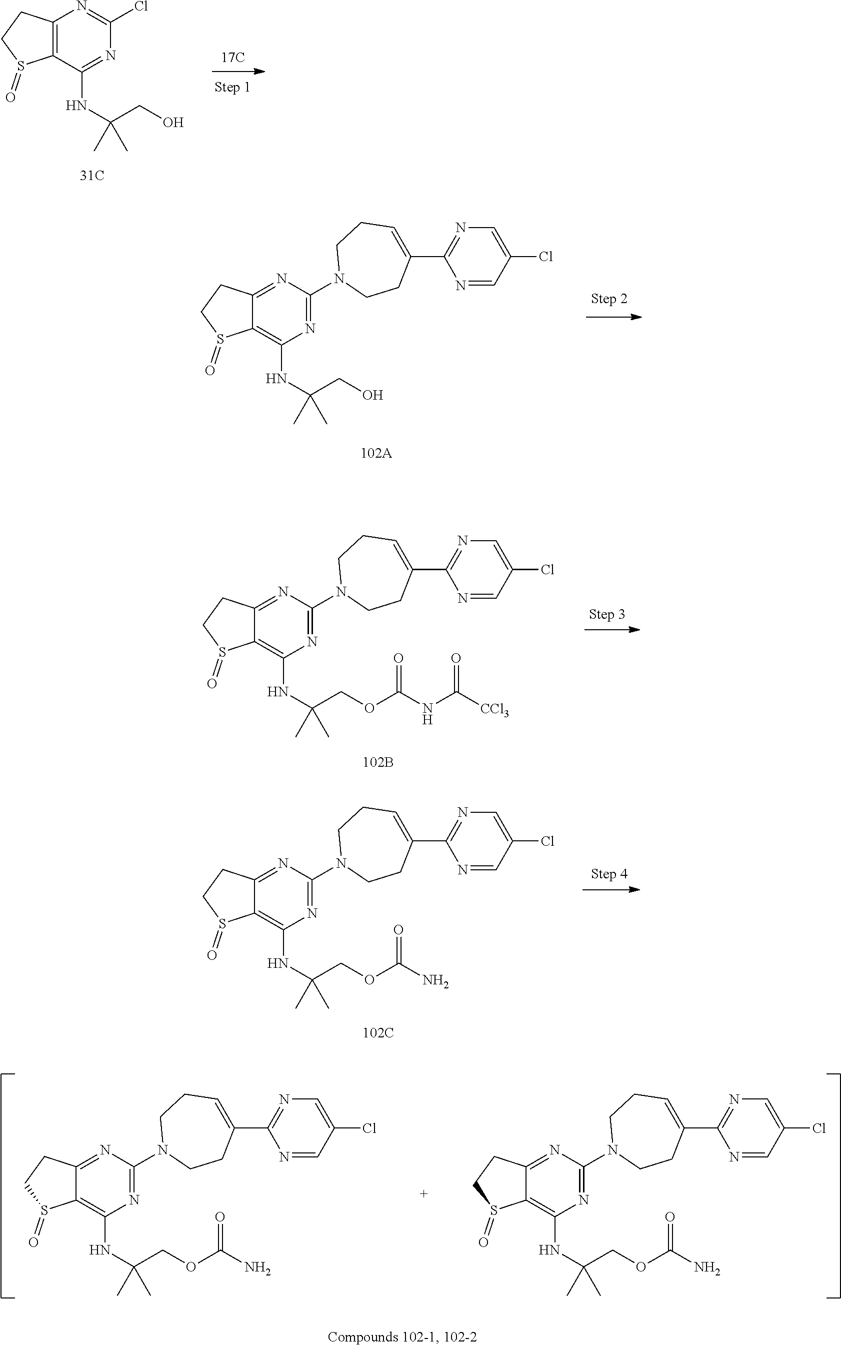

Step 1: 31C (300 mg, 1.09 mmol) was dissolved in 1,4-dioxane (10 mL), and 17C (320 mg, 1.31 mmol) and N,N-diisopropylethylamine (420 mg, 3.26 mmol) were successively added. After the addition was complete, the reaction liquid was heated to 85° C. and stirred for 16 h under nitrogen protection. After the reaction liquid naturally returned to room temperature, the reaction was diluted by adding water (50 mL) and extracted three times with ethyl acetate (40 mL), and the organic phases were combined, dried over anhydrous sodium sulfate, and then concentrated in vacuum to obtain a crude product, which was purified by column chromatography (eluents: DCM:MeOH=100:1 to 10:1) to obtain the target compound 102A (350 mg, 71.66%).

LC-MS (ESI): m/z=449.2 [M+H]⁺

Step 2: Under nitrogen protection, compound 102A (120 mg, 0.27 mmol) was dissolved in dichloromethane (12 mL), and trichloroacetyl isocyanate (60 mg, 0.32 mmol) was slowly dropwise added under stirring in an ice bath. After the addition was complete, the reaction liquid was stirred at 0° C. for 1 h. After the raw material disappeared as monitored by a TLC spotting plate (developing agent: DCM/MeOH=10:1), the reaction liquid was concentrated under reduced pressure to obtain crude product 102B (170 mg), which was directly used in the next reaction.

LC-MS (ESI): m/z=636.0 [M+H]⁺

Step 3: Crude compound 102B (170 mg, 0.27 mmol) was dissolved in methanol (8 mL), and potassium carbonate (110 mg, 0.80 mmol) was added at room temperature. After the addition was complete, the mixture was stirred at room temperature 1.5 h under nitrogen protection. After the reaction was complete, the reaction liquid was concentrated under reduced pressure to obtain a crude product, and the crude product was purified by column chromatography (eluents: DCM:MeOH=100:1 to 9:1), followed by further purification through a reverse phase column (eluents: water:acetonitrile=100:1 to 1:9) to obtain the target compound 102C (120 mg, 91.44%).

LC-MS (ESI): m/z=492.1 [M+H]⁺

Step 4: Compound 102C (120 mg, 0.24 mmol) was subjected to chiral resolution to obtain compound 102-1 (18.4 mg) and compound 102-2 (14.2 mg).

Analysis method: instrument: SHIMADZU LC-30AD, column: Chiralcel Whelk Column; mobile phase: A: CO₂, B: 0.05% DEA in IPA and ACN; gradient: 50% B in A; flow rate: 3 mL/min, column temperature: 35° C., wavelength: 220 nm.

Preparation method: instrument: Waters 150 Prep-SFC F, column: Chiralcel Whelk Column (250 mm×30 mm, 10 μm) mobile phase: A: CO₂, B: 0.1% NH₃·H₂O in IPA and ACN; gradient: 55% B gradient elution, flow rate: 60 mL/min, column temperature: 25° C., wavelength: 220 nm, cycle time: 4.2 min, sample preparation: sample concentration 5 mg/ml, ethanol solution injection: 1 ml each time. After separation, the fraction was dried by a rotary evaporator at a bath temperature of 35° C. to obtain compound 102-1 and compound 102-2.

### Compound 102-1:

¹H NMR (400 MHz, CDCl₃) δ 8.53 (s, 2H), 7.33-7.29 (m, 1H), 5.30 (s, 1H), 4.32-4.23 (m, 2H), 3.99-3.88 (m, 4H), 3.56-3.48 (m, 1H), 3.33-3.26 (m, 1H), 3.08-3.02 (m, 2H), 2.96-2.83 (m, 3H), 2.61-2.56 (m, 1H), 1.67-1.59 (m, 2H), 1.45-1.43 (m, 6H).

LC-MS (ESI): m/z=492.1 [M+H]⁺

### Compound 102-2:

¹H NMR (400 MHz, CDCl₃) δ 8.53 (s, 2H), 7.33-7.29 (m, 1H), 5.30 (s, 1H), 4.32-4.23 (m, 2H), 4.03-3.88 (m, 4H), 3.56-3.48 (m, 1H), 3.33-3.26 (m, 1H), 3.08-3.02 (m, 2H), 2.96-2.83 (m, 3H), 2.63-2.56 (m, 1H), 1.67-1.59 (m, 2H), 1.45-1.43 (m, 6H).

LC-MS (ESI): m/z=492.1 [M+H]⁺

### Example 103

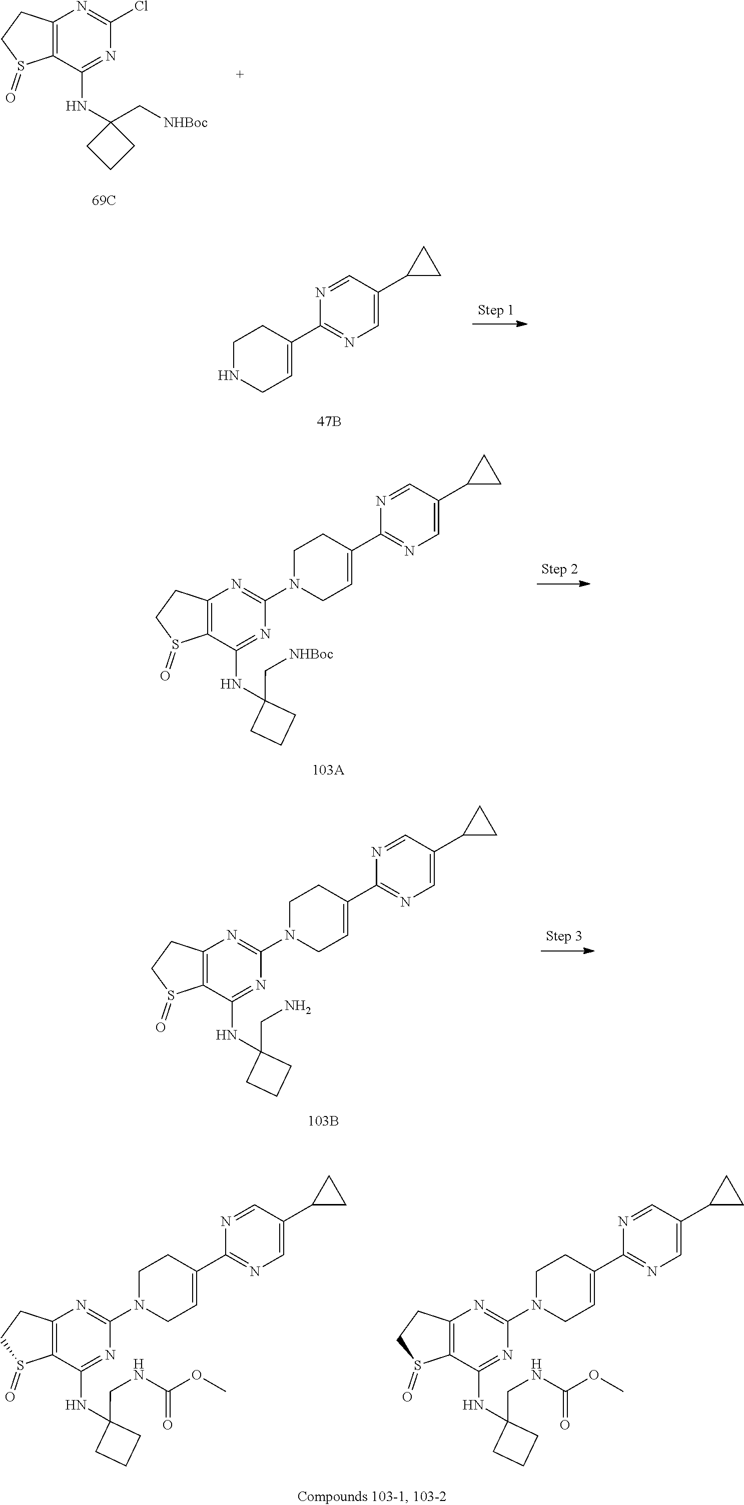 [SMILES: CC(C)(C)OC(=O)NCC1(Nc2nc(Cl)nc3c2S(=O)CC3)CCC1 ; C1=C(c2ncc(C3CC3)cn2)CCNC1 ; CC(C)(C)OC(=O)NCC1(Nc2nc(N3CC=C(c4ncc(C5CC5)cn4)CC3)nc3c2S(=O)CC3)CCC1 ; NCC1(Nc2nc(N3CC=C(c4ncc(C5CC5)cn4)CC3)nc3c2S(=O)CC3)CCC1 ; COC(=O)NCC1(Nc2nc(N3CC=C(c4ncc(C5CC5)cn4)CC3)nc3c2[S@](=O)CC3)CCC1 ; COC(=O)NCC1(Nc2nc(N3CC=C(c4ncc(C5CC5)cn4)CC3)nc3c2[S@@](=O)CC3)CCC1]

Step 1: Intermediate 69C (450 mg, 1.16 mmol), intermediate 47B (280 mg, 1.39 mmol), and N,N-diisopropylethylamine (748 mg, 5.80 mmol) were suspended in 1,4-dioxane (5 mL) and reacted at 80° C. for 16 h under nitrogen protection, and the reaction liquid was cooled to room temperature and then dripped into 10 mL of water. The system was extracted twice with 10 mL of dichloromethane, and the organic phases were combined, then dried, concentrated, and separated by silica gel column chromatography (DCM:MeOH=15:1) to obtain compound 103A (600 mg, 94%).

LCMS(ESI): m/z=552.2 [M+H]⁺

Step 2: Compound 103A (600 mg, 1.08 mmol) was dissolved in 4 mL of a solution of hydrogen chloride dioxane (4 M) and stirred at room temperature for 3 h. After the reaction was complete as monitored by LCMS, the system was concentrated under reduced pressure and dried, and crude 103B (580 mg) was directly used for the next step.

LCMS (ESI): m/z=452.3 [M+H]⁺

Step 3: Crude 103B (580 mg, 1.08 mmol calculated based on the 100% yield of the second step) was dissolved in 20 mL of dichloromethane, and triethylamine (218 mg, 2.16 mmol) and methyl chloroformate (124 mg, 1.30 mmol) were added at 0° C. under stirring and reacted in an ice bath for 1 h. After the reaction was complete as monitored by LCMS, 5 mL of water was added, and the mixture was extracted with dichloromethane (10 mL×2). The organic phases were combined, dried over anhydrous sodium sulfate, and concentrated under reduced pressure. After concentration, the system was separated by silica gel column chromatography (DCM:MeOH=20:1), followed by SFC separation to obtain two optically pure isomers 103-1 (17.2 mg, 3%, with absolute configuration unknown) and 103-2 (108.2 mg, 20%, with absolute configuration unknown).

### Chiral Separation Method:

instrument: Instrument: Waters 150 Prep-SFC F, chiral column: Chiralcel Whelk-Column mobile phase: A for CO₂; B for 0.1% NH₃·H₂O in MEOH and CAN, gradient: B 50%; flow rate: 100 mL/min, column pressure: 100 bar, column temperature: 25° C., detection wavelength: 220 nm, cycle time: 3.0 min.

### Compound 103-1:

retention time: 0.832 min, ¹H NMR (400 MHz, DMSO-d6) δ 8.54 (s, 2H), 7.49 (s, 1H), 7.19 (s, 2H), 4.44 (s, 2H), 3.97 (t, 2H), 3.61-3.35 (m, 6H), 3.25-3.17 (m, 1H), 3.01-2.83 (m, 2H), 2.65 (s, 2H), 2.42-2.16 (m, 4H), 1.98-1.88 (m, 1H), 1.85-1.75 (m, 2H), 1.06-0.96 (m, 2H), 0.89-0.75 (m, 2H).

LCMS (ESI): m/z=510.2 [M+H]⁺

### Compound 103-2:

retention time: 0.990 min, ¹H NMR (400 MHz, DMSO-d6) δ 8.54 (s, 2H), 7.49 (s, 1H), 7.19 (s, 2H), 4.44 (s, 2H), 3.97 (t, 2H), 3.61-3.35 (m, 6H), 3.25-3.17 (m, 1H), 3.01-2.83 (m, 2H), 2.65 (s, 2H), 2.42-2.16 (m, 4H), 1.98-1.88 (m, 1H), 1.85-1.75 (m, 2H), 1.06-0.96 (m, 2H), 0.89-0.75 (m, 2H).

LCMS (ESI): m/z=510.2 [M+H]⁺

### Example 104

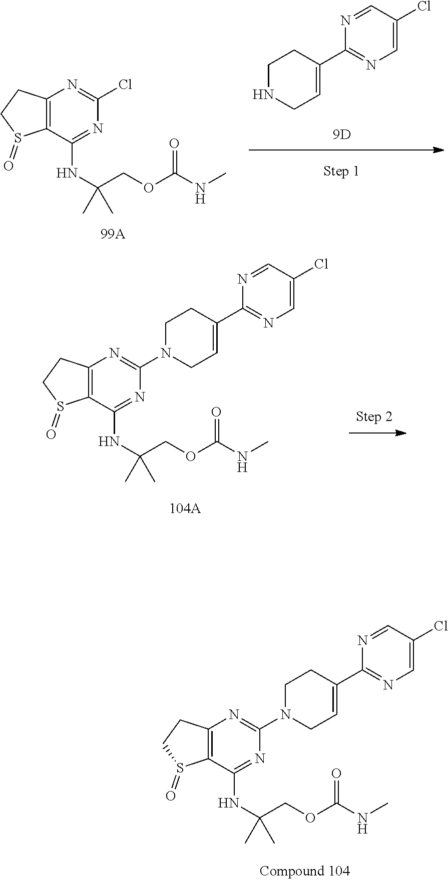 [SMILES: CNC(=O)OCC(C)(C)Nc1nc(Cl)nc2c1S(=O)CC2 ; Clc1cnc(C2=CCNCC2)nc1 ; CNC(=O)OCC(C)(C)Nc1nc(N2CC=C(c3ncc(Cl)cn3)CC2)nc2c1S(=O)CC2 ; CNC(=O)OCC(C)(C)Nc1nc(N2CC=C(c3ncc(Cl)cn3)CC2)nc2c1[S@](=O)CC2]

Step 1: Compound 99A (600 mg, 1.8 mmol) was dissolved in 1,4-dioxane (20 mL), and compound 9D (420 mg, 2.16 mmol) and N,N-diisopropylethylamine (1.4 g, 10.8 mmol) were added and stirred at 90° C. overnight under nitrogen protection. After the reaction was complete, the reaction liquid was concentrated and subjected to silica gel column chromatography (dichloromethane/methanol (v/v)=95/5) to obtain compound 104A (500 mg, 56.5%).

LC-MS (ESI): m/z=492.1 [M+H]⁺

Step 2: Compound 104A (500 mg) was subjected to chiral resolution to obtain compound 104 (282 mg, 56.4%).

Preparation method: instrument: Waters 150 Prep-SFC F, column: Chiralcel Whelk-Column, mobile phase: (phase A: CO₂, phase B: 0.1% NH₃·H₂O in IPA and ACN; gradient: 45% mobile phase B, isocratic elution; flow rate: 100 mL/min, back pressure: 100 bar, column temperature: 25° C., wavelength: 220 nm, cycle time: 3.1 min, sample preparation: sample concentration 10 mg/ml, acetonitrile solution injection: 5.0 ml each time. After separation, the fraction was dried in a water bath at 35° C. by a rotary evaporator to obtain a product. The solvent was dried with a freeze-dryer at −80° C. to obtain a final product of compound 104 (retention time: 0.867 minutes).

LC-MS (ESI): m/z=492.1 [M+H]⁺

¹H NMR (400 MHz, DMSO-d₆) δ 8.90 (s, 2H), 7.30 (d, 1H), 7.10 (d, 1H), 6.69 (s, 1H), 4.49 (s, 2H), 4.41-4.26 (m, 2H), 4.00 (t, 2H), 3.50-3.39 (m, 1H), 3.32-3.21 (m, 1H), 3.02-2.92 (m, 1H), 2.92-2.82 (m, 1H), 2.67 (s, 2H), 2.56 (d, 3H), 1.45 (d, 6H).

### Example 105

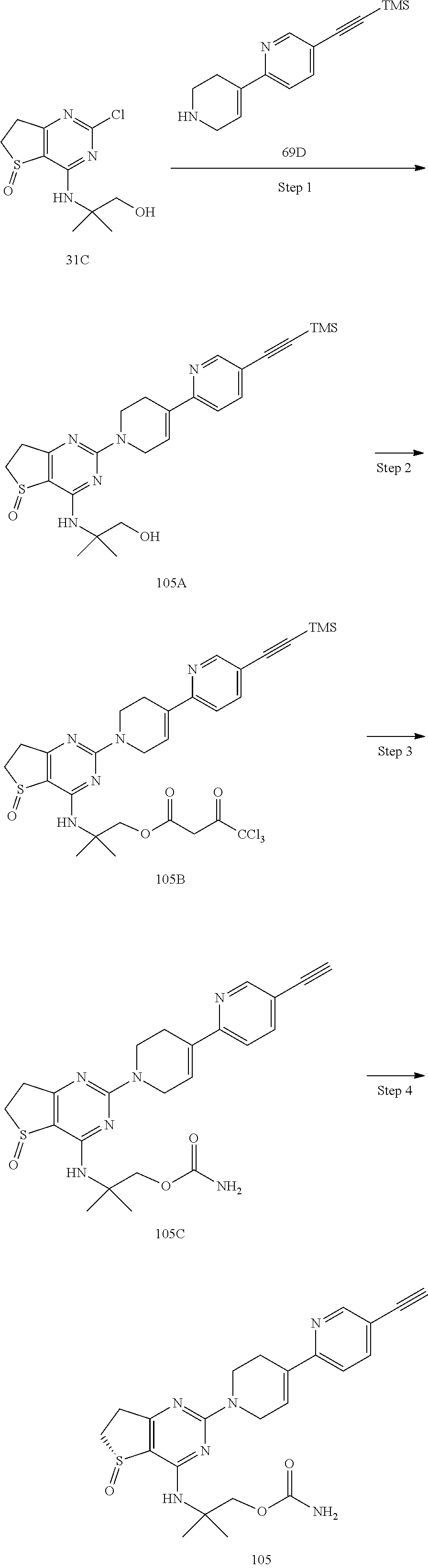

Step 1: Compound 31C (1.6 g, 5.8 mmol) was dissolved in 1,4-dioxane (30 mL), and compound 69D (1.5 g, 5.8 mmol) and N,N-diisopropylethylamine (2.2 g, 17.4 mmol) were then added and stirred at 90 degrees Celsius overnight under nitrogen protection. After the reaction was complete, the reaction liquid was concentrated and subjected to silica gel column chromatography (dichloromethane:methanol=20:1) to obtain compound 105A (2.4 g, 83%).

LC-MS (ESI): m/z=496.1 [M+H]⁺

Step 2: Compound 105A (2.4 g, 4.8 mmol) was dissolved in dichloromethane (10.0 mL), trichloroacetyl isocyanate (1.2 g, 6.3 mmol) was added in an ice bath and stirred in the ice bath for one hour, and the reaction liquid was concentrated to obtain compound 105B (2.5 g, 75%), which was directly used for the next step.

Step 3: Compound 105B (2.5 g, 3.6 mmol) was dissolved in methanol (20 mL), and potassium carbonate (2.0 g, 14.6 mmol) and water (20 mL) were added in an ice bath and stirred at room temperature 2.5 hours. The reaction liquid was diluted by adding water and extracted with dichloromethane, and the organic phases were combined, dried, filtered, and concentrated to obtain compound 105C (1.5 g, 84%).

LC-MS (ESI): m/z=467.1 [M+H]⁺

Step 4: Compound 105C was subjected to chiral SFC resolution to obtain compound 105 (1.0 g).

Preparation method: instrument: Waters 150 SFC, column: Chiralcel AD column, mobile phase: (A for CO₂ and B for 0.1% NH₃·H₂O in IPA and ACN; gradient: 55% mobile phase B, isocratic elution; flow rate: 100 mL/min, back pressure: 100 bar, column temperature: 25° C., wavelength: 220 nm, cycle time: 7.8 min, sample preparation: sample concentration 10 mg/ml, acetonitrile methanol mixed solution injection: 3.5 ml each time. retention time: 0.821 minutes. After separation, the fraction was dried in a water bath at 35° C. by a rotary evaporator to obtain a product. The solvent was dried by a lyophilizer at −80° C. to obtain the final product 105.

LC-MS (ESI): m/z=467.1 [M+H]⁺

¹H NMR (400 MHz, DMSO-d₆) δ 8.64 (d, 1H), 7.89-7.86 (m, 1H), 7.62 (d, 1H), 6.90 (s, 1H), 6.70-6.55 (m, 3H), 4.45 (s, 3H), 4.30 (s, 2H), 4.01 (t, 2H), 3.47-3.39 (m, 1H), 3.29-3.22 (m, 1H), 3.03-2.80 (m, 2H), 2.65 (s, 2H), 1.46 (d, 6H).

### Example 106

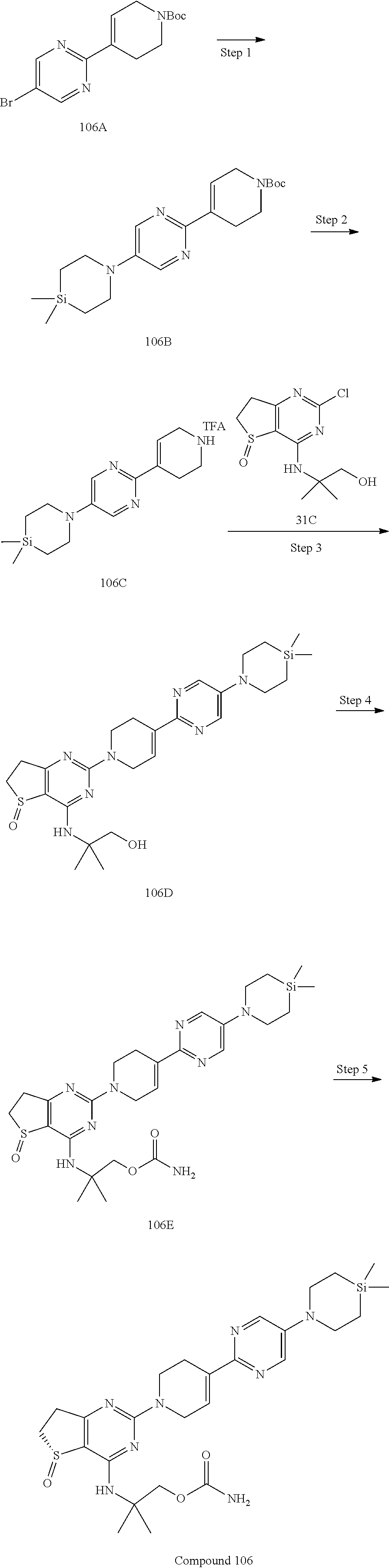

Step 1: Compound 106A (5.0 g, 14.7 mmol), 4,4-dimethyl-[1,4]silapiperidine hydrochloride (2.9 g, 17.6 mmol), bis(dibenzylideneacetone)palladium (0.7 mmol, 0.4 g), 2-(di-tert-butylphosphine)biphenyl (1.5 mmol, 0.4 g), and sodium tert-butoxide (44.1 mmol, 4.2 g) were dissolved in toluene (120 mL) and stirred at 100° C. overnight under nitrogen protection. The reaction liquid was concentrated and subjected to silica gel column chromatography (dichloromethane/methanol (v/v)=4/1) to obtain compound 106B (2.8 g, 49.1%).

¹H NMR (400 MHz, DMSO-d6) δ 8.29 (s, 2H), 6.88 (s, 1H), 3.89-3.63 (m, 6H), 2.70 (s, 2H), 1.49 (s, 9H), 0.84-0.74 (m, 4H), 0.10 (s, 6H).

Step 2: Compound 106B (2.8 g, 7.2 mmol) was dissolved in dichloromethane (20 mL), trifluoroacetic acid (10 mL) was then added, and the mixture was reacted at room temperature for half an hour and then concentrated to obtain compound 106C (2.7 g, 96.3%), which was directly used for the next step.

Using compounds 31C and 106C as raw materials, compound 106 (21 mg) was obtained according to the operation of Example 105.

Preparation method: instrument: Waters 150 Prep-SFC E, column: Chiralcel OX column, mobile phase: (phase A: CO₂, phase B: 0.1% NH₃·H₂O in MeOH and ACN; gradient: 60% mobile phase B, isocratic elution; flow rate: 100 mL/min, back pressure: 100 bar, column temperature: 25° C., wavelength: 220 nm, cycle time: 11 min, sample preparation: sample concentration 50 mg/ml, acetonitrile solution injection: 2.0 ml each time. After separation, the fraction was dried in a water bath at 35° C. by a rotary evaporator to obtain a product. The solvent was dried with a freeze-dryer at −80° C. to obtain a final product of compound 106 (retention time: 1.960 minutes).

LC-MS (ESI): m/z=571.3 [M+H]⁺

¹H NMR (400 MHz, DMSO-d₆) δ 8.39 (s, 2H), 6.94 (s, 1H), 6.66 (s, 1H), 6.66-6.36 (m, 2H), 4.42 (s, 2H), 4.30 (s, 2H), 3.98 (t, 2H), 3.77-3.62 (m, 4H), 3.47-3.39 (m, 1H), 3.27-3.22 (m, 1H), 2.99-2.85 (m, 2H), 2.65 (s, 2H), 1.46 (d, 6H), 0.77-0.62 (m, 4H), 0.08 (s, 6H).

### Example 107

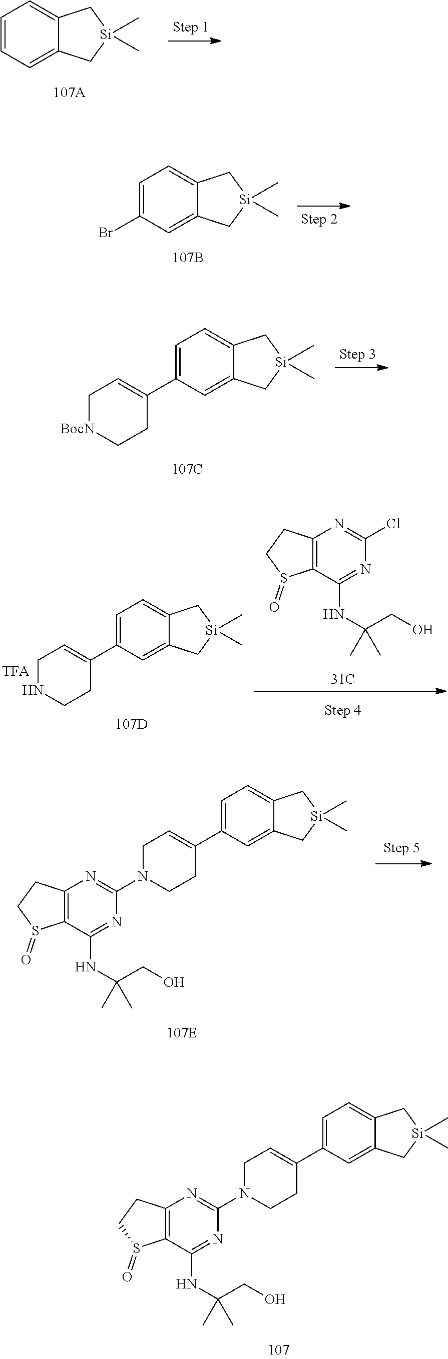

Step 1: Compound 107A (1.6 g, 10 mmol) was dissolved in hexafluoroisopropanol (20 mL), N-bromosuccinimide (2.1 g, 12 mmol) was added and reacted at room temperature for 2 h, and the system was passed through a short silica gel column, eluted with petroleum ether, and concentrated to obtain compound 107B (2.2 g, 90%).

¹H NMR (400 MHz, DMSO-d₆) δ 7.37 (s, 1H), 7.20 (d, 1H), 7.13 (d, 1H), 2.02 (s, 2H), 1.95 (s, 2H), 0.19 (s, 6H).

Step 2: Compound 107B (1.8 g, 7.6 mmol) was dissolved in 1,4-dioxane (40 mL), and 1,1-bis(diphenylphosphine)ferrocenepalladium dichloride (0.6 mg, 0.8 mmol), cesium carbonate (5.0 g, 15.4 mmol), N-Boc-1,2,5,6-tetrahydropyridine-4-boronic acid pinacol ester (2.6 g, 8.6 mmol), and water (10 mL) were then added. After nitrogen displacement three times, the mixture was heated to 90° C. and reacted for 3 h, and the system was then passed through a short silica gel column, eluted with ethyl acetate, concentrated, and subjected to column chromatography (petroleum ether/ethyl acetate=9/1) to obtain compound 107C (2.3 g, 89%).

¹H NMR (400 MHz, DMSO-d₆) δ 7.24 (s, 1H), 7.15-7.09 (m, 2H), 6.04 (s, 1H), 3.96 (s, 2H), 3.51 (t, 2H), 2.41 (s, 2H), 1.99 (s, 2H), 1.97 (s, 2H), 1.42 (s, 9H), 0.19 (s, 6H).

Step 3: Compound 107C (2.1 g, 6.0 mmol) was dissolved in dichloromethane (20 mL), trifluoroacetic acid (15 mL) was then added, and the mixture was reacted at room temperature for half an hour and then concentrated to obtain compound 107D (1.9 g, 90.4%), which was directly used for the next step.

Step 4: Compound 31C (1.0 g, 3.6 mmol) was dissolved in 1,4-dioxane (30.0 mL), compound 107D (1.5 g, 4.3 mmol) and N,N-diisopropylethylamine (1.4 g, 10.9 mmol) were then added, and the mixture was heated to 90° C. and reacted for 12 h. The system was then cooled to room temperature, concentrated, and subjected to column chromatography (dichloromethane/methanol=10/1) to obtain compound 107E (1.3 g, 64.1%).

LC-MS (ESI): m/z=483.2 [M+H]⁺

Step 5: Compound 107E (800 mg) was subjected to chiral resolution to obtain compound 107 (97 mg).

Preparation method: instrument: Waters 150 Prep-SFC E, column: Chiralcel OX column, mobile phase: (phase A: CO₂, phase B: 0.1% NH₃·H₂O in MeOH and ACN; gradient: 45% mobile phase B, isocratic elution; flow rate: 100 mL/min, back pressure: 100 bar, column temperature: 25° C., wavelength: 220 nm, cycle time: 4.8 min, sample preparation: sample concentration 10 mg/ml, acetonitrile solution injection: 2.0 ml each time. After separation, the fraction was dried in a water bath at 35° C. by a rotary evaporator to obtain a product. The solvent was dried with a freeze-dryer at −80° C. to obtain a final product of compound 107 (retention time: 0.987 minutes).

LC-MS (ESI): m/z=483.2 [M+H]⁺

¹H NMR (400 MHz, DMSO-d₆) δ 7.28 (s, 1H), 7.15 (s, 2H), 6.21 (s, 1H), 6.19 (s, 1H), 5.15 (t, 1H), 4.35 (s, 2H), 3.99 (t, 2H), 3.50 (d, 2H), 3.46-3.35 (m, 1H), 3.35-3.27 (m, 4H), 2.99-2.77 (m, 2H), 2.01 (s, 2H), 1.98 (s, 2H), 1.43 (s, 6H), 0.20 (s, 6H).

### Example 108

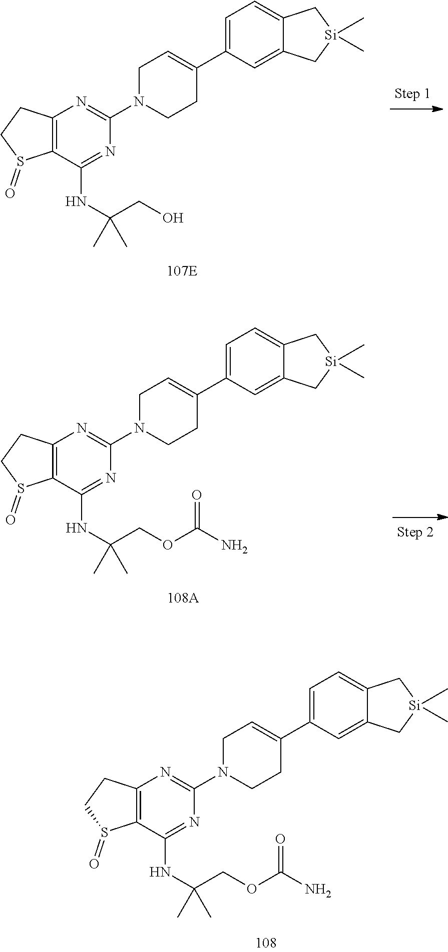 [SMILES: CC(C)(CO)Nc1nc(N2CC=C(c3ccc4c(c3)C[Si](C)(C)C4)CC2)nc2c1S(=O)CC2 ; CC(C)(COC(N)=O)Nc1nc(N2CC=C(c3ccc4c(c3)C[Si](C)(C)C4)CC2)nc2c1S(=O)CC2 ; CC(C)(COC(N)=O)Nc1nc(N2CC=C(c3ccc4c(c3)C[Si](C)(C)C4)CC2)nc2c1[S@](=O)CC2]

Using compound 107E as raw materials, compound 108 (200 mg) was obtained according to the operation of Example 105.

Preparation method: instrument: Waters 150 Prep-SFC C, column: Chiralcel AS Column, mobile phase: phase A: CO₂, phase B: 0.1% NH₃·H₂O in ETOH; gradient: 45% mobile phase B, isocratic elution; flow rate: 70 mL/min, back pressure: 100 bar, column temperature: 25° C., wavelength: 220 nm, cycle time: 7.0 min, sample preparation: sample concentration 10 mg/ml, acetonitrile solution injection: 4.0 ml each time. After separation, the fraction was dried in a water bath at 35° C. by a rotary evaporator to obtain a product. The solvent was dried with a freeze-dryer at −80° C. to obtain a final product of compound 108 (retention time: 1.779 minutes).

LC-MS (ESI): m/z=526.7 [M+H]⁺

¹H NMR (400 MHz, DMSO-d₆) δ 7.27 (s, 1H), 7.15 (s, 2H), 6.66 (s, 3H), 6.53 (s, 2H), 6.18 (s, 1H), 4.35 (s, 2H), 4.29 (s, 2H), 3.98 (t, 2H), 3.49-3.35 (m, 1H), 3.31 (s, 2H), 3.28-3.13 (m, 1H), 3.06-2.80 (m, 2H), 2.00 (s, 2H), 1.98 (s, 2H), 1.46 (d, 3H), 1.45 (d, 3H), 0.20 (s, 6H).

### Example 109

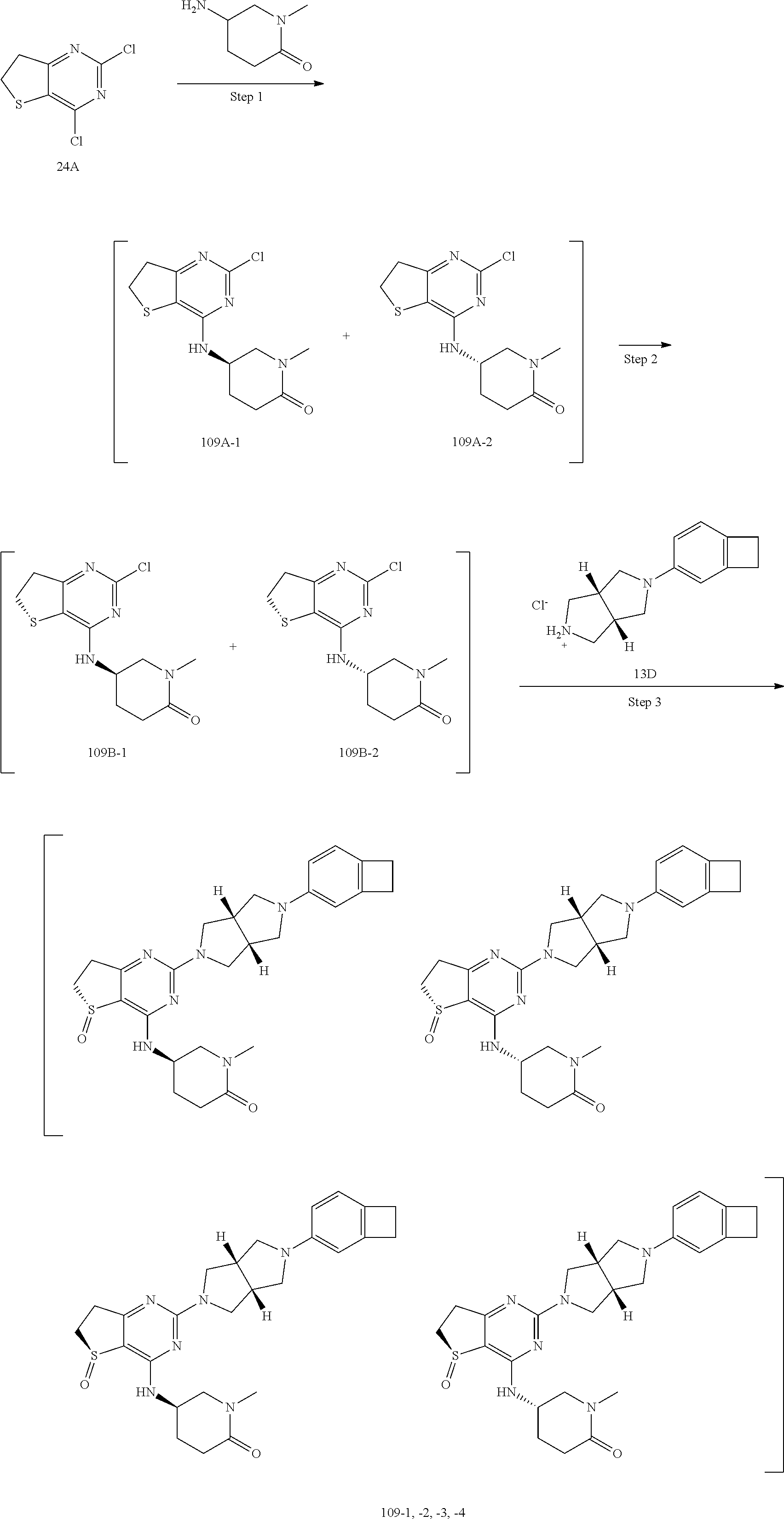

Using compound 24A and 1-methyl-5-amino-2-piperidone as raw materials, compound 109-1 (67 mg, retention time 0.98 min, 11.9%) and compound 109-2 (110 mg, retention time 1.45 min, 19.5%) were obtained according to the operation of Example 41.

Separation conditions of preparative chromatography: 1. instrument: Waters 150 Prep-SFC F; 2. chromatographic column: Chiralcel OX Column; 3. mobile phase system: A for CO₂; B for 0.1% NH₃·H₂O in MEOH and ACN; 4. gradient: B 70%; 5. flow rate: 100 mL/min.

### Compound 109-1:

¹H NMR (400 MHz, DMSO-d₆) δ 7.73 (d, 1H), 6.85 (d, 1H), 6.35 (d, 2H), 4.49 (s, 1H), 3.80 (s, 2H), 3.55-3.35 (m, 6H), 3.19 (d, 4H, 3.07 (s, 2H), 3.04-2.83 (m, 6H), 2.79 (d, 3H), 2.30 (t, 2H), 2.02-1.82 (m, 2H).

LC-MS (ESI): m/z=r493.3 [M+H]⁺

### Compound 109-2:

¹H NMR (400 MHz, DMSO-d₆) δ 7.74 (d, 1H), 6.85 (d, 1H), 6.40-6.28 (m, 2H), 4.49 (s, 1H), 3.80 (s, 2H), 3.55-3.36 (m, 6H), 3.16 (d, 4H), 3.10-2.96 (m, 6H), 2.97-2.82 (m, 2H), 2.78 (d, 3H), 2.31 (t, 2H), 1.96 (td, 2H).

LC-MS (ESI): m/z=493.3 [M+H]⁺

Using compound 109B-2 (370 mg, 1.18 mmol) as a raw material, compound 109-3 (95 mg, retention time 1.32 min, 16.4%) and compound 109-4 (110 mg, retention time 1.67 min, 21.8%) were obtained according to the above synthesis route.

### Compound 109-3:

¹H NMR (400 MHz, DMSO-d₆) δ 7.90-7.57 (m, 1H), 6.84 (d, 1H), 6.34 (d, 2H), 4.60-4.39 (m, 1H), 3.95-3.69 (m, 2H), 3.59-2.94 (m, 16H, overlapped), 2.88 (d, 2H), 2.78 (s, 3H), 2.39-2.17 (m, 2H), 1.93 (d, 2H).

LC-MS (ESI): m/z=493.3 [M+H]⁺

### Compound 109-4:

¹H NMR (400 MHz, DMSO-d₆) δ 7.73 (d, 1H), 6.85 (d, 1H), 6.35 (d, 2H), 4.49 (s, 1H), 3.80 (s, 2H), 3.56-3.35 (m, 6H), 3.19 (d, 4H), 3.07 (s, 2H), 3.01 (d, 4H), 2.97-2.84 (m, 2H), 2.79 (s, 3H), 2.30 (t, 2H), 2.03-1.82 (m, 2H).

LC-MS (ESI): m/z=493.3 [M+H]⁺

### Example 110

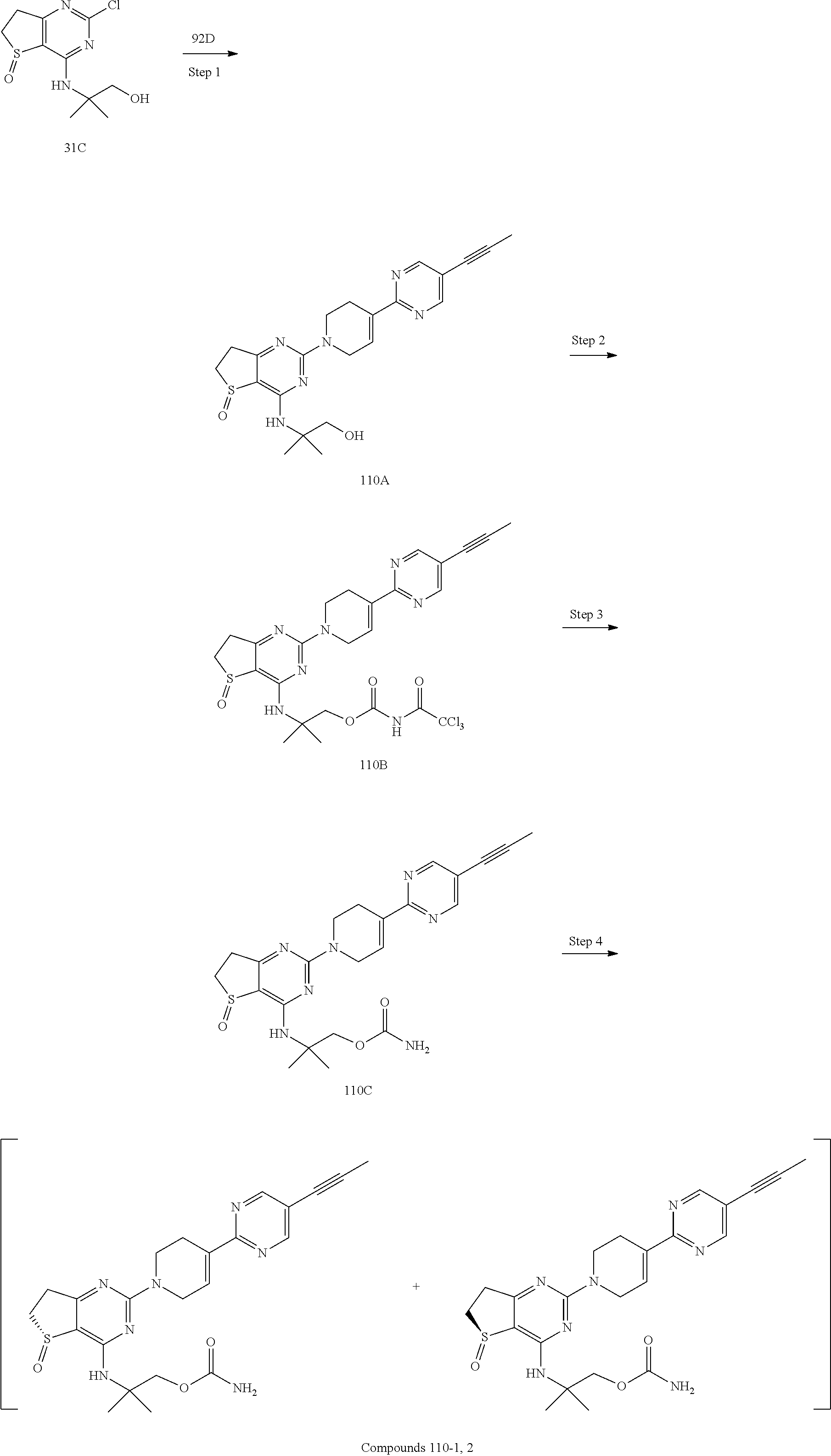

Using compounds 31C and 92D as raw materials, compound 110C (400 mg, 0.83 mmol) was obtained according to the operation of Example 84, and after chiral resolution, compound 110-1 (221.9 mg) and compound 110-2 (13.6 mg) were obtained.

Analysis method: instrument: SHIMADZU LC-30AD, column: Chiralcel IK Column; mobile phase: A: CO₂, B: 0.05% DEA in IPA and ACN; gradient: 40% B in A; flow rate: 3 mL/min, column temperature: 35° C., wavelength: 220 nm.

Preparation method: instrument: Waters 150 Prep-SFC E, column: Chiralcel IK Column (250 mm×30 mm, 10 μm) mobile phase: A: CO₂, B: 0.1% NH₃·H₂O in IPA and ACN; gradient: 50% B gradient elution, flow rate: 100 mL/min, column temperature: 25° C., wavelength: 220 nm, cycle time: 5.0 min, sample preparation: sample concentration 10 mg/ml, methanol solution injection: 2.5 ml each time. After separation, the fraction was dried by a rotary evaporator at a bath temperature of 35° C. to obtain compound 110-1 and compound 110-2.

### Compound 110-1:

¹H NMR (400 MHz, CDCl₃) δ 8.66 (s, 2H), 7.31-7.29 (m, 1H), 5.47 (s, 1H), 4.90 (br s, 2H), 4.54 (s, 2H), 4.40-4.32 (m, 2H), 4.12-4.05 (m, 2H), 3.65-3.57 (m, 1H), 3.42-3.36 (m, 1H), 3.05-2.99 (m, 2H), 2.81-2.77 (m, 2H), 2.11 (s, 3H), 1.54-1.52 (m, 6H).

LC-MS (ESI): m/z=482.2 [M+H]⁺

### Compound 110-2:

¹H NMR (400 MHz, CDCl₃) δ 8.66 (s, 2H), 7.31-7.29 (m, 1H), 5.47 (s, 1H), 4.90 (brs, 2H), 4.54 (s, 2H), 4.40-4.32 (m, 2H), 4.12-4.05 (m, 2H), 3.65-3.57 (m, 1H), 3.42-3.36 (m, 1H), 3.05-2.99 (m, 2H), 2.82-2.76 (m, 2H), 2.11 (s, 3H), 1.54-1.52 (m, 6H).

LC-MS (ESI): m/z=482.2 [M+H]⁺

### Example 111

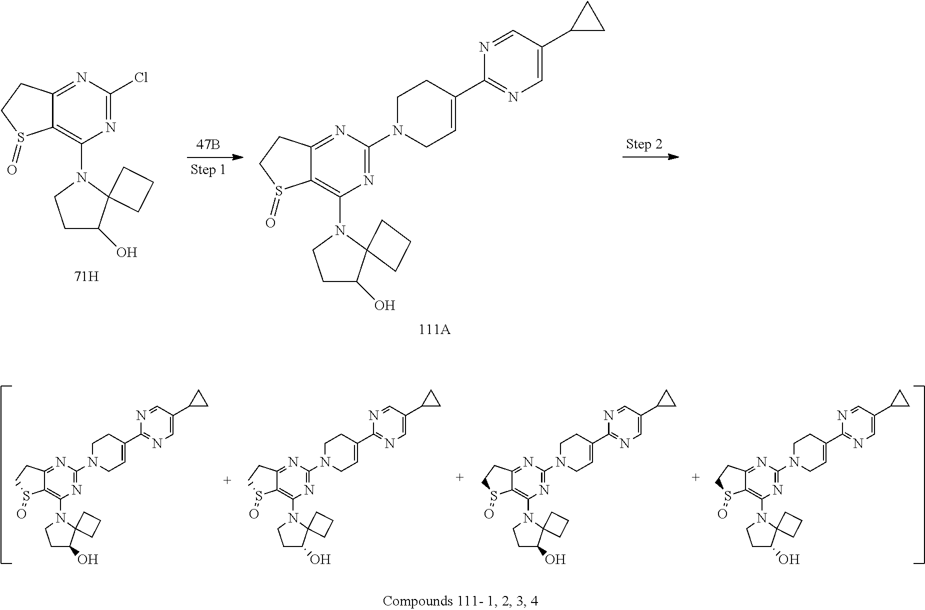

Using compounds 47B and 71H as raw materials, compound 111-1 (180.8 mg), compound 111-2 (92.7 mg), compound 111-3 (11.1 mg), and compound 111-4 (61.6 mg) were obtained according to the operation of Example 71 (steps 8 and 9).

HPLC analysis method: instrument: SHIMADZU LC-2020AD, column: C18 Column; mobile phase: A: 0.1% TFA in H₂O, B: ACN; gradient: 10-80% B in A; flow rate: 1.2 mL/min, column temperature: 45° C., wavelength: 210 nm & 254 nm.

HPLC preparation method: instrument: SHIMADZU LC-20AP, column: C18 Column; mobile phase: A: 0.225% FA in H₂O, B: ACN; gradient: 55-85% B gradient elution; flow rate: 75 mL/min, column temperature: 25° C.; wavelength: 220 nm; cycle time: 5.0 min, sample preparation: sample concentration 50 mg/ml, methanol solution injection: 5.0 ml each time. After separation, the fraction was dried by a rotary evaporator at a bath temperature of 35° C. to obtain compound P1 and compound P2.

SFC analysis method: instrument: SHIMADZU LC-30AD, column: Chiralcel IC Column; mobile phase: A:CO₂, B: 0.05% DEA in EtOH and ACN; gradient: 60% B in A; flow rate: 3 mL/min, column temperature: 35° C., wavelength: 220 nm.

SFC preparation method: instrument: Waters 150 Prep-SFC F, column: Chiralcel Cellulose-2 Column; mobile phase: A: CO₂, B: 0.1% NH3·H2O in IPA and ACN; gradient: 60% B gradient elution; flow rate: 100 mL/min, column temperature: 25° C.; wavelength: 220 nm; cycle time: 8.6 min; sample preparation: sample concentration 10 mg/ml, acetonitrile solution injection: 1.5 ml each time. After separation, the fraction was dried by a rotary evaporator at a bath temperature of 35° C. to obtain compound 111-1 (180.8 mg), compound 111-2 (92.7 mg), compound 111-3 (11.1 mg), and compound 111-4 (61.6 mg).

Compound 111-1, retention time: 1.059 min; ¹H NMR (400 MHz, CDCl₃) δ 8.43 (s, 2H), 7.24-7.22 (m, 1H), 4.67-4.53 (m, 2H), 4.27 (s, 1H), 4.22-4.06 (m, 4H), 3.83-3.71 (m, 2H), 3.51-3.43 (m, 1H), 3.17-3.12 (m, 1H), 3.07-3.01 (m, 2H), 2.82 (s, 2H), 2.25-2.18 (m, 2H), 2.06-1.98 (m, 2H), 1.89-1.76 (m, 2H), 1.66-1.61 (m, 2H), 1.11-1.06 (m, 2H), 0.79-0.75 (m, 2H).

LC-MS (ESI): m/z=479.3 [M+H]⁺

Compound 111-2, retention time: 1.245 min; ¹H NMR (400 MHz, CDCl₃) δ 8.43 (s, 2H), 7.24-7.22 (m, 1H), 4.67-4.53 (m, 2H), 4.27 (s, 1H), 4.22-4.06 (m, 4H), 3.83-3.71 (m, 2H), 3.51-3.43 (m, 1H), 3.17-3.12 (m, 1H), 3.07-3.01 (m, 2H), 2.82 (s, 2H), 2.46-2.18 (m, 2H), 2.08-1.98 (m, 2H), 1.89-1.76 (m, 2H), 1.66-1.61 (m, 2H), 1.11-1.06 (m, 2H), 0.79-0.75 (m, 2H).

LC-MS (ESI): m/z=479.3 [M+H]⁺

Compound 111-3, retention time: 1.493 min; ¹H NMR (400 MHz, CDCl₃) δ 8.43 (s, 2H), 7.24-7.22 (m, 1H), 4.61 (s, 2H), 4.23-4.22 (s, 1H), 4.19-4.12 (m, 3H), 3.78-3.63 (m, 3H), 3.49-3.41 (m, 1H), 3.16-3.10 (m, 3H), 2.83 (s, 2H), 2.26-2.21 (m, 1H), 2.09-2.06 (m, 2H), 2.00-1.92 (m, 1H), 1.89-1.83 (m, 2H), 1.72-1.67 (m, 2H), 1.11-1.06 (m, 2H), 0.80-0.75 (m, 2H).

LC-MS (ESI): m/z=479.3 [M+H]⁺

Compound 111-4, retention time: 2.368 min; ¹H NMR (400 MHz, CDCl₃) δ 8.43 (s, 2H), 7.24-7.22 (m, 1H), 4.62-4.60 (s, 2H), 4.23-4.22 (s, 1H), 4.19-4.12 (m, 3H), 3.78-3.63 (m, 3H), 3.49-3.41 (m, 1H), 3.16-3.10 (m, 3H), 2.83 (s, 2H), 2.26-2.21 (m, 1H), 2.09-2.06 (m, 2H), 2.00-1.92 (m, 1H), 1.89-1.83 (m, 2H), 1.72-1.67 (m, 2H), 1.11-1.06 (m, 2H), 0.80-0.75 (m, 2H).

LC-MS (ESI): m/z=479.3 [M+H]⁺

### Example 112

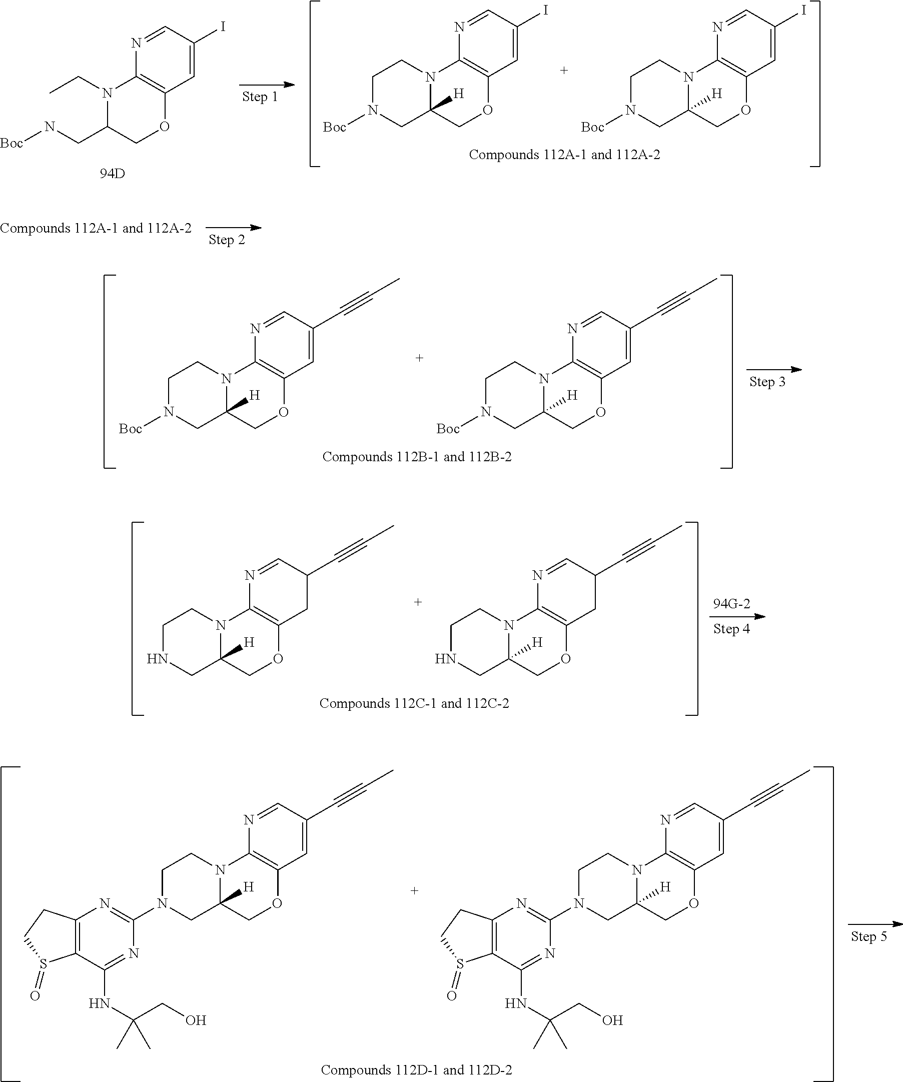

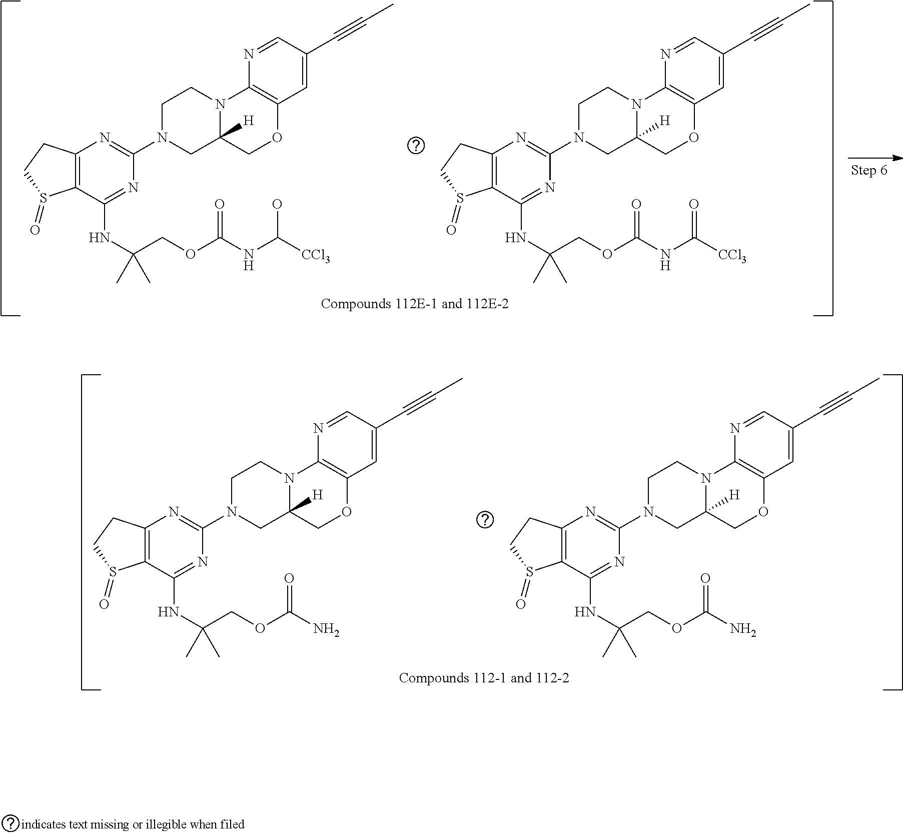

Step 1: Compound 94D (1.5 g) was separated by SFC to obtain two isomers, i.e., compound 112A-1 (700 mg, retention time 5.86 min, 47%) and compound 112A-2 (700 mg, retention time 7.27 min, 47%).

Separation conditions of preparative chromatography: 1. instrument: Waters 150 AP-SFC; 2. chromatographic column: AD; 3. mobile phase system: A for CO₂; B for MEOH; 4. gradient: B 28%; and 5. flow rate: 40 mL/min

LC-MS (ESI): m/z=418.0 [M+H]⁺

Step 2: At room temperature in a nitrogen atmosphere, compound 112A-1 (700 mg, 1.68 mmol), propyne (1 M/L, 33.6 mL, 33.6 mmol), bis(triphenylphosphine)palladium dichloride (119.3 mg, 0.17 mmol), cuprous iodide (64.6 mg, 0.34 mmol), and tetrahydrofuran (10 mL) were added to a 50 mL round-bottom flask, followed by triethylamine (509.1 mg, 5.04 mmol), and the mixture was reacted at this temperature for 12 h. After the reaction was complete, water (5 mL) was added, and the mixture was extracted twice with ethyl acetate (10 mL). The organic phases were combined, washed with a saturated sodium chloride aqueous solution, and dried over anhydrous sodium sulfate. After filtration, the reaction liquid was concentrated under reduced pressure and then separated and purified by silica gel column chromatography (eluents: EA/PE (v/v)=0-30%) to obtain the target compound 112B-1 (500 mg, 90%).

LC-MS (ESI): m/z=330.2 [M+H]⁺

According to the above operation, using compound 112A-2 (700 mg, 1.68 mmol) as a raw material, the target compound 112B-2 (500 mg, 90%) was obtained.

LC-MS (ESI): m/z=330.2 [M+H]⁺

Step 3: At room temperature, 112B-1 (500 mg, 1.52 mmol), dichloromethane (4 mL), and trifluoroacetic acid (1 mL) were added to a 25 mL round-bottom flask, and the mixture was continuously stirred at this temperature for 2 h. After the reaction was complete, the reaction liquid was concentrated under reduced pressure to obtain the target compound 112C-1 (325 mg, 93%).

LC-MS (ESI): m/z=230.2 [M+H]⁺

According to the above operation, using compound 112B-2 (500 mg, 1.52 mmol) as a raw material, the target compound 112C-2 (330 mg, 95%) was obtained.

LC-MS (ESI): m/z=230.2 [M+H]⁺

Step 4: At room temperature, compound 112C-1 (325 mg, 1.42 mmol), compound 94G-2 (429.9 mg, 1.56 mmol), and 1,4-dioxane (5 mL) were added to a 25 mL round-bottom flask, and N,N-diisopropylethylamine (545.3 mg, 4.26 mmol) were added at room temperature, heated to 100° C. and reacted for 12 h. After the reaction was complete, the reaction liquid was concentrated under reduced pressure and then separated and purified by silica gel column chromatography (eluents: MeOH/DCM (v/v)=0-10%) to obtain the target compound 112D-1 (600 mg, 90%).

LC-MS (ESI): m/z=469.2 [M+H]⁺

According to the above operation, using compound 112C-2 (330 mg, 1.44 mmol) as a raw material, the target compound 112D-2 (620 mg, 92%) was obtained.

LC-MS (ESI): m/z=469.2 [M+H]⁺

Step 5: At room temperature, compound 112D-1 (300 mg, 0.64 mmol) and dichloromethane (5 mL) were added to a 25 mL round-bottom flask, and trichloroacetyl isocyanate (144.7 mg, 0.77 mmol) was added in an ice bath and stirred in the ice bath for 1 h. After the reaction was complete, the reaction liquid was concentrated under reduced pressure and then separated and purified by silica gel column chromatography (eluents: MeOH/DCM (v/v)=0-10%) to obtain the target compound 112E-1 (300 mg, 71%).

LC-MS (ESI): m/z=656.2 [M+H]⁺

According to the above operation, using compound 112D-2 (300 mg, 0.64 mmol) as a raw material, the target compound 112E-2 (300 mg, 71%) was obtained.

LC-MS (ESI): m/z=656.2 [M+H]⁺

Step 6: At room temperature, compound 112E-1 (150 mg, 0.23 mmol) and methanol (2 mL) were added to a 25 mL round-bottom flask, and potassium carbonate (95.2 mg, 0.69 mmol) and water (2 mL) were added in an ice bath and reacted at room temperature for 2.5 h. After the reaction was complete, the reaction liquid was concentrated under reduced pressure and then separated and purified by silica gel column chromatography (eluents: MeOH/EA (v/v)=0-10%) to obtain the target compound 112-1 (100 mg, 86%)

According to the above operation, using compound 112E-2 (150 mg, 0.23 mmol) as a raw material, the target compound 112-2 (100 mg, 86%) was obtained.

### Compound 112-1:

¹H NMR (400 MHz, Chloroform-d) δ 7.85 (d, 1H), 6.95 (d, 1H), 5.53 (brs, 1H), 5.00-4.74 (m, 4H), 4.62-4.56 (m, 1H), 4.39-4.30 (m, 3H), 4.03-3.98 (m, 1H), 3.62-3.53 (m, 1H), 3.44-3.35 (m, 2H), 3.17-3.08 (m, 1H), 3.05-2.94 (m, 2H), 2.89-2.75 (m, 2H), 2.03 (s, 3H), 1.51 (s, 3H), 1.48 (s, 3H).

LC-MS (ESI): m/z=512.2 [M+H]⁺

### Compound 112-2:

¹H NMR (400 MHz, DMSO-d₆) δ 7.78 (d, 1H), 6.98 (d, 1H), 6.74 (brs, 1H), 6.68-6.44 (m, 2H), 4.82-4.71 (m, 2H), 4.51-4.39 (m, 2H), 4.36-4.25 (m, 1H), 4.04-3.94 (m, 1H), 3.49-3.37 (m, 2H), 3.29-3.20 (m, 2H), 3.12-3.02 (m, 1H), 3.01-2.92 (m, 1H), 2.92-2.84 (m, 1H), 2.84-2.73 (m, 2H), 2.00 (s, 3H), 1.44 (s, 3H), 1.42 (s, 3H).

LC-MS (ESI): m/z=512.2 [M+H]⁺

### Example 113

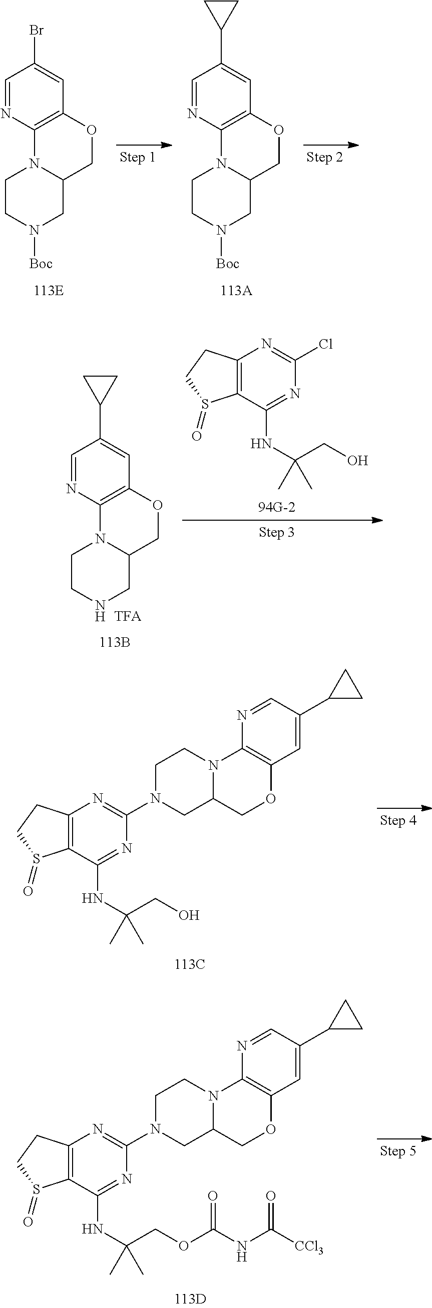

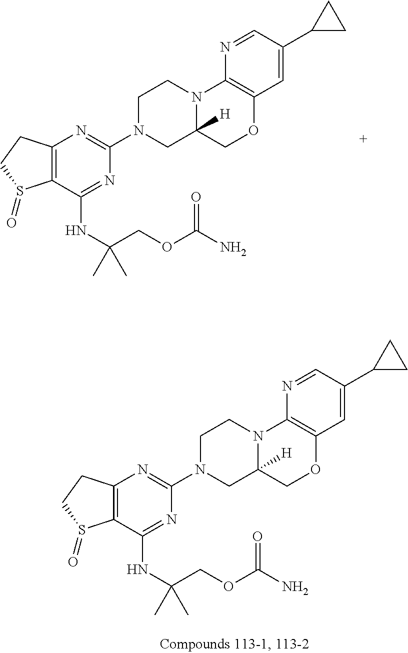

Step 1: To a 100 mL single-mouth flask, 113E (1.2 g, 3.25 mmol, see WO 2022184103 for the synthesis), dioxane (15 mL), water (3 mL), 1,1-bis(diphenylphosphine)ferrocenepalladium dichloride (240 mg, 0.33 mmol), potassium carbonate (1.35 g, 9.75 mmol), and cyclopropylboronic acid (280 mg, 3.25 mmol) were successively added and reacted at 80° C. overnight under nitrogen protection. After concentration under reduced pressure, the system was then separated and purified by silica gel column chromatography (eluents: ethyl acetate/petroleum ether (v/v)=20/100) to obtain 113A (800 mg, yield 74.4%).

LC-MS (ESI): m/z=332.2 [M+H]⁺.

Using 113A as a raw material, 150 mg of a mixture of compounds 113-1 and 113-2 was synthesized according to the operation of Example 112.

Chiral preparation was carried out to obtain the title compound 113-1 (53 mg, two-step yield 16.2%, retention time: 0.883 min) and the title compound 113-2 (65 mg, 19.8%, retention time: 1.147 min). Chiral preparation method: instrument: Waters 150 MGM; chromatographic column: Chiralpak Column; mobile phase: A: carbon dioxide, and B: ethanol (0.1% aqueous ammonia); isocratic elution: 70% mobile phase B; flow rate: 100 mL/min; back pressure: 100 bar; column temperature: 25° C.; wavelength: 220 nm; elution time: 4.0 min.

### (Compound 113-1)

¹H NMR (400 MHz, DMSO-d₆) δ 7.63-7.58 (m, 1H), 6.71 (s, 1H), 6.67-6.66 (m, 1H), 6.56 (s, 2H), 4.85-4.66 (m, 2H), 4.46-4.21 (m, 4H), 4.04-3.90 (m, 1H), 3.52-3.35 (m, 1H), 3.29-3.22 (m, 2H), 3.09-2.63 (m, 5H), 1.85-1.72 (m, 1H), 1.47-1.39 (m, 6H), 0.89-0.81 (m, 2H), 0.60-0.55 (m, 2H).

LC-MS (ESI): m/z=514.2 [M+H]⁺.

### (Compound 113-2)

¹H NMR (400 MHz, DMSO-d₆) δ 7.62-7.59 (m, 1H), 6.70 (s, 1H), 6.67-6.64 (m, 1H), 6.56 (s, 2H), 4.89-4.55 (m, 2H), 4.48-4.19 (m, 4H), 4.03-3.83 (m, 1H), 3.53-3.38 (m, 1H), 3.29-3.22 (m, 2H), 3.06-2.65 (m, 5H), 1.85-1.72 (m, 1H), 1.46-1.39 (m, 6H), 0.87-0.82 (m, 2H), 0.60-0.55 (m, 2H).

LC-MS (ESI): m/z=514.2 [M+H]⁺.

### Example 114

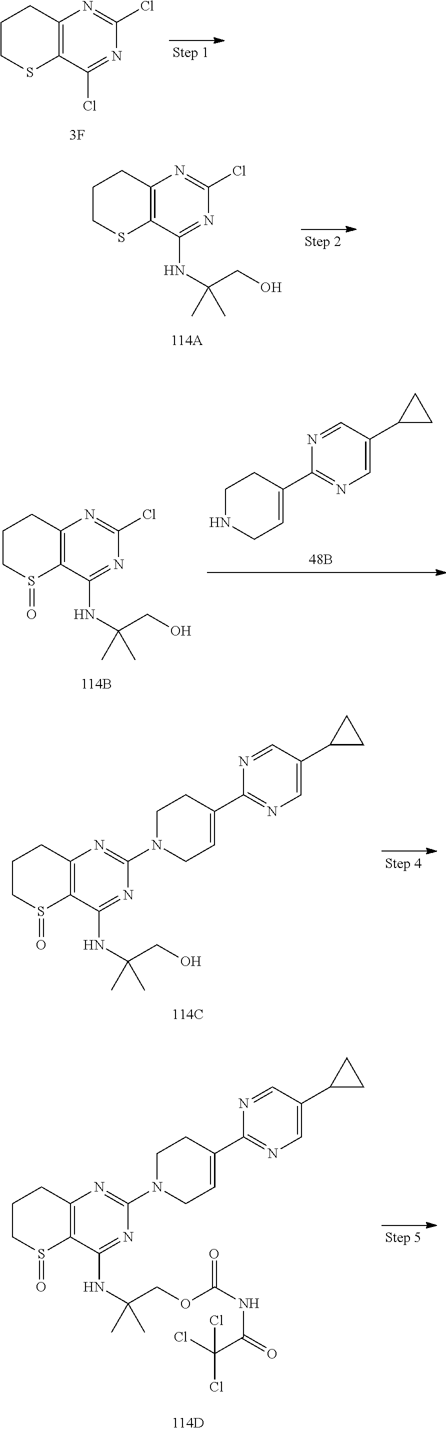 [SMILES: Clc1nc(Cl)c2c(n1)CCCS2 ; CC(C)(CO)Nc1nc(Cl)nc2c1SCCC2 ; CC(C)(CO)Nc1nc(Cl)nc2c1S(=O)CCC2 ; C1=C(c2ncc(C3CC3)cn2)CCNC1 ; CC(C)(CO)Nc1nc(N2CC=C(c3ncc(C4CC4)cn3)CC2)nc2c1S(=O)CCC2 ; CC(C)(COC(=O)NC(=O)C(Cl)(Cl)Cl)Nc1nc(N2CC=C(c3ncc(C4CC4)cn3)CC2)nc2c1S(=O)CCC2]

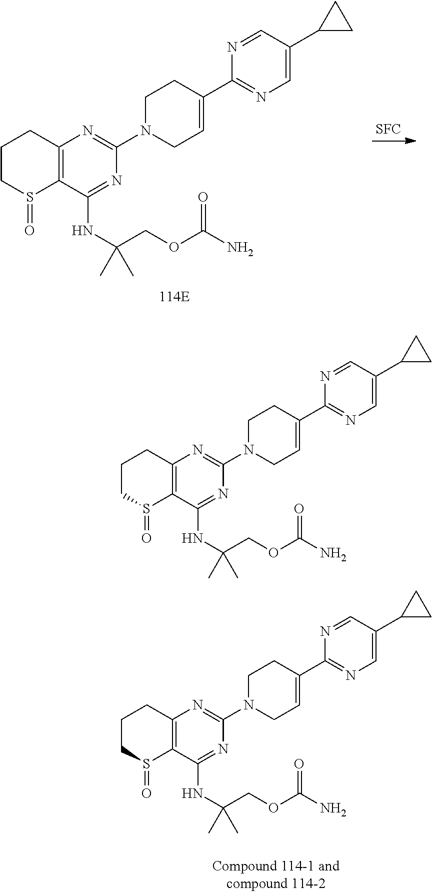 [SMILES: CC(C)(COC(N)=O)Nc1nc(N2CC=C(c3ncc(C4CC4)cn3)CC2)nc2c1S(=O)CCC2 ; "SFC" ; CC(C)(COC(N)=O)Nc1nc(N2CC=C(c3ncc(C4CC4)cn3)CC2)nc2c1[S@](=O)CCC2 ; CC(C)(COC(N)=O)Nc1nc(N2CC=C(c3ncc(C4CC4)cn3)CC2)nc2c1[S@@](=O)CCC2]

Using 3F and 2-amino-2-methylpropan-1-ol as raw materials, compound 114-1 (50 mg) and compound 114-2 (45 mg) were synthesized according to the operation of Example 88.

Chiral resolution method: instrument: Waters 150 SFC; column: Chiralpak IC Column (250×30 mm, I.D 30 mm, 10 μm particle size); mobile phase: A for CO₂ and B for IPA+ACN (0.1% NH₃·H₂O); gradient: 45% phase B isocratic elution; flow rate: 100 mL/min; back pressure: 100 bar; column temperature: 25° C.; wavelength: 220 nm.

LCMS m/z=498.2 [M+1]⁺

Compound 114-1, H NMR (400 MHZ, DMSO-d₆) δ 8.54 (s, 2H), 7.18 (s, 1H), 6.52 (s, 2H), 6.18 (s, 1H), 4.42 (s, 2H), 4.27 (s, 2H), 3.95 (t, 2H), 2.95-3.10 (m, 2H), 2.56-2.73 (m, 4H), 2.25-2.35 (m, 1H), 1.91-1.98 (m, 2H), 1.46 (s, 6H), 1.00-1.06 (m, 2H), 0.82-0.86 (m, 2H).

Compound 114-2, ˜H NMR (400 MHZ, DMSO-d) n 8.54 (s, 2H), 7.18 (s, 1H), 6.52 (s, 2H), 6.18 (s, 1H), 4.42 (s, 2H), 4.27 (s, 2H), 3.95 (t, 2H), 2.95-3.10 (m, 2H), 2.56-2.73 (m, 4H), 2.25-2.35 (m, 1H), 1.91-1.98 (m, 2H), 1.46 (s, 6H), 1.00-1.06 (m, 2H), 0.82-0.86 (m, 2H).

### Example 115

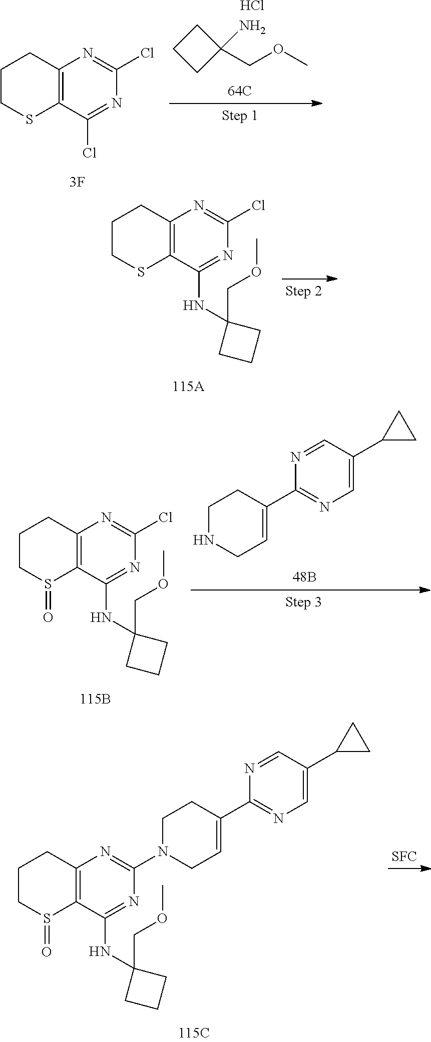

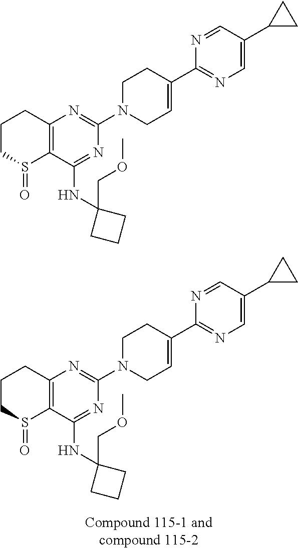 [SMILES: COCC1(Nc2nc(N3CC=C(c4ncc(C5CC5)cn4)CC3)nc3c2[S@](=O)CCC3)CCC1 ; COCC1(Nc2nc(N3CC=C(c4ncc(C5CC5)cn4)CC3)nc3c2[S@@](=O)CCC3)CCC1]

Using 3F and 64C as raw materials, compound 115-1 (110 mg) and compound 115-2 (100 mg) were synthesized according to the operation of Example 64.

Chiral resolution method: instrument: Waters 150 SFC; column: Chiralpak IC Column (250×30 mm, I.D 30 mm, 10 μm particle size); mobile phase: A for CO₂ and B for IPA+ACN (0.1% NH₃·H₂O); gradient: 60% phase B isocratic elution; flow rate: 100 mL/min; back pressure: 100 bar; column temperature: 25° C.; wavelength: 220 nm.

LCMS m/z=481.2 [M+1]⁺

Compound 115-1, ¹H NMR (400 MHZ, DMSO-d₆) δ 8.53 (s, 2H), 7.18 (s, 1H), 6.60 (s, 1H), 4.39 (s, 2H), 3.93 (t, 2H), 3.69-3.75 (m, 2H), 3.30 (s, 3H), 3.04-3.08 (m, 1H), 2.90-2.96 (m, 1H), 2.56-2.72 (m, 4H), 2.30-2.42 (m, 3H), 2.14-2.25 (m, 2H), 1.81-1.98 (m, 4H), 1.00-1.05 (m, 2H), 0.82-0.85 (m, 2H).

Compound 115-2, ¹H NMR (400 MHZ, DMSO-d₆) δ 8.53 (s, 2H), 7.18 (s, 1H), 6.60 (s, 1H), 4.39 (s, 2H), 3.93 (t, 2H), 3.69-3.75 (m, 2H), 3.30 (s, 3H), 3.04-3.08 (m, 1H), 2.90-2.96 (m, 1H), 2.56-2.72 (m, 4H), 2.30-2.42 (m, 3H), 2.14-2.25 (m, 2H), 1.81-1.98 (m, 4H), 1.00-1.05 (m, 2H), 0.82-0.85 (m, 2H).

### Example 116

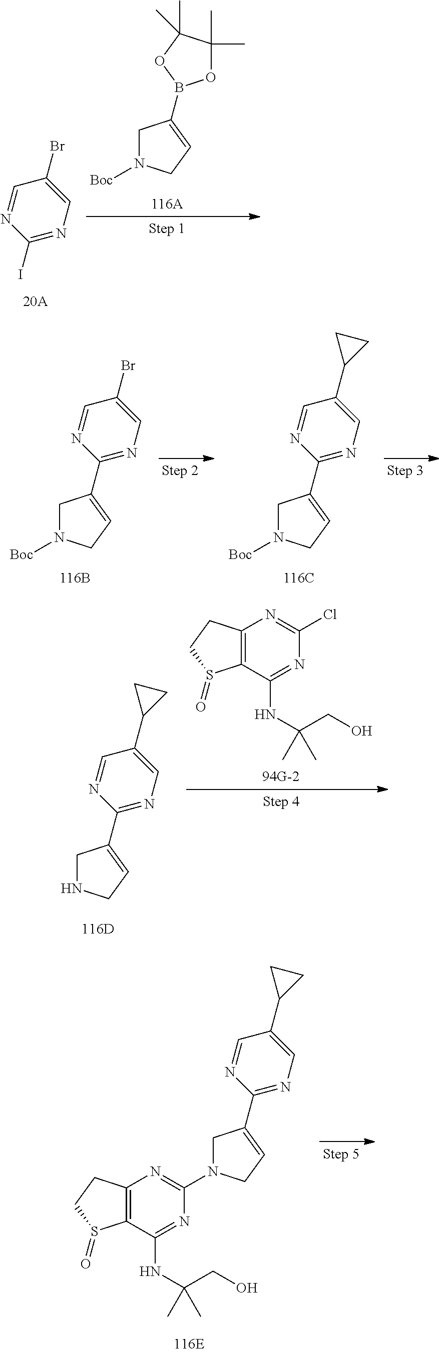

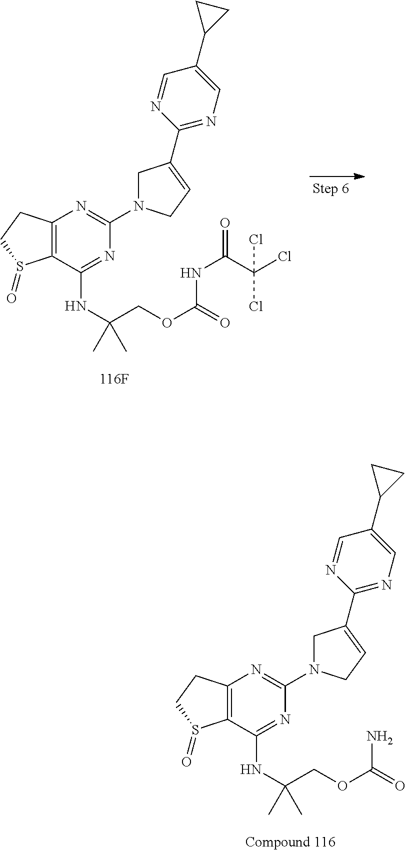

Step 1: Compound 20A (5 g, 17.55 mmol) and compound 116A (5.18 g, 17.55 mmol) were dissolved in tetrahydrofuran (40 mL), and 1,1-bis(diphenylphosphine)ferrocenepalladium dichloride (1.28 g, 1.76 mmol), potassium carbonate (7.28 g, 52.57 mmol), and water (10 mL) were then added. After nitrogen displacement three times, the system was heated to 80° C. and reacted for 14 h. After the reaction was complete as detected by a TLC spotting plate (petroleum ether:ethyl acetate=5:1), water (50 mL) was added and stirred for 5 min, and the mixture was extracted with ethyl acetate (30 mL). The organic phase was separated, dried over anhydrous sodium sulfate, and concentrated under reduced pressure, and the crude product was purified by column chromatography (eluents: petroleum ether:ethyl acetate=5:1) to obtain the target compound 116B (3.5 g, 61.14%).

LC-MS (ESI): m/z=326.1 [M+1]⁺.

Using 116B as a raw material, compound 116 (200 mg, 66%) was synthesized according to the operation of Example 47.

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A with a content of 40-80%; c. flow: 15 mL/min; d. elution time: 20 min; retention time: 12.00 min.

LCMS m/z=470.2 [M+1]⁺

¹H NMR (400 MHZ, DMSO-d₆) δ 8.62 (s, 1H), 8.58 (s, 1H), 6.94 (s, 1H), 6.47-6.69 (m, 3H), 4.68 (d, 2H), 4.53 (d, 2H), 4.30 (d, 2H), 3.41-3.50 (m, 1H), 3.23-3.28 (m, 1H), 2.86-3.02 (m, 2H), 1.92-2.00 (m, 1H), 1.46-1.51 (m, 6H), 1.01-1.08 (m, 2H), 0.82-0.89 (m, 2H).

### Example 117

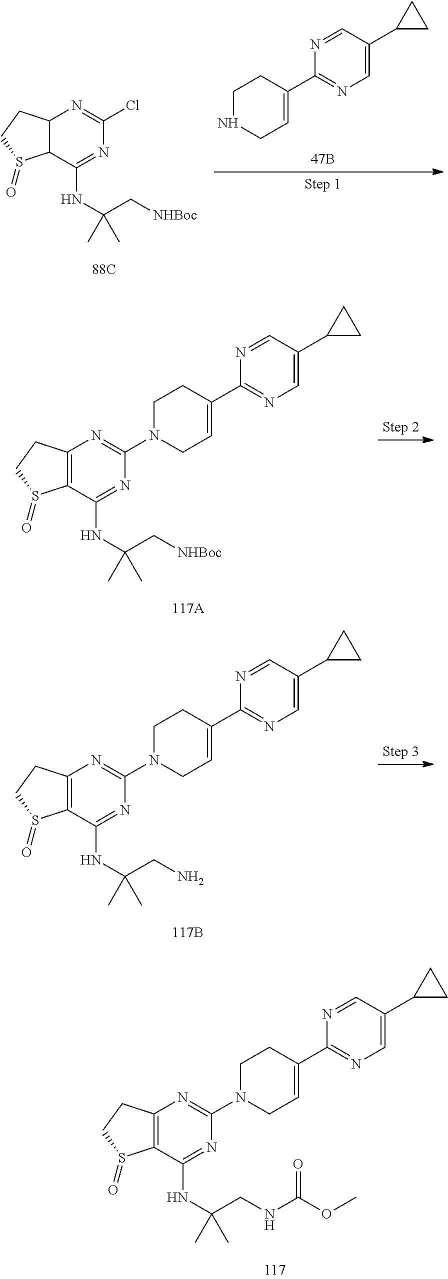

Using 47B and 88C as raw materials, compound 117 (1.0 g, 50%) was synthesized according to the operation of Example 88.

Preparation method: instrument: Waters 150 Prep-SFC C, column: Chiralcel AS Column, mobile phase: (A for CO₂; B for MEOH); gradient: 45% mobile phase B, isocratic elution; flow rate: 70 mL/min, back pressure: 100 bar, column temperature: 25° C., wavelength: 220 nm, cycle time: 6.0 min, sample preparation: sample concentration 10 mg/ml, acetonitrile solution injection: 3 ml each time. After separation, the fraction was dried on a rotary evaporator at a bath temperature of 35° C. to obtain 117 (retention time: 1.977 minutes).

LC-MS (ESI): m/z=498.1 [M+1]⁺

¹H NMR (400 MHz, DMSO-d6) δ 8.54 (s, 2H), 7.45 (t, 1H), 7.19 (s, 1H), 6.69 (s, 1H), 4.47 (s, 2H), 4.00 (t, 2H), 3.57 (s, 3H), 3.48-3.37 (m, 2H), 3.28-3.19 (m, 2H), 2.99-2.83 (m, 2H), 2.67 (s, 2H), 1.93 (m, 1H), 1.43 (d, 6H), 1.12-0.99 (m, 2H), 0.91-0.76 (m, 2H).

### Example 118

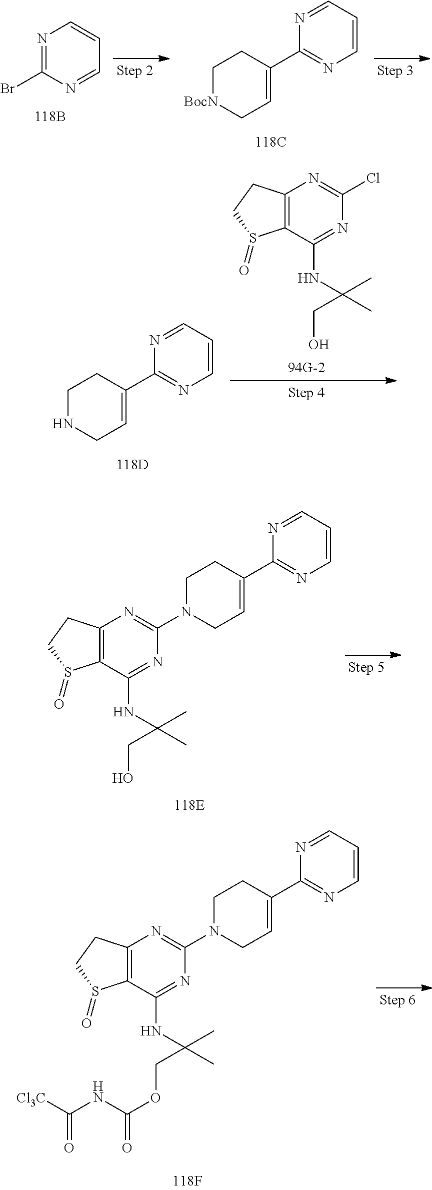

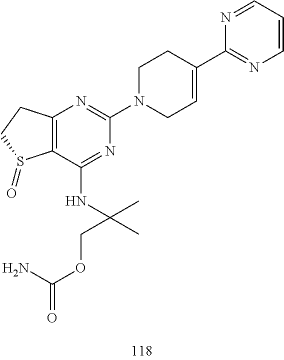 [SMILES: CC(C)(COC(N)=O)Nc1nc(N2CC=C(c3ncccn3)CC2)nc2c1[S@](=O)CC2]

Using 118B and N-Boc-1,2,5,6-tetrahydropyridine-4-boronic acid pinacol ester as raw materials, compound 118 (800 mg, 61%) was synthesized according to the operation of Example 47.

LC-MS (ESI): m/z=444.2 [M+H]⁺

¹H NMR (400 MHz, DMSO-d₆) δ 8.79 (d, 2H), 7.35 (t, 1H), 7.31-7.25 (m, 1H), 6.75-6.40 (m, 3H), 4.49 (s, 2H), 4.30 (s, 2H), 4.01 (t, 2H), 3.49-3.38 (m, 1H), 3.29-3.21 (m, 1H), 3.02-2.92 (m, 1H), 2.91-2.83 (m, 1H), 2.70 (s, 2H), 1.47 (d, 6H).

### Example 119

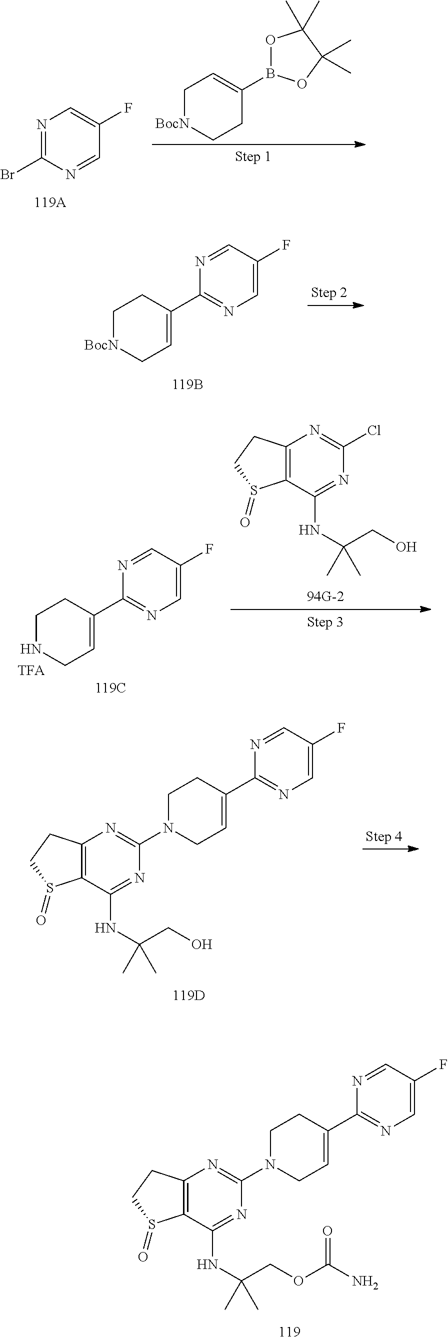

Using 119A and N-Boc-1,2,5,6-tetrahydropyridine-4-boronic acid pinacol ester as raw materials, compound 119 (470 mg, 51%) was synthesized according to the operation of Example 47.

LC-MS (ESI): m/z=462.2 [M+H]⁺

¹H NMR (400 MHz, DMSO-d₆) δ 8.86 (s, 2H), 7.22 (s, 1H), 6.64 (s, 1H), 6.56 (s, 2H), 4.48 (s, 2H), 4.30 (s, 2H), 4.01 (t, 2H), 3.55-3.37 (m, 1H), 3.28-3.22 (m, 1H), 3.03-2.92 (m, 1H), 2.92-2.86 (m, 1H), 2.69 (s, 2H), 1.46 (d, 6H).

¹⁹F NMR (376 MHz, DMSO-d₆) δ −138.83 (s, 1F).

### Example 120

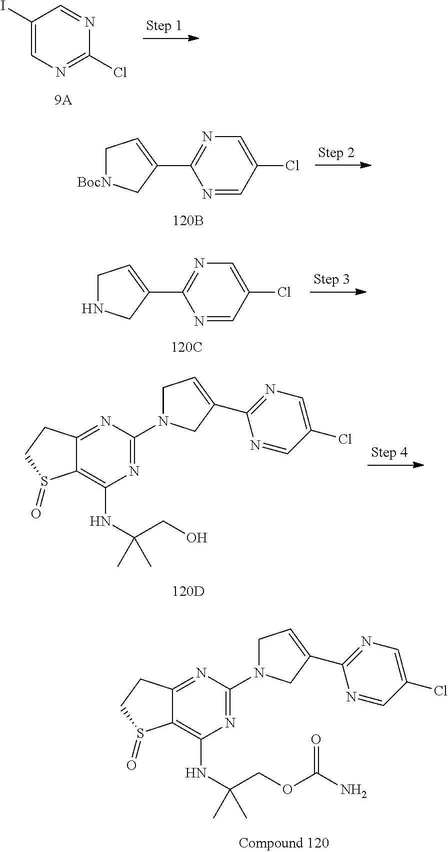 [SMILES: Clc1ncc(I)cn1 ; CC(C)(C)OC(=O)N1CC=C(c2ncc(Cl)cn2)C1 ; Clc1cnc(C2=CCNC2)nc1 ; CC(C)(CO)Nc1nc(N2CC=C(c3ncc(Cl)cn3)C2)nc2c1[S@](=O)CC2 ; CC(C)(COC(N)=O)Nc1nc(N2CC=C(c3ncc(Cl)cn3)C2)nc2c1[S@](=O)CC2]

Using compound 9A as a raw material, the target compound 120 (10 mg, 4%) was obtained according to the synthesis step of Example 116.

LCMS (ESI): m/z=464.2 [M+H]⁺

¹H NMR (400 MHz, DMSO-d₆) δ 8.96 (d, 2H), 7.09-7.07 (m, 1H), 6.73-6.41 (s, 2H), 4.75-4.65 (m, 2H), 4.61-4.52 (m, 2H), 4.30 (d, 2H), 3.59-3.41 (m, 1H), 3.05-2.84 (m, 2H), 2.56-2.52 (m, 2H) 1.54-1.44 (m, 6H).

### Example 121

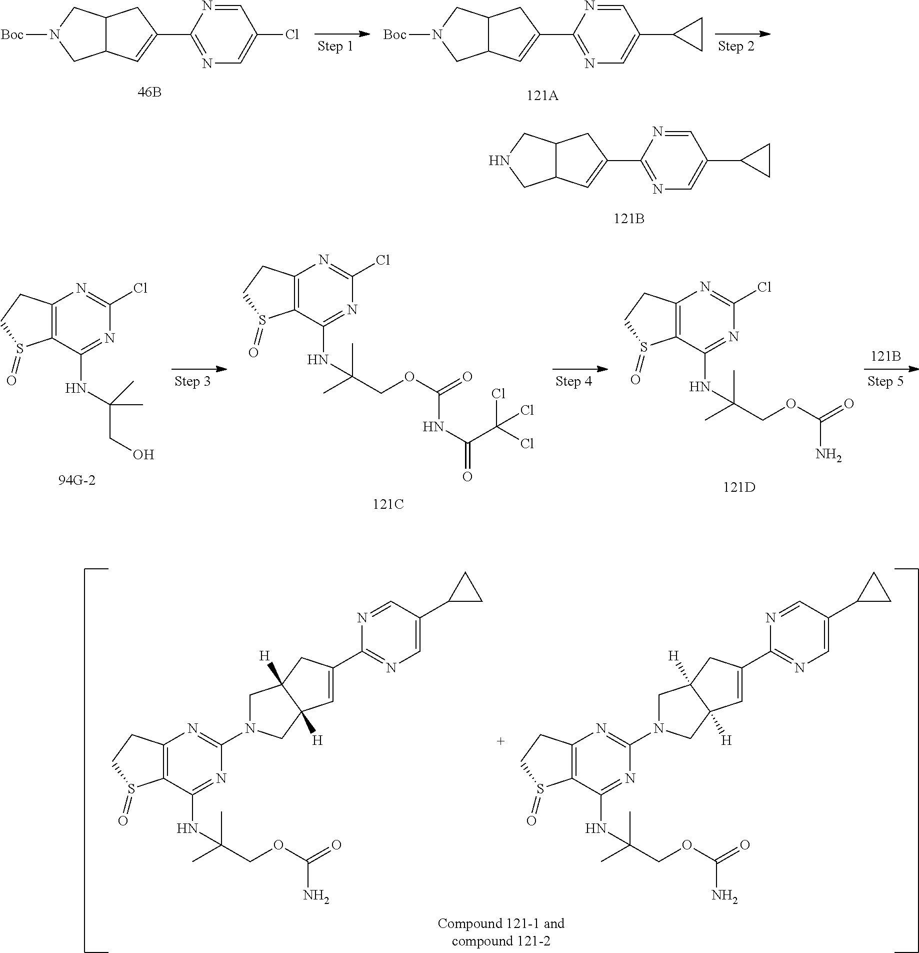

Using 46B as a raw material, compound 121 (300 mg, 96%) was obtained according to the operation of Example 48.

LC-MS (ESI): m/z=510.2 [M+H]⁺.

### Resolution of Compound 121:

Compound 121 (300 mg) was taken for resolution, and after separation, compound 121-1 (retention time: 0.568 min, 87.2 mg, ee %=100%) and compound 121-2 (retention time: 0.763 min, 84.1 mg, ee %=100%) were obtained.

### Resolution Conditions:

instrument: Waters 150 Prep-SFC F; column: Chiralcel AD column;
mobile phase: A for CO₂; B for 0.1% NH₃·H₂O in IPA and ACN; gradient: B 60%; flow: 100 mL/min; back pressure: 100 bar;
column temperature: 25° C.; wavelength: 220 nm; period: 3.2 min;

### Compound 121-1:

¹H NMR (400 MHz, DMSO-d6) δ=8.52 (s, 2H), 6.73 (s, 1H), 6.53 (s, 2H), 6.47 (s, 1H), 4.29 (s, 2H), 3.94-3.89 (m, 1H), 3.81-3.59 (m, 3H), 3.45-3.33 (m, 1H), 3.29-3.18 (m, 2H), 3.17-3.05 (m, 1H), 3.02-2.87 (m, 2H), 2.87-2.79 (m, 1H), 2.75 (d, 1H), 1.97-1.88 (m, 1H), 1.42 (s, 6H), 1.07-0.98 (m, 2H), 0.85-0.78 (m, 2H).

### Compound 121-2:

¹H NMR (400 MHz, DMSO-d6) δ=8.52 (s, 2H), 6.73 (s, 1H), 6.53 (s, 2H), 6.47 (s, 1H), 4.29-4.22 (m, 2H), 3.94-3.89 (m, 1H), 3.84-3.57 (m, 3H), 3.46-3.34 (m, 1H), 3.28-3.17 (m, 2H), 3.12 (s, 1H), 3.03-2.87 (m, 2H), 2.87-2.79 (m, 1H), 2.75 (d, 1H), 1.96-1.88 (m, 1H), 1.42 (s, 6H), 1.07-0.99 (m, 2H), 0.88-0.78 (m, 2H).

### Example 122

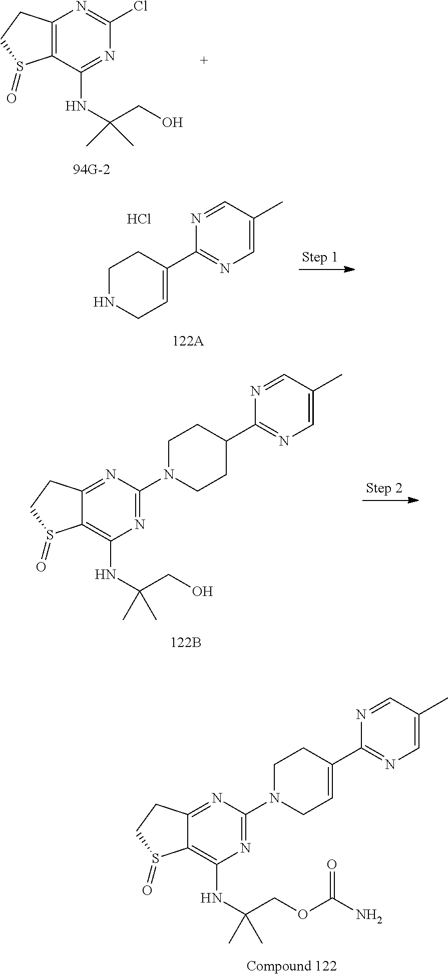 [SMILES: CC(C)(CO)Nc1nc(Cl)nc2c1[S@](=O)CC2 ; Cc1cnc(C2=CCNCC2)nc1 ; Cl ; Cc1cnc(C2CCN(c3nc4c(c(NC(C)(C)CO)n3)[S@](=O)CC4)CC2)nc1 ; Cc1cnc(C2=CCN(c3nc4c(c(NC(C)(C)COC(N)=O)n3)[S@](=O)CC4)CC2)nc1]

Using 94G-2 and 122A (synthesized according to WO 2017148518) as raw materials, the target compound 122 (26 mg, 12%) was obtained by preparation and purification by HPLC according to Example 114 (the operations of steps 4, 5, and 6).

LCMS (ESI): m/z=458.5 [M+H]⁺

Preparation method: instrument: SHIMADZU LC-20AP; preparative column: Phenomenex C18; mobile phase: A was 10 mM NH₄HCO₃ in H₂O; B was acetonitrile; gradient: phase B from 33 to 63 in 10 min; flow rate: 25 mL/min; column temperature: room temperature; detection wavelength: 210 nm;

¹H NMR (400 MHz, DMSO-d6) δ 8.63 (s, 2H), 7.23-7.19 (m, 1H), 6.70-6.45 (m, 2H), 4.47 (s, 2H), 4.30 (s, 2H), 4.00 (t, 2H), 3.47-3.38 (m, 1H), 3.35-3.22 (m, 2H), 3.01-2.83 (m, 2H), 2.68 (s, 2H), 2.26 (s, 3H), 1.46 (d, 6H).

### Example 123

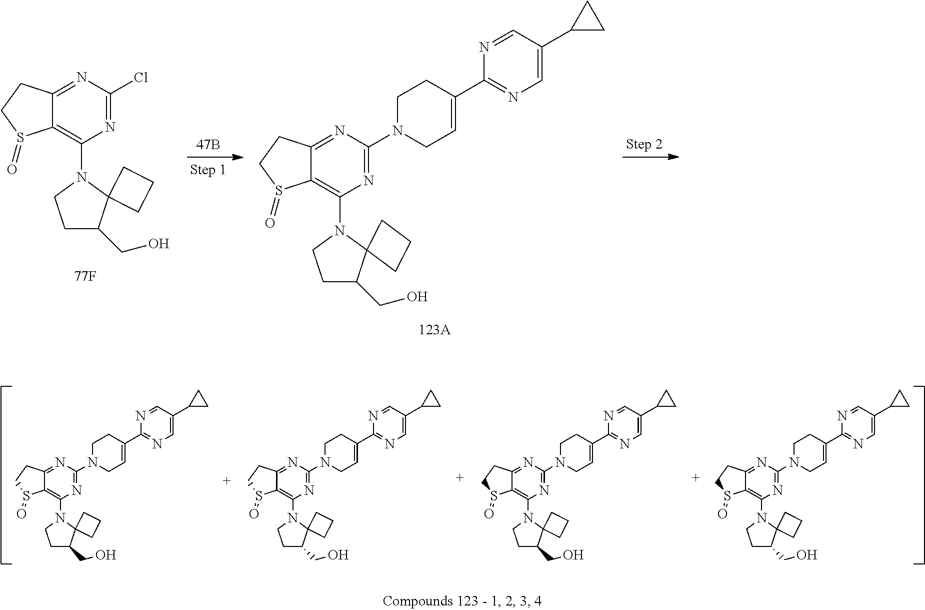

Using 77F and 47B as raw materials, compound 123-1 (70.9 mg), compound 123-2 (97.0 mg), compound 123-3 (58.4 mg), and compound 123-4 (35.0 mg) were obtained by chiral resolution according to Example 77 (steps 6 and 7).

HPLC analysis method: instrument: SHIMADZU LC-2020AD, column: C18 Column; mobile phase: A: 0.1% TFA in H₂O, B: ACN; gradient: 10-80% B in A; flow rate: 1.2 mL/min, column temperature: 45° C., wavelength: 210 nm & 254 nm.

HPLC preparation method: instrument: SHIMADZU LC-20AP, column: XB—SiOH Column; mobile phase: A: hexane, B: EtOH; gradient: 15-45% B gradient elution; flow rate: 90 mL/min, column temperature: 25° C.; wavelength: 220 nm; cycle time: 5.0 min, sample preparation: sample concentration 50 mg/ml, acetonitrile solution injection: 8.0 ml each time.

After separation, the fraction was dried by a rotary evaporator at a bath temperature of 35° C. to obtain compound P1 and compound P2.

SFC analysis method: instrument: SHIMADZU LC-30AD, column: Chiralcel AS Column; mobile phase: A:CO₂, B: 0.05% DEA in IPA and ACN; gradient: 60% B in A; flow rate: 3 mL/min, column temperature: 35° C., wavelength: 220 nm.

SFC preparation method: instrument: Waters 150 Prep-SFC E, column: Chiralcel Cellulose-2 Column; mobile phase: A: CO₂, B: 0.1% NH₃·H₂O in EtOH and ACN; gradient: 60% B gradient elution; flow rate: 100 mL/min, column temperature: 25° C.; wavelength: 220 nm; cycle time: 6.0 min; sample preparation: sample concentration 10 mg/ml, acetonitrile solution injection: 1.5 ml each time.

After separation, the fraction was dried by a rotary evaporator at a bath temperature of 35° C. to obtain compound 123-1 (70.9 mg), compound 123-2 (97.0 mg), compound 123-3 (58.4 mg), and compound 123-4 (35.0 mg).

Compound 123-1: retention time: 2.018 min; ¹H NMR (400 MHz, CDCl₃) δ 8.43 (s, 2H), 7.24-7.22 (m, 1H), 4.61-4.60 (m, 2H), 4.16-4.10 (m, 3H), 4.03-3.98 (m, 1H), 3.89-3.83 (m, 1H), 3.81-3.71 (m, 2H), 3.58-3.50 (m, 1H), 3.15-3.03 (m, 3H), 2.82-2.81 (m, 2H), 2.36-2.30 (m, 1H), 2.19-2.12 (m, 1H), 2.04-1.95 (m, 2H), 1.91-1.84 (m, 3H), 1.82-1.72 (m, 2H), 1.61 (s, 1H), 1.11-1.06 (m, 2H), 0.79-0.75 (m, 2H).

LC-MS (ESI): m/z=493.3 [M+H]⁺

Compound 123-2: retention time: 2.062 min; ¹H NMR (400 MHz, CDCl₃) δ 8.43 (s, 2H), 7.24-7.22 (m, 1H), 4.61-4.60 (m, 2H), 4.16-4.10 (m, 3H), 4.03-3.98 (m, 1H), 3.89-3.83 (m, 1H), 3.81-3.71 (m, 2H), 3.58-3.50 (m, 1H), 3.12-3.05 (m, 3H), 2.82-2.81 (m, 2H), 2.36-2.30 (m, 1H), 2.19-2.12 (m, 1H), 2.04-1.95 (m, 2H), 1.91-1.84 (m, 3H), 1.82-1.72 (m, 2H), 1.61 (s, 1H), 1.11-1.06 (m, 2H), 0.79-0.75 (m, 2H).

LC-MS (ESI): m/z=493.3 [M+H]⁺

Compound 123-3: retention time: 2.061 min; ¹H NMR (400 MHz, CDCl₃) δ 8.43 (s, 2H), 7.23-7.22 (m, 1H), 4.61-4.60 (m, 2H), 4.16-4.10 (m, 3H), 4.03-3.97 (m, 1H), 3.89-3.83 (m, 1H), 3.81-3.71 (m, 3H), 3.58-3.50 (m, 1H), 3.15-3.05 (m, 3H), 2.82 (s, 2H), 2.36-2.30 (m, 1H), 2.19-2.12 (m, 1H), 2.04-1.95 (m, 2H), 1.92-1.82 (m, 3H), 1.80-1.73 (m, 2H), 1.11-1.06 (m, 2H), 0.79-0.75 (m, 2H).

LC-MS (ESI): m/z=493.3 [M+H]⁺

Compound 123-4: retention time: 2.021 min; ¹H NMR (400 MHz, CDCl₃) δ 8.43 (s, 2H), 7.23-7.22 (m, 1H), 4.61-4.60 (m, 2H), 4.16-4.10 (m, 3H), 4.03-3.97 (m, 1H), 3.89-3.83 (m, 1H), 3.81-3.71 (m, 3H), 3.58-3.50 (m, 1H), 3.15-3.05 (m, 3H), 2.82 (s, 2H), 2.36-2.30 (m, 1H), 2.19-2.12 (m, 1H), 2.04-1.95 (m, 2H), 1.92-1.82 (m, 3H), 1.80-1.75 (m, 2H), 1.11-1.06 (m, 2H), 0.79-0.75 (m, 2H).

LC-MS (ESI): m/z=493.3 [M+H]⁺

### Example 124

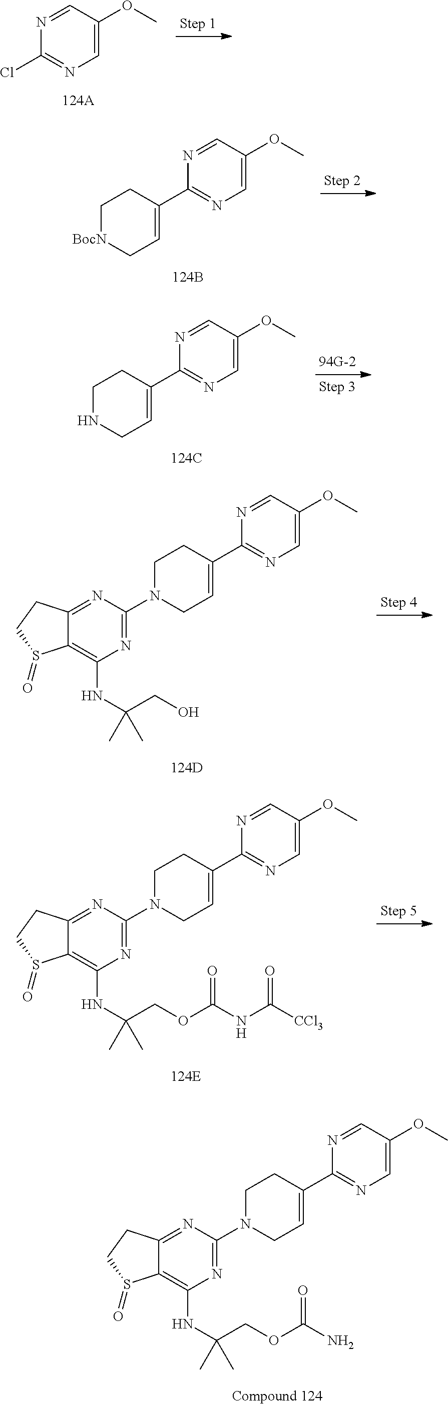

Using 124A as a raw material, the target compound 124 (80 mg, 18.18%) was obtained according to the operation of Example 119.

¹H NMR (400 MHz, CDCl₃) δ 8.37 (s, 2H), 7.12-7.10 (m, 1H), 5.48 (s, 1H), 4.51 (brs, 2H), 4.41-4.30 (m, 2H), 4.08-4.07 (m, 2H), 3.92 (s, 3H), 3.64-3.56 (m, 1H), 3.47 (s, 2H), 3.42-3.35 (m, 1H), 3.04-2.97 (m, 2H), 2.79 (s, 2H), 1.55 (s, 3H), 1.52 (s, 3H).

LC-MS (ESI): m/z=474.8 [M+H]⁺

### Example 125

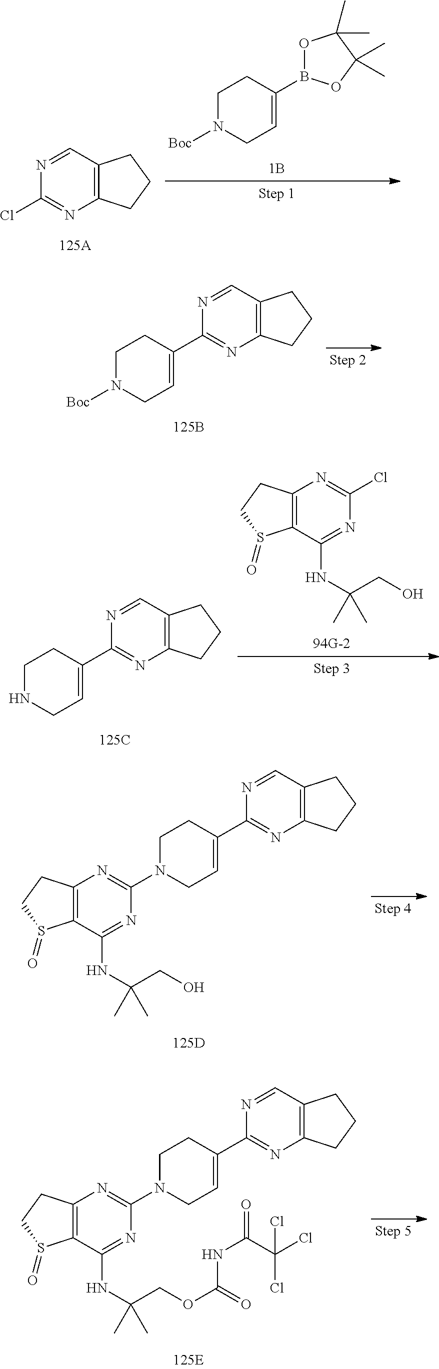

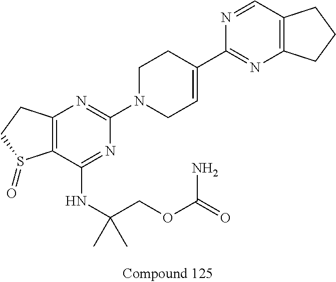 [SMILES: CC(C)(COC(N)=O)Nc1nc(N2CC=C(c3ncc4c(n3)CCC4)CC2)nc2c1[S@](=O)CC2]

Using 125A as a raw material, the target compound 125 (52 mg, 17%) was obtained according to the operation of Example 119.

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A with a content of 40-80%; c. flow: 15 mL/min; d. elution time: 20 min.

LCMS m/z=484.2 [M+1]⁺

¹H NMR (400 MHZ, DMSO-d6) δ 8.57 (s, 1H), 7.21 (s, 1H), 6.64 (s, 1H), 6.58 (s, 2H), 4.46 (s, 2H), 4.30 (s, 2H), 4.00 (t, 2H), 3.39-3.51 (m, 1H), 3.23-3.28 (m, 1H), 2.86-3.02 (m, 6H), 2.69 (s, 2H), 2.03-2.10 (m, 2H), 1.46 (d, 6H).

### Example 126

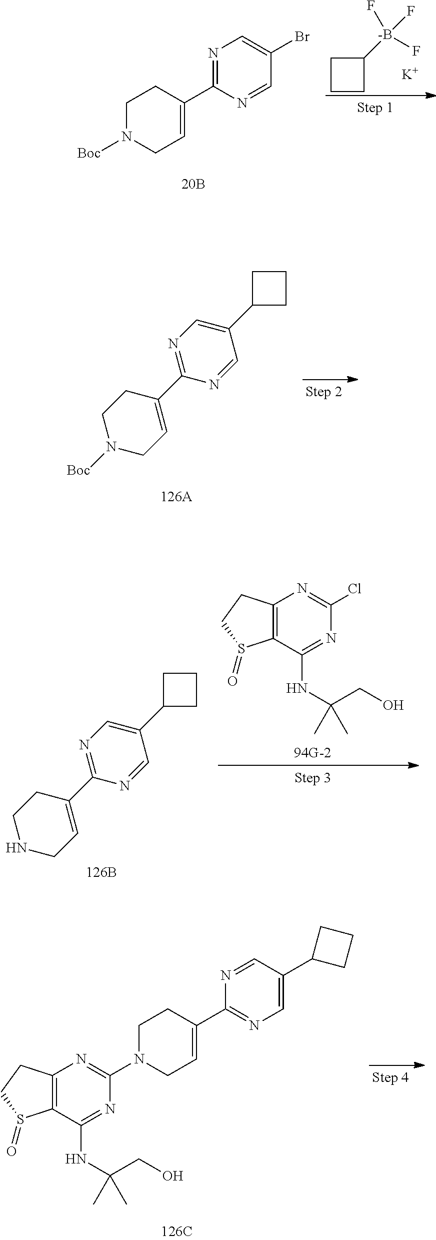

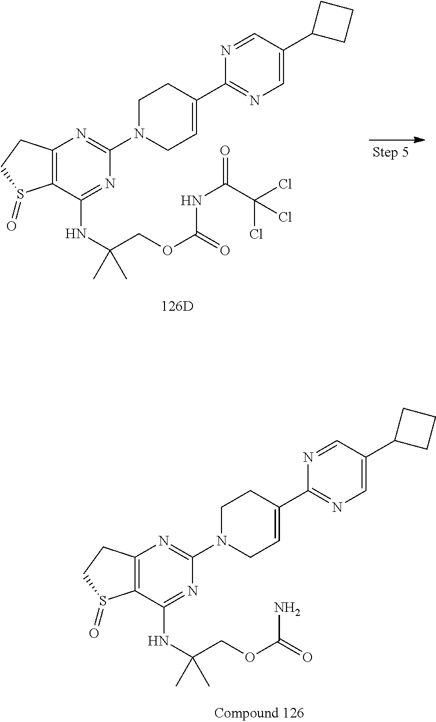 [SMILES: CC(C)(COC(=O)NC(=O)C(Cl)(Cl)Cl)Nc1nc(N2CC=C(c3ncc(C4CCC4)cn3)CC2)nc2c1[S@](=O)CC2 ; CC(C)(COC(N)=O)Nc1nc(N2CC=C(c3ncc(C4CCC4)cn3)CC2)nc2c1[S@](=O)CC2]

Using 20B and potassium cyclobutyltrifluoroborate as raw materials, the title compound 126 (100 mg, 32%) was obtained according to the operation of Example 116.

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A with a content of 40-80%; c. flow: 15 mL/min; d. elution time: 20 min.

LCMS m/z=498.2 [M+1]⁺

Compound 126, ¹H NMR (400 MHZ, DMSO-d6) δ 8.69 (s, 2H), 7.23 (s, 1H), 6.64 (s, 1H), 6.59 (s, 2H), 4.47 (s, 2H), 4.30 (s, 2H), 4.00 (t, 2H), 3.50-3.59 (m, 1H), 3.39-3.47 (m, 1H), 3.23-3.28 (m, 1H), 2.84-3.00 (m, 2H), 2.69 (s, 2H), 2.28-2.35 (m, 2H), 2.13-2.23 (m, 2H), 1.97-2.06 (m, 1H), 1.83-1.91 (m, 1H), 1.46 (d, 6H).

### Example 127

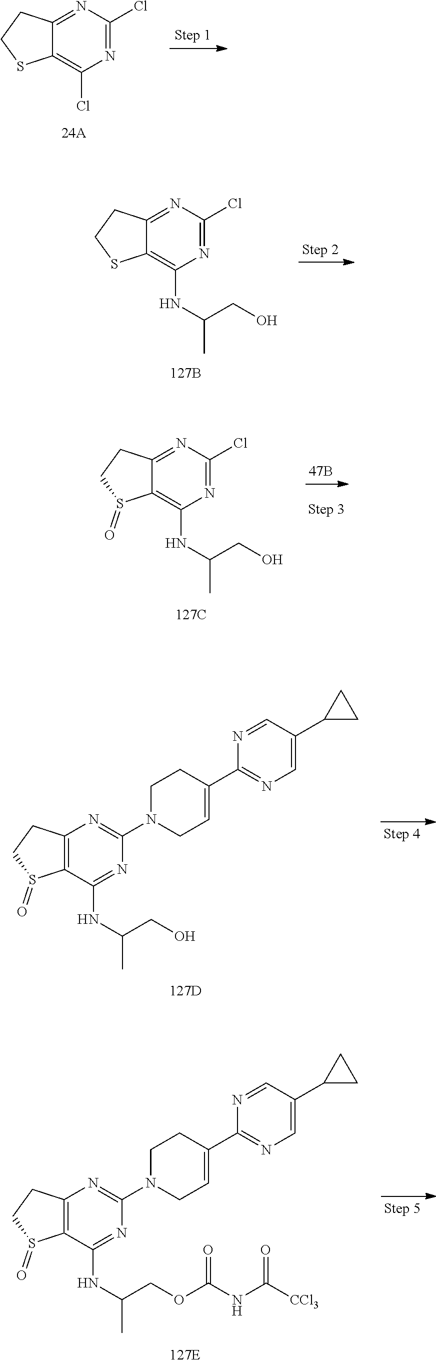

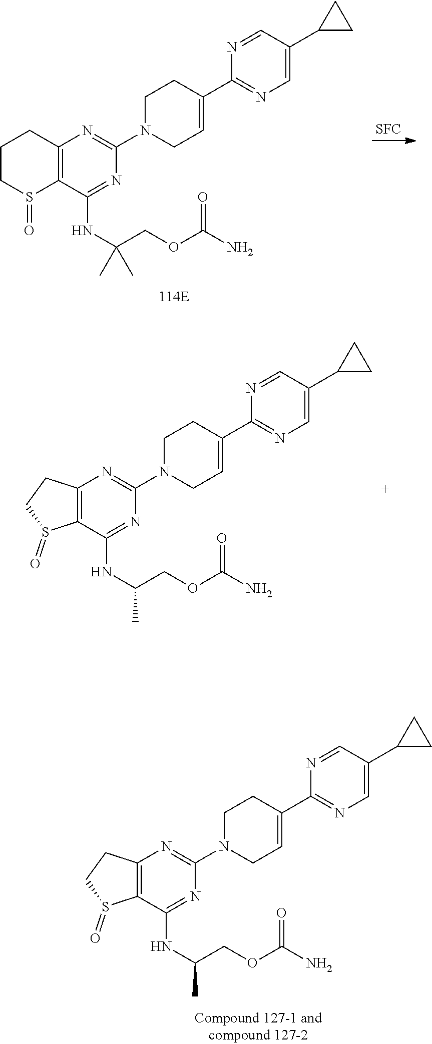 [SMILES: CC(C)(COC(N)=O)Nc1nc(N2CC=C(c3ncc(C4CC4)cn3)CC2)nc2c1S(=O)CCC2 ; "SFC" ; C[C@@H](COC(N)=O)Nc1nc(N2CC=C(c3ncc(C4CC4)cn3)CC2)nc2c1[S@](=O)CC2 ; C[C@H](COC(N)=O)Nc1nc(N2CC=C(c3ncc(C4CC4)cn3)CC2)nc2c1[S@](=O)CC2]

Using 24A and 2-aminopropanol as raw materials, compounds 127-1 (0.9 g, 20%, Rf 0.6) and 127-2 (1.8 g, 41%, Rf 0.3) were obtained by separation and purification through silica gel column chromatography (dichloromethane/methanol=10/1) according to the operation of Example 114.

LC-MS (ESI): m/z=470.2 [M+H]⁺.

¹H NMR (400 MHz, DMSO-d₆) δ 8.54 (s, 2H), 7.56 (d, 1H), 7.28-7.12 (m, 1H), 6.50 (s, 2H), 4.60-4.49 (m, 1H), 4.49-4.38 (m, 2H), 4.10-3.79 (m, 4H), 3.51-3.40 (m, 1H), 3.27-3.16 (m, 1H), 3.01-2.82 (m, 2H), 2.66 (s, 2H), 1.98-1.89 (m, 1H), 1.17 (d, 3H), 1.11-1.00 (m, 2H), 0.90-0.76 (m, 2H).

LC-MS (ESI): m/z=470.2 [M+H]⁺.

¹H NMR (400 MHz, DMSO-d₆) δ 8.54 (s, 2H), 7.50 (d, 1H), 7.21-7.15 (m, 1H), 6.47 (s, 2H), 4.65-4.49 (m, 1H), 4.49-4.38 (m, 2H), 4.09-3.87 (m, 4H), 3.51-3.39 (m, 1H), 3.26-3.15 (m, 1H), 3.05-2.83 (m, 2H), 2.66 (s, 2H), 1.98-1.89 (m, 1H), 1.19 (d, 3H), 1.08-0.95 (m, 2H), 0.91-0.80 (m, 2H).

### Example 128

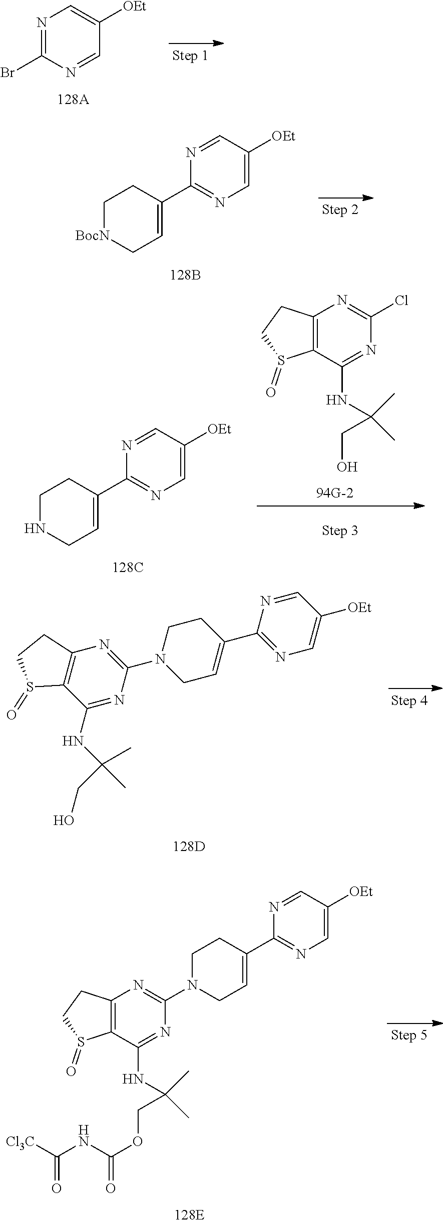

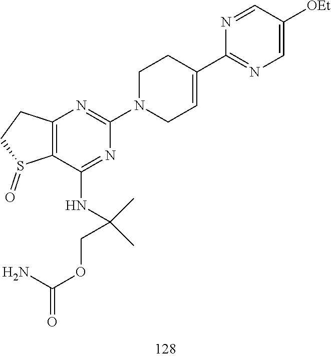 [SMILES: CCOc1cnc(C2=CCN(c3nc4c(c(NC(C)(C)COC(N)=O)n3)[S@](=O)CC4)CC2)nc1]

Using 128A as a raw material, the target compound 128 (490 mg, 41%) was obtained according to the operation of Example 119.

LC-MS (ESI): m/z=488.2 [M+H]⁺

¹H NMR (400 MHz, DMSO-d₆) δ 8.51 (s, 2H), 7.19-7.01 (m, 1H), 6.70-6.40 (m, 3H), 4.45 (d, 2H), 4.30 (s, 2H), 4.23-4.16 (m, 2H), 4.00 (t, 2H), 3.49-3.36 (m, 1H), 3.29-3.22 (m, 1H), 3.02-2.91 (m, 1H), 2.91-2.82 (m, 1H), 2.73-2.62 (m, 2H), 1.46 (d, 6H), 1.36 (t, 3H).

### Example 129

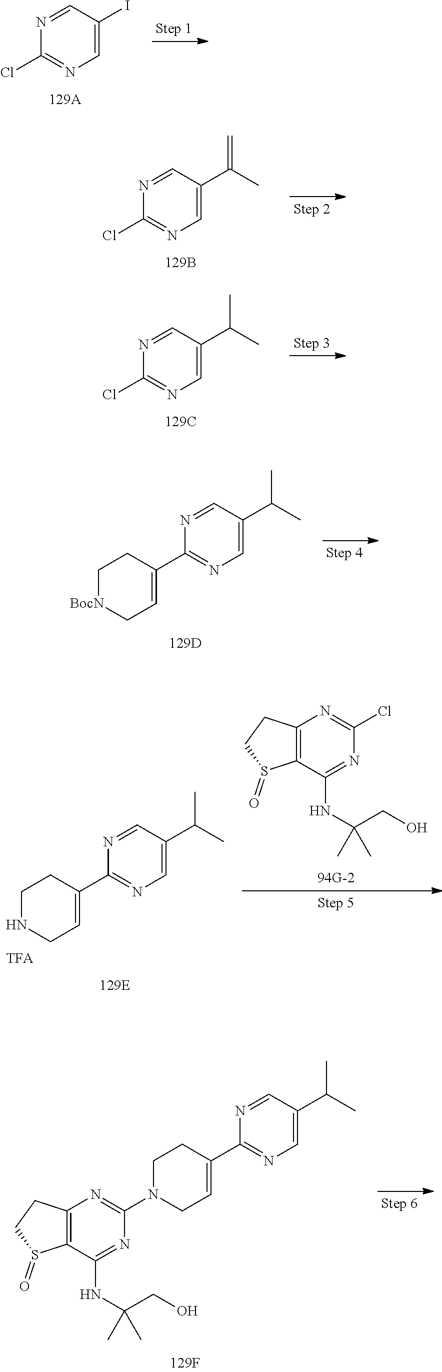

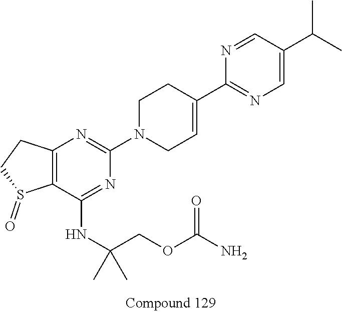 [SMILES: CC(C)c1cnc(C2=CCN(c3nc4c(c(NC(C)(C)COC(N)=O)n3)[S@](=O)CC4)CC2)nc1]

Step 1: 129A (2.4 g, 10 mmol) was dissolved in 1,4-dioxane (50 mL), and 1,1-bis(diphenylphosphino)ferrocene-palladium dichloride (0.73 g, 1 mmol), cesium carbonate (6.5 g, 20 mmol), isopropenylboronic acid pinacol ester (2.1 g, 12 mmol), and water (10 mL) were then added. After nitrogen displacement three times, the mixture was heated to 90° C. and reacted for 3 h. The system was then passed through a short silica gel column, eluted with ethyl acetate, concentrated, and subjected to column chromatography (petroleum ether/ethyl acetate=9/1) to obtain compound 129B (1.4 g, 90%).

LC-MS (ESI): m/z=155.6 [M+H]⁺%

Step 2: 129B (1.4 g, 9 mmol) was dissolved in anhydrous ethanol, and platinum dioxide (0.2 g, 0.9 mmol) was then added. After insertion of a hydrogen balloon for purging three times, the mixture was reacted for 12 h, then passed through a short silica gel column, eluted with ethyl acetate, concentrated, and subjected to column chromatography (petroleum ether/ethyl acetate=9/1) to obtain compound 129C (1.4 g, 98%).

¹H NMR (400 MHz, CDCl₃) δ 8.50 (s, 2H), 3.01-2.94 (m, 1H), 1.32 (d, 6H).

Using 129C as a raw material, compound 129 (650 mg, 48%) was obtained according to the operation of Example 49.

LC-MS (ESI): m/z=486.6 [M+H]⁺.

¹H NMR (400 MHz, DMSO-d6) δ 8.70 (s, 2H), 7.22 (s, 1H), 6.63 (s, 1H), 6.56 (s, 2H), 4.47 (s, 2H), 4.30 (s, 2H), 4.00 (t, 2H), 3.55-3.38 (m, 1H), 3.29-3.24 (m, 1H), 3.02-2.82 (m, 3H), 2.69 (s, 2H), 1.46 (d, 6H), 1.26 (d, 6H).

### Example 130

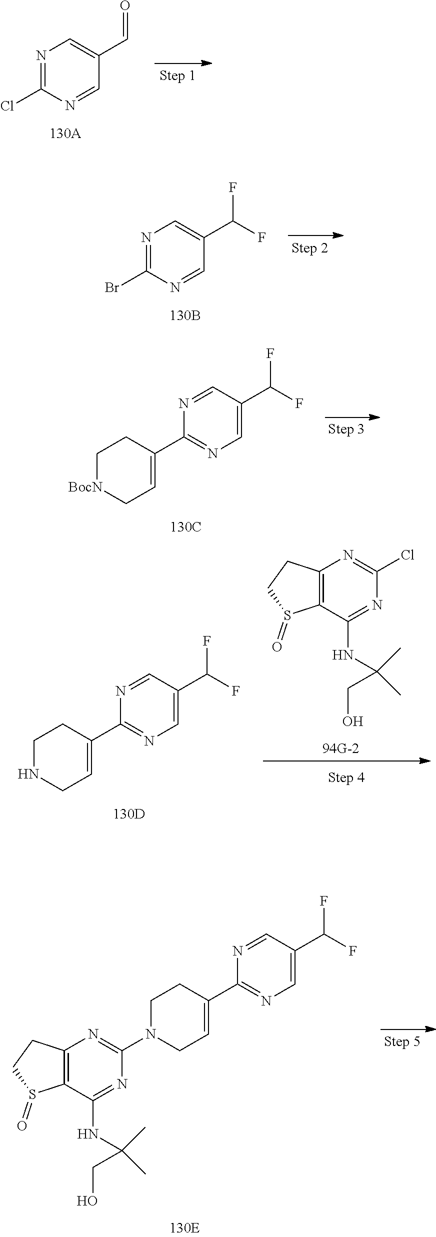 [SMILES: O=Cc1cnc(Cl)nc1 ; FC(F)c1cnc(Br)nc1 ; CC(C)(C)OC(=O)N1CC=C(c2ncc(C(F)F)cn2)CC1 ; FC(F)c1cnc(C2=CCNCC2)nc1 ; CC(C)(CO)Nc1nc(Cl)nc2c1[S@](=O)CC2 ; CC(C)(CO)Nc1nc(N2CC=C(c3ncc(C(F)F)cn3)CC2)nc2c1[S@](=O)CC2]

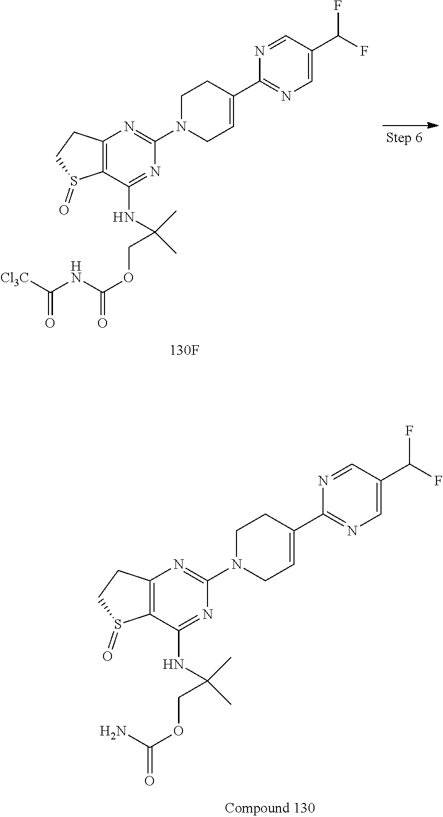

Step 1: Compound 130A (6.2 g, 43.5 mmol) was dissolved in dichloromethane (100 mL), and diethylaminosulfur trifluoride (14.1 g, 87.0 mmol) was then slowly added at −78° C. After the addition was complete, the mixture was slowly heated to room temperature and reacted overnight. The reaction was quenched by adding a sodium bicarbonate solution, and the system was extracted with dichloromethane, dried, filtered, concentrated, and subjected to column chromatography (petroleum ether/ethyl acetate=10/1) to obtain compound 130B (4.9 g, 68%).

Using 130B as a raw material, compound 130 (800 mg, 61%) was obtained according to the operation of Example 49.

LC-MS (ESI): m/z=494.1 [M+H]⁺.

¹H NMR (400 MHz, DMSO-d6) δ 9.01 (s, 2H), 7.41 (s, 1H), 7.19 (t, 1H), 6.60 (d, 3H), 4.52 (s, 2H), 4.31 (s, 2H), 4.02 (t, 2H), 3.52-3.36 (m, 1H), 3.28-3.21 (m, 1H), 3.07-2.78 (m, 2H), 2.72 (s, 2H), 1.47 (d, 6H).

¹⁹F NMR (376 MHz, DMSO-d6) δ −110.81 (s).

### Example 131

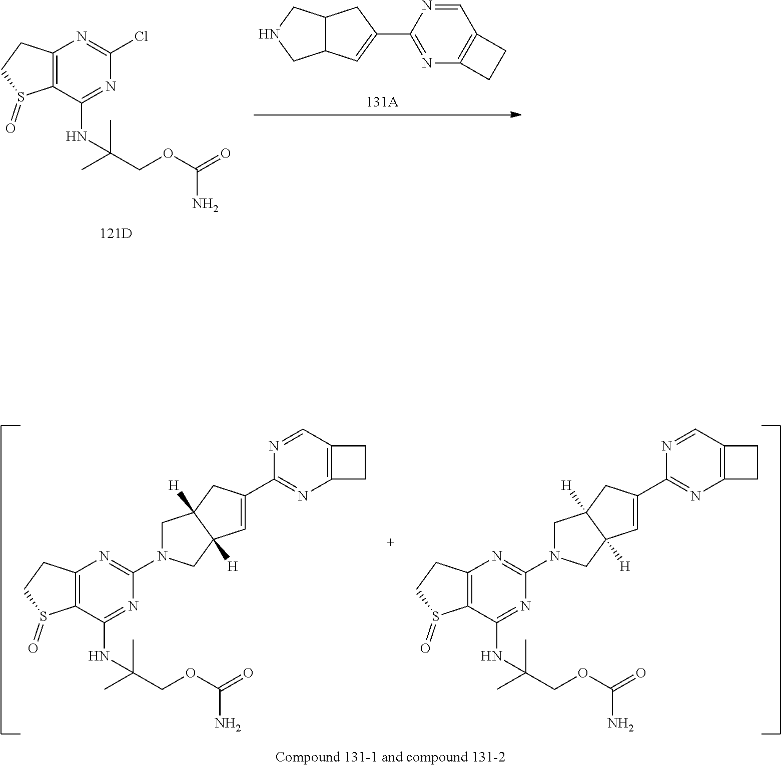

Compound 121D (149 mg, 0.47 mmol) was dissolved in 1,4-dioxane (10.0 mL), compound 131A (100 mg, 0.47 mmol) and diisopropylethylamine (182 mg, 1.41 mmol) were then added, and the mixture was heated back to 100° C. and reacted for 5 h. The system was then cooled to room temperature, concentrated, and subjected to column chromatography (dichloromethane/methanol=10/1) to obtain compound 131 (200 mg, 86%).

LC-MS (ESI): m/z=496.2 [M+H]⁺.

Resolution of compound 131: Compound 131 (200 mg) was taken for resolution, and after separation, compound 131-1 (retention time: 0.972 min, 48.9 mg, ee %=100%) and compound 131-2 (retention time: 1.852 min, 45.8 mg, ee %=100%) were obtained. Resolution conditions: instrument: Waters 150 Prep-SFC E; column: Chiralcel Cellulose-2 column; mobile phase: A for CO₂; B for 0.1% NH₃·H₂O in IPA and ACN; gradient: B 70%; flow: 100 mL/min; back pressure: 100 bar; column temperature: 25° C.; wavelength: 220 nm; period: 7.0 min;

### Compound 131-1:

¹H NMR (400 MHz, DMSO-d6) δ=8.32 (s, 1H), 6.75 (s, 1H), 6.52 (s, 2H), 4.36-4.18 (m, 2H), 3.94-3.89 (m, 1H), 3.82-3.60 (m, 3H), 3.45-3.36 (m, 3H), 3.28-3.23 (m, 2H), 3.21-3.19 (m, 3H), 3.15-3.07 (m, 1H), 3.01-2.88 (m, 2H), 2.87-2.81 (m, 1H), 2.74 (d, 1H), 1.44 (s, 6H).

### Compound 131-2:

¹H NMR (400 MHz, DMSO-d6) δ=8.32 (s, 1H), 6.75 (s, 1H), 6.54 (s, 1H), 6.47 (s, 1H), 4.33-4.17 (m, 2H), 3.94-3.89 (m, 1H), 3.81-3.60 (m, 3H), 3.41-3.35 (m, 3H), 3.28-3.23 (m, 2H), 3.23-3.16 (m, 3H), 3.16-3.05 (m, 1H), 3.02-2.88 (m, 2H), 2.87-2.79 (m, 1H), 2.74 (d, 1H), 1.43 (s, 6H).

### Example 132

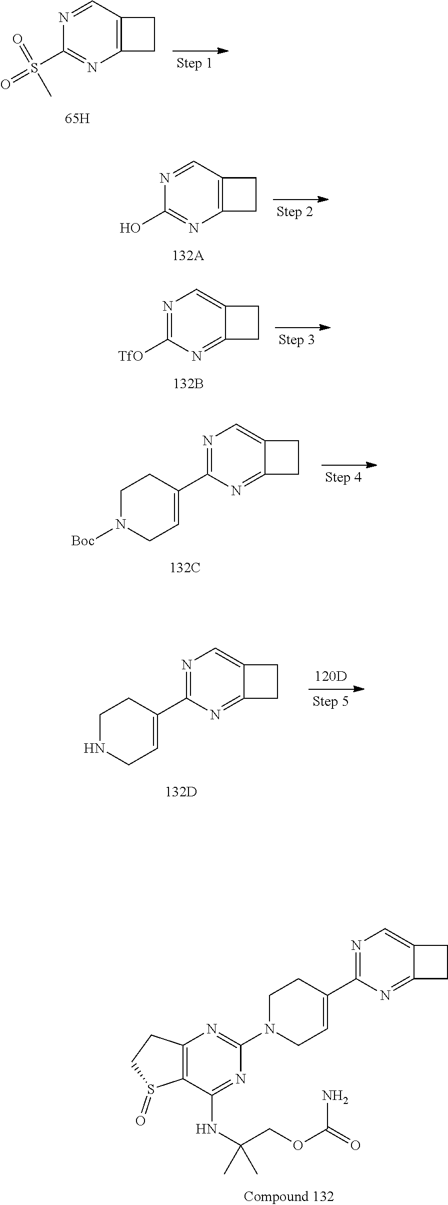 [SMILES: CS(=O)(=O)c1ncc2c(n1)CC2 ; Oc1ncc2c(n1)CC2 ; O=S(=O)(Oc1ncc2c(n1)CC2)C(F)(F)F ; CC(C)(C)OC(=O)N1CC=C(c2ncc3c(n2)CC3)CC1 ; C1=C(c2ncc3c(n2)CC3)CCNC1 ; CC(C)(COC(N)=O)Nc1nc(N2CC=C(c3ncc4c(n3)CC4)CC2)nc2c1[S@](=O)CC2]

Step 1: 65H (500 mg, 2.7 mmol) was dissolved in tetrahydrofuran (20 ml), sodium hydroxide (216 mg, 5.4 mmol) dissolved in water (0.5 ml) was dropwise added, and the mixture was heated to 60° C. and reacted for 1 h. The system was then concentrated and quickly separated and purified (methanol/dichloromethane=0-10%) to obtain compound 132A (298 mg, 90%).

LC-MS (ESI): m/z=123.1 [M+H]⁺

Step 2: Compound 132A (298 mg, 2.44 mmol) was dissolved in N,N-dimethylformamide (5 ml), and N-Phenyl-bis(trifluoromethanesulfonyl)imide (1.74 g, 4.88 mmol) and potassium carbonate (1.01 g, 7.32 mmol) were added and stirred at room temperature 3 h. After the reaction was complete, the reaction system was diluted by adding ethyl acetate (40 ml). The organic phase was washed with water (10 ml*3), and the organic phases were dried over anhydrous sodium sulfate, concentrated, and quickly separated and purified (ethyl acetate/petroleum ether=0-20%) to obtain compound 132B (589 mg, 95%).

LC-MS (ESI): m/z=255.1 [M+H]⁺

Step 3: Compound 132B (100 mg, 0.39 mmol) and tert-butyl 4-(4,4,5,5-tetramethyl-1,3,2-dioxaborolan-2-yl)-3,6-dihydropyridine-1(2H)-carboxylate (183 mg, 0.59 mmol) were dissolved in 1,4-dioxane/water (5 ml/1 ml), and Pd (dppf) Cl₂ (29 mg, 0.04 mmol) and cesium carbonate (381 mg, 1.17 mmol) were added. After nitrogen displacement three times, the mixture was heated to 100° C. and reacted for 3 h, and the system was then concentrated and quickly separated and purified (petroleum ether/ethyl acetate=10/1) to obtain compound 132C (101 mg, 90%).

LC-MS (ESI): m/z=288.2 [M+H]⁺%

Step 4: Compound 132C (50 mg, 0.18 mmol) was dissolved in dichloromethane/trifluoroacetic acid (6 mL/2 ml) and reacted at room temperature for half an hour, and the system was then concentrated to obtain compound 132D, which was directly used for the next step.

LC-MS (ESI): m/z=188.2 [M+H]⁺.

Step 5: The compound 132D from the above step was dissolved in 1,4-dioxane (10.0 mL), and diisopropylethylamine (70 mg, 0.54 mmol) and compound 120D (55 mg, 0.18 mmol) were then added, heated back to 100° C., and reacted for 5 h, and the system was then cooled to room temperature, concentrated, and quickly separated and purified (dichloromethane/methanol=10/1) to obtain compound 132 (30 mg, 36%).

¹H NMR (400 MHz, DMSO-d6) δ=8.35 (s, 1H), 7.21 (s, 1H), 6.64 (s, 1H), 6.56 (s, 1H), 4.46 (s, 2H), 4.30 (s, 2H), 3.99 (t, J=5.6, 2H), 3.49-3.36 (m, 3H), 3.29-3.24 (m, 1H), 3.24-3.18 (m, 2H), 3.02-2.92 (m, 1H), 2.88-2.84 (m, 1H), 2.67 (s, 2H), 1.46 (d, 6H).

LC-MS (ESI): m/z=470.2 [M+H]⁺.

### Example 133

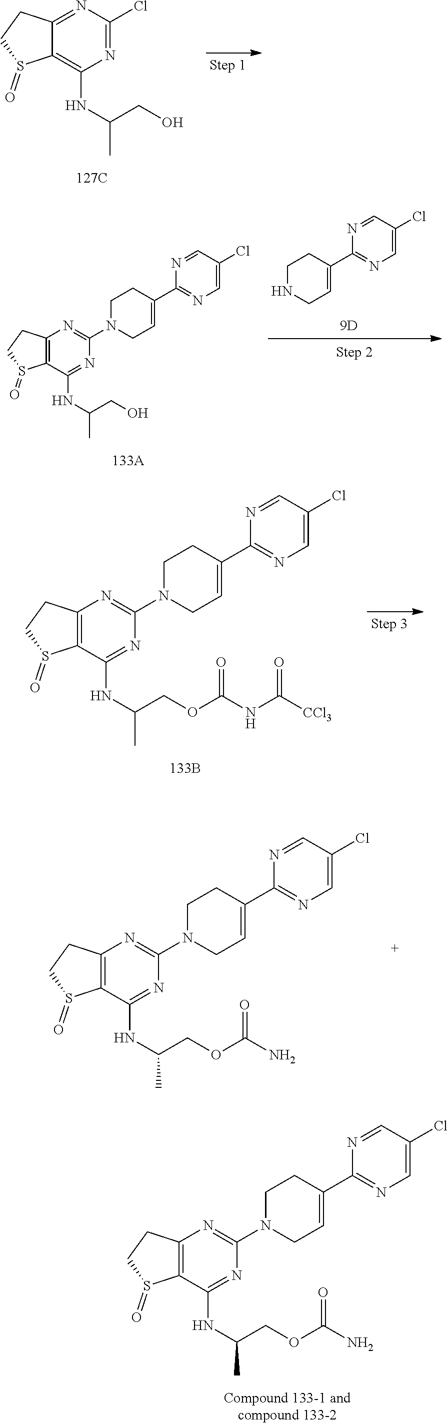

Using 127C as a raw material, compounds 133-1 (241 mg, 6%) and 133-2 (120 mg, 3%) were obtained by resolution according to the synthesis method in Example 127. Preparation method: instrument: WATYERS 150 preparative SFC (SFC-26), column: ChiralPak AD column (250 mm*30 mm, 10 μm), mobile phase: (phase A: CO₂, phase B: IPA (0.1% NH₃·H₂O)); gradient: 30% mobile phase B, isocratic elution; flow rate: 150 mL/min, back pressure: 100 bar, column temperature: 38° C., wavelength: 220 nm, cycle time: 30 min, sample preparation: sample concentration 5 mg/ml, methanol/DCM solution injection: 4.0 ml each time. After separation, the fraction was dried by a rotary evaporator in a water bath at 40° C. to obtain products 133-1 (retention time: 1.934 min) and 133-2 (retention time: 2.424 min).

133-1: LC-MS (ESI): m/z=464.1 [M+H]⁺.

¹H NMR (400 MHz, DMSO-d₆) δ 8.90 (s, 2H), 7.51 (d, 1H), 7.40-7.17 (m, 1H), 6.46 (s, 2H), 4.63-4.42 (m, 3H), 4.10-3.86 (m, 4H), 3.50-3.38 (m, 1H), 3.26-3.20 (m, 1H), 3.05-2.93 (m, 1H), 2.92-2.83 (m, 1H), 2.73-2.61 (m, 2H), 1.19 (d, 3H).

133-2: LC-MS (ESI): m/z=464.1 [M+H]⁺.

¹H NMR (400 MHz, DMSO-d₆) δ 8.90 (s, 2H), 7.57 (d, 1H), 7.34-7.22 (m, 1H), 6.50 (s, 2H), 4.63-4.42 (m, 3H), 4.10-3.86 (m, 4H), 3.52-3.40 (m, 1H), 3.26-3.18 (m, 1H), 3.05-2.93 (m, 1H), 2.92-2.83 (m, 1H), 2.71-2.61 (m, 2H), 1.17 (d, 3H).

### Example 134

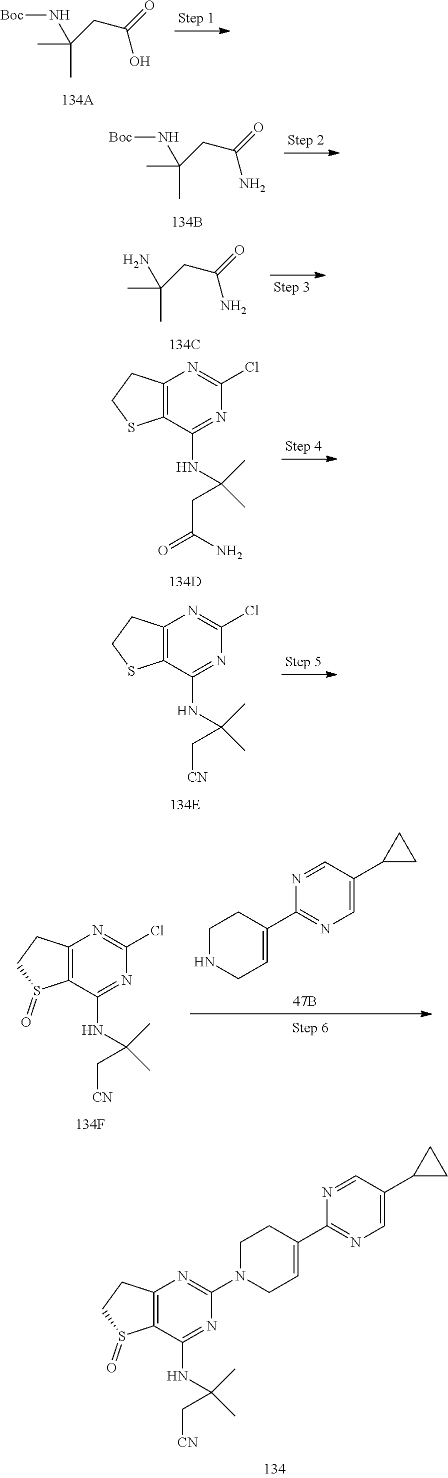

Using 134A as a raw material, compound 134 (315 mg, 50%) was obtained according to the operation of Example 83. Preparation method: instrument: Waters 150 Prep-SFC A, column: Chiralcel AD column (250 mm×30 mm, 10 μm), mobile phase: (phase A: CO₂, phase B: IPA and ACN (0.1% NH₃·H₂O)); gradient: 70% mobile phase B, isocratic elution; flow rate: 100 mL/min, back pressure: 100 bar, column temperature: 25° C., wavelength: 220 nm, cycle time: 5.3 min, sample preparation: sample concentration 10 mg/ml, acetonitrile solution injection: 2.5 ml each time. After separation, the fraction was dried in a water bath at 35° C. by a rotary evaporator to obtain a product. The solvent was then dried with a freeze-dryer at −80° C. to obtain the final product 134 (retention time: 0.828 min).

LC-MS (ESI): m/z=450.3 [M+H]⁺

¹H NMR (400 MHz, DMSO-d₆) δ 8.55 (s, 2H), 7.29-7.15 (m, 1H), 7.04 (s, 1H), 4.45 (s, 2H), 3.98 (t, 2H), 3.51-3.40 (m, 1H), 3.35 (d, 1H), 3.28-3.21 (m, 2H), 3.06-2.94 (m, 1H), 2.94-2.84 (m, 1H), 2.68 (s, 2H), 2.02-1.90 (m, 1H), 1.54 (d, 6H), 1.09-0.98 (m, 2H), 0.92-0.78 (m, 2H).

### Example 135

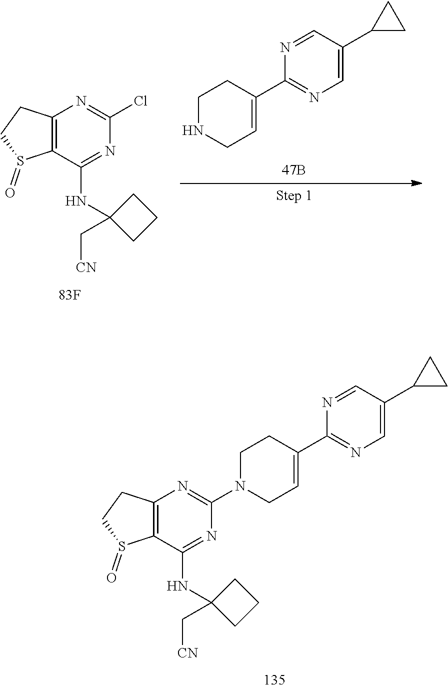 [SMILES: N#CCC1(Nc2nc(Cl)nc3c2[S@](=O)CC3)CCC1 ; C1=C(c2ncc(C3CC3)cn2)CCNC1 ; N#CCC1(Nc2nc(N3CC=C(c4ncc(C5CC5)cn4)CC3)nc3c2[S@](=O)CC3)CCC1]

Step 1: Compound 83F (500 mg, 1.68 mmol) was dissolved in 1,4-dioxane (20 mL), and compound 47B (410 mg, 2.02 mmol) and N,N-diisopropylethylamine (1.08 g, 8.39 mmol) were then added and stirred at 90 degrees Celsius overnight under nitrogen protection. After the reaction was complete, the reaction liquid was concentrated and subjected to silica gel column chromatography (dichloromethane/methanol=10/1), and the crude product was subjected to SFC chiral resolution to obtain compound 135 (608 mg, 78%).

Preparation method: instrument: Waters 150 Prep-SFC F, column: Chiralcel AD column (250 mm*30 mm, 10 μm), mobile phase: (phase A: CO₂, phase B: IPA and ACN (0.1% NH₃·H₂O)); gradient: 70% mobile phase B, isocratic elution; flow rate: 100 mL/min, back pressure: 100 bar, column temperature: 25° C., wavelength: 220 nm, cycle time: 4.3 min, sample preparation: sample concentration 5 mg/ml, acetonitrile solution injection: 2.0 ml each time. After separation, the fraction was dried in a water bath at 35° C. by a rotary evaporator to obtain a product. The solvent was then dried with a freeze-dryer at −80° C. to obtain the final product 135 (retention time: 0.956 min).

LC-MS (ESI): m/z=462.2 [M+H]⁺

¹H NMR (400 MHz, DMSO-d6) δ 8.54 (s, 2H), 8.14 (s, 1H), 7.20-7.16 (m, 1H), 4.42 (s, 2H), 3.97 (d, 2H), 3.52-3.40 (m, 1H), 3.36 (d, 2H), 3.28-3.17 (m, 1H), 3.03-2.94 (m, 1H), 2.93-2.85 (m, 1H), 2.75-2.61 (m, 2H), 2.48-2.33 (m, 2H), 2.32-2.20 (m, 2H), 2.02-1.79 (m, 3H), 1.09-0.93 (m, 2H), 0.88-0.76 (m, 2H).

### Example 136

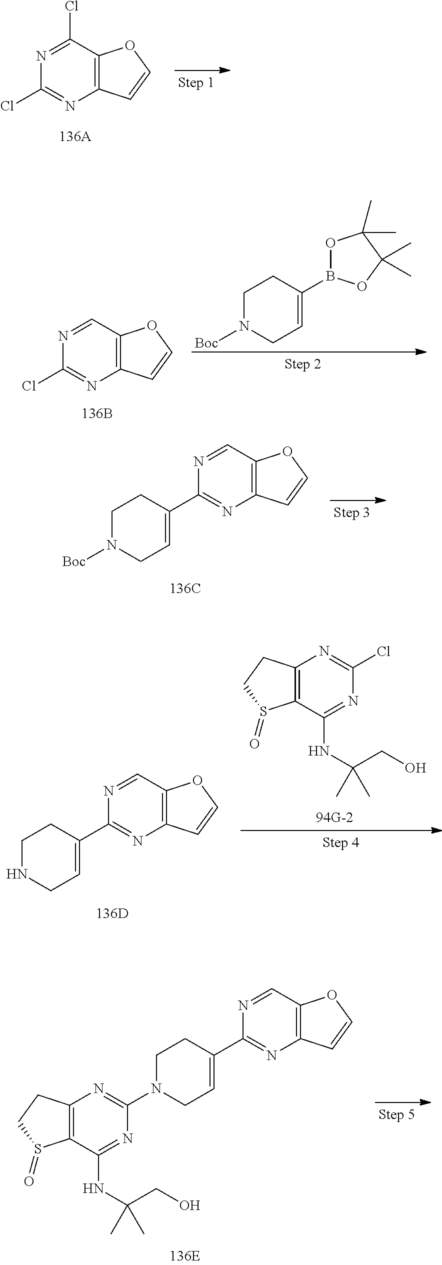

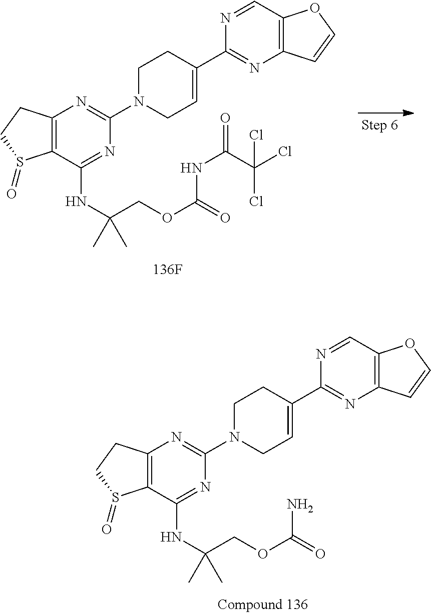 [SMILES: CC(C)(COC(=O)NC(=O)C(Cl)(Cl)Cl)Nc1nc(N2CC=C(c3ncc4occc4n3)CC2)nc2c1[S@](=O)CC2 ; CC(C)(COC(N)=O)Nc1nc(N2CC=C(c3ncc4occc4n3)CC2)nc2c1[S@](=O)CC2]

Step 1: Compound 136A (5.00 g, 26.49 mmol) and acetic acid (9.54 g, 158.94 mmol) were weighed into a 100 mL single-mouth flask and dissolved with methanol (50 mL), and zinc powder (6.93 g, 105.96 mmol) was added in portions. After the addition was complete, the mixture was stirred at 70 degrees Celsius for 0.5 h. After the reaction was complete as detected by a TLC spotting plate (petroleum ether:ethyl acetate=5:1), water (50 mL) was added and stirred for 5 min, and the mixture was extracted with ethyl acetate (50 mL). The organic phase was separated, dried over anhydrous sodium sulfate, and concentrated under reduced pressure, and the crude product was purified by column chromatography (eluents: petroleum ether:ethyl acetate=5:1) to obtain the target compound 136B (1.0 g, yield: 24%).

LCMS m/z=155.1 [M+1]⁺

Using compound 136B as a raw material, the title compound 136 (90 mg, 23%) was obtained according to the operation of Example 119.

Preparation method: instrument: waters 2767 preparative liquid chromatographic instrument; chromatographic column: SunFire® Prep C18 (19 mm×250 mm); the sample was dissolved with DMF and filtered with a 0.45 μm filter to prepare a sample liquid; preparative chromatography conditions: a. composition of mobile phases A and B: mobile phase A: acetonitrile, and mobile phase B: water (containing 5 mM aqueous ammonia); b. gradient elution, mobile phase A with a content of 30-70%; c. flow: 15 mL/min; d. elution time: 20 min.

LCMS m/z=484.2 [M+1]⁺

Compound 136, ¹H NMR (400 MHZ, DMSO-d6) δ 9.13 (s, 1H), 8.52 (d, 1H), 7.26 (s, 1H), 7.22 (d, 1H), 6.63 (s, 1H), 6.55 (s, 2H), 4.49 (s, 2H), 4.31 (s, 2H), 4.03 (t, 2H), 3.40-3.51 (m, 1H), 3.24-3.31 (m, 1H), 2.84-3.01 (m, 2H), 2.78 (s, 2H), 1.47 (d, 6H).

### Example 137

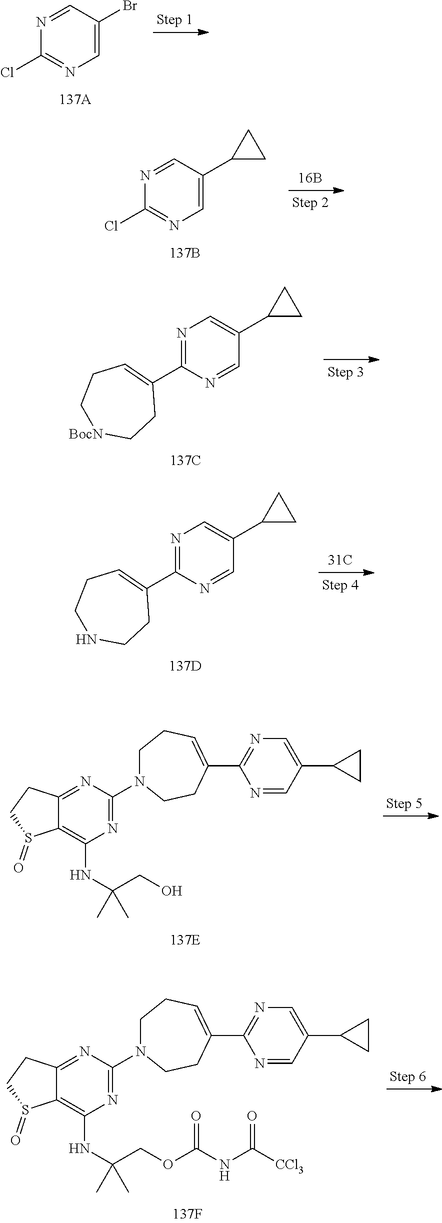

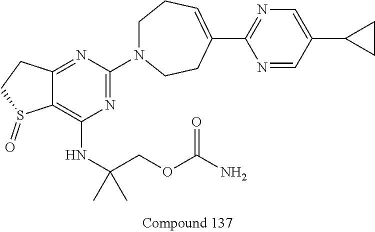 [SMILES: CC(C)(COC(N)=O)Nc1nc(N2CCC=C(c3ncc(C4CC4)cn3)CC2)nc2c1[S@](=O)CC2]

Step 1: Compound 137A (2.00 g, 10.34 mmol) was dissolved in tetrahydrofuran (40 mL), and potassium carbonate (3.57 g, 25.85 mmol), cyclopropylboronic acid (0.98 g, 11.37 mmol), and water (8 mL) were then successively added. After nitrogen displacement three times, 1,1-bis(diphenylphosphino)ferrocene-palladium dichloride (0.92 g, 1.24 mmol) was added. After the addition was complete, the reaction was heated to 65° C. and stirred for 16 h. After the reaction liquid naturally returned to room temperature, the reaction was diluted by adding water (50 mL) and extracted with ethyl acetate (50 mL) three times. The organic phases were combined, dried over anhydrous sodium sulfate, and then concentrated in vacuum to obtain a crude product, which was purified by column chromatography (eluents: petroleum ether/ethyl acetate=4/1) to obtain compound 137B (1.00 g, 62.56%).

¹H NMR (400 MHz, CDCl₃) 8.35 (s, 2H), 1.90-1.83 (m, 1H), 1.16-1.11 (m, 2H), 0.81-0.77 (m, 2H).

LC-MS (ESI): m/z=155.1 [M+H]⁺.

Using 137B as a raw material, the target compound 137 (48 mg, 13.20%) was obtained according to the operation of Example 119.

¹H NMR (400 MHz, CDCl₃) δ 8.38 (s, 2H), 7.35-7.28 (m, 1H), 5.34 (s, 1H), 4.80 (brs, 2H), 4.63-4.51 (m, 2H), 4.38-4.31 (m, 2H), 4.05-3.95 (m, 2H), 3.60-3.52 (m, 1H), 3.38-3.31 (m, 1H), 3.01-2.93 (m, 4H), 2.04-1.98 (m, 2H), 1.86-1.79 (m, 1H), 1.51 (s, 3H), 1.49 (s, 3H), 1.08-1.03 (m, 2H), 0.76-0.72 (m, 2H).

LC-MS (ESI): m/z=498.2 [M+H]⁺.

### Example 138

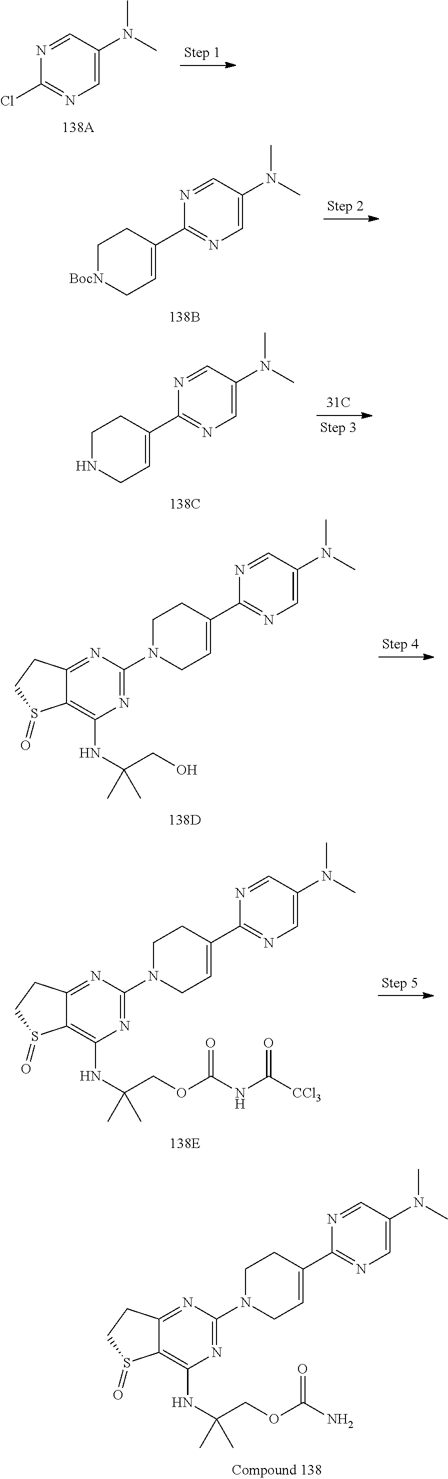

Using 138A (see document JACS-142(50)-21197 for the synthesis) as a raw material, the target compound 138 (125 mg, 81.58%) was obtained according to the operation of Example 119.

¹H NMR (400 MHz, CDCl₃) δ 8.19 (s, 2H), 7.00-6.98 (m, 1H), 5.38 (s, 1H), 4.85-4.76 (m, 2H), 4.50-4.48 (m, 2H), 4.41-4.33 (m, 2H), 4.09-4.05 (m, 2H), 3.64-3.56 (m, 1H), 3.42-3.35 (m, 1H), 3.03-2.98 (m, 8H), 2.81-2.77 (m, 2H), 1.54 (s, 3H), 1.52 (s, 3H).

LC-MS (ESI): m/z=487.2 [M+H]⁺

### Example 139

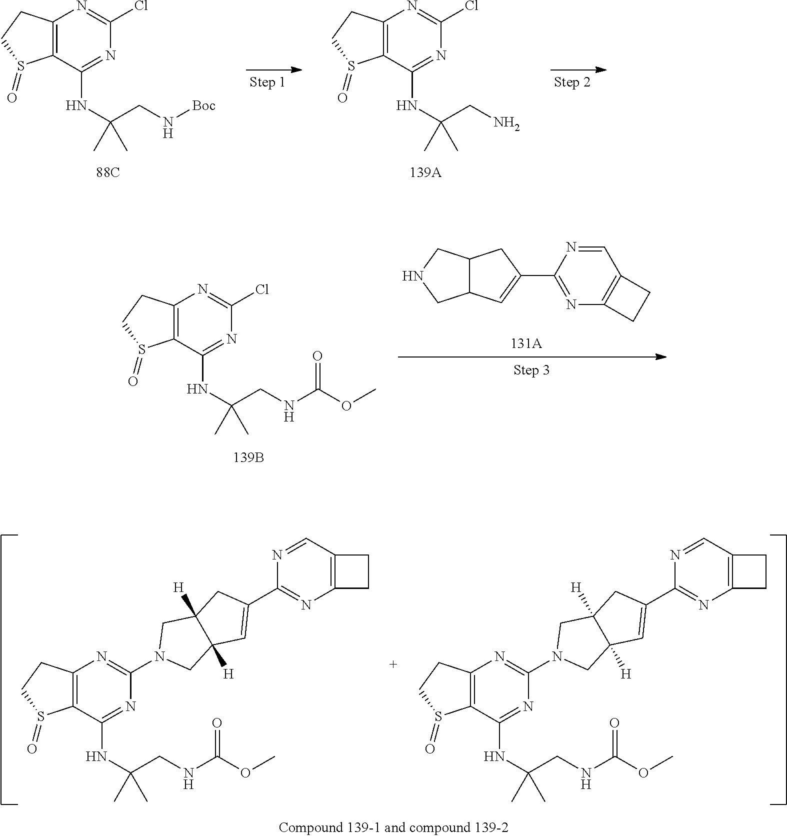

Step 1: Compound 88C (500 mg, 1.33 mmol) was dissolved in dichloromethane/trifluoroacetic acid (12 mL/4 ml) and reacted at room temperature for half an hour, and the system was then concentrated to obtain compound 139A, which was directly used for the next step.

LC-MS (ESI): m/z=275.1 [M+H]⁺

Step 2: The compound 139A trifluoroacetate compound from the previous step was dissolved in tetrahydrofuran/water (10 ml/10 ml), sodium bicarbonate (337 mg, 4.01 mmol) was added, and the mixture was placed in an ice bath and cooled. Methyl chloroformate (189 mg, 2.0 mmol) was dropwise added to the reaction system. After the reaction was complete, the system was directly spin-dried and quickly separated and purified (eluent ratio: MeOH/DCM=0-20%) to obtain compound 139B (421 mg, 95%).

LC-MS (ESI): m/z=333.1[M+H]⁺

Step 3: Using 139B as a raw material, compounds 139-1 (18.5 mg, 24%) and 139-2 (20.3 mg, 26%) were obtained by resolution according to the synthesis method in Example 131.

Resolution of compound 139: Compound 139 (60 mg) was taken for resolution, and after separation, compound 139-1 (retention time: 1.574 min, 18.5 mg, ee %=100%) and compound 139-2 (retention time: 2.485 min, 20.5 mg, ee %=100%) were obtained. Resolution conditions: instrument: Waters 150 Prep-SFC E; column: Chiralcel Cellulose-2 column; mobile phase: A for CO2; B for 0.1% NH₃·H₂O in IPA and ACN; gradient: B 70%; flow: 100 mL/min; back pressure: 100 bar; column temperature: 25° C.; wavelength: 220 nm; period: 7.0 min;

### Compound 139-1:

¹H NMR (400 MHz, CDCl₃) δ=8.24 (s, 1H), 6.81 (s, 1H), 5.51 (s, 1H), 5.24 (d, 1H), 4.10-3.95 (m, 1H), 3.94-3.76 (m, 2H), 3.70-3.54 (m, 7H), 3.50-3.42 (m, 2H), 3.41-3.28 (m, 2H), 3.23 (t, 2H), 3.20-3.14 (m, 1H), 3.10-3.06 (m, 1H), 3.04-2.93 (m, 2H), 2.88 (d, 1H), 1.46 (d, 6H).

LC-MS (ESI): m/z=510.2[M+H]⁺

### Compound 139-2:

¹H NMR (400 MHz, CDCl₃) δ=8.24 (s, 1H), 6.81 (s, 1H), 5.48 (s, 1H), 5.25 (d, 1H), 4.08-4.00 (m, 1H), 3.97-3.77 (m, 2H), 3.76-3.50 (m, 7H), 3.49-3.43 (m, 2H), 3.42-3.29 (m, 2H), 3.27-3.21 (m, 2H), 3.21-3.15 (m, 1H), 3.15-2.94 (m, 3H), 2.88 (d, 1H), 1.45 (s, 6H).

LC-MS (ESI): m/z=510.2 [M+H]⁺

### Example 140

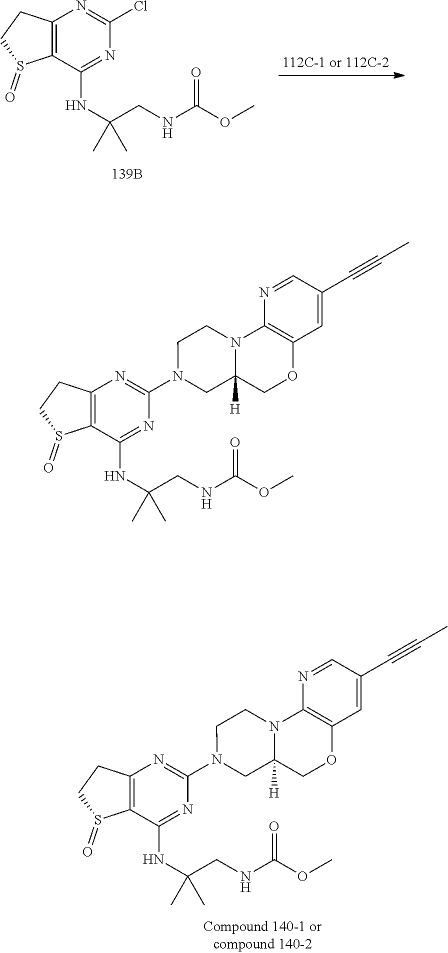 [SMILES: COC(=O)NCC(C)(C)Nc1nc(Cl)nc2c1[S@](=O)CC2 ; CC#Cc1cnc2c(c1)OC[C@@H]1CN(c3nc4c(c(NC(C)(C)CNC(=O)OC)n3)[S@](=O)CC4)CCN21 ; CC#Cc1cnc2c(c1)OC[C@H]1CN(c3nc4c(c(NC(C)(C)CNC(=O)OC)n3)[S@](=O)CC4)CCN21]

Using 139B and 112C-1 as raw materials, compound 140-1 (10 mg, 32%) was obtained according to the synthesis method in Example 112.

Using 139B and 112C-2 as raw materials, compound 140-2 (10 mg, 32%) was obtained by resolution according to the synthesis method in Example 112.

### Compound 140-1:

¹H NMR (400 MHz, CDCl₃) δ=7.86 (d, 1H), 6.96 (d, 1H), 5.65 (s, 1H), 5.29 (d, 1H), 4.84 (d, 2H), 4.61 (d, 1H), 4.35 (d, 1H), 4.03-3.99 (m, 1H), 3.69 (s, 3H), 3.68-3.48 (m, 3H), 3.47-3.31 (m, 2H), 3.13 (t, 1H), 3.08-2.95 (m, 2H), 2.92-2.74 (m, 2H), 2.03 (s, 3H), 1.47 (d, 6H).

LC-MS (ESI): m/z=526.2 [M+H]⁺

### Compound 140-2:

¹H NMR (400 MHz, CDCl₃) δ=7.86 (d, 1H), 6.96 (d, 1H), 5.65 (s, 1H), 5.28 (t, 1H), 4.84 (d, 2H), 4.61 (d, 1H), 4.35 (d, 1H), 4.03-3.99 (m, 1H), 3.69 (s, 3H), 3.62-3.56 (m, 3H), 3.45-3.33 (m, 2H), 3.19-3.09 (m, 1H), 3.06-2.97 (m, 2H), 2.90-2.74 (m, 2H), 2.03 (s, 3H), 1.47 (s, 6H).

LC-MS (ESI): m/z=526.2 [M+H]⁺

### Example 141

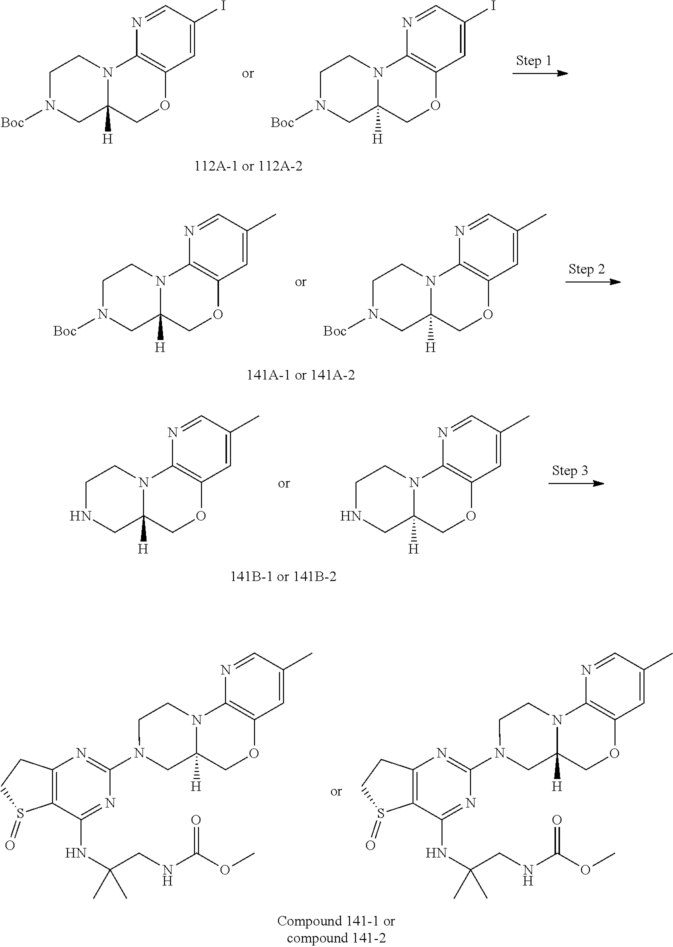

Step 1: Compound 112A-1 (400 mg, 0.96 mmol) and trimethylboroxine (1.92 mmol) were dissolved in N,N-dimethylformnamide (10 ml), and Pd(dppf)Cl₂ (73 mg, 0.01 mmol) and potassium carbonate (397 mg, 2.88 mmol) were added. After nitrogen displacement three times, the mixture was heated to 110° C. and reacted for 3 h, and the system was then concentrated and quickly separated and purified (petroleum ether/ethyl acetate=10/1) to obtain compound 141A-1 (200 mg, 68%).

LC-MS (ESI): m/z=306.2 [M+H]⁺

Using 141A-1 (100 mg, 0.33 mmol) as a raw material, compound 141-1 (76 mg, 46%) was obtained according to the synthesis method in Example 112.

According to the above method, using 112A-2 as a raw material, the target compound 141-2 (58 mg, 35%) was obtained.

### Compound 141-1:

¹H NMR (400 MHz, DMSO-d6) δ=7.57 (s, 1H), 7.43 (t, 1H), 6.87 (s, 1H), 6.72 (s, 1H), 4.79 (d, 2H), 4.42 (d, 2H), 4.04-3.92 (m, 1H), 3.58 (s, 3H), 3.48-3.38 (m, 2H), 3.34 (d, 1H), 3.27-3.19 (m, 2H), 3.09-2.92 (m, 2H), 2.89-2.84 (m, 1H), 2.76 (t, 1H), 2.67 (t, 1H), 2.12 (s, 3H), 1.40 (d, 6H).

LC-MS (ESI): m/z=502.2 [M+H]⁺

### Compound 141-2:

¹H NMR (400 MHz, DMSO-d6) δ=7.57 (s, 1H), 7.43 (t, 1H), 6.87 (s, 1H), 6.73 (s, 1H), 4.80 (d, 2H), 4.41 (d, 2H), 4.05-3.93 (m, 1H), 3.58 (s, 3H), 3.48-3.38 (m, 3H), 3.29-3.19 (m, 2H), 3.08-2.92 (m, 2H), 2.89-2.84 (m, 1H), 2.76 (t, 1H), 2.67 (t, 1H), 2.12 (s, 3H), 1.40 (d, 6H).

LC-MS (ESI): m/z=502.2 [M+H]⁺

### Example 142

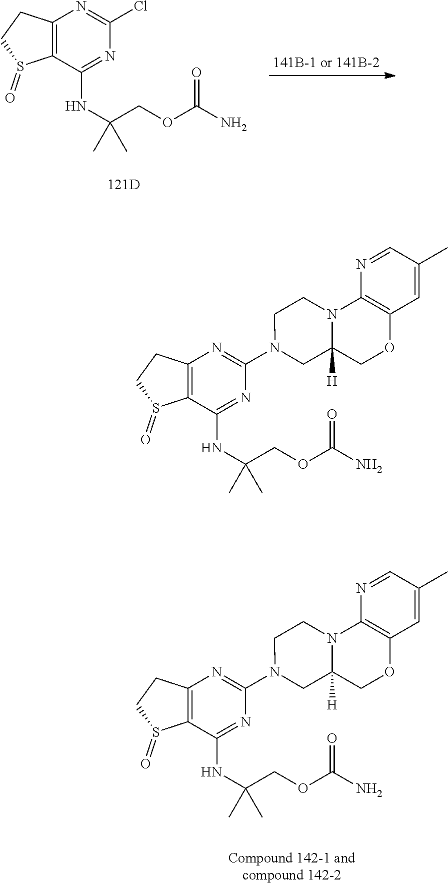 [SMILES: CC(C)(COC(N)=O)Nc1nc(Cl)nc2c1[S@](=O)CC2 ; Cc1cnc2c(c1)OC[C@@H]1CN(c3nc4c(c(NC(C)(C)COC(N)=O)n3)[S@](=O)CC4)CCN21 ; Cc1cnc2c(c1)OC[C@H]1CN(c3nc4c(c(NC(C)(C)COC(N)=O)n3)[S@](=O)CC4)CCN21]

Using 121D and 141B-1 (67 mg, 0.33 mmol) as raw materials, compound 142-1 (88 mg, 55%) was obtained according to the synthesis method in Example 121.

According to the above method, using 121D and 141B-2 (67 mg, 0.33 mmol) as raw materials, the target compound 142-2 (100 mg, 62%) was obtained.

### Compound 142-1:

¹H NMR (400 MHz, DMSO-d6) δ=7.57 (s, 1H), 6.87 (s, 1H), 6.70 (s, 1H), 6.56 (s, 2H), 4.76 (d, 2H), 4.48-4.21 (m, 4H), 4.05-3.90 (m, 1H), 3.53-3.37 (m, 1H), 3.29-3.20 (m, 2H), 3.08-2.92 (m, 2H), 2.90-2.85 (m, 1H), 2.77 (t, 1H), 2.70-2.65 (m, 1H), 2.12 (s, 3H), 1.43 (d, 6H).

LC-MS (ESI): m/z=488.2 [M+H]⁺

### Compound 142-2:

¹H NMR (400 MHz, DMSO-d6) δ=7.57 (s, 1H), 6.87 (d, 1H), 6.71 (s, 1H), 6.56 (s, 2H), 4.77 (d, 2H), 4.47-4.20 (m, 4H), 4.03-3.92 (m, 1H), 3.50-3.36 (m, 1H), 3.28-3.23 (m, 2H), 3.10-2.92 (m, 2H), 2.92-2.83 (m, 1H), 2.77 (t, 1H), 2.67 (t, 1H), 2.12 (s, 3H), 1.43 (d, 6H).

LC-MS (ESI): m/z=488.2 [M+H]⁺

### Example 143

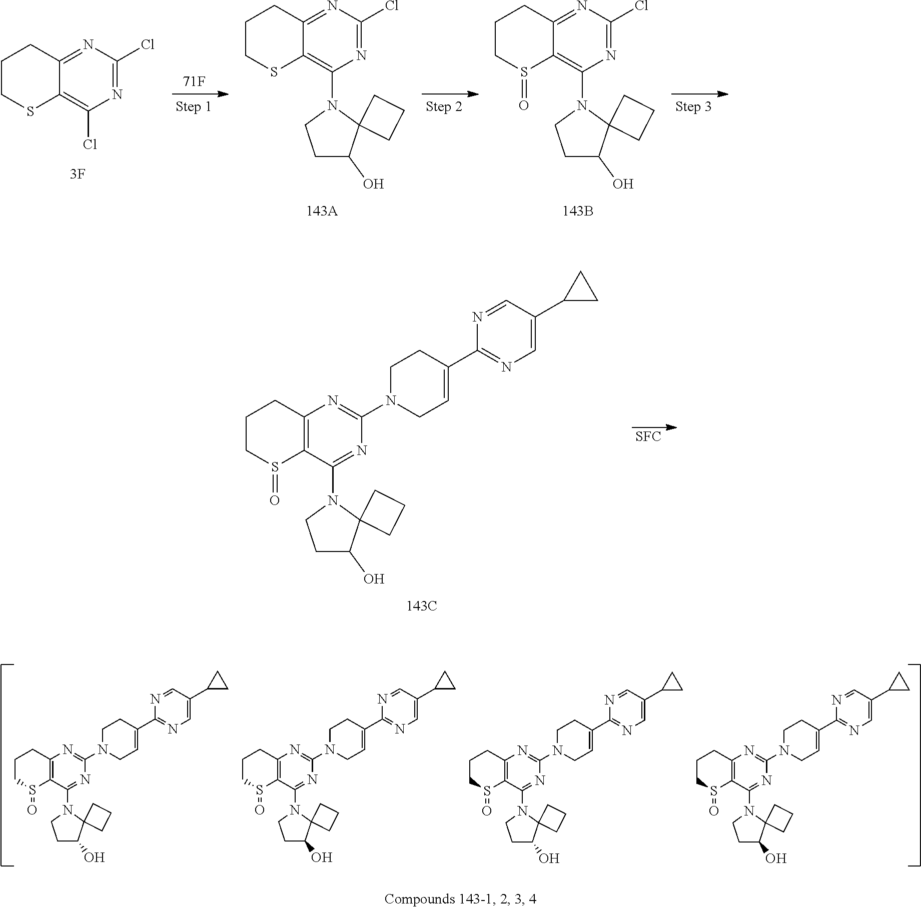

Using 3F and compound 71F as raw materials, the target compound, racemate 143C, was synthesized according to the operation of Example 114, and the racemate was resolved by SFC to obtain compound 143-1 (25 mg), compound 143-2 (13 mg), compound 143-3 (26 mg), and compound 143-4 (12 mg).

SFC preparation method: instrument: Waters 150 Prep-SFC F, column: Chiralcel Cellulose-2 Column; mobile phase: A: CO₂, B: 0.1% NH₃·H₂O in IPA and ACN; gradient: 60% B gradient elution; flow rate: 100 mL/min, column temperature: 25° C.; wavelength: 220 nm; cycle time: 8.6 min; sample preparation: sample concentration 10 mg/ml, acetonitrile solution injection: 1.5 ml each time. After separation, the fraction was dried by a rotary evaporator at a bath temperature of 35° C. to obtain compound 143-1 (25.2 mg), compound 143-2 (13.7 mg), compound 143-3 (26.1 mg), and compound 143-4 (12.0 mg).

Compound 143-1, retention time: 1.638 min; ¹H NMR (400 MHz, CDCl₃) δ 8.54 (s, 2H), 7.21 (s, 1H), 5.27 (d, 1H), 4.49 (s, 2H), 3.97-4.18 (m, 5H), 3.45-3.52 (m, 1H), 3.28 (s, 2H), 3.04-3.11 (m, 1H), 2.83-2.91 (m, 1H), 2.65-2.75 (m, 4H), 2.19-2.28 (m, 1H), 1.69-1.95 (m, 6H), 1.55-1.64 (m, 1H), 1.01-1.06 (m, 2H), 0.81-0.85 (m, 2H).

LC-MS (ESI): m/z=493.2 [M+H]⁺

Compound 143-2, retention time: 1.745 min; ¹H NMR (400 MHz, CDCl₃) δ 8.54 (s, 2H), 7.21 (s, 1H), 5.27 (d, 1H), 4.49 (s, 2H), 3.97-4.18 (m, 5H), 3.45-3.52 (m, 1H), 3.28 (s, 2H), 3.04-3.11 (m, 1H), 2.83-2.91 (m, 1H), 2.65-2.75 (m, 4H), 2.19-2.28 (m, 1H), 1.69-1.95 (m, 6H), 1.55-1.64 (m, 1H), 1.01-1.06 (m, 2H), 0.81-0.85 (m, 2H).

LC-MS (ESI): m/z=493.2 [M+H]⁺

Compound 143-3, retention time: 1.794 min; ¹H NMR (400 MHz, CDCl₃) δ 8.54 (s, 2H), 7.21 (s, 1H), 5.27 (d, 1H), 4.49 (s, 2H), 3.97-4.18 (m, 5H), 3.45-3.52 (m, 1H), 3.28 (s, 2H), 3.04-3.11 (m, 1H), 2.83-2.91 (m, 1H), 2.65-2.75 (m, 4H), 2.19-2.28 (m, 1H), 1.69-1.95 (m, 6H), 1.55-1.64 (m, 1H), 1.01-1.06 (m, 2H), 0.81-0.85 (m, 2H).

LC-MS (ESI): m/z=493.2 [M+H]⁺

Compound 143-4, retention time: 2.558 min; ¹H NMR (400 MHz, CDCl₃) δ 8.54 (s, 2H), 7.21 (s, 1H), 5.27 (d, 1H), 4.49 (s, 2H), 3.97-4.18 (m, 5H), 3.45-3.52 (m, 1H), 3.28 (s, 2H), 3.04-3.11 (m, 1H), 2.83-2.91 (m, 1H), 2.65-2.75 (m, 4H), 2.19-2.28 (m, 1H), 1.69-1.95 (m, 6H), 1.55-1.64 (m, 1H), 1.01-1.06 (m, 2H), 0.81-0.85 (m, 2H).

LC-MS (ESI): m/z=493.2 [M+H]⁺

### Example 144

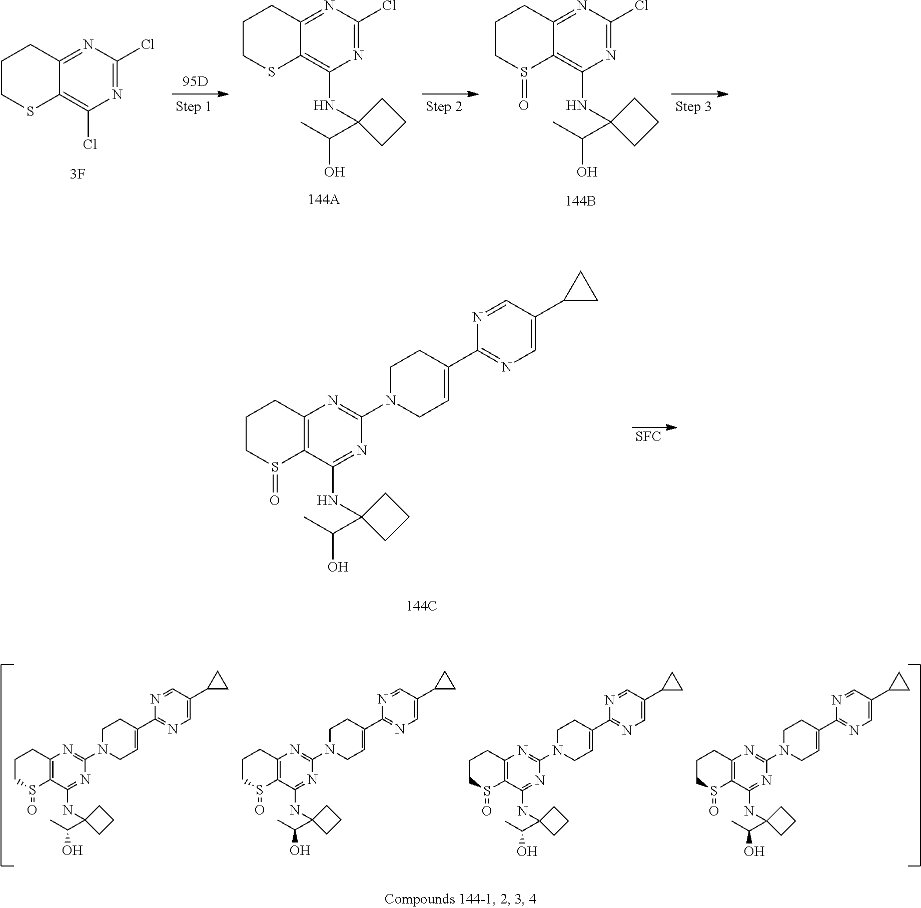

Using 3F and compound 95D as raw materials, the target compound, racemate 144C, was obtained according to the operation of Example 95, and the racemate was resolved by SFC to obtain compound 144-1 (4.8 mg), compound 144-2 (5.2 mg), compound 144-3 (4.6 mg), and compound 144-4 (2.6 mg).

SFC preparation method: instrument: Waters 150 Prep-SFC F, column: Chiralcel Cellulose-2 Column; mobile phase: A: CO2, B: 0.1% NH₃·H₂O in IPA and ACN; gradient: 50% B gradient elution; flow rate: 100 mL/min, column temperature: 25° C.; wavelength: 220 nm; cycle time: 8.6 min; sample preparation: sample concentration 10 mg/ml, acetonitrile solution injection: 1.5 ml each time. After separation, the fraction was dried by a rotary evaporator at a bath temperature of 35° C. to obtain compound 144-1 (4.8 mg), compound 144-2 (5.2 mg), compound 144-3 (4.6 mg), and compound 144-4 (2.6 mg).

Compound 144-1, retention time: 1.328 min; ¹H NMR (400 MHz, CDCl₃) δ 8.54 (s, 2H), 7.18 (s, 1H), 6.49 (d, 1H), 4.90 (s, 1H), 4.41 (s, 2H), 3.93-4.04 (m, 3H), 3.27 (s, 2H), 3.04-3.10 (m, 1H), 2.87-2.94 (m, 1H), 2.57-2.71 (m, 4H), 2.11-2.17 (m, 1H), 1.89-2.00 (m, 2H), 1.68-1.79 (m, 2H), 1.48-1.58 (m, 2H), 1.21 (s, 3H), 1.00-1.05 (m, 2H), 0.81-0.85 (m, 2H).

LC-MS (ESI): m/z=481.2 [M+H]⁺

Compound 144-2, retention time: 1.425 min; ¹H NMR (400 MHz, CDCl₃) δ 8.54 (s, 2H), 7.18 (s, 1H), 6.49 (d, 1H), 4.90 (s, 1H), 4.41 (s, 2H), 3.93-4.04 (m, 3H), 3.27 (s, 2H), 3.04-3.10 (m, 1H), 2.87-2.94 (m, 1H), 2.57-2.71 (m, 4H), 2.11-2.17 (m, 1H), 1.89-2.00 (m, 2H), 1.68-1.79 (m, 2H), 1.48-1.58 (m, 2H), 1.21 (s, 3H), 1.00-1.05 (m, 2H), 0.81-0.85 (m, 2H).

LC-MS (ESI): m/z=481.2 [M+H]⁺

Compound 144-3, retention time: 2.012 min; ¹H NMR (400 MHz, CDCl₃) δ 8.54 (s, 2H), 7.18 (s, 1H), 6.49 (d, 1H), 4.90 (s, 1H), 4.41 (s, 2H), 3.93-4.04 (m, 3H), 3.27 (s, 2H), 3.04-3.10 (m, 1H), 2.87-2.94 (m, 1H), 2.57-2.71 (m, 4H), 2.11-2.17 (m, 1H), 1.89-2.00 (m, 2H), 1.68-1.79 (m, 2H), 1.48-1.58 (m, 2H), 1.21 (s, 3H), 1.00-1.05 (m, 2H), 0.81-0.85 (m, 2H).

LC-MS (ESI): m/z=481.2 [M+H]⁺

Compound 144-4, retention time: 2.302 min; ¹H NMR (400 MHz, CDCl₃) δ 8.54 (s, 2H), 7.18 (s, 1H), 6.49 (d, 1H), 4.90 (s, 1H), 4.41 (s, 2H), 3.93-4.04 (m, 3H), 3.27 (s, 2H), 3.04-3.10 (m, 1H), 2.87-2.94 (m, 1H), 2.57-2.71 (m, 4H), 2.11-2.17 (m, 1H), 1.89-2.00 (m, 2H), 1.68-1.79 (m, 2H), 1.48-1.58 (m, 2H), 1.21 (s, 3H), 1.00-1.05 (m, 2H), 0.81-0.85 (m, 2H).

LC-MS (ESI): m/z=481.2 [M+H]⁺

### Example 145

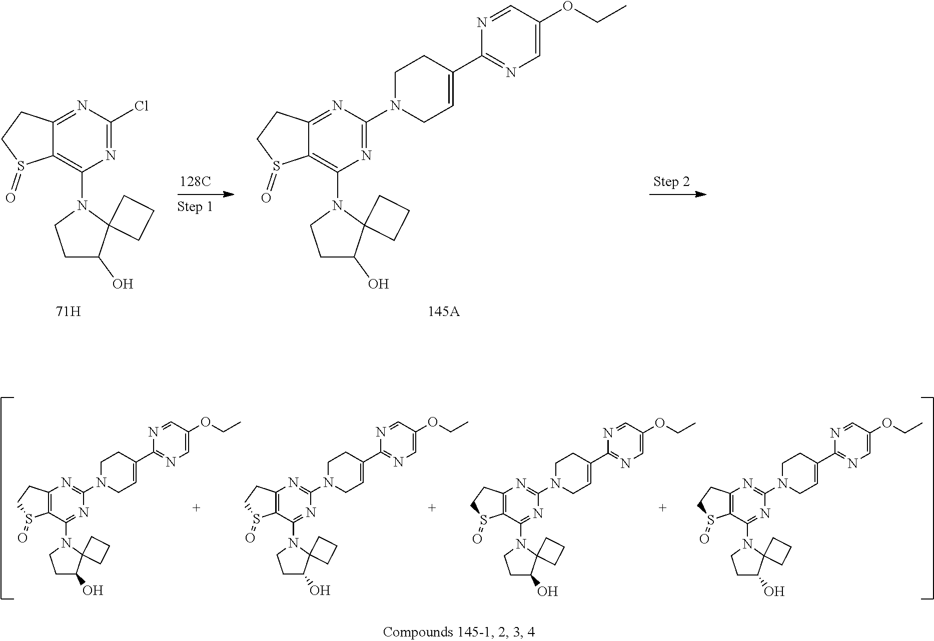

Step 1: Under nitrogen protection, 71H (0.78 g, 2.49 mmol), intermediate 128C (0.90 g, 3.73 mmol), and N,N-diisopropylethylamine (0.96 g, 7.46 mmol) were successively dissolved in 1,4-dioxane (20 mL), heated to 85° C., and stirred for 14 hours. The reaction liquid was cooled to room temperature, then diluted by adding water (80 mL), and extracted three times with dichloromethane (50 mL). The organic phases were combined and dried over anhydrous sodium sulfate. After filtration, the filtrate was concentrated and then purified and separated by silica gel column chromatography (dichloromethane/methanol (v/v)=90/10) to obtain racemic compound 145A (1.20 g, yield: 96.07%).

LC-MS (ESI): m/z=483.6 [M+H]⁺

Step 2: Compound 145A (1.20 g, 2.49 mmol) was subjected to reverse phase preparation to obtain P1 (500 mg) and P2 (450 mg), which were then respectively subjected to chiral resolution to obtain compound 145-1 (113.2 mg), compound 145-2 (91.6 mg), compound 145-3 (163.8 mg), and compound 145-4 (176.8 mg).

HPLC analysis method: instrument: SHIMADZU LC-2020AD, column: C18 Column; mobile phase: A: 0.1% TFA in H2O, B: ACN; gradient: 20-80% B in A; flow rate: 1.2 mL/min, column temperature: 45° C., wavelength: 210 nm & 254 nm.

HPLC preparation method: instrument: SHIMADZU LC-20AP, column: XB—SiOH Column; mobile phase: A: hexane, B: EtOH; gradient: 15-55% B gradient elution; flow rate: 80 mL/min, column temperature: 25° C.; wavelength: 220 nm; cycle time: 5.0 min, sample preparation: sample concentration 50 mg/ml, acetonitrile solution injection: 8.0 ml each time.

After separation, the fraction was dried by a rotary evaporator at a bath temperature of 35° C. to obtain compound P1 and compound P2.

SFC analysis method: instrument: SHIMADZU LC-2020AD, column: Chiralcel IH Column; mobile phase: A: Hexane, B: 0.1% IPAm IPA and ACN; gradient: 30% B in A; flow rate: 1.0 mL/min, column temperature: 35° C., wavelength: 254 nm.

SFC preparation method: instrument: SHIMADZU LC-20AP, column: Chiralcel IH Column; mobile phase: A: hexane, B: 0.1% IPAm IPA and ACN; gradient: 67% B gradient elution; flow rate: 60 mL/min, column temperature: 25° C.; wavelength: 220 nm & 254 nm; cycle time: 6.0 min; sample preparation: sample concentration 10 mg/ml, acetonitrile solution injection: 4.0 ml each time.

After separation, the fraction was dried by a rotary evaporator at a bath temperature of 35° C. to obtain compound 145-1 (113.2 mg), compound 145-2 (91.6 mg), compound 145-3 (163.8 mg), and compound 145-4 (176.8 mg).

Compound 145-1: retention time: 2.516 min; ¹H NMR (400 MHz, CDCl₃) δ 8.36 (s, 2H), 7.14-7.12 (m, 1H), 4.65-4.55 (m, 2H), 4.21-4.12 (m, 6H), 3.76-3.62 (m, 3H), 3.48-3.41 (m, 1H), 3.18-3.07 (m, 3H), 2.82 (s, 2H), 2.27-2.22 (m, 1H), 2.08-2.07 (m, 2H), 1.99-1.91 (m, 1H), 1.85-1.80 (m, 2H), 1.72-1.67 (m, 1H), 1.48-1.45 (m, 3H).

LC-MS (ESI): m/z=483.0 [M+H]⁺

Compound 145-2: retention time: 4.322 min; ¹H NMR (400 MHz, CDCl₃) δ 8.36 (s, 2H), 7.14-7.12 (m, 1H), 4.65-4.55 (m, 2H), 4.21-4.11 (m, 6H), 3.76-3.62 (m, 3H), 3.48-3.41 (m, 1H), 3.18-3.07 (m, 3H), 2.82 (s, 2H), 2.27-2.22 (m, 1H), 2.08-2.07 (m, 2H), 1.99-1.91 (m, 1H), 1.85-1.80 (m, 2H), 1.72-1.67 (m, 1H), 1.48-1.45 (m, 3H).

LC-MS (ESI): m/z=483.0 [M+H]⁺

Compound 145-3: retention time: 2.490 min; ¹H NMR (400 MHz, CDCl₃) δ 8.36 (s, 2H), 7.14-7.12 (m, 1H), 4.65-4.55 (m, 2H), 4.21-4.12 (m, 6H), 3.76-3.62 (m, 3H), 3.48-3.41 (m, 1H), 3.18-3.07 (m, 3H), 2.82 (s, 2H), 2.27-2.22 (m, 1H), 2.08-2.06 (m, 2H), 1.99-1.91 (m, 1H), 1.85-1.80 (m, 2H), 1.72-1.67 (m, 1H), 1.48-1.45 (m, 3H).

LC-MS (ESI): m/z=483.0 [M+H]⁺

Compound 145-4: retention time: 3.794 min; ¹H NMR (400 MHz, CDCl₃) δ 8.36 (s, 2H), 7.14-7.12 (m, 1H), 4.65-4.55 (m, 2H), 4.21-4.12 (m, 6H), 3.76-3.62 (m, 3H), 3.48-3.41 (m, 1H), 3.18-3.07 (m, 3H), 2.82 (s, 2H), 2.27-2.21 (m, 1H), 2.08-2.07 (m, 2H), 1.99-1.91 (m, 1H), 1.85-1.80 (m, 2H), 1.72-1.67 (m, 1H), 1.48-1.45 (m, 3H).

LC-MS (ESI): m/z=483.0 [M+H]⁺

### Example 146

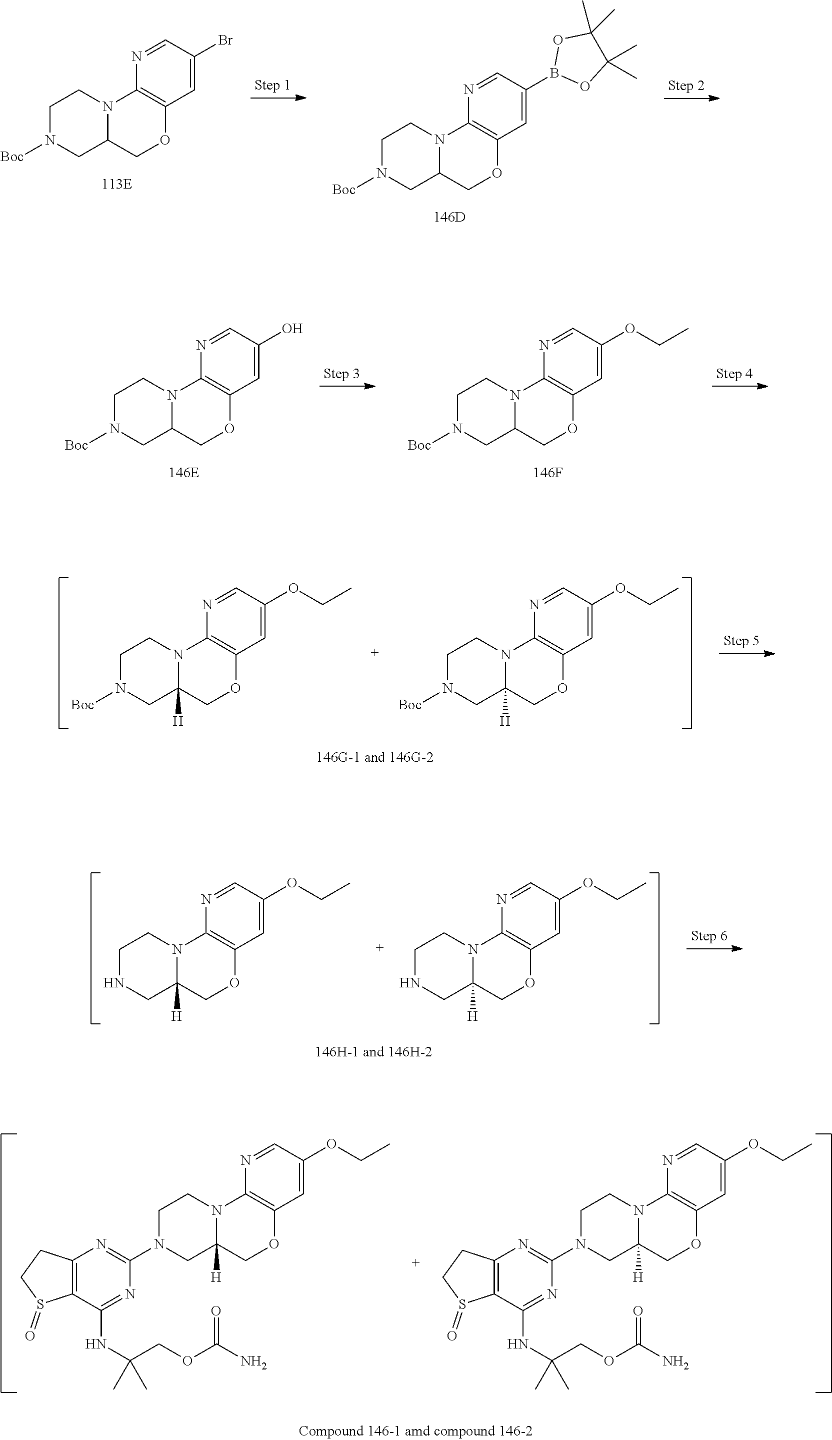

Step 1: Compound 113E (5 g, 13.55 mmol) was dissolved in dry toluene (50 ml), and pinacol diboronate (0.88 g, 27.1 mmol), [1,1′-bis(diphenylphosphine)ferrocene]palladium dichloride dichloromethane complex (1.98 g, 2.71 mmol), and sodium acetate (2.22 g, 27.1 mmol) were added. After nitrogen displacement three times, the mixture was heated to 100° C. and further reacted under stirring for 4 h. After the reaction was complete, the system was cooled to room temperature, and the reaction was quenched by adding water (50 mL). The system was extracted with ethyl acetate (50 mL×3), and the organic phases were combined, washed with a saturated sodium chloride aqueous solution, and dried over anhydrous sodium sulfate. After filtration, the reaction liquid was concentrated under reduced pressure and then separated and purified by silica gel column chromatography (petroleum ether/ethyl acetate (v/v)=100/0-70/30) to obtain the target compound 146D (4.8 g, 85%).

LC-MS (ESI): m/z=418.2 [M+H]⁺%

Step 2: Compound 146D (1.2 g, 2.87 mmol) was dissolved in acetone (20 ml), and hydrogen peroxide (0.24 ml, 8.61 mmol) and sodium bicarbonate (1.69 g, 20.09 mmol) were added and stirred at room temperature overnight. After the reaction was complete as detected by TLC, the reaction system was diluted by adding water and dichloromethane. The aqueous phase was extracted with dichloromethane. The organic phases were collected, combined, dried over anhydrous sodium sulfate, concentrated, and quickly separated and purified (petroleum ether/ethyl acetate=5/1) to obtain compound 146E (0.8 g, 91%).

LC-MS (ESI): m/z=308.2 [M+H]⁺.

Step 3: Compound 146E (0.8 g, 2.61 mmol) was dissolved in acetone (10 ml), and cesium carbonate (2.55 g, 7.83 mmol) and iodoethane (0.82 g, 5.22 mmol) were added and stirred at room temperature for 3 h. After the reaction was complete, the reaction was quenched by adding water and extracted with ethyl acetate. The organic phases were collected, combined, dried over anhydrous sodium sulfate, concentrated, and quickly separated and purified (petroleum ether/ethyl acetate=10/1) to obtain compound 146F (0.8 g, 92%).

LC-MS (ESI): m/z=336.2 [M+H]⁺.

Step 4: Compound 146F (0.8 g) was separated by SFC to obtain two isomers, i.e., compound 146G-1 (373 mg, retention time 5.86 min) and compound 146G-2 (340 mg, retention time 7.27 min).

Separation conditions of preparative chromatography: 1. instrument: Waters 150 AP-SFC; 2. chromatographic column: AD; 3. mobile phase system: A for CO₂; B for MeOH; 4. gradient: B 28%; and 5. flow rate: 40 mL/min

Using 146G-1 (100 mg, 0.3 mmol) as a raw material, compound 146-1 (105 mg, 68%) was obtained according to the synthesis method in Example 112.

According to the above method, using 146G-2 as a raw material, the target compound 146-2 (100 mg, 64%) was obtained.

### Compound 146-1:

¹H NMR (400 MHz, DMSO-d6) δ=7.50 (d, 1H), 6.78 (d, 1H), 6.70 (s, 1H), 6.56 (s, 2H), 4.77 (t, 2H), 4.46-4.21 (m, 4H), 4.05-3.92 (m, 3H), 3.49-3.36 (m, 1H), 3.28-3.16 (m, 2H), 3.10-2.92 (m, 2H), 2.92-2.82 (m, 1H), 2.76 (t, 1H), 2.67-2.60 (m, 1H), 1.43 (d, 6H), 1.28 (t, 3H).

LC-MS (ESI): m/z=518.2 [M+H]⁺

### Compound 146-2:

¹H NMR (400 MHz, DMSO-d6) δ=7.50 (d, 1H), 6.78 (d, 1H), 6.71 (s, 1H), 6.56 (s, 2H), 4.77 (d, 2H), 4.49-4.19 (m, 4H), 4.05-3.89 (m, 3H), 3.51-3.38 (m, 1H), 3.19 (t, 2H), 3.11-2.92 (m, 2H), 2.91-2.86 (m, 1H), 2.75 (t, 1H), 2.63 (t, 1H), 1.43 (d, 6H), 1.28 (t, 3H).

LC-MS (ESI): m/z=518.2 [M+H]⁺

### Example 147

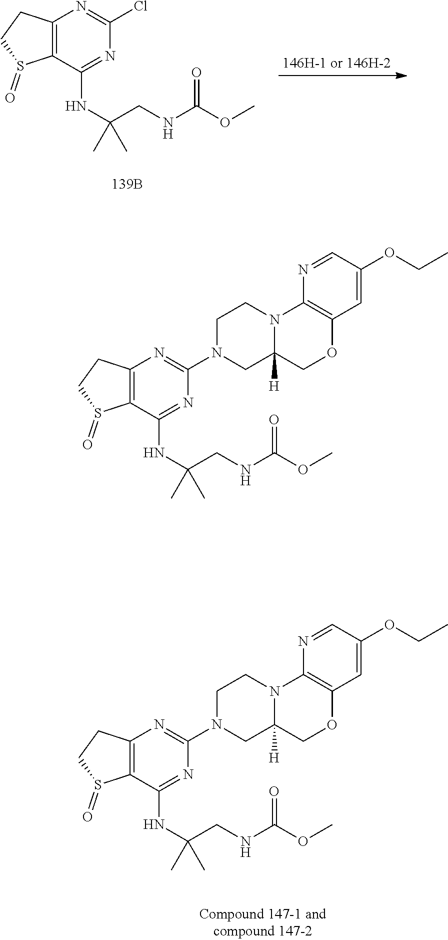 [SMILES: COC(=O)NCC(C)(C)Nc1nc(Cl)nc2c1[S@](=O)CC2 ; CCOc1cnc2c(c1)OC[C@@H]1CN(c3nc4c(c(NC(C)(C)CNC(=O)OC)n3)[S@](=O)CC4)CCN21 ; CCOc1cnc2c(c1)OC[C@H]1CN(c3nc4c(c(NC(C)(C)CNC(=O)OC)n3)[S@](=O)CC4)CCN21]

Step 1: Using 139B (100 mg, 0.3 mmol) and 146H-1 as raw materials, compound 147-1 (100 mg, 63%) was obtained according to the synthesis method in Example 112.

Using 139B (100 mg, 0.3 mmol) and 146H-2 as raw materials, the target compound 147-2 (101 mg, 63%) was obtained according to the synthesis method in Example 112.

### Compound 147-1:

¹H NMR (400 MHz, DMSO-d6) δ=7.50 (d, 1H), 7.43 (t, 1H), 6.78 (d, 1H), 6.72 (s, 1H), 4.80 (d, 2H), 4.44 (d, 1H), 4.35 (d, 1H), 4.06-3.92 (m, 3H), 3.57 (s, 3H), 3.48-3.38 (m, 3H), 3.26-3.21 (m, 2H), 3.04 (t, 1H), 3.00-2.91 (m, 1H), 2.91-2.82 (m, 1H), 2.75 (t, 1H), 2.67-2.61 (m, 1H), 1.47-1.34 (m, 6H), 1.28 (t, 3H).

LC-MS (ESI): m/z=532.2 [M+H]⁺

### Compound 147-2:

¹H NMR (400 MHz, DMSO-d6) δ=7.50 (d, 1H), 7.43 (t, 1H), 6.78 (d, 1H), 6.72 (s, 1H), 4.80 (d, 2H), 4.45 (d, 1H), 4.34 (d, 1H), 4.06-3.90 (m, 3H), 3.57 (s, 3H), 3.44-3.38 (m, 3H), 3.27-3.15 (m, 2H), 3.12-2.92 (m, 2H), 2.91-2.82 (m, 1H), 2.75 (t, 1H), 2.64 (t, 1H), 1.40 (d, 6H), 1.28 (t, 3H).

LC-MS (ESI): m/z=532.2 [M+H]⁺

### Example 148

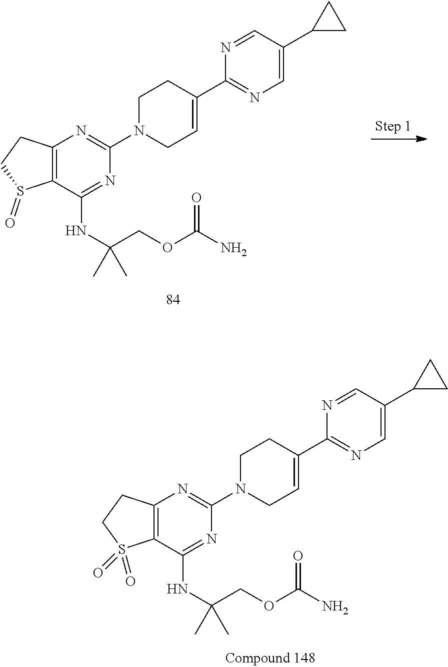 [SMILES: CC(C)(COC(N)=O)Nc1nc(N2CC=C(c3ncc(C4CC4)cn3)CC2)nc2c1[S@](=O)CC2 ; CC(C)(COC(N)=O)Nc1nc(N2CC=C(c3ncc(C4CC4)cn3)CC2)nc2c1S(=O)(=O)CC2]

Step 1: Compound 84 (500 mg, 1.03 mmol) was dissolved in dichloromethane (15 mL), and m-chloroperoxybenzoic acid (210 m g, 1.24 mmol) was added and reacted at room temperature for 16 h. The system was then quenched by adding a saturated sodium thiosulfate solution. The aqueous phase was extracted with dichloromethane (20 mL×3), and the organic phases were combined and dried over anhydrous sodium sulfate. After filtration, the filtrate was concentrated, and the crude product was purified by column chromatography (dichloromethane:methanol=10:1 (v/v)) to obtain the target compound 148 (190 mg, yield: 47%).

LC-MS (ESI): m/z=500.1 [M+1]⁺.

¹H NMR (400 MHz, DMSO-d6) δ 8.55 (s, 2H), 7.19 (t, 1H), 6.54 (s, 2H), 5.46 (s, 1H), 4.48 (s, 2H), 4.27 (s, 2H), 4.01 (t, 2H), 3.49 (t, 2H), 3.09 (t, 2H), 2.68 (d, 2H), 2.01-1.83 (m, 1H), 1.47 (s, 6H), 1.11-0.97 (m, 2H), 0.92-0.76 (m, 2H).

### Biological Test:

### 1. Effect of Compounds on PDE4B2 Activity

The effect of compounds on PDE4B2 activity was detected using a fluorescence polarization kit (BPS Bioscience, Catalog #60343). According to the instructions of the kit, FAM-Cyclic-3′,5′-AMP at a final concentration of 0.1 M, 1 ng/well of PDE4B2 (for a negative control, a PDE buffer was added), and gradient-diluted compounds (for a positive control well, a PDE buffer containing 10% of DMSO was added) were added to each well, fully mixed, and then reacted at room temperature for 1 h. The Binding Agent was diluted with the Binding Agent Diluent (cAMP) at a ratio of 1:100 for later use, the Binding Agent dilution was taken and added to the test plate at 50 μl/well and incubated at room temperature with slow shaking for 20 minutes. After the incubation was complete, Envision was used for FP detection, with Excitation at 480 nm and Emission at 535 nm. FP was usually represented by mP value.

$$mP = ( \frac{{I}_{II} - G ( {I}_{⊥} )}{{I}_{II} + G ( {I}_{⊥} )} ) \times 1000$$

I_{∥} (S535): fluorescence intensity in parallel direction
I_{⊥} (P535): fluorescence intensity in perpendicular direction
G: G factor=1

### Calculation of Inhibition Rate (% Inhibition):

$$\% Inhibition = [ 1 - ( {mP}_{( sample )} - {mP}_{( negative control )} ) / \\ ( {mP}_{( positive control )} - {mP}_{( negative control )} ) ] \times 100 \%$$

mP₍ₛₐₘₚₗₑ₎: mP of the test compound in a reaction well
mP_{(negative control)}: mP in the negative control well
mP_{(positive control)}: mP in the positive control well

According to the calculated inhibition rate at each concentration, the IC₅₀ value of each compound was calculated by GraphPad Prism 8 software.

The compounds of the present disclosure had an IC₅₀ value<300 nM against PDE4B2; preferably, some compounds had an IC₅₀ value<100 nM; more preferably, some compounds had an IC₅₀ value<50 nM; and further preferably, some compounds had an IC₅₀ value<10 nM.

The compounds of the present disclosure had an IC₅₀ value of less than 300 nM against PDE4B2, preferably less than 100 nM, more preferably less than 50 nM, and further preferably less than 10 nM. The IC₅₀ values of some specific compounds are as shown in Table 1 below, wherein A<10 nM, 10 nM≤B<50 nM, and 50 nM≤C<100 nM.

TABLE 1
PDE4B2 activity

| | PDE4B2 IC₅₀ |
|---|---|
| Compound | (nM) |
| Compound 3 | B |
| Compound 12-2 | A |
| Compound 13-2 | A |
| Compound 14-1 | B |
| Compound 15-2 | A |
| Compound 16-2 | B |
| Compound 17-2 | A |
| Compound 19-1 | A |
| Compound 20 | A |
| Compound 23 | A |
| Compound 24-2 | A |
| Compound 26 | B |
| Compound 30 | A |
| Compound 31 | B |
| Compound 32-2 | A |
| Compound 33-2 | B |
| Compound 34 | A |
| Compound 35 | A |
| Compound 36-1 | A |
| Compound 36-2 | B |
| Compound 39 | A |
| Compound 41 | A |
| Compound 43 | A |
| Compound 44 | A |
| Compound 45-1 | A |
| Compound 45-2 | A |
| Compound 46-1 | A |
| Compound 46-2 | A |
| Compound 47 | A |
| Compound 48 | A |
| Compound 49 | A |
| Compound 50 | A |
| Compound 51 | A |
| Compound 52 | A |
| Compound 54 | C |
| Compound 55 | C |
| Compound 56 | A |
| Compound 57 | A |
| Compound 58 | A |
| Compound 60-2 | B |
| Compound 63 | A |
| Compound 64 | A |
| Compound 65 | A |
| Compound 66 | A |
| Compound 69 | A |
| Compound 70 | A |
| Compound 71-3 | B |
| Compound 71-4 | A |
| Compound 72-3 | A |
| Compound 72-4 | A |
| Compound 73-1 | A |
| Compound 73-2 | A |
| Compound 74-2 | A |
| Compound 75-1 | B |
| Compound 76-1 | A |
| Compound 77-1 | A |
| Compound 77-2 | B |
| Compound 77-3 | A |
| Compound 78 | B |
| Compound 79 | A |
| Compound 80-2 | B |
| Compound 81-1 | A |
| Compound 82-2 | A |
| Compound 83 | A |
| Compound 84 | A |
| Compound 85-1 | A |
| Compound 88 | A |
| Compound 89-2 | B |
| Compound 90 | B |
| Compound 91-1 | A |
| Compound 92-1 | A |
| Compound 92-2 | A |
| Compound 93-1 | A |
| Compound 94-1 | A |
| Compound 94-2 | A |
| Compound 95-1 | A |
| Compound 95-2 | A |
| Compound 95-3 | A |
| Compound 95-4 | A |
| Compound 96-1 | A |
| Compound 96-2 | A |
| Compound 96-3 | A |
| Compound 96-4 | B |
| Compound 97 | A |
| Compound 98 | A |
| Compound 100 | A |
| Compound 101 | A |
| Compound 102-1 | B |
| Compound 102-2 | A |
| Compound 103-2 | A |
| Compound 104 | B |
| Compound 105 | A |
| Compound 106 | B |
| Compound 107 | B |
| Compound 108 | B |
| Compound 109-2 | B |
| Compound 109-4 | A |
| Compound 110-1 | A |
| Compound 110-2 | A |
| Compound 111-2 | A |
| Compound 111-4 | A |
| Compound 112-1 | A |
| Compound 112-2 | B |
| Compound 113-2 | B |
| Compound 114-1 | B |
| Compound 115-1 | B |
| Compound 116 | B |
| Compound 117 | B |
| Compound 118 | B |
| Compound 119 | B |
| Compound 121-1 | A |
| Compound 121-2 | B |
| Compound 122 | B |
| Compound 123-1 | A |
| Compound 123-2 | A |
| Compound 124 | B |
| Compound 125 | B |
| Compound 126 | A |
| Compound 127-1 | B |
| Compound 127-2 | B |
| Compound 128 | A |
| Compound 129 | A |
| Compound 130 | B |
| Compound 131-1 | B |
| Compound 131-2 | B |
| Compound 132 | B |
| Compound 133-1 | A |
| Compound 133-2 | A |
| Compound 134 | A |
| Compound 135 | A |
| Compound 136 | A |
| Compound 137 | B |
| Compound 138 | B |
| Compound 139-1 | A |
| Compound 139-2 | B |
| Compound 140-1 | B |
| Compound 140-2 | B |
| Compound 141-1 | B |
| Compound 141-2 | B |
| Compound 142-1 | A |
| Compound 142-2 | B |
| Compound 143-3 | A |
| Compound 143-4 | A |
| Compound 144-1 | B |
| Compound 144-2 | A |
| Compound 144-4 | A |
| Compound 145-1 | A |
| Compound 145-4 | A |
| Compound 146-1 | B |
| Compound 146-2 | B |
| Compound 147-1 | B |
| Compound 147-2 | B |

### 2. Effect of Compounds on PDE4D3 Activity

The effect of compounds on PDE4D3 activity was detected using a fluorescence polarization kit (BPS Bioscience, Catalog #60346). According to the instructions of the kit, FAM-Cyclic-3′,5′-AMP at a final concentration of 0.1 M, 0.05 ng/well of PDE4D3 (for a negative control, a PDE buffer was added), and gradient-diluted compounds (for a positive control well, a PDE buffer containing 10% of DMSO was added) were added to each well, fully mixed, and then reacted at room temperature for 1 h. The Binding Agent was diluted with the Binding Agent Diluent (cAMP) at a ratio of 1:100 for later use, the Binding Agent dilution was taken and added to the test plate at 100 l/well and incubated at room temperature with slow shaking for 1 h. After the incubation was complete, Envision was used for FP detection, with Excitation at 480 nm and Emission at 535 nm. FP was usually represented by mP value.

$$mP = ( \frac{{I}_{II} - G ( {I}_{⊥} )}{{I}_{II} + G ( {I}_{⊥} )} ) \times 1000$$

I_{∥} (S535): fluorescence intensity in parallel direction
I_{⊥} (P535): fluorescence intensity in perpendicular direction
G: G factor=1

### Calculation of Inhibition Rate (% Inhibition):

$$\% Inhibition = [ 1 - ( {mP}_{( sample )} - {mP}_{( negative control )} ) / \\ ( {mP}_{( positive control )} - {mP}_{( negative control )} ) ] \times 100 \%$$

mP₍ₛₐₘₚₗₑ₎: mP of the test compound in a reaction well
mP_{(negative control)}: mP in the negative control well
mP_{(positive control)}: mP in the positive control well

According to the calculated inhibition rate at each concentration, the IC₅₀ value of each compound was calculated by GraphPad Prism 8 software.

### 3. Effect of Compounds on PDE4B1 Activity

The effect of compounds on PDE4B1 activity was detected using a fluorescence polarization kit (IMAP FP IPP Explorer KIT, Molecular Device, Cat #R8124). The IMAP Reaction Buffer containing 0.1% BSA (5×) in the kit was diluted into a 1-fold reaction buffer containing 1 mM DTT. 0.12 nM PDE4B1 (BPS, Cat #60041) was added to the 1-fold reaction buffer to form a 2-fold enzyme solution. 10 μL of the 2-fold enzyme solution was added to wells of a 384-well reaction plate. For the negative control well, 10 μL of the 1-fold reaction buffer was used instead of the enzyme solution. Centrifugation at 1000 rpm for 1 minute and incubation at room temperature for 15 minutes were carried out. 0.2 uM FAM-labeled cAMP (FAM-cAMP, Molecular Device, Cat #R7506) was added to the 1-fold reaction buffer to form a 2-fold substrate solution. 10 μL of the 2-fold substrate solution was added to each well of the 384-well reaction plate. Centrifugation at 1000 rpm for 1 minute was carried out. A reaction was carried out at room temperature for 20 minutes. IMAP Progressive Binding Buffer A (5×), IMAP Progressive Binding Buffer B (5×), and IMAP Progressive Binding Reagent (provided by IMAP FP IPP Explorer Kit) were prepared into a reaction stop solution according to the instructions for use. 60 μL of an 80/60-fold reaction stop solution was added to each well of the 384-well reaction plate to stop the reaction, and the reaction product was centrifuged at 1000 rpm for 1 minute. Incubation at room temperature in the dark for 60 minutes was carried out. The mP values (Ex480/Em535 (s), Em535 (p)) were read by a microplate reader (Envision).

FP was usually represented by mP value. mP value for FP measurement=1000*(S−G*P)/(S+G*P)
S: fluorescence intensity in parallel direction
P: fluorescence intensity in perpendicular direction
G: G factor=1

### Calculation of Inhibition Rate (% Inhibition):

$$\% Inhibition = ( {mP}_{( sample )} - {mP}_{( sample )} ) / ( {mP}_{( positive control )} - {mP}_{( negative control )} ) ] \times 100 \%$$

mP₍ₛₐₘₚₗₑ₎: mP of the test compound in a reaction well
mP_{(negative control)}: mP in the negative control well
mP_{(positive control)}: mP in the positive control well

According to the calculated inhibition rate at each concentration, the IC50 value of each compound was calculated by XLFit excel add-in version 5.4.0.8 software.

$$Fitting formula : Y = Bottom + ( Top - Bottom ) / ( 1 + ( {IC}_{50} / X ) \hat{} HillSlope$$

The IC₅₀ values of the compounds of the present disclosure against PDE4B1 are less than 300 nM, and the IC₅₀ values of the compounds are preferably less than 100 nM, more preferably less than 50 nM, and further preferably less than 10 nM. The IC₅₀ values of some specific compounds are as shown in Table 3 below, wherein A<10 nM, 10 nM≤B<50 nM, and 50 nM≤C<100 nM.

TABLE 3
PDE4B1 activity

| Compound | PDE4B1 IC₅₀ (nM) |
|---|---|
| Compound 3 | A |
| Compound 8-2 | A |
| Compound 9-2 | A |
| Compound 10-2 | B |
| Compound 11-1 | A |

### 4. Effect of Compounds on PDE4D2 Activity

The effect of compounds on PDE4D2 activity was detected using a fluorescence polarization kit (BPS Bioscience, Catalog #60345). During the reaction, 12.5 μL of 0.272 ng/well of an enzyme (final concentration: 0.068 ng/well) and 12.5 μL of the compound diluted in a gradient manner (DMSO concentration: 4%) were first pre-incubated at room temperature for 15 minutes, the same volume of the enzyme at the same concentration and 12.5 μL of PDE buffer containing 4% DMSO were added to the positive control well, and 25 μL of PDE buffer containing 2% DMSO was added to the negative control well. After completion, 25 μL of 0.2 M FAM-Cyclic-3′,5′-AMP (final concentration: 0.1 μM) was added to each well, fully mixed, and then incubated at room temperature with slow shaking for 30 minutes. The Binding Agent was diluted with the Binding Agent Diluent (cAMP) at a ratio of 1:100 for later use, the Binding Agent dilution was taken and added to all the wells of the test plate at 100 μl/well and incubated at room temperature with slow shaking for 1 hour. After the incubation was complete, BMG LRBTECH microplate reader was used for FP detection, with Excitation at 485 nm and Emission at 520 nm. FP was usually represented by mP value.

$$mP = ( \frac{{I}_{II} - G ( {I}_{⊥} )}{{I}_{II} + G ( {I}_{⊥} )} ) \times 1000$$

I_{∥} (S520): fluorescence intensity in parallel direction
I_{⊥} (P520): fluorescence intensity in perpendicular direction
G: G factor=1

### Calculation of Inhibition Rate (% Inhibition):

$$\% Inhibition = [ 1 - ( {mP}_{( sample )} - {mP}_{( negative control )} ) / \\ ( {mP}_{( positive control )} - {mP}_{( negative control )} ) ] \times 100 \%$$

mP₍ₛₐₘₚₗₑ₎: mP of the test compound in a reaction well
mP_{(negative control)}: mP in the negative control well
mP_{(positive control)}: mP in the positive control well

According to the calculated inhibition rate at each concentration, the IC50 value of each compound was calculated by GraphPad Prism 8 software.

The IC₅₀ values of some specific compounds of the present disclosure are as shown in Table 4 below, wherein A<10 nM, 10 nM≤B<50 nM, and 50 nM≤C<100 nM.

TABLE 4
PDE4D2 activity

| | PDE4D2 IC₅₀ |
|---|---|
| Compound | (nM) |
| Compound 3 | A |
| Compound 8-2 | A |
| Compound 9-2 | A |
| Compound 11-1 | B |
| Compound 12-2 | B |
| Compound 13-2 | B |
| Compound 14-1 | B |
| Compound 15-2 | B |
| Compound 16-2 | B |
| Compound 17-2 | B |
| Compound 19-1 | B |
| Compound 23 | B |
| Compound 24-2 | B |
| Compound 26 | B |
| Compound 30 | A |
| Compound 31 | B |
| Compound 32-2 | A |
| Compound 34 | B |
| Compound 35 | A |
| Compound 36-1 | A |
| Compound 36-2 | B |
| Compound 39 | A |
| Compound 41 | B |
| Compound 43 | A |
| Compound 44 | B |
| Compound 45-1 | A |
| Compound 45-2 | B |
| Compound 46-1 | A |
| Compound 46-2 | B |
| Compound 47 | A |
| Compound 48 | A |
| Compound 49 | A |
| Compound 50 | A |
| Compound 51 | A |
| Compound 52 | A |
| Compound 56 | A |
| Compound 57 | B |
| Compound 58 | B |
| Compound 63 | A |
| Compound 64 | A |
| Compound 65 | A |
| Compound 66 | A |
| Compound 69 | A |
| Compound 70 | A |
| Compound 71-3 | B |
| Compound 71-4 | A |
| Compound 72-3 | B |
| Compound 72-4 | A |
| Compound 73-1 | A |
| Compound 73-2 | B |
| Compound 74-2 | A |
| Compound 75-1 | B |
| Compound 76-1 | B |
| Compound 77-1 | A |
| Compound 77-3 | A |
| Compound 78 | B |
| Compound 79 | A |
| Compound 80-2 | B |
| Compound 81-1 | A |
| Compound 82-2 | B |
| Compound 83 | B |
| Compound 84 | A |
| Compound 85-1 | A |
| Compound 88 | A |
| Compound 89-2 | B |
| Compound 90 | B |
| Compound 91-1 | A |
| Compound 92-1 | A |
| Compound 92-2 | B |
| Compound 93-1 | A |
| Compound 94-1 | A |
| Compound 94-2 | B |
| Compound 95-1 | A |
| Compound 95-2 | A |
| Compound 95-3 | A |
| Compound 95-4 | A |
| Compound 96-1 | B |
| Compound 96-2 | A |
| Compound 96-3 | B |
| Compound 96-4 | B |
| Compound 97 | A |
| Compound 98 | A |
| Compound 100 | A |
| Compound 101 | A |
| Compound 102-1 | C |
| Compound 102-2 | A |
| Compound 103-2 | A |
| Compound 104 | B |
| Compound 105 | A |
| Compound 106 | B |
| Compound 107 | B |
| Compound 108 | B |
| Compound 109-2 | C |
| Compound 109-4 | A |
| Compound 110-1 | A |
| Compound 110-2 | B |
| Compound 111-2 | A |
| Compound 111-4 | A |
| Compound 112-1 | B |
| Compound 112-2 | C |
| Compound 113-2 | C |
| Compound 114-1 | B |
| Compound 115-1 | C |
| Compound 116 | C |
| Compound 117 | B |
| Compound 118 | B |
| Compound 119 | B |
| Compound 121-1 | B |
| Compound 121-2 | B |
| Compound 122 | B |
| Compound 123-1 | A |
| Compound 123-2 | A |
| Compound 124 | B |
| Compound 125 | B |
| Compound 126 | B |
| Compound 127-1 | B |
| Compound 127-2 | B |
| Compound 128 | A |
| Compound 129 | B |
| Compound 130 | B |
| Compound 131-1 | B |
| Compound 131-2 | C |
| Compound 132 | B |
| Compound 133-1 | B |
| Compound 133-2 | A |
| Compound 134 | A |
| Compound 135 | A |
| Compound 136 | A |
| Compound 137 | B |
| Compound 138 | B |
| Compound 139-1 | A |
| Compound 139-2 | B |
| Compound 140-1 | B |
| Compound 140-2 | C |
| Compound 141-1 | B |
| Compound 141-2 | C |
| Compound 142-1 | B |
| Compound 142-2 | B |
| Compound 143-3 | A |
| Compound 143-4 | A |
| Compound 144-1 | B |
| Compound 144-2 | A |
| Compound 144-4 | A |
| Compound 145-1 | A |
| Compound 145-4 | A |
| Compound 146-1 | A |
| Compound 146-2 | B |
| Compound 147-1 | C |
| Compound 147-2 | C |

5. Detection of Inhibitory Activity of Compounds on the Release of Tumor Necrosis Factor-α (TNF-α) from Human Peripheral Blood Mononuclear Cells Induced by Lipopolysaccharide (LPS) In Vitro

Normal human peripheral blood which was anticoagulated (citric acid anticoagulation) was collected, and hPBMCs were prepared by Ficoll-Paque PLUS (Cytiva, Cat #17144002, density 1.077 g/mL). The concentration of hPBMC cells was adjusted to 0.25×10⁶ cells/mL with RPMI1640 medium and inoculated into a 96-well plate in an amount of 50000 cells per well. Subsequently, different concentrations of drugs were added for pre-incubation for 1 h (the DMSO final concentration was 0.1%, and the positive and negative control wells had the same volume of RPMI1640 containing 0.1% DMSO). After the pre-incubation was complete, 100 ng/mL LPS (SIGMA, L2630) was added to the compound and positive control wells, and the negative control well had the same volume of RPMI1640 containing 0.1% DMSO. Incubation was carried out for 4 h in an incubator at 37° C. and 5% CO₂. The cell supernatant was collected, and the content of TNF-α in the supernatant sample was detected by human TNF-α Elisa quantitative test kit (Sino Biological, Cat #KIT10602). The IC₅₀ value was calculated using GraphPad Prism software. The IC₅₀ values of some specific compounds of the present disclosure are as shown in Table 5 below, wherein A<10 nM, 10 nM≤B<50 nM, and 50 nM≤C<100 nM.

TABLE 5
Inhibitory activity of compounds
on TNF-α induced by LPS in vitro

| Test compound | Re IC₅₀ (nM) | Max inhibition (%) |
|---|---|---|
| Compound 9-2 | B | 69.47 |
| Compound 32-2 | A | / |
| Compound 35 | A | 85.08 |
| Compound 36-1 | A | 73.52 |
| Compound 37 | B | 78.28 |
| Compound 39 | A | 71.11 |
| Compound 41 | B | 69.8 |
| Compound 43 | B | 80.31 |
| Compound 44 | B | 77.27 |
| Compound 45-1 | B | 71.28 |
| Compound 46-1 | B | 75.89 |
| Compound 46-2 | B | 73.36 |
| Compound 47 | A | 80.45 |
| Compound 48 | B | 73.86 |
| Compound 49 | A | 73.46 |
| Compound 50 | A | 72.14 |
| Compound 51 | A | 69.04 |
| Compound 52 | B | / |
| Compound 56 | A | / |
| Compound 60-2 | B | 72.31 |
| Compound 63 | B | 72.71 |
| Compound 64 | B | 79.89 |
| Compound 65 | B | 68.15 |
| Compound 66 | B | 67.07 |
| Compound 69 | A | 70.87 |
| Compound 70 | A | 55.38 |
| Compound 71-2 | B | 58.51 |
| Compound 71-3 | A | 61.86 |
| Compound 71-4 | A | 57.63 |
| Compound 72-3 | A | 65.66 |
| Compound 72-4 | A | 62.45 |
| Compound 73-1 | A | 67.49 |
| Compound 73-2 | A | 67.88 |
| Compound 74-1 | A | 51.67 |
| Compound 74-2 | A | 79.64 |
| Compound 76-1 | B | 74.65 |
| Compound 77-1 | A | 65.28 |
| Compound 77-3 | A | 66.35 |
| Compound 78 | B | 77.39 |
| Compound 79 | B | 65.52 |
| Compound 80-2 | A | 75.43 |
| Compound 81-1 | A | 74.08 |
| Compound 82-2 | B | 73.50 |
| Compound 83 | A | 71.03 |
| Compound 84 | A | 68.04 |
| Compound 85-1 | A | 71.38 |
| Compound 88 | A | 74.9 |
| Compound 90 | B | 69.53 |
| Compound 91-1 | A | 72.87 |
| Compound 92-1 | A | 69.72 |
| Compound 93-1 | A | 71.65 |
| Compound 94-1 | A | 76.10 |
| Compound 94-2 | A | 67.98 |
| Compound 95-2 | A | 70.67 |
| Compound 95-3 | A | 81.97 |
| Compound 96-1 | A | 72.83 |
| Compound 96-2 | A | 81.63 |
| Compound 96-4 | B | 70.23 |
| Compound 97 | A | 76.25 |
| Compound 98 | A | 72.70 |
| Compound 100 | A | 79.21 |
| Compound 101 | A | 79.98 |
| Compound 102-1 | B | 99.73 |
| Compound 102-2 | A | 87.54 |
| Compound 104 | B | 78.75 |
| Compound 105 | A | 83.96 |
| Compound 106 | B | 98.36 |
| Compound 109-4 | A | 85.74 |
| Compound 110-1 | A | 88.09 |
| Compound 110-2 | A | 74.07 |
| Compound 111-2 | A | 75.39 |
| Compound 111-4 | A | 78.77 |
| Compound 112-1 | B | 83.76 |
| Compound 117 | B | 85.97 |
| Compound 119 | B | 84.64 |
| Compound 121-1 | A | 78.80 |
| Compound 139-1 | A | 92.69 |
| Compound 140-1 | A | 93.38 |
| Compound 140-2 | B | 89.58 |
| Compound 144-4 | A | 94.06 |

Re IC₅₀: relative IC₅₀.

### 6. Pharmacokinetic Test in Mice

6.1 Experimental animals: Male ICR mice, 20-25 g, 6 mice/compound, purchased from Chengdu Ddossy Experimental Animals Co., Ltd.

6.2 Experiment design: On the day of the experiment, 6 ICR mice were randomly grouped according to their body weights. The animals were fasted with water available for 12 to 14 h one day before the administration and were fed 4 h after the administration.

TABLE 6
Administration information

| | | Administration information | | | | | |
|---|---|---|---|---|---|---|---|
| | | | Administration | Administration | Administration | | |
| | Number | Test | dosage | concentration | volume | Collected | Mode of |
| Group | Male | compound | (mg/kg) | (mg/mL) | (mL/kg) | sample | administration |
| G1 | 3 | Compound | 2.5 | 0.5 | 5 | Plasma | Intravenous |
| | | 51 | | | | | administration |
| G2 | 3 | | 10 | 1 | 10 | Plasma | Intragastric |
| | | | | | | | administration |

Note:
Vehicle I for intravenous administration: 5% DMA + 5% Solutol + 90% Saline; vehicle for intragastric administration: 0.5% MC (DMA: dimethylacetamide; Solutol: polyethylene glycol-15-hydroxystearate; Saline: physiological saline; MC: methylcellulose)
Before and after administration, 0.06 mL of blood was taken from the orbit under isoflurane anesthesia and placed in an EDTAK2 centrifuge tube for centrifugation at 5000 rpm at 4° C. for 10 min, followed by plasma collection. The blood collection time points for the intravenous group and intragastric group were both 0 min, 5 min, 15 min, 30 min, 1 h, 2 h, 4 h, 6 h, 8 h, and 24 h. Before analysis and detection, all samples were stored at −80° C., and the samples were quantitatively analyzed by LC-MS/MS.

TABLE 7
Pharmacokinetic parameters of test compounds in mouse plasma

| Test | Mode of | CL | Vdₛₛ | AUC₀₋ₜ | T½ | F |
|---|---|---|---|---|---|---|
| compound | administration | (mL/min/kg) | (L/kg) | (hr*ng/mL) | (h) | (%) |
| Compound | i.v. | 7.81 ± 1.4 | 1.29 ± 0.039 | 5423 ± 926 | 2.82 ± 0.23 | — |
| 51 | (2.5 mg/kg) | | | | | |
| | i.g. | — | — | 30077 ± 5361 | 3.33 ± 0.58 | >98 |
| | (10 mg/kg) | | | | | |

—: not applicable.

Conclusion: The compounds of the present disclosure, such as those in the examples, exhibited excellent pharmacokinetic properties in the PK test in mice.

### 7 Pharmacokinetic Test in Rats

7.1 Experimental animals: Male SD rats, about 220 g, 6-8 weeks old, 6 rats/compound, purchased from Chengdu Ddossy Experimental Animals Co., Ltd.

7.2 Experiment design: On the day of the experiment, 6 SD rats were randomly grouped according to their body weights. The animals were fasted with water available for 12 to 14 h one day before the administration and were fed 4 h after the administration.

TABLE 8
Administration information

| | | Administration information | | | | | |
|---|---|---|---|---|---|---|---|
| | | | Administration | Administration | Administration | | |
| | Number | Test | dosage | concentration | volume | Collected | Mode of |
| Group | Male | compound | (mg/kg) | (mg/mL) | (mL/kg) | sample | administration |
| G1 | 3 | Compound | 2.5 | 0.5 | 5 | Plasma | Intravenous |
| | | 84 | | | | | administration |
| G2 | 3 | | 10 | 1 | 10 | Plasma | Intragastric |
| | | | | | | | administration |

Note:
Vehicle for intravenous administration: 10% DMA + 10% Solutol + 80% Saline; vehicle for intragastric administration: 0.5% MC (DMA: dimethylacetamide; Solutol: polyethylene glycol-15-hydroxystearate; Saline: physiological saline; MC: methylcellulose)
Before and after the administration, 0.15 ml of blood was taken from the orbit under isoflurane anesthesia and placed in an EDTAK2 centrifuge tube for centrifugation at 5000 rpm at 4° C. for 10 min, followed by plasma collection. The blood collection time points for the intravenous administration group and intragastric administration group were: 0, 5 min, 15 min, 30 min, 1 h, 2 h, 4 h, 6 h, 8 h and 24 h. Before analysis and detection, all samples were stored at −80° C., and the samples were quantitatively analyzed by LC-MS/MS.

TABLE 9
Pharmacokinetic parameters of test compounds in rat plasma

| Test | Mode of | CL | Vdₛₛ | AUC₀₋ₜ | |
|---|---|---|---|---|---|
| compound | administration | (mL/min/kg) | (L/kg) | (hr*ng/mL) | F (%) |
| Compound | i.v. (2.5 | 2.54 ± 0.32 | 0.324 ± 0.012 | 16531 ± | — |
| 84 | mg/kg) | | | 1991 | |
| | i.g. (10 | — | — | 52179 ± | 78.9 ± 32 |
| | mg/kg) | | | 21323 | |

—: not applicable.

Conclusion: The compounds of the present disclosure, such as those in the examples, exhibited excellent pharmacokinetic properties in the PK test in rat.

### 8. Pharmacokinetic Test in Beagle Dogs

8.1 Experimental animals: Male beagle dogs, about 8-11 kg, 6 beagle dogs/compound, purchased from Beijing Marshall Biotechnology Co., Ltd.

8.2 Experimental method: On the day of the experiment, 6 beagle dogs were randomly grouped according to their body weights. The animals were fasted with water available for 12 to 14 h one day before the administration and were fed 4 h after the administration. Administration was carried out according to Table 10.

TABLE 10
Administration information

| | | Administration information | | | | | |
|---|---|---|---|---|---|---|---|
| | | | Administration | Administration | Administration | | |
| | Number | Test | dosage | concentration | volume | Collected | Mode of |
| Group | Male | compound | (mg/kg) | (mg/mL) | (mL/kg) | sample | administration |
| G1 | 3 | Compound | 1 | 1 | 1 | Plasma | Intravenous |
| | | 51 | | | | | administration |
| G2 | 3 | | 3 | 0.6 | 5 | Plasma | Intragastric |
| | | | | | | | administration |

Note:
Vehicle I for intravenous administration: 5% DMA + 5% Solutol + 90% Saline; vehicle for intragastric administration: 0.5% MC (DMA: dimethylacetamide; Solutol: polyethylene glycol-15-hydroxystearate; Saline: physiological saline; MC: Methylcellulose solution)
Before and after the administration, 1 ml of blood was taken from the jugular veins or limb veins, and placed in an EDTAK2 centrifuge tube. Centrifugation was performed at 5000 rpm at 4° C. for 10 min, and plasma was collected. The blood collection time points for the intravenous group and intragastric administration group were: 0 min, 5 min, 15 min, 30 min, 1 h, 2 h, 4 h, 6 h, 8 h, 10 h, 12 h, 24 h, 48 h, and 72 h. Before analysis and detection, all samples were stored at −80° C., and the samples were quantitatively analyzed by LC-MS/MS.

TABLE 11
Pharmacokinetic parameters of test compounds in beagle dog plasma

| Test | Mode of | CL | Vdₛₛ | AUC₀₋ₜ | T½ | F |
|---|---|---|---|---|---|---|
| compound | administration | (mL/min/kg) | (L/kg) | (hr*ng/mL) | (h) | (%) |
| Compound | i.v. | 3.06 ± 0.18 | 3.08 ± 0.61 | 4991 ± 239 | 12.8 ± 3.8 | — |
| 51 | (1 mg/kg) | | | | | |
| | i.g. | — | — | 11211 ± 1713 | 16.1 ± 5.5 | 74.9 ± 11 |
| | (3 mg/kg) | | | | | |

—: not applicable.

Conclusion: The compounds of the present disclosure, such as those in the examples, exhibited excellent pharmacokinetic properties in the PK test in beagle dogs.

### 9. Detection of Inhibitory Activity on TNF-α Secretion in LPS-Induced Lung Inflammation Model of Mice

In mice, intratracheal administration of lipopolysaccharides (LPS, Sigma, L2880) might induce increased TNF-α concentration in lung tissue. In this experiment, a certain number of mice (BABL/c, male, weighing 18-22 g) were randomly divided into groups, with 8 mice in each group, and orally administered with a certain dose of the test compound (the test compound was suspended with 0.5% MC and prepared into a certain concentration, and the suspension was orally administered at a volume of 10 ml/kg body weight). After 0.5 h, based on 2.5 uL/g animal body weight, LPS was intratracheally administered at a dose of 0.8 mg/mL via a lung quantitative atomizing needle. 24 h later, the mice were intraperitoneally anesthetized with 20% urethane (10 mL/kg), and then sacrificed by cervical dislocation. Subsequently, lung tissue was taken and the lower part of the left lobe was fully homogenized, and the content of TNF-α in the supernatant of the homogenate was detected by Mouse TNFα ELISA Kit (RD, SMTA00B).

$$Inhibition rate of test compound on TNF - α level ( \% Inh ) = ( model group - test group ) / model group * 100 \% .$$

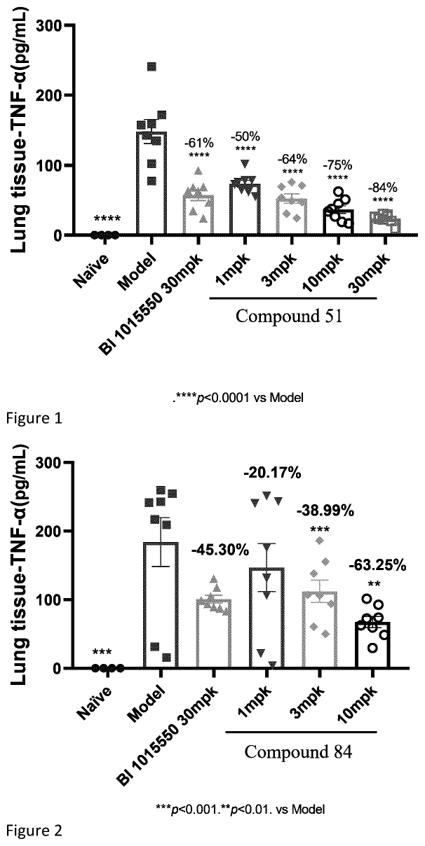

Results and conclusion: The results are shown in FIGS. 1 and 2.

1. A compound represented by formula I, or a stereoisomer or pharmaceutically acceptable salt thereof, 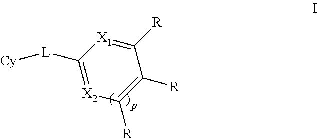
wherein when Cy is selected from Cy1, Cy2, Cy3, Cy8, and Cy10, L is -(L₁)n-(L₂)m-; 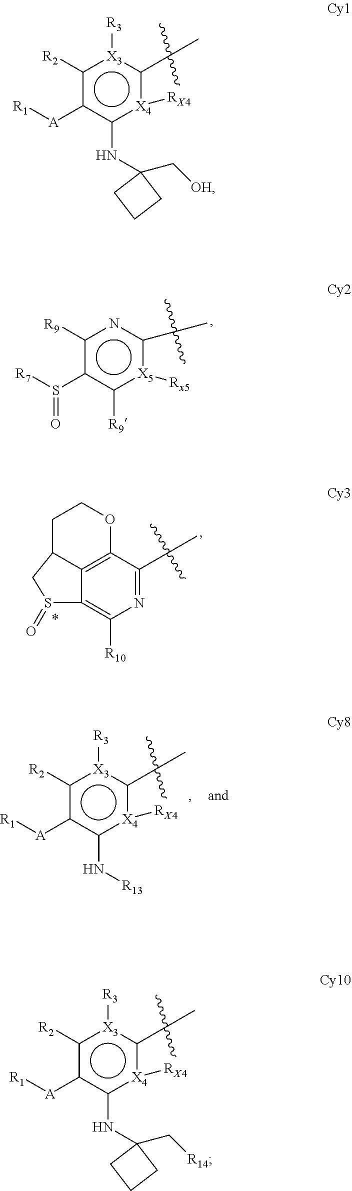
when Cy is selected from Cy4, Cy5, Cy6, and Cy11, L is -L₃-; 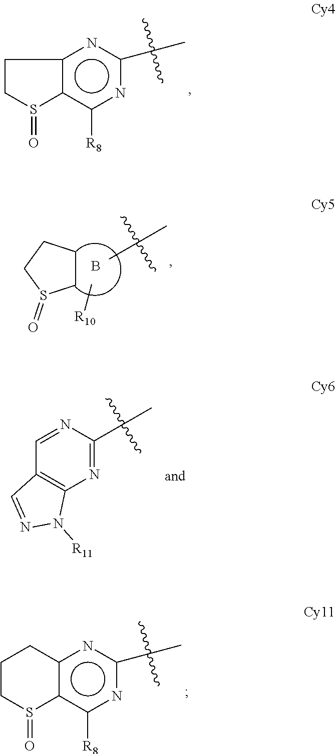
and
when Cy is selected from Cy7 and Cy9, L is -L₄-; 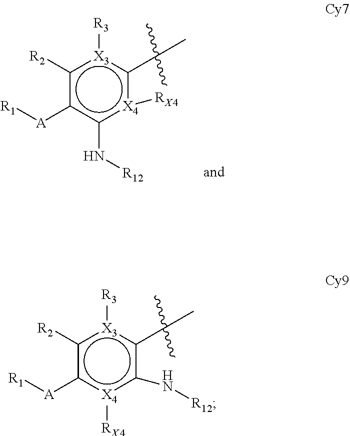
L₁ is —NR₄—, —CR₅=N—, —CR₅=CR₅—, or —CR₅R₅—;
L₂ is 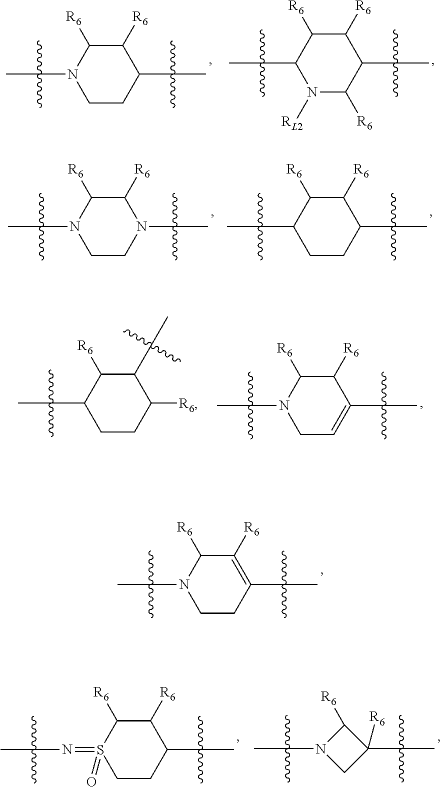
C₁₋₄ alkylene, C₂₋₄ alkenylene, C₂₋₄ alkynylene, CO, O, NR_{L2}, a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 10-membered fused heterocycloalkyl containing 1-3 heteroatoms selected from N, S, and O, a 6- to 10-membered bridged heterocycloalkyl containing 1-3 heteroatoms selected from N, S, and O, or a 7- to 12-membered spiro heterocycloalkyl containing 1-3 heteroatoms selected from N, S, and O, wherein the alkylene, alkenylene, alkynylene, monocyclic heterocycloalkyl, fused heterocycloalkyl, bridged heterocycloalkyl, and spiro heterocycloalkyl are optionally substituted with 1-3 groups selected from halogen, ═O, CN, C₁₋₄ alkyl, C₁₋₄ alkoxy, OH, and NH₂;
R_{L2} is H, C₁₋₄ alkyl, or C₃₋₆ cycloalkyl;
L₃ is heterocycloalkylene or heterocycloalkylene-(CH₂)ᵣ—, wherein the heterocycloalkylene is a 4- to 10-membered heterocycle containing 1-3 heteroatoms selected from N, S, and O and is optionally substituted with 1-3 R_{L3};
each R_{L3} is independently selected from halogen, ═O, CN, C₁₋₄ alkyl, or C₁₋₄ alkoxy, or R_{L3} and R_{X1}, together with the atom to which they are attached, form C₃₋₆ cycloalkyl or a 5- to 8-membered heterocycloalkyl containing 1-3 heteroatoms selected from N, S, and O;
L₄ is 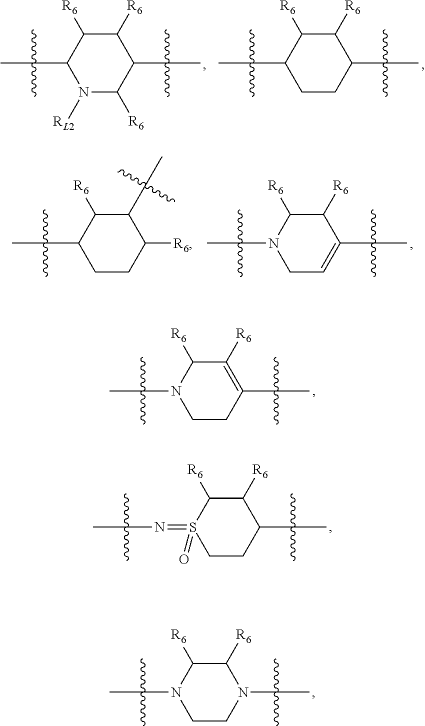
a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 6-membered heterocycloalkyl fused 5- to 6-membered heterocycloalkyl containing 1-3 heteroatoms selected from N, S, and O, a 5- to 6-membered heterocycloalkyl fused 5- to 6-membered cycloalkyl containing 1-3 heteroatoms selected from N, S, and O, or a 7- to 12-membered spiro heterocycloalkyl containing 1-3 heteroatoms selected from N, S, and O, wherein the monocyclic heterocycloalkyl, 5- to 6-membered heterocycloalkyl fused 5- to 6-membered heterocycloalkyl, 5- to 6-membered heterocycloalkyl fused 5- to 6-membered cycloalkyl, and spiro heterocycloalkyl are optionally substituted with 1-3 groups selected from halogen, ═O, CN, C₁₋₄ alkyl, C₁₋₄ alkoxy, OH, and NH₂;
A is —S(O)—, —C(O)—, —B(OH)—, —S—, —S(═N)—CN, or —C(═N)—CN;
X₁ and X₂ are independently CR_{X1}, O, S, or N;
X₃, X₄, and X₅ are independently C or N;
ring B is heteroaryl or heterocycloalkyl fused heteroaryl, wherein the heteroaryl is a 5-membered heteroaryl containing 1-3 heteroatoms selected from N, S, and O, and the heterocycloalkyl is a 5- to 6-membered heterocycloalkyl containing 1-3 heteroatoms selected from N, S, and O;
each R_{X1} and R are independently H, halogen, C₁₋₄ alkyl, C₁₋₄ alkoxy, —N(CH₃)₂, —NH(CH₃), C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, O, and Si, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₁ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₁ and R₂, together with the atom to which they are attached, form a 5- to 10-membered heterocycloalkyl or a 5- to 6-membered heteroaryl containing 1-3 heteroatoms selected from N, S, B, and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
R₂ is H, C₁₋₄ alkyl, or C₁₋₄ haloalkyl;
R₃ is H, halogen, C₁₋₄ alkyl, CN, OH, or absent, or R₃ and R₂, R₃ and R₆, R₃ and R₄, or R₃ and R₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent, or Rₓ₄ and R₄, Rₓ₄ and R₅, or Rₓ₄ and R₆, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the alkyl, cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₄ is H, C₁₋₄ alkyl, C₁₋₄ haloalkyl, or C₃₋₆ cycloalkyl;
R₅ is H, C₁₋₄ alkyl, C₁₋₄ haloalkyl, C₁₋₄ alkoxy, —NHC₁₋₄ alkyl, or C₃₋₆ cycloalkyl;
R₆ is H, halogen, ═O, CN, C₁₋₄ alkyl, or C₁₋₄ alkoxy;
Rₓ₅ is C₁₋₄ alkyl, CN, OH, or absent;
R₇ is C₁₋₄ alkyl, C₁₋₄ haloalkyl, C₃₋₆ cycloalkyl, or a 5- to 8-membered heterocycloalkyl containing 1-3 heteroatoms selected from N, S, and O, or R₇ and R₉, together with the atom to which they are attached, form a 5- to 6-membered heterocycloalkyl containing 1-3 heteroatoms selected from N, S, and O, wherein the alkyl, haloalkyl, cycloalkyl, and heterocycloalkyl are optionally substituted with 1-3 groups selected from halogen, CN, C₁₋₄ alkyl, C₁₋₄ alkoxy, OH, and NH₂;
at least one group of R_{9′} and R₇ or R_{9′} and Rₓ₅, together with the atom to which they are attached, form C₃₋₈ cycloalkyl, phenyl, a 5- to 10-membered heterocycloalkyl containing 1-3 heteroatoms selected from N, S, and O, or a 5- to 6-membered heteroaryl containing 1-3 heteroatoms selected from N, S, and O, wherein the cycloalkyl, phenyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′;}
R₈ is R_{8′}, —OR_{8′}, or —(CH₂)ᵣR_{8′};
R_{8′} is C₃₋₆ cycloalkyl, a 4- to 8-membered heterocycloalkyl containing 1-3 heteroatoms selected from N, S, O, Si, and P, a 5- to 6-membered heteroaryl containing 1-3 heteroatoms selected from N, S, and O, —N═S(═O)(C₁₋₄ alkyl)₂, or —N(C₃₋₆ cycloalkyl)C(O)NH(C₁₋₄ alkyl), wherein the cycloalkyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, and CN;
R₉ is H, C₁₋₄ alkyl, or C₁₋₄ haloalkyl;
each R₁₀ is independently R_{8′}, —OR_{8′}, —NHR_{8′}, or —(CH₂)ᵣR_{8′};
R₁₁ is C₃₋₆ cycloalkyl optionally substituted with 1-3 groups selected from halogen, CN, ═O, OH, C₁₋₄ alkyl, hydroxy C₁₋₄ alkyl, and C₁₋₄ haloalkyl;
R₁₂ is selected from a 4- to 10-membered heterocycloalkylene, a 3- to 10-membered cycloalkylene, a 6- to 10-membered arylene, a 5- to 10-membered heteroarylene, hydroxy-C₁₋₆ alkyl, C₁₋₄ alkyl-CN, —CRaRb=N—O—C₁₋₄ alkyl, —C(NRaRb)=N—CN, —C(C₁₋₄ alkyl)=N—CN, —C(NRaRb)=CRa—NO₂, —C(CH₃)₂—CH₂—O—C(O)NH₂, —C(CH₃)₂—CH₂—NH—C(O)O(C₁₋₄ alkyl), —C(CH₃)₂—CH₂—O—C(O)NHCH₃, —CH(CH₃)—CH₂—O—C(O)NH₂, —CH(CH₃)—CH₂—NH—C(O)O(C₁₋₄ alkyl), —CH(CH₃)—CH₂—O—C(O)NHCH₃, —C(O)—Ra, or —C(O)-(4- to 6-membered heterocycloalkyl), wherein the heterocycloalkylene, cycloalkylene, arylene, and heteroarylene are optionally further substituted with 1-3 groups selected from halogen, CN, ═O, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NRa—C(O)NH₂, —(CH₂)ₜ—NRa—C(═NH)NH₂, —(CH₂)ₜ—NRa—C(O)—O—(C₁₋₄ alkyl), —(CH₂)ₜ—NRa—C(C₁₋₄ alkyl)=N—CN, —(CH₂)ₜ—NRa—C(NRaRb)=N—CN, —(CH₂)ₜ—NRa—C(NRaRb)=CRa—NO₂, —(CH₂)ₜ—C(NRaRb)=N—CN, —(CH₂)ₜ—C(C₁₋₄ alkyl)=N—CN, —(CH₂)ₜ—C(C₁₋₄ alkyl)=N—O—(C₁₋₄ alkyl), ═N—O—C₁₋₄ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃;
Ra and Rb are each independently selected from H, halogen, C₁₋₄ alkyl, C₁₋₄ haloalkyl, C₁₋₄ alkoxy, halo C₁₋₄ alkoxy, or a 4- to 6-membered heterocycloalkyl containing 1-2 heteroatoms selected from N and 0;
or alternatively, Ra and Rb, together with the atom to which they are attached, form C₃₋₆ cycloalkyl;
R₁₃ is selected from a 7- to 11-membered spiro ring, a 4- to 10-membered fused ring, a 5- to 8-membered bridged heterocycle, a 4- to 6-membered monocyclic heterocycloalkyl containing S(O)₂ group, a 4-membered monocyclic heterocycloalkyl containing 1-3 heteroatoms selected from N, S, and O, and a 7-membered monocyclic heterocycloalkyl containing 1-3 heteroatoms selected from N, S, and O, wherein the spiro ring, fused ring, bridged heterocycle, and monocyclic heterocycloalkyl are optionally substituted with 1-3 groups selected from halogen, CN, ═O, OH, C₁₋₄ alkyl, hydroxy C₁₋₄ alkyl, and C₁₋₄ haloalkyl;
R₁₄ is selected from —(CH₂)ₜ—O—C(O)NH₂, —(CH₂)ₜ—O—C(O)NHCH₃, and a 5- to 6-membered monocyclic heterocycloalkyl containing 1-3 heteroatoms selected from N, S, and O, wherein the monocyclic heterocycloalkyl is optionally substituted with 1-3 groups selected from halogen, CN, ═O, OH, C₁₋₄ alkyl, and C₁₋₄ haloalkyl;
n and m are independently 0, 1, 2, or 3;
r is selected from 1, 2, 3, or 4;
t is selected from 0, 1, 2, 3, or 4; and
p is selected from 0 or 1;
provided that
Cy1 is not

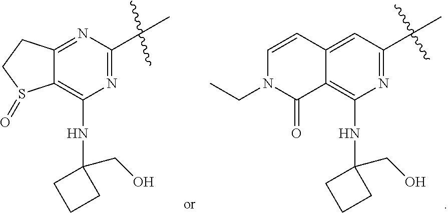 [SMILES: *c1nc2c(c(NC3(CO)CCC3)n1)S(=O)CC2 ; *c1cc2ccn(CC)c(=O)c2c(NC2(CO)CCC2)n1]

2. The compound or the stereoisomer or pharmaceutically acceptable salt thereof according to claim 1,
wherein ring B is pyrazolyl, imidazolyl, pyrrolyl, tetrahydropyrrolopyrrolyl, tetrahydropyrroloimidazolyl, or thienopyrrolyl;
R₁ is C₁₋₄ alkyl, or C₁₋₄ haloalkyl, or R₁ and R₂, together with the atom to which they are attached, form a 5- to 7-membered monocyclic heterocycloalkyl, a 6- to 8-membered fused heterocycloalkyl, a 7- to 9-membered spiro heterocycloalkyl, or a 5- to 6-membered heteroaryl containing 1-3 heteroatoms selected from N, S, B, and O, wherein the monocyclic heterocycloalkyl, fused heterocycloalkyl, spiro heterocycloalkyl, and heteroaryl are optionally substituted with 1-3 groups selected from halogen, ═O, CN, C₁₋₄ alkyl, C₁₋₄ alkoxy, OH, and NH₂;
R₂ is H, C₁₋₄ alkyl, or C₁₋₄ haloalkyl;
R₃ is H, halogen, C₁₋₄ alkyl, CN, OH, or absent;
or R₃ and R₂, together with the atom to which they are attached, form a 5- to 6-membered heterocycloalkyl or a 5- to 6-membered heteroaryl containing 1-3 heteroatoms selected from N, S, and O, wherein the heterocycloalkyl and heteroaryl are optionally substituted with 1-3 groups selected from halogen, ═O, CN, C₁₋₄ alkyl, C₁₋₄ alkoxy, OH, and NH₂;
or R₃ and R₄ or R₃ and R₅, together with the atom to which they are attached, form a 5- to 6-membered heterocycloalkyl or a 5- to 6-membered heteroaryl containing 1-3 heteroatoms selected from N, S, and O, or phenyl, wherein the heterocycloalkyl, phenyl, and heteroaryl are optionally substituted with 1-3 groups selected from halogen, ═O, CN, C₁₋₄ alkyl, C₁₋₄ alkoxy, OH, and NH₂;
or R₃ and R₆, together with the atom to which they are attached, form C₅₋₇ cycloalkyl, a 5- to 6-membered heterocycloalkyl, or a 5- to 6-membered heteroaryl containing 1-3 heteroatoms selected from N, S, and O, wherein the cycloalkyl, heterocycloalkyl, and heteroaryl are optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent, or Rₓ₄ and R₄ or Rₓ₄ and R₅, together with the atom to which they are attached, form a 5- to 6-membered heteroaryl containing 1-3 heteroatoms selected from N, S, and O, wherein the heteroaryl is optionally substituted with 1-3 groups selected from halogen, ═O, CN, C₁₋₄ alkyl, C₁₋₄ alkoxy, OH, and NH₂;
R₇ is C₁₋₄ alkyl, or C₁₋₄ haloalkyl, or R₇ and R₉, together with the atom to which they are attached, form a 5- to 6-membered heterocycloalkyl containing 1-3 heteroatoms selected from N, S, and O, wherein the heterocycloalkyl is optionally substituted with 1-3 groups selected from halogen, CN, C₁₋₄ alkyl, C₁₋₄ alkoxy, OH, and NH₂;
at least one group of R_{9′} and R₇ or R_{9′} and Rₓ₅, together with the atom to which they are attached, form a 5- to 7-membered monocyclic heterocycloalkyl, a 7- to 10-membered spiro heterocycloalkyl, or a 5- to 6-membered heteroaryl containing 1-3 heteroatoms selected from N, S, and O, wherein the monocyclic heterocycloalkyl, spiro heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′}; and
r is 1 or 2.

3. The compound or the stereoisomer or pharmaceutically acceptable salt thereof according to claim 2,
wherein each L₂ is independently selected from 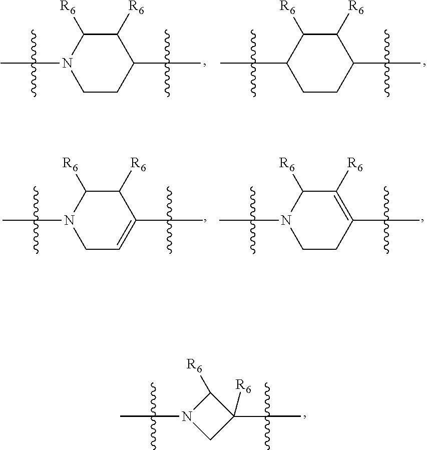
CO, O, NR_{L2}, C₁₋₄ alkylene, C₂₋₄ alkenylene, C₂₋₄ alkynylene, 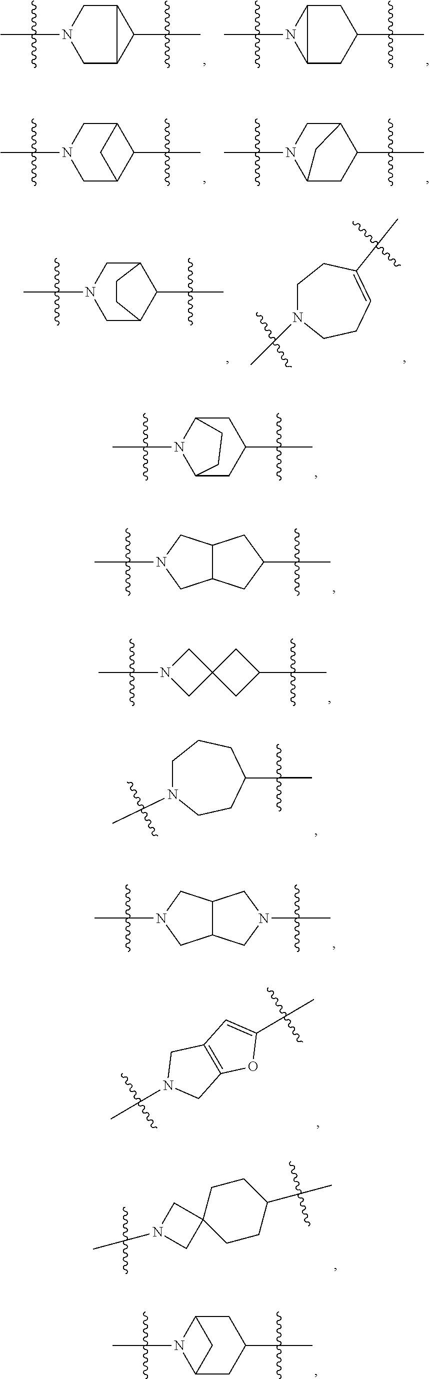 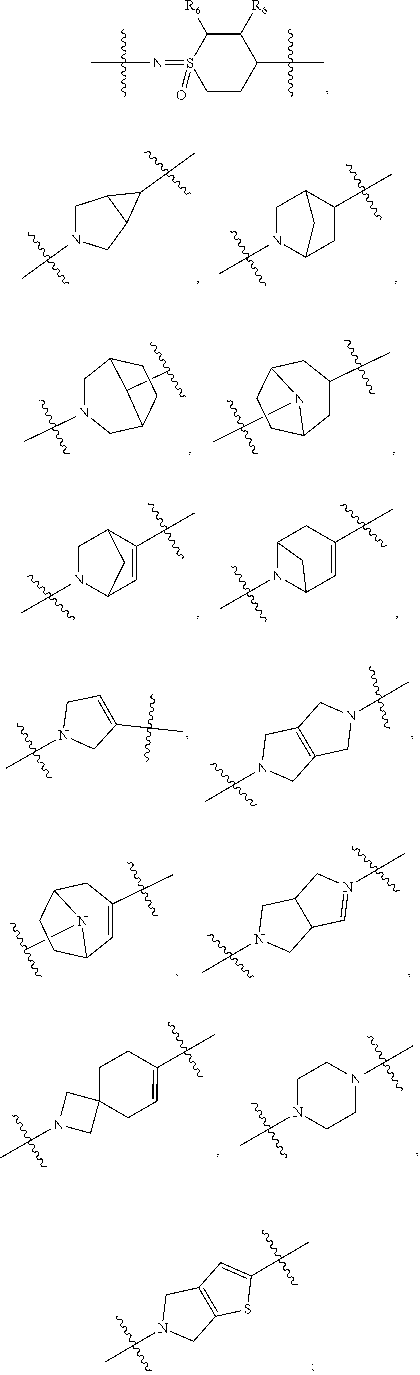
R_{L2} is H, C₁₋₄ alkyl, or C₃₋₆ cycloalkyl;
L₃ is selected from 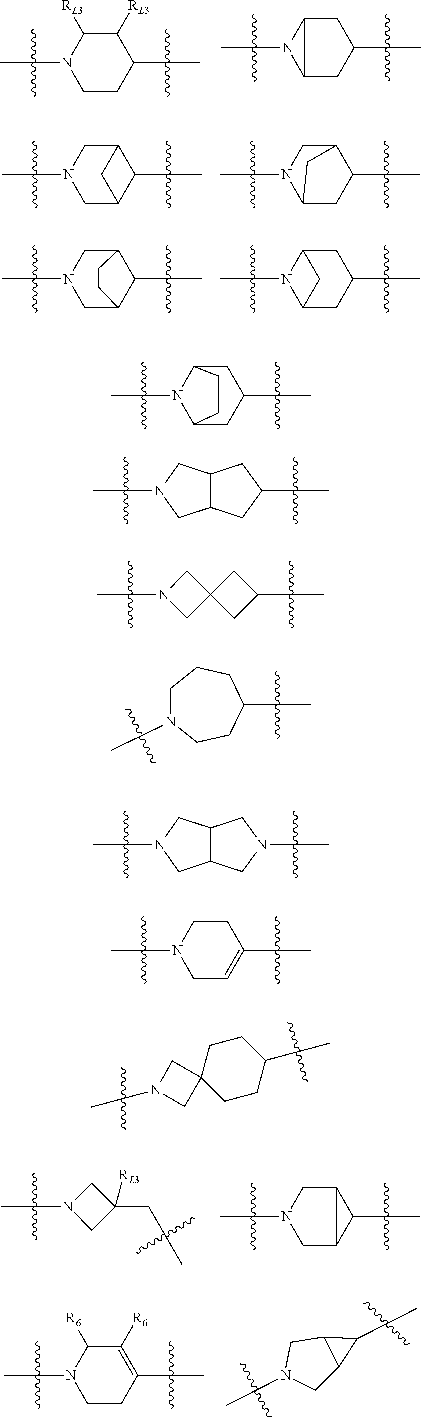 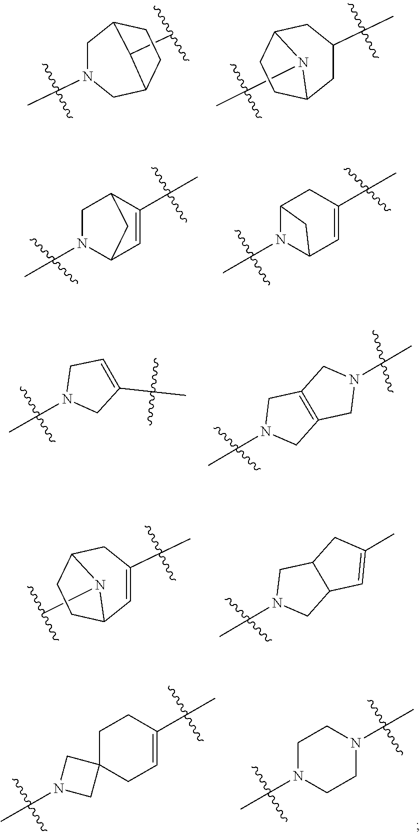
each R_{L3} is independently selected from halogen, ═O, CN, C₁₋₄ alkyl, or C₁₋₄ alkoxy, or R_{L3} and R_{X1}, together with the atom to which they are attached, form C₃₋₆ cycloalkyl or a 5- to 6-membered heterocycloalkyl containing 1-3 heteroatoms selected from N, S, and O;
L₄ is selected from 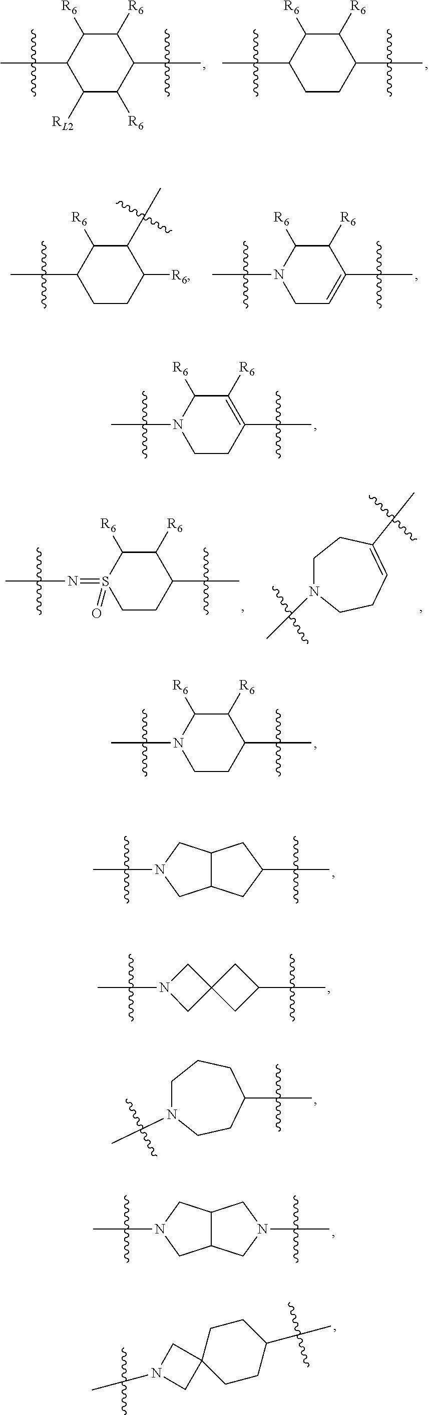 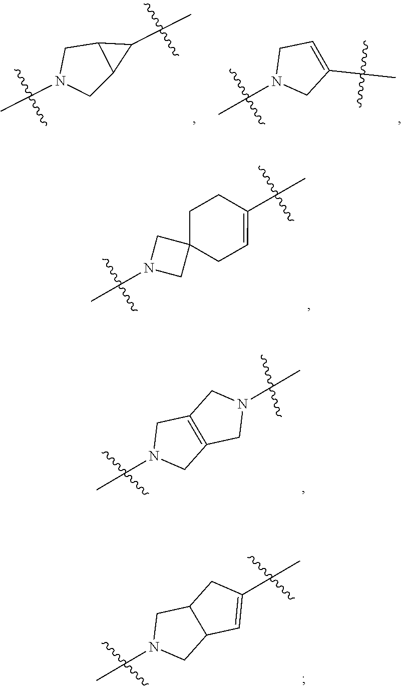 [SMILES: *C1C2CN(*)CC12 ; *C1=CCN(*)C1 ; *C1=CCC2(CC1)CN(*)C2 ; *N1CC2=C(C1)CN(*)C2 ; *C1=CC2CN(*)CC2C1]
A is —S(O)—, —C(O)—, —B(OH)—, —S—, or —S(═N)—CN;
Cy5 is selected from 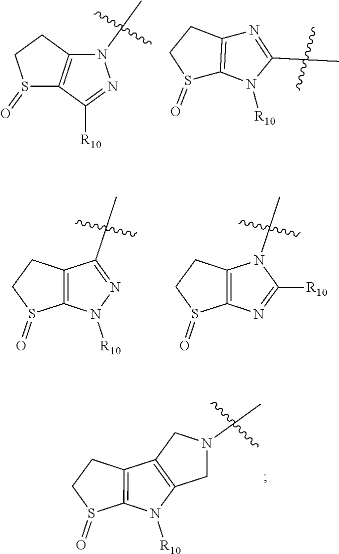
each R_{X1} and R are independently H, halogen, C₁₋₄ alkyl, C₁₋₄ alkoxy, —N(CH₃)₂, —NH(CH₃), C₂₋₄ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₆ cycloalkyl, or a 5- to 6-membered heterocycloalkyl containing 1-3 heteroatoms selected from N, S, O, and Si, wherein the cycloalkyl and heterocycloalkyl are optionally substituted with 1-3 groups selected from halogen, ═O, CN, C₁₋₄ alkyl, C₁₋₄ alkoxy, OH, and NH₂;
R₁ is C₁₋₄ alkyl or C₁₋₄ haloalkyl;
or R₁ and R₂, together with the atom to which they are attached, form a ring selected from: 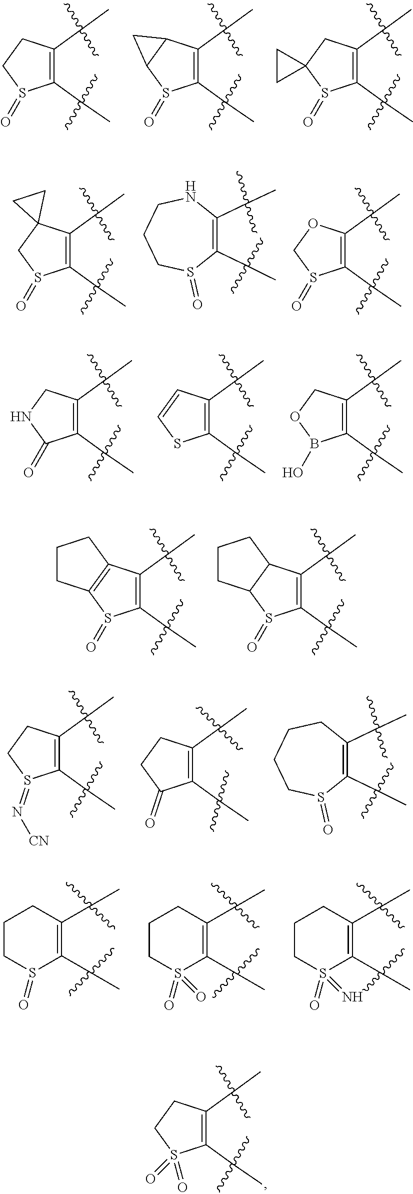
optionally substituted with 1-3 groups selected from halogen, ═O, CN, C₁₋₄ alkyl, C₁₋₄ alkoxy, OH, and NH₂;
R₃ is H, halogen, C₁₋₄ alkyl, CN, OH, or absent;
or R₃ and R₂, together with the atom to which they are attached, form a ring selected from: 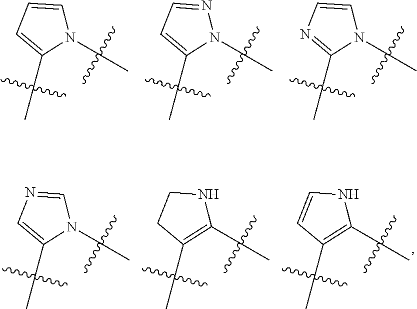 [SMILES: *c1cccn1* ; *c1ccnn1* ; *c1nccn1* ; *c1cncn1* ; *C1=C(*)NCC1 ; *c1cc[nH]c1*]
optionally substituted with 1-3 groups selected from halogen, ═O, CN, C₁₋₄ alkyl, C₁₋₄ alkoxy, OH, and NH₂;
or R₃ and R₄ or R₃ and R₅, together with the atom to which they are attached, form a 5- to 6-membered heterocycloalkyl or a 5- to 6-membered heteroaryl containing 1-3 heteroatoms selected from N, S, and O, or phenyl, wherein the heterocycloalkyl, phenyl, and heteroaryl are optionally substituted with 1-3 groups selected from halogen, ═O, CN, C₁₋₄ alkyl, C₁₋₄ alkoxy, OH, and NH₂;
or R₃ and R₆, together with the atom to which they are attached, form C₅₋₆ cycloalkyl, or a 5- to 6-membered heterocycloalkyl containing 1-3 heteroatoms selected from N, S, and O, optionally substituted with 1-3 groups selected from halogen, ═O, CN, C₁₋₄ alkyl, C₁₋₄ alkoxy, OH, and NH₂;
Rₓ₄ is H, halogen, C₁₋₄ alkyl, CN, OH, or absent; or Rₓ₄ and R₄ or Rₓ₄ and R₅, together with the atom to which they are attached, form imidazolyl, pyrazolyl, pyrrolyl, thienyl, or thiazolyl, optionally substituted with 1-3 groups selected from halogen, ═O, CN, C₁₋₄ alkyl, C₁₋₄ alkoxy, OH, and NH₂;
R₄ is H, C₁₋₄ alkyl, or C₁₋₄ haloalkyl;
R₅ is H, C₁₋₄ alkyl, C₁₋₄ haloalkyl, C₁₋₄ alkoxy, or —NHC₁₋₄ alkyl;
R₆ is H, halogen, ═O, CN, C₁₋₄ alkyl, or C₁₋₄ alkoxy;
R₇ is C₁₋₄ alkyl, or C₁₋₄ haloalkyl, or R₇ and R₉, together with the atom to which they are attached, form a ring selected from: 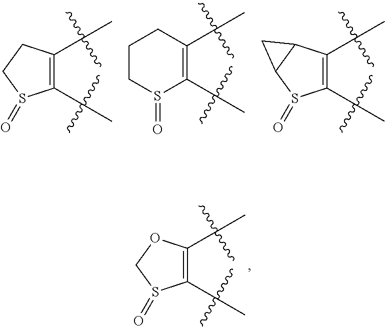
wherein the ring is optionally substituted with 1-3 groups selected from halogen, CN, C₁₋₄ alkyl, C₁₋₄ alkoxy, OH, and NH₂;
or R_{9′} and R₇, together with the atom to which they are attached, form a ring selected from: 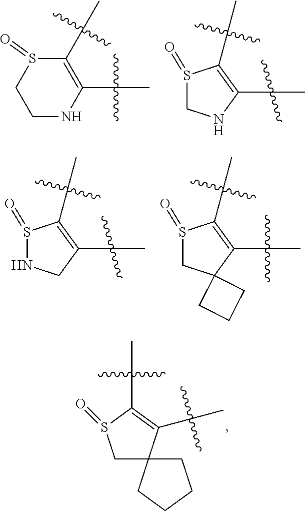 [SMILES: *C1=C(*)S(=O)CCN1 ; *C1=C(*)S(=O)CN1 ; *C1=C(*)S(=O)NC1 ; *C1=C(*)C2(CCC2)CS1=O ; *C1=C(*)C2(CCCC2)CS1=O]
optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′;}
or R_{9′} and Rₓ₅, together with the atom to which they are attached, form 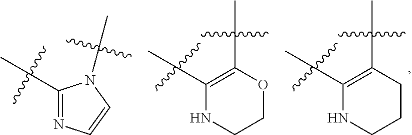 [SMILES: *c1nccn1* ; *C1=C(*)OCCN1 ; *C1=C(*)NCCC1]
optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, CN, or R_{8′};
provided that at least one group of R_{9′} and R₇ or R_{9′} and Rₓ₅ form a ring;
R₈ is R_{8′}, —OR_{8′}, or —(CH₂)ᵣR_{8′};
R_{8′} is C₃₋₆ cycloalkyl, a 4- to 8-membered heterocycloalkyl containing 1-3 heteroatoms selected from N, S, O, Si, and P, a 5- to 6-membered heteroaryl containing 1-3 heteroatoms selected from N, S, and O, —N═S(═O)(C₁₋₄ alkyl)₂, or —N(C₃₋₆ cycloalkyl)C(O)NH(C₁₋₄ alkyl), wherein the cycloalkyl, heterocycloalkyl, and heteroaryl are optionally substituted with 1-3 groups selected from C₁₋₄ alkyl, hydroxy C₁₋₄ alkyl, mercapto C₁₋₄ alkyl, halogen, halo C₁₋₄ alkyl, ═O, OH, NH₂, and CN;
R₉ is H or C₁₋₄ alkyl;
each R₁₀ is independently R_{8′}, —OR_{8′}, —NHR_{8′}, or —(CH₂)ᵣR_{8′};
R₁₁ is C₃₋₆ cycloalkyl optionally substituted with 1-3 groups selected from halogen, CN, ═O, OH, C₁₋₄ alkyl, hydroxy C₁₋₄ alkyl, and C₁₋₄ haloalkyl;
R₁₂ is selected from cyclobutyl, cyclopentyl, cyclohexyl, phenyl, cyclopropyl, adamantyl, tetrahydropyranyl, piperidinyl, oxolanyl, oxanyl, 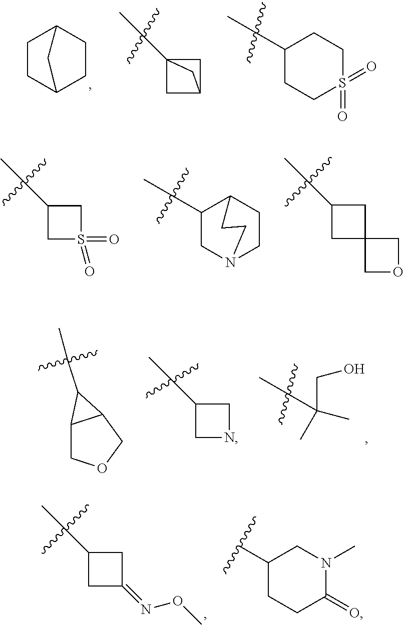
C₁₋₄ alkyl-CN, —C(NHC₁₋₂ alkyl)=N—CN, —C(C₁₋₂ alkyl)=N—CN, —C(NHC₁₋₂ alkyl)=CH—NO₂, —CH(CH₃)—CH₂—NH—C(O)O(CH₃), —C(CH₃)₂—CH₂—NH—C(O)O(CH₃), —CH(CH₃)—CH₂—O—C(O)NH₂, —C(CH₃)₂—CH₂—O—C(O)NH₂, —C(O)—Ra, —CH(CH₃)—CH₂—O—C(O)NHCH₃, or —C(CH₃)₂—CH₂—O—C(O)NHCH₃; wherein the ring is optionally substituted with 1-3 groups selected from halogen, CN, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NH—C(O)NH₂, —(CH₂)ₜ—NH—C(═NH)NH₂, —(CH₂)ₜ—NH—C(O)—O—(C₁₋₂ alkyl), —(CH₂)ₜ—NH—C(C₁₋₂ alkyl)=N—CN, —(CH₂)ₜ—NH—C(NHC₁₋₂ alkyl)=N—CN, —(CH₂)ₜ—NH—C(NHC₁₋₂ alkyl)=CH—NO₂, —(CH₂)ₜ—C(NHC₁₋₂ alkyl)=N—CN, —(CH₂)ₜ—C(C₁₋₂ alkyl)=N—CN, —(CH₂)ₜ—C(C₁₋₂ alkyl)=N—O—(C₁₋₂ alkyl), ═N—O—C₁₋₂ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃;
R₁₃ is selected from 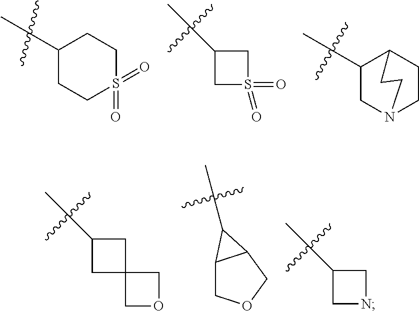
wherein the ring is optionally substituted with 1-3 groups selected from halogen, CN, OH, C₁₋₄ alkyl, hydroxy C₁₋₄ alkyl, and C₁₋₄ haloalkyl;
n is 0, 1, 2, or 3;
m is 0, 1, or 2;
r is 1 or 2; and
t is selected from 0, 1, or 2.

4. The compound or the stereoisomer or pharmaceutically acceptable salt thereof according to claim 1, wherein
Cy1 is selected from 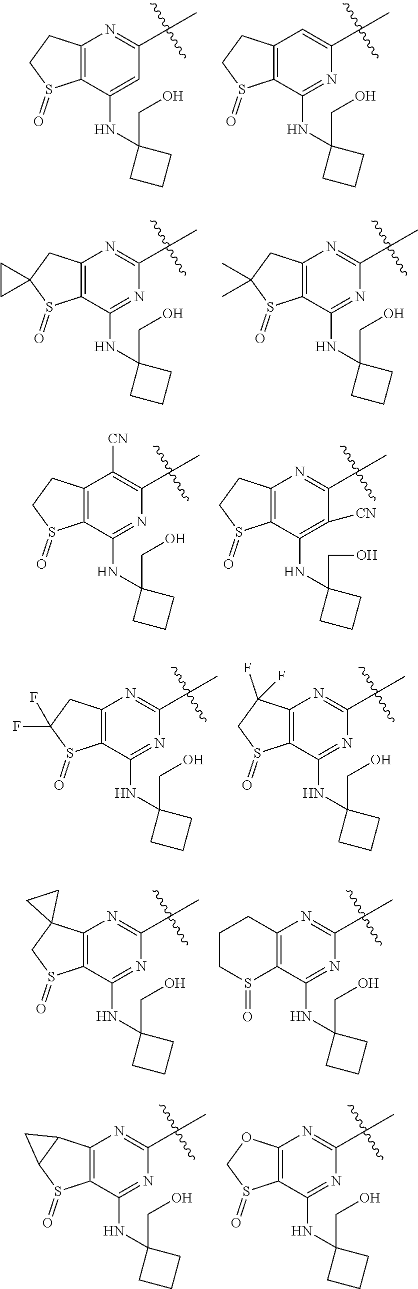 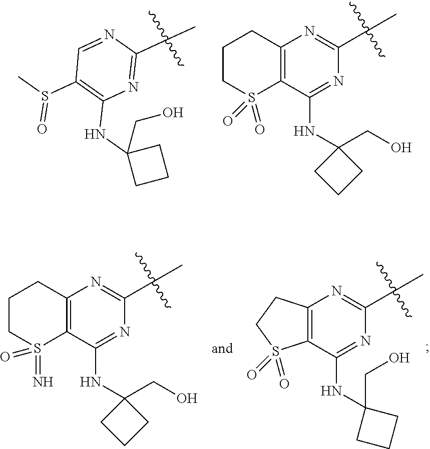 [SMILES: *c1ncc(S(C)=O)c(NC2(CO)CCC2)n1 ; *c1nc2c(c(NC3(CO)CCC3)n1)S(=O)(=O)CCC2 ; *c1nc2c(c(NC3(CO)CCC3)n1)S(=N)(=O)CCC2 ; *c1nc2c(c(NC3(CO)CCC3)n1)S(=O)(=O)CC2]
Cy2 is selected from 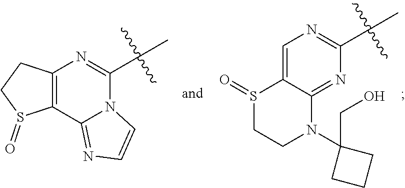 [SMILES: *c1nc2c(c3nccn13)S(=O)CC2 ; *c1ncc2c(n1)N(C1(CO)CCC1)CCS2=O]
Cy4 is selected from 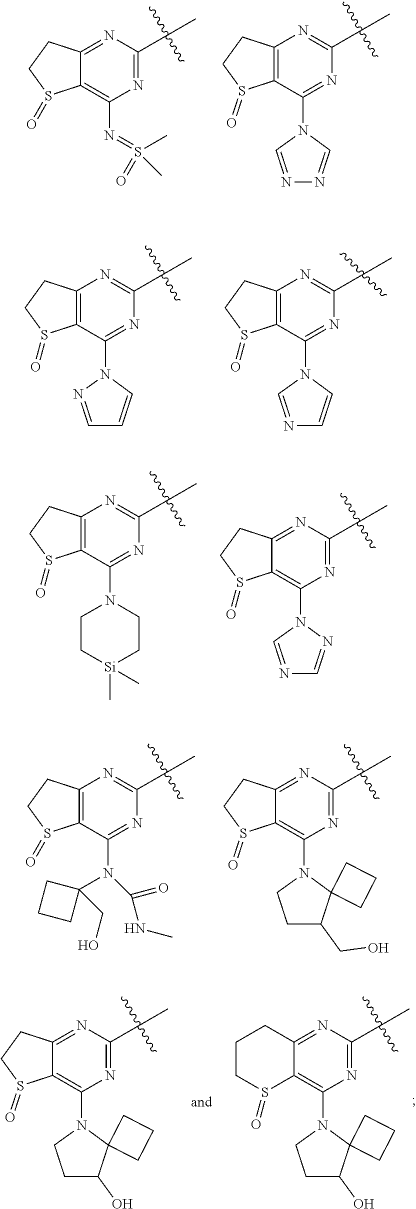
Cy5 is 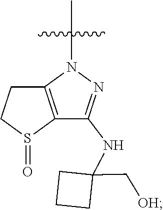 [SMILES: *n1nc(NC2(CO)CCC2)c2c1CCS2=O]
Cy7 is selected from 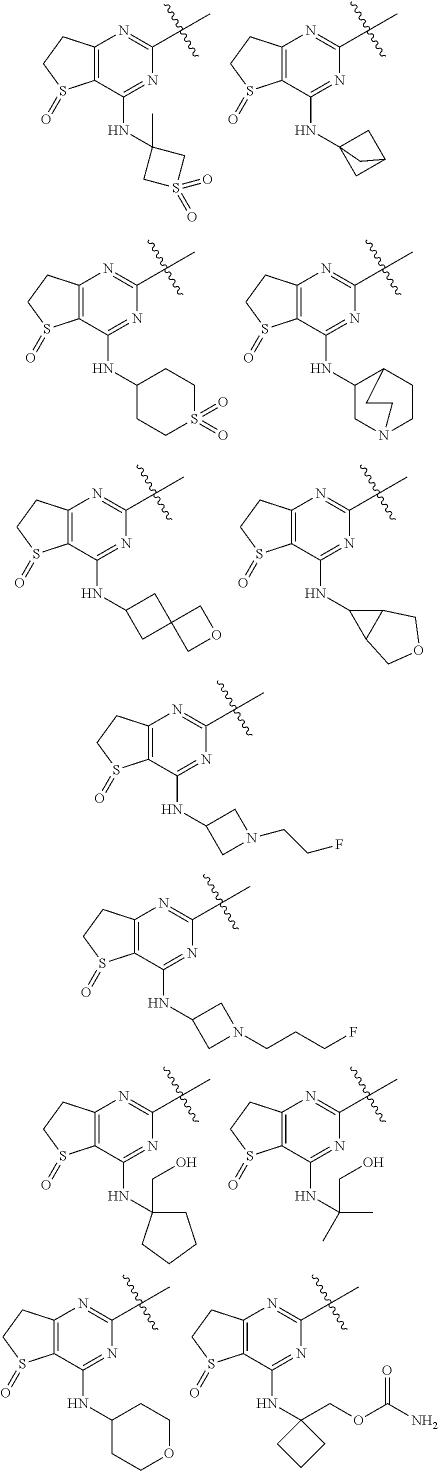 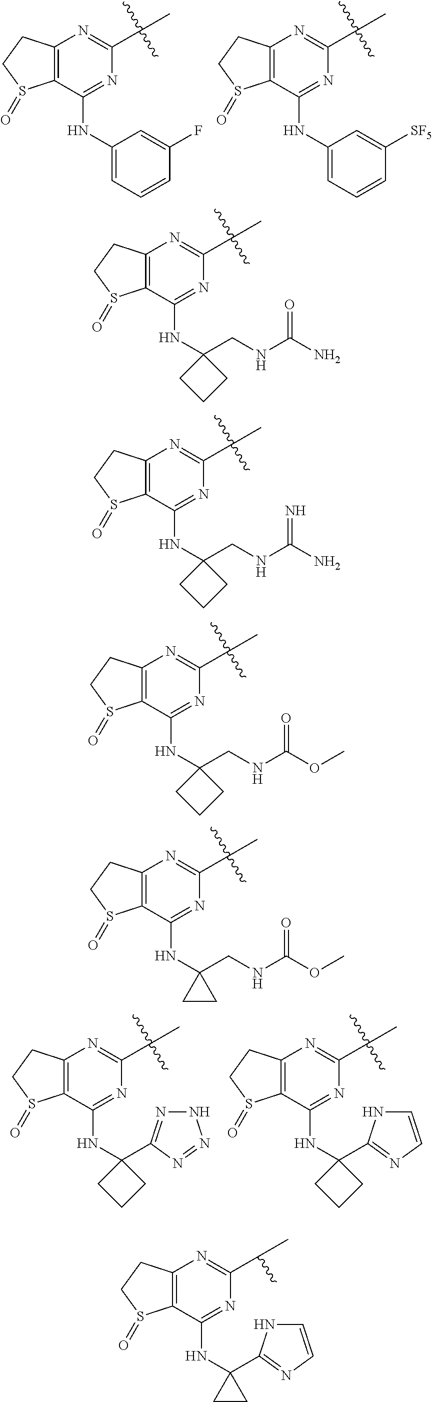 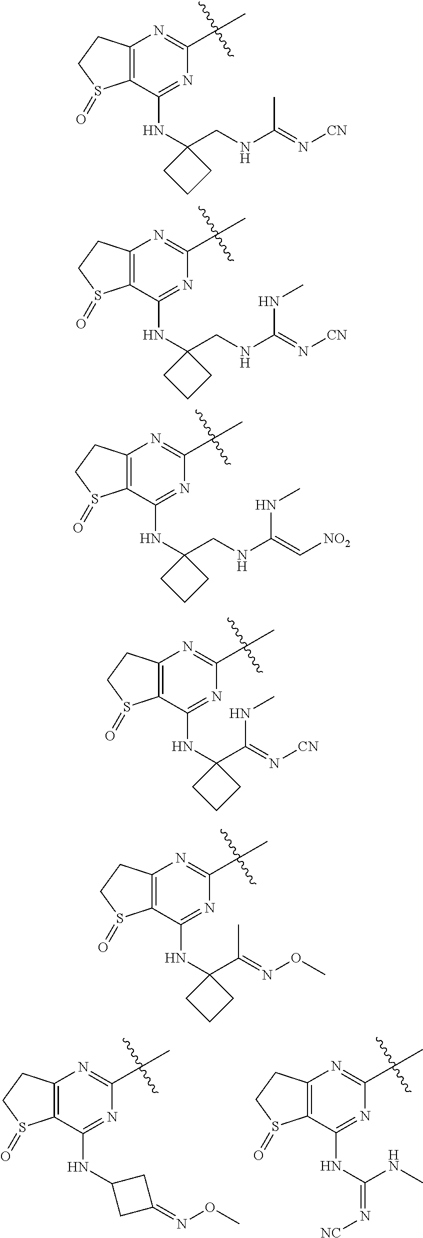 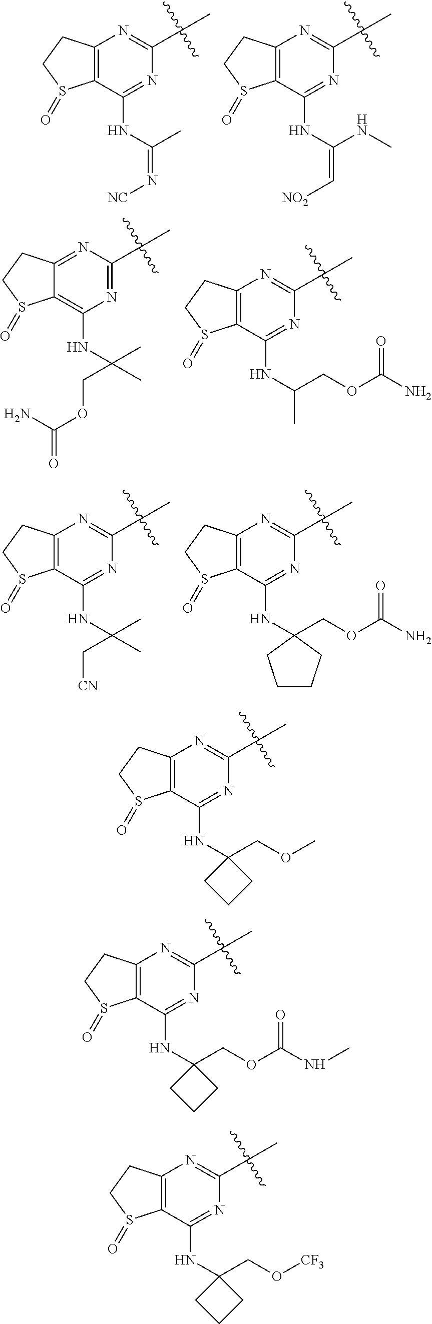 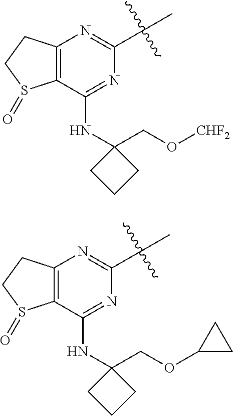 [SMILES: *c1nc2c(c(NC3(COC(F)F)CCC3)n1)S(=O)CC2 ; *c1nc2c(c(NC3(COC4CC4)CCC3)n1)S(=O)CC2] 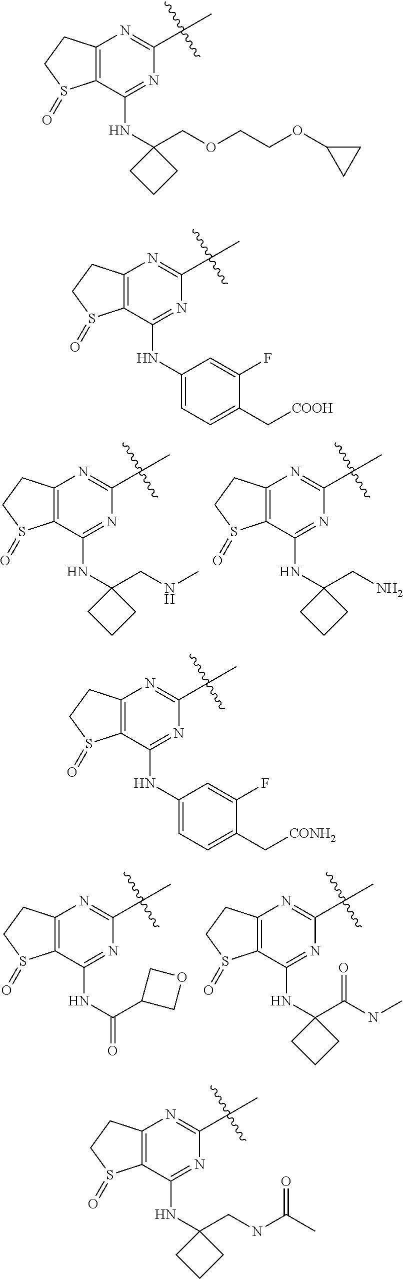 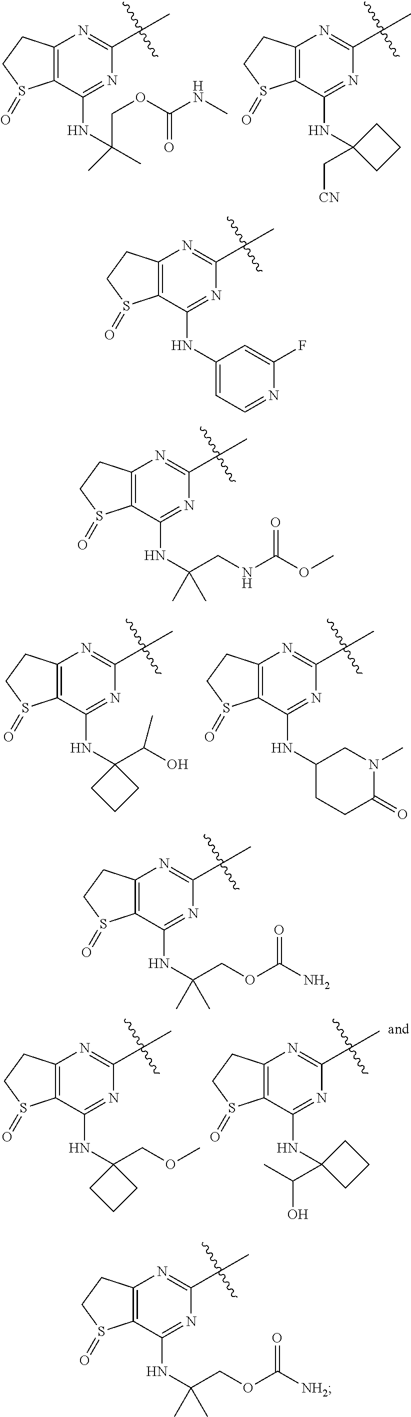
and
Cy9 is

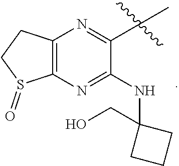 [SMILES: *c1nc2c(nc1NC1(CO)CCC1)S(=O)CC2]

5. The compound or the stereoisomer or pharmaceutically acceptable salt thereof according to claim 1, wherein
-(L₁)n-(L₂)m- is selected from: 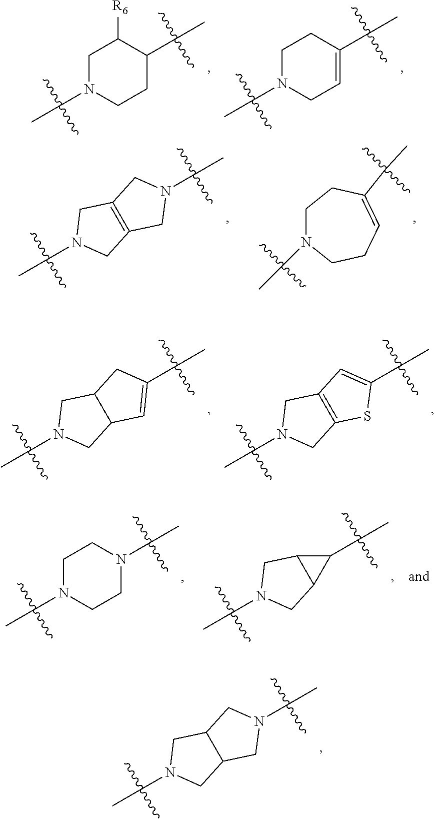
with R₆ being H;
-L₃- is selected from: 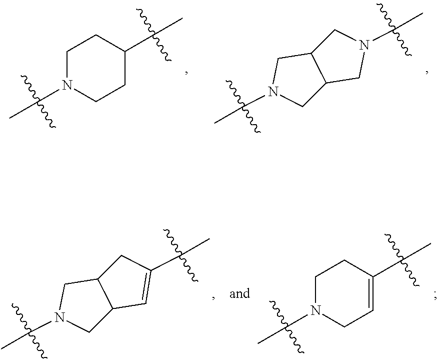 [SMILES: *C1CCN(*)CC1 ; *N1CC2CN(*)CC2C1 ; *C1=CC2CN(*)CC2C1 ; *C1=CCN(*)CC1]
-L₄- is selected from: 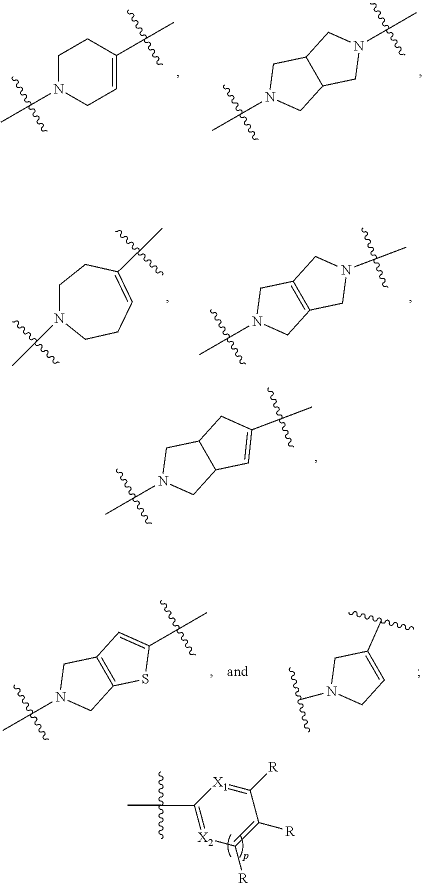
is selected from one of the following structures optionally substituted with 1-3 R_{R}: 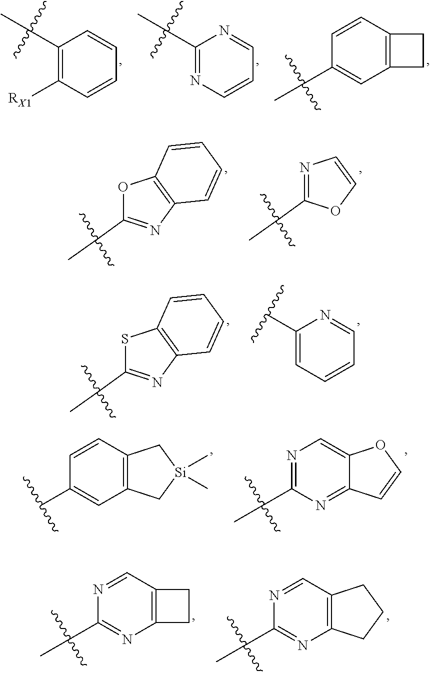
and each R_{R} is independently selected from F, Cl, Br, methyl, isopropyl, ethoxy, methoxy, ethynyl, propynyl, cyclopropyl, cyclobutyl, dimethylamino, and 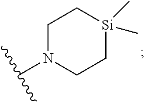 [SMILES: *N1CC[Si](C)(C)CC1]
and R_{X1} is H;
or R_{X1} and R₆, together with the atom to which they are attached, form morpholinyl, furyl, pyrrolyl, or thienyl.

6. The compound or the stereoisomer or pharmaceutically acceptable salt thereof according to claim 1, 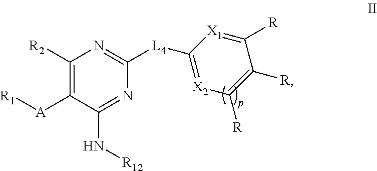
wherein L₄ is 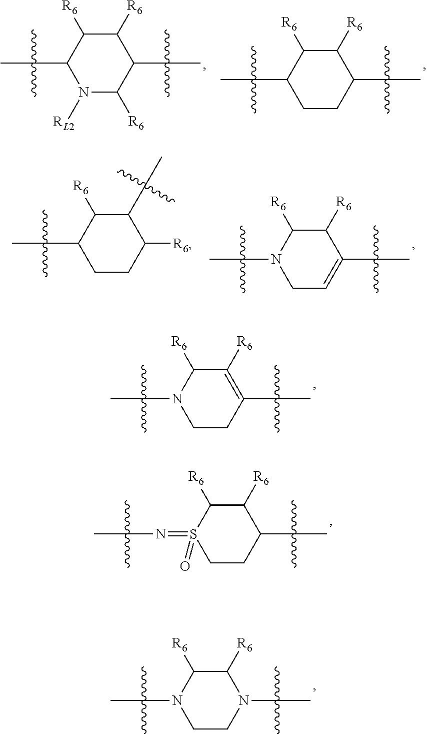
a 5- to 6-membered heteroaryl containing 1-3 heteroatoms selected from N, S, and O, a 5-membered monocyclic heterocycloalkyl containing 1-3 heteroatoms selected from N, S, and O, a 7- to 8-membered monocyclic heterocycloalkyl containing 1-3 heteroatoms selected from N, S, and O, a 5- to 6-membered heterocycloalkyl fused 5- to 6-membered heterocycloalkyl containing 1-3 heteroatoms selected from N, S, and O, a 5- to 6-membered heterocycloalkyl fused 5- to 6-membered cycloalkyl containing 1-3 heteroatoms selected from N, S, and O, or a 7- to 12-membered spiro heterocycloalkyl containing 1-3 heteroatoms selected from N, S, and O, wherein the monocyclic heterocycloalkyl, 5- to 6-membered heterocycloalkyl fused 5- to 6-membered heterocycloalkyl, 5- to 6-membered heterocycloalkyl fused 5- to 6-membered cycloalkyl, and spiro heterocycloalkyl are optionally substituted with 1-3 groups selected from halogen, ═O, CN, C₁₋₄ alkyl, C₁₋₄ alkoxy, OH, and NH₂;
A is —S(O)—, —C(O)—, —B(OH)—, —S—, —S(═N)—CN, or —C(═N)—CN;
X₁ and X₂ are independently CR_{X1}, S, or N;
R_{L2} is H, C₁₋₄ alkyl, or C₃₋₆ cycloalkyl;
R₆ is H, halogen, ═O, CN, C₁₋₄ alkyl, or C₁₋₄ alkoxy;
each R_{X1} and R are independently H, halogen, C₁₋₄ alkyl, C₁₋₄ alkoxy, —N(CH₃)₂, —NH(CH₃), C₂₋₆ alkynyl, NHSO₂NH₂, NHCONH₂, NHCOC₁₋₄ alkyl, CONHC₁₋₄ alkyl, NHSO₂C₁₋₄ alkyl, SO₂NHC₁₋₄ alkyl, SO₂C₁₋₄ alkyl, SCF₃, SF₅, a 3- to 5-membered cycloalkyl, C₁₋₄ haloalkyl, or a 5- to 10-membered heterocycloalkyl containing 1-3 heteroatoms selected from N, S, O, and Si, or two R on adjacent ring atoms, R and R_{X1}, or R_{X1} and R₆, together with the atom to which they are attached, form C₃₋₈ cycloalkyl, a 5- to 10-membered heterocycloalkyl containing 1-3 heteroatoms selected from N, S, O, and Si, phenyl, or a 5- to 6-membered heteroaryl containing 1-3 heteroatoms selected from N, S, and O, wherein the cycloalkyl, heterocycloalkyl, phenyl, and heteroaryl are optionally substituted with 1-3 groups selected from halogen, ═O, CN, C₁₋₄ alkyl, C₁₋₄ alkoxy, OH, and NH₂;
R₁ and R₂, together with the atom to which they are attached, form a 5- to 10-membered heterocycloalkyl or a 5- to 6-membered heteroaryl containing 1-3 heteroatoms selected from N, S, B, and O, wherein the heterocycloalkyl; and heteroaryl are optionally substituted with 1-3 groups selected from halogen, ═O, CN, C₁₋₄ alkyl, C₁₋₄ alkoxy, OH, and NH₂;
R₁₂ is selected from a 4- to 10-membered heterocycloalkylene, a 3- to 10-membered cycloalkylene, a 6- to 10-membered arylene, a 5- to 10-membered heteroarylene, hydroxy-C₁₋₆ alkyl, C₁₋₄ alkyl-CN, —CRaRb=N—O—C₁₋₄ alkyl, —C(NRaRb)=N—CN, —C(C₁₋₄ alkyl)=N—CN, —C(NRaRb)=CRa—NO₂, —C(CH₃)₂—CH₂—O—C(O)NH₂, —C(CH₃)₂—CH₂—NH—C(O)O(C₁₋₄ alkyl), —C(CH₃)₂—CH₂—O—C(O)NHCH₃, —CH(CH₃)—CH₂—O—C(O)NH₂, —CH(CH₃)—CH₂—NH—C(O)O(C₁₋₄ alkyl), —CH(CH₃)—CH₂—O—C(O)NHCH₃, —C(O)—Ra, or —C(O)-(4- to 6-membered heterocycloalkyl), wherein the heterocycloalkylene, cycloalkylene, arylene, and heteroarylene are optionally further substituted with 1-3 groups selected from halogen, CN, ═O, OH, —SF₅, C₁₋₄ alkyl, C₁₋₄ alkyl-CN, hydroxy C₁₋₄ alkyl, C₁₋₄ haloalkyl, —(CH₂)ₜ—O—C(O)NH₂, —(CH₂)ₜ—NRa—C(O)NH₂, —(CH₂)ₜ—NRa—C(═NH)NH₂, —(CH₂)ₜ—NRa—C(O)—O—(C₁₋₄ alkyl), —(CH₂)ₜ—NRa—C(C₁₋₄ alkyl)=N—CN, —(CH₂)ₜ—NRa—C(NRaRb)=N—CN, —(CH₂)ₜ—NRa—C(NRaRb)=CRa—NO₂, —(CH₂)ₜ—C(NRaRb)=N—CN, —(CH₂)ₜ—C(C₁₋₄ alkyl)=N—CN, —(CH₂)ₜ—C(C₁₋₄ alkyl)=N—O—(C₁₋₄ alkyl), ═N—O—C₁₋₄ alkyl, a 5- to 6-membered heteroaryl, —(CH₂)ₜ—O—C₁₋₄ alkyl, —(CH₂)ₜ—O—C(O)NHCH₃, —(CH₂)ₜ—O—C₁₋₂ haloalkyl, —(CH₂)ₜ—O—C₃₋₆ cycloalkyl, —(CH₂)ₜ—O—C₁₋₄ alkyl-O—C₃₋₆ cycloalkyl, —(CH₂)ₜ—COOH, —(CH₂)ₜ—NRaRb, —(CH₂)ₜ—C(O)NRaRb, and —(CH₂)ₜ—NRa—C(O)CH₃;
Ra and Rb are each independently selected from H, halogen, C₁₋₄ alkyl, C₁₋₄ haloalkyl, C₁₋₄ alkoxy, halo C₁₋₄ alkoxy, or a 4- to 6-membered heterocycloalkyl containing 1-2 heteroatoms selected from N and O; and
p is selected from 0 or 1.

7. The compound or the stereoisomer or pharmaceutically acceptable salt thereof according to claim 6,
wherein L₄ is 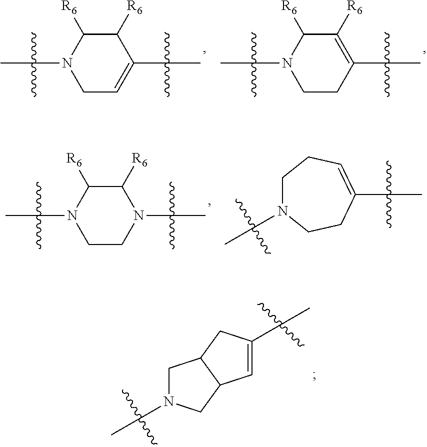
and
R₁₂ is selected from —C(CH₃)₂—CH₂—O—C(O)NH₂, —C(CH₃)₂—CH₂—NH—C(O)O(C₁₋₄ alkyl), —C(CH₃)₂—CH₂—O—C(O)NHCH₃, —CH(CH₃)—CH₂—O—C(O)NH₂, —CH(CH₃)—CH₂—NH—C(O)O(C₁₋₄ alkyl), and —CH(CH₃)—CH₂—O—C(O)NHCH₃.

8. The compound or the stereoisomer or pharmaceutically acceptable salt thereof according to claim 1, wherein the compound is selected from one of the structures in Table 1.

9. The compound or the stereoisomer or pharmaceutically acceptable salt thereof according to claim 1, wherein the compound is selected from one of the structures in Table 2.

10. A pharmaceutical composition or pharmaceutical preparation comprising the compound or the stereoisomer or pharmaceutically acceptable salt thereof according to claim 1, and a pharmaceutically acceptable carrier and/or auxiliary material.

11. The pharmaceutical composition or pharmaceutical preparation according to claim 10, wherein the pharmaceutical composition or pharmaceutical preparation comprises 1-1500 mg of the compound or the stereoisomer or pharmaceutically acceptable salt thereof, and a pharmaceutically acceptable carrier and/or auxiliary material.

12. (canceled)

13. A method for treating a disease in a mammal or human, comprising administering to a subject a therapeutically effective amount of the compound or the stereoisomer or pharmaceutically acceptable salt thereof according to claim 1.

14. The method according to claim 13, wherein the disease is a PDE4B-mediated disease.

15. The method according to claim 13, wherein the therapeutically effective amount is 1-1500 mg.

16. The method according to claim 13, wherein the disease is a cancer, COPD, idiopathic pulmonary fibrosis, or an interstitial lung disease.
